(19) United States
(12) Patent Application Publication
(10) Pub. No.: US 2023/0226096 A1
(43) Pub. Date: Jul. 20, 2023

(54) CIRCULAR RNA COMPOSITIONS AND METHODS

(71) Applicant: Orna Therapeutics, Inc., Cambridge, MA (US)

(72) Inventors: Brian Goodman, Jamaica Plain, MA (US); Robert Alexander Wesselhoeft, Cambridge, MA (US); Allen T. Horhota, Cambridge, MA (US); Junghoon Yang, Cambridge, MA (US); Kristen Ott, Cambridge, MA (US); Thomas Barnes, Cambridge, MA (US)

(21) Appl. No.: 17/998,219

(22) PCT Filed: May 10, 2021
(86) PCT No.: PCT/US2021/031629

§ 371 Date: Nov. 8, 2022

Related U.S. Application Data
(60) Provisional application No. 63/087,582, filed on Oct. 5, 2020.
(60) Provisional application No. 63/022,248, filed on May 8, 2020.

(51) Int. Cl. A61K 31/713; A61K 9/127; C12N 15/88

(52) U.S. Cl. CPC A61K 31/713; A61K 9/1271; A61K 9/1272; C12N 15/88

(57) ABSTRACT

Disclosed herein are circular RNAs and transfer vehicles, along with related compositions and methods of treatment. The circular RNAs can comprise group I intron fragments, spacers, an IRES, duplex forming regions, and/or an expression sequence, thereby having the features of improved expression, functional stability, low immunogenicity, ease of manufacturing, and/or extended half-life compared to linear RNA. Pharmaceutical compositions comprising such circular RNAs and transfer vehicles are particularly suitable for efficient protein expression in immune cells in vivo. Also disclosed are precursor RNAs and materials useful in producing the precursor or circular RNAs, which have improved circularization efficiency and/or are compatible with effective circular RNA purification methods.

### CROSS REFERENCE TO RELATED APPLICATIONS

This application claims the benefit of, and priority to, U.S. Provisional Application No. 63/022,248, filed on May 8, 2020; U.S. Provisional Application No. 63/087,582, filed on Oct. 5, 2020; and International Patent Application No. PCT/US2020/063494, filed on Dec. 4, 2020, the contents of each of which are hereby incorporated by reference in their entirety for all purposes.

### BACKGROUND

Conventional gene therapy involves the use of DNA for insertion of desired genetic information into host cells. The DNA introduced into the cell is usually integrated to a certain extent into the genome of one or more transfected cells, allowing for long-lasting action of the introduced genetic material in the host. While there may be substantial benefits to such sustained action, integration of exogenous DNA into a host genome may also have many deleterious effects. For example, it is possible that the introduced DNA will be inserted into an intact gene, resulting in a mutation which impedes or even totally eliminates the function of the endogenous gene. Thus, gene therapy with DNA may result in the impairment of a vital genetic function in the treated host, such as, e.g., elimination or deleteriously reduced production of an essential enzyme or interruption of a gene critical for the regulation of cell growth, resulting in unregulated or cancerous cell proliferation. In addition, with conventional DNA based gene therapy it is necessary for effective expression of the desired gene product to include a strong promoter sequence, which again may lead to undesirable changes in the regulation of normal gene expression in the cell. It is also possible that the DNA based genetic material will result in the induction of undesired anti-DNA antibodies, which in turn, may trigger a possibly fatal immune response. Gene therapy approaches using viral vectors can also result in an adverse immune response. In some circumstances, the viral vector may even integrate into the host genome. In addition, production of clinical grade viral vectors is also expensive and time consuming. Targeting delivery of the introduced genetic material using viral vectors can also be difficult to control. Thus, while DNA based gene therapy has been evaluated for delivery of secreted proteins using viral vectors (U.S. Pat. No. 6,066,626; US2004/0110709), these approaches may be limited for these various reasons.

In contrast to DNA, the use of RNA as a gene therapy agent is substantially safer because RNA does not involve the risk of being stably integrated into the genome of the transfected cell, thus eliminating the concern that the introduced genetic material will disrupt the normal functioning of an essential gene, or cause a mutation that results in deleterious or oncogenic effects, and extraneous promoter sequences are not required for effective translation of the encoded protein, again avoiding possible deleterious side effects. In addition, it is not necessary for mRNA to enter the nucleus to perform its function, while DNA must overcome this major barrier.

Circular RNA is useful in the design and production of stable forms of RNA. The circularization of an RNA molecule provides an advantage to the study of RNA structure and function, especially in the case of molecules that are prone to folding in an inactive conformation (Wang and Ruffner, 1998). Circular RNA can also be particularly interesting and useful for in vivo applications, especially in the research area of RNA-based control of gene expression and therapeutics, including protein replacement therapy and vaccination.

Prior to this invention, there were three main techniques for making circularized RNA in vitro: the splint-mediated method, the permuted intron-exon method, and the RNA ligase-mediated method. However, the existing methodologies are limited by the size of RNA that can be circularized, thus limiting their therapeutic application.

### SUMMARY

The present application provides circular RNAs and transfer vehicles, along with related compositions and methods of treatment. The transfer vehicles can comprise, e.g., ionizable lipid, PEG-modified lipid, and/or structural lipid, thereby forming lipid nanoparticles encapsulating the circular RNAs. The circular RNAs can comprise group I intron fragments, spacers, an IRES, duplex forming regions, and/or an expression sequence, thereby having the features of improved expression, functional stability, low immunogenicity, ease of manufacturing, and/or extended half-life compared to linear RNA. Pharmaceutical compositions comprising such circular RNAs and transfer vehicles are particularly suitable for efficient protein expression in immune cells in vivo. The present application also provides precursor RNAs and materials useful in producing the precursor or circular RNAs, which have improved circularization efficiency and/or are compatible with effective circular RNA purification methods.

Accordingly, one aspect of the present application provides a pharmaceutical composition comprising a circular RNA polynucleotide and a transfer vehicle comprising an ionizable lipid represented by Formula (1):

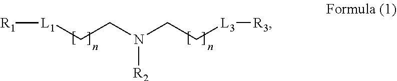

wherein:

each n is independently an integer from 2-15;

L₁ and L₃ are each independently —OC(O)—* or —C(O)O—*, wherein “*” indicates the attachment point to R₁ or R₃;

R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkyl sulfoxidealkyl, alkylsulfonyl, and alkylsulfonealkyl; and

R₂ is selected from a group consisting of:

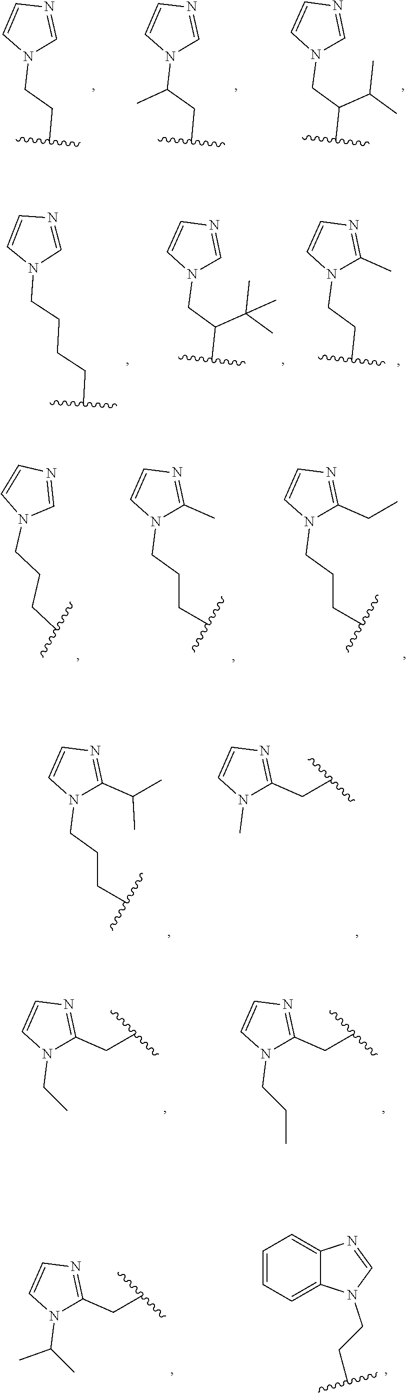

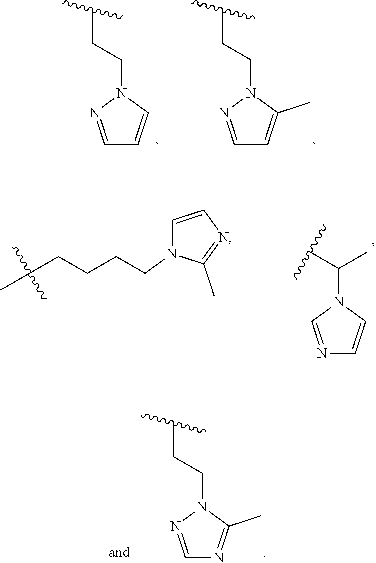

In some embodiments, the circular RNA polynucleotide is encapsulated in the transfer vehicle. In some embodiments, the circular RNA polynucleotide is encapsulated in the transfer vehicle with an encapsulation efficiency of at least 80%. In some embodiments, the transfer vehicle has a diameter of about 56 nm or larger. In some embodiments, the transfer vehicle has a diameter of about 56 nm to about 157 nm.

In some embodiments, R₁ and R₃ are each independently selected from a group consisting of:

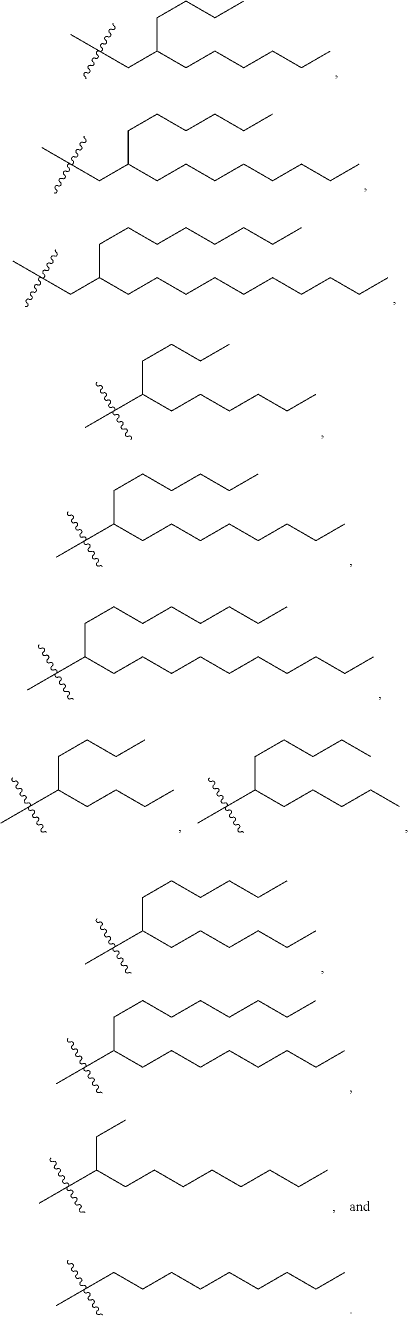

In some embodiments, R₁ and R₃ are the same. In some embodiments, R₁ and R₃ are different.

In some embodiments, the ionizable lipid of Formula (1) is represented by Formula (1-1) or Formula (1-2):

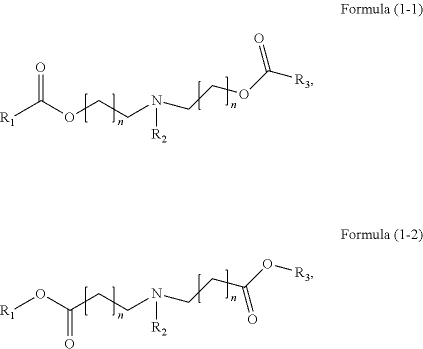

In some embodiments, the ionizable lipid is selected from the group consisting of:

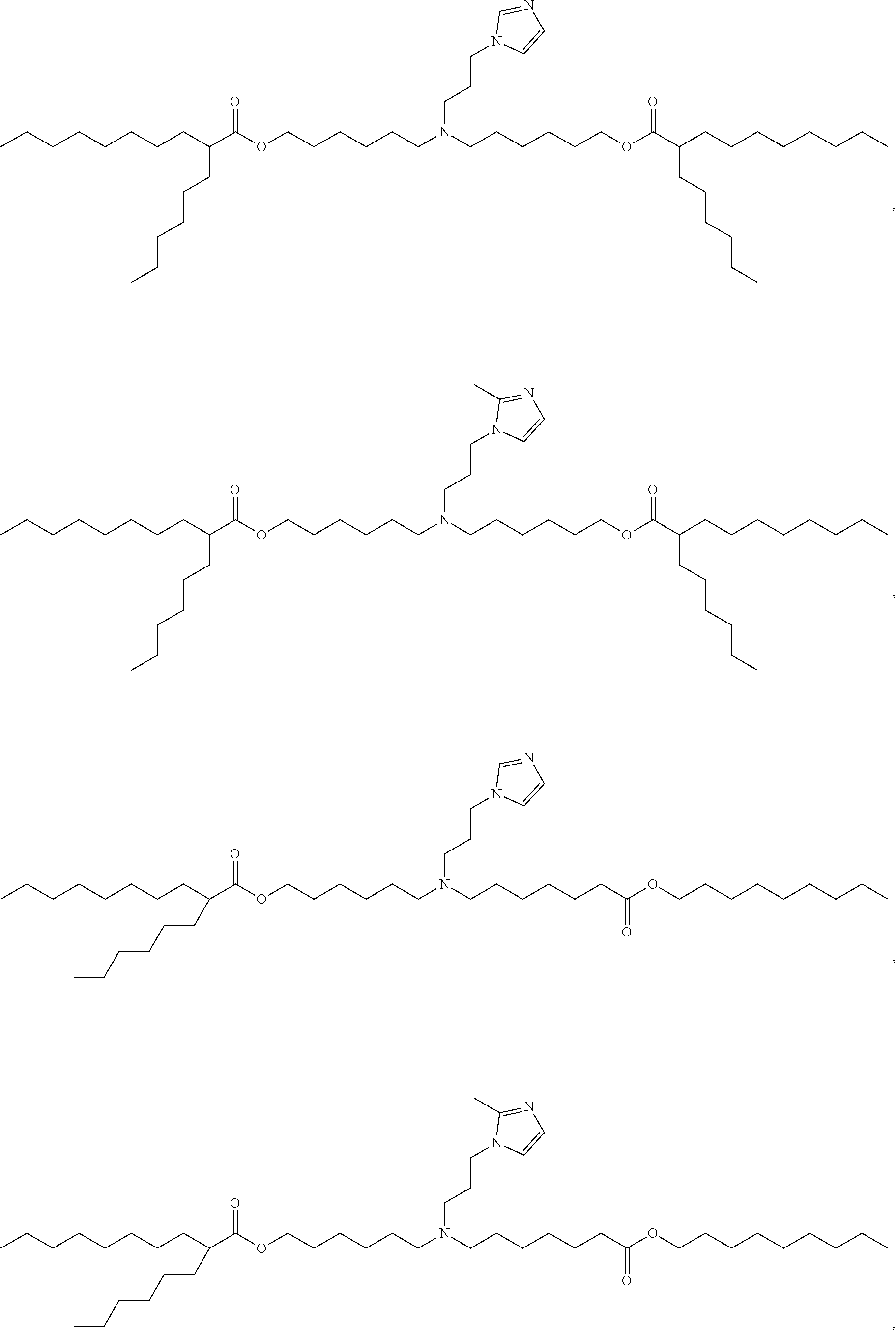

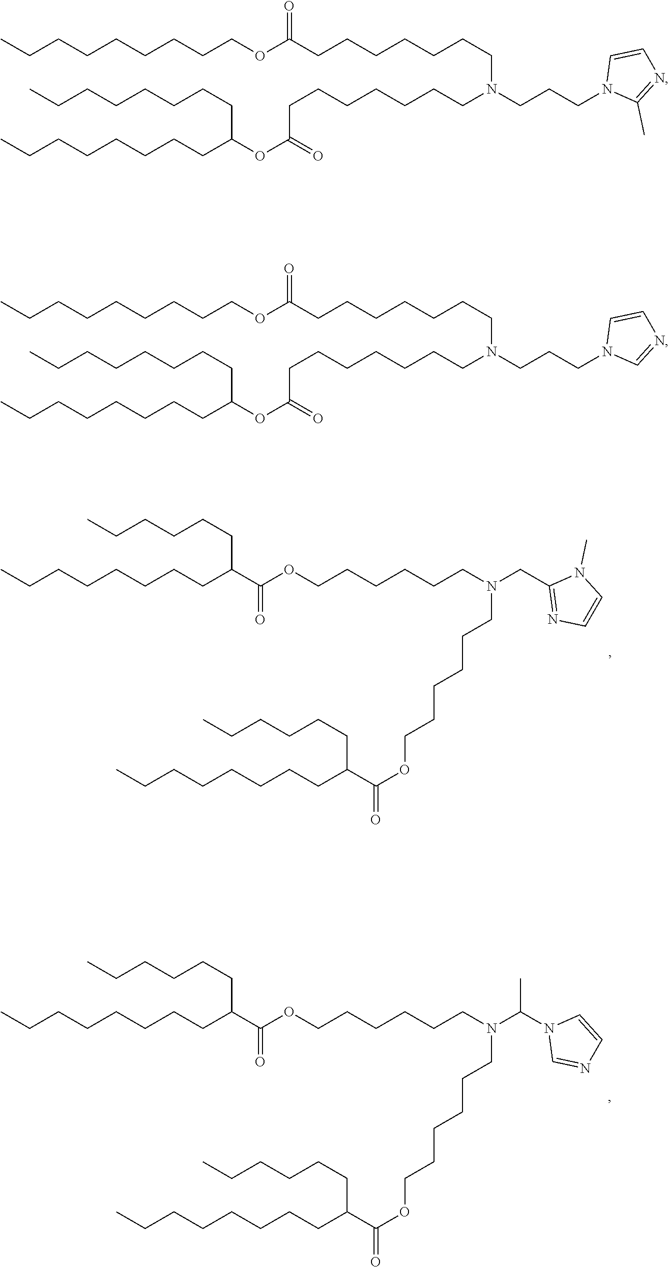 [SMILES: CCCCCCCCCOC(=O)CCCCCCCN(CCCCCCCC(=O)OC(CCCCCCCC)CCCCCCCC)CCCn1ccnc1C ; CCCCCCCCCOC(=O)CCCCCCCN(CCCCCCCC(=O)OC(CCCCCCCC)CCCCCCCC)CCCn1ccnc1 ; CCCCCCCCC(CCCCCC)C(=O)OCCCCCCN(CCCCCCOC(=O)C(CCCCCC)CCCCCCCC)Cc1nccn1C ; CCCCCCCCC(CCCCCC)C(=O)OCCCCCCN(CCCCCCOC(=O)C(CCCCCC)CCCCCCCC)C(C)n1ccnc1]

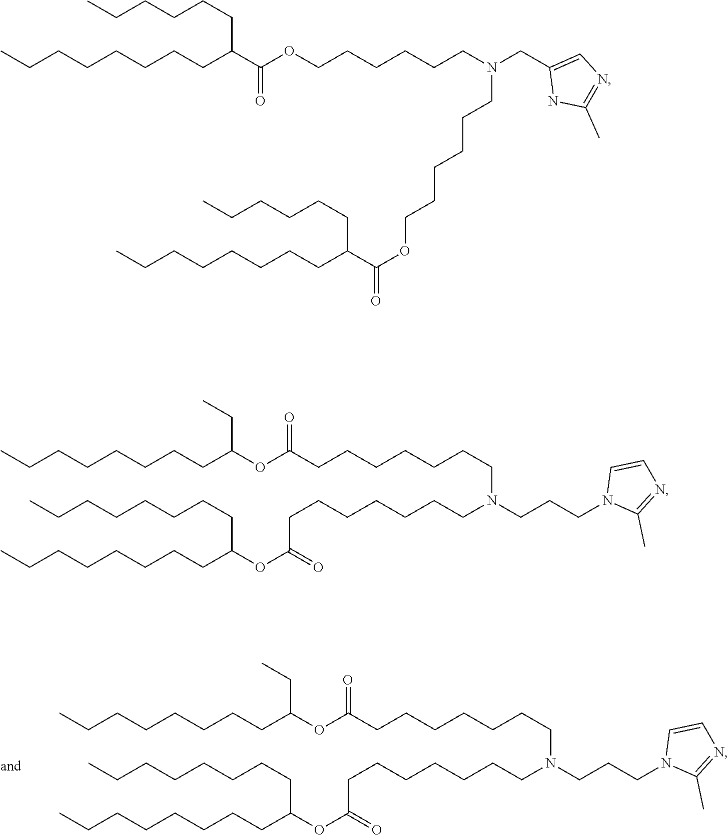 [SMILES: CCCCCCCCC(CCCCCC)C(=O)OCCCCCCN(CCCCCCOC(=O)C(CCCCCC)CCCCCCCC)Cc1cnc(C)[nH]1 ; CCCCCCCCC(CC)OC(=O)CCCCCCCN(CCCCCCCC(=O)OC(CCCCCCCC)CCCCCCCC)CCCn1ccnc1C ; CCCCCCCCC(CC)OC(=O)CCCCCCCN(CCCCCCCC(=O)OC(CCCCCCCC)CCCCCCCC)CCCn1ccnc1C]

In another aspect, the present application provides a pharmaceutical composition comprising: a circular RNA polynucleotide and a transfer vehicle comprising an ionizable lipid represented by Formula (2):

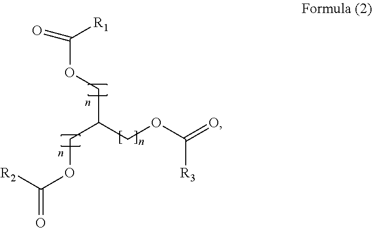

wherein:

each n is independently an integer from 1-15;

R₁ and R₂ are each independently selected from a group consisting of:

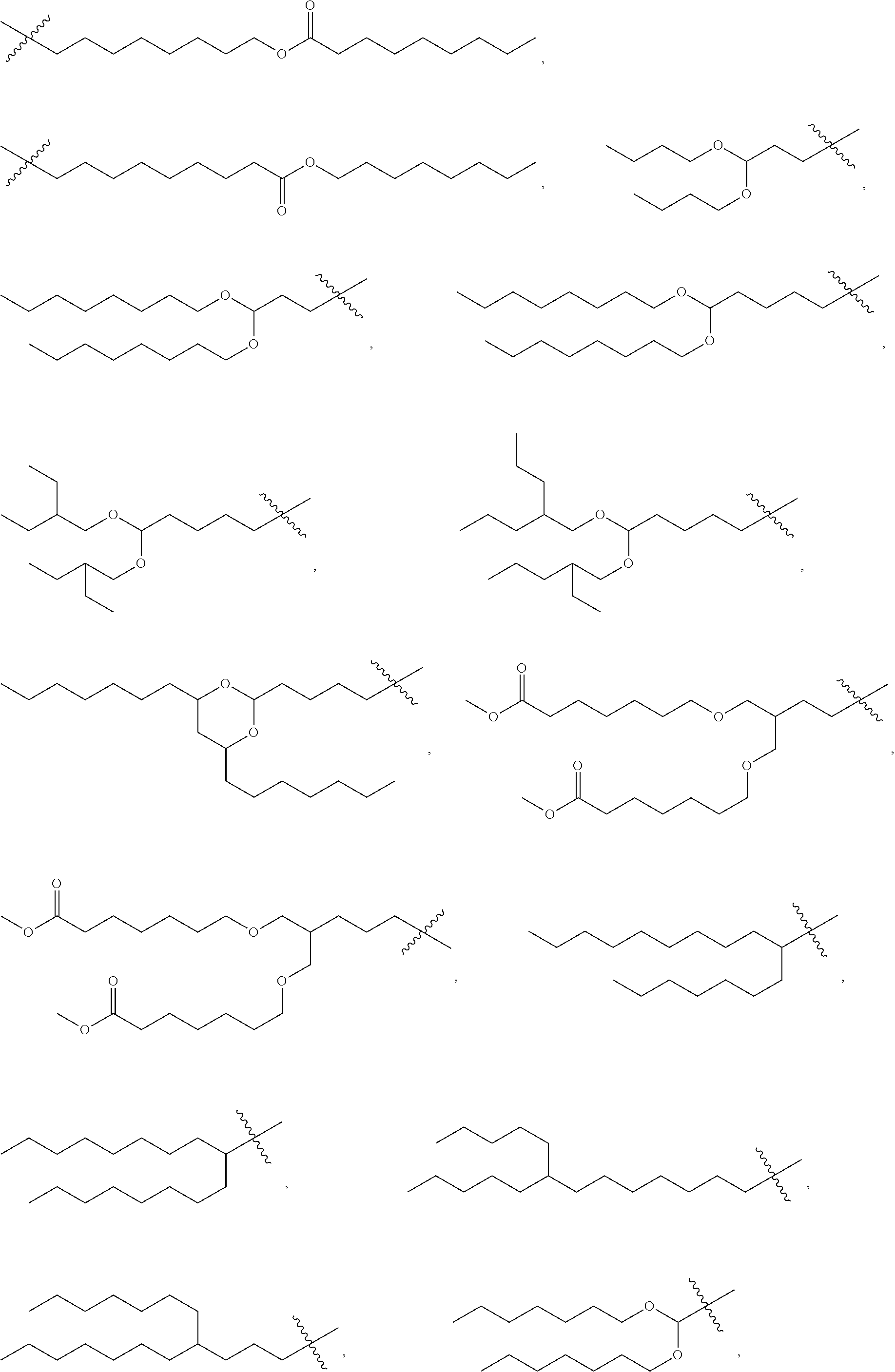

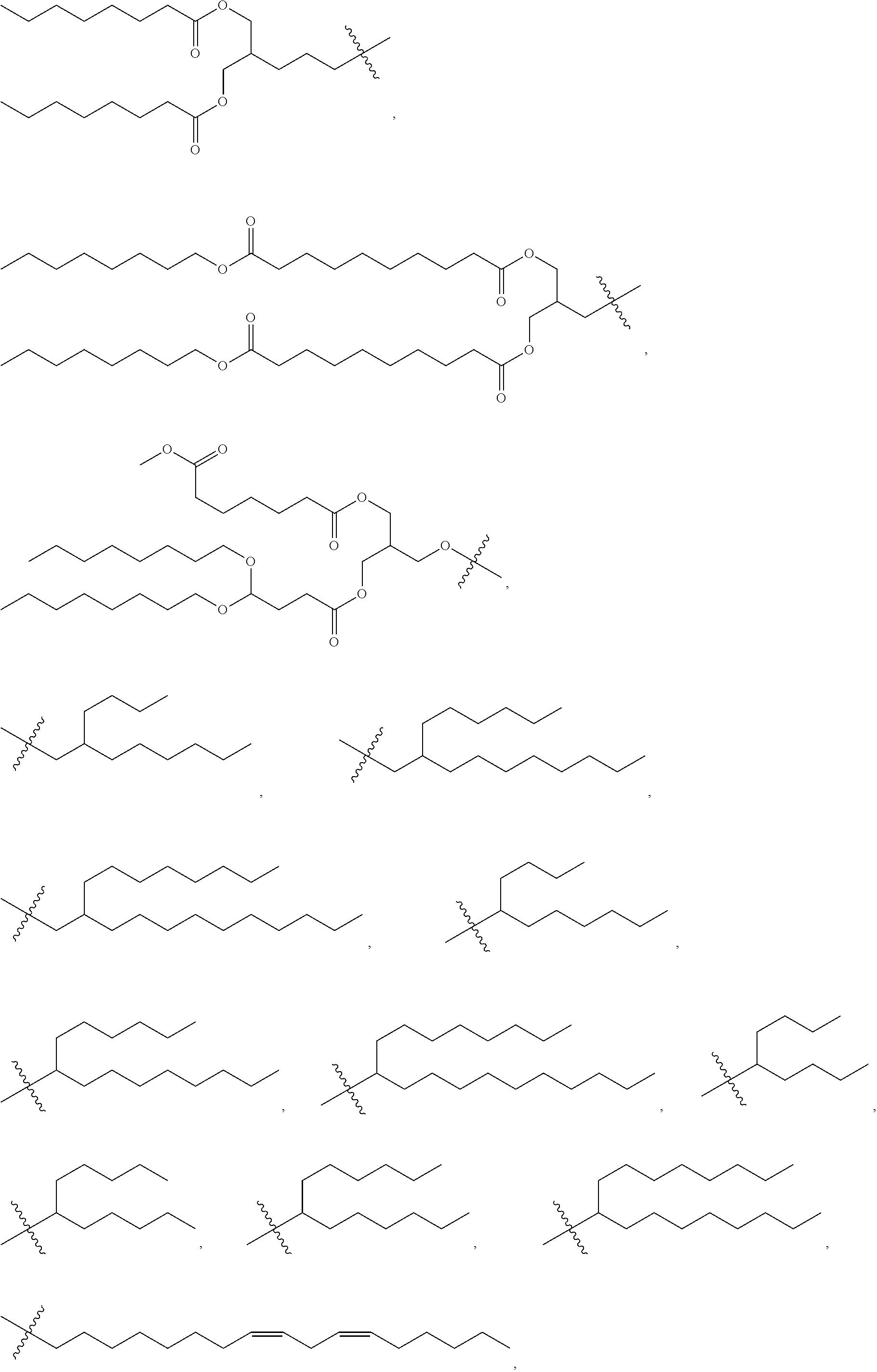

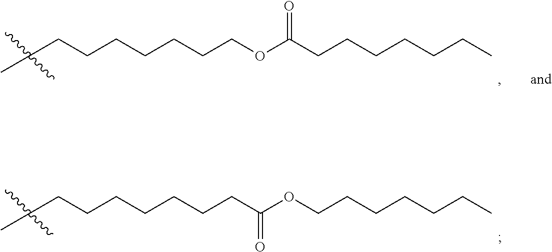 [SMILES: *CCCCCCCOC(=O)CCCCCCC ; *CCCCCCCC(=O)OCCCCCCC]

and

R₃ is selected from a group consisting of:

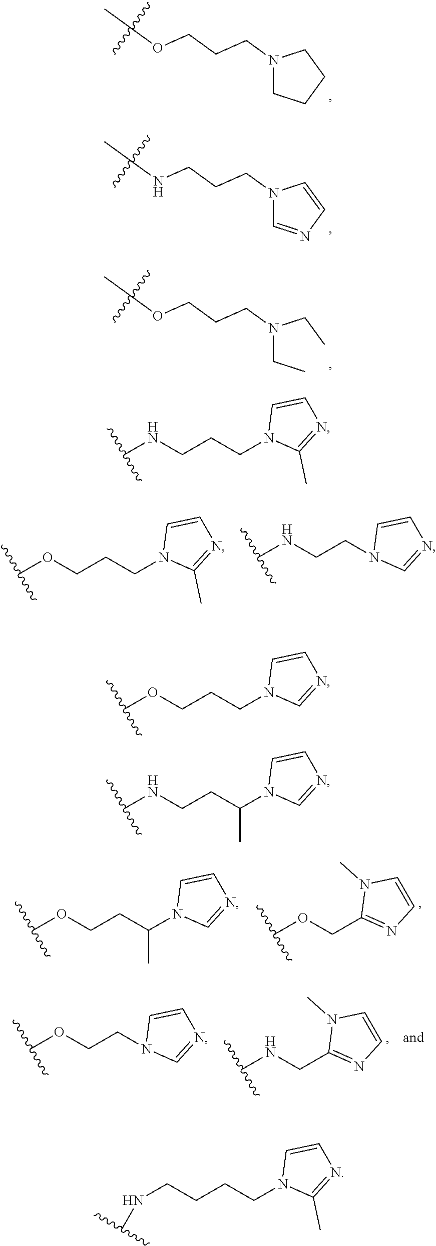

In some embodiments, the ionizable lipid is selected from the group consisting of:

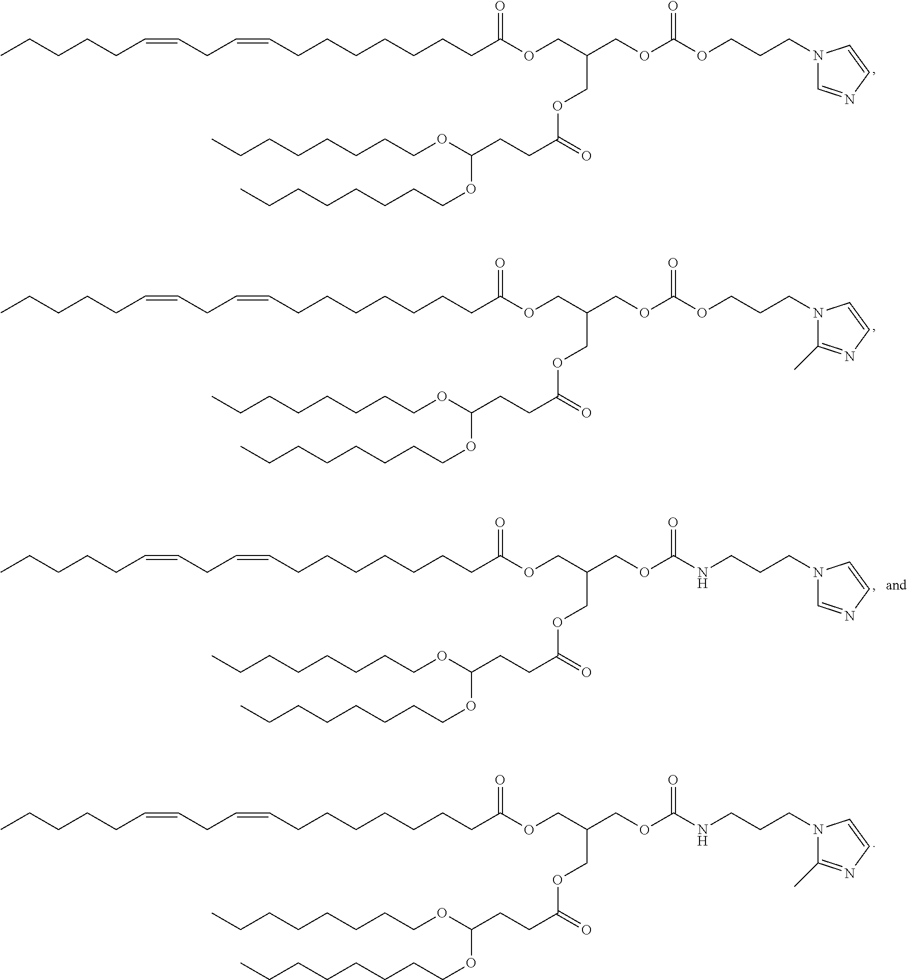 [SMILES: CCCCC/C=C\C/C=C\CCCCCCCC(=O)OCC(COC(=O)CCC(OCCCCCCCC)OCCCCCCCC)COC(=O)OCCCn1ccnc1 ; CCCCC/C=C\C/C=C\CCCCCCCC(=O)OCC(COC(=O)CCC(OCCCCCCCC)OCCCCCCCC)COC(=O)OCCCn1ccnc1C ; CCCCC/C=C\C/C=C\CCCCCCCC(=O)OCC(COC(=O)CCC(OCCCCCCCC)OCCCCCCCC)COC(=O)NCCCn1ccnc1 ; CCCCC/C=C\C/C=C\CCCCCCCC(=O)OCC(COC(=O)CCC(OCCCCCCCC)OCCCCCCCC)COC(=O)NCCCn1ccnc1C]

In another aspect, the present application provides a pharmaceutical composition comprising: a circular RNA polynucleotide, and a transfer vehicle comprising an ionizable lipid represented by Formula (3):

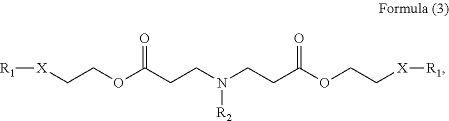

wherein:

X is selected from —O—, —S—, or —OC(O)—*, wherein * indicates the attachment point to R₁;

R₁ is selected from a group consisting of:

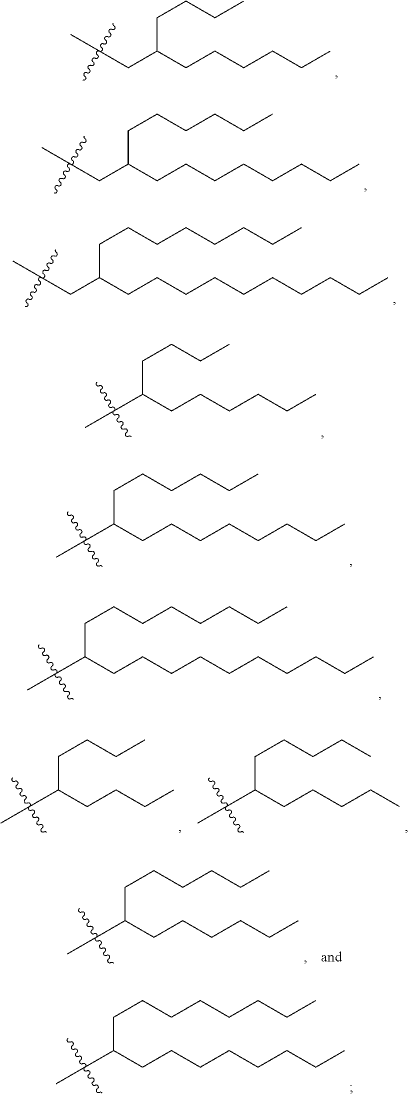

and

R₂ is selected from a group consisting of:

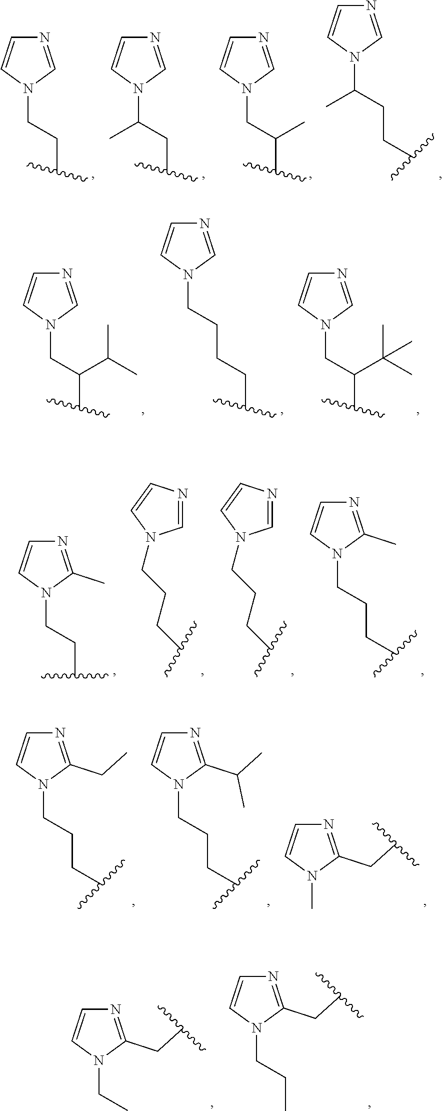

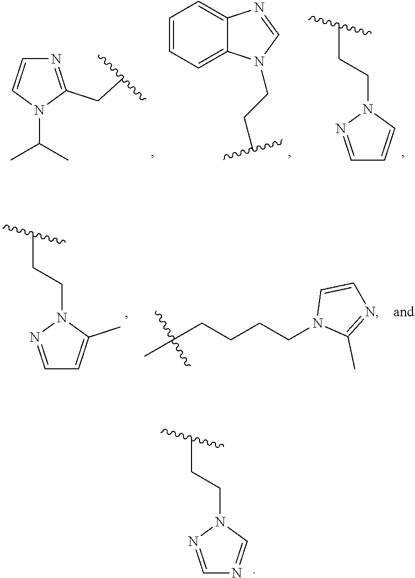

In some embodiments, the ionizable lipid of Formula (3) is represented by Formula (3-1), Formula (3-2), or Formula (3-3):

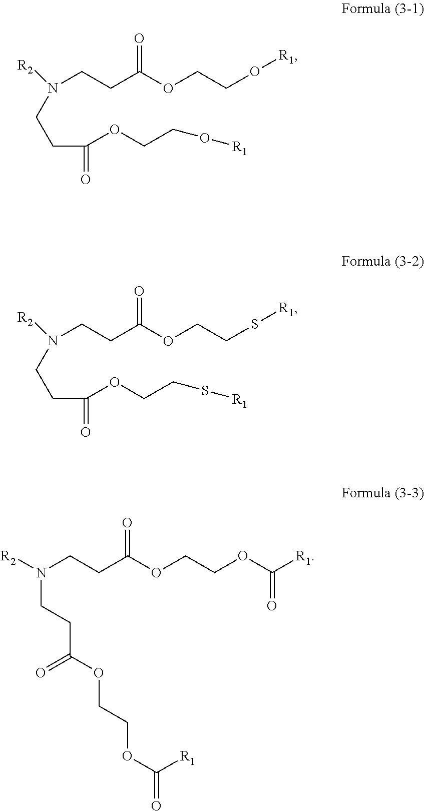

In some embodiments, the ionizable lipid is selected from the group consisting of:

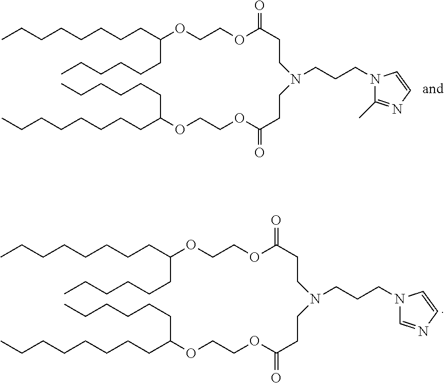 [SMILES: CCCCCCCCC(CCCCCC)OCCOC(=O)CCN(CCCn1ccnc1C)CCC(=O)OCCOC(CCCCCC)CCCCCCCC ; CCCCCCCCC(CCCCCC)OCCOC(=O)CCN(CCCn1ccnc1)CCC(=O)OCCOC(CCCCCC)CCCCCCCC]

In another aspect, the present application provides a pharmaceutical composition comprising: a circular RNA polynucleotide, and a transfer vehicle comprising an ionizable lipid represented by Formula (4):

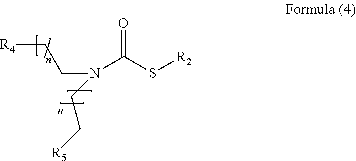

wherein: each n is independently an integer from 2-15; and R₂ is defined in Formula (1).

In another aspect, the present application provides a pharmaceutical composition comprising: a circular RNA polynucleotide, and a transfer vehicle comprising an ionizable lipid represented by Formula (6):

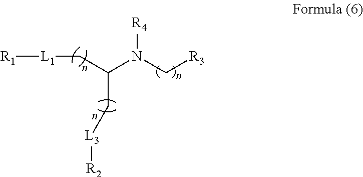

wherein:

each n is independently an integer from 0-15;

L₁ and L₃ are each independently —OC(O)—* or —C(O)O—*, wherein “*” indicates the attachment point to R₁ or R₃;

R₁ and R₂ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkyl sulfoxidealkyl, alkylsulfonyl, and alkylsulfonealkyl;

R₃ is selected from a group consisting of:

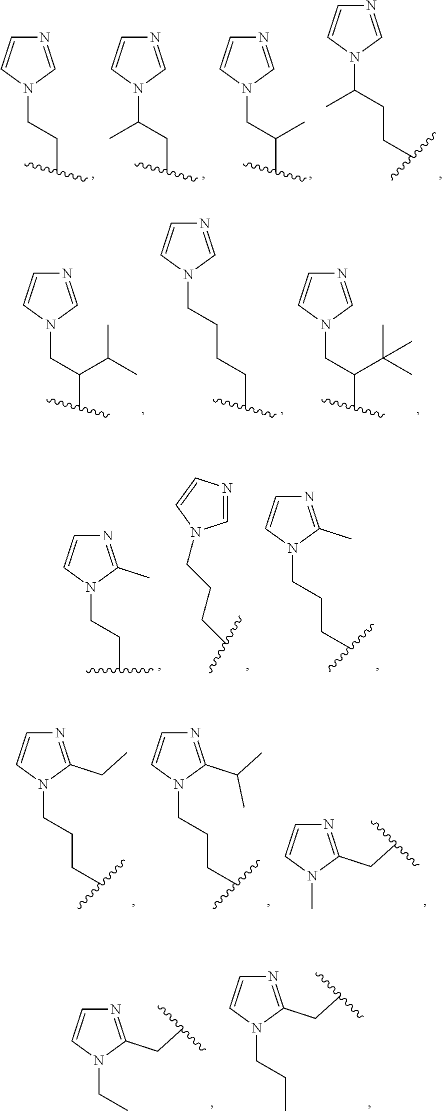

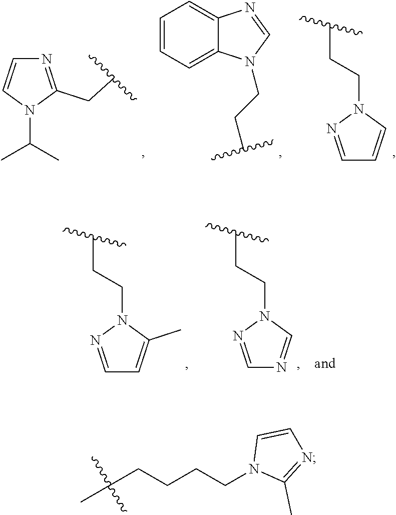

and

R₄ is a linear or branched C₁-C₁₅ alkyl or C₁-C₁₅ alkenyl.

In some embodiments, R₁ and R₂ are each independently selected from a group consisting of:

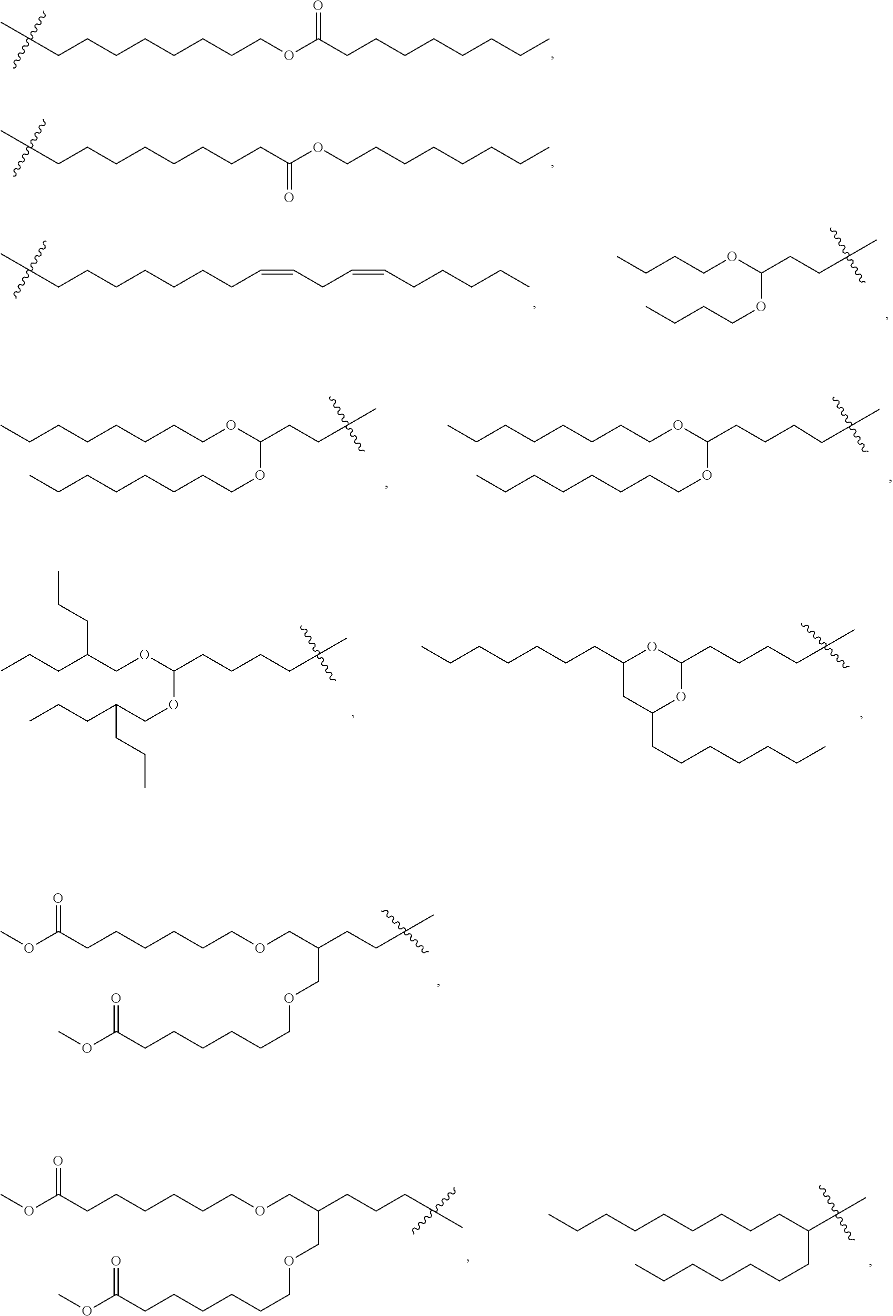

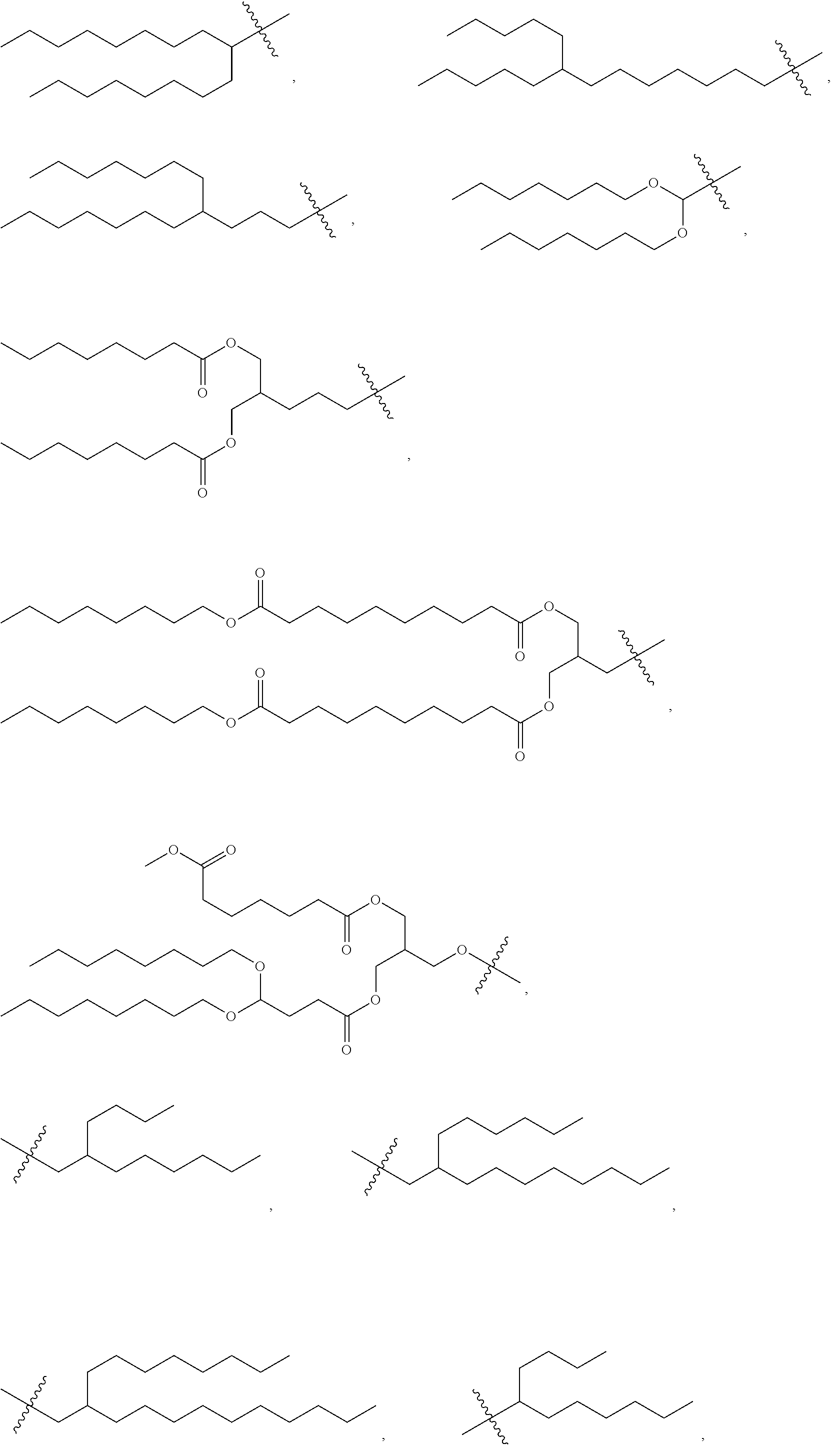

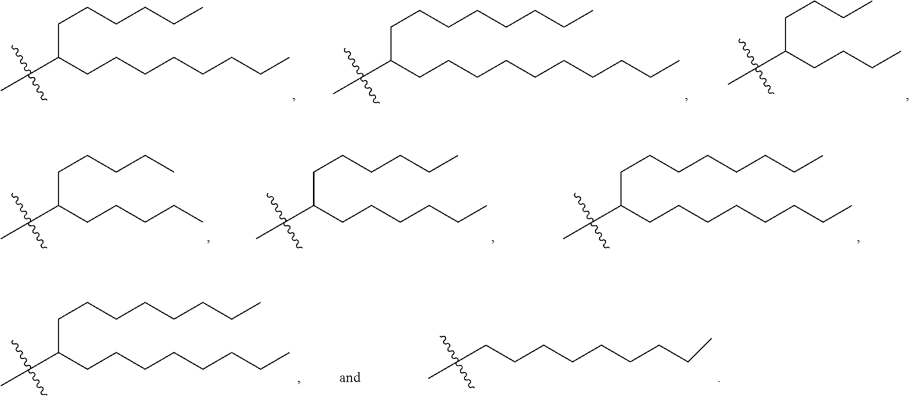

In some embodiments, R₁ and R₂ are the same. In some embodiments, R₁ and R₂ are different.

In some embodiments, the ionizable lipid is selected from the group consisting of:

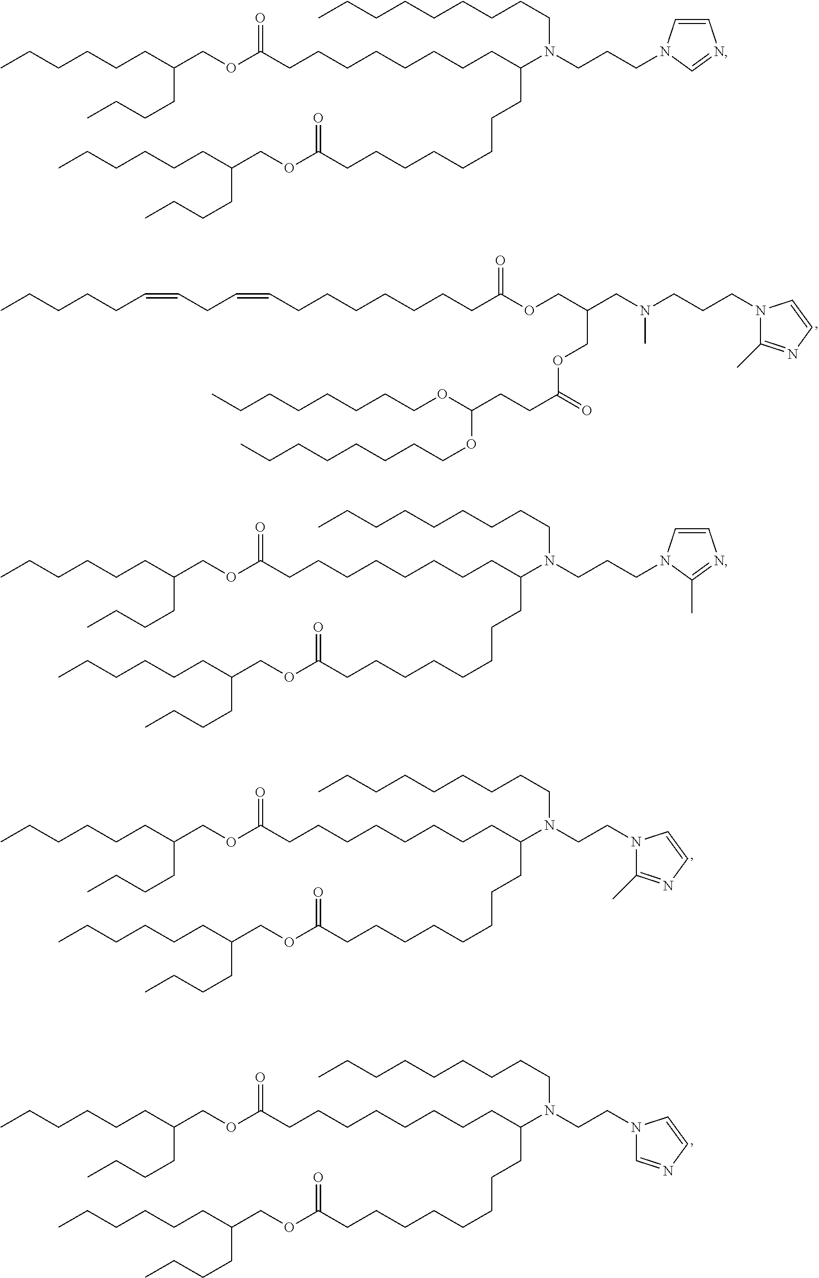 [SMILES: CCCCCCCCCN(CCCn1ccnc1)C(CCCCCCCCC(=O)OCC(CCCC)CCCCCC)CCCCCCCCC(=O)OCC(CCCC)CCCCCC ; CCCCC/C=C\C/C=C\CCCCCCCC(=O)OCC(COC(=O)CCC(OCCCCCCCC)OCCCCCCCC)CN(C)CCCn1ccnc1C ; CCCCCCCCCN(CCCn1ccnc1C)C(CCCCCCCCC(=O)OCC(CCCC)CCCCCC)CCCCCCCCC(=O)OCC(CCCC)CCCCCC ; CCCCCCCCCN(CCn1ccnc1C)C(CCCCCCCCC(=O)OCC(CCCC)CCCCCC)CCCCCCCCC(=O)OCC(CCCC)CCCCCC ; CCCCCCCCCN(CCn1ccnc1)C(CCCCCCCCC(=O)OCC(CCCC)CCCCCC)CCCCCCCCC(=O)OCC(CCCC)CCCCCC]

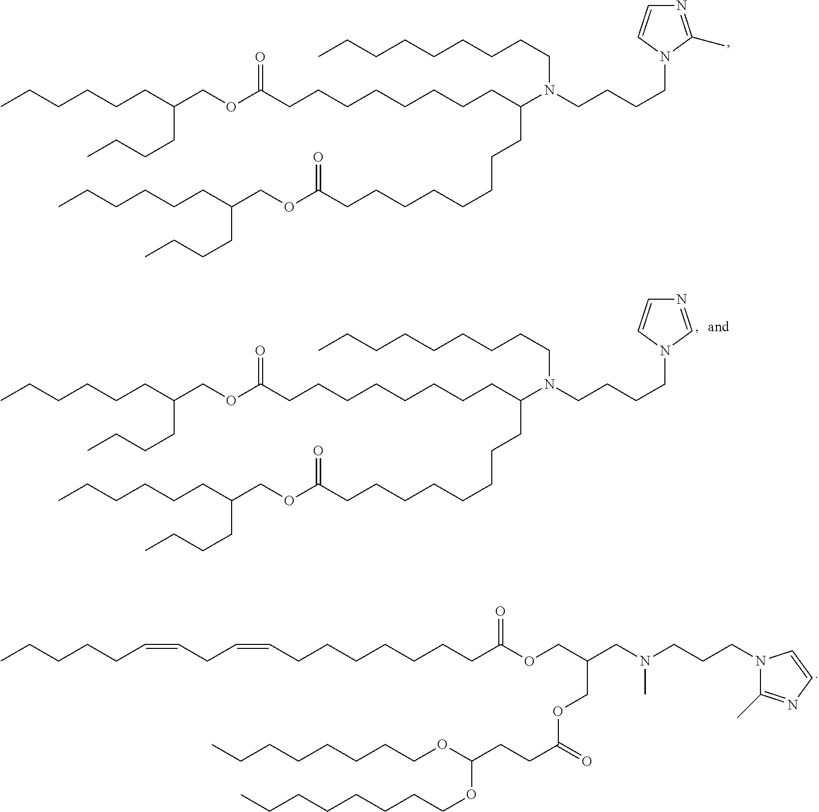

In another aspect, the present application provides a pharmaceutical composition comprising: a circular RNA polynucleotide, and a transfer vehicle comprising an ionizable lipid selected from Table 10a.

In some embodiments of pharmaceutical compositions provided herein, the circular RNA polynucleotide is encapsulated in the transfer vehicle. In some embodiments, the circular RNA polynucleotide is encapsulated in the transfer vehicle with an encapsulation efficiency of at least 80%.

In some embodiments, the circular RNA comprises a first expression sequence. In some embodiments, the first expression sequence encodes a therapeutic protein. In some embodiments, the first expression sequence encodes a cytokine or a functional fragment thereof. In some embodiments, the first expression sequence encodes a transcription factor. In some embodiments, the first expression sequence encodes an immune checkpoint inhibitor. In some embodiments, the first expression sequence encodes a chimeric antigen receptor.

In some embodiments, the circular RNA polynucleotide further comprises a second expression sequence. In some embodiments, the circular RNA polynucleotide further comprises an internal ribosome entry site (IRES).

In some embodiments, the first and second expression sequences are separated by a ribosomal skipping element or a nucleotide sequence encoding a protease cleavage site. In some embodiments, the first expression sequence encodes a first T-cell receptor (TCR) chain and the second expression sequence encodes a second TCR chain.

In some embodiments, the circular RNA polynucleotide comprises one or more microRNA binding sites. the microRNA binding site is recognized by a microRNA expressed in the liver. In some embodiments, the microRNA binding site is recognized by miR-122.

In some embodiments, the circular RNA polynucleotide comprises a first IRES associated with greater protein expression in a human immune cell than in a reference human cell. In some embodiments, the human immune cell is a T cell, an NK cell, an NKT cell, a macrophage, or a neutrophil. In some embodiments, the reference human cell is a hepatic cell.

In some embodiments, the circular RNA polynucleotide comprises, in the following order: a) a post-splicing intron fragment of a 3′ group I intron fragment, b) an IRES, c) an expression sequence, and d) a post-splicing intron fragment of a 5′ group I intron fragment. In some embodiments, the circular RNA polynucleotide comprises. In some embodiments, the circular RNA polynucleotide comprises a first spacer before the post-splicing intron fragment of the 3′ group I intron fragment, and a second spacer after the post-splicing intron fragment of the 5′ group I intron fragment. In some embodiments, the first and second spacers each have a length of about 10 to about 60 nucleotides.

In some embodiments, the circular RNA polynucleotide is made via circularization of a RNA polynucleotide comprising, in the following order: a 3′ group I intron fragment, an IRES, an expression sequence, and a 5′ group I intron fragment.

In some embodiments, the circular RNA polynucleotide is made via circularization of a RNA polynucleotide comprising, in the following order: a 5′ external duplex forming region, a 3′ group I intron fragment, a 5′ internal spacer optionally comprising a 5′ internal duplex forming region, an IRES, an expression sequence, a 3′ internal spacer optionally comprising a 3′ internal duplex forming region, a 5′ group I intron fragment, and a 3′ external duplex forming region.

In some embodiments, the circular RNA polynucleotide is made via circularization of a RNA polynucleotide comprising, in the following order: a 5′ external duplex forming region, a 5′ external spacer, a 3′ group I intron fragment, a 5′ internal spacer optionally comprising a 5′ internal duplex forming region, an IRES, an expression sequence, a 3′ internal spacer optionally comprising a 3′ internal duplex forming region, a 5′ group I intron fragment, a 3′ external spacer, and a 3′ external duplex forming region.

In some embodiments, the circular RNA polynucleotide is made via circularization of a RNA polynucleotide comprising, in the following order: a 3′ group I intron fragment, a 5′ internal spacer comprising a 5′ internal duplex forming region, an IRES, an expression sequence, a 3′ internal spacer comprising a 3′ internal duplex forming region, and a 5′ group I intron fragment.

In some embodiments, the circular RNA polynucleotide is made via circularization of a RNA polynucleotide comprising, in the following order: a 5′ external duplex forming region, a 5′ external spacer, a 3′ group I intron fragment, a 5′ internal spacer comprising a 5′ internal duplex forming region, an IRES, an expression sequence, a 3′ internal spacer comprising a 3′ internal duplex forming region, a 5′ group I intron fragment, a 3′ external spacer, and a 3′ external duplex forming region.

In some embodiments, the circular RNA polynucleotide is made via circularization of a RNA polynucleotide comprising, in the following order: a first polyA sequence, a 5′ external duplex forming region, a 5′ external spacer, a 3′ group I intron fragment, a 5′ internal spacer comprising a 5′ internal duplex forming region, an IRES, an expression sequence, a 3′ internal spacer comprising a 3′ internal duplex forming region, a 5′ group I intron fragment, a 3′ external spacer, a 3′ external duplex forming region, and a second polyA sequence.

In some embodiments, the circular RNA polynucleotide is made via circularization of a RNA polynucleotide comprising, in the following order: a first polyA sequence, a 5′ external spacer, a 3′ group I intron fragment, a 5′ internal spacer comprising a 5′ internal duplex forming region, an IRES, an expression sequence, a 3′ internal spacer comprising a 3′ internal duplex forming region, a 5′ group I intron fragment, a 3′ external spacer, and a second polyA sequence.

In some embodiments, the circular RNA polynucleotide is made via circularization of a RNA polynucleotide comprising, in the following order: a first polyA sequence, a 5′ external spacer, a 3′ group I intron fragment, a 5′ internal spacer comprising a 5′ internal duplex forming region, an IRES, an expression sequence, a stop condon, a 3′ internal spacer comprising a 3′ internal duplex forming region, a 5′ group I intron fragment, a 3′ external spacer, and a second polyA sequence.

In some embodiments, at least one of the 3′ or 5′ internal or external spacers has a length of about 8 to about 60 nucleotides. In some embodiments, the 3′ and 5′ external duplex forming regions each has a length of about 10-50 nucleotides. In some embodiments, the 3′ and 5′ internal duplex forming regions each has a length of about 6-30 nucleotides.

In some embodiments, the IRES is selected from Table 17, or is a functional fragment or variant thereof. In some embodiments, the IRES has a sequence of an IRES from Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, Foot and mouth disease virus, Human enterovirus 71, Equine rhinitis virus, Ectropis obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human eIF4G, Mouse NDST4L, Human LEF1, Mouse HIF1 alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SH1, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picorna-like Virus, CRPV, Apodemus Agrarius Picornavirus, Caprine Kobuvirus, Parabovirus, Salivirus A BNS, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24, or an aptamer to eIF4G.

In some embodiments, the first and second polyA sequences each have a length of about 15-50 nt. In some embodiments, the first and second polyA sequences each have a length of about 20-25 nt.

In some embodiments, the circular RNA polynucleotide contains at least about 80%, at least about 90%, at least about 95%, or at least about 99% naturally occurring nucleotides. In some embodiments, the circular RNA polynucleotide consists of naturally occurring nucleotides.

In some embodiments, the expression sequence is codon optimized. In some embodiments, the circular RNA polynucleotide is optimized to lack at least one microRNA binding site present in an equivalent pre-optimized polynucleotide. In some embodiments, the circular RNA polynucleotide is optimized to lack at least one microRNA binding site capable of binding to a microRNA present in a cell within which the circular RNA polynucleotide is expressed. In some embodiments, the circular RNA polynucleotide is optimized to lack at least one endonuclease susceptible site present in an equivalent pre-optimized polynucleotide. In some embodiments, the circular RNA polynucleotide is optimized to lack at least one endonuclease susceptible site capable of being cleaved by an endonuclease present in a cell within which the endonuclease is expressed. In some embodiments, the circular RNA polynucleotide is optimized to lack at least one RNA editing susceptible site present in an equivalent pre-optimized polynucleotide.

In some embodiments, the circular RNA polynucleotide is from about 100 nt to about 10,000 nt in length. In some embodiments, the circular RNA polynucleotide is from about 100 nt to about 15,000 nt in length. In some embodiments, the circular RNA is more compact than a reference linear RNA polynucleotide having the same expression sequence as the circular RNA polynucleotide.

In some embodiments, the pharmaceutical composition has a duration of therapeutic effect in a human cell greater than or equal to that of a composition comprising a reference linear RNA polynucleotide having the same expression sequence as the circular RNA polynucleotide. In some embodiments, the reference linear RNA polynucleotide is a linear, unmodified or nucleoside-modified, fully-processed mRNA comprising a cap1 structure and a polyA tail at least 80 nt in length.

In some embodiments, the pharmaceutical composition has a duration of therapeutic effect in vivo in humans greater than that of a composition comprising a reference linear RNA polynucleotide having the same expression sequence as the circular RNA polynucleotide. In some embodiments, the pharmaceutical composition has an duration of therapeutic effect in vivo in humans of at least about 10, at least about 20, at least about 30, at least about 40, at least about 50, at least about 60, at least about 70, at least about 80, at least about 90, or at least about 100 hours.

In some embodiments, the pharmaceutical composition has a functional half-life in a human cell greater than or equal to that of a pre-determined threshold value. In some embodiments, the pharmaceutical composition has a functional half-life in vivo in humans greater than that of a pre-determined threshold value. In some embodiments, the functional half-life is determined by a functional protein assay. In some embodiments, the functional protein assay is an in vitro luciferase assay. In some embodiments, the functional protein assay comprises measuring levels of protein encoded by the expression sequence of the circular RNA polynucleotide in a patient serum or tissue sample. In some embodiments, wherein the pre-determined threshold value is the functional half-life of a reference linear RNA polynucleotide comprising the same expression sequence as the circular RNA polynucleotide. In some embodiments, the pharmaceutical composition has a functional half-life of at least about 20 hours.

In some embodiments, the pharmaceutic composition comprises a structural lipid and a PEG-modified lipid. In some embodiments, the structural lipid binds to C1q and/or promotes the binding of the transfer vehicle comprising said lipid to C1q compared to a control transfer vehicle lacking the structural lipid and/or increases uptake of C1q-bound transfer vehicle into an immune cell compared to a control transfer vehicle lacking the structural lipid. In some embodiments, the immune cell is a T cell, an NK cell, an NKT cell, a macrophage, or a neutrophil.

In some embodiments, the structural lipid is cholesterol. In some embodiments, the structural lipid is beta-sitosterol. In some embodiments, the structural lipid is not beta-sitosterol.

In some embodiments, the PEG-modified lipid is DSPE-PEG, DMG-PEG, or PEG-1. In some embodiments, the PEG-modified lipid is DSPE-PEG(2000).

In some embodiments, the pharmaceutic composition further comprises a helper lipid. In some embodiments, the helper lipid is DSPC or DOPE.

In some embodiments, the pharmaceutic composition comprises DOPE, cholesterol, and DSPE-PEG.

In some embodiments, the transfer vehicle comprises about 0.5% to about 4% PEG-modified lipids by molar ratio. In some embodiments, the transfer vehicle comprises about 1% to about 2% PEG-modified lipids by molar ratio.

In some embodiments, the transfer vehicle comprises

a. an ionizable lipid selected from

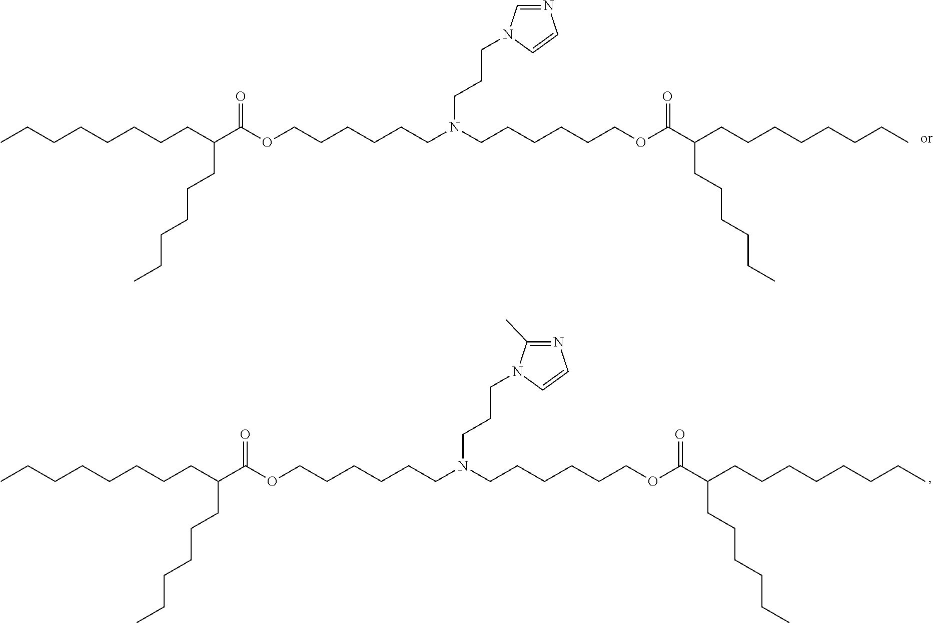

or a mixture thereof,

b. a helper lipid selected from DOPE or DSPC,

c. cholesterol, and

d. a PEG-lipid selected from DSPE-PEG(2000) or DMG-PEG(2000).

In some embodiments, the transfer vehicle comprises

a. an ionizable lipid selected from

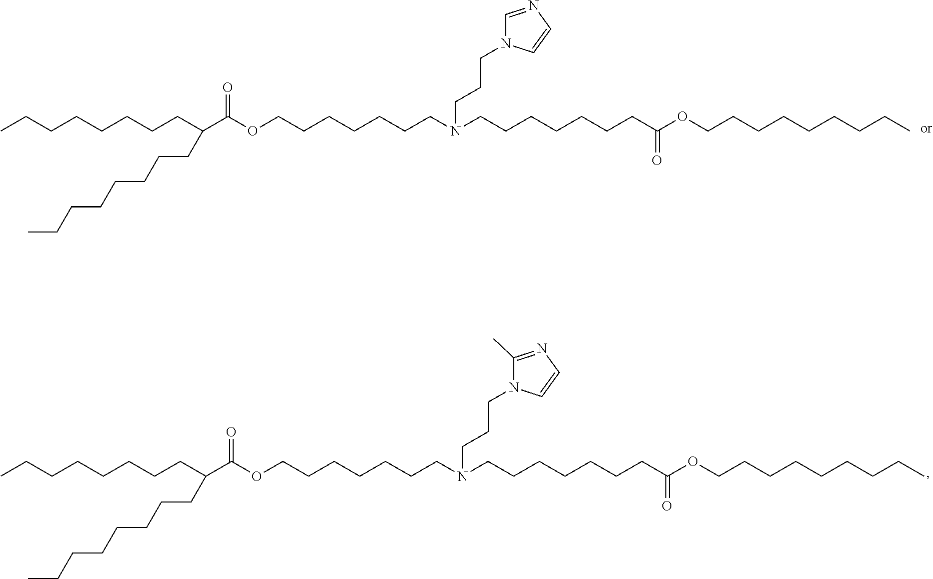

or a mixture thereof,

b. a helper lipid selected from DOPE or DSPC,

c. cholesterol, and

d. a PEG-lipid selected from DSPE-PEG(2000) or DMG-PEG(2000).

In some embodiments, the transfer vehicle comprises

a. an ionizable lipid selected from

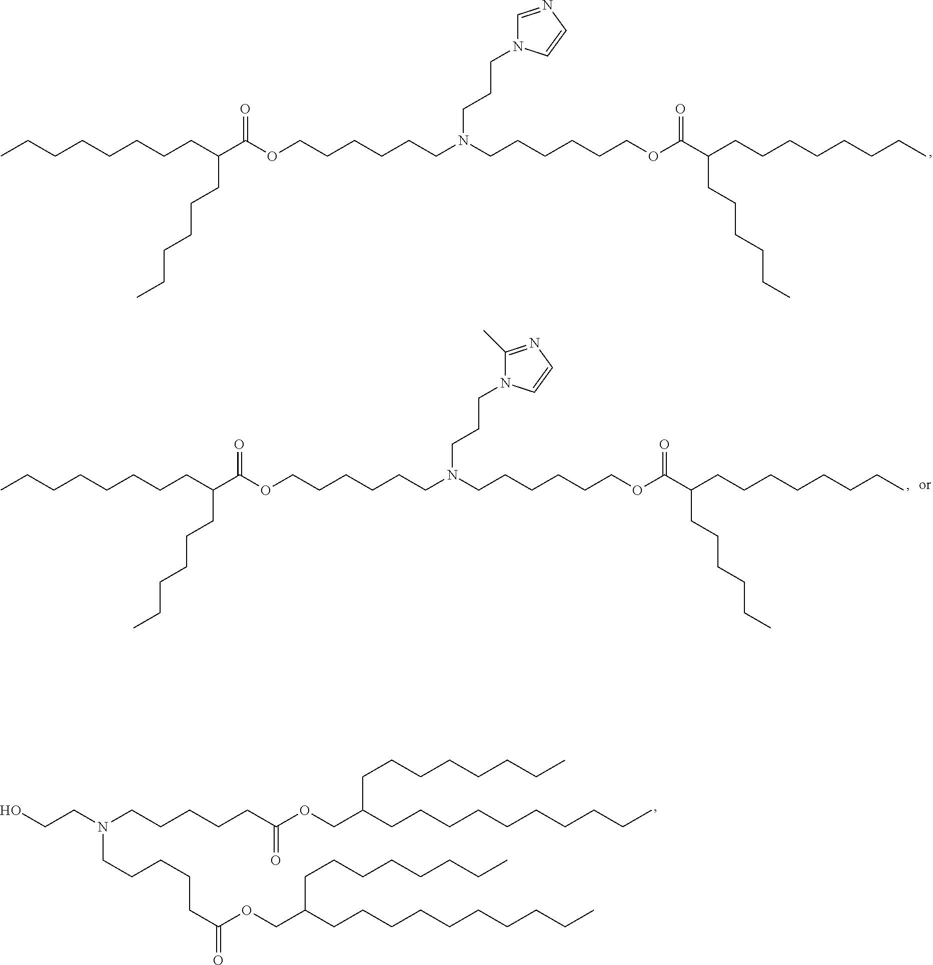

or a mixture thereof,

b. a helper lipid selected from DOPE or DSPC,

c. cholesterol, and

d. a PEH-lipid of DMG-PEG(2000).

In some embodiments, the transfer vehicle comprises

a. an ionizable lipid selected from

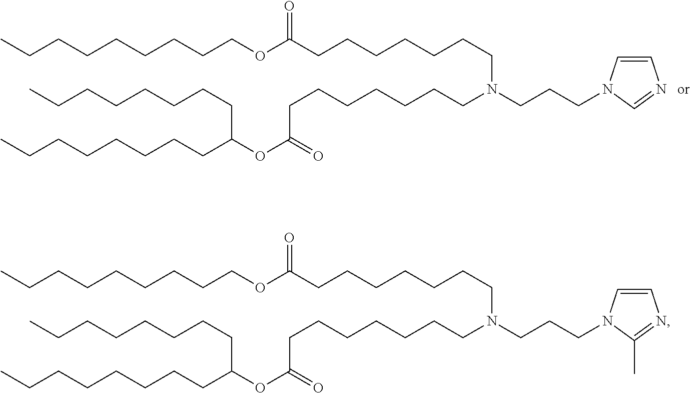 [SMILES: CCCCCCCCCOC(=O)CCCCCCCN(CCCCCCCC(=O)OC(CCCCCCCC)CCCCCCCC)CCCn1ccnc1 ; CCCCCCCCCOC(=O)CCCCCCCN(CCCCCCCC(=O)OC(CCCCCCCC)CCCCCCCC)CCCn1ccnc1C]

or a mixture thereof,

b. a helper lipid selected from DOPE or DSPC,

c. cholesterol, and

d. a PEG-lipid selected from DSPE-PEG(2000), DMG-PEG(2000), or C₁₄-PEG(2000).

In some embodiments, the transfer vehicle comprises

a. an ionizable lipid selected from

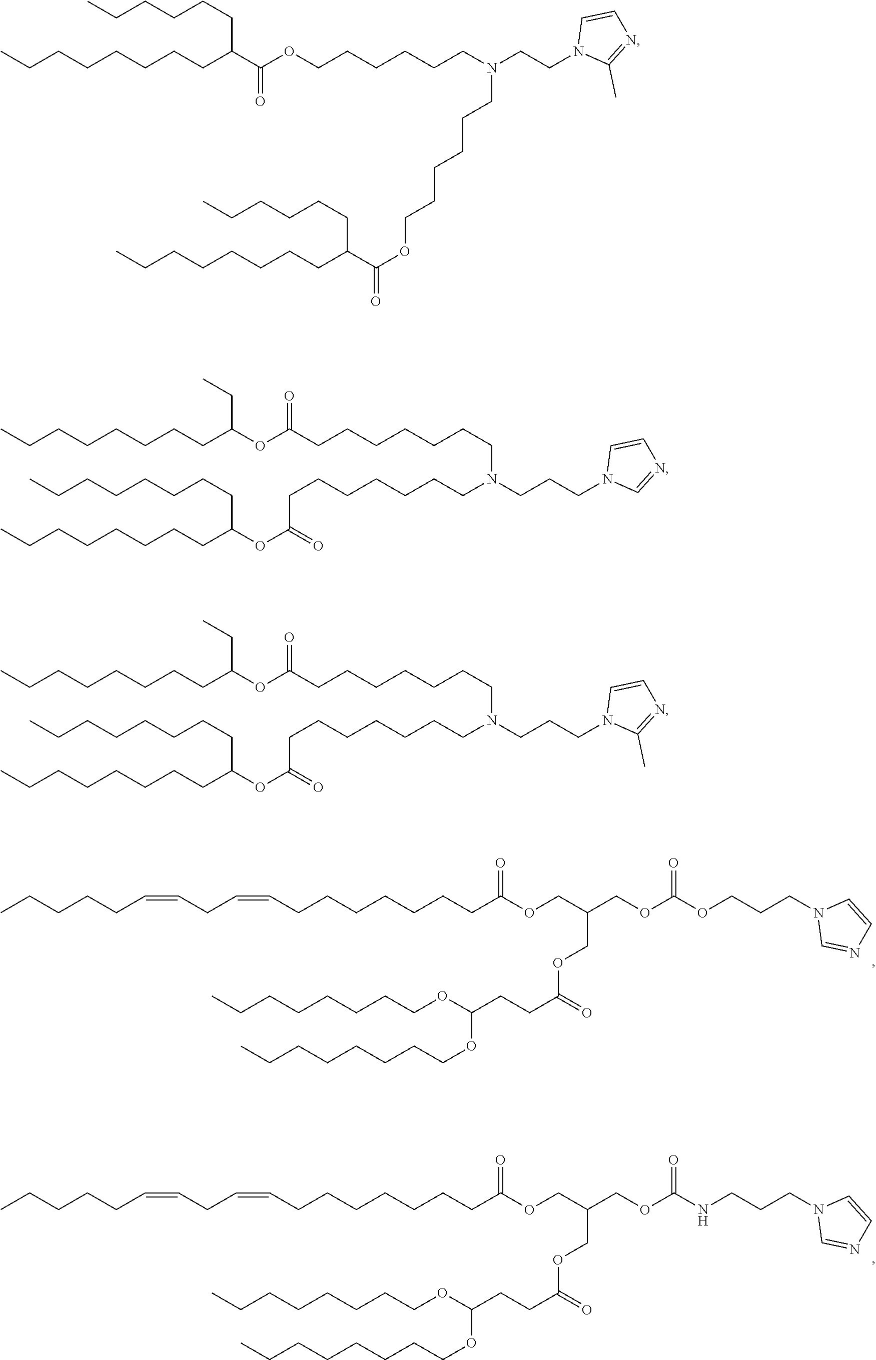 [SMILES: CCCCCCCCC(CCCCCC)C(=O)OCCCCCCN(CCCCCCOC(=O)C(CCCCCC)CCCCCCCC)CCn1ccnc1C ; CCCCCCCCC(CC)OC(=O)CCCCCCCN(CCCCCCCC(=O)OC(CCCCCCCC)CCCCCCCC)CCCn1ccnc1 ; CCCCCCCCC(CC)OC(=O)CCCCCCCN(CCCCCCCC(=O)OC(CCCCCCCC)CCCCCCCC)CCCn1ccnc1C ; CCCCC/C=C\C/C=C\CCCCCCCC(=O)OCC(COC(=O)CCC(OCCCCCCCC)OCCCCCCCC)COC(=O)OCCCn1ccnc1 ; CCCCC/C=C\C/C=C\CCCCCCCC(=O)OCC(COC(=O)CCC(OCCCCCCCC)OCCCCCCCC)COC(=O)NCCCn1ccnc1]

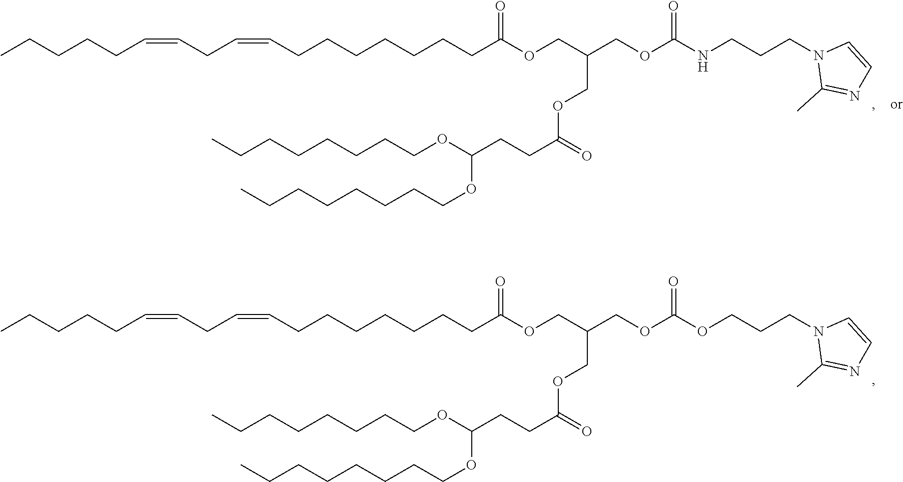 [SMILES: CCCCC/C=C\C/C=C\CCCCCCCC(=O)OCC(COC(=O)CCC(OCCCCCCCC)OCCCCCCCC)COC(=O)NCCCn1ccnc1C ; CCCCC/C=C\C/C=C\CCCCCCCC(=O)OCC(COC(=O)CCC(OCCCCCCCC)OCCCCCCCC)COC(=O)OCCCn1ccnc1C]

or a mixture thereof,

b. a helper lipid selected from DOPE or DSPC,

c. cholesterol, and

d. a PEH-lipid of DMG-PEG(2000).

In some embodiments, the transfer vehicle comprises

a. an ionizable lipid selected from

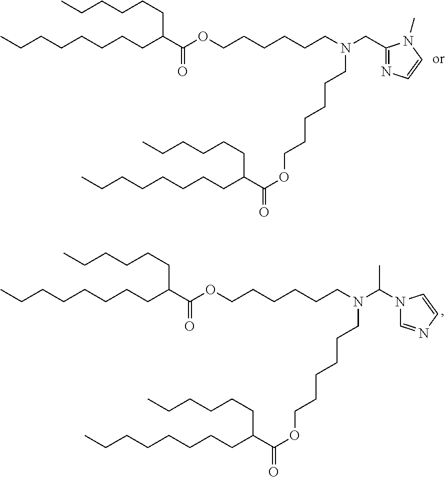 [SMILES: CCCCCCCCC(CCCCCC)C(=O)OCCCCCCN(CCCCCCOC(=O)C(CCCCCC)CCCCCCCC)Cc1nccn1C ; CCCCCCCCC(CCCCCC)C(=O)OCCCCCCN(CCCCCCOC(=O)C(CCCCCC)CCCCCCCC)C(C)n1ccnc1]

or a mixture thereof,

b. a helper lipid selected from DOPE or DSPC,

c. cholesterol, and

d. a PEH-lipid selected from DSPE-PEG(2000) or DMG-PEG(2000).

In some embodiments, the transfer vehicle comprises

a. an ionizable lipid selected from

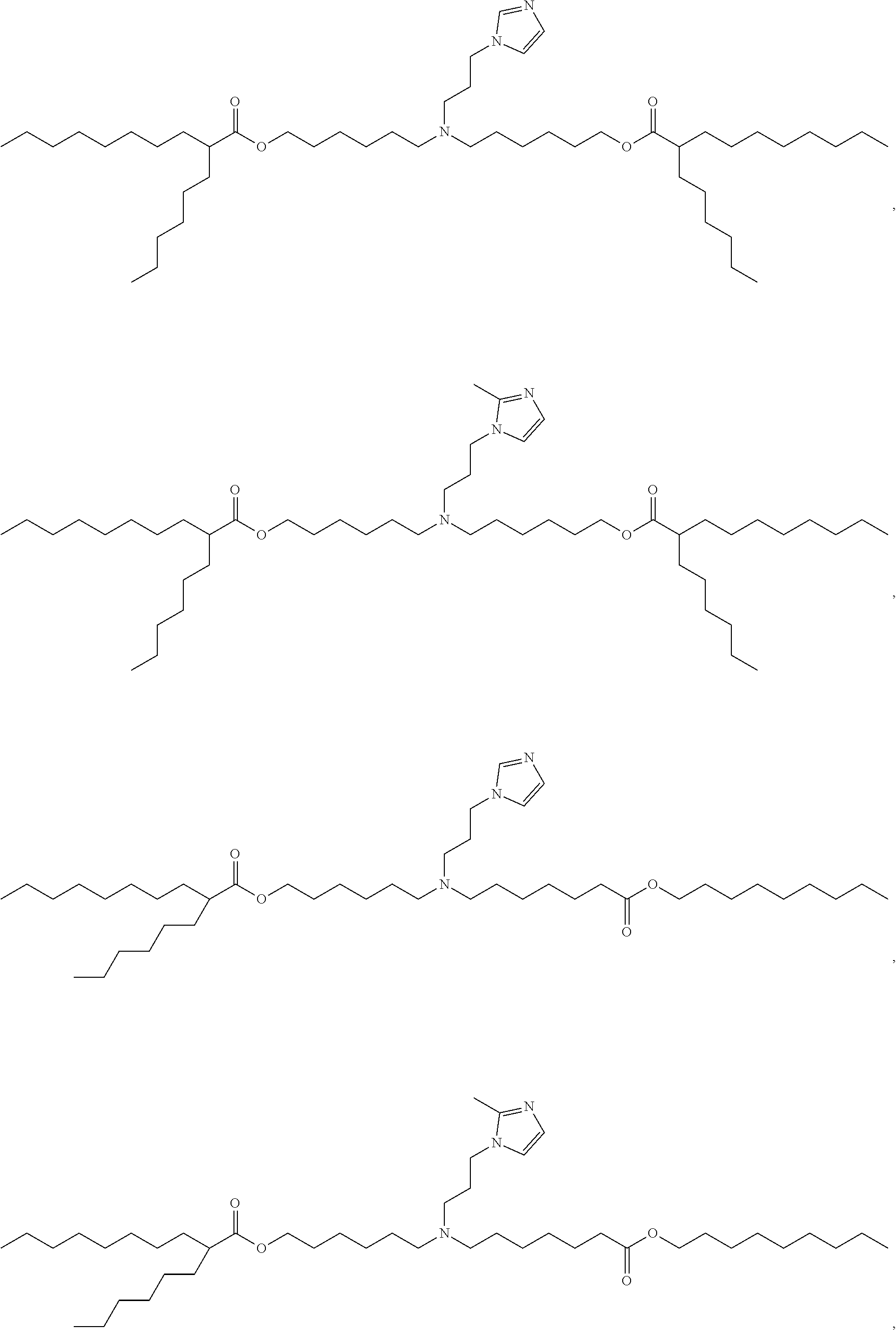

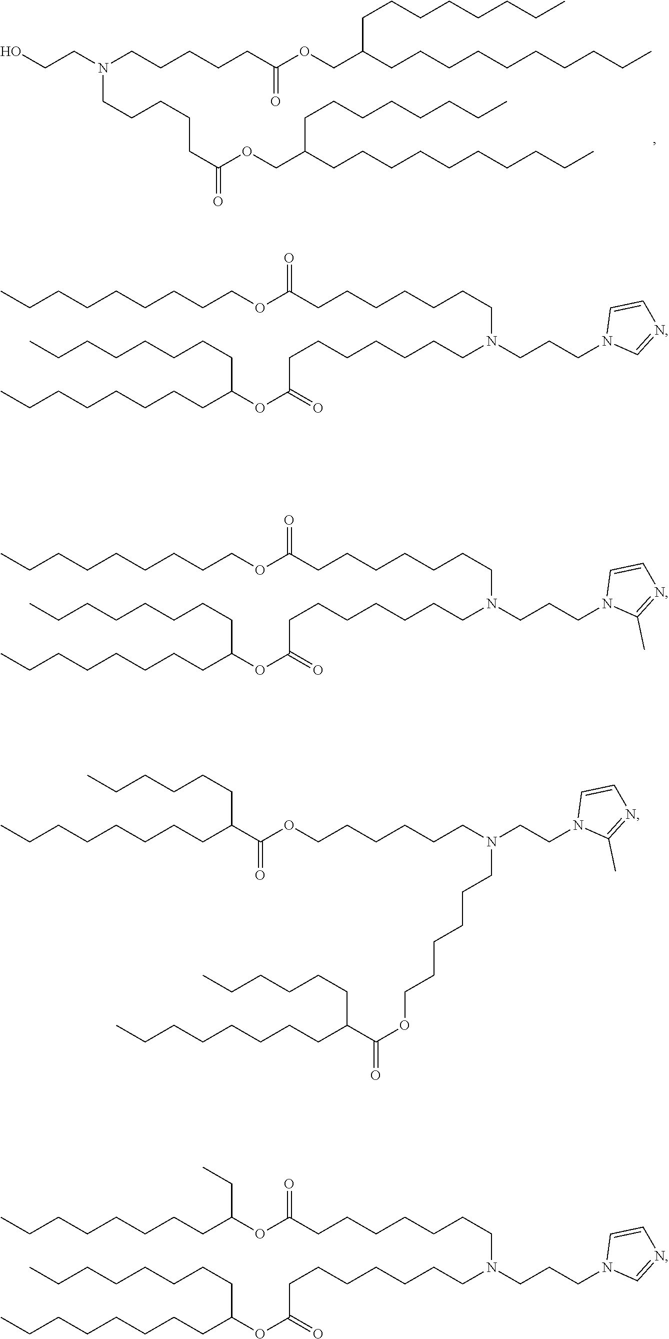

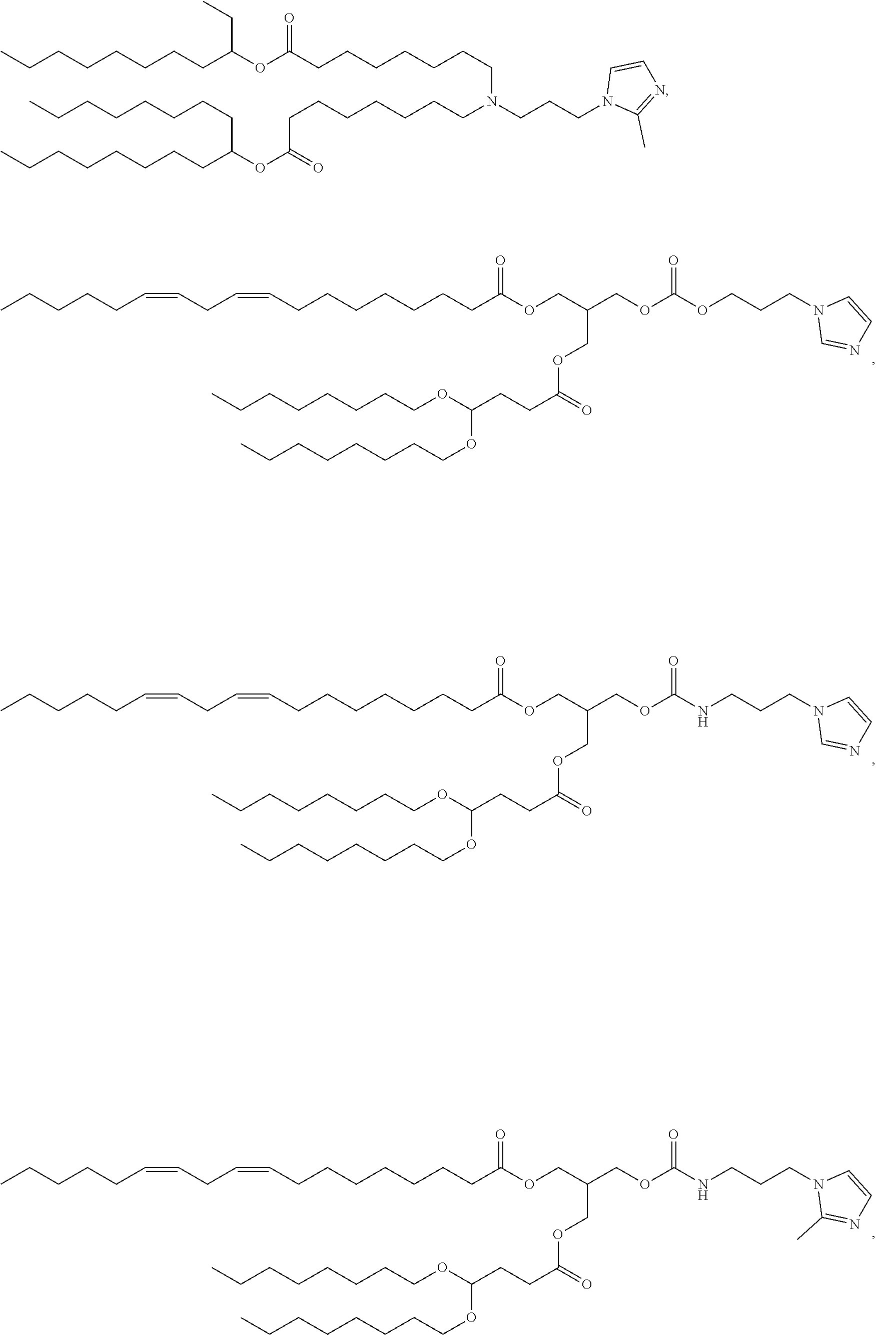 [SMILES: CCCCCCCCC(CC)OC(=O)CCCCCCCN(CCCCCCCC(=O)OC(CCCCCCCC)CCCCCCCC)CCCn1ccnc1C ; CCCCC/C=C\C/C=C\CCCCCCCC(=O)OCC(COC(=O)CCC(OCCCCCCCC)OCCCCCCCC)COC(=O)OCCCn1ccnc1 ; CCCCC/C=C\C/C=C\CCCCCCCC(=O)OCC(COC(=O)CCC(OCCCCCCCC)OCCCCCCCC)COC(=O)NCCCn1ccnc1 ; CCCCC/C=C\C/C=C\CCCCCCCC(=O)OCC(COC(=O)CCC(OCCCCCCCC)OCCCCCCCC)COC(=O)NCCCn1ccnc1C]

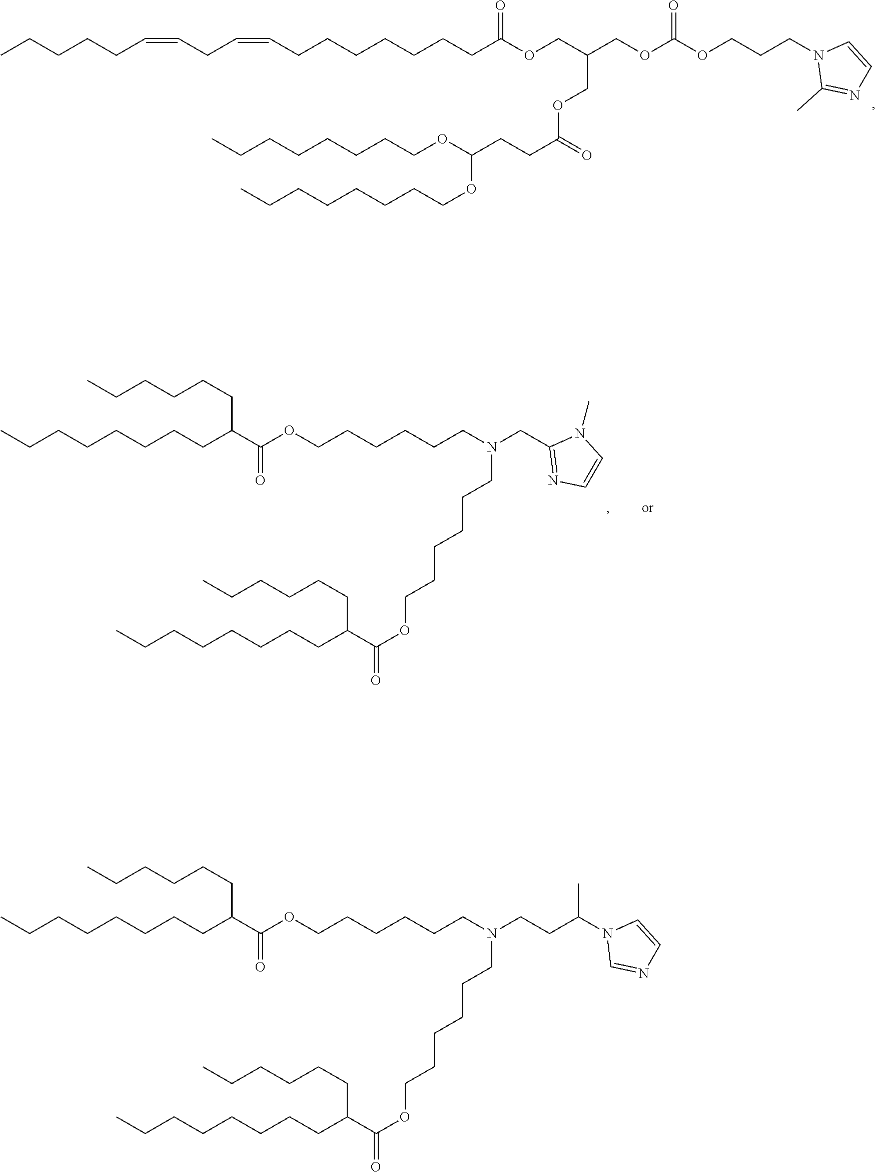 [SMILES: CCCCC/C=C\C/C=C\CCCCCCCC(=O)OCC(COC(=O)CCC(OCCCCCCCC)OCCCCCCCC)COC(=O)OCCCn1ccnc1C ; CCCCCCCCC(CCCCCC)C(=O)OCCCCCCN(CCCCCCOC(=O)C(CCCCCC)CCCCCCCC)Cc1nccn1C ; CCCCCCCCC(CCCCCC)C(=O)OCCCCCCN(CCCCCCOC(=O)C(CCCCCC)CCCCCCCC)CCC(C)n1ccnc1]

or a mixture thereof,

b. a helper lipid selected from DOPE or DSPC,

c. cholesterol, and

d. a PEH-lipid selected from DSPE-PEG(2000), DMG-PEG(2000), or C₁₄-PEG(2000).

In some embodiments, the molar ratio of ionizable lipid:helper lipid:cholesterol:PEG-lipid is 62:4:33:1. In some embodiments, the molar ratio of ionizable lipid:helper lipid:cholesterol:PEG-lipid is 50:10:38.5:1.5. In some embodiments, the molar ration of ionizable lipid:helper lipid:cholesterol:PEG-lipid is 35:16:46.2.5. In some embodiments, the molar ration of ionizable lipid:helper lipid:cholesterol:PEG-lipid is 40:10:40:10.

In some embodiments, the transfer vehicle comprises the helper lipid of DOPE and the PEG-lipid of DMG-PEG(2000), and wherein the molar ratio of ionizable lipid:DOPE:cholesterol:DMG-PEG(2000) is 62:4:33:1. In some embodiments, the transfer vehicle comprises the helper lipid of DOPE and the PEG-lipid of DMG-PEG(2000), and wherein the molar ratio of ionizable lipid:DOPE:cholesterol:DMG-PEG(2000) is 50:10:38.5:1.5. In some embodiments, the transfer vehicle comprises the helper lipid of DOPE and the PEG-lipid of DSPE-PEG(2000), and wherein the molar ratio of ionizable lipid:DOPE:cholesterol:DSPE-PEG(2000) is 62:4:33:1. In some embodiments, the transfer vehicle comprises the helper lipid of DOPE and the PEG-lipid of DSPE-PEG(2000), and wherein the molar ratio of ionizable lipid:DOPE:cholesterol:DSPE-PEG(2000) is 50:10:38.5:1.5.

In some embodiments, the transfer vehicle comprises the helper lipid of DSPC and the PEG-lipid of DMG-PEG(2000), and wherein the molar ratio of ionizable lipid:DSPC:cholesterol:DMG-PEG(2000) is 62:4:33:1. In some embodiments, the transfer vehicle comprises the helper lipid of DSPC and the PEG-lipid of DMG-PEG(2000), and wherein the molar ratio of ionizable lipid:DSPC:cholesterol:DMG-PEG(2000) is 50:10:38.5:1.5. In some embodiments, the transfer vehicle comprises the helper lipid of DSPC and the PEG-lipid of DSPE-PEG(2000), and wherein the molar ratio of ionizable lipid:DSPC:cholesterol:DSPE-PEG(2000) is 62:4:33:1. In some embodiments, the transfer vehicle comprises the helper lipid of DSPC and the PEG-lipid of DSPE-PEG(2000), and wherein the molar ratio of ionizable lipid:DSPC:cholesterol:DSPE-PEG(2000) is 50:10:38.5:1.5.

In some embodiments, the transfer vehicle comprises the helper lipid of DOPE and the PEG-lipid is C₁₄-PEG(2000), and wherein the molar ratio of ionizable lipid:DOPE:cholesterol:C₁₄-PEG(2000) is 35:16:46.5:2.5. In some embodiments, the transfer vehicle comprises the helper lipid of DSPC and the PEG-lipid is C₁₄-PEG(2000), and wherein the molar ratio of ionizable lipid:DSPC:cholesterol:C₁₄-PEG(2000) is 35:16:46.5:2.5.

In some embodiments, the transfer vehicle comprises the helper lipid of DOPE and the PEG-lipid of DMG-PEG(2000), wherein the molar ratio of ionizable lipid:DOPE:cholesterol:DMG-PEG(2000) is 40:10:40:10. In some embodiments, the transfer vehicle comprises the helper lipid of DSPC and the PEG-lipid of DMG-PEG(2000), wherein the molar ratio of ionizable lipid:DSPC:cholesterol:DMG-PEG(2000) is 40:10:40:10.

In some embodiments, the transfer vehicle has a lipid-nitrogen-to-phosphate (N:P) of about 3 to about 6. In some embodiments, the transfer vehicle has a lipid-nitrogen-to-phosphate (N:P) ratio of about 4, about 4.5, about 5, or about 5.5.

In some embodiments, the transfer vehicle is formulated for endosomal release of the circular RNA polynucleotide.

In some embodiments, the transfer vehicle is capable of binding to APOE. In some embodiments, the transfer vehicle interacts with apolipoprotein E (APOE) less than an equivalent transfer vehicle loaded with a reference linear RNA having the same expression sequence as the circular RNA polynucleotide. In some embodiments, the exterior surface of the transfer vehicle is substantially free of APOE binding sites.

In some embodiments, the transfer vehicle has a diameter of less than about 120 nm. In some embodiments, the transfer vehicle does not form aggregates with a diameter of more than 300 nm.

In some embodiments, the transfer vehicle has an in vivo half-life of less than about 30 hours.

In some embodiments, the transfer vehicle is capable of low density lipoprotein receptor (LDLR) dependent uptake into a cell. In some embodiments, the transfer vehicle is capable of LDLR independent uptake into a cell.

In some embodiments, the pharmaceutical composition is substantially free of linear RNA.

In some embodiments, the pharmaceutical composition further comprises a targeting moiety operably connected to the transfer vehicle. In some embodiments, the targeting moiety specifically binds an immune cell antigen or indirectly. In some embodiments, the immune cell antigen is a T cell antigen. In some embodiments, the T cell antigen is selected from the group consisting of CD2, CD3, CD5, CD7, CD8, CD4, beta7 integrin, beta2 integrin, and C1q.

In some embodiments, the pharmaceutical composition further comprises an adapter molecule comprising a transfer vehicle binding moiety and a cell binding moiety, wherein the targeting moiety specifically binds the transfer vehicle binding moiety and the cell binding moiety specifically binds a target cell antigen. In some embodiments, the target cell antigen is an immune cell antigen. In some embodiments, the immune cell antigen is a T cell antigen, an NK cell, an NKT cell, a macrophage, or a neutrophil. In some embodiments, the T cell antigen is selected from the group consisting of CD2, CD3, CD5, CD7, CD8, CD4, beta7 integrin, beta2 integrin, CD25, CD39, CD73, A2a Receptor, A2b Receptor, and C1q. In some embodiments, the immune cell antigen is a macrophage antigen. In some embodiments, the macrophage antigen is selected from the group consisting of mannose receptor, CD206, and C1q.

In some embodiments, the targeting moiety is a small molecule. In some embodiments, the small molecule binds to an ectoenzyme on an immune cell, wherein the ectoenzyme is selected from the group consisting of CD38, CD73, adenosine 2a receptor, and adenosine 2b receptor. In some embodiments, the small molecule is mannose, a lectin, acivicin, biotin, or digoxigenin.

In some embodiments, the targeting moiety is a single chain Fv (scFv) fragment, nanobody, peptide, peptide-based macrocycle, minibody, small molecule ligand such as folate, arginylglycylaspartic acid (RGD), or phenol-soluble modulin alpha 1 peptide (PSMA1), heavy chain variable region, light chain variable region or fragment thereof.

In some embodiments, the ionizable lipid has a half-life in a cell membrane less than about 2 weeks. In some embodiments, the ionizable lipid has a half-life in a cell membrane less than about 1 week. In some embodiments, the ionizable lipid has a half-life in a cell membrane less than about 30 hours. In some embodiments, the ionizable lipid has a half-life in a cell membrane less than the functional half-life of the circular RNA polynucleotide.

In another aspect, the present application provides a method of treating or preventing a disease, disorder, or condition, comprising administering an effective amount of a pharmaceutical composition disclosed herein. In some embodiments, the disease, disorder, or condition is associated with aberrant expression, activity, or localization of a polypeptide selected from Tables 27 or 28. In some embodiments, the circular RNA polynucleotide encodes a therapeutic protein. In some embodiments, therapeutic protein expression in the spleen is higher than therapeutic protein expression in the liver. In some embodiments, therapeutic protein expression in the spleen is at least about 2.9×therapeutic protein expression in the liver. In some embodiments, the therapeutic protein is not expressed at functional levels in the liver. In some embodiments, the therapeutic protein is not expressed at detectable levels in the liver. In some embodiments, therapeutic protein expression in the spleen is at least about 50% of total therapeutic protein expression. In some embodiments, therapeutic protein expression in the spleen is at least about 63% of total therapeutic protein expression.

In another aspect, the present application provides a linear RNA polynucleotide comprising, from 5′ to 3′, a 3′ group I intron fragment, an Internal Ribosome Entry Site (IRES), an expression sequence, and a 5′ group I intron fragment, further comprising a first spacer 5′ to the 3′ group I intron fragment and/or a second spacer 3′ to the 5′ group I intron fragment.

In some embodiments, the linear RNA polynucleotide comprises a first spacer 5′ to the 3′ group I intron fragment. In some embodiments, the first spacer has a length of 10-50 nucleotides, optionally 10-20 nucleotides, further optionally about 15 nucleotides. In some embodiments, the first spacer comprises a polyA sequence.

In some embodiments, the linear RNA polynucleotide comprises a second spacer 3′ to the 5′ group I intron fragment. In some embodiments, the second spacer has a length of 10-50 nucleotides, optionally 10-20 nucleotides, further optionally about 15 nucleotides. In some embodiments, the second spacer comprises a polyA sequence.

In some embodiments, the linear RNA polynucleotide further comprises a third spacer between the 3′ group I intron fragment and IRES. In some embodiments, the third spacer has a length of about 10 to about 60 nucleotides. In some embodiments, the linear RNA polynucleotide further comprises a first and a second duplex forming regions capable of forming a duplex. In some embodiments, the first and second duplex forming regions each have a length of about 9 to 19 nucleotides. In some embodiments, the first and second duplex forming regions each have a length of about 30 nucleotides.

In some embodiments, the linear RNA polynucleotide has enhanced expression, circularization efficiency, functional stability, and/or stability as compared to a reference linear RNA polynucleotide, wherein the reference linear RNA polynucleotide comprises, from 5′ to 3′, a first polyA sequence, a 5′ external spacer, a 3′ group I intron fragment, a 5′ internal spacer comprising a 5′ internal duplex forming region, an IRES, an expression sequence, a stop condon, a 3′ internal spacer comprising a 3′ internal duplex forming region, a 5′ group I intron fragment, a 3′ external spacer, and a second polyA sequence.

In some embodiments, the linear RNA polynucleotide has enhanced expression, circularization efficiency, functional stability, and/or stability as compared to a reference linear RNA polynucleotide, wherein the reference linear RNA polynucleotide comprises, from 5′ to 3′, a reference 3′ group I intron fragment, a reference IRES, a reference expression sequence, and a reference 5′ group I intron fragment, and does not comprise a spacer 5′ to the 3′ group I intron fragment or a spacer 3′ to the 5′ group I intron fragment. In some embodiments, the expression sequence and the reference expression sequence have the same sequence. In some embodiments, the IRES and the reference IRES have the same sequence.

In some embodiments, the linear RNA polynucleotide comprises a 3′ Anabaena group I intron fragment and a 5′ Anabaena group I intron fragment. In some embodiments, the reference RNA polynucleotide comprises a reference 3′ Anabaena group I intron fragment and a reference 5′ Anabaena group I intron fragment. In some embodiments, the reference 3′ Anabaena group I intron fragment and reference 5′ Anabaena group I intron fragment were generated using the L6-5 permutation site. In some embodiments, the 3′ Anabaena group I intron fragment and 5′ Anabaena group I intron fragment were not generated using the L6-5 permutation site. In some embodiments, the 3′ Anabaena group I intron fragment comprises or consists of a sequence selected from SEQ ID NO: 112-123 and 125-150. In some embodiments, the 5′ Anabaena group I intron fragment comprises a corresponding sequence selected from SEQ ID NO: 73-84 and 86-111. In some embodiments, the 5′ Anabaena group I intron fragment comprises or consists of a sequence selected from SEQ ID NO: 73-84 and 86-111. In some embodiments, the 3′ Anabaena group I intron fragment comprises or consists of a corresponding sequence selected from SEQ ID NO: 112-124 and 125-150.

In some embodiments, the IRES comprises a nucleotide sequence selected from SEQ ID NOs: 348-351. In some embodiments, the reference IRES is CVB3. In some embodiments, the IRES is not CVB3. In some embodiments, the IRES comprises a sequence selected from SEQ ID NOs: 1-64 and 66-72.

In another aspect, the present application discloses a circular RNA polynucleotide produced from the linear RNA disclosed herein.

In another aspect, the present application discloses a circular RNA comprising, from 5′ to 3′, a 3′ group I intron fragment, an IRES, an expression sequence, and a 5′ group I intron fragment, wherein the IRES comprises a nucleotide sequence selected from SEQ ID NOs: 348-351.

In some embodiments, the circular RNA polynucleotide further comprises a spacer between the 3′ group I intron fragment and the IRES.

In some embodiments, the circular RNA polynucleotide further comprises a first and a second duplex forming regions capable of forming a duplex. In some embodiments, the first and second duplex forming regions each have a length of about 9 to 19 nucleotides. In some embodiments, the first and second duplex forming regions each have a length of about 30 nucleotides.

In some embodiments, the expression sequence has a size of at least about 1,000 nt, at least about 2,000 nt, at least about 3,000 nt, at least about 4,000 nt, or at least about 5,000 nt.

In some embodiments, the RNA polynucleotide comprises natural nucleotides. In some embodiments, the expression sequence is codon optimized. In some embodiments, the RNA polynucleotide further comprises a translation termination cassette comprising at least one stop codon in each reading frame. In some embodiments, the translation termination cassette comprises at least two stop codons in the reading frame of the expression sequence. In some embodiments, the RNA polynucleotide is optimized to lack at least one microRNA binding site present in an equivalent pre-optimized polynucleotide. In some embodiments, the RNA polynucleotide is optimized to lack at least one endonuclease susceptible site present in an equivalent pre-optimized polynucleotide. In some embodiments, the RNA polynucleotide is optimized to lack at least one RNA editing susceptible site present in an equivalent pre-optimized polynucleotide.

In some embodiments, the RNA polynucleotide comprises at least 2 expression sequences. In some embodiments, each expression sequence encodes a different therapeutic protein.

In some embodiments, a circular RNA polynucleotide disclosed herein is from about 100 to 15,000 nucleotides, optionally about 100 to 12,000 nucleotides, further optionally about 100 to 10,000 nucleotides in length.

In some embodiments, a circular RNA polynucleotide disclosed herein has an in vivo duration of therapeutic effect in humans of at least about 20 hours. In some embodiments, a circular RNA polynucleotide disclosed herein has a functional half-life of at least about 20 hours. In some embodiments, the circular RNA polynucleotide has a duration of therapeutic effect in a human cell greater than or equal to that of an equivalent linear RNA polynucleotide comprising the same expression sequence. In some embodiments, the circular RNA polynucleotide has a functional half-life in a human cell greater than or equal to that of an equivalent linear RNA polynucleotide comprising the same expression sequence. In some embodiments, the circular RNA polynucleotide has an in vivo duration of therapeutic effect in humans greater than that of an equivalent linear RNA polynucleotide having the same expression sequence. In some embodiments, the circular RNA polynucleotide has an in vivo functional half-life in humans greater than that of an equivalent linear RNA polynucleotide having the same expression sequence.

In another aspect, the present disclosure provides a composition comprising a circular RNA polynucleotide disclosed herein, a nanoparticle, and optionally, a targeting moiety operably connected to the nanoparticle. In some embodiments, the nanoparticle is a lipid nanoparticle, a core-shell nanoparticle, a biodegradable nanoparticle, a biodegradable lipid nanoparticle, a polymer nanoparticle, or a biodegradable polymer nanoparticle. In some embodiments, the pharmaceutical composition comprises a targeting moiety, wherein the targeting moiety mediates receptor-mediated endocytosis or direct fusion selectively into cells of a selected cell population or tissue in the absence of cell isolation or purification. In some embodiments, the targeting moiety is a scfv, nanobody, peptide, minibody, polynucleotide aptamer, heavy chain variable region, light chain variable region or fragment thereof. In some embodiments, wherein less than 1%, by weight, of the polynucleotides in the composition are double stranded RNA, DNA splints, or triphosphorylated RNA. In some embodiments, less than 1%, by weight, of the polynucleotides and proteins in the pharmaceutical composition are double stranded RNA, DNA splints, triphosphorylated RNA, phosphatase proteins, protein ligases, and capping enzymes.

In another aspect, the present disclosure provides a method of treating a subject in need thereof comprising administering a therapeutically effective amount of a composition comprising the circular RNA polynucleotide disclosed herein, a nanoparticle, and optionally, a targeting moiety operably connected to the nanoparticle.

In another aspect, the present disclosure provides a method of treating a subject in need thereof comprising administering a therapeutically effective amount of the pharmaceutical composition disclosed herein. In some embodiments, the targeting moiety is an scfv, nanobody, peptide, minibody, heavy chain variable region, light chain variable region, an extracellular domain of a TCR, or a fragment thereof. In some embodiments, the nanoparticle is a lipid nanoparticle, a core-shell nanoparticle, or a biodegradable nanoparticle. In some embodiments, the nanoparticle comprises one or more cationic lipids, ionizable lipids, or poly β-amino esters. In some embodiments, the nanoparticle comprises one or more non-cationic lipids. In some embodiments, the nanoparticle comprises one or more PEG-modified lipids, polyglutamic acid lipids, or Hyaluronic acid lipids. In some embodiments, the nanoparticle comprises cholesterol. In some embodiments, the nanoparticle comprises arachidonic acid or oleic acid.

In some embodiments, a provided pharmaceutical composition comprises a targeting moiety, wherein the targeting moiety mediates receptor-mediated endocytosis selectively into cells of a selected cell population in the absence of cell selection or purification.

In some embodiments, a provided nanoparticle comprises more than one circular RNA polynucleotide.

In another aspect, the present application provides a DNA vector encoding the RNA polynucleotide disclosed herein. In some embodiments, the DNA vector further comprises a transcription regulatory sequence. In some embodiments, the transcription regulatory sequence comprises a promoter and/or an enhancer. In some embodiments, the promoter comprises a T7 promoter. In some embodiments, the DNA vector comprises a circular DNA. In some embodiments, the DNA vector comprises a linear DNA.

In another aspect, the present application provides a prokaryotic cell comprising the DNA vector disclosed herein.

In another aspect, the present application provides a eukaryotic cell comprising the circular RNA polynucleotide disclosed herein. In some embodiments, the eukaryotic cell is a human cell.

In another aspect, the present application provides a method of producing a circular RNA polynucleotide, the method comprising incubating the linear RNA polynucleotide disclosed herein under suitable conditions for circularization. In some embodiments, the method comprises incubating the DNA disclosed herein under suitable conditions for transcription. In some embodiments, the DNA is transcribed in vitro. In some embodiments, the suitable conditions comprises adenosine triphosphate (ATP), guanine triphosphate (GTP), cytosine triphosphate (CTP), uridine triphosphate (UTP), and an RNA polymerase. In some embodiments, the suitable conditions further comprises guanine monophosphate (GMP). In some embodiments, the ratio of GMP concentration to GTP concentration is within the range of about 3:1 to about 15:1, optionally about 4:1, 5:1, or 6:1.

In another aspect, the present application provides a method of producing a circular RNA polynucleotide, the method comprising culturing the prokaryotic cell disclosed herein under suitable conditions for transcribing the DNA in the cell. In some embodiments, the method further comprising purifying a circular RNA polynucleotide. In some embodiments, the circular RNA polynucleotide is purified by negative selection using an affinity oligonucleotide that hybridizes with the first or second spacer conjugated to a solid surface. In some embodiments, the first or second spacer comprises a polyA sequence, and wherein the affinity oligonucleotide is a deoxythymine oligonucleotide.

In some embodiments of a pharmaceutical composition provided herein, the pharmaceutical composition:liver cell ratio by weight is no more than 1:5. In some embodiments of a pharmaceutical composition provided herein, the pharmaceutical composition:spleen cell ratio by weight is no more than 7:10.

### BRIEF DESCRIPTION OF THE DRAWINGS

FIG. 1 depicts luminescence in supernatants of HEK293 (FIGS. 1A, 1D, and 1E), HepG2 (FIG. 1B), or 1C1C7 (FIG. 1C) cells 24 hours after transfection with circular RNA comprising a Gaussia luciferase expression sequence and various IRES sequences.

FIG. 2 depicts luminescence in supernatants of HEK293 (FIG. 2A), HepG2 (FIG. 2B), or 1C1C7 (FIG. 2C) cells 24 hours after transfection with circular RNA comprising a Gaussia luciferase expression sequence and various IRES sequences having different lengths.

FIG. 3 depicts stability of select IRES constructs in HepG2 (FIG. 3A) or 1C1C7 (FIG. 3B) cells over 3 days as measured by luminescence.

FIGS. 4A and 4B depict protein expression from select IRES constructs in Jurkat cells, as measured by luminescence from secreted Gaussia luciferase in cell supernatants.

FIGS. 5A and 5B depict stability of select IRES constructs in Jurkat cells over 3 days as measured by luminescence.

FIG. 6 depicts comparisons of 24 hour luminescence (FIG. 6A) or relative luminescence over 3 days (FIG. 6B) of modified linear, unpurified circular, or purified circular RNA encoding Gaussia luciferase.

FIG. 7 depicts transcript induction of IFNγ (FIG. 7A), IL-6 (FIG. 7B), IL-2 (FIG. 7C), RIG-I (FIG. 7D), IFN-β1 (FIG. 7E), and TNFα (FIG. 7F) after electroporation of Jurkat cells with modified linear, unpurified circular, or purified circular RNA.

FIG. 8 depicts a comparison of luminescence of circular RNA and modified linear RNA encoding Gaussia luciferase in human primary monocytes (FIG. 8A) and macrophages (FIG. 8B and FIG. 8C).

FIG. 9 depicts relative luminescence over 3 days (FIG. 9A) in supernatant of primary T cells after transduction with circular RNA comprising a Gaussia luciferase expression sequence and varying IRES sequences or 24 hour luminescence (FIG. 9B).

FIG. 10 depicts 24 hour luminescence in supernatant of primary T cells (FIG. 10A) after transduction with circular RNA or modified linear RNA comprising a Gaussia luciferase expression sequence, or relative luminescence over 3 days (FIG. 10B), and 24 hour luminescence in PBMCs (FIG. 10C).

FIG. 11 depicts HPLC chromatograms (FIG. 11A) and circularization efficiencies (FIG. 11B) of RNA constructs having different permutation sites.

FIG. 12 depicts HPLC chromatograms (FIG. 12A) and circularization efficiencies (FIG. 12B) of RNA constructs having different introns and/or permutation sites.

FIG. 13 depicts HPLC chromatograms (FIG. 13A) and circularization efficiencies (FIG. 13B) of 3 RNA constructs with or without homology arms.

FIG. 14 depicts circularization efficiencies of 3 RNA constructs without homology arms or with homology arms having various lengths and GC content.

FIGS. 15A and 15B depict HPLC chromatograms showing the contribution of strong homology arms to improved splicing efficiency, the relationship between circularization efficiency and nicking in select constructs, and combinations of permutations sites and homology arms hypothesized to demonstrate improved circularization efficiency.

FIG. 16 shows fluorescent images of T cells mock electroporated (left) or electroporated with circular RNA encoding a CAR (right) and co-cultured with Raji cells expressing GFP and firefly luciferase.

FIG. 17 shows bright field (left), fluorescent (center), and overlay (right) images of T cells mock electroporated (top) or electroporated with circular RNA encoding a CAR (bottom) and co-cultured with Raji cells expressing GFP and firefly luciferase.

FIG. 18 depicts specific lysis of Raji target cells by T cells mock electroporated or electroporated with circular RNA encoding different CAR sequences.

FIG. 19 depicts luminescence in supernatants of Jurkat cells (left) or resting primary human CD3+ T cells (right) 24 hours after transduction with linear or circular RNA comprising a Gaussia luciferase expression sequence and varying IRES sequences (FIG. 19A), and relative luminescence over 3 days (FIG. 19B).

FIG. 20 depicts transcript induction of IFN-β1 (FIG. 20A), RIG-I (FIG. 20B), IL-2 (FIG. 20C), IL-6 (FIG. 20D), IFNγ (FIG. 20E), and TNFα (FIG. 20F) after electroporation of human CD3+ T cells with modified linear, unpurified circular, or purified circular RNA.

FIG. 21 depicts specific lysis of Raji target cells by human primary CD3+ T cells electroporated with circRNA encoding a CAR as determined by detection of firefly luminescence (FIG. 21A), and IFNγ transcript induction 24 hours after electroporation with different quantities of circular or linear RNA encoding a CAR sequence (FIG. 21B).

FIG. 22 depicts specific lysis of target or non-target cells by human primary CD3+ T cells electroporated with circular or linear RNA encoding a CAR at different E:T ratios (FIG. 22A and FIG. 22B) as determined by detection of firefly luminescence.

FIG. 23 depicts specific lysis of target cells by human CD3+ T cells electroporated with RNA encoding a CAR at 1, 3, 5, and 7 days post electroporation.

FIG. 24 depicts specific lysis of target cells by human CD3+ T cells electroporated with circular RNA encoding a CD19 or BCMA targeted CAR.

FIG. 25 depicts total Flux of organs harvested from CD-1 mice dosed with circular RNA encoding FLuc and formulated with 50% Lipid 15 (Table 10b), 10% DSPC, 1.5% PEG-DMG, and 38.5% cholesterol.

FIG. 26 shows images highlighting the luminescence of organs harvested from CD-1 mice dosed with circular RNA encoding FLuc and formulated with 50% Lipid 15 (Table 10b), 10% DSPC, 1.5% PEG-DMG, and 38.5% cholesterol.

FIG. 27 depicts molecular characterization of Lipids 26 and 27 from Table 10a.

FIG. 27A shows the proton nuclear magnetic resonance (NMR) spectrum of Lipid 26. FIG. 27B shows the retention time of Lipid 26 measured by liquid chromatography-mass spectrometry (LC-MS). FIG. 27C shows the mass spectrum of Lipid 26. FIG. 27D shows the proton NMR spectrum of Lipid 27. FIG. 27E shows the retention time of Lipid 27 measured by LC-MS. FIG. 27F shows the mass spectrum of Lipid 27.

FIG. 28 depicts molecular characterization of Lipid 22-S14 and its synthetic intermediates. FIG. 28A depicts the NMR spectrum of 2-(tetradecylthio)ethan-1-ol. FIG. 28B depicts the NMR spectrum of 2-(tetradecylthio)ethyl acrylate. FIG. 28C depicts the NMR spectrum of bis(2-(tetradecylthio)ethyl) 3,3′-((3-(2-methyl-1H-imidazol-1-yl)propyl)azanediyl)dipropionate (Lipid 22-S14).

FIG. 29 depicts the NMR spectrum of bis(2-(tetradecylthio)ethyl) 3,3′-((3-(1H-imidazol-1-yl)propyl)azanediyl)dipropionate (Lipid 93-S14).

FIG. 30 depicts molecular characterization of heptadecan-9-yl 8-((3-(2-methyl-1H-imidazol-1-yl)propyl)(8-(nonyloxy)-8-oxooctyl)amino)octanoate (Lipid 54 from Table 10a). FIG. 30A shows the proton NMR spectrum of Lipid 54. FIG. 30B shows the retention time of Lipid 54 measured by LC-MS. FIG. 30C shows the mass spectrum of Lipid 54.

FIG. 31 depicts molecular characterization of heptadecan-9-yl 8-((3-(1H-imidazol-1-yl)propyl)(8-(nonyloxy)-8-oxooctyl)amino)octanoate (Lipid 53 from Table 10a). FIG. 31A shows the proton NMR spectrum of Lipid 53. FIG. 31B shows the retention time of Lipid 53 measured by LC-MS. FIG. 31C shows the mass spectrum of Lipid 53.

FIG. 32A depicts total flux of spleen and liver harvested from CD-1 mice dosed with circular RNA encoding firefly luciferase (FLuc) and formulated with ionizable lipid of interest, DSPC, cholesterol, and DSPE-PEG 2000 (Avanti Polar Lipids Inc.) at a weight ratio of 16:1:4:1 or 62:4:33:1 molar ratio. FIG. 32B depicts average radiance for biodistribution of protein expression.

FIG. 33A depicts images highlighting the luminescence of organs harvested from CD-1 mice dosed with circular RNA encoding FLuc and formulated with ionizable Lipid 22-S14, DSPC, cholesterol, and DSPE-PEG 2000 (Avanti Polar Lipids Inc.) at a weight ratio of 16:1:4:1 or 62:4:33:1 molar ratio. FIG. 33B depicts whole body IVIS images of CD-1 mice dosed with circular RNA encoding FLuc and formulated with ionizable Lipid 22-514, DSPC, cholesterol, and DSPE-PEG 2000 (Avanti Polar Lipids Inc.) at a weight ratio of 16:1:4:1 or 62:4:33:1 molar ratio.

FIG. 34A depicts images highlighting the luminescence of organs harvested from CD-1 mice dosed with circular RNA encoding FLuc and formulated with ionizable Lipid 93-S14, DSPC, cholesterol, and DSPE-PEG 2000 (Avanti Polar Lipids Inc.) at a weight ratio of 16:1:4:1 or 62:4:33:1 molar ratio. FIG. 34B depicts whole body IVIS images of CD-1 mice dosed with circular RNA encoding FLuc and formulated with ionizable Lipid 93-S14, DSPC, cholesterol, and DSPE-PEG 2000 (Avanti Polar Lipids Inc.) at a weight ratio of 16:1:4:1 or 62:4:33:1 molar ratio.

FIG. 35A depicts images highlighting the luminescence of organs harvested from CD-1 mice dosed with circular RNA encoding FLuc and formulated with ionizable Lipid 26 from Table 10a, DSPC, cholesterol, and DSPE-PEG 2000 (Avanti Polar Lipids Inc.) at a weight ratio of 16:1:4:1 or 62:4:33:1 molar ratio. FIG. 35B depicts whole body IVIS images of CD-1 mice dosed with circular RNA encoding FLuc and formulated with ionizable Lipid 26, DSPC, cholesterol, and DSPE-PEG 2000 (Avanti Polar Lipids Inc.) at a weight ratio of 16:1:4:1 or 62:4:33:1 molar ratio.

FIG. 36 depicts images highlighting the luminescence of organs harvested from c57BL/6J mice dosed with circular RNA encoding FLuc and encapsulated in lipid nanoparticles formed with Lipid 15 from Table 10b (FIG. 36A), Lipid 53 from Table 10a (FIG. 36B), or Lipid 54 from Table 10a (FIG. 36C). PBS was used as control (FIG. 36D).

FIGS. 37A and 37B depict relative luminescence in the lysates of human PBMCs after 24-hour incubation with testing lipid nanoparticles containing circular RNA encoding firefly luciferase.

FIG. 38 shows the expression of GFP (FIG. 37A) and CD19 CAR (FIG. 37B) in human PBMCs after incubating with testing lipid nanoparticle containing circular RNA encoding either GFP or CD19 CAR.

FIG. 39 depicts the expression of an anti-murine CD19 CAR in 1C1C7 cells lipotransfected with circular RNA comprising an anti-murine CD19 CAR expression sequence and varying IRES sequences.

FIG. 40 shows the cytotoxicity of an anti-murine CD19 CAR to murine T cells. The CD19 CAR is encoded by and expressed from a circular RNA, which is electroporated into the murine T cells.

FIG. 41 depicts the B cell counts in peripheral blood (FIGS. 40A and 40B) or spleen (FIG. 40C) in C57BL/6J mice injected every other day with testing lipid nanoparticles encapsulating a circular RNA encoding an anti-murine CD19 CAR.

FIGS. 42A and 42B compares the expression level of an anti-human CD19 CAR expressed from a circular RNA with that expressed from a linear mRNA.

FIGS. 43A and 43B compares the cytotoxic effect of an anti-human CD19 CAR expressed from a circular RNA with that expressed from a linear mRNA

FIG. 44 depicts the cytotoxicity of two CARs (anti-human CD19 CAR and anti-human BCMA CAR) expressed from a single circular RNA in T cells.

FIG. 45A shows representative FACS plots with frequencies of tdTomato expression in various spleen immune cell subsets following treatment with LNPs formed with Lipid 27 or 26 from Table 10a or Lipid 15 from Table 10b. FIG. 45B shows the quantification of the proportion of myeloid cells, B cells, and T cells expressing tdTomato (mean+std. dev., n=3), equivalent to the proportion of each cell population successfully transfected with Cre circular RNA. FIG. 45C illustrates the proportion of additional splenic immune cell populations, including NK cells, classical monocytes, nonclassical monocytes, neutrophils, and dendritic cells, expressing tdTomato after treatment with Lipids 27 and 26 (mean+std. dev., n=3).

FIG. 46A depicts an exemplary RNA construct design with built-in polyA sequences in the introns. FIG. 46B shows the chromatography trace of unpurified circular RNA. FIG. 46C shows the chromatography trace of affinity-purified circular RNA. FIG. 46D shows the immunogenicity of the circular RNAs prepared with varying IVT conditions and purification methods. (Commercial=commercial IVT mix; Custom=customerized IVT mix; Aff=affinity purification; Enz=enzyme purification; GMP:GTP ratio=8, 12.5, or 13.75).

FIG. 47A depicts an exemplary RNA construct design with a dedicated binding sequence as an alternative to polyA for hybridization purification. FIG. 47B shows the chromatography trace of unpurified circular RNA. FIG. 46C shows the chromatography trace of affinity-purified circular RNA.

FIG. 48A shows the chromatography trace of unpurified circular RNA encoding dystrophin. FIG. 48B shows the chromatography trace of enzyme-purified circular RNA encoding dystrophin.

FIG. 49 compares the expression (FIG. 49A) and stability (FIG. 49B) of purified circRNAs with different 5′ spacers between the 3′ intron fragment/5′ internal duplex region and the IRES in Jurkat cells. (AC=only A and C were used in the spacer sequence; UC=only U and C were used in the spacer sequence.)

FIG. 50 shows luminescence expression levels and stability of expression in primary T cells from circular RNAs containing the original or modified IRES elements indicated.

FIG. 51 shows luminescence expression levels and stability of expression in HepG2 cells from circular RNAs containing the original or modified IRES elements indicated.

FIG. 52 shows luminescence expression levels and stability of expression in 1C1C7 cells from circular RNAs containing the original or modified IRES elements indicated.

FIG. 53 shows luminescence expression levels and stability of expression in HepG2 cells from circular RNAs containing IRES elements with untranslated regions (UTRs) inserted or hybrid IRES elements. “Scr” means Scrambled, which was used as a control.

FIG. 54 shows luminescence expression levels and stability of expression in 1C1C7 cells from circular RNAs containing an IRES and variable stop codon cassettes operably linked to a Gaussia luciferase coding sequence.

FIG. 55 shows luminescence expression levels and stability of expression in 1C1C7 cells from circular RNAs containing an IRES and variable untranslated regions (UTRs) inserted before the start codon of a gaussian luciferase coding sequence.

FIG. 56 shows expression levels of human erythropoietin (hEPO) in Huh7 cells from circular RNAs containing two miR-122 target sites downstream from the hEPO coding sequence.

FIG. 57 shows luminescence expression levels in SupT1 cells (from a human T cell tumor line) and MV4-11 cells (from a human macrophage line) from LNPs transfected with circular RNAs encoding for Firefly luciferase in vitro.

FIG. 58 shows a comparison of transfected primary human T cells LNPs containing circular RNAs dependency of ApoE based on the different helper lipid, PEG lipid, and ionizable lipid:phosphate ratio formulations.

FIG. 59 shows uptake of LNP containing circular RNAs encoding eGFP into activated primary human T cells with or without the aid of ApoE3.

FIG. 60 shows immune cell expression from a LNP containing circular RNA encoding for a Cre fluorescent protein in a Cre reporter mouse model.

FIG. 61 shows immune cell expression of mOX40L in wildtype mice following intravenous injection of LNPs that have been transfected with circular RNAs encoding mOX40L.

FIG. 62 shows single dose of mOX40L in LNPs transfected with circular RNAs capable of expressing mOX40L. FIGS. 62A and 62B provide percent of mOX40L expression in splenic T cells, CD4+ T cells, CD8+ T cells, B cells, NK cells, dendritic cells, and other myloid cells. FIG. 62C provides mouse weight change 24 hours after transfection.

FIG. 63 shows B cell depletion of LNPs transfected intravenously with circular RNAs in mice. FIG. 63A quantifies Be cell depletion through B220+ B cells of live, CD45+ immune cells and FIG. 63B compares B cell depletion of B220+ B cells of live, CD45+ immune cells in comparison to luciferase expressing circular RNAs. FIG. 63C provides B cell weight gain of the transfected cells.

FIG. 64 shows CAR expression levels in the peripheral blood (FIG. 64A) and spleen (FIG. 64B) when treated with LNP encapsulating circular RNA that expresses anti-CD19 CAR. Anti-CD20 (aCD20) and circular RNA encoding luciferase (oLuc) were used for comparison.

FIG. 65 shows the overall frequency of anti-CD19 CAR expression, the frequency of anti-CD19 CAR expression on the surface of cells and effect on anti-tumor response of IRES specific circular RNA encoding anti-CD19 CARs on T-cells. FIG. 65A shows anti-CD19 CAR geometric mean florescence intensity, FIG. 65B shows percentage of anti-CD19 CAR expression, and FIG. 65C shows the percentage target cell lysis performed by the anti-CD19 CAR. (CK=Caprine Kobuvirus; AP=Apodemus Picornavirus; CK*=Caprine Kobuvirus with codon optimization; PV=Parabovirus; SV=Salivirus.)

FIG. 66 shows CAR expression levels of A20 FLuc target cells when treated with IRES specific circular RNA constructs.

FIG. 67 shows luminescence expression levels for cytosolic (FIG. 67A) and surface (FIG. 67B) proteins from circular RNA in primary human T-cells.

FIG. 68 shows luminescence expression in human T-cells when treated with IRES specific circular constructs. Expression in circular RNA constructs were compared to linear mRNA. FIG. 68A, FIG. 68B, and FIG. 68G provide Gaussia luciferase expression in multiple donor cells. FIG. 68C, FIG. 68D, FIG. 68E, and FIG. 68F provides firefly luciferase expression in multiple donor cells.

FIG. 69 shows anti-CD19 CAR (FIG. 69A and FIG. 69B) and anti-BCMA CAR (FIG. 68B) expression in human T-cells following treatment of a lipid nanoparticle encompassing a circular RNA that encodes either an anti-CD19 or anti-BCMA CAR to a firefly luciferase expressing K562 cell.

FIG. 70 shows anti-CD19 CAR expression levels resulting from delivery via electroporation in vitro of a circular RNA encoding an anti-CD19 CAR in a specific antigen-dependent manner. FIG. 70A shows Nalm6 cell lysing with an anti-CD19 CAR. FIG. 70B shows K562 cell lysing with an anti-CD19 CAR.

FIG. 71 shows transfection of LNP mediated by use of ApoE3 in solutions containing LNP and circular RNA expressing green fluorescence protein (GFP). FIG. 71A showed the live-dead results. FIG. 71B, FIG. 71C, FIG. 71D, and FIG. 71E provide the frequency of expression for multiple donors.

FIG. 72 shows total flux and percent expression for varying lipid formulations from Table 10a.

### DETAILED DESCRIPTION

Provided herein are pharmaceutical compositions and transfer vehicles, e.g., lipid nanoparticles, comprising circular RNA. The circular RNA provided herein may be delivered and/or targeted to a cell in a transfer vehicle, e.g., a nanoparticle, or a composition comprising a transfer vehicle. In some embodiments, the circular RNA may also be delivered to a subject in a transfer vehicle or a composition comprising a transfer vehicle. In some embodiments, the transfer vehicle is a nanoparticle. In some embodiments, the nanoparticle is a lipid nanoparticle, a polymeric core-shell nanoparticle, or a biodegradable nanoparticle. In some embodiments, the nanoparticle is a lipid nanoparticle. In some embodiments, the transfer vehicle comprises one or more ionizable lipids, PEG modified lipids, helper lipids, and/or structural lipids.

In some embodiments, a transfer vehicle encapsulates circular RNA and comprises an ionizable lipid, a structural lipid, and a PEG-modified lipid. In some embodiments, a transfer vehicle encapsulates circular RNA and comprises an ionizable lipid, a structural lipid, a PEG-modified lipid, and a helper lipid.

In some embodiments, the transfer vehicle comprises an ionizable lipid described herein. In some embodiments, the transfer vehicle comprises an ionizable lipid shown in any one of Tables 1-10, 10a, 10b, 11-15, and 15b. In some embodiments, the transfer vehicle comprises an ionizable lipid shown in Table 10a.

In some embodiments, the RNA in a transfer vehicle is at least about 80%, 85%, 90%, 91%, 92%, 93%, 94%, 95%, 96%, 97%, 98%, 99%, 99.5%, 99.9%, or more circular RNA. In some embodiments, less than 5%, 10%, 15%, 20%, 25%, 30%, 35%, 40%, 45%, 50%, 55%, 60%, 65%, or 70% of loaded RNA is on or associated with a transfer vehicle exterior surface.

In some embodiments, the transfer vehicle is capable of binding to APOE. In some embodiments, the surface of the transfer vehicle comprises APOE binding sites. In some embodiments, the surface of the transfer vehicle is substantially free of APOE binding sites. In some embodiments, a transfer vehicle interacts with APOE less than an equivalent transfer vehicle loaded with linear RNA. In some embodiments, APOE interaction may be measured by comparing nanoparticle uptake in cells in APO depleted serum or APO complement serum.

Without wishing to be bound by theory, it is contemplated that transfer vehicles comprising APOE binding sites deliver circular RNAs more efficiently to the liver. Accordingly, in some embodiments, the transfer vehicle comprising the ionizable lipids described herein and loaded with circular RNA substantially comprises APOE binding sites on the transfer vehicle surface, thereby delivering the circular RNA to the liver at a higher efficiency compared to a transfer vehicle substantially lacking APOE binding sites on the surface. In some embodiments, the transfer vehicle comprising the ionizable lipids described herein and loaded with circular RNA substantially lacks APOE binding sites on the transfer vehicle surface, thereby delivering the circular RNA to the liver at a lower efficiency compared to a transfer vehicle comprising APOE binding sites on the surface.

In some embodiments, the transfer vehicle delivers, or is capable of delivering, circular RNA to the spleen. In some embodiments, a circular RNA encodes a therapeutic protein. In some embodiments, at least about 10%, 15%, 20%, 25%, 30%, 35%, 40%, 45%, 50%, 55%, 60%, 65%, 70%, 75%, 80%, 85%, 90%, 95%, or 99% of the total therapeutic protein expressed in the subject is expressed in the spleen. In some embodiments, more therapeutic protein is expressed in the spleen than in the liver (e.g., 2×, 3×, 4×, or 5× more). In some embodiments, the lipid nanoparticle has an ionizable lipid:phosphate ratio of 3-7. In some embodiments, the lipid nanoparticle has an ionizable lipid:phosphate ratio of 4-6. In some embodiments, the lipid nanoparticle has an ionizable lipid:phosphate ratio of 4.5. In some embodiments, the lipid nanoparticle has an nitrogen:phosphate (N:P) ratio of 3-6. In some embodiments, the lipid nanoparticle has an N:P ratio of 5-6. In some embodiments, the lipid nanoparticle has an N:P ratio of 5.7. In some embodiments, expression of a nonsecreted protein may be measured using an ELISA, normalizing to tissue weight.

Without wishing to be bound by theory, it is thought that transfer vehicles described herein shield encapsulated circular RNA from degradation and provide for effective delivery of circular RNA to target cells in vivo and in vitro.

Embodiments of the present disclosure provide lipid compositions described according to the respective molar ratios of the component lipids in the formulation. In one embodiment, the mol-% of the ionizable lipid may be from about 10 mol-% to about 80 mol-%. In one embodiment, the mol-% of the ionizable lipid may be from about 20 mol-% to about 70 mol-%. In one embodiment, the mol-% of the ionizable lipid may be from about 30 mol-% to about 60 mol-%. In one embodiment, the mol-% of the ionizable lipid may be from about 35 mol-% to about 55 mol-%. In one embodiment, the mol-% of the ionizable lipid may be from about 40 mol-% to about 50 mol-%. In some embodiments, the ionizable lipid mol-% of the transfer vehicle batch will be ±30%, ±25%, ±20%, ±15%, ±10%, ±5%, or ±2.5% of the target mol-%. In certain embodiments, transfer vehicle inter-lot variability will be less than 15%, less than 10% or less than 5%.

In one embodiment, the mol-% of the helper lipid may be from about 1 mol-% to about 50 mol-%. In one embodiment, the mol-% of the helper lipid may be from about 2 mol-% to about 45 mol-%. In one embodiment, the mol-% of the helper lipid may be from about 3 mol-% to about 40 mol-%. In one embodiment, the mol-% of the helper lipid may be from about 4 mol-% to about 35 mol-%. In one embodiment, the mol-% of the helper lipid may be from about 5 mol-% to about 30 mol-%. In one embodiment, the mol-% of the helper lipid may be from about 10 mol-% to about 20 mol-%. In some embodiments, the helper lipid mol-% of the transfer vehicle batch will be ±30%, ±25%, ±20%, ±15%, ±10%, ±5%, or ±2.5% of the target mol-%.

In one embodiment, the mol-% of the structural lipid may be from about 10 mol-% to about 80 mol-%. In one embodiment, the mol-% of the structural lipid may be from about 20 mol-% to about 70 mol-%. In one embodiment, the mol-% of the structural lipid may be from about 30 mol-% to about 60 mol-%. In one embodiment, the mol-% of the structural lipid may be from about 35 mol-% to about 55 mol-%. In one embodiment, the mol-% of the structural lipid may be from about 40 mol-% to about 50 mol-%. In some embodiments, the structural lipid mol-% of the transfer vehicle batch will be ±30%, ±25%, ±20%, ±15%, ±10%, ±5%, or ±2.5% of the target mol-%.

In one embodiment, the mol-% of the PEG modified lipid may be from about 0.1 mol-% to about 10 mol-%. In one embodiment, the mol-% of the PEG modified lipid may be from about 0.2 mol-% to about 5 mol-%. In one embodiment, the mol-% of the PEG modified lipid may be from about 0.5 mol-% to about 3 mol-%. In one embodiment, the mol-% of the PEG modified lipid may be from about 1 mol-% to about 2 mol-%. In one embodiment, the mol-% of the PEG modified lipid may be about 1.5 mol-%. In some embodiments, the PEG modified lipid mol-% of the transfer vehicle batch will be ±30%, ±25%, ±20%, ±15%, ±10%, ±5%, or ±2.5% of the target mol-%.

Also contemplated are pharmaceutical compositions, and in particular transfer vehicles, that comprise one or more of the compounds disclosed herein. In certain embodiments, such transfer vehicles comprise one or more of PEG-modified lipids, an ionizable lipid, a helper lipid, and/or a structural lipid disclosed herein. Also contemplated are transfer vehicles that comprise one or more of the compounds disclosed herein and that further comprise one or more additional lipids. In certain embodiments, such transfer vehicles are loaded with or otherwise encapsulate circular RNA.

Transfer vehicles of the invention encapsulate circular RNA. In certain embodiments, the polynucleotides encapsulated by the compounds or pharmaceutical and liposomal compositions of the invention include RNA encoding a protein or enzyme (e.g., circRNA encoding, for example, phenylalanine hydroxylase (PAH)). The present invention contemplates the use of such polynucleotides as a therapeutic that is capable of being expressed by target cells to thereby facilitate the production (and in certain instances, the excretion) of a functional enzyme or protein as disclosed by such target cells, for example, in International Application No. PCT/US2010/058457 and in U.S. Provisional Application No. 61/494,881, filed Jun. 8, 2011, the teachings of which are both incorporated herein by reference in their entirety. For example, in certain embodiments, upon the expression of one or more polynucleotides by target cells, the production of a functional enzyme or protein in which a subject is deficient (e.g., a urea cycle enzyme or an enzyme associated with a lysosomal storage disorder) may be observed. As another example, circular RNA encapsulated by a transfer vehicle may encode one or both polypeptide chains of a T cell receptor protein or encode a chimeric antigen receptor (CAR).

Also provided herein are methods of treating a disease in a subject by administering an effective amount of a composition comprising circular RNA encoding a functional protein and a transfer vehicle described herein to the subject. In some embodiments, the circular RNA is encapsulated within the transfer vehicle. In certain embodiments, such methods may enhance (e.g., increase) the expression of a polynucleotide and/or increase the production and secretion of a functional polypeptide product in one or more target cells and tissues (e.g., immune cells or hepatocytes). Generally, such methods comprise contacting the target cells with one or more compounds and/or transfer vehicles that comprise or otherwise encapsulate the circRNA.

In certain embodiments, the transfer vehicles (e.g., lipid nanoparticles) are formulated based in part upon their ability to facilitate the transfection (e.g., of a circular RNA) of a target cell. In another embodiment, the transfer vehicles (e.g., lipid nanoparticles) may be selected and/or prepared to optimize delivery of circular RNA to a target cell, tissue or organ. For example, if the target cell is a hepatocyte, or if the target organ is the spleen, the properties of the pharmaceutical and/or liposomal compositions (e.g., size, charge and/or pH) may be optimized to effectively deliver such composition (e.g., lipid nanoparticles) to the target cell or organ, reduce immune clearance and/or promote retention in the target cell or organ. Alternatively, if the target tissue is the central nervous system, the selection and preparation of the transfer vehicle must consider penetration of, and retention within. the blood brain barrier and/or the use of alternate means of directly delivering such compositions (e.g., lipid nanoparticles) to such target tissue (e.g., via intracerebrovascular administration). In certain embodiments, the transfer vehicles may be combined with agents that facilitate the transfer of encapsulated materials across the blood brain barrier (e.g., agents which disrupt or improve the permeability of the blood brain barrier and thereby enhance the transfer of circular RNA to the target cells). While the transfer vehicles described herein (e.g., lipid nanoparticles) can facilitate introduction of circRNA into target cells, the addition of polycations (e.g., poly L-lysine and protamine) to, for example, one or more of the lipid nanoparticles that comprise the pharmaceutical compositions as a copolymer can also facilitate, and in some instances markedly enhance, the transfection efficiency of several types of transfer vehicles by 2-28 fold in a number of cell lines both in vitro and in vivo (See, N. J. Caplen, et al., Gene Ther. 1995; 2: 603; S. Li, et al., Gene Ther. 1997; 4, 891.). In some embodiments, a target cell is an immune cell. In some embodiments, a target cell is a T cell.

In certain embodiments, the transfer vehicles described herein (e.g., lipid nanoparticles) are prepared by combining multiple lipid components (e.g., one or more of the compounds disclosed herein) with one or more polymer components. For example, a lipid nanoparticle may be prepared using HGT4003, DOPE, cholesterol and DMG-PEG2000. A lipid nanoparticle may be comprised of additional lipid combinations in various ratios, including for example, HGT4001, DOPE and DMG-PEG2000. The selection of ionizable lipids, helper lipids, structural lipids, and/or PEG-modified lipids which comprise the lipid nanoparticles, as well as the relative molar ratio of such lipids to each other, is based upon the characteristics of the selected lipid(s), the nature of the intended target cells or tissues and the characteristics of the materials or polynucleotides to be delivered by the lipid nanoparticle. Additional considerations include, for example, the saturation of the alkyl chain, as well as the size, charge, pH, pKa, fusogenicity and toxicity of the selected lipid(s).

Transfer vehicles described herein can allow the encapsulated polynucleotide to reach the target cell or may preferentially allow the encapsulated polynucleotide to reach the target cells or organs on a discriminatory basis (e.g., the transfer vehicles may concentrate in the liver or spleen of a subject to which such transfer vehicles are administered). Alternatively, the transfer vehicles may limit the delivery of encapsulated polynucleotides to other non-targeted cells or organs where the presence of the encapsulated polynucleotides may be undesirable or of limited utility.

Loading or encapsulating a polynucleotide, e.g., circRNA, into a transfer vehicle may serve to protect the polynucleotide from an environment (e.g., serum) which may contain enzymes or chemicals that degrade such polynucleotides and/or systems or receptors that cause the rapid excretion of such polynucleotides. Accordingly, in some embodiments, the compositions described herein are capable of enhancing the stability of the encapsulated polynucleotide(s), particularly with respect to the environments into which such polynucleotides will be exposed.

In certain embodiments, provided herein is a vector for making circular RNA, the vector comprising a 5′ duplex forming region, a 3′ group I intron fragment, optionally a first spacer, an Internal Ribosome Entry Site (IRES), an expression sequence, optionally a second spacer, a 5′ group I intron fragment, and a 3′ duplex forming region. In some embodiments, these elements are positioned in the vector in the above order. In some embodiments, the vector further comprises an internal 5′ duplex forming region between the 3′ group I intron fragment and the IRES and an internal 3′ duplex forming region between the expression sequence and the 5′ group I intron fragment. In some embodiments, the internal duplex forming regions are capable of forming a duplex between each other but not with the external duplex forming regions. In some embodiments, the internal duplex forming regions are part of the first and second spacers. Additional embodiments include circular RNA polynucleotides, including circular RNA polynucleotides made using the vectors provided herein, compositions comprising such circular RNA, cells comprising such circular RNA, methods of using and making such vectors, circular RNA, compositions and cells.

In some embodiments, provided herein are methods comprising administration of circular RNA polynucleotides provided herein into cells for therapy or production of useful proteins, such as PAH. In some embodiments, the method is advantageous in providing the production of a desired polypeptide inside eukaryotic cells with a longer half-life than linear RNA, due to the resistance of the circular RNA to ribonucleases.

Circular RNA polynucleotides lack the free ends necessary for exonuclease-mediated degradation, causing them to be resistant to several mechanisms of RNA degradation and granting extended half-lives when compared to an equivalent linear RNA. Circularization may allow for the stabilization of RNA polynucleotides that generally suffer from short half-lives and may improve the overall efficacy of exogenous mRNA in a variety of applications. In an embodiment, the half-life of the circular RNA polynucleotides provided herein in eukaryotic cells (e.g., mammalian cells, such as human cells) is at least 20 hours (e.g., at least 80 hours).

### 1. Definitions

As used herein, the terms “circRNA” or “circular polyribonucleotide” or “circular RNA” or “oRNA” are used interchangeably and refers to a polyribonucleotide that forms a circular structure through covalent bonds.

As used herein, the term “3′ group I intron fragment” refers to a sequence with 75% or higher similarity to the 3′-proximal end of a natural group I intron including the splice site dinucleotide and optionally a stretch of natural exon sequence.

As used herein, the term “5′ group I intron fragment” refers to a sequence with 75% or higher similarity to the 5′-proximal end of a natural group I intron including the splice site dinucleotide and optionally a stretch of natural exon sequence.

As used herein, the term “permutation site” refers to the site in a group I intron where a cut is made prior to permutation of the intron. This cut generates 3′ and 5′ group I intron fragments that are permuted to be on either side of a stretch of precursor RNA to be circularized.

As used herein, the term “splice site” refers to a dinucleotide that is partially or fully included in a group I intron and between which a phosphodiester bond is cleaved during RNA circularization.

As used herein, the term “therapeutic protein” refers to any protein that, when administered to a subject directly or indirectly in the form of a translated nucleic acid, has a therapeutic, diagnostic, and/or prophylactic effect and/or elicits a desired biological and/or pharmacological effect.

As used herein, the term “immunogenic” refers to a potential to induce an immune response to a substance. An immune response may be induced when an immune system of an organism or a certain type of immune cells is exposed to an immunogenic substance. The term “non-immunogenic” refers to a lack of or absence of an immune response above a detectable threshold to a substance. No immune response is detected when an immune system of an organism or a certain type of immune cells is exposed to a non-immunogenic substance. In some embodiments, a non-immunogenic circular polyribonucleotide as provided herein, does not induce an immune response above a pre-determined threshold when measured by an immunogenicity assay. In some embodiments, no innate immune response is detected when an immune system of an organism or a certain type of immune cells is exposed to a non-immunogenic circular polyribonucleotide as provided herein. In some embodiments, no adaptive immune response is detected when an immune system of an organism or a certain type of immune cell is exposed to a non-immunogenic circular polyribonucleotide as provided herein.

As used herein, the term “circularization efficiency” refers to a measurement of resultant circular polyribonucleotide as compared to its linear starting material.

As used herein, the term “translation efficiency” refers to a rate or amount of protein or peptide production from a ribonucleotide transcript. In some embodiments, translation efficiency can be expressed as amount of protein or peptide produced per given amount of transcript that codes for the protein or peptide.

The term “nucleotide” refers to a ribonucleotide, a deoxyribonucleotide, a modified form thereof, or an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Nucleotide analogs include nucleotides having modifications in the chemical structure of the base, sugar and/or phosphate, including, but not limited to, 5′-position pyrimidine modifications, 8′-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety as defined herein. Nucleotide analogs are also meant to include nucleotides with bases such as inosine, queuosine, xanthine; sugars such as 2′-methyl ribose; non-natural phosphodiester linkages such as methylphosphonate, phosphorothioate and peptide linkages. Nucleotide analogs include 5-methoxyuridine, 1-methylpseudouridine, and 6-methyladenosine.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, or up to about 10,000 or more bases, composed of nucleotides, e.g., deoxyribonucleotides or ribonucleotides, and may be produced enzymatically or synthetically (e.g., as described in U.S. Pat. No. 5,948,902 and the references cited therein), which can hybridize with naturally occurring nucleic acids in a sequence specific manner analogous to that of two naturally occurring nucleic acids, e.g., can participate in Watson-Crick base pairing interactions. Naturally occurring nucleic acids are comprised of nucleotides including guanine, cytosine, adenine, thymine, and uracil (G, C, A, T, and U respectively).

The terms “ribonucleic acid” and “RNA” as used herein mean a polymer composed of ribonucleotides.

The terms “deoxyribonucleic acid” and “DNA” as used herein mean a polymer composed of deoxyribonucleotides.

“Isolated” or “purified” generally refers to isolation of a substance (for example, in some embodiments, a compound, a polynucleotide, a protein, a polypeptide, a polynucleotide composition, or a polypeptide composition) such that the substance comprises a significant percent (e.g., greater than 1%, greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90%-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is more than as it is found naturally.

The terms “duplexed,” “double-stranded,” or “hybridized” as used herein refer to nucleic acids formed by hybridization of two single strands of nucleic acids containing complementary sequences. In most cases, genomic DNA is double-stranded. Sequences can be fully complementary or partially complementary.

As used herein, “unstructured” with regard to RNA refers to an RNA sequence that is not predicted by the RNAFold software or similar predictive tools to form a structure (e.g., a hairpin loop) with itself or other sequences in the same RNA molecule. In some embodiments, unstructured RNA can be functionally characterized using nuclease protection assays.

As used herein, “structured” with regard to RNA refers to an RNA sequence that is predicted by the RNAFold software or similar predictive tools to form a structure (e.g., a hairpin loop) with itself or other sequences in the same RNA molecule.

As used herein, two “duplex forming regions,” “homology arms,” or “homology regions,” may be any two regions that are thermodynamically favored to cross-pair in a sequence specific interaction. In some embodiments, two duplex forming regions, homology arms, or homology regions, share a sufficient level of sequence identity to one another's reverse complement to act as substrates for a hybridization reaction. As used herein polynucleotide sequences have “homology” when they are either identical or share sequence identity to a reverse complement or “complementary” sequence. The percent sequence identity between a homology region and a counterpart homology region's reverse complement can be any percent of sequence identity that allows for hybridization to occur. In some embodiments, an internal duplex forming region of an inventive polynucleotide is capable of forming a duplex with another internal duplex forming region and does not form a duplex with an external duplex forming region.

Linear nucleic acid molecules are said to have a “5′-terminus” (5′ end) and a “3′-terminus” (3′ end) because nucleic acid phosphodiester linkages occur at the 5′ carbon and 3′ carbon of the sugar moieties of the substituent mononucleotides. The end nucleotide of a polynucleotide at which a new linkage would be to a 5′ carbon is its 5′ terminal nucleotide. The end nucleotide of a polynucleotide at which a new linkage would be to a 3′ carbon is its 3′ terminal nucleotide. A terminal nucleotide, as used herein, is the nucleotide at the end position of the 3′- or 5′-terminus

“Transcription” means the formation or synthesis of an RNA molecule by an RNA polymerase using a DNA molecule as a template. The invention is not limited with respect to the RNA polymerase that is used for transcription. For example, in some embodiments, a T7-type RNA polymerase can be used.

“Translation” means the formation of a polypeptide molecule by a ribosome based upon an RNA template.

It is to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. Thus, for example, reference to “a cell” includes combinations of two or more cells, or entire cultures of cells; reference to “a polynucleotide” includes, as a practical matter, many copies of that polynucleotide. Unless specifically stated or obvious from context, as used herein, the term “or” is understood to be inclusive. Unless defined herein and below in the reminder of the specification, all technical and scientific terms used herein have the same meaning as commonly understood by one of ordinary skill in the art to which the invention pertains.

Unless specifically stated or obvious from context, as used herein, the term “about,” is understood as within a range of normal tolerance in the art, for example within 2 standard deviations of the mean. “About” can be understood as within 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01% of the stated value. Unless otherwise clear from the context, all numerical values provided herein are modified by the term “about.”

As used herein, the term “encode” refers broadly to any process whereby the information in a polymeric macromolecule is used to direct the production of a second molecule that is different from the first. The second molecule may have a chemical structure that is different from the chemical nature of the first molecule.

By “co-administering” is meant administering a therapeutic agent provided herein in conjunction with one or more additional therapeutic agents sufficiently close in time such that the therapeutic agent provided herein can enhance the effect of the one or more additional therapeutic agents, or vice versa.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. The treatment or prevention provided by the method disclosed herein can include treatment or prevention of one or more conditions or symptoms of the disease. Also, for purposes herein, “prevention” can encompass delaying the onset of the disease, or a symptom or condition thereof.

As used herein, the term “expression sequence” refers to a nucleic acid sequence that encodes a product, e.g., a peptide or polypeptide, regulatory nucleic acid, or non-coding nucleic acid. An exemplary expression sequence that codes for a peptide or polypeptide can comprise a plurality of nucleotide triads, each of which can code for an amino acid and is termed as a “codon”.

As used herein, a “spacer” refers to a region of a polynucleotide sequence ranging from 1 nucleotide to hundreds or thousands of nucleotides separating two other elements along a polynucleotide sequence. The sequences can be defined or can be random. A spacer is typically non-coding. In some embodiments, spacers include duplex forming regions.

As used herein, “splice site” refers to the dinucleotide or dinucleotides between which cleavage of the phosphodiester bond occurs during a splicing reaction. A “5′ splice site” refers to the natural 5′ dinucleotide of the intron e.g., group I intron, while a “3′ splice site” refers to the natural 3′ dinucleotide of the intron.

As used herein, an “internal ribosome entry site” or “IRES” refers to an RNA sequence or structural element ranging in size from 10 nt to 1000 nt or more, capable of initiating translation of a polypeptide in the absence of a typical RNA cap structure. An IRES is typically about 500 nt to about 700 nt in length.

As used herein, a “miRNA site” refers to a stretch of nucleotides within a polynucleotide that is capable of forming a duplex with at least 8 nucleotides of a natural miRNA sequence.

As used herein, an “endonuclease site” refers to a stretch of nucleotides within a polynucleotide that is capable of being recognized and cleaved by an endonuclease protein.

As used herein, “bicistronic RNA” refers to a polynucleotide that includes two expression sequences coding for two distinct proteins. These expression sequences can be separated by a nucleotide sequence encoding a cleavable peptide such as a protease cleavage site. They can also be separated by a ribosomal skipping element.

As used herein, the term“ribosomal skipping element” refers to a nucleotide sequence encoding a short peptide sequence capable of causing generation of two peptide chains from translation of one RNA molecule. While not wishing to be bound by theory, it is hypothesized that ribosomal skipping elements function by (1) terminating translation of the first peptide chain and re-initiating translation of the second peptide chain; or (2) cleavage of a peptide bond in the peptide sequence encoded by the ribosomal skipping element by an intrinsic protease activity of the encoded peptide, or by another protease in the environment (e.g., cytosol).

As used herein, the term “co-formulate” refers to a nanoparticle formulation comprising two or more nucleic acids or a nucleic acid and other active drug substance. Typically, the ratios are equimolar or defined in the ratiometric amount of the two or more nucleic acids or the nucleic acid and other active drug substance.

As used herein, “transfer vehicle” includes any of the standard pharmaceutical carriers, diluents, excipients, and the like, which are generally intended for use in connection with the administration of biologically active agents, including nucleic acids.

As used herein, the phrase “lipid nanoparticle” refers to a transfer vehicle comprising one or more lipids (e.g., in some embodiments, cationic lipids, non-cationic lipids, and PEG-modified lipids).

As used herein, the phrase “ionizable lipid” refers to any of a number of lipid species that carry a net positive charge at a selected pH, such as physiological pH 4 and a neutral charge at other pHs such as physiological pH 7.

In some embodiments, a lipid, e.g., an ionizable lipid, disclosed herein comprises one or more cleavable groups. The terms “cleave” and “cleavable” are used herein to mean that one or more chemical bonds (e.g., one or more of covalent bonds, hydrogen-bonds, van der Waals' forces and/or ionic interactions) between atoms in or adjacent to the subject functional group are broken (e.g., hydrolyzed) or are capable of being broken upon exposure to selected conditions (e.g., upon exposure to enzymatic conditions). In certain embodiments, the cleavable group is a disulfide functional group, and in particular embodiments is a disulfide group that is capable of being cleaved upon exposure to selected biological conditions (e.g., intracellular conditions). In certain embodiments, the cleavable group is an ester functional group that is capable of being cleaved upon exposure to selected biological conditions. For example, the disulfide groups may be cleaved enzymatically or by a hydrolysis, oxidation or reduction reaction. Upon cleavage of such disulfide functional group, the one or more functional moieties or groups (e.g., one or more of a head-group and/or a tail-group) that are bound thereto may be liberated. Exemplary cleavable groups may include, but are not limited to, disulfide groups, ester groups, ether groups, and any derivatives thereof (e.g., alkyl and aryl esters). In certain embodiments, the cleavable group is not an ester group or an ether group. In some embodiments, a cleavable group is bound (e.g., bound by one or more of hydrogen-bonds, van der Waals' forces, ionic interactions and covalent bonds) to one or more functional moieties or groups (e.g., at least one head-group and at least one tail-group). In certain embodiments, at least one of the functional moieties or groups is hydrophilic (e.g., a hydrophilic head-group comprising one or more of imidazole, guanidinium, amino, imine, enamine, optionally-substituted alkyl amino and pyridyl).

As used herein, the term “hydrophilic” is used to indicate in qualitative terms that a functional group is water-preferring, and typically such groups are water-soluble. For example, disclosed herein are compounds that comprise a cleavable disulfide (S—S) functional group bound to one or more hydrophilic groups (e.g., a hydrophilic head-group), wherein such hydrophilic groups comprise or are selected from the group consisting of imidazole, guanidinium, amino, imine, enamine, an optionally-substituted alkyl amino (e.g., an alkyl amino such as dimethylamino) and pyridyl.

In certain embodiments, at least one of the functional groups of moieties that comprise the compounds disclosed herein is hydrophobic in nature (e.g., a hydrophobic tail-group comprising a naturally occurring lipid such as cholesterol). As used herein, the term “hydrophobic” is used to indicate in qualitative terms that a functional group is water-avoiding, and typically such groups are not water soluble. For example, disclosed herein are compounds that comprise a cleavable functional group (e.g., a disulfide (S—S) group) bound to one or more hydrophobic groups, wherein such hydrophobic groups comprise one or more naturally occurring lipids such as cholesterol, and/or an optionally substituted, variably saturated or unsaturated C₆-C₂₀ alkyl and/or an optionally substituted, variably saturated or unsaturated C₆-C₂₀ acyl.

Compound described herein may also comprise one or more isotopic substitutions. For example, H may be in any isotopic form, including ¹H, ²H (D or deuterium), and ³H (T or tritium); C may be in any isotopic form, including ¹³C, and ¹⁴C; O may be in any isotopic form, including ¹⁶O and ¹⁸O; F may be in any isotopic form, including ¹⁸F and ¹⁹F; and the like.

When describing the invention, which may include compounds and pharmaceutically acceptable salts thereof, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

In certain embodiments, the compounds disclosed herein comprise, for example, at least one hydrophilic head-group and at least one hydrophobic tail-group, each bound to at least one cleavable group, thereby rendering such compounds amphiphilic. As used herein to describe a compound or composition, the term “amphiphilic” means the ability to dissolve in both polar (e.g., water) and non-polar (e.g., lipid) environments. For example, in certain embodiments, the compounds disclosed herein comprise at least one lipophilic tail-group (e.g., cholesterol or a C₆-C₂₀ alkyl) and at least one hydrophilic head-group (e.g., imidazole), each bound to a cleavable group (e.g., disulfide).

It should be noted that the terms “head-group” and “tail-group” as used describe the compounds of the present invention, and in particular functional groups that comprise such compounds, are used for ease of reference to describe the orientation of one or more functional groups relative to other functional groups. For example, in certain embodiments a hydrophilic head-group (e.g., guanidinium) is bound (e.g., by one or more of hydrogen-bonds, van der Waals' forces, ionic interactions and covalent bonds) to a cleavable functional group (e.g., a disulfide group), which in turn is bound to a hydrophobic tail-group (e.g., cholesterol).

As used herein, the term “alkyl” refers to both straight and branched chain C₁-C₄₀ hydrocarbons (e.g., C₆-C₂₀ hydrocarbons), and include both saturated and unsaturated hydrocarbons. In certain embodiments, the alkyl may comprise one or more cyclic alkyls and/or one or more heteroatoms such as oxygen, nitrogen, or sulfur and may optionally be substituted with substituents (e.g., one or more of alkyl, halo, alkoxyl, hydroxy, amino, aryl, ether, ester or amide). In certain embodiments, a contemplated alkyl includes (9Z,12Z)-octadeca-9,12-dien. The use of designations such as, for example, “C₆-C₂₀” is intended to refer to an alkyl (e.g., straight or branched chain and inclusive of alkenes and alkyls) having the recited range carbon atoms. In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

As used herein, “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C2 alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

As used herein, “alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

As used herein, “alkylene,” “alkenylene,” and “alkynylene,” refer to a divalent radical of an alkyl, alkenyl, and alkynyl group respectively. When a range or number of carbons is provided for a particular “alkylene,” “alkenylene,” or “alkynylene,” group, it is understood that the range or number refers to the range or number of carbons in the linear carbon divalent chain. “Alkylene,” “alkenylene,” and “alkynylene,” groups may be substituted or unsubstituted with one or more substituents as described herein.

As used herein, the term “aryl” refers to aromatic groups (e.g., monocyclic, bicyclic and tricyclic structures) containing six to ten carbons in the ring portion. The aryl groups may be optionally substituted through available carbon atoms and in certain embodiments may include one or more heteroatoms such as oxygen, nitrogen or sulfur. In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl).

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclopentanes, cyclobutanes and cyclopropanes.

As used herein, “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” and “heterocyclic radical,” may be used interchangeably.

As used herein, “cyano” refers to —CN.

The terms “halo” and “halogen” as used herein refer to an atom selected from fluorine (fluoro, —F), chlorine (chloro, —Cl), bromine (bromo, —Br), and iodine (iodo, —I). In certain embodiments, the halo group is either fluoro or chloro.

The term “alkoxy,” as used herein, refers to an alkyl group which is attached to another moiety via an oxygen atom (—O(alkyl)). Non-limiting examples include e.g., methoxy, ethoxy, propoxy, and butoxy.

As used herein, “oxo” refers to —C═O.

In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

In typical embodiments, the present invention is intended to encompass the compounds disclosed herein, and the pharmaceutically acceptable salts, pharmaceutically acceptable esters, tautomeric forms, polymorphs, and prodrugs of such compounds. In some embodiments, the present invention includes a pharmaceutically acceptable addition salt, a pharmaceutically acceptable ester, a solvate (e.g., hydrate) of an addition salt, a tautomeric form, a polymorph, an enantiomer, a mixture of enantiomers, a stereoisomer or mixture of stereoisomers (pure or as a racemic or non-racemic mixture) of a compound described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

In certain embodiments the compounds and the transfer vehicles of which such compounds are a component (e.g., lipid nanoparticles) exhibit an enhanced (e.g., increased) ability to transfect one or more target cells. Accordingly, also provided herein are methods of transfecting one or more target cells. Such methods generally comprise the step of contacting the one or more target cells with the compounds and/or pharmaceutical compositions disclosed herein such that the one or more target cells are transfected with the circular RNA encapsulated therein. As used herein, the terms “transfect” or “transfection” refer to the intracellular introduction of one or more encapsulated materials (e.g., nucleic acids and/or polynucleotides) into a cell, or preferably into a target cell. The term “transfection efficiency” refers to the relative amount of such encapsulated material (e.g., polynucleotides) up-taken by, introduced into and/or expressed by the target cell which is subject to transfection. In some embodiments, transfection efficiency may be estimated by the amount of a reporter polynucleotide product produced by the target cells following transfection. In some embodiments, a transfer vehicle has high transfection efficiency. In some embodiments, a transfer vehicle has at least about 10%, 20%, 30%, 40%, 50%, 60%, 70%, 80%, or 90% transfection efficiency.

As used herein, the term “liposome” generally refers to a vesicle composed of lipids (e.g., amphiphilic lipids) arranged in one or more spherical bilayer or bilayers. In certain embodiments, the liposome is a lipid nanoparticle (e.g., a lipid nanoparticle comprising one or more of the ionizable lipid compounds disclosed herein). Such liposomes may be unilamellar or multilamellar vesicles which have a membrane formed from a lipophilic material and an aqueous interior that contains the encapsulated circRNA to be delivered to one or more target cells, tissues and organs. In certain embodiments, the compositions described herein comprise one or more lipid nanoparticles. Examples of suitable lipids (e.g., ionizable lipids) that may be used to form the liposomes and lipid nanoparticles contemplated include one or more of the compounds disclosed herein (e.g., HGT4001, HGT4002, HGT4003, HGT4004 and/or HGT4005). Such liposomes and lipid nanoparticles may also comprise additional ionizable lipids such as C12-200, DLin-KC2-DMA, and/or HGT5001, helper lipids, structural lipids, PEG-modified lipids, MC3, DLinDMA, DLinkC2DMA, cKK-E12, ICE, HGT5000, DODAC, DDAB, DMRIE, DOSPA, DOGS, DODAP, DODMA, DMDMA, DODAC, DLenDMA, DMRIE, CLinDMA, CpLinDMA, DMOBA, DOcarbDAP, DLinDAP, DLincarbDAP, DLinCDAP, KLin-K-DMA, DLin-K-XTC2-DMA, HGT4003, and combinations thereof.

As used herein, the phrases “non-cationic lipid”, “non-cationic helper lipid”, and “helper lipid” are used interchangeably and refer to any neutral, zwitterionic or anionic lipid.

As used herein, the phrase “anionic lipid” refers to any of a number of lipid species that carry a net negative charge at a selected pH, such as physiological pH.

As used herein, the phrase “biodegradable lipid” or “degradable lipid” refers to any of a number of lipid species that are broken down in a host environment on the order of minutes, hours, or days ideally making them less toxic and unlikely to accumulate in a host over time. Common modifications to lipids include ester bonds, and disulfide bonds among others to increase the biodegradability of a lipid.

As used herein, the phrase “biodegradable PEG lipid” or “degradable PEG lipid” refers to any of a number of lipid species where the PEG molecules are cleaved from the lipid in a host environment on the order of minutes, hours, or days ideally making them less immunogenic. Common modifications to PEG lipids include ester bonds, and disulfide bonds among others to increase the biodegradability of a lipid.

In certain embodiments of the present invention, the transfer vehicles (e.g., lipid nanoparticles) are prepared to encapsulate one or more materials or therapeutic agents (e.g., circRNA). The process of incorporating a desired therapeutic agent (e.g., circRNA) into a transfer vehicle is referred to herein as or “loading” or “encapsulating” (Lasic, et al., FEBS Lett., 312: 255-258, 1992). The transfer vehicle-loaded or -encapsulated materials (e.g., circRNA) may be completely or partially located in the interior space of the transfer vehicle, within a bilayer membrane of the transfer vehicle, or associated with the exterior surface of the transfer vehicle.

As used herein, the term “structural lipid” refers to sterols and also to lipids containing sterol moieties.

As defined herein, “sterols” are a subgroup of steroids consisting of steroid alcohols.

As used herein, the term “structural lipid” refers to sterols and also to lipids containing sterol moieties.

As used herein, the term “PEG” means any polyethylene glycol or other polyalkylene ether polymer.

As generally defined herein, a “PEG-OH lipid” (also referred to herein as “hydroxy-PEGylated lipid”) is a PEGylated lipid having one or more hydroxyl (—OH) groups on the lipid.

As used herein, a “phospholipid” is a lipid that includes a phosphate moiety and one or more carbon chains, such as unsaturated fatty acid chains.

All nucleotide sequences disclosed herein can represent an RNA sequence or a corresponding DNA sequence. It is understood that deoxythymidine (dT or T) in a DNA is transcribed into a uridine (U) in an RNA. As such, “T” and “U” are used interchangeably herein in nucleotide sequences.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

### 2. Vectors, Precursor RNA, and Circular RNA

Also provided herein are circular RNAs, precursor RNAs that can circularize into the circular RNAs, and vectors (e.g., DNA vectors) that can be transcribed into the precursor RNAs or the circular RNAs.

Two types of spacers have been designed for improving precursor RNA circularization and/or gene expression from circular RNA. The first type of spacer is external spacer, i.e., present in a precursor RNA but removed upon circularization. While not wishing to be bound by theory, it is contemplated that an external spacer may improve ribozyme-mediated circularization by maintaining the structure of the ribozyme itself and preventing other neighboring sequence elements from interfering with its folding and function. The second type of spacer is internal spacer, i.e., present in a precursor RNA and retained in a resulting circular RNA. While not wishing to be bound by theory, it is contemplated that an internal spacer may improve ribozyme-mediated circularization by maintaining the structure of the ribozyme itself and preventing other neighboring sequence elements, particularly the neighboring IRES and coding region, from interfering with its folding and function. It is also contemplated that an internal spacer may improve protein expression from the IRES by preventing neighboring sequence elements, particularly the intron elements, from hybridizing with sequences within the IRES and inhibiting its ability to fold into its most preferred and active conformation.

For driving protein expression, the circular RNA comprises an IRES operably linked to a protein coding sequence. Exemplary IRES sequences are provided in Table 17 below. In some embodiments, the circular RNA disclosed herein comprises an IRES sequence at least 80%, 85%, 90%, 91%, 92%, 93%, 94%, 95%, 96%, 97%, 98%, or 99% identical to an IRES sequence in Table 17. In some embodiments, the circular RNA disclosed herein comprises an IRES sequence in Table 17. Modifications of IRES and accessory sequences are disclosed herein to increase or reduce IRES activities, for example, by truncating the 5′ and/or 3′ ends of the IRES, adding a spacer 5′ to the IRES, modifying the 6 nucleotides 5′ to the translation initiation site (Kozak sequence), modification of alternative translation initiation sites, and creating chimeric/hybrid IRES sequences. In some embodiments, the IRES sequence in the circular RNA disclosed herein comprises one or more of these modifications relative to a native IRES (e.g., a native IRES disclosed in Table 17).

In certain aspects, provided herein are circular RNA polynucleotides comprising a 3′ post splicing group I intron fragment, optionally a first spacer, an Internal Ribosome Entry Site (IRES), an expression sequence, optionally a second spacer, and a 5′ post splicing group I intron fragment. In some embodiments, these regions are in that order. In some embodiments, the circular RNA is made by a method provided herein or from a vector provided herein.

In certain embodiments, transcription of a vector provided herein (e.g., comprising a 5′ homology region, a 3′ group I intron fragment, optionally a first spacer, an Internal Ribosome Entry Site (IRES), an expression sequence, optionally a second spacer, a 5′ group I intron fragment, and a 3′ homology region) results in the formation of a precursor linear RNA polynucleotide capable of circularizing. In some embodiments, this precursor linear RNA polynucleotide circularizes when incubated in the presence of guanosine nucleotide or nucleoside (e.g., GTP) and divalent cation (e.g., Mg²⁺).

In some embodiments, the vectors and precursor RNA polynucleotides provided herein comprise a first (5′) duplex forming region and a second (3′) duplex forming region. In certain embodiments, the first and second homology regions may form perfect or imperfect duplexes. Thus, in certain embodiments at least 75%, 80%, 85%, 90%, 91%, 92%, 93%, 94%, 95%, 96%, 97%, 98%, 99% or 100% of the first and second duplex forming regions may be base paired with one another. In some embodiments, the duplex forming regions are predicted to have less than 50% (e.g., less than 45%, less than 40%, less than 35%, less than 30%, less than 25%) base pairing with unintended sequences in the RNA (e.g., non-duplex forming region sequences). In some embodiments, including such duplex forming regions on the ends of the precursor RNA strand, and adjacent or very close to the group I intron fragment, bring the group I intron fragments in close proximity to each other, increasing splicing efficiency. In some embodiments, the duplex forming regions are 3 to 100 nucleotides in length (e.g., 3-75 nucleotides in length, 3-50 nucleotides in length, 20-50 nucleotides in length, 35-50 nucleotides in length, 5-25 nucleotides in length, 9-19 nucleotides in length). In some embodiments, the duplex forming regions are about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In some embodiments, the duplex forming regions have a length of about 9 to about 50 nucleotides. In one embodiment, the duplex forming regions have a length of about 9 to about 19 nucleotides. In some embodiments, the duplex forming regions have a length of about 20 to about 40 nucleotides. In certain embodiments, the duplex forming regions have a length of about 30 nucleotides.

In certain embodiments, the vectors, precursor RNA and circular RNA provided herein comprise a first (5′) and/or a second (3′) spacer. In some embodiments, including a spacer between the 3′ group I intron fragment and the IRES may conserve secondary structures in those regions by preventing them from interacting, thus increasing splicing efficiency. In some embodiments, the first (between 3′ group I intron fragment and IRES) and second (between the expression sequence and 5′ group I intron fragment) spacers comprise additional base pairing regions that are predicted to base pair with each other and not to the first and second duplex forming regions. In some embodiments, such spacer base pairing brings the group I intron fragments in close proximity to each other, further increasing splicing efficiency. Additionally, in some embodiments, the combination of base pairing between the first and second duplex forming regions, and separately, base pairing between the first and second spacers, promotes the formation of a splicing bubble containing the group I intron fragments flanked by adjacent regions of base pairing. Typical spacers are contiguous sequences with one or more of the following qualities: 1) predicted to avoid interfering with proximal structures, for example, the IRES, expression sequence, or intron; 2) is at least 7 nt long and no longer than 100 nt; 3) is located after and adjacent to the 3′ intron fragment and/or before and adjacent to the 5′ intron fragment; and 4) contains one or more of the following: a) an unstructured region at least 5 nt long, b) a region of base pairing at least 5 nt long to a distal sequence, including another spacer, and c) a structured region at least 7 nt long limited in scope to the sequence of the spacer. Spacers may have several regions, including an unstructured region, a base pairing region, a hairpin/structured region, and combinations thereof. In an embodiment, the spacer has a structured region with high GC content. In an embodiment, a region within a spacer base pairs with another region within the same spacer. In an embodiment, a region within a spacer base pairs with a region within another spacer. In an embodiment, a spacer comprises one or more hairpin structures. In an embodiment, a spacer comprises one or more hairpin structures with a stem of 4 to 12 nucleotides and a loop of 2 to 10 nucleotides. In an embodiment, there is an additional spacer between the 3′ group I intron fragment and the IRES. In an embodiment, this additional spacer prevents the structured regions of the IRES from interfering with the folding of the 3′ group I intron fragment or reduces the extent to which this occurs. In some embodiments, the 5′ spacer sequence is at least 7, 8, 9, 10, 11, 12, 13, 14, 15, 16, 17, 18, 19, 20, 25 or 30 nucleotides in length. In some embodiments, the 5′ spacer sequence is no more than 100, 90, 80, 70, 60, 50, 45, 40, 35 or 30 nucleotides in length. In some embodiments the 5′ spacer sequence is between 5 and 50, 10 and 50, 20 and 50, 20 and 40, and/or 25 and 35 nucleotides in length. In certain embodiments, the 5′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, the 5′ spacer sequence is a polyA sequence. In another embodiment, the 5′ spacer sequence is a polyAC sequence. In one embodiment, a spacer comprises about 10%, 20%, 30%, 40%, 50%, 60%, 70%, 80%, 90%, or 100% polyAC content. In one embodiment, a spacer comprises about 10%, 20%, 30%, 40%, 50%, 60%, 70%, 80%, 90%, or 100% polypyrimidine (C/T or C/U) content.

In certain embodiments, a 3′ group I intron fragment is a contiguous sequence at least 75% identical (e.g., at least 80%, 85%, 90%, 91%, 92%, 93%, 94%, 95%, 96%, 97%, 98%, 99% or 100% identical) to a 3′ proximal fragment of a natural group I intron including the 3′ splice site dinucleotide and optionally the adjacent exon sequence at least 1 nt in length (e.g., at least 2, 3, 4, 5, 6, 7, 8, 9, 10, 15, 20, 25 or 30 nt in length) and at most the length of the exon. Typically, a 5′ group I intron fragment is a contiguous sequence at least 75% identical (e.g., at least 80%, 85%, 90%, 91%, 92%, 93%, 94%, 95%, 96%, 97%, 98%, 99% or 100% identical) to a 5′ proximal fragment of a natural group I intron including the 5′ splice site dinucleotide and optionally the adjacent exon sequence at least 1 nt in length (e.g., at least 2, 3, 4, 5, 6, 7, 8, 9, 10, 15, 20, 25 or 30 nt in length) and at most the length of the exon. As described by Umekage et al. (2012), external portions of the 3′ group I intron fragment and 5′ group I intron fragment are removed in circularization, causing the circular RNA provided herein to comprise only the portion of the 3′ group I intron fragment formed by the optional exon sequence of at least 1 nt in length and 5′ group I intron fragment formed by the optional exon sequence of at least 1 nt in length, if such sequences were present on the non-circularized precursor RNA. The part of the 3′ group I intron fragment that is retained by a circular RNA is referred to herein as the post splicing 3′ group I intron fragment. The part of the 5′ group I intron fragment that is retained by a circular RNA is referred to herein as the post splicing 5′ group I intron fragment.

In certain embodiments, the vectors, precursor RNA and circular RNA provided herein comprise an internal ribosome entry site (IRES). Inclusion of an IRES permits the translation of one or more open reading frames from a circular RNA (e.g., open reading frames that form the expression sequence). The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20: 102-110; Kobayashi et al., BioTechniques (1996) 21:399-402; and Mosser et al., BioTechniques 1997 22 150-161).

A multitude of IRES sequences are available and include sequences derived from a wide variety of viruses, such as from leader sequences of picornaviruses such as the encephalomyocarditis virus (EMCV) UTR (fang et al. J. Virol. (1989) 63: 1651-1660), the polio leader sequence, the hepatitis A virus leader, the hepatitis C virus IRES, human rhinovirus type 2 IRES (Dobrikova et al., Proc. Natl. Acad. Sci. (2003) 100(25): 15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279(5):3389-3397), and the like.

In some embodiments, the IRES is an IRES sequence of Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Himetobi P virus, Hepatitis C virus, Hepatitis A virus, Hepatitis GB virus, Foot and mouth disease virus, Human enterovirus 71, Equine rhinitis virus, Ectropis obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human eIF4G, Mouse NDST4L, Human LEF1, Mouse HIF1 alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SH1, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picorna-like Virus, CRPV, Salivirus A BNS, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24 or an aptamer to eIF4G.

In some embodiments, the polynucleotides herein comprise an expression sequence. In some embodiments, the expression sequence encodes a therapeutic protein.

In some embodiments, the circular RNA encodes two or more polypeptides. In some embodiments, the circular RNA is a bicistronic RNA. The sequences encoding the two or more polypeptides can be separated by a ribosomal skipping element or a nucleotide sequence encoding a protease cleavage site. In certain embodiments, the ribosomai skipping element encodes thosea-asigna virus 2A peptide (T2A), porcine teschovirus-1 2 A peptide (P2A), foot-and-mouth disease virus 2 A peptide (F2A), equine rhinitis A vims 2A peptide (E2A), cytoplasmic polyhedrosis vims 2A peptide (BmCPV 2A), or flacherie vims of B. mori 2A peptide (BmIFV 2A).

In certain embodiments, the vectors provided herein comprise a 3′ UTR. In some embodiments, the 3′ UTR is from human beta globin, human alpha globin Xenopus beta globin, Xenopus alpha globin, human prolactin, human GAP-43, human eEFlal, human Tau, human TNFα, dengue virus, hantavirus small mRNA, bunyavirus small mRNA, turnip yellow mosaic virus, hepatitis C virus, rubella virus, tobacco mosaic virus, human IL-8, human actin, human GAPDH, human tubulin, hibiscus chlorotic ringspot virus, woodchuck hepatitis virus post translationally regulated element, sindbis virus, turnip crinkle virus, tobacco etch virus, or Venezuelan equine encephalitis virus.

In some embodiments, the vectors provided herein comprise a 5′ UTR. In some embodiments, the 5′ UTR is from human beta globin, Xenopus laevis beta globin, human alpha globin, Xenopus laevis alpha globin, rubella virus, tobacco mosaic virus, mouse Gtx, dengue virus, heat shock protein 70 kDa protein 1A, tobacco alcohol dehydrogenase, tobacco etch virus, turnip crinkle virus, or the adenovirus tripartite leader.

In some embodiments, a vector provided herein comprises a polyA region external of the 3′ and/or 5′ group I intron fragments. In some embodiments the polyA region is at least 15, 30, or 60 nucleotides long. In some embodiments, one or both polyA regions is 15-50 nucleotides long. In some embodiments, one or both polyA regions is 20-25 nucleotides long. The polyA sequence is removed upon circularization. Thus, an oligonucleotide hybridizing with the polyA sequence, such as a deoxythymine oligonucleotide (oligo(dT)) conjugated to a solid surface (e.g., a resin), can be used to separate circular RNA from its precursor RNA. Other sequences can also be disposed 5′ to the 3′ group I intron fragment or 3′ to the 5′ group I intron fragment and a complementary sequence can similarly be used for circular RNA purification.

In some embodiments, the DNA (e.g., vector), linear RNA (e.g., precursor RNA), and/or circular RNA polynucleotide provided herein is between 300 and 10000, 400 and 9000, 500 and 8000, 600 and 7000, 700 and 6000, 800 and 5000, 900 and 5000, 1000 and 5000, 1100 and 5000, 1200 and 5000, 1300 and 5000, 1400 and 5000, and/or 1500 and 5000 nucleotides in length. In some embodiments, the polynucleotide is at least 300 nt, 400 nt, 500 nt, 600 nt, 700 nt, 800 nt, 900 nt, 1000 nt, 1100 nt, 1200 nt, 1300 nt, 1400 nt, 1500 nt, 2000 nt, 2500 nt, 3000 nt, 3500 nt, 4000 nt, 4500 nt, or 5000 nt in length. In some embodiments, the polynucleotide is no more than 3000 nt, 3500 nt, 4000 nt, 4500 nt, 5000 nt, 6000 nt, 7000 nt, 8000 nt, 9000 nt, or 10000 nt in length. In some embodiments, the length of a DNA, linear RNA, and/or circular RNA polynucleotide provided herein is about 300 nt, 400 nt, 500 nt, 600 nt, 700 nt, 800 nt, 900 nt, 1000 nt, 1100 nt, 1200 nt, 1300 nt, 1400 nt, 1500 nt, 2000 nt, 2500 nt, 3000 nt, 3500 nt, 4000 nt, 4500 nt, 5000 nt, 6000 nt, 7000 nt, 8000 nt, 9000 nt, or 10000 nt.

In some embodiments, provided herein is a vector. In certain embodiments, the vector comprises, in the following order, a) a 5′ homology region, b) a 3′ group I intron fragment, c) optionally, a first spacer sequence, d) an IRES, e) an expression sequence, f) optionally, a second spacer sequence, g) a 5′ group I intron fragment, and h) a 3′ homology region. In some embodiments, the vector comprises a transcriptional promoter upstream of the 5′ homology region. In certain embodiments, the precursor RNA comprises, in the following order, a) a polyA sequence, b) an external spacer, c) a 3′ group I intron fragment, d) a duplex forming region, e) an internal spacer, f) an IRES, g) an expression sequence, h) a stop codon cassette, i) optionally, an internal spacer, j) a duplex forming region capable of forming a duplex with the duplex forming region of d, k) a 5′ group I intron fragment, l) an external spacer, and m) a polyA sequence.

In some embodiments, provided herein is a precursor RNA. In certain embodiments, the precursor RNA is a linear RNA produced by in vitro transcription of a vector provided herein. In some embodiments, the precursor RNA comprises, in the following order, a) a 5′ homology region, b) a 3′ group I intron fragment, c) optionally, a first spacer sequence, d) an IRES, e) an expression sequence, f) optionally, a second spacer sequence, g) a 5′ group I intron fragment, and h) a 3′ homology region. The precursor RNA can be unmodified, partially modified or completely modified.

In certain embodiments, provided herein is a circular RNA. In certain embodiments, the circular RNA is a circular RNA produced by a vector provided herein. In some embodiments, the circular RNA is circular RNA produced by circularization of a precursor RNA provided herein. In some embodiments, the circular RNA comprises, in the following sequence, a) a first spacer sequence, b) an IRES, c) an expression sequence, and d) a second spacer sequence. In some embodiments, the circular RNA further comprises the portion of the 3′ group I intron fragment that is 3′ of the 3′ splice site. In some embodiments, the circular RNA further comprises the portion of the 5′ group I intron fragment that is 5′ of the 5′ splice site. In some embodiments, the circular RNA is at least 500, 600, 700, 800, 900, 1000, 1500, 2000, 2500, 3000, 3500, 4000 or 4500 nucleotides in size. The circular RNA can be unmodified, partially modified or completely modified.

In some embodiments, the circular RNA provided herein has higher functional stability than mRNA comprising the same expression sequence. In some embodiments, the circular RNA provided herein has higher functional stability than mRNA comprising the same expression sequence, 5moU modifications, an optimized UTR, a cap, and/or a polyA tail.

In some embodiments, the circular RNA polynucleotide provided herein has a functional half-life of at least 5 hours, 10 hours, 15 hours, 20 hours. 30 hours, 40 hours, 50 hours, 60 hours, 70 hours or 80 hours. In some embodiments, the circular RNA polynucleotide provided herein has a functional half-life of 5-80, 10-70, 15-60, and/or 20-50 hours. In some embodiments, the circular RNA polynucleotide provided herein has a functional half-life greater than (e.g., at least 1.5-fold greater than, at least 2-fold greater than) that of an equivalent linear RNA polynucleotide encoding the same protein. In some embodiments, functional half-life can be assessed through the detection of functional protein synthesis.

In some embodiments, the circular RNA polynucleotide provided herein has a half-life of at least 5 hours, 10 hours, 15 hours, 20 hours. 30 hours, 40 hours, 50 hours, 60 hours, 70 hours or 80 hours. In some embodiments, the circular RNA polynucleotide provided herein has a half-life of 5-80, 10-70, 15-60, and/or 20-50 hours. In some embodiments, the circular RNA polynucleotide provided herein has a half-life greater than (e.g., at least 1.5-fold greater than, at least 2-fold greater than) that of an equivalent linear RNA polynucleotide encoding the same protein. In some embodiments, the circular RNA polynucleotide, or pharmaceutical composition thereof, has a functional half-life in a human cell greater than or equal to that of a pre-determined threshold value. In some embodiments the functional half-life is determined by a functional protein assay. For example in some embodiments, the functional half-life is determined by an in vitro luciferase assay, wherein the activity of Gaussia luciferase (GLuc) is measured in the media of human cells (e.g. HepG2) expressing the circular RNA polynucleotide every 1, 2, 6, 12, or 24 hours over 1, 2, 3, 4, 5, 6, 7, or 14 days. In other embodiments, the functional half-life is determined by an in vivo assay, wherein levels of a protein encoded by the expression sequence of the circular RNA polynucleotide are measured in patient serum or tissue samples every 1, 2, 6, 12, or 24 hours over 1, 2, 3, 4, 5, 6, 7, or 14 days. In some embodiments, the pre-determined threshold value is the functional half-life of a reference linear RNA polynucleotide comprising the same expression sequence as the circular RNA polynucleotide.

In some embodiments, the circular RNA provided herein may have a higher magnitude of expression than equivalent linear mRNA, e.g., a higher magnitude of expression 24 hours after administration of RNA to cells. In some embodiments, the circular RNA provided herein has a higher magnitude of expression than mRNA comprising the same expression sequence, 5moU modifications, an optimized UTR, a cap, and/or a polyA tail.

In some embodiments, the circular RNA provided herein may be less immunogenic than an equivalent mRNA when exposed to an immune system of an organism or a certain type of immune cell. In some embodiments, the circular RNA provided herein is associated with modulated production of cytokines when exposed to an immune system of an organism or a certain type of immune cell. For example, in some embodiments, the circular RNA provided herein is associated with reduced production of IFN-β1, RIG-I, IL-2, IL-6, IFNγ, and/or TNFα when exposed to an immune system of an organism or a certain type of immune cell as compared to mRNA comprising the same expression sequence. In some embodiments, the circular RNA provided herein is associated with less IFN-β1, RIG-I, IL-2, IL-6, IFNγ, and/or TNFα transcript induction when exposed to an immune system of an organism or a certain type of immune cell as compared to mRNA comprising the same expression sequence. In some embodiments, the circular RNA provided herein is less immunogenic than mRNA comprising the same expression sequence. In some embodiments, the circular RNA provided herein is less immunogenic than mRNA comprising the same expression sequence, 5moU modifications, an optimized UTR, a cap, and/or a polyA tail.

In certain embodiments, the circular RNA provided herein can be transfected into a cell as is, or can be transfected in DNA vector form and transcribed in the cell. Transcription of circular RNA from a transfected DNA vector can be via added polymerases or polymerases encoded by nucleic acids transfected into the cell, or preferably via endogenous polymerases.

In certain embodiments, a circular RNA polynucleotide provided herein comprises modified RNA nucleotides and/or modified nucleosides. In some embodiments, the modified nucleoside is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine). In other embodiments, the modified nucleoside is m¹A (1-methyladenosine); m²A (2-methyladenosine); Am (2′-O-methyladenosine); ms² m⁶A (2-methylthio-N⁶-methyladenosine); i⁶A (N⁶-isopentenyladenosine); ms²i6A (2-methylthio-N⁶ isopentenyladenosine); io⁶A (N⁶-(cis-hydroxyisopentenyl)adenosine); ms²io⁶A (2-methylthio-N⁶-(cis-hydroxyisopentenyl)adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m² ₂G (N²,N²-dimethylguanosine); m²Gm (N²,2′-O-dimethylguanosine); m² ₂Gm (N²,N²,2′-O-trimethylguanosine); Gr(p) (2′-O-ribosylguanosine(phosphate)); yW (wybutosine); o2yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵ s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵S²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶ ₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶ ₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm ⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In some embodiments, the modified nucleoside may include a compound selected from the group of: pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In another embodiment, the modifications are independently selected from the group consisting of 5-methylcytosine, pseudouridine and 1-methylpseudouridine.

In some embodiments, the modified ribonucleosides include 5-methylcytidine, 5-methoxyuridine, 1-methyl-pseudouridine, N6-methyladenosine, and/or pseudouridine. In some embodiments, such modified nucleosides provide additional stability and resistance to immune activation.

In particular embodiments, polynucleotides may be codon-optimized. A codon optimized sequence may be one in which codons in a polynucleotide encoding a polypeptide have been substituted in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid. In some embodiments, a codon optimized polynucleotide may minimize ribozyme collisions and/or limit structural interference between the expression sequence and the IRES.

In certain embodiments circular RNA provided herein is produced inside a cell. In some embodiments, precursor RNA is transcribed using a DNA template (e.g., in some embodiments, using a vector provided herein) in the cytoplasm by a bacteriophage RNA polymerase, or in the nucleus by host RNA polymerase II and then circularized.

In certain embodiments, the circular RNA provided herein is injected into an animal (e.g., a human), such that a polypeptide encoded by the circular RNA molecule is expressed inside the animal.

### 3. Payload

In some embodiments, the expression sequence encodes a therapeutic protein. In some embodiments, the therapeutic protein is selected from the proteins listed in the following table.

| | | Target | |
|---|---|---|---|
| | | cell/ | |
| Payload | Sequence | organ | Preferred delivery formulation |
| CD19 CAR | Any of sequences 309-314 | T cells | 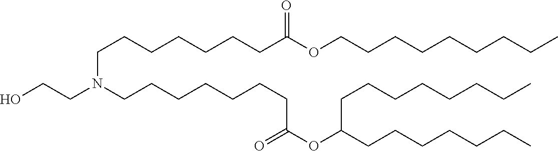 [SMILES: CCCCCCCCCOC(=O)CCCCCCCN(CCO)CCCCCCCC(=O)OC(CCCCCCC)CCCCCCCC] |
| | | | (50 mol %) |
| | | | DSPC (10 mol %) |
| | | | Beta-sitosterol (28.5% mol %) |
| | | | Cholesterol (10 mol %) |
| | | | PEG DMG (1.5mol %) |
| BCMA CAR | MALPVTALLLPLALLLHAAR PDIVLTQSPASLAVSLGERAT INCRASESVSVIGAHLIHWY QQKPGQPPKLLIYLASNLET GVPARFSGSGSGTDFTLTISS LQAEDAAIYYCLQSRIFPRTF GQGTKLEIKGSTSGSGKPGS GEGSTKGQVQLVQSGSELK KPGASVKVSCKASGYTFTD YSINWVRQAPGQGLEWMG | T cells | 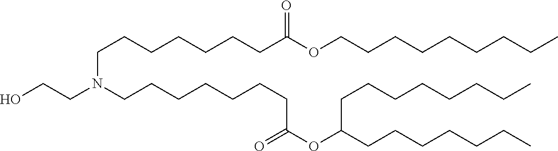 [SMILES: CCCCCCCCCOC(=O)CCCCCCCN(CCO)CCCCCCCC(=O)OC(CCCCCCC)CCCCCCCC] |
| | WINTETREPAYAYDFRGRFV | | (50 mol %) |
| | FSLDTSVSTAYLQISSLKAED | | DSPC (10 mol %) |
| | TAVYYCARDYSYAMDYWG | | Beta-sitosterol (28.5% mol %) |
| | QGTLVTVSSAAATTTPAPRP | | Cholesterol (10 mol %) |
| | PTPAPTIASQPLSLRPEACRP | | PEG DMG (1.5 mol %) |
| | AAGGAVHTRGLDFACDIYI | | |
| | WAPLAGTCGVLLLSLVITLY | | |
| | CKRGRKKLLYIFKQPFMRPV | | |
| | QTTQEEDGCSCRFPEEEEGG | | |
| | CELRVKFSRSADAPAYQQG | | |
| | QNQLYNELNLGRREEYDVL | | |
| | DKRRGRDPEMGGKPRRKNP | | |
| | QEGLYNELQKDKMAEAYSE | | |
| | IGMKGERRRGKGHDGLYQG | | |
| | LSTATKDTYDALHMQALPPR | | |
| MAGE- A4 TCR | TCR alpha chain: KNQVEQSPQSLIILEGKNCTL QCNYTVSPFSNLRWYKQDT GRGPVSLTIMTFSENTKSNG RYTATLDADTKQSSLHITAS QLSDSASYICVVNHSGGSYIP TFGRGTSLIVHPYIQKPDPAV YQLRDSKSSDKSVCLFTDFD SQTNVSQSKDSDVYITDKTV LDMRSMDFKSNSAVAWSNK | T cells | 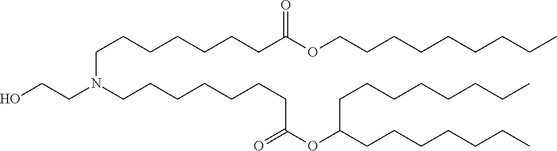 [SMILES: CCCCCCCCCOC(=O)CCCCCCCN(CCO)CCCCCCCC(=O)OC(CCCCCCC)CCCCCCCC] |
| | SDFACANAFNNSIIPEDTFFP | | (50 mol %) |
| | SPESS | | DSPC (10 mol %) |
| | TCR beta chain: | | Beta-sitosterol (28.5% mol %) |
| | DVKVTQSSRYLVKRTGEKV | | Cholesterol (10 mol %) |
| | FLECVQDMDHENMFWYRQ | | PEG DMG (1.5 mol %) |
| | DPGLGLRLIYFSYDVKMKEK | | |
| | GDIPEGYSVSREKKERFSLIL | | |
| | ESASTNQTSMYLCASSFLMT | | |
| | SGDPYEQYFGPGTRLTVTED | | |
| | LKNVFPPEVAVFEPSEAEISH | | |
| | TQKATLVCLATGFYPDHVEL | | |
| | SWWVNGKEVHSGVSTDPQP | | |
| | LKEQPALNDSRYCLSSRLRV | | |
| | SATFWQNPRNHFRCQVQFY | | |
| | GLSENDEWTQDRAKPVTQI | | |
| | VSAEAWGRAD | | |
| NY-ESO TCR | TCRalpha extracellular sequence MQEVTQIPAALSVPEGENLV LNCSFTDSAIYNLQWFRQDP GKGLTSLLLIQSSQREQTSGR LNASLDKSSGRSTLYIAASQP GDSATYLCAVRPTSGGSYIP TFGRGTSLIVHPY TCRbeta extracellular sequence MGVTQTPKFQVLKTGQSMT LQCAQDMNHEYMSWYRQD | T cells | 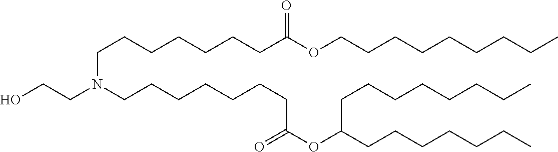 [SMILES: CCCCCCCCCOC(=O)CCCCCCCN(CCO)CCCCCCCC(=O)OC(CCCCCCC)CCCCCCCC] |
| | PGMGLRLIHYSVGAGITDQG | | (50 mol %) |
| | EVPNGYNVSRSTTEDFPLRL | | DSPC (10 mol %) |
| | LSAAPSQTSVYFCASSYVGN | | Beta-sitosterol (28.5% mol %) |
| | TGELFFGEGSRLTVL | | Cholesterol (10 mol %) |
| | | | PEG DMG (1.5 mol %) |
| EPO | APPRLICDSRVLERYLLEAKE | Kidney | |
| | AENITTGCAEHCSLNENITVP | or bone | |
| | DTKVNFYAWKRMEVGQQA | marrow | |
| | VEVWQGLALLSEAVLRGQA | | |
| | LLVNSSQPWEPLQLHVDKA | | |
| | VSGLRSLTTLLRALGAQKEA | | |
| | ISPPDAASAAPLRTITADTFR | | |
| | KLFRVYSNFLRGKLKLYTGE | | |
| | ACRTGDR | | |
| PAH | MSTAVLENPGLGRKLSDFG QETSYIEDNCNQNGAISLIFS LKEEVGALAKVLRLFEEND VNLTHIESRPSRLKKDEYEFF THLDKRSLPALTNIIKILRHDI GATVHELSRDKKKDTVPWF PRTIQELDRFANQILSYGAEL DADHPGFKDPVYRARRKQF ADIAYNYRHGQPIPRVEYME EEKKTWGTVFKTLKSLYKT | Hepatic cells | 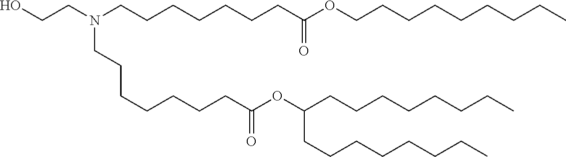 [SMILES: CCCCCCCCCOC(=O)CCCCCCCN(CCO)CCCCCCCC(=O)OC(CCCCCCCC)CCCCCCCC] |
| | HACYEYNHIFPLLEKYCGFH | | |
| | EDNIPQLEDVSQFLQTCTGF | | (50 mol %) |
| | RLRPVAGLLSSRDFLGGLAF | | DSPC (10 mol %) |
| | RVFHCTQYIRHGSKPMYTPE | | Cholesterol (38.5% mol %) |
| | PDICHELLGHVPLFSDRSFAQ | | PEG-DMG (1.5%) |
| | FSQEIGLASLGAPDEYIEKLA | | OR |
| | TIYWFTVEFGLCKQGDSIKA | | MC3 (50 mol %) |
| | YGAGLLSSFGELQYCLSEKP | | DSPC (10 mol %) |
| | KLLPLELEKTAIQNYTVTEF | | Cholesterol (38.5% mol %) |
| | QPLYYVAESFNDAKEKVRN | | PEG-DMG (1.5%) |
| | FAATIPRPFSVRYDPYTQRIE | | |
| | VLDNTQQLKILADSINSEIGI | | |
| | LCSALQKIK | | |
| CPS1 | LSVKAQTAHIVLEDGTKMK GYSFGHPSSVAGEVVFNTGL GGYPEAITDPAYKGQILTMA NPIIGNGGAPDTTALDELGLS KYLESNGIKVSGLLVLDYSK DYNHWLATKSLGQWLQEE KVPAIYGVDTRMLTKIIRDK GTMLGKIEFEGQPVDFVDPN KQNLIAEVSTKDVKVYGKG NPTKVVAVDCGIKNNVIRLL | Hepatic cells | 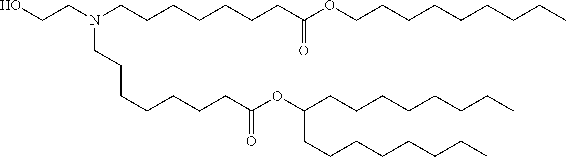 [SMILES: CCCCCCCCCOC(=O)CCCCCCCN(CCO)CCCCCCCC(=O)OC(CCCCCCCC)CCCCCCCC] |
| | VKRGAEVHLVPWNHDFTK | | |
| | MEYDGILIAGGPGNPALAEP | | (50 mol %) |
| | LIQNVRKILESDRKEPLFGIST | | DSPC (10 mol %) |
| | GNLITGLAAGAKTYKMSMA | | Cholesterol (38.5% mol %) |
| | NRGQNQPVLNITNKQAFITA | | PEG-DMG (1.5%) |
| | QNHGYALDNTLPAGWKPLF | | OR |
| | VNVNDQTNEGIMHESKPFFA | | MC3 (50 mol %) |
| | VQFHPEVTPGPIDTEYLFDSF | | DSPC (10 mol %) |
| | FSLIKKGKATTITSVLPKPAL | | Cholesterol (38.5% mol %) |
| | VASRVEVSKVLILGSGGLSIG | | PEG-DMG (1.5%) |
| | QAGEFDYSGSQAVKAMKEE | | |
| | NVKTVLMNPNIASVQTNEV | | |
| | GLKQADTVYFLPITPQFVTE | | |
| | VIKAEQPDGLILGMGGQTAL | | |
| | NCGVELFKRGVLKEYGVKV | | |
| | LGTSVESIMATEDRQLFSDK | | |
| | LNEINEKIAPSFAVESIEDAL | | |
| | KAADTIGYPVMIRSAYALGG | | |
| | LGSGICPNRETLMDLSTKAF | | |
| | AMTNQILVEKSVTGWKEIEY | | |
| | EVVRDADDNCVTVCNMEN | | |
| | VDAMGVHTGDSVVVAPAQ | | |
| | TLSNAEFQMLRRTSINVVRH | | |
| | LGIVGECNIQFALHPTSMEY | | |
| | CIIEVNARLSRSSALASKATG | | |
| | YPLAFIAAKIALGIPLPEIKNV | | |
| | VSGKTSACFEPSLDYMVTKI | | |
| | PRWDLDRFHGTSSRIGSSMK | | |
| | SVGEVMAIGRTFEESFQKAL | | |
| | RMCHPSIEGFTPRLPMNKEW | | |
| | PSNLDLRKELSEPSSTRIYAI | | |
| | AKAIDDNMSLDEIEKLTYID | | |
| | KWFLYKMRDILNMEKTLKG | | |
| | LNSESMTEETLKRAKEIGFS | | |
| | DKQISKCLGLTEAQTRELRL | | |
| | KKNIHPWVKQIDTLAAEYPS | | |
| | VTNYLYVTYNGQEHDVNFD | | |
| | DHGMMVLGCGPYHIGSSVE | | |
| | FDWCAVSSIRTLRQLGKKTV | | |
| | VVNCNPETVSTDFDECDKLY | | |
| | FEELSLERILDIYHQEACGGC | | |
| | IISVGGQIPNNLAVPLYKNGV | | |
| | KIMGTSPLQIDRAEDRSIFSA | | |
| | VLDELKVAQAPWKAVNTLN | | |
| | EALEFAKSVDYPCLLRPSYV | | |
| | LSGSAMNVVFSEDEMKKFL | | |
| | EEATRVSQEHPVVLTKFVEG | | |
| | AREVEMDAVGKDGRVISHA | | |
| | ISEHVEDAGVHSGDATLMLP | | |
| | TQTISQGAIEKVKDATRKIA | | |
| | KAFAISGPFNVQFLVKGNDV | | |
| | LVIECNLRASRSFPFVSKTLG | | |
| | VDFIDVATKVMIGENVDEK | | |
| | HLPTLDHPIIPADYVAIKAPM | | |
| | FSWPRLRDADPILRCEMAST | | |
| | GEVACFGEGIHTAFLKAMLS | | |
| | TGFKIPQKGILIGIQQSFRPRF | | |
| | LGVAEQLHNEGFKLFATEAT | | |
| | SDWLNANNVPATPVAWPSQ | | |
| | EGQNPSLSSIRKLIRDGSIDL | | |
| | VINLPNNNTKFVHDNYVIRR | | |
| | TAVDSGIPLLTNFQVTKLFA | | |
| | EAVQKSRKVDSKSLFHYRQ | | |
| | YSAGKAA | | |
| Cas9 | MKRNYILGLDIGITSVGYGII DYETRDVIDAGVRLFKEAN VENNEGRRSKRGARRLKRR RRHRIQRVKKLLFDYNLLTD HSELSGINPYEARVKGLSQK LSEEEFSAALLHLAKRRGVH NVNEVEEDTGNELSTKEQIS RNSKALEEKYVAELQLERLK KDGEVRGSINRFKTSDYVKE AKQLLKVQKAYHQLDQSFI | Immune cells | 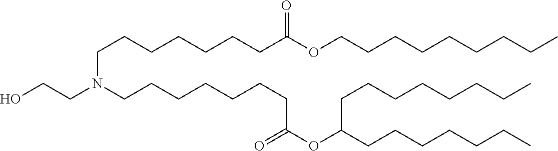 [SMILES: CCCCCCCCCOC(=O)CCCCCCCN(CCO)CCCCCCCC(=O)OC(CCCCCCC)CCCCCCCC] |
| | DTYIDLLETRRTYYEGPGEG | | (50 mol %) |
| | SPFGWKDIKEWYEMLMGHC | | DSPC (10 mol %) |
| | TYFPEELRSVKYAYNADLY | | Beta-sitosterol (28.5% mol %) |
| | NALNDLNNLVITRDENEKLE | | Cholesterol (10 mol %) |
| | YYEKFQIIENVFKQKKKPTL | | PEG DMG (1.5 mol %) |
| | KQIAKEILVNEEDIKGYRVTS | | |
| | TGKPEFTNLKVYHDIKDITA | | |
| | RKEIIENAELLDQIAKILTIYQ | | |
| | SSEDIQEELTNLNSELTQEEIE | | |
| | QISNLKGYTGTHNLSLKAIN | | |
| | LILDELWHTNDNQIAIFNRL | | |
| | KLVPKKVDLSQQKEIPTTLV | | |
| | DDFILSPVVKRSFIQSIKVINA | | |
| | IIKKYGLPNDIIIELAREKNSK | | |
| | DAQKMINEMQKRNRQTNER | | |
| | IEEIIRTTGKENAKYLIEKIKL | | |
| | HDMQEGKCLYSLEAIPLEDL | | |
| | LNNPFNYEVDHIIPRSVSFDN | | |
| | SFNNKVLVKQEENSKKGNR | | |
| | TPFQYLSSSDSKISYETFKKH | | |
| | ILNLAKGKGRISKTKKEYLL | | |
| | EERDINRFSVQKDFINRNLV | | |
| | DTRYATRGLMNLLRSYFRV | | |
| | NNLDVKVKSINGGFTSFLRR | | |
| | KWKFKKERNKGYKHHAED | | |
| | ALIIANADFIFKEWKKLDKA | | |
| | KKVMENQMFEEKQAESMPE | | |
| | IETEQEYKEIFITPHQIKHIKD | | |
| | FKDYKYSHRVDKKPNRELIN | | |
| | DTLYSTRKDDKGNTLIVNNL | | |
| | NGLYDKDNDKLKKLINKSPE | | |
| | KLLMYHHDPQTYQKLKLIM | | |
| | EQYGDEKNPLYKYYEETGN | | |
| | YLTKYSKKDNGPVIKKIKYY | | |
| | GNKLNAHLDITDDYPNSRN | | |
| | KVVKLSLKPYRFDVYLDNG | | |
| | VYKFVTVKNLDVIKKENYY | | |
| | EVNSKCYEEAKKLKKISNQA | | |
| | EFIASFYNNDLIKINGELYRV | | |
| | IGVNNDLLNRIEVNMIDITYR | | |
| | EYLENMNDKRPPRIIKTIASK | | |
| | TQSIKKYSTDILGNLYEVKS | | |
| | KKHPQIIKKG | | |
| ADAMTS 13 | AAGGILHLELLVAVGPDVFQ AHQEDTERYVLTNLNIGAEL LRDPSLGAQFRVHLVKMVIL TEPEGAPNITANLTSSLLSVC GWSQTINPEDDTDPGHADL VLYITRFDLELPDGNRQVRG VTQLGGACSPTWSCLITEDT GFDLGVTIAHEIGHSFGLEH DGAPGSGCGPSGHVMASDG AAPRAGLAWSPCSRRQLLSL | Hepatic cells | 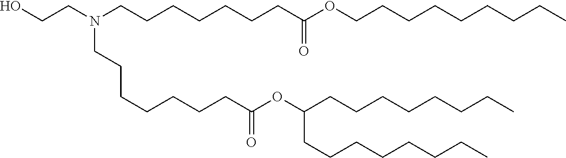 [SMILES: CCCCCCCCCOC(=O)CCCCCCCN(CCO)CCCCCCCC(=O)OC(CCCCCCCC)CCCCCCCC] |
| | LSAGRARCVWDPPRPQPGS | | |
| | AGHPPDAQPGLYYSANEQC | | (50 mol %) |
| | RVAFGPKAVACTFAREHLD | | DSPC (10 mol %) |
| | MCQALSCHTDPLDQSSCSRL | | Cholesterol (38.5% mol %) |
| | LVPLLDGTECGVEKWCSKG | | PEG-DMG (1.5%) |
| | RCRSLVELTPIAAVHGRWSS | | OR |
| | WGPRSPCSRSCGGGVVTRR | | MC3 (50 mol %) |
| | RQCNNPRPAFGGRACVGAD | | DSPC (10 mol %) |
| | LQAEMCNTQACEKTQLEFM | | Cholesterol (38.5% mol %) |
| | SQQCARTDGQPLRSSPGGAS | | PEG-DMG (1.5%) |
| | FYHWGAAVPHSQGDALCRH | | |
| | MCRAIGESFIMKRGDSFLDG | | |
| | TRCMPSGPREDGTLSLCVSG | | |
| | SCRTFGCDGRMDSQQVWDR | | |
| | CQVCGGDNSTCSPRKGSFTA | | |
| | GRAREYVTFLTVTPNLTSVY | | |
| | IANHRPLFTHLAVRIGGRYV | | |
| | VAGKMSISPNTTYPSLLEDG | | |
| | RVEYRVALTEDRLPRLEEIRI | | |
| | WGPLQEDADIQVYRRYGEE | | |
| | YGNLTRPDITFTYFQPKPRQ | | |
| | AWVWAAVRGPCSVSCGAG | | |
| | LRWVNYSCLDQARKELVET | | |
| | VQCQGSQQPPAWPEACVLE | | |
| | PCPPYWAVGDFGPCSASCG | | |
| | GGLRERPVRCVEAQGSLLKT | | |
| | LPPARCRAGAQQPAVALETC | | |
| | NPQPCPARWEVSEPSSCTSA | | |
| | GGAGLALENETCVPGADGL | | |
| | EAPVTEGPGSVDEKLPAPEP | | |
| | CVGMSCPPGWGHLDATSAG | | |
| | EKAPSPWGSIRTGAQAAHV | | |
| | WTPAAGSCSVSCGRGLMEL | | |
| | RFLCMDSALRVPVQEELCGL | | |
| | ASKPGSRREVCQAVPCPAR | | |
| | WQYKLAACSVSCGRGVVRR | | |
| | ILYCARAHGEDDGEEILLDT | | |
| | QCQGLPRPEPQEACSLEPCPP | | |
| | RWKVMSLGPCSASCGLGTA | | |
| | RRSVACVQLDQGQDVEVDE | | |
| | AACAALVRPEASVPCLIADC | | |
| | TYRWHVGTWMECSVSCGD | | |
| | GIQRRRDTCLGPQAQAPVPA | | |
| | DFCQHLPKPVTVRGCWAGP | | |
| | CVGQGTPSLVPHEEAAAPGR | | |
| | TTATPAGASLEWSQARGLLF | | |
| | SPAPQPRRLLPGPQENSVQSS | | |
| | ACGRQHLEPTGTIDMRGPGQ | | |
| | ADCAVAIGRPLGEVVTLRVL | | |
| | ESSLNCSAGDMLLLWGRLT | | |
| | WRKMCRKLLDMTFSSKTNT | | |
| | LVVRQRCGRPGGGVLLRYG | | |
| | SQLAPETFYRECDMQLFGP | | |
| | WGEIVSPSLSPATSNAGGCR | | |
| | LFINVAPHARIAIHALATNM | | |
| | GAGTEGANASYILIRDTHSL | | |
| | RTTAFHGQQVLYWESESSQ | | |
| | AEMEFSEGFLKAQASLRGQ | | |
| | YWTLQSWVPEMQDPQSWK | | |
| | GKEGT | | |
| FOXP3 | MPNPRPGKPSAPSLALGPSP GASPSWRAAPKASDLLGAR GPGGTFQGRDLRGGAHASSS SLNPMPPSQLQLPTLPLVMV APSGARLGPLPHLQALLQDR PHFMHQLSTVDAHARTPVL QVHPLESPAMISLTPPTTATG VFSLKARPGLPPGINVASLE WVSREPALLCTFPNPSAPRK DSTLSAVPQSSYPLLANGVC | Immune cells | 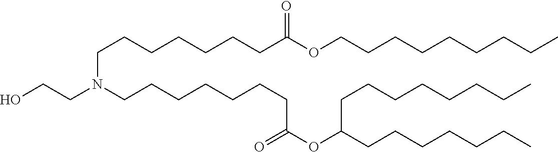 [SMILES: CCCCCCCCCOC(=O)CCCCCCCN(CCO)CCCCCCCC(=O)OC(CCCCCCC)CCCCCCCC] |
| | KWPGCEKVFEEPEDFLKHC | | (50 mol %) |
| | QADHLLDEKGRAQCLLQRE | | DSPC (10 mol %) |
| | MVQSLEQQLVLEKEKLSAM | | Beta-sitosterol (28.5% mol %) |
| | QAHLAGKMALTKASSVASS | | Cholesterol (10 mol %) |
| | DKGSCCIVAAGSQGPVVPA | | PEG DMG (1.5 mol %) |
| | WSGPREAPDSLFAVRRHLW | | |
| | GSHGNSTFPEFLHNMDYFKF | | |
| | HNMRPPFTYATLIRWAILEA | | |
| | PEKQRTLNEIYHWFTRMFAF | | |
| | FRNHPATWKNAIRHNLSLH | | |
| | KCFVRVESEKGAVWTVDEL | | |
| | EFRKKRSQRPSRCSNPTPGP | | |
| IL-10 | SPGQGTQSENSCTHFPGNLP NMLRDLRDAFSRVKTFFQM KDQLDNLLLKESLLEDFKGY LGCQALSEMIQFYLEEVMPQ AENQDPDIKAHVNSLGENLK TLRLRLRRCHRFLPCENKSK AVEQVKNAFNKLQEKGIYK AMSEFDIFINYIEAYMTMKIR N | Immune cells | 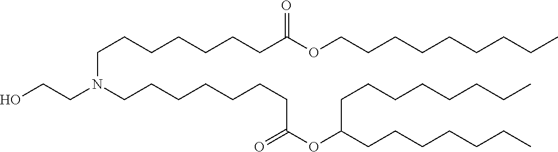 [SMILES: CCCCCCCCCOC(=O)CCCCCCCN(CCO)CCCCCCCC(=O)OC(CCCCCCC)CCCCCCCC] |
| | | | (50 mol %) |
| | | | DSPC (10 mol %) |
| | | | Beta-sitosterol (28.5% mol %) |
| | | | Cholesterol (10 mol %) |
| | | | PEG DMG (1.5 mol %) |
| IL-2 | APTSSSTKKTQLQLEHLLLD LQMILNGINNYKNPKLTRML TFKFYMPKKATELKHLQCLE EELKPLEEVLNLAQSKNFHL RPRDLISNINVIVLELKGSET TFMCEYADETATIVEFLNRW ITFCQSIISTLT | Immune cells | 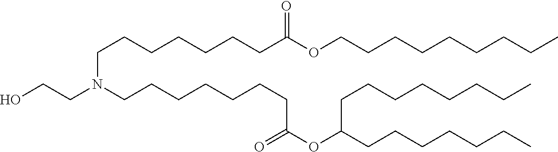 [SMILES: CCCCCCCCCOC(=O)CCCCCCCN(CCO)CCCCCCCC(=O)OC(CCCCCCC)CCCCCCCC] |
| | | | (50 mol %) |
| | | | DSPC (10 mol %) |
| | | | Beta-sitosterol (28.5% mol %) |
| | | | Cholesterol (10 mol %) |
| | | | PEG DMG (1.5 mol %) |

In some embodiments, the expression sequence encodes a therapeutic protein. In some embodiments, the expression sequence encodes a cytokine, e.g., IL-12p70, IL-15, IL-2, IL-18, IL-21, IFN-α, IFN-β, IL-10, TGF-beta, IL-4, or IL-35, or a functional fragment thereof. In some embodiments, the expression sequence encodes an immune checkpoint inhibitor. In some embodiments, the expression sequence encodes an agonist (e.g., a TNFR family member such as CD137L, OX40L, ICOSL, LIGHT, or CD70). In some embodiments, the expression sequence encodes a chimeric antigen receptor. In some embodiments, the expression sequence encodes an inhibitory receptor agonist (e.g., PDL1, PDL2, Galectin-9, VISTA, B7H4, or MHCII) or inhibitory receptor (e.g., PD1, CTLA4, TIGIT, LAG3, or TIM3). In some embodiments, the expression sequence encodes an inhibitory receptor antagonist. In some embodiments, the expression sequence encodes one or more TCR chains (alpha and beta chains or gamma and delta chains). In some embodiments, the expression sequence encodes a secreted T cell or immune cell engager (e.g., a bispecific antibody such as BiTE, targeting, e.g., CD3, CD137, or CD28 and a tumor-expressed protein e.g., CD19, CD20, or BCMA etc.). In some embodiments, the expression sequence encodes a transcription factor (e.g., FOXP3, HELIOS, TOX1, or TOX2). In some embodiments, the expression sequence encodes an immunosuppressive enzyme (e.g., IDO or CD39/CD73). In some embodiments, the expression sequence encodes a GvHD (e.g., anti-HLA-A2 CAR-Tregs).

In some embodiments, a polynucleotide encodes a protein that is made up of subunits that are encoded by more than one gene. For example, the protein may be a heterodimer, wherein each chain or subunit of the protein is encoded by a separate gene. It is possible that more than one circRNA molecule is delivered in the transfer vehicle and each circRNA encodes a separate subunit of the protein. Alternatively, a single circRNA may be engineered to encode more than one subunit. In certain embodiments, separate circRNA molecules encoding the individual subunits may be administered in separate transfer vehicles.

### 3.1 Cytokines

Descriptions and/or amino acid sequences of IL-2, IL-7, IL-10, IL-12, IL-15, IL-18, IL-27beta, IFNgamma, and/or TGFbeta1 are provided herein and at the www.uniprot.org database at accession numbers: P60568 (IL-2), P29459 (IL-12A), P29460 (IL-12B), P13232 (IL-7), P22301 (IL-10), P40933 (IL-15), Q14116 (IL-18), Q14213 (IL-27beta), P01579 (IFNgamma), and/or P01137 (TGFbeta1).

### 3.2 PD-1 and PD-L1 Antagonists

In some embodiments, a PD-1 inhibitor is pembrolizumab, pidilizumab, or nivolumab. In some embodiments, Nivolumab is described in WO2006/121168. In some embodiments, Pembrolizumab is described in WO2009/114335. In some embodiments, Pidilizumab is described in WO2009/101611. Additional anti-PD1 antibodies are described in U.S. Pat. No. 8,609,089, US 2010028330, US 20120114649, WO2010/027827 and WO2011/066342.

In some embodiments, a PD-L1 inhibitor is atezolizumab, avelumab, durvalumab, BMS-936559, or CK-301.

Descriptions and/or amino acid sequences of heavy and light chains of PD-1, and/or PD-L1 antibodies are provided herein and at the www.drugbank.ca database at accession numbers: DB09037 (Pembrolizumab), DB09035 (Nivolumab), DB15383 (Pidilizumab), DB11595 (Atezolizumab), DB11945 (Avelumab), and DB11714 (Durvalumab).

### 3.3 T Cell Receptors

TCRs are described using the International Immunogenetics (IMGT) TCR nomenclature, and links to the IMGT public database of TCR sequences. Native alpha-beta heterodimeric TCRs have an alpha chain and a beta chain. Broadly, each chain may comprise variable, joining and constant regions, and the beta chain also usually contains a short diversity region between the variable and joining regions, but this diversity region is often considered as part of the joining region. Each variable region may comprise three CDRs (Complementarity Determining Regions) embedded in a framework sequence, one being the hypervariable region named CDR3. There are several types of alpha chain variable (Vα) regions and several types of beta chain variable (Vβ) regions distinguished by their framework, CDR1 and CDR2 sequences, and by a partly defined CDR3 sequence. The Vα types are referred to in IMGT nomenclature by a unique TRAV number. Thus, “TRAV21” defines a TCR Vα region having unique framework and CDR1 and CDR2 sequences, and a CDR3 sequence which is partly defined by an amino acid sequence which is preserved from TCR to TCR but which also includes an amino acid sequence which varies from TCR to TCR. In the same way, “TRBV5-1” defines a TCR Vβ region having unique framework and CDR1 and CDR2 sequences, but with only a partly defined CDR3 sequence.

The joining regions of the TCR are similarly defined by the unique IMGT TRAJ and TRBJ nomenclature, and the constant regions by the IMGT TRAC and TRBC nomenclature.

The beta chain diversity region is referred to in IMGT nomenclature by the abbreviation TRBD, and, as mentioned, the concatenated TRBD/TRBJ regions are often considered together as the joining region.

The unique sequences defined by the IMGT nomenclature are widely known and accessible to those working in the TCR field. For example, they can be found in the IMGT public database. The “T cell Receptor Factsbook”, (2001) LeFranc and LeFranc, Academic Press, ISBN 0-12-441352-8 also discloses sequences defined by the IMGT nomenclature, but because of its publication date and consequent time-lag, the information therein sometimes needs to be confirmed by reference to the IMGT database.

Native TCRs exist in heterodimeric αβ or γδ forms. However, recombinant TCRs consisting of αα or ββ homodimers have previously been shown to bind to peptide MHC molecules. Therefore, the TCR of the invention may be a heterodimeric αβ TCR or may be an αα or ββ homodimeric TCR.

For use in adoptive therapy, an αβ heterodimeric TCR may, for example, be transfected as full length chains having both cytoplasmic and transmembrane domains. In certain embodiments TCRs of the invention may have an introduced disulfide bond between residues of the respective constant domains, as described, for example, in WO 2006/000830.

TCRs of the invention, particularly alpha-beta heterodimeric TCRs, may comprise an alpha chain TRAC constant domain sequence and/or a beta chain TRBC1 or TRBC2 constant domain sequence. The alpha and beta chain constant domain sequences may be modified by truncation or substitution to delete the native disulfide bond between Cys4 of exon 2 of TRAC and Cys2 of exon 2 of TRBC1 or TRBC2. The alpha and/or beta chain constant domain sequence(s) may also be modified by substitution of cysteine residues for Thr 48 of TRAC and Ser 57 of TRBC1 or TRBC2, the said cysteines forming a disulfide bond between the alpha and beta constant domains of the TCR.

Binding affinity (inversely proportional to the equilibrium constant K_{D}) and binding half-life (expressed as T½) can be determined by any appropriate method. It will be appreciated that doubling the affinity of a TCR results in halving the K_{D}. T½ is calculated as ln 2 divided by the off-rate (koff). So doubling of T½ results in a halving in koff. K_{D} and koff values for TCRs are usually measured for soluble forms of the TCR, i.e. those forms which are truncated to remove cytoplasmic and transmembrane domain residues. Therefore, it is to be understood that a given TCR has an improved binding affinity for, and/or a binding half-life for the parental TCR if a soluble form of that TCR has the said characteristics. Preferably the binding affinity or binding half-life of a given TCR is measured several times, for example 3 or more times, using the same assay protocol, and an average of the results is taken.

Since the TCRs of the invention have utility in adoptive therapy, the invention includes a non-naturally occurring and/or purified and/or engineered cell, especially a T-cell, presenting a TCR of the invention. There are a number of methods suitable for the transfection of T cells with nucleic acid (such as DNA, cDNA or RNA) encoding the TCRs of the invention (see for example Robbins et al., (2008) J Immunol. 180: 6116-6131). T cells expressing the TCRs of the invention will be suitable for use in adoptive therapy-based treatment of cancers such as those of the pancreas and liver. As will be known to those skilled in the art, there are a number of suitable methods by which adoptive therapy can be carried out (see for example Rosenberg et al., (2008) Nat Rev Cancer 8(4): 299-308).

As is well-known in the art, TCRs of the invention may be subject to post-translational modifications when expressed by transfected cells. Glycosylation is one such modification, which may comprise the covalent attachment of oligosaccharide moieties to defined amino acids in the TCR chain. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (i.e. oligosaccharide type, covalent linkage and total number of attachments) can influence protein function. Therefore, when producing recombinant proteins, controlling glycosylation is often desirable. Glycosylation of transfected TCRs may be controlled by mutations of the transfected gene (Kuball J et al. (2009), J Exp Med 206(2):463-475). Such mutations are also encompassed in this invention.

A TCR may be specific for an antigen in the group MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (AGE-B4), tyrosinase, brain glycogen phosphorylase, Melan-A, MAGE-C1, MAGE-C2, NY-ESO-1, LAGE-1, SSX-1, SSX-2(HOM-MEL-40), SSX-1, SSX-4, SSX-5, SCP-1, CT-7, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RARa fusion protein, PTPRK, K-ras, N-ras, Triosephosphate isomerase, GnTV, Herv-K-mel, Lage-1, Mage-C2, NA-88, Lage-2, SP17, and TRP2-Int2, (MART-I), gp100 (Pmel 17), TRP-1, TRP-2, MAGE-1, MAGE-3, p15(58), CEA, NY-ESO (LAGE), SCP-1, Hom/Mel-40, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, .beta.-Catenin, CDK4, Mum-1, p16, TAGE, PSMA, PSCA, CT7, telomerase, 43-9F, 5T4, 791Tgp72, α-fetoprotein (AFP), 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, and TPS.

### 3.4 Transcription Factors

Regulatory T cells (Treg) are important in maintaining homeostasis, controlling the magnitude and duration of the inflammatory response, and in preventing autoimmune and allergic responses.

In general, Tregs are thought to be mainly involved in suppressing immune responses, functioning in part as a “self-check” for the immune system to prevent excessive reactions. In particular, Tregs are involved in maintaining tolerance to self-antigens, harmless agents such as pollen or food, and abrogating autoimmune disease.

Tregs are found throughout the body including, without limitation, the gut, skin, lung, and liver. Additionally, Treg cells may also be found in certain compartments of the body that are not directly exposed to the external environment such as the spleen, lymph nodes, and even adipose tissue. Each of these Treg cell populations is known or suspected to have one or more unique features and additional information may be found in Lehtimaki and Lahesmaa, Regulatory T cells control immune responses through their non-redundant tissue specific features, 2013, FRONTIERS IN IMMUNOL., 4(294): 1-10, the disclosure of which is hereby incorporated in its entirety.

Typically, Tregs are known to require TGF-β and IL-2 for proper activation and development. Tregs, expressing abundant amounts of the IL-2 receptor (IL-2R), are reliant on IL-2 produced by activated T cells. Tregs are known to produce both IL-10 and TGF-β, both potent immunosuppressive cytokines. Additionally, Tregs are known to inhibit the ability of antigen presenting cells (APCs) to stimulate T cells. One proposed mechanism for APC inhibition is via CTLA-4, which is expressed by Foxp3+ Treg. It is thought that CTLA-4 may bind to B7 molecules on APCs and either block these molecules or remove them by causing internalization resulting in reduced availability of B7 and an inability to provide adequate co-stimulation for immune responses. Additional discussion regarding the origin, differentiation and function of Treg may be found in Dhamne et al., Peripheral and thymic Foxp3+ regulatory T cells in search of origin, distinction, and function, 2013, Frontiers in Immunol., 4 (253): 1-11, the disclosure of which is hereby incorporated in its entirety.

Descriptions and/or amino acid sequences of FOXP3, STAT5B, and/or HELIOS are provided herein and at the www.uniprot.org database at accession numbers: Q9BZS1 (FOXP3), P51692 (STAT5b), and/or Q9UKS7 (HELIOS).

### Foxp3

In some embodiments, a transcription factor is the Forkhead box P3 transcription factor (Foxp3). Foxp3 has been shown to be a key regulator in the differentiation and activity of Treg. In fact, loss-of-function mutations in the Foxp3 gene have been shown to lead to the lethal IPEX syndrome (immune dysregulation, polyendocrinopathy, enteropathy, X-linked). Patients with IPEX suffer from severe autoimmune responses, persistent eczema, and colitis. Regulatory T (Treg) cells expressing Foxp3 play a key role in limiting inflammatory responses in the intestine (Josefowicz, S. Z. et al. Nature, 2012, 482, 395-U1510).

### STAT

Members of the signal transducer and activator of transcription (STAT) protein family are intracellular transcription factors that mediate many aspects of cellular immunity, proliferation, apoptosis and differentiation. They are primarily activated by membrane receptor-associated Janus kinases (JAK). Dysregulation of this pathway is frequently observed in primary tumors and leads to increased angiogenesis, enhanced survival of tumors and immunosuppression. Gene knockout studies have provided evidence that STAT proteins are involved in the development and function of the immune system and play a role in maintaining immune tolerance and tumor surveillance.

There are seven mammalian STAT family members that have been identified: STAT1, STAT2, STAT3, STAT4, STAT5 (including STAT5A and STAT5B), and STATE.

Extracellular binding of cytokines or growth factors induce activation of receptor-associated Janus kinases, which phosphorylate a specific tyrosine residue within the STAT protein promoting dimerization via their SH2 domains. The phosphorylated dimer is then actively transported to the nucleus via an importin α/β ternary complex. Originally, STAT proteins were described as latent cytoplasmic transcription factors as phosphorylation was thought to be required for nuclear retention. However, unphosphorylated STAT proteins also shuttle between the cytosol and nucleus, and play a role in gene expression. Once STAT reaches the nucleus, it binds to a consensus DNA-recognition motif called gamma-activated sites (GAS) in the promoter region of cytokine-inducible genes and activates transcription. The STAT protein can be dephosphorylated by nuclear phosphatases, which leads to inactivation of STAT and subsequent transport out of the nucleus by a exportin-RanGTP complex.

In some embodiments, a STAT protein of the present disclosure may be a STAT protein that comprises a modification that modulates its expression level or activity. In some embodiments such modifications include, among other things, mutations that effect STAT dimerization, STAT protein binding to signaling partners, STAT protein localization or STAT protein degradation. In some embodiments, a STAT protein of the present disclosure is constitutively active. In some embodiments, a STAT protein of the present disclosure is constitutively active due to constitutive dimerization. In some embodiments, a STAT protein of the present disclosure is constitutively active due to constitutive phosphorylation as described in Onishi, M. et al., Mol. Cell. Biol. July 1998 vol. 18 no. 7 3871-3879 the entirety of which is herein incorporated by reference.

### 3.5 Chimeric Antigen Receptors

Chimeric antigen receptors (CARs or CAR-Ts) are genetically-engineered receptors. These engineered receptors may be inserted into and expressed by immune cells, including T cells via circular RNA as described herein. With a CAR, a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell. In some embodiments, the CAR encoded by the polynucleotide comprises (i) an antigen-binding molecule that specifically binds to a target antigen, (ii) a hinge domain, a transmembrane domain, and an intracellular domain, and (iii) an activating domain.

In some embodiments, an orientation of the CARs in accordance with the disclosure comprises an antigen binding domain (such as an scFv) in tandem with a costimulatory domain and an activating domain. The costimulatory domain may comprise one or more of an extracellular portion, a transmembrane portion, and an intracellular portion. In other embodiments, multiple costimulatory domains may be utilized in tandem.

### Antigen Binding Domain

CARs may be engineered to bind to an antigen (such as a cell-surface antigen) by incorporating an antigen binding molecule that interacts with that targeted antigen. In some embodiments, the antigen binding molecule is an antibody fragment thereof, e.g., one or more single chain antibody fragment (scFv). An scFv is a single chain antibody fragment having the variable regions of the heavy and light chains of an antibody linked together. See U.S. Pat. Nos. 7,741,465, and 6,319,494 as well as Eshhar et al., Cancer Immunol Immunotherapy (1997) 45: 131-136. An scFv retains the parent antibody's ability to specifically interact with target antigen. scFvs are useful in chimeric antigen receptors because they may be engineered to be expressed as part of a single chain along with the other CAR components. Id. See also Krause et al., J. Exp. Med., Volume 188, No. 4, 1998 (619-626); Finney et al., Journal of Immunology, 1998, 161: 2791-2797. It will be appreciated that the antigen binding molecule is typically contained within the extracellular portion of the CAR such that it is capable of recognizing and binding to the antigen of interest. Bispecific and multispecific CARs are contemplated within the scope of the invention, with specificity to more than one target of interest.

In some embodiments, the antigen binding molecule comprises a single chain, wherein the heavy chain variable region and the light chain variable region are connected by a linker. In some embodiments, the VH is located at the N terminus of the linker and the VL is located at the C terminus of the linker. In other embodiments, the VL is located at the N terminus of the linker and the VH is located at the C terminus of the linker. In some embodiments, the linker comprises at least about 5, at least about 8, at least about 10, at least about 13, at least about 15, at least about 18, at least about 20, at least about 25, at least about 30, at least about 35, at least about 40, at least about 45, at least about 50, at least about 60, at least about 70, at least about 80, at least about 90, or at least about 100 amino acids.

In some embodiments, the antigen binding molecule comprises a nanobody. In some embodiments, the antigen binding molecule comprises a DARPin. In some embodiments, the antigen binding molecule comprises an anticalin or other synthetic protein capable of specific binding to target protein.

In some embodiments, the CAR comprises an antigen binding domain specific for an antigen selected from the group CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GaINAca-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, HER2, HER3, Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WT1), Cancer/testis antigen 1 (NY-ESO-1), Cancer/testis antigen 2 (LAGE-1a), MAGE family members (including MAGE-A1, MAGE-A3 and MAGE-A4), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, surviving, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), MUC16, 5T4, 8H9, αvβθ integrin, αvβ6 integrin, alphafetoprotein (AFP), B7-H6, ca-125, CA9, CD44, CD44v7/8, CD52, E-cadherin, EMA (epithelial membrane antigen), epithelial glycoprotein-2 (EGP-2), epithelial glycoprotein-40 (EGP-40), ErbB4, epithelial tumor antigen (ETA), folate binding protein (FBP), kinase insert domain receptor (KDR), k-light chain, L1 cell adhesion molecule, MUC18, NKG2D, oncofetal antigen (h5T4), tumor/testis-antigen 1B, GAGE, GAGE-1, BAGE, SCP-1, CTZ9, SAGE, CAGE, CT10, MART-1, immunoglobulin lambda-like polypeptide 1 (IGLL1), Hepatitis B Surface Antigen Binding Protein (HBsAg), viral capsid antigen (VCA), early antigen (EA), EBV nuclear antigen (EBNA), HHV-6 p41 early antigen, HHV-6B U94 latent antigen, HHV-6B p98 late antigen, cytomegalovirus (CMV) antigen, large T antigen, small T antigen, adenovirus antigen, respiratory syncytial virus (RSV) antigen, haemagluttinin (HA), neuraminidase (NA), parainfluenza type 1 antigen, parainfluenza type 2 antigen, parainfluenza type 3 antigen, parainfluenza type 4 antigen, Human Metapneumovirus (HMPV) antigen, hepatitis C virus (HCV) core antigen, HIV p24 antigen, human T-cell lymphotrophic virus (HTLV-1) antigen, Merkel cell polyoma virus small T antigen, Merkel cell polyoma virus large T antigen, Kaposi sarcoma-associated herpesvirus (KSHV) lytic nuclear antigen and KSHV latent nuclear antigen. In some embodiments, an antigen binding domain comprises SEQ ID NO: 321 and/or 322.

### Hinge/Spacer Domain

In some embodiments, a CAR of the instant disclosure comprises a hinge or spacer domain. In some embodiments, the hinge/spacer domain may comprise a truncated hinge/spacer domain (THD) the THD domain is a truncated version of a complete hinge/spacer domain (“CHD”). In some embodiments, an extracellular domain is from or derived from (e.g., comprises all or a fragment of) ErbB2, glycophorin A (GpA), CD2, CD3 delta, CD3 epsilon, CD3 gamma, CD4, CD7, CD8a, CD8[T CD11a (IT GAL), CD11b (IT GAM), CD11c (ITGAX), CD11d (IT GAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (0X40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRT AM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MEW class 1 molecule, MHC class 2 molecule, a TNF receptor protein, an immunoglobulin protein, a cytokine receptor, an integrin, activating NK cell receptors, a Toll ligand receptor, and fragments or combinations thereof. A hinge or spacer domain may be derived either from a natural or from a synthetic source.

In some embodiments, a hinge or spacer domain is positioned between an antigen binding molecule (e.g., an scFv) and a transmembrane domain. In this orientation, the hinge/spacer domain provides distance between the antigen binding molecule and the surface of a cell membrane on which the CAR is expressed. In some embodiments, a hinge or spacer domain is from or derived from an immunoglobulin. In some embodiments, a hinge or spacer domain is selected from the hinge/spacer regions of IgG1, IgG2, IgG3, IgG4, IgA, IgD, IgE, IgM, or a fragment thereof. In some embodiments, a hinge or spacer domain comprises, is from, or is derived from the hinge/spacer region of CD8 alpha. In some embodiments, a hinge or spacer domain comprises, is from, or is derived from the hinge/spacer region of CD28. In some embodiments, a hinge or spacer domain comprises a fragment of the hinge/spacer region of CD8 alpha or a fragment of the hinge/spacer region of CD28, wherein the fragment is anything less than the whole hinge/spacer region. In some embodiments, the fragment of the CD8 alpha hinge/spacer region or the fragment of the CD28 hinge/spacer region comprises an amino acid sequence that excludes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids at the N-terminus or C-Terminus, or both, of the CD8 alpha hinge/spacer region, or of the CD28 hinge/spacer region.

### Transmembrane Domain

The CAR of the present disclosure may further comprise a transmembrane domain and/or an intracellular signaling domain. The transmembrane domain may be designed to be fused to the extracellular domain of the CAR. It may similarly be fused to the intracellular domain of the CAR. In some embodiments, the transmembrane domain that naturally is associated with one of the domains in a CAR is used. In some instances, the transmembrane domain may be selected or modified (e.g., by an amino acid substitution) to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein.

Transmembrane regions may be derived from (i.e. comprise) a receptor tyrosine kinase (e.g., ErbB2), glycophorin A (GpA), 4-1BB/CD137, activating NK cell receptors, an immunoglobulin protein, B7-H3, BAFFR, BFAME (SEAMF8), BTEA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (EIGHTR), IA4, ICAM-1, ICAM-1, Ig alpha (CD79a), IE-2R beta, IE-2R gamma, IE-7R alpha, inducible T cell costimulator (ICOS), integrins, ITGA4, ITGA4, ITGA6, IT GAD, ITGAE, ITGAE, IT GAM, ITGAX, ITGB2, ITGB7, ITGB1, KIRDS2, EAT, LFA-1, LFA-1, a ligand that specifically binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

In some embodiments, suitable intracellular signaling domain include, but are not limited to, activating Macrophage/Myeloid cell receptors CSFR1, MYD88, CD14, TIE2, TLR4, CR3, CD64, TREM2, DAP10, DAP12, CD169, DECTIN1, CD206, CD47, CD163, CD36, MARCO, TIM4, MERTK, F4/80, CD91, C1QR, LOX-1, CD68, SRA, BAI-1, ABCA7, CD36, CD31, Lactoferrin, or a fragment, truncation, or combination thereof.

In some embodiments, a receptor tyrosine kinase may be derived from (e.g., comprise) Insulin receptor (InsR), Insulin-like growth factor I receptor (IGF1R), Insulin receptor-related receptor (IRR), platelet derived growth factor receptor alpha (PDGFRa), platelet derived growth factor receptor beta (PDGFRfi). KIT proto-oncogene receptor tyrosine kinase (Kit), colony stimulating factor 1 receptor (CSFR), fms related tyrosine kinase 3 (FLT3), fms related tyrosine kinase 1 (VEGFR-1), kinase insert domain receptor (VEGFR-2), fms related tyrosine kinase 4 (VEGFR-3), fibroblast growth factor receptor 1 (FGFR1), fibroblast growth factor receptor 2 (FGFR2), fibroblast growth factor receptor 3 (FGFR3), fibroblast growth factor receptor 4 (FGFR4), protein tyrosine kinase 7 (CCK4), neurotrophic receptor tyrosine kinase 1 (trkA), neurotrophic receptor tyrosine kinase 2 (trkB), neurotrophic receptor tyrosine kinase 3 (trkC), receptor tyrosine kinase like orphan receptor 1 (ROR1), receptor tyrosine kinase like orphan receptor 2 (ROR2), muscle associated receptor tyrosine kinase (MuSK), MET proto-oncogene, receptor tyrosine kinase (MET), macrophage stimulating 1 receptor (Ron), AXL receptor tyrosine kinase (Axl), TYR03 protein tyrosine kinase (Tyro3), MER proto-oncogene, tyrosine kinase (Mer), tyrosine kinase with immunoglobulin like and EGF like domains 1 (TIE1), TEK receptor tyrosine kinase (TIE2), EPH receptor A1 (EphA1), EPH receptor A2 (EphA2), (EPH receptor A3) EphA3, EPH receptor A4 (EphA4), EPH receptor A5 (EphA5), EPH receptor A6 (EphA6), EPH receptor A7 (EphA7), EPH receptor A8 (EphA8), EPH receptor A10 (EphA10), EPH receptor B1 (EphB1), EPH receptor B2 (EphB2), EPH receptor B3 (EphB3), EPH receptor B4 (EphB4), EPH receptor B6 (EphB6), ret proto oncogene (Ret), receptor-like tyrosine kinase (RYK), discoidin domain receptor tyrosine kinase 1 (DDR1), discoidin domain receptor tyrosine kinase 2 (DDR2), c-ros oncogene 1, receptor tyrosine kinase (ROS), apoptosis associated tyrosine kinase (Lmr1), lemur tyrosine kinase 2 (Lmr2), lemur tyrosine kinase 3 (Lmr3), leukocyte receptor tyrosine kinase (LTK), ALK receptor tyrosine kinase (ALK), or serine/threonine/tyrosine kinase 1 (STYK1).

### Costimulatory Domain

In certain embodiments, the CAR comprises a costimulatory domain. In some embodiments, the costimulatory domain comprises 4-1BB (CD137), CD28, or both, and/or an intracellular T cell signaling domain. In a preferred embodiment, the costimulatory domain is human CD28, human 4-1BB, or both, and the intracellular T cell signaling domain is human CD3 zeta (ζ). 4-1BB, CD28, CD3 zeta may comprise less than the whole 4-1BB, CD28 or CD3 zeta, respectively. Chimeric antigen receptors may incorporate costimulatory (signaling) domains to increase their potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Amur. Rev. Pharmacol. Toxicol. 56:59-83 (2016).

In some embodiments, a costimulatory domain comprises the amino acid sequence of SEQ ID NO: 318 or 320.

### Intracellular Signaling Domain

The intracellular (signaling) domain of the engineered T cells disclosed herein may provide signaling to an activating domain, which then activates at least one of the normal effector functions of the immune cell. Effector function of a T cell, for example, may be cytolytic activity or helper activity including the secretion of cytokines.

In some embodiments, suitable intracellular signaling domain include (e.g., comprise), but are not limited to 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD 19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD1a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, LFA-1, ligand that specifically binds with CD83, LIGHT, LTBR, Ly9 (CD229), Ly108, lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

CD3 is an element of the T cell receptor on native T cells, and has been shown to be an important intracellular activating element in CARs. In some embodiments, the CD3 is CD3 zeta. In some embodiments, the activating domain comprises an amino acid sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to the polypeptide sequence of SEQ ID NO: 319.

### 3.6 Trispecific Antigen-Binding Proteins and Bispecific Antigen-Binding Proteins

Disclosed herein are circular RNA polypeptides encoding trispecific antigen-binding proteins (TRITEs), bispecific antigen-binding proteins (BITEs), functional fragments thereof, and pharmaceutical compositions thereof. Recombinant expression vectors useful for making circular RNA encoding trispecific antigen-binding proteins or bispecific antigen binding proteins, and cells comprising the inventive circular RNA are also provided herein. Also provided are methods of using the disclosed trispecific antigen-binding proteins or the bispecific antigen-binding proteins in the prevention and/or treatment of liver diseases, conditions and disorders. The trispecific antigen-binding proteins are capable of specifically binding to a target antigen, e.g., a cancer antigen, as well as CD3, TCR, CD16A, or NKp46, and a liver retention domain or a half-life extension domain, such as a domain binding human serum albumin (HSA). In some embodiments, the TRITE or BITE is created within a patient's liver post-administration of a composition comprising the inventive circular RNA polypeptides to a patient in need thereof.

In one aspect, trispecific antigen-binding proteins comprise a domain (A) which specifically binds to CD3, TCR, CD16A, or NKp46, a domain (B) which specifically binds to a half-life extension molecule or a liver retention molecule, and a domain (C) which specifically binds to a target antigen, e.g., a cancer cell antigen. The three domains in trispecific antigen-binding proteins may be arranged in any order. Thus, it is contemplated that the domain order of the trispecific antigen-binding proteins are in any of the following orders: (A)-(B)-(C), (A)-(C)-(B), (B)-(A)-(C), (B)-(C)-(A), (C)-(B)-(A), or (C)-(A)-(B).

In some embodiments, the trispecific antigen-binding proteins have a domain order of (A)-(B)-(C). In some embodiments, the trispecific antigen-binding proteins have a domain order of (A)-(C)-(B). In some embodiments, the trispecific antigen binding proteins have a domain order of (B)-(A)-(C). In some embodiments, the trispecific antigen-binding proteins have a domain order of (B)-(C)-(A). In some embodiments, the trispecific antigen-binding proteins have a domain order of (C)-(B)-(A). In some embodiments, the trispecific antigen-binding proteins have a domain order of (C)-(A)-(B).

In an embodiment, a bispecific antigen-binding protein comprises a domain (A) which specifically binds to CD3, TCR, CD16A, or NKp46, and a domain (B) which specifically binds to a target antigen. The two domains in a bispecific antigen-binding protein are arranged in any order. Thus, it is contemplated that the domain order of the bispecific antigen-binding proteins may be: (A)-(B), or (B)-(A).

The trispecific antigen-binding proteins or bispecific antigen-binding proteins described herein are designed to allow specific targeting of cells expressing a target antigen by recruiting cytotoxic T cells or NK cells. This improves efficacy compared to ADCC (antibody dependent cell-mediated cytotoxicity), which uses full length antibodies directed to a sole antigen and is not capable of directly recruiting cytotoxic T cells. In contrast, by engaging CD3 molecules expressed specifically on these cells, the trispecific antigen-binding proteins or bispecific antigen-binding proteins can crosslink cytotoxic T cells or NK cells with cells expressing a target antigen in a highly specific fashion, thereby directing the cytotoxic potential of the recruited T cell or NK cell towards the target cell. The trispecific antigen-binding proteins or bispecific antigen-binding proteins described herein engage cytotoxic T cells via binding to the surface-expressed CD3 proteins, which form part of the TCR, or CD16A or NKp46, which activates NK cells. Simultaneous binding of several trispecific antigen-binding protein or bispecific antigen-binding proteins to CD3 and to a target antigen expressed on the surface of particular cells causes T cell activation and mediates the subsequent lysis of the particular target antigen expressing cell. Thus, trispecific antigen-binding or bispecific antigen-binding proteins are contemplated to display strong, specific and efficient target cell killing. In some embodiments, the trispecific antigen-binding proteins or bispecific antigen-binding proteins described herein stimulate target cell killing by cytotoxic T cells to eliminate pathogenic cells (e.g., tumor cells, virally or bacterially infected cells, autoreactive T cells, etc). In some embodiments, cells are eliminated selectively, thereby reducing the potential for toxic side effects. In some embodiments anti-41bb or CD137 binding domains are used as the t cell engager.

### Immune Cell Binding Domain

The specificity of the response of T cells is mediated by the recognition of antigen (displayed in context of a major histocompatibility complex, MHC) by the TCR. As part of the TCR, CD3 is a protein complex that includes a CD3γ (gamma) chain, a CD3δ (delta) chain, and two CD3ε (epsilon) chains which are present on the cell surface. CD3 associates with the α (alpha) and β (beta) chains of the TCR as well as CD3 ζ (zeta) altogether to comprise the complete TCR. Clustering of CD3 on T cells, such as by immobilized anti-CD3 antibodies leads to T cell activation similar to the engagement of the T cell receptor but independent of its clone-typical specificity.

In one aspect, the bispecific and trispecific proteins described herein comprise a domain which specifically binds to CD3. In one aspect, the trispecific proteins described herein comprise a domain which specifically binds to human CD3. In some embodiments, the trispecific proteins described herein comprise a domain which specifically binds to CD3γ. In some embodiments, the trispecific proteins described herein comprise a domain which specifically binds to CD36. In some embodiments, the trispecific proteins described herein comprise a domain which specifically binds to CD3ε.

In further embodiments, the trispecific proteins described herein comprise a domain which specifically binds to the TCR. In certain instances, the trispecific proteins described herein comprise a domain which specifically binds the α chain of the TCR. In certain instances, the trispecific proteins described herein comprise a domain which specifically binds the β chain of the TCR.

In some embodiments, a trispecific antigen binding protein or bispecific antigen binding protein comprises a NKp46 specific binder. In some embodiments, a trispecific antigen binding protein or bispecific antigen binding protein comprises a CD16A specific binder.

In some embodiments, the CD3, TCR, NKp46, or CD16A binding domain of the antigen-binding protein can be any domain that binds to CD3, TCR, NKp46, or CD16A including but not limited to domains from a monoclonal antibody, a polyclonal antibody, a recombinant antibody, a human antibody, a humanized antibody. In some instances, it is beneficial for the CD3, TCR, NKp46, or CD16A binding domain to be derived from the same species in which the trispecific antigen-binding protein will ultimately be used in. For example, for use in humans, it may be beneficial for the CD3, TCR, NKp46, or CD16A binding domain of the trispecific antigen-binding protein to comprise human or humanized residues from the antigen binding domain of an antibody or antibody fragment.

Thus, in one aspect, the antigen-binding domain comprises a humanized or human antibody or an antibody fragment, or a murine antibody or antibody fragment. In one embodiment, the humanized or human anti-CD3, TCR, NKp46, or CD16A binding domain comprises one or more (e.g., all three) light chain complementary determining region 1 (LC CDR1), light chain complementary determining region 2 (LC CDR2), and light chain complementary determining region 3 (LC CDR3) of a humanized or human anti-CD3, TCR, NKp46, or CD16A binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-CD3, TCR, NKp46, or CD16A binding domain described herein, e.g., a humanized or human anti-CD3, TCR, NKp46, or CD16A binding domain comprising one or more, e.g., all three, LC CDRs and one or more, e.g., all three, HC CDRs.

In some embodiments, the humanized or human anti-CD3, TCR, NKp46, or CD16A binding domain comprises a humanized or human heavy chain variable region specific to CD3, TCR, NKp46, or CD16A where the heavy chain variable region specific to CD3, TCR, NKp46, or CD16A comprises human or non-human heavy chain CDRs in a human heavy chain framework region.

In certain instances, the complementary determining regions of the heavy chain and/or the light chain are derived from known anti-CD3 antibodies, such as, for example, muromonab-CD3 (OKT3), otelixizumab (TRX4), teplizumab (MGA031), visilizumab (Nuvion), SP34, TR-66 or X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31.

In some embodiments, an anti-NKp46 binding domain comprises an antibody or fragment thereof described in U.S. patent application Ser. No. 16/451,051. In some embodiments, an anti-NKp46 binding domain comprises the antibodies BAB281, 9E2, 195314 or a fragment thereof.

In one embodiment, the anti-CD3, TCR, NKp46, or CD16A binding domain is a single chain variable fragment (scFv) comprising a light chain and a heavy chain of an amino acid sequence provided herein. In an embodiment, the anti-CD3, TCR, NKp46, or CD16A binding domain comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-CD3 binding domain is a scFv, and a light chain variable region comprising an amino acid sequence described herein, is attached to a heavy chain variable region comprising an amino acid sequence described herein, via a scFv linker. The light chain variable region and heavy chain variable region of a scFv can be, e.g., in any of the following orientations: light chain variable region-scFv linker-heavy chain variable region or heavy chain variable region-scFv linker-light chain variable region.

In some embodiments, CD3, TCR, NKp46, or CD16A binding domain of trispecific antigen-binding protein has an affinity to CD3, TCR, NKp46, or CD16A on CD3, TCR, NKp46, or CD16A expressing cells with a KD of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In some embodiments, the CD3 binding domain of MSLN trispecific antigen-binding protein has an affinity to CD3ε, γ, or δ with a KD of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In further embodiments, CD3, TCR, NKp46, or CD16A binding domain of trispecific antigen-binding protein has low affinity to CD3, TCR, NKp46, or CD16A, i.e., about 100 nM or greater.

The affinity to bind to CD3, TCR, NKp46, or CD16A can be determined, for example, by the ability of the trispecific antigen-binding protein itself or its CD3, TCR, NKp46, or CD16A binding domain to bind to CD3, TCR, NKp46, or CD16A coated on an assay plate; displayed on a microbial cell surface; in solution; etc. The binding activity of the trispecific antigen-binding protein itself or its CD3, TCR, NKp46, or CD16A binding domain of the present disclosure to CD3, TCR, NKp46, or CD16A can be assayed by immobilizing the ligand (e.g., CD3, TCR, NKp46, or CD16A) or the trispecific antigen-binding protein itself or its CD3, TCR, NKp46, or CD16A binding domain, to a bead, substrate, cell, etc. Agents can be added in an appropriate buffer and the binding partners incubated for a period of time at a given temperature. After washes to remove unbound material, the bound protein can be released with, for example, SDS, buffers with a high pH, and the like and analyzed, for example, by Surface Plasmon Resonance (SPR).

In some embodiments, a bispecific antigen binding protein or bispecific antigen binding protein comprises a TCR binding domain. In some embodiments, a TCR binding domain is a viral antigen or a fragment thereof. In some embodiments, a viral antigen is from the families: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., Ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunya viruses, phleboviruses and Nairo viruses); Arenaviridae (hemorrhagic fever viruses); Reoviridae (e.g., reoviruses, orbiviruses and rotaviruses); Bornaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g., African swine fever virus); and unclassified viruses (e.g., the agent of delta hepatitis (thought to be a defective satellite of hepatitis B virus), Hepatitis C; Norwalk and related viruses, and astroviruses).

### Linkers

In the trispecific proteins described herein, the domains are linked by internal linkers L1 and L2, where L1 links the first and second domain of the trispecific proteins and L2 links the second and third domains of the trispecific proteins. In some embodiments, linkers L1 and L2 have an optimized length and/or amino acid composition. In some embodiments, linkers L1 and L2 are the same length and amino acid composition. In other embodiments, L1 and L2 are different. In certain embodiments, internal linkers L1 and/or L2 consist of 0, 1, 2, 3, 4, 5, 6, 7, 8, 9, 10, 11 or 12 amino acid residues. Thus, in certain instances, the internal linkers consist of about 12 or less amino acid residues. In the case of 0 amino acid residues, the internal linker is a peptide bond. In certain embodiments, internal linkers L1 and/or L2 consist of 15, 20 or 25 amino acid residues. In some embodiments, these internal linkers consist of about 3 to about 15, for example 8, 9 or 10 contiguous amino acid residues. Regarding the amino acid composition of the internal linkers L1 and L2, peptides are selected with properties that confer flexibility to the trispecific proteins, do not interfere with the binding domains as well as resist cleavage from proteases. For example, glycine and serine residues generally provide protease resistance. Examples of internal linkers suitable for linking the domains in the trispecific proteins include but are not limited to (GS)n, (GGS)n, (GGGS)n, (GGSG)n, (GGSGG)n, (GGGGS)n, (GGGGG)n, or (GGG)n, wherein n is 1, 2, 3, 4, 5, 6, 7, 8, 9, or 10. In one embodiment, internal linker L1 and/or L2 is (GGGGS)4 or (GGGGS)₃.

### Half-Life Extension Domain

Contemplated herein are domains which extend the half-life of an antigen-binding domain. Such domains are contemplated to include but are not limited to Albumin binding domains, Fc domains, small molecules, and other half-life extension domains known in the art.

Human albumin (ALB) is the most abundant protein in plasma, present at about 50 mg/ml and has a half-life of around 20 days in humans. ALB serves to maintain plasma pH, contributes to colloidal blood pressure, functions as carrier of many metabolites and fatty acids, and serves as a major drug transport protein in plasma.

Noncovalent association with albumin extends the elimination half-time of short lived proteins.

In one aspect, the trispecific proteins described herein comprise a half-life extension domain, for example a domain which specifically binds to ALB. In some embodiments, the ALB binding domain of a trispecific antigen-binding protein can be any domain that binds to ALB including but not limited to domains from a monoclonal antibody, a polyclonal antibody, a recombinant antibody, a human antibody, a humanized antibody. In some embodiments, the ALB binding domain is a single chain variable fragments (scFv), single-domain antibody such as a heavy chain variable domain (VH), a light chain variable domain (VL) and a variable domain (VHH) of camelid derived single domain antibody, peptide, ligand or small molecule entity specific for HSA. In certain embodiments, the ALB binding domain is a single-domain antibody. In other embodiments, the HSA binding domain is a peptide. In further embodiments, the HSA binding domain is a small molecule. It is contemplated that the HSA binding domain of MSLN trispecific antigen-binding protein is fairly small and no more than 25 kD, no more than 20 kD, no more than 15 kD, or no more than 10 kD in some embodiments. In certain instances, the ALB binding is 5 kD or less if it is a peptide or small molecule entity.

The half-life extension domain of a trispecific antigen-binding protein provides for altered pharmacodynamics and pharmacokinetics of the trispecific antigen-binding protein itself. As above, the half-life extension domain extends the elimination half-time. The half-life extension domain also alters pharmacodynamic properties including alteration of tissue distribution, penetration, and diffusion of the trispecific antigen-binding protein. In some embodiments, the half-life extension domain provides for improved tissue (including tumor) targeting, tissue distribution, tissue penetration, diffusion within the tissue, and enhanced efficacy as compared with a protein without a half-life extension domain. In one embodiment, therapeutic methods effectively and efficiently utilize a reduced amount of the trispecific antigen-binding protein, resulting in reduced side effects, such as reduced non-tumor cell cytotoxicity.

Further, the binding affinity of the half-life extension domain can be selected so as to target a specific elimination half-time in a particular trispecific antigen-binding protein. Thus, in some embodiments, the half-life extension domain has a high binding affinity. In other embodiments, the half-life extension domain has a medium binding affinity. In yet other embodiments, the half-life extension domain has a low or marginal binding affinity. Exemplary binding affinities include KD concentrations at 10 nM or less (high), between 10 nM and 100 nM (medium), and greater than 100 nM (low). As above, binding affinities to ALB are determined by known methods such as Surface Plasmon Resonance (SPR).

### Liver Retention Domain

Contemplated herein are domains which allows for and promotes a higher retention of the trispecific antigen-binding protein within liver. The liver retention domain of the trispecific antigen-binding protein is directed to targeting a liver cell moiety. In an embodiment, a liver cell includes but is not limited to a hepatocyte, hepatic stellate cell, sinusoidal endothelial cell.

In an embodiment, a liver cell contains a receptor that binds to a liver targeting moiety. In an embodiment, the liver targeting moiety includes, but is not limited to lactose, cyanuric chloride, cellobiose, polylysine, polyarginine, Mannose-6-phosphate, PDGF, human serum albumin, galactoside, galactosamine, linoleic acid, Apoliopoprotein A-1, Acetyl CKNEKKNIERNNKLKQPP-amide, glycyrrhizin, lactobionic acid, Mannose-BSA, BSA, poly-ACO-HAS, KLGR peptide, hyaluronic acid, IFN-alpha, cRGD peptide, 6-phosphate-HSA, retinol, lactobiotin, galactoside, pullulan, soybean steryglucoside, asialoorosomucoid, glycyrrhetinic acid/glycyrrhizin, linoleic acid, AMD3100, cleavable hyaluronic acid-glycyrrhetinic acid, Hepatitis B virus pre-S1 derived lipoprotein, Apo-A1, or LDL. In an embodiment, the liver cell receptor includes but is not limited to galactose receptor, mannose receptor, scavenger receptor, low-density lipoprotein receptor, HARE, CD44, IFNα receptor, collagen type VI receptor, 6-phosphate/insulin-like growth factor 2 receptor, platelet-derived growth factor receptor β, RBP receptor, αVβ3 integrin receptor, ASGP receptor, glycyrrhetinic acid/glycyrrhizin receptor, PPAR, Heparan sulfate glycosaminoglycan receptor, CXC receptor type 4, glycyrrhetinic acid receptor, HBVP receptor, HDL receptor, scavenger receptor class B member 1 LDL receptor or combination thereof.

### Target Antigen Binding Domain

The trispecific antigen-binding proteins and bispecific antigen-binding proteins described herein comprise a domain that binds to a target antigen. A target antigen is involved in and/or associated with a disease, disorder or condition, e.g., cancer. In some embodiments, a target antigen is a tumor antigen. In some embodiments, the target antigen is NY-ESO-1, SSX-2, Sp 17, AFP, Glypican-3, Gpa33, Annexin-A2, WT1, PSMA, Midkine, PRAME, Survivin, MUC-1. P53, CEA, RAS, Hsp70, Hsp27, squamous cell carcinoma antigen (SCCA), GP73, TAG-72, or a protein in the MAGE family.

In some embodiments, a target antigen is one found on a non-liver tumor cell that has metastasized into the liver. In some embodiments, a bispecific antigen-binding protein or trispecific antigen binding protein comprises a target antigen binding domain specific for group CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GaINAca-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, HER2, HER3, Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), claudin 18.2 (CLDN18.2), thyroid stimulating hormone receptor (TSHR), G protein-coupled receptor class C group 5, member D (GPRC5D), chromosome X open reading frame 61 (CXORF61), CD97, or CD179a. In some embodiments, a target antigen is an antigen associated with a viral disease, e.g., a viral antigen. In some embodiments, a target antigen is a hepatitis A, hepatitis B, hepatitis C, hepatitis D or hepatitis E antigen.

The design of the trispecific antigen-binding proteins described herein allows the binding domain to a liver target antigen to be flexible in that the binding domain to a liver target antigen can be any type of binding domain, including but not limited to, domains from a monoclonal antibody, a polyclonal antibody, a recombinant antibody, a human antibody, a humanized antibody. In some embodiments, the binding domain to a liver target antigen is a single chain variable fragments (scFv), single-domain antibody such as a heavy chain variable domain (VH), a light chain variable domain (VL) and a variable domain (VHH) of camelid derived single domain antibody. In other embodiments, the binding domain to a liver target antigen is a non-Ig binding domain, i.e., antibody mimetic, such as anticalins, affilins, affibody molecules, affimers, affitins, alphabodies, avimers, DARPins, fynomers, kunitz domain peptides, and monobodies. In further embodiments, the binding domain to a liver target antigen is a ligand or peptide that binds to or associates with a target antigen.

### 3.7 PAH

In some embodiments, the present invention provides methods and compositions for delivering circRNA encoding PAH to a subject for the treatment of phenylketonuria (PKU). A suitable PAH circRNA encodes any full length, fragment or portion of a PAH protein which can be substituted for naturally-occurring PAH protein activity and/or reduce the intensity, severity, and/or frequency of one or more symptoms associated with PKU.

In some embodiments, a suitable RNA sequence for the present invention comprises a circRNA sequence encoding human PAH protein.

In some embodiments, a suitable RNA sequence may be an RNA sequence that encodes a homolog or an analog of human PAH. As used herein, a homolog or an analog of human PAH protein may be a modified human PAH protein containing one or more amino acid substitutions, deletions, and/or insertions as compared to a wild-type or naturally-occurring human PAH protein while retaining substantial PAH protein activity.

The present invention may be used to treat a subject who is suffering from or susceptible to Phenylketonuria (PKU). PKU is an autosomal recessive metabolic genetic disorder characterized by a mutation in the gene for the hepatic enzyme phenylalanine hydroxylase (PAH), rendering it nonfunctional. PAH is necessary to metabolize the amino acid phenylalanine (Phe) to the amino acid tyrosine (Tyr). When PAH activity is reduced, phenylalanine accumulates and is converted into phenylpyruvate (also known as phenylketone) which can be detected in the urine.

Phenylalanine is a large, neutral amino acid (LNAA). LNAAs compete for transport across the blood-brain barrier (BBB) via the large neutral amino acid transporter (LNAAT). Excess Phe in the blood saturates the transporter and tends to decrease the levels of other LNAAs in the brain. Because several of these other amino acids are necessary for protein and neurotransmitter synthesis, Phe buildup hinders the development of the brain, and can cause mental retardation.

In addition to hindered brain development, the disease can present clinically with a variety of symptoms including seizures, albinism hyperactivity, stunted growth, skin rashes (eczema), microcephaly, and/or a “musty” odor to the baby's sweat and urine, due to phenylacetate, one of the ketones produced). Untreated children are typically normal at birth, but have delayed mental and social skills, have a head size significantly below normal, and often demonstrate progressive impairment of cerebral function. As the child grows and develops, additional symptoms including hyperactivity, jerking movements of the arms or legs, EEG abnormalities, skin rashes, tremors, seizures, and severe learning disabilities tend to develop. However, PKU is commonly included in the routine newborn screening panel of most countries that is typically performed 2-7 days after birth.

If PKU is diagnosed early enough, an affected newborn can grow up with relatively normal brain development, but only by managing and controlling Phe levels through diet, or a combination of diet and medication. All PKU patients must adhere to a special diet low in Phe for optimal brain development. The diet requires severely restricting or eliminating foods high in Phe, such as meat, chicken, fish, eggs, nuts, cheese, legumes, milk and other dairy products. Starchy foods, such as potatoes, bread, pasta, and corn, must be monitored. Infants may still be breastfed to provide all of the benefits of breastmilk, but the quantity must also be monitored and supplementation for missing nutrients will be required. The sweetener aspartame, present in many diet foods and soft drinks, must also be avoided, as aspartame contains phenylalanine.

Throughout life, patients can use supplementary infant formulas, pills or specially formulated foods to acquire amino acids and other necessary nutrients that would otherwise be deficient in a low-phenylalanine diet. Some Phe is required for the synthesis of many proteins and is required for appropriate growth, but levels of it must be strictly controlled in PKU patients. Additionally, PKU patients must take supplements of tyrosine, which is normally derived from phenylalanine. Other supplements can include fish oil, to replace the long chain fatty acids missing from a standard Phe-free diet and improve neurological development and iron or carnitine. Another potential therapy for PKU is tetrahydrobiopterin (BH4), a cofactor for the oxidation of Phe that can reduce blood levels of Phe in certain patients. Patients who respond to BH4 therapy may also be able to increase the amount of natural protein that they can eat.

In some embodiments, the expression of PAH protein is detectable in liver, kidney, heart, spleen, serum, brain, skeletal muscle, lymph nodes, skin, and/or cerebrospinal fluid.

In some embodiments, administering the provided composition results in the expression of a PAH protein level at or above about 100 ng/mg, about 200 ng/mg, about 300 ng/mg, about 400 ng/mg, about 500 ng/mg, about 600 ng/mg, about 700 ng/mg, about 800 ng/mg, about 900 ng/mg, about 1000 ng/mg, about 1200 ng/mg or about 1400 ng/mg of total protein in the liver.

In some embodiments, the expression of the PAH protein is detectable 1 to 96 hours after administration. For example, in some embodiments, expression of PAH protein is detectable 1 to 84 hours, 1 to 72 hours, 1 to 60 hours, 1 to 48 hours, 1 to 36 hours, 1 to 24 hours, 1 to 12 hours, 1 to 10 hours, 1 to 8 hours, 1 to 6 hours, 1 to 4 hours, 1 to 2 hours, 2 to 96 hours, 2 to 84 hours, 2 to 72 hours, 2 to 60 hours, 2 to 48 hours, 2 to 36 hours, 2 to 24 hours, 2 to 12 hours, 2 to 10 hours, 2 to 8 hours, 2 to 6 hours, 2 to 4 hours, 4 to 96 hours, 4 to 84 hours, 4 to 72 hours, 4 to 60 hours, 4 to 48 hours, 4 to 36 hours, 4 to 24 hours, 4 to 12 hours, 4 to 10 hours, 4 to 8 hours, 4 to 6 hours, 6 to 96 hours, 6 to 84 hours, 6 to 72 hours, 6 to 60 hours, 6 to 48 hours, 6 to 36 hours, 6 to 24 hours, 6 to 12 hours, 6 to 10 hours, 6 to 8 hours, 8 to 96 hours, 8 to 84 hours, 8 to 72 hours, 8 to 60 hours, 8 to 48 hours, 8 to 36 hours, 8 to 24 hours, 8 to 12 hours, 8 to 10 hours, 10 to 96 hours, 10 to 84 hours, 10 to 72 hours, 10 to 60 hours, 10 to 48 hours, 10 to 36 hours, 10 to 24 hours, 10 to 12 hours, 12 to 96 hours, 12 to 84 hours, 12 to 72 hours, 12 to 60 hours, 12 to 48 hours, 12 to 36 hours, 12 to 24 hours, 24 to 96 hours, 24 to 84 hours, 24 to 72 hours, 24 to 60 hours, 24 to 48 hours, 24 to 36 hours, 36 to 96 hours, 36 to 84 hours, 36 to 72 hours, 36 to 60 hours, 36 to 48 hours, 48 to 96 hours, 48 to 84 hours, 48 to 72 hours, 48 to 60 hours, 48 to 84 hours, 48 to 72 hours, 48 to 60 hours, 60 to 96 hours, 60 to 84 hours, 60 to 72 hours, 72 hours to 96 hours, 72 hours to 84 hours, or 84 hours to 96 hours after administration. For example, in certain embodiments, the expression of the PAH protein is detectable 6, 12, 18, 24, 30, 36, 42, 48, 54, 60, 66, ₐnd/or 72 hours after the administration. In some embodiments, the expression of the PAH protein is detectable 1 day to 7 days after the administration. For example, in some embodiments, PAH protein is detectable 1 day, 2 days, 3 days, 4 days, 5 days, 6 days, and/or 7 days after the administration. In some embodiments, the expression of the PAH protein is detectable 1 week to 8 weeks after the administration. For example, in some embodiments, the expression of the PAH protein is detectable 1 week, 2 weeks, 3 weeks, and/or 4 weeks after the administration. In some embodiments, the expression of the PAH protein is detectable after a month after the administration.

### 3.8 CPS1

In some embodiments, the present invention provides methods and compositions for delivering circRNA encoding CPS1 to a subject for the treatment of CPS1 deficiency. A suitable CPS1 circRNA encodes any full length, fragment or portion of a CPS1 protein which can be substituted for naturally-occurring CPS1 protein activity and/or reduce the intensity, severity, and/or frequency of one or more symptoms associated with CPS1 deficiency.

In some embodiments, a suitable RNA sequence for the present invention comprises a circRNA sequence encoding human CPS1 protein.

In some embodiments, a suitable RNA sequence may be an RNA sequence that encodes a homolog or an analog of human CPS1. As used herein, a homolog or an analog of human CPS1 protein may be a modified human CPS1 protein containing one or more amino acid substitutions, deletions, and/or insertions as compared to a wild-type or naturally-occurring human CPS1 protein while retaining substantial CPS1 protein activity.

Carbamoyl phosphate synthetase I (CPS1) catalyzes the conversion of ammonia, bicarbonate and 2 ATP with formation of carbamoyl phosphate in the first step of the urea cycle. It also plays a role in the biosynthesis of arginine, which in turn is a substrate for the biosynthesis of NO, e.g. in the case of an endotoxin shock (c.f. Shoko Tabuchi et al., Regulation of Genes for Inducible Nitric Oxide Synthase and Urea Cycle Enzymes in Rat Liver in Endotoxin Shock, Biochemical and Biophysical Research Communications 268, 221-224 (2000)). CPS 1 should be distinguished from the cytosolic enzyme CPS 2, which likewise plays a role in the urea cycle but processes the substrate glutamine. It is known that CPS 1 is localized in mitochondria and occurs in this form in large amounts in liver tissue (it accounts for 2-6% of total liver protein). Its amino acid sequence and genetic localization have long been known (c.f. Haraguchi Y. et al., Cloning and sequence of a cDNA encoding human carbamyl phosphate synthetase I: molecular analysis of hyperammonemia, Gene 1991, Nov. 1; 107 (2); 335-340; cf. also the publication WO 03/089933 A1 of the Applicant). Regarding its physiological role, reference may be made to review articles such as, for example, H. M. Holder et al., Carbamoyl phosphate synthetase: an amazing biochemical odyssey from substrate to product, CMLS, Cell. Mol. Life Sci. 56 (1999) 507-522, and the literature referred to therein, and the introduction to the publication by Mikiko Ozaki et al., Enzyme-Linked Immunosorbent Assay of Carbamoylphosphate Synthetase I: Plasma Enzyme in Rat Experimental Hepatitis and Its Clearance, Enzyme Protein 1994, 95:48:213-221.

Carbamoyl phosphate synthetase I (CPS1) deficiency is a genetic disorder characterized by a mutation in the gene for the enzyme Carbamoyl phosphate synthetase I, affecting its ability to catalyze synthesis of carbamoyl phosphate from ammonia and bicarbonate. This reaction is the first step of the urea cycle, which is important in the removal of excess urea from cells. Defects in the CPS1 protein disrupt the urea cycle and prevent the liver from properly processing excess nitrogen into urea.

In some embodiments, administering the provided composition results in the expression of a CPS1 protein level at or above about 100 ng/mg, about 200 ng/mg, about 300 ng/mg, about 400 ng/mg, about 500 ng/mg, about 600 ng/mg, about 700 ng/mg, about 800 ng/mg, about 900 ng/mg, about 1000 ng/mg, about 1200 ng/mg or about 1400 ng/mg of total protein in the liver.

In some embodiments, the expression of the CPS1 protein is detectable 1 to 96 hours after administration. For example, in some embodiments, expression of CPS1 protein is detectable 1 to 84 hours, 1 to 72 hours, 1 to 60 hours, 1 to 48 hours, 1 to 36 hours, 1 to 24 hours, 1 to 12 hours, 1 to 10 hours, 1 to 8 hours, 1 to 6 hours, 1 to 4 hours, 1 to 2 hours, 2 to 96 hours, 2 to 84 hours, 2 to 72 hours, 2 to 60 hours, 2 to 48 hours, 2 to 36 hours, 2 to 24 hours, 2 to 12 hours, 2 to 10 hours, 2 to 8 hours, 2 to 6 hours, 2 to 4 hours, 4 to 96 hours, 4 to 84 hours, 4 to 72 hours, 4 to 60 hours, 4 to 48 hours, 4 to 36 hours, 4 to 24 hours, 4 to 12 hours, 4 to 10 hours, 4 to 8 hours, 4 to 6 hours, 6 to 96 hours, 6 to 84 hours, 6 to 72 hours, 6 to 60 hours, 6 to 48 hours, 6 to 36 hours, 6 to 24 hours, 6 to 12 hours, 6 to 10 hours, 6 to 8 hours, 8 to 96 hours, 8 to 84 hours, 8 to 72 hours, 8 to 60 hours, 8 to 48 hours, 8 to 36 hours, 8 to 24 hours, 8 to 12 hours, 8 to 10 hours, 10 to 96 hours, 10 to 84 hours, 10 to 72 hours, 10 to 60 hours, 10 to 48 hours, 10 to 36 hours, 10 to 24 hours, 10 to 12 hours, 12 to 96 hours, 12 to 84 hours, 12 to 72 hours, 12 to 60 hours, 12 to 48 hours, 12 to 36 hours, 12 to 24 hours, 24 to 96 hours, 24 to 84 hours, 24 to 72 hours, 24 to 60 hours, 24 to 48 hours, 24 to 36 hours, 36 to 96 hours, 36 to 84 hours, 36 to 72 hours, 36 to 60 hours, 36 to 48 hours, 48 to 96 hours, 48 to 84 hours, 48 to 72 hours, 48 to 60 hours, 48 to 84 hours, 48 to 72 hours, 48 to 60 hours, 60 to 96 hours, 60 to 84 hours, 60 to 72 hours, 72 hours to 96 hours, 72 hours to 84 hours, or 84 hours to 96 hours after administration. For example, in certain embodiments, the expression of the CPS1 protein is detectable 6, 12, 18, 24, 30, 36, 42, 48, 54, 60, 66, and/or 72 hours after the administration. In some embodiments, the expression of the CPS1 protein is detectable 1 day to 7 days after the administration. For example, in some embodiments, CPS1 protein is detectable 1 day, 2 days, 3 days, 4 days, 5 days, 6 days, and/or 7 days after the administration. In some embodiments, the expression of the CPS1 protein is detectable 1 week to 8 weeks after the administration. For example, in some embodiments, CPS1 protein is detectable 1 week, 2 weeks, 3 weeks, and/or 4 weeks after the administration. In some embodiments, the expression of the CPS1 protein is detectable after a month after the administration.

In some embodiments, administering of the composition results in reduced ammonia levels in a subject as compared to baseline levels before treatment. Typically, baseline levels are measured in the subject immediately before treatment. Typically, ammonia levels are measured in a biological sample. Suitable biological samples include, for example, whole blood, plasma, serum, urine or cerebral spinal fluid.

In some embodiments, administering the composition results in reduced ammonia levels in a biological sample (e.g., a serum, plasma, or urine sample) by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% as compared to baseline levels in a subject immediately before treatment.

In some embodiments, administering the composition provided herein results in reduced ammonia levels in plasma or serum as compared to baseline ammonia levels in a subject immediately before treatment. In some embodiments, administering the provided composition results in reduced ammonia levels in plasma or serum as compared to the ammonia levels in subjects who are not treated. In some embodiments, administering the composition results in reduction of ammonia levels to about 3000 μmol/L or less, about 2750 μmol/L or less, about 2500 μmol/L or less, about 2250 μmol/L or less, about 2000 μmol/L or less, about 1750 μmol/L or less, about 1500 μmol/L or less, about 1250 μmol/L or less, about 1000 μmol/L or less, about 750 μmol/L or less, about 500 μmol/L or less, about 250 μmol/L or less, about 100 μmol/L or less or about 50 μmol/L or less in the plasma or serum of the subject. In a particular embodiment, administering the composition results in reduction of ammonia levels to about 50 μmol/L or less in the plasma or serum.

### 3.9 ADAMTS13

In some embodiments, the present invention provides methods and compositions for delivering circRNA encoding ADAMTS13 to a subject for the treatment of thrombotic thrombocytopenic purpura (TTP). A suitable ADAMTS13 circRNA encodes any full length ADAMTS13 protein, or functional fragment or portion thereof, which can be substituted for naturally-occurring ADAMTS13 protein and/or reduce the intensity, severity, and/or frequency of one or more symptoms associated with TTP.

In some embodiments, the RNA sequence of the present invention comprises a circRNA sequence encoding human ADAMTS13 protein.

In some embodiments, the RNA sequence may be an RNA sequence that encodes a homolog or an analog of human ADAMTS13. As used herein, a homolog or an analog of human ADAMTS13 protein may be a modified human ADAMTS13 protein containing one or more amino acid substitutions, deletions, and/or insertions as compared to a wild-type or naturally-occurring human ADAMTS13 protein while retaining substantial ADAMTS13 protein activity.

The ADAMTS13 enzyme cleaves von Willebrand factor, which, in its un-cleaved form, interacts with platelets and causes them to stick together and adhere to the walls of blood vessels, forming clots. Defects in ADAMTS13 are associated with TTP.

In some embodiments, administering the provided composition results in the expression of a ADAMTS13 protein level at or above about 100 ng/mg, about 200 ng/mg, about 300 ng/mg, about 400 ng/mg, about 500 ng/mg, about 600 ng/mg, about 700 ng/mg, about 800 ng/mg, about 900 ng/mg, about 1000 ng/mg, about 1200 ng/mg or about 1400 ng/mg of total protein in the liver.

In some embodiments, the expression of the ADAMTS13 protein is detectable 1 to 96 hours after administration. For example, in some embodiments, expression of ADAMTS13 protein is detectable 1 to 84 hours, 1 to 72 hours, 1 to 60 hours, 1 to 48 hours, 1 to 36 hours, 1 to 24 hours, 1 to 12 hours, 1 to 10 hours, 1 to 8 hours, 1 to 6 hours, 1 to 4 hours, 1 to 2 hours, 2 to 96 hours, 2 to 84 hours, 2 to 72 hours, 2 to 60 hours, 2 to 48 hours, 2 to 36 hours, 2 to 24 hours, 2 to 12 hours, 2 to 10 hours, 2 to 8 hours, 2 to 6 hours, 2 to 4 hours, 4 to 96 hours, 4 to 84 hours, 4 to 72 hours, 4 to 60 hours, 4 to 48 hours, 4 to 36 hours, 4 to 24 hours, 4 to 12 hours, 4 to 10 hours, 4 to 8 hours, 4 to 6 hours, 6 to 96 hours, 6 to 84 hours, 6 to 72 hours, 6 to 60 hours, 6 to 48 hours, 6 to 36 hours, 6 to 24 hours, 6 to 12 hours, 6 to 10 hours, 6 to 8 hours, 8 to 96 hours, 8 to 84 hours, 8 to 72 hours, 8 to 60 hours, 8 to 48 hours, 8 to 36 hours, 8 to 24 hours, 8 to 12 hours, 8 to 10 hours, 10 to 96 hours, 10 to 84 hours, 10 to 72 hours, 10 to 60 hours, 10 to 48 hours, 10 to 36 hours, 10 to 24 hours, 10 to 12 hours, 12 to 96 hours, 12 to 84 hours, 12 to 72 hours, 12 to 60 hours, 12 to 48 hours, 12 to 36 hours, 12 to 24 hours, 24 to 96 hours, 24 to 84 hours, 24 to 72 hours, 24 to 60 hours, 24 to 48 hours, 24 to 36 hours, 36 to 96 hours, 36 to 84 hours, 36 to 72 hours, 36 to 60 hours, 36 to 48 hours, 48 to 96 hours, 48 to 84 hours, 48 to 72 hours, 48 to 60 hours, 48 to 84 hours, 48 to 72 hours, 48 to 60 hours, 60 to 96 hours, 60 to 84 hours, 60 to 72 hours, 72 hours to 96 hours, 72 hours to 84 hours, or 84 hours to 96 hours after administration. For example, in certain embodiments, the expression of the ADAMTS13 protein is detectable 6, 12, 18, 24, 30, 36, 42, 48, 54, 60, 66, and/or 72 hours after the administration. In some embodiments, the expression of the ADAMTS13 protein is detectable 1 day to 7 days after the administration. For example, in some embodiments, ADAMTS13 protein is detectable 1 day, 2 days, 3 days, 4 days, 5 days, 6 days, and/or 7 days after the administration. In some embodiments, the expression of the ADAMTS13 protein is detectable 1 week to 8 weeks after the administration. For example, in some embodiments, ADAMTS13 protein is detectable 1 week, 2 weeks, 3 weeks, and/or 4 weeks after the administration. In some embodiments, the expression of the ADAMTS13 protein is detectable after a month after the administration.

In some embodiments, administering the composition results in reduced von Willebrand factor (vWF) levels in a subject as compared to baseline vWR levels before treatment. Typically, the baseline levels are measured in the subject immediately before treatment. Typically, vWF levels are measured in a biological sample. Suitable biological samples include, for example, whole blood, plasma or serum.

In some embodiments, administering the composition results in reduced vWF levels in a biological sample taken from the subject by at least about 10%, 20%, 30%, 40%, 50%, 60%, 70%, 80%, 90%, or 95% as compared to baseline vWF levels immediately before treatment. In some embodiments, administering the composition results in reduced plasma vWF levels in the subject to less than about 2000 μM, 1500 μM, 1000 μM, 750 μM, 500 μM, 250 μM, 100 μM, 90 μM, 80 μM, 70 μM, 60 μM, 50 μM, 40 μM or 30 μM.

In some embodiments, administering the provided composition results in reduced vWF levels in plasma or serum samples taken from the subject as compared to baseline vWF levels immediately before treatment. In some embodiments, administering the provided composition results in reduced vWF levels in plasma or serum as compared to vWF levels in subjects who are not treated. In some embodiments, administering the composition results in reduction of vWF levels to about 3000 μmol/L or less, about 2750 μmol/L or less, about 2500 μmon or less, about 2250 μmol/L or less, about 2000 μmol/L or less, about 1750 μmol/L or less, about 1500 μmol/L or less, about 1250 μmol/L or less, about 1000 μmol/L or less, about 750 μmol/L or less, about 500 μmol/L or less, about 250 μmol/L or less, about 100 μmol/L or less or about 50 μmol/L or less in the plasma or serum. In a particular embodiment, administering the composition results in reduction of vWF levels to about 50 μmol/L or less in the plasma or serum

### 4. Production of Polynucleotides

The vectors provided herein can be made using standard techniques of molecular biology. For example, the various elements of the vectors provided herein can be obtained using recombinant methods, such as by screening cDNA and genomic libraries from cells, or by deriving the polynucleotides from a vector known to include the same.

The various elements of the vectors provided herein can also be produced synthetically, rather than cloned, based on the known sequences. The complete sequence can be assembled from overlapping oligonucleotides prepared by standard methods and assembled into the complete sequence. See, e.g., Edge, Nature (1981) 292:756; Nambair et al., Science (1984) 223: 1299; and Jay et al., J. Biol. Chem. (1984) 259:631 1.

Thus, particular nucleotide sequences can be obtained from vectors harboring the desired sequences or synthesized completely or in part using various oligonucleotide synthesis techniques known in the art, such as site-directed mutagenesis and polymerase chain reaction (PCR) techniques where appropriate. One method of obtaining nucleotide sequences encoding the desired vector elements is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al., Proc. Natl. Acad. Sci. USA (1991) 88:4084-4088. Additionally, oligonucleotide-directed synthesis (Jones et al., Nature (1986) 54:75-82), oligonucleotide directed mutagenesis of preexisting nucleotide regions (Riechmann et al., Nature (1988) 332:323-327 and Verhoeyen et al., Science (1988) 239: 1534-1536), and enzymatic filling-in of gapped oligonucleotides using T4 DNA polymerase (Queen et al., Proc. Natl. Acad. Sci. USA (1989) 86: 10029-10033) can be used.

The precursor RNA provided herein can be generated by incubating a vector provided herein under conditions permissive of transcription of the precursor RNA encoded by the vector. For example, in some embodiments a precursor RNA is synthesized by incubating a vector provided herein that comprises an RNA polymerase promoter upstream of its 5′ duplex forming region and/or expression sequence with a compatible RNA polymerase enzyme under conditions permissive of in vitro transcription. In some embodiments, the vector is incubated inside of a cell by a bacteriophage RNA polymerase or in the nucleus of a cell by host RNA polymerase II.

In certain embodiments, provided herein is a method of generating precursor RNA by performing in vitro transcription using a vector provided herein as a template (e.g., a vector provided herein with a RNA polymerase promoter positioned upstream of the 5′ homology region).

In certain embodiments, the resulting precursor RNA can be used to generate circular RNA (e.g., a circular RNA polynucleotide provided herein) by incubating it in the presence of magnesium ions and guanosine nucleotide or nucleoside at a temperature at which RNA circularization occurs (e.g., between 20° C. and 60° C.).

Thus, in certain embodiments provided herein is a method of making circular RNA. In certain embodiments, the method comprises synthesizing precursor RNA by transcription (e.g., run-off transcription) using a vector provided herein (e.g., a vector comprising, in the following order, a 5′ homology region, a 3′ group I intron fragment, a first spacer, an Internal Ribosome Entry Site (IRES), an expression sequence, a second spacer, a 5′ group I intron fragment, and a 3′ homology region) as a template, and incubating the resulting precursor RNA in the presence of divalent cations (e.g., magnesium ions) and GTP such that it circularizes to form circular RNA. In some embodiments, the precursor RNA disclosed herein is capable of circularizing in the absence of magnesium ions and GTP and/or without the step of incubation with magnesium ions and GTP. It has been discovered that circular RNA has reduced immunogenicity relative to a corresponding mRNA, at least partially because the mRNA contains an immunogenic 5′ cap. When transcribing a DNA vector from certain promoters (e.g., a T7 promoter) to produce a precursor RNA, it is understood that the 5′ end of the precursor RNA is G. To reduce the immunogenicity of a circular RNA composition that contains a low level of contaminant linear mRNA, an excess of GMP relative to GTP can be provided during transcription such that most transcripts contain a 5′ GMP, which cannot be capped. Therefore, in some embodiments, transcription is carried out in the presence of an excess of GMP. In some embodiments, transcription is carried out where the ratio of GMP concentration to GTP concentration is within the range of about 3:1 to about 15:1, for example, about 3:1 to about 10:1, about 3:1 to about 5:1, about 3:1, about 4:1, or about 5:1.

In some embodiments, a composition comprising circular RNA has been purified. Circular RNA may be purified by any known method commonly used in the art, such as column chromatography, gel filtration chromatography, and size exclusion chromatography. In some embodiments, purification comprises one or more of the following steps: phosphatase treatment, HPLC size exclusion purification, and RNase R digestion. In some embodiments, purification comprises the following steps in order: RNase R digestion, phosphatase treatment, and HPLC size exclusion purification. In some embodiments, purification comprises reverse phase HPLC. In some embodiments, a purified composition contains less double stranded RNA, DNA splints, triphosphorylated RNA, phosphatase proteins, protein ligases, capping enzymes and/or nicked RNA than unpurified RNA. In some embodiments, a purified composition is less immunogenic than an unpurified composition. In some embodiments, immune cells exposed to a purified composition produce less IFN-β1, RIG-I, IL-2, IL-6, IFNγ, and/or TNFα than immune cells exposed to an unpurified composition.

### 5. Ionizable Lipids

In certain embodiments disclosed herein are ionizable lipids that may be used as a component of a transfer vehicle to facilitate or enhance the delivery and release of circular RNA to one or more target cells (e.g., by permeating or fusing with the lipid membranes of such target cells). In certain embodiments, an ionizable lipid comprises one or more cleavable functional groups (e.g., a disulfide) that allow, for example, a hydrophilic functional head-group to dissociate from a lipophilic functional tail-group of the compound (e.g., upon exposure to oxidative, reducing or acidic conditions), thereby facilitating a phase transition in the lipid bilayer of the one or more target cells.

In some embodiments, an ionizable lipid is a lipid as described in international patent application PCT/US2018/058555.

In some of embodiments, a cationic lipid has the following formula:

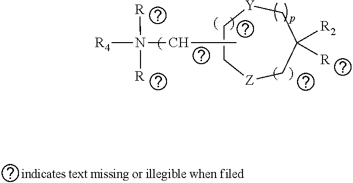

wherein:

R₁ and R₂ are either the same or different and independently optionally substituted C₁₀-C₂₄ alkyl, optionally substituted C₁₀-C₂₄ alkenyl, optionally substituted C₁₀-C₂₄ alkynyl, or optionally substituted C₁₀-C₂₄ acyl;

R₃ and R₄ are either the same or different and independently optionally substituted C₁-C₆ alkyl, optionally substituted C₂-C₆ alkenyl, or optionally substituted C₂-C₆ alkynyl or R₃ and R₄ may join to form an optionally substituted heterocyclic ring of 4 to 6 carbon atoms and 1 or 2 heteroatoms chosen from nitrogen and oxygen;

R₅ is either absent or present and when present is hydrogen or C₁-C₆ alkyl; m, n, and p are either the same or different and independently either 0 or 1 with the proviso that m, n, and p are not simultaneously 0; q is 0, 1, 2, 3, or 4; and

Y and Z are either the same or different and independently O, S, or NH.

In one embodiment, R₁ and R₂ are each linoleyl, and the amino lipid is a dilinoleyl amino lipid.

In one embodiment, the amino lipid is a dilinoleyl amino lipid.

In various other embodiments, a cationic lipid has the following structure:

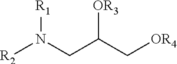

or a pharmaceutically acceptable salt, tautomer, prodrug or stereoisomer thereof, wherein:

R₁ and R₂ are each independently selected from the group consisting of H and C₁-C₃ alkyls; and

R₃ and R₄ are each independently an alkyl group having from about 10 to about 20 carbon atoms, wherein at least one of R₃ and R₄ comprises at least two sites of unsaturation.

In some embodiments, R₃ and R₄ are each independently selected from dodecadienyl, tetradecadienyl, hexadecadienyl, linoleyl, and icosadienyl. In an embodiment, R₃ and R₄ and are both linoleyl. In some embodiments, R₃ and/or R₄ may comprise at least three sites of unsaturation (e.g., R₃ and/or R₄ may be, for example, dodecatrienyl, tetradectrienyl, hexadecatrienyl, linolenyl, and icosatrienyl).

In some embodiments, a cationic lipid has the following structure:

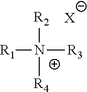

or a pharmaceutically acceptable salt, tautomer, prodrug or stereoisomer thereof, wherein:

R₁ and R₂ are each independently selected from H and C₁-C₃ alkyls;

R₃ and R₄ are each independently an alkyl group having from about 10 to about 20 carbon atoms, wherein at least one of R₃ and R₄ comprises at least two sites of unsaturation.

In one embodiment, R₃ and R₄ are the same, for example, in some embodiments R₃ and R₄ are both linoleyl (C₁₈-alkyl). In another embodiment, R₃ and R₄ are different, for example, in some embodiments, R₃ is tetradectrienyl (C₁₄-alkyl) and R₄ is linoleyl (C₁₈-alkyl). In a preferred embodiment, the cationic lipid(s) of the present invention are symmetrical, i.e., R₃ and R₄ are the same. In another preferred embodiment, both R₃ and R₄ comprise at least two sites of unsaturation. In some embodiments, R₃ and R₄ are each independently selected from dodecadienyl, tetradecadienyl, hexadecadienyl, linoleyl, and icosadienyl. In an embodiment, R₃ and R₄ are both linoleyl. In some embodiments, R₃ and/or R₄ comprise at least three sites of unsaturation and are each independently selected from dodecatrienyl, tetradectrienyl, hexadecatrienyl, linolenyl, and icosatrienyl.

In various embodiments, a cationic lipid has the formula:

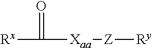

or a pharmaceutically acceptable salt, tautomer, prodrug or stereoisomer thereof, wherein:

Xₐₐ is a D- or L-amino acid residue having the formula —NR^{N}—CR¹R²—C(C═O)—, or a peptide or a peptide of amino acid residues having the formula —{NR^{N}—CR¹R²—C(C═O)}ₙ—, wherein n is an integer from 2 to 20;

R¹ is independently, for each occurrence, a non-hydrogen or a substituted or unsubstituted side chain of an amino acid;

R² and R^{N} are independently, for each occurrence, hydrogen, an organic group consisting of carbon, oxygen, nitrogen, sulfur, and hydrogen atoms, or any combination of the foregoing, and having from 1 to 20 carbon atoms, C₍₁₋₅₎alkyl, cycloalkyl, cycloalkylalkyl, C₍₁₋₅₎alkenyl, C₍₁₋₅₎alkynyl, C₍₁₋₅₎alkanoyl, C₍₁₋₅₎alkanoyloxy, C₍₁₋₅₎alkoxy, C₍₁₋₅₎alkoxy-C₍₁₋₅₎alkyl, C₍₁₋₅₎alkoxy-C₍₁₋₅₎alkoxy, C₍₁₋₅₎alkyl-amino-C₍₁₋₅₎alkyl-, C₍₁₋₅₎dialkyl-amino-C₍₁₋₅₎alkyl-, nitro-C₍₁₋₅₎alkyl, cyano-C₍₁₋₅₎alkyl, aryl-C₍₁₋₅₎alkyl, 4-biphenyl-C₍₁₋₅₎alkyl, carboxyl, or hydroxyl;

Z is —NH—, O, S, CH₂S—, —CH₂S(O)—, or an organic linker consisting of 1-40 atoms selected from hydrogen, carbon, oxygen, nitrogen, and sulfur atoms (preferably, Z is —NH— or —O—);

R^{x} and R^{y} are, independently, (i) a lipophilic tail derived from a lipid (which can be naturally occurring or synthetic), e.g., a phospholipid, a glycolipid, a triacylglycerol, a glycerophospholipid, a sphingolipid, a ceramide, a sphingomyelin, a cerebroside, or a ganglioside, wherein the tail optionally includes a steroid; (ii) an amino acid terminal group selected from hydrogen, hydroxyl, amino, and an organic protecting group; or (iii) a substituted or unsubstituted C₍₃₋₂₂₎alkyl, C₍₆₋₁₂₎cycloalkyl, C₍₆₋₁₂₎cycloalkyl-C₍₃₋₂₂₎alkyl, C₍₁₋₂₂₎alkenyl, C₍₃₋₂₂₎alkynyl, C₍₃₋₂₂₎alkoxy, or C₍₆₋₁₂₎-alkoxy C₍₃₋₂₂₎alkyl;

In some embodiments, one of R^{x} and R^{y} is a lipophilic tail as defined above and the other is an amino acid terminal group. In some embodiments, both R^{x} and R^{y} are lipophilic tails.

In some embodiments, at least one of R^{x} and R^{y} is interrupted by one or more biodegradable groups (e.g., —OC(O)—, —C(O)O—, —SC(O)—, —C(O)S—, —OC(S)—, —C(S)O—, —S—S—, —C(O)(NR⁵)—, —N(R⁵)C(O)—, —C(S)(NR⁵)—, —N(R⁵)C(O)—, —N(R⁵)C(O)N(R⁵)—, —OC(O)O—, —OSi(R⁵)₂O—, —C(O)(CR³R⁴)C(O)O—, —OC(O)(CR³R⁴)C(O)—, or

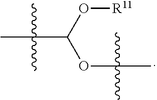

In some embodiments, R¹ is a C₂-C₈alkyl or alkenyl.

In some embodiments, each occurrence of R⁵ is, independently, H or alkyl.

In some embodiments, each occurrence of R³ and R⁴ are, independently H, halogen, OH, alkyl, alkoxy, —NH₂, alkylamino, or dialkylamino; or R₃ and R₄, together with the carbon atom to which they are directly attached, form a cycloalkyl group. In some particular embodiments, each occurrence of R³ and R⁴ are, independently H or C₁-C₄alkyl.

In some embodiments, R^{x} and R^{y} each, independently, have one or more carbon-carbon double bonds.

In some embodiments, the cationic lipid is one of the following:

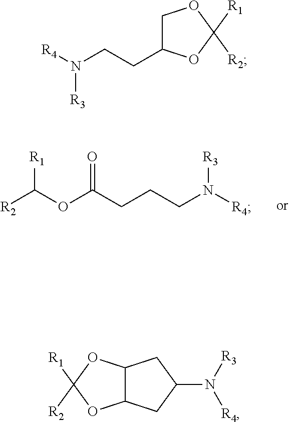

or a pharmaceutically acceptable salt, tautomer, prodrug or stereoisomer thereof, wherein:

R₁ and R₂ are each independently alkyl, alkenyl, or alkynyl, each of which can optionally substituted;

R₃ and R₄ are each independently a C₁-C₆ alkyl, or R₃ and R₄ are taken together to form an optionally substituted heterocyclic ring.

A representative useful dilinoleyl amino lipid has the formula:

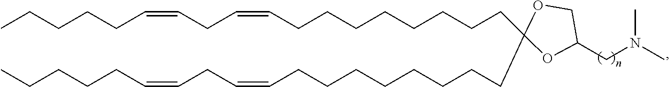

wherein n is 0, 1, 2, 3, or 4.

In one embodiment, a cationic lipid is DLin-K-DMA. In one embodiment, a cationic lipid is DLin-KC2-DMA (DLin-K-DMA above, wherein n is 2).

In one embodiment, a cationic lipid has the following structure:

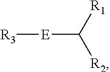

or a pharmaceutically acceptable salt, tautomer, prodrug or stereoisomer thereof, wherein:

R₁ and R₂ are each independently for each occurrence optionally substituted C₁₀-C₃₀ alkyl, optionally substituted C₁₀-C₃₀ alkenyl, optionally substituted C₁₀-C₃₀ alkynyl or optionally substituted C₁₀-C₃₀ acyl;

R₃ is H, optionally substituted C₂-C₁₀ alkyl, optionally substituted C₂-C₁₀ alkenyl, optionally substituted C₂-C₁₀ alkylyl, alkylhetrocycle, alkylphosphate, alkylphosphorothioate, alkylphosphorodithioate, alkylphosphonate, alkylamine, hydroxyalkyl, ω-aminoalkyl, ω-(substituted)aminoalkyl, ω-phosphoalkyl, ω-thiophosphoalkyl, optionally substituted polyethylene glycol (PEG, mw 100-40K), optionally substituted mPEG (mw 120-40K), heteroaryl, or heterocycle, or a linker ligand, for example, in some embodiments, R₃ is (CH₃)₂N(CH₂)ₙ—, wherein n is 1, 2, 3 or 4;

E is O, S, N(Q), C(O), OC(O), C(O)O, N(Q)C(O), C(O)N(Q), (Q)N(CO)O, O(CO)N(Q), S(O), NS(O)₂N(Q), S(O)₂, N(Q)S(O)₂, SS, O═N, aryl, heteroaryl, cyclic or heterocycle, for example —C(O)O, wherein — is a point of connection to R₃; and

Q is H, alkyl, ω-aminoalkyl, ω-(substituted)aminoalkyl, ω-phosphoalkyl or ω-thiophosphoalkyl.

In one specific embodiment, the cationic lipid of Embodiments 1, 2, 3, 4 or 5 has the following structure:

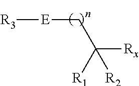

or a pharmaceutically acceptable salt, tautomer, prodrug or stereoisomer thereof, wherein:

E is O, S, N(Q), C(O), N(Q)C(O), C(O)N(Q), (Q)N(CO)O, O(CO)N(Q), S(O), NS(O)₂N(Q), S(O)₂, N(Q)S(O)₂, SS, O═N, aryl, heteroaryl, cyclic or heterocycle;

Q is H, alkyl, ω-aminoalkyl, ω-(substituted)aminoalkyl, ω-phosphoalkyl or ω-thiophosphoalkyl;

R₁ and R₂ and Rₓ are each independently for each occurrence H, optionally substituted C₁-C₁₀ alkyl, optionally substituted C₁₀-C₃₀ alkyl, optionally substituted C₁₀-C₃₀ alkenyl, optionally substituted C₁₀-C₃₀alkynyl, optionally substituted C₁₀-C₃₀ acyl, or linker-ligand, provided that at least one of R₁, R₂ and Rₓ is not

R₃ is optionally substituted C₁-C₁₀ alkyl, optionally substituted C₂-C₁₀ alkenyl, optionally substituted C₂-C₁₀ alkynyl, alkylhetrocycle, alkylphosphate, alkylphosphorothioate, alkylphosphorodithioate, alkylphosphonate, alkylamine, hydroxyalkyl, ω-aminoalkyl, ω-(substituted)aminoalkyl, ω-phosphoalkyl, ω-thiophosphoalkyl, optionally substituted polyethylene glycol (PEG, mw 100-40K), optionally substituted mPEG (mw 120-40K), heteroaryl, or heterocycle, or linker-ligand; and

n is 0, 1, 2, or 3,

In one embodiment, the cationic lipid of Embodiments 1, 2, 3, 4 or 5 has the structure of Formula I:

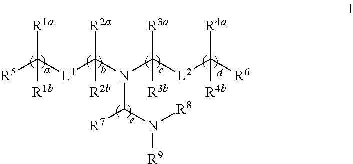

or a pharmaceutically acceptable salt, tautomer, prodrug or stereoisomer thereof, wherein:

one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or

—NR^{a}C(═O)O— or a direct bond;

R^{a} is H or C₁-C₁₂ alkyl;

R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;

R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;

R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;

R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;

R⁵ and R⁶ are each independently methyl or cycloalkyl;

R⁷ is, at each occurrence, independently H or C₁-C₁₂ alkyl;

R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;

a and d are each independently an integer from 0 to 24;

b and c are each independently an integer from 1 to 24;

e is 1 or 2; and

x is 0, 1 or 2.

In some embodiments of Formula I, L¹ and L² are independently —O(C═O)— or —(C═O)O—.

In certain embodiments of Formula I, at least one of R₁ₐ, R^{2a}, R^{3a} or R^{4a} is C₁-C₁₂ alkyl, or at least one of L¹ or L² is —O(C═O)— or —(C═O)O—. In other embodiments, R^{1a} and R^{1b} are not isopropyl when a is 6 or n-butyl when a is 8.

In still further embodiments of Formula I, at least one of R^{1a}, R^{2a}, R^{3a} or R^{4a} is C₁-C₁₂ alkyl, or at least one of L¹ or L² is —O(C═O)— or —(C═O)O—; and

R^{1a} and R^{1b} are not isopropyl when a is 6 or n-butyl when a is 8.

In other embodiments of Formula I, R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;

In certain embodiments of Formula I, any one of L¹ or L² may be —O(C═O)— or a carbon-carbon double bond. L¹ and L² may each be —O(C═O)— or may each be a carbon-carbon double bond.

In some embodiments of Formula I, one of L¹ or L² is —O(C═O)—. In other embodiments, both L¹ and L² are —O(C═O)—.

In some embodiments of Formula I, one of L¹ or L² is —(C═O)O—. In other embodiments, both L¹ and L² are —(C═O)O—.

In some other embodiments of Formula I, one of L¹ or L² is a carbon-carbon double bond. In other embodiments, both L¹ and L² are a carbon-carbon double bond.

In still other embodiments of Formula I, one of L¹ or L² is —O(C═O)— and the other of L¹ or L² is —(C═O)O—. In more embodiments, one of L¹ or L² is —O(C═O)— and the other of L¹ or L² is a carbon-carbon double bond. In yet more embodiments, one of L¹ or L² is —(C═O)O— and the other of L¹ or L² is a carbon-carbon double bond.

It is understood that “carbon-carbon” double bond, as used throughout the specification, refers to one of the following structures:

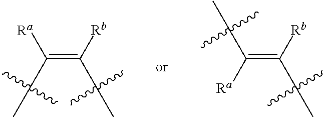

wherein R^{a} and R^{b} are, at each occurrence, independently H or a substituent. For example, in some embodiments R^{a} and R^{b} are, at each occurrence, independently H, C₁-C₁₂ alkyl or cycloalkyl, for example H or C₁-C₁₂ alkyl.

In other embodiments, the lipid compounds of Formula I have the following Formula (Ia):

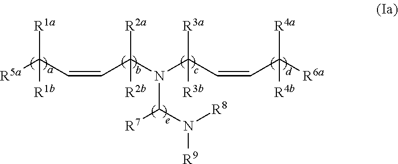

In other embodiments, the lipid compounds of Formula I have the following Formula (Ib):

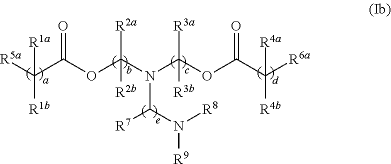

In yet other embodiments, the lipid compounds of Formula I have the following Formula (Ic):

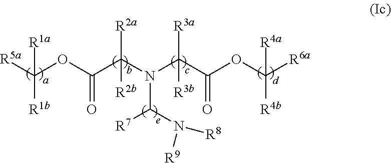

In certain embodiments of the lipid compound of Formula I, a, b, c and d are each independently an integer from 2 to 12 or an integer from 4 to 12. In other embodiments, a, b, c and d are each independently an integer from 8 to 12 or 5 to 9. In some certain embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some other embodiments of Formula I, b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some more embodiments of Formula I, c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some certain other embodiments of Formula I, d is 0. In some embodiments, d is 1. In other embodiments, d is 2. In more embodiments, d is 3. In yet other embodiments, d is 4. In some embodiments, d is 5. In other embodiments, d is 6. In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some other various embodiments of Formula I, a and d are the same. In some other embodiments, b and c are the same. In some other specific embodiments, a and d are the same and b and c are the same.

The sum of a and b and the sum of c and d in Formula I are factors which may be varied to obtain a lipid of formula I having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a. b, c and d are selected such the sum of a and b and the sum of c and d is 12 or greater.

In some embodiments of Formula I, e is 1. In other embodiments, e is 2.

The substituents at R^{1a}, R^{2a}, R^{3a} and R^{4a} of Formula I are not particularly limited. In certain embodiments R^{1a}, R^{2a}, R^{3a} and R^{4a} are H at each occurrence. In certain other embodiments at least one of R^{1a}, R^{2a}, R^{3a} and R^{4a} is C₁-C₁₂ alkyl. In certain other embodiments at least one of R^{1a}, R^{2a}, R^{3a} and R^{4a} is C₁-C₈ alkyl. In certain other embodiments at least one of R^{1a}, R^{2a}, R^{3a} and R^{4a} is C₁-C₆ alkyl. In some of the foregoing embodiments, the C₁-C₈ alkyl is methyl, ethyl, n-propyl, iso-propyl, n-butyl, iso-butyl, tert-butyl, n-hexyl or n-octyl.

In certain embodiments of Formula I, R^{1a}, R^{1b}, R^{4a} and R^{4b} are C₁-C₁₂ alkyl at each occurrence.

In further embodiments of Formula I, at least one of R^{1b}, R^{2b}, R^{3b} and R^{4b} is H or R^{1b}, R^{2b}, R^{3b} and R^{4b} are H at each occurrence.

In certain embodiments of Formula I, R^{1b} together with the carbon atom to which it is bound is taken together with an adjacent R^{1b} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R⁵ and R⁶ of Formula I are not particularly limited in the foregoing embodiments. In certain embodiments one or both of R⁵ or R⁶ is methyl. In certain other embodiments one or both of R⁵ or R⁶ is cycloalkyl for example cyclohexyl. In these embodiments the cycloalkyl may be substituted or not substituted. In certain other embodiments the cycloalkyl is substituted with C₁-C₁₂ alkyl, for example tert-butyl.

The substituents at R′ are not particularly limited in the foregoing embodiments of Formula I. In certain embodiments at least one R⁷ is H. In some other embodiments, R⁷ is H at each occurrence. In certain other embodiments R⁷ is C₁-C₁₂ alkyl.

In certain other of the foregoing embodiments of Formula I, one of R⁸ or R⁹ is methyl. In other embodiments, both R⁸ and R⁹ are methyl.

In some different embodiments of Formula I, R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring.

In some embodiments of Embodiment 3, the first and second cationic lipids are each, independently selected from a lipid of Formula I.

In various different embodiments, the lipid of Formula I has one of the structures set forth in Table 1 below.

TABLE 1
Representative Lipids of Formula I

| No. | Structure | pKa |
|---|---|---|
| I-1 | 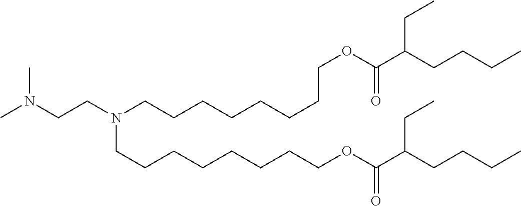 [SMILES: CCCCC(CC)C(=O)OCCCCCCCCN(CCCCCCCCOC(=O)C(CC)CCCC)CCN(C)C] | — |
| I-2 | 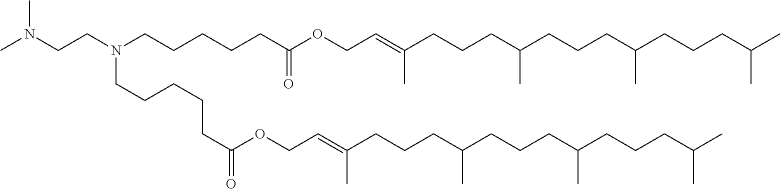 [SMILES: C/C(=C\COC(=O)CCCCCN(CCCCCC(=O)OC/C=C(\C)CCCC(C)CCCC(C)CCCC(C)C)CCN(C)C)CCCC(C)CCCC(C)CCCC(C)C] | 5.64 |
| I-3 | 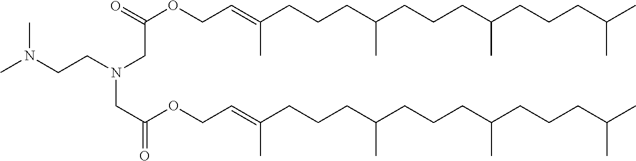 [SMILES: C/C(=C\COC(=O)CN(CCN(C)C)CC(=O)OC/C=C(\C)CCCC(C)CCCC(C)CCCC(C)C)CCCC(C)CCCC(C)CCCC(C)C] | 7.15 |
| I-4 | 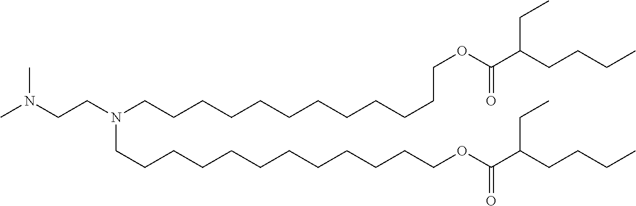 [SMILES: CCCCC(CC)C(=O)OCCCCCCCCCCCCN(CCCCCCCCCCCCOC(=O)C(CC)CCCC)CCN(C)C] | 6.43 |
| I-5 | 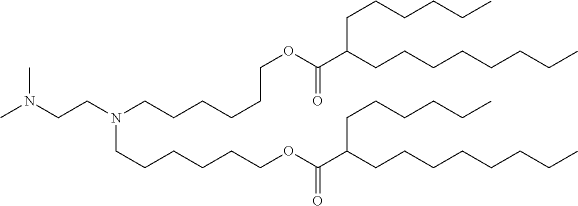 [SMILES: CCCCCCCCC(CCCCCC)C(=O)OCCCCCCN(CCCCCCOC(=O)C(CCCCCC)CCCCCCCC)CCN(C)C] | 6.28 |
| I-6 | 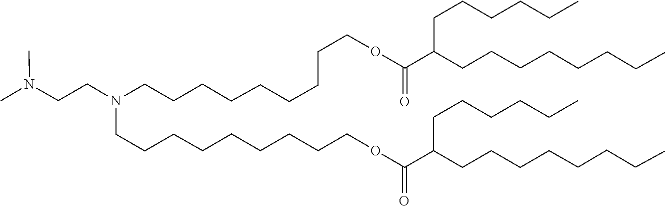 [SMILES: CCCCCCCCC(CCCCCC)C(=O)OCCCCCCCCCN(CCCCCCCCCOC(=O)C(CCCCCC)CCCCCCCC)CCN(C)C] | 6.12 |
| I-7 | 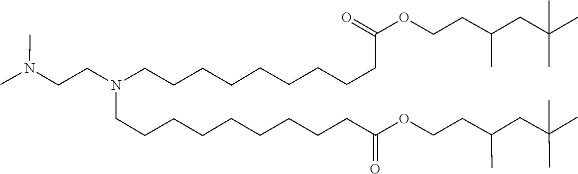 [SMILES: CC(CCOC(=O)CCCCCCCCCN(CCCCCCCCCC(=O)OCCC(C)CC(C)(C)C)CCN(C)C)CC(C)(C)C] | — |
| I-8 | 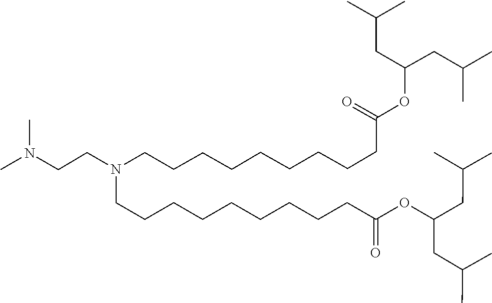 [SMILES: CC(C)CC(CC(C)C)OC(=O)CCCCCCCCCN(CCCCCCCCCC(=O)OC(CC(C)C)CC(C)C)CCN(C)C] | — |
| I-9 | 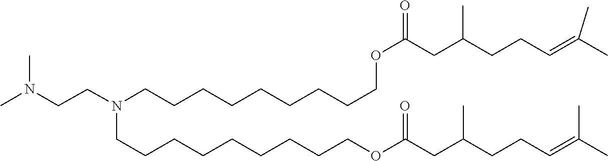 [SMILES: CC(C)=CCCC(C)CC(=O)OCCCCCCCCCN(CCCCCCCCCOC(=O)CC(C)CCC=C(C)C)CCN(C)C] | — |
| I-10 | 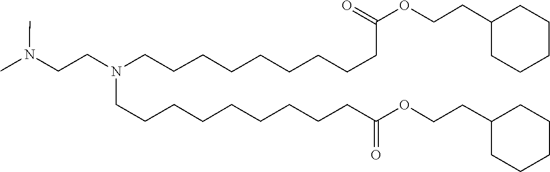 [SMILES: CN(C)CCN(CCCCCCCCCC(=O)OCCC1CCCCC1)CCCCCCCCCC(=O)OCCC1CCCCC1] | — |
| I-11 | 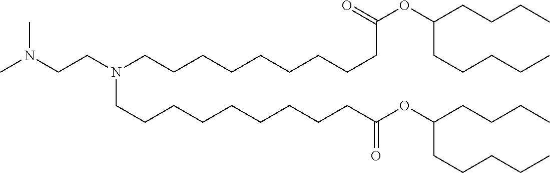 [SMILES: CCCCCC(CCCC)OC(=O)CCCCCCCCCN(CCCCCCCCCC(=O)OC(CCCC)CCCCC)CCN(C)C] | 6.36 |
| I-12 | 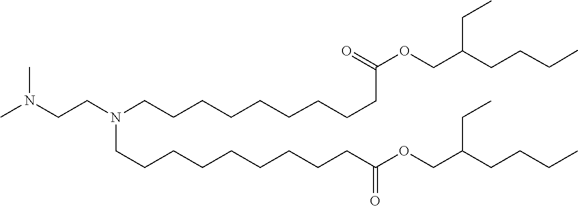 [SMILES: CCCCC(CC)COC(=O)CCCCCCCCCN(CCCCCCCCCC(=O)OCC(CC)CCCC)CCN(C)C] | — |
| I-13 | 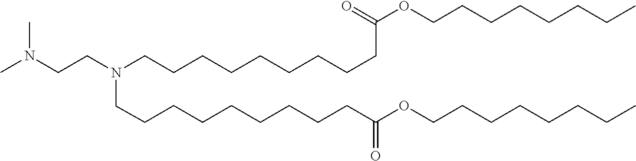 [SMILES: CCCCCCCCOC(=O)CCCCCCCCCN(CCCCCCCCCC(=O)OCCCCCCCC)CCN(C)C] | 6.51 |
| I-14 | 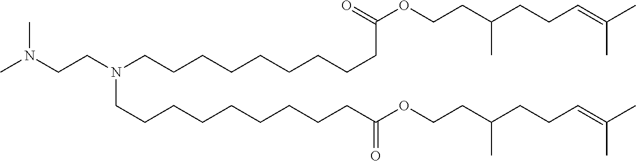 [SMILES: CC(C)=CCCC(C)CCOC(=O)CCCCCCCCCN(CCCCCCCCCC(=O)OCCC(C)CCC=C(C)C)CCN(C)C] | — |
| I-15 | 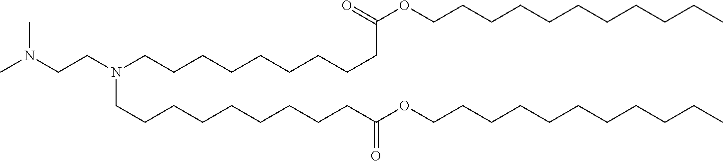 [SMILES: CCCCCCCCCCCOC(=O)CCCCCCCCCN(CCCCCCCCCC(=O)OCCCCCCCCCCC)CCN(C)C] | 6.30 |
| I-16 | 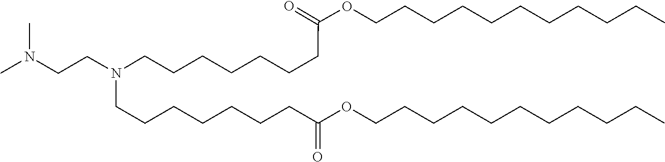 [SMILES: CCCCCCCCCCCOC(=O)CCCCCCCN(CCCCCCCC(=O)OCCCCCCCCCCC)CCN(C)C] | 6.63 |
| I-17 | 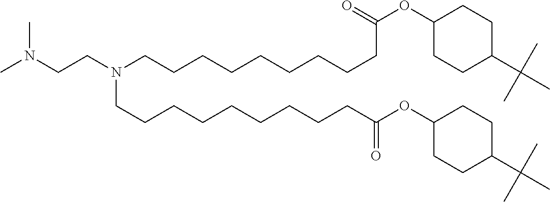 [SMILES: CN(C)CCN(CCCCCCCCCC(=O)OC1CCC(C(C)(C)C)CC1)CCCCCCCCCC(=O)OC1CCC(C(C)(C)C)CC1] | — |
| I-18 | 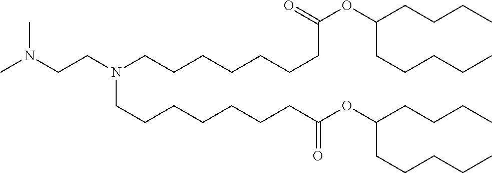 [SMILES: CCCCCC(CCCC)OC(=O)CCCCCCCN(CCCCCCCC(=O)OC(CCCC)CCCCC)CCN(C)C] | — |
| I-19 | 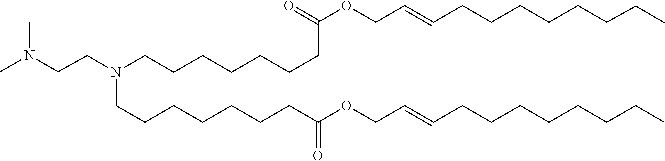 [SMILES: CCCCCCCC/C=C/COC(=O)CCCCCCCN(CCCCCCCC(=O)OC/C=C/CCCCCCCC)CCN(C)C] | 6.72 |
| I-20 | 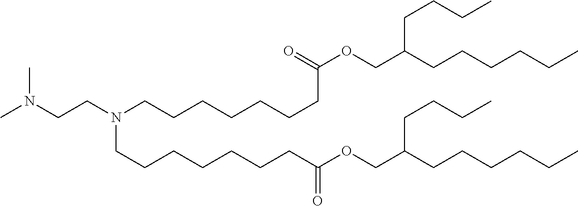 [SMILES: CCCCCCC(CCCC)COC(=O)CCCCCCCN(CCCCCCCC(=O)OCC(CCCC)CCCCCC)CCN(C)C] | 6.44 |
| I-21 | 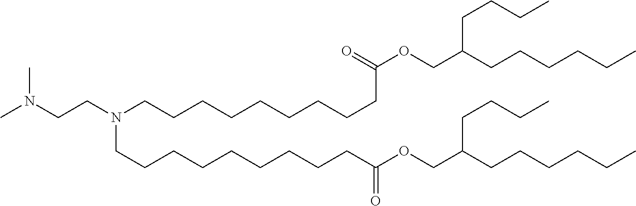 [SMILES: CCCCCCC(CCCC)COC(=O)CCCCCCCCCN(CCCCCCCCCC(=O)OCC(CCCC)CCCCCC)CCN(C)C] | 6.28 |
| I-22 | 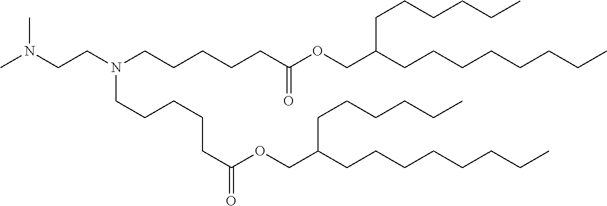 [SMILES: CCCCCCCCC(CCCCCC)COC(=O)CCCCCN(CCCCCC(=O)OCC(CCCCCC)CCCCCCCC)CCN(C)C] | 6.53 |
| I-23 | 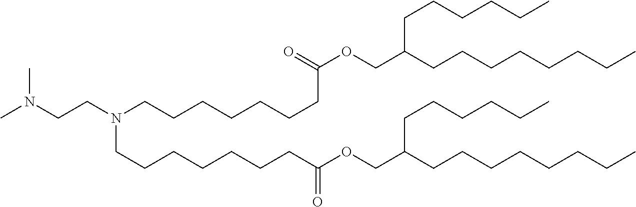 [SMILES: CCCCCCCCC(CCCCCC)COC(=O)CCCCCCCN(CCCCCCCC(=O)OCC(CCCCCC)CCCCCCCC)CCN(C)C] | 6.24 |
| I-24 | 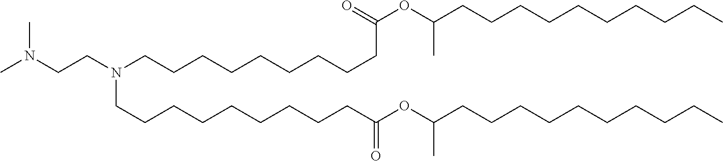 [SMILES: CCCCCCCCCCC(C)OC(=O)CCCCCCCCCN(CCCCCCCCCC(=O)OC(C)CCCCCCCCCC)CCN(C)C] | 6.28 |
| I-25 | 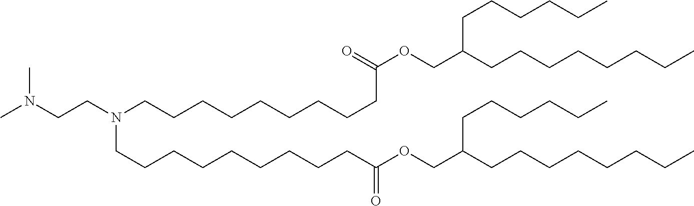 [SMILES: CCCCCCCCC(CCCCCC)COC(=O)CCCCCCCCCN(CCCCCCCCCC(=O)OCC(CCCCCC)CCCCCCCC)CCN(C)C] | 6.20 |
| I-33 | 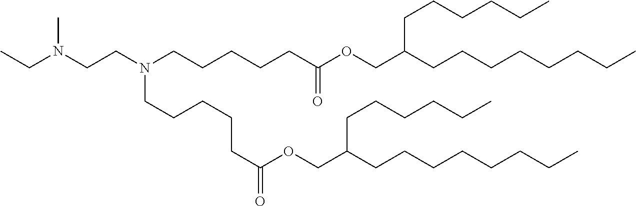 [SMILES: CCCCCCCCC(CCCCCC)COC(=O)CCCCCN(CCCCCC(=O)OCC(CCCCCC)CCCCCCCC)CCN(C)CC] | 6.27 |
| I-34 | 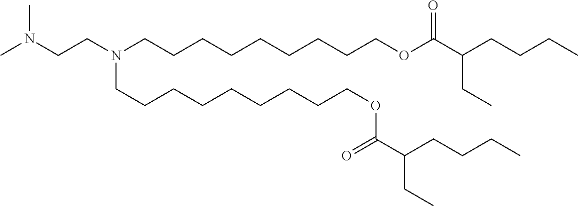 [SMILES: CCCCC(CC)C(=O)OCCCCCCCCCN(CCCCCCCCCOC(=O)C(CC)CCCC)CCN(C)C] | — |
| I-35 | 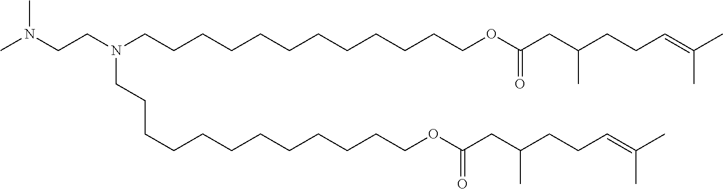 [SMILES: CC(C)=CCCC(C)CC(=O)OCCCCCCCCCCCCN(CCCCCCCCCCCCOC(=O)CC(C)CCC=C(C)C)CCN(C)C] | 6.21 |
| I-36 | 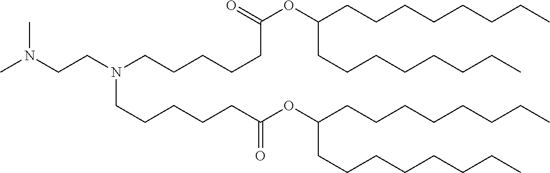 [SMILES: CCCCCCCCC(CCCCCCCC)OC(=O)CCCCCN(CCCCCC(=O)OC(CCCCCCCC)CCCCCCCC)CCN(C)C] | — |
| I-37 | 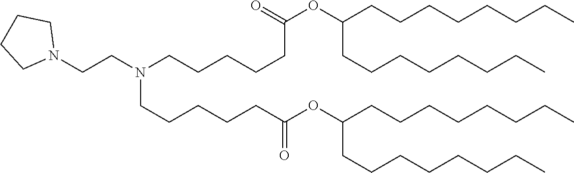 [SMILES: CCCCCCCCC(CCCCCCCC)OC(=O)CCCCCN(CCCCCC(=O)OC(CCCCCCCC)CCCCCCCC)CCN1CCCC1] | — |
| I-38 | 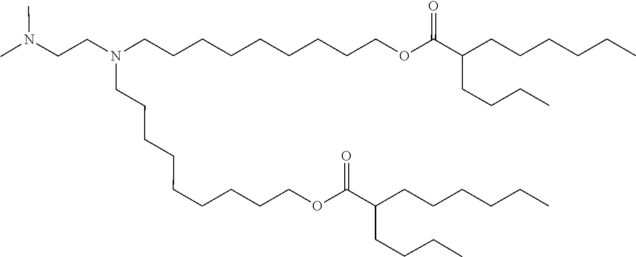 [SMILES: CCCCCCC(CCCC)C(=O)OCCCCCCCCCN(CCCCCCCCCOC(=O)C(CCCC)CCCCCC)CCN(C)C] | 6.24 |
| I-39 | 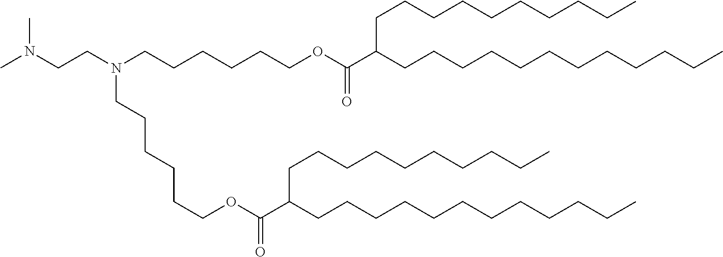 [SMILES: CCCCCCCCCCCCC(CCCCCCCCCC)C(=O)OCCCCCCN(CCCCCCOC(=O)C(CCCCCCCCCC)CCCCCCCCCCCC)CCN(C)C] | 5.82 |
| I-40 | 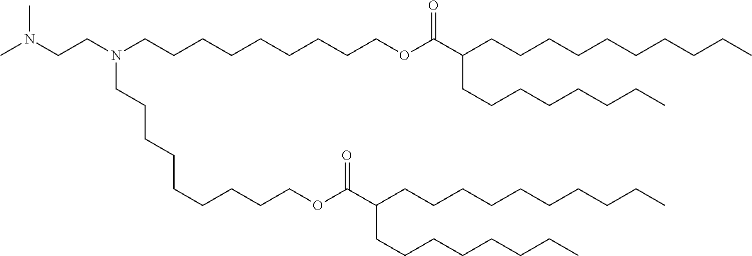 [SMILES: CCCCCCCCCCC(CCCCCCCC)C(=O)OCCCCCCCCCN(CCCCCCCCCOC(=O)C(CCCCCCCC)CCCCCCCCCC)CCN(C)C] | 6.38 |
| I-41 | 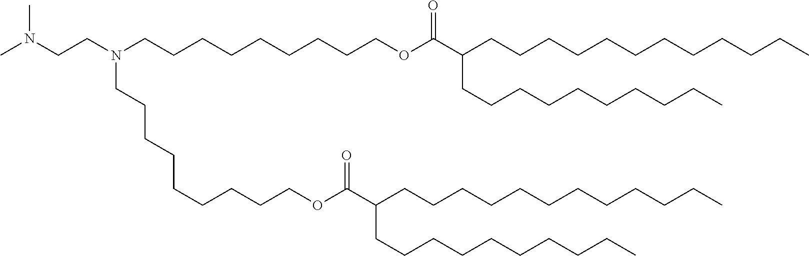 [SMILES: CCCCCCCCCCCCC(CCCCCCCCCC)C(=O)OCCCCCCCCCN(CCCCCCCCCOC(=O)C(CCCCCCCCCC)CCCCCCCCCCCC)CCN(C)C] | 5.91 |

In some embodiments, the cationic lipid of Embodiments 1, 2, 3, 4 or 5 has a structure of Formula II:

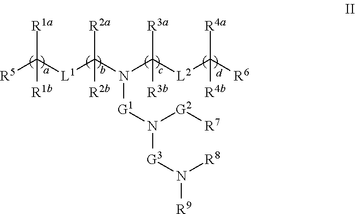

or a pharmaceutically acceptable salt, tautomer, prodrug or stereoisomer thereof, wherein:

one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or

—NR^{a}C(═O)O— or a direct bond;

G¹ is C₁-C₂ alkylene, —(C═O)—, —O(C═O)—, —SC(═O)—, —NR^{a}C(═O)— or a direct bond;

G² is —C(═O)—, —(C═O)O—, —C(═O)S—, —C(═O)NR^{a}— or a direct bond;

G³ is C₁-C₆ alkylene;

R^{a} is H or C₁-C₁₂ alkyl;

R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;

R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;

R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;

R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;

R⁵ and R⁶ are each independently H or methyl;

R⁷ is C₄-C₂₀ alkyl;

R⁸ and R⁹ are each independently C₁-C₁₂ alkyl; or R⁸ and R⁹, together with the nitrogen atom to which they are attached, form a 5, 6 or 7-membered heterocyclic ring;

a, b, c and d are each independently an integer from 1 to 24; and

x is 0, 1 or 2.

In some embodiments of Formula (II), L¹ and L² are each independently —O(C═O)—, —(C═O)O— or a direct bond. In other embodiments, G¹ and G² are each independently —(C═O)— or a direct bond. In some different embodiments, L¹ and L² are each independently —O(C═O)—, —(C═O)O— or a direct bond; and G¹ and G² are each independently —(C═O)— or a direct bond.

In some different embodiments of Formula (II), L¹ and L² are each independently —C(═O)—, —O—, —S(O)ₓ, —S—S—, —C(═O)S—, —SC(═O)—, —NR^{a}—, —NR^{a}C(═O)—, —C(═O)NR^{a}—, —NR^{a}C(═O)NR^{a}, —OC(═O)NR^{a}—, —NR^{a}C(═O)O—, —NR^{a}S(O)ₓNR^{a}—, —NR^{a}S(O)ₓ— or —S(O)ₓNR^{a}—.

In other of the foregoing embodiments of Formula (II), the lipid compound has one of the following Formulae (IIA) or (IIB):

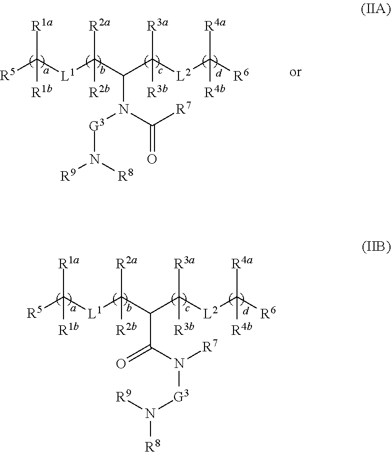

In some embodiments of Formula (II), the lipid compound has Formula (IIA). In other embodiments, the lipid compound has Formula (IIB).

In any of the foregoing embodiments of Formula (II), one of L¹ or L² is —O(C═O)—. For example, in some embodiments each of L¹ and L² are —O(C═O)—.

In some different embodiments of Formula (II), one of L¹ or L² is —(C═O)O—. For example, in some embodiments each of L¹ and L² is —(C═O)O—.

In different embodiments of Formula (II), one of L¹ or L² is a direct bond. As used herein, a “direct bond” means the group (e.g., L¹ or L²) is absent. For example, in some embodiments each of L¹ and L² is a direct bond.

In other different embodiments of Formula (II), for at least one occurrence of R^{1a} and R^{1b}, R^{1a} is H or C₁-C₁₂ alkyl, and R^{1b} together with the carbon atom to which it is bound is taken together with an adjacent R^{1b} and the carbon atom to which it is bound to form a carbon-carbon double bond.

In still other different embodiments of Formula (II), for at least one occurrence of R^{4a} and R^{4b}, R^{4a} is H or C₁-C₁₂ alkyl, and R^{4b} together with the carbon atom to which it is bound is taken together with an adjacent R^{4b} and the carbon atom to which it is bound to form a carbon-carbon double bond.

In more embodiments of Formula (II), for at least one occurrence of R^{2a} and R^{2b}, R^{2a} is H or C₁-C₁₂ alkyl, and R^{2b} together with the carbon atom to which it is bound is taken together with an adjacent R_{2b} and the carbon atom to which it is bound to form a carbon-carbon double bond.

In other different embodiments of Formula (II), for at least one occurrence of R^{3a} and R^{3b}, R^{3a} is H or C₁-C₁₂ alkyl, and R^{3b} together with the carbon atom to which it is bound is taken together with an adjacent R^{3b} and the carbon atom to which it is bound to form a carbon-carbon double bond.

In various other embodiments of Formula (II), the lipid compound has one of the following Formulae (IIC) or (IID):

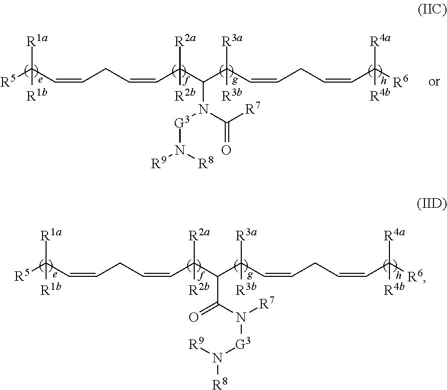

wherein e, f, g and h are each independently an integer from 1 to 12.

In some embodiments of Formula (II), the lipid compound has Formula (IIC). In other embodiments, the lipid compound has Formula (IID).

In various embodiments of Formulae (IIC) or (IID), e, f, g and h are each independently an integer from 4 to 10.

In certain embodiments of Formula (II), a, b, c and d are each independently an integer from 2 to 12 or an integer from 4 to 12. In other embodiments, a, b, c and d are each independently an integer from 8 to 12 or 5 to 9. In some certain embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some embodiments of Formula (II), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some embodiments of Formula (II), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some certain embodiments of Formula (II), d is 0. In some embodiments, d is 1. In other embodiments, d is 2. In more embodiments, d is 3. In yet other embodiments, d is 4. In some embodiments, d is 5. In other embodiments, d is 6. In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some embodiments of Formula (II), e is 1. In other embodiments, e is 2. In more embodiments, e is 3. In yet other embodiments, e is 4. In some embodiments, e is 5. In other embodiments, e is 6. In more embodiments, e is 7. In yet other embodiments, e is 8. In some embodiments, e is 9. In other embodiments, e is 10. In more embodiments, e is 11. In yet other embodiments, e is 12.

In some embodiments of Formula (II), f is 1. In other embodiments, f is 2. In more embodiments, f is 3. In yet other embodiments, f is 4. In some embodiments, f is 5. In other embodiments, f is 6. In more embodiments, f is 7. In yet other embodiments, f is 8. In some embodiments, f is 9. In other embodiments, f is 10. In more embodiments, f is 11. In yet other embodiments, f is 12.

In some embodiments of Formula (II), g is 1. In other embodiments, g is 2. In more embodiments, g is 3. In yet other embodiments, g is 4. In some embodiments, g is 5. In other embodiments, g is 6. In more embodiments, g is 7. In yet other embodiments, g is 8. In some embodiments, g is 9. In other embodiments, g is 10. In more embodiments, g is 11. In yet other embodiments, g is 12.

In some embodiments of Formula (II), h is 1. In other embodiments, e is 2. In more embodiments, h is 3. In yet other embodiments, h is 4. In some embodiments, e is 5. In other embodiments, h is 6. In more embodiments, h is 7. In yet other embodiments, h is 8. In some embodiments, h is 9. In other embodiments, h is 10. In more embodiments, his 11. In yet other embodiments, his 12.

In some other various embodiments of Formula (II), a and d are the same. In some other embodiments, b and c are the same. In some other specific embodiments and a and d are the same and b and c are the same.

The sum of a and b and the sum of c and d of Formula (II) are factors which may be varied to obtain a lipid having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a. b, c and d are selected such that the sum of a and b and the sum of c and d is 12 or greater.

The substituents at R^{1a}, R^{2a}, R^{3a} and R^{4a} of Formula (II) are not particularly limited. In some embodiments, at least one of R^{1a}, R^{2a}, R^{3a} and R^{4a} is H. In certain embodiments R^{1a}, R^{2a}, R^{3a} and R^{4a} are H at each occurrence. In certain other embodiments at least one of R^{1a}, R^{2a}, R^{3a} and R^{4a} is C₁-C₁₂ alkyl. In certain other embodiments at least one of R^{1a}, R^{2a}, R^{3a} and R^{4a} is C₁-C₈ alkyl. In certain other embodiments at least one of R^{1a}, R^{2a}, R^{3a} and R^{4a} is C₁-C₆ alkyl. In some of the foregoing embodiments, the C₁-C₈ alkyl is methyl, ethyl, n-propyl, iso-propyl, n-butyl, iso-butyl, tert-butyl, n-hexyl or n-octyl.

In certain embodiments of Formula (II), R^{1a}, R^{1b}, R^{4a} and R^{4b} are C₁-C₁₂ alkyl at each occurrence.

In further embodiments of Formula (II), at least one of R^{1b}, R^{2b}, R^{3b} and R^{4b} is H or R^{1b}, R^{2b}, R^{3b} and R^{4b} are H at each occurrence.

In certain embodiments of Formula (II), R_{1b} together with the carbon atom to which it is bound is taken together with an adjacent R^{1b} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R⁵ and R⁶ of Formula (II) are not particularly limited in the foregoing embodiments. In certain embodiments one of R⁵ or R⁶ is methyl. In other embodiments each of R⁵ or R⁶ is methyl.

The substituents at R⁷ of Formula (II) are not particularly limited in the foregoing embodiments. In certain embodiments R⁷ is C₆-C₁₆ alkyl. In some other embodiments, R⁷ is C₆-C₉ alkyl. In some of these embodiments, R⁷ is substituted with —(C═O)OR^{b}, —O(C═O)R^{b}, —C(═O)R^{b}, —OR^{b}, —S(O)ₓR^{b}, —S—SR^{b}, —C(═O)SR^{b}, —SC(═O)R^{b}, —NR^{a}R^{b}, —NR^{a}C(═O)R^{b}, —C(═O)NR^{a}R^{b}, —NR^{a}C(═O)NR^{a}R^{b}, —OC(═O)NR^{a}R^{b}, —NR^{a}C(═O)OR^{b}, —NR^{a}S(O)ₓNR^{a}R^{b}, —NR^{a}S(O)ₓR^{b} or —S(O)ₓNR^{a}R^{b}, wherein: R^{a} is H or C₁-C₁₂ alkyl; R^{b} is C₁-C₁₅ alkyl; and x is 0, 1 or 2. For example, in some embodiments R⁷ is substituted with —(C═O)OR^{b} or —O(C═O)R^{b}.

In some of the foregoing embodiments of Formula (II), R^{b} is branched C₁-C₁₆ alkyl. For example, in some embodiments R^{b} has one of the following structures:

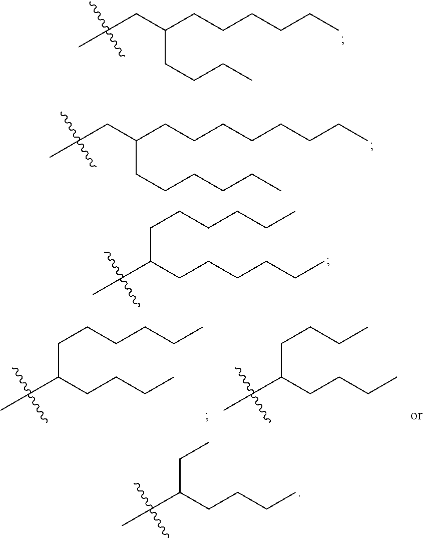

In certain other of the foregoing embodiments of Formula (II), one of R⁸ or R⁹ is methyl. In other embodiments, both R⁸ and R⁹ are methyl.

In some different embodiments of Formula (II), R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring. In some different embodiments of the foregoing, R⁸ and R⁹, together with the nitrogen atom to which they are attached, form a 6-membered heterocyclic ring, for example a piperazinyl ring.

In certain embodiments of Embodiment 3, the first and second cationic lipids are each, independently selected from a lipid of Formula II.

In still other embodiments of the foregoing lipids of Formula (II), G³ is C₂-C₄ alkylene, for example C₃ alkylene. In various different embodiments, the lipid compound has one of the structures set forth in Table 2 below

TABLE 2
Representative Lipids of Formula (II)

| No. | Structure | pKa |
|---|---|---|
| II-1 | 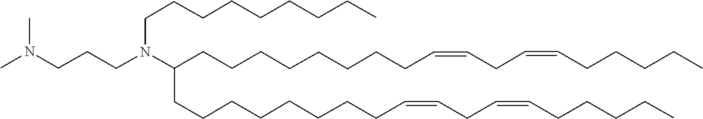 [SMILES: CCCCC/C=C\C/C=C\CCCCCCCCC(CCCCCCCC/C=C\C/C=C\CCCCC)N(CCCCCCCCC)CCCN(C)C] | 5.64 |
| II-2 | 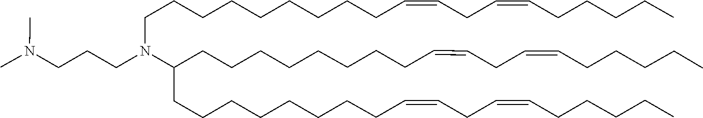 [SMILES: CCCCC/C=C\C/C=C\CCCCCCCCCN(CCCN(C)C)C(CCCCCCCC/C=C\C/C=C\CCCCC)CCCCCCCC/C=C\C/C=C\CCCCC] | — |
| II-3 | 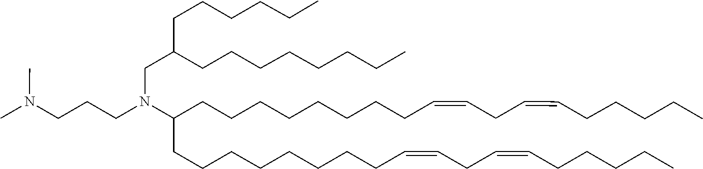 [SMILES: CCCCC/C=C\C/C=C\CCCCCCCCC(CCCCCCCC/C=C\C/C=C\CCCCC)N(CCCN(C)C)CC(CCCCCC)CCCCCCCC] | — |
| II-4 | 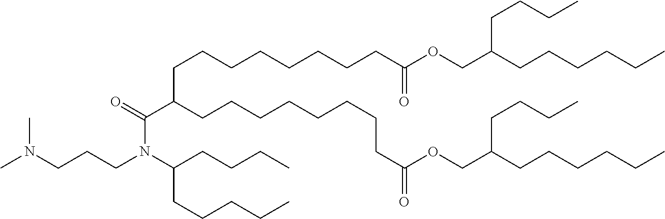 [SMILES: CCCCCCC(CCCC)COC(=O)CCCCCCCCC(CCCCCCCCC(=O)OCC(CCCC)CCCCCC)C(=O)N(CCCN(C)C)C(CCCC)CCCCC] | — |
| II-5 | 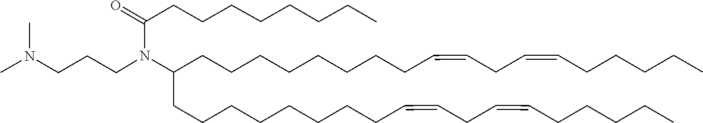 [SMILES: CCCCC/C=C\C/C=C\CCCCCCCCC(CCCCCCCC/C=C\C/C=C\CCCCC)N(CCCN(C)C)C(=O)CCCCCCCC] | 6.27 |
| II-6 | 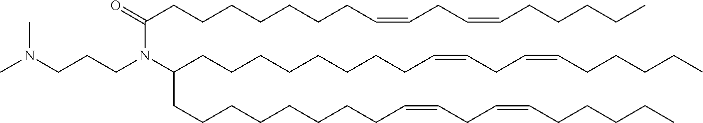 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC] | 6.14 |
| II-7 | 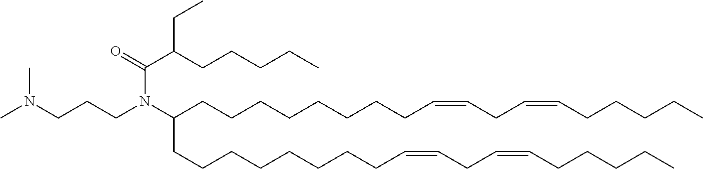 [SMILES: CCCCC/C=C\C/C=C\CCCCCCCCC(CCCCCCCC/C=C\C/C=C\CCCCC)N(CCCN(C)C)C(=O)C(CC)CCCCC] | 5.93 |
| II-8 | 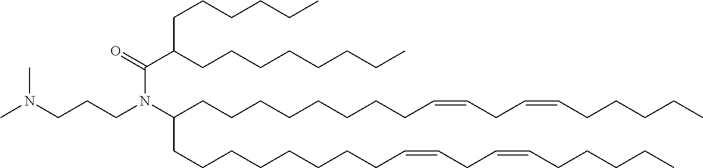 [SMILES: CCCCC/C=C\C/C=C\CCCCCCCCC(CCCCCCCC/C=C\C/C=C\CCCCC)N(CCCN(C)C)C(=O)C(CCCCCC)CCCCCCCC] | 5.35 |
| II-9 | 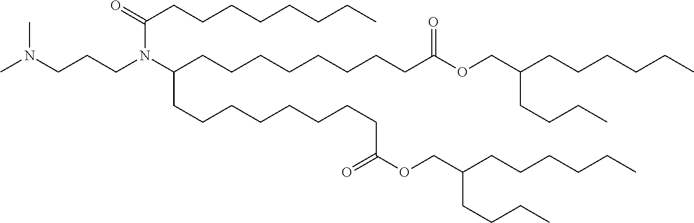 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] | 6.27 |
| II-10 | 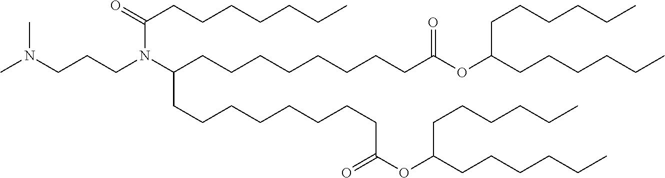 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] | 6.16 |
| II-11 | 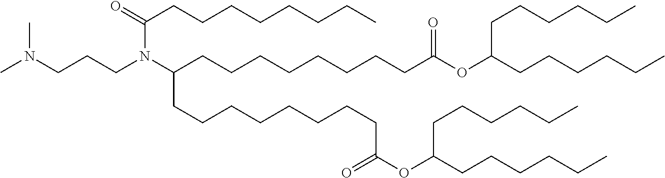 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC] | 6.13 |
| II-12 | 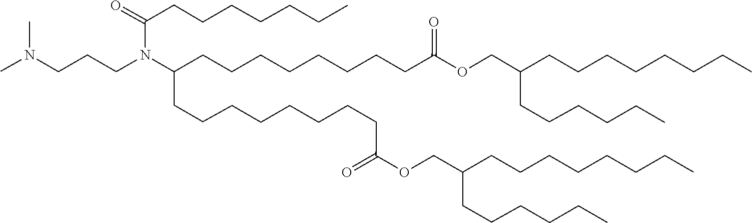 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC] | 6.21 |
| II-13 | 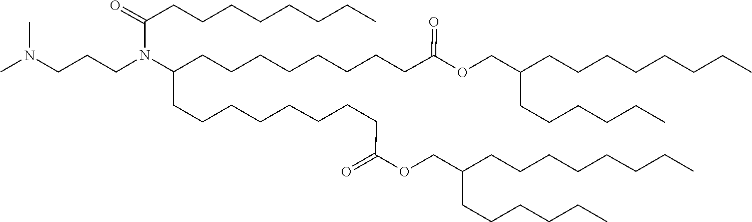 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC] | 6.22 |
| II-14 | 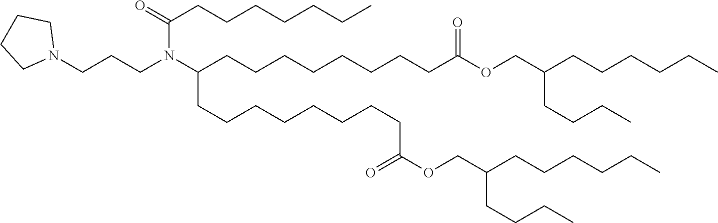 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC] | 6.33 |
| II-15 | 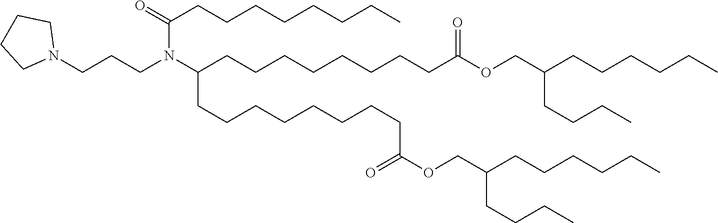 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC] | 6.32 |
| II-16 | 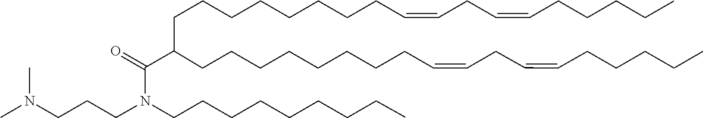 [SMILES: CCCCC/C=C\C/C=C\CCCCCCCCC(CCCCCCCC/C=C\C/C=C\CCCCC)C(=O)N(CCCCCCCCC)CCCN(C)C] | 6.37 |
| II-17 | 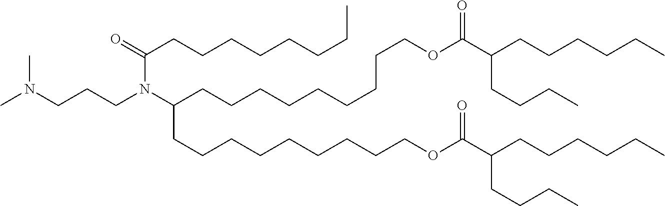 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC] | 6.27 |
| II-18 | 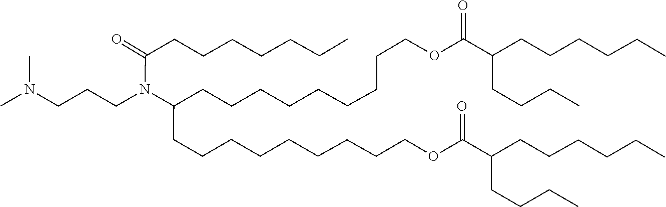 [SMILES: CCCCCCCC(=O)N(CCCN(C)C)C(CCCCCCCCCOC(=O)C(CCCC)CCCCCC)CCCCCCCCCOC(=O)C(CCCC)CCCCCC] | — |
| II-19 | 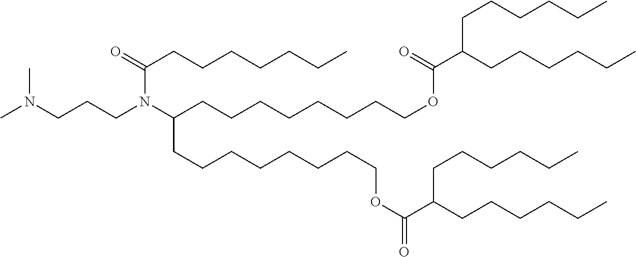 [SMILES: CCCCCCCC(=O)N(CCCN(C)C)C(CCCCCCCCOC(=O)C(CCCCCC)CCCCCC)CCCCCCCCOC(=O)C(CCCCCC)CCCCCC] | — |
| II-20 | 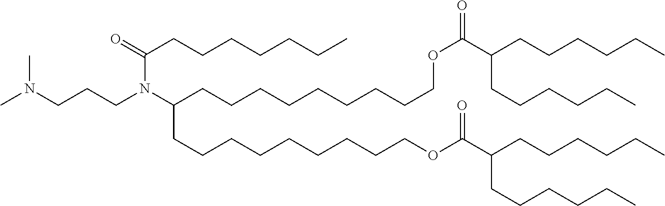 [SMILES: CCCCCCCC(=O)N(CCCN(C)C)C(CCCCCCCCCOC(=O)C(CCCCCC)CCCCCC)CCCCCCCCCOC(=O)C(CCCCCC)CCCCCC] | — |
| II-21 | 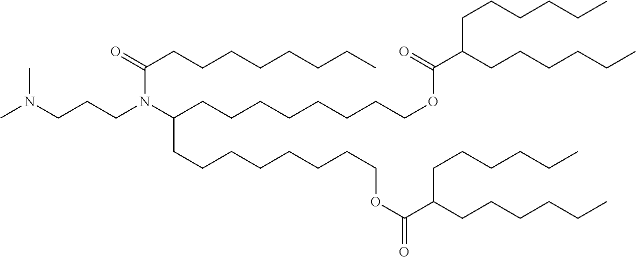 [SMILES: CCCCCCCCC(=O)N(CCCN(C)C)C(CCCCCCCCOC(=O)C(CCCCCC)CCCCCC)CCCCCCCCOC(=O)C(CCCCCC)CCCCCC] | — |
| II-22 | 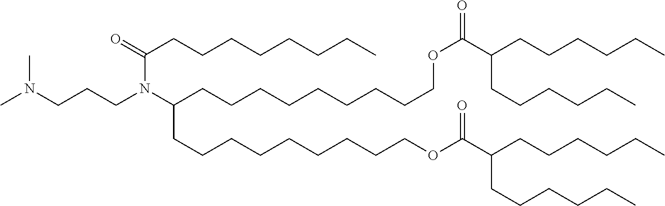 [SMILES: CCCCCCCCC(=O)N(CCCN(C)C)C(CCCCCCCCCOC(=O)C(CCCCCC)CCCCCC)CCCCCCCCCOC(=O)C(CCCCCC)CCCCCC] | — |
| II-23 | 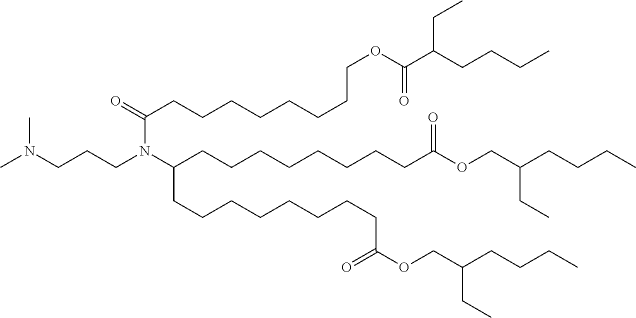 [SMILES: CCCCC(CC)COC(=O)CCCCCCCCC(CCCCCCCCC(=O)OCC(CC)CCCC)N(CCCN(C)C)C(=O)CCCCCCCCOC(=O)C(CC)CCCC] | — |
| II-24 | 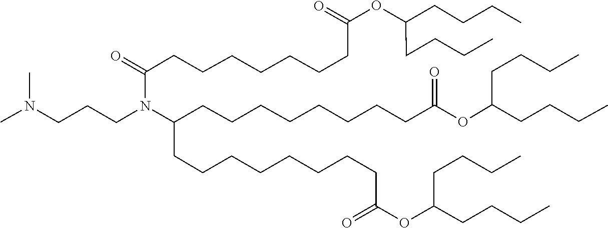 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC] | 6.14 |
| II-25 | 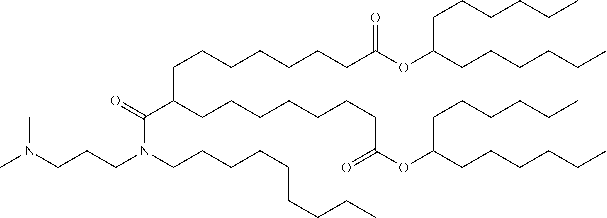 [SMILES: CCCCCCCCCN(CCCN(C)C)C(=O)C(CCCCCCCC(=O)OC(CCCCCC)CCCCCC)CCCCCCCC(=O)OC(CCCCCC)CCCCCC] | — |
| II-26 | 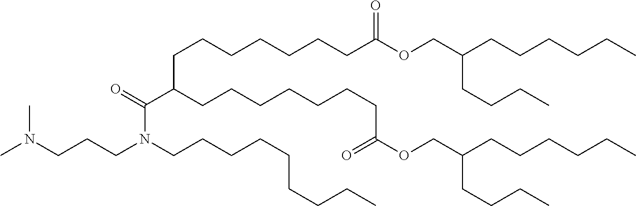 [SMILES: CCCCCCCCCN(CCCN(C)C)C(=O)C(CCCCCCCC(=O)OCC(CCCC)CCCCCC)CCCCCCCC(=O)OCC(CCCC)CCCCCC] | — |
| II-27 | 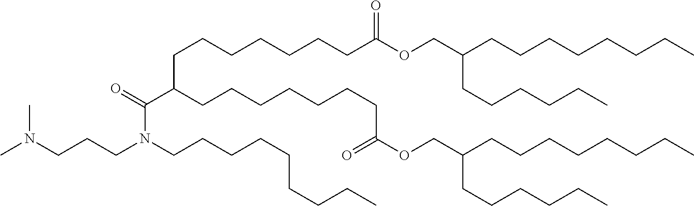 [SMILES: CCCCCCCCCN(CCCN(C)C)C(=O)C(CCCCCCCC(=O)OCC(CCCCCC)CCCCCCCC)CCCCCCCC(=O)OCC(CCCCCC)CCCCCCCC] | — |
| II-28 | 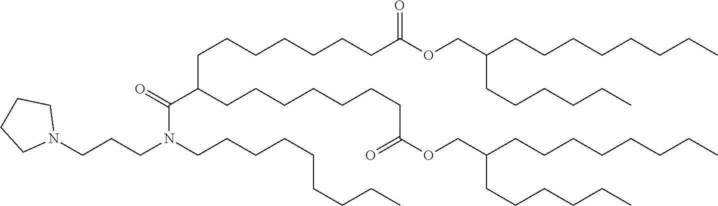 [SMILES: CCCCCCCCCN(CCCN1CCCC1)C(=O)C(CCCCCCCC(=O)OCC(CCCCCC)CCCCCCCC)CCCCCCCC(=O)OCC(CCCCCC)CCCCCCCC] | — |
| II-29 | 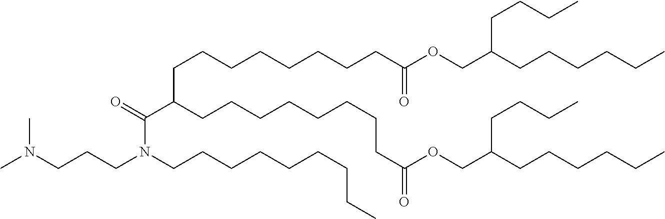 [SMILES: CCCCCCCCCN(CCCN(C)C)C(=O)C(CCCCCCCCC(=O)OCC(CCCC)CCCCCC)CCCCCCCCC(=O)OCC(CCCC)CCCCCC] | — |
| II-30 | 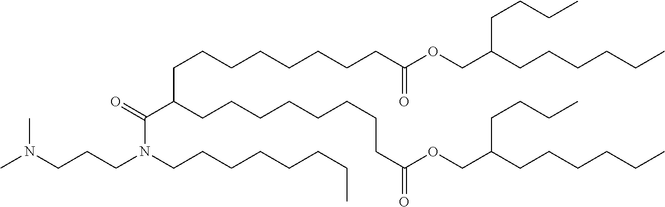 [SMILES: CCCCCCCCN(CCCN(C)C)C(=O)C(CCCCCCCCC(=O)OCC(CCCC)CCCCCC)CCCCCCCCC(=O)OCC(CCCC)CCCCCC] | — |
| II-31 | 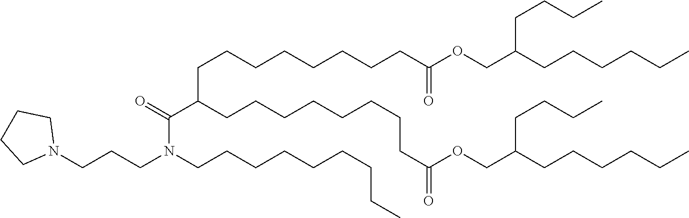 [SMILES: CCCCCCCCCN(CCCN1CCCC1)C(=O)C(CCCCCCCCC(=O)OCC(CCCC)CCCCCC)CCCCCCCCC(=O)OCC(CCCC)CCCCCC] | — |
| II-32 | 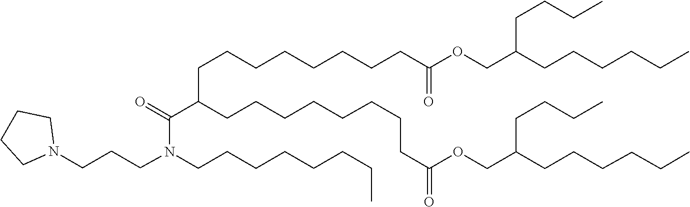 [SMILES: CCCCCCCCN(CCCN1CCCC1)C(=O)C(CCCCCCCCC(=O)OCC(CCCC)CCCCCC)CCCCCCCCC(=O)OCC(CCCC)CCCCCC] | — |
| II-33 | 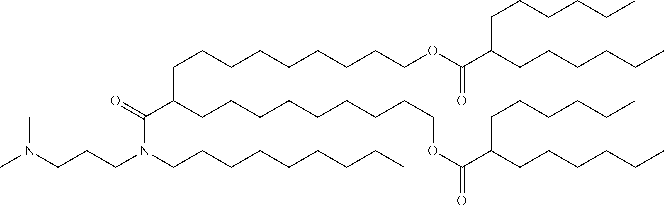 [SMILES: CCCCCCCCCN(CCCN(C)C)C(=O)C(CCCCCCCCCOC(=O)C(CCCCCC)CCCCCC)CCCCCCCCCOC(=O)C(CCCCCC)CCCCCC] | — |
| II-34 | 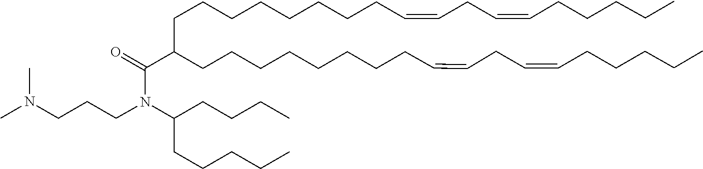 [SMILES: CCCCC/C=C\C/C=C\CCCCCCCCC(CCCCCCCC/C=C\C/C=C\CCCCC)C(=O)N(CCCN(C)C)C(CCCC)CCCCC] | — |
| II-35 | 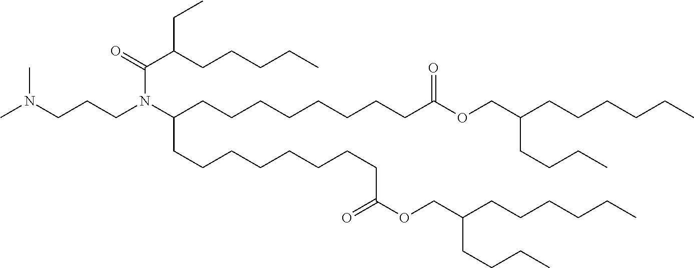 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC] | 5.97 |
| II-36 | 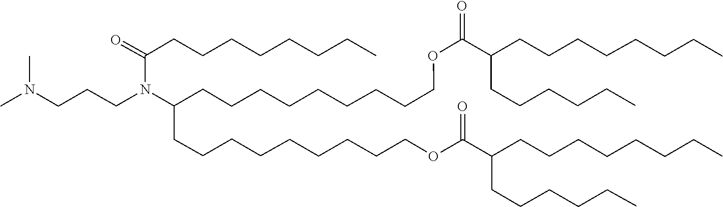 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC] | 6.13 |
| II-37 | 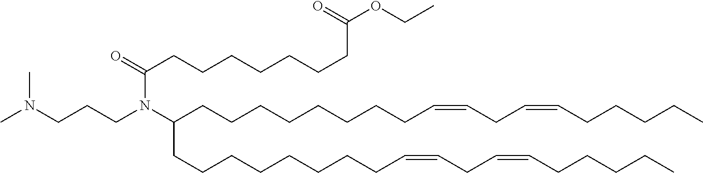 [SMILES: CCCCC/C=C\C/C=C\CCCCCCCCC(CCCCCCCC/C=C\C/C=C\CCCCC)N(CCCN(C)C)C(=O)CCCCCCCC(=O)OCC] | 5.61 |
| II-38 | 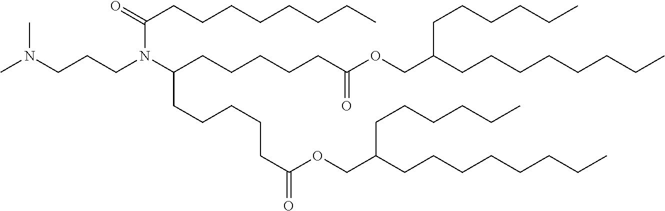 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC] | 6.45 |
| II-39 | 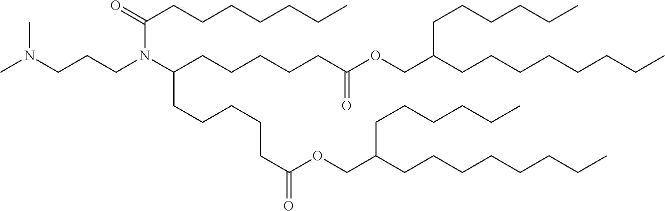 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC] | 6.45 |
| II-40 | 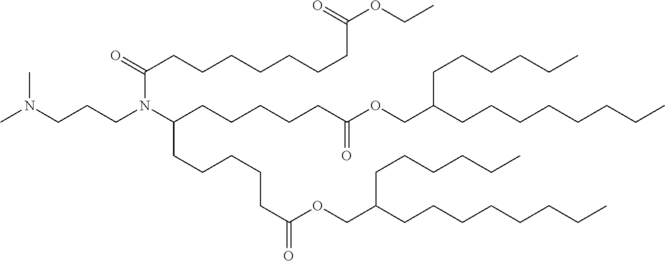 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC] | 6.57 |
| II-41 | 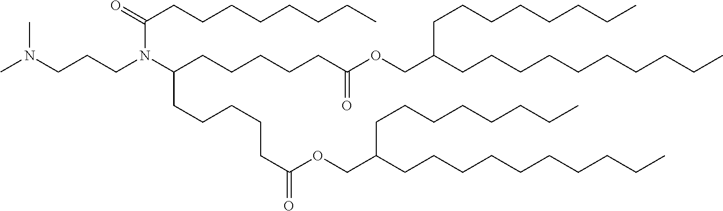 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC] | — |
| II-42 | 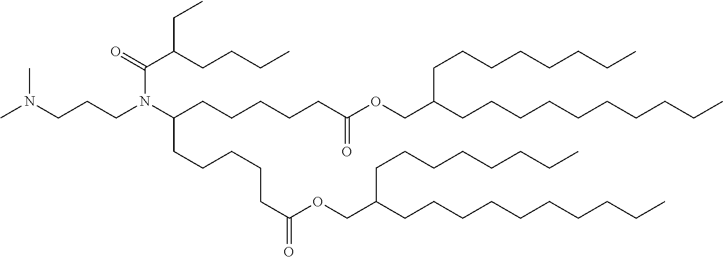 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)C(CC)CCCC] | — |
| II-43 | 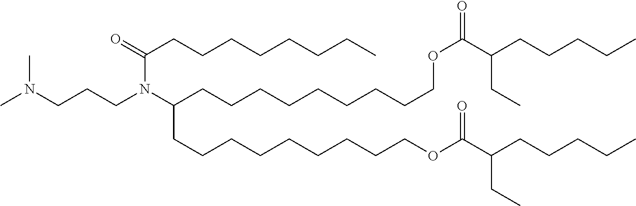 [SMILES: CCCCCCCCC(=O)N(CCCN(C)C)C(CCCCCCCCCOC(=O)C(CC)CCCCC)CCCCCCCCCOC(=O)C(CC)CCCCC] | — |
| II-44 | 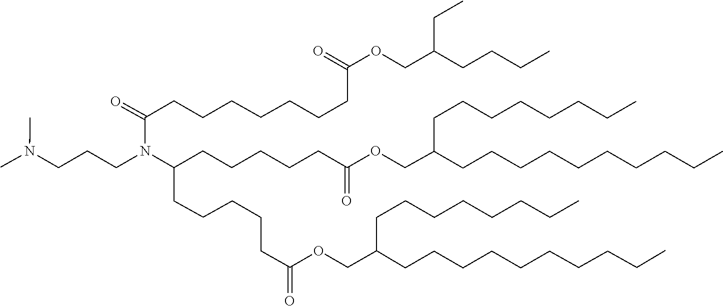 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC(=O)OCC(CC)CCCC] | — |
| II-45 | 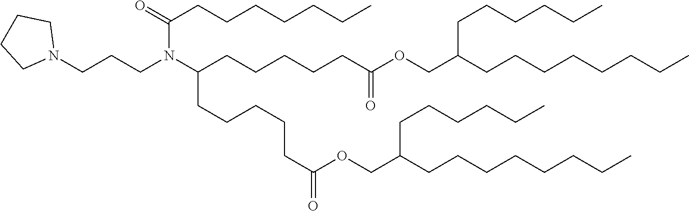 [SMILES: CCCCCCCCC(CCCCCC)COC(=O)CCCCCC(CCCCCC(=O)OCC(CCCCCC)CCCCCCCC)N(CCCN1CCCC1)C(=O)CCCCCCC] | — |
| II-46 | 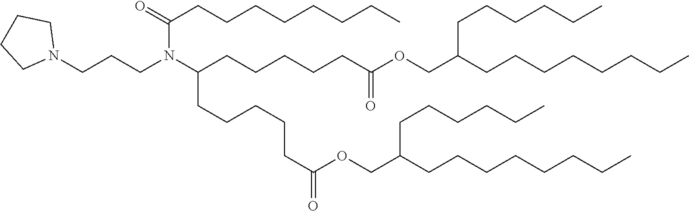 [SMILES: CCCCCCCCC(=O)N(CCCN1CCCC1)C(CCCCCC(=O)OCC(CCCCCC)CCCCCCCC)CCCCCC(=O)OCC(CCCCCC)CCCCCCCC] | — |

In some other embodiments, the cationic lipid of Embodiments 1, 2, 3, 4 or 5 has a structure of Formula III:

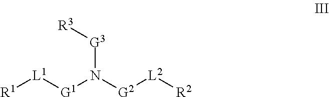

or a pharmaceutically acceptable salt, prodrug or stereoisomer thereof, wherein:

one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or

—NR^{a}C(═O)O— or a direct bond;

G¹ and G² are each independently unsubstituted C₁-C₁₂ alkylene or C₁-C₁₂ alkenylene;

G³ is C₁-C₂₄ alkylene, C₁-C₂₄ alkenylene, C₃-C₈ cycloalkylene, C₃-C₈ cycloalkenylene;

R^{a} is H or C₁-C₁₂ alkyl;

R¹ and R² are each independently C₆-C₂₄ alkyl or C₆-C₂₄ alkenyl;

R³ is H, OR⁵, CN, —C(═O)OR⁴, —OC(═O)R⁴ or —NR⁵C(═O)R⁴;

R⁴ is C₁-C₁₂ alkyl;

R⁵ is H or C₁-C₆ alkyl; and

x is 0, 1 or 2.

In some of the foregoing embodiments of Formula (III), the lipid has one of the following Formulae (IIIA) or (IIIB)

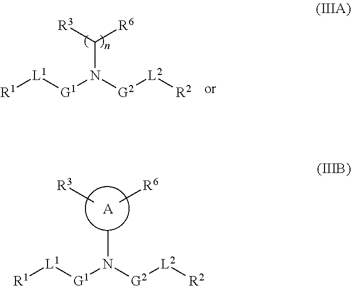

wherein:

A is a 3 to 8-membered cycloalkyl or cycloalkylene ring;

R⁶ is, at each occurrence, independently H, OH or C₁-C₂₄ alkyl;

n is an integer ranging from 1 to 15.

In some of the foregoing embodiments of Formula (III), the lipid has Formula (IIIA), and in other embodiments, the lipid has Formula

In other embodiments of Formula (III), the lipid has one of the following Formulae (IIIC) or (IIID):

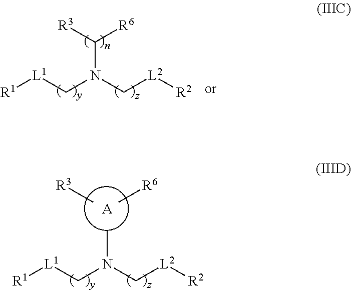

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following Formulae (IIIE) or (IIIF):

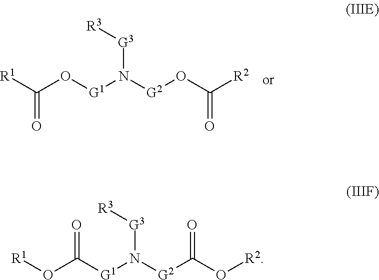

In some of the foregoing embodiments of Formula (III), the lipid has one of the following Formulae (IIIG), (IIIH), (IIII), or (IIID):

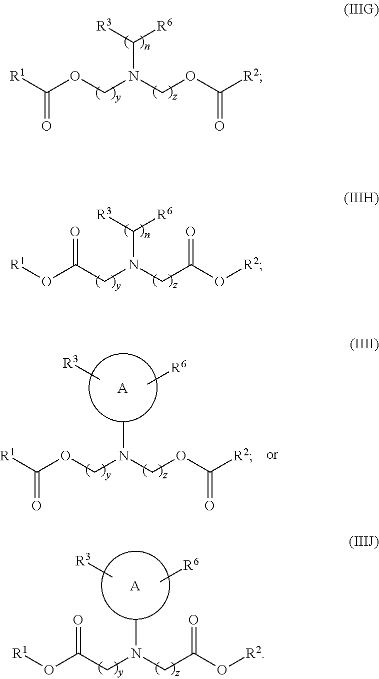

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G3 is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

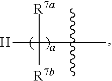

wherein:

R^{7a} and R^{7b} are, at each occurrence, independently H or C₁-C₁₂ alkyl; and

a is an integer from 2 to 12,

wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

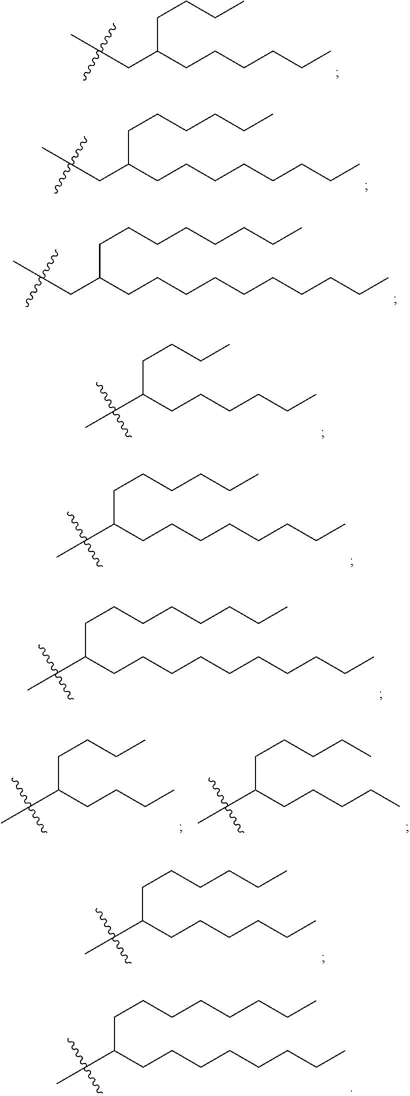

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In some specific embodiments of Embodiment 3, the first and second cationic lipids are each, independently selected from a lipid of Formula III.

In various different embodiments, a cationic lipid of any one of the disclosed embodiments (e.g., the cationic lipid, the first cationic lipid, the second cationic lipid) of Formula (III) has one of the structures set forth in Table 3 below.

TABLE 3
Representative Compounds of Formula (III)

| No. | Structure | pKa |
|---|---|---|
| III-1 | 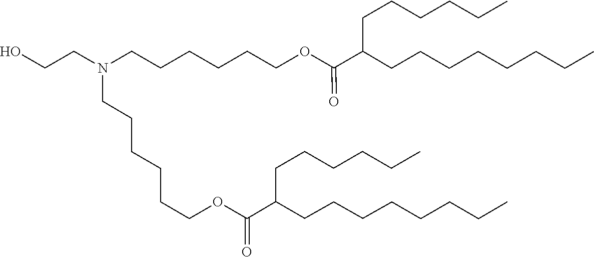 [SMILES: CCCCCCCCC(CCCCCC)C(=O)OCCCCCCN(CCO)CCCCCCOC(=O)C(CCCCCC)CCCCCCCC] | 5.89 |
| III-2 | 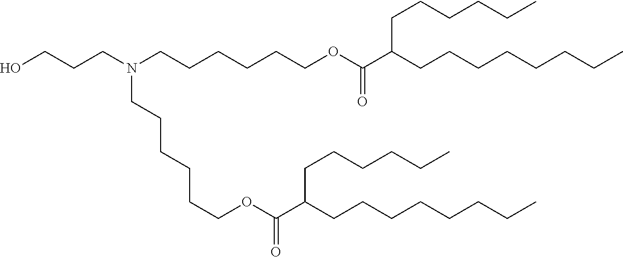 [SMILES: CCCCCCCCC(CCCCCC)C(=O)OCCCCCCN(CCCO)CCCCCCOC(=O)C(CCCCCC)CCCCCCCC] | 6.05 |
| III-3 | 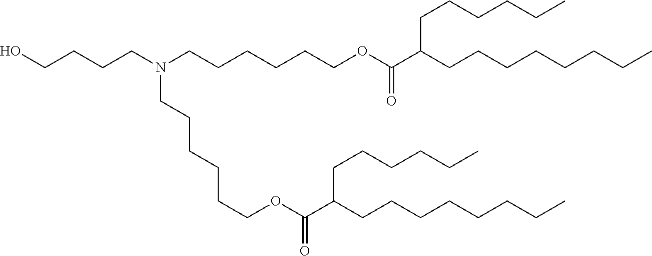 [SMILES: CCCCCCCCC(CCCCCC)C(=O)OCCCCCCN(CCCCO)CCCCCCOC(=O)C(CCCCCC)CCCCCCCC] | 6.09 |
| III-4 | 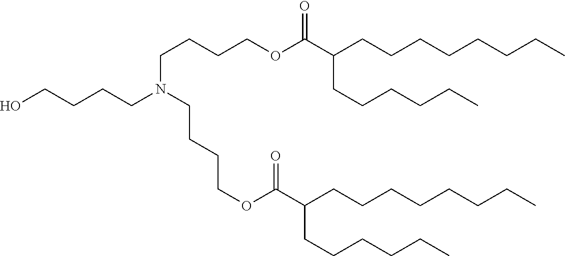 [SMILES: CCCCCCCCC(CCCCCC)C(=O)OCCCCN(CCCCO)CCCCOC(=O)C(CCCCCC)CCCCCCCC] | 5.60 |
| III-5 | 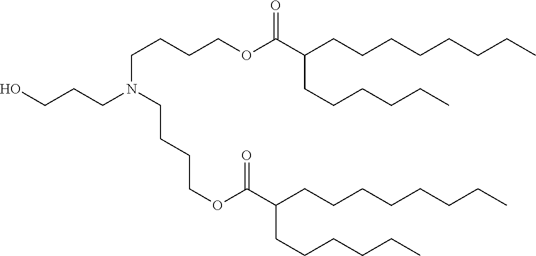 [SMILES: CCCCCCCCC(CCCCCC)C(=O)OCCCCN(CCCO)CCCCOC(=O)C(CCCCCC)CCCCCCCC] | 5.59 |
| III-6 | 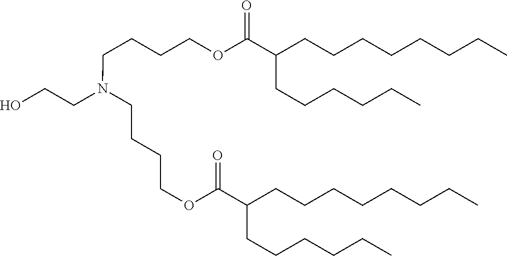 [SMILES: CCCCCCCCC(CCCCCC)C(=O)OCCCCN(CCO)CCCCOC(=O)C(CCCCCC)CCCCCCCC] | 5.42 |
| III-7 | 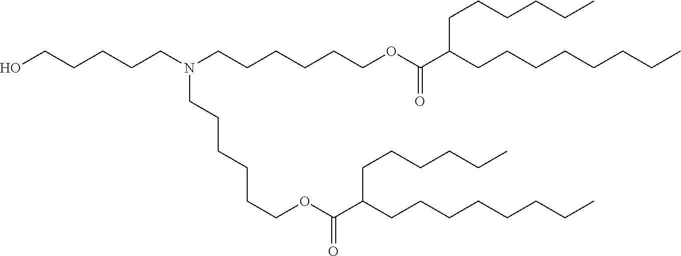 [SMILES: CCCCCCCCC(CCCCCC)C(=O)OCCCCCCN(CCCCCO)CCCCCCOC(=O)C(CCCCCC)CCCCCCCC] | 6.11 |
| III-8 | 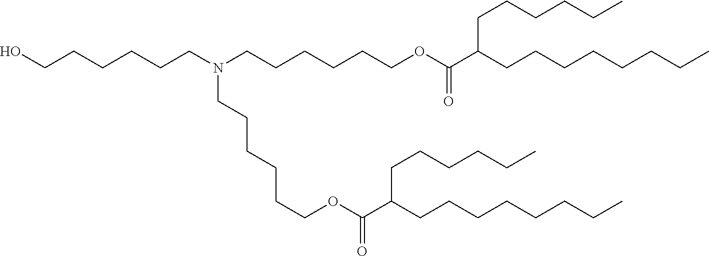 [SMILES: CCCCCCCCC(CCCCCC)C(=O)OCCCCCCN(CCCCCCO)CCCCCCOC(=O)C(CCCCCC)CCCCCCCC] | 5.84 |
| III-9 | 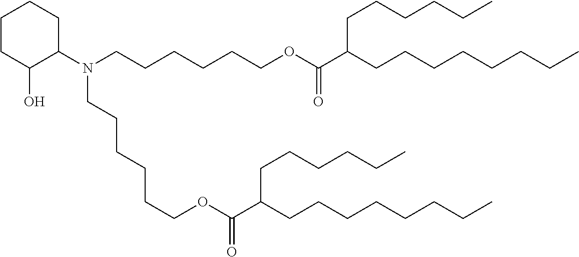 [SMILES: CCCCCCCCC(CCCCCC)C(=O)OCCCCCCN(CCCCCCOC(=O)C(CCCCCC)CCCCCCCC)C1CCCCC1O] | — |
| III-10 | 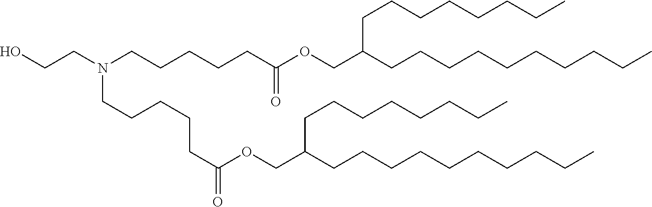 [SMILES: CCCCCCCCCCC(CCCCCCCC)COC(=O)CCCCCN(CCO)CCCCCC(=O)OCC(CCCCCCCC)CCCCCCCCCC] | — |
| III-11 | 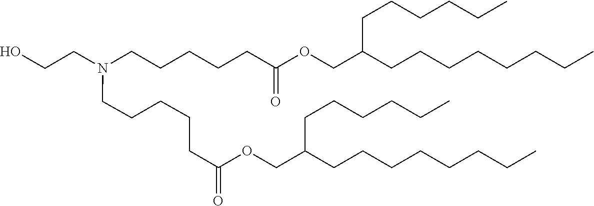 [SMILES: CCCCCCCCC(CCCCCC)COC(=O)CCCCCN(CCO)CCCCCC(=O)OCC(CCCCCC)CCCCCCCC] | — |
| III-12 | 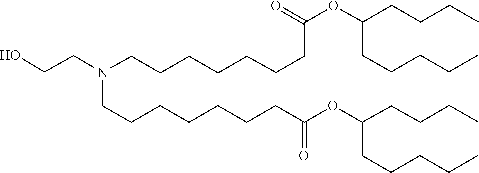 [SMILES: CCCCCC(CCCC)OC(=O)CCCCCCCN(CCO)CCCCCCCC(=O)OC(CCCC)CCCCC] | — |
| III-13 | 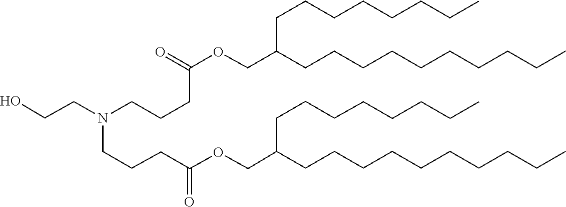 [SMILES: CCCCCCCCCCC(CCCCCCCC)COC(=O)CCCN(CCO)CCCC(=O)OCC(CCCCCCCC)CCCCCCCCCC] | — |
| III-14 | 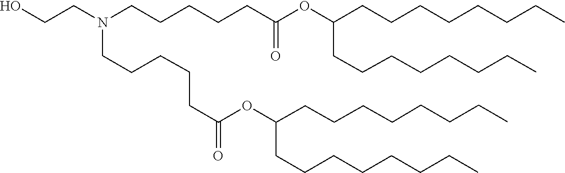 [SMILES: CCCCCCCCC(CCCCCCCC)OC(=O)CCCCCN(CCO)CCCCCC(=O)OC(CCCCCCCC)CCCCCCCC] | — |
| III-15 | 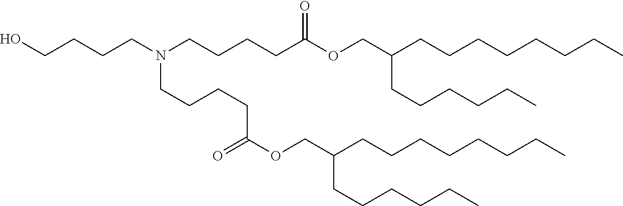 [SMILES: CCCCCCCCC(CCCCCC)COC(=O)CCCCN(CCCCO)CCCCC(=O)OCC(CCCCCC)CCCCCCCC] | 6.14 |
| III-16 | 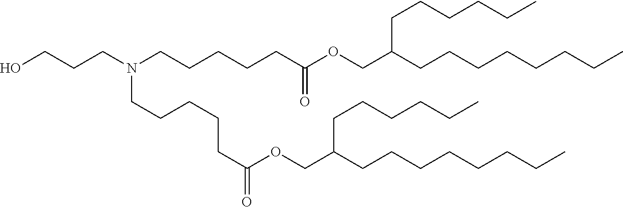 [SMILES: CCCCCCCCC(CCCCCC)COC(=O)CCCCCN(CCCO)CCCCCC(=O)OCC(CCCCCC)CCCCCCCC] | 6.31 |
| III-17 | 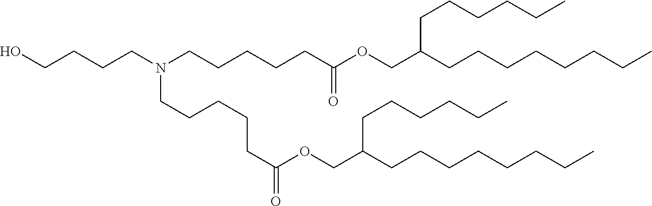 [SMILES: CCCCCCCCC(CCCCCC)COC(=O)CCCCCN(CCCCO)CCCCCC(=O)OCC(CCCCCC)CCCCCCCC] | 6.28 |
| III-18 | 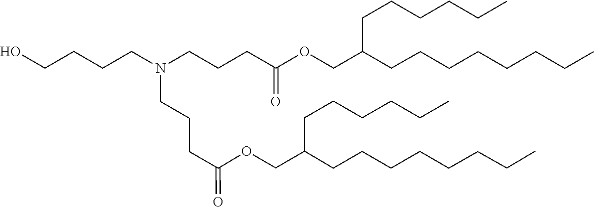 [SMILES: CCCCCCCCC(CCCCCC)COC(=O)CCCN(CCCCO)CCCC(=O)OCC(CCCCCC)CCCCCCCC] | — |
| III-19 | 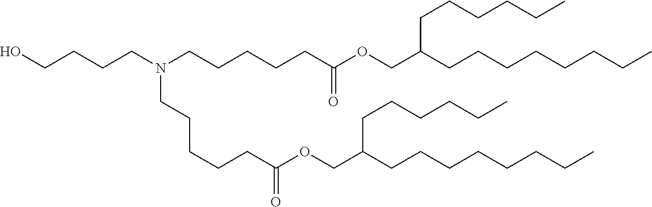 [SMILES: CCCCCCCCC(CCCCCC)COC(=O)CCCCCN(CCCCO)CCCCCC(=O)OCC(CCCCCC)CCCCCCCC] | — |
| III-20 | 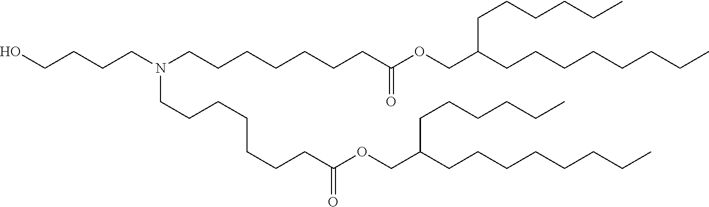 [SMILES: CCCCCCCCC(CCCCCC)COC(=O)CCCCCCCN(CCCCO)CCCCCCCC(=O)OCC(CCCCCC)CCCCCCCC] | 6.36 |
| III-21 | 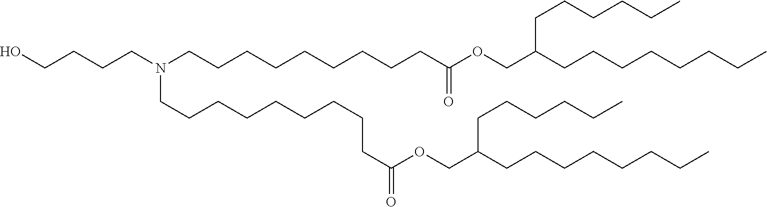 [SMILES: CCCCCCCCC(CCCCCC)COC(=O)CCCCCCCCCN(CCCCO)CCCCCCCCCC(=O)OCC(CCCCCC)CCCCCCCC] | — |
| III-22 | 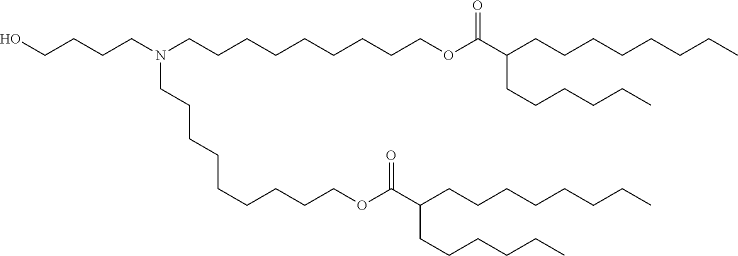 [SMILES: CCCCCCCCC(CCCCCC)C(=O)OCCCCCCCCCN(CCCCO)CCCCCCCCCOC(=O)C(CCCCCC)CCCCCCCC] | 6.10 |
| III-23 | 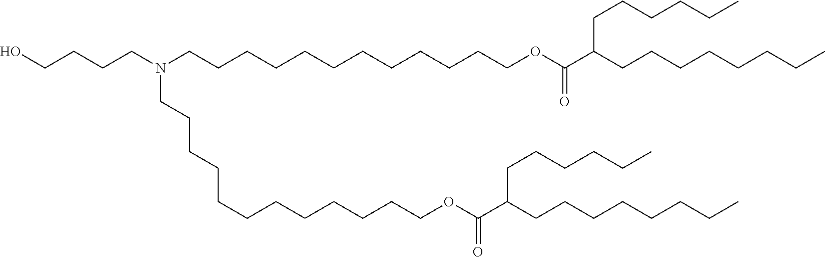 [SMILES: CCCCCCCCC(CCCCCC)C(=O)OCCCCCCCCCCCCN(CCCCO)CCCCCCCCCCCCOC(=O)C(CCCCCC)CCCCCCCC] | 5.98 |
| III-24 | 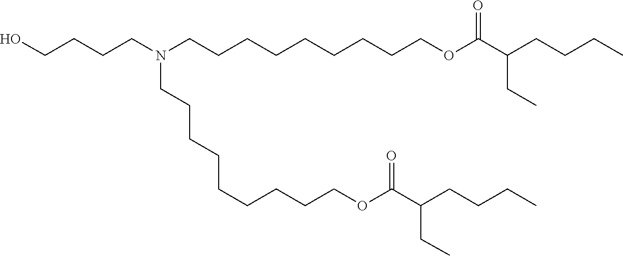 [SMILES: CCCCC(CC)C(=O)OCCCCCCCCCN(CCCCO)CCCCCCCCCOC(=O)C(CC)CCCC] | — |
| III-25 | 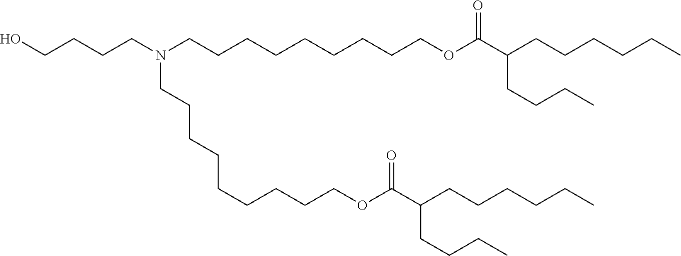 [SMILES: CCCCCCC(CCCC)C(=O)OCCCCCCCCCN(CCCCO)CCCCCCCCCOC(=O)C(CCCC)CCCCCC] | 6.22 |
| III-26 | 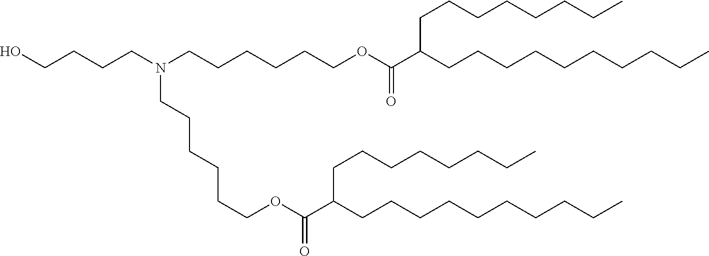 [SMILES: CCCCCCCCCCC(CCCCCCCC)C(=O)OCCCCCCN(CCCCO)CCCCCCOC(=O)C(CCCCCCCC)CCCCCCCCCC] | 5.84 |
| III-27 | 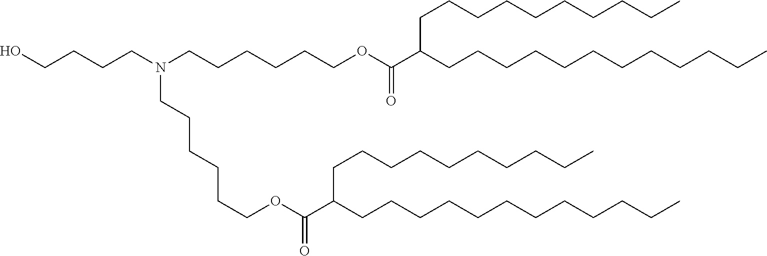 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] | 5.77 |
| III-28 | 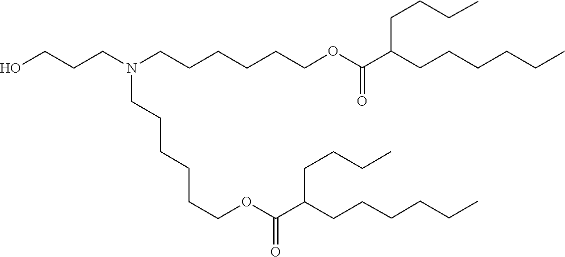 [SMILES: CCCCCCC(CCCC)C(=O)OCCCCCCN(CCCO)CCCCCCOC(=O)C(CCCC)CCCCCC] | — |
| III-29 | 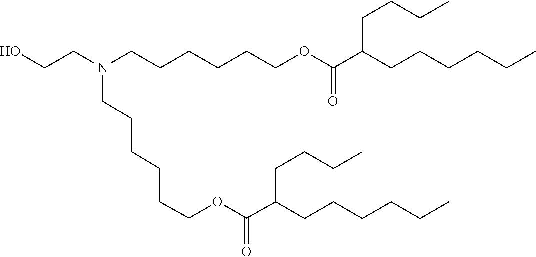 [SMILES: CCCCCCC(CCCC)C(=O)OCCCCCCN(CCO)CCCCCCOC(=O)C(CCCC)CCCCCC] | — |
| III-30 | 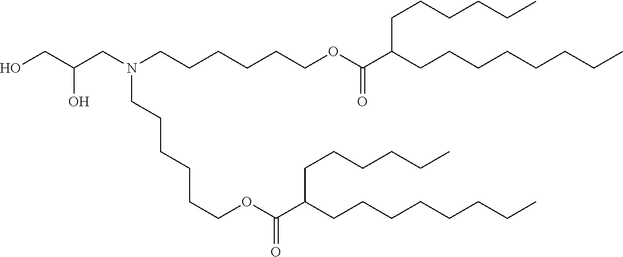 [SMILES: CCCCCCCCC(CCCCCC)C(=O)OCCCCCCN(CCCCCCOC(=O)C(CCCCCC)CCCCCCCC)CC(O)CO] | 6.09 |
| III-31 | 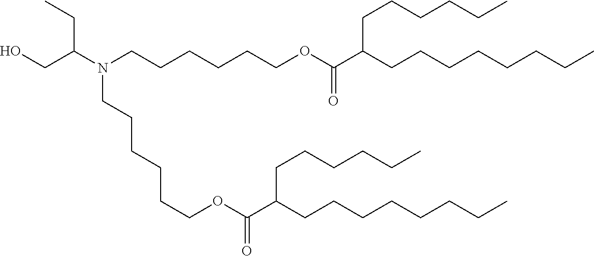 [SMILES: CCCCCCCCC(CCCCCC)C(=O)OCCCCCCN(CCCCCCOC(=O)C(CCCCCC)CCCCCCCC)C(CC)CO] | — |
| III-32 | 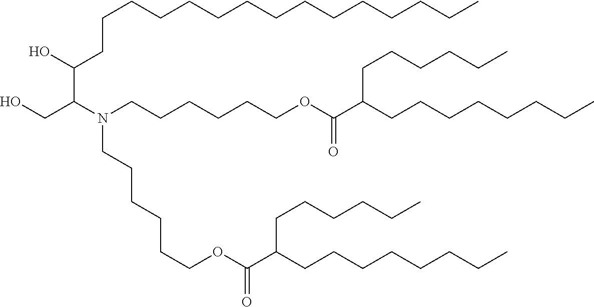 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] | — |
| III-33 | 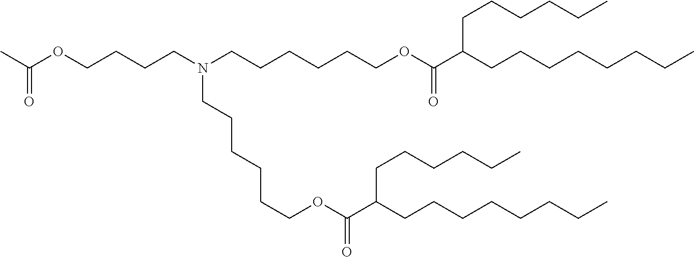 [SMILES: CCCCCCCCC(CCCCCC)C(=O)OCCCCCCN(CCCCCCOC(=O)C(CCCCCC)CCCCCCCC)CCCCOC(C)=O] | — |
| III-34 | 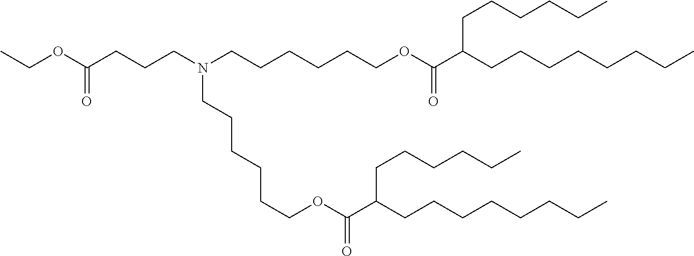 [SMILES: CCCCCCCCC(CCCCCC)C(=O)OCCCCCCN(CCCCCCOC(=O)C(CCCCCC)CCCCCCCC)CCCC(=O)OCC] | — |
| III-35 | 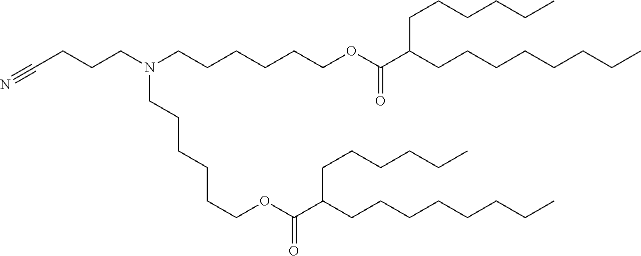 [SMILES: CCCCCCCCC(CCCCCC)C(=O)OCCCCCCN(CCCC#N)CCCCCCOC(=O)C(CCCCCC)CCCCCCCC] | — |
| III-36 | 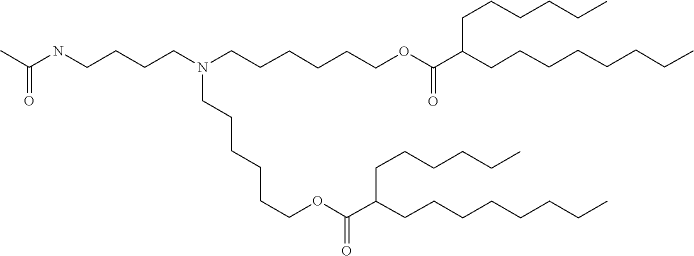 | — |
| III-37 | 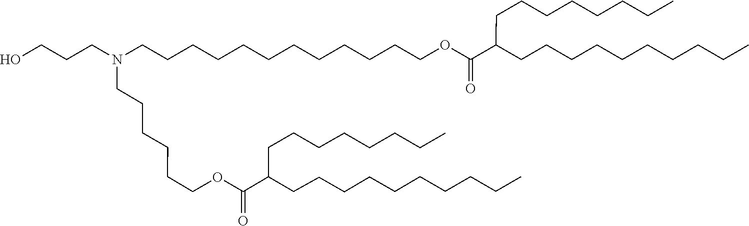 [SMILES: CCCCCCCCCCC(CCCCCCCC)C(=O)OCCCCCCCCCCCCN(CCCO)CCCCCCOC(=O)C(CCCCCCCC)CCCCCCCCCC] | — |
| III-38 | 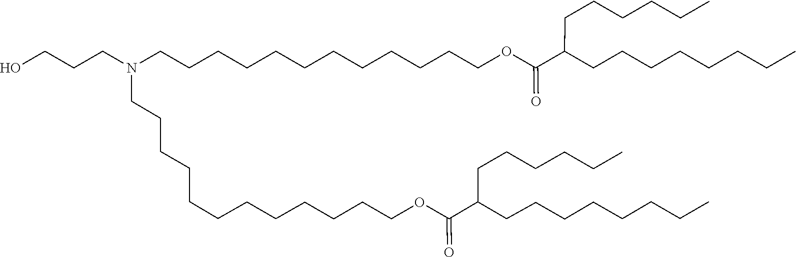 [SMILES: CCCCCCCCC(CCCCCC)C(=O)OCCCCCCCCCCCCN(CCCO)CCCCCCCCCCCCOC(=O)C(CCCCCC)CCCCCCCC] | — |
| III-39 | 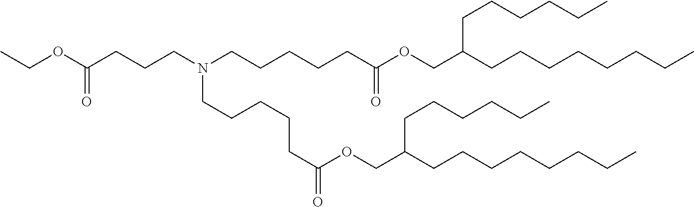 [SMILES: CCCCCCCCC(CCCCCC)COC(=O)CCCCCN(CCCCCC(=O)OCC(CCCCCC)CCCCCCCC)CCCC(=O)OCC] | — |
| III-40 | 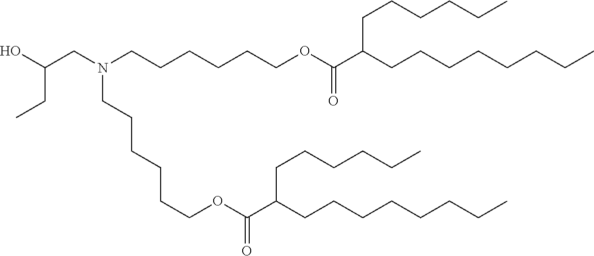 [SMILES: CCCCCCCCC(CCCCCC)C(=O)OCCCCCCN(CCCCCCOC(=O)C(CCCCCC)CCCCCCCC)CC(O)CC] | — |
| III-41 | 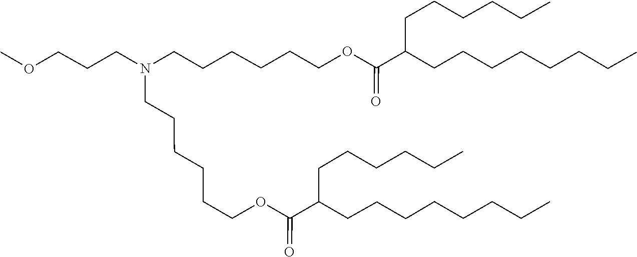 [SMILES: CCCCCCCCC(CCCCCC)C(=O)OCCCCCCN(CCCCCCOC(=O)C(CCCCCC)CCCCCCCC)CCCOC] | — |
| III-42 | 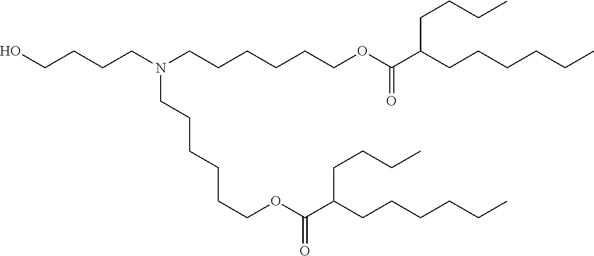 [SMILES: CCCCCCC(CCCC)C(=O)OCCCCCCN(CCCCO)CCCCCCOC(=O)C(CCCC)CCCCCC] | — |
| III-43 | 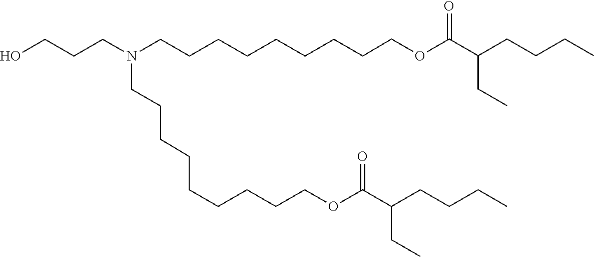 [SMILES: CCCCC(CC)C(=O)OCCCCCCCCCN(CCCO)CCCCCCCCCOC(=O)C(CC)CCCC] | — |
| III-44 | 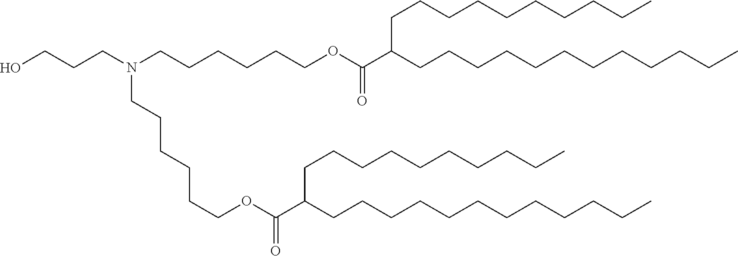 [SMILES: CCCCCCCCCCCCC(CCCCCCCCCC)C(=O)OCCCCCCN(CCCO)CCCCCCOC(=O)C(CCCCCCCCCC)CCCCCCCCCCCC] | — |
| III-45 | 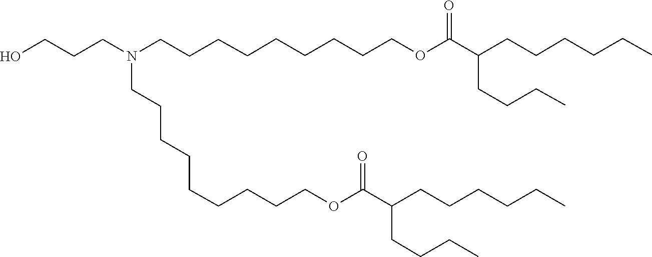 [SMILES: CCCCCCC(CCCC)C(=O)OCCCCCCCCCN(CCCO)CCCCCCCCCOC(=O)C(CCCC)CCCCCC] | — |
| III-46 | 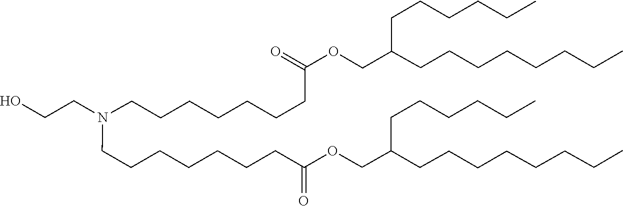 [SMILES: CCCCCCCCC(CCCCCC)COC(=O)CCCCCCCN(CCO)CCCCCCCC(=O)OCC(CCCCCC)CCCCCCCC] | — |
| III-47 | 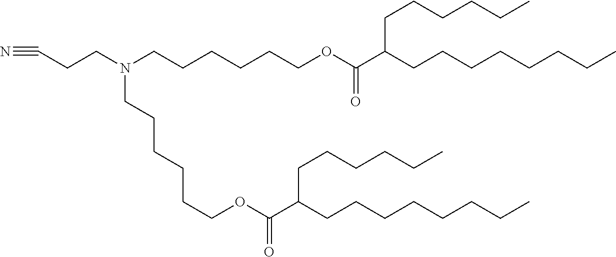 [SMILES: CCCCCCCCC(CCCCCC)C(=O)OCCCCCCN(CCC#N)CCCCCCOC(=O)C(CCCCCC)CCCCCCCC] | — |
| III-48 | 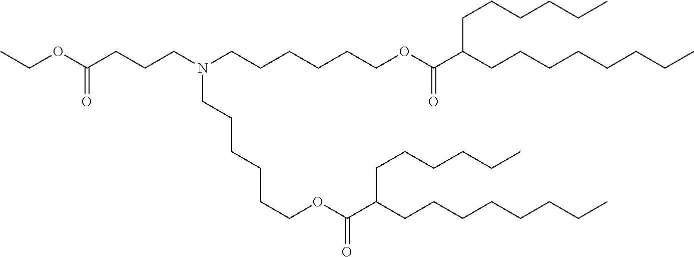 [SMILES: CCCCCCCCC(CCCCCC)C(=O)OCCCCCCN(CCCCCCOC(=O)C(CCCCCC)CCCCCCCC)CCCC(=O)OCC] | — |
| III-49 | 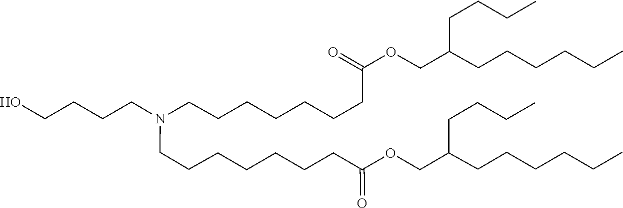 [SMILES: CCCCCCC(CCCC)COC(=O)CCCCCCCN(CCCCO)CCCCCCCC(=O)OCC(CCCC)CCCCCC] | — |

In one embodiment, the cationic lipid of any one of Embodiments 1, 2, 3, 4 or 5 has a structure of Formula (IV):

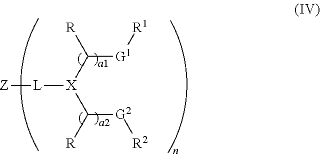

or a pharmaceutically acceptable salt, prodrug or stereoisomer thereof, wherein:

one of G¹ or G² is, at each occurrence, —O(C═O)—, —(C═O)O—, —C(═O)—, —O—, —S(O)_{y}, —S—S—, —C(═O)S—, SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O—, and the other of G¹ or G² is, at each occurrence, —O(C═O)—, —(C═O)O—, —C(═O)—, —O—, —S(O)_{y}—, —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;

L is, at each occurrence, ˜O(C═O)—, wherein ˜ represents a covalent bond to X;

X is CR^{a};

Z is alkyl, cycloalkyl or a monovalent moiety comprising at least one polar functional group when n is 1; or Z is alkylene, cycloalkylene or a polyvalent moiety comprising at least one polar functional group when n is greater than 1;

R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;

R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;

R¹ and R² have, at each occurrence, the following structure, respectively:

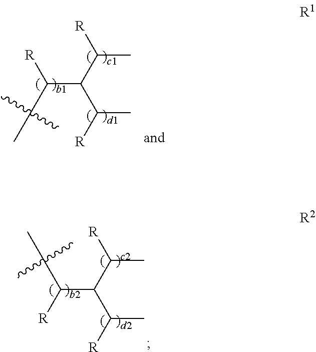

a¹ and a² are, at each occurrence, independently an integer from 3 to 12;

b¹ and b² are, at each occurrence, independently 0 or 1;

c¹ and c² are, at each occurrence, independently an integer from 5 to 10;

d¹ and d² are, at each occurrence, independently an integer from 5 to 10;

y is, at each occurrence, independently an integer from 0 to 2; and

n is an integer from 1 to 6,

wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In some embodiments of Formula (IV), G¹ and G² are each independently

### —O(C═O)— or —(C═O)O—.

In other embodiments of Formula (IV), X is CH.

In different embodiments of Formula (IV), the sum of a¹+b¹+c¹ or the sum of a²+b²+c² is an integer from 12 to 26.

In still other embodiments of Formula (IV), a¹ and a² are independently an integer from 3 to 10. For example, in some embodiments a¹ and a² are independently an integer from 4 to 9.

In various embodiments of Formula (IV), b¹ and b² are 0. In different embodiments, b¹ and b² are 1.

In more embodiments of Formula (IV), c¹, c², d¹ and d² are independently an integer from 6 to 8.

In other embodiments of Formula (IV), c¹ and c² are, at each occurrence, independently an integer from 6 to 10, and d¹ and d² are, at each occurrence, independently an integer from 6 to 10.

In other embodiments of Formula (IV), c¹ and c² are, at each occurrence, independently an integer from 5 to 9, and d¹ and d² are, at each occurrence, independently an integer from 5 to 9.

In more embodiments of Formula (IV), Z is alkyl, cycloalkyl or a monovalent moiety comprising at least one polar functional group when n is 1. In other embodiments, Z is alkyl.

In various embodiments of the foregoing Formula (IV), R is, at each occurrence, independently either: (a) H or methyl; or (b) R together with the carbon atom to which it is bound is taken together with an adjacent R and the carbon atom to which it is bound to form a carbon-carbon double bond. In certain embodiments, each R is H. In other embodiments at least one R together with the carbon atom to which it is bound is taken together with an adjacent R and the carbon atom to which it is bound to form a carbon-carbon double bond.

In other embodiments of the compound of Formula (IV), R¹ and R² independently have one of the following structures:

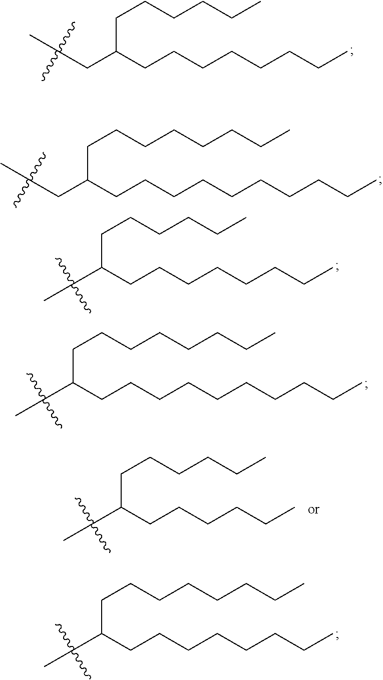

In certain embodiments of Formula (IV), the compound has one of the following structures:

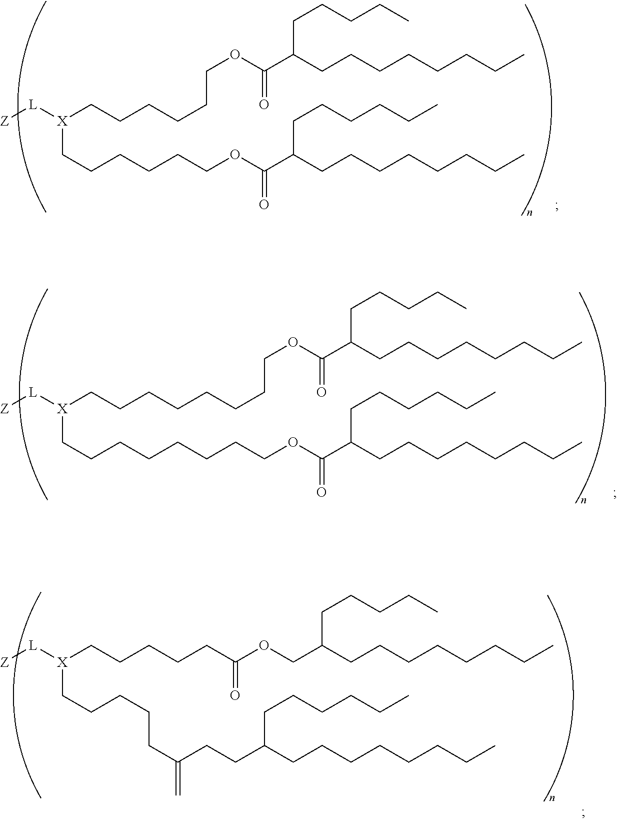

In still different embodiments the cationic lipid of Embodiments 1, 2, 3, 4 or 5 has the structure of Formula (V):

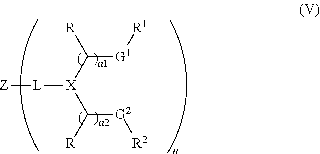

or a pharmaceutically acceptable salt, prodrug or stereoisomer thereof, wherein:

one of G¹ or G² is, at each occurrence, —O(C═O)—, —(C═O)O—, —C(═O)—, —O—, —S(O)_{y}—, —S—S—, —C(═O)S—, SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O—, and the other of G¹ or G² is, at each occurrence, —O(C═O)—, —(C═O)O—, —C(═O)—, —O—, —S(O)_{y}—, —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;

L is, at each occurrence, ˜O(C═O)—, wherein ˜ represents a covalent bond to X;

X is CR^{a};

Z is alkyl, cycloalkyl or a monovalent moiety comprising at least one polar functional group when n is 1; or Z is alkylene, cycloalkylene or a polyvalent moiety comprising at least one polar functional group when n is greater than 1;

R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;

R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;

R¹ and R² have, at each occurrence, the following structure, respectively:

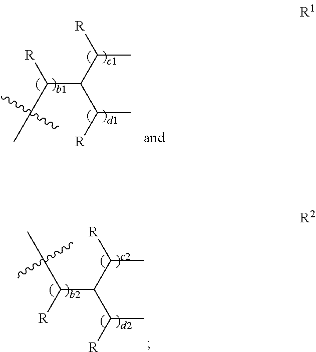

R′ is, at each occurrence, independently H or C₁-C₁₂ alkyl;

a¹ and a² are, at each occurrence, independently an integer from 3 to 12;

b¹ and b² are, at each occurrence, independently 0 or 1;

c¹ and c² are, at each occurrence, independently an integer from 2 to 12;

d¹ and d² are, at each occurrence, independently an integer from 2 to 12;

y is, at each occurrence, independently an integer from 0 to 2; and

n is an integer from 1 to 6,

wherein a¹, a², c¹, c², d¹ and d² are selected such that the sum of a¹+c¹+d¹ is an integer from 18 to 30, and the sum of a²+c²+d² is an integer from 18 to 30, and wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In certain embodiments of Formula (V), G¹ and G² are each independently

### —O(C═O)— or —(C═O)O—.

In other embodiments of Formula (V), X is CH.

In some embodiments of Formula (V), the sum of a¹+c¹+d¹ is an integer from 20 to 30, and the sum of a²+c²+d² is an integer from 18 to 30. In other embodiments, the sum of a¹+c¹+d¹ is an integer from 20 to 30, and the sum of a²+c²+d² is an integer from 20 to 30. In more embodiments of Formula (V), the sum of a¹+b¹+c¹ or the sum of a²+b²+c² is an integer from 12 to 26. In other embodiments, a¹, a², c¹, c², d¹ and d² are selected such that the sum of a¹+c¹+d¹ is an integer from 18 to 28, and the sum of a²+c²+d² is an integer from 18 to 28,

In still other embodiments of Formula (V), a¹ and a² are independently an integer from 3 to 10, for example an integer from 4 to 9.

In yet other embodiments of Formula (V), b¹ and b² are 0. In different embodiments b¹ and b² are 1.

In certain other embodiments of Formula (V), c¹, c², d¹ and d² are independently an integer from 6 to 8.

In different other embodiments of Formula (V), Z is alkyl or a monovalent moiety comprising at least one polar functional group when n is 1; or Z is alkylene or a polyvalent moiety comprising at least one polar functional group when n is greater than 1.

In more embodiments of Formula (V), Z is alkyl, cycloalkyl or a monovalent moiety comprising at least one polar functional group when n is 1. In other embodiments, Z is alkyl.

In other different embodiments of Formula (V), R is, at each occurrence, independently either: (a) H or methyl; or (b) R together with the carbon atom to which it is bound is taken together with an adjacent R and the carbon atom to which it is bound to form a carbon-carbon double bond. For example in some embodiments each R is H. In other embodiments at least one R together with the carbon atom to which it is bound is taken together with an adjacent R and the carbon atom to which it is bound to form a carbon-carbon double bond.

In more embodiments, each R′ is H.

In certain embodiments of Formula (V), the sum of a¹+c¹+d¹ is an integer from 20 to 25, and the sum of a²+c²+d² is an integer from 20 to 25.

In other embodiments of Formula (V), R¹ and R² independently have one of the following structures:

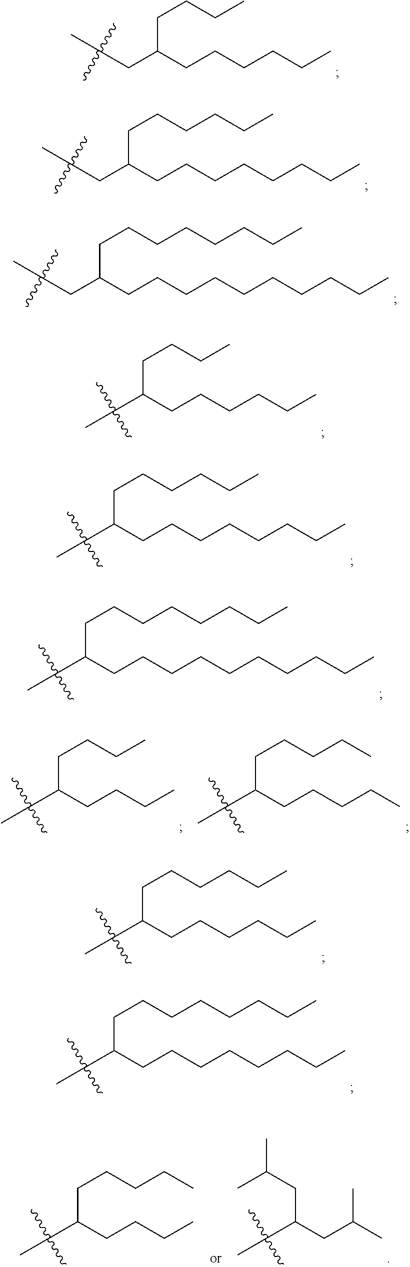

In more embodiments of Formula (V), the compound has one of the following structures:

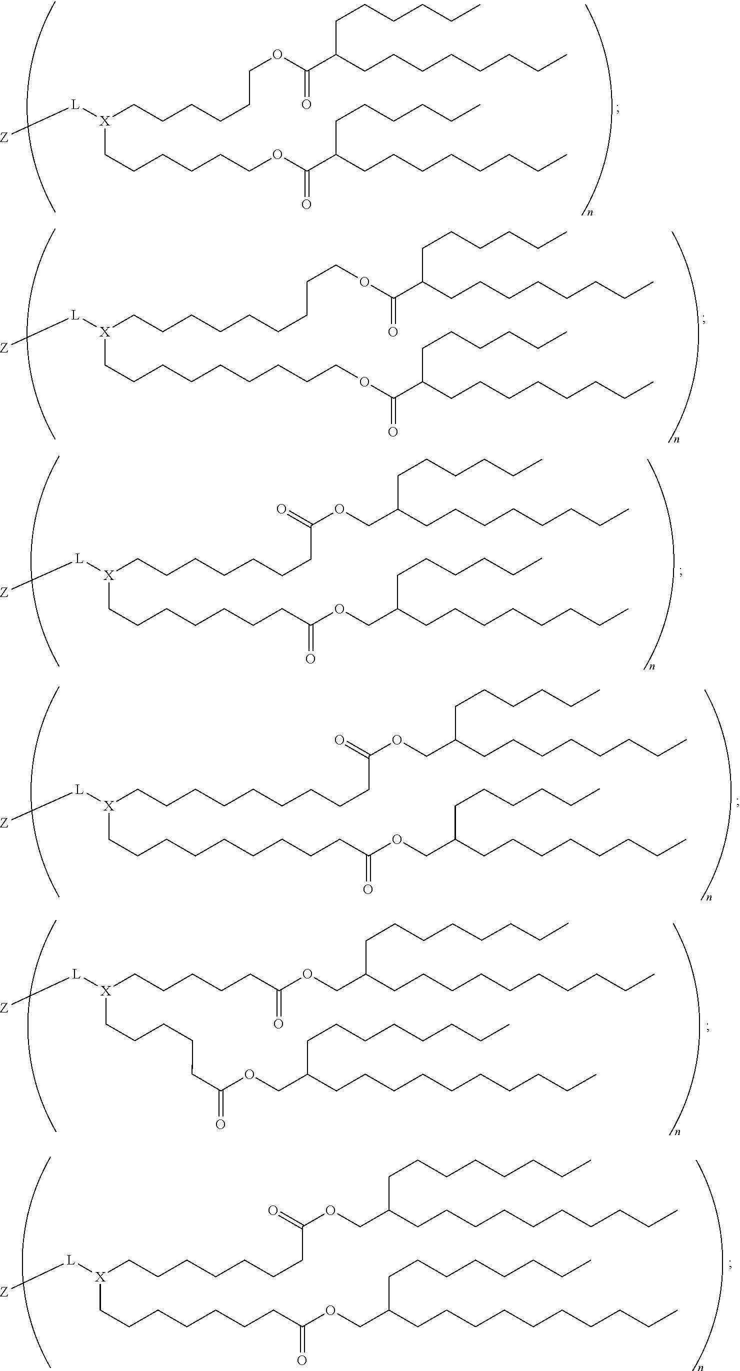

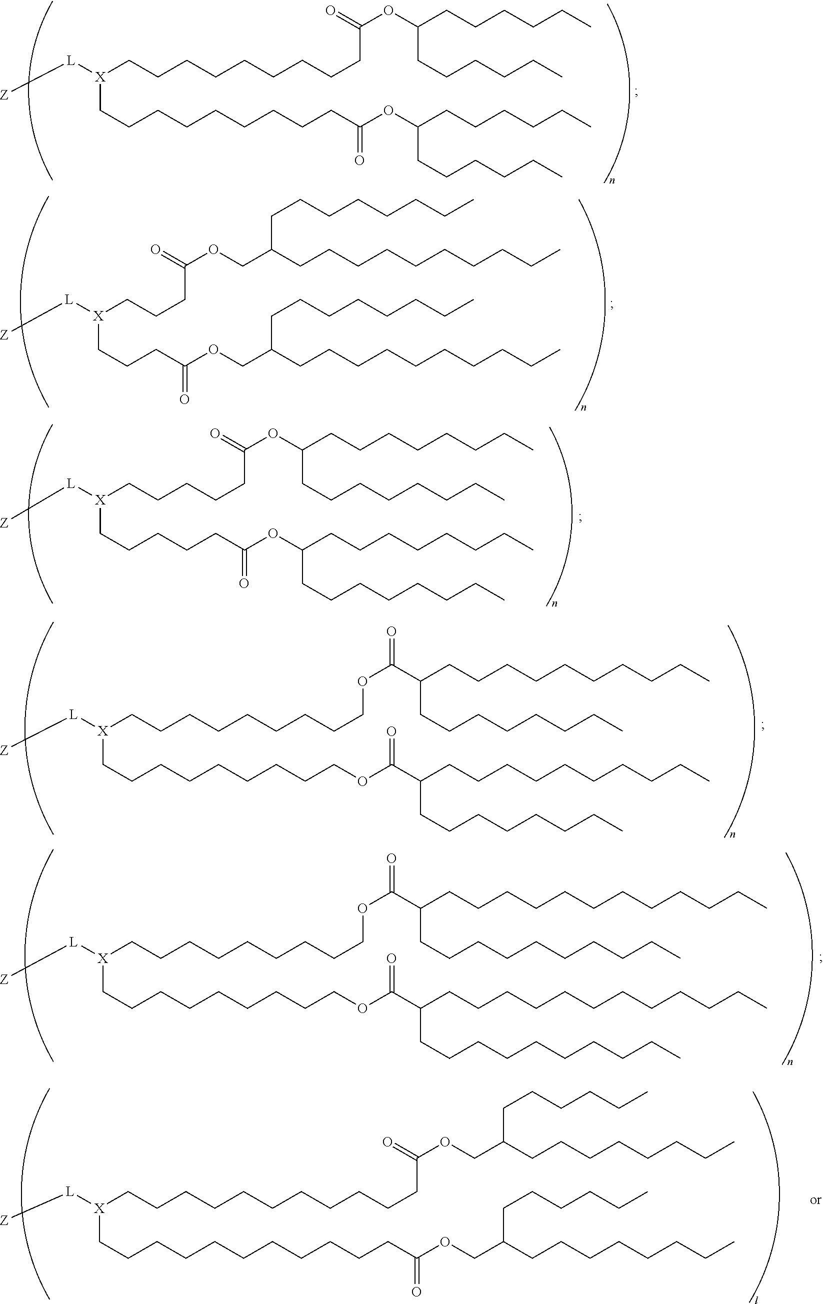

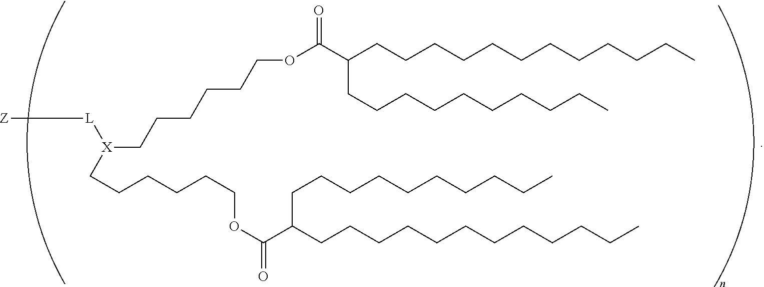

In any of the foregoing embodiments of Formula (IV) or (V), n is 1. In other of the foregoing embodiments of Formula (IV) or (V), n is greater than 1.

In more of any of the foregoing embodiments of Formula (IV) or (V), Z is a mono- or polyvalent moiety comprising at least one polar functional group. In some embodiments, Z is a monovalent moiety comprising at least one polar functional group. In other embodiments, Z is a polyvalent moiety comprising at least one polar functional group.

In more of any of the foregoing embodiments of Formula (IV) or (V), the polar functional group is a hydroxyl, alkoxy, ester, cyano, amide, amino, alkylaminyl, heterocyclyl or heteroaryl functional group.

In any of the foregoing embodiments of Formula (IV) or (V), Z is hydroxyl, hydroxylalkyl, alkoxyalkyl, amino, aminoalkyl, alkylaminyl, alkylaminylalkyl, heterocyclyl or heterocyclylalkyl.

In some other embodiments of Formula (IV) or (V), Z has the following structure:

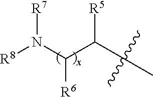

wherein:

R⁵ and R⁶ are independently H or C₁-C₆ alkyl;

R⁷ and R⁸ are independently H or C₁-C₆ alkyl or R⁷ and R⁸, together with the nitrogen atom to which they are attached, join to form a 3-7 membered heterocyclic ring; and

x is an integer from 0 to 6.

In still different embodiments of Formula (IV) or (V), Z has the following structure:

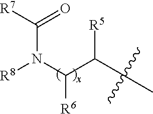

wherein:

R⁵ and R⁶ are independently H or C₁-C₆ alkyl;

R⁷ and R⁸ are independently H or C₁-C₆ alkyl or R⁷ and R⁸, together with the nitrogen atom to which they are attached, join to form a 3-7 membered heterocyclic ring; and

x is an integer from 0 to 6.

In still different embodiments of formula (IV) or (V), Z has the following structure:

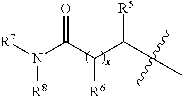

wherein:

R⁵ and R⁶ are independently H or C₁-C₆ alkyl;

R⁷ and R⁸ are independently H or C₁-C₆ alkyl or R⁷ and R⁸, together with the nitrogen atom to which they are attached, join to form a 3-7 membered heterocyclic ring; and

x is an integer from 0 to 6.

In some other embodiments of Formula (IV) or (V), Z is hydroxylalkyl, cyanoalkyl or an alkyl substituted with one or more ester or amide groups.

For example, in any of the foregoing embodiments of Formula (IV) or (V), Z has one of the following structures:

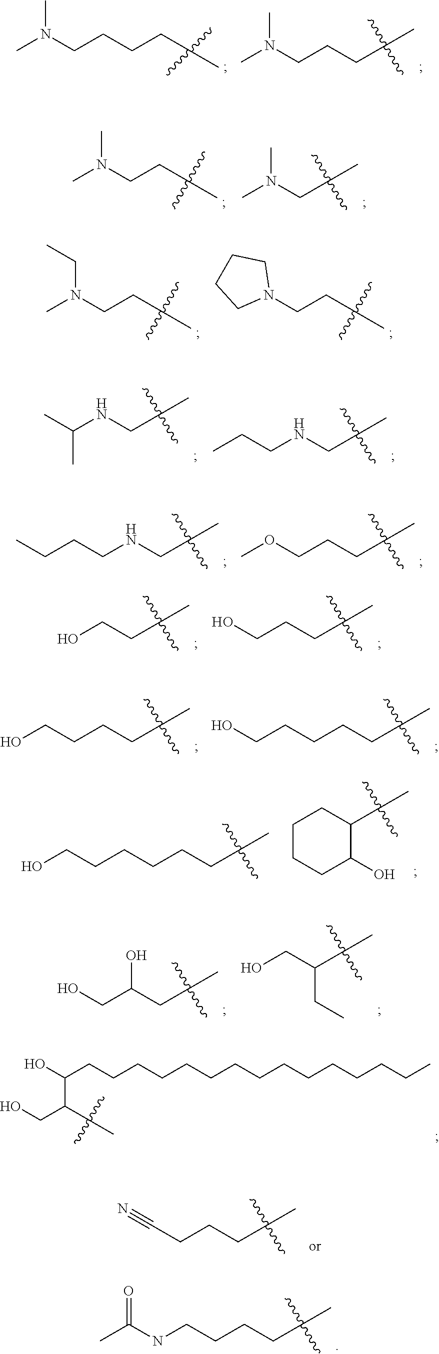

In other embodiments of Formula (IV) or (V), Z-L has one of the following structures:

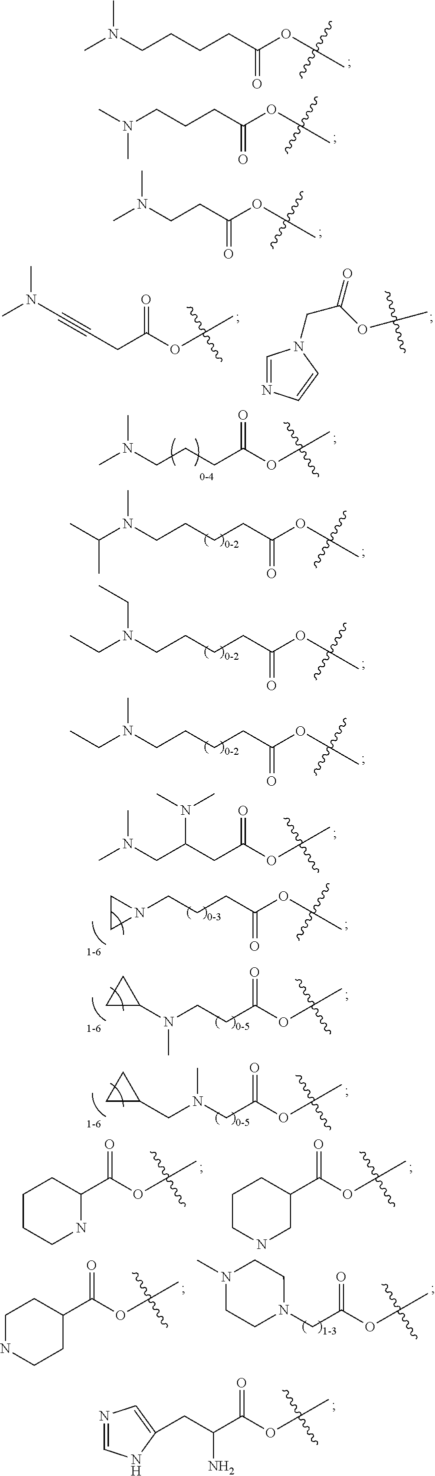

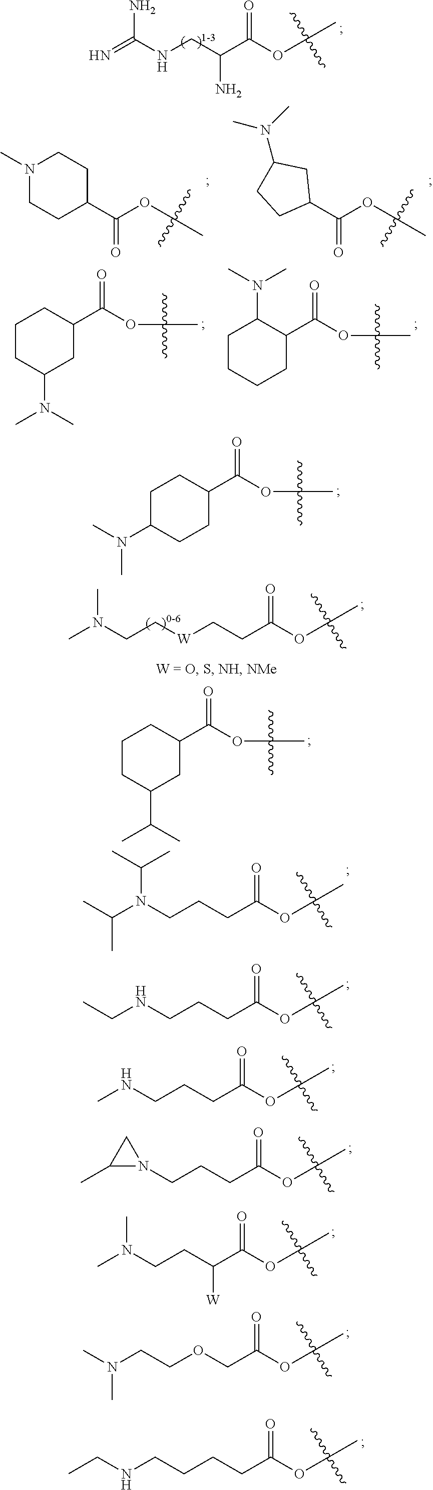

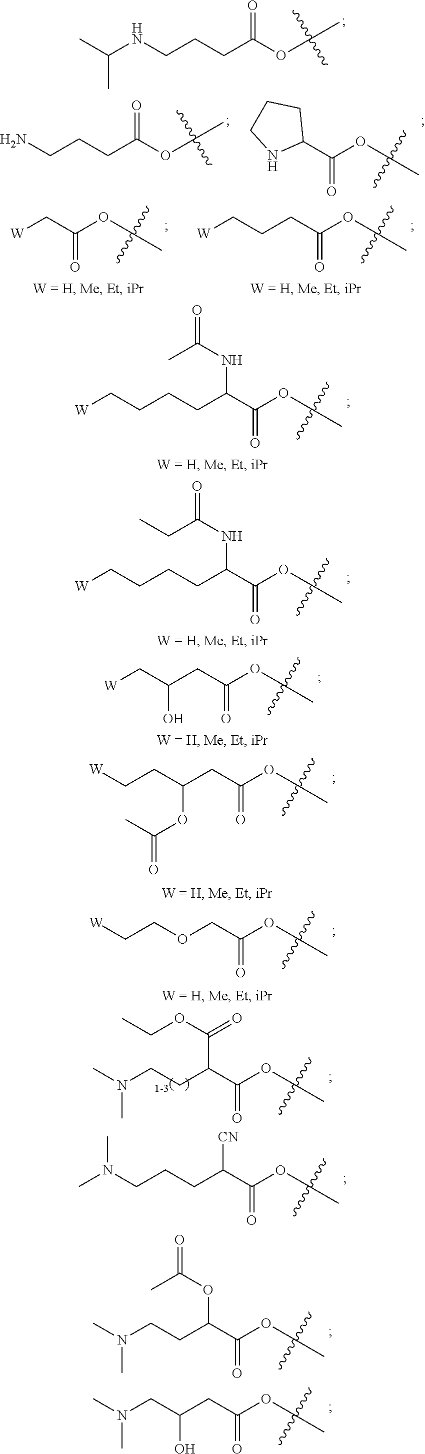

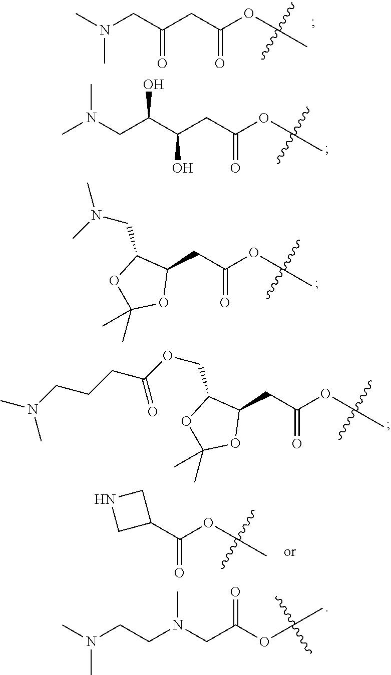

In other embodiments, Z-L has one of the following structures:

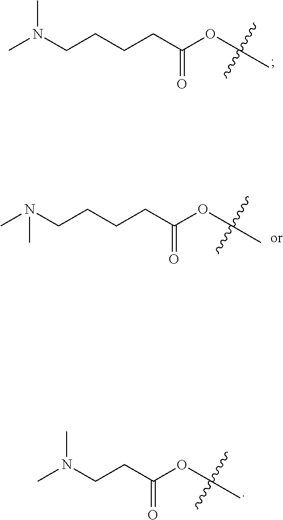

In still other embodiments, X is CH and Z-L has one of the following structures:

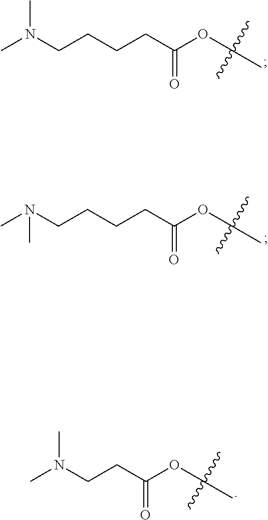

In various different embodiments, a cationic lipid of any one Embodiments 1, 2, 3, 4 or 5 has one of the structures set forth in Table 4 below.

TABLE 4
Representative Compounds of Formula (IV) or (V)

| No. | Structure |
|---|---|
| IV-1 | 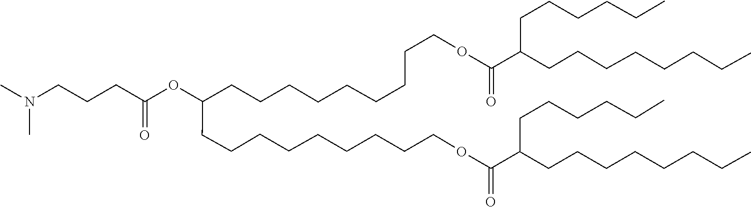 [SMILES: CCCCCCCCC(CCCCCC)C(=O)OCCCCCCCCCC(CCCCCCCCCOC(=O)C(CCCCCC)CCCCCCCC)OC(=O)CCCN(C)C] |
| IV-2 | 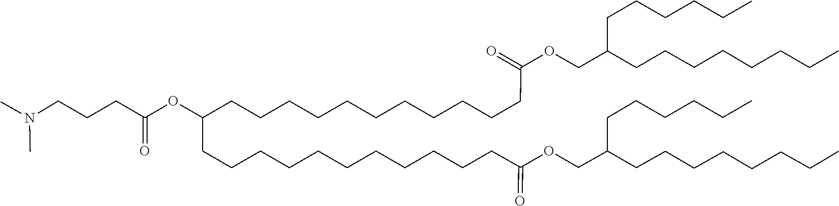 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C] |
| IV-3 | 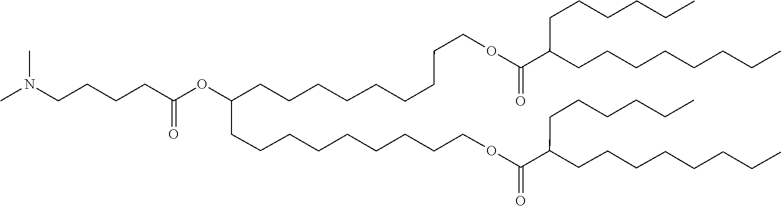 [SMILES: CCCCCCCCC(CCCCCC)C(=O)OCCCCCCCCCC(CCCCCCCCCOC(=O)C(CCCCCC)CCCCCCCC)OC(=O)CCCCN(C)C] |

In one embodiment, the cationic lipid is a compound having the following structure (VI):

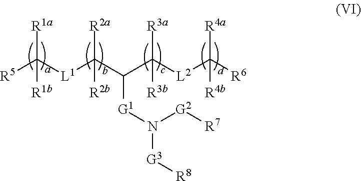

or a pharmaceutically acceptable salt, tautomer, prodrug or stereoisomer thereof, wherein:

L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;

G¹ is C₁-C₂ alkylene, —(C═O)—, —O(C═O)—, —SC(═O)—, —NR^{a}C(═O)— or a direct bond;

G² is —C(═O)—, —(C═O)O—, —C(═O)S—, —C(═O)NR^{a}— or a direct bond;

G³ is C₁-C₆ alkylene;

R^{a} is H or C₁-C₁₂ alkyl;

R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;

R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;

R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;

R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;

R⁵ and R⁶ are each independently H or methyl;

R⁷ is H or C₁-C₂₀ alkyl;

R⁸ is OH, —N(R⁹)(C═O)R¹⁰, —(C═O)NR⁹R¹⁰, —NR⁹R¹⁰, —(C═O)OR¹¹ or —O(C═O)R¹¹, provided that G³ is C₄-C₆ alkylene when R⁸ is —NR⁹R¹⁰,

R⁹ and R¹⁰ are each independently H or C₁-C₁₂ alkyl;

R¹¹ is aralkyl;

a, b, c and d are each independently an integer from 1 to 24; and

x is 0, 1 or 2,

wherein each alkyl, alkylene and aralkyl is optionally substituted.

In some embodiments of structure (VI), L¹ and L² are each independently —O(C═O)—, —(C═O)O— or a direct bond. In other embodiments, G¹ and G² are each independently —(C═O)— or a direct bond. In some different embodiments, L¹ and L² are each independently —O(C═O)—, —(C═O)O— or a direct bond; and G¹ and G² are each independently —(C═O)— or a direct bond.

In some different embodiments of structure (VI), L¹ and L² are each independently —C(═O)—, —O—, —S(O)ₓ—, —S—S—, —C(═O)S—, —SC(═O)—, —NR^{a}—, —NR^{a}C(═O)—, —C(═O)NR^{a}—, —NR^{a}C(═O)NR^{a}, —OC(═O)NR^{a}—, —NR^{a}C(═O)O—, S(O)ₓNR^{a}—, —NR^{a}S(O)ₓ— or —S(O)ₓNR^{a}—.

In other of the foregoing embodiments of structure (VI), the compound has one of the following structures (VIA) or (VIB):

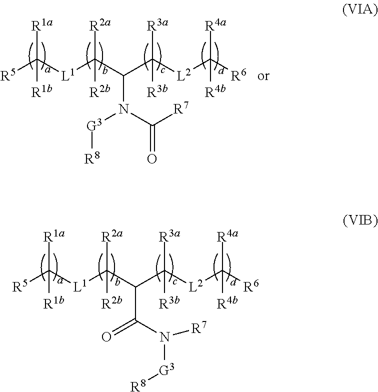

In some embodiments, the compound has structure (VIA). In other embodiments, the compound has structure (VIB).

In any of the foregoing embodiments of structure (VI), one of L¹ or L² is —O(C═O)—. For example, in some embodiments each of L¹ and L² are —O(C═O)—.

In some different embodiments of any of the foregoing, one of L¹ or L² is —(C═O)O—. For example, in some embodiments each of L¹ and L² is —(C═O)O—.

In different embodiments of structure (VI), one of L¹ or L² is a direct bond. As used herein, a “direct bond” means the group (e.g., L¹ or L²) is absent. For example, in some embodiments each of L¹ and L² is a direct bond.

In other different embodiments of the foregoing, for at least one occurrence of R^{1d} and R^{1b}, R^{1a} is H or C₁-C₁₂ alkyl, and R^{b} together with the carbon atom to which it is bound is taken together with an adjacent R^{1b} and the carbon atom to which it is bound to form a carbon-carbon double bond.

In still other different embodiments of structure (VI), for at least one occurrence of R^{4a} and R^{4b}, R^{4a} is H or C₁-C₁₂ alkyl, and R^{4b} together with the carbon atom to which it is bound is taken together with an adjacent R^{4b} and the carbon atom to which it is bound to form a carbon-carbon double bond.

In more embodiments of structure (VI), for at least one occurrence of R^{2a} and R^{2b}, R^{2a} is H or C₁-C₁₂ alkyl, and R^{2b} together with the carbon atom to which it is bound is taken together with an adjacent R^{2b} and the carbon atom to which it is bound to form a carbon-carbon double bond.

In other different embodiments of any of the foregoing, for at least one occurrence of R^{3a} and R^{3b}, R^{3a} is H or C₁-C₁₂ alkyl, and R^{3b} together with the carbon atom to which it is bound is taken together with an adjacent R^{3b} and the carbon atom to which it is bound to form a carbon-carbon double bond.

It is understood that “carbon-carbon” double bond refers to one of the following structures:

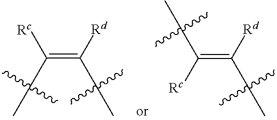

wherein R^{c} and R^{d} are, at each occurrence, independently H or a substituent. For example, in some embodiments R^{c} and R^{d} are, at each occurrence, independently H, C₁-C₁₂ alkyl or cycloalkyl, for example H or C₁-C₁₂ alkyl.

In various other embodiments, the compound has one of the following structures (VIC) or (VID):

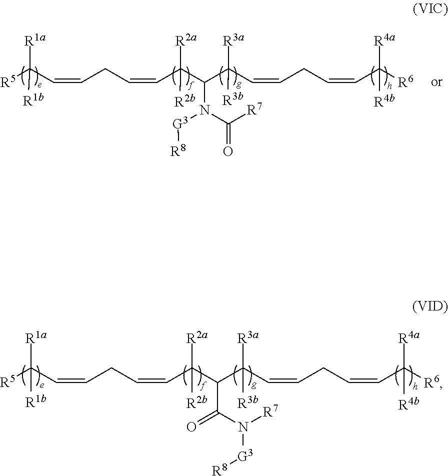

wherein e, f, g and h are each independently an integer from 1 to 12.

In some embodiments, the compound has structure (VIC). In other embodiments, the compound has structure (VID).

In various embodiments of the compounds of structures (VIC) or (VID), e, f, g and h are each independently an integer from 4 to 10.

In other different embodiments,

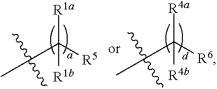

or both, independently has one of the following structures:

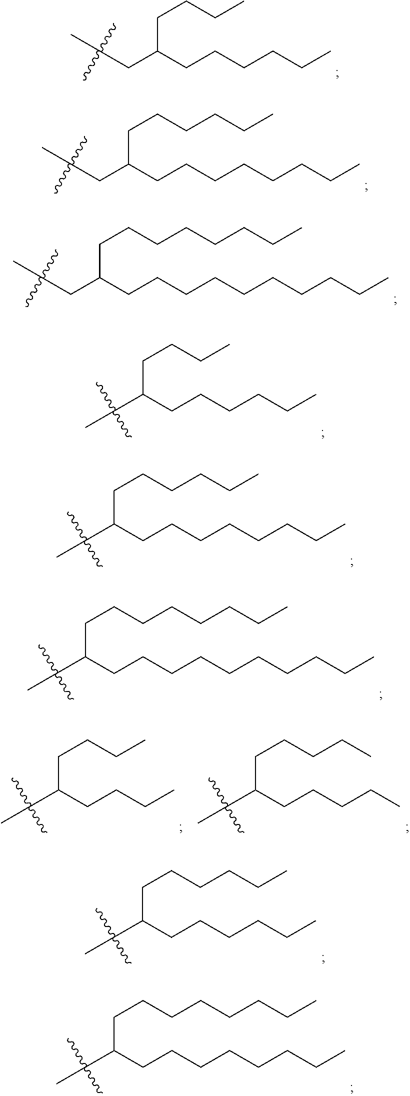

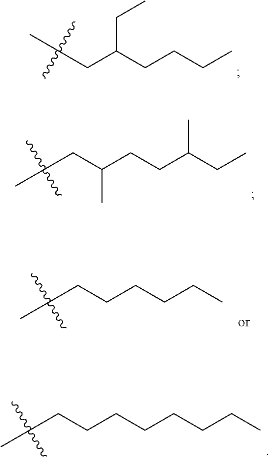

In certain embodiments of the foregoing, a, b, c and d are each independently an integer from 2 to 12 or an integer from 4 to 12. In other embodiments, a, b, c and d are each independently an integer from 8 to 12 or 5 to 9. In some certain embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some embodiments of structure (VI), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some embodiments of structure (VI), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some certain embodiments of structure (VI), d is 0. In some embodiments, d is 1. In other embodiments, d is 2. In more embodiments, d is 3. In yet other embodiments, d is 4. In some embodiments, d is 5. In other embodiments, d is 6. In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some embodiments of structure (VI), e is 1. In other embodiments, e is 2. In more embodiments, e is 3. In yet other embodiments, e is 4. In some embodiments, e is 5. In other embodiments, e is 6. In more embodiments, e is 7. In yet other embodiments, e is 8. In some embodiments, e is 9. In other embodiments, e is 10. In more embodiments, e is 11. In yet other embodiments, e is 12.

In some embodiments of structure (VI), f is 1. In other embodiments, f is 2. In more embodiments, f is 3. In yet other embodiments, f is 4. In some embodiments, f is 5. In other embodiments, f is 6. In more embodiments, f is 7. In yet other embodiments, f is 8. In some embodiments, f is 9. In other embodiments, f is 10. In more embodiments, f is 11. In yet other embodiments, f is 12.

In some embodiments of structure (VI), g is 1. In other embodiments, g is 2. In more embodiments, g is 3. In yet other embodiments, g is 4. In some embodiments, g is 5. In other embodiments, g is 6. In more embodiments, g is 7. In yet other embodiments, g is 8. In some embodiments, g is 9. In other embodiments, g is 10. In more embodiments, g is 11. In yet other embodiments, g is 12.

In some embodiments of structure (VI), h is 1. In other embodiments, e is 2. In more embodiments, h is 3. In yet other embodiments, h is 4. In some embodiments, e is 5. In other embodiments, h is 6. In more embodiments, h is 7. In yet other embodiments, h is 8. In some embodiments, h is 9. In other embodiments, h is 10. In more embodiments, h is 11. In yet other embodiments, his 12.

In some other various embodiments of structure (VI), a and d are the same. In some other embodiments, b and c are the same. In some other specific embodiments a and d are the same and b and c are the same.

The sum of a and b and the sum of c and d are factors which may be varied to obtain a lipid having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a. b, c and d are selected such that the sum of a and b and the sum of c and d is 12 or greater.

The substituents at R^{1a}, R^{2a}, R^{3a} and R^{4a} are not particularly limited. In some embodiments, at least one of R^{1a}, R^{2a}, R^{3a} and R^{4a} is H. In certain embodiments R^{1a}, R^{2a}, R^{3a} and R^{4a} are H at each occurrence. In certain other embodiments at least one of R^{1a}, R^{2a}, R^{3a} and R^{4a} is C₁-C₁₂ alkyl. In certain other embodiments at least one of R^{1a}, R^{2a}, R^{3a} and R^{4a} is C₁₋C₈ alkyl. In certain other embodiments at least one of R^{1a}, R^{2a}, R^{3a} and R^{4a} is C₁-C₆ alkyl. In some of the foregoing embodiments, the C₁-C₈ alkyl is methyl, ethyl, n-propyl, iso-propyl, n-butyl, iso-butyl, tert-butyl, n-hexyl or n-octyl.

In certain embodiments of the foregoing, R^{1a}, R^{1b}, R^{4a} and R^{4b} are C₁-C₁₂ alkyl at each occurrence.

In further embodiments of the foregoing, at least one of R^{1b}, R^{2b}, R^{3b} and R^{4b} is H or Rib, R^{2b}, R^{3b} and R^{4b} are H at each occurrence.

In certain embodiments of the foregoing, R^{1b} together with the carbon atom to which it is bound is taken together with an adjacent R^{1b} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R⁵ and R⁶ are not particularly limited in the foregoing embodiments. In certain embodiments one of R⁵ or R⁶ is methyl. In other embodiments each of R⁵ or R⁶ is methyl.

The substituents at R⁷ are not particularly limited in the foregoing embodiments. In certain embodiments R⁷ is C₆-C₁₆ alkyl. In some other embodiments, R⁷ is C₆-C₉ alkyl. In some of these embodiments, R⁷ is substituted with —(C═O)OR^{b}, —O(C═O)R^{b}, —C(═O)R^{b}, —OR^{b}, —S(O)ₓR^{b}, —S—SR^{b}, —C(═O)SR^{b}, —SC(═O)R^{b}, —NR^{a}R^{b}, —NR^{a}C(═O)R^{b}, —C(═O)NR^{a}R^{b}, —NR^{a}C(═O)NR^{a}R^{b}, —OC(═O)NR^{a}R^{b}, —NR^{a}C(═O)OR^{b}, —NR^{a}S(O)ₓNR^{a}R^{b}, —NR^{a}S(O)ₓR^{b} or —S(O)ₓNR^{a}R^{b}, wherein: R^{a} is H or C₁-C₁₂ alkyl; R^{b} is C₁-C₁₅ alkyl; and x is 0, 1 or 2. For example, in some embodiments R⁷ is substituted with —(C═O)OR^{b} or —O(C═O)R^{b}.

In various of the foregoing embodiments of structure (VI), R^{b} is branched C₃-C₁₅ alkyl. For example, in some embodiments R^{b} has one of the following structures:

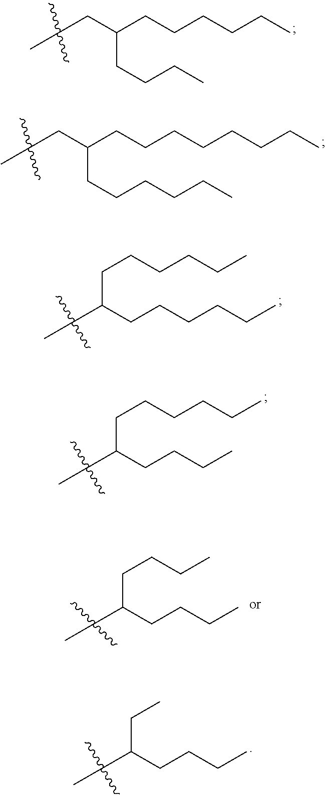

In certain embodiments, R⁸ is OH.

In other embodiments of structure (VI), R⁸ is —N(R⁹)(C═O)R¹⁰. In some other embodiments, R⁸ is —(C═O)NR⁹R¹⁰. In still more embodiments, R⁸ is —N(R⁹)(C═O)R¹⁰. In some of the foregoing embodiments, R⁹ and R¹⁰ are each independently H or C₁-C₈ alkyl, for example H or C₁-C₃ alkyl. In more specific of these embodiments, the C₁-C₈ alkyl or C₁-C₃ alkyl is unsubstituted or substituted with hydroxyl. In other of these embodiments, R⁹ and R¹⁰ are each methyl.

In yet more embodiments of structure (VI), R⁸ is —(C═O)OR¹¹. In some of these embodiments R¹¹ is benzyl.

In yet more specific embodiments of structure (VI), R⁸ has one of the following structures:

—OH;

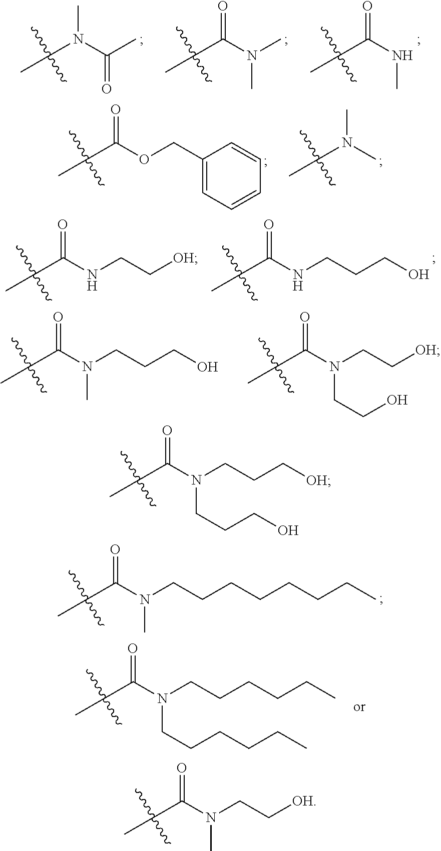

In still other embodiments of the foregoing compounds, G³ is C₂-C₅ alkylene, for example C₂-C₄ alkylene, C₃ alkylene or C₄ alkylene. In some of these embodiments, R⁸ is OH. In other embodiments, G² is absent and R⁷ is C₁-C₂ alkylene, such as methyl.

In various different embodiments, the compound has one of the structures set forth in Table 5 below.

TABLE 5
Representative cationic lipids of structure (VI)

| No. | Structure |
|---|---|
| VI-1 | 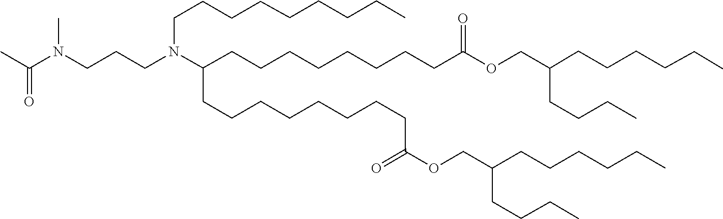 [SMILES: CCCCCCCCCN(CCCN(C)C(C)=O)C(CCCCCCCCC(=O)OCC(CCCC)CCCCCC)CCCCCCCCC(=O)OCC(CCCC)CCCCCC] |
| VI-2 | 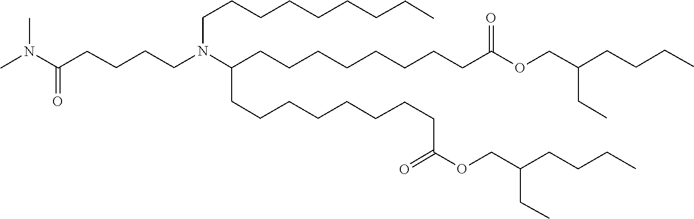 [SMILES: CCCCCCCCCN(CCCCC(=O)N(C)C)C(CCCCCCCCC(=O)OCC(CC)CCCC)CCCCCCCCC(=O)OCC(CC)CCCC] |
| VI-3 | 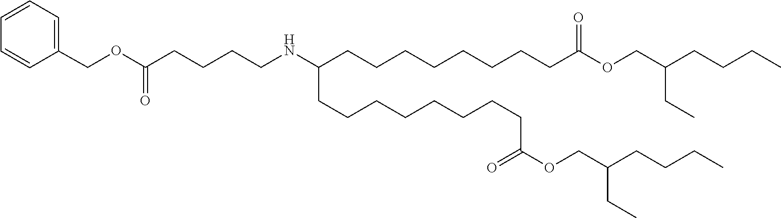 [SMILES: CCCCC(CC)COC(=O)CCCCCCCCC(CCCCCCCCC(=O)OCC(CC)CCCC)NCCCCC(=O)OCc1ccccc1] |
| VI-4 | 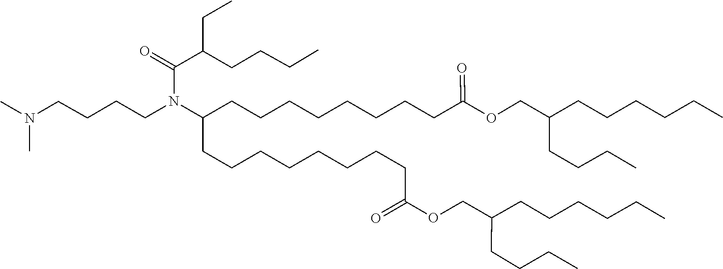 [SMILES: CCCCCCC(CCCC)COC(=O)CCCCCCCCC(CCCCCCCCC(=O)OCC(CCCC)CCCCCC)N(CCCCN(C)C)C(=O)C(CC)CCCC] |
| VI-5 | 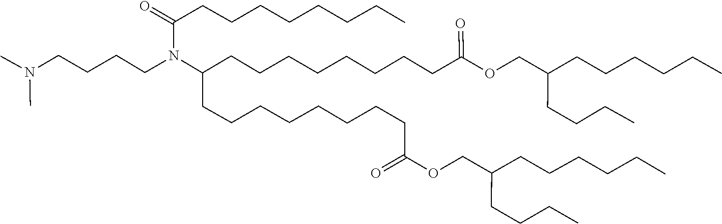 [SMILES: CCCCCCCCC(=O)N(CCCCN(C)C)C(CCCCCCCCC(=O)OCC(CCCC)CCCCCC)CCCCCCCCC(=O)OCC(CCCC)CCCCCC] |
| VI-6 | 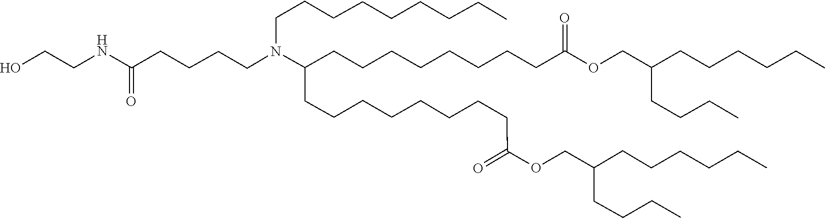 [SMILES: CCCCCCCCCN(CCCCC(=O)NCCO)C(CCCCCCCCC(=O)OCC(CCCC)CCCCCC)CCCCCCCCC(=O)OCC(CCCC)CCCCCC] |
| VI-7 | 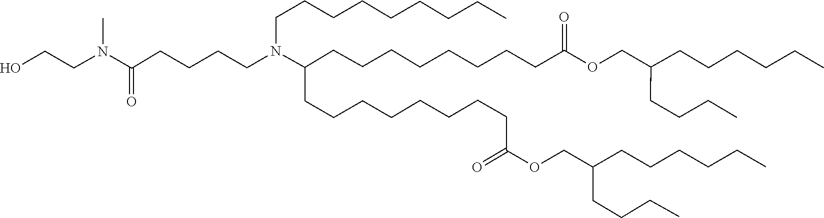 [SMILES: CCCCCCCCCN(CCCCC(=O)N(C)CCO)C(CCCCCCCCC(=O)OCC(CCCC)CCCCCC)CCCCCCCCC(=O)OCC(CCCC)CCCCCC] |
| VI-8 | 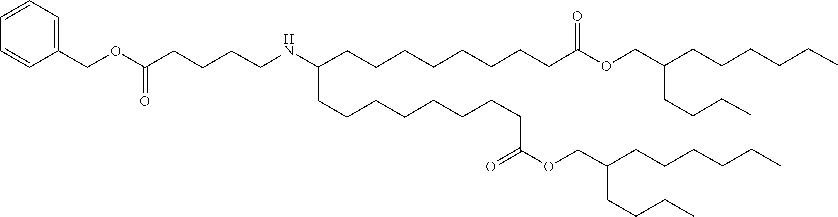 [SMILES: CCCCCCC(CCCC)COC(=O)CCCCCCCCC(CCCCCCCCC(=O)OCC(CCCC)CCCCCC)NCCCCC(=O)OCc1ccccc1] |
| VI-9 | 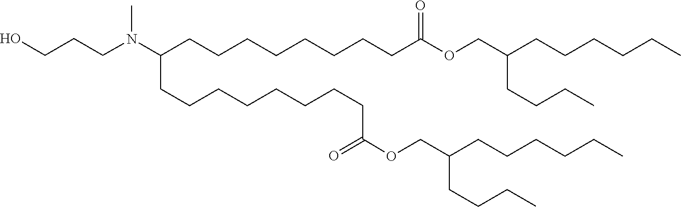 [SMILES: CCCCCCC(CCCC)COC(=O)CCCCCCCCC(CCCCCCCCC(=O)OCC(CCCC)CCCCCC)N(C)CCCO] |
| VI-10 | 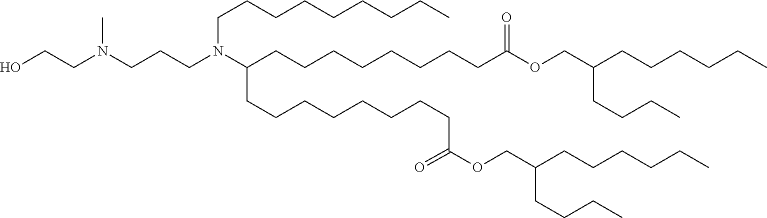 [SMILES: CCCCCCCCCN(CCCN(C)CCO)C(CCCCCCCCC(=O)OCC(CCCC)CCCCCC)CCCCCCCCC(=O)OCC(CCCC)CCCCCC] |
| VI-11 | 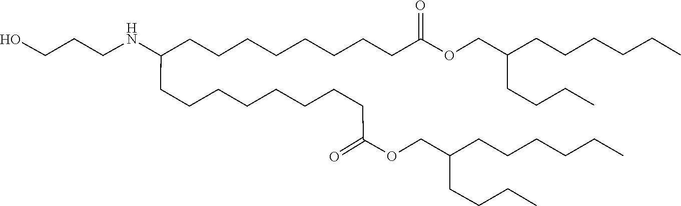 [SMILES: CCCCCCC(CCCC)COC(=O)CCCCCCCCC(CCCCCCCCC(=O)OCC(CCCC)CCCCCC)NCCCO] |
| VI-12 | 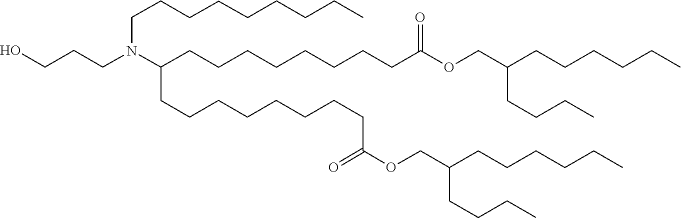 [SMILES: CCCCCCCCCN(CCCO)C(CCCCCCCCC(=O)OCC(CCCC)CCCCCC)CCCCCCCCC(=O)OCC(CCCC)CCCCCC] |
| VI-13 | 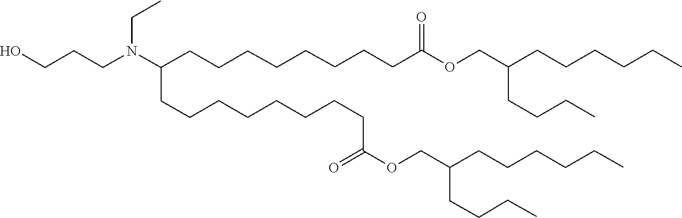 [SMILES: CCCCCCC(CCCC)COC(=O)CCCCCCCCC(CCCCCCCCC(=O)OCC(CCCC)CCCCCC)N(CC)CCCO] |
| VI-14 | 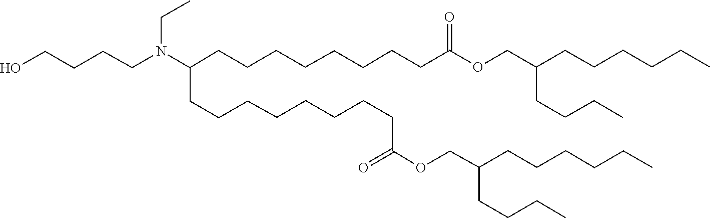 [SMILES: CCCCCCC(CCCC)COC(=O)CCCCCCCCC(CCCCCCCCC(=O)OCC(CCCC)CCCCCC)N(CC)CCCCO] |
| VI-15 | 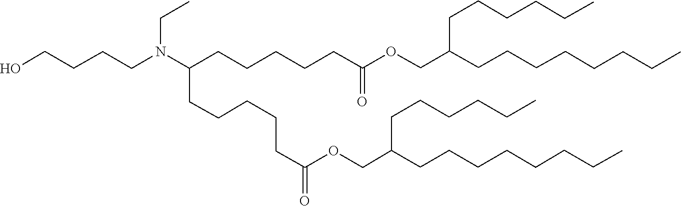 [SMILES: CCCCCCCCC(CCCCCC)COC(=O)CCCCCC(CCCCCC(=O)OCC(CCCCCC)CCCCCCCC)N(CC)CCCCO] |
| VI-16 | 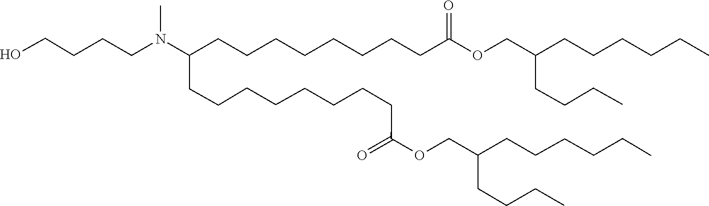 [SMILES: CCCCCCC(CCCC)COC(=O)CCCCCCCCC(CCCCCCCCC(=O)OCC(CCCC)CCCCCC)N(C)CCCCO] |
| VI-17 | 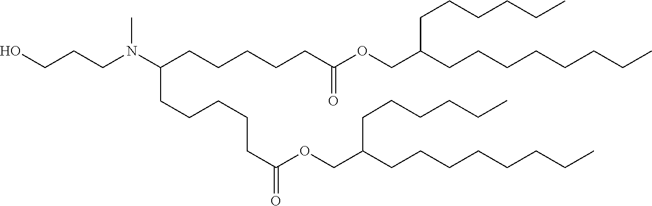 [SMILES: CCCCCCCCC(CCCCCC)COC(=O)CCCCCC(CCCCCC(=O)OCC(CCCCCC)CCCCCCCC)N(C)CCCO] |
| VI-18 | 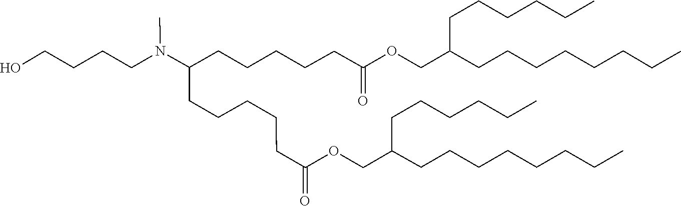 [SMILES: CCCCCCCCC(CCCCCC)COC(=O)CCCCCC(CCCCCC(=O)OCC(CCCCCC)CCCCCCCC)N(C)CCCCO] |
| VI-19 | 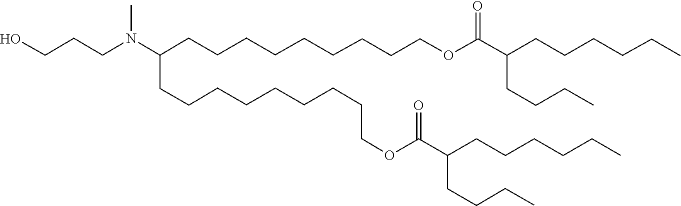 [SMILES: CCCCCCC(CCCC)C(=O)OCCCCCCCCCC(CCCCCCCCCOC(=O)C(CCCC)CCCCCC)N(C)CCCO] |
| VI-20 | 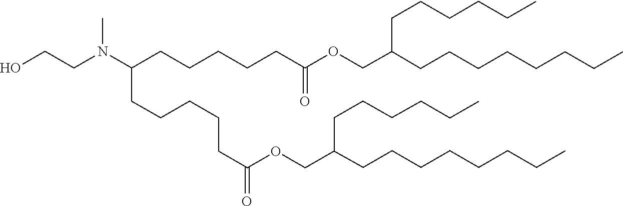 [SMILES: CCCCCCCCC(CCCCCC)COC(=O)CCCCCC(CCCCCC(=O)OCC(CCCCCC)CCCCCCCC)N(C)CCO] |
| VI-21 | 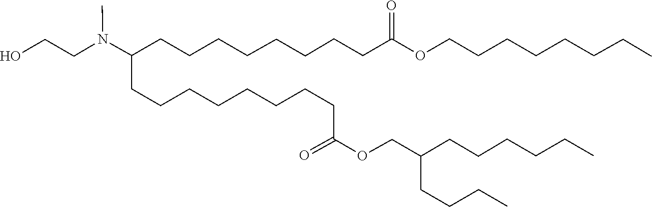 [SMILES: CCCCCCCCOC(=O)CCCCCCCCC(CCCCCCCCC(=O)OCC(CCCC)CCCCCC)N(C)CCO] |
| VI-22 | 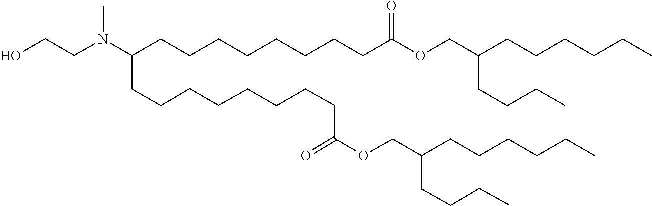 [SMILES: CCCCCCC(CCCC)COC(=O)CCCCCCCCC(CCCCCCCCC(=O)OCC(CCCC)CCCCCC)N(C)CCO] |
| VI-23 | 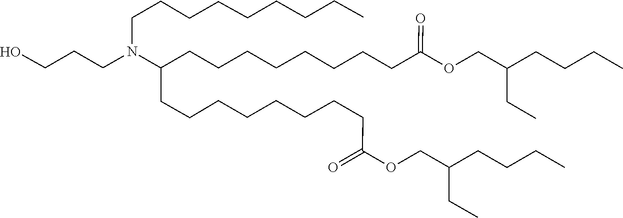 [SMILES: CCCCCCCCCN(CCCO)C(CCCCCCCCC(=O)OCC(CC)CCCC)CCCCCCCCC(=O)OCC(CC)CCCC] |
| VI-24 | 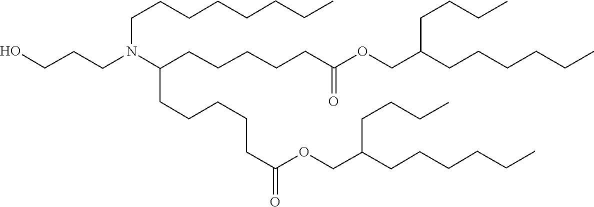 [SMILES: CCCCCCCCN(CCCO)C(CCCCCC(=O)OCC(CCCC)CCCCCC)CCCCCC(=O)OCC(CCCC)CCCCCC] |
| VI-25 | 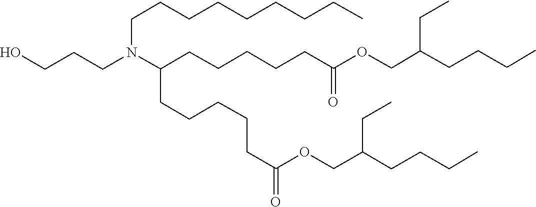 [SMILES: CCCCCCCCCN(CCCO)C(CCCCCC(=O)OCC(CC)CCCC)CCCCCC(=O)OCC(CC)CCCC] |
| VI-26 | 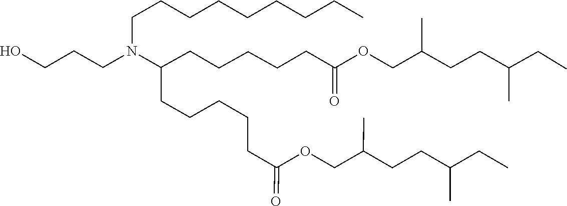 [SMILES: CCCCCCCCCN(CCCO)C(CCCCCC(=O)OCC(C)CCC(C)CC)CCCCCC(=O)OCC(C)CCC(C)CC] |
| VI-27 | 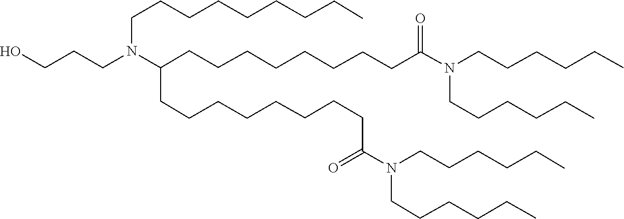 [SMILES: CCCCCCCCCN(CCCO)C(CCCCCCCCC(=O)N(CCCCCC)CCCCCC)CCCCCCCCC(=O)N(CCCCCC)CCCCCC] |
| VI-28 | 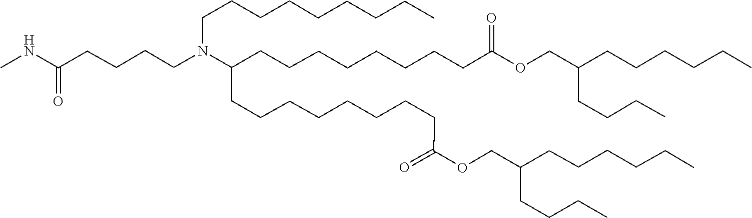 [SMILES: CCCCCCCCCN(CCCCC(=O)NC)C(CCCCCCCCC(=O)OCC(CCCC)CCCCCC)CCCCCCCCC(=O)OCC(CCCC)CCCCCC] |
| VI-29 | 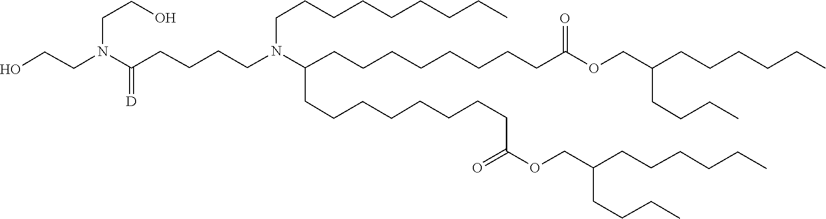 |
| VI-30 | 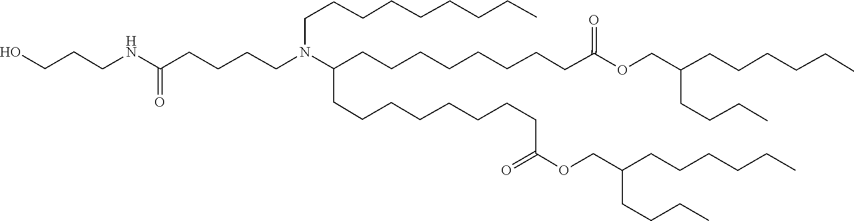 [SMILES: CCCCCCCCCN(CCCCC(=O)NCCCO)C(CCCCCCCCC(=O)OCC(CCCC)CCCCCC)CCCCCCCCC(=O)OCC(CCCC)CCCCCC] |
| VI-31 | 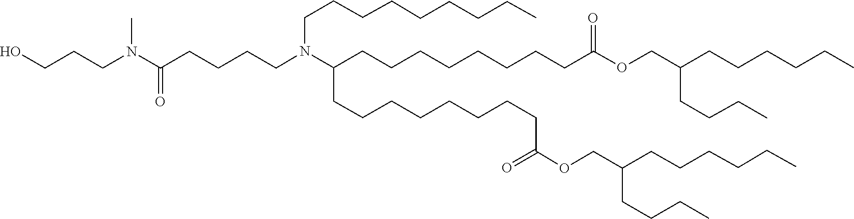 [SMILES: CCCCCCCCCN(CCCCC(=O)N(C)CCCO)C(CCCCCCCCC(=O)OCC(CCCC)CCCCCC)CCCCCCCCC(=O)OCC(CCCC)CCCCCC] |
| VI-32 | 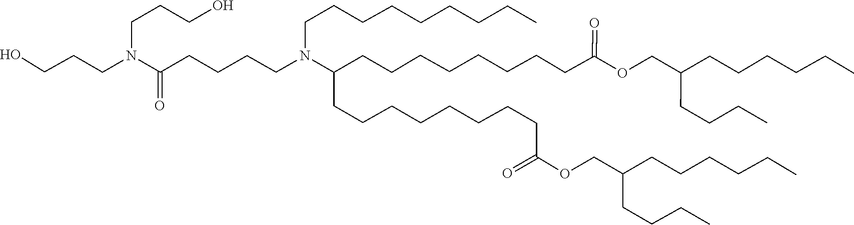 [SMILES: CCCCCCCCCN(CCCCC(=O)N(CCCO)CCCO)C(CCCCCCCCC(=O)OCC(CCCC)CCCCCC)CCCCCCCCC(=O)OCC(CCCC)CCCCCC] |
| VI-33 | 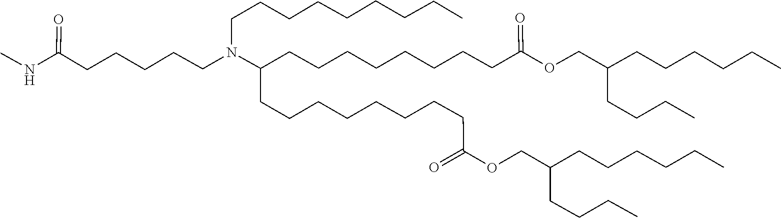 [SMILES: CCCCCCCCCN(CCCCCC(=O)NC)C(CCCCCCCCC(=O)OCC(CCCC)CCCCCC)CCCCCCCCC(=O)OCC(CCCC)CCCCCC] |
| VI-34 | 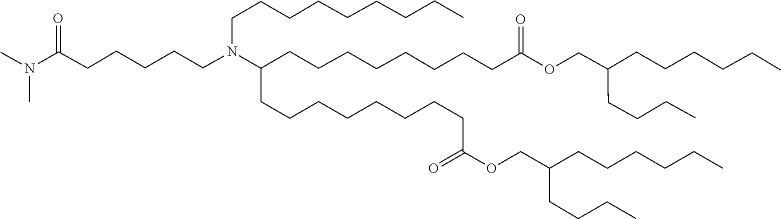 [SMILES: CCCCCCCCCN(CCCCCC(=O)N(C)C)C(CCCCCCCCC(=O)OCC(CCCC)CCCCCC)CCCCCCCCC(=O)OCC(CCCC)CCCCCC] |
| VI-35 | 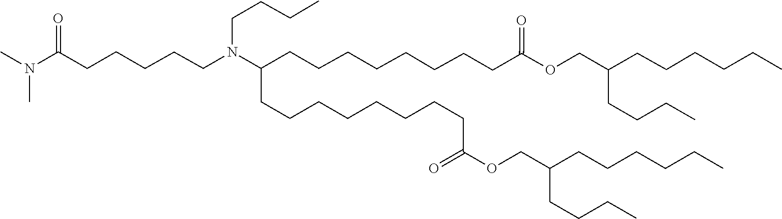 [SMILES: CCCCCCC(CCCC)COC(=O)CCCCCCCCC(CCCCCCCCC(=O)OCC(CCCC)CCCCCC)N(CCCC)CCCCCC(=O)N(C)C] |
| VI-36 | 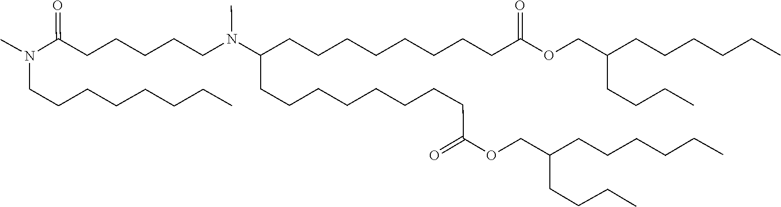 [SMILES: CCCCCCCCN(C)C(=O)CCCCCN(C)C(CCCCCCCCC(=O)OCC(CCCC)CCCCCC)CCCCCCCCC(=O)OCC(CCCC)CCCCCC] |
| VI-37 | 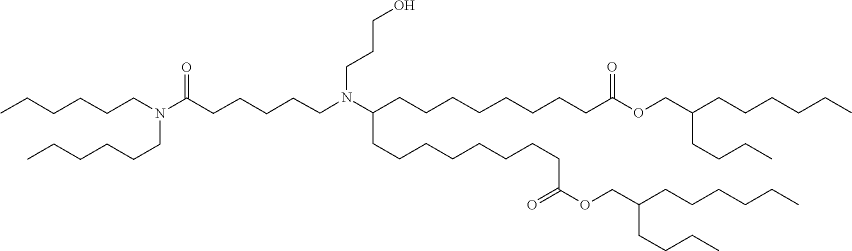 [SMILES: CCCCCCC(CCCC)COC(=O)CCCCCCCCC(CCCCCCCCC(=O)OCC(CCCC)CCCCCC)N(CCCO)CCCCCC(=O)N(CCCCCC)CCCCCC] |

In one embodiment, the cationic lipid is a compound having the following structure (VII):

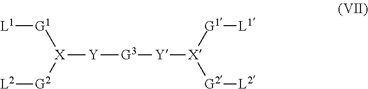

or a pharmaceutically acceptable salt, prodrug or stereoisomer thereof, wherein:

X and X′ are each independently N or CR;

Y and Y′ are each independently absent, —O(C═O)—, —(C═O)O— or NR, provided that:
a)Y is absent when X is N;
b) Y′ is absent when X′ is N;
c) Y is —O(C═O)—, —(C═O)O— or NR when X is CR; and
d) Y′ is —O(C═O)—, —(C═O)O— or NR when X is CR,

L¹ and L^{1′} are each independently —O(C═O)R¹, —(C═O)OR¹, —C(═O)R¹, —OR¹, —S(O)_{z}R¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;

L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;

G¹, G^{1′}, G² and G^{2′} are each independently C₂-C₁₂ alkylene or C₂-C₁₂ alkenylene;

G³ is C₂-C₂₄ heteroalkylene or C₂-C₂₄ heteroalkenylene;

R^{a}, R^{b}, R^{d} and R^{e} are, at each occurrence, independently H, C₁-C₁₂ alkyl or C₂-C₁₂ alkenyl;

R^{e} and R^{f} are, at each occurrence, independently C₁-C₁₂ alkyl or C₂-C₁₂ alkenyl;

R is, at each occurrence, independently H or C₁-C₁₂ alkyl;

R¹ and R² are, at each occurrence, independently branched C₆-C₂₄ alkyl or branched C₆-C₂₄ alkenyl;

z is 0, 1 or 2, and

wherein each alkyl, alkenyl, alkylene, alkenylene, heteroalkylene and heteroalkenylene is independently substituted or unsubstituted unless otherwise specified.

In other different embodiments of structure (VII):

X and X′ are each independently N or CR;

Y and Y′ are each independently absent or NR, provided that:
a)Y is absent when X is N;
b) Y′ is absent when X′ is N;
c) Y is NR when X is CR, and
d) Y′ is NR when X′ is CR,

L¹ and L^{1′} are each independently —O(C═O)R¹, —(C═O)OR¹, —C(═O)R¹, —OR¹, —S(O)_{z}R¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;

L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;

G¹, G^{1′}, G² and G^{2′} are each independently C₂-C₁₂ alkylene or C₂-C₁₂ alkenylene;

G³ is C₂-C₂₄ alkyleneoxide or C₂-C₂₄ alkenyleneoxide;

R^{a}, R^{b}, R^{d} and R^{e} are, at each occurrence, independently H, C₁-C₁₂ alkyl or C₂-C₁₂ alkenyl;

R^{e} and R^{f} are, at each occurrence, independently C₁-C₁₂ alkyl or C₂-C₁₂ alkenyl;

R is, at each occurrence, independently H or C₁-C₁₂ alkyl;

R¹ and R² are, at each occurrence, independently branched C₆-C₂₄ alkyl or branched C₆-C₂₄ alkenyl;

z is 0, 1 or 2, and

wherein each alkyl, alkenyl, alkylene, alkenylene, alkyleneoxide and alkenyleneoxide is independently substituted or unsubstituted unless otherwise specified.

In some embodiments of structure (VII), G³ is C₂-C₂₄ alkyleneoxide or C₂-C₂₄ alkenyleneoxide. In certain embodiments, G³ is unsubstituted. In other embodiments, G³ is substituted, for example substituted with hydroxyl. In more specific embodiments G³ is C₂-C₁₂ alkyleneoxide, for example, in some embodiments G³ is C₃-C₇ alkyleneoxide or in other embodiments G³ is C₃-C₁₂ alkyleneoxide.

In other embodiments of structure (VII), G³ is C₂-C₂₄ alkyleneaminyl or C₂-C₂₄ alkenyleneaminyl, for example C₆-C₁₂ alkyleneaminyl. In some of these embodiments, G³ is unsubstituted. In other of these embodiments, G³ is substituted with C₁-C₆ alkyl.

In some embodiments of structure (VII), X and X′ are each N, and Y and Y′ are each absent. In other embodiments, X and X′ are each CR, and Y and Y′ are each NR. In some of these embodiments, R is H.

In certain embodiments of structure (VII), X and X′ are each CR, and Y and Y′ are each independently —O(C═O)— or —(C═O)O—.

In some of the foregoing embodiments of structure (VII), the compound has one of the following structures (VIIA), (VIIB), (VIIC), (VIID), (VIIE), (VIIF), (VIIG) or (VIIH):

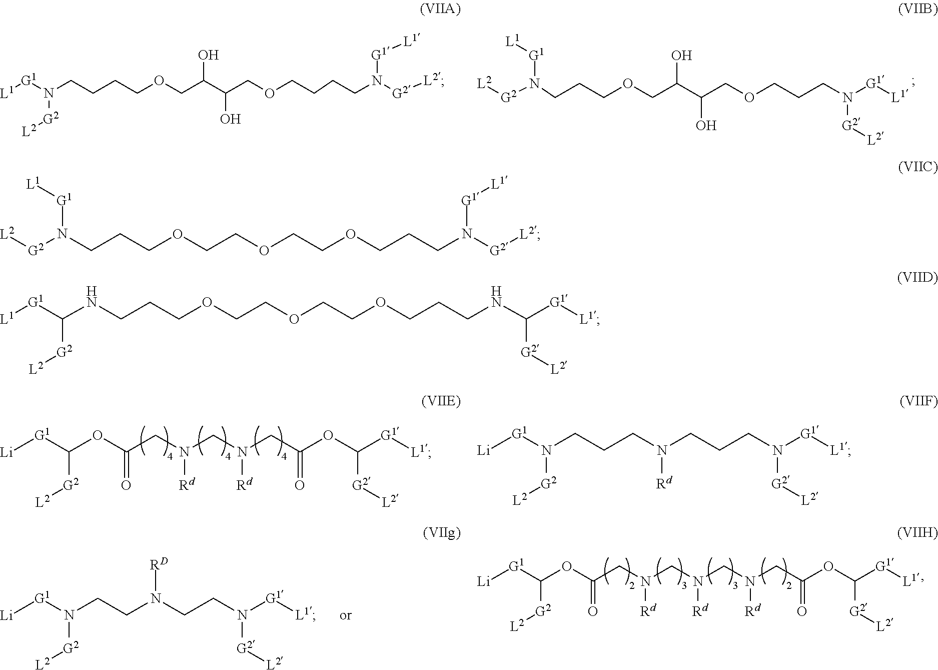

wherein R^{d} is, at each occurrence, independently H or optionally substituted C₁-C₆ alkyl. For example, in some embodiments R^{d} is H. In other embodiments, R^{d} is C₁-C₆ alkyl, such as methyl. In other embodiments, R^{d} is substituted C₁-C₆ alkyl, such as C₁-C₆ alkyl substituted with —O(C═O)R, —(C═O)OR, —NRC(═O)R or —C(═O)N(R)₂, wherein R is, at each occurrence, independently H or C₁-C₁₂ alkyl.

In some of the foregoing embodiments of structure (VII), L¹ and L^{1′} are each independently —O(C═O)R¹, —(C═O)OR¹ or —C(═O)NR^{b}R^{c}, and L² and L² are each independently —O(C═O)R², —(C═O)OR² or —C(═O)NR^{e}R^{f}. For example, in some embodiments L¹ and L^{1′} are each —(C═O)OR¹, and L² and L^{2′} are each —(C═O)OR². In other embodiments L¹ and L^{1′} are each —(C═O)OR¹, and L² and L^{2′} are each —C(═O)NR^{e}R^{f}. In other embodiments L¹ and L^{1′} are each —C(═O)NR^{b}R^{c}, and L² and L² are each —C(═O)NR^{e}R^{f}.

In some embodiments of the foregoing, G¹, G^{1′}, G² and G^{2′} are each independently C₂-C₈ alkylene, for example C₄-C₈ alkylene.

In some of the foregoing embodiments of structure (VII), R¹ or R², are each, at each occurrence, independently branched C₆-C₂₄ alkyl. For example, in some embodiments, R¹ and R² at each occurrence, independently have the following structure:

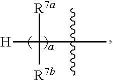

wherein:

R^{7a} and R^{7b} are, at each occurrence, independently H or C₁-C₁₂ alkyl; and

a is an integer from 2 to 12,

wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of structure (VII), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of structure (VII), R¹ or R², or both, at each occurrence independently has one of the following structures:

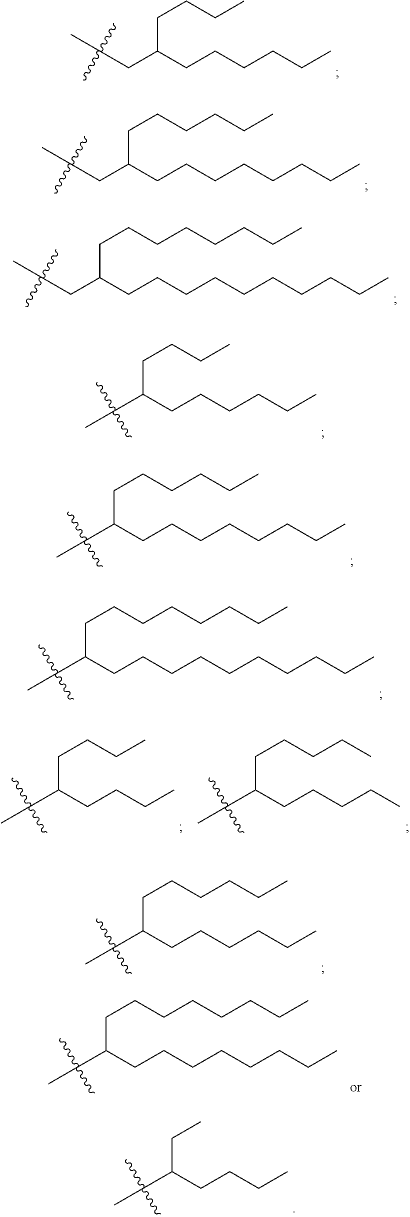

In some of the foregoing embodiments of structure (VII), R^{b}, R^{c}, R^{e} and R^{f}, when present, are each independently C₃-C₁₂ alkyl. For example, in some embodiments R^{b}, R^{c}, R^{e} and R^{f}, when present, are n-hexyl and in other embodiments R^{b}, R^{c}, R^{e} and R^{f}, when present, are n-octyl.

In various different embodiments of structure (VII), the cationic lipid has one of the structures set forth in Table 6 below.

TABLE 6
Representative cationic lipids of structure (VII)

| No. | Structure |
|---|---|
| VII-1 | 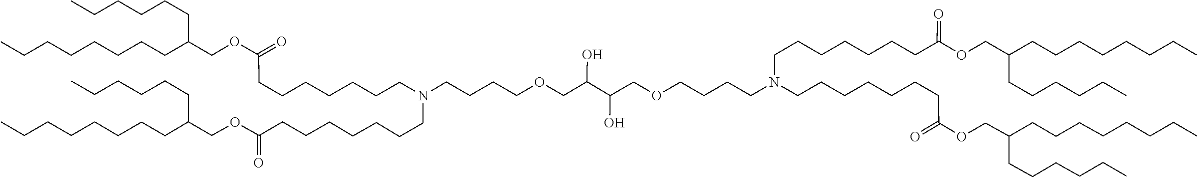 [SMILES: CCCCCCCCC(CCCCCC)COC(=O)CCCCCCCN(CCCCCCCC(=O)OCC(CCCCCC)CCCCCCCC)CCCCOCC(O)C(O)COCCCCN(CCCCCCCC(=O)OCC(CCCCCC)CCCCCCCC)CCCCCCCC(=O)OCC(CCCCCC)CCCCCCCC] |
| VII-2 | 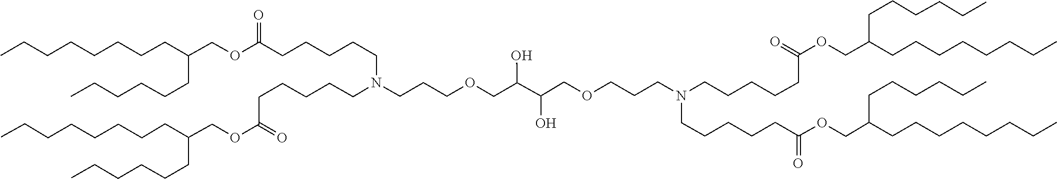 [SMILES: CCCCCCCCC(CCCCCC)COC(=O)CCCCCN(CCCCCC(=O)OCC(CCCCCC)CCCCCCCC)CCCOCC(O)C(O)COCCCN(CCCCCC(=O)OCC(CCCCCC)CCCCCCCC)CCCCCC(=O)OCC(CCCCCC)CCCCCCCC] |
| VII-3 | 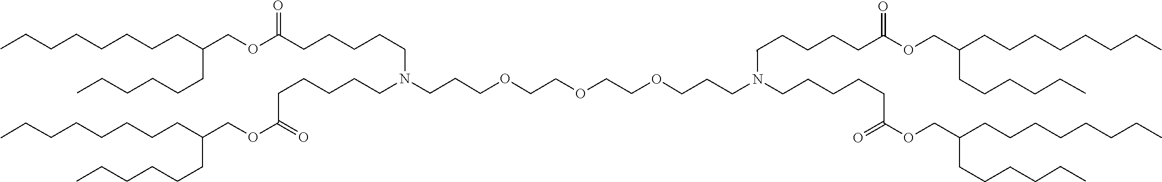 [SMILES: CCCCCCCCC(CCCCCC)COC(=O)CCCCCN(CCCCCC(=O)OCC(CCCCCC)CCCCCCCC)CCCOCCOCCOCCCN(CCCCCC(=O)OCC(CCCCCC)CCCCCCCC)CCCCCC(=O)OCC(CCCCCC)CCCCCCCC] |
| VII-4 | 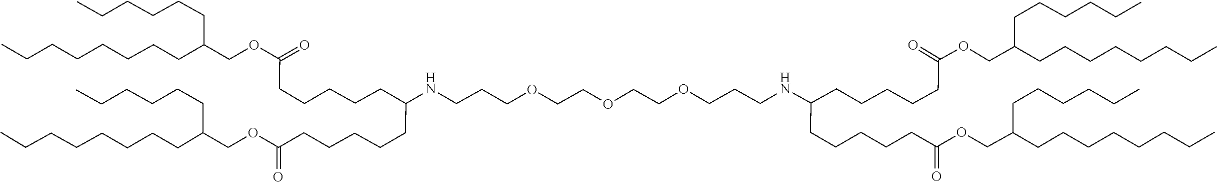 [SMILES: CCCCCCCCC(CCCCCC)COC(=O)CCCCCC(CCCCCC(=O)OCC(CCCCCC)CCCCCCCC)NCCCOCCOCCOCCCNC(CCCCCC(=O)OCC(CCCCCC)CCCCCCCC)CCCCCC(=O)OCC(CCCCCC)CCCCCCCC] |
| V1I-5 | 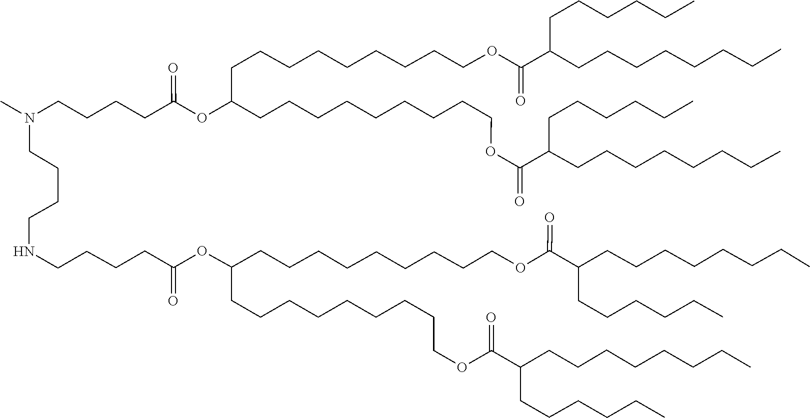 [SMILES: CCCCCCCCC(CCCCCC)C(=O)OCCCCCCCCCC(CCCCCCCCCOC(=O)C(CCCCCC)CCCCCCCC)OC(=O)CCCCNCCCCN(C)CCCCC(=O)OC(CCCCCCCCCOC(=O)C(CCCCCC)CCCCCCCC)CCCCCCCCCOC(=O)C(CCCCCC)CCCCCCCC] |
| VII-6 | 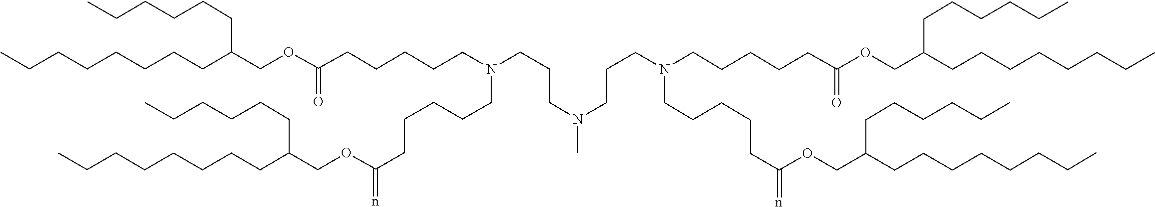 |
| VII-7 | 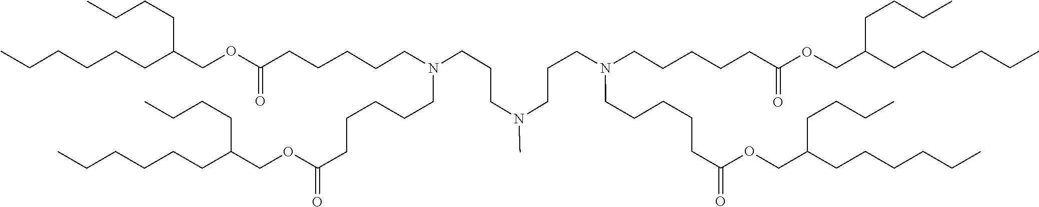 [SMILES: CCCCCCC(CCCC)COC(=O)CCCCCN(CCCCCC(=O)OCC(CCCC)CCCCCC)CCCN(C)CCCN(CCCCCC(=O)OCC(CCCC)CCCCCC)CCCCCC(=O)OCC(CCCC)CCCCCC] |
| VII-8 | 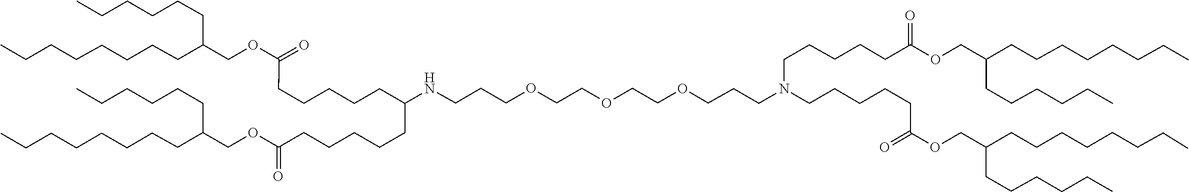 [SMILES: CCCCCCCCC(CCCCCC)COC(=O)CCCCCC(CCCCCC(=O)OCC(CCCCCC)CCCCCCCC)NCCCOCCOCCOCCCN(CCCCCC(=O)OCC(CCCCCC)CCCCCCCC)CCCCCC(=O)OCC(CCCCCC)CCCCCCCC] |
| VII-9 | 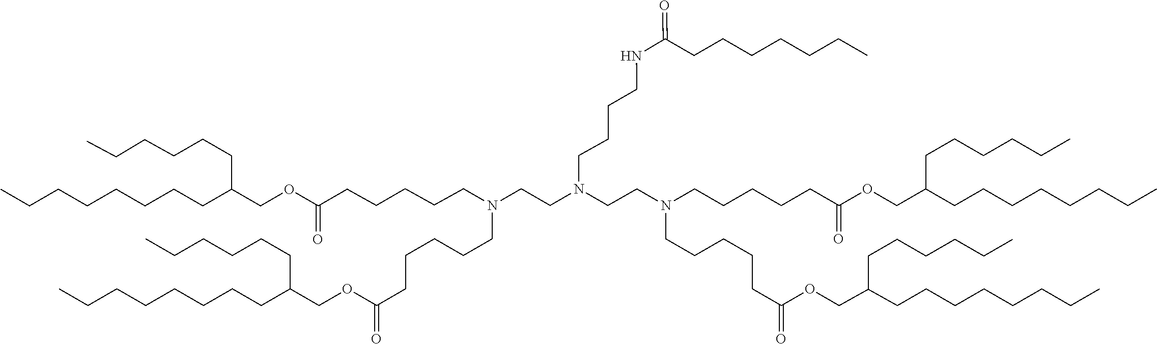 [SMILES: CCCCCCCCC(CCCCCC)COC(=O)CCCCCN(CCCCCC(=O)OCC(CCCCCC)CCCCCCCC)CCN(CCCCNC(=O)CCCCCCC)CCN(CCCCCC(=O)OCC(CCCCCC)CCCCCCCC)CCCCCC(=O)OCC(CCCCCC)CCCCCCCC] |
| VII-10 | 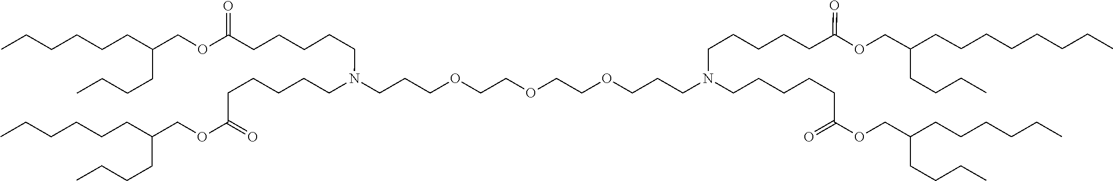 [SMILES: CCCCCCCCC(CCCC)COC(=O)CCCCCN(CCCCCC(=O)OCC(CCCC)CCCCCC)CCCOCCOCCOCCCN(CCCCCC(=O)OCC(CCCC)CCCCCC)CCCCCC(=O)OCC(CCCC)CCCCCC] |
| VII-11 | 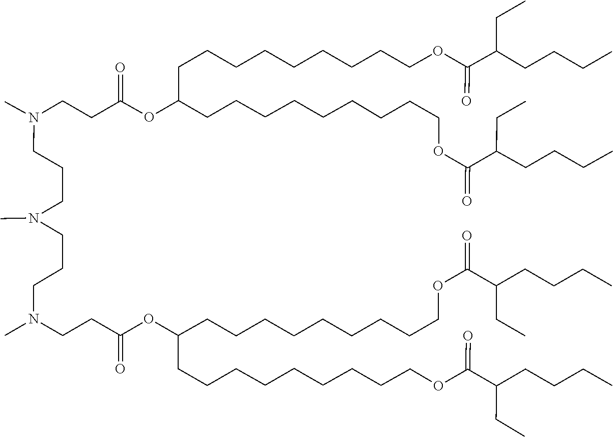 [SMILES: CCCCC(CC)C(=O)OCCCCCCCCCC(CCCCCCCCCOC(=O)C(CC)CCCC)OC(=O)CCN(C)CCCN(C)CCCN(C)CCC(=O)OC(CCCCCCCCCOC(=O)C(CC)CCCC)CCCCCCCCCOC(=O)C(CC)CCCC] |

In one embodiment, the cationic lipid is a compound having the following structure (VIII):

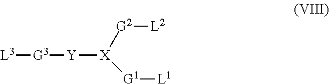

or a pharmaceutically acceptable salt, prodrug or stereoisomer thereof, wherein:

X is N, and Y is absent; or X is CR, and Y is NR;

L¹ is —O(C═O)R¹, —(C═O)OR¹, —C(═O)R¹, —S(O)—R¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹,

L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;

L³ is —O(C═O)R³ or —(C═O)OR³;

G¹ and G² are each independently C₂-C₁₂ alkylene or C₂-C₁₂ alkenylene;

G³ is C₁-C₂₄ alkylene, C₂-C₂₄ alkenylene, C₁-C₂₄ heteroalkylene or C₂-C₂₄heteroalkenylene;

R^{a}, R^{b}, R^{d} and R^{e} are each independently H or C₁-C₁₂ alkyl or C₁-C₁₂ alkenyl;

R^{e} and R^{f} are each independently C₁-C₁₂ alkyl or C₂-C₁₂ alkenyl;

each R is independently H or C₁-C₁₂ alkyl;

R¹, R² and R³ are each independently C₁-C₂₄ alkyl or C₂-C₂₄ alkenyl; and

x is 0, 1 or 2, and

wherein each alkyl, alkenyl, alkylene, alkenylene, heteroalkylene and heteroalkenylene is independently substituted or unsubstituted unless otherwise specified.

In more embodiments of structure (I):

X is N, and Y is absent; or X is CR, and Y is NR;

L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;

L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;

L³ is —O(C═O)R³ or —(C═O)OR³;

G¹ and G² are each independently C₂-C₁₂ alkylene or C₂-C₁₂ alkenylene;

G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;

R^{a}, R^{b}, R^{d} and R^{e} are each independently H or C₁-C₁₂ alkyl or C₁-C₁₂ alkenyl;

R^{e} and R^{f} are each independently C₁-C₁₂ alkyl or C₂-C₁₂ alkenyl;

each R is independently H or C₁-C₁₂ alkyl;

R¹, R² and R³ are each independently C₁-C₂₄ alkyl or C₂-C₂₄ alkenyl; and

x is 0, 1 or 2, and

wherein each alkyl, alkenyl, alkylene, alkenylene, heteroalkylene and heteroalkenylene is independently substituted or unsubstituted unless otherwise specified.

In other embodiments of structure (I):

X is N and Y is absent, or X is CR and Y is NR,

L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;

L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;

L³ is —O(C═O)R³ or —(C═O)OR³;

G¹ and G² are each independently C₂-C₁₂ alkylene or C₂-C₁₂ alkenylene;

G³ is C₁-C₂₄ alkylene, C₂-C₂₄ alkenylene, C₁-C₂₄ heteroalkylene or C₂-C₂₄ heteroalkenylene;

R^{a}, R^{b}, R^{d} and R^{e} are each independently H or C₁-C₁₂ alkyl or C₁-C₁₂ alkenyl;

R^{c} and R^{f} are each independently C₁-C₁₂ alkyl or C₂-C₁₂ alkenyl;

each R is independently H or C₁-C₁₂ alkyl;

R¹, R² and R³ are each independently branched C₆-C₂₄ alkyl or branched C₆-C₂₄ alkenyl; and

x is 0, 1 or 2, and

wherein each alkyl, alkenyl, alkylene, alkenylene, heteroalkylene and heteroalkenylene is independently substituted or unsubstituted unless otherwise specified.

In certain embodiments of structure (VIII), G³ is unsubstituted. In more specific embodiments G³ is C₂-C₁₂ alkylene, for example, in some embodiments G³ is C₃-C₇ alkylene or in other embodiments G³ is C₃-C₁₂ alkylene. In some embodiments, G³ is C₂ or C₃ alkylene.

In other embodiments of structure (VIII), G³ is C₁-C₁₂ heteroalkylene, for example C₁-C₁₂ aminylalkylene.

In certain embodiments of structure (VIII), X is N and Y is absent. In other embodiments, X is CR and Y is NR, for example in some of these embodiments R is H.

In some of the foregoing embodiments of structure (VIII), the compound has one of the following structures (VIIIA), (VIIIB), (VIIIC) or (VIIID):

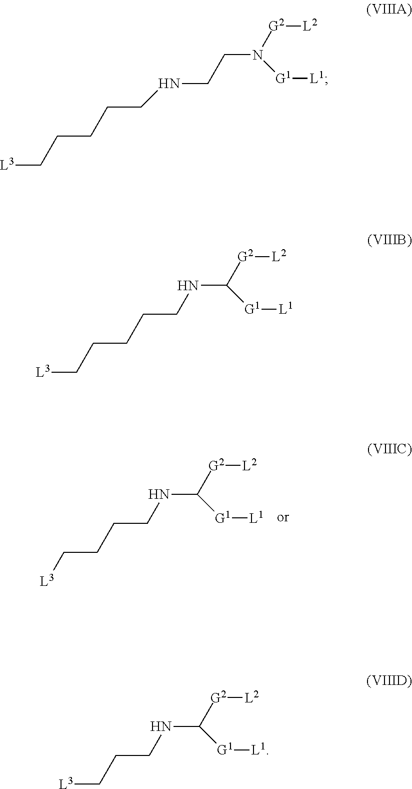

In some of the foregoing embodiments of structure (VIII), L¹ is —O(C═O)R¹, —(C═O)OR¹ or —C(═O)NR^{b}R^{c}, and L² is —O(C═O)R², —(C═O)OR² or —C(═O)NR^{e}R^{f}. In other specific embodiments, L¹ is —(C═O)OR¹ and L² is —(C═O)OR². In any of the foregoing embodiments, L³ is —(C═O)OR³.

In some of the foregoing embodiments of structure (VIII), G¹ and G² are each independently C₂-C₁₂ alkylene, for example C₄-C₁₀ alkylene.

In some of the foregoing embodiments of structure (VIII), R¹, R² and R³ are each, independently branched C₆-C₂₄ alkyl. For example, in some embodiments, R¹, R² and R³ each, independently have the following structure:

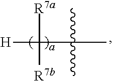

wherein:

R^{7a} and R^{7b} are, at each occurrence, independently H or C₁-C₁₂ alkyl;

and

a is an integer from 2 to 12,

wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of structure (VIII), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In some of the foregoing embodiments of structure (VIII), X is CR, Y is NR and R³ is C₁-C₁₂ alkyl, such as ethyl, propyl or butyl. In some of these embodiments, le and R² are each independently branched C₆-C₂₄ alkyl.

In different embodiments of structure (VIII), R¹, R² and R³ each, independently have one of the following structures:

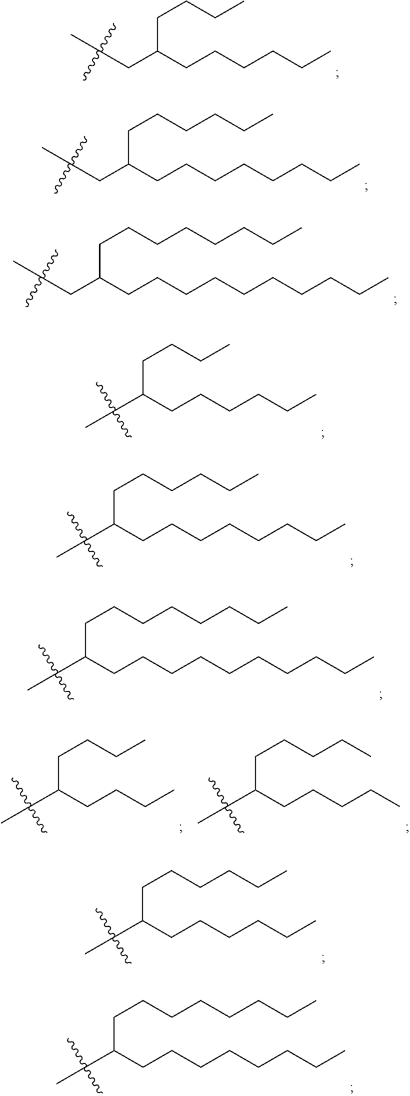

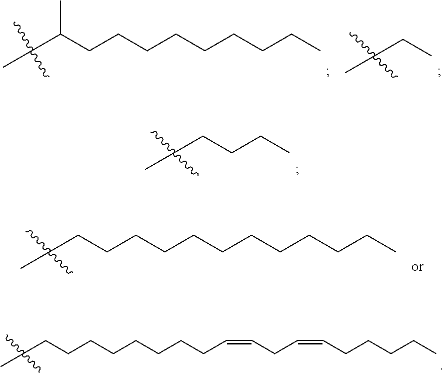

In certain embodiments of structure (VIII), R¹ and R² and R³ are each, independently, branched C₆-C₂₄ alkyl and R³ is C₁-C₂₄ alkyl or C₂-C₂₄ alkenyl.

In some of the foregoing embodiments of structure (VIII), R^{b}, R^{c}, R^{e} and R^{f} are each independently C₃-C₁₂ alkyl. For example, in some embodiments R^{b}, R^{c}, R^{e} and R^{f} are n-hexyl and in other embodiments R^{b}, R^{c}, R^{e} and R^{f} are n-octyl.

In various different embodiments of structure (VIII), the compound has one of the structures set forth in Table 7 below.

TABLE 7
Representative cationic lipids of structure (VIII)

| No. | Structure |
|---|---|
| VIII-1 | 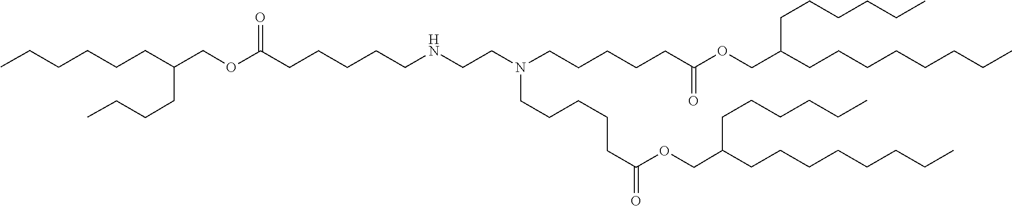 [SMILES: CCCCCCCCC(CCCCCC)COC(=O)CCCCCN(CCCCCC(=O)OCC(CCCCCC)CCCCCCCC)CCNCCCCCC(=O)OCC(CCCC)CCCCCC] |
| VIII-2 | 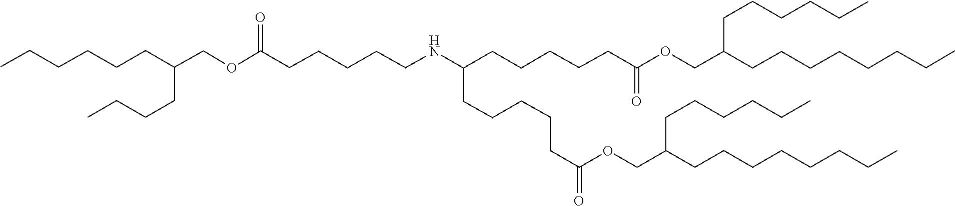 [SMILES: CCCCCCCCC(CCCCCC)COC(=O)CCCCCC(CCCCCC(=O)OCC(CCCCCC)CCCCCCCC)NCCCCCC(=O)OCC(CCCC)CCCCCC] |
| VIII-3 | 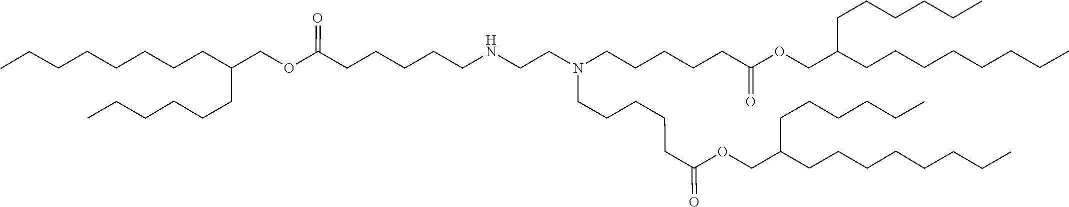 [SMILES: CCCCCCCCC(CCCCCC)COC(=O)CCCCCNCCN(CCCCCC(=O)OCC(CCCCCC)CCCCCCCC)CCCCCC(=O)OCC(CCCCCC)CCCCCCCC] |
| VIII-4 | 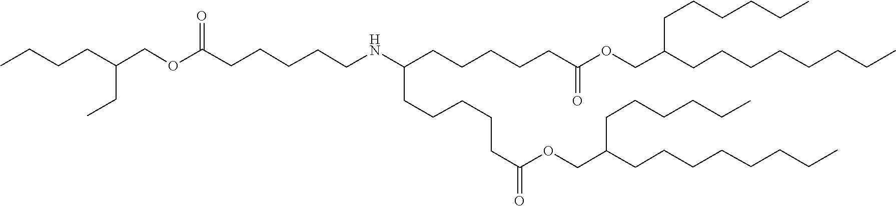 [SMILES: CCCCCCCCC(CCCCCC)COC(=O)CCCCCC(CCCCCC(=O)OCC(CCCCCC)CCCCCCCC)NCCCCCC(=O)OCC(CC)CCCC] |
| VIII-5 | 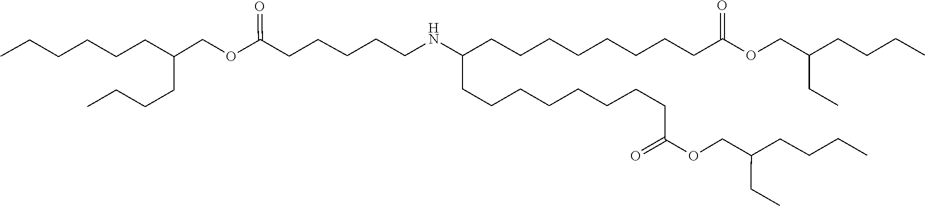 [SMILES: CCCCCCC(CCCC)COC(=O)CCCCCNC(CCCCCCCCC(=O)OCC(CC)CCCC)CCCCCCCCC(=O)OCC(CC)CCCC] |
| VIII-6 | 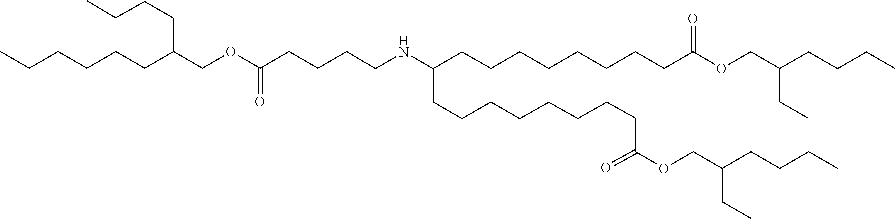 [SMILES: CCCCCCC(CCCC)COC(=O)CCCCNC(CCCCCCCCC(=O)OCC(CC)CCCC)CCCCCCCCC(=O)OCC(CC)CCCC] |
| VIII-7 | 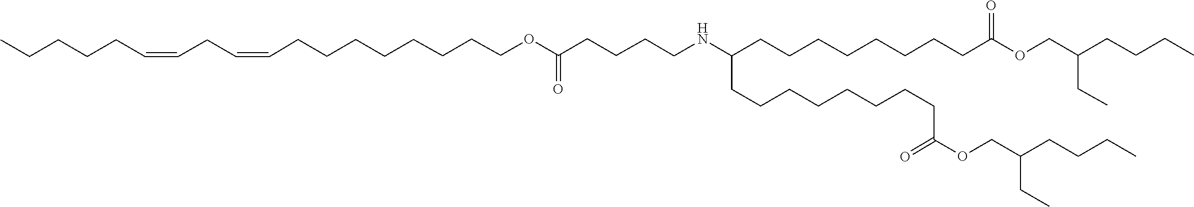 [SMILES: CCCCC/C=C\C/C=C\CCCCCCCCOC(=O)CCCCNC(CCCCCCCCC(=O)OCC(CC)CCCC)CCCCCCCCC(=O)OCC(CC)CCCC] |
| VIII-8 | 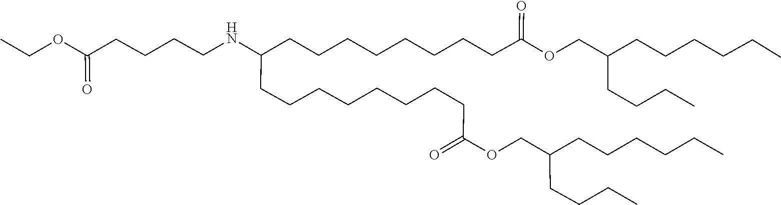 [SMILES: CCCCCCC(CCCC)COC(=O)CCCCCCCCC(CCCCCCCCC(=O)OCC(CCCC)CCCCCC)NCCCCC(=O)OCC] |
| VIII-9 | 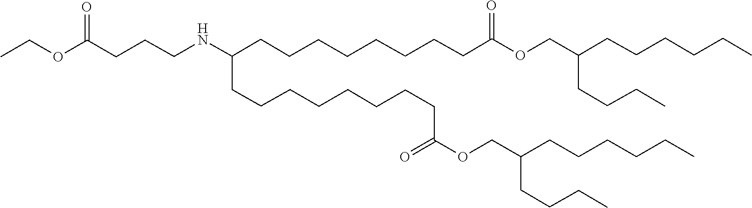 [SMILES: CCCCCCC(CCCC)COC(=O)CCCCCCCCC(CCCCCCCCC(=O)OCC(CCCC)CCCCCC)NCCCC(=O)OCC] |
| VIII-10 | 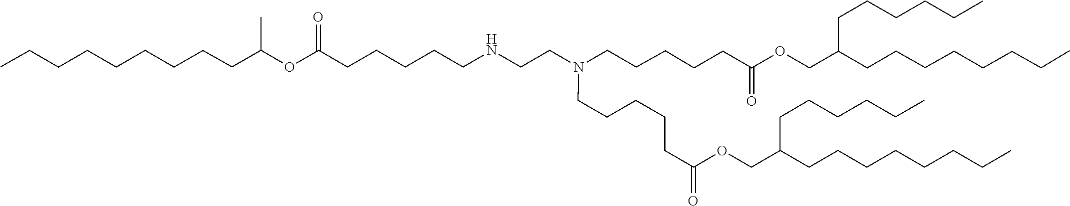 [SMILES: CCCCCCCCCC(C)OC(=O)CCCCCNCCN(CCCCCC(=O)OCC(CCCCCC)CCCCCCCC)CCCCCC(=O)OCC(CCCCCC)CCCCCCCC] |
| VIII-11 | 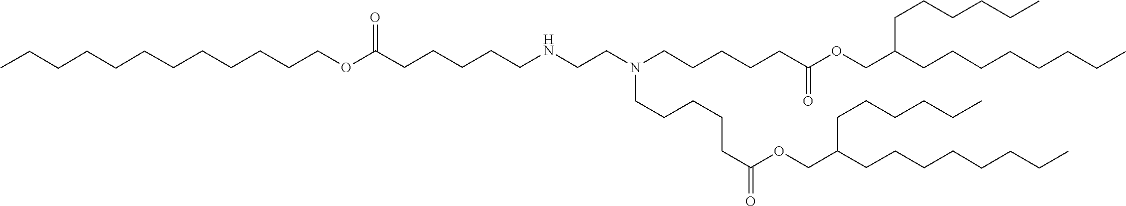 [SMILES: CCCCCCCCCCCCOC(=O)CCCCCNCCN(CCCCCC(=O)OCC(CCCCCC)CCCCCCCC)CCCCCC(=O)OCC(CCCCCC)CCCCCCCC] |
| VIII-12 | 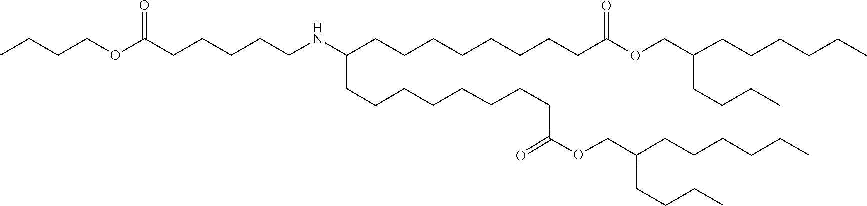 [SMILES: CCCCCCC(CCCC)COC(=O)CCCCCCCCC(CCCCCCCCC(=O)OCC(CCCC)CCCCCC)NCCCCCC(=O)OCCCC] |

In one embodiment, the cationic lipid is a compound having the following structure (IX):

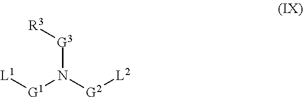

or a pharmaceutically acceptable salt, prodrug or stereoisomer thereof, wherein:

L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;

L² is —O(C═O)R², —(C═O)OR², —C(═O)R², —OR², —S(O),R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR² or a direct bond to R²;

G¹ and G² are each independently C₂-C₁₂ alkylene or C₂-C₁₂ alkenylene;

G³ is C₁-C₂₄ alkylene, C₂-C₂₄ alkenylene, C₃-C₈ cycloalkylene or C₃-C₈ cycloalkenylene;

R^{a}, R^{b}, R^{d} and R^{e} are each independently H or C₁-C₁₂ alkyl or C₁-C₁₂ alkenyl;

R^{e} and R^{f} are each independently C₁-C₁₂ alkyl or C₂-C₁₂ alkenyl;

R¹ and R² are each independently branched C₆-C₂₄ alkyl or branched C₆-C₂₄ alkenyl;

R³ is —N(R⁴)R⁵;

R⁴ is C₁-C₁₂ alkyl;

R⁵ is substituted C₁-C₁₂ alkyl; and

x is 0, 1 or 2, and

wherein each alkyl, alkenyl, alkylene, alkenylene, cycloalkylene, cycloalkenylene, aryl and aralkyl is independently substituted or unsubstituted unless otherwise specified.

In certain embodiments of structure (XI), G³ is unsubstituted. In more specific embodiments G³ is C₂-C₁₂ alkylene, for example, in some embodiments G³ is C₃-C₇ alkylene or in other embodiments G³ is C₃-C₁₂ alkylene. In some embodiments, G³ is C₂ or C₃ alkylene.

In some of the foregoing embodiments of structure (IX), the compound has the following structure (IX A):

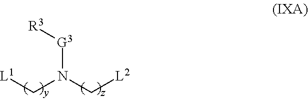

wherein y and z are each independently integers ranging from 2 to 12, for example an integer from 2 to 6, from 4 to 10, or for example 4 or 5. In certain embodiments, y and z are each the same and selected from 4, 5, 6, 7, 8 and 9.

In some of the foregoing embodiments of structure (IX), L¹ is —O(C═O)R¹, —(C═O)OR¹ or —C(═O)NR^{b}R^{c}, and L² is —O(C═O)R², —(C═O)OR² or —C(═O)NR^{e}R^{f}. For example, in some embodiments L¹ and L² are —(C═O)OR¹ and —(C═O)OR², respectively. In other embodiments L¹ is —(C═O)OR¹ and L² is —C(═O)NR^{e}R^{f}. In other embodiments L¹ is

—C(═O)NR^{b}R^{c} and L² is —C(═O)NR^{e}R^{f}.

In other embodiments of the foregoing, the compound has one of the following structures (IXB), (IXC), (IXD) or (IXE):

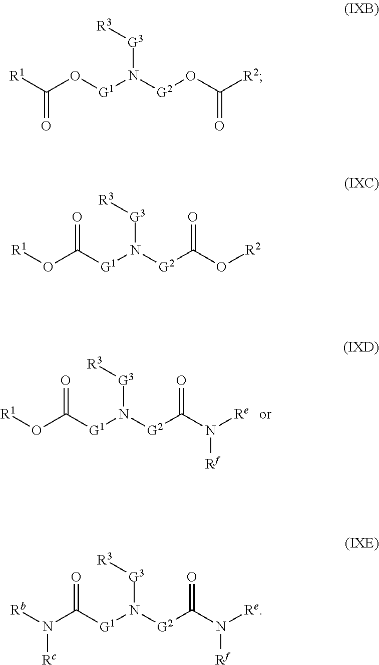

In some of the foregoing embodiments, the compound has structure (IXB), in other embodiments, the compound has structure (IXC) and in still other embodiments the compound has the structure (IXD). In other embodiments, the compound has structure (IXE).

In some different embodiments of the foregoing, the compound has one of the following structures (IXF), (IXG), (IXH) or (IXJ):

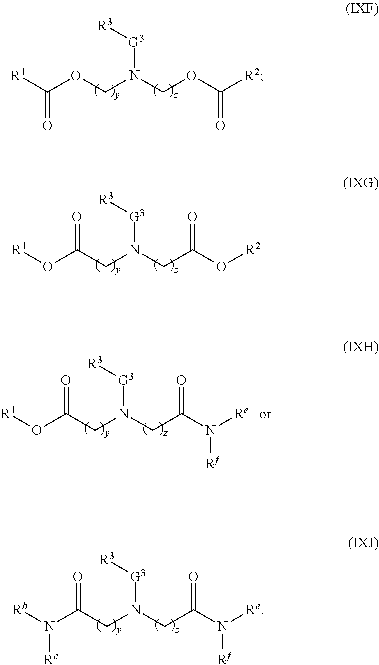

wherein y and z are each independently integers ranging from 2 to 12, for example an integer from 2 to 6, for example 4.

In some of the foregoing embodiments of structure (IX), y and z are each independently an integer ranging from 2 to 10, 2 to 8, from 4 to 10 or from 4 to 7. For example, in some embodiments, y is 4, 5, 6, 7, 8, 9, 10, 11 or 12. In some embodiments, z is 4, 5, 6, 7, 8, 9, 10, 11 or 12. In some embodiments, y and z are the same, while in other embodiments y and z are different.

In some of the foregoing embodiments of structure (IX), R¹ or R², or both is branched C₆-C₂₄ alkyl. For example, in some embodiments, R¹ and R² each, independently have the following structure:

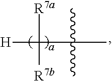

wherein:

R^{7a} and R^{7b} are, at each occurrence, independently H or C₁-C₁₂ alkyl; and

a is an integer from 2 to 12,

wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of structure (IX), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of structure (IX), R¹ or R², or both, has one of the following structures:

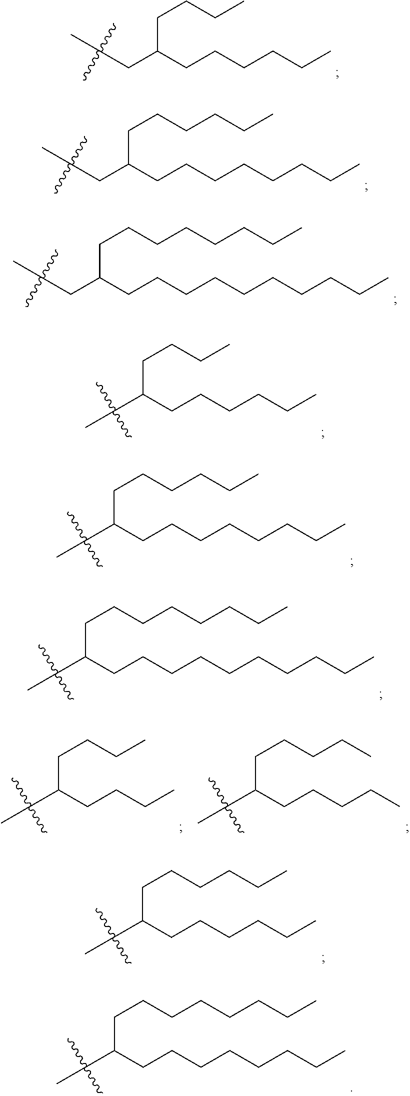

In some of the foregoing embodiments of structure (IX), R^{b}, R^{c}, R^{e} and R^{f} are each independently C₃-C₁₂ alkyl. For example, in some embodiments R^{b}, R^{c}, R^{e} and R^{f} are n-hexyl and in other embodiments R^{b}, R^{c}, R^{e} and R^{f} are n-octyl.

In any of the foregoing embodiments of structure (IX), R⁴ is substituted or unsubstituted: methyl, ethyl, propyl, n-butyl, n-hexyl, n-octyl or n-nonyl. For example, in some embodiments R⁴ is unsubstituted. In other R⁴ is substituted with one or more substituents selected from the group consisting of —OR^{g}, —NR^{g}C(═O)R^{h}, —C(═O)NR^{g}R^{h}, —C(═O)R^{h}, —OC(═O)R^{h}, —C(═O)OR^{h} and —ORⁱOH, wherein:

R^{g} is, at each occurrence independently H or C₁-C₆ alkyl;

R^{h} is at each occurrence independently C₁-C₆ alkyl; and

Rⁱ is, at each occurrence independently C₁-C₆ alkylene.

In other of the foregoing embodiments of structure (IX), R⁵ is substituted: methyl, ethyl, propyl, n-butyl, n-hexyl, n-octyl or n-nonyl. In some embodiments, R⁵ is substituted ethyl or substituted propyl. In other different embodiments, R⁵ is substituted with hydroxyl. In still more embodiments, R⁵ is substituted with one or more substituents selected from the group consisting of —OR^{g}, —NR^{g}C(═O)R^{h}, —C(═O)NR^{g}R^{h}, —C(═O)R^{h}, —OC(═O)R^{h}, —C(═O)OR^{h} and —ORⁱOH, wherein:

R^{g} is, at each occurrence independently H or C₁-C₆ alkyl;

R^{h} is at each occurrence independently C₁-C₆ alkyl; and

Rⁱ is, at each occurrence independently C₁-C₆ alkylene.

In other embodiments of structure (IX), R⁴ is unsubstituted methyl, and R⁵ is substituted: methyl, ethyl, propyl, n-butyl, n-hexyl, n-octyl or n-nonyl. In some of these embodiments, R⁵ is substituted with hydroxyl.

In some other specific embodiments of structure (IX), R³ has one of the following structures:

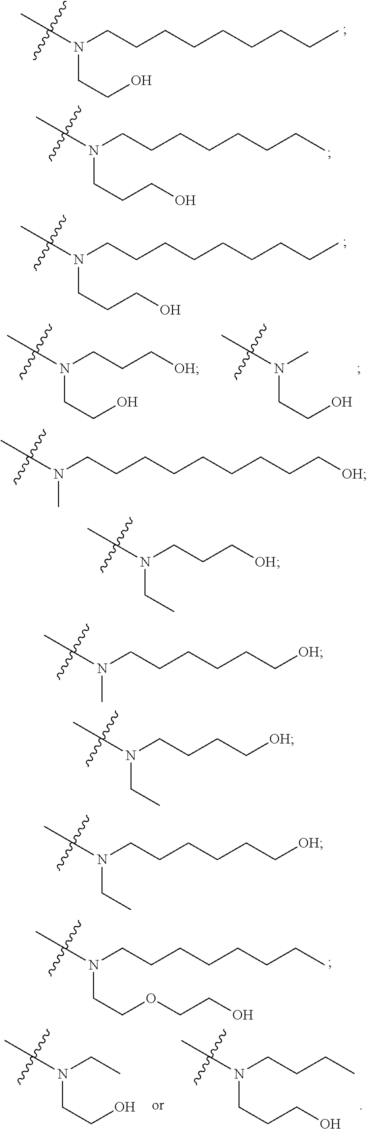

In various different embodiments of structure (IX), the cationic lipid has one of the structures set forth in Table 8 below.

TABLE 8
Representative cationic lipids of structure (IX)

| No. | Structure |
|---|---|
| IX-1 | 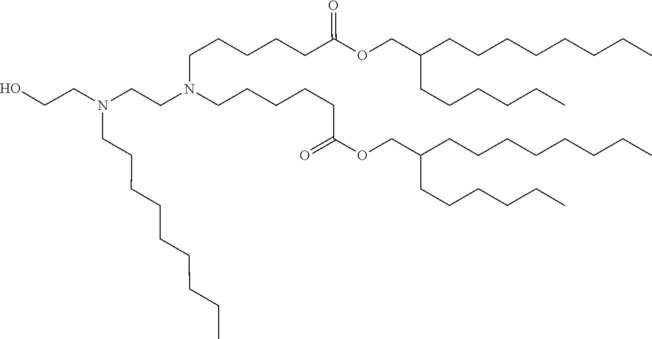 [SMILES: CCCCCCCCCN(CCO)CCN(CCCCCC(=O)OCC(CCCCCC)CCCCCCCC)CCCCCC(=O)OCC(CCCCCC)CCCCCCCC] |
| IX-2 | 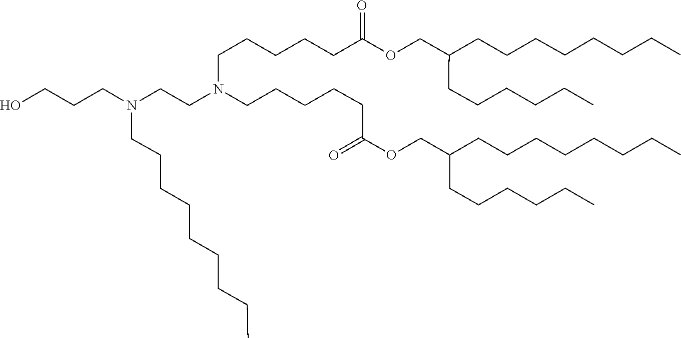 [SMILES: CCCCCCCCCN(CCCO)CCN(CCCCCC(=O)OCC(CCCCCC)CCCCCCCC)CCCCCC(=O)OCC(CCCCCC)CCCCCCCC] |
| IX-3 | 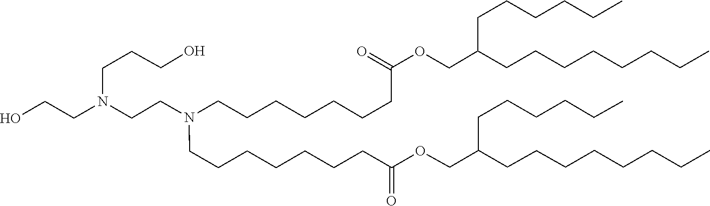 [SMILES: CCCCCCCCC(CCCCCC)COC(=O)CCCCCCCN(CCCCCCCC(=O)OCC(CCCCCC)CCCCCCCC)CCN(CCO)CCCO] |
| IX-4 | 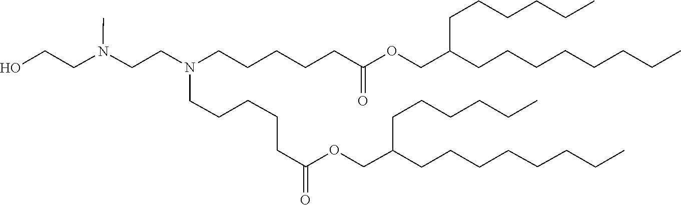 [SMILES: CCCCCCCCC(CCCCCC)COC(=O)CCCCCN(CCCCCC(=O)OCC(CCCCCC)CCCCCCCC)CCN(C)CCO] |
| IX-5 | 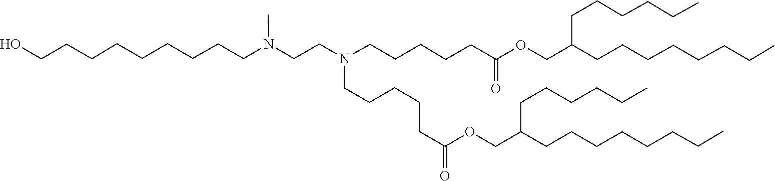 [SMILES: CCCCCCCCC(CCCCCC)COC(=O)CCCCCN(CCCCCC(=O)OCC(CCCCCC)CCCCCCCC)CCN(C)CCCCCCCCCO] |
| IX-6 | 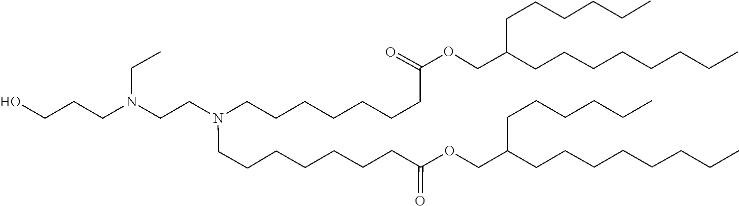 [SMILES: CCCCCCCCC(CCCCCC)COC(=O)CCCCCCCN(CCCCCCCC(=O)OCC(CCCCCC)CCCCCCCC)CCN(CC)CCCO] |
| IX-7 | 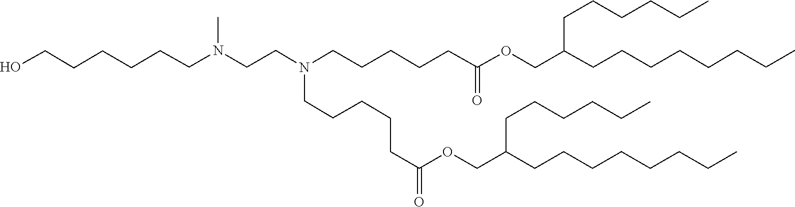 [SMILES: CCCCCCCCC(CCCCCC)COC(=O)CCCCCN(CCCCCC(=O)OCC(CCCCCC)CCCCCCCC)CCN(C)CCCCCCO] |
| IX-8 | 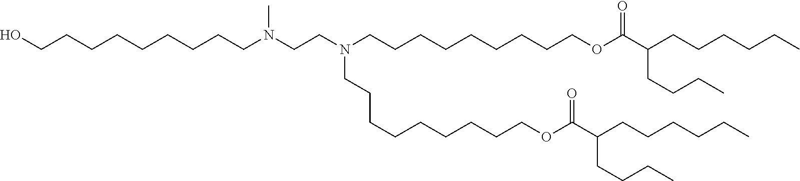 [SMILES: CCCCCCC(CCCC)C(=O)OCCCCCCCCCN(CCCCCCCCCOC(=O)C(CCCC)CCCCCC)CCN(C)CCCCCCCCCO] |
| IX-9 | 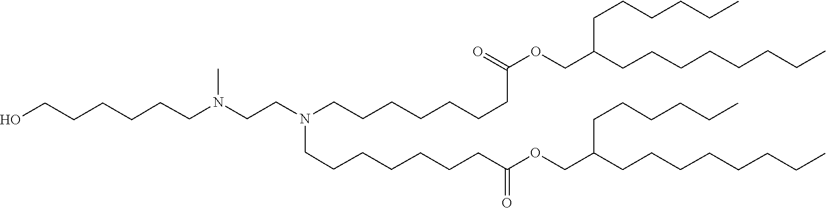 [SMILES: CCCCCCCCC(CCCCCC)COC(=O)CCCCCCCN(CCCCCCCC(=O)OCC(CCCCCC)CCCCCCCC)CCN(C)CCCCCCO] |
| IX-10 | 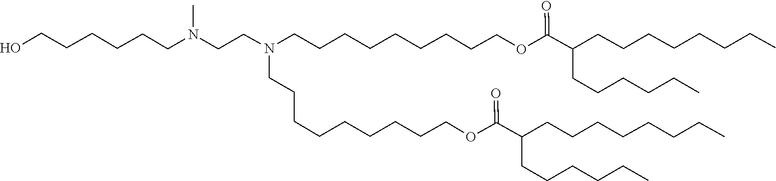 [SMILES: CCCCCCCCC(CCCCCC)C(=O)OCCCCCCCCCN(CCCCCCCCCOC(=O)C(CCCCCC)CCCCCCCC)CCN(C)CCCCCCO] |
| IX-11 | 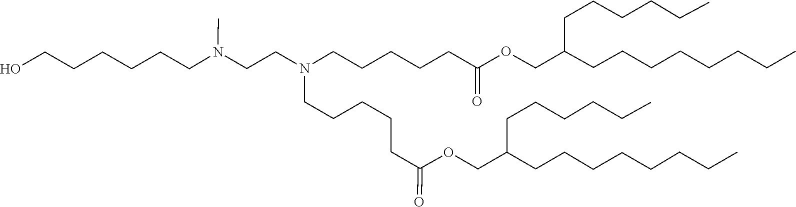 [SMILES: CCCCCCCCC(CCCCCC)COC(=O)CCCCCN(CCCCCC(=O)OCC(CCCCCC)CCCCCCCC)CCN(C)CCCCCCO] |
| IX-12 | 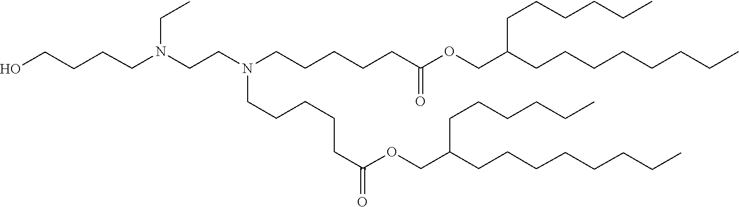 [SMILES: CCCCCCCCC(CCCCCC)COC(=O)CCCCCN(CCCCCC(=O)OCC(CCCCCC)CCCCCCCC)CCN(CC)CCCCO] |
| IX-13 | 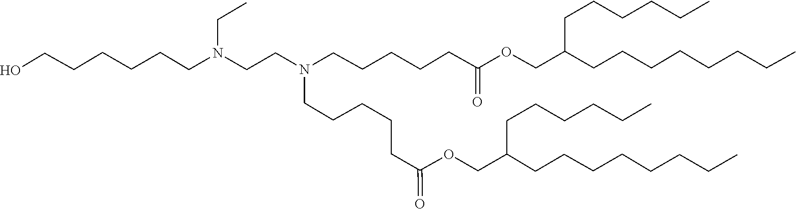 [SMILES: CCCCCCCCC(CCCCCC)COC(=O)CCCCCN(CCCCCC(=O)OCC(CCCCCC)CCCCCCCC)CCN(CC)CCCCCCO] |
| IX-14 | 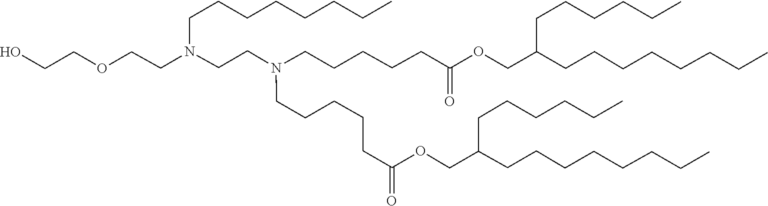 [SMILES: CCCCCCCCC(CCCCCC)COC(=O)CCCCCN(CCCCCC(=O)OCC(CCCCCC)CCCCCCCC)CCN(CCCCCCCC)CCOCCO] |
| IX-15 | 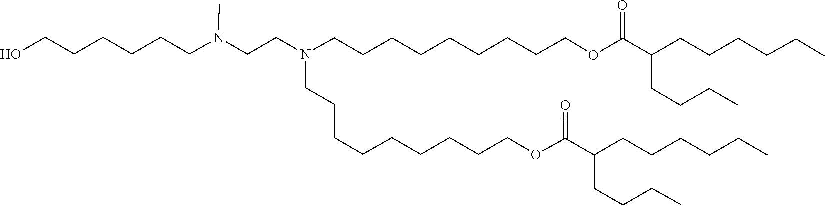 [SMILES: CCCCCCC(CCCC)C(=O)OCCCCCCCCCN(CCCCCCCCCOC(=O)C(CCCC)CCCCCC)CCN(C)CCCCCCO] |
| IX-16 | 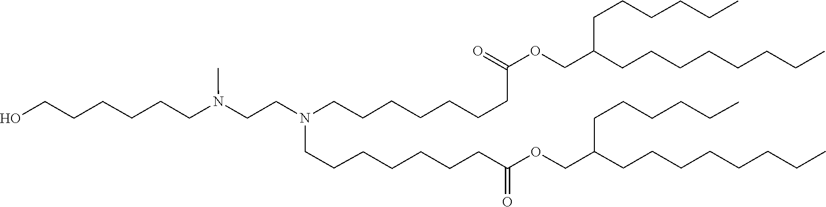 [SMILES: CCCCCCCCC(CCCCCC)COC(=O)CCCCCCCN(CCCCCCCC(=O)OCC(CCCCCC)CCCCCCCC)CCN(C)CCCCCCO] |
| IX-17 | 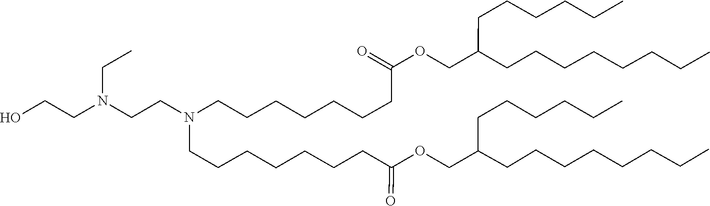 [SMILES: CCCCCCCCC(CCCCCC)COC(=O)CCCCCCCN(CCCCCCCC(=O)OCC(CCCCCC)CCCCCCCC)CCN(CC)CCO] |
| IX-18 | 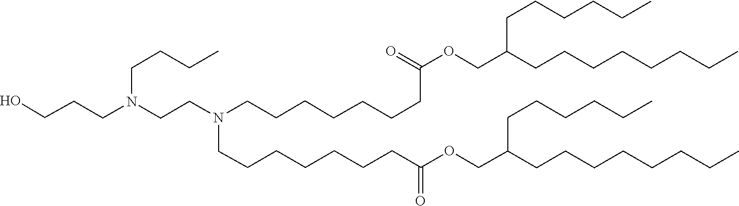 [SMILES: CCCCCCCCC(CCCCCC)COC(=O)CCCCCCCN(CCCCCCCC(=O)OCC(CCCCCC)CCCCCCCC)CCN(CCCC)CCCO] |

In one embodiment, the cationic lipid is a compound having the following structure (X):

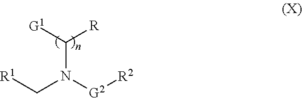

or a pharmaceutically acceptable salt, tautomer, prodrug or stereoisomer thereof, wherein:

G¹ is —OH, —NR³R⁴, —(C═O)NR⁵ or —NR³(C═O)R⁵;

G² is —CH₂— or —(C═O)—;

R is, at each occurrence, independently H or OH;

R¹ and R² are each independently branched, saturated or unsaturated C₁₂-C₃₆ alkyl;

R³ and R⁴ are each independently H or straight or branched, saturated or unsaturated C₁-C₆ alkyl;

R⁵ is straight or branched, saturated or unsaturated C₁-C₆ alkyl; and

n is an integer from 2 to 6.

In some embodiments, R¹ and R² are each independently branched, saturated or unsaturated C₁₂-C₃₀ alkyl, C₁₂-C₂₀ alkyl, or C₁₅-C₂₀ alkyl. In some specific embodiments, R¹ and R² are each saturated. In certain embodiments, at least one of R¹ and R² is unsaturated.

In some of the foregoing embodiments of structure (X), R¹ and R² have the following structure:

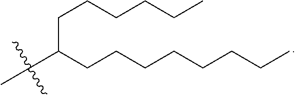 [SMILES: *C(CCCCCC)CCCCCCCC]

In some of the foregoing embodiments of structure (X), the compound has the following structure (XA):

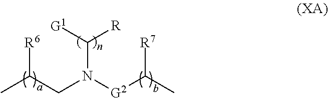

wherein:

R⁶ and R⁷ are, at each occurrence, independently H or straight or branched, saturated or unsaturated C₁-C₁₄ alkyl;

a and b are each independently an integer ranging from 1 to 15,

provided that R⁶ and a, and R⁷ and b, are each independently selected such that R¹ and R², respectively, are each independently branched, saturated or unsaturated C₁₂-C₃₆ alkyl.

In some of the foregoing embodiments, the compound has the following structure (XB):

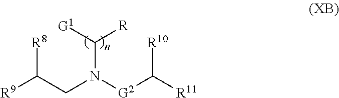

wherein:

R⁸, R⁹, R¹⁰ and R¹¹ are each independently straight or branched, saturated or unsaturated C₄-C₁₂ alkyl, provided that R⁸ and R⁹, and R¹⁰ and R¹¹, are each independently selected such that R¹ and R², respectively, are each independently branched, saturated or unsaturated C₁₂-C₃₆ alkyl. In some embodiments of (XB), R⁸, R⁹, R¹⁰ and R¹¹ are each independently straight or branched, saturated or unsaturated C₆-C₁₀ alkyl. In certain embodiments of (XB), at least one of R⁸, R⁹, R¹⁰ and R¹¹ is unsaturated. In other certain specific embodiments of (XB), each of R⁸, R⁹, R¹⁰ and R¹¹ is saturated.

In some of the foregoing embodiments, the compound has structure (XA), and in other embodiments, the compound has structure (XB).

In some of the foregoing embodiments, G¹ is —OH, and in some embodiments G¹ is —NR³R⁴. For example, in some embodiments, G¹ is —NH₂, —NHCH₃ or —N(CH₃)₂. In certain embodiments, G¹ is —(C═O)NR⁵. In certain other embodiments, G¹ is —NR³(C═O)R⁵. For example, in some embodiments G¹ is —NH(C═O)CH₃ or —NH(C═O)CH₂CH₂CH₃.

In some of the foregoing embodiments of structure (X), G² is —CH₂—. In some different embodiments, G² is —(C═O)—.

In some of the foregoing embodiments of structure (X), n is an integer ranging from 2 to 6, for example, in some embodiments n is 2, 3, 4, 5 or 6. In some embodiments, n is 2. In some embodiments, n is 3. In some embodiments, n is 4.

In certain of the foregoing embodiments of structure (X), at least one of R¹, R², R³, R⁴ and R⁵ is unsubstituted. For example, in some embodiments, R¹, R², R³, R⁴ and R⁵ are each unsubstituted. In some embodiments, R³ is substituted. In other embodiments R⁴ is substituted. In still more embodiments, R⁵ is substituted. In certain specific embodiments, each of R³ and R⁴ are substituted. In some embodiments, a substituent on R³, R⁴ or R⁵ is hydroxyl. In certain embodiments, R³ and R⁴ are each substituted with hydroxyl.

In some of the foregoing embodiments of structure (X), at least one R is OH. In other embodiments, each R is H.

In various different embodiments of structure (X), the compound has one of the structures set forth in Table 9 below.

TABLE 9
Representative cationic lipids of structure (X)

| No. | Structure |
|---|---|
| X-1 | 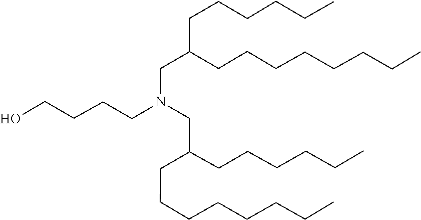 [SMILES: CCCCCCCCC(CCCCCC)CN(CCCCO)CC(CCCCCC)CCCCCCCC] |
| X-2 | 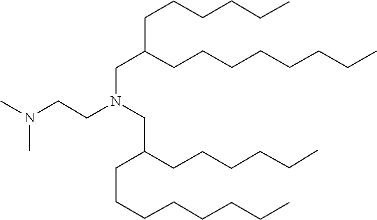 [SMILES: CCCCCCCCC(CCCCCC)CN(CCN(C)C)CC(CCCCCC)CCCCCCCC] |
| X-3 | 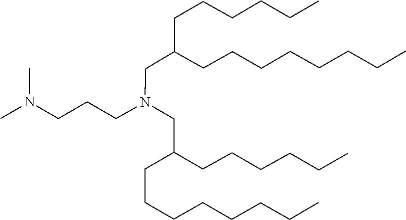 [SMILES: CCCCCCCCC(CCCCCC)CN(CCCN(C)C)CC(CCCCCC)CCCCCCCC] |
| X-4 | 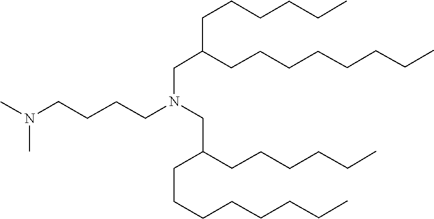 [SMILES: CCCCCCCCC(CCCCCC)CN(CCCCN(C)C)CC(CCCCCC)CCCCCCCC] |
| X-5 | 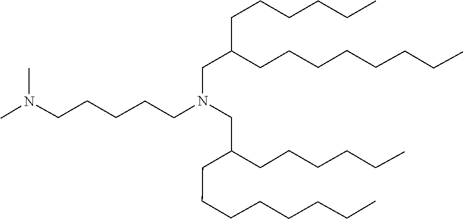 [SMILES: CCCCCCCCC(CCCCCC)CN(CCCCCN(C)C)CC(CCCCCC)CCCCCCCC] |
| X-6 | 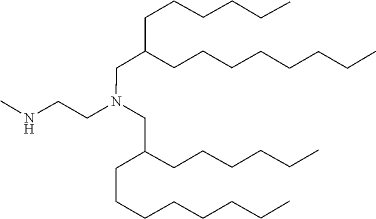 [SMILES: CCCCCCCCC(CCCCCC)CN(CCNC)CC(CCCCCC)CCCCCCCC] |
| X-7 | 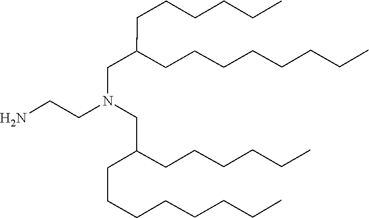 [SMILES: CCCCCCCCC(CCCCCC)CN(CCN)CC(CCCCCC)CCCCCCCC] |
| X-8 | 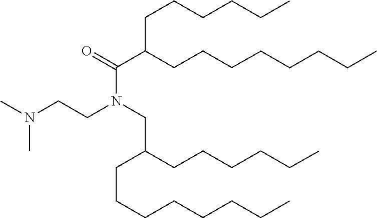 [SMILES: CCCCCCCCC(CCCCCC)CN(CCN(C)C)C(=O)C(CCCCCC)CCCCCCCC] |
| X-9 | 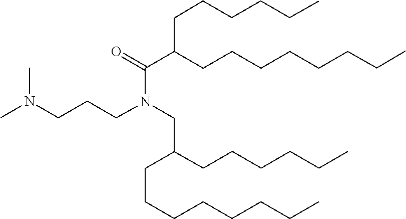 [SMILES: CCCCCCCCC(CCCCCC)CN(CCCN(C)C)C(=O)C(CCCCCC)CCCCCCCC] |
| X-10 | 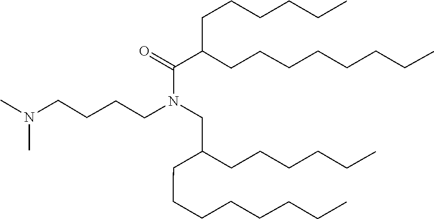 [SMILES: CCCCCCCCC(CCCCCC)CN(CCCCN(C)C)C(=O)C(CCCCCC)CCCCCCCC] |
| X-11 | 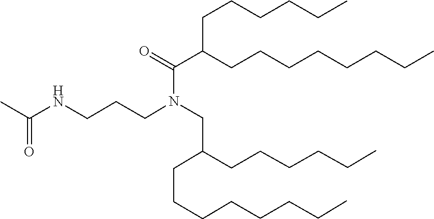 [SMILES: CCCCCCCCC(CCCCCC)CN(CCCNC(C)=O)C(=O)C(CCCCCC)CCCCCCCC] |
| X-12 | 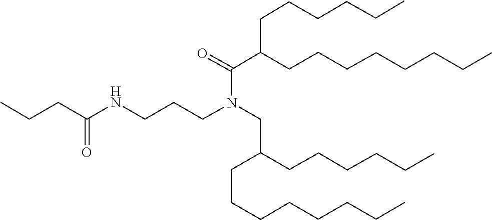 [SMILES: CCCCCCCCC(CCCCCC)CN(CCCNC(=O)CCC)C(=O)C(CCCCCC)CCCCCCCC] |
| X-13 | 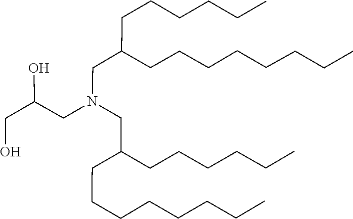 [SMILES: CCCCCCCCC(CCCCCC)CN(CC(O)CO)CC(CCCCCC)CCCCCCCC] |
| X-14 | 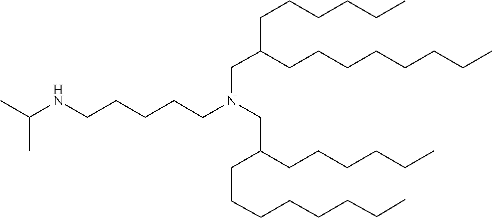 [SMILES: CCCCCCCCC(CCCCCC)CN(CCCCCNC(C)C)CC(CCCCCC)CCCCCCCC] |
| X-15 | 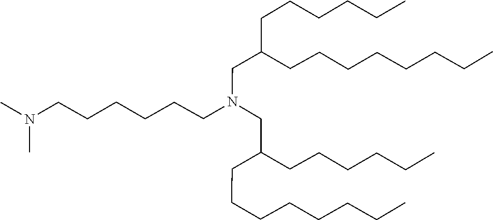 [SMILES: CCCCCCCCC(CCCCCC)CN(CCCCCCN(C)C)CC(CCCCCC)CCCCCCCC] |
| X-16 | 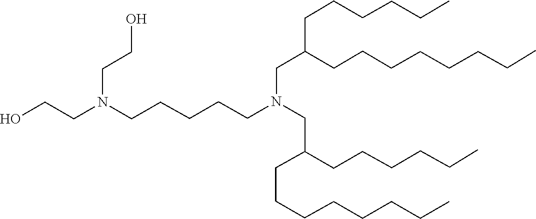 [SMILES: CCCCCCCCC(CCCCCC)CN(CCCCCN(CCO)CCO)CC(CCCCCC)CCCCCCCC] |
| X-17 | 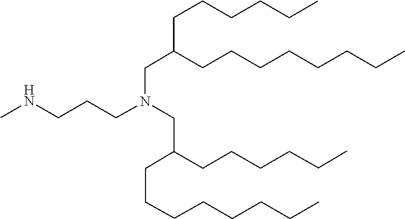 [SMILES: CCCCCCCCC(CCCCCC)CN(CCCNC)CC(CCCCCC)CCCCCCCC] |

In any of Embodiments 1, 2, 3, 4 or 5, the LNPs further comprise a neutral lipid. In various embodiments, the molar ratio of the cationic lipid to the neutral lipid ranges from about 2:1 to about 8:1. In certain embodiments, the neutral lipid is present in any of the foregoing LNPs in a concentration ranging from 5 to 10 mol percent, from 5 to 15 mol percent, 7 to 13 mol percent, or 9 to 11 mol percent. In certain specific embodiments, the neutral lipid is present in a concentration of about 9.5, 10 or 10.5 mol percent. In some embodiments, the molar ratio of cationic lipid to the neutral lipid ranges from about 4.1:1.0 to about 4.9:1.0, from about 4.5:1.0 to about 4.8:1.0, or from about 4.7:1.0 to 4.8:1.0. In some embodiments, the molar ratio of total cationic lipid to the neutral lipid ranges from about 4.1:1.0 to about 4.9:1.0, from about 4.5:1.0 to about 4.8:1.0, or from about 4.7:1.0 to 4.8:1.0.

Exemplary neutral lipids for use in any of Embodiments 1, 2, 3, 4 or 5 include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoylphosphatidyethanol amine (SOPE), and 1,2-dielaidoyl-sn-glycero-3-phophoethanolamine (transDOPE). In one embodiment, the neutral lipid is 1,2-distearoyl-sn-glycero-3phosphocholine (DSPC). In some embodiments, the neutral lipid is selected from DSPC, DPPC, DMPC, DOPC, POPC, DOPE and SM. In some embodiments, the neutral lipid is DSPC.

In various embodiments of Embodiments 1, 2, 3, 4 or 5, any of the disclosed lipid nanoparticles comprise a steroid or steroid analogue. In certain embodiments, the steroid or steroid analogue is cholesterol. In some embodiments, the steroid is present in a concentration ranging from 39 to 49 molar percent, 40 to 46 molar percent, from 40 to 44 molar percent, from 40 to 42 molar percent, from 42 to 44 molar percent, or from 44 to 46 molar percent. In certain specific embodiments, the steroid is present in a concentration of 40, 41, 42, 43, 44, 45, or 46 molar percent.

In certain embodiments, the molar ratio of cationic lipid to the steroid ranges from 1.0:0.9 to 1.0:1.2, or from 1.0:1.0 to 1.0:1.2. In some of these embodiments, the molar ratio of cationic lipid to cholesterol ranges from about 5:1 to 1:1. In certain embodiments, the steroid is present in a concentration ranging from 32 to 40 mol percent of the steroid.

In certain embodiments, the molar ratio of total cationic to the steroid ranges from 1.0:0.9 to 1.0:1.2, or from 1.0:1.0 to 1.0:1.2. In some of these embodiments, the molar ratio of total cationic lipid to cholesterol ranges from about 5:1 to 1:1. In certain embodiments, the steroid is present in a concentration ranging from 32 to 40 mol percent of the steroid.

In some embodiments of Embodiments 1, 2, 3 4 or 5, the LNPs further comprise a polymer conjugated lipid. In various other embodiments of Embodiments 1, 2, 3 4 or 5, the polymer conjugated lipid is a pegylated lipid. For example, some embodiments include a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate.

In various embodiments, the polymer conjugated lipid is present in a concentration ranging from 1.0 to 2.5 molar percent. In certain specific embodiments, the polymer conjugated lipid is present in a concentration of about 1.7 molar percent. In some embodiments, the polymer conjugated lipid is present in a concentration of about 1.5 molar percent.

In certain embodiments, the molar ratio of cationic lipid to the polymer conjugated lipid ranges from about 35:1 to about 25:1. In some embodiments, the molar ratio of cationic lipid to polymer conjugated lipid ranges from about 100:1 to about 20:1.

In certain embodiments, the molar ratio of total cationic lipid (i.e., the sum of the first and second cationic lipid) to the polymer conjugated lipid ranges from about 35:1 to about 25:1. In some embodiments, the molar ratio of total cationic lipid to polymer conjugated lipid ranges from about 100:1 to about 20:1.

In some embodiments of Embodiments 1, 2, 3 4 or 5, the pegylated lipid, when present, has the following Formula (XI):

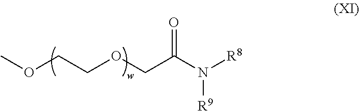

or a pharmaceutically acceptable salt, tautomer or stereoisomer thereof, wherein:

R¹² and R¹³ are each independently a straight or branched, saturated or unsaturated alkyl chain containing from 10 to 30 carbon atoms, wherein the alkyl chain is optionally interrupted by one or more ester bonds; and

w has a mean value ranging from 30 to 60.

In some embodiments, R¹² and R¹³ are each independently straight, saturated alkyl chains containing from 12 to 16 carbon atoms. In other embodiments, the average w ranges from 42 to 55, for example, the average w is 42, 43, 44, 45, 46, 47, 48, 49, 50, 51, 52, 53, 54 or 55. In some specific embodiments, the average w is about 49.

In some embodiments, the pegylated lipid has the following Formula (XIa):

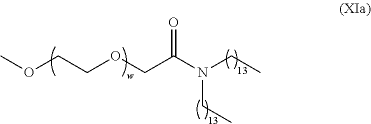

wherein the average w is about 49.

In some embodiments of Embodiments 1, 2, 3 4 or 5, the nucleic acid is selected from antisense and messenger RNA. For example, messenger RNA may be used to induce an immune response (e.g., as a vaccine), for example by translation of immunogenic proteins.

In other embodiments of Embodiments 1, 2, 3 4 or 5, the nucleic acid is mRNA, and the mRNA to lipid ratio in the LNP (i.e., N/P, were N represents the moles of cationic lipid and P represents the moles of phosphate present as part of the nucleic

In an embodiment, the transfer vehicle comprises a lipid or an ionizable lipid described in US patent publication number 20190314524.

Some embodiments of the present invention provide nucleic acid-lipid nanoparticle compositions comprising one or more of the novel cationic lipids described herein as structures listed in Table 10, that provide increased activity of the nucleic acid and improved tolerability of the compositions in vivo.

In one embodiment, an ionizable lipid has the following structure (XII):

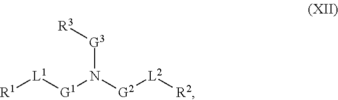

or a pharmaceutically acceptable salt, tautomer, prodrug or stereoisomer thereof, wherein:

one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, C(═O)NR^{a}—, NR^{a}C(═O)NR^{a}—, —OC(═O)NR^{a}— or —NR^{a}C(═O)O— or a direct bond;

G¹ and G² are each independently unsubstituted C₁-C₁₂ alkylene or C₁-C₁₂ alkenylene;

G³ is C₁-C₂₄ alkylene, C₁-C₂₄ alkenylene, C₃-C₈ cycloalkylene, C₃-C₈ cycloalkenylene;

R^{a} is H or C₁-C₁₂ alkyl;

R¹ and R² are each independently C₆-C₂₄ alkyl or C₆-C₂₄ alkenyl;

R³ is H, OR⁵, CN, —C(═O)OR⁴, —OC(═O)R⁴ or —NR⁵C(═O)R⁴;

R⁴ is C₁-C₁₂ alkyl;

R⁵ is H or C₁-C₆ alkyl; and

x is 0, 1 or 2.

In some embodiments, an ionizable lipid has one of the following structures (XIIA) or (XIIB):

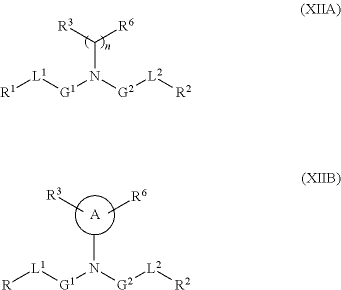

wherein:

A is a 3 to 8-membered cycloalkyl or cycloalkylene ring;

R⁶ is, at each occurrence, independently H, OH or C₁-C₂₄ alkyl; and

n is an integer ranging from 1 to 15.

In some embodiments, the ionizable lipid has structure (XIIA), and in other embodiments, the ionizable lipid has structure (XIIB)

In other embodiments, an ionizable lipid has one of the following structures (XIIC) or (XIID):

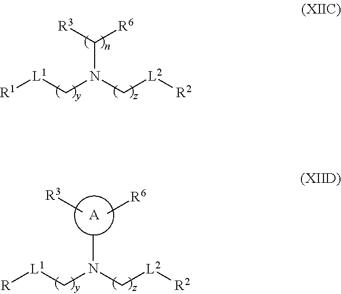

wherein y and z are each independently integers ranging from 1 to 12.

In some embodiments, one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some embodiments, an ionizable lipid has one of the following structures (XIIE) or (XIIF):

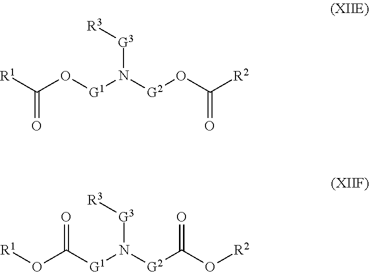

In some embodiments, an ionizable lipid has one of the following structures (XIIG), (XIIH), (XIII), or (XIIJ):

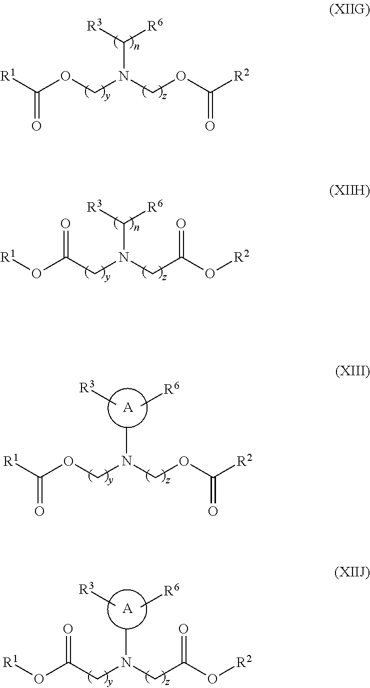

In some embodiments, n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some embodiments, y and z are each independently an integer ranging from 2 to 10. For example, in some embodiments, y and z are each independently an integer ranging from 4 to 9 or from 4 to 6.

In some embodiments, R⁶ is H. In other embodiments, R⁶ is C₁-C₂₄ alkyl. In other embodiments, R⁶ is OH.

In some embodiments, G³ is unsubstituted. In other embodiments, G3 is substituted. In various different embodiments, G³ is linear C₁-C₂₄ alkylene or linear C₁-C₂₄ alkenylene.

In some embodiments, R¹ or R², or both, is C₆-C₂₄ alkenyl. For example, in some embodiments, R¹ and R² each, independently have the following structure:

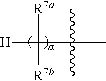

wherein:

R^{7a} and R^{7b} are, at each occurrence, independently H or C₁-C₁₂ alkyl; and

a is an integer from 2 to 12,

wherein R^{7a}, R^{7b} and a are each selected such that R¹ and R² each independently comprise from 6 to 20 carbon atoms.

In some embodiments, a is an integer ranging from 5 to 9 or from 8 to 12.

In some embodiments, at least one occurrence of R^{7a} is H. For example, in some embodiments, R^{7a} is H at each occurrence. In other different embodiments, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments, R¹ or R², or both, has one of the following structures:

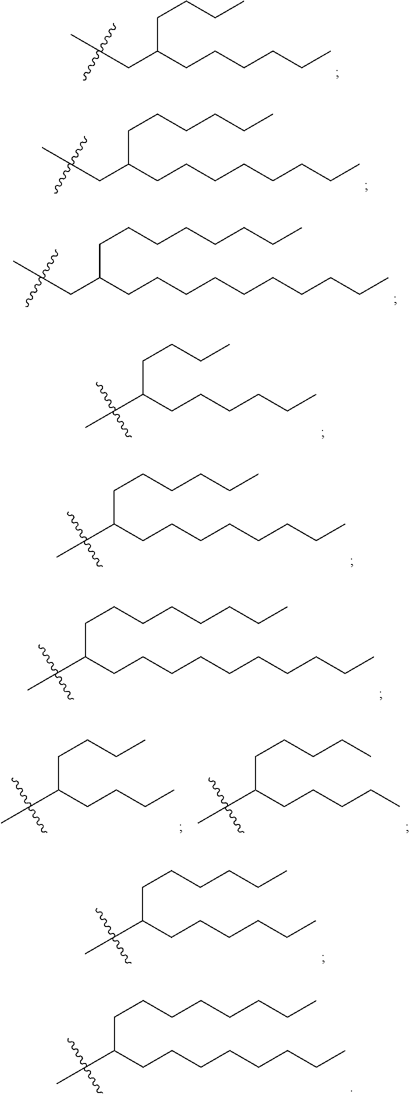

In some embodiments, R³ is —OH, —CN, —C(═O)OR⁴, —OC(═O)R⁴ or NHC(═O)R⁴. In some embodiments, R⁴ is methyl or ethyl.

In some embodiments, an ionizable lipid is a compound of Formula (1):

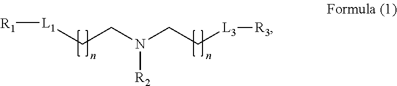

wherein:

each n is independently 1, 2, 3, 4, 5, 6, 7, 8, 9, 10, 11, 12, 13, 14, or 15; and

L₁ and L₃ are each independently —OC(O)—* or —C(O)O—*, wherein “*” indicates the attachment point to R₁ or R₃;

R₁ and R₃ are each independently a linear or branched C₉-C₂₀ alkyl or C₉-C₂₀ alkenyl, optionally substituted by one or more substituents selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkyl sulfoxidealkyl, alkylsulfonyl, and alkylsulfonealkyl.

In some embodiments, R₁ and R₃ are the same. In some embodiments, R₁ and R₃ are different.

In some embodiments, R₁ and R₃ are each independently a branched saturated C₉-C₂₀ alkyl. In some embodiments, one of R₁ and R₃ is a branched saturated C₉-C₂₀ alkyl, and the other is an unbranched saturated C₉-C₂₀ alkyl. In some embodiments, R₁ and R₃ are each independently selected from a group consisting of:

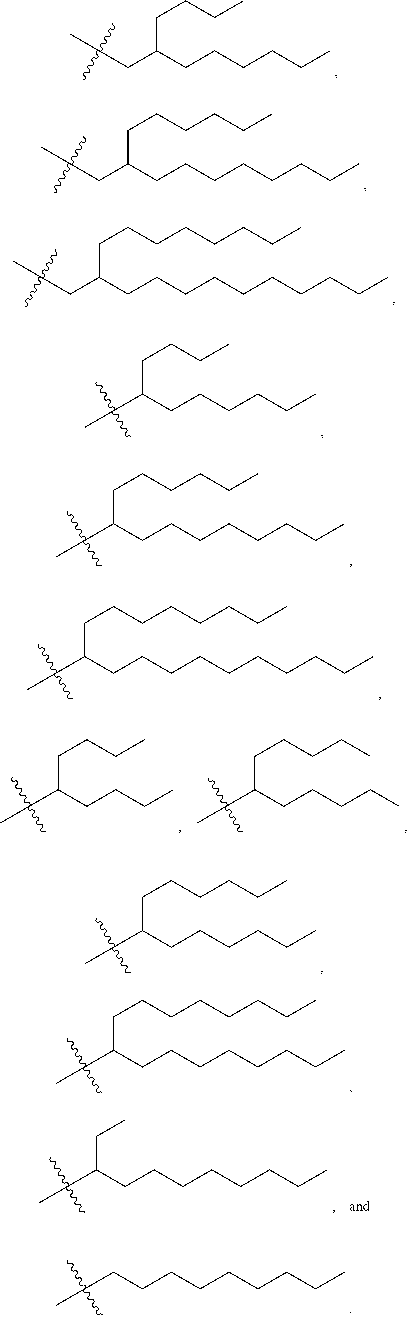

In various embodiments, R₂ is selected from a group consisting of:

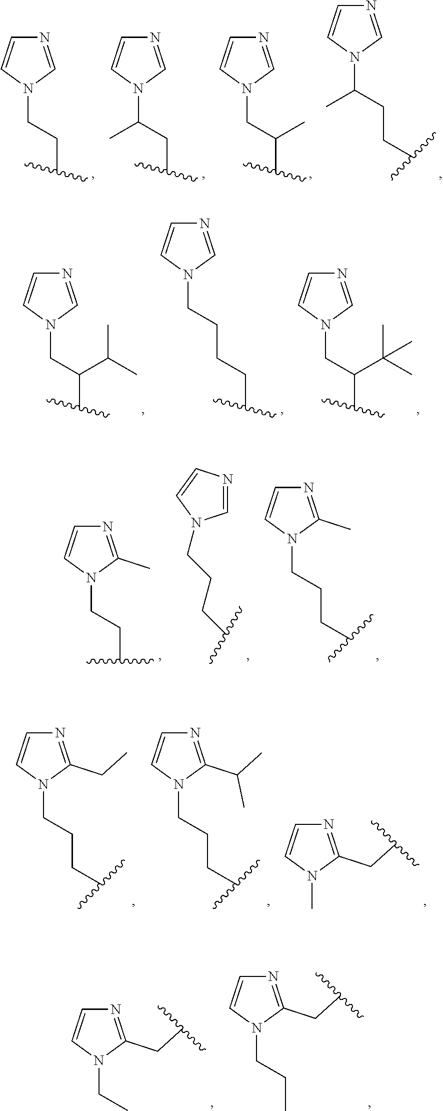

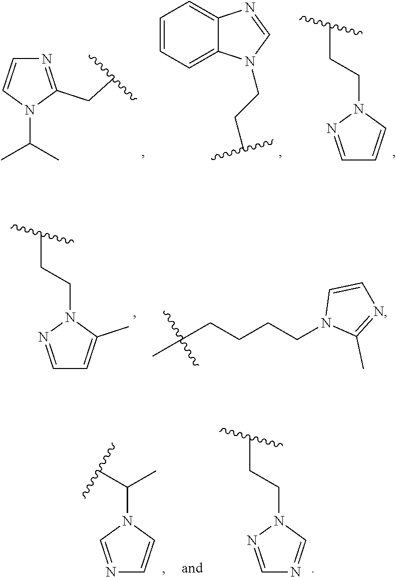

In some embodiments, R₂ may be as described in International Pat. Pub. No. WO2019/152848 A1, which is incorporated herein by reference in its entirety.

In some embodiments, an ionizable lipid is a compound of Formula (1-1) or Formula (1-2):

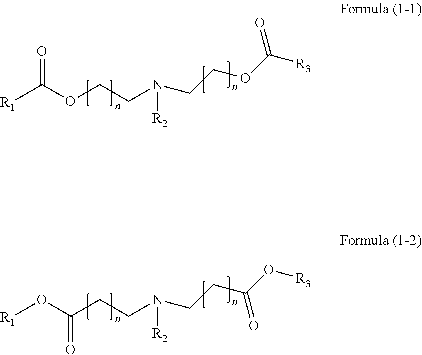

wherein n, R₁, R₂, and R₃ are as defined in Formula (1).

Preparation methods for the above compounds and compositions are described herein below and/or known in the art.

It will be appreciated by those skilled in the art that in the process described herein the functional groups of intermediate compounds may need to be protected by suitable protecting groups. Such functional groups include, e.g., hydroxyl, amino, mercapto, and carboxylic acid. Suitable protecting groups for hydroxyl include, e.g., trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino, and guanidino include, e.g., t-butoxycarbonyl, benzyloxycarbonyl, and the like. Suitable protecting groups for mercapto include, e.g., —C(O)—R″ (where R″ is alkyl, aryl, or arylalkyl), p-methoxybenzyl, trityl, and the like. Suitable protecting groups for carboxylic acid include, e.g., alkyl, aryl, or arylalkyl esters. Protecting groups may be added or removed in accordance with standard techniques, which are known to one skilled in the art and as described herein. The use of protecting groups is described in detail in, e.g., Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin, or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of the invention which are pharmacologically active. Such derivatives may therefore be described as prodrugs. All prodrugs of compounds of this invention are included within the scope of the invention.

Furthermore, all compounds of the invention which exist in free base or acid form can be converted to their pharmaceutically acceptable salts by treatment with the appropriate inorganic or organic base or acid by methods known to one skilled in the art. Salts of the compounds of the invention can also be converted to their free base or acid form by standard techniques.

The following reaction scheme illustrates an exemplary method to make compounds of Formula (1):

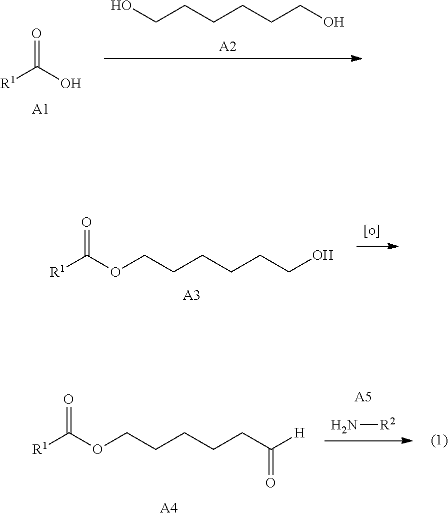

A1 are purchased or prepared according to methods known in the art. Reaction of A1 with diol A2 under appropriate condensation conditions (e.g., DCC) yields ester/alcohol A3, which can then be oxidized (e.g., with PCC) to aldehyde A4. Reaction of A4 with amine A5 under reductive amination conditions yields a compound of Formula (1).

The following reaction scheme illustrates a second exemplary method to make compounds of Formula (1), wherein R₁ and R₃ are the same:

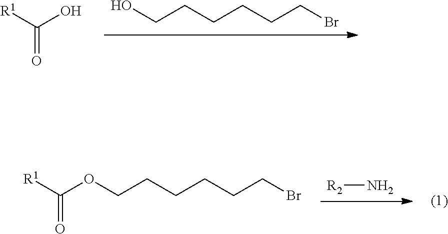

Modifications to the above reaction scheme, such as using protecting groups, may yield compounds wherein R₁ and R₃ are different. The use of protecting groups, as well as other modification methods, to the above reaction scheme will be readily apparent to one of ordinary skill in the art.

It is understood that one skilled in the art may be able to make these compounds by similar methods or by combining other methods known to one skilled in the art. It is also understood that one skilled in the art would be able to make other compounds of Formula (1) not specifically illustrated herein by using the appropriate starting materials and modifying the parameters of the synthesis. In general, starting materials may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Advanced Organic Chemistry: Reactions, Mechanisms, and Structure, 5th edition (Wiley, December 2000)) or prepared as described in this invention.

In some embodiments, an ionizable lipid is a compound of Formula (2):

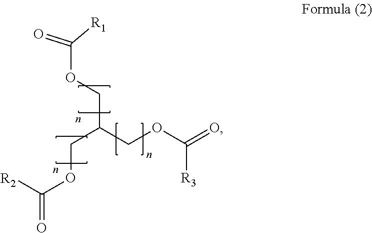

wherein each n is independently 1, 2, 3, 4, 5, 6, 7, 8, 9, 10, 11, 12, 13, 14, or 15.

In some embodiments, as used in Formula (2), R₁ and R₂ are as defined in Formula (1).

In some embodiments, as used in Formula (2), R₁ and R₂ are each independently selected from a group consisting of:

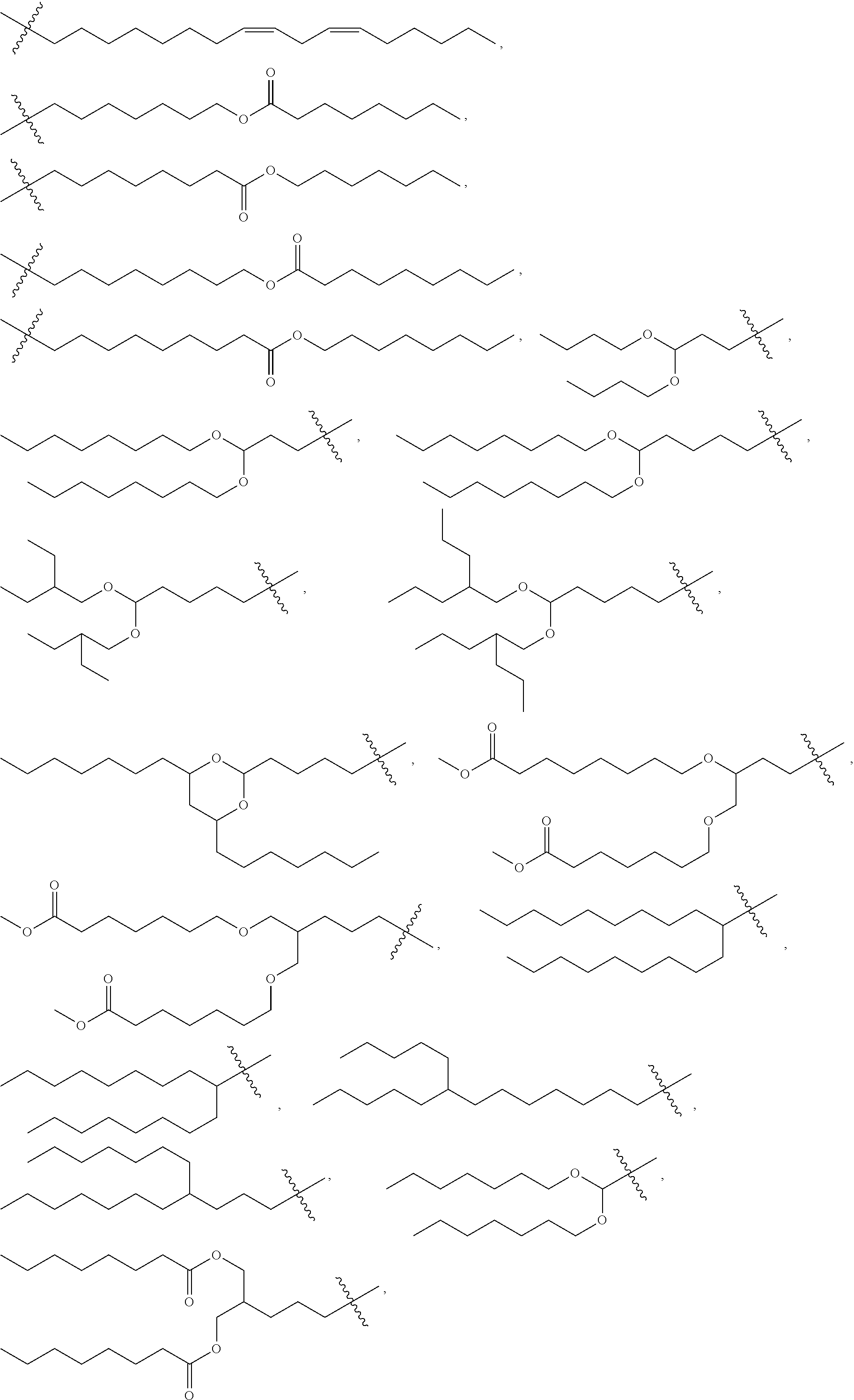

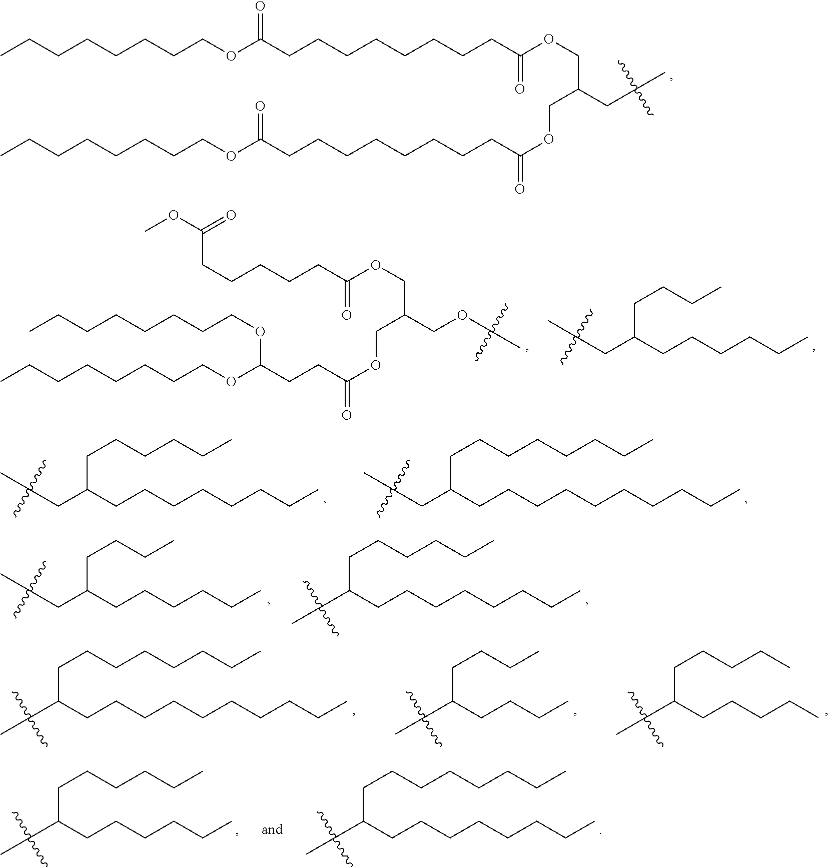

In some embodiments, R₁ and/or R₂ as used in Formula (2) may be as described in International Pat. Pub. No. WO2015/095340 A1, which is incorporated herein by reference in its entirety. In some embodiments, R₁ as used in Formula (2) may be as described in International Pat. Pub. No. WO2019/152557 A1, which is incorporated herein by reference in its entirety.

In some embodiments, as used in Formula (2), R₃ is selected from a group consisting of:

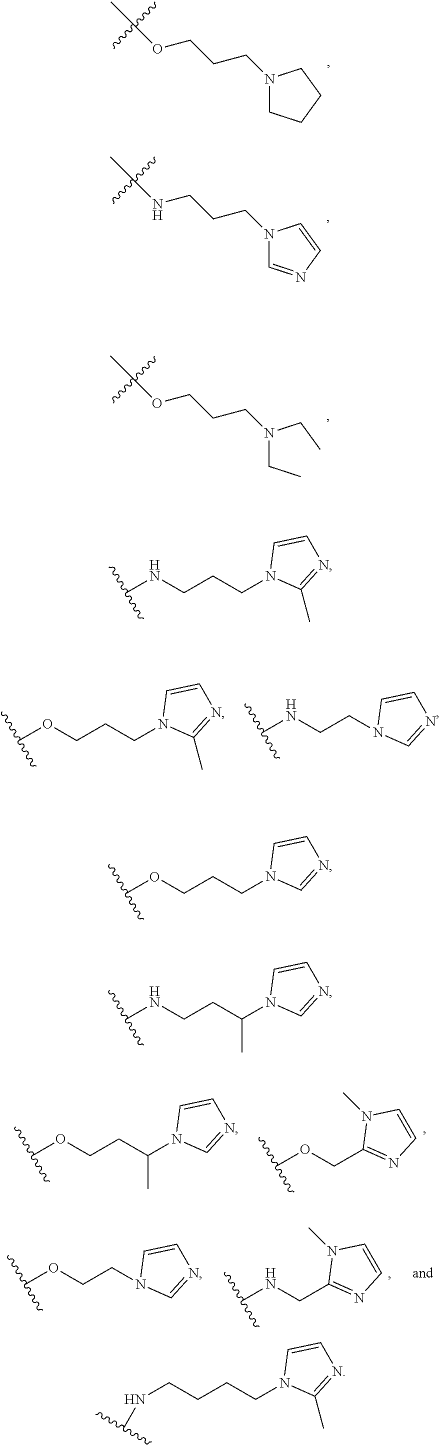

In some embodiments, an ionizable lipid is a compound of Formula (3)

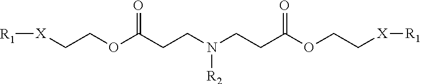

wherein X is selected from —O—, —S—, or —OC(O)—*, wherein * indicates the attachment point to R₁.

In some embodiments, an ionizable lipid is a compound of Formula (3-1):

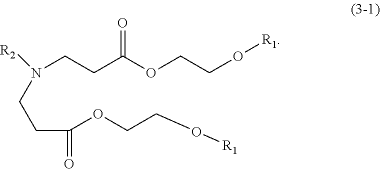

In some embodiments, an ionizable lipid is a compound of Formula (3-2):

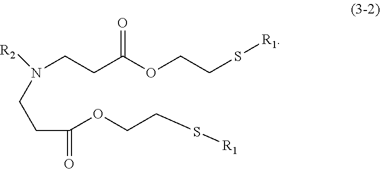

In some embodiments, an ionizable lipid is a compound of Formula (3-3):

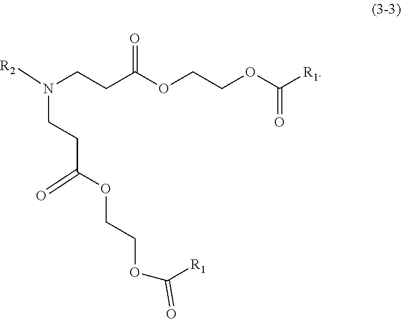

In some embodiments, as used in Formula (3-1), (3-2), or (3-3), each R₁ is independently a branched saturated C₉-C₂₀ alkyl. In some embodiments, each R₁ is independently selected from a group consisting of:

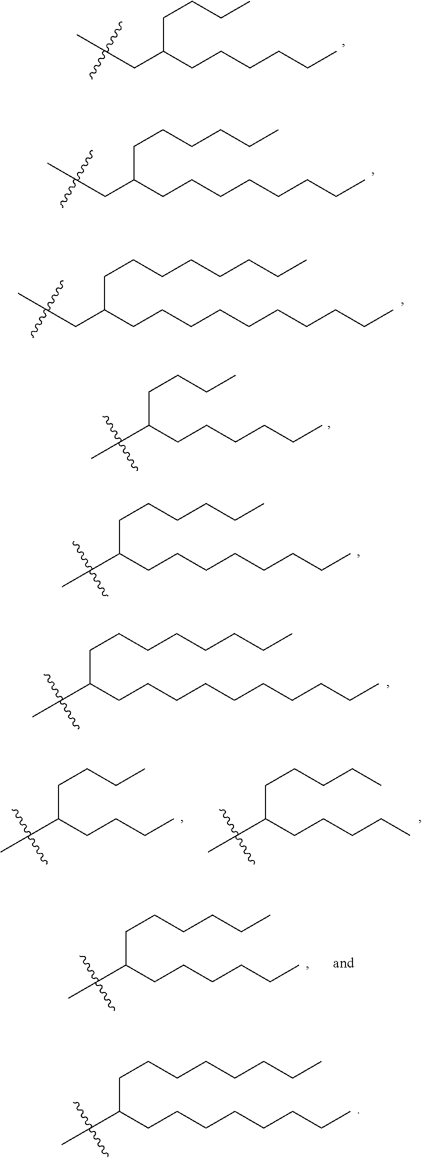

In some embodiments, each R₁ in Formula (3-1), (3-2), or (3-3) are the same.

In some embodiments, as used in Formula (3-1), (3-2), or (3-3), R₂ is selected from a group consisting of:

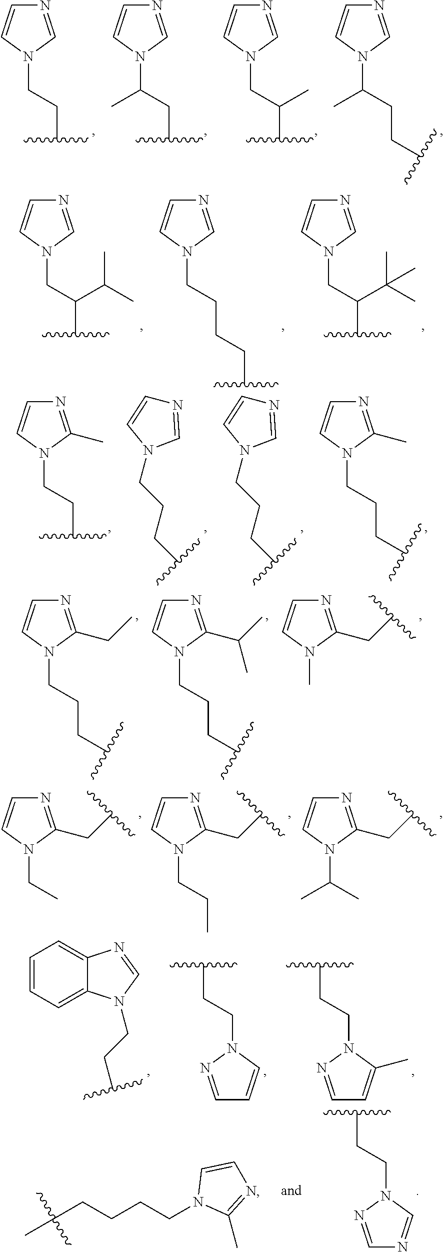

In some embodiments, R₂ as used in Formula (3-1), (3-2), or (3-3) may be as described in International Pat. Pub. No. WO2019/152848A1, which is incorporated herein by reference in its entirety.

In some embodiments, an ionizable lipid is a compound of Formula (5):

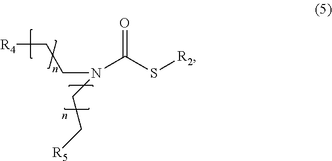

wherein:

each n is independently 1, 2, 3, 4, 5, 6, 7, 8, 9, 10, 11, 12, 13, 14, or 15; and

R₂ is as defined in Formula (1).

In some embodiments, as used in Formula (5), R₄ and R₅ are defined as R₁ and R₃, respectively, in Formula (1). In some embodiments, as used in Formula (5), R₄ and R₅ may be as described in International Pat. Pub. No. WO2019/191780 A1, which is incorporated herein by reference in its entirety.

In some embodiments, an ionizable lipid is a compound of Formula (6):

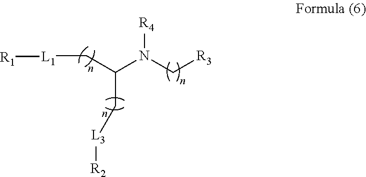

wherein:

each n is independently an integer from 0-15;

L₁ and L₃ are each independently —OC(O)—* or —C(O)O—*, wherein “*” indicates the attachment point to R₁ or R₃;

R₁ and R₂ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkyl sulfoxidealkyl, alkylsulfonyl, and alkylsulfonealkyl;

R₃ is selected from a group consisting of:

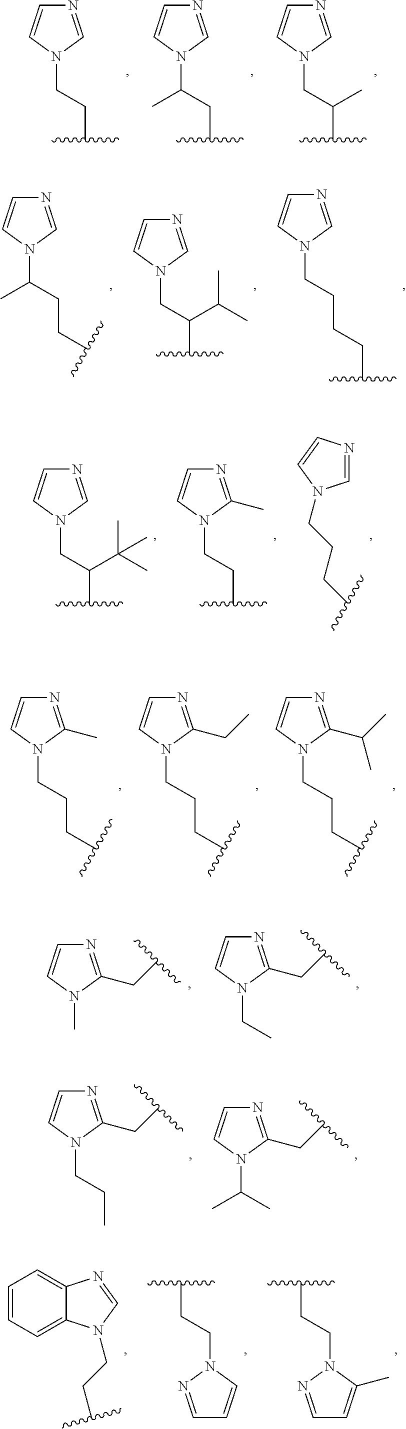

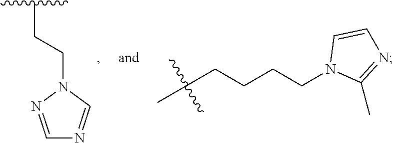 [SMILES: *CCn1cncn1 ; *CCCCn1ccnc1C]

and

R₄ is a linear or branched C₁-C₁₅ alkyl or C₁-C₁₅ alkenyl.

In some embodiments, R₁ and R₂ are each independently selected from a group consisting of:

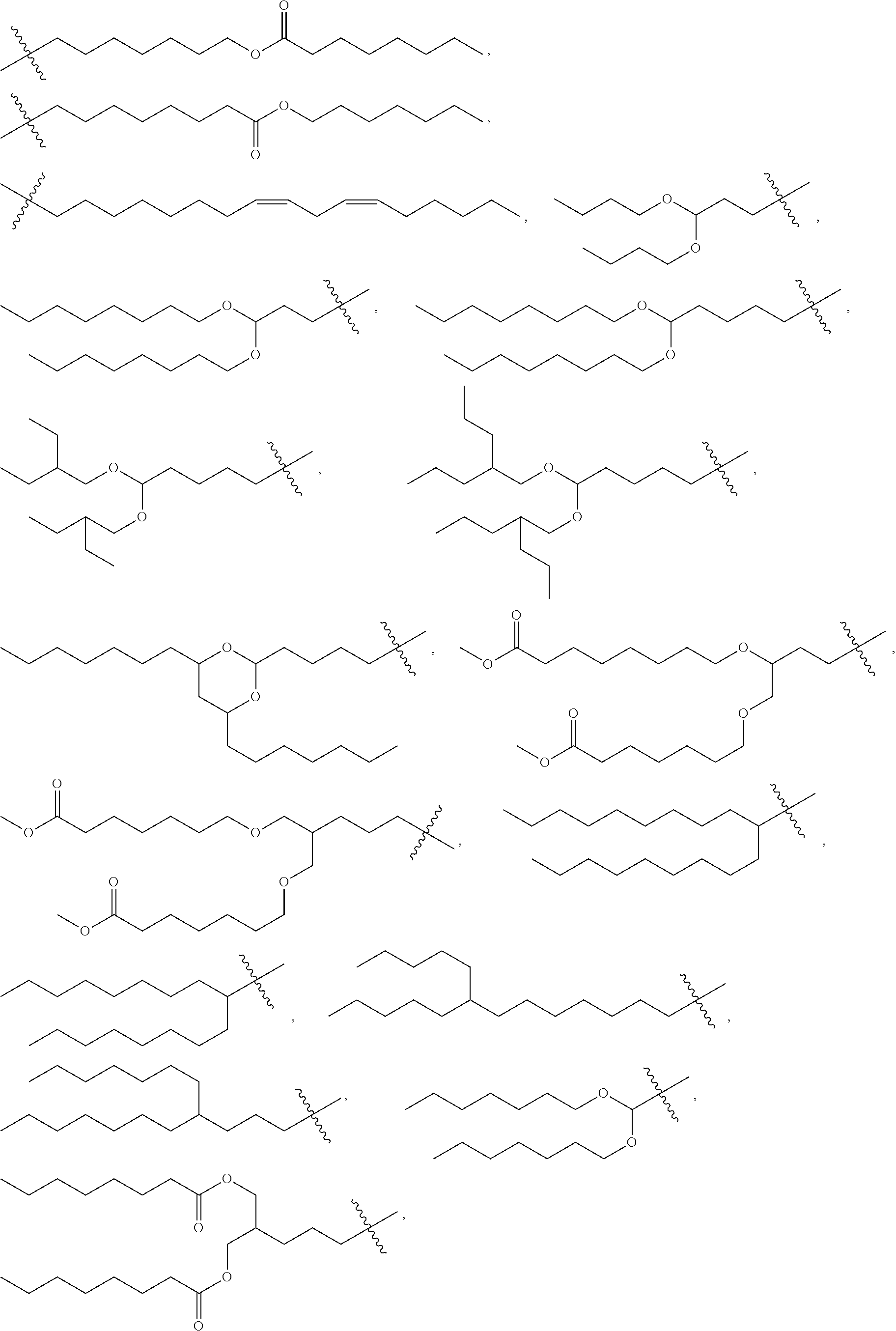

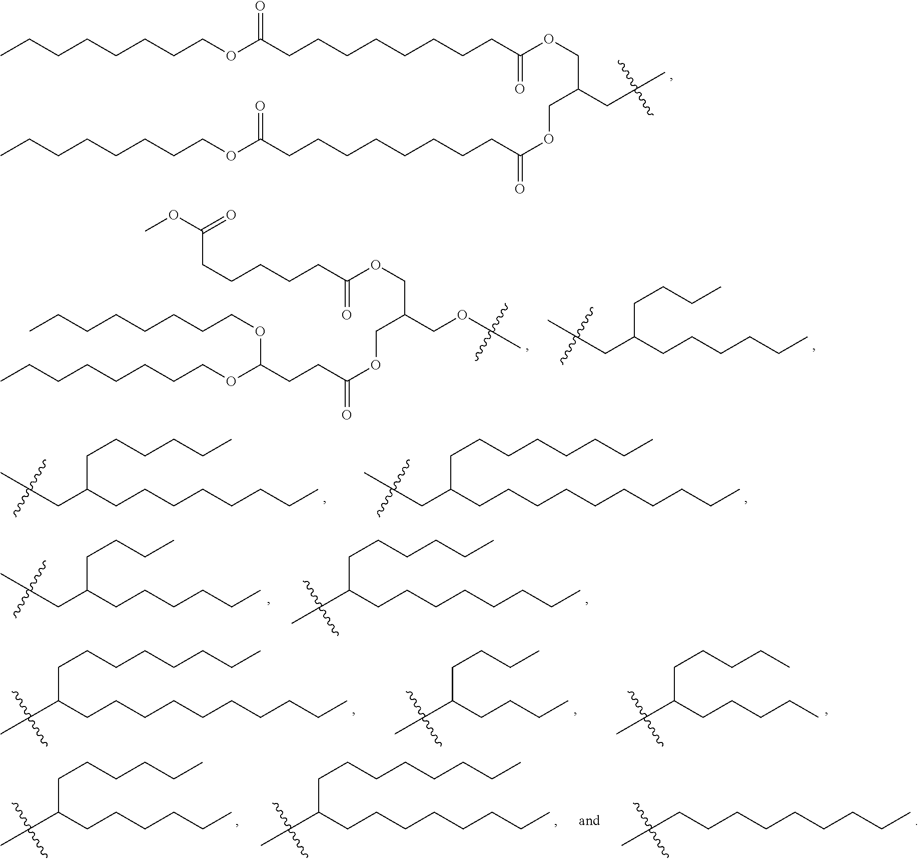

In some embodiments, R₁ and R₂ are the same. In some embodiments, R₁ and R₂ are different.

In some embodiments, an ionizable lipid of the disclosure is selected from Table 10a. In some embodiments, the ionizable lipid is Lipid 26 in Table 10a. In some embodiments, the ionizable lipid is Lipid 27 in Table 10a. In some embodiments, the ionizable lipid is Lipid 53 in Table 10a. In some embodiments, the ionizable lipid is Lipid 54 in Table 10a. In some embodiments, the ionizable lipid is Lipid 45 in Table 10a. In some embodiments, the ionizable lipid is Lipid 46 in Table 10a. In some embodiments, the ionizable lipid is Lipid 137 in Table 10a. In some embodiments, the ionizable lipid is Lipid 138 in Table 10a. In some embodiments, the ionizable lipid is Lipid 139 in Table 10a. In some embodiments, the ionizable lipid is Lipid 128 in Table 10a. In some embodiments, the ionizable lipid is Lipid 130 in Table 10a.

In some embodiments, an ionizable lipid of the disclosure is selected from the group consisting of:

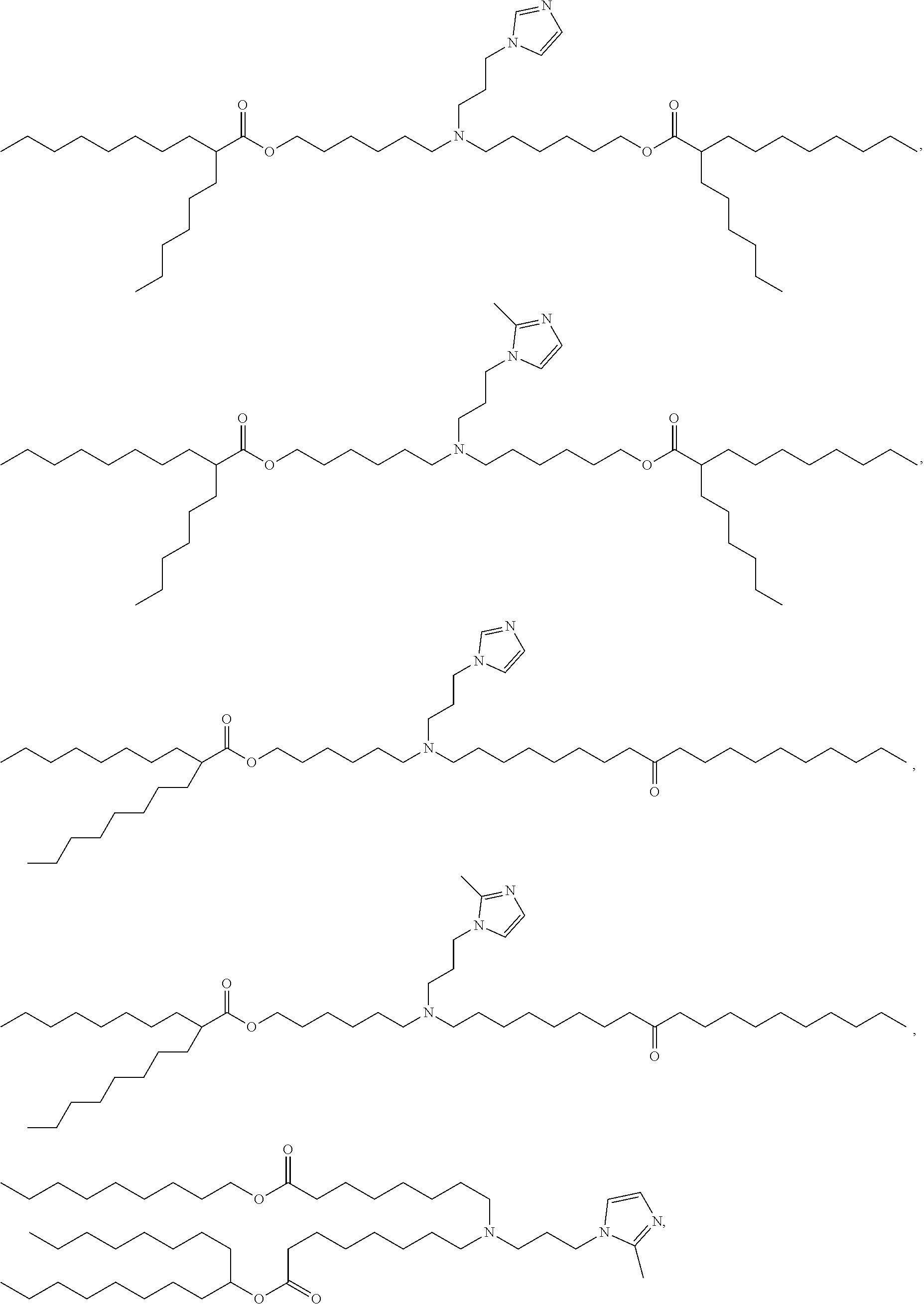

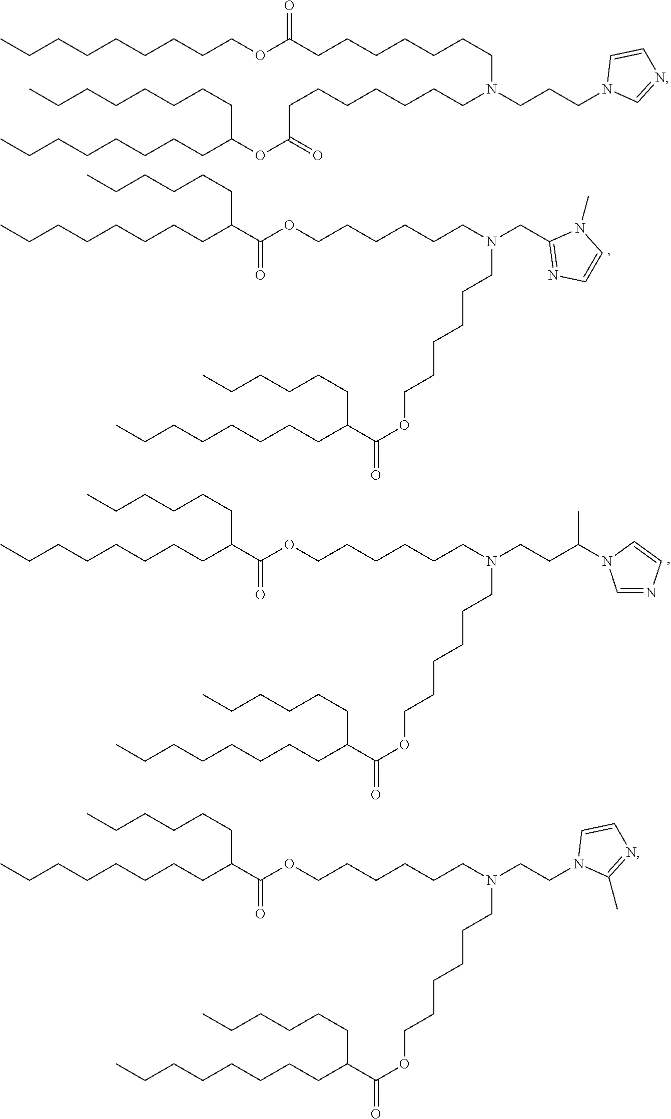

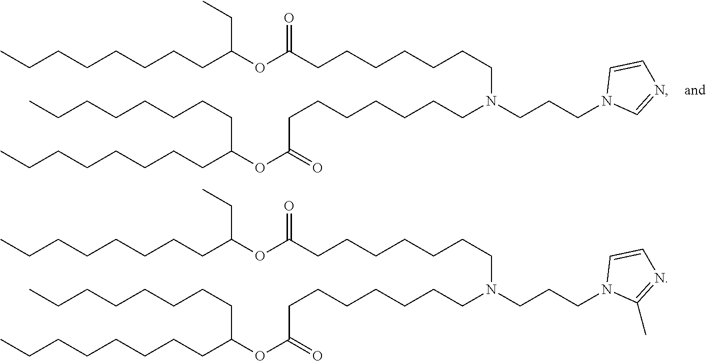

In some embodiments, an ionizable lipid of the disclosure is selected from the group consisting of:

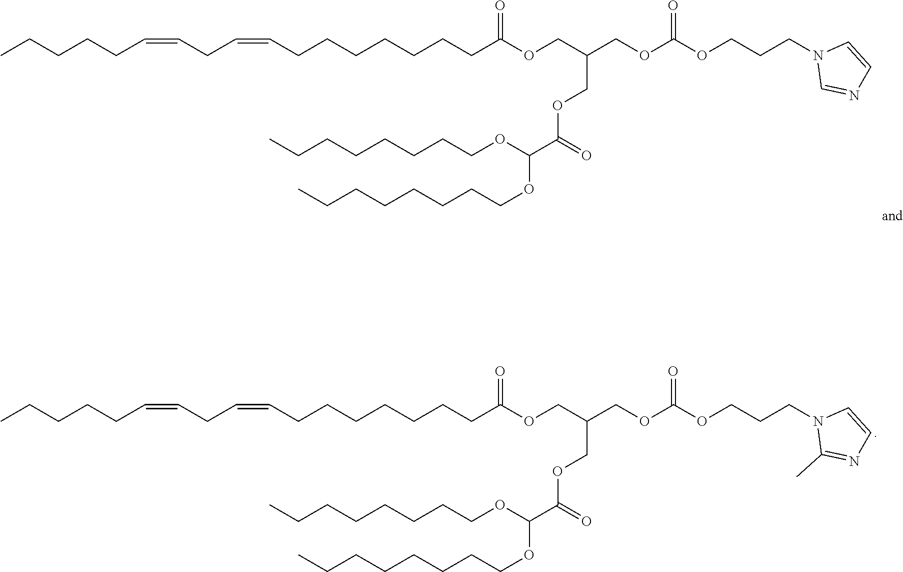 [SMILES: CCCCC/C=C\C/C=C\CCCCCCCC(=O)OCC(COC(=O)OCCCn1ccnc1)COC(=O)C(OCCCCCCCC)OCCCCCCCC ; CCCCC/C=C\C/C=C\CCCCCCCC(=O)OCC(COC(=O)OCCCn1ccnc1C)COC(=O)C(OCCCCCCCC)OCCCCCCCC]

In some embodiments, an ionizable lipid of the disclosure is selected from the group consisting of:

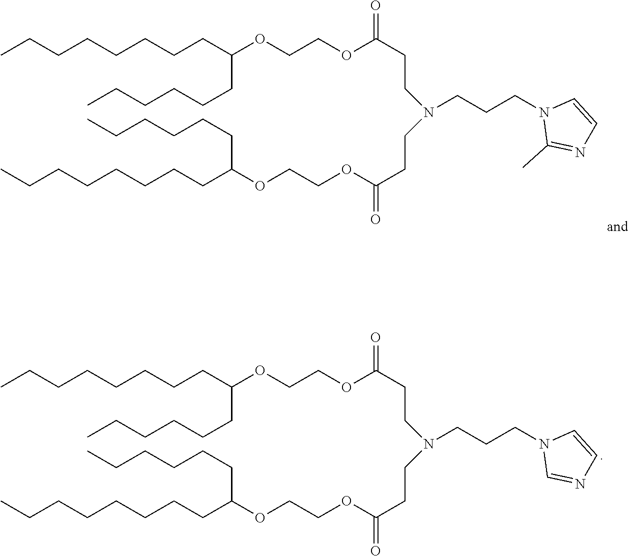 [SMILES: CCCCCCCCC(CCCCCC)OCCOC(=O)CCN(CCCn1ccnc1C)CCC(=O)OCCOC(CCCCCC)CCCCCCCC ; CCCCCCCCC(CCCCCC)OCCOC(=O)CCN(CCCn1ccnc1)CCC(=O)OCCOC(CCCCCC)CCCCCCCC]

In some embodiments, an ionizable lipid of the disclosure is selected from the group consisting of:

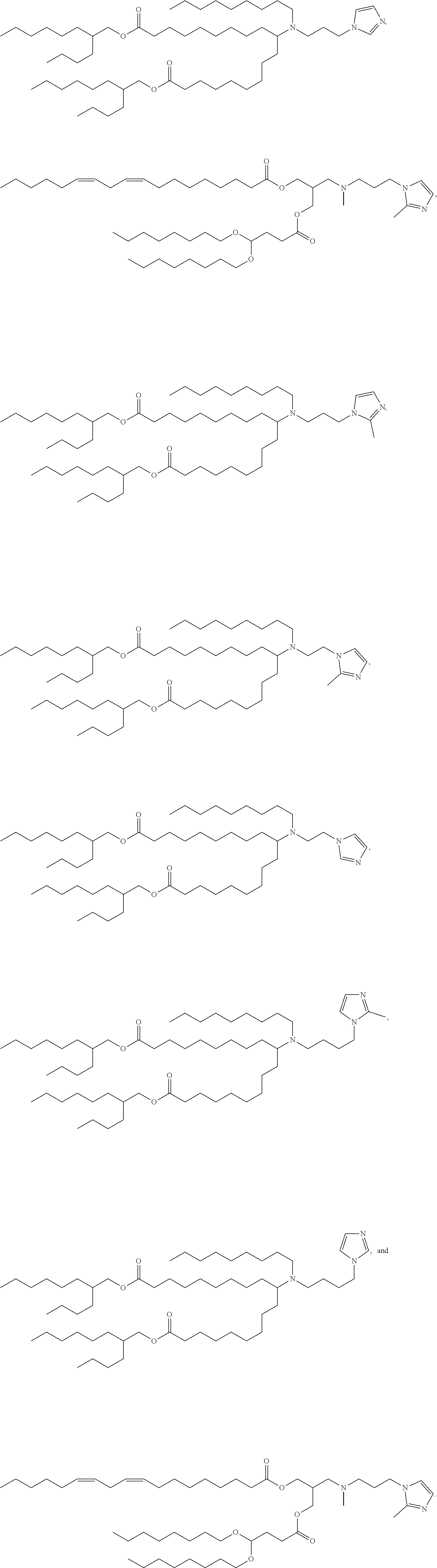

TABLE 10a

| Ionizable lipid | |
|---|---|
| number | Structure |
| 1 | 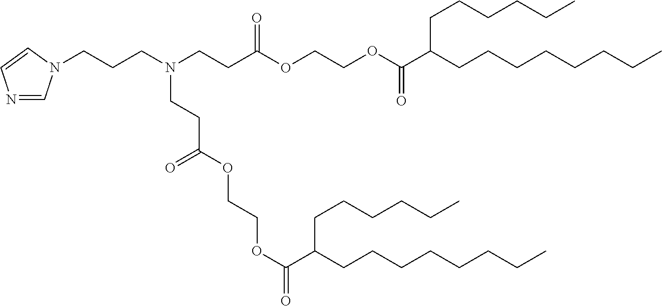 [SMILES: CCCCCCCCC(CCCCCC)C(=O)OCCOC(=O)CCN(CCCn1ccnc1)CCC(=O)OCCOC(=O)C(CCCCCC)CCCCCCCC] |
| 2 | 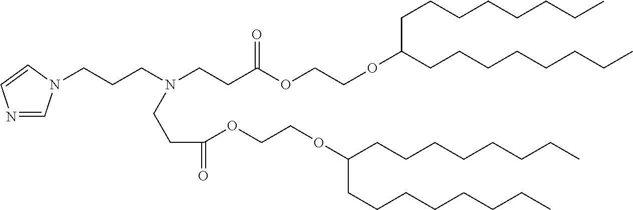 [SMILES: CCCCCCCCC(CCCCCCCC)OCCOC(=O)CCN(CCCn1ccnc1)CCC(=O)OCCOC(CCCCCCCC)CCCCCCCC] |
| 3 | 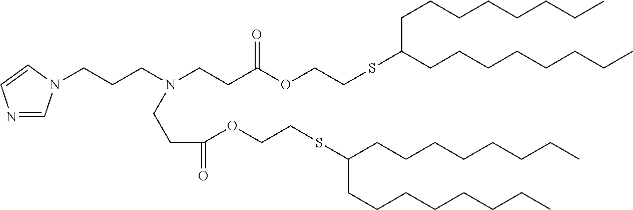 [SMILES: CCCCCCCCC(CCCCCCCC)SCCOC(=O)CCN(CCCn1ccnc1)CCC(=O)OCCSC(CCCCCCCC)CCCCCCCC] |
| 4 | 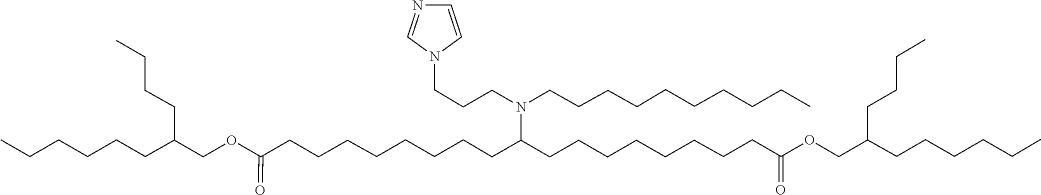 [SMILES: CCCCCCCCCCN(CCCn1ccnc1)C(CCCCCCCCC(=O)OCC(CCCC)CCCCCC)CCCCCCCCC(=O)OCC(CCCC)CCCCCC] |
| 5 | 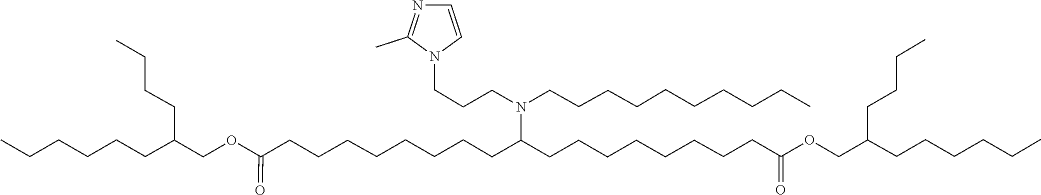 [SMILES: CCCCCCCCCCN(CCCn1ccnc1C)C(CCCCCCCCC(=O)OCC(CCCC)CCCCCC)CCCCCCCCC(=O)OCC(CCCC)CCCCCC] |
| 6 | 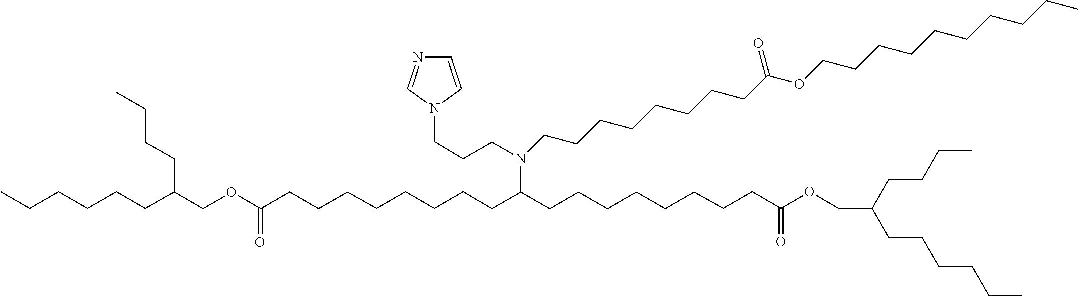 [SMILES: CCCCCCCCCCOC(=O)CCCCCCCCN(CCCn1ccnc1)C(CCCCCCCCC(=O)OCC(CCCC)CCCCCC)CCCCCCCCC(=O)OCC(CCCC)CCCCCC] |
| 7 | 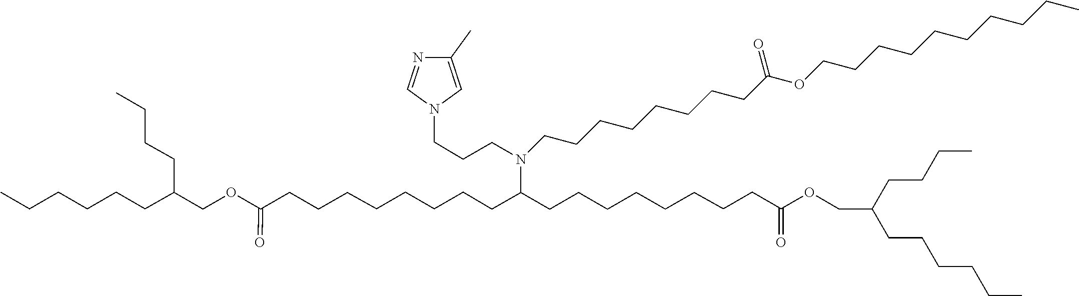 [SMILES: CCCCCCCCCCOC(=O)CCCCCCCCN(CCCn1cnc(C)c1)C(CCCCCCCCC(=O)OCC(CCCC)CCCCCC)CCCCCCCCC(=O)OCC(CCCC)CCCCCC] |
| 8 | 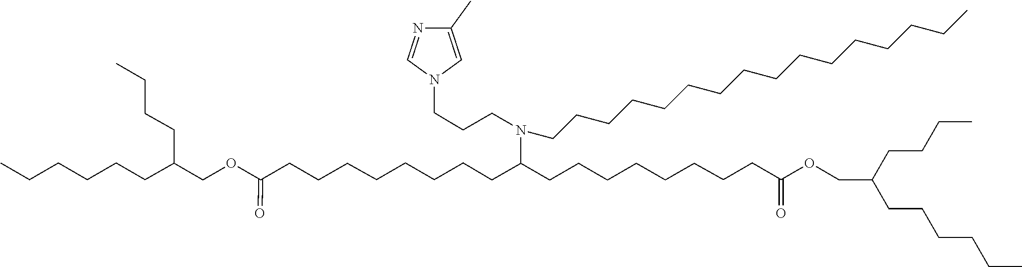 [SMILES: CCCCCCCCCCCCCCCCN(CCCn1cnc(C)c1)C(CCCCCCCCC(=O)OCC(CCCC)CCCCCC)CCCCCCCCC(=O)OCC(CCCC)CCCCCC] |
| 9 | 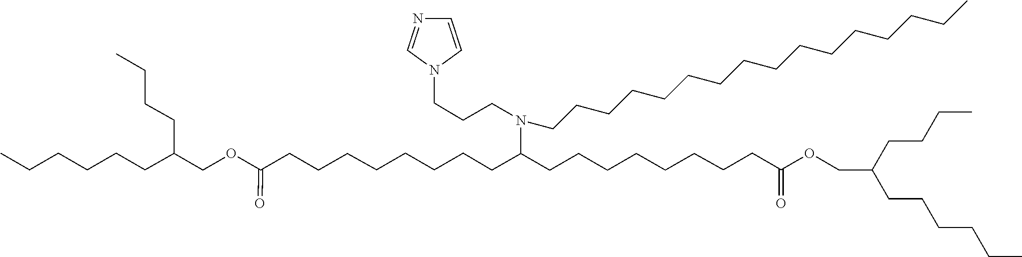 [SMILES: CCCCCCCCCCCCCCCCN(CCCn1ccnc1)C(CCCCCCCCC(=O)OCC(CCCC)CCCCCC)CCCCCCCCC(=O)OCC(CCCC)CCCCCC] |
| 10 | 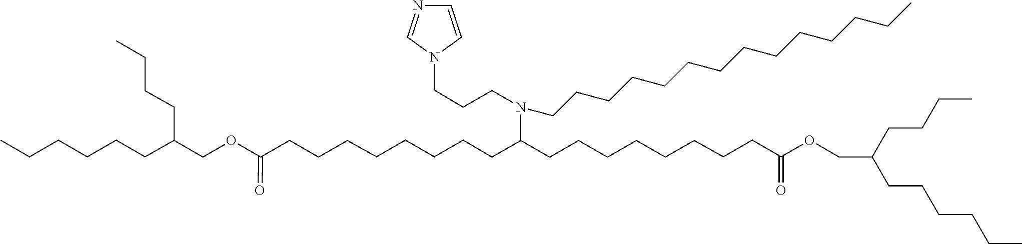 [SMILES: CCCCCCCCCCCCCCN(CCCn1ccnc1)C(CCCCCCCCC(=O)OCC(CCCC)CCCCCC)CCCCCCCCC(=O)OCC(CCCC)CCCCCC] |
| 11 | 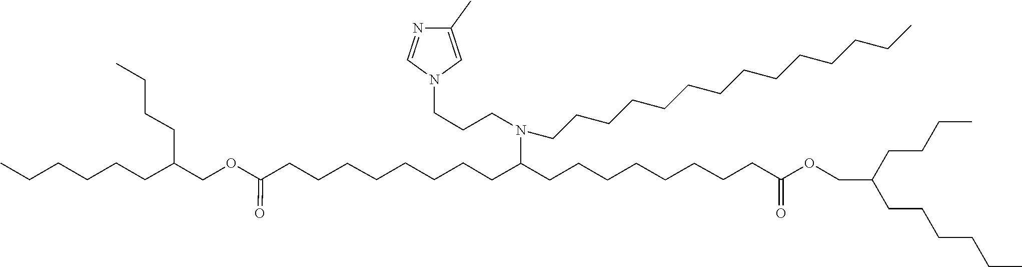 [SMILES: CCCCCCCCCCCCCCN(CCCn1cnc(C)c1)C(CCCCCCCCC(=O)OCC(CCCC)CCCCCC)CCCCCCCCC(=O)OCC(CCCC)CCCCCC] |
| 12 | 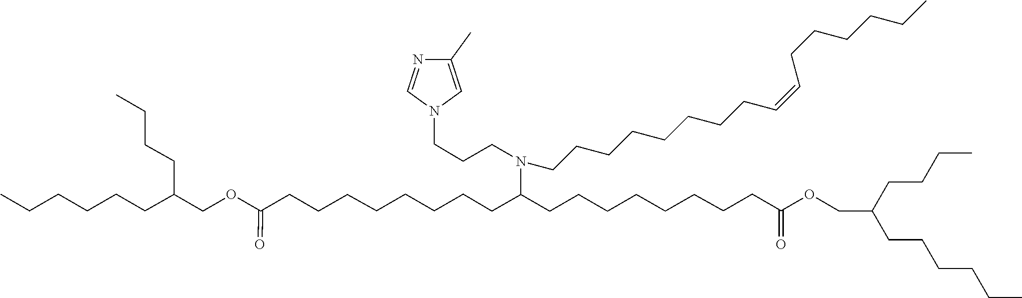 [SMILES: CCCCCC/C=C\CCCCCCCCN(CCCn1cnc(C)c1)C(CCCCCCCCC(=O)OCC(CCCC)CCCCCC)CCCCCCCCC(=O)OCC(CCCC)CCCCCC] |
| 13 | 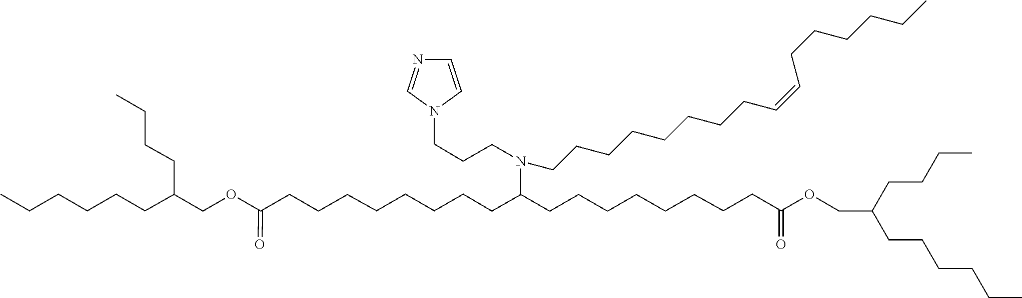 [SMILES: CCCCCC/C=C\CCCCCCCCN(CCCn1ccnc1)C(CCCCCCCCC(=O)OCC(CCCC)CCCCCC)CCCCCCCCC(=O)OCC(CCCC)CCCCCC] |
| 14 | 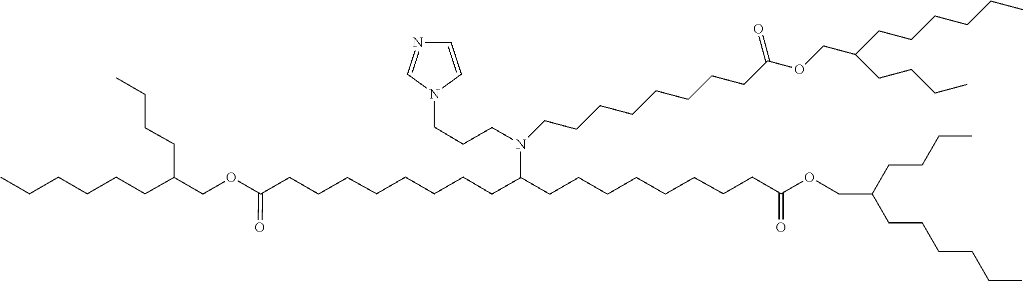 [SMILES: CCCCCCC(CCCC)COC(=O)CCCCCCCCC(CCCCCCCCC(=O)OCC(CCCC)CCCCCC)N(CCCCCCCCC(=O)OCC(CCCC)CCCCCC)CCCn1ccnc1] |
| 15 | 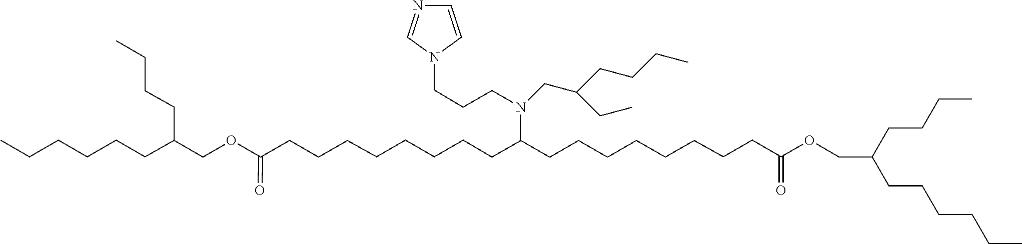 [SMILES: CCCCCCC(CCCC)COC(=O)CCCCCCCCC(CCCCCCCCC(=O)OCC(CCCC)CCCCCC)N(CCCn1ccnc1)CC(CC)CCCC] |
| 16 | 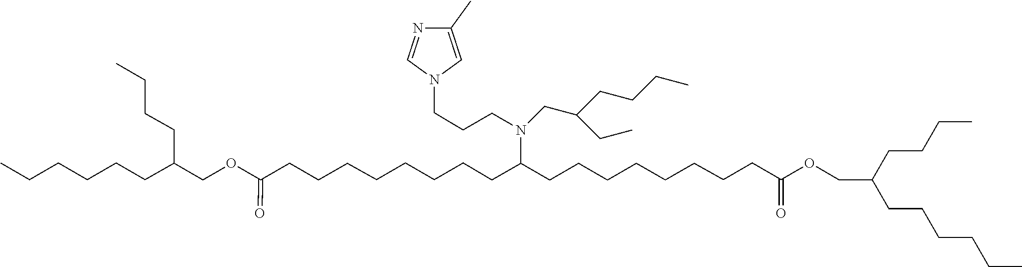 [SMILES: CCCCCCC(CCCC)COC(=O)CCCCCCCCC(CCCCCCCCC(=O)OCC(CCCC)CCCCCC)N(CCCn1cnc(C)c1)CC(CC)CCCC] |
| 17 | 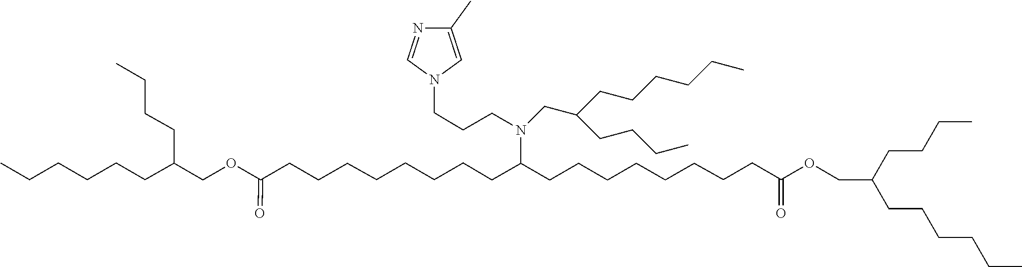 [SMILES: CCCCCCC(CCCC)COC(=O)CCCCCCCCC(CCCCCCCCC(=O)OCC(CCCC)CCCCCC)N(CCCn1cnc(C)c1)CC(CCCC)CCCCCC] |
| 18 | 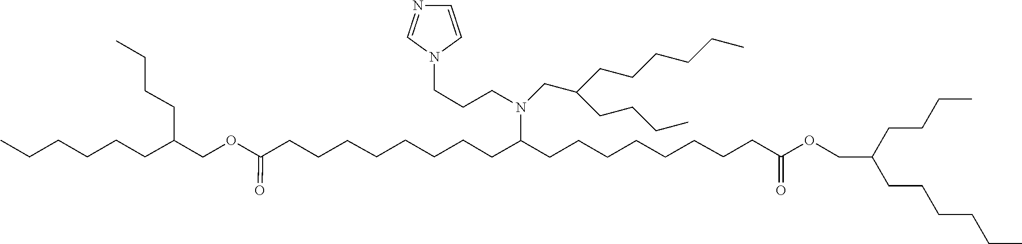 [SMILES: CCCCCCC(CCCC)COC(=O)CCCCCCCCC(CCCCCCCCC(=O)OCC(CCCC)CCCCCC)N(CCCn1ccnc1)CC(CCCC)CCCCCC] |
| 19 | 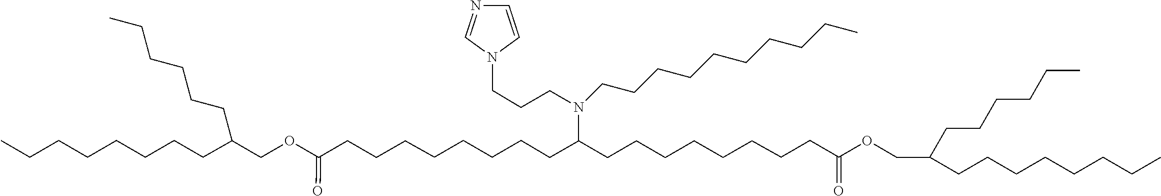 [SMILES: CCCCCCCCCCN(CCCn1ccnc1)C(CCCCCCCCC(=O)OCC(CCCCCC)CCCCCCCC)CCCCCCCCC(=O)OCC(CCCCCC)CCCCCCCC] |
| 20 | 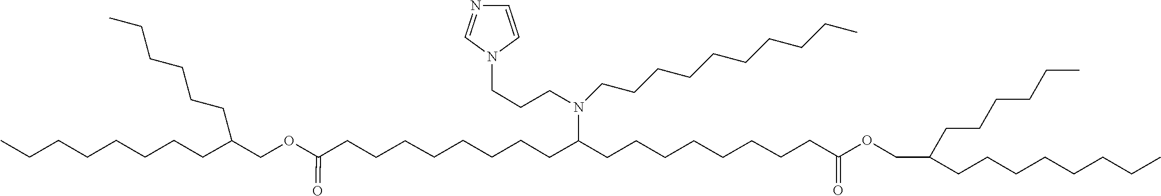 [SMILES: CCCCCCCCCCN(CCCn1ccnc1)C(CCCCCCCCC(=O)OCC(CCCCCC)CCCCCCCC)CCCCCCCCC(=O)OCC(CCCCCC)CCCCCCCC] |
| 21 | 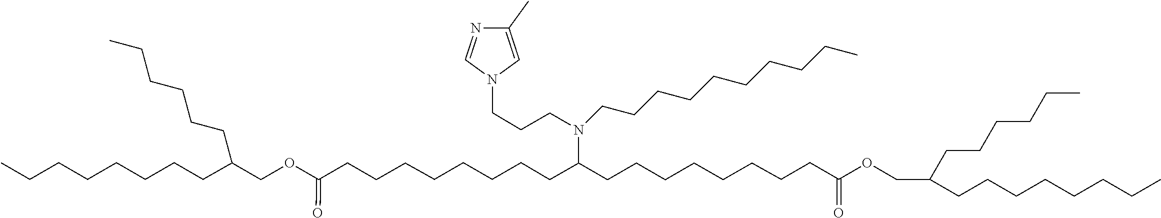 [SMILES: CCCCCCCCCCN(CCCn1cnc(C)c1)C(CCCCCCCCC(=O)OCC(CCCCCC)CCCCCCCC)CCCCCCCCC(=O)OCC(CCCCCC)CCCCCCCC] |
| 22 | 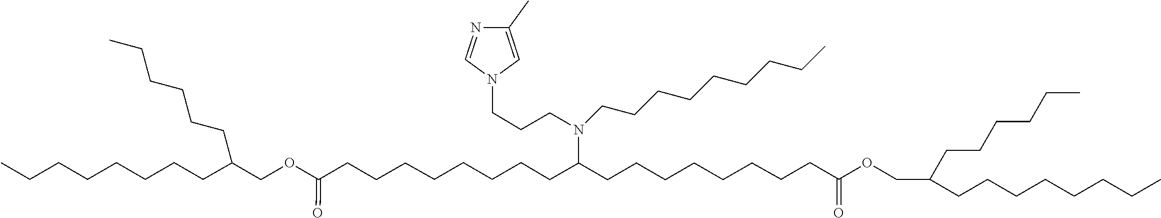 [SMILES: CCCCCCCCCN(CCCn1cnc(C)c1)C(CCCCCCCCC(=O)OCC(CCCCCC)CCCCCCCC)CCCCCCCCC(=O)OCC(CCCCCC)CCCCCCCC] |
| 23 | 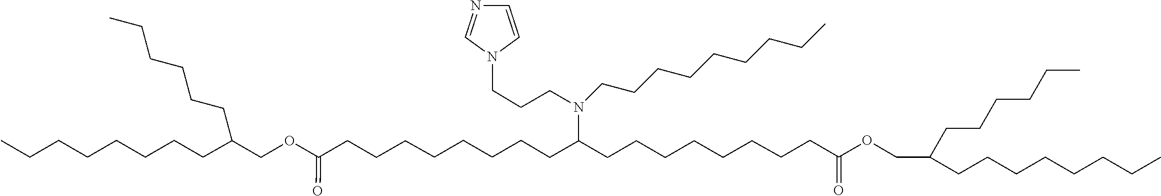 [SMILES: CCCCCCCCCN(CCCn1ccnc1)C(CCCCCCCCC(=O)OCC(CCCCCC)CCCCCCCC)CCCCCCCCC(=O)OCC(CCCCCC)CCCCCCCC] |
| 24 | 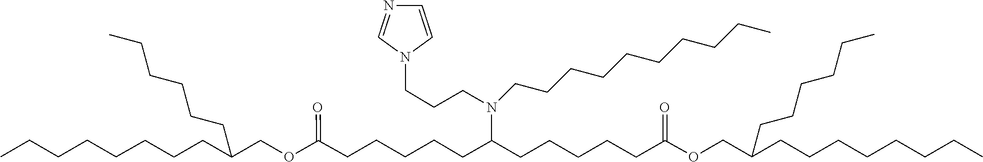 [SMILES: CCCCCCCCCCN(CCCn1ccnc1)C(CCCCCC(=O)OCC(CCCCCC)CCCCCCCC)CCCCCC(=O)OCC(CCCCCC)CCCCCCCC] |
| 25 | 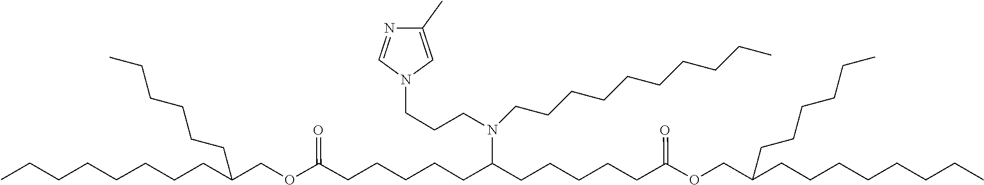 [SMILES: CCCCCCCCCCN(CCCn1cnc(C)c1)C(CCCCCC(=O)OCC(CCCCCC)CCCCCCCC)CCCCCC(=O)OCC(CCCCCC)CCCCCCCC] |
| 26 | 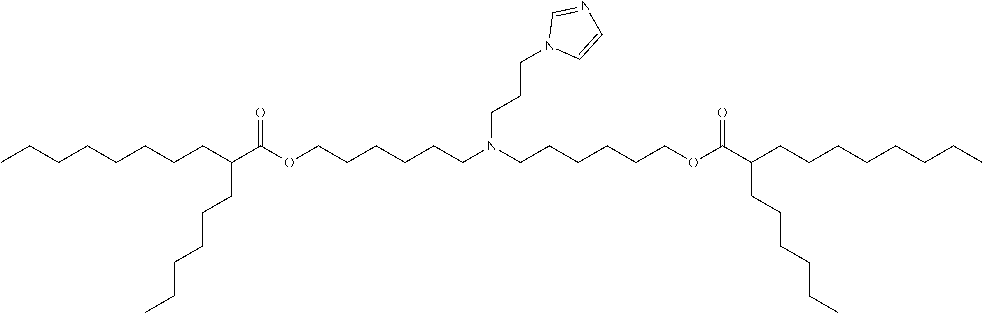 [SMILES: CCCCCCCCC(CCCCCC)C(=O)OCCCCCCN(CCCCCCOC(=O)C(CCCCCC)CCCCCCCC)CCCn1ccnc1] |
| 27 | 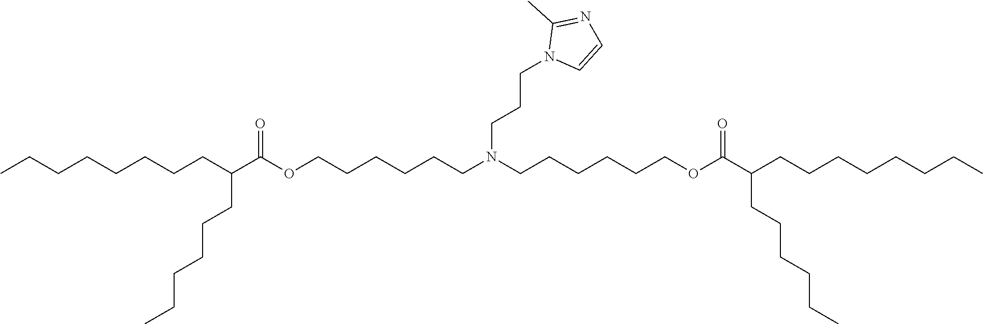 [SMILES: CCCCCCCCC(CCCCCC)C(=O)OCCCCCCN(CCCCCCOC(=O)C(CCCCCC)CCCCCCCC)CCCn1ccnc1C] |
| 28 | 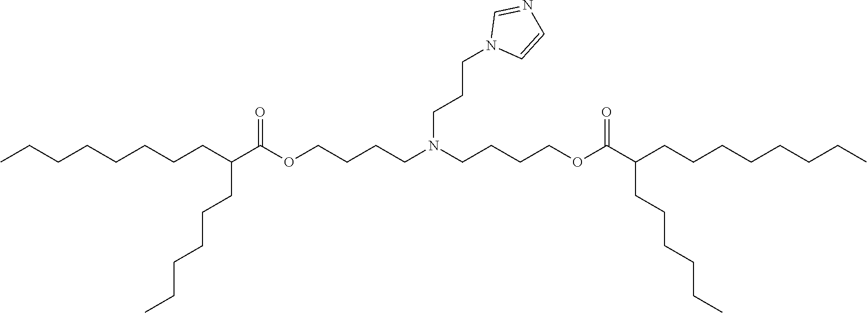 [SMILES: CCCCCCCCC(CCCCCC)C(=O)OCCCCN(CCCCOC(=O)C(CCCCCC)CCCCCCCC)CCCn1ccnc1] |
| 29 | 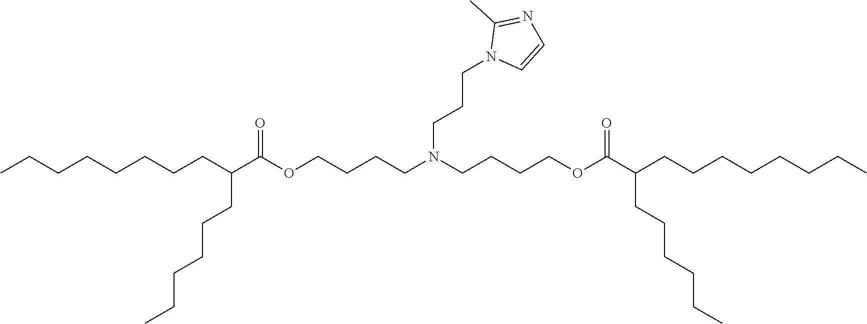 [SMILES: CCCCCCCCC(CCCCCC)C(=O)OCCCCN(CCCCOC(=O)C(CCCCCC)CCCCCCCC)CCCn1ccnc1C] |
| 30 | 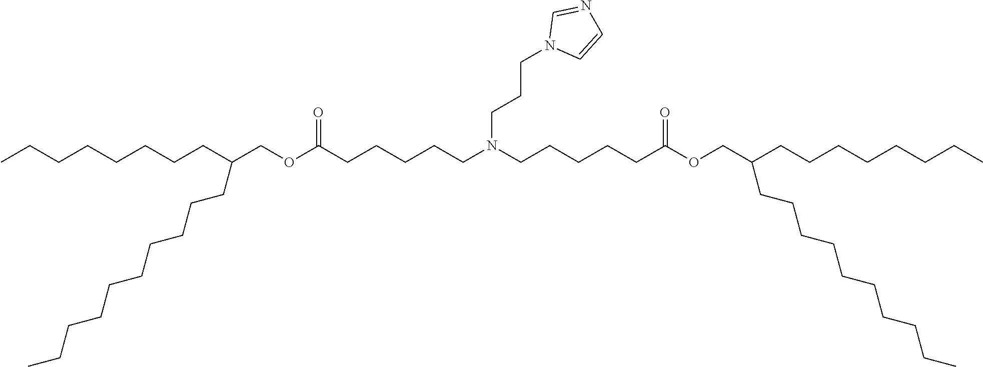 [SMILES: CCCCCCCCCCC(CCCCCCCC)COC(=O)CCCCCN(CCCCCC(=O)OCC(CCCCCCCC)CCCCCCCCCC)CCCn1ccnc1] |
| 31 | 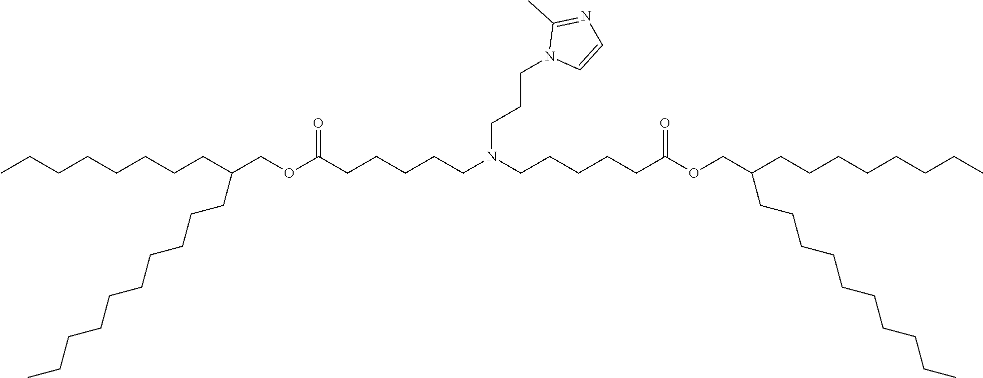 [SMILES: CCCCCCCCCCC(CCCCCCCC)COC(=O)CCCCCN(CCCCCC(=O)OCC(CCCCCCCC)CCCCCCCCCC)CCCn1ccnc1C] |
| 32 | 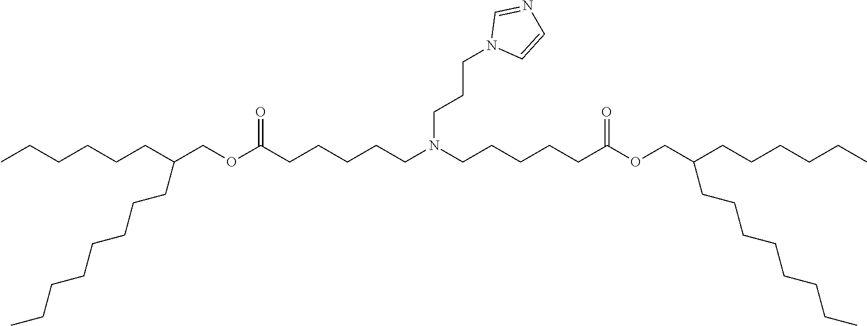 [SMILES: CCCCCCCCC(CCCCCC)COC(=O)CCCCCN(CCCCCC(=O)OCC(CCCCCC)CCCCCCCC)CCCn1ccnc1] |
| 33 | 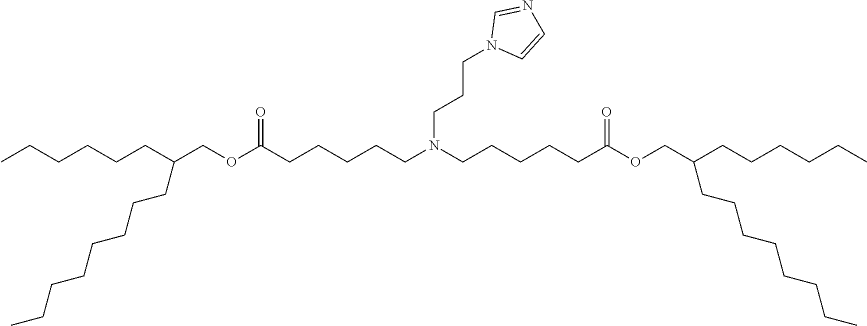 [SMILES: CCCCCCCCC(CCCCCC)COC(=O)CCCCCN(CCCCCC(=O)OCC(CCCCCC)CCCCCCCC)CCCn1ccnc1] |
| 34 | 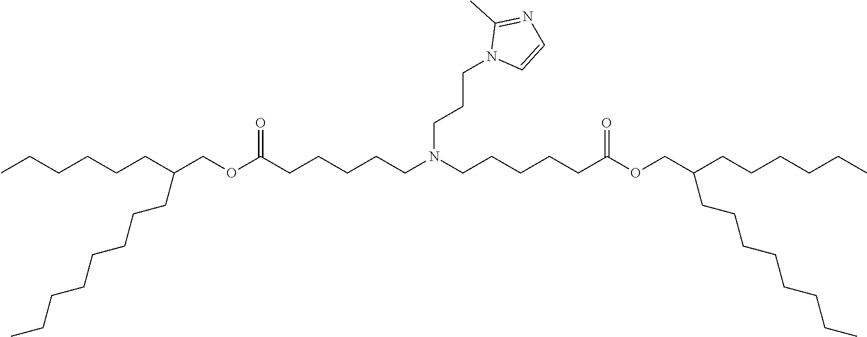 [SMILES: CCCCCCCCC(CCCCCC)COC(=O)CCCCCN(CCCCCC(=O)OCC(CCCCCC)CCCCCCCC)CCCn1ccnc1C] |
| 35 | 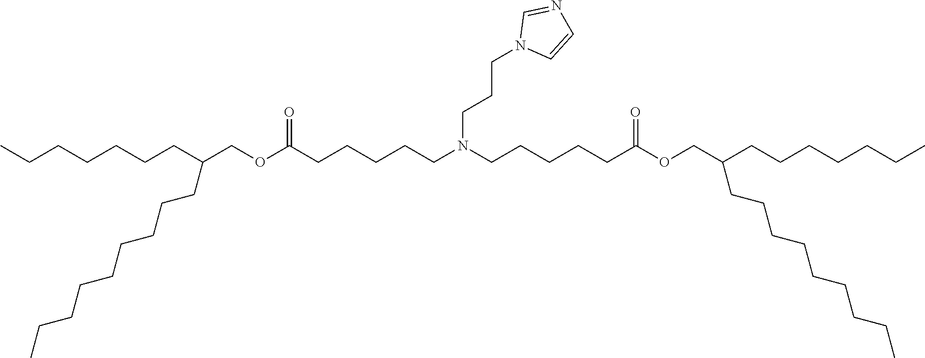 [SMILES: CCCCCCCCCC(CCCCCCC)COC(=O)CCCCCN(CCCCCC(=O)OCC(CCCCCCC)CCCCCCCCC)CCCn1ccnc1] |
| 36 | 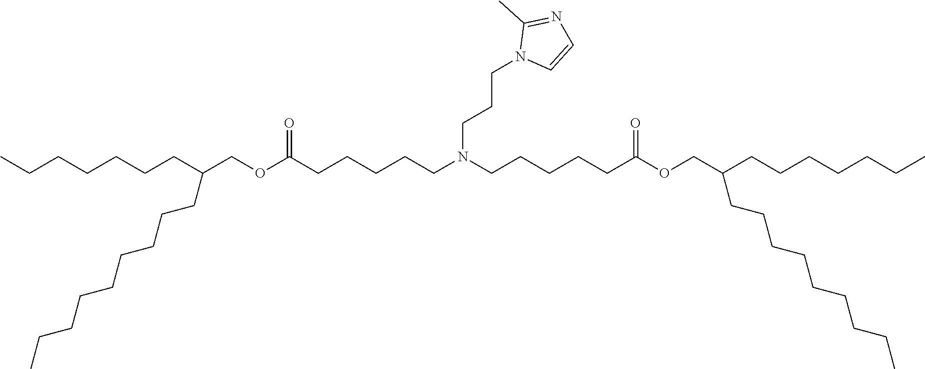 [SMILES: CCCCCCCCCC(CCCCCCC)COC(=O)CCCCCN(CCCCCC(=O)OCC(CCCCCCC)CCCCCCCCC)CCCn1ccnc1C] |
| 37 | 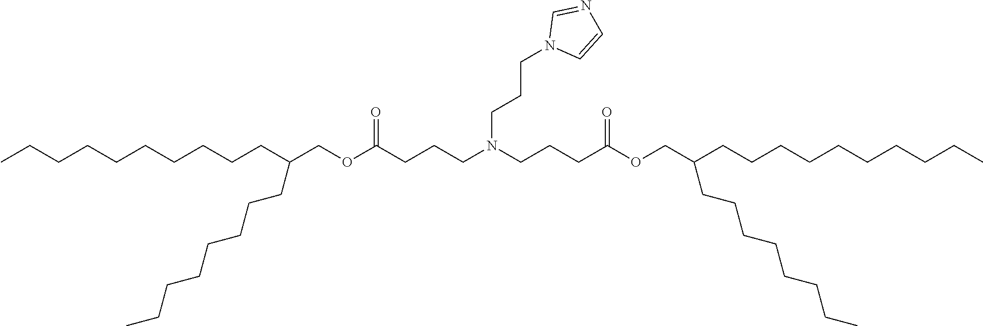 [SMILES: CCCCCCCCCCC(CCCCCCCC)COC(=O)CCCN(CCCC(=O)OCC(CCCCCCCC)CCCCCCCCCC)CCCn1ccnc1] |
| 38 | 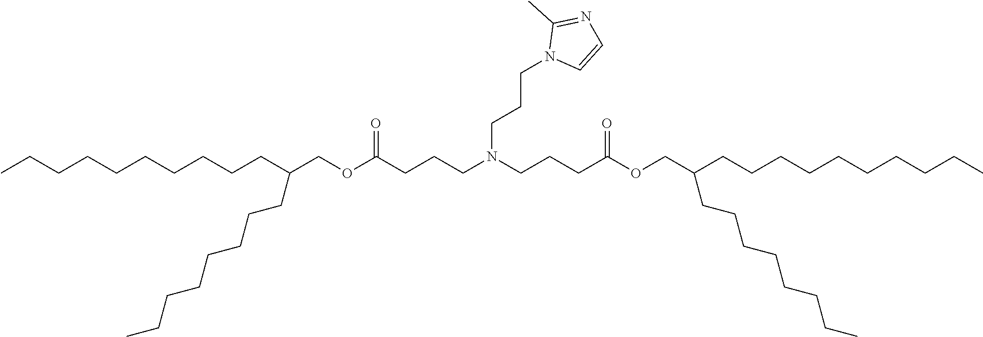 [SMILES: CCCCCCCCCCC(CCCCCCCC)COC(=O)CCCN(CCCC(=O)OCC(CCCCCCCC)CCCCCCCCCC)CCCn1ccnc1C] |
| 39 | 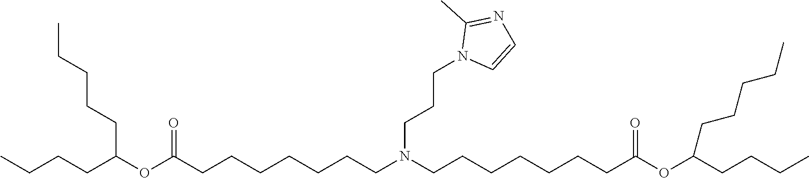 [SMILES: CCCCCC(CCCC)OC(=O)CCCCCCCN(CCCCCCCC(=O)OC(CCCC)CCCCC)CCCn1ccnc1C] |
| 40 | 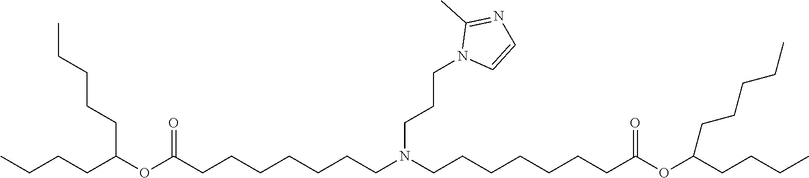 [SMILES: CCCCCC(CCCC)OC(=O)CCCCCCCN(CCCCCCCC(=O)OC(CCCC)CCCCC)CCCn1ccnc1C] |
| 41 | 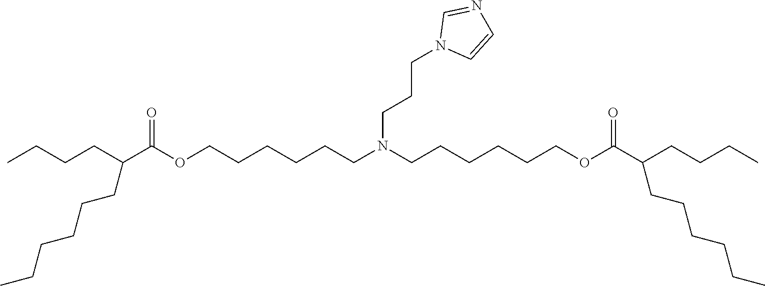 [SMILES: CCCCCCC(CCCC)C(=O)OCCCCCCN(CCCCCCOC(=O)C(CCCC)CCCCCC)CCCn1ccnc1] |
| 42 | 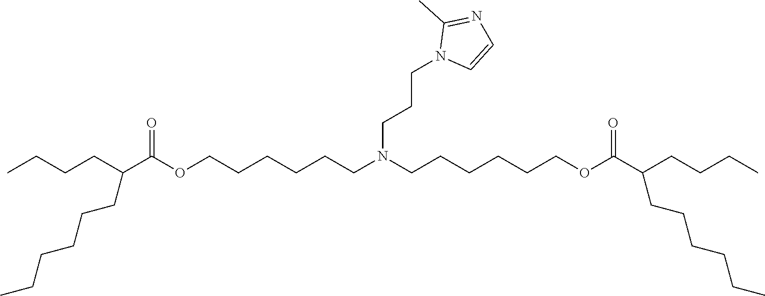 [SMILES: CCCCCCC(CCCC)C(=O)OCCCCCCN(CCCCCCOC(=O)C(CCCC)CCCCCC)CCCn1ccnc1C] |
| 43 | 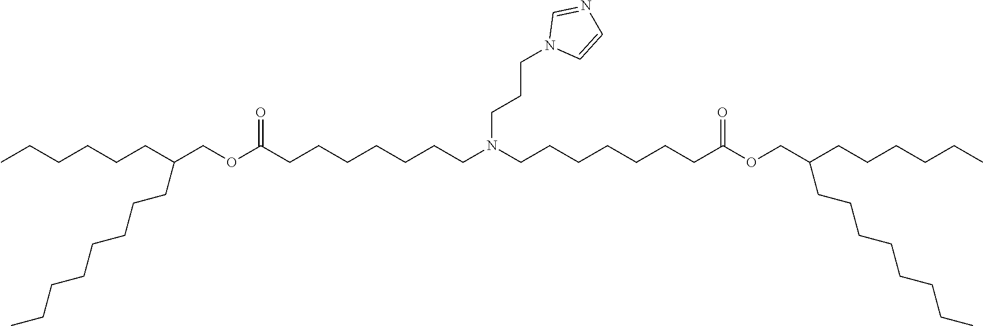 [SMILES: CCCCCCCCC(CCCCCC)COC(=O)CCCCCCCN(CCCCCCCC(=O)OCC(CCCCCC)CCCCCCCC)CCCn1ccnc1] |
| 44 | 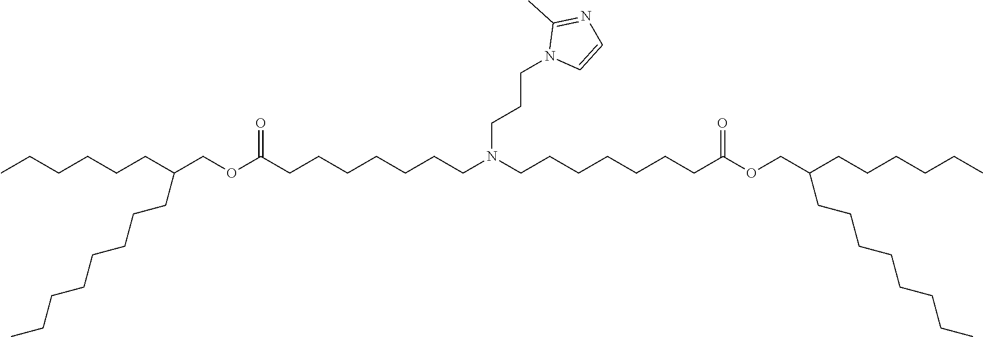 [SMILES: CCCCCCCCC(CCCCCC)COC(=O)CCCCCCCN(CCCCCCCC(=O)OCC(CCCCCC)CCCCCCCC)CCCn1ccnc1C] |
| 45 | 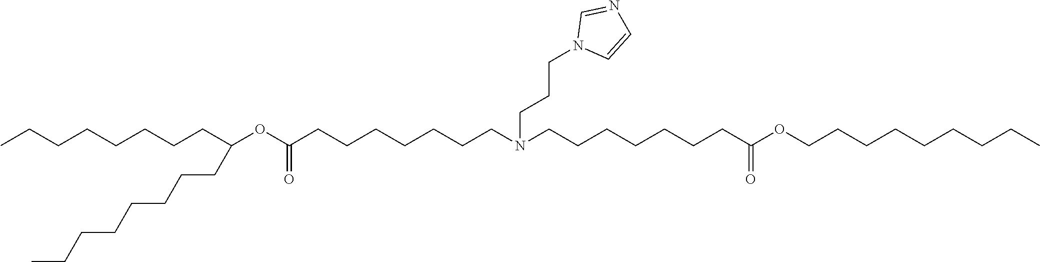 [SMILES: CCCCCCCCCOC(=O)CCCCCCCN(CCCCCCCC(=O)OC(CCCCCCCC)CCCCCCCC)CCCn1ccnc1] |
| 46 | 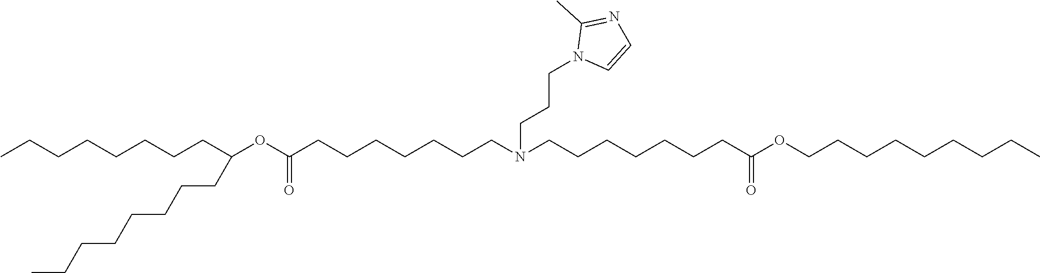 [SMILES: CCCCCCCCCOC(=O)CCCCCCCN(CCCCCCCC(=O)OC(CCCCCCCC)CCCCCCCC)CCCn1ccnc1C] |
| 47 | 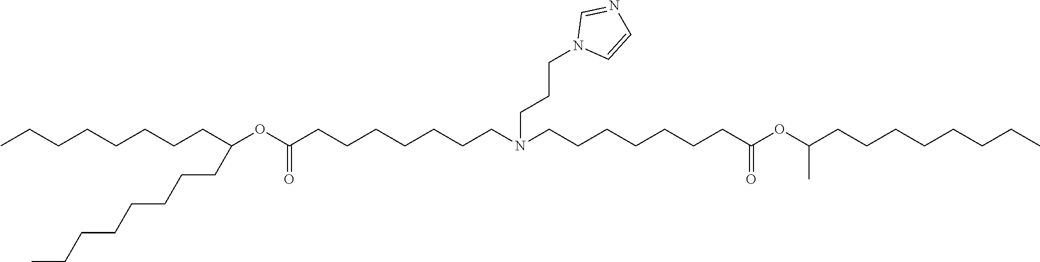 [SMILES: CCCCCCCCC(C)OC(=O)CCCCCCCN(CCCCCCCC(=O)OC(CCCCCCCC)CCCCCCCC)CCCn1ccnc1] |
| 48 | 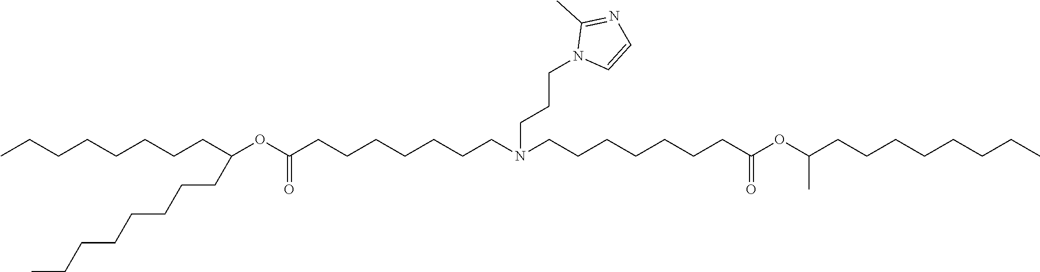 [SMILES: CCCCCCCCC(C)OC(=O)CCCCCCCN(CCCCCCCC(=O)OC(CCCCCCCC)CCCCCCCC)CCCn1ccnc1C] |
| 49 | 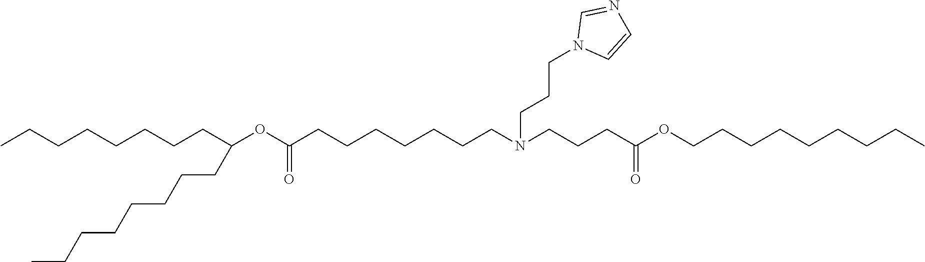 [SMILES: CCCCCCCCCOC(=O)CCCN(CCCCCCCC(=O)OC(CCCCCCCC)CCCCCCCC)CCCn1ccnc1] |
| 50 | 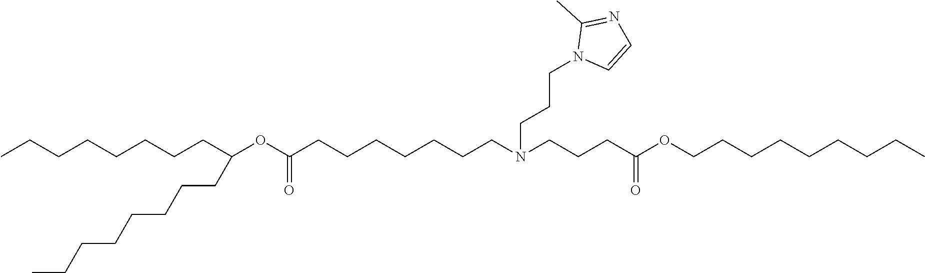 [SMILES: CCCCCCCCCOC(=O)CCCN(CCCCCCCC(=O)OC(CCCCCCCC)CCCCCCCC)CCCn1ccnc1C] |
| 51 | 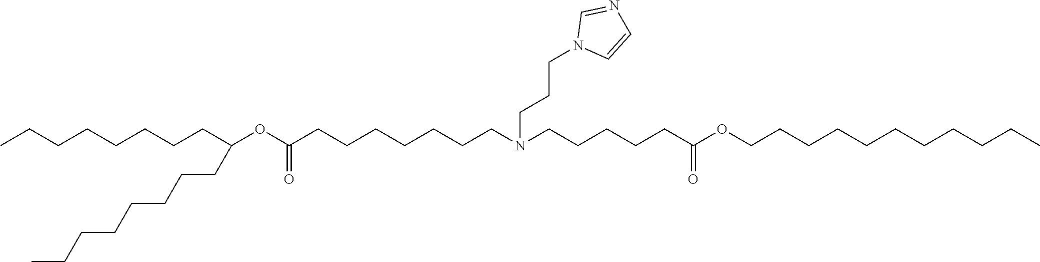 [SMILES: CCCCCCCCCCCOC(=O)CCCCCN(CCCCCCCC(=O)OC(CCCCCCCC)CCCCCCCC)CCCn1ccnc1] |
| 52 | 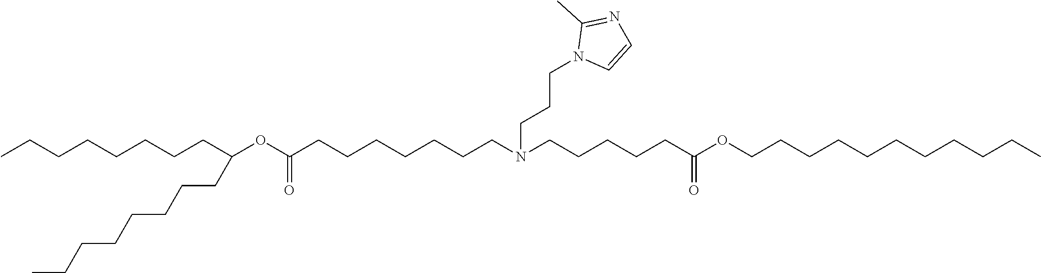 [SMILES: CCCCCCCCCCCOC(=O)CCCCCN(CCCCCCCC(=O)OC(CCCCCCCC)CCCCCCCC)CCCn1ccnc1C] |
| 53 | 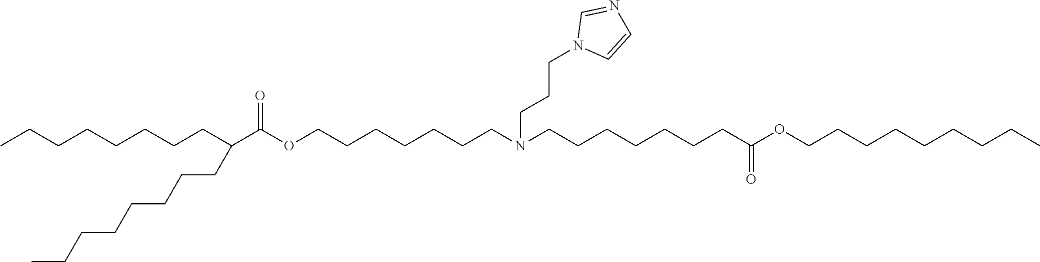 [SMILES: CCCCCCCCCOC(=O)CCCCCCCN(CCCCCCCOC(=O)C(CCCCCCCC)CCCCCCCC)CCCn1ccnc1] |
| 54 | 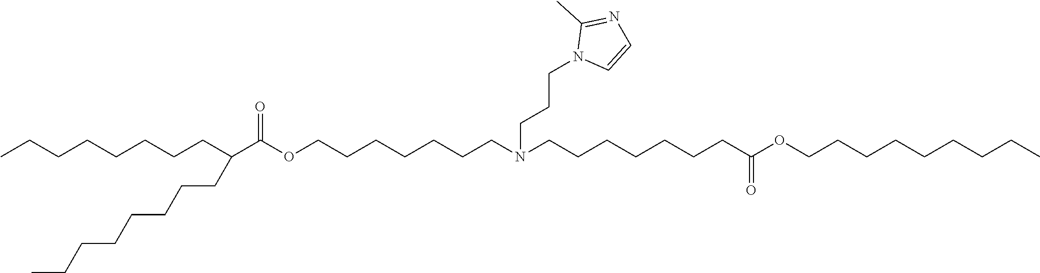 [SMILES: CCCCCCCCCOC(=O)CCCCCCCN(CCCCCCCOC(=O)C(CCCCCCCC)CCCCCCCC)CCCn1ccnc1C] |
| 55 | 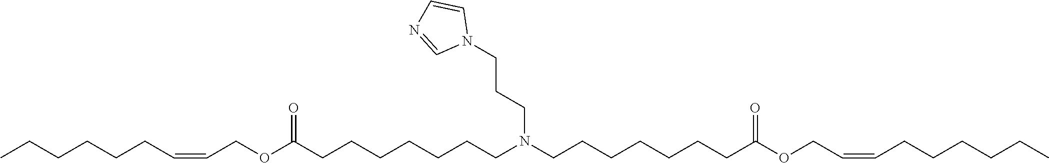 [SMILES: CCCCCC/C=C\COC(=O)CCCCCCCN(CCCCCCCC(=O)OC/C=C\CCCCCC)CCCn1ccnc1] |
| 56 | 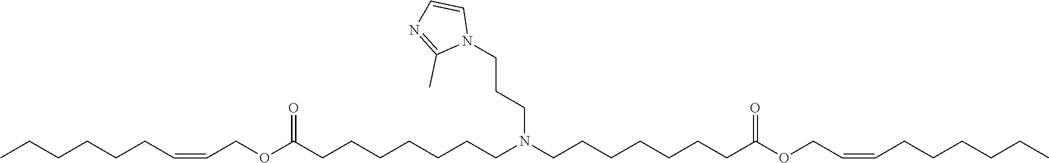 [SMILES: CCCCCC/C=C\COC(=O)CCCCCCCN(CCCCCCCC(=O)OC/C=C\CCCCCC)CCCn1ccnc1C] |
| 57 | 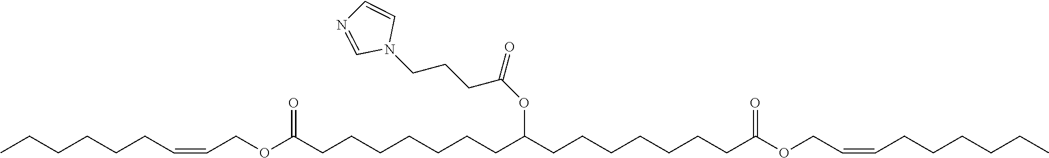 [SMILES: CCCCCC/C=C\COC(=O)CCCCCCCC(CCCCCCCC(=O)OC/C=C\CCCCCC)OC(=O)CCCn1ccnc1] |
| 58 | 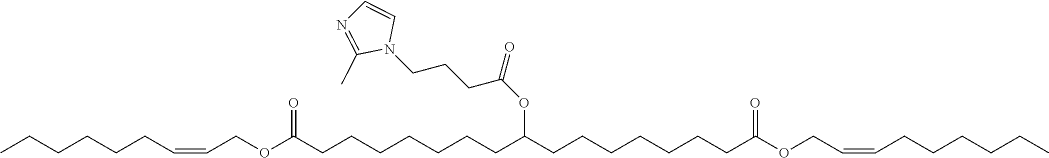 [SMILES: CCCCCC/C=C\COC(=O)CCCCCCCC(CCCCCCCC(=O)OC/C=C\CCCCCC)OC(=O)CCCn1ccnc1C] |
| 59 | 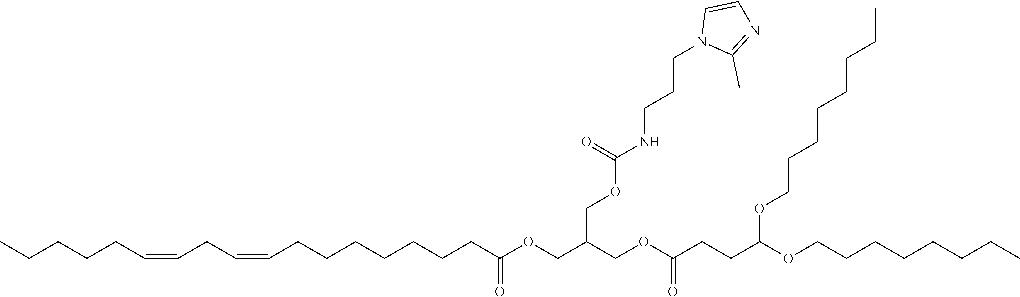 [SMILES: CCCCC/C=C\C/C=C\CCCCCCCC(=O)OCC(COC(=O)CCC(OCCCCCCCC)OCCCCCCCC)COC(=O)NCCCn1ccnc1C] |
| 60 | 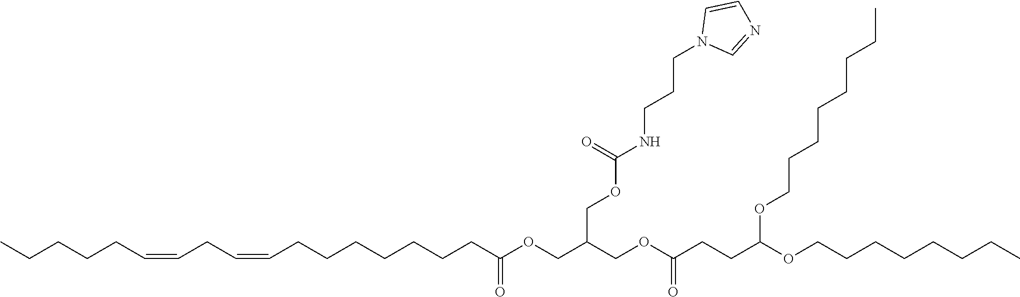 [SMILES: CCCCC/C=C\C/C=C\CCCCCCCC(=O)OCC(COC(=O)CCC(OCCCCCCCC)OCCCCCCCC)COC(=O)NCCCn1ccnc1] |
| 61 | 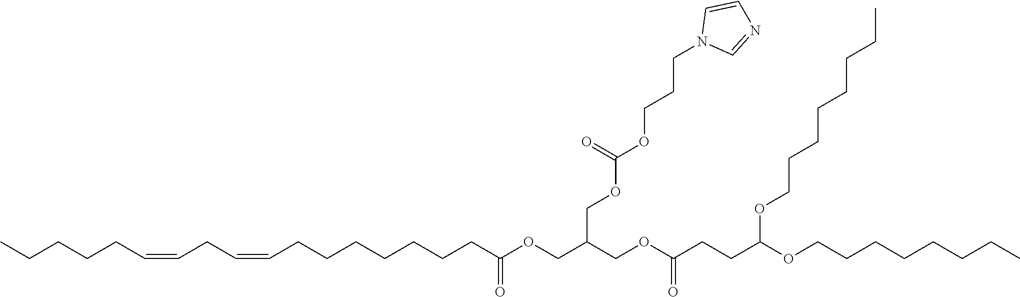 [SMILES: CCCCC/C=C\C/C=C\CCCCCCCC(=O)OCC(COC(=O)CCC(OCCCCCCCC)OCCCCCCCC)COC(=O)OCCCn1ccnc1] |
| 62 | 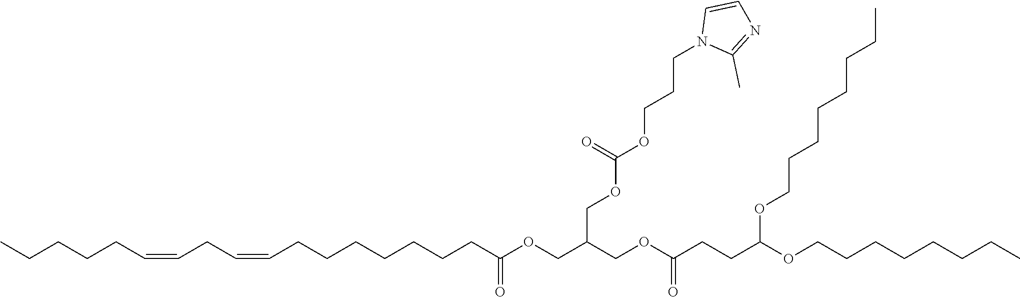 [SMILES: CCCCC/C=C\C/C=C\CCCCCCCC(=O)OCC(COC(=O)CCC(OCCCCCCCC)OCCCCCCCC)COC(=O)OCCCn1ccnc1C] |
| 63 | 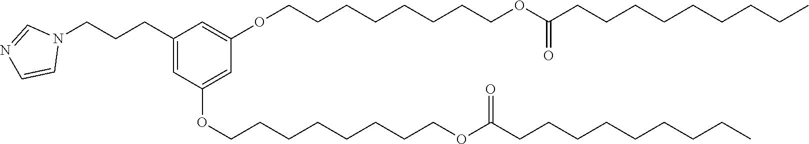 [SMILES: CCCCCCCCCC(=O)OCCCCCCCCOc1cc(CCCn2ccnc2)cc(OCCCCCCCCOC(=O)CCCCCCCCC)c1] |
| 64 | 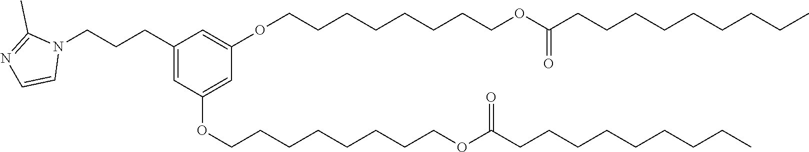 [SMILES: CCCCCCCCCC(=O)OCCCCCCCCOc1cc(CCCn2ccnc2C)cc(OCCCCCCCCOC(=O)CCCCCCCCC)c1] |
| 65 | 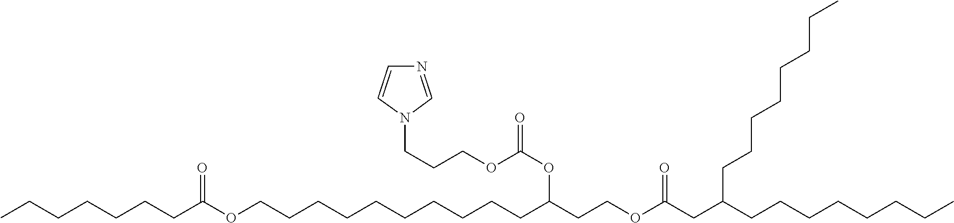 [SMILES: CCCCCCCCC(CCCCCCCC)CC(=O)OCCC(CCCCCCCCCCOC(=O)CCCCCCC)OC(=O)OCCCn1ccnc1] |
| 66 | 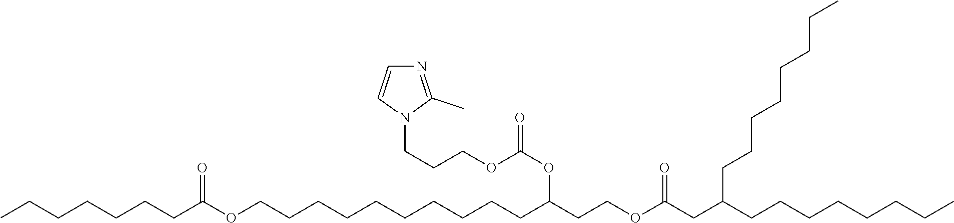 [SMILES: CCCCCCCCC(CCCCCCCC)CC(=O)OCCC(CCCCCCCCCCOC(=O)CCCCCCC)OC(=O)OCCCn1ccnc1C] |
| 67 | 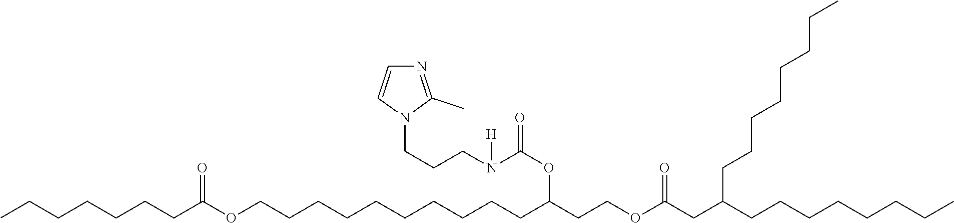 [SMILES: CCCCCCCCC(CCCCCCCC)CC(=O)OCCC(CCCCCCCCCCOC(=O)CCCCCCC)OC(=O)NCCCn1ccnc1C] |
| 68 | 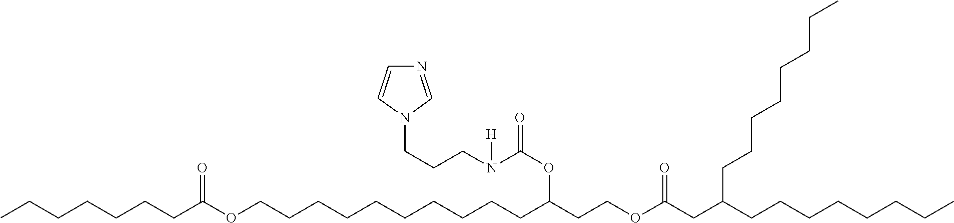 [SMILES: CCCCCCCCC(CCCCCCCC)CC(=O)OCCC(CCCCCCCCCCOC(=O)CCCCCCC)OC(=O)NCCCn1ccnc1] |
| 69 | 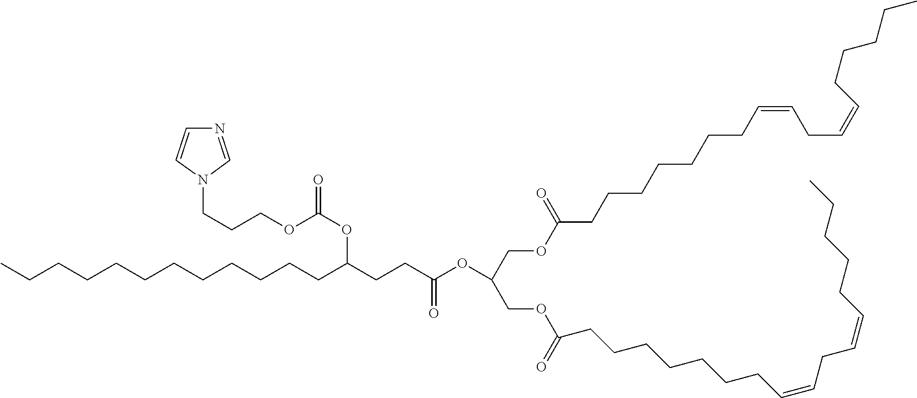 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCn1ccnc1] |
| 70 | 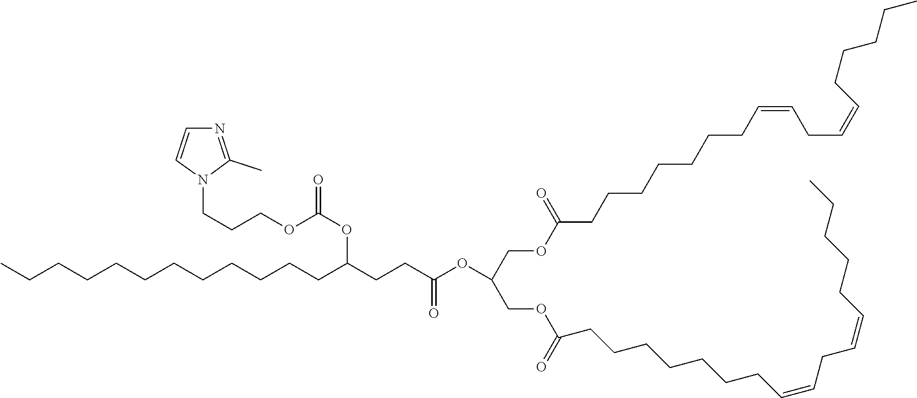 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCn1ccnc1C] |
| 71 | 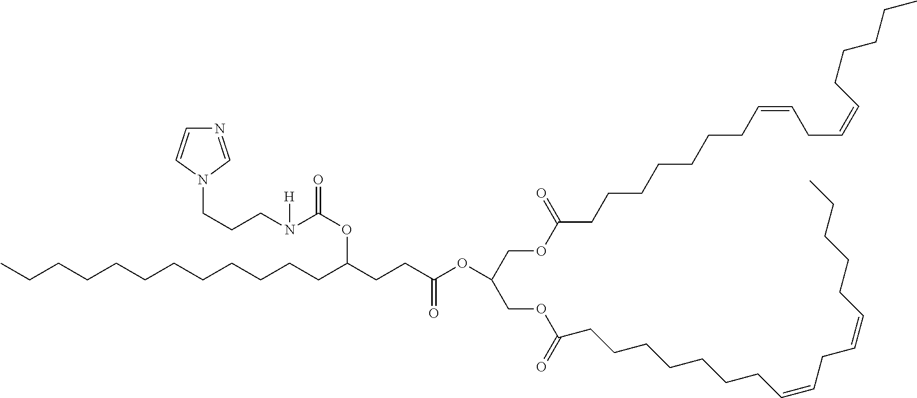 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)NCCCn1ccnc1] |
| 72 | 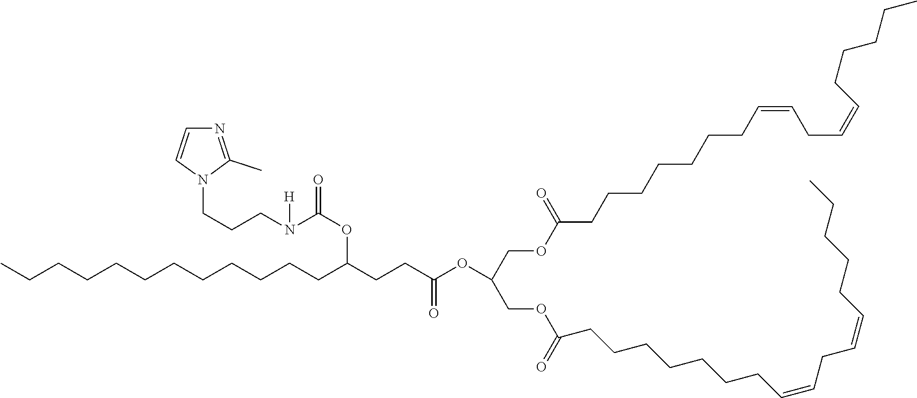 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)NCCCn1ccnc1C] |
| 73 | 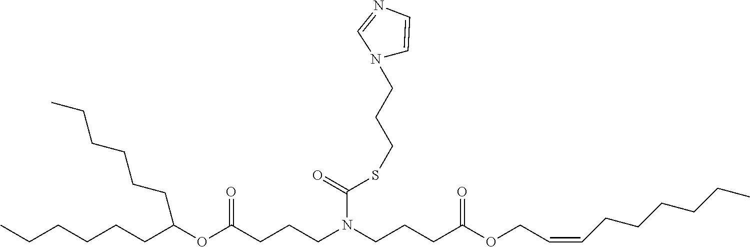 [SMILES: CCCCCC/C=C\COC(=O)CCCN(CCCC(=O)OC(CCCCCC)CCCCCC)C(=O)SCCCn1ccnc1] |
| 74 | 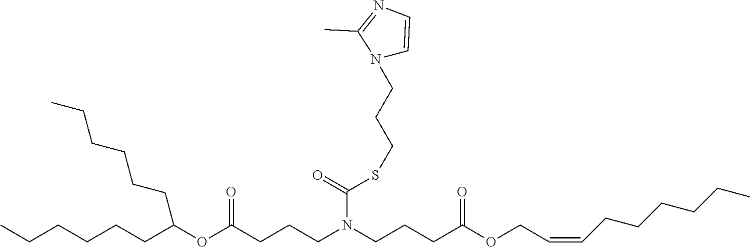 [SMILES: CCCCCC/C=C\COC(=O)CCCN(CCCC(=O)OC(CCCCCC)CCCCCC)C(=O)SCCCn1ccnc1C] |
| 75 | 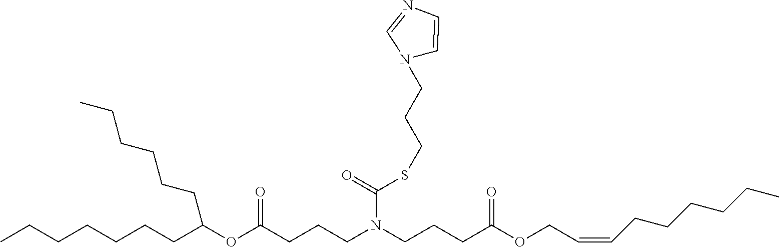 [SMILES: CCCCCC/C=C\COC(=O)CCCN(CCCC(=O)OC(CCCCCC)CCCCCCC)C(=O)SCCCn1ccnc1] |
| 76 | 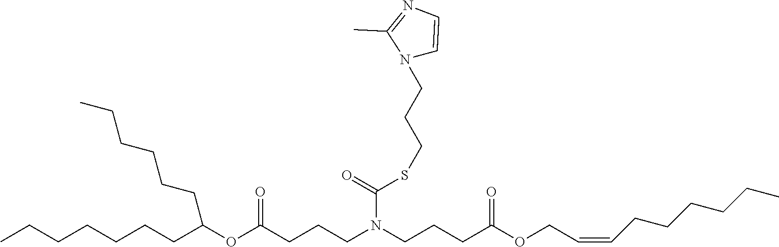 [SMILES: CCCCCC/C=C\COC(=O)CCCN(CCCC(=O)OC(CCCCCC)CCCCCCC)C(=O)SCCCn1ccnc1C] |
| 77 | 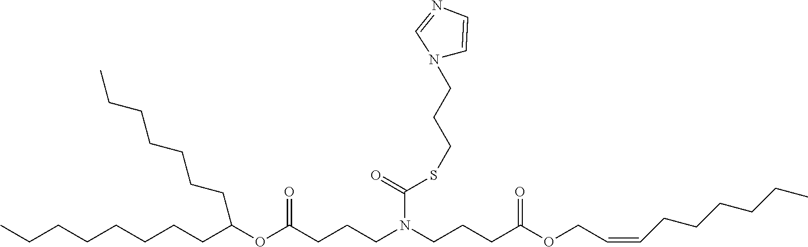 [SMILES: CCCCCC/C=C\COC(=O)CCCN(CCCC(=O)OC(CCCCCCC)CCCCCCCC)C(=O)SCCCn1ccnc1] |
| 78 | 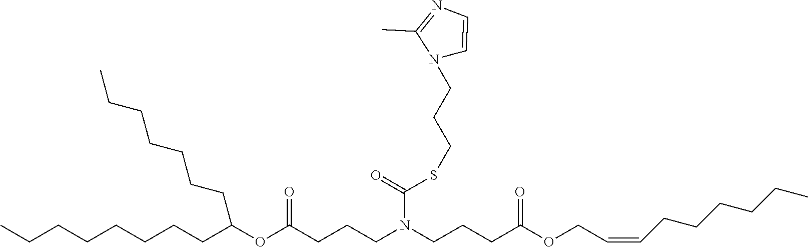 [SMILES: CCCCCC/C=C\COC(=O)CCCN(CCCC(=O)OC(CCCCCCC)CCCCCCCC)C(=O)SCCCn1ccnc1C] |
| 79 | 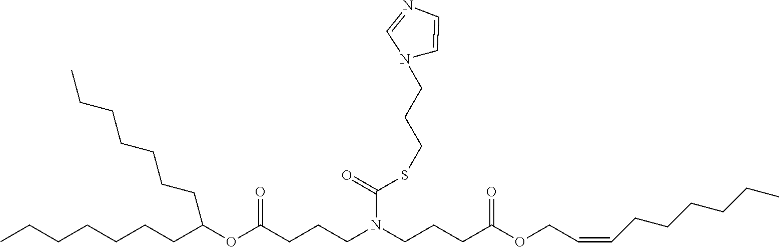 [SMILES: CCCCCC/C=C\COC(=O)CCCN(CCCC(=O)OC(CCCCCCC)CCCCCCC)C(=O)SCCCn1ccnc1] |
| 80 | 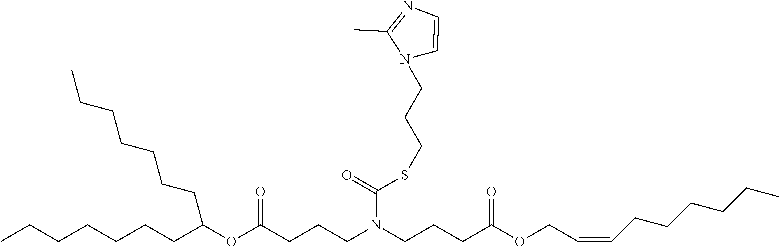 [SMILES: CCCCCC/C=C\COC(=O)CCCN(CCCC(=O)OC(CCCCCCC)CCCCCCC)C(=O)SCCCn1ccnc1C] |
| 81 | 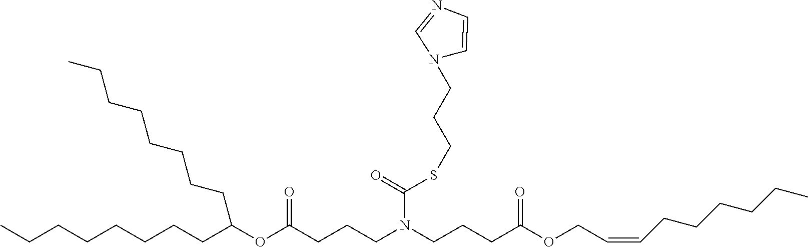 [SMILES: CCCCCC/C=C\COC(=O)CCCN(CCCC(=O)OC(CCCCCCCC)CCCCCCCC)C(=O)SCCCn1ccnc1] |
| 82 | 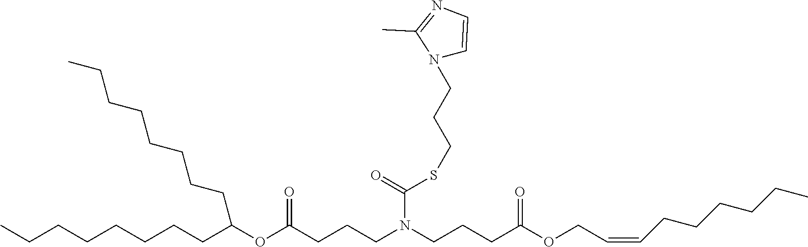 [SMILES: CCCCCC/C=C\COC(=O)CCCN(CCCC(=O)OC(CCCCCCCC)CCCCCCCC)C(=O)SCCCn1ccnc1C] |
| 83 | 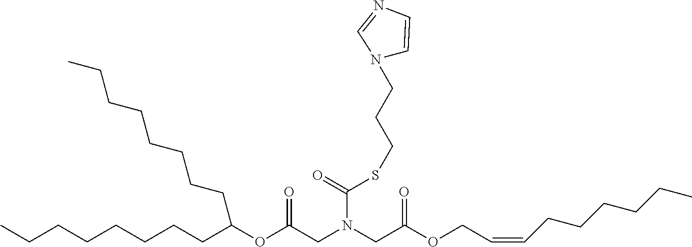 [SMILES: CCCCCC/C=C\COC(=O)CN(CC(=O)OC(CCCCCCCC)CCCCCCCC)C(=O)SCCCn1ccnc1] |
| 84 | 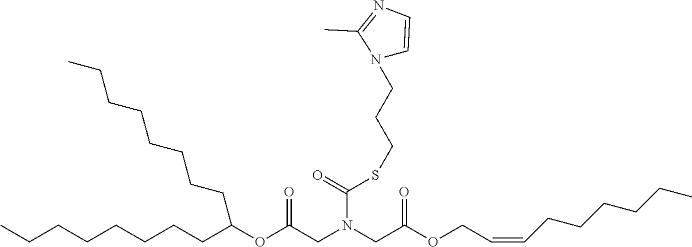 [SMILES: CCCCCC/C=C\COC(=O)CN(CC(=O)OC(CCCCCCCC)CCCCCCCC)C(=O)SCCCn1ccnc1C] |
| 85 | 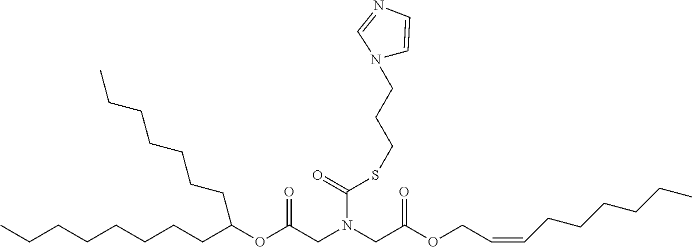 [SMILES: CCCCCC/C=C\COC(=O)CN(CC(=O)OC(CCCCCCC)CCCCCCCC)C(=O)SCCCn1ccnc1] |
| 86 | 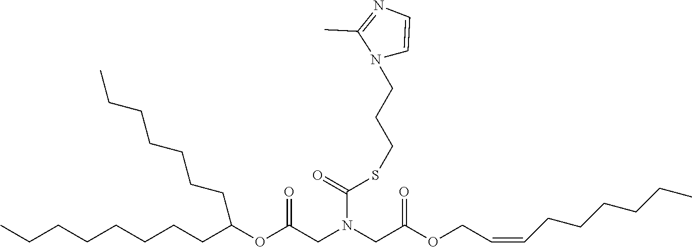 [SMILES: CCCCCC/C=C\COC(=O)CN(CC(=O)OC(CCCCCCC)CCCCCCCC)C(=O)SCCCn1ccnc1C] |
| 87 | 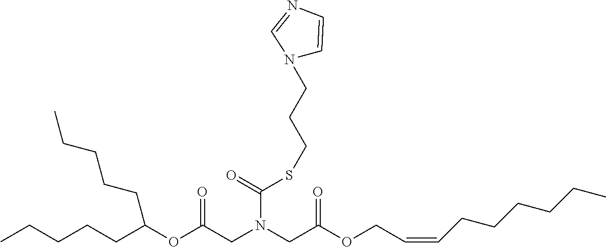 [SMILES: CCCCCC/C=C\COC(=O)CN(CC(=O)OC(CCCCC)CCCCC)C(=O)SCCCn1ccnc1] |
| 88 | 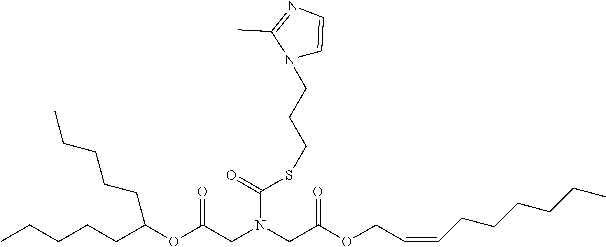 [SMILES: CCCCCC/C=C\COC(=O)CN(CC(=O)OC(CCCCC)CCCCC)C(=O)SCCCn1ccnc1C] |
| 89 | 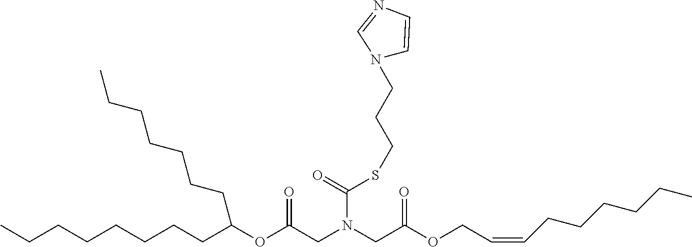 [SMILES: CCCCCC/C=C\COC(=O)CN(CC(=O)OC(CCCCCCC)CCCCCCCC)C(=O)SCCCn1ccnc1] |
| 90 | 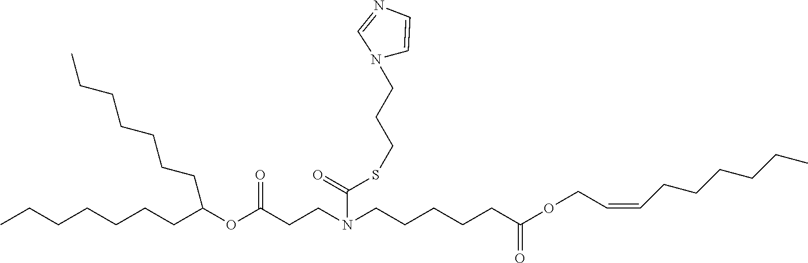 [SMILES: CCCCCC/C=C\COC(=O)CCCCCN(CCC(=O)OC(CCCCCCC)CCCCCCC)C(=O)SCCCn1ccnc1] |
| 91 | 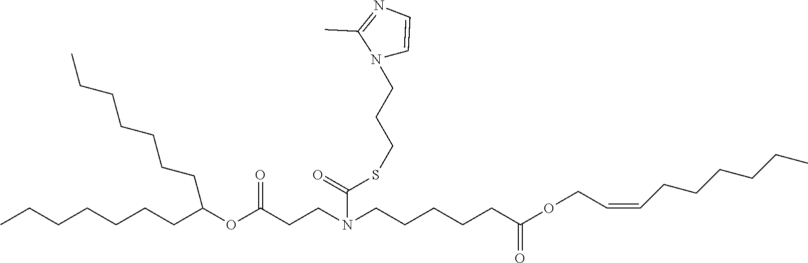 [SMILES: CCCCCC/C=C\COC(=O)CCCCCN(CCC(=O)OC(CCCCCCC)CCCCCCC)C(=O)SCCCn1ccnc1C] |
| 92 | 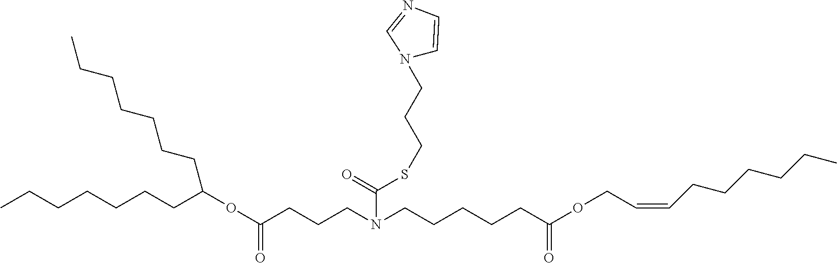 [SMILES: CCCCCC/C=C\COC(=O)CCCCCN(CCCC(=O)OC(CCCCCCC)CCCCCCC)C(=O)SCCCn1ccnc1] |
| 93 | 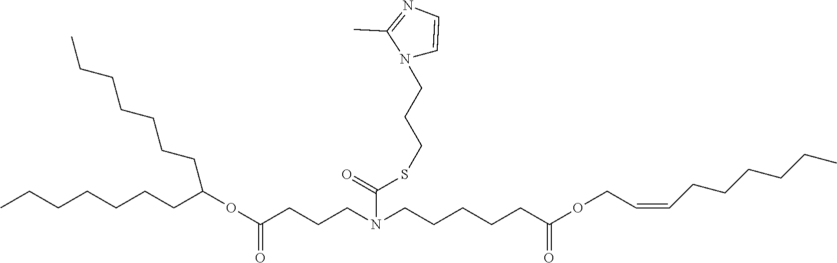 [SMILES: CCCCCC/C=C\COC(=O)CCCCCN(CCCC(=O)OC(CCCCCCC)CCCCCCC)C(=O)SCCCn1ccnc1C] |
| 94 | 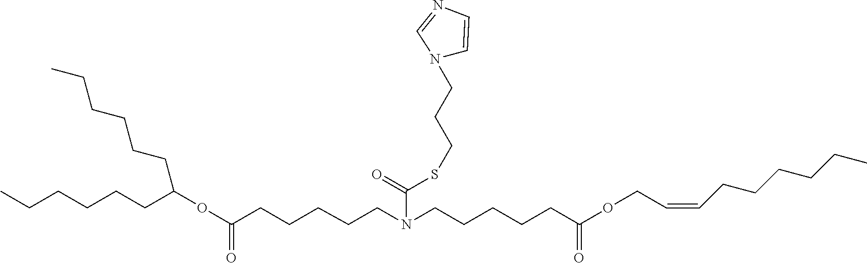 [SMILES: CCCCCC/C=C\COC(=O)CCCCCN(CCCCCC(=O)OC(CCCCCC)CCCCCC)C(=O)SCCCn1ccnc1] |
| 95 | 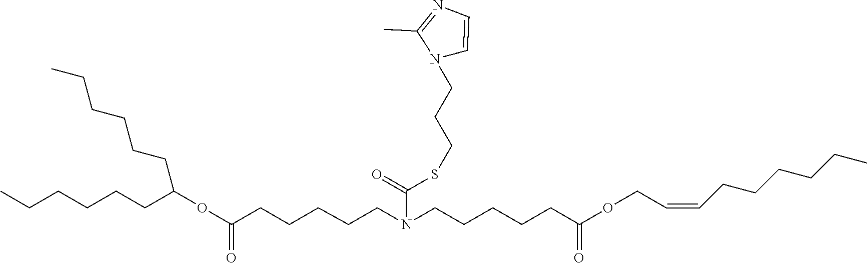 [SMILES: CCCCCC/C=C\COC(=O)CCCCCN(CCCCCC(=O)OC(CCCCCC)CCCCCC)C(=O)SCCCn1ccnc1C] |
| 96 | 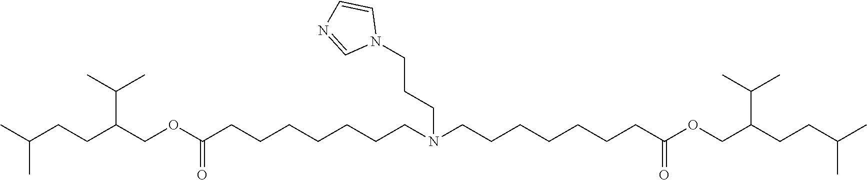 [SMILES: CC(C)CCC(COC(=O)CCCCCCCN(CCCCCCCC(=O)OCC(CCC(C)C)C(C)C)CCCn1ccnc1)C(C)C] |
| 97 | 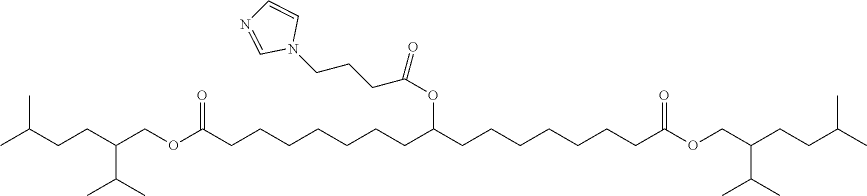 [SMILES: CC(C)CCC(COC(=O)CCCCCCCC(CCCCCCCC(=O)OCC(CCC(C)C)C(C)C)OC(=O)CCCn1ccnc1)C(C)C] |
| 98 | 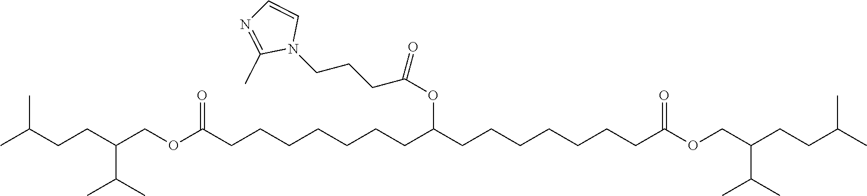 [SMILES: Cc1nccn1CCCC(=O)OC(CCCCCCCC(=O)OCC(CCC(C)C)C(C)C)CCCCCCCC(=O)OCC(CCC(C)C)C(C)C] |
| 99 | 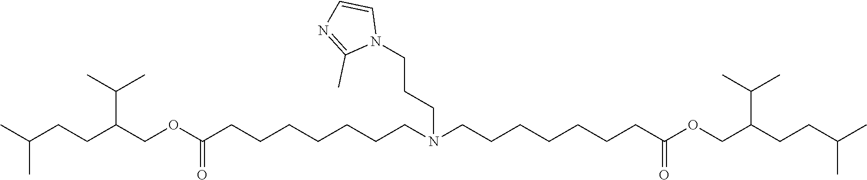 [SMILES: Cc1nccn1CCCN(CCCCCCCC(=O)OCC(CCC(C)C)C(C)C)CCCCCCCC(=O)OCC(CCC(C)C)C(C)C] |
| 100 | 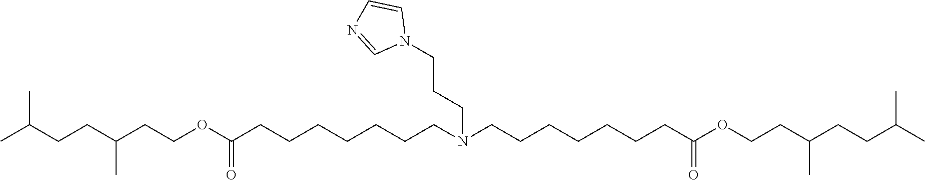 [SMILES: CC(C)CCC(C)CCOC(=O)CCCCCCCN(CCCCCCCC(=O)OCCC(C)CCC(C)C)CCCn1ccnc1] |
| 101 | 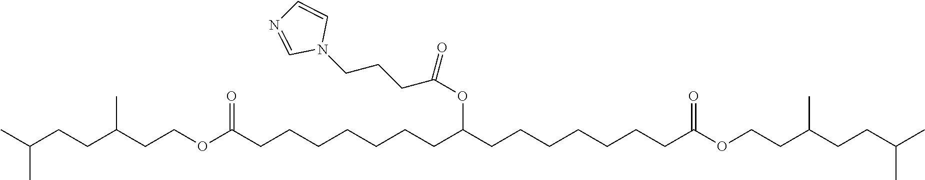 [SMILES: CC(C)CCC(C)CCOC(=O)CCCCCCCC(CCCCCCCC(=O)OCCC(C)CCC(C)C)OC(=O)CCCn1ccnc1] |
| 102 | 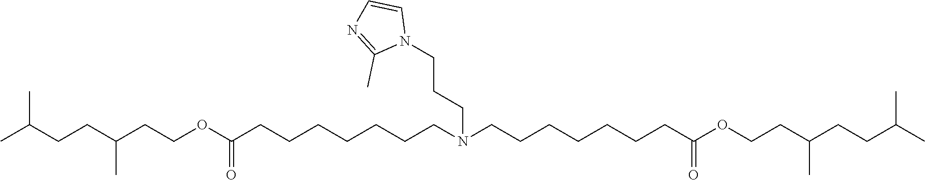 [SMILES: Cc1nccn1CCCN(CCCCCCCC(=O)OCCC(C)CCC(C)C)CCCCCCCC(=O)OCCC(C)CCC(C)C] |
| 103 | 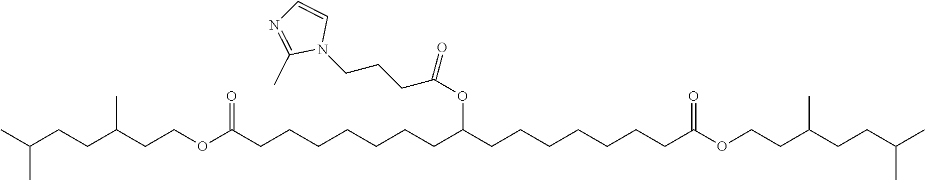 [SMILES: Cc1nccn1CCCC(=O)OC(CCCCCCCC(=O)OCCC(C)CCC(C)C)CCCCCCCC(=O)OCCC(C)CCC(C)C] |
| 104 | 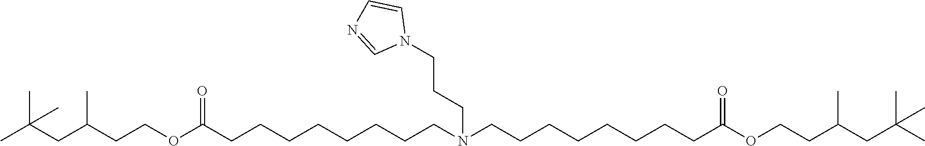 [SMILES: CC(CCOC(=O)CCCCCCCCN(CCCCCCCCC(=O)OCCC(C)CC(C)(C)C)CCCn1ccnc1)CC(C)(C)C] |
| 105 | 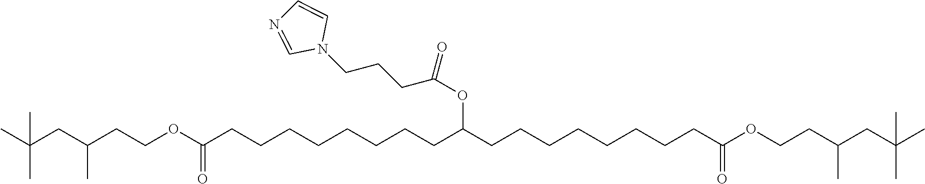 [SMILES: CC(CCOC(=O)CCCCCCCCC(CCCCCCCCC(=O)OCCC(C)CC(C)(C)C)OC(=O)CCCn1ccnc1)CC(C)(C)C] |
| 106 | 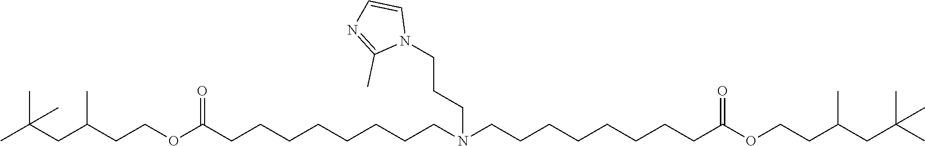 [SMILES: Cc1nccn1CCCN(CCCCCCCCC(=O)OCCC(C)CC(C)(C)C)CCCCCCCCC(=O)OCCC(C)CC(C)(C)C] |
| 107 | 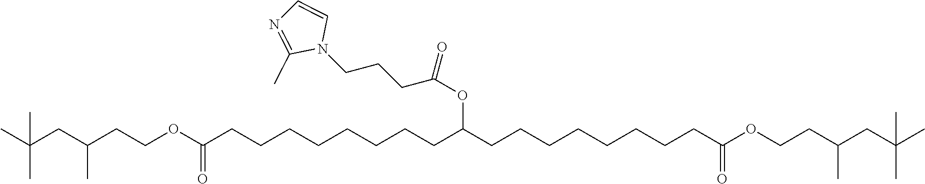 [SMILES: Cc1nccn1CCCC(=O)OC(CCCCCCCCC(=O)OCCC(C)CC(C)(C)C)CCCCCCCCC(=O)OCCC(C)CC(C)(C)C] |
| 108 | 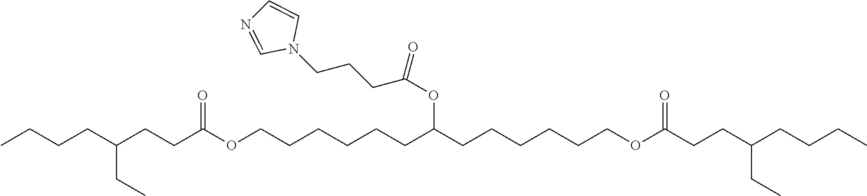 [SMILES: CCCCC(CC)CCC(=O)OCCCCCCC(CCCCCCOC(=O)CCC(CC)CCCC)OC(=O)CCCn1ccnc1] |
| 109 | 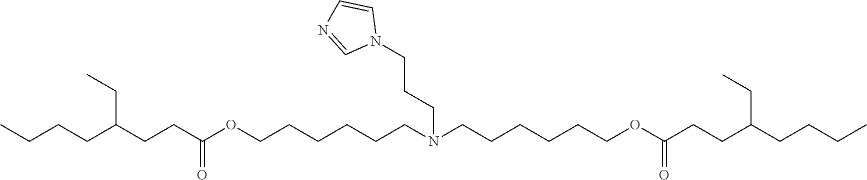 [SMILES: CCCCC(CC)CCC(=O)OCCCCCCN(CCCCCCOC(=O)CCC(CC)CCCC)CCCn1ccnc1] |
| 110 | 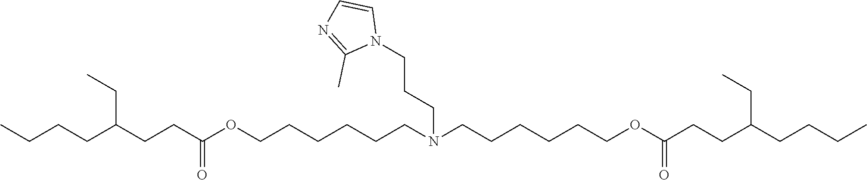 [SMILES: CCCCC(CC)CCC(=O)OCCCCCCN(CCCCCCOC(=O)CCC(CC)CCCC)CCCn1ccnc1C] |
| 111 | 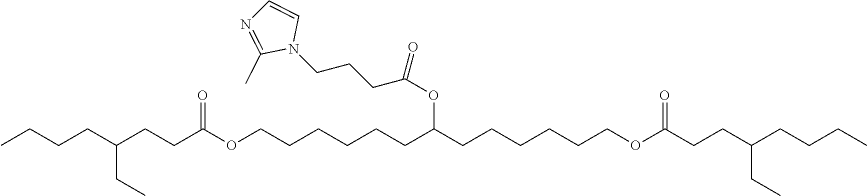 [SMILES: CCCCC(CC)CCC(=O)OCCCCCCC(CCCCCCOC(=O)CCC(CC)CCCC)OC(=O)CCCn1ccnc1C] |
| 112 | 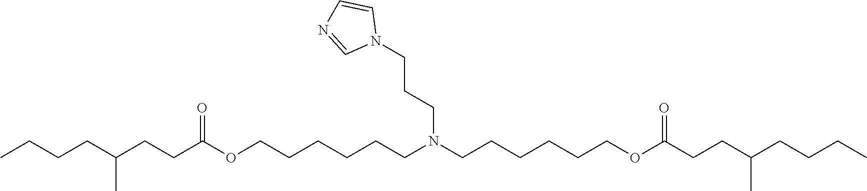 [SMILES: CCCCC(C)CCC(=O)OCCCCCCN(CCCCCCOC(=O)CCC(C)CCCC)CCCn1ccnc1] |
| 113 | 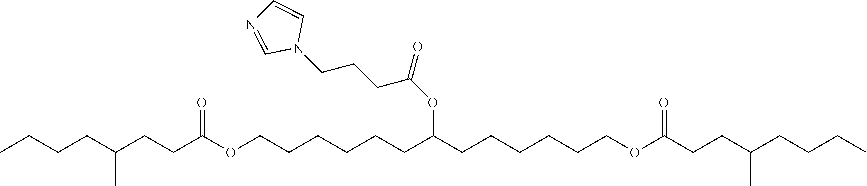 [SMILES: CCCCC(C)CCC(=O)OCCCCCCC(CCCCCCOC(=O)CCC(C)CCCC)OC(=O)CCCn1ccnc1] |
| 114 | 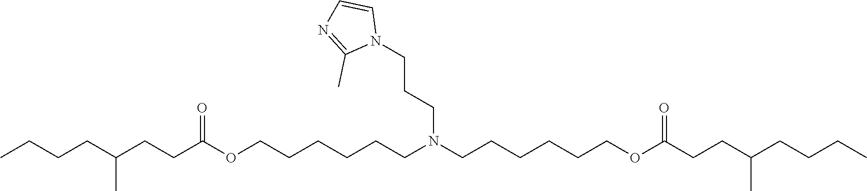 [SMILES: CCCCC(C)CCC(=O)OCCCCCCN(CCCCCCOC(=O)CCC(C)CCCC)CCCn1ccnc1C] |
| 115 | 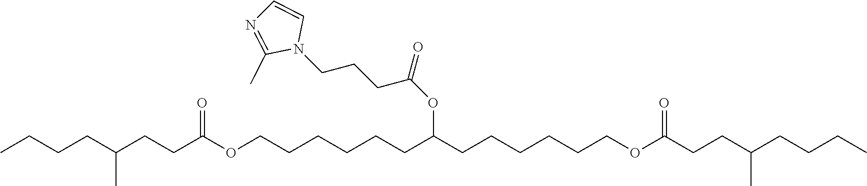 [SMILES: CCCCC(C)CCC(=O)OCCCCCCC(CCCCCCOC(=O)CCC(C)CCCC)OC(=O)CCCn1ccnc1C] |
| 116 | 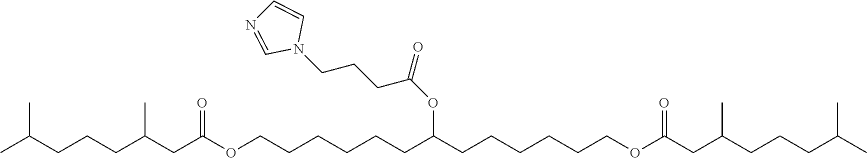 [SMILES: CC(C)CCCC(C)CC(=O)OCCCCCCC(CCCCCCOC(=O)CC(C)CCCC(C)C)OC(=O)CCCn1ccnc1] |
| 117 | 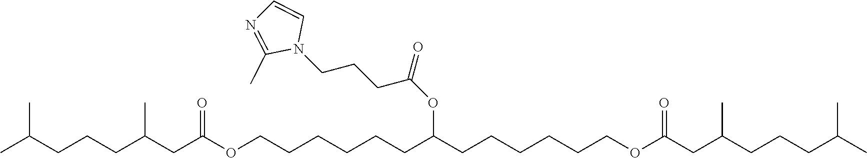 [SMILES: Cc1nccn1CCCC(=O)OC(CCCCCCOC(=O)CC(C)CCCC(C)C)CCCCCCOC(=O)CC(C)CCCC(C)C] |
| 118 | 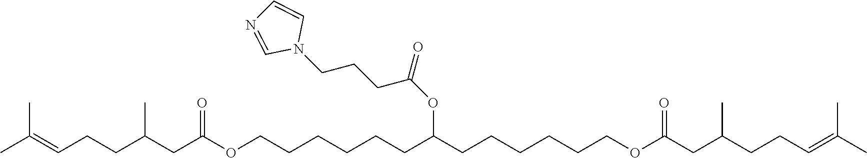 [SMILES: CC(C)=CCCC(C)CC(=O)OCCCCCCC(CCCCCCOC(=O)CC(C)CCC=C(C)C)OC(=O)CCCn1ccnc1] |
| 119 | 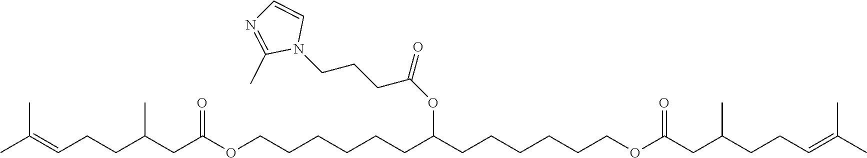 [SMILES: CC(C)=CCCC(C)CC(=O)OCCCCCCC(CCCCCCOC(=O)CC(C)CCC=C(C)C)OC(=O)CCCn1ccnc1C] |
| 120 | 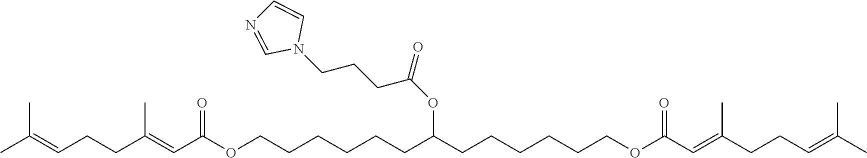 [SMILES: CC(C)=CCC/C(C)=C/C(=O)OCCCCCCC(CCCCCCOC(=O)/C=C(\C)CCC=C(C)C)OC(=O)CCCn1ccnc1] |
| 121 | 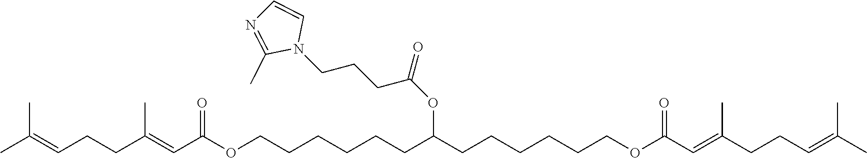 [SMILES: CC(C)=CCC/C(C)=C/C(=O)OCCCCCCC(CCCCCCOC(=O)/C=C(\C)CCC=C(C)C)OC(=O)CCCn1ccnc1C] |
| 122 | 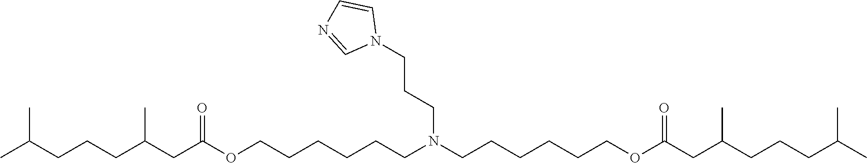 [SMILES: CC(C)CCCC(C)CC(=O)OCCCCCCN(CCCCCCOC(=O)CC(C)CCCC(C)C)CCCn1ccnc1] |
| 123 | 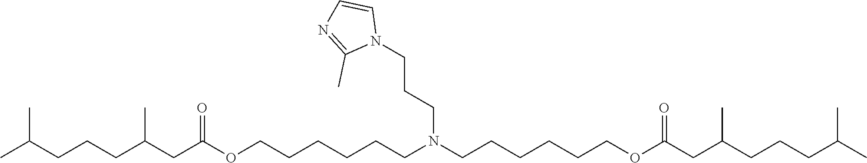 [SMILES: Cc1nccn1CCCN(CCCCCCOC(=O)CC(C)CCCC(C)C)CCCCCCOC(=O)CC(C)CCCC(C)C] |
| 124 | 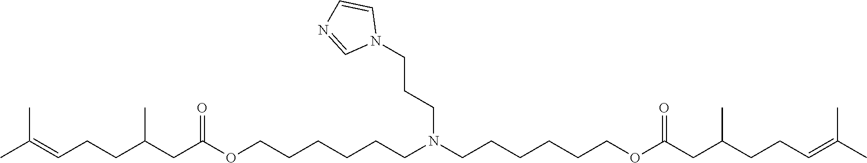 [SMILES: CC(C)=CCCC(C)CC(=O)OCCCCCCN(CCCCCCOC(=O)CC(C)CCC=C(C)C)CCCn1ccnc1] |
| 125 | 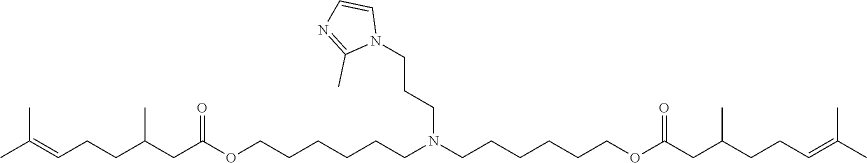 [SMILES: CC(C)=CCCC(C)CC(=O)OCCCCCCN(CCCCCCOC(=O)CC(C)CCC=C(C)C)CCCn1ccnc1C] |
| 126 | 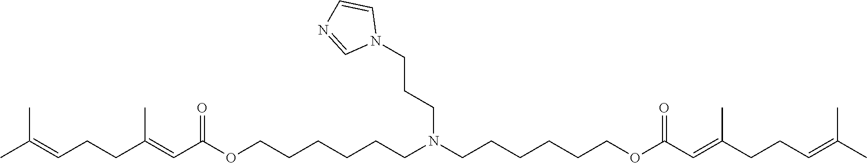 [SMILES: CC(C)=CCC/C(C)=C/C(=O)OCCCCCCN(CCCCCCOC(=O)/C=C(\C)CCC=C(C)C)CCCn1ccnc1] |
| 127 | 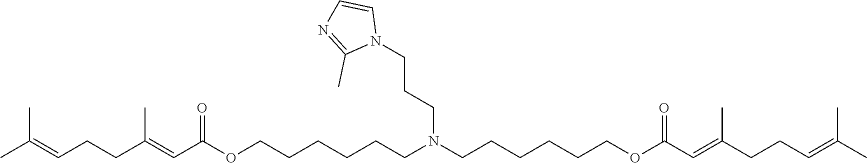 [SMILES: CC(C)=CCC/C(C)=C/C(=O)OCCCCCCN(CCCCCCOC(=O)/C=C(\C)CCC=C(C)C)CCCn1ccnc1C] |
| 128 | 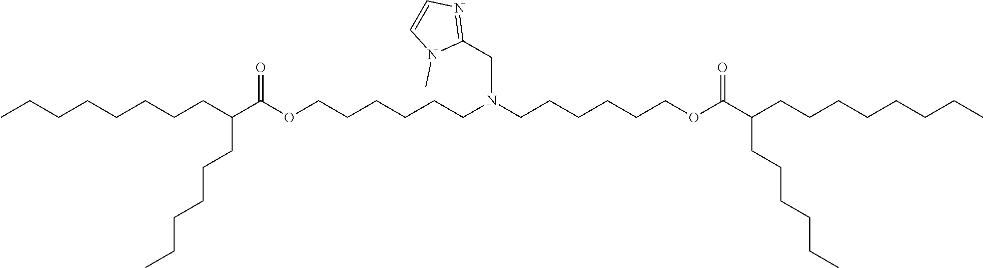 [SMILES: CCCCCCCCC(CCCCCC)C(=O)OCCCCCCN(CCCCCCOC(=O)C(CCCCCC)CCCCCCCC)Cc1nccn1C] |
| 129 | 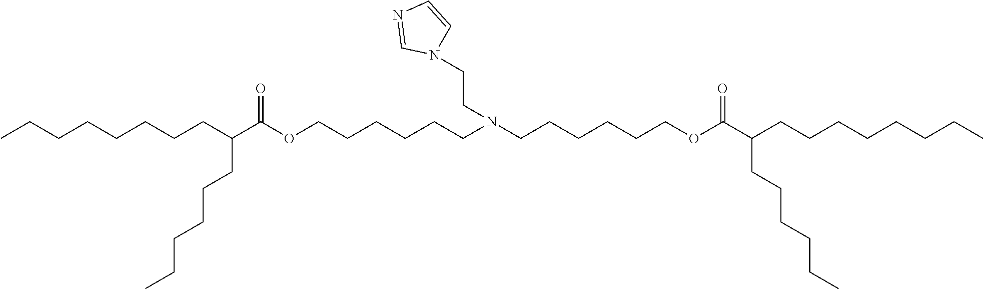 [SMILES: CCCCCCCCC(CCCCCC)C(=O)OCCCCCCN(CCCCCCOC(=O)C(CCCCCC)CCCCCCCC)CCn1ccnc1] |
| 130 | 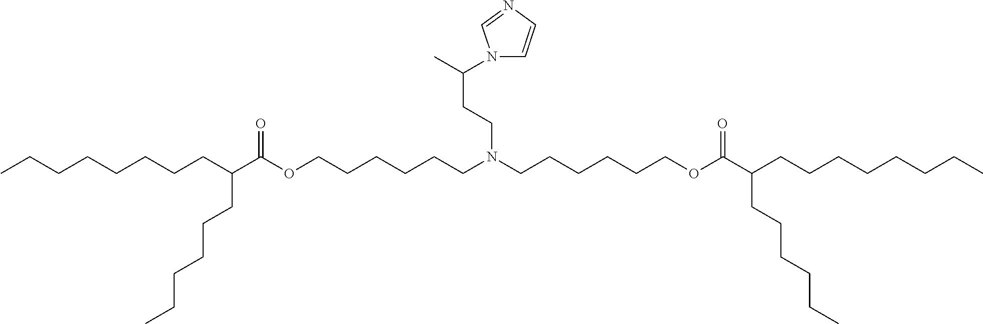 [SMILES: CCCCCCCCC(CCCCCC)C(=O)OCCCCCCN(CCCCCCOC(=O)C(CCCCCC)CCCCCCCC)CCC(C)n1ccnc1] |
| 131 | 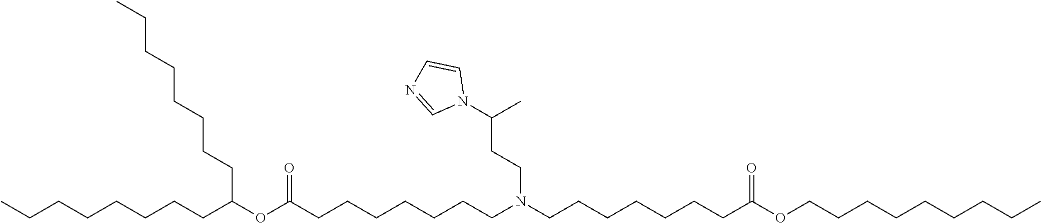 [SMILES: CCCCCCCCCOC(=O)CCCCCCCN(CCCCCCCC(=O)OC(CCCCCCCC)CCCCCCCC)CCC(C)n1ccnc1] |
| 132 | 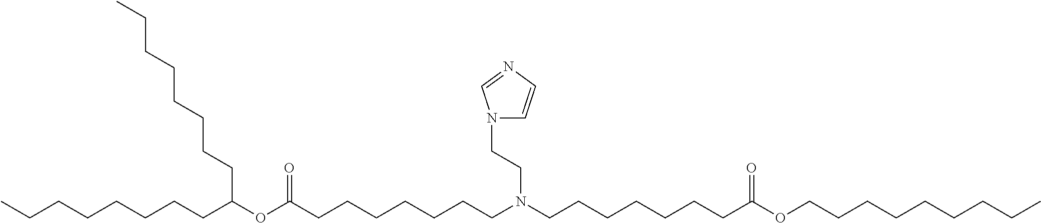 [SMILES: CCCCCCCCCOC(=O)CCCCCCCN(CCCCCCCC(=O)OC(CCCCCCCC)CCCCCCCC)CCn1ccnc1] |
| 133 | 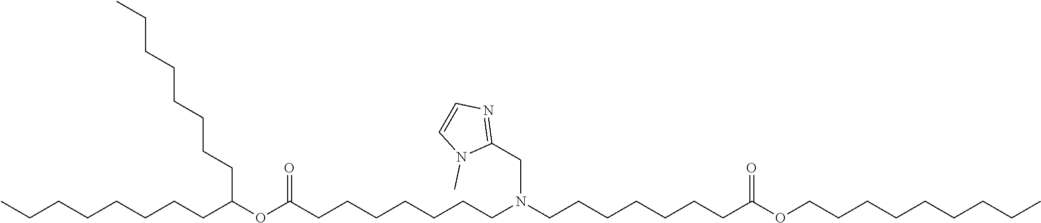 [SMILES: CCCCCCCCCOC(=O)CCCCCCCN(CCCCCCCC(=O)OC(CCCCCCCC)CCCCCCCC)Cc1nccn1C] |
| 134 | 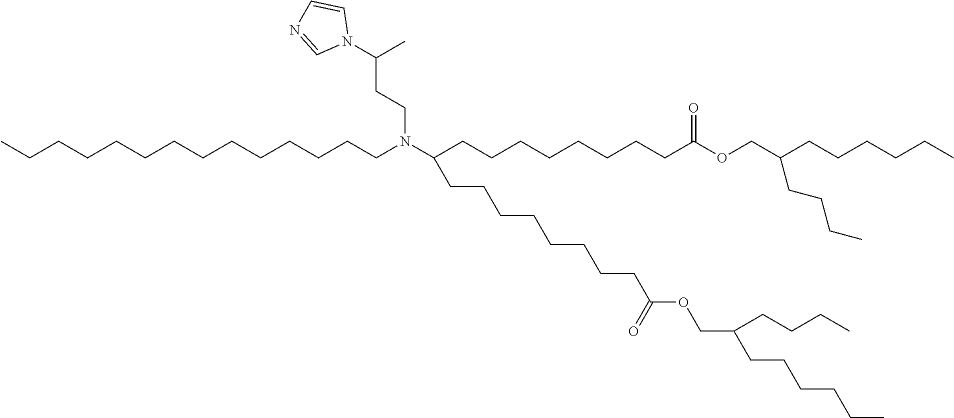 [SMILES: CCCCCCCCCCCCCCN(CCC(C)n1ccnc1)C(CCCCCCCCC(=O)OCC(CCCC)CCCCCC)CCCCCCCCC(=O)OCC(CCCC)CCCCCC] |
| 135 | 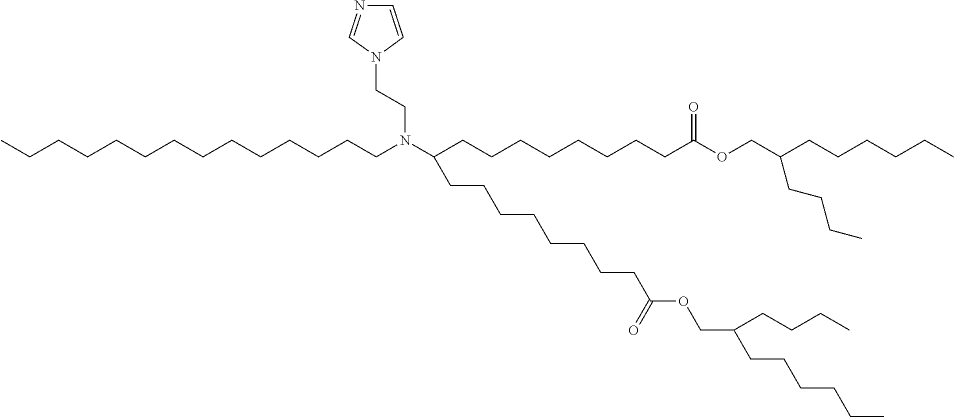 [SMILES: CCCCCCCCCCCCCCN(CCn1ccnc1)C(CCCCCCCCC(=O)OCC(CCCC)CCCCCC)CCCCCCCCC(=O)OCC(CCCC)CCCCCC] |
| 136 | 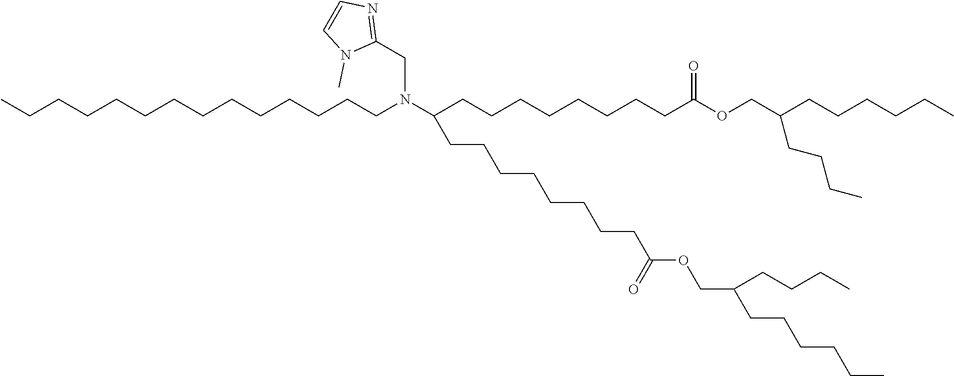 [SMILES: CCCCCCCCCCCCCCN(Cc1nccn1C)C(CCCCCCCCC(=O)OCC(CCCC)CCCCCC)CCCCCCCCC(=O)OCC(CCCC)CCCCCC] |
| 137 | 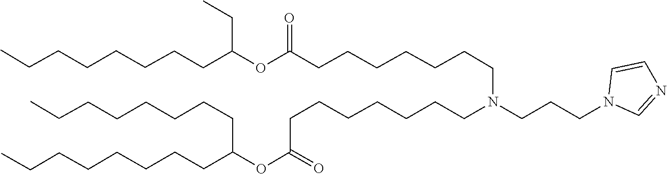 [SMILES: CCCCCCCCC(CC)OC(=O)CCCCCCCN(CCCCCCCC(=O)OC(CCCCCCCC)CCCCCCCC)CCCn1ccnc1] |
| 138 | 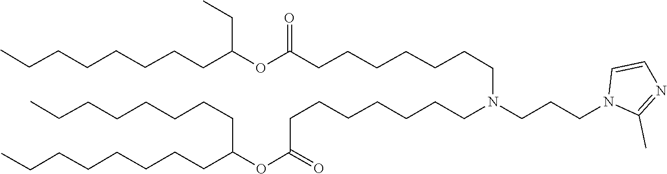 [SMILES: CCCCCCCCC(CC)OC(=O)CCCCCCCN(CCCCCCCC(=O)OC(CCCCCCCC)CCCCCCCC)CCCn1ccnc1C] |
| 139 | 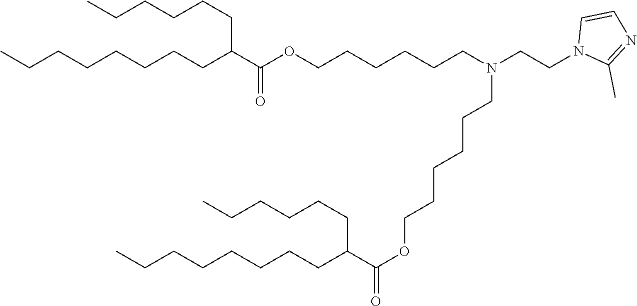 [SMILES: CCCCCCCCC(CCCCCC)C(=O)OCCCCCCN(CCCCCCOC(=O)C(CCCCCC)CCCCCCCC)CCn1ccnc1C] |
| 140 | 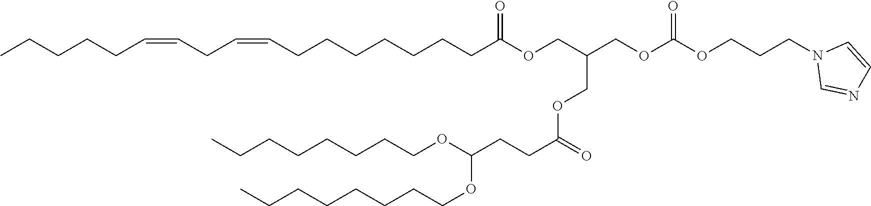 [SMILES: CCCCC/C=C\C/C=C\CCCCCCCC(=O)OCC(COC(=O)CCC(OCCCCCCCC)OCCCCCCCC)COC(=O)OCCCn1ccnc1] |
| 141 | 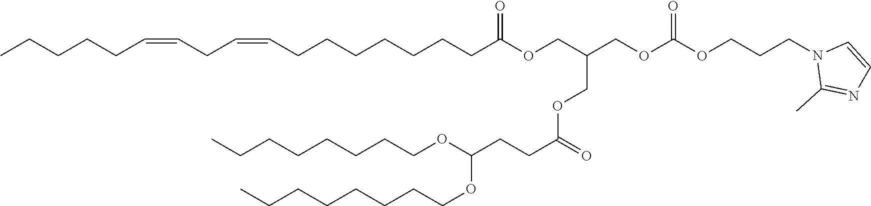 [SMILES: CCCCC/C=C\C/C=C\CCCCCCCC(=O)OCC(COC(=O)CCC(OCCCCCCCC)OCCCCCCCC)COC(=O)OCCCn1ccnc1C] |
| 142 | 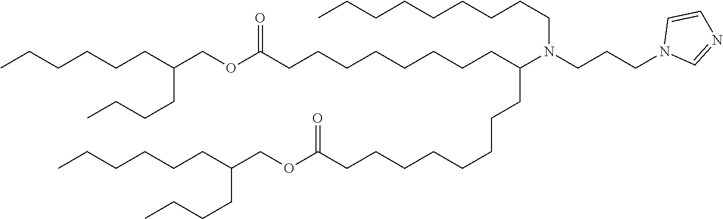 [SMILES: CCCCCCCCCN(CCCn1ccnc1)C(CCCCCCCCC(=O)OCC(CCCC)CCCCCC)CCCCCCCCC(=O)OCC(CCCC)CCCCCC] |
| 143 | 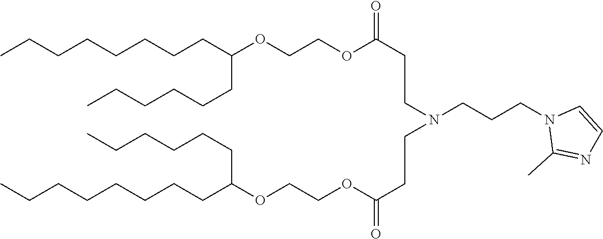 [SMILES: CCCCCCCCC(CCCCCC)OCCOC(=O)CCN(CCCn1ccnc1C)CCC(=O)OCCOC(CCCCCC)CCCCCCCC] |
| 144 | 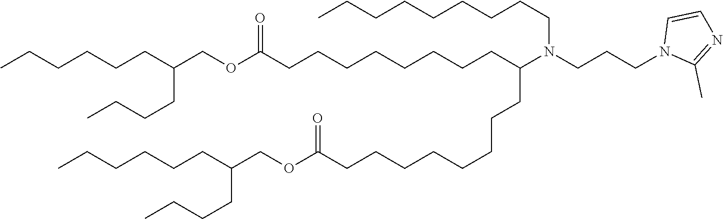 [SMILES: CCCCCCCCCN(CCCn1ccnc1C)C(CCCCCCCCC(=O)OCC(CCCC)CCCCCC)CCCCCCCCC(=O)OCC(CCCC)CCCCCC] |
| 145 | 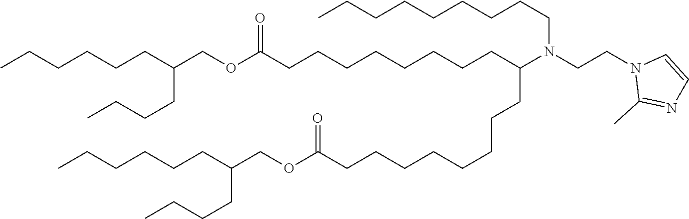 [SMILES: CCCCCCCCCN(CCn1ccnc1C)C(CCCCCCCCC(=O)OCC(CCCC)CCCCCC)CCCCCCCCC(=O)OCC(CCCC)CCCCCC] |
| 146 | 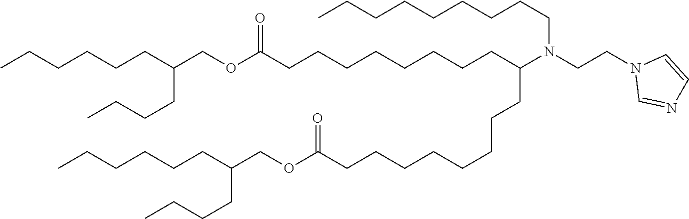 [SMILES: CCCCCCCCCN(CCn1ccnc1)C(CCCCCCCCC(=O)OCC(CCCC)CCCCCC)CCCCCCCCC(=O)OCC(CCCC)CCCCCC] |
| 147 | 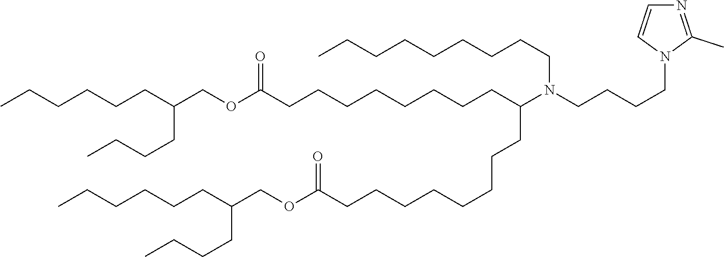 [SMILES: CCCCCCCCCN(CCCCn1ccnc1C)C(CCCCCCCCC(=O)OCC(CCCC)CCCCCC)CCCCCCCCC(=O)OCC(CCCC)CCCCCC] |
| 148 | 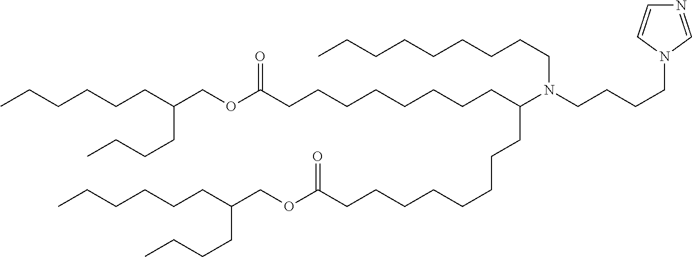 [SMILES: CCCCCCCCCN(CCCCn1ccnc1)C(CCCCCCCCC(=O)OCC(CCCC)CCCCCC)CCCCCCCCC(=O)OCC(CCCC)CCCCCC] |

In some embodiments, the ionizable lipid has a beta-hydroxyl amine head group. In some embodiments, the ionizable lipid has a gamma-hydroxyl amine head group.

In some embodiments, an ionizable lipid of the disclosure is a lipid selected from Table 10b. In some embodiments, an ionizable lipid of the disclosure is Lipid 15 from Table 10b. In an embodiment, the ionizable lipid is described in US patent publication number US20170210697A1. In an embodiment, the ionizable lipid is described in US patent publication number US20170119904A1.

TABLE 10b

| Ionizable | |
|---|---|
| lipid | |
| number | Structure |
| 1 | 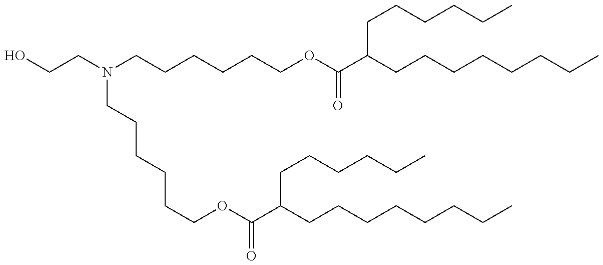 [SMILES: CCCCCCCCC(CCCCCC)C(=O)OCCCCCCN(CCO)CCCCCCOC(=O)C(CCCCCC)CCCCCCCC] |
| 2 | 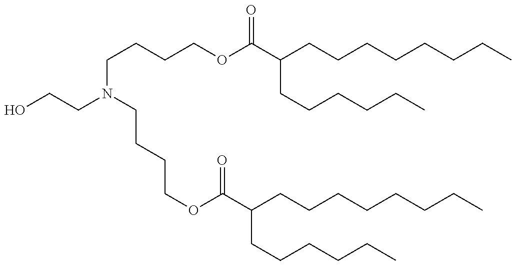 [SMILES: CCCCCCCCC(CCCCCC)C(=O)OCCCCN(CCO)CCCCOC(=O)C(CCCCCC)CCCCCCCC] |
| 3 | 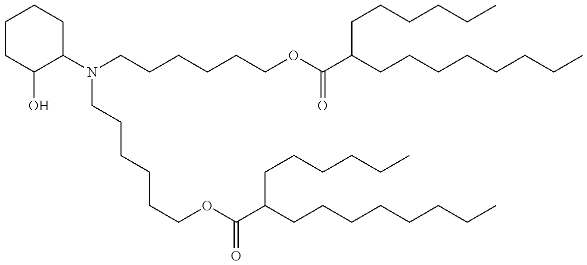 [SMILES: CCCCCCCCC(CCCCCC)C(=O)OCCCCCCN(CCCCCCOC(=O)C(CCCCCC)CCCCCCCC)C1CCCCC1O] |
| 4 | 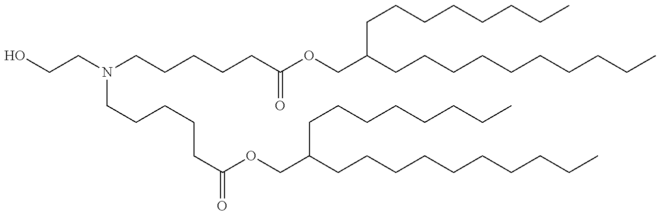 [SMILES: CCCCCCCCCCC(CCCCCCCC)COC(=O)CCCCCN(CCO)CCCCCC(=O)OCC(CCCCCCCC)CCCCCCCCCC] |
| 5 | 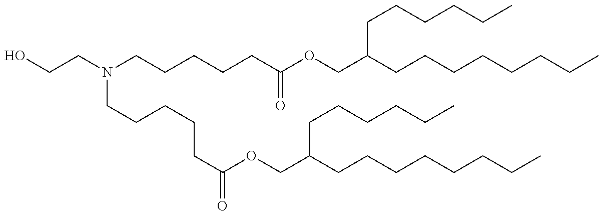 [SMILES: CCCCCCCCC(CCCCCC)COC(=O)CCCCCN(CCO)CCCCCC(=O)OCC(CCCCCC)CCCCCCCC] |
| 6 | 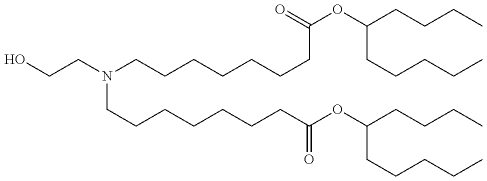 [SMILES: CCCCCC(CCCC)OC(=O)CCCCCCCN(CCO)CCCCCCCC(=O)OC(CCCC)CCCCC] |
| 7 | 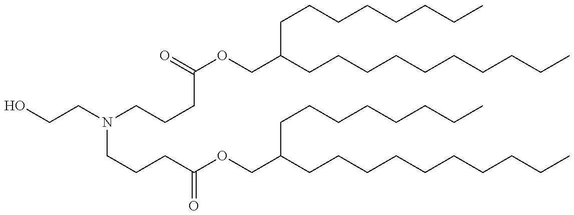 [SMILES: CCCCCCCCCCC(CCCCCCCC)COC(=O)CCCN(CCO)CCCC(=O)OCC(CCCCCCCC)CCCCCCCCCC] |
| 8 | 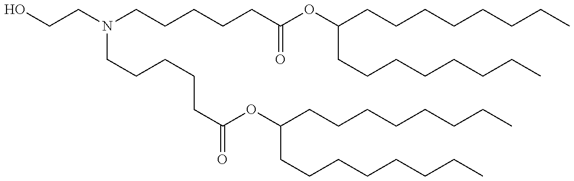 [SMILES: CCCCCCCCC(CCCCCCCC)OC(=O)CCCCCN(CCO)CCCCCC(=O)OC(CCCCCCCC)CCCCCCCC] |
| 9 | 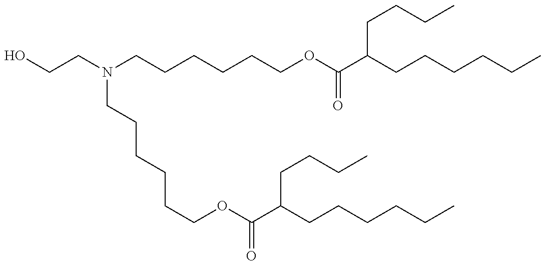 [SMILES: CCCCCCC(CCCC)C(=O)OCCCCCCN(CCO)CCCCCCOC(=O)C(CCCC)CCCCCC] |
| 10 | 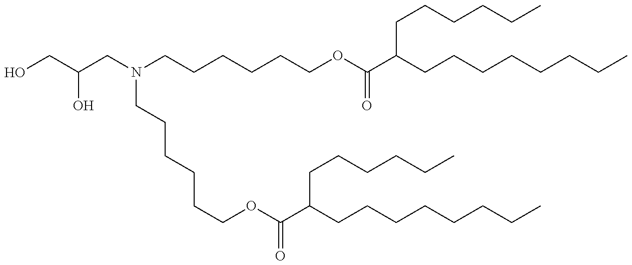 [SMILES: CCCCCCCCC(CCCCCC)C(=O)OCCCCCCN(CCCCCCOC(=O)C(CCCCCC)CCCCCCCC)CC(O)CO] |
| 11 | 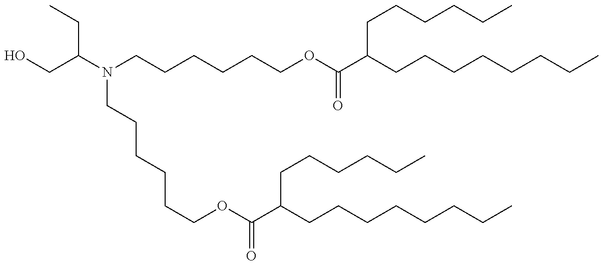 [SMILES: CCCCCCCCC(CCCCCC)C(=O)OCCCCCCN(CCCCCCOC(=O)C(CCCCCC)CCCCCCCC)C(CC)CO] |
| 12 | 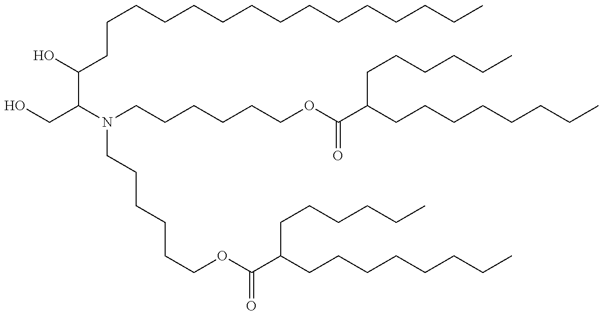 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| 13 | 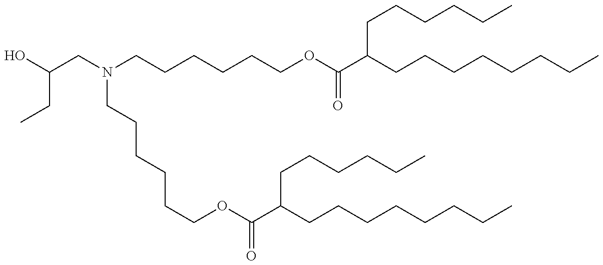 [SMILES: CCCCCCCCC(CCCCCC)C(=O)OCCCCCCN(CCCCCCOC(=O)C(CCCCCC)CCCCCCCC)CC(O)CC] |
| 14 | 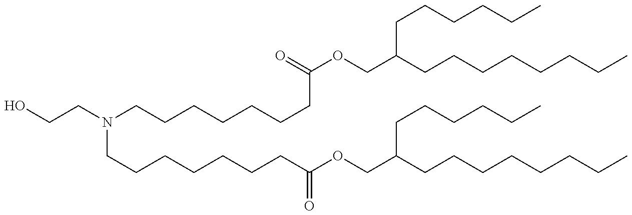 [SMILES: CCCCCCCCC(CCCCCC)COC(=O)CCCCCCCN(CCO)CCCCCCCC(=O)OCC(CCCCCC)CCCCCCCC] |
| 15 | 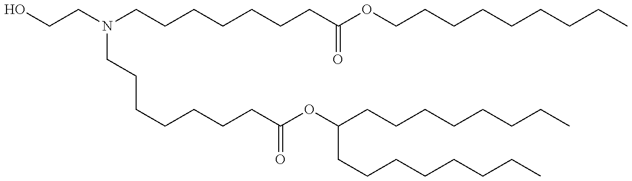 [SMILES: CCCCCCCCCOC(=O)CCCCCCCN(CCO)CCCCCCCC(=O)OC(CCCCCCCC)CCCCCCCC] |
| 16 | 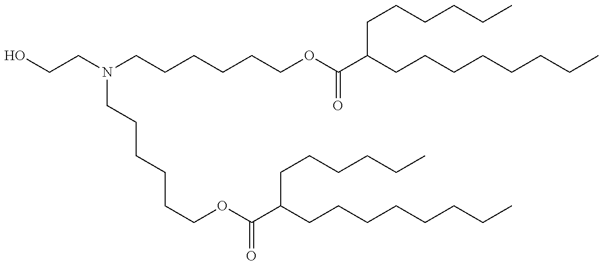 [SMILES: CCCCCCCCC(CCCCCC)C(=O)OCCCCCCN(CCO)CCCCCCOC(=O)C(CCCCCC)CCCCCCCC] |
| 17 | 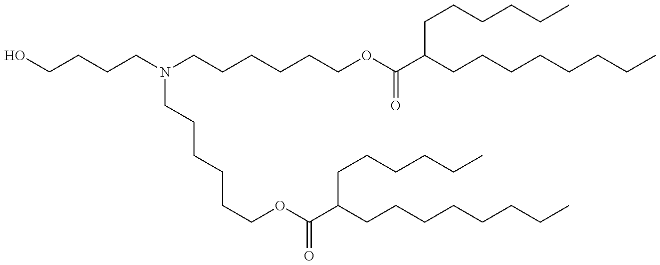 [SMILES: CCCCCCCCC(CCCCCC)C(=O)OCCCCCCN(CCCCO)CCCCCCOC(=O)C(CCCCCC)CCCCCCCC] |
| 18 | 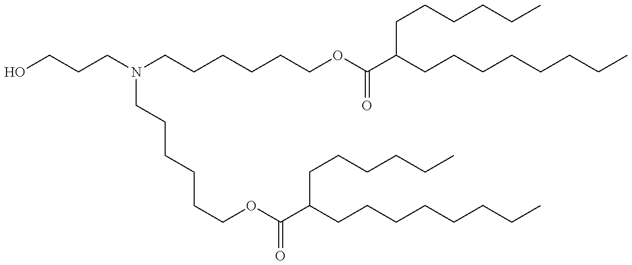 [SMILES: CCCCCCCCC(CCCCCC)C(=O)OCCCCCCN(CCCO)CCCCCCOC(=O)C(CCCCCC)CCCCCCCC] |
| 19 | 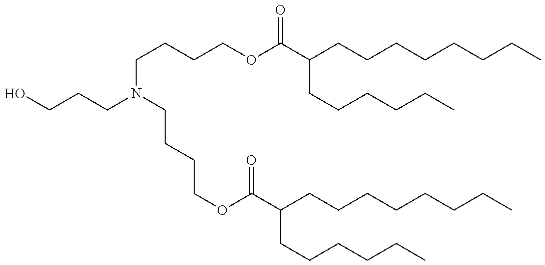 [SMILES: CCCCCCCCC(CCCCCC)C(=O)OCCCCN(CCCO)CCCCOC(=O)C(CCCCCC)CCCCCCCC] |
| 20 | 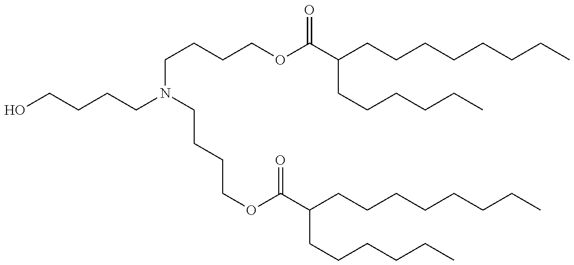 [SMILES: CCCCCCCCC(CCCCCC)C(=O)OCCCCN(CCCCO)CCCCOC(=O)C(CCCCCC)CCCCCCCC] |
| 21 | 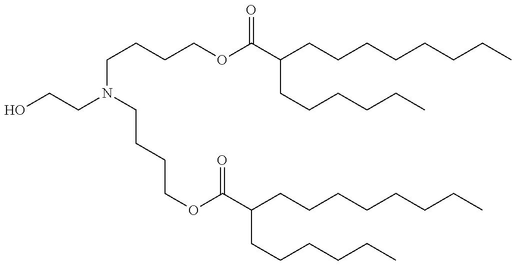 [SMILES: CCCCCCCCC(CCCCCC)C(=O)OCCCCN(CCO)CCCCOC(=O)C(CCCCCC)CCCCCCCC] |
| 22 | 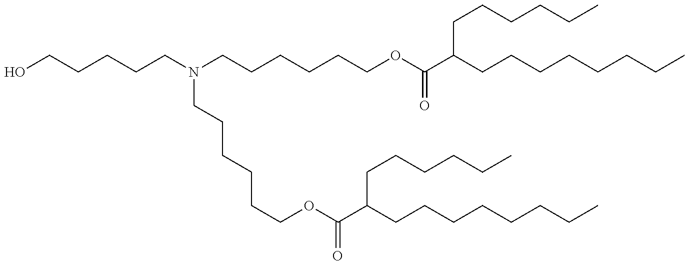 [SMILES: CCCCCCCCC(CCCCCC)C(=O)OCCCCCCN(CCCCCO)CCCCCCOC(=O)C(CCCCCC)CCCCCCCC] |
| 23 | 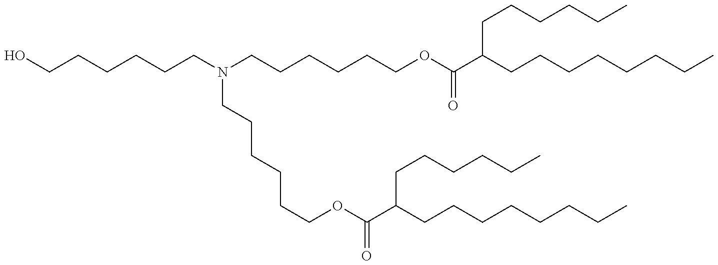 [SMILES: CCCCCCCCC(CCCCCC)C(=O)OCCCCCCN(CCCCCCO)CCCCCCOC(=O)C(CCCCCC)CCCCCCCC] |
| 24 | 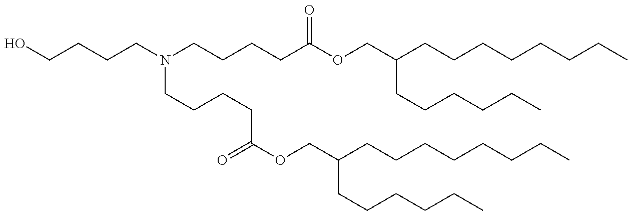 [SMILES: CCCCCCCCC(CCCCCC)COC(=O)CCCCN(CCCCO)CCCCC(=O)OCC(CCCCCC)CCCCCCCC] |
| 25 | 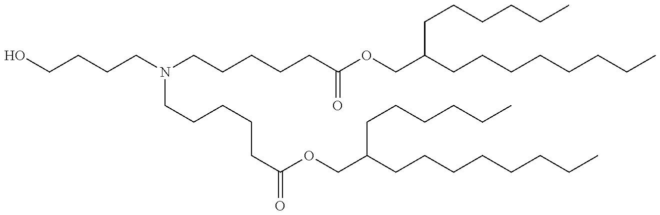 [SMILES: CCCCCCCCC(CCCCCC)COC(=O)CCCCCN(CCCCO)CCCCCC(=O)OCC(CCCCCC)CCCCCCCC] |
| 26 | 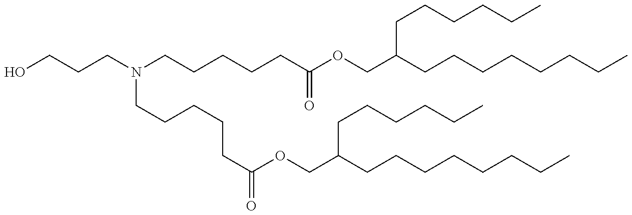 [SMILES: CCCCCCCCC(CCCCCC)COC(=O)CCCCCN(CCCO)CCCCCC(=O)OCC(CCCCCC)CCCCCCCC] |
| 27 | 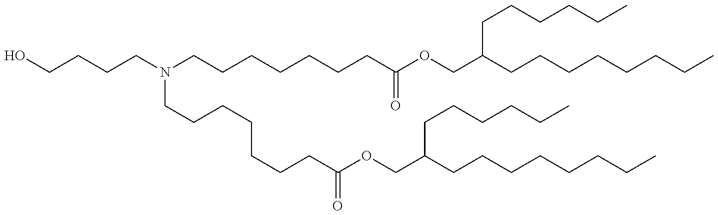 [SMILES: CCCCCCCCC(CCCCCC)COC(=O)CCCCCCCN(CCCCO)CCCCCCCC(=O)OCC(CCCCCC)CCCCCCCC] |
| 28 | 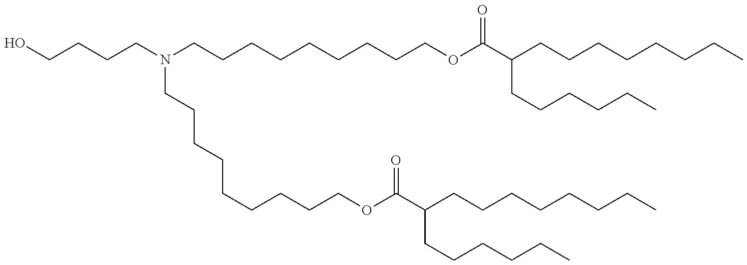 [SMILES: CCCCCCCCC(CCCCCC)C(=O)OCCCCCCCCCN(CCCCO)CCCCCCCCCOC(=O)C(CCCCCC)CCCCCCCC] |
| 29 | 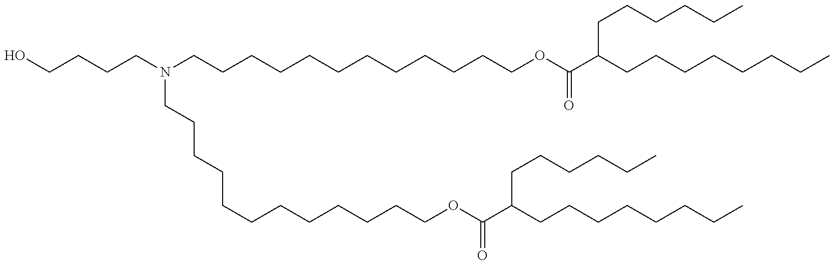 [SMILES: CCCCCCCCC(CCCCCC)C(=O)OCCCCCCCCCCCCN(CCCCO)CCCCCCCCCCCCOC(=O)C(CCCCCC)CCCCCCCC] |
| 30 | 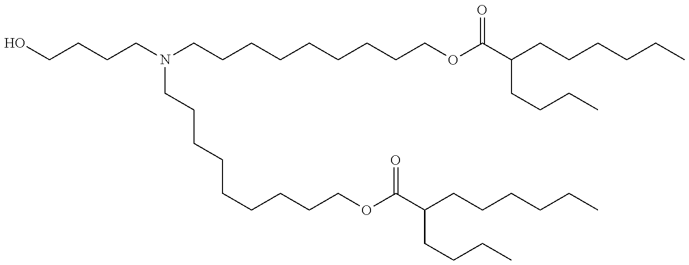 [SMILES: CCCCCCC(CCCC)C(=O)OCCCCCCCCCN(CCCCO)CCCCCCCCCOC(=O)C(CCCC)CCCCCC] |
| 31 | 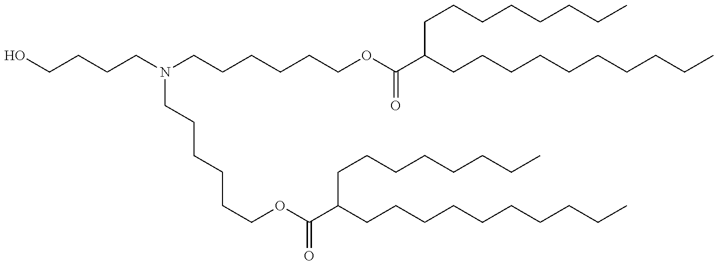 [SMILES: CCCCCCCCCCC(CCCCCCCC)C(=O)OCCCCCCN(CCCCO)CCCCCCOC(=O)C(CCCCCCCC)CCCCCCCCCC] |
| 32 | 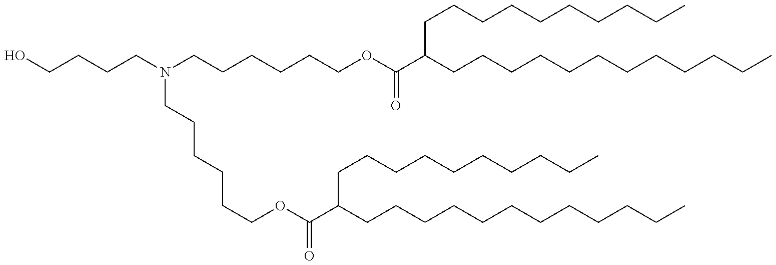 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| 33 | 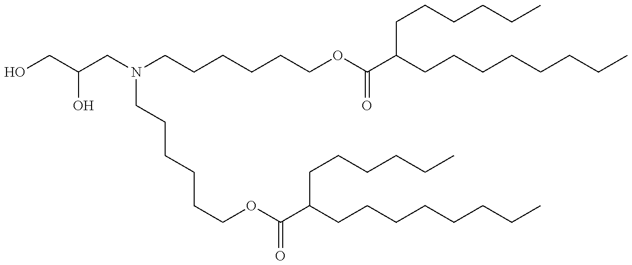 [SMILES: CCCCCCCCC(CCCCCC)C(=O)OCCCCCCN(CCCCCCOC(=O)C(CCCCCC)CCCCCCCC)CC(O)CO] |
| 34 | 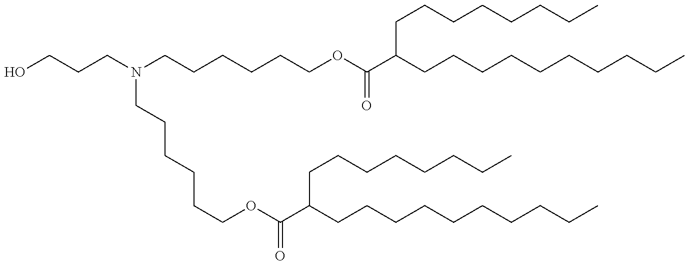 [SMILES: CCCCCCCCCCC(CCCCCCCC)C(=O)OCCCCCCN(CCCO)CCCCCCOC(=O)C(CCCCCCCC)CCCCCCCCCC] |
| 35 | 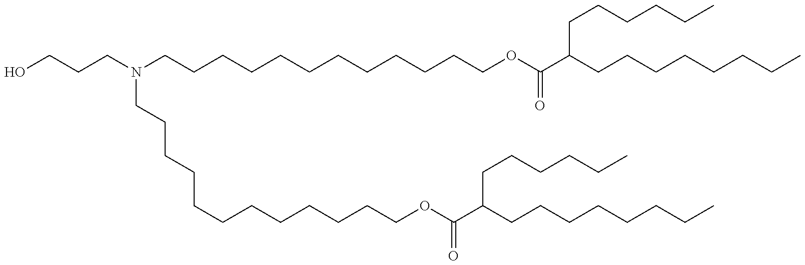 [SMILES: CCCCCCCCC(CCCCCC)C(=O)OCCCCCCCCCCCCN(CCCO)CCCCCCCCCCCCOC(=O)C(CCCCCC)CCCCCCCC] |
| 36 | 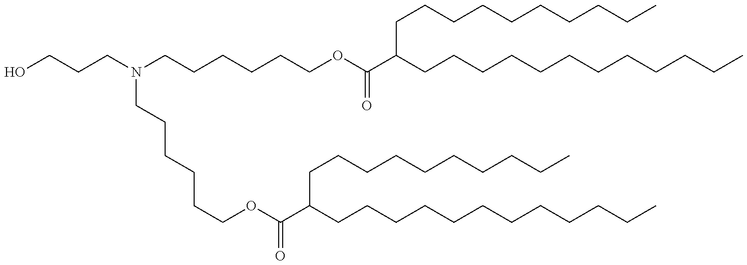 [SMILES: CCCCCCCCCCCCC(CCCCCCCCCC)C(=O)OCCCCCCN(CCCO)CCCCCCOC(=O)C(CCCCCCCCCC)CCCCCCCCCCCC] |
| 37 | 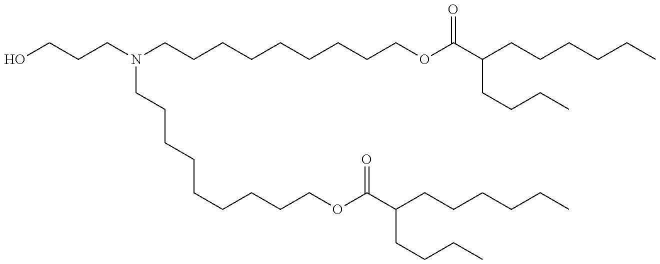 [SMILES: CCCCCCC(CCCC)C(=O)OCCCCCCCCCN(CCCO)CCCCCCCCCOC(=O)C(CCCC)CCCCCC] |
| 38 | 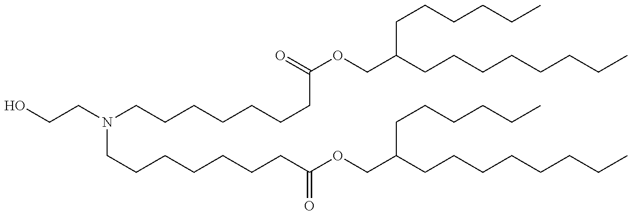 [SMILES: CCCCCCCCC(CCCCCC)COC(=O)CCCCCCCN(CCO)CCCCCCCC(=O)OCC(CCCCCC)CCCCCCCC] |

In some embodiments, an ionizable lipid has one of the structures set forth in Table 10 below.

TABLE 10

| Number | Structure |
|---|---|
| 1 | 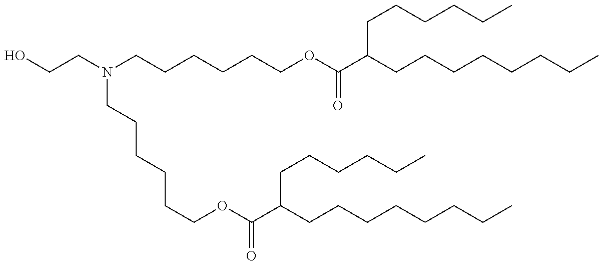 [SMILES: CCCCCCCCC(CCCCCC)C(=O)OCCCCCCN(CCO)CCCCCCOC(=O)C(CCCCCC)CCCCCCCC] |
| 2 | 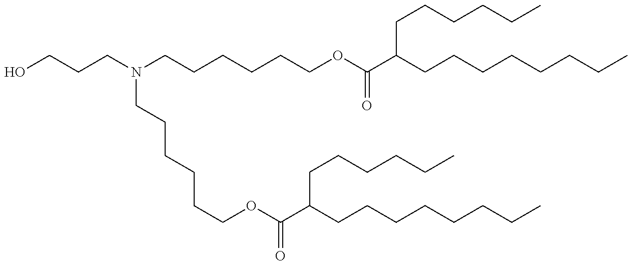 [SMILES: CCCCCCCCC(CCCCCC)C(=O)OCCCCCCN(CCCO)CCCCCCOC(=O)C(CCCCCC)CCCCCCCC] |
| 3 | 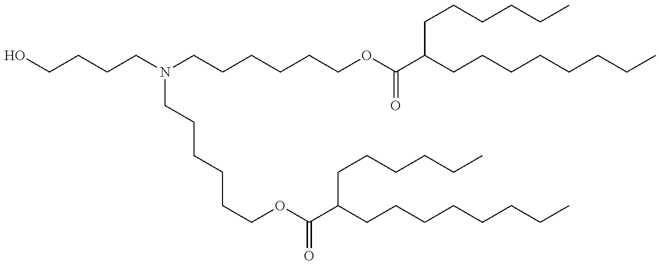 [SMILES: CCCCCCCCC(CCCCCC)C(=O)OCCCCCCN(CCCCO)CCCCCCOC(=O)C(CCCCCC)CCCCCCCC] |
| 4 | 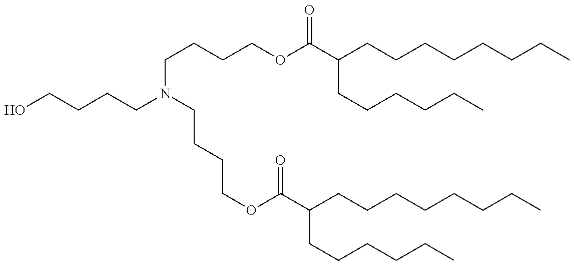 [SMILES: CCCCCCCCC(CCCCCC)C(=O)OCCCCN(CCCCO)CCCCOC(=O)C(CCCCCC)CCCCCCCC] |
| 5 | 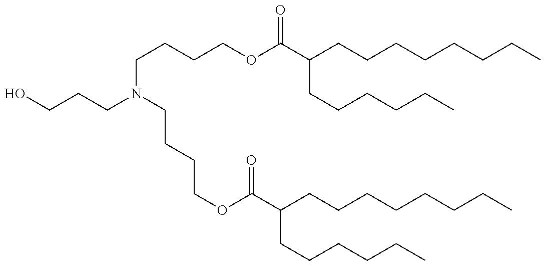 [SMILES: CCCCCCCCC(CCCCCC)C(=O)OCCCCN(CCCO)CCCCOC(=O)C(CCCCCC)CCCCCCCC] |
| 6 | 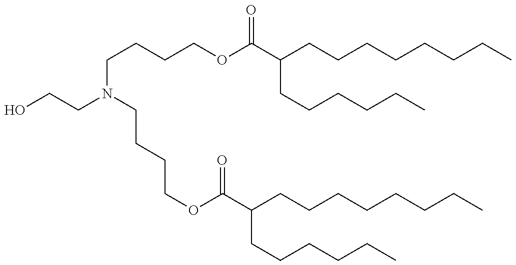 [SMILES: CCCCCCCCC(CCCCCC)C(=O)OCCCCN(CCO)CCCCOC(=O)C(CCCCCC)CCCCCCCC] |
| 7 | 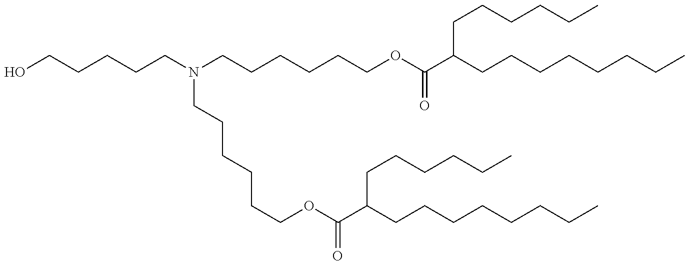 [SMILES: CCCCCCCCC(CCCCCC)C(=O)OCCCCCCN(CCCCCO)CCCCCCOC(=O)C(CCCCCC)CCCCCCCC] |
| 8 | 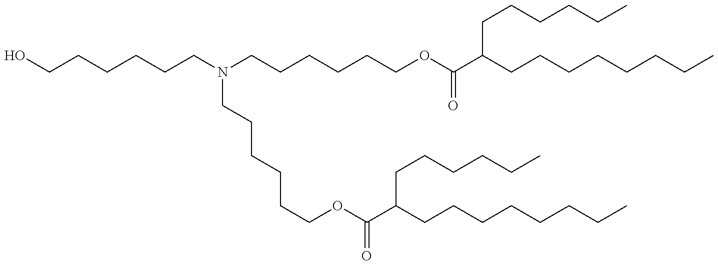 [SMILES: CCCCCCCCC(CCCCCC)C(=O)OCCCCCCN(CCCCCCO)CCCCCCOC(=O)C(CCCCCC)CCCCCCCC] |
| 9 | 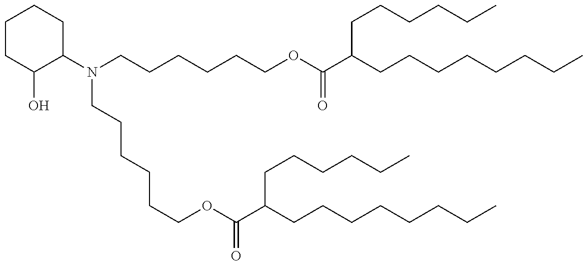 [SMILES: CCCCCCCCC(CCCCCC)C(=O)OCCCCCCN(CCCCCCOC(=O)C(CCCCCC)CCCCCCCC)C1CCCCC1O] |
| 10 | 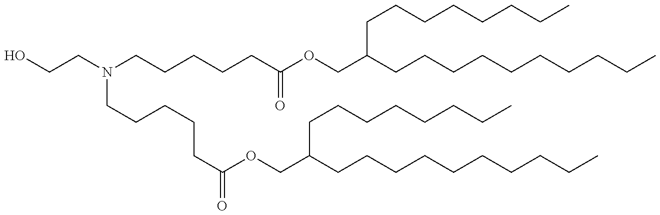 [SMILES: CCCCCCCCCCC(CCCCCCCC)COC(=O)CCCCCN(CCO)CCCCCC(=O)OCC(CCCCCCCC)CCCCCCCCCC] |
| 11 | 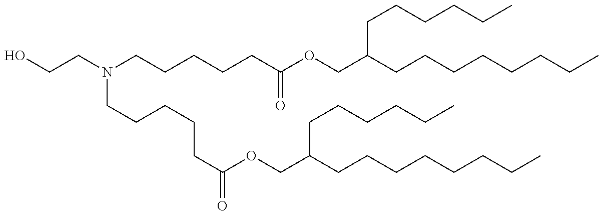 [SMILES: CCCCCCCCC(CCCCCC)COC(=O)CCCCCN(CCO)CCCCCC(=O)OCC(CCCCCC)CCCCCCCC] |
| 12 | 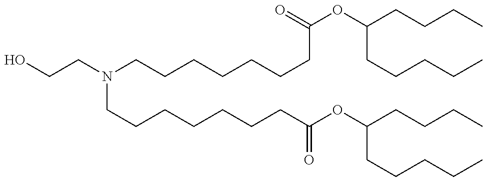 [SMILES: CCCCCC(CCCC)OC(=O)CCCCCCCN(CCO)CCCCCCCC(=O)OC(CCCC)CCCCC] |
| 13 | 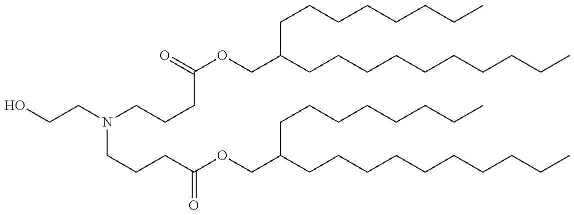 [SMILES: CCCCCCCCCCC(CCCCCCCC)COC(=O)CCCN(CCO)CCCC(=O)OCC(CCCCCCCC)CCCCCCCCCC] |
| 14 | 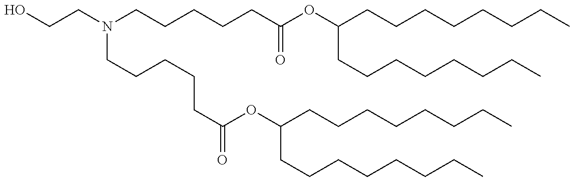 [SMILES: CCCCCCCCC(CCCCCCCC)OC(=O)CCCCCN(CCO)CCCCCC(=O)OC(CCCCCCCC)CCCCCCCC] |
| 15 | 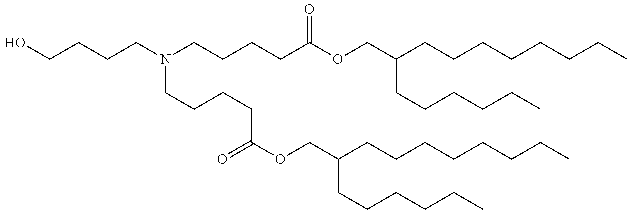 [SMILES: CCCCCCCCC(CCCCCC)COC(=O)CCCCN(CCCCO)CCCCC(=O)OCC(CCCCCC)CCCCCCCC] |
| 16 | 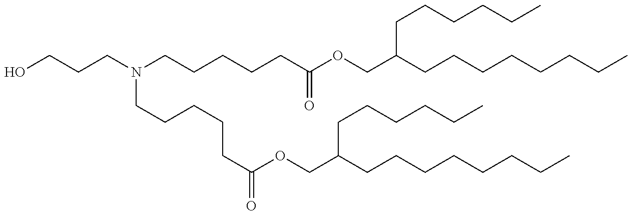 [SMILES: CCCCCCCCC(CCCCCC)COC(=O)CCCCCN(CCCO)CCCCCC(=O)OCC(CCCCCC)CCCCCCCC] |
| 17 | 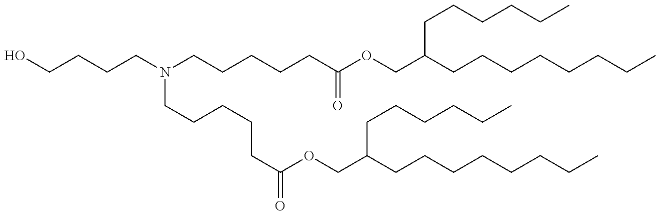 [SMILES: CCCCCCCCC(CCCCCC)COC(=O)CCCCCN(CCCCO)CCCCCC(=O)OCC(CCCCCC)CCCCCCCC] |
| 18 | 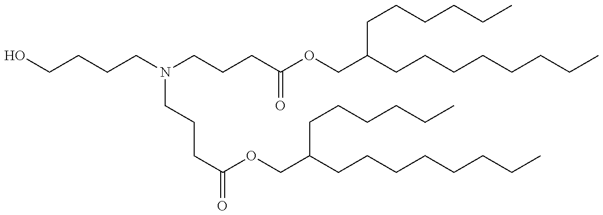 [SMILES: CCCCCCCCC(CCCCCC)COC(=O)CCCN(CCCCO)CCCC(=O)OCC(CCCCCC)CCCCCCCC] |
| 19 | 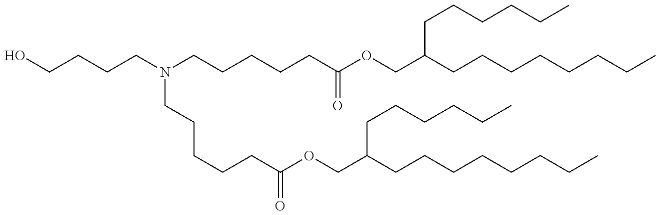 [SMILES: CCCCCCCCC(CCCCCC)COC(=O)CCCCCN(CCCCO)CCCCCC(=O)OCC(CCCCCC)CCCCCCCC] |
| 20 | 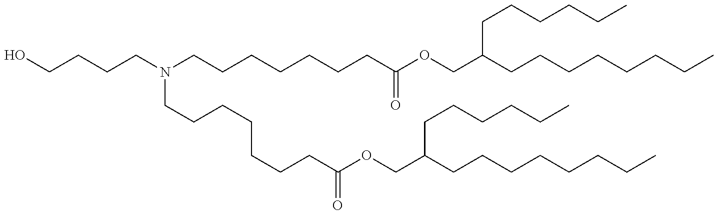 [SMILES: CCCCCCCCC(CCCCCC)COC(=O)CCCCCCCN(CCCCO)CCCCCCCC(=O)OCC(CCCCCC)CCCCCCCC] |
| 21 | 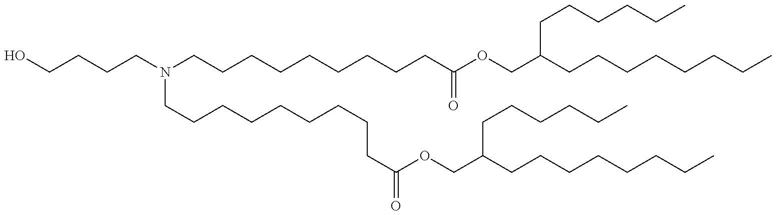 [SMILES: CCCCCCCCC(CCCCCC)COC(=O)CCCCCCCCCN(CCCCO)CCCCCCCCCC(=O)OCC(CCCCCC)CCCCCCCC] |
| 22 | 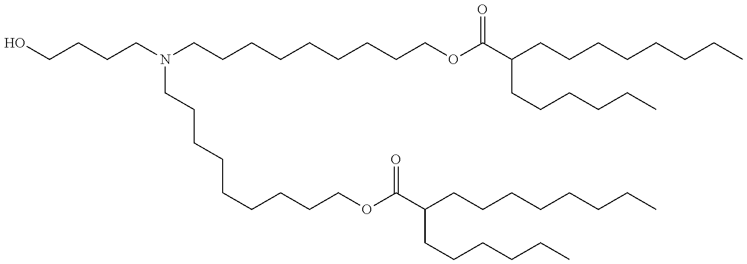 [SMILES: CCCCCCCCC(CCCCCC)C(=O)OCCCCCCCCCN(CCCCO)CCCCCCCCCOC(=O)C(CCCCCC)CCCCCCCC] |
| 23 | 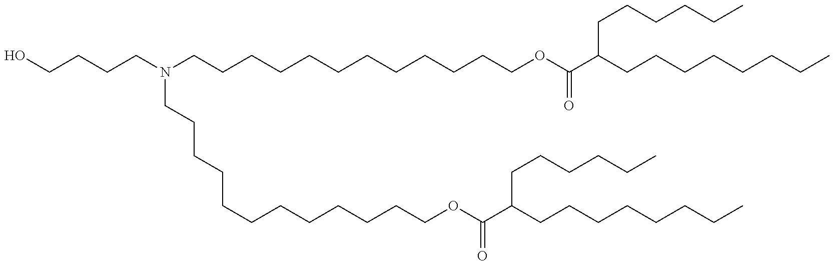 [SMILES: CCCCCCCCC(CCCCCC)C(=O)OCCCCCCCCCCCCN(CCCCO)CCCCCCCCCCCCOC(=O)C(CCCCCC)CCCCCCCC] |
| 24 | 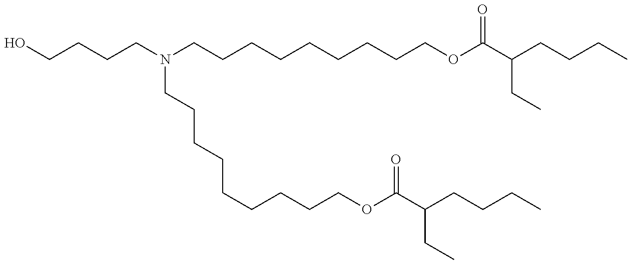 [SMILES: CCCCC(CC)C(=O)OCCCCCCCCCN(CCCCO)CCCCCCCCCOC(=O)C(CC)CCCC] |
| 25 | 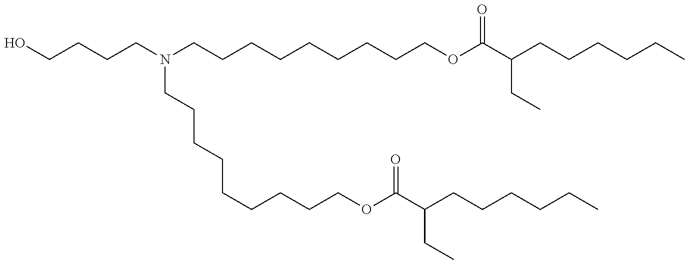 [SMILES: CCCCCCC(CC)C(=O)OCCCCCCCCCN(CCCCO)CCCCCCCCCOC(=O)C(CC)CCCCCC] |
| 26 | 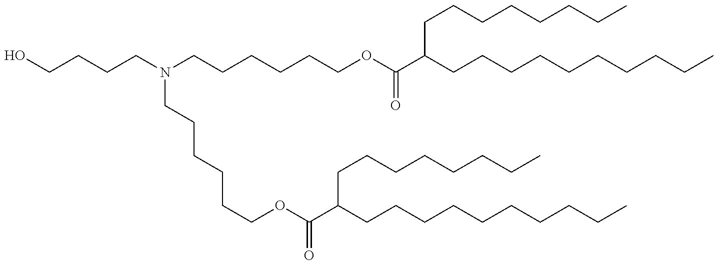 [SMILES: CCCCCCCCCCC(CCCCCCCC)C(=O)OCCCCCCN(CCCCO)CCCCCCOC(=O)C(CCCCCCCC)CCCCCCCCCC] |
| 27 | 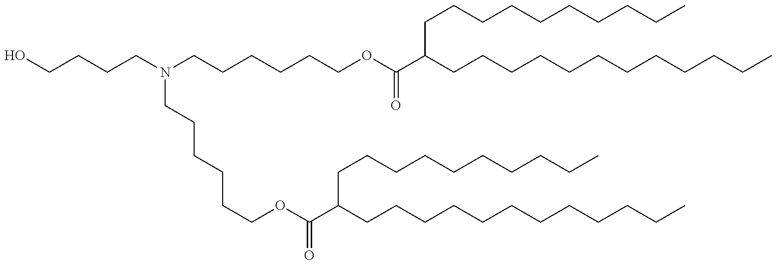 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| 28 | 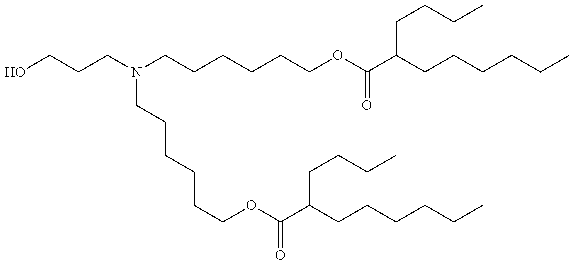 [SMILES: CCCCCCC(CCCC)C(=O)OCCCCCCN(CCCO)CCCCCCOC(=O)C(CCCC)CCCCCC] |
| 29 | 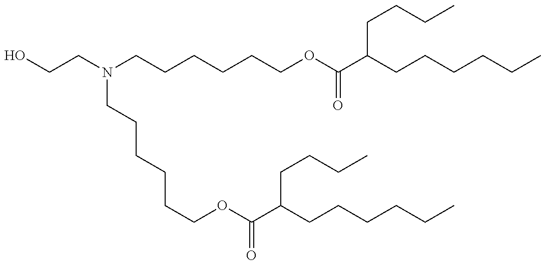 [SMILES: CCCCCCC(CCCC)C(=O)OCCCCCCN(CCO)CCCCCCOC(=O)C(CCCC)CCCCCC] |
| 30 | 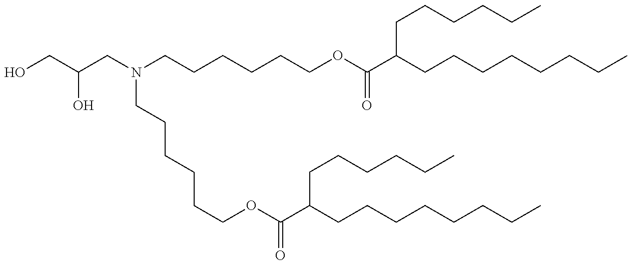 [SMILES: CCCCCCCCC(CCCCCC)C(=O)OCCCCCCN(CCCCCCOC(=O)C(CCCCCC)CCCCCCCC)CC(O)CO] |
| 31 | 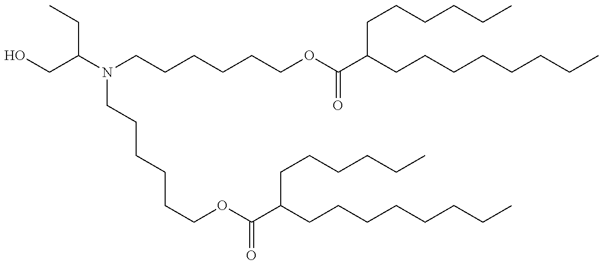 [SMILES: CCCCCCCCC(CCCCCC)C(=O)OCCCCCCN(CCCCCCOC(=O)C(CCCCCC)CCCCCCCC)C(CC)CO] |
| 32 | 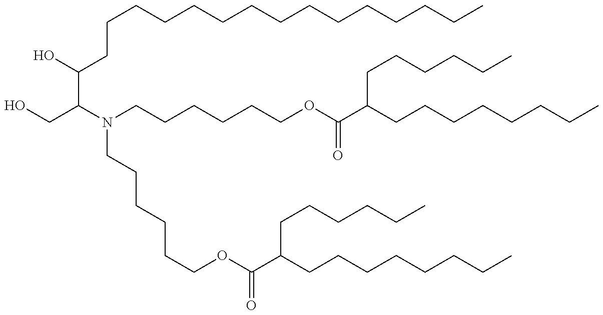 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| 33 | 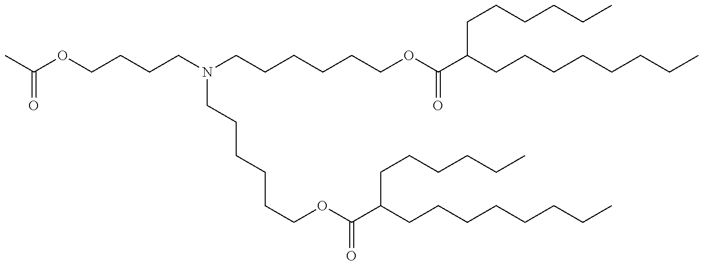 [SMILES: CCCCCCCCC(CCCCCC)C(=O)OCCCCCCN(CCCCCCOC(=O)C(CCCCCC)CCCCCCCC)CCCCOC(C)=O] |
| 34 | 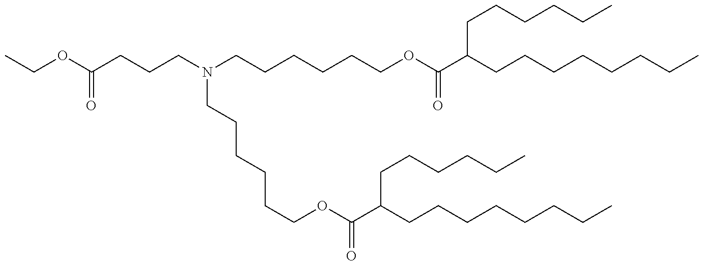 [SMILES: CCCCCCCCC(CCCCCC)C(=O)OCCCCCCN(CCCCCCOC(=O)C(CCCCCC)CCCCCCCC)CCCC(=O)OCC] |
| 35 | 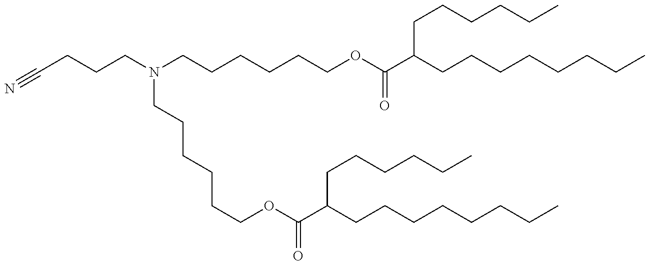 [SMILES: CCCCCCCCC(CCCCCC)C(=O)OCCCCCCN(CCCC#N)CCCCCCOC(=O)C(CCCCCC)CCCCCCCC] |
| 36 | 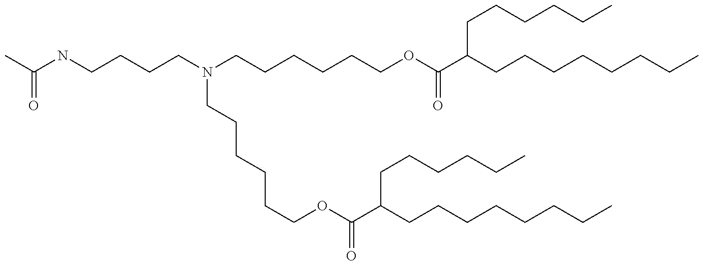 [SMILES: CCCCCCCCC(CCCCCC)C(=O)OCCCCCCN(CCCCCCOC(=O)C(CCCCCC)CCCCCCCC)CCCCNC(C)=O] |
| 37 | 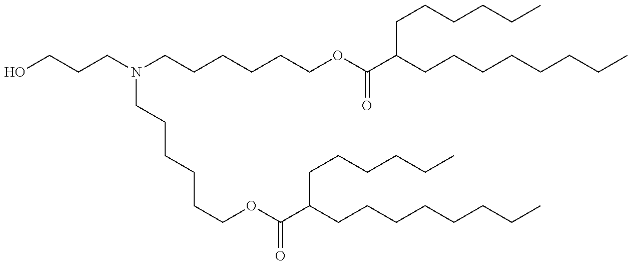 [SMILES: CCCCCCCCC(CCCCCC)C(=O)OCCCCCCN(CCCO)CCCCCCOC(=O)C(CCCCCC)CCCCCCCC] |
| 38 | 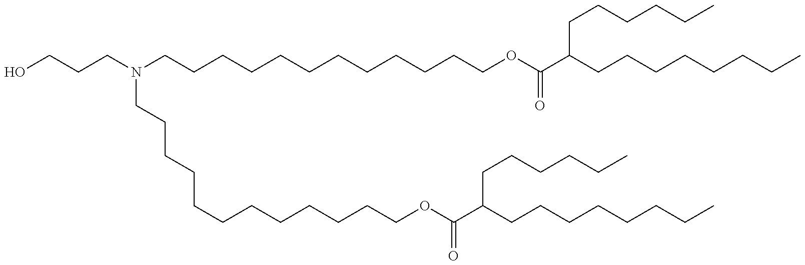 [SMILES: CCCCCCCCC(CCCCCC)C(=O)OCCCCCCCCCCCCN(CCCO)CCCCCCCCCCCCOC(=O)C(CCCCCC)CCCCCCCC] |
| 39 | 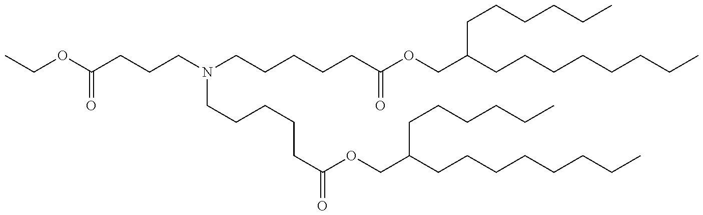 [SMILES: CCCCCCCCC(CCCCCC)COC(=O)CCCCCN(CCCCCC(=O)OCC(CCCCCC)CCCCCCCC)CCCC(=O)OCC] |
| 40 | 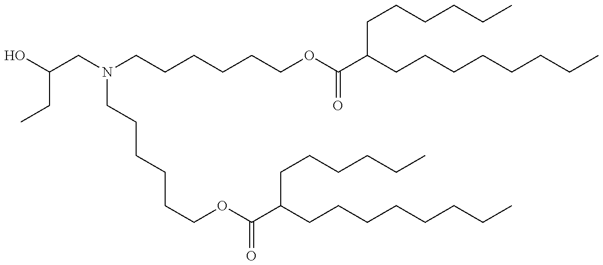 [SMILES: CCCCCCCCC(CCCCCC)C(=O)OCCCCCCN(CCCCCCOC(=O)C(CCCCCC)CCCCCCCC)CC(O)CC] |
| 41 | 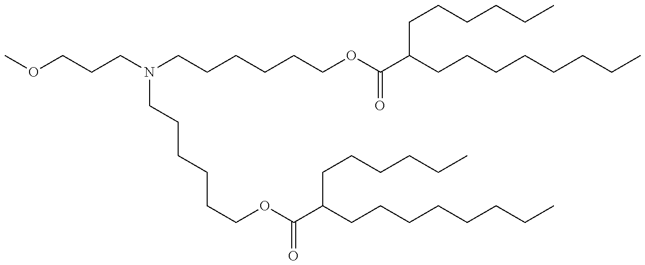 [SMILES: CCCCCCCCC(CCCCCC)C(=O)OCCCCCCN(CCCCCCOC(=O)C(CCCCCC)CCCCCCCC)CCCOC] |
| 42 | 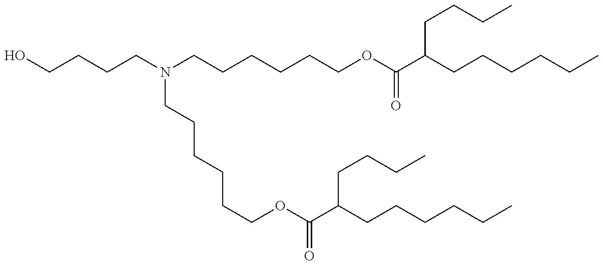 [SMILES: CCCCCCC(CCCC)C(=O)OCCCCCCN(CCCCO)CCCCCCOC(=O)C(CCCC)CCCCCC] |
| 43 | 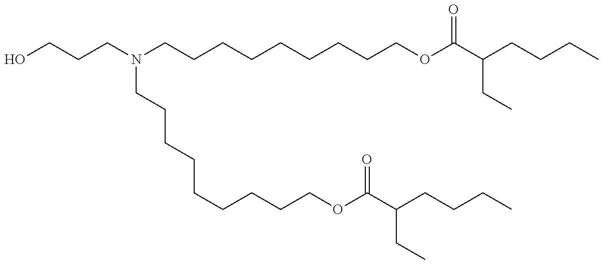 [SMILES: CCCCC(CC)C(=O)OCCCCCCCCCN(CCCO)CCCCCCCCCOC(=O)C(CC)CCCC] |
| 44 | 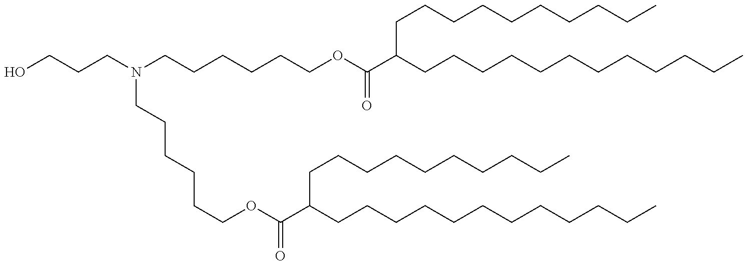 [SMILES: CCCCCCCCCCCCC(CCCCCCCCCC)C(=O)OCCCCCCN(CCCO)CCCCCCOC(=O)C(CCCCCCCCCC)CCCCCCCCCCCC] |
| 45 | 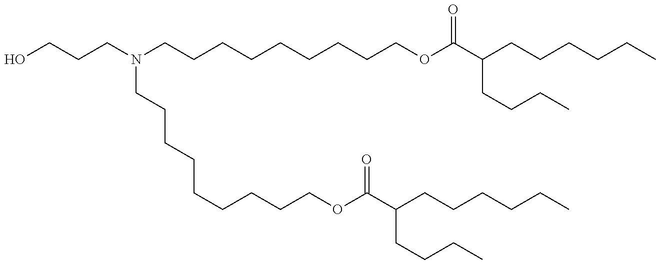 [SMILES: CCCCCCC(CCCC)C(=O)OCCCCCCCCCN(CCCO)CCCCCCCCCOC(=O)C(CCCC)CCCCCC] |
| 46 | 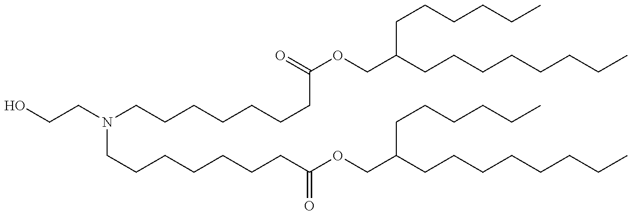 [SMILES: CCCCCCCCC(CCCCCC)COC(=O)CCCCCCCN(CCO)CCCCCCCC(=O)OCC(CCCCCC)CCCCCCCC] |
| 47 | 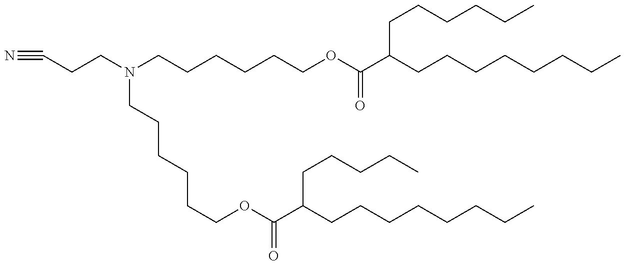 [SMILES: CCCCCCCCC(CCCCC)C(=O)OCCCCCCN(CCC#N)CCCCCCOC(=O)C(CCCCCC)CCCCCCCC] |
| 48 | 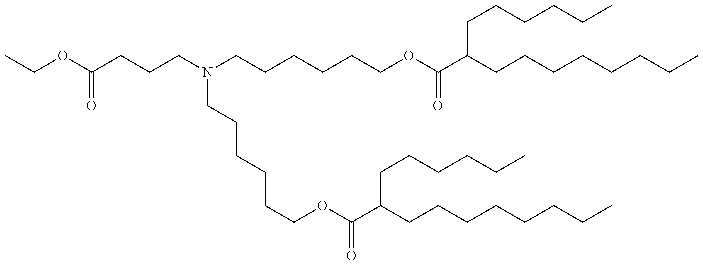 [SMILES: CCCCCCCCC(CCCCCC)C(=O)OCCCCCCN(CCCCCCOC(=O)C(CCCCCC)CCCCCCCC)CCCC(=O)OCC] |
| 49 | 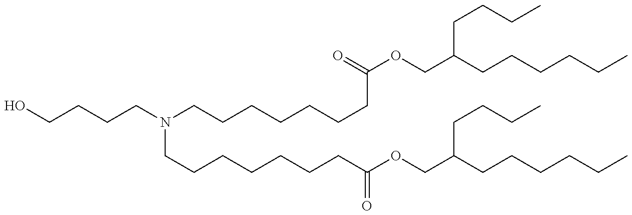 [SMILES: CCCCCCC(CCCC)COC(=O)CCCCCCCN(CCCCO)CCCCCCCC(=O)OCC(CCCC)CCCCCC] |

In some embodiments, the ionizable lipid has one of the structures set forth in Table 11 below. In some embodiments, the ionizable lipid as set forth in Table 11 is as described in international patent application PCT/US2010/061058.

TABLE 11

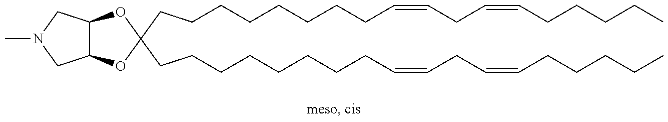 [SMILES: CCCCC/C=C\C/C=C\CCCCCCCCC1(CCCCCCCC/C=C\C/C=C\CCCCC)O[C@H]2CN(C)C[C@H]2O1]

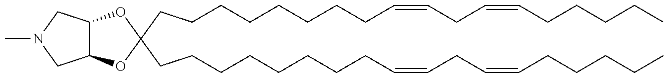 [SMILES: CCCCC/C=C\C/C=C\CCCCCCCCC1(CCCCCCCC/C=C\C/C=C\CCCCC)O[C@H]2CN(C)C[C@@H]2O1]

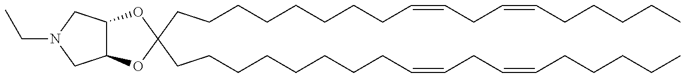 [SMILES: CCCCC/C=C\C/C=C\CCCCCCCCC1(CCCCCCCC/C=C\C/C=C\CCCCC)O[C@H]2CN(CC)C[C@@H]2O1]

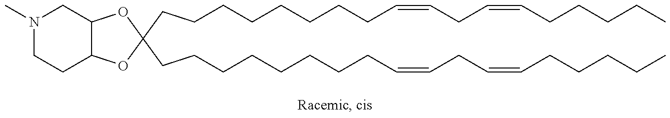 [SMILES: CCCCC/C=C\C/C=C\CCCCCCCCC1(CCCCCCCC/C=C\C/C=C\CCCCC)OC2CCN(C)CC2O1]

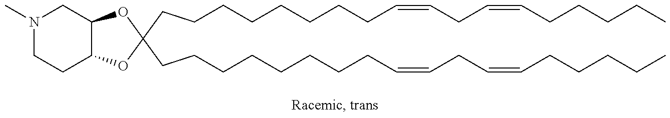 [SMILES: CCCCC/C=C\C/C=C\CCCCCCCCC1(CCCCCCCC/C=C\C/C=C\CCCCC)O[C@@H]2CCN(C)C[C@H]2O1]

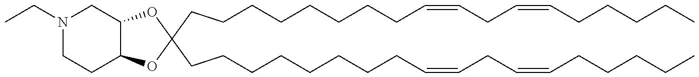 [SMILES: CCCCC/C=C\C/C=C\CCCCCCCCC1(CCCCCCCC/C=C\C/C=C\CCCCC)O[C@H]2CCN(CC)C[C@@H]2O1]

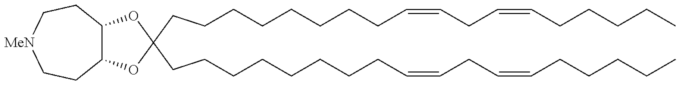 [SMILES: CCCCC/C=C\C/C=C\CCCCCCCCC1(CCCCCCCC/C=C\C/C=C\CCCCC)O[C@H]2CCN(C)CC[C@H]2O1]

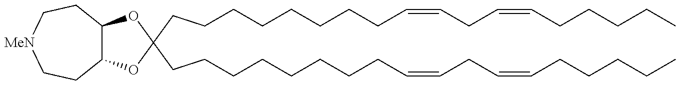 [SMILES: CCCCC/C=C\C/C=C\CCCCCCCCC1(CCCCCCCC/C=C\C/C=C\CCCCC)O[C@@H]2CCN(C)CC[C@H]2O1]

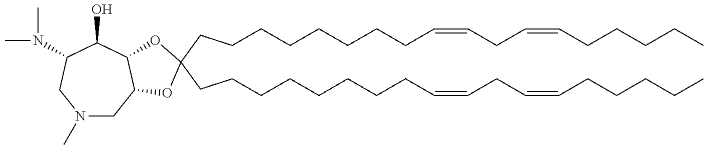 [SMILES: CCCCC/C=C\C/C=C\CCCCCCCCC1(CCCCCCCC/C=C\C/C=C\CCCCC)O[C@H]2[C@H](O)[C@@H](N(C)C)CN(C)C[C@H]2O1]

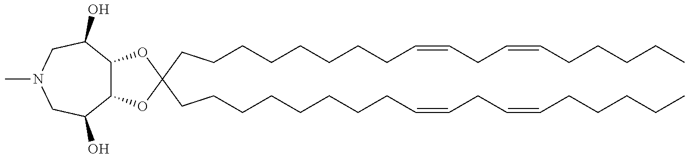 [SMILES: CCCCC/C=C\C/C=C\CCCCCCCCC1(CCCCCCCC/C=C\C/C=C\CCCCC)O[C@@H]2[C@H](O1)[C@@H](O)CN(C)C[C@H]2O]

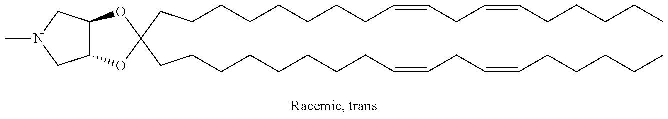 [SMILES: CCCCC/C=C\C/C=C\CCCCCCCCC1(CCCCCCCC/C=C\C/C=C\CCCCC)O[C@@H]2CN(C)C[C@H]2O1]

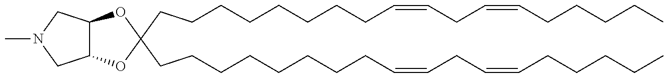 [SMILES: CCCCC/C=C\C/C=C\CCCCCCCCC1(CCCCCCCC/C=C\C/C=C\CCCCC)O[C@@H]2CN(C)C[C@H]2O1]

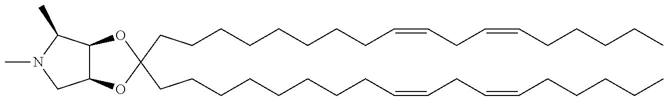 [SMILES: CCCCC/C=C\C/C=C\CCCCCCCCC1(CCCCCCCC/C=C\C/C=C\CCCCC)O[C@H]2[C@H](CN(C)[C@H]2C)O1]

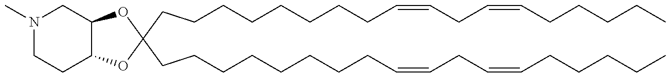 [SMILES: CCCCC/C=C\C/C=C\CCCCCCCCC1(CCCCCCCC/C=C\C/C=C\CCCCC)O[C@@H]2CCN(C)C[C@H]2O1]

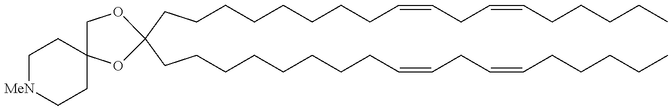 [SMILES: CCCCC/C=C\C/C=C\CCCCCCCCC1(CCCCCCCC/C=C\C/C=C\CCCCC)OCC2(CCN(C)CC2)O1]

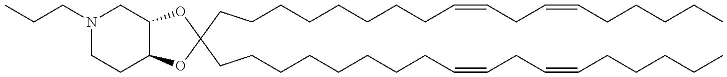 [SMILES: CCCCC/C=C\C/C=C\CCCCCCCCC1(CCCCCCCC/C=C\C/C=C\CCCCC)O[C@H]2CCN(CCC)C[C@@H]2O1]

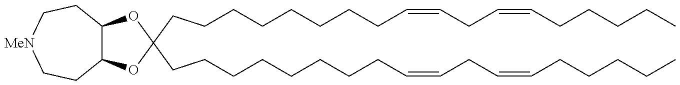 [SMILES: CCCCC/C=C\C/C=C\CCCCCCCCC1(CCCCCCCC/C=C\C/C=C\CCCCC)O[C@H]2CCN(C)CC[C@H]2O1]

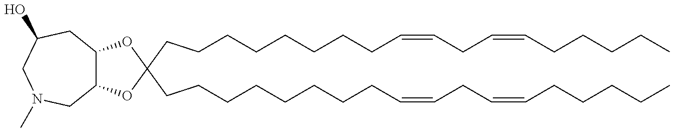 [SMILES: CCCCC/C=C\C/C=C\CCCCCCCCC1(CCCCCCCC/C=C\C/C=C\CCCCC)O[C@H]2C[C@H](O)CN(C)C[C@H]2O1]

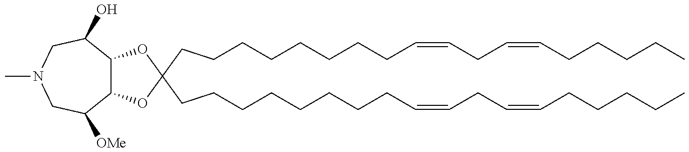 [SMILES: CCCCC/C=C\C/C=C\CCCCCCCCC1(CCCCCCCC/C=C\C/C=C\CCCCC)O[C@@H]2[C@H](O1)[C@@H](OC)CN(C)C[C@H]2O]

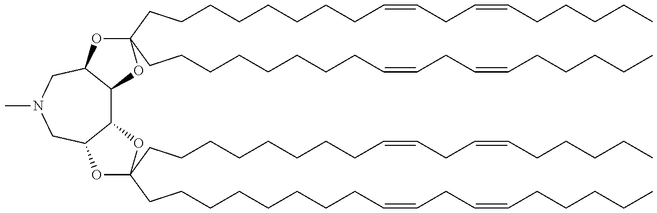 [SMILES: CCCCC/C=C\C/C=C\CCCCCCCCC1(CCCCCCCC/C=C\C/C=C\CCCCC)O[C@H]2[C@@H]3OC(CCCCCCCC/C=C\C/C=C\CCCCC)(CCCCCCCC/C=C\C/C=C\CCCCC)O[C@@H]3CN(C)C[C@H]2O1]

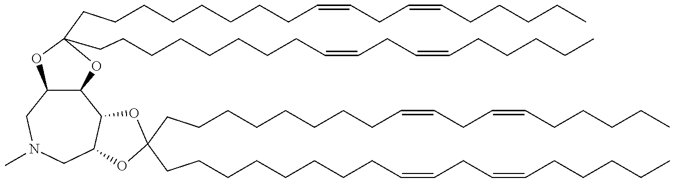 [SMILES: CCCCC/C=C\C/C=C\CCCCCCCCC1(CCCCCCCC/C=C\C/C=C\CCCCC)O[C@H]2[C@@H]3OC(CCCCCCCC/C=C\C/C=C\CCCCC)(CCCCCCCC/C=C\C/C=C\CCCCC)O[C@@H]3CN(C)C[C@H]2O1]

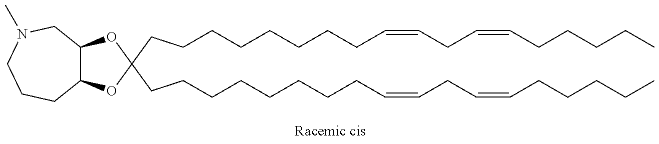 [SMILES: CCCCC/C=C\C/C=C\CCCCCCCCC1(CCCCCCCC/C=C\C/C=C\CCCCC)O[C@H]2CCCN(C)C[C@H]2O1]

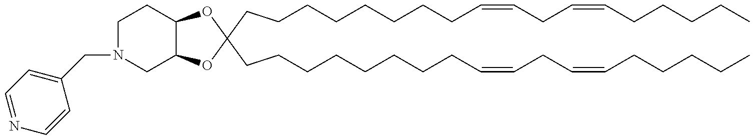 [SMILES: CCCCC/C=C\C/C=C\CCCCCCCCC1(CCCCCCCC/C=C\C/C=C\CCCCC)O[C@H]2CN(Cc3ccncc3)CC[C@H]2O1]

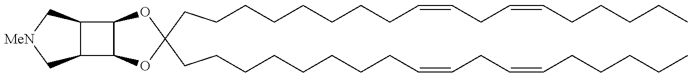 [SMILES: CCCCC/C=C\C/C=C\CCCCCCCCC1(CCCCCCCC/C=C\C/C=C\CCCCC)O[C@@H]2[C@@H]3CN(C)C[C@@H]3[C@@H]2O1]

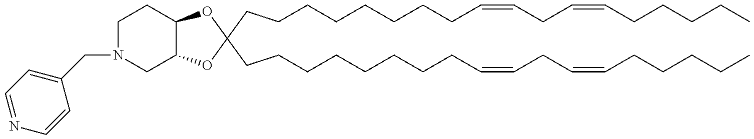 [SMILES: CCCCC/C=C\C/C=C\CCCCCCCCC1(CCCCCCCC/C=C\C/C=C\CCCCC)O[C@@H]2CCN(Cc3ccncc3)C[C@H]2O1]

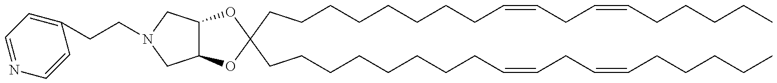 [SMILES: CCCCC/C=C\C/C=C\CCCCCCCCC1(CCCCCCCC/C=C\C/C=C\CCCCC)O[C@H]2CN(CCc3ccncc3)C[C@@H]2O1]

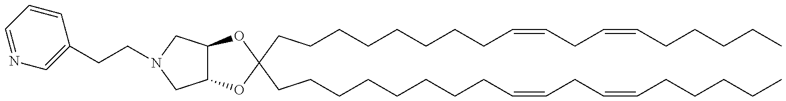 [SMILES: CCCCC/C=C\C/C=C\CCCCCCCCC1(CCCCCCCC/C=C\C/C=C\CCCCC)O[C@@H]2CN(CCc3cccnc3)C[C@H]2O1]

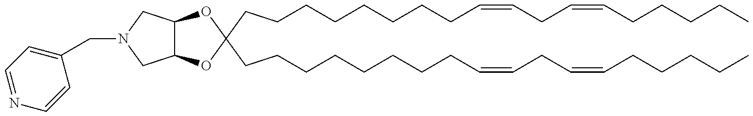 [SMILES: CCCCC/C=C\C/C=C\CCCCCCCCC1(CCCCCCCC/C=C\C/C=C\CCCCC)O[C@H]2CN(Cc3ccncc3)C[C@H]2O1]

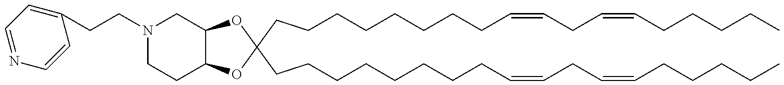 [SMILES: CCCCC/C=C\C/C=C\CCCCCCCCC1(CCCCCCCC/C=C\C/C=C\CCCCC)O[C@H]2CCN(CCc3ccncc3)C[C@H]2O1]

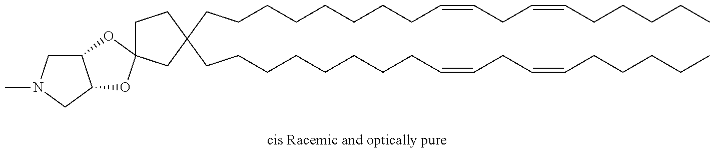

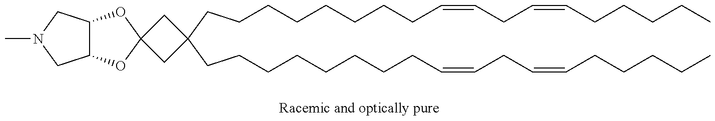 [SMILES: CCCCC/C=C\C/C=C\CCCCCCCCC1(CCCCCCCC/C=C\C/C=C\CCCCC)CC2(C1)O[C@H]1CN(C)C[C@H]1O2]

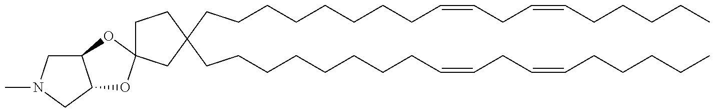 [SMILES: CCCCC/C=C\C/C=C\CCCCCCCCC1(CCCCCCCC/C=C\C/C=C\CCCCC)CCC2(C1)O[C@@H]1CN(C)C[C@H]1O2]

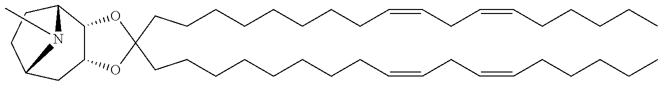 [SMILES: CCCCC/C=C\C/C=C\CCCCCCCCC1(CCCCCCCC/C=C\C/C=C\CCCCC)O[C@H]2[C@H]3CC[C@@H](C[C@H]2O1)N3C]

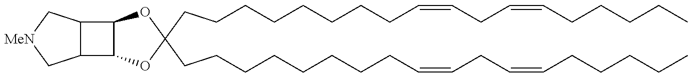 [SMILES: CCCCC/C=C\C/C=C\CCCCCCCCC1(CCCCCCCC/C=C\C/C=C\CCCCC)O[C@@H]2C3CN(C)CC3[C@H]2O1]

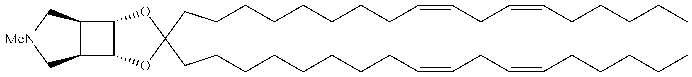 [SMILES: CCCCC/C=C\C/C=C\CCCCCCCCC1(CCCCCCCC/C=C\C/C=C\CCCCC)O[C@@H]2[C@H]3CN(C)C[C@H]3[C@@H]2O1]

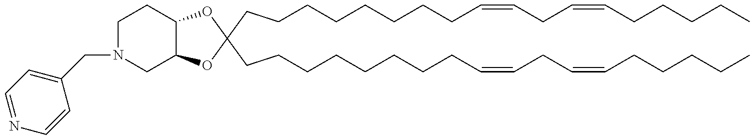 [SMILES: CCCCC/C=C\C/C=C\CCCCCCCCC1(CCCCCCCC/C=C\C/C=C\CCCCC)O[C@H]2CCN(Cc3ccncc3)C[C@@H]2O1]

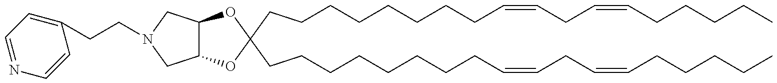 [SMILES: CCCCC/C=C\C/C=C\CCCCCCCCC1(CCCCCCCC/C=C\C/C=C\CCCCC)O[C@@H]2CN(CCc3ccncc3)C[C@H]2O1]

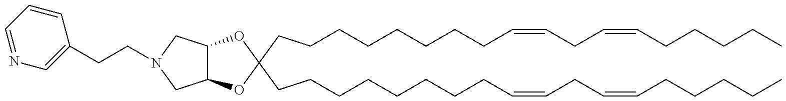 [SMILES: CCCCC/C=C\C/C=C\CCCCCCCCC1(CCCCCCCC/C=C\C/C=C\CCCCC)O[C@H]2CN(CCc3cccnc3)C[C@@H]2O1]

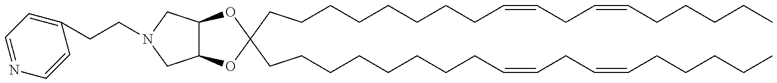 [SMILES: CCCCC/C=C\C/C=C\CCCCCCCCC1(CCCCCCCC/C=C\C/C=C\CCCCC)O[C@H]2CN(CCc3ccncc3)C[C@H]2O1]

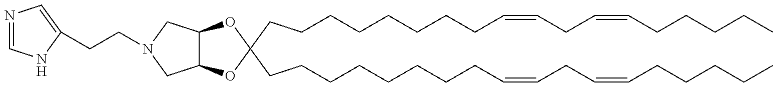 [SMILES: CCCCC/C=C\C/C=C\CCCCCCCCC1(CCCCCCCC/C=C\C/C=C\CCCCC)O[C@H]2CN(CCc3cnc[nH]3)C[C@H]2O1]

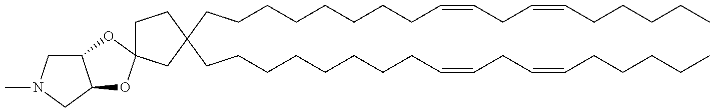 [SMILES: CCCCC/C=C\C/C=C\CCCCCCCCC1(CCCCCCCC/C=C\C/C=C\CCCCC)CCC2(C1)O[C@H]1CN(C)C[C@@H]1O2]

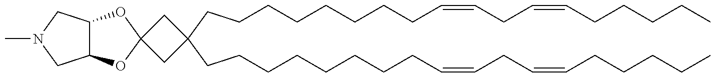 [SMILES: CCCCC/C=C\C/C=C\CCCCCCCCC1(CCCCCCCC/C=C\C/C=C\CCCCC)CC2(C1)O[C@H]1CN(C)C[C@@H]1O2]

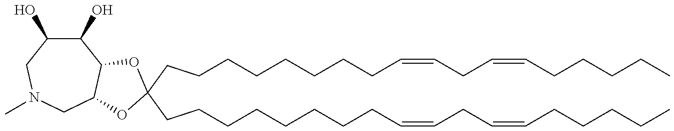 [SMILES: CCCCC/C=C\C/C=C\CCCCCCCCC1(CCCCCCCC/C=C\C/C=C\CCCCC)O[C@H]2[C@H](O)[C@H](O)CN(C)C[C@H]2O1]

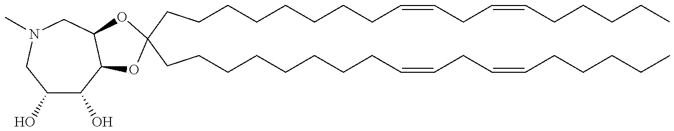 [SMILES: CCCCC/C=C\C/C=C\CCCCCCCCC1(CCCCCCCC/C=C\C/C=C\CCCCC)O[C@H]2[C@H](O)[C@H](O)CN(C)C[C@H]2O1]

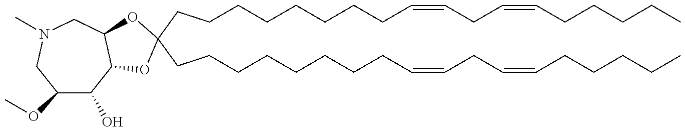 [SMILES: CCCCC/C=C\C/C=C\CCCCCCCCC1(CCCCCCCC/C=C\C/C=C\CCCCC)O[C@@H]2[C@H](O)[C@@H](OC)CN(C)C[C@H]2O1]

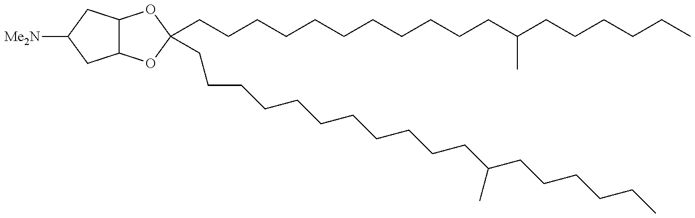 [SMILES: CCCCCCC(C)CCCCCCCCCCCC1(CCCCCCCCCCCC(C)CCCCCC)OC2CC(N(C)C)CC2O1]

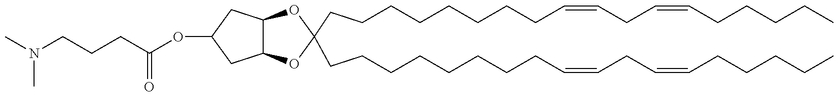 [SMILES: CCCCC/C=C\C/C=C\CCCCCCCCC1(CCCCCCCC/C=C\C/C=C\CCCCC)O[C@H]2CC(OC(=O)CCCN(C)C)C[C@H]2O1]

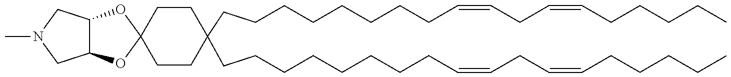 [SMILES: CCCCC/C=C\C/C=C\CCCCCCCCC1(CCCCCCCC/C=C\C/C=C\CCCCC)CCC2(CC1)O[C@H]1CN(C)C[C@@H]1O2]

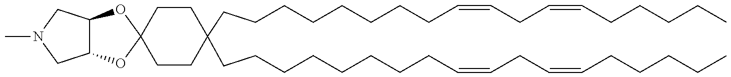 [SMILES: CCCCC/C=C\C/C=C\CCCCCCCCC1(CCCCCCCC/C=C\C/C=C\CCCCC)CCC2(CC1)O[C@@H]1CN(C)C[C@H]1O2]

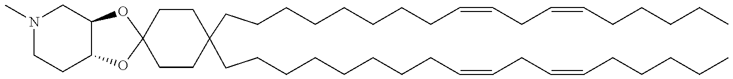 [SMILES: CCCCC/C=C\C/C=C\CCCCCCCCC1(CCCCCCCC/C=C\C/C=C\CCCCC)CCC2(CC1)O[C@@H]1CCN(C)C[C@H]1O2]

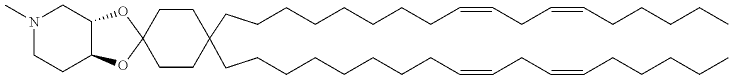 [SMILES: CCCCC/C=C\C/C=C\CCCCCCCCC1(CCCCCCCC/C=C\C/C=C\CCCCC)CCC2(CC1)O[C@H]1CCN(C)C[C@@H]1O2]

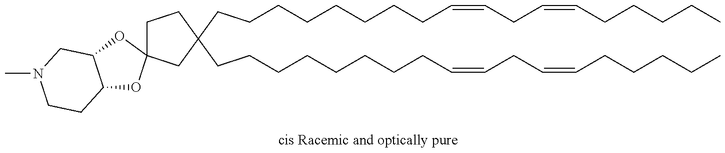

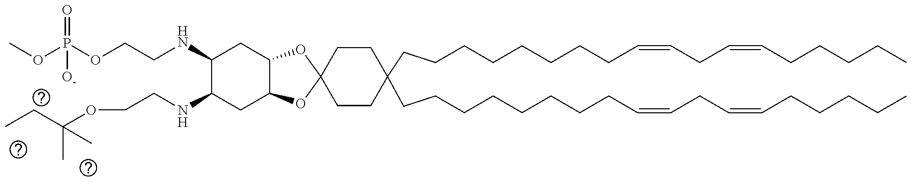

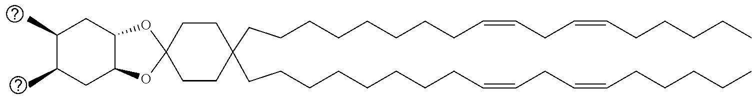 [SMILES: CCCCC/C=C\C/C=C\CCCCCCCCC1(CCCCCCCC/C=C\C/C=C\CCCCC)CCC2(CC1)O[C@H]1C[C@@H](C)[C@@H](C)C[C@@H]1O2]

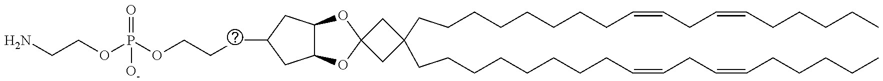

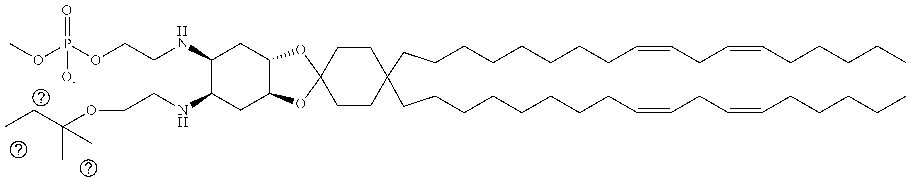

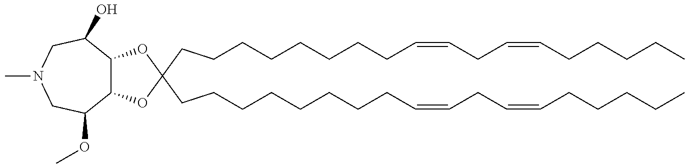 [SMILES: CCCCC/C=C\C/C=C\CCCCCCCCC1(CCCCCCCC/C=C\C/C=C\CCCCC)O[C@@H]2[C@H](O1)[C@@H](OC)CN(C)C[C@H]2O]

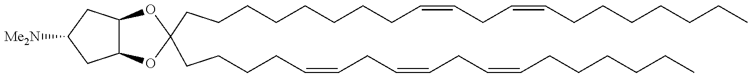

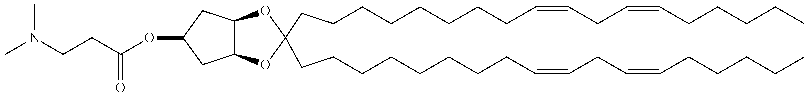 [SMILES: CCCCC/C=C\C/C=C\CCCCCCCCC1(CCCCCCCC/C=C\C/C=C\CCCCC)O[C@H]2C[C@@H](OC(=O)CCN(C)C)C[C@H]2O1]

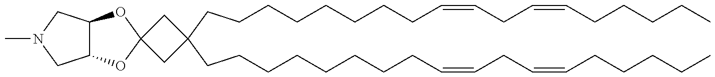 [SMILES: CCCCC/C=C\C/C=C\CCCCCCCCC1(CCCCCCCC/C=C\C/C=C\CCCCC)CC2(C1)O[C@@H]1CN(C)C[C@H]1O2]

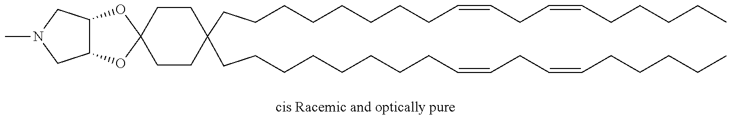 [SMILES: CCCCC/C=C\C/C=C\CCCCCCCCC1(CCCCCCCC/C=C\C/C=C\CCCCC)CCC2(CC1)O[C@H]1CN(C)C[C@H]1O2]

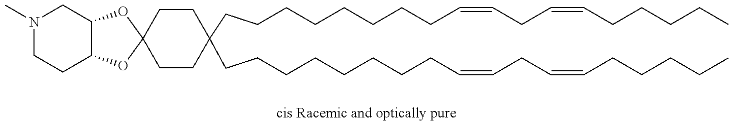 [SMILES: CCCCC/C=C\C/C=C\CCCCCCCCC1(CCCCCCCC/C=C\C/C=C\CCCCC)CCC2(CC1)O[C@H]1CN(C)CC[C@H]1O2]

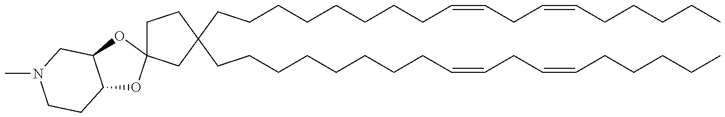

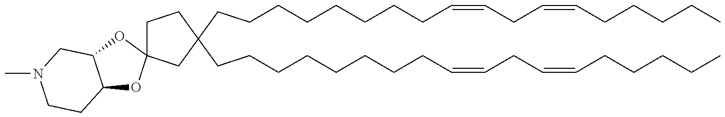

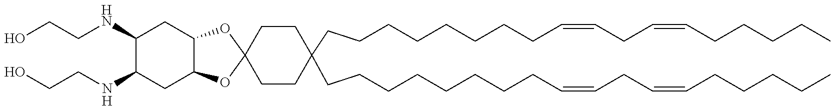 [SMILES: CCCCC/C=C\C/C=C\CCCCCCCCC1(CCCCCCCC/C=C\C/C=C\CCCCC)CCC2(CC1)O[C@H]1C[C@H](NCCO)[C@H](NCCO)C[C@@H]1O2]

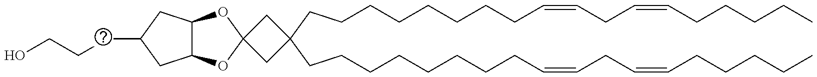 [SMILES: CCCCC/C=C\C/C=C\CCCCCCCCC1(CCCCCCCC/C=C\C/C=C\CCCCC)CC2(C1)O[C@H]1CC(CCCO)C[C@H]1O2]

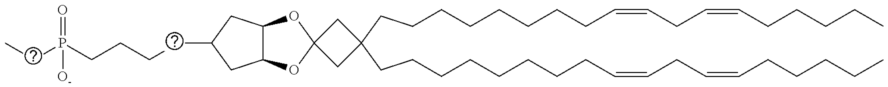

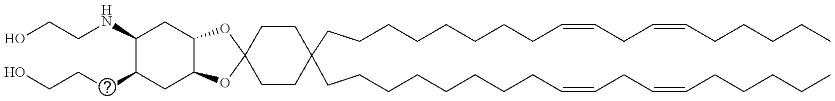 [SMILES: CCCCC/C=C\C/C=C\CCCCCCCCC1(CCCCCCCC/C=C\C/C=C\CCCCC)CCC2(CC1)O[C@H]1C[C@@H](CCCO)[C@@H](NCCO)C[C@@H]1O2]

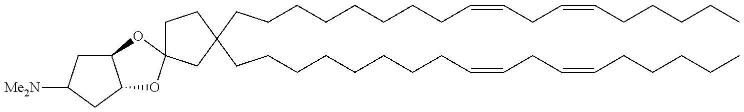 [SMILES: CCCCC/C=C\C/C=C\CCCCCCCCC1(CCCCCCCC/C=C\C/C=C\CCCCC)CCC2(C1)O[C@@H]1CC(N(C)C)C[C@H]1O2]

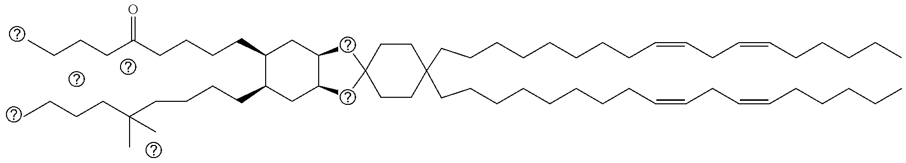 [SMILES: CCCCC/C=C\C/C=C\CCCCCCCCC1(CCCCCCCC/C=C\C/C=C\CCCCC)CCC2(CC1)C[C@H]1C[C@@H](CCCCC(C)(C)CCCC)[C@@H](CCCCC(=O)CCCC)C[C@H]1C2]

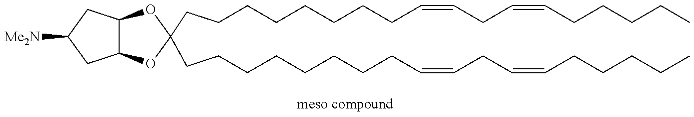 [SMILES: CCCCC/C=C\C/C=C\CCCCCCCCC1(CCCCCCCC/C=C\C/C=C\CCCCC)O[C@H]2C[C@@H](N(C)C)C[C@H]2O1]

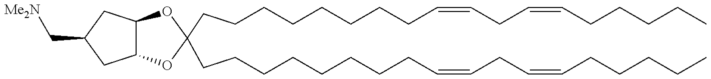

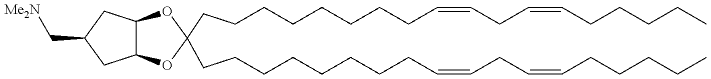 [SMILES: CCCCC/C=C\C/C=C\CCCCCCCCC1(CCCCCCCC/C=C\C/C=C\CCCCC)O[C@H]2C[C@@H](CN(C)C)C[C@H]2O1]

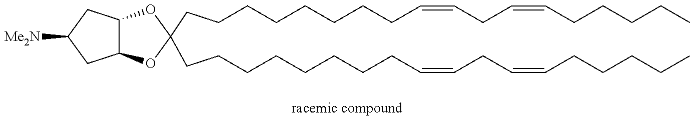

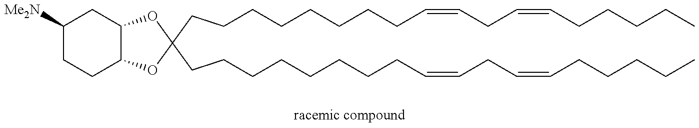 [SMILES: CCCCC/C=C\C/C=C\CCCCCCCCC1(CCCCCCCC/C=C\C/C=C\CCCCC)O[C@H]2C[C@H](N(C)C)CC[C@H]2O1]

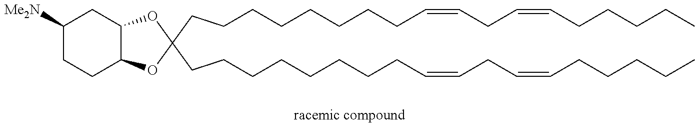 [SMILES: CCCCC/C=C\C/C=C\CCCCCCCCC1(CCCCCCCC/C=C\C/C=C\CCCCC)O[C@H]2CC[C@@H](N(C)C)C[C@@H]2O1]

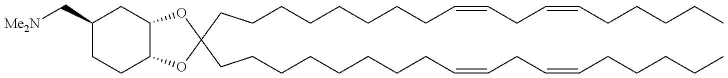 [SMILES: CCCCC/C=C\C/C=C\CCCCCCCCC1(CCCCCCCC/C=C\C/C=C\CCCCC)O[C@H]2C[C@H](CN(C)C)CC[C@H]2O1]

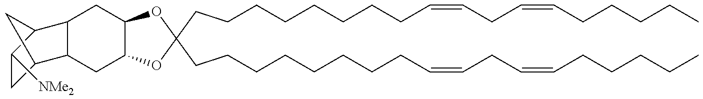 [SMILES: CCCCC/C=C\C/C=C\CCCCCCCCC1(CCCCCCCC/C=C\C/C=C\CCCCC)O[C@@H]2CC3C4CC(C3C[C@H]2O1)C(N(C)C)C4]

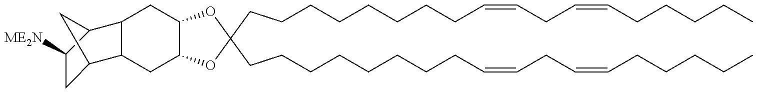

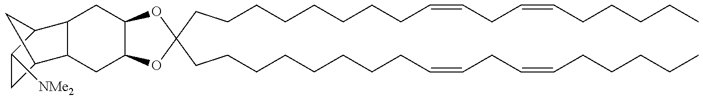 [SMILES: CCCCC/C=C\C/C=C\CCCCCCCCC1(CCCCCCCC/C=C\C/C=C\CCCCC)O[C@H]2CC3C4CC(C3C[C@H]2O1)C(N(C)C)C4]

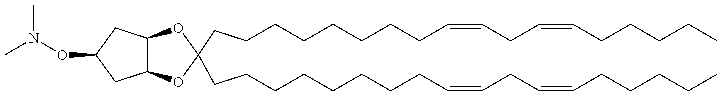 [SMILES: CCCCC/C=C\C/C=C\CCCCCCCCC1(CCCCCCCC/C=C\C/C=C\CCCCC)O[C@H]2C[C@@H](ON(C)C)C[C@H]2O1]

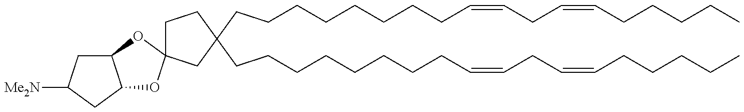 [SMILES: CCCCC/C=C\C/C=C\CCCCCCCCC1(CCCCCCCC/C=C\C/C=C\CCCCC)CCC2(C1)O[C@@H]1CC(N(C)C)C[C@H]1O2]

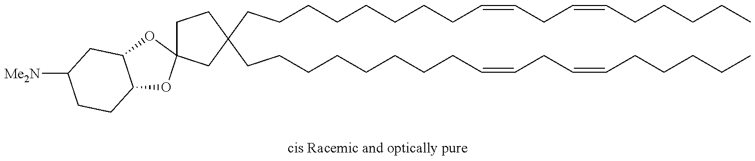

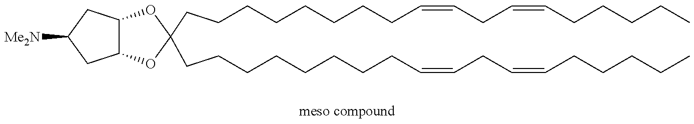 [SMILES: CCCCC/C=C\C/C=C\CCCCCCCCC1(CCCCCCCC/C=C\C/C=C\CCCCC)O[C@H]2C[C@H](N(C)C)C[C@H]2O1]

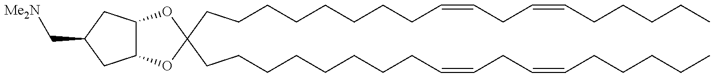 [SMILES: CCCCC/C=C\C/C=C\CCCCCCCCC1(CCCCCCCC/C=C\C/C=C\CCCCC)O[C@H]2C[C@H](CN(C)C)C[C@H]2O1]

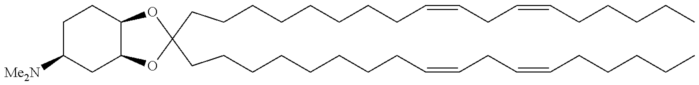 [SMILES: CCCCC/C=C\C/C=C\CCCCCCCCC1(CCCCCCCC/C=C\C/C=C\CCCCC)O[C@H]2C[C@@H](N(C)C)CC[C@H]2O1]

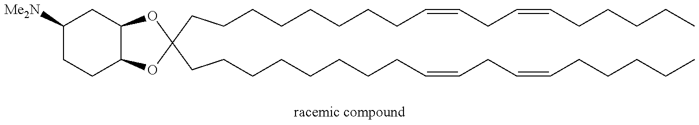 [SMILES: CCCCC/C=C\C/C=C\CCCCCCCCC1(CCCCCCCC/C=C\C/C=C\CCCCC)O[C@H]2CC[C@@H](N(C)C)C[C@H]2O1]

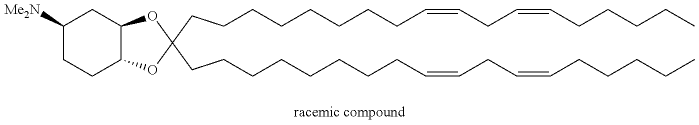 [SMILES: CCCCC/C=C\C/C=C\CCCCCCCCC1(CCCCCCCC/C=C\C/C=C\CCCCC)O[C@@H]2CC[C@@H](N(C)C)C[C@H]2O1]

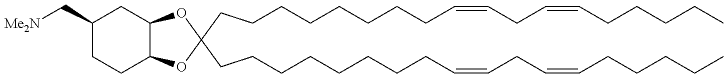 [SMILES: CCCCC/C=C\C/C=C\CCCCCCCCC1(CCCCCCCC/C=C\C/C=C\CCCCC)O[C@H]2CC[C@@H](CN(C)C)C[C@H]2O1]

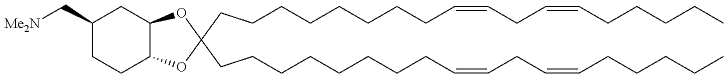 [SMILES: CCCCC/C=C\C/C=C\CCCCCCCCC1(CCCCCCCC/C=C\C/C=C\CCCCC)O[C@@H]2CC[C@@H](CN(C)C)C[C@H]2O1]

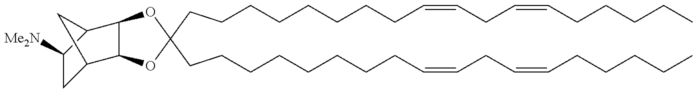 [SMILES: CCCCC/C=C\C/C=C\CCCCCCCCC1(CCCCCCCC/C=C\C/C=C\CCCCC)O[C@@H]2C3CC(C[C@H]3N(C)C)[C@@H]2O1]

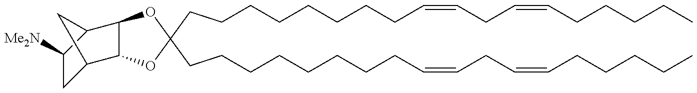 [SMILES: CCCCC/C=C\C/C=C\CCCCCCCCC1(CCCCCCCC/C=C\C/C=C\CCCCC)O[C@@H]2C3CC([C@H]2O1)[C@H](N(C)C)C3]

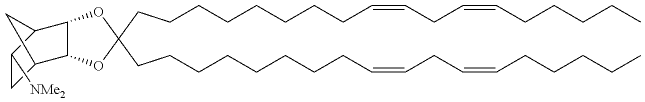 [SMILES: CCCCC/C=C\C/C=C\CCCCCCCCC1(CCCCCCCC/C=C\C/C=C\CCCCC)O[C@@H]2C3CC(C(N(C)C)C3)[C@@H]2O1]

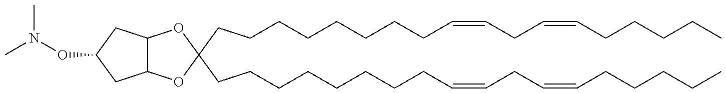

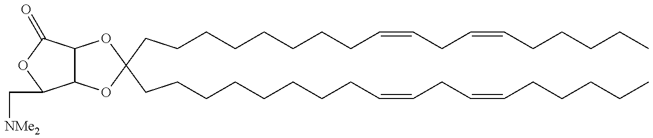 [SMILES: CCCCC/C=C\C/C=C\CCCCCCCCC1(CCCCCCCC/C=C\C/C=C\CCCCC)OC2C(=O)OC(CN(C)C)C2O1]

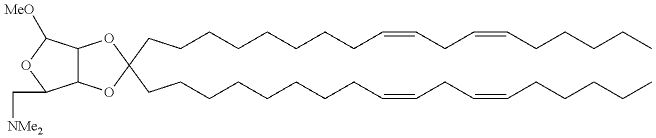 [SMILES: CCCCC/C=C\C/C=C\CCCCCCCCC1(CCCCCCCC/C=C\C/C=C\CCCCC)OC2C(CN(C)C)OC(OC)C2O1]

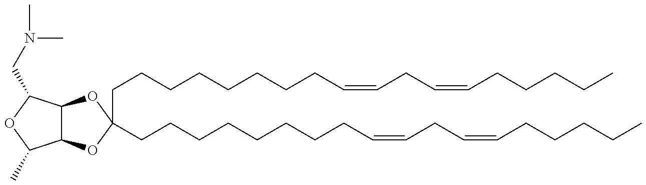 [SMILES: CCCCC/C=C\C/C=C\CCCCCCCCC1(CCCCCCCC/C=C\C/C=C\CCCCC)O[C@@H]2[C@H](O1)[C@@H](CN(C)C)O[C@H]2C]

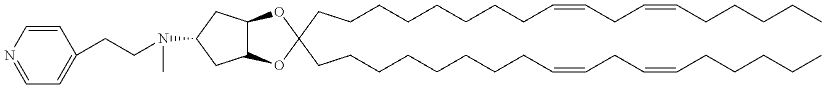

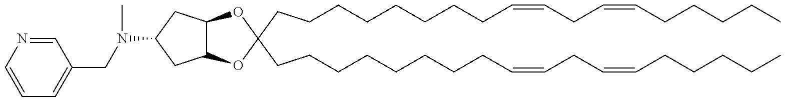

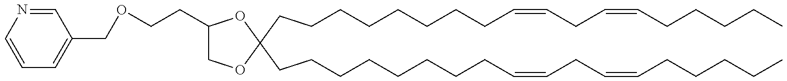 [SMILES: CCCCC/C=C\C/C=C\CCCCCCCCC1(CCCCCCCC/C=C\C/C=C\CCCCC)OCC(CCOCc2cccnc2)O1]

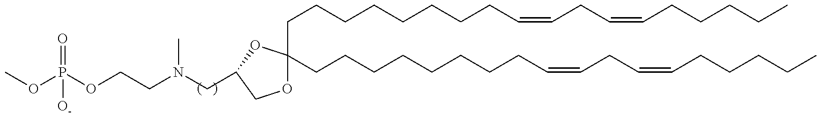

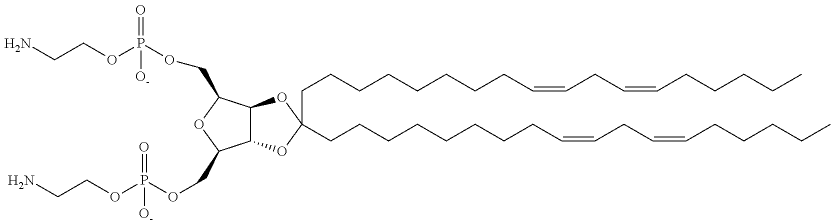

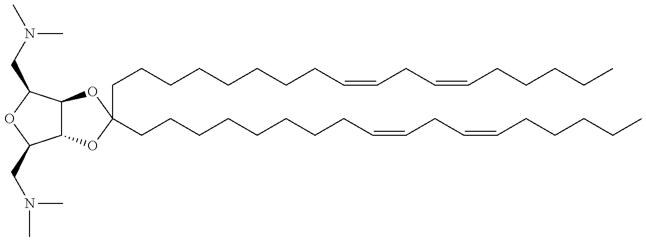 [SMILES: CCCCC/C=C\C/C=C\CCCCCCCCC1(CCCCCCCC/C=C\C/C=C\CCCCC)O[C@H]2[C@H](O1)[C@@H](CN(C)C)O[C@H]2CN(C)C]

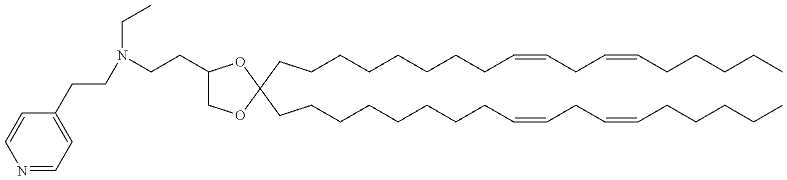 [SMILES: CCCCC/C=C\C/C=C\CCCCCCCCC1(CCCCCCCC/C=C\C/C=C\CCCCC)OCC(CCN(CC)CCc2ccncc2)O1]

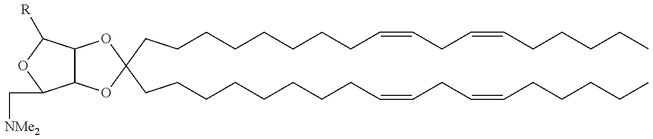

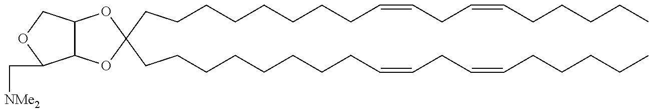 [SMILES: CCCCC/C=C\C/C=C\CCCCCCCCC1(CCCCCCCC/C=C\C/C=C\CCCCC)OC2COC(CN(C)C)C2O1]

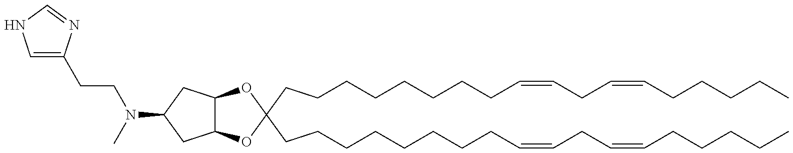 [SMILES: CCCCC/C=C\C/C=C\CCCCCCCCC1(CCCCCCCC/C=C\C/C=C\CCCCC)O[C@H]2C[C@@H](N(C)CCc3c[nH]cn3)C[C@H]2O1]

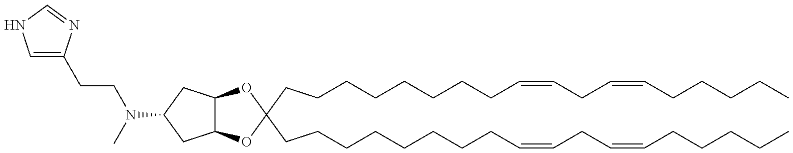 [SMILES: CCCCC/C=C\C/C=C\CCCCCCCCC1(CCCCCCCC/C=C\C/C=C\CCCCC)O[C@H]2C[C@H](N(C)CCc3c[nH]cn3)C[C@H]2O1]

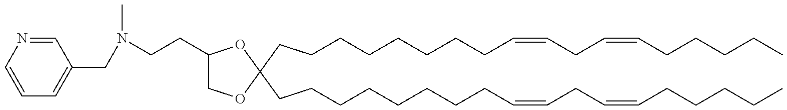 [SMILES: CCCCC/C=C\C/C=C\CCCCCCCCC1(CCCCCCCC/C=C\C/C=C\CCCCC)OCC(CCN(C)Cc2cccnc2)O1]

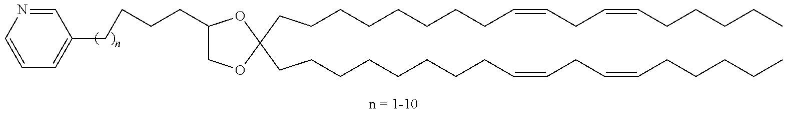

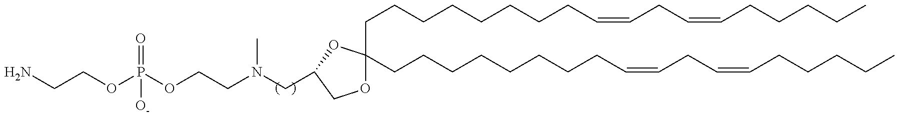

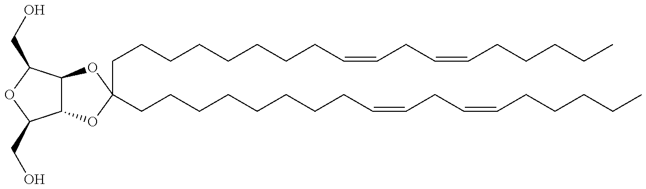 [SMILES: CCCCC/C=C\C/C=C\CCCCCCCCC1(CCCCCCCC/C=C\C/C=C\CCCCC)O[C@H]2[C@H](O1)[C@@H](CO)O[C@H]2CO]

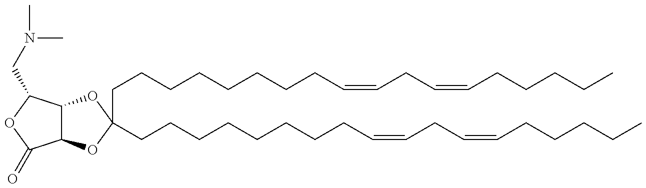 [SMILES: CCCCC/C=C\C/C=C\CCCCCCCCC1(CCCCCCCC/C=C\C/C=C\CCCCC)O[C@H]2[C@@H](CN(C)C)OC(=O)[C@@H]2O1]

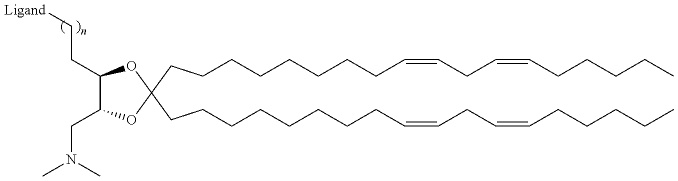

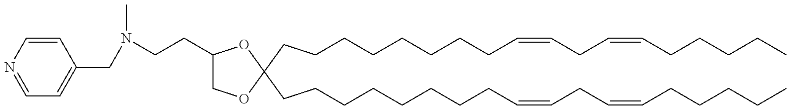 [SMILES: CCCCC/C=C\C/C=C\CCCCCCCCC1(CCCCCCCC/C=C\C/C=C\CCCCC)OCC(CCN(C)Cc2ccncc2)O1]

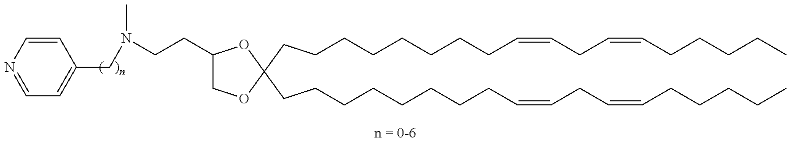

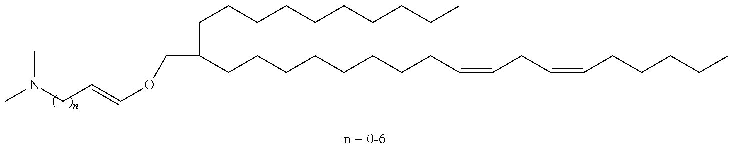

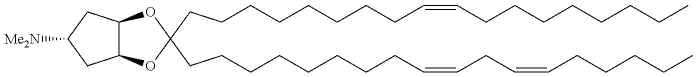

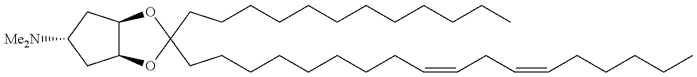

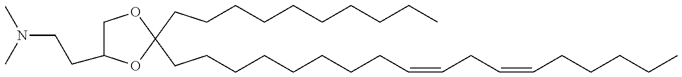 [SMILES: CCCCC/C=C\C/C=C\CCCCCCCCC1(CCCCCCCCCC)OCC(CCN(C)C)O1]

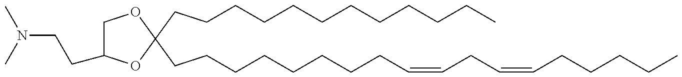 [SMILES: CCCCC/C=C\C/C=C\CCCCCCCCC1(CCCCCCCCCCCC)OCC(CCN(C)C)O1]

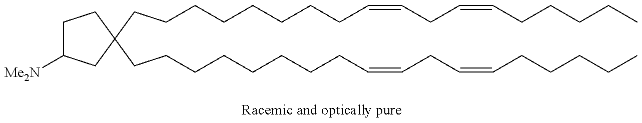 [SMILES: CCCCC/C=C\C/C=C\CCCCCCCCC1(CCCCCCCC/C=C\C/C=C\CCCCC)CCC(N(C)C)C1]

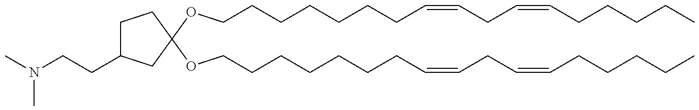 [SMILES: CCCCC/C=C\C/C=C\CCCCCCCOC1(OCCCCCCC/C=C\C/C=C\CCCCC)CCC(CCN(C)C)C1]

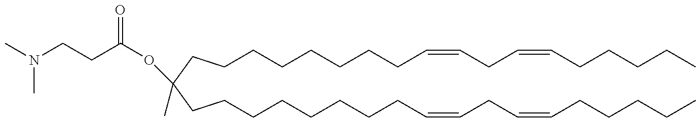 [SMILES: CCCCC/C=C\C/C=C\CCCCCCCCC(C)(CCCCCCCC/C=C\C/C=C\CCCCC)OC(=O)CCN(C)C]

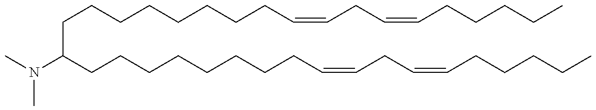 [SMILES: CCCCC/C=C\C/C=C\CCCCCCCCC(CCCCCCCC/C=C\C/C=C\CCCCC)N(C)C]

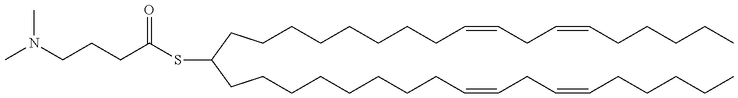 [SMILES: CCCCC/C=C\C/C=C\CCCCCCCCC(CCCCCCCC/C=C\C/C=C\CCCCC)SC(=O)CCCN(C)C]

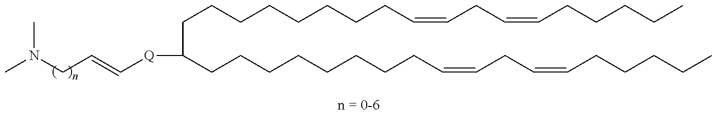

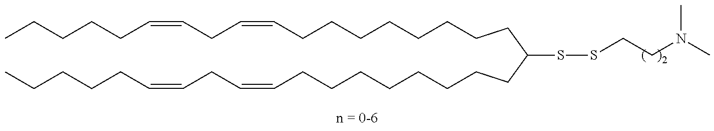

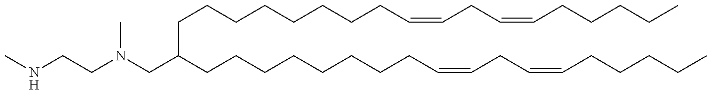 [SMILES: CCCCC/C=C\C/C=C\CCCCCCCCC(CCCCCCCC/C=C\C/C=C\CCCCC)CN(C)CCNC]

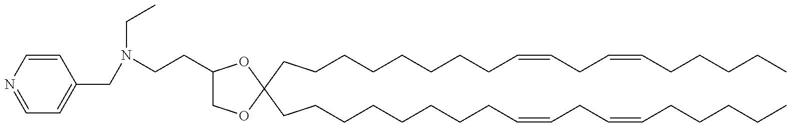 [SMILES: CCCCC/C=C\C/C=C\CCCCCCCCC1(CCCCCCCC/C=C\C/C=C\CCCCC)OCC(CCN(CC)Cc2ccncc2)O1]

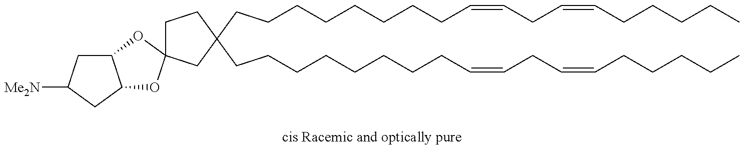

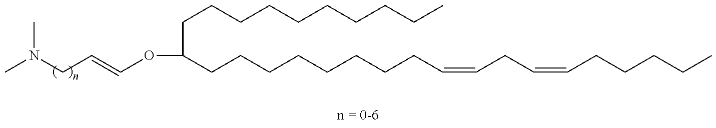

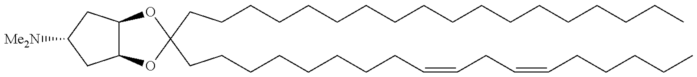

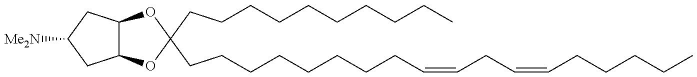

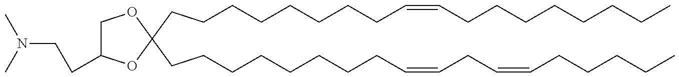 [SMILES: CCCCC/C=C\C/C=C\CCCCCCCCC1(CCCCCCCC/C=C\CCCCCCCC)OCC(CCN(C)C)O1]

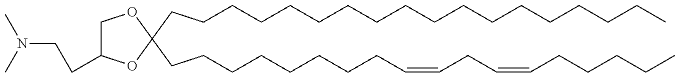 [SMILES: CCCCC/C=C\C/C=C\CCCCCCCCC1(CCCCCCCCCCCCCCCCCC)OCC(CCN(C)C)O1]

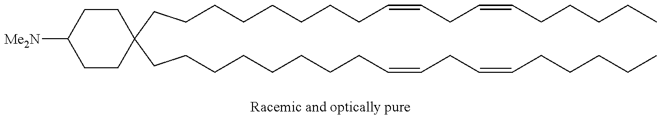 [SMILES: CCCCC/C=C\C/C=C\CCCCCCCCC1(CCCCCCCC/C=C\C/C=C\CCCCC)CCC(N(C)C)CC1]

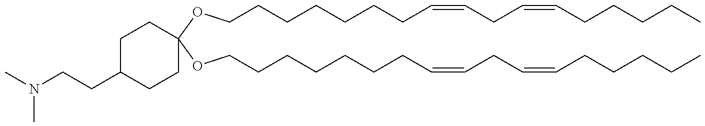 [SMILES: CCCCC/C=C\C/C=C\CCCCCCCOC1(OCCCCCCC/C=C\C/C=C\CCCCC)CCC(CCN(C)C)CC1]

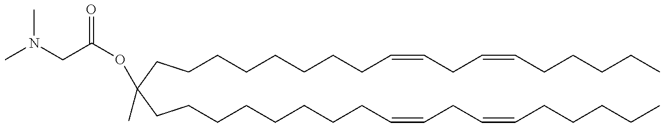 [SMILES: CCCCC/C=C\C/C=C\CCCCCCCCC(C)(CCCCCCCC/C=C\C/C=C\CCCCC)OC(=O)CN(C)C]

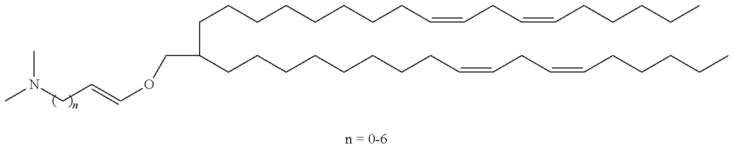

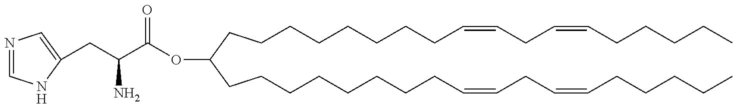 [SMILES: CCCCC/C=C\C/C=C\CCCCCCCCC(CCCCCCCC/C=C\C/C=C\CCCCC)OC(=O)[C@@H](N)Cc1cnc[nH]1]

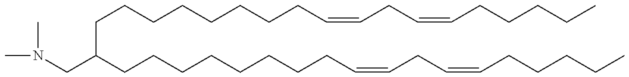 [SMILES: CCCCC/C=C\C/C=C\CCCCCCCCC(CCCCCCCC/C=C\C/C=C\CCCCC)CN(C)C]

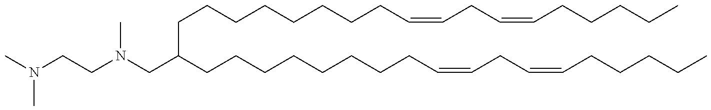 [SMILES: CCCCC/C=C\C/C=C\CCCCCCCCC(CCCCCCCC/C=C\C/C=C\CCCCC)CN(C)CCN(C)C]

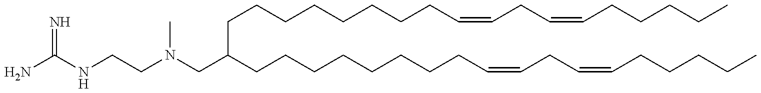 [SMILES: CCCCC/C=C\C/C=C\CCCCCCCCC(CCCCCCCC/C=C\C/C=C\CCCCC)CN(C)CCNC(=N)N]

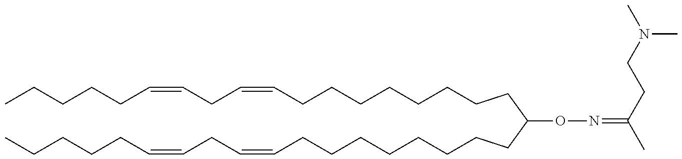 [SMILES: CCCCC/C=C\C/C=C\CCCCCCCCC(CCCCCCCC/C=C\C/C=C\CCCCC)ON=C(C)CCN(C)C]

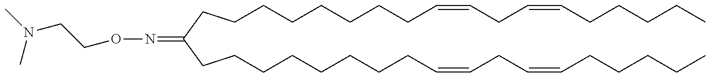 [SMILES: CCCCC/C=C\C/C=C\CCCCCCCCC(CCCCCCCC/C=C\C/C=C\CCCCC)=NOCCN(C)C]

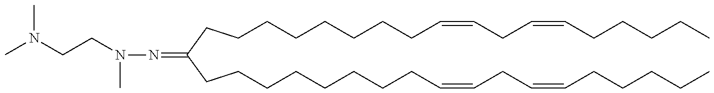 [SMILES: CCCCC/C=C\C/C=C\CCCCCCCCC(CCCCCCCC/C=C\C/C=C\CCCCC)=NN(C)CCN(C)C]

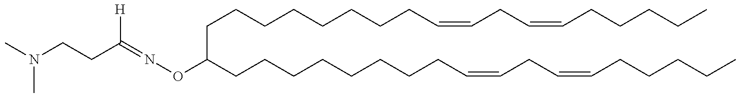 [SMILES: CCCCC/C=C\C/C=C\CCCCCCCCC(CCCCCCCC/C=C\C/C=C\CCCCC)ON=CCCN(C)C]

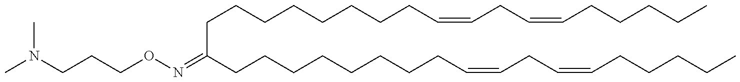 [SMILES: CCCCC/C=C\C/C=C\CCCCCCCCC(CCCCCCCC/C=C\C/C=C\CCCCC)=NOCCCN(C)C]

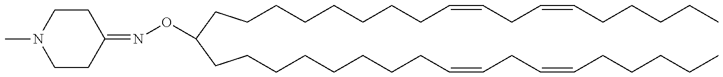 [SMILES: CCCCC/C=C\C/C=C\CCCCCCCCC(CCCCCCCC/C=C\C/C=C\CCCCC)ON=C1CCN(C)CC1]

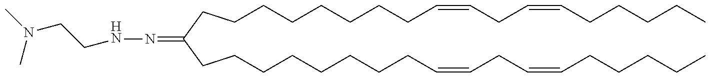 [SMILES: CCCCC/C=C\C/C=C\CCCCCCCCC(CCCCCCCC/C=C\C/C=C\CCCCC)=NNCCN(C)C]

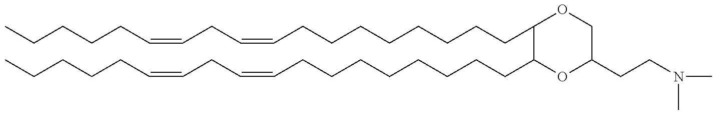 [SMILES: CCCCC/C=C\C/C=C\CCCCCCCCC1OCC(CCN(C)C)OC1CCCCCCCC/C=C\C/C=C\CCCCC]

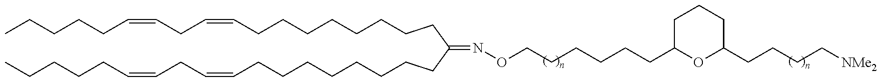

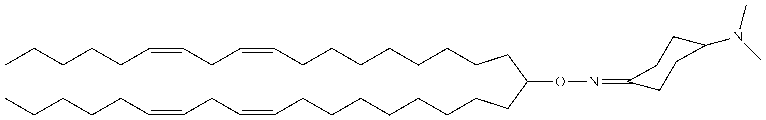 [SMILES: CCCCC/C=C\C/C=C\CCCCCCCCC(CCCCCCCC/C=C\C/C=C\CCCCC)ON=C1CCC(N(C)C)CC1]

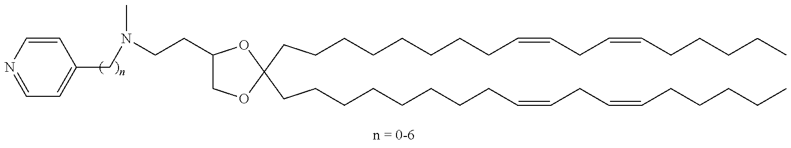

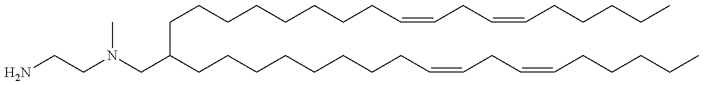 [SMILES: CCCCC/C=C\C/C=C\CCCCCCCCC(CCCCCCCC/C=C\C/C=C\CCCCC)CN(C)CCN]

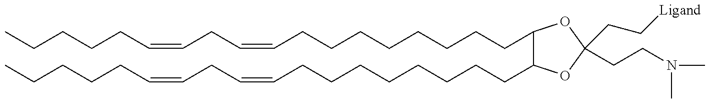

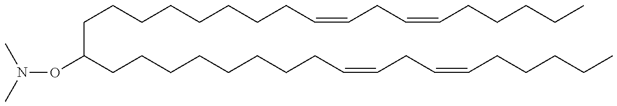 [SMILES: CCCCC/C=C\C/C=C\CCCCCCCCC(CCCCCCCC/C=C\C/C=C\CCCCC)ON(C)C]

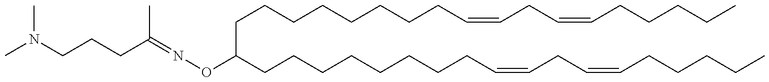 [SMILES: CCCCC/C=C\C/C=C\CCCCCCCCC(CCCCCCCC/C=C\C/C=C\CCCCC)ON=C(C)CCCN(C)C]

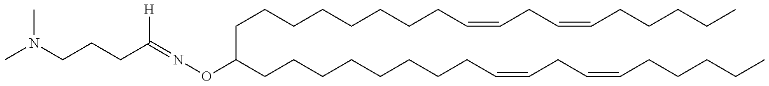 [SMILES: CCCCC/C=C\C/C=C\CCCCCCCCC(CCCCCCCC/C=C\C/C=C\CCCCC)ON=CCCCN(C)C]

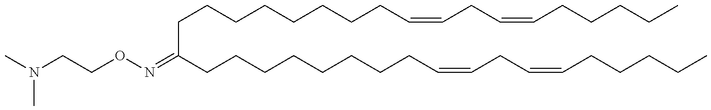 [SMILES: CCCCC/C=C\C/C=C\CCCCCCCCC(CCCCCCCC/C=C\C/C=C\CCCCC)=NOCCN(C)C]

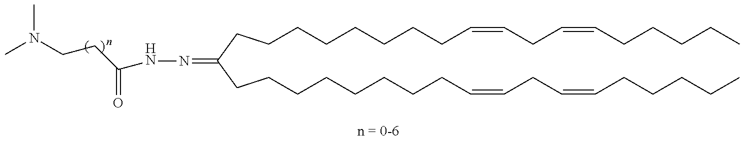

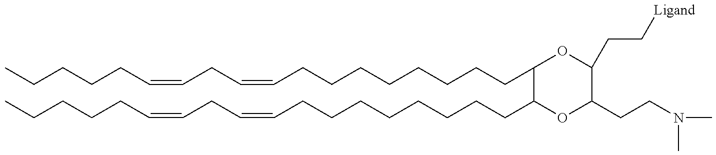

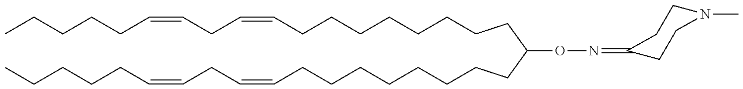 [SMILES: CCCCC/C=C\C/C=C\CCCCCCCCC(CCCCCCCC/C=C\C/C=C\CCCCC)ON=C1CCN(C)CC1]

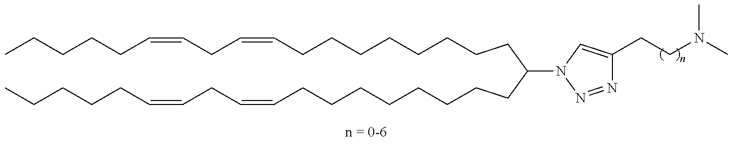

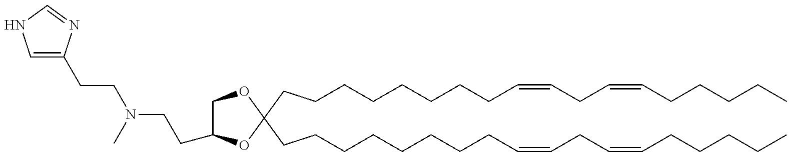

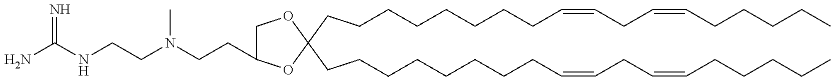 [SMILES: CCCCC/C=C\C/C=C\CCCCCCCCC1(CCCCCCCC/C=C\C/C=C\CCCCC)OCC(CCN(C)CCNC(=N)N)O1]

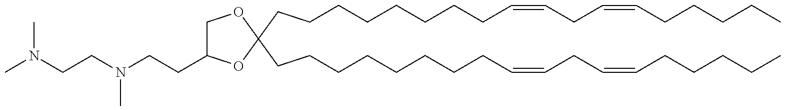 [SMILES: CCCCC/C=C\C/C=C\CCCCCCCCC1(CCCCCCCC/C=C\C/C=C\CCCCC)OCC(CCN(C)CCN(C)C)O1]

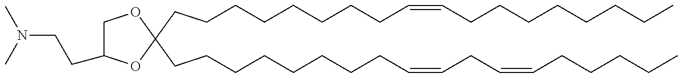 [SMILES: CCCCC/C=C\C/C=C\CCCCCCCCC1(CCCCCCCC/C=C\CCCCCCCC)OCC(CCN(C)C)O1]

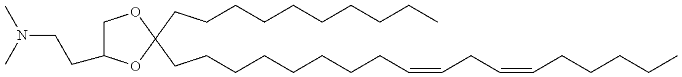 [SMILES: CCCCC/C=C\C/C=C\CCCCCCCCC1(CCCCCCCCCC)OCC(CCN(C)C)O1]

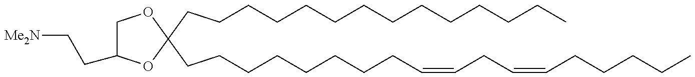 [SMILES: CCCCC/C=C\C/C=C\CCCCCCCCC1(CCCCCCCCCCCCCC)OCC(CCN(C)C)O1]

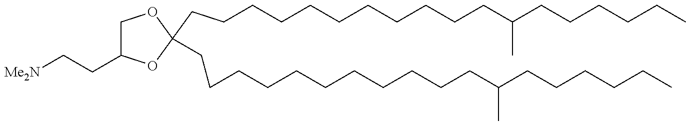 [SMILES: CCCCCCC(C)CCCCCCCCCCCC1(CCCCCCCCCCCC(C)CCCCCC)OCC(CCN(C)C)O1]

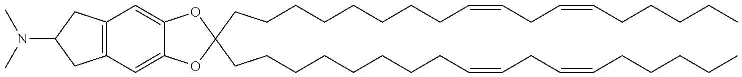 [SMILES: CCCCC/C=C\C/C=C\CCCCCCCCC1(CCCCCCCC/C=C\C/C=C\CCCCC)Oc2cc3c(cc2O1)CC(N(C)C)C3]

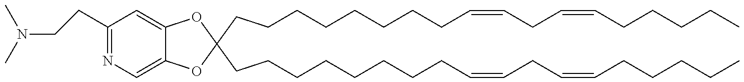 [SMILES: CCCCC/C=C\C/C=C\CCCCCCCCC1(CCCCCCCC/C=C\C/C=C\CCCCC)Oc2cnc(CCN(C)C)cc2O1]

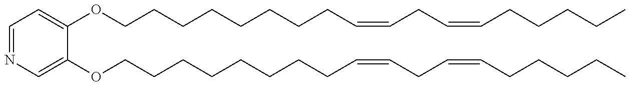 [SMILES: CCCCC/C=C\C/C=C\CCCCCCCCOc1ccncc1OCCCCCCCC/C=C\C/C=C\CCCCC]

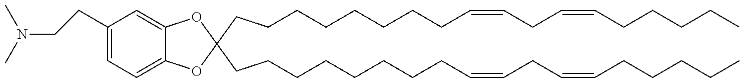 [SMILES: CCCCC/C=C\C/C=C\CCCCCCCCC1(CCCCCCCC/C=C\C/C=C\CCCCC)Oc2ccc(CCN(C)C)cc2O1]

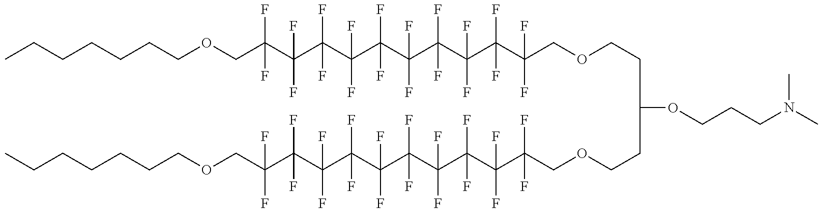 [SMILES: CCCCCCCOCC(F)(F)C(F)(F)C(F)(F)C(F)(F)C(F)(F)C(F)(F)C(F)(F)C(F)(F)C(F)(F)C(F)(F)COCCC(CCOCC(F)(F)C(F)(F)C(F)(F)C(F)(F)C(F)(F)C(F)(F)C(F)(F)C(F)(F)C(F)(F)C(F)(F)COCCCCCCC)OCCCN(C)C]

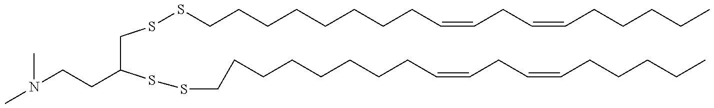 [SMILES: CCCCC/C=C\C/C=C\CCCCCCCCSSCC(CCN(C)C)SSCCCCCCCC/C=C\C/C=C\CCCCC]

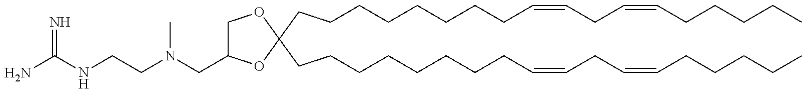 [SMILES: CCCCC/C=C\C/C=C\CCCCCCCCC1(CCCCCCCC/C=C\C/C=C\CCCCC)OCC(CN(C)CCNC(=N)N)O1]

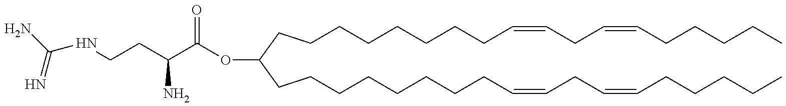 [SMILES: CCCCC/C=C\C/C=C\CCCCCCCCC(CCCCCCCC/C=C\C/C=C\CCCCC)OC(=O)[C@@H](N)CCNC(=N)N]

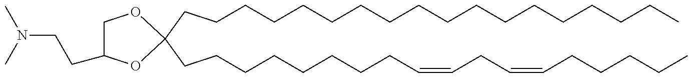 [SMILES: CCCCC/C=C\C/C=C\CCCCCCCCC1(CCCCCCCCCCCCCCCCCC)OCC(CCN(C)C)O1]

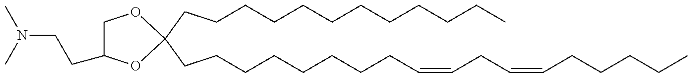 [SMILES: CCCCC/C=C\C/C=C\CCCCCCCCC1(CCCCCCCCCCCC)OCC(CCN(C)C)O1]

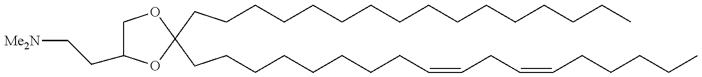 [SMILES: CCCCC/C=C\C/C=C\CCCCCCCCC1(CCCCCCCCCCCCCCCC)OCC(CCN(C)C)O1]

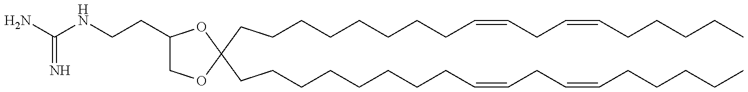 [SMILES: CCCCC/C=C\C/C=C\CCCCCCCCC1(CCCCCCCC/C=C\C/C=C\CCCCC)OCC(CCNC(=N)N)O1]

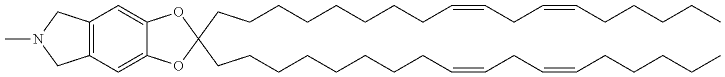 [SMILES: CCCCC/C=C\C/C=C\CCCCCCCCC1(CCCCCCCC/C=C\C/C=C\CCCCC)Oc2cc3c(cc2O1)CN(C)C3]

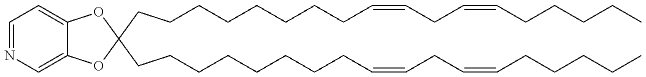 [SMILES: CCCCC/C=C\C/C=C\CCCCCCCCC1(CCCCCCCC/C=C\C/C=C\CCCCC)Oc2ccncc2O1]

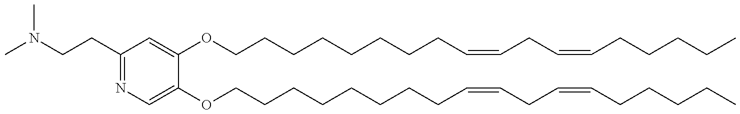 [SMILES: CCCCC/C=C\C/C=C\CCCCCCCCOc1cnc(CCN(C)C)cc1OCCCCCCCC/C=C\C/C=C\CCCCC]

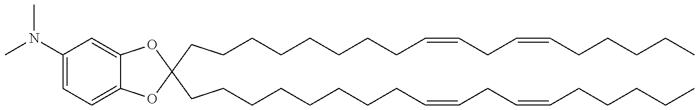 [SMILES: CCCCC/C=C\C/C=C\CCCCCCCCC1(CCCCCCCC/C=C\C/C=C\CCCCC)Oc2ccc(N(C)C)cc2O1]

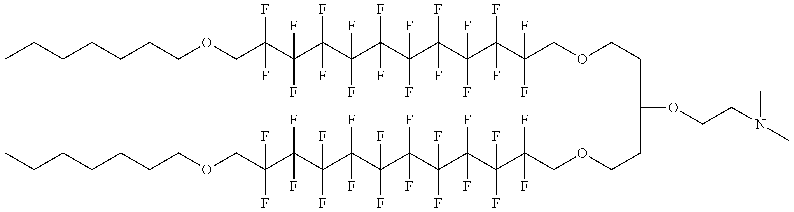 [SMILES: CCCCCCCOCC(F)(F)C(F)(F)C(F)(F)C(F)(F)C(F)(F)C(F)(F)C(F)(F)C(F)(F)C(F)(F)C(F)(F)COCCC(CCOCC(F)(F)C(F)(F)C(F)(F)C(F)(F)C(F)(F)C(F)(F)C(F)(F)C(F)(F)C(F)(F)C(F)(F)COCCCCCCC)OCCN(C)C]

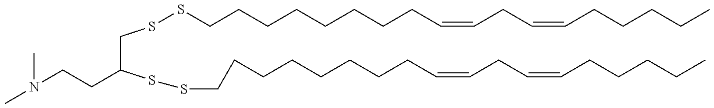 [SMILES: CCCCC/C=C\C/C=C\CCCCCCCCSSCC(CCN(C)C)SSCCCCCCCC/C=C\C/C=C\CCCCC]

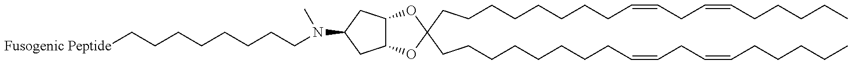

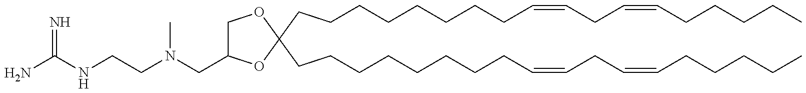 [SMILES: CCCCC/C=C\C/C=C\CCCCCCCCC1(CCCCCCCC/C=C\C/C=C\CCCCC)OCC(CN(C)CCNC(=N)N)O1]

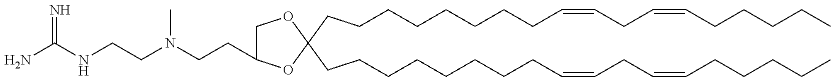 [SMILES: CCCCC/C=C\C/C=C\CCCCCCCCC1(CCCCCCCC/C=C\C/C=C\CCCCC)OCC(CCN(C)CCNC(=N)N)O1]

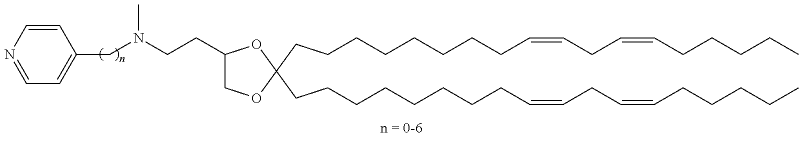

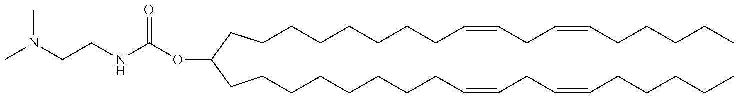 [SMILES: CCCCC/C=C\C/C=C\CCCCCCCCC(CCCCCCCC/C=C\C/C=C\CCCCC)OC(=O)NCCN(C)C]

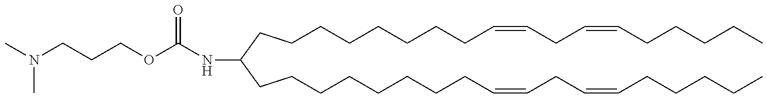 [SMILES: CCCCC/C=C\C/C=C\CCCCCCCCC(CCCCCCCC/C=C\C/C=C\CCCCC)NC(=O)OCCCN(C)C]

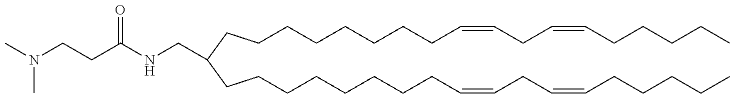 [SMILES: CCCCC/C=C\C/C=C\CCCCCCCCC(CCCCCCCC/C=C\C/C=C\CCCCC)CNC(=O)CCN(C)C]

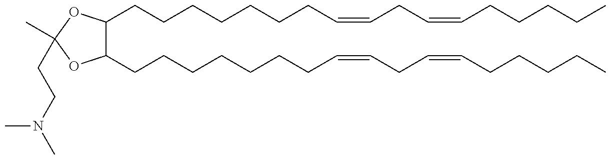 [SMILES: CCCCC/C=C\C/C=C\CCCCCCCC1OC(C)(CCN(C)C)OC1CCCCCCC/C=C\C/C=C\CCCCC]

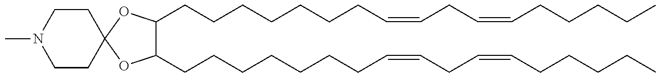 [SMILES: CCCCC/C=C\C/C=C\CCCCCCCC1OC2(CCN(C)CC2)OC1CCCCCCC/C=C\C/C=C\CCCCC]

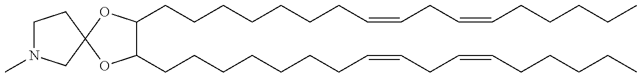 [SMILES: CCCCC/C=C\C/C=C\CCCCCCCC1OC2(CCN(C)C2)OC1CCCCCCC/C=C\C/C=C\CCCCC]

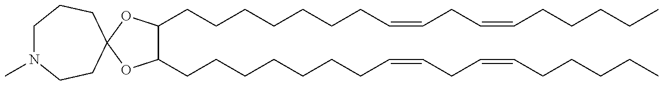 [SMILES: CCCCC/C=C\C/C=C\CCCCCCCC1OC2(CCCN(C)CC2)OC1CCCCCCC/C=C\C/C=C\CCCCC]

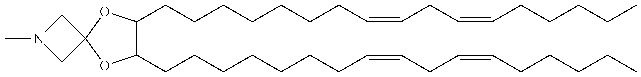 [SMILES: CCCCC/C=C\C/C=C\CCCCCCCC1OC2(CN(C)C2)OC1CCCCCCC/C=C\C/C=C\CCCCC]

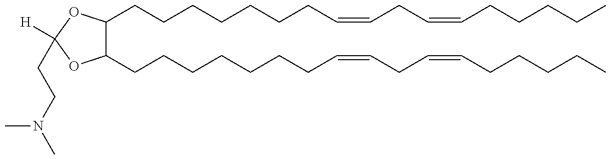 [SMILES: CCCCC/C=C\C/C=C\CCCCCCCC1OC(CCN(C)C)OC1CCCCCCC/C=C\C/C=C\CCCCC]

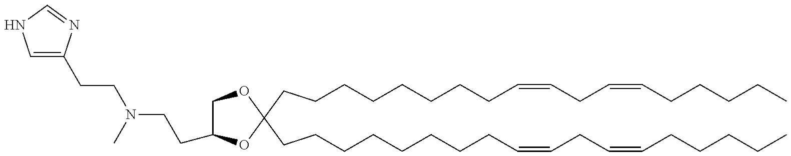

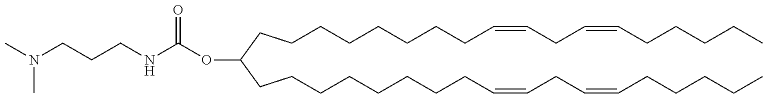 [SMILES: CCCCC/C=C\C/C=C\CCCCCCCCC(CCCCCCCC/C=C\C/C=C\CCCCC)OC(=O)NCCCN(C)C]

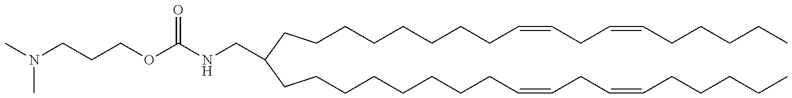 [SMILES: CCCCC/C=C\C/C=C\CCCCCCCCC(CCCCCCCC/C=C\C/C=C\CCCCC)CNC(=O)OCCCN(C)C]

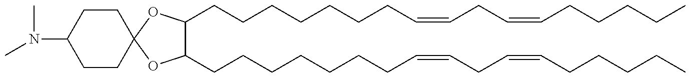 [SMILES: CCCCC/C=C\C/C=C\CCCCCCCC1OC2(CCC(N(C)C)CC2)OC1CCCCCCC/C=C\C/C=C\CCCCC]

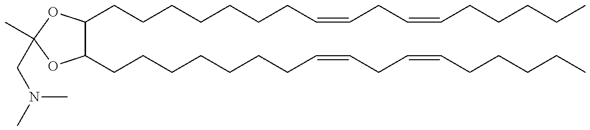 [SMILES: CCCCC/C=C\C/C=C\CCCCCCCC1OC(C)(CN(C)C)OC1CCCCCCC/C=C\C/C=C\CCCCC]

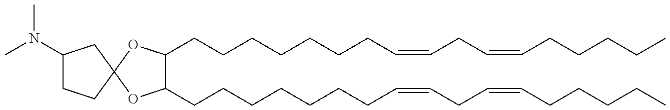 [SMILES: CCCCC/C=C\C/C=C\CCCCCCCC1OC2(CCC(N(C)C)C2)OC1CCCCCCC/C=C\C/C=C\CCCCC]

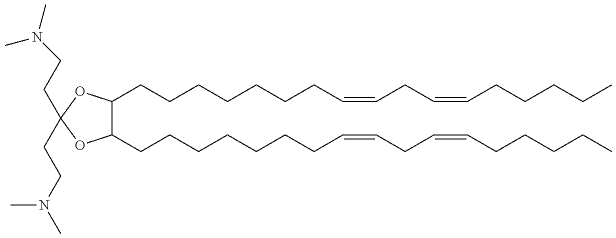 [SMILES: CCCCC/C=C\C/C=C\CCCCCCCC1OC(CCN(C)C)(CCN(C)C)OC1CCCCCCC/C=C\C/C=C\CCCCC]

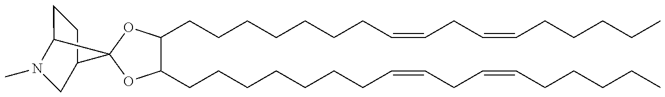 [SMILES: CCCCC/C=C\C/C=C\CCCCCCCC1OC2(OC1CCCCCCC/C=C\C/C=C\CCCCC)C1CCC2N(C)C1]

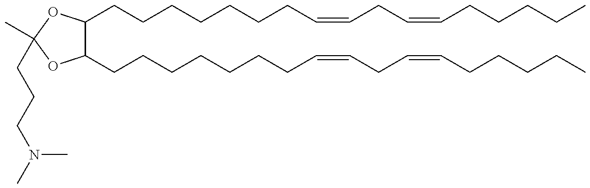 [SMILES: CCCCC/C=C\C/C=C\CCCCCCCC1OC(C)(CCCN(C)C)OC1CCCCCCC/C=C\C/C=C\CCCCC]

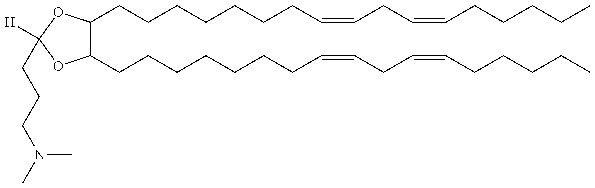 [SMILES: CCCCC/C=C\C/C=C\CCCCCCCC1OC(CCCN(C)C)OC1CCCCCCC/C=C\C/C=C\CCCCC]

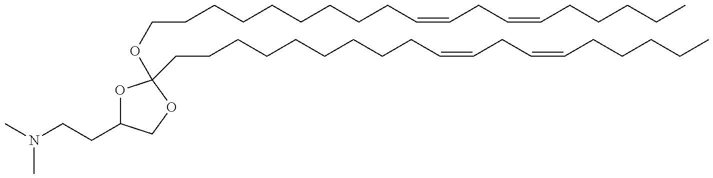 [SMILES: CCCCC/C=C\C/C=C\CCCCCCCCCOC1(CCCCCCCCC/C=C\C/C=C\CCCCC)OCC(CCN(C)C)O1]

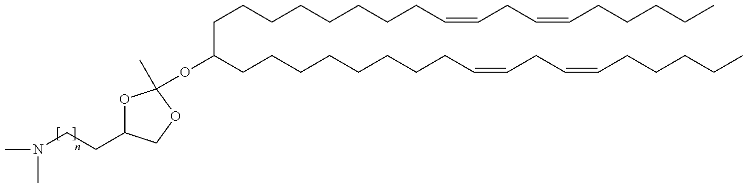

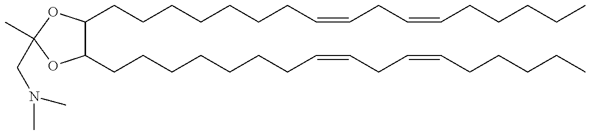 [SMILES: CCCCC/C=C\C/C=C\CCCCCCCC1OC(C)(CN(C)C)OC1CCCCCCC/C=C\C/C=C\CCCCC]

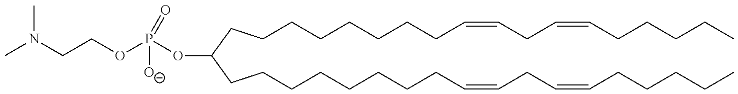 [SMILES: CCCCC/C=C\C/C=C\CCCCCCCCC(CCCCCCCC/C=C\C/C=C\CCCCC)OP(=O)([O-])OCCN(C)C]

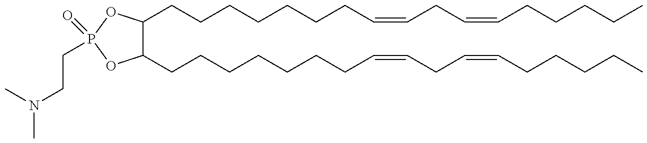 [SMILES: CCCCC/C=C\C/C=C\CCCCCCCC1OP(=O)(CCN(C)C)OC1CCCCCCC/C=C\C/C=C\CCCCC]

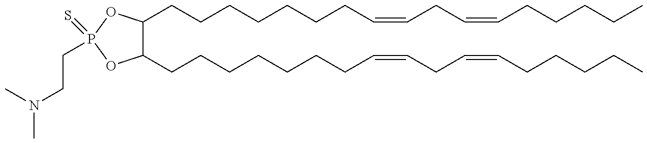 [SMILES: CCCCC/C=C\C/C=C\CCCCCCCC1OP(=S)(CCN(C)C)OC1CCCCCCC/C=C\C/C=C\CCCCC]

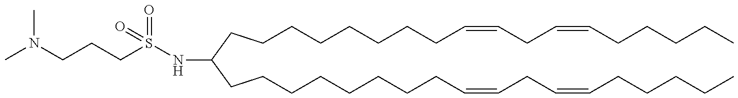 [SMILES: CCCCC/C=C\C/C=C\CCCCCCCCC(CCCCCCCC/C=C\C/C=C\CCCCC)NS(=O)(=O)CCCN(C)C]

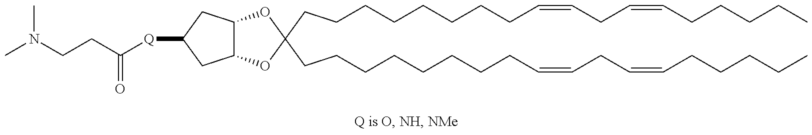

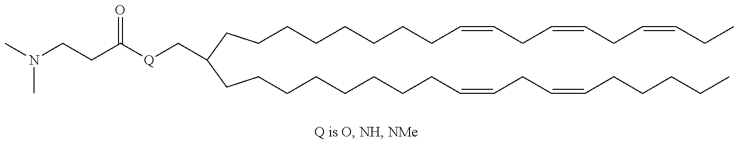

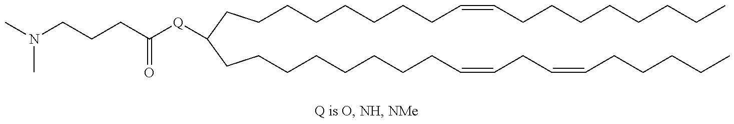

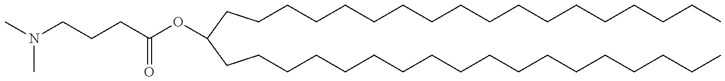 [SMILES: CCCCCCCCCCCCCCCCCCC(CCCCCCCCCCCCCCCCCC)OC(=O)CCCN(C)C]

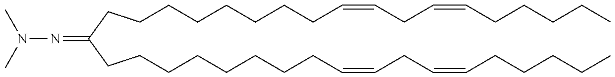 [SMILES: CCCCC/C=C\C/C=C\CCCCCCCCC(CCCCCCCC/C=C\C/C=C\CCCCC)=NN(C)C]

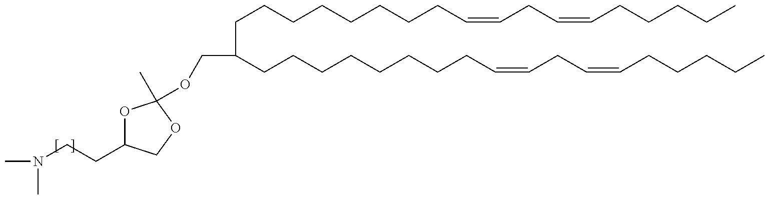

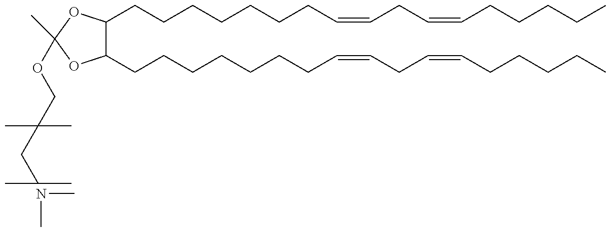

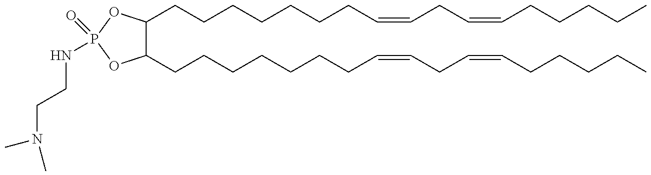 [SMILES: CCCCC/C=C\C/C=C\CCCCCCCC1OP(=O)(NCCN(C)C)OC1CCCCCCC/C=C\C/C=C\CCCCC]

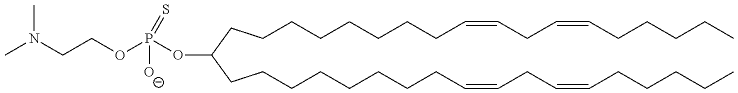 [SMILES: CCCCC/C=C\C/C=C\CCCCCCCCC(CCCCCCCC/C=C\C/C=C\CCCCC)OP([O-])(=S)OCCN(C)C]

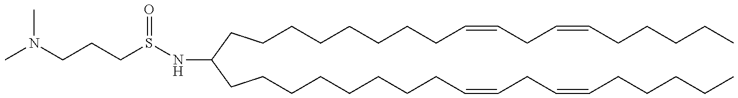 [SMILES: CCCCC/C=C\C/C=C\CCCCCCCCC(CCCCCCCC/C=C\C/C=C\CCCCC)NS(=O)CCCN(C)C]

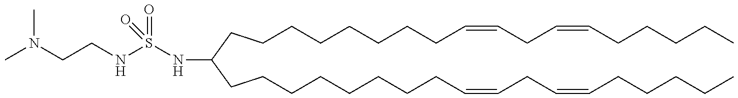 [SMILES: CCCCC/C=C\C/C=C\CCCCCCCCC(CCCCCCCC/C=C\C/C=C\CCCCC)NS(=O)(=O)NCCN(C)C]

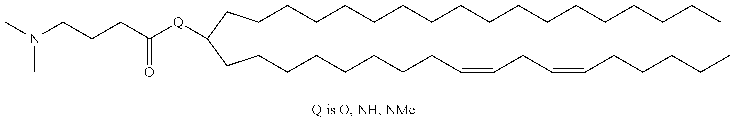

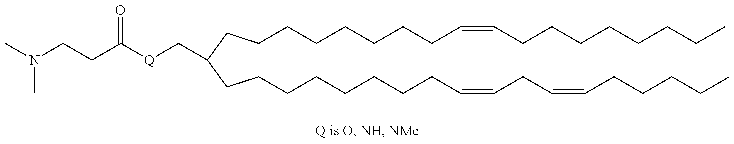

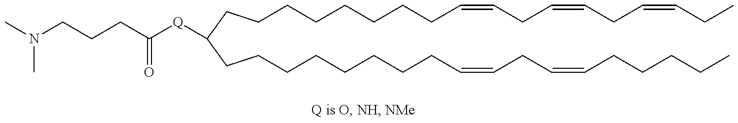

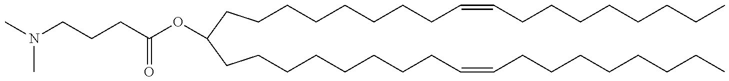 [SMILES: CCCCCCCC/C=C\CCCCCCCCC(CCCCCCCC/C=C\CCCCCCCC)OC(=O)CCCN(C)C]

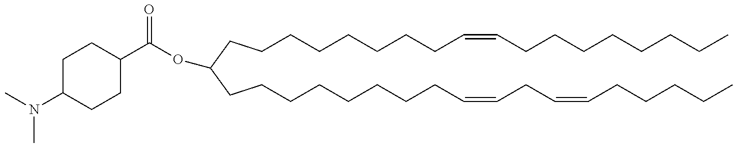 [SMILES: CCCCC/C=C\C/C=C\CCCCCCCCC(CCCCCCCC/C=C\CCCCCCCC)OC(=O)C1CCC(N(C)C)CC1]

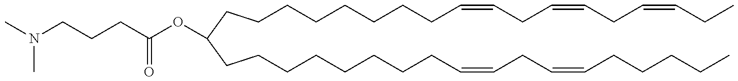 [SMILES: CC/C=C\C/C=C\C/C=C\CCCCCCCCC(CCCCCCCC/C=C\C/C=C\CCCCC)OC(=O)CCCN(C)C]

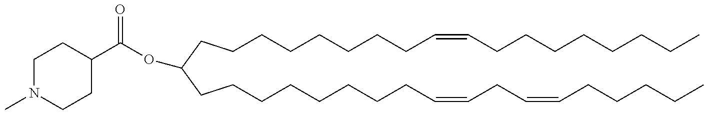 [SMILES: CCCCC/C=C\C/C=C\CCCCCCCCC(CCCCCCCC/C=C\CCCCCCCC)OC(=O)C1CCN(C)CC1]

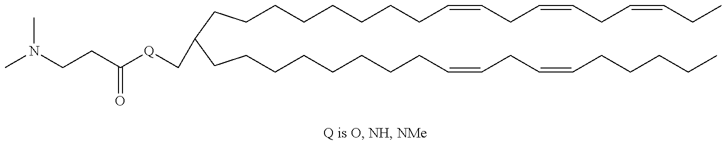

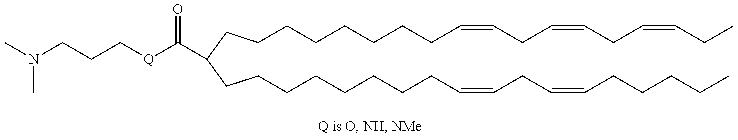

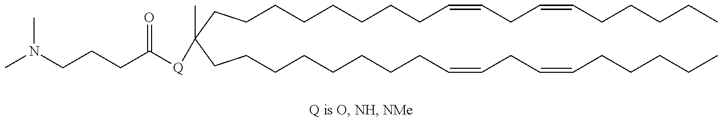

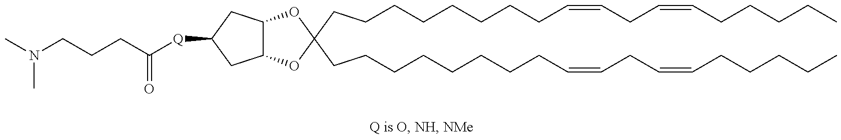

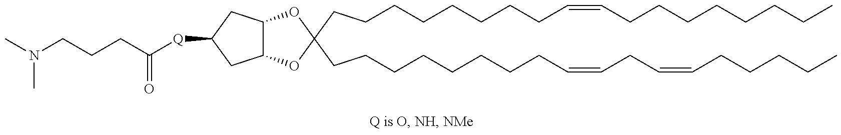

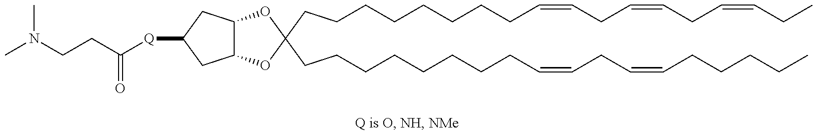

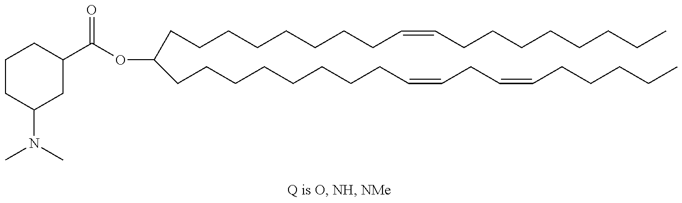 [SMILES: CCCCC/C=C\C/C=C\CCCCCCCCC(CCCCCCCC/C=C\CCCCCCCC)OC(=O)C1CCCC(N(C)C)C1]

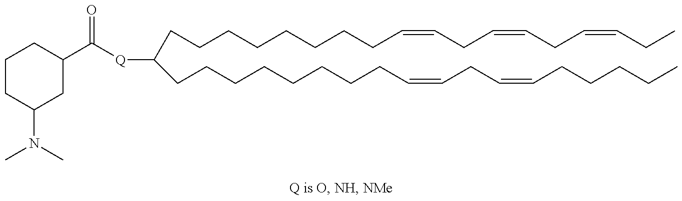

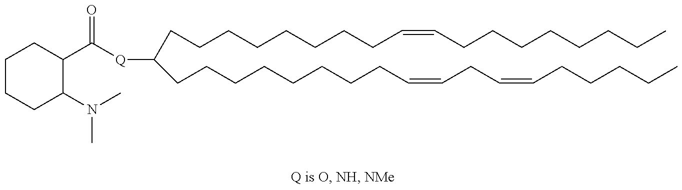

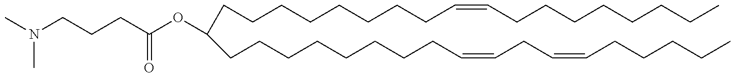 [SMILES: CCCCC/C=C\C/C=C\CCCCCCCCC(CCCCCCCC/C=C\CCCCCCCC)OC(=O)CCCN(C)C]

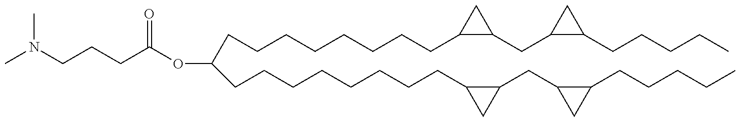 [SMILES: CCCCCC1CC1CC1CC1CCCCCCCCC(CCCCCCCCC1CC1CC1CC1CCCCC)OC(=O)CCCN(C)C]

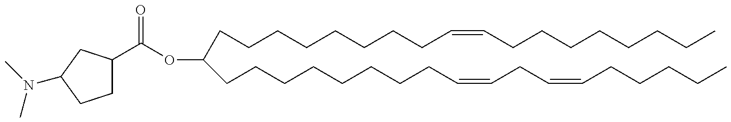 [SMILES: CCCCC/C=C\C/C=C\CCCCCCCCC(CCCCCCCC/C=C\CCCCCCCC)OC(=O)C1CCC(N(C)C)C1]

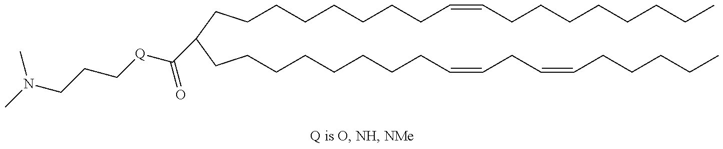

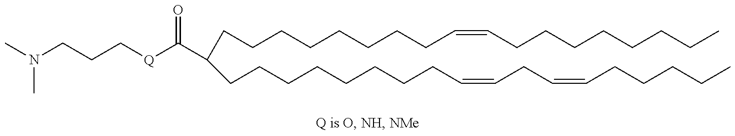

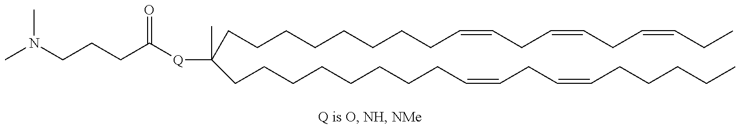

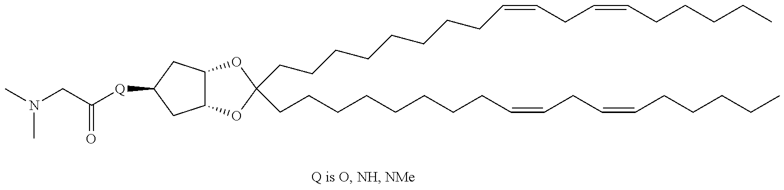

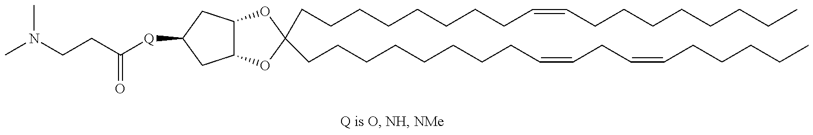

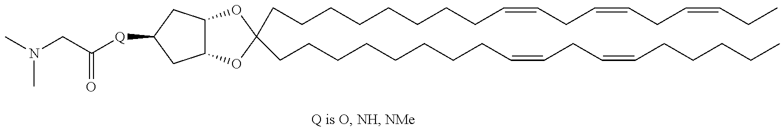

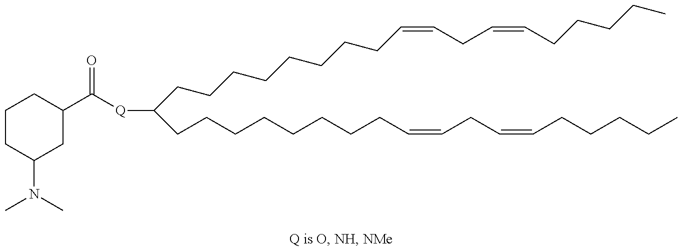

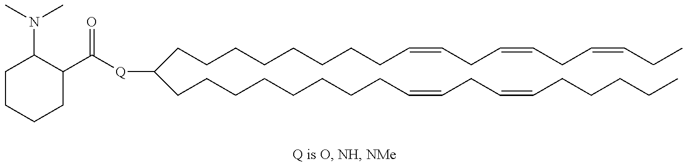

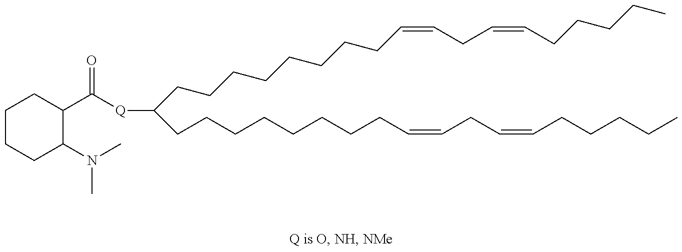

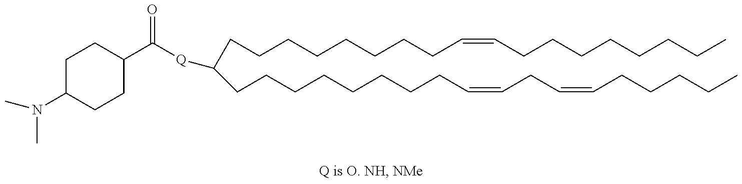

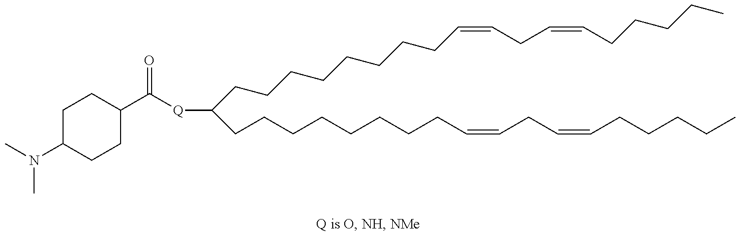

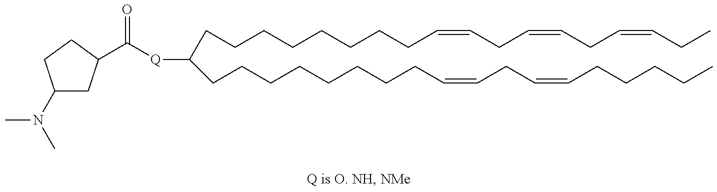

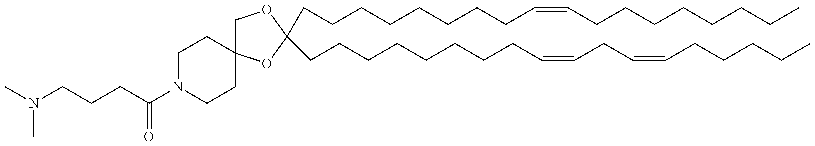 [SMILES: CCCCC/C=C\C/C=C\CCCCCCCCC1(CCCCCCCC/C=C\CCCCCCCC)OCC2(CCN(C(=O)CCCN(C)C)CC2)O1]

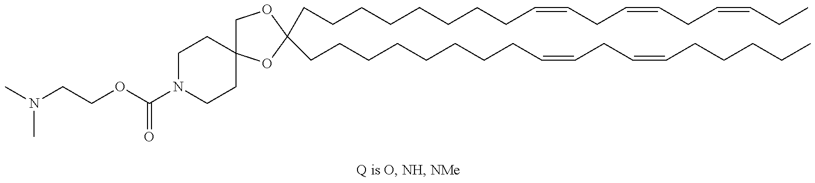 [SMILES: CC/C=C\C/C=C\C/C=C\CCCCCCCCC1(CCCCCCCC/C=C\C/C=C\CCCCC)OCC2(CCN(C(=O)OCCN(C)C)CC2)O1]

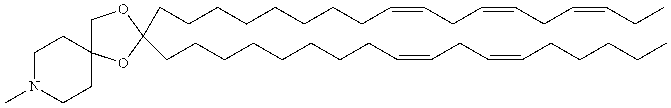 [SMILES: CC/C=C\C/C=C\C/C=C\CCCCCCCCC1(CCCCCCCC/C=C\C/C=C\CCCCC)OCC2(CCN(C)CC2)O1]

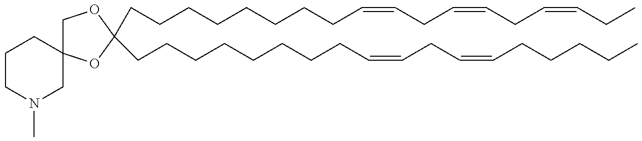 [SMILES: CC/C=C\C/C=C\C/C=C\CCCCCCCCC1(CCCCCCCC/C=C\C/C=C\CCCCC)OCC2(CCCN(C)C2)O1]

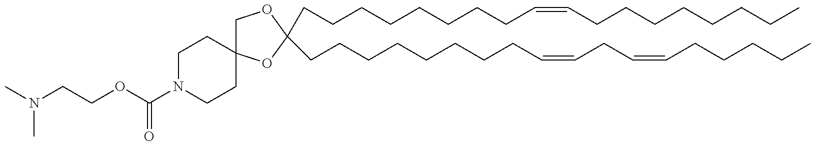 [SMILES: CCCCC/C=C\C/C=C\CCCCCCCCC1(CCCCCCCC/C=C\CCCCCCCC)OCC2(CCN(C(=O)OCCN(C)C)CC2)O1]

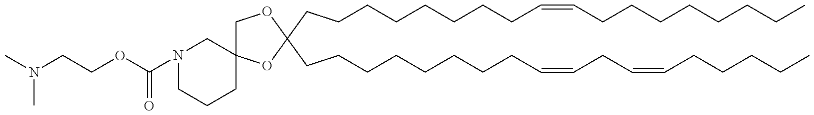 [SMILES: CCCCC/C=C\C/C=C\CCCCCCCCC1(CCCCCCCC/C=C\CCCCCCCC)OCC2(CCCN(C(=O)OCCN(C)C)C2)O1]

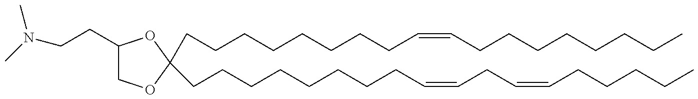 [SMILES: CCCCC/C=C\C/C=C\CCCCCCCCC1(CCCCCCCC/C=C\CCCCCCCC)OCC(CCN(C)C)O1]

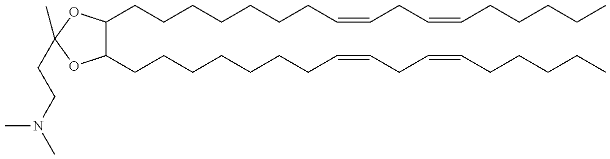 [SMILES: CCCCC/C=C\C/C=C\CCCCCCCC1OC(C)(CCN(C)C)OC1CCCCCCC/C=C\C/C=C\CCCCC]

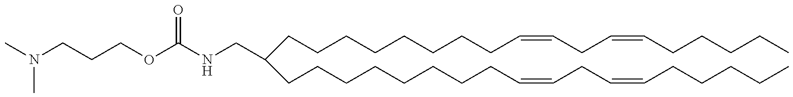 [SMILES: CCCCC/C=C\C/C=C\CCCCCCCCC(CCCCCCCC/C=C\C/C=C\CCCCC)CNC(=O)OCCCN(C)C]

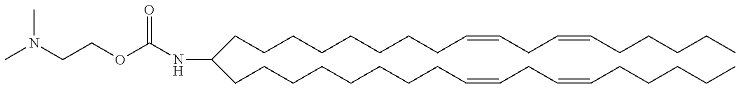 [SMILES: CCCCC/C=C\C/C=C\CCCCCCCCC(CCCCCCCC/C=C\C/C=C\CCCCC)NC(=O)OCCN(C)C]

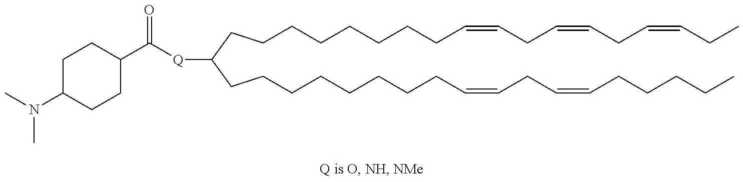

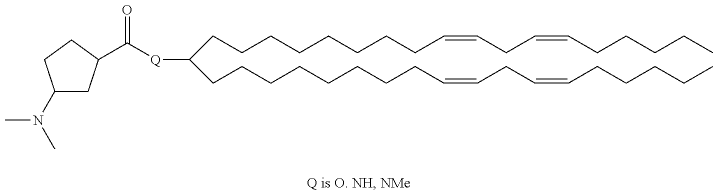

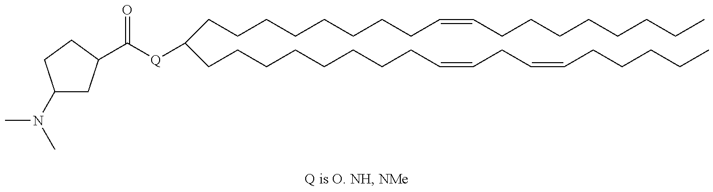

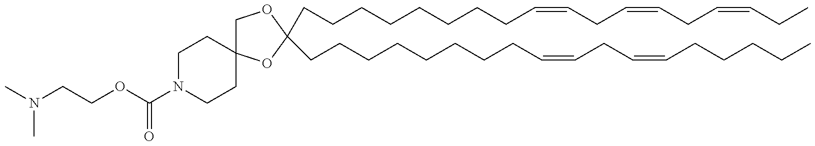 [SMILES: CC/C=C\C/C=C\C/C=C\CCCCCCCCC1(CCCCCCCC/C=C\C/C=C\CCCCC)OCC2(CCN(C(=O)OCCN(C)C)CC2)O1]

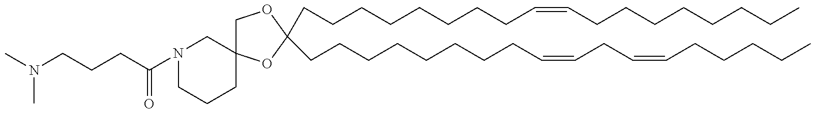 [SMILES: CCCCC/C=C\C/C=C\CCCCCCCCC1(CCCCCCCC/C=C\CCCCCCCC)OCC2(CCCN(C(=O)CCCN(C)C)C2)O1]

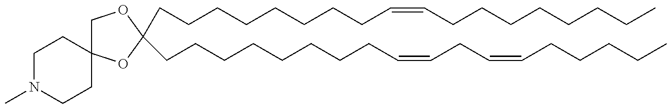 [SMILES: CCCCC/C=C\C/C=C\CCCCCCCCC1(CCCCCCCC/C=C\CCCCCCCC)OCC2(CCN(C)CC2)O1]

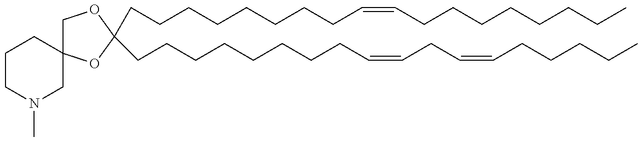 [SMILES: CCCCC/C=C\C/C=C\CCCCCCCCC1(CCCCCCCC/C=C\CCCCCCCC)OCC2(CCCN(C)C2)O1]

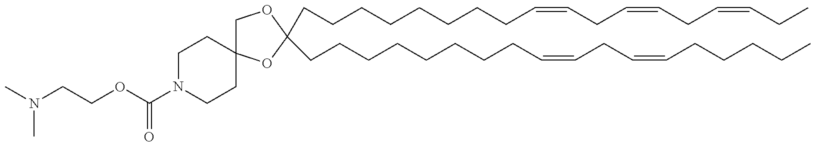 [SMILES: CC/C=C\C/C=C\C/C=C\CCCCCCCCC1(CCCCCCCC/C=C\C/C=C\CCCCC)OCC2(CCN(C(=O)OCCN(C)C)CC2)O1]

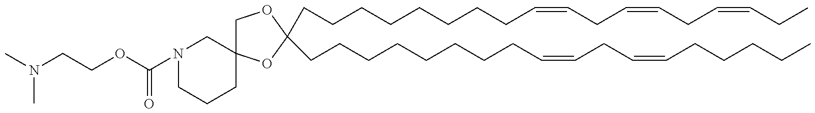 [SMILES: CC/C=C\C/C=C\C/C=C\CCCCCCCCC1(CCCCCCCC/C=C\C/C=C\CCCCC)OCC2(CCCN(C(=O)OCCN(C)C)C2)O1]

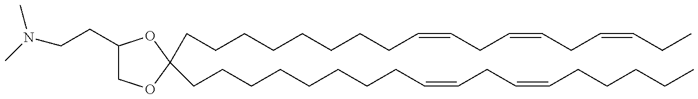 [SMILES: CC/C=C\C/C=C\C/C=C\CCCCCCCCC1(CCCCCCCC/C=C\C/C=C\CCCCC)OCC(CCN(C)C)O1]

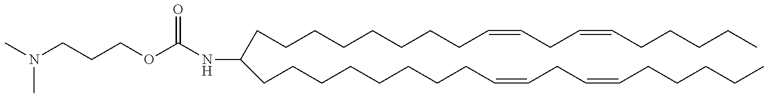 [SMILES: CCCCC/C=C\C/C=C\CCCCCCCCC(CCCCCCCC/C=C\C/C=C\CCCCC)NC(=O)OCCCN(C)C]

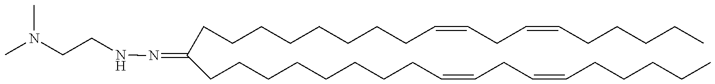 [SMILES: CCCCC/C=C\C/C=C\CCCCCCCCC(CCCCCCCC/C=C\C/C=C\CCCCC)=NNCCN(C)C]

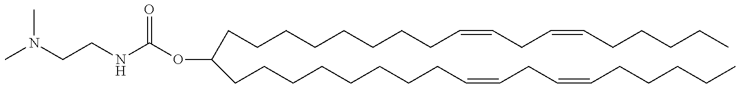 [SMILES: CCCCC/C=C\C/C=C\CCCCCCCCC(CCCCCCCC/C=C\C/C=C\CCCCC)OC(=O)NCCN(C)C]

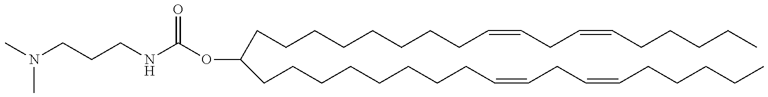 [SMILES: CCCCC/C=C\C/C=C\CCCCCCCCC(CCCCCCCC/C=C\C/C=C\CCCCC)OC(=O)NCCCN(C)C]

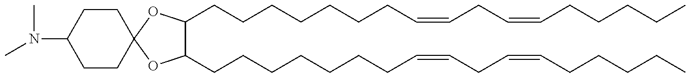 [SMILES: CCCCC/C=C\C/C=C\CCCCCCCC1OC2(CCC(N(C)C)CC2)OC1CCCCCCC/C=C\C/C=C\CCCCC]

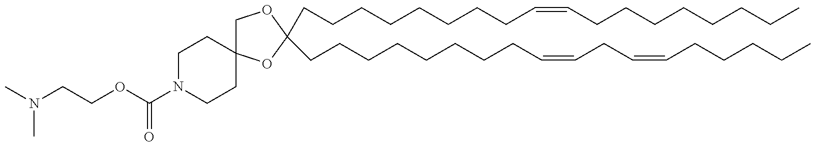 [SMILES: CCCCC/C=C\C/C=C\CCCCCCCCC1(CCCCCCCC/C=C\CCCCCCCC)OCC2(CCN(C(=O)OCCN(C)C)CC2)O1]

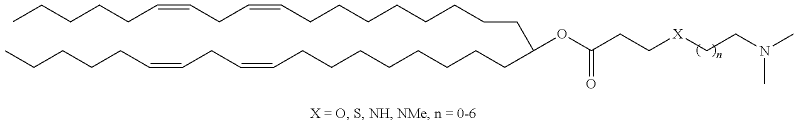

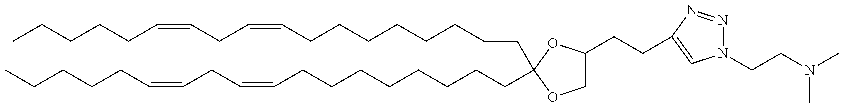 [SMILES: CCCCC/C=C\C/C=C\CCCCCCCCC1(CCCCCCCC/C=C\C/C=C\CCCCC)OCC(CCc2cn(CCN(C)C)nn2)O1]

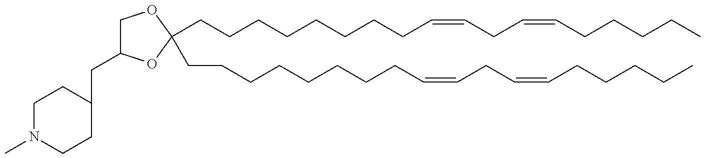 [SMILES: CCCCC/C=C\C/C=C\CCCCCCCCC1(CCCCCCCC/C=C\C/C=C\CCCCC)OCC(CC2CCN(C)CC2)O1]

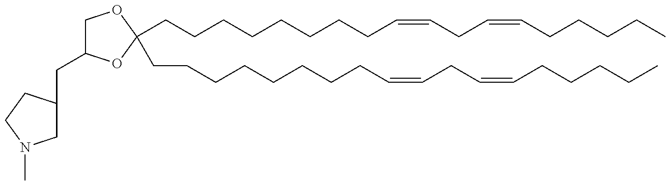 [SMILES: CCCCC/C=C\C/C=C\CCCCCCCCC1(CCCCCCCC/C=C\C/C=C\CCCCC)OCC(CC2CCN(C)C2)O1]

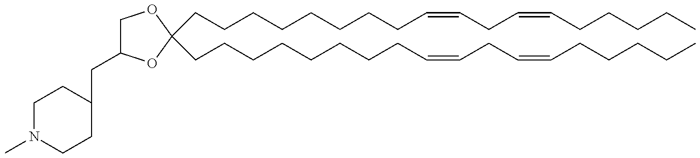 [SMILES: CCCCC/C=C\C/C=C\CCCCCCCCC1(CCCCCCCC/C=C\C/C=C\CCCCC)OCC(CC2CCN(C)CC2)O1]

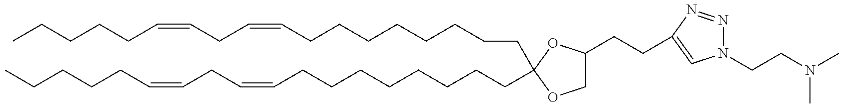 [SMILES: CCCCC/C=C\C/C=C\CCCCCCCCC1(CCCCCCCC/C=C\C/C=C\CCCCC)OCC(CCc2cn(CCN(C)C)nn2)O1]

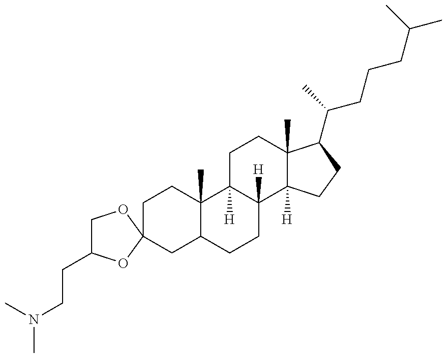

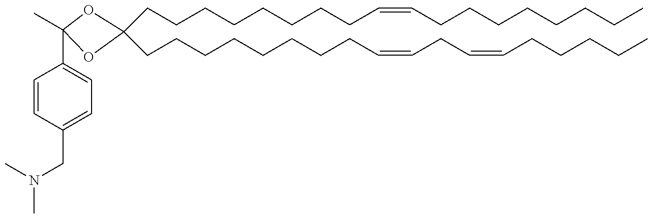 [SMILES: CCCCC/C=C\C/C=C\CCCCCCCCC1(CCCCCCCC/C=C\CCCCCCCC)OC(C)(c2ccc(CN(C)C)cc2)O1]

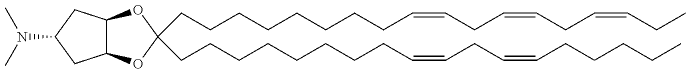

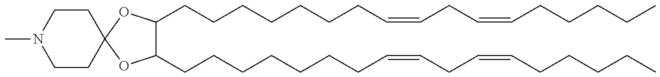 [SMILES: CCCCC/C=C\C/C=C\CCCCCCCC1OC2(CCN(C)CC2)OC1CCCCCCC/C=C\C/C=C\CCCCC]

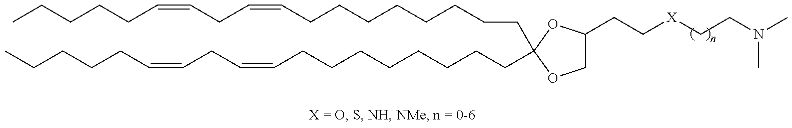

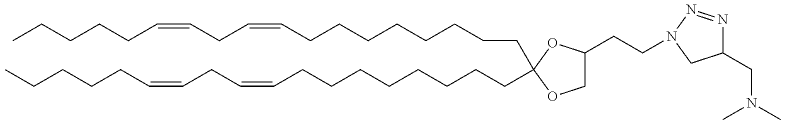 [SMILES: CCCCC/C=C\C/C=C\CCCCCCCCC1(CCCCCCCC/C=C\C/C=C\CCCCC)OCC(CCN2CC(CN(C)C)N=N2)O1]

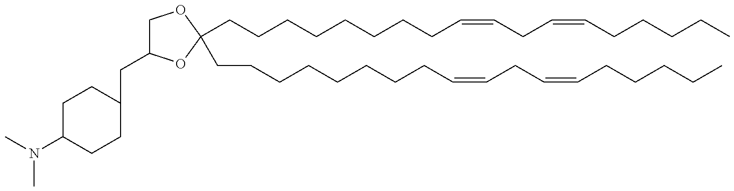 [SMILES: CCCCC/C=C\C/C=C\CCCCCCCCC1(CCCCCCCC/C=C\C/C=C\CCCCC)OCC(CC2CCC(N(C)C)CC2)O1]

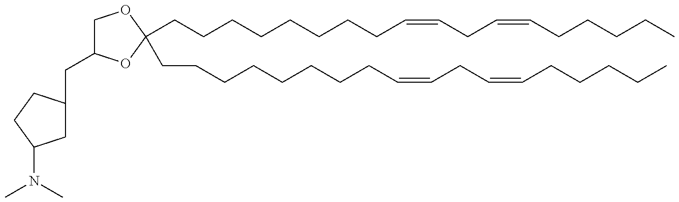 [SMILES: CCCCC/C=C\C/C=C\CCCCCCCCC1(CCCCCCCC/C=C\C/C=C\CCCCC)OCC(CC2CCC(N(C)C)C2)O1]

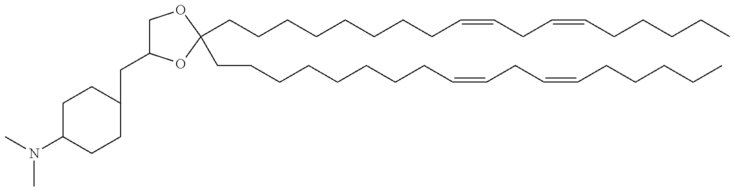 [SMILES: CCCCC/C=C\C/C=C\CCCCCCCCC1(CCCCCCCC/C=C\C/C=C\CCCCC)OCC(CC2CCC(N(C)C)CC2)O1]

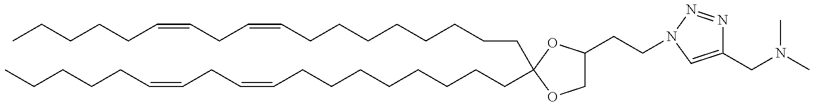 [SMILES: CCCCC/C=C\C/C=C\CCCCCCCCC1(CCCCCCCC/C=C\C/C=C\CCCCC)OCC(CCn2cc(CN(C)C)nn2)O1]

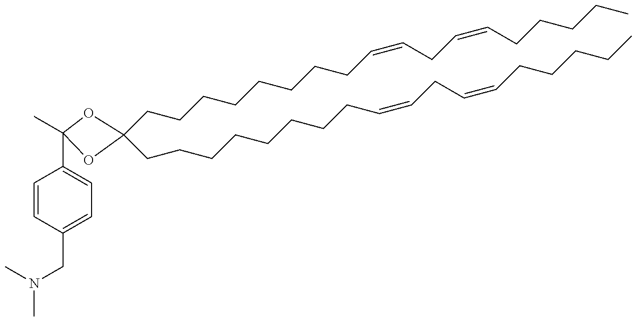 [SMILES: CCCCC/C=C\C/C=C\CCCCCCCCC1(CCCCCCCC/C=C\C/C=C\CCCCC)OC(C)(c2ccc(CN(C)C)cc2)O1]

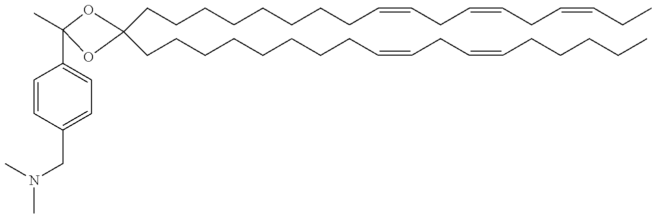 [SMILES: CC/C=C\C/C=C\C/C=C\CCCCCCCCC1(CCCCCCCC/C=C\C/C=C\CCCCC)OC(C)(c2ccc(CN(C)C)cc2)O1]

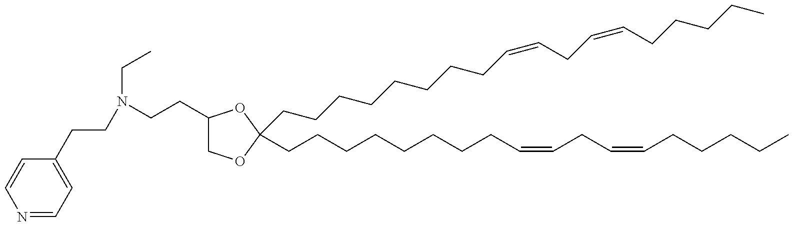 [SMILES: CCCCC/C=C\C/C=C\CCCCCCCCC1(CCCCCCCC/C=C\C/C=C\CCCCC)OCC(CCN(CC)CCc2ccncc2)O1]

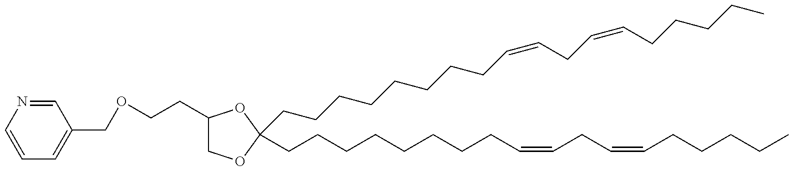 [SMILES: CCCCC/C=C\C/C=C\CCCCCCCCC1(CCCCCCCC/C=C\C/C=C\CCCCC)OCC(CCOCc2cccnc2)O1]

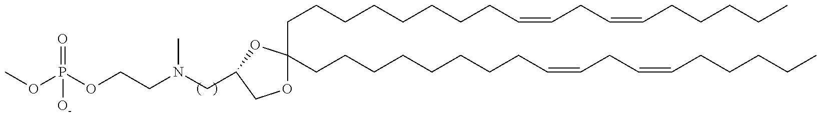

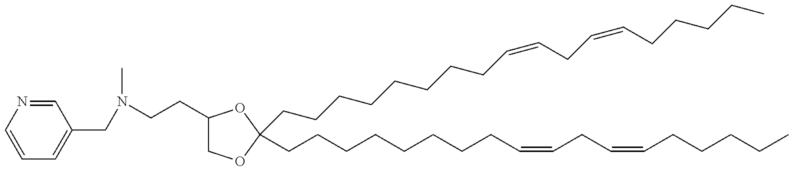 [SMILES: CCCCC/C=C\C/C=C\CCCCCCCCC1(CCCCCCCC/C=C\C/C=C\CCCCC)OCC(CCN(C)Cc2cccnc2)O1]

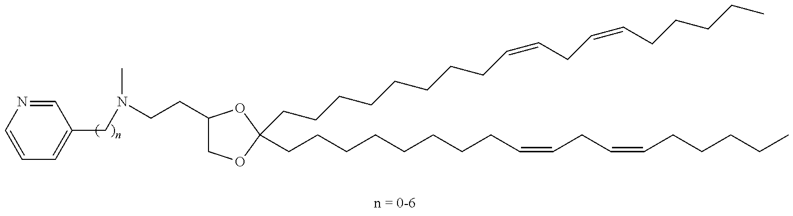

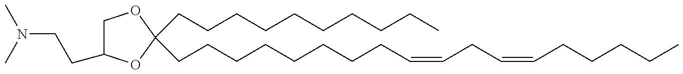 [SMILES: CCCCC/C=C\C/C=C\CCCCCCCCC1(CCCCCCCCCC)OCC(CCN(C)C)O1]

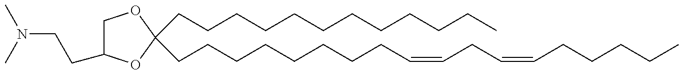 [SMILES: CCCCC/C=C\C/C=C\CCCCCCCCC1(CCCCCCCCCCCC)OCC(CCN(C)C)O1]

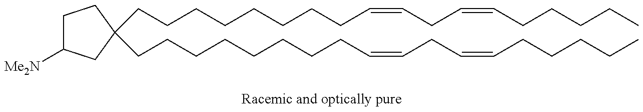 [SMILES: CCCCC/C=C\C/C=C\CCCCCCCCC1(CCCCCCCC/C=C\C/C=C\CCCCC)CCC(N(C)C)C1]

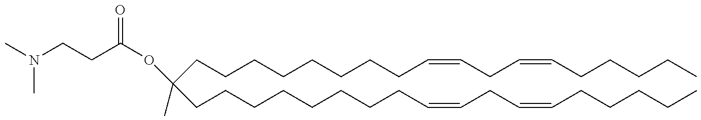 [SMILES: CCCCC/C=C\C/C=C\CCCCCCCCC(C)(CCCCCCCC/C=C\C/C=C\CCCCC)OC(=O)CCN(C)C]

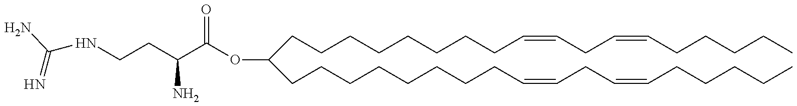 [SMILES: CCCCC/C=C\C/C=C\CCCCCCCCC(CCCCCCCC/C=C\C/C=C\CCCCC)OC(=O)[C@@H](N)CCNC(=N)N]

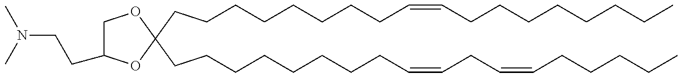 [SMILES: CCCCC/C=C\C/C=C\CCCCCCCCC1(CCCCCCCC/C=C\CCCCCCCC)OCC(CCN(C)C)O1]

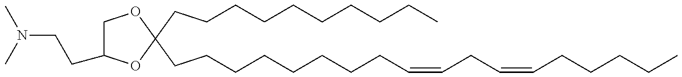 [SMILES: CCCCC/C=C\C/C=C\CCCCCCCCC1(CCCCCCCCCC)OCC(CCN(C)C)O1]

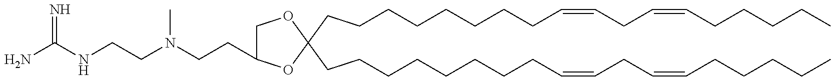 [SMILES: CCCCC/C=C\C/C=C\CCCCCCCCC1(CCCCCCCC/C=C\C/C=C\CCCCC)OCC(CCN(C)CCNC(=N)N)O1]

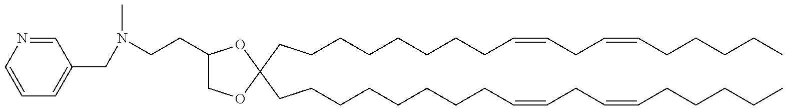 [SMILES: CCCCC/C=C\C/C=C\CCCCCCCCC1(CCCCCCCC/C=C\C/C=C\CCCCC)OCC(CCN(C)Cc2cccnc2)O1]

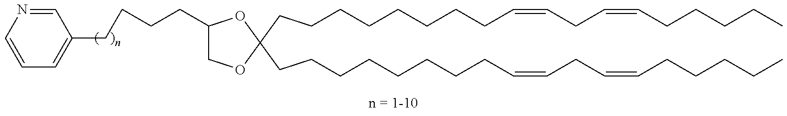

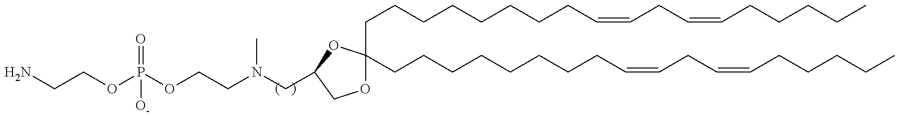

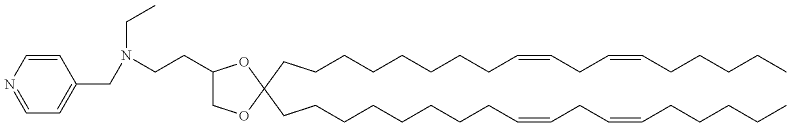 [SMILES: CCCCC/C=C\C/C=C\CCCCCCCCC1(CCCCCCCC/C=C\C/C=C\CCCCC)OCC(CCN(CC)Cc2ccncc2)O1]

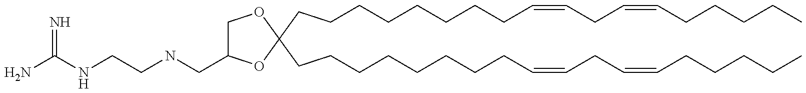 [SMILES: CCCCC/C=C\C/C=C\CCCCCCCCC1(CCCCCCCC/C=C\C/C=C\CCCCC)OCC(CNCCNC(=N)N)O1]

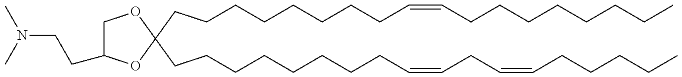 [SMILES: CCCCC/C=C\C/C=C\CCCCCCCCC1(CCCCCCCC/C=C\CCCCCCCC)OCC(CCN(C)C)O1]

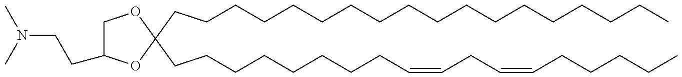 [SMILES: CCCCC/C=C\C/C=C\CCCCCCCCC1(CCCCCCCCCCCCCCCCCC)OCC(CCN(C)C)O1]

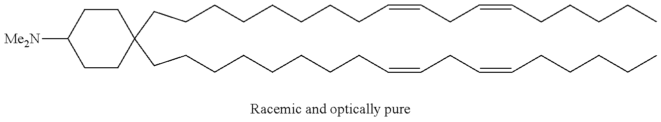 [SMILES: CCCCC/C=C\C/C=C\CCCCCCCCC1(CCCCCCCC/C=C\C/C=C\CCCCC)CCC(N(C)C)CC1]

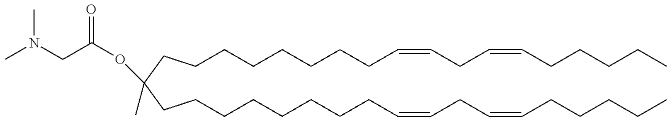 [SMILES: CCCCC/C=C\C/C=C\CCCCCCCCC(C)(CCCCCCCC/C=C\C/C=C\CCCCC)OC(=O)CN(C)C]

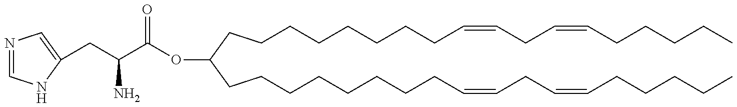 [SMILES: CCCCC/C=C\C/C=C\CCCCCCCCC(CCCCCCCC/C=C\C/C=C\CCCCC)OC(=O)[C@@H](N)Cc1cnc[nH]1]

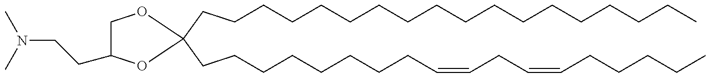 [SMILES: CCCCC/C=C\C/C=C\CCCCCCCCC1(CCCCCCCCCCCCCCCCCC)OCC(CCN(C)C)O1]

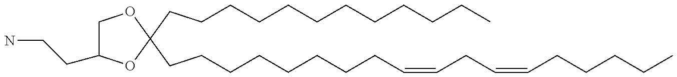 [SMILES: CCCCC/C=C\C/C=C\CCCCCCCCC1(CCCCCCCCCCCC)OCC(CCN)O1]

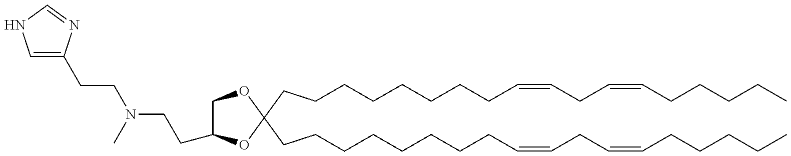

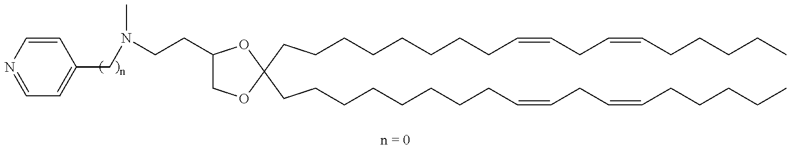

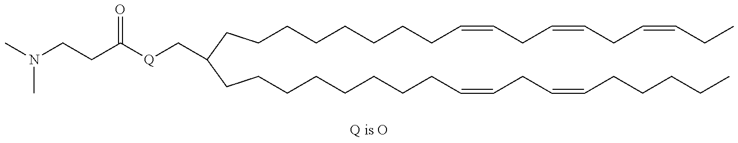

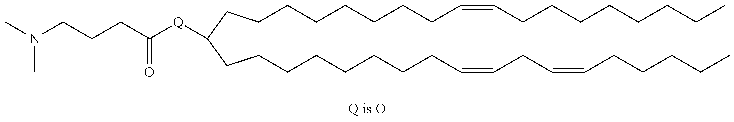

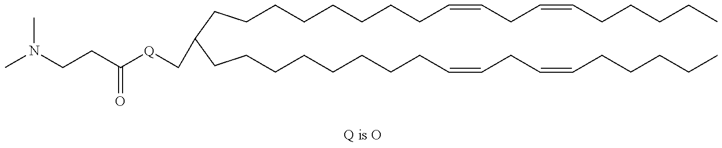

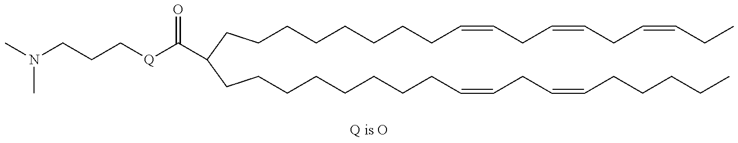

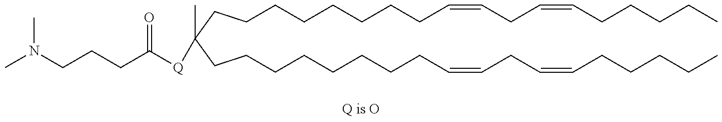

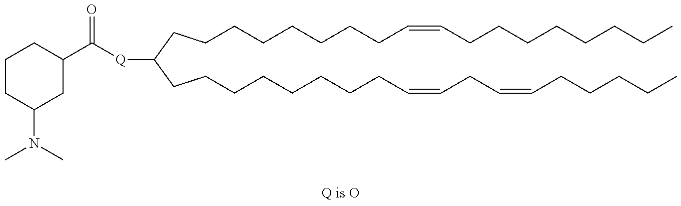

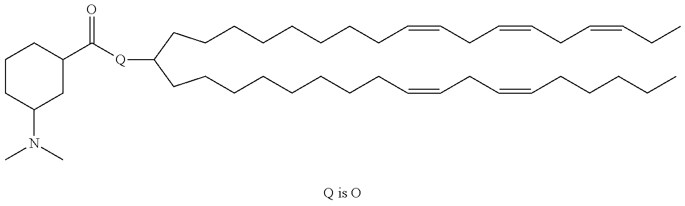

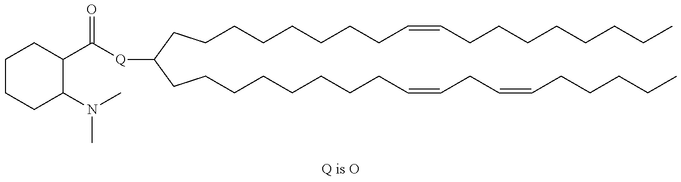

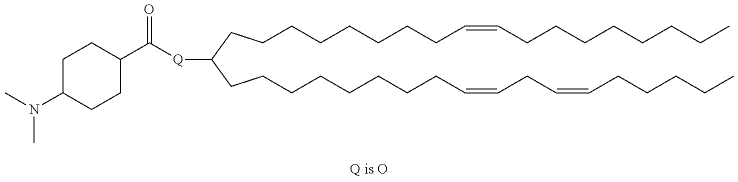

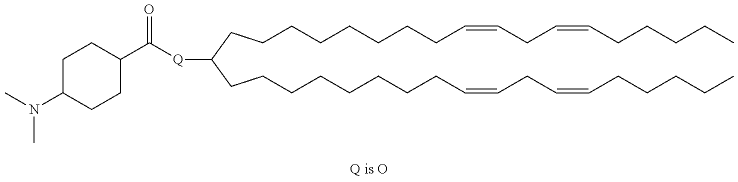

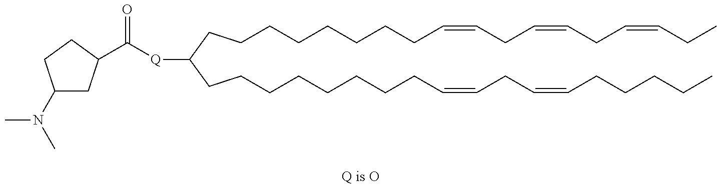

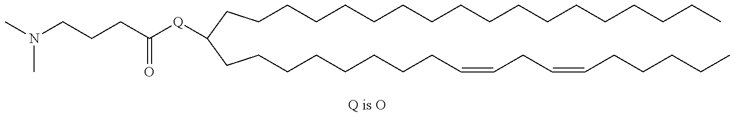

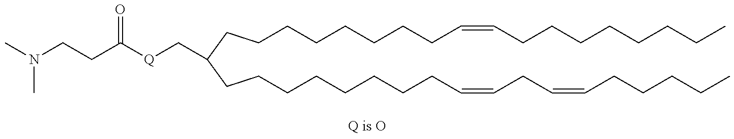

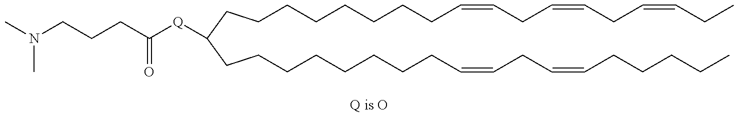

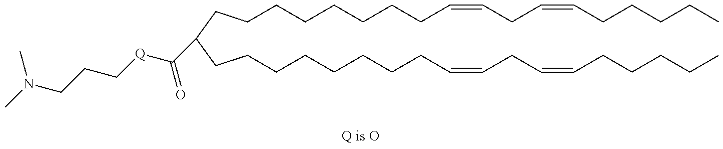

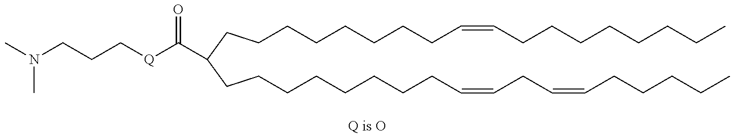

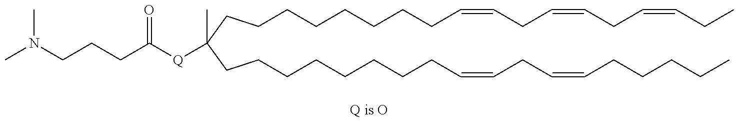

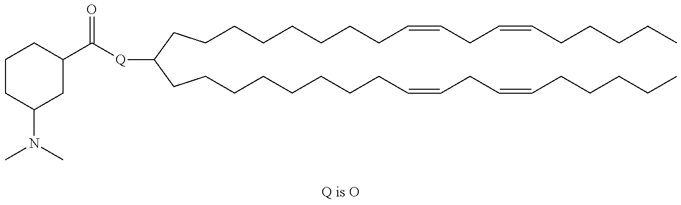

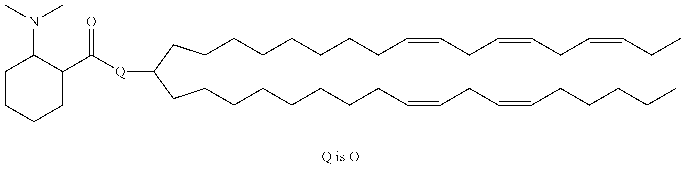

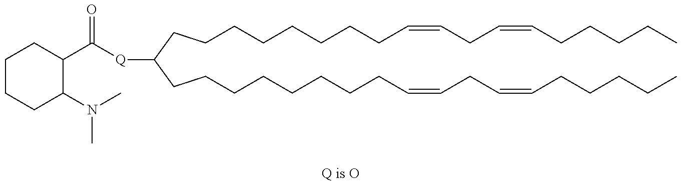

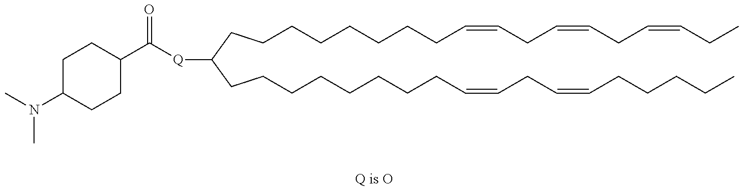

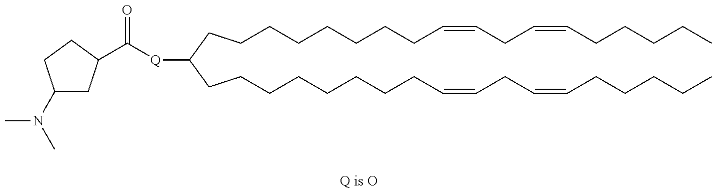

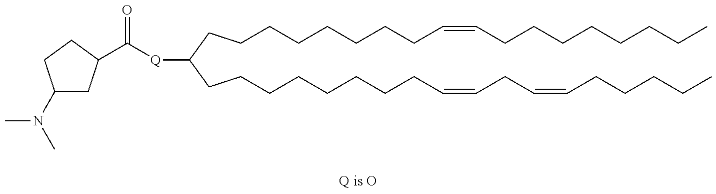

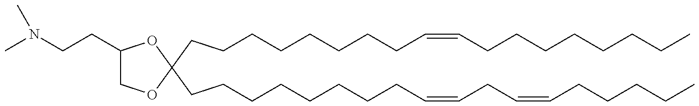 [SMILES: CCCCC/C=C\C/C=C\CCCCCCCCC1(CCCCCCCC/C=C\CCCCCCCC)OCC(CCN(C)C)O1]

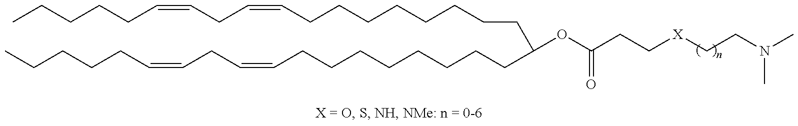

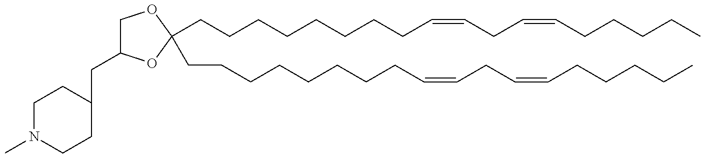 [SMILES: CCCCC/C=C\C/C=C\CCCCCCCCC1(CCCCCCCC/C=C\C/C=C\CCCCC)OCC(CC2CCN(C)CC2)O1]

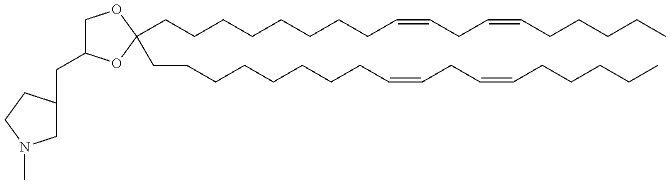 [SMILES: CCCCC/C=C\C/C=C\CCCCCCCCC1(CCCCCCCC/C=C\C/C=C\CCCCC)OCC(CC2CCN(C)C2)O1]

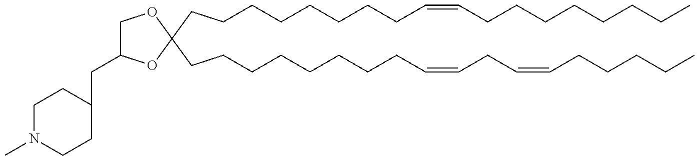 [SMILES: CCCCC/C=C\C/C=C\CCCCCCCCC1(CCCCCCCC/C=C\CCCCCCCC)OCC(CC2CCN(C)CC2)O1]

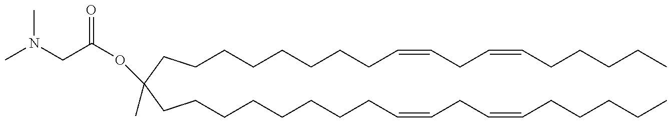 [SMILES: CCCCC/C=C\C/C=C\CCCCCCCCC(C)(CCCCCCCC/C=C\C/C=C\CCCCC)OC(=O)CN(C)C]

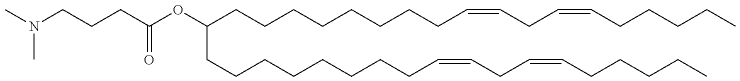 [SMILES: CCCCC/C=C\C/C=C\CCCCCCCCC(CCCCCCCC/C=C\C/C=C\CCCCC)OC(=O)CCCN(C)C]

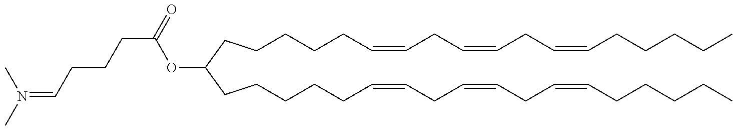

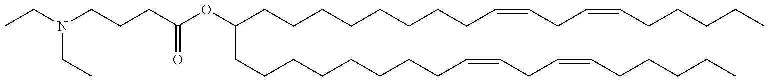 [SMILES: CCCCC/C=C\C/C=C\CCCCCCCCC(CCCCCCCC/C=C\C/C=C\CCCCC)OC(=O)CCCN(CC)CC]

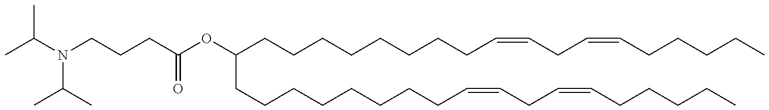 [SMILES: CCCCC/C=C\C/C=C\CCCCCCCCC(CCCCCCCC/C=C\C/C=C\CCCCC)OC(=O)CCCN(C(C)C)C(C)C]

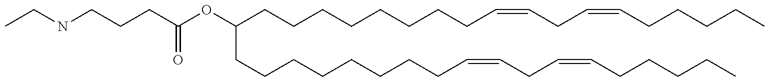 [SMILES: CCCCC/C=C\C/C=C\CCCCCCCCC(CCCCCCCC/C=C\C/C=C\CCCCC)OC(=O)CCCNCC]

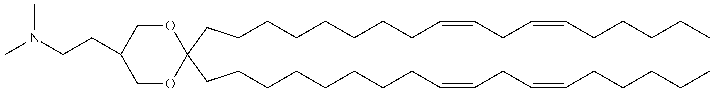 [SMILES: CCCCC/C=C\C/C=C\CCCCCCCCC1(CCCCCCCC/C=C\C/C=C\CCCCC)OCC(CCN(C)C)CO1]

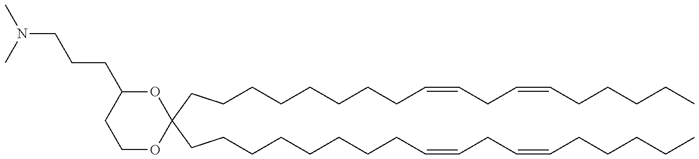 [SMILES: CCCCC/C=C\C/C=C\CCCCCCCCC1(CCCCCCCC/C=C\C/C=C\CCCCC)OCCC(CCCN(C)C)O1]

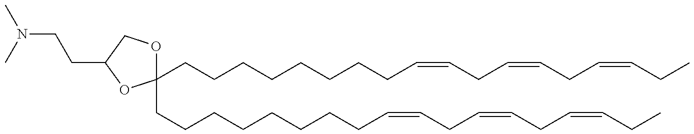 [SMILES: CC/C=C\C/C=C\C/C=C\CCCCCCCCC1(CCCCCCCC/C=C\C/C=C\C/C=C\CC)OCC(CCN(C)C)O1]

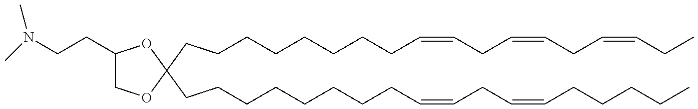 [SMILES: CC/C=C\C/C=C\C/C=C\CCCCCCCCC1(CCCCCCCC/C=C\C/C=C\CCCCC)OCC(CCN(C)C)O1]

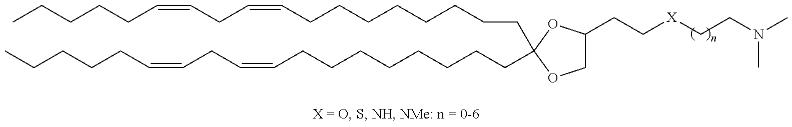

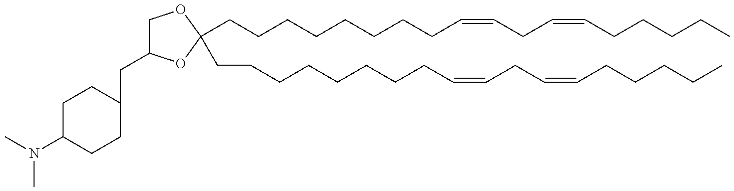 [SMILES: CCCCC/C=C\C/C=C\CCCCCCCCC1(CCCCCCCC/C=C\C/C=C\CCCCC)OCC(CC2CCC(N(C)C)CC2)O1]

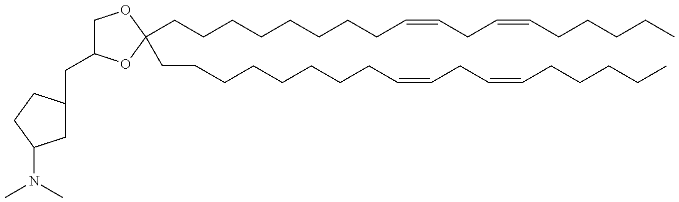 [SMILES: CCCCC/C=C\C/C=C\CCCCCCCCC1(CCCCCCCC/C=C\C/C=C\CCCCC)OCC(CC2CCC(N(C)C)C2)O1]

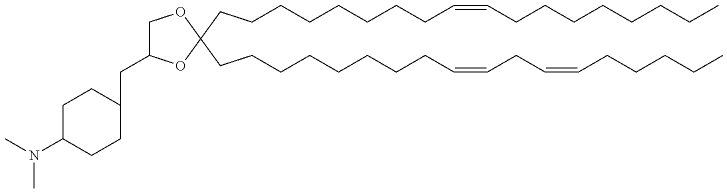 [SMILES: CCCCC/C=C\C/C=C\CCCCCCCCC1(CCCCCCCC/C=C\CCCCCCCC)OCC(CC2CCC(N(C)C)CC2)O1]

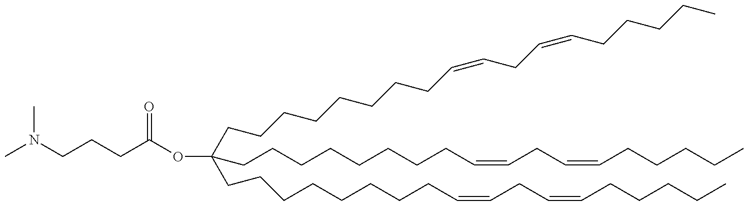 [SMILES: CCCCC/C=C\C/C=C\CCCCCCCCC(CCCCCCCC/C=C\C/C=C\CCCCC)(CCCCCCCC/C=C\C/C=C\CCCCC)OC(=O)CCCN(C)C]

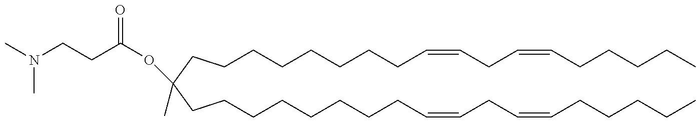 [SMILES: CCCCC/C=C\C/C=C\CCCCCCCCC(C)(CCCCCCCC/C=C\C/C=C\CCCCC)OC(=O)CCN(C)C]

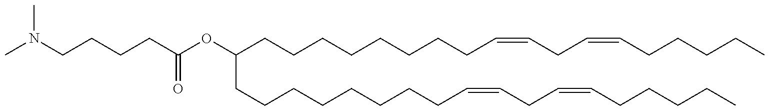 [SMILES: CCCCC/C=C\C/C=C\CCCCCCCCC(CCCCCCCC/C=C\C/C=C\CCCCC)OC(=O)CCCCN(C)C]

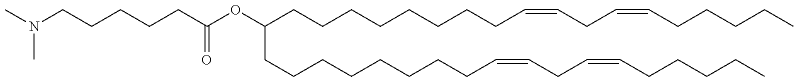 [SMILES: CCCCC/C=C\C/C=C\CCCCCCCCC(CCCCCCCC/C=C\C/C=C\CCCCC)OC(=O)CCCCCN(C)C]

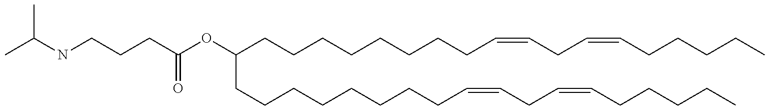 [SMILES: CCCCC/C=C\C/C=C\CCCCCCCCC(CCCCCCCC/C=C\C/C=C\CCCCC)OC(=O)CCCNC(C)C]

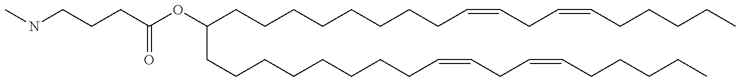 [SMILES: CCCCC/C=C\C/C=C\CCCCCCCCC(CCCCCCCC/C=C\C/C=C\CCCCC)OC(=O)CCCNC]

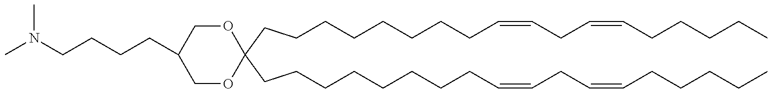 [SMILES: CCCCC/C=C\C/C=C\CCCCCCCCC1(CCCCCCCC/C=C\C/C=C\CCCCC)OCC(CCCCN(C)C)CO1]

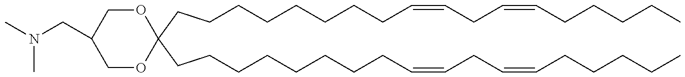 [SMILES: CCCCC/C=C\C/C=C\CCCCCCCCC1(CCCCCCCC/C=C\C/C=C\CCCCC)OCC(CN(C)C)CO1]

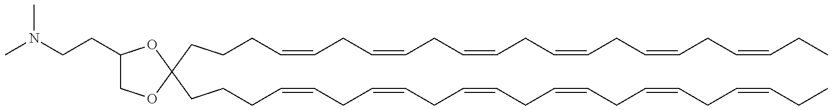 [SMILES: CC/C=C\C/C=C\C/C=C\C/C=C\C/C=C\C/C=C\CCCC1(CCC/C=C\C/C=C\C/C=C\C/C=C\C/C=C\C/C=C\CC)OCC(CCN(C)C)O1]

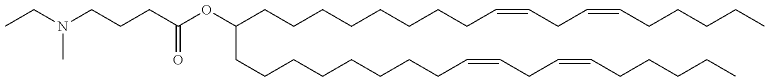 [SMILES: CCCCC/C=C\C/C=C\CCCCCCCCC(CCCCCCCC/C=C\C/C=C\CCCCC)OC(=O)CCCN(C)CC]

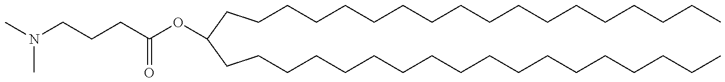 [SMILES: CCCCCCCCCCCCCCCCCCC(CCCCCCCCCCCCCCCCCC)OC(=O)CCCN(C)C]

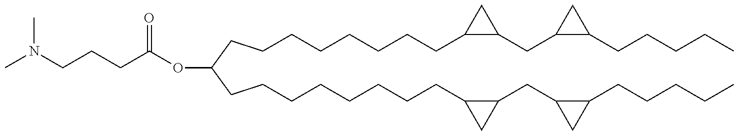 [SMILES: CCCCCC1CC1CC1CC1CCCCCCCCC(CCCCCCCCC1CC1CC1CC1CCCCC)OC(=O)CCCN(C)C]

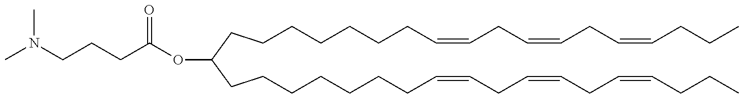 [SMILES: CCC/C=C\C/C=C\C/C=C\CCCCCCCC(CCCCCCC/C=C\C/C=C\C/C=C\CCC)OC(=O)CCCN(C)C]

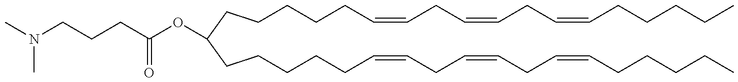 [SMILES: CCCCC/C=C\C/C=C\C/C=C\CCCCCC(CCCCC/C=C\C/C=C\C/C=C\CCCCC)OC(=O)CCCN(C)C]

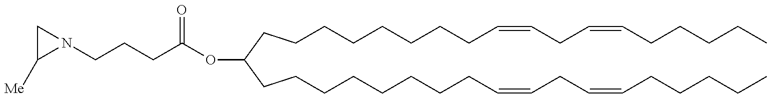 [SMILES: CCCCC/C=C\C/C=C\CCCCCCCCC(CCCCCCCC/C=C\C/C=C\CCCCC)OC(=O)CCCN1CC1C]

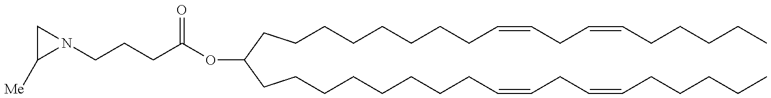 [SMILES: CCCCC/C=C\C/C=C\CCCCCCCCC(CCCCCCCC/C=C\C/C=C\CCCCC)OC(=O)CCCN1CC1C]

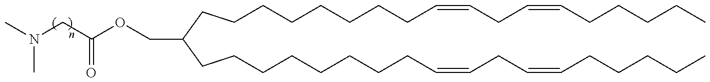

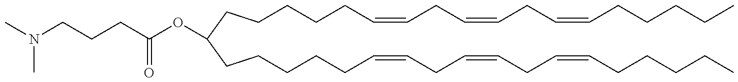 [SMILES: CCCCC/C=C\C/C=C\C/C=C\CCCCCC(CCCCC/C=C\C/C=C\C/C=C\CCCCC)OC(=O)CCCN(C)C]

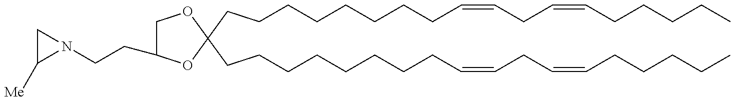 [SMILES: CCCCC/C=C\C/C=C\CCCCCCCCC1(CCCCCCCC/C=C\C/C=C\CCCCC)OCC(CCN2CC2C)O1]

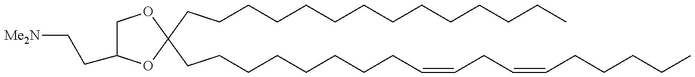 [SMILES: CCCCC/C=C\C/C=C\CCCCCCCCC1(CCCCCCCCCCCCCC)OCC(CCN(C)C)O1]

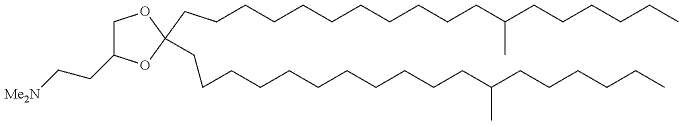 [SMILES: CCCCCCC(C)CCCCCCCCCCCC1(CCCCCCCCCCCC(C)CCCCCC)OCC(CCN(C)C)O1]

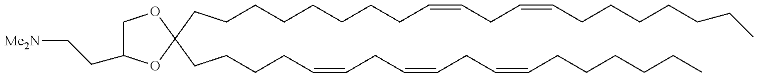 [SMILES: CCCCCC/C=C\C/C=C\C/C=C\CCCCC1(CCCCCCCC/C=C\C/C=C\CCCCCCC)OCC(CCN(C)C)O1]

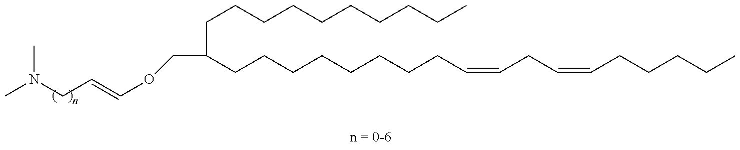

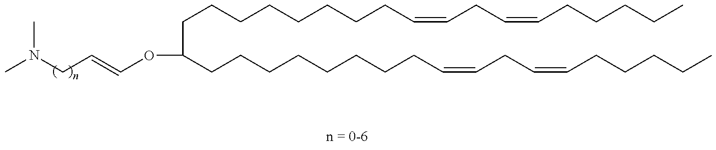

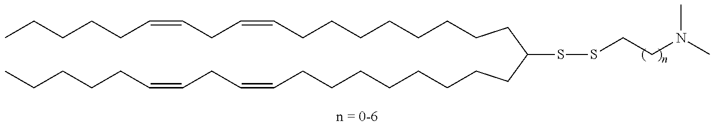

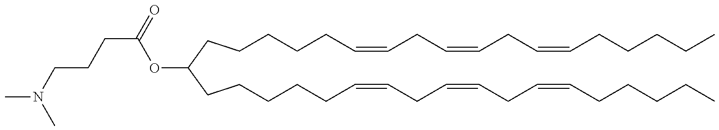 [SMILES: CCCCC/C=C\C/C=C\C/C=C\CCCCCC(CCCCC/C=C\C/C=C\C/C=C\CCCCC)OC(=O)CCCN(C)C]

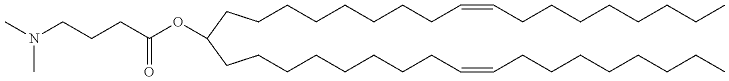 [SMILES: CCCCCCCC/C=C\CCCCCCCCC(CCCCCCCC/C=C\CCCCCCCC)OC(=O)CCCN(C)C]

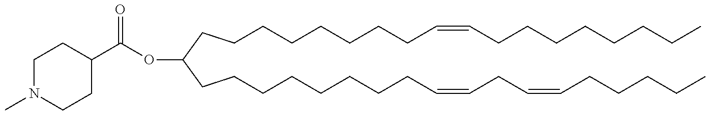 [SMILES: CCCCC/C=C\C/C=C\CCCCCCCCC(CCCCCCCC/C=C\CCCCCCCC)OC(=O)C1CCN(C)CC1]

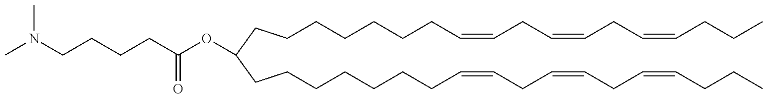 [SMILES: CCC/C=C\C/C=C\C/C=C\CCCCCCCC(CCCCCCC/C=C\C/C=C\C/C=C\CCC)OC(=O)CCCCN(C)C]

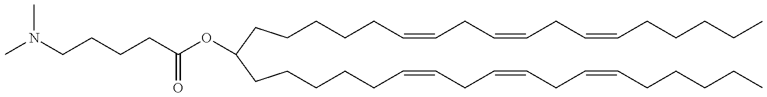 [SMILES: CCCCC/C=C\C/C=C\C/C=C\CCCCCC(CCCCC/C=C\C/C=C\C/C=C\CCCCC)OC(=O)CCCCN(C)C]

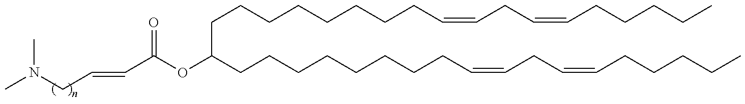

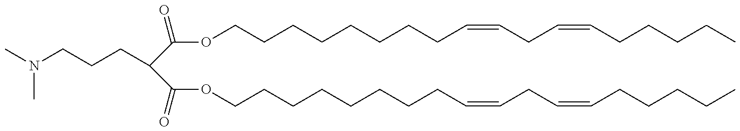 [SMILES: CCCCC/C=C\C/C=C\CCCCCCCCOC(=O)C(CCCN(C)C)C(=O)OCCCCCCCC/C=C\C/C=C\CCCCC]

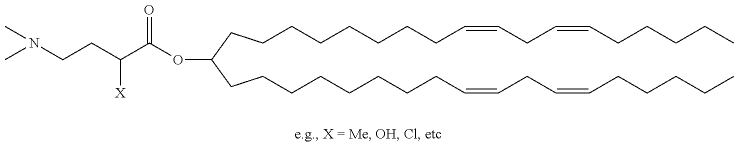

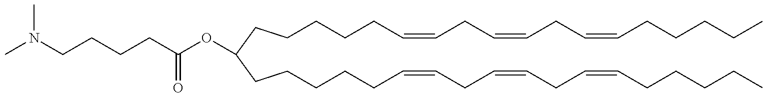 [SMILES: CCCCC/C=C\C/C=C\C/C=C\CCCCCC(CCCCC/C=C\C/C=C\C/C=C\CCCCC)OC(=O)CCCCN(C)C]

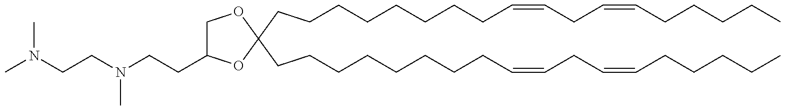 [SMILES: CCCCC/C=C\C/C=C\CCCCCCCCC1(CCCCCCCC/C=C\C/C=C\CCCCC)OCC(CCN(C)CCN(C)C)O1]

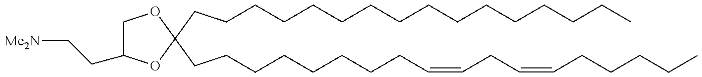 [SMILES: CCCCC/C=C\C/C=C\CCCCCCCCC1(CCCCCCCCCCCCCCCC)OCC(CCN(C)C)O1]

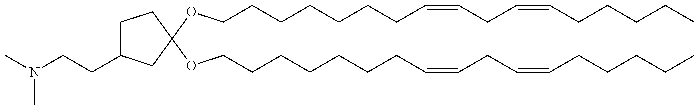 [SMILES: CCCCC/C=C\C/C=C\CCCCCCCOC1(OCCCCCCC/C=C\C/C=C\CCCCC)CCC(CCN(C)C)C1]

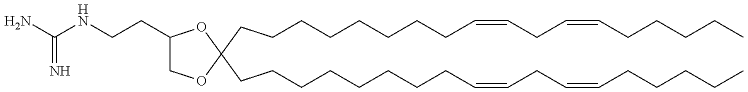 [SMILES: CCCCC/C=C\C/C=C\CCCCCCCCC1(CCCCCCCC/C=C\C/C=C\CCCCC)OCC(CCNC(=N)N)O1]

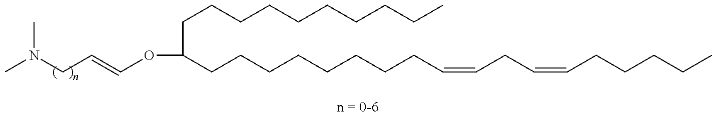

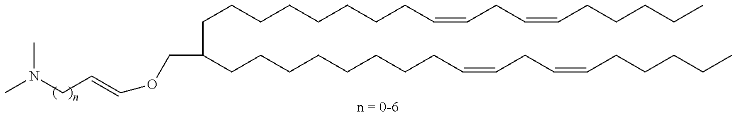

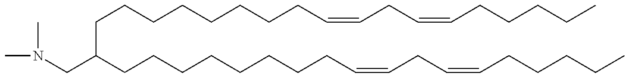 [SMILES: CCCCC/C=C\C/C=C\CCCCCCCCC(CCCCCCCC/C=C\C/C=C\CCCCC)CN(C)C]

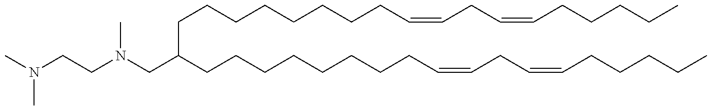 [SMILES: CCCCC/C=C\C/C=C\CCCCCCCCC(CCCCCCCC/C=C\C/C=C\CCCCC)CN(C)CCN(C)C]

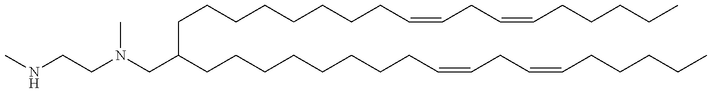 [SMILES: CCCCC/C=C\C/C=C\CCCCCCCCC(CCCCCCCC/C=C\C/C=C\CCCCC)CN(C)CCNC]

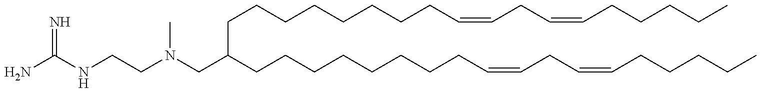 [SMILES: CCCCC/C=C\C/C=C\CCCCCCCCC(CCCCCCCC/C=C\C/C=C\CCCCC)CN(C)CCNC(=N)N]

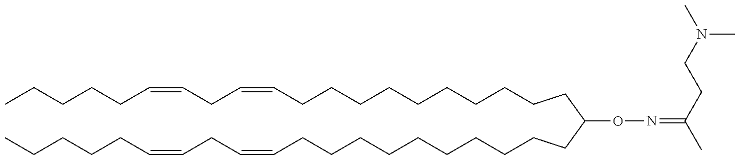 [SMILES: CCCCC/C=C\C/C=C\CCCCCCCCCCC(CCCCCCCCCC/C=C\C/C=C\CCCCC)ON=C(C)CCN(C)C]

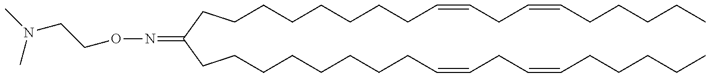 [SMILES: CCCCC/C=C\C/C=C\CCCCCCCCC(CCCCCCCC/C=C\C/C=C\CCCCC)=NOCCN(C)C]

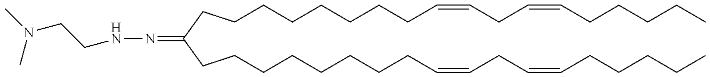 [SMILES: CCCCC/C=C\C/C=C\CCCCCCCCC(CCCCCCCC/C=C\C/C=C\CCCCC)=NNCCN(C)C]

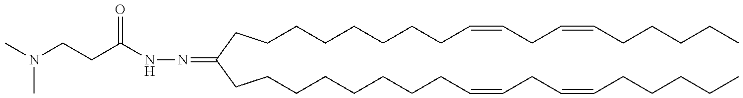 [SMILES: CCCCC/C=C\C/C=C\CCCCCCCCC(CCCCCCCC/C=C\C/C=C\CCCCC)=NNC(=O)CCN(C)C]

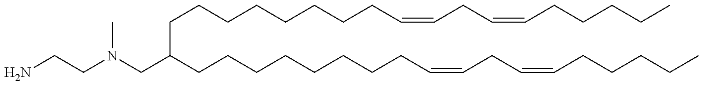 [SMILES: CCCCC/C=C\C/C=C\CCCCCCCCC(CCCCCCCC/C=C\C/C=C\CCCCC)CN(C)CCN]

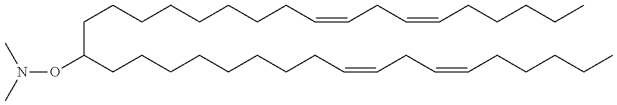 [SMILES: CCCCC/C=C\C/C=C\CCCCCCCCC(CCCCCCCC/C=C\C/C=C\CCCCC)ON(C)C]

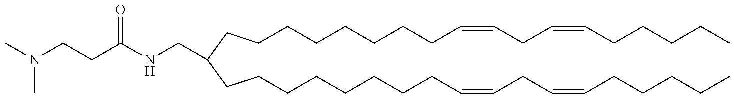 [SMILES: CCCCC/C=C\C/C=C\CCCCCCCCC(CCCCCCCC/C=C\C/C=C\CCCCC)CNC(=O)CCN(C)C]

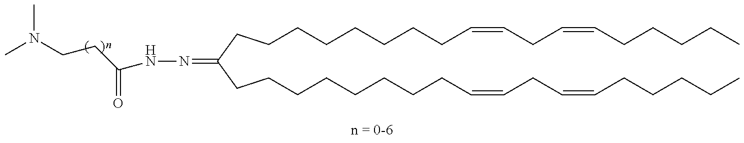

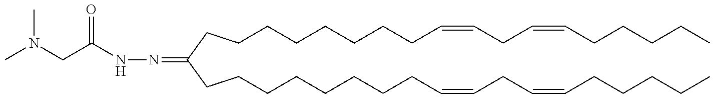 [SMILES: CCCCC/C=C\C/C=C\CCCCCCCCC(CCCCCCCC/C=C\C/C=C\CCCCC)=NNC(=O)CN(C)C]

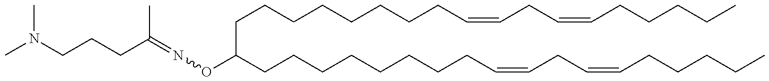 [SMILES: CCCCC/C=C\C/C=C\CCCCCCCCC(CCCCCCCC/C=C\C/C=C\CCCCC)ON=C(C)CCCN(C)C]

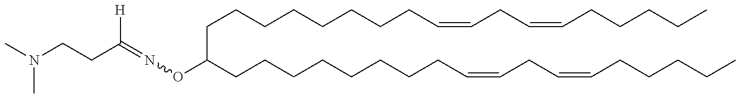 [SMILES: CCCCC/C=C\C/C=C\CCCCCCCCC(CCCCCCCC/C=C\C/C=C\CCCCC)ON=CCCN(C)C]

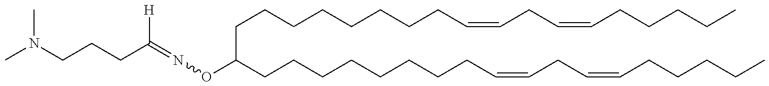 [SMILES: CCCCC/C=C\C/C=C\CCCCCCCCC(CCCCCCCC/C=C\C/C=C\CCCCC)ON=CCCCN(C)C]

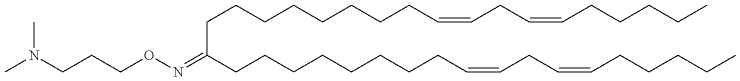 [SMILES: CCCCC/C=C\C/C=C\CCCCCCCCC(CCCCCCCC/C=C\C/C=C\CCCCC)=NOCCCN(C)C]

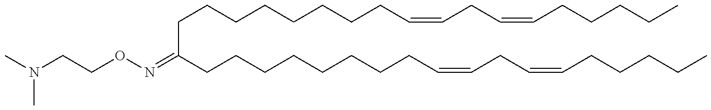 [SMILES: CCCCC/C=C\C/C=C\CCCCCCCCC(CCCCCCCC/C=C\C/C=C\CCCCC)=NOCCN(C)C]

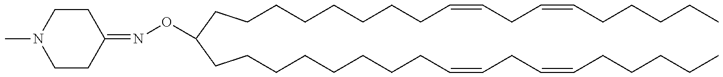 [SMILES: CCCCC/C=C\C/C=C\CCCCCCCCC(CCCCCCCC/C=C\C/C=C\CCCCC)ON=C1CCN(C)CC1]

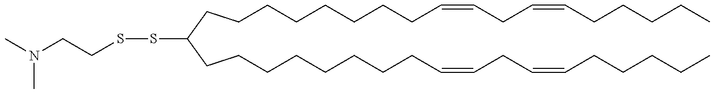 [SMILES: CCCCC/C=C\C/C=C\CCCCCCCCC(CCCCCCCC/C=C\C/C=C\CCCCC)SSCCN(C)C]

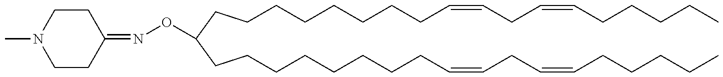 [SMILES: CCCCC/C=C\C/C=C\CCCCCCCCC(CCCCCCCC/C=C\C/C=C\CCCCC)ON=C1CCN(C)CC1]

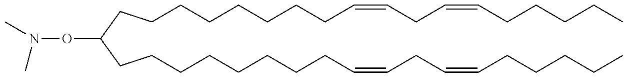 [SMILES: CCCCC/C=C\C/C=C\CCCCCCCCC(CCCCCCCC/C=C\C/C=C\CCCCC)ON(C)C]

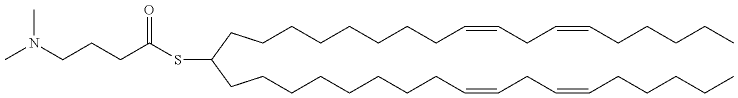 [SMILES: CCCCC/C=C\C/C=C\CCCCCCCCC(CCCCCCCC/C=C\C/C=C\CCCCC)SC(=O)CCCN(C)C]

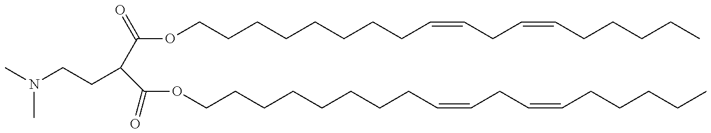 [SMILES: CCCCC/C=C\C/C=C\CCCCCCCCOC(=O)C(CCN(C)C)C(=O)OCCCCCCCC/C=C\C/C=C\CCCCC]

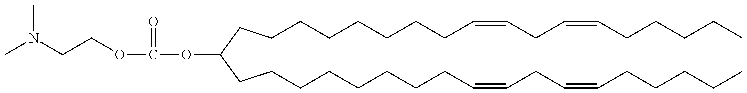 [SMILES: CCCCC/C=C\C/C=C\CCCCCCCCC(CCCCCCCC/C=C\C/C=C\CCCCC)OC(=O)OCCN(C)C]

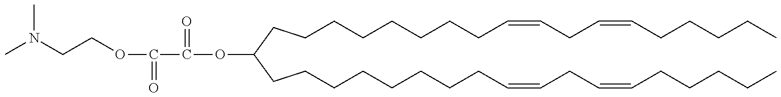 [SMILES: CCCCC/C=C\C/C=C\CCCCCCCCC(CCCCCCCC/C=C\C/C=C\CCCCC)OC(=O)C(=O)OCCN(C)C]

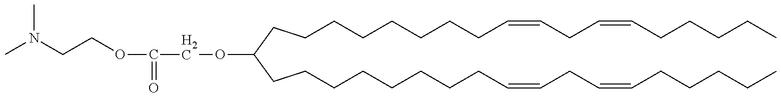 [SMILES: CCCCC/C=C\C/C=C\CCCCCCCCC(CCCCCCCC/C=C\C/C=C\CCCCC)OCC(=O)OCCN(C)C]

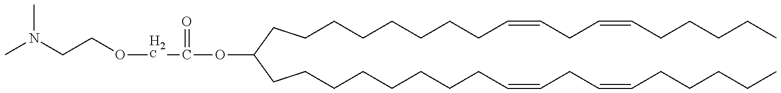 [SMILES: CCCCC/C=C\C/C=C\CCCCCCCCC(CCCCCCCC/C=C\C/C=C\CCCCC)OC(=O)COCCN(C)C]

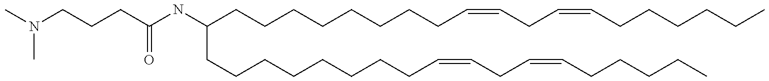 [SMILES: CCCCC/C=C\C/C=C\CCCCCCCCC(CCCCCCCC/C=C\C/C=C\CCCCCC)NC(=O)CCCN(C)C]

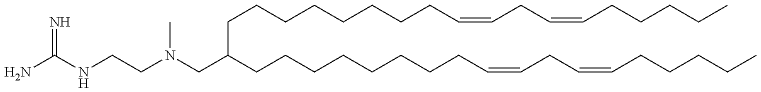 [SMILES: CCCCC/C=C\C/C=C\CCCCCCCCC(CCCCCCCC/C=C\C/C=C\CCCCC)CN(C)CCNC(=N)N]

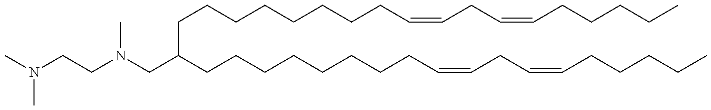 [SMILES: CCCCC/C=C\C/C=C\CCCCCCCCC(CCCCCCCC/C=C\C/C=C\CCCCC)CN(C)CCN(C)C]

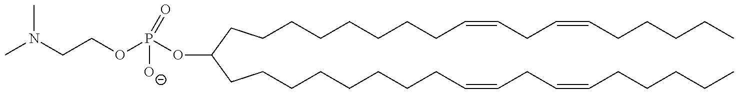 [SMILES: CCCCC/C=C\C/C=C\CCCCCCCCC(CCCCCCCC/C=C\C/C=C\CCCCC)OP(=O)([O-])OCCN(C)C]

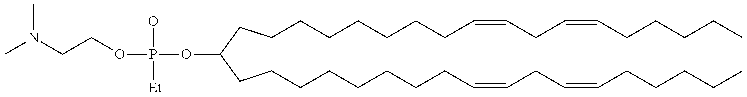

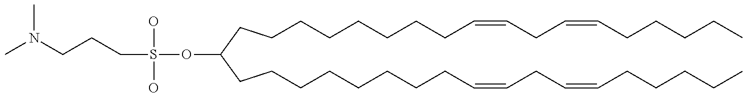 [SMILES: CCCCC/C=C\C/C=C\CCCCCCCCC(CCCCCCCC/C=C\C/C=C\CCCCC)OS(O)(O)CCCN(C)C]

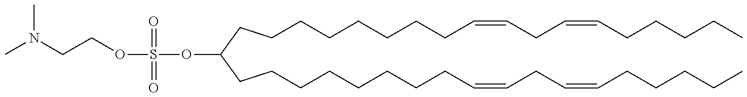 [SMILES: CCCCC/C=C\C/C=C\CCCCCCCCC(CCCCCCCC/C=C\C/C=C\CCCCC)OS(=O)(=O)OCCN(C)C]

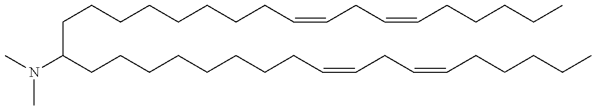 [SMILES: CCCCC/C=C\C/C=C\CCCCCCCCC(CCCCCCCC/C=C\C/C=C\CCCCC)N(C)C]

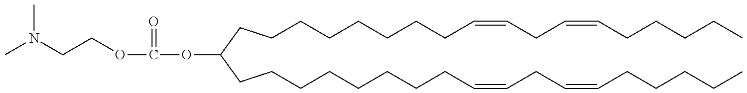 [SMILES: CCCCC/C=C\C/C=C\CCCCCCCCC(CCCCCCCC/C=C\C/C=C\CCCCC)OC(=O)OCCN(C)C]

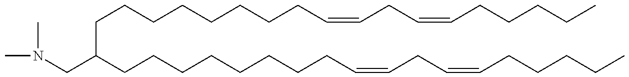 [SMILES: CCCCC/C=C\C/C=C\CCCCCCCCC(CCCCCCCC/C=C\C/C=C\CCCCC)CN(C)C]

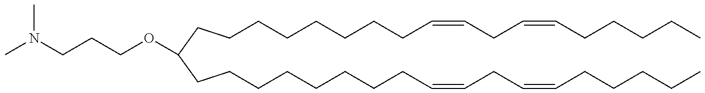 [SMILES: CCCCC/C=C\C/C=C\CCCCCCCCC(CCCCCCCC/C=C\C/C=C\CCCCC)OCCCN(C)C]

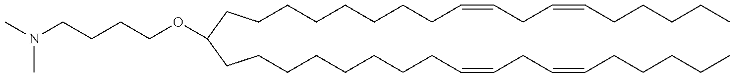 [SMILES: CCCCC/C=C\C/C=C\CCCCCCCCC(CCCCCCCC/C=C\C/C=C\CCCCC)OCCCCN(C)C]

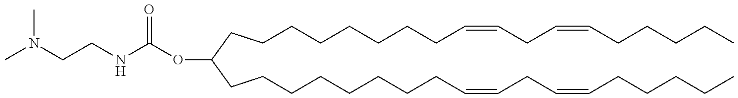 [SMILES: CCCCC/C=C\C/C=C\CCCCCCCCC(CCCCCCCC/C=C\C/C=C\CCCCC)OC(=O)NCCN(C)C]

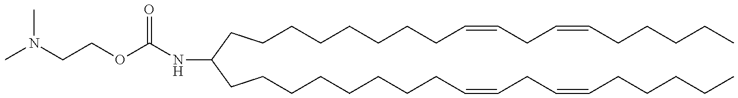 [SMILES: CCCCC/C=C\C/C=C\CCCCCCCCC(CCCCCCCC/C=C\C/C=C\CCCCC)NC(=O)OCCN(C)C]

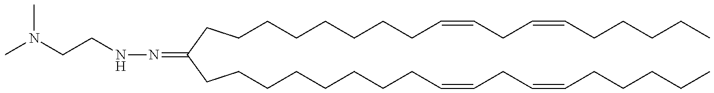 [SMILES: CCCCC/C=C\C/C=C\CCCCCCCCC(CCCCCCCC/C=C\C/C=C\CCCCC)=NNCCN(C)C]

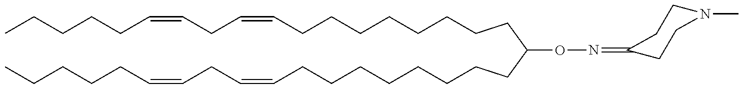 [SMILES: CCCCC/C=C\C/C=C\CCCCCCCCC(CCCCCCCC/C=C\C/C=C\CCCCC)ON=C1CCN(C)CC1]

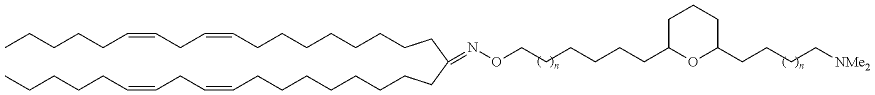

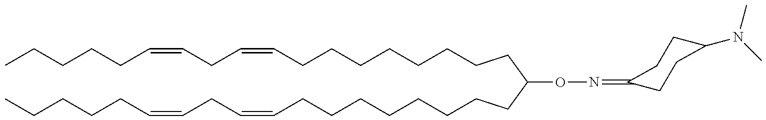 [SMILES: CCCCC/C=C\C/C=C\CCCCCCCCC(CCCCCCCC/C=C\C/C=C\CCCCC)ON=C1CCC(N(C)C)CC1]

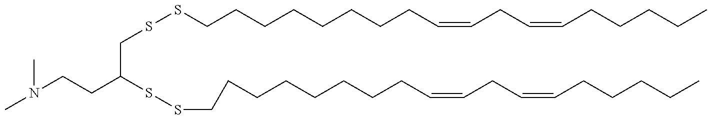 [SMILES: CCCCC/C=C\C/C=C\CCCCCCCCSSCC(CCN(C)C)SSCCCCCCCC/C=C\C/C=C\CCCCC]

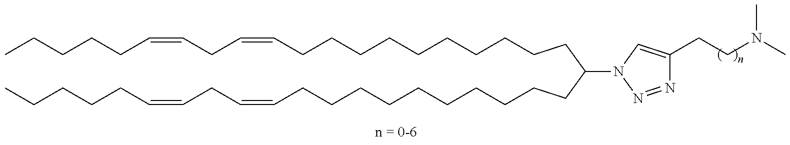

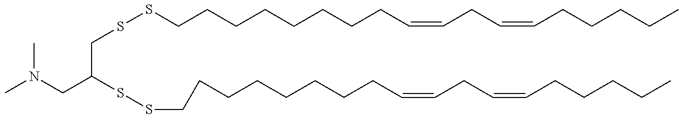 [SMILES: CCCCC/C=C\C/C=C\CCCCCCCCSSCC(CN(C)C)SSCCCCCCCC/C=C\C/C=C\CCCCC]

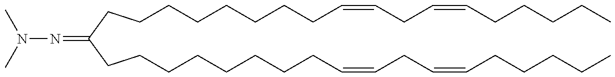 [SMILES: CCCCC/C=C\C/C=C\CCCCCCCCC(CCCCCCCC/C=C\C/C=C\CCCCC)=NN(C)C]

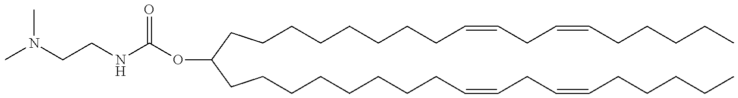 [SMILES: CCCCC/C=C\C/C=C\CCCCCCCCC(CCCCCCCC/C=C\C/C=C\CCCCC)OC(=O)NCCN(C)C]

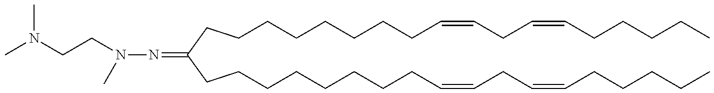 [SMILES: CCCCC/C=C\C/C=C\CCCCCCCCC(CCCCCCCC/C=C\C/C=C\CCCCC)=NN(C)CCN(C)C]

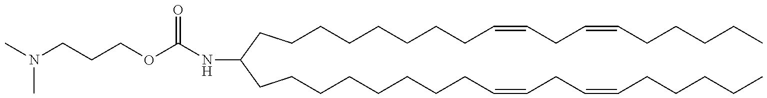 [SMILES: CCCCC/C=C\C/C=C\CCCCCCCCC(CCCCCCCC/C=C\C/C=C\CCCCC)NC(=O)OCCCN(C)C]

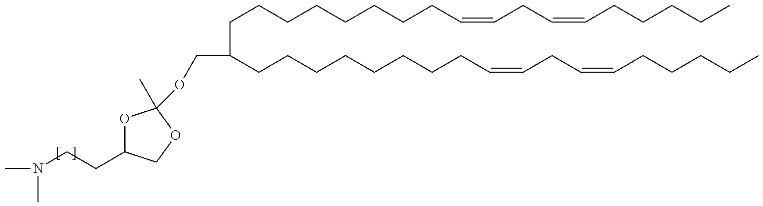

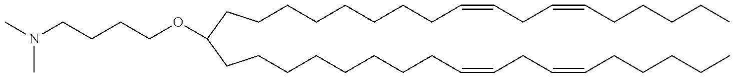 [SMILES: CCCCC/C=C\C/C=C\CCCCCCCCC(CCCCCCCC/C=C\C/C=C\CCCCC)OCCCCN(C)C]

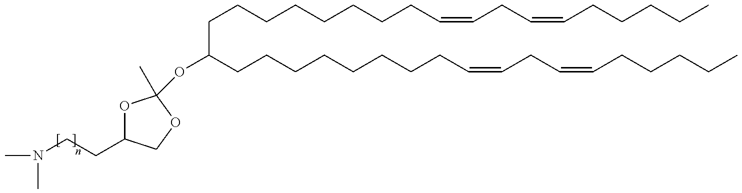

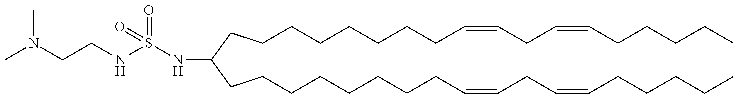 [SMILES: CCCCC/C=C\C/C=C\CCCCCCCCC(CCCCCCCC/C=C\C/C=C\CCCCC)NS(=O)(=O)NCCN(C)C]

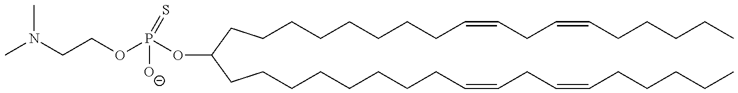 [SMILES: CCCCC/C=C\C/C=C\CCCCCCCCC(CCCCCCCC/C=C\C/C=C\CCCCC)OP([O-])(=S)OCCN(C)C]

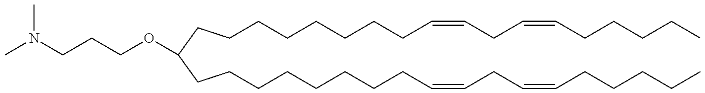 [SMILES: CCCCC/C=C\C/C=C\CCCCCCCCC(CCCCCCCC/C=C\C/C=C\CCCCC)OCCCN(C)C]

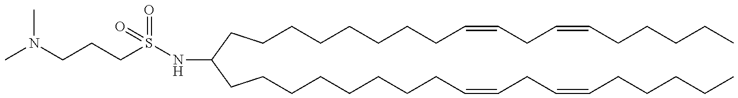 [SMILES: CCCCC/C=C\C/C=C\CCCCCCCCC(CCCCCCCC/C=C\C/C=C\CCCCC)NS(=O)(=O)CCCN(C)C]

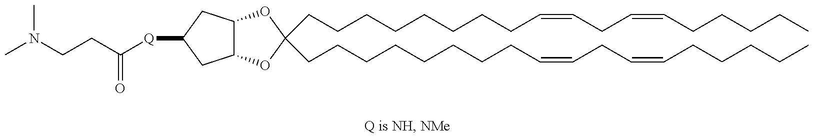

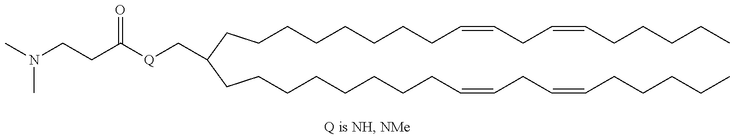

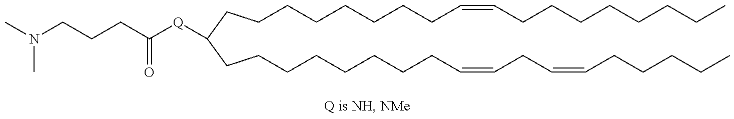

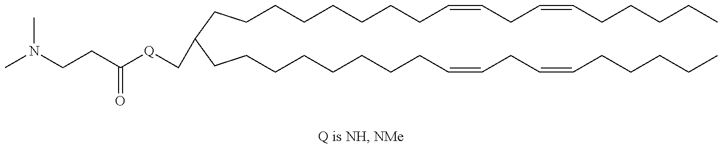

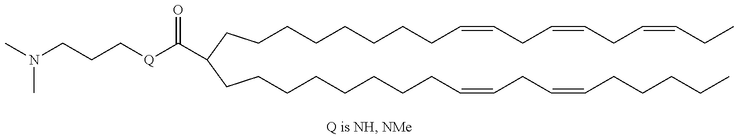

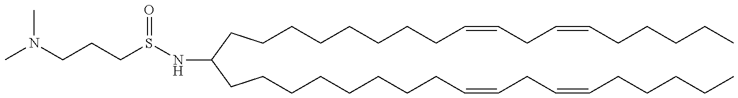 [SMILES: CCCCC/C=C\C/C=C\CCCCCCCCC(CCCCCCCC/C=C\C/C=C\CCCCC)NS(=O)CCCN(C)C]

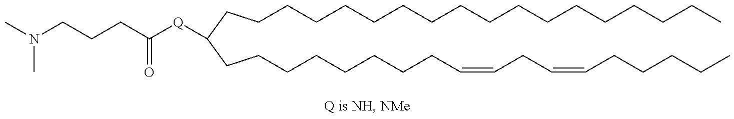

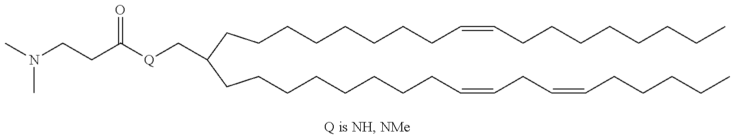

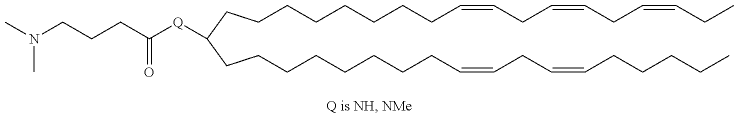

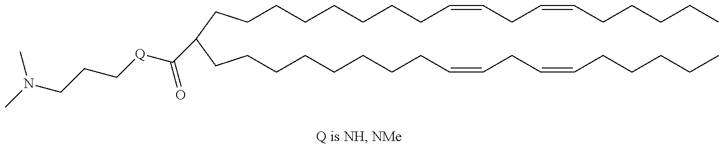

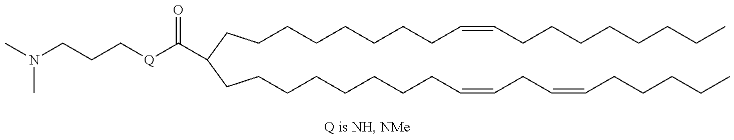

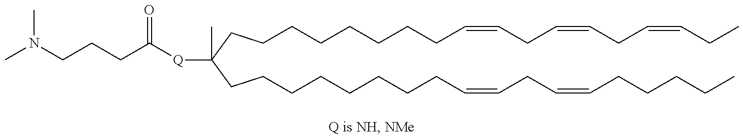

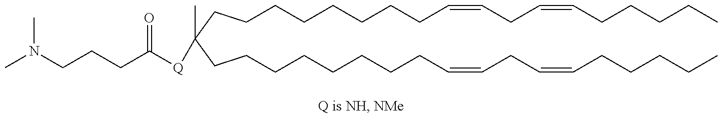

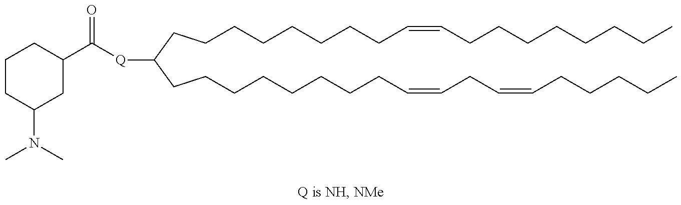

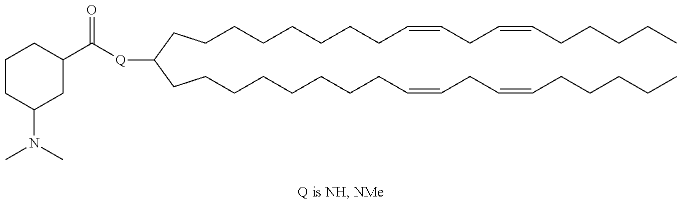

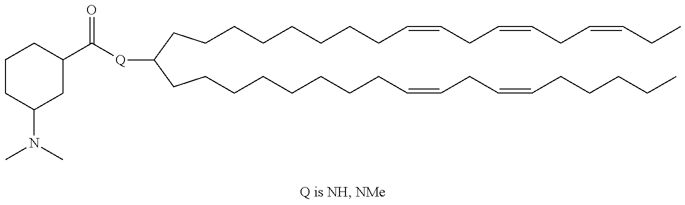

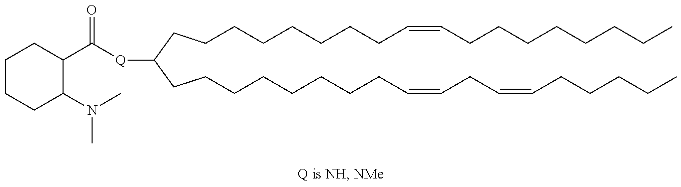

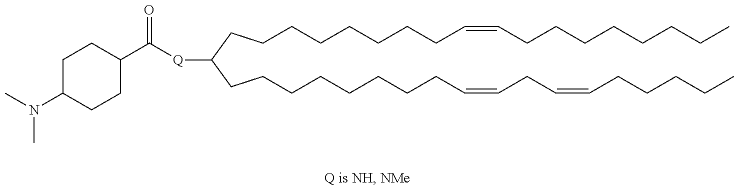

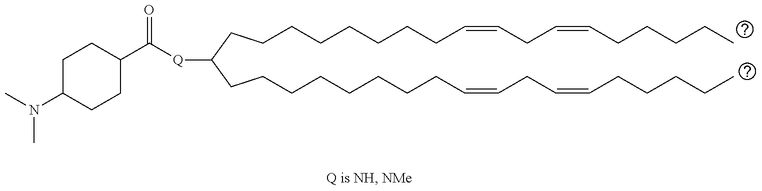

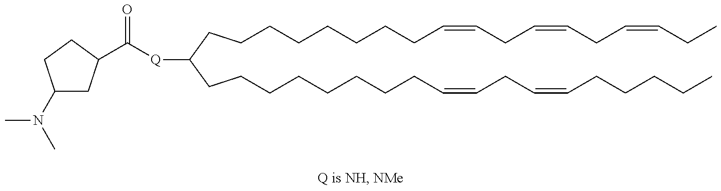

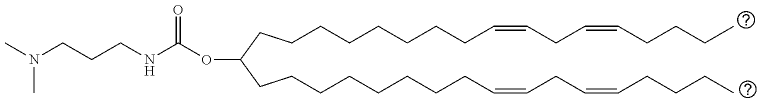 [SMILES: CCCC/C=C\C/C=C\CCCCCCCCC(CCCCCCCC/C=C\C/C=C\CCCC)OC(=O)NCCCN(C)C]

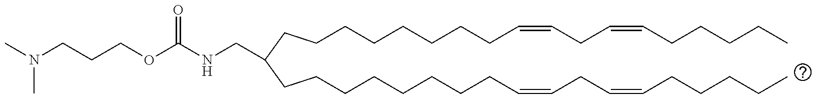 [SMILES: CCCCC/C=C\C/C=C\CCCCCCCCC(CCCCCCCC/C=C\C/C=C\CCCCC)CNC(=O)OCCCN(C)C]

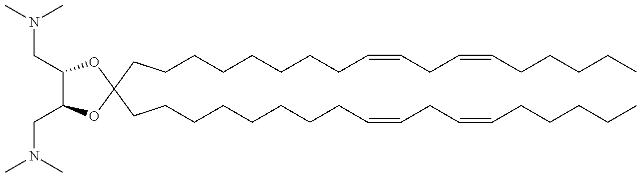 [SMILES: CCCCC/C=C\C/C=C\CCCCCCCCC1(CCCCCCCC/C=C\C/C=C\CCCCC)O[C@@H](CN(C)C)[C@H](CN(C)C)O1]

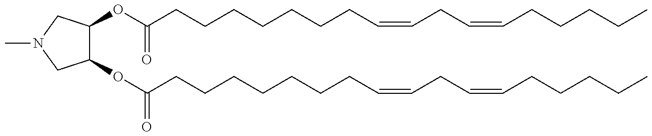 [SMILES: CCCCC/C=C\C/C=C\CCCCCCCC(=O)O[C@H]1CN(C)C[C@H]1OC(=O)CCCCCCC/C=C\C/C=C\CCCCC]

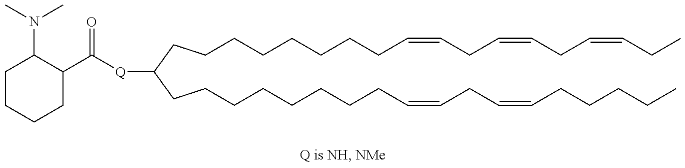

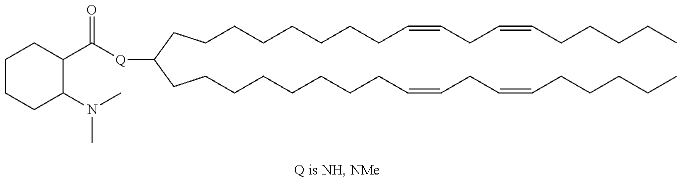

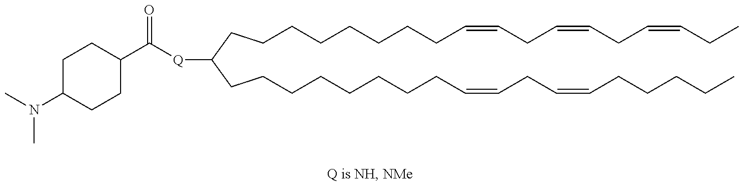

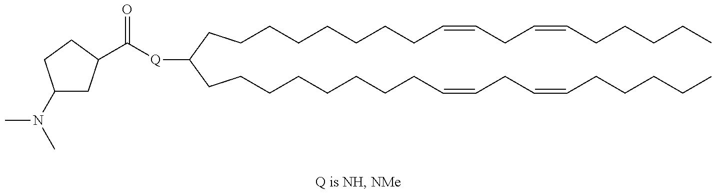

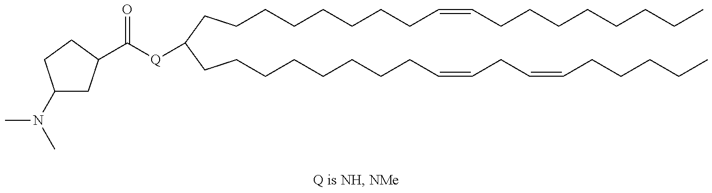

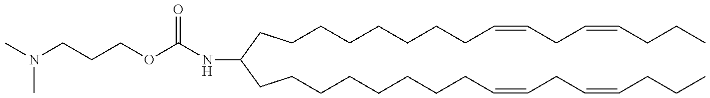 [SMILES: CCC/C=C\C/C=C\CCCCCCCCC(CCCCCCCC/C=C\C/C=C\CCC)NC(=O)OCCCN(C)C]

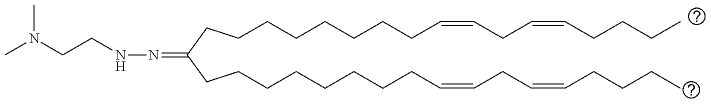 [SMILES: CCCC/C=C\C/C=C\CCCCCCCCC(CCCCCCCC/C=C\C/C=C\CCCC)=NNCCN(C)C]

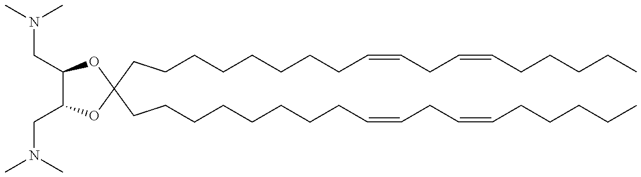 [SMILES: CCCCC/C=C\C/C=C\CCCCCCCCC1(CCCCCCCC/C=C\C/C=C\CCCCC)O[C@H](CN(C)C)[C@@H](CN(C)C)O1]

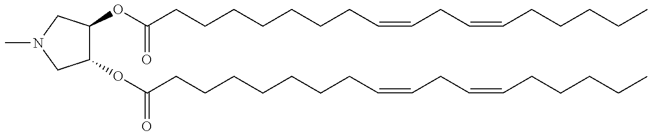 [SMILES: CCCCC/C=C\C/C=C\CCCCCCCC(=O)O[C@@H]1CN(C)C[C@H]1OC(=O)CCCCCCC/C=C\C/C=C\CCCCC]

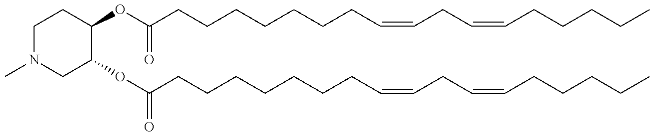 [SMILES: CCCCC/C=C\C/C=C\CCCCCCCC(=O)O[C@@H]1CCN(C)C[C@H]1OC(=O)CCCCCCC/C=C\C/C=C\CCCCC]

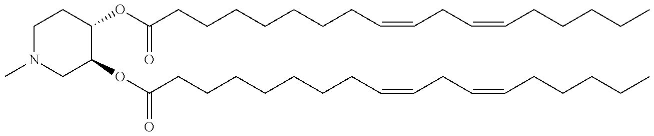 [SMILES: CCCCC/C=C\C/C=C\CCCCCCCC(=O)O[C@H]1CCN(C)C[C@@H]1OC(=O)CCCCCCC/C=C\C/C=C\CCCCC]

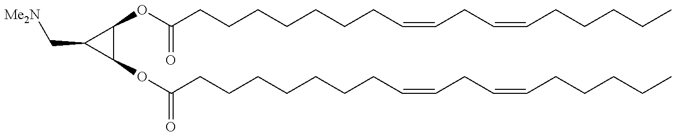 [SMILES: CCCCC/C=C\C/C=C\CCCCCCCC(=O)O[C@@H]1[C@H](CN(C)C)[C@@H]1OC(=O)CCCCCCC/C=C\C/C=C\CCCCC]

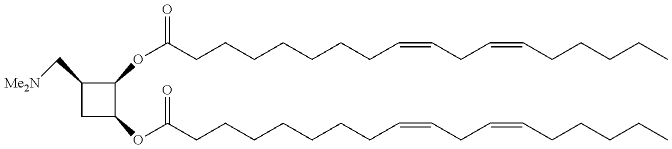 [SMILES: CCCCC/C=C\C/C=C\CCCCCCCC(=O)O[C@@H]1[C@H](CN(C)C)C[C@@H]1OC(=O)CCCCCCC/C=C\C/C=C\CCCCC]

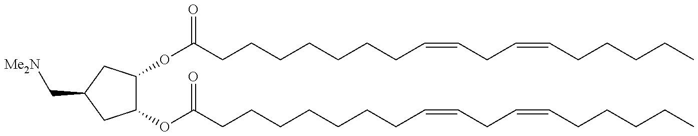 [SMILES: CCCCC/C=C\C/C=C\CCCCCCCC(=O)O[C@H]1C[C@H](CN(C)C)C[C@H]1OC(=O)CCCCCCC/C=C\C/C=C\CCCCC]

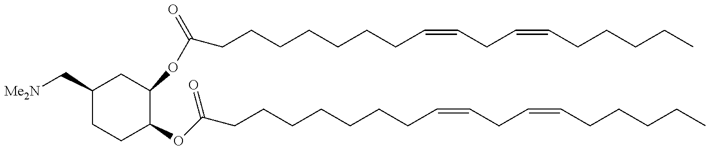 [SMILES: CCCCC/C=C\C/C=C\CCCCCCCC(=O)O[C@H]1CC[C@@H](CN(C)C)C[C@H]1OC(=O)CCCCCCC/C=C\C/C=C\CCCCC]

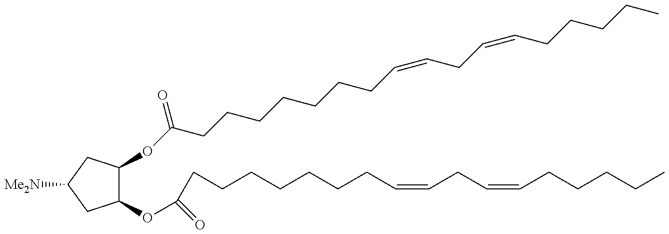 [SMILES: CCCCC/C=C\C/C=C\CCCCCCCC(=O)O[C@H]1C[C@H](N(C)C)C[C@H]1OC(=O)CCCCCCC/C=C\C/C=C\CCCCC]

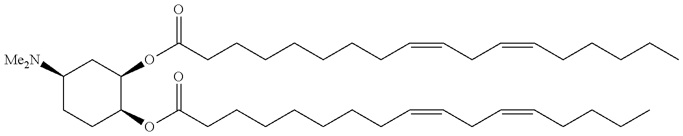 [SMILES: CCCC/C=C\C/C=C\CCCCCCCC(=O)O[C@H]1CC[C@@H](N(C)C)C[C@H]1OC(=O)CCCCCCC/C=C\C/C=C\CCCCC]

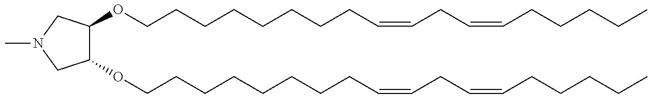 [SMILES: CCCCC/C=C\C/C=C\CCCCCCCCO[C@@H]1CN(C)C[C@H]1OCCCCCCCC/C=C\C/C=C\CCCCC]

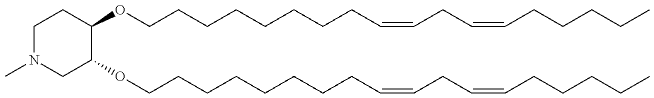 [SMILES: CCCCC/C=C\C/C=C\CCCCCCCCO[C@@H]1CCN(C)C[C@H]1OCCCCCCCC/C=C\C/C=C\CCCCC]

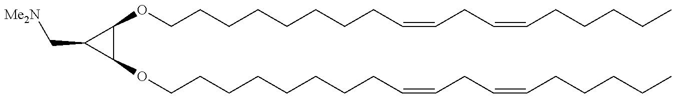 [SMILES: CCCCC/C=C\C/C=C\CCCCCCCCO[C@@H]1[C@H](CN(C)C)[C@@H]1OCCCCCCCC/C=C\C/C=C\CCCCC]

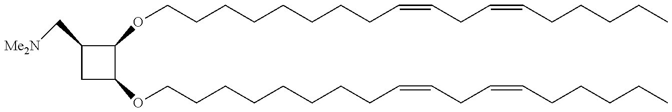 [SMILES: CCCCC/C=C\C/C=C\CCCCCCCCO[C@@H]1[C@H](CN(C)C)C[C@@H]1OCCCCCCCC/C=C\C/C=C\CCCCC]

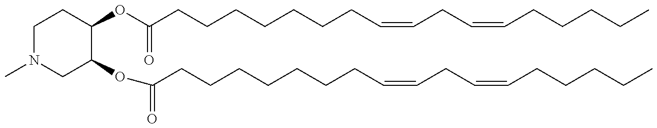 [SMILES: CCCCC/C=C\C/C=C\CCCCCCCC(=O)O[C@H]1CN(C)CC[C@H]1OC(=O)CCCCCCC/C=C\C/C=C\CCCCC]

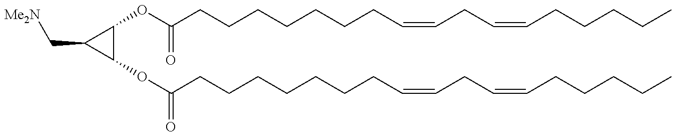 [SMILES: CCCCC/C=C\C/C=C\CCCCCCCC(=O)O[C@@H]1[C@@H](CN(C)C)[C@@H]1OC(=O)CCCCCCC/C=C\C/C=C\CCCCC]

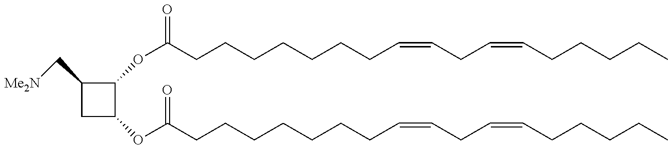 [SMILES: CCCCC/C=C\C/C=C\CCCCCCCC(=O)O[C@H]1[C@H](CN(C)C)C[C@H]1OC(=O)CCCCCCC/C=C\C/C=C\CCCCC]

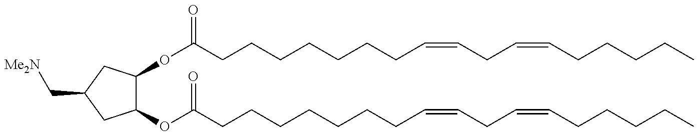 [SMILES: CCCCC/C=C\C/C=C\CCCCCCCC(=O)O[C@H]1C[C@@H](CN(C)C)C[C@H]1OC(=O)CCCCCCC/C=C\C/C=C\CCCCC]

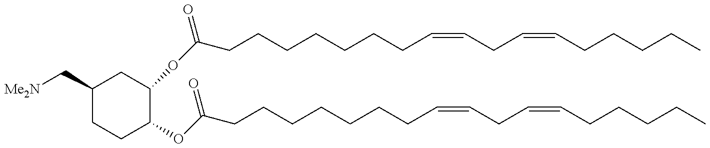 [SMILES: CCCCC/C=C\C/C=C\CCCCCCCC(=O)O[C@H]1C[C@H](CN(C)C)CC[C@H]1OC(=O)CCCCCCC/C=C\C/C=C\CCCCC]

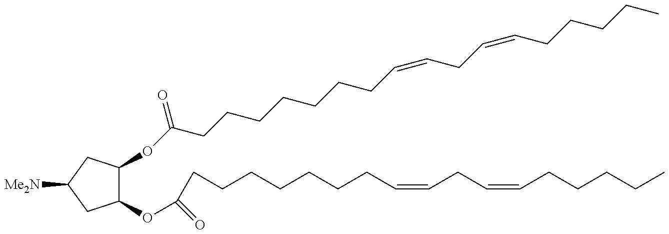 [SMILES: CCCCC/C=C\C/C=C\CCCCCCCC(=O)O[C@H]1C[C@@H](N(C)C)C[C@H]1OC(=O)CCCCCCC/C=C\C/C=C\CCCCC]

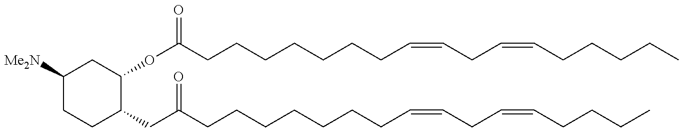 [SMILES: CCCC/C=C\C/C=C\CCCCCCCC(=O)C[C@@H]1CC[C@@H](N(C)C)C[C@@H]1OC(=O)CCCCCCC/C=C\C/C=C\CCCCC]

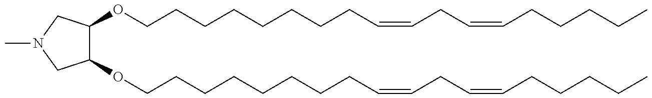 [SMILES: CCCCC/C=C\C/C=C\CCCCCCCCO[C@H]1CN(C)C[C@H]1OCCCCCCCC/C=C\C/C=C\CCCCC]

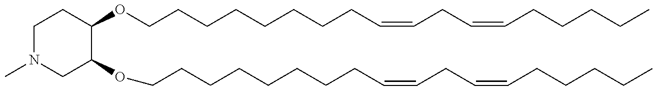 [SMILES: CCCCC/C=C\C/C=C\CCCCCCCCO[C@H]1CN(C)CC[C@H]1OCCCCCCCC/C=C\C/C=C\CCCCC]

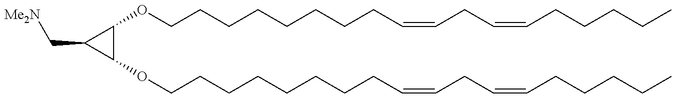 [SMILES: CCCCC/C=C\C/C=C\CCCCCCCCO[C@@H]1[C@@H](CN(C)C)[C@@H]1OCCCCCCCC/C=C\C/C=C\CCCCC]

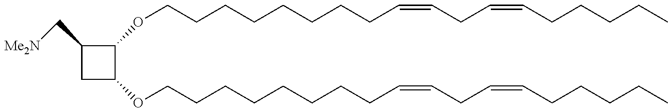 [SMILES: CCCCC/C=C\C/C=C\CCCCCCCCO[C@H]1[C@H](CN(C)C)C[C@H]1OCCCCCCCC/C=C\C/C=C\CCCCC]

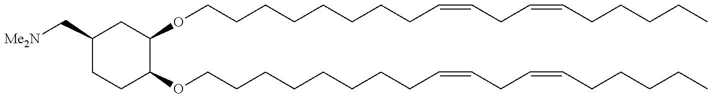 [SMILES: CCCCC/C=C\C/C=C\CCCCCCCCO[C@H]1CC[C@@H](CN(C)C)C[C@H]1OCCCCCCCC/C=C\C/C=C\CCCCC]

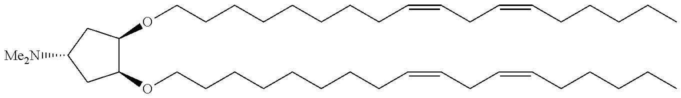 [SMILES: CCCCC/C=C\C/C=C\CCCCCCCCO[C@H]1C[C@H](N(C)C)C[C@H]1OCCCCCCCC/C=C\C/C=C\CCCCC]

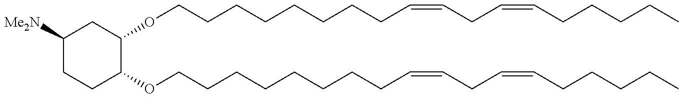 [SMILES: CCCCC/C=C\C/C=C\CCCCCCCCO[C@H]1C[C@H](N(C)C)CC[C@H]1OCCCCCCCC/C=C\C/C=C\CCCCC]

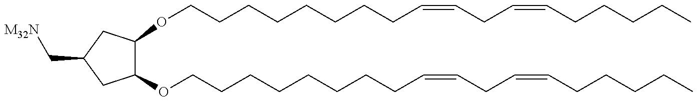

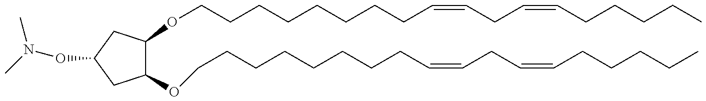 [SMILES: CCCCC/C=C\C/C=C\CCCCCCCCO[C@H]1C[C@H](ON(C)C)C[C@H]1OCCCCCCCC/C=C\C/C=C\CCCCC]

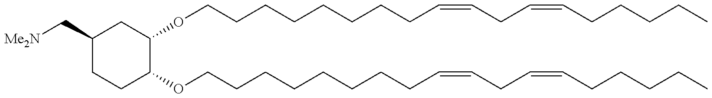 [SMILES: CCCCC/C=C\C/C=C\CCCCCCCCO[C@H]1C[C@H](CN(C)C)CC[C@H]1OCCCCCCCC/C=C\C/C=C\CCCCC]

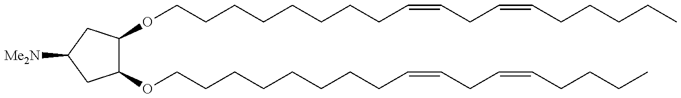 [SMILES: CCCC/C=C\C/C=C\CCCCCCCCO[C@H]1C[C@@H](N(C)C)C[C@H]1OCCCCCCCC/C=C\C/C=C\CCCCC]

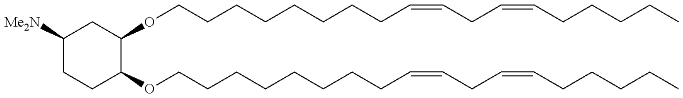 [SMILES: CCCCC/C=C\C/C=C\CCCCCCCCO[C@H]1CC[C@@H](N(C)C)C[C@H]1OCCCCCCCC/C=C\C/C=C\CCCCC]

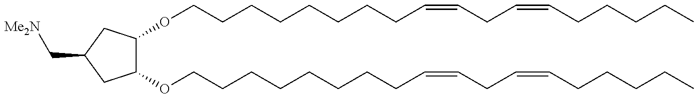 [SMILES: CCCCC/C=C\C/C=C\CCCCCCCCO[C@H]1C[C@H](CN(C)C)C[C@H]1OCCCCCCCC/C=C\C/C=C\CCCCC]

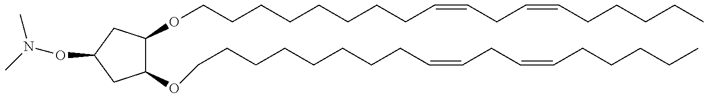 [SMILES: CCCCC/C=C\C/C=C\CCCCCCCCO[C@H]1C[C@@H](ON(C)C)C[C@H]1OCCCCCCCC/C=C\C/C=C\CCCCC]

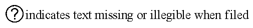

In some embodiments, the transfer vehicle comprises Lipid A, Lipid B, Lipid C, and/or Lipid D. In some embodiments, inclusion of Lipid A, Lipid B, Lipid C, and/or Lipid D improves encapsulation and/or endosomal escape. In some embodiments, Lipid A, Lipid B, Lipid C, and/or Lipid D are described in international patent application PCT/US2017/028981.

In some embodiments, an ionizable lipid is Lipid A, which is (9Z,12Z)-3-((4,4-bis(octyloxy)butanoyl)oxy)-2-((((3-(diethylamino)propoxy)carbonyl)oxy)methyl)propyl octadeca9,12-dienoate, also called 3-((4,44bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

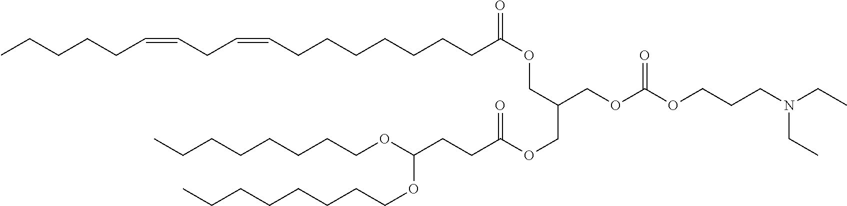 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86), incorporated by reference in its entirety.

In some embodiments, an ionizable lipid is Lipid B, which is ((5-((dimethylamino)methyl)-1,3-phenylene)bis(oxy))bis(octane-8,1-diyl)bis(decanoate). Lipid B can be depicted as:

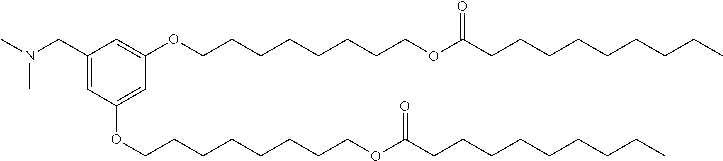 [SMILES: CCCCCCCCCC(=O)OCCCCCCCCOc1cc(CN(C)C)cc(OCCCCCCCCOC(=O)CCCCCCCCC)c1]

Lipid B may be synthesized according to WO2014/136086 (e.g., pp. 107-09), incorporated by reference in its entirety.

In some embodiments, an ionizable lipid is Lipid C, which is 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate). Lipid C can be depicted as:

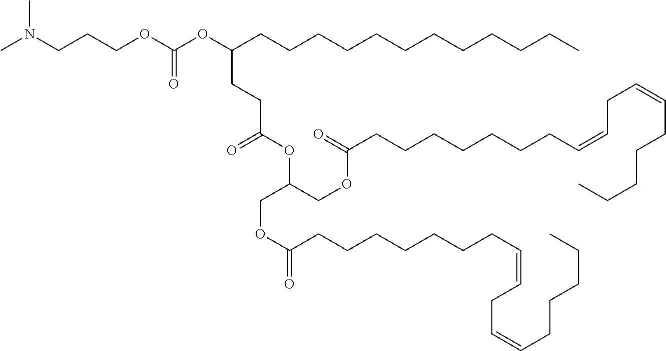 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

In some embodiments, an ionizable lipid is Lipid D, which is 3-(((3-(dimethylamino)propoxy)carbonyl)oxy)-13-(octanoyloxy)tridecyl 3-octylundecanoate. Lipid D can be depicted as:

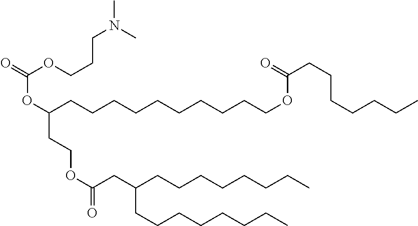 [SMILES: CCCCCCCCC(CCCCCCCC)CC(=O)OCCC(CCCCCCCCCCOC(=O)CCCCCCC)OC(=O)OCCCN(C)C]

Lipid C and Lipid D may be synthesized according to WO2015/095340, incorporated by reference in its entirety.

In some embodiments, an ionizable lipid is described in US patent publication number 20190321489. In some embodiments, an ionizable lipid is described in international patent publication WO 2010/053572, incorporated herein by reference. In some embodiments, an ionizable lipid is C12-200, described at paragraph [00225] of WO 2010/053572.

Several ionizable lipids have been described in the literature, many of which are commercially available. In certain embodiments, such ionizable lipids are included in the transfer vehicles described herein. In some embodiments, the ionizable lipid N-[1-(2,3-dioleyloxy)propyl]-N,N,N-trimethylammonium chloride or “DOTMA” is used. (Felgner et al. Proc. Nat'l Acad. Sci. 84, 7413 (1987); U.S. Pat. No. 4,897,355). DOTMA can be formulated alone or can be combined with a neutral lipid, dioleoylphosphatidylethanolamine or “DOPE” or other cationic or non-cationic lipids into a lipid nanoparticle. Other suitable cationic lipids include, for example, ionizable cationic lipids as described in U.S. provisional patent application 61/617,468, filed Mar. 29, 2012 (incorporated herein by reference), such as, e.g., (15Z,18Z)—N,N-dimethyl-6-(9Z,12Z)-octadeca-9,12-dien-1-yl)tetracosa-15,18-dien-1-amine (HGT5000), (15Z,18Z)—N,N-dimethyl-6-((9Z,12Z)-octadeca-9,12-dien yl)tetracosa-4,15,18-trien-1-amine (HGT5001), and (15Z,18Z)—N,N-dimethyl-6-((9Z,12Z)-octadeca-9,12-dien-1-yl)tetracosa-5,15,18-trien-1-amine (HGT5002), C12-200 (described in WO 2010/053572), 2-(2,2-di((9Z,12Z)-octadeca-9,12-dien-1-yl)-1,3-dioxolan-4-yl)-N,N-dimethylethanamine (DLinKC2-DMA)) (See, WO 2010/042877; Semple et al., Nature Biotech. 28:172-176 (2010)), 2-(2,2-di((9Z,2Z)-octadeca-9,12-dien-1-yl)-1,3-dioxolan-4-yl)-N,N-dimethylethanamine (DLin-KC2-DMA), (3 S,10R,13R,17R)-10,13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate (ICE), (15Z,18Z)—N,N-dimethyl-6-(9Z,12Z)-octadeca-9,12-dien-1-yl)tetracosa-15,18-dien-1-amine (HGT5000), (15Z,18Z)—N,N-dimethyl-6-((9Z,12Z)-octadeca-9,12-dien-1-yl)tetracosa-4,15,18-trien-1-amine (HGT5001), (15Z,18 Z)—N,N-dimethyl-6-((9Z,12Z)-octadeca-9,12-dien-1-yl)tetracosa-5,15,18-trien-1-amine (HGT5002), 5-carboxyspermylglycine-dioctadecylamide (DOGS), 2,3-dioleyloxy-N-[2(spermine-carboxamido)ethyl]-N,N-dimethyl-1-propanaminium (DOSPA) (Behr et al. Proc. Nat.'l Acad. Sci. 86, 6982 (1989); U.S. Pat. Nos. 5,171,678; 5,334,761), 1,2-Dioleoyl-3-Dimethylammonium-Propane (DODAP), 1,2-Dioleoyl-3-Trimethylammonium-Propane or (DOTAP). Contemplated ionizable lipids also include 1,2-distcaryloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), N-dioleyl-N,N-dimethylammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylamninopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl[1,3]-dioxolane (DLin-K-XTC2-DMA) or GL67, or mixtures thereof. (Heyes, J., et al., J Controlled Release 107: 276-287 (2005); Morrissey, D V., et al., Nat. Biotechnol. 23(8): 1003-1007 (2005); PCT Publication WO2005/121348A1). The use of cholesterol-based ionizable lipids to formulate the transfer vehicles (e.g., lipid nanoparticles) is also contemplated by the present invention. Such cholesterol-based ionizable lipids can be used, either alone or in combination with other lipids. Suitable cholesterol-based ionizable lipids include, for example, DC-Cholesterol (N,N-dimethyl-N-ethylcarboxamidocholesterol), and 1,4-bis(3-N-oleylamino-propyl)piperazine (Gao, et al., Biochem. Biophys. Res. Comm. 179, 280 (1991); Wolf et al. BioTechniques 23, 139 (1997); U.S. Pat. No. 5,744,335).

Also contemplated are cationic lipids such as dialkylamino-based, imidazole-based, and guanidinium-based lipids. For example, also contemplated is the use of the ionizable lipid (3S,10R, 13R, 17R)-10,13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate (ICE), as disclosed in International Application No. PCT/US2010/058457, incorporated herein by reference.

Also contemplated are ionizable lipids such as the dialkylamino-based, imidazole-based, and guanidinium-based lipids. For example, certain embodiments are directed to a composition comprising one or more imidazole-based ionizable lipids, for example, the imidazole cholesterol ester or “ICE” lipid, (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, as represented by structure (XIII) below. In an embodiment, a transfer vehicle for delivery of circRNA may comprise one or more imidazole-based ionizable lipids, for example, the imidazole cholesterol ester or “ICE” lipid (3S, 10R, 13R, 17R)-10, 13-dimethyl-17-((R)-6-methylheptan-2-yl)-2, 3, 4, 7, 9, 10, 11, 12, 13, 14, 15, 16, 17-tetradecahydro-1H-cyclopenta[a]phenanthren-3-yl 3-(1H-imidazol-4-yl)propanoate, as represented by structure (XIII).

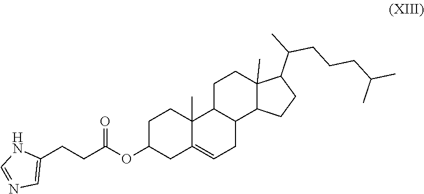 [SMILES: CC(C)CCCC(C)C1CCC2C3CC=C4CC(OC(=O)CCc5cnc[nH]5)CCC4(C)C3CCC12C]

Without wishing to be bound by a particular theory, it is believed that the fusogenicity of the imidazole-based cationic lipid ICE is related to the endosomal disruption which is facilitated by the imidazole group, which has a lower pKa relative to traditional ionizable lipids. The endosomal disruption in turn promotes osmotic swelling and the disruption of the liposomal membrane, followed by the transfection or intracellular release of the nucleic acid(s) contents loaded therein into the target cell.

The imidazole-based ionizable lipids are also characterized by their reduced toxicity relative to other ionizable lipids.

In some embodiments, an ionizable lipid is described by US patent publication number 20190314284. In certain embodiments, the an ionizable lipid is described by structure 3, 4, 5, 6, 7, 8, 9, or 10 (e.g., HGT4001, HGT4002, HGT4003, HGT4004 and/or HGT4005). In certain embodiments, the one or more cleavable functional groups (e.g., a disulfide) allow, for example, a hydrophilic functional head-group to dissociate from a lipophilic functional tail-group of the compound (e.g., upon exposure to oxidative, reducing or acidic conditions), thereby facilitating a phase transition in the lipid bilayer of the one or more target cells. For example, when a transfer vehicle (e.g., a lipid nanoparticle) comprises one or more of the lipids of structures 3-10, the phase transition in the lipid bilayer of the one or more target cells facilitates the delivery of the circRNA into the one or more target cells.

In certain embodiments, the ionizable lipid is described by structure (XIV),

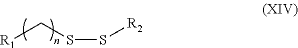

wherein:

R₁ is selected from the group consisting of imidazole, guanidinium, amino, imine, enamine, an optionally-substituted alkyl amino (e.g., an alkyl amino such as dimethylamino) and pyridyl;

R₂ is selected from the group consisting of structure XV and structure XVI;

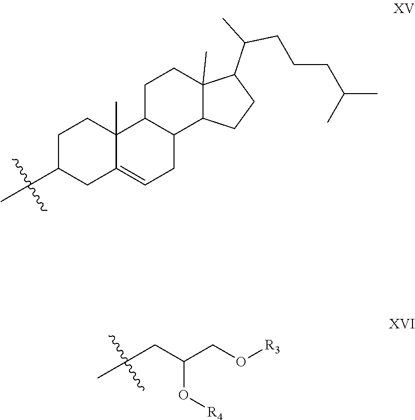

wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In certain embodiments, R₃ and R₄ are each an optionally substituted, polyunsaturated Cis alkyl, while in other embodiments R₃ and R₄ are each an unsubstituted, polyunsaturated Cis alkyl. In certain embodiments, one or more of R₃ and R₄ are (9Z,12Z)-octadeca-9,12-dien.

Also disclosed herein are pharmaceutical compositions that comprise the compound of structure XIV, wherein R₁ is selected from the group consisting of imidazole, guanidinium, amino, imine, enamine, an optionally-substituted alkyl amino (e.g., an alkyl amino such as dimethylamino) and pyridyl; wherein R₂ is structure XV; and wherein n is zero or any positive integer. Further disclosed herein are pharmaceutical compositions comprising the compound of structure XIV, wherein R₁ is selected from the group consisting of imidazole, guanidinium, amino, imine, enamine, an optionally-substituted alkyl amino (e.g., an alkyl amino such as dimethylamino) and pyridyl; wherein R₂ is structure XVI; wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer. In certain embodiments. R₃ and R₄ are each an optionally substituted, polyunsaturated Cis alkyl, while in other embodiments R₃ and R₄ are each an unsubstituted, polyunsaturated Cis alkyl (e.g., octadeca-9,12-dien).

In certain embodiments, the R₁ group or head-group is a polar or hydrophilic group (e.g., one or more of the imidazole, guanidinium and amino groups) and is bound to the R₂ lipid group by way of the disulfide (S—S) cleavable linker group, for example as depicted in structure XIV. Other contemplated cleavable linker groups may include compositions that comprise one or more disulfide (S—S) linker group bound (e.g., covalently bound) to, for example an alkyl group (e.g., C₁ to C₁₀ alkyl). In certain embodiments, the R₁ group is covalently bound to the cleavable linker group by way of a C₁-C₂₀ alkyl group (e.g., where n is one to twenty), or alternatively may be directly bound to the cleavable linker group (e.g., where n is zero). In certain embodiments, the disulfide linker group is cleavable in vitro and/or in vivo (e.g., enzymatically cleavable or cleavable upon exposure to acidic or reducing conditions).

In certain embodiments, the inventions relate to the compound 5-(((10,13-dimethyl-17-(6-methylheptan-2-yl)-2,3,4,7,8,9,10,11,12,13,14,15,16,17-tetradecahydro-1H-cyclopenta[a]phenanthren-3-yl)disulfanyl)methyl)-1H-imidazole, having structure XVII (referred to herein as “HGT4001”).

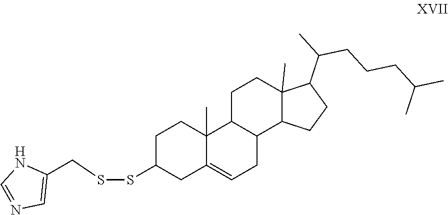 [SMILES: CC(C)CCCC(C)C1CCC2C3CC=C4CC(SSCc5cnc[nH]5)CCC4(C)C3CCC12C]

In certain embodiments, the inventions relate to the compound 1-(2-(((3 S,10R,13R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfanyl)ethyl)guanidine, having structure XVIII (referred to herein as “HGT4002”).

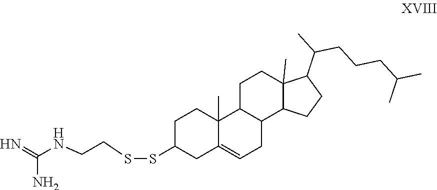 [SMILES: CC(C)CCCC(C)C1CCC2C3CC=C4CC(SSCCNC(=N)N)CCC4(C)C3CCC12C]

In certain embodiments, the inventions relate to the compound 2-((2,3-Bis((9Z,12Z)-octadeca-9,12-dien-1-yloxy)propyl)disulfanyl)-N,N-dimethylethanamine, having structure XIX (referred to herein as “HGT4003”).

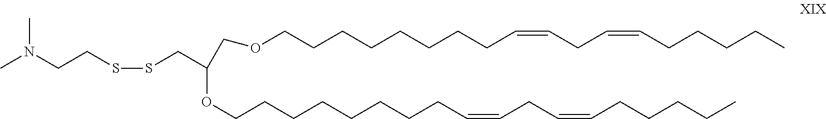 [SMILES: CCCCC/C=C\C/C=C\CCCCCCCCOCC(CSSCCN(C)C)OCCCCCCCC/C=C\C/C=C\CCCCC]

In other embodiments, the inventions relate to the compound 5-(((2,3-bis((9Z,12Z)-octadeca-9,12-dien-1-yloxy)propyl)disulfanyl)methyl)-1H-imidazole having the structure of structure XX (referred to herein as “HGT4004”).

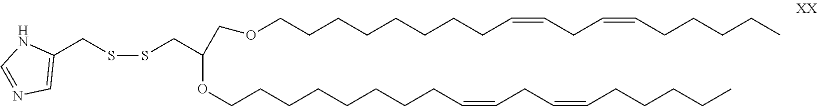 [SMILES: CCCCC/C=C\C/C=C\CCCCCCCCOCC(CSSCc1cnc[nH]1)OCCCCCCCC/C=C\C/C=C\CCCCC]

In still other embodiments, the inventions relate to the compound 1-(((2,3-bis((9Z,12Z)-octadeca-9,12-dien-1-yloxy)propyl)disulfanyl)methyl)guanidine having structure XXI (referred to herein as “HGT4005”).

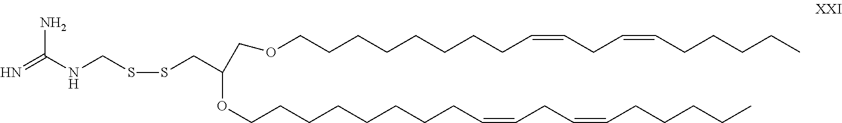 [SMILES: CCCCC/C=C\C/C=C\CCCCCCCCOCC(CSSCNC(=N)N)OCCCCCCCC/C=C\C/C=C\CCCCC]

In certain embodiments, the compounds described as structures 3-10 are ionizable lipids.

The compounds, and in particular the imidazole-based compounds described as structures 3-8 (e.g., HGT4001 and HGT4004), are characterized by their reduced toxicity, in particular relative to traditional ionizable lipids. In some embodiments, the transfer vehicles described herein comprise one or more imidazole-based ionizable lipid compounds such that the relative concentration of other more toxic ionizable lipids in such pharmaceutical or liposomal composition may be reduced or otherwise eliminated.

The ionizable lipids include those disclosed in international patent application PCT/US2019/025246, and US patent publications 2017/0190661 and 2017/0114010, incorporated herein by reference in their entirety. The ionizable lipids may include a lipid selected from the following tables 12, 13, 14, or 15.

TABLE 12

| | |
|---|---|
| 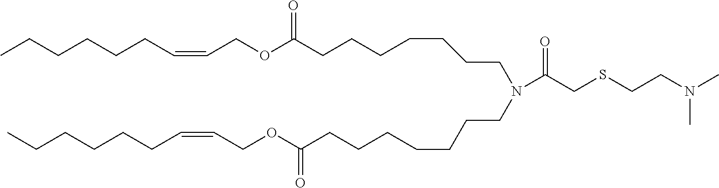 [SMILES: CCCCCC/C=C\COC(=O)CCCCCCCN(CCCCCCCC(=O)OC/C=C\CCCCCC)C(=O)CSCCN(C)C] | ATX-001 |
| 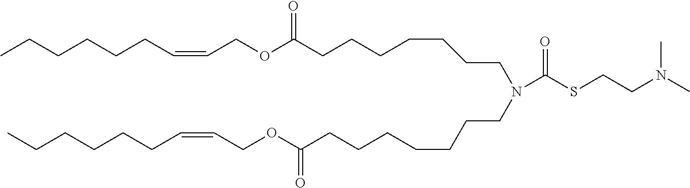 [SMILES: CCCCCC/C=C\COC(=O)CCCCCCCN(CCCCCCCC(=O)OC/C=C\CCCCCC)C(=O)SCCN(C)C] | ATX-002 |
| 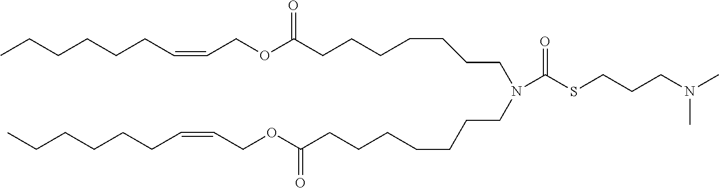 [SMILES: CCCCCC/C=C\COC(=O)CCCCCCCN(CCCCCCCC(=O)OC/C=C\CCCCCC)C(=O)SCCCN(C)C] | ATX-003 |
| 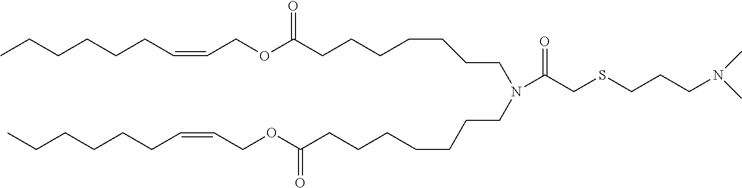 [SMILES: CCCCCC/C=C\COC(=O)CCCCCCCN(CCCCCCCC(=O)OC/C=C\CCCCCC)C(=O)CSCCCN(C)C] | ATX-004 |
| 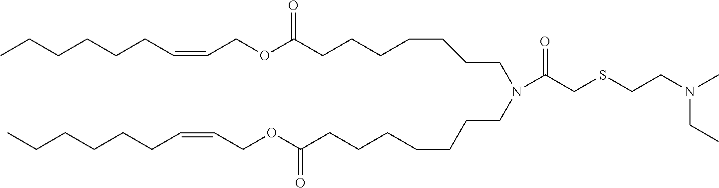 [SMILES: CCCCCC/C=C\COC(=O)CCCCCCCN(CCCCCCCC(=O)OC/C=C\CCCCCC)C(=O)CSCCN(C)CC] | ATX-005 |
| 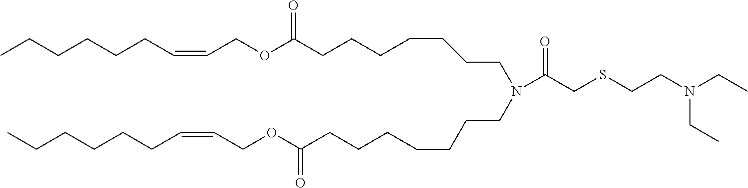 [SMILES: CCCCCC/C=C\COC(=O)CCCCCCCN(CCCCCCCC(=O)OC/C=C\CCCCCC)C(=O)CSCCN(CC)CC] | ATX-006 |
| 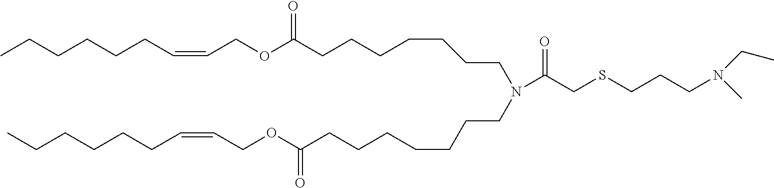 [SMILES: CCCCCC/C=C\COC(=O)CCCCCCCN(CCCCCCCC(=O)OC/C=C\CCCCCC)C(=O)CSCCCN(C)CC] | ATX-007 |
| 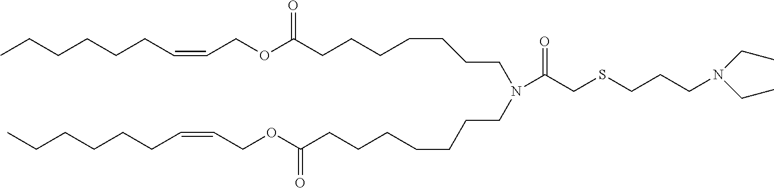 [SMILES: CCCCCC/C=C\COC(=O)CCCCCCCN(CCCCCCCC(=O)OC/C=C\CCCCCC)C(=O)CSCCCN(CC)CC] | ATX-008 |
| 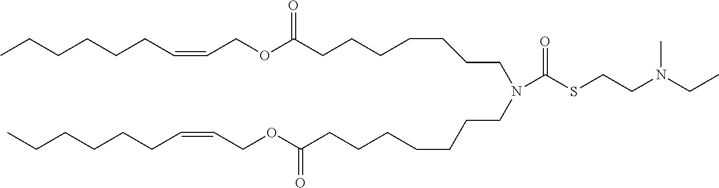 [SMILES: CCCCCC/C=C\COC(=O)CCCCCCCN(CCCCCCCC(=O)OC/C=C\CCCCCC)C(=O)SCCN(C)CC] | ATX-009 |
| 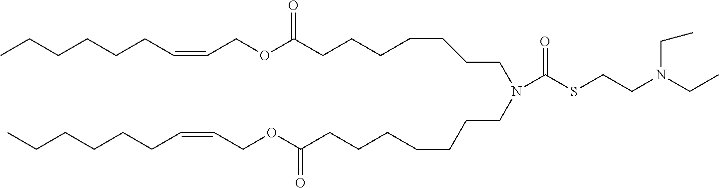 [SMILES: CCCCCC/C=C\COC(=O)CCCCCCCN(CCCCCCCC(=O)OC/C=C\CCCCCC)C(=O)SCCN(CC)CC] | ATX-010 |
| 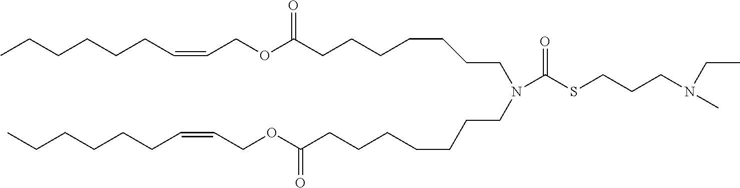 [SMILES: CCCCCC/C=C\COC(=O)CCCCCCCN(CCCCCCCC(=O)OC/C=C\CCCCCC)C(=O)SCCCN(C)CC] | ATX-011 |
| 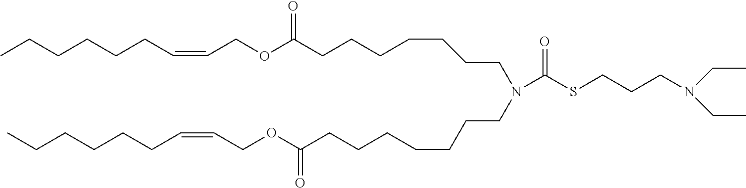 [SMILES: CCCCCC/C=C\COC(=O)CCCCCCCN(CCCCCCCC(=O)OC/C=C\CCCCCC)C(=O)SCCCN(CC)CC] | ATX-012 |
| 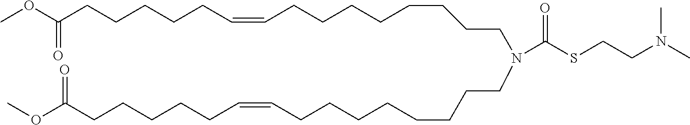 | ATX-013 |
| 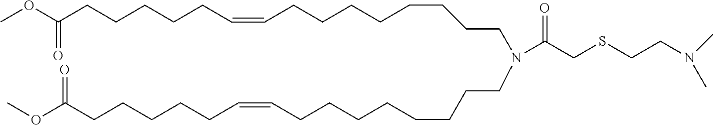 | ATX-014 |
| 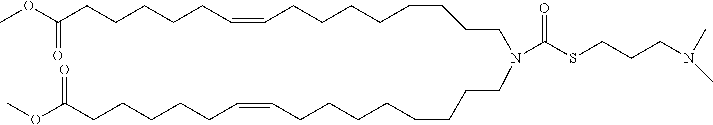 | ATX-015 |
| 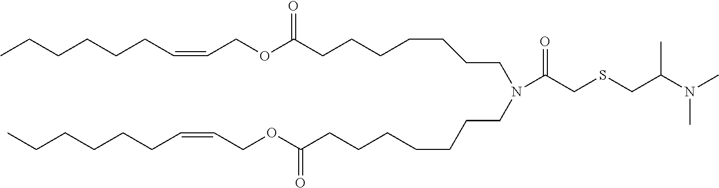 [SMILES: CCCCCC/C=C\COC(=O)CCCCCCCN(CCCCCCCC(=O)OC/C=C\CCCCCC)C(=O)CSCC(C)N(C)C] | ATX-016 |
| 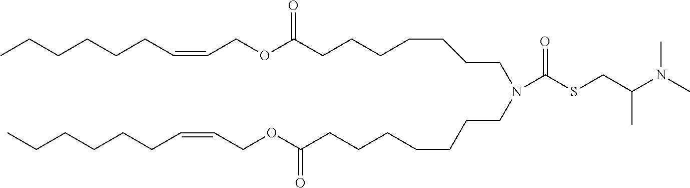 [SMILES: CCCCCC/C=C\COC(=O)CCCCCCCN(CCCCCCCC(=O)OC/C=C\CCCCCC)C(=O)SCC(C)N(C)C] | ATX-017 |
| 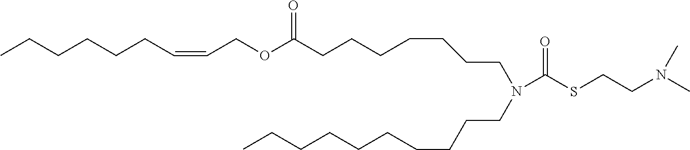 [SMILES: CCCCCC/C=C\COC(=O)CCCCCCCN(CCCCCCCCCC)C(=O)SCCN(C)C] | ATX-018 |
| 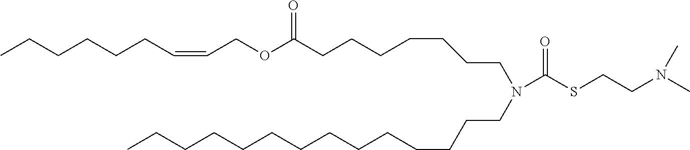 [SMILES: CCCCCC/C=C\COC(=O)CCCCCCCN(CCCCCCCCCCCCCC)C(=O)SCCN(C)C] | ATX-019 |
| 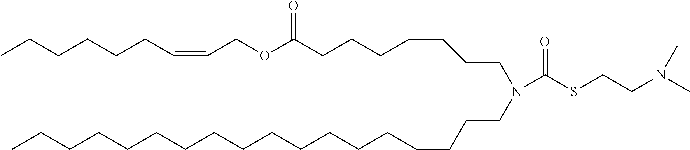 [SMILES: CCCCCC/C=C\COC(=O)CCCCCCCN(CCCCCCCCCCCCCCCCCC)C(=O)SCCN(C)C] | ATX-020 |
| 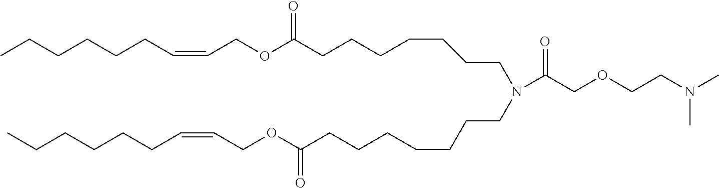 [SMILES: CCCCCC/C=C\COC(=O)CCCCCCCN(CCCCCCCC(=O)OC/C=C\CCCCCC)C(=O)COCCN(C)C] | ATX-021 |
| 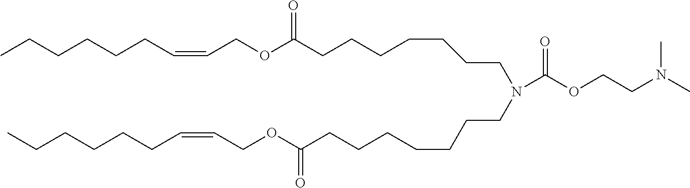 [SMILES: CCCCCC/C=C\COC(=O)CCCCCCCN(CCCCCCCC(=O)OC/C=C\CCCCCC)C(=O)OCCN(C)C] | ATX-022 |
| 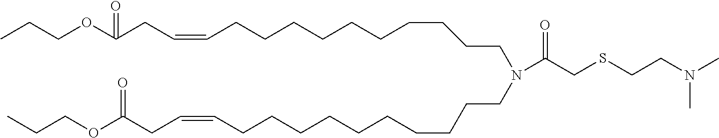 [SMILES: CCCOC(=O)C/C=C\CCCCCCCCCCN(CCCCCCCCCC/C=C\CC(=O)OCCC)C(=O)CSCCN(C)C] | ATX-023 |
| 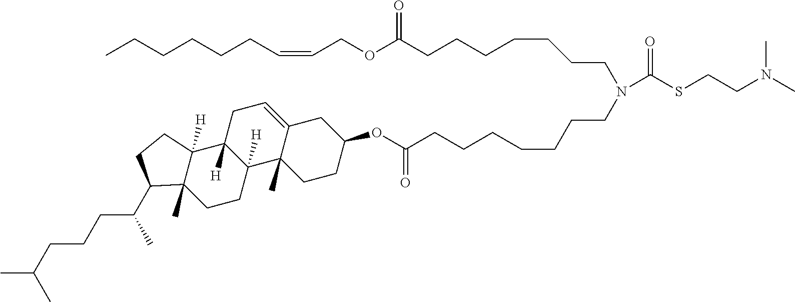 | ATX-024 |
| 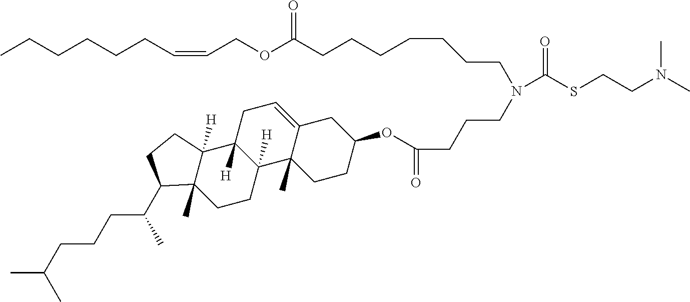 | ATX-025 |
| 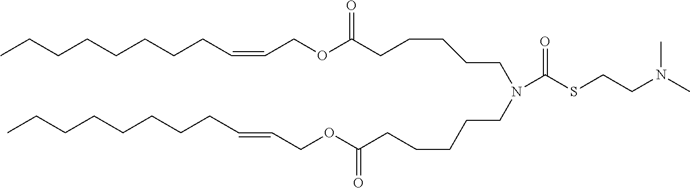 [SMILES: CCCCCCCC/C=C\COC(=O)CCCCCN(CCCCCC(=O)OC/C=C/CCCCCCCC)C(=O)SCCN(C)C] | ATX-026 |
| 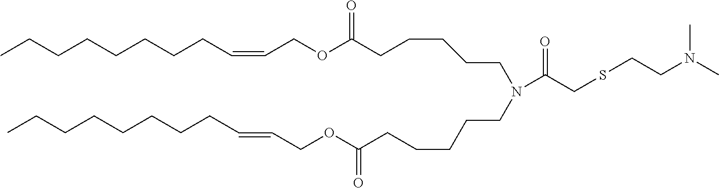 [SMILES: CCCCCCCC/C=C\COC(=O)CCCCCN(CCCCCC(=O)OC/C=C/CCCCCCCC)C(=O)CSCCN(C)C] | ATX-027 |
| 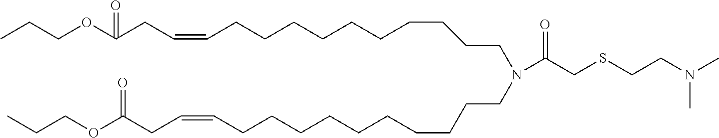 [SMILES: CCCOC(=O)C/C=C\CCCCCCCCCCN(CCCCCCCCCC/C=C\CC(=O)OCCC)C(=O)CSCCN(C)C] | ATX-028 |
| 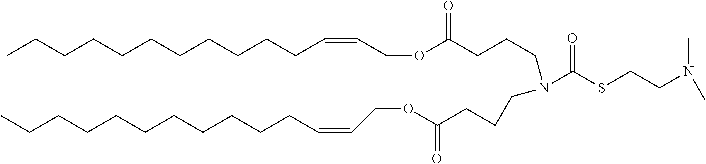 [SMILES: CCCCCCCCCCC/C=C\COC(=O)CCCN(CCCC(=O)OC/C=C\CCCCCCCCCCC)C(=O)SCCN(C)C] | ATX-029 |
| 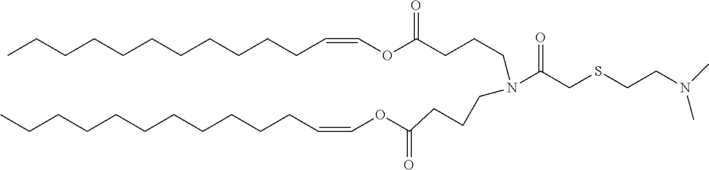 [SMILES: CCCCCCCCCCC/C=C\OC(=O)CCCN(CCCC(=O)O/C=C\CCCCCCCCCCC)C(=O)CSCCN(C)C] | ATX-030 |
| 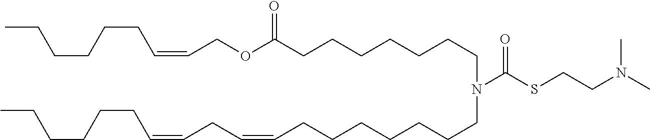 [SMILES: CCCCCC/C=C\C/C=C\CCCCCCCN(CCCCCCCC(=O)OC/C=C\CCCCCC)C(=O)SCCN(C)C] | ATX-031 |
| 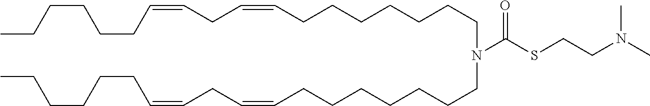 [SMILES: CCCCCC/C=C\C/C=C\CCCCCCCN(CCCCCCC/C=C\C/C=C\CCCCCC)C(=O)SCCN(C)C] | ATX-032 |

TABLE 13

| | |
|---|---|
| 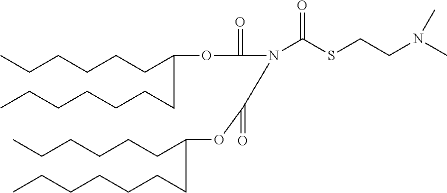 [SMILES: CCCCCCCC(CCCCCC)OC(=O)N(C(=O)OC(CCCCCC)CCCCCCC)C(=O)SCCN(C)C] | ATX-B-1 |
| 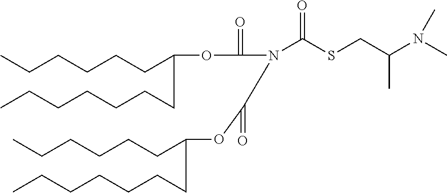 [SMILES: CCCCCCCC(CCCCCC)OC(=O)N(C(=O)OC(CCCCCC)CCCCCCC)C(=O)SCC(C)N(C)C] | ATX-B-2 |
| 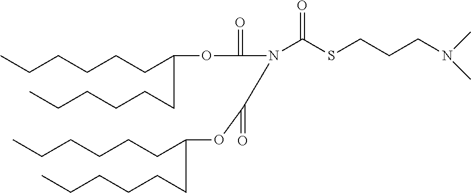 [SMILES: CCCCCCC(CCCCCC)OC(=O)N(C(=O)OC(CCCCCC)CCCCCC)C(=O)SCCCN(C)C] | ATX-B-3 |
| 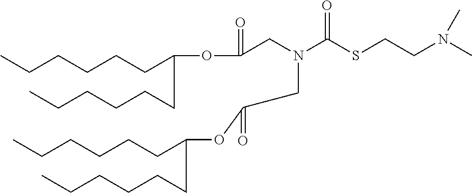 [SMILES: CCCCCCC(CCCCCC)OC(=O)CN(CC(=O)OC(CCCCCC)CCCCCC)C(=O)SCCN(C)C] | ATX-B-4 |
| 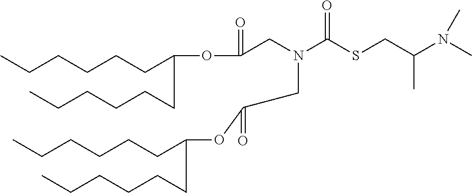 [SMILES: CCCCCCC(CCCCCC)OC(=O)CN(CC(=O)OC(CCCCCC)CCCCCC)C(=O)SCC(C)N(C)C] | ATX-B-5 |
| 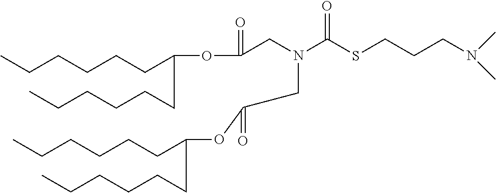 [SMILES: CCCCCCC(CCCCCC)OC(=O)CN(CC(=O)OC(CCCCCC)CCCCCC)C(=O)SCCCN(C)C] | ATX-B-6 |
| 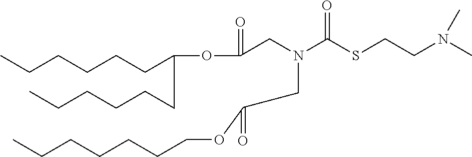 [SMILES: CCCCCCCOC(=O)CN(CC(=O)OC(CCCCCC)CCCCCC)C(=O)SCCN(C)C] | ATX-B-7 |
| 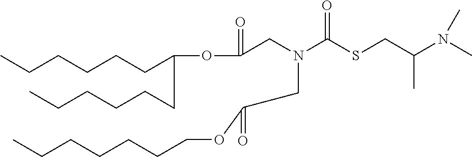 [SMILES: CCCCCCCOC(=O)CN(CC(=O)OC(CCCCCC)CCCCCC)C(=O)SCC(C)N(C)C] | ATX-B-8 |
| 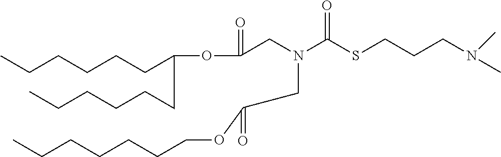 [SMILES: CCCCCCCOC(=O)CN(CC(=O)OC(CCCCCC)CCCCCC)C(=O)SCCCN(C)C] | ATX-B-9 |
| 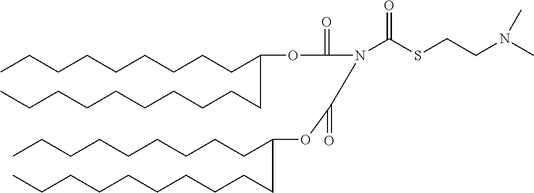 [SMILES: CCCCCCCCCCC(CCCCCCCCC)OC(=O)N(C(=O)OC(CCCCCCCCC)CCCCCCCCCC)C(=O)SCCN(C)C] | ATX-B-10 |
| 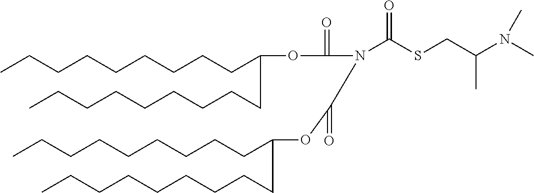 [SMILES: CCCCCCCCCC(CCCCCCCCC)OC(=O)N(C(=O)OC(CCCCCCCCC)CCCCCCCCC)C(=O)SCC(C)N(C)C] | ATX-B-11 |
| 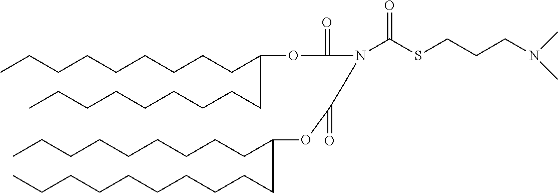 [SMILES: CCCCCCCCCCC(CCCCCCCCC)OC(=O)N(C(=O)OC(CCCCCCCCC)CCCCCCCCC)C(=O)SCCCN(C)C] | ATX-B-12 |

TABLE 14

| Compound | ATX-# |
|---|---|
| 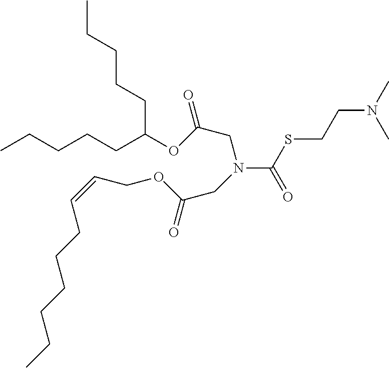 [SMILES: CCCCCC/C=C\COC(=O)CN(CC(=O)OC(CCCCC)CCCCC)C(=O)SCCN(C)C] | 0063 |
| 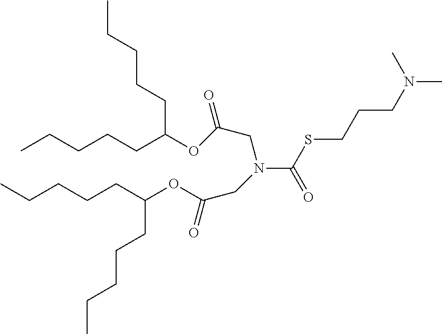 [SMILES: CCCCCC(CCCCC)OC(=O)CN(CC(=O)OC(CCCCC)CCCCC)C(=O)SCCCN(C)C] | 0130 |
| 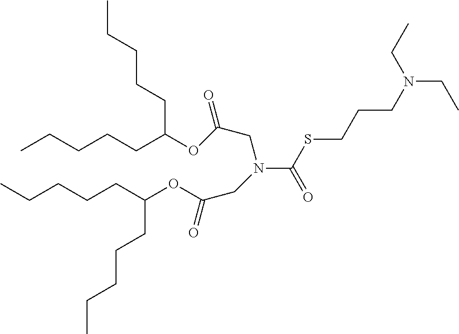 [SMILES: CCCCCC(CCCCC)OC(=O)CN(CC(=O)OC(CCCCC)CCCCC)C(=O)SCCCN(CC)CC] | 0131 |
| 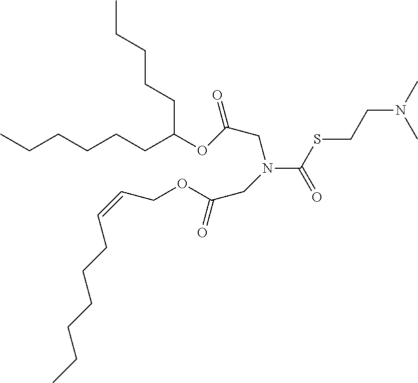 [SMILES: CCCCCC/C=C\COC(=O)CN(CC(=O)OC(CCCCC)CCCCCC)C(=O)SCCN(C)C] | 0044 |
| 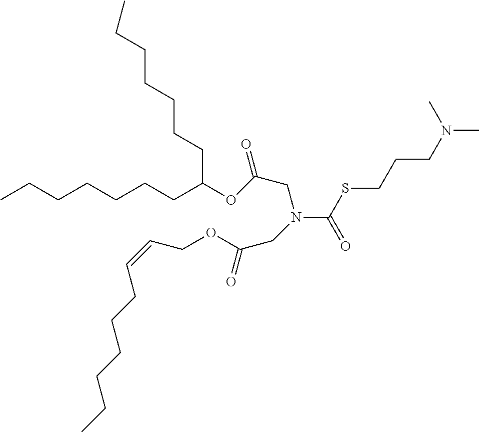 [SMILES: CCCCCC/C=C\COC(=O)CN(CC(=O)OC(CCCCCCC)CCCCCCC)C(=O)SCCCN(C)C] | 0111 |
| 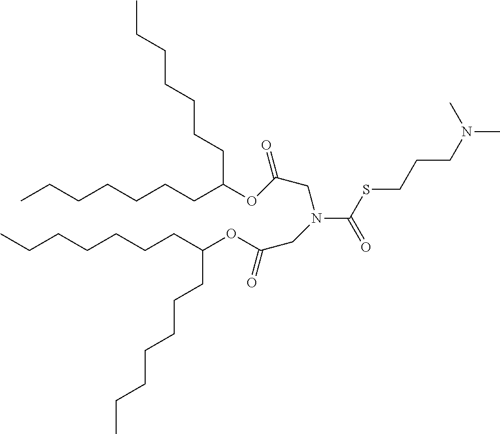 [SMILES: CCCCCCCC(CCCCCCC)OC(=O)CN(CC(=O)OC(CCCCCCC)CCCCCCC)C(=O)SCCCN(C)C] | 0132 |
| 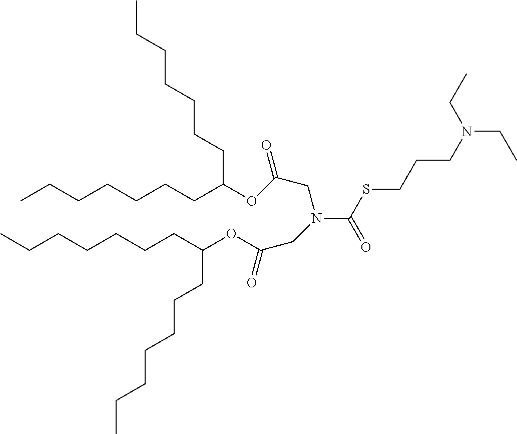 [SMILES: CCCCCCCC(CCCCCCC)OC(=O)CN(CC(=O)OC(CCCCCCC)CCCCCCC)C(=O)SCCCN(CC)CC] | 0134 |
| 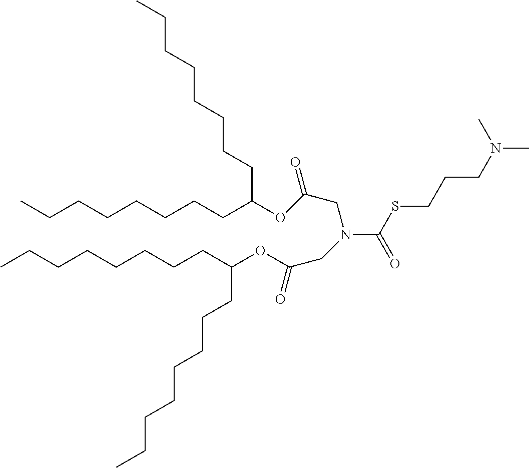 [SMILES: CCCCCCCCC(CCCCCCCC)OC(=O)CN(CC(=O)OC(CCCCCCCC)CCCCCCCC)C(=O)SCCCN(C)C] | 0133 |
| 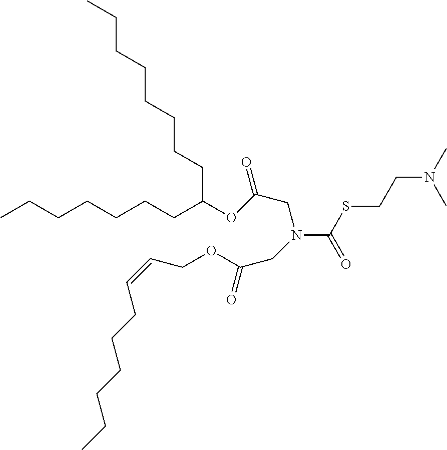 [SMILES: CCCCCC/C=C\COC(=O)CN(CC(=O)OC(CCCCCCC)CCCCCCCC)C(=O)SCCN(C)C] | 0064 |
| 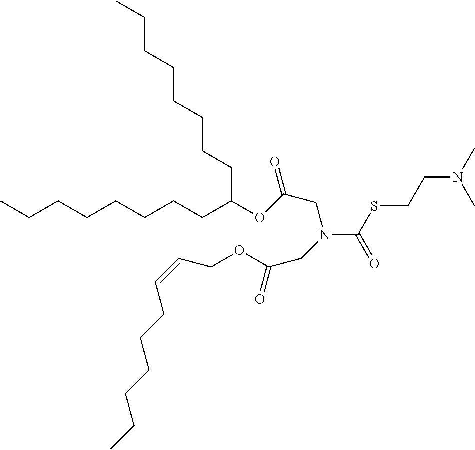 [SMILES: CCCCCC/C=C\COC(=O)CN(CC(=O)OC(CCCCCCCC)CCCCCCCC)C(=O)SCCN(C)C] | 0061 |
| 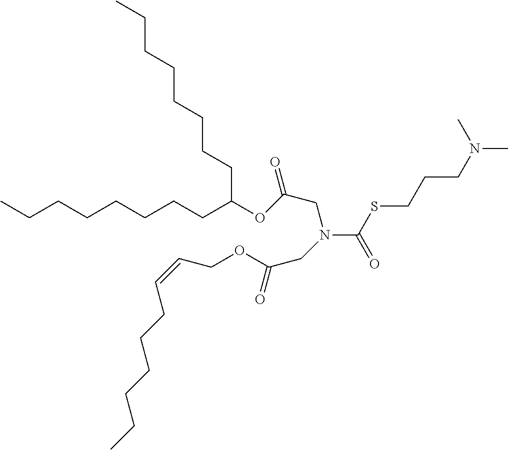 [SMILES: CCCCCC/C=C\COC(=O)CN(CC(=O)OC(CCCCCCCC)CCCCCCCC)C(=O)SCCCN(C)C] | 0100 |
| 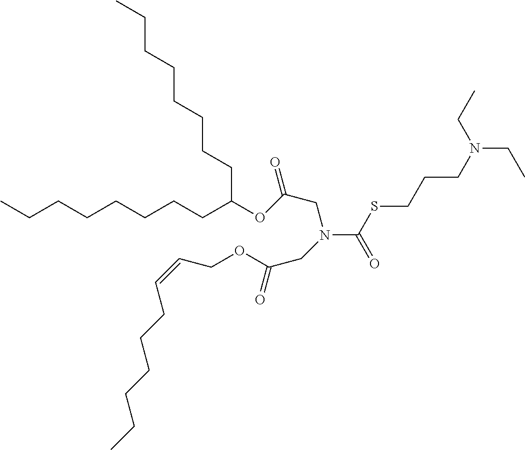 [SMILES: CCCCCC/C=C\COC(=O)CN(CC(=O)OC(CCCCCCCC)CCCCCCCC)C(=O)SCCCN(CC)CC] | 0117 |
| 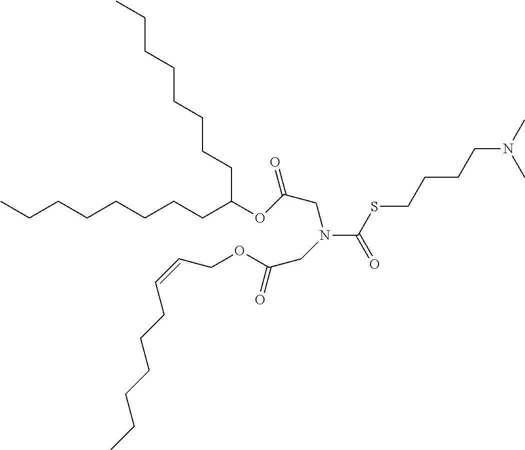 [SMILES: CCCCCC/C=C\COC(=O)CN(CC(=O)OC(CCCCCCCC)CCCCCCCC)C(=O)SCCCCN(C)C] | 0114 |
| 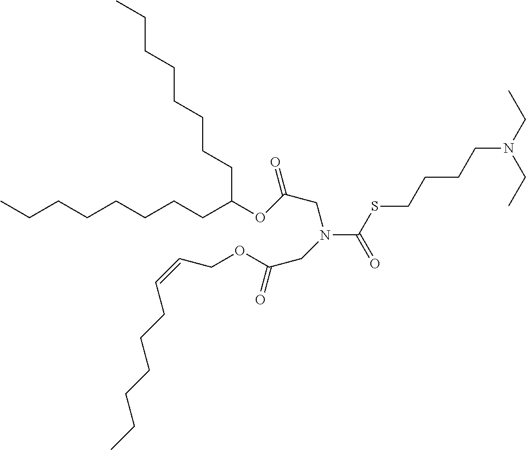 [SMILES: CCCCCC/C=C\COC(=O)CN(CC(=O)OC(CCCCCCCC)CCCCCCCC)C(=O)SCCCCN(CC)CC] | 0115 |
| 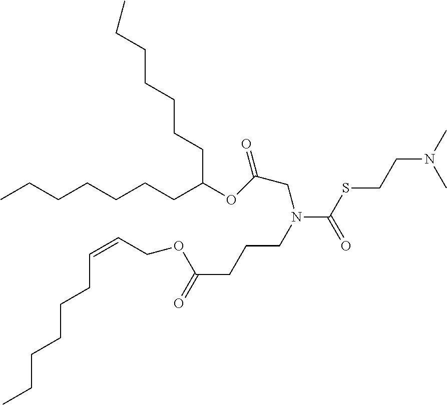 [SMILES: CCCCCC/C=C\COC(=O)CCCN(CC(=O)OC(CCCCCCC)CCCCCCC)C(=O)SCCN(C)C] | 0101 |
| 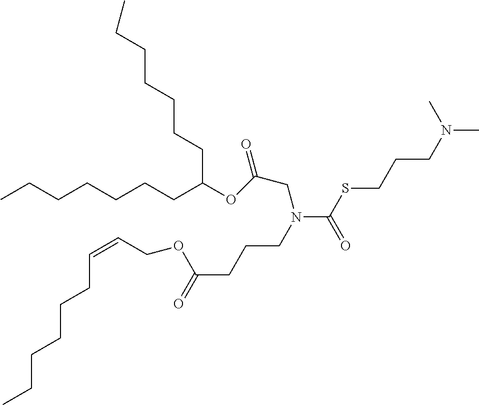 [SMILES: CCCCCC/C=C\COC(=O)CCCN(CC(=O)OC(CCCCCCC)CCCCCCC)C(=O)SCCCN(C)C] | 0106 |
| 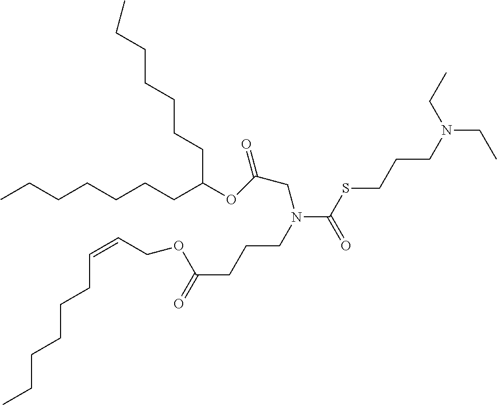 [SMILES: CCCCCC/C=C\COC(=O)CCCN(CC(=O)OC(CCCCCCC)CCCCCCC)C(=O)SCCCN(CC)CC] | 0116 |
| 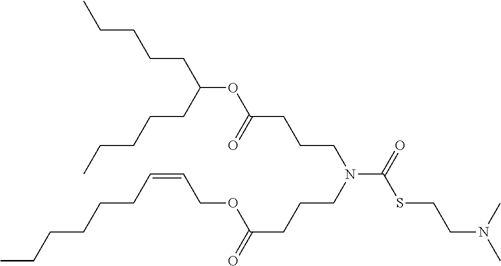 [SMILES: CCCCCC/C=C\COC(=O)CCCN(CCCC(=O)OC(CCCCC)CCCCC)C(=O)SCCN(C)C] | 0043 |
| 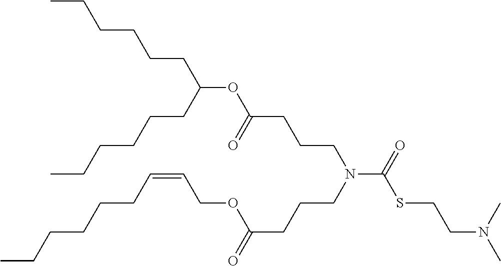 [SMILES: CCCCCC/C=C\COC(=O)CCCN(CCCC(=O)OC(CCCCCC)CCCCCC)C(=O)SCCN(C)C] | 0086 |
| 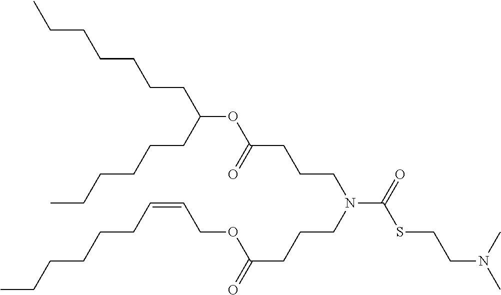 [SMILES: CCCCCC/C=C\COC(=O)CCCN(CCCC(=O)OC(CCCCCC)CCCCCCC)C(=O)SCCN(C)C] | 0058 |
| 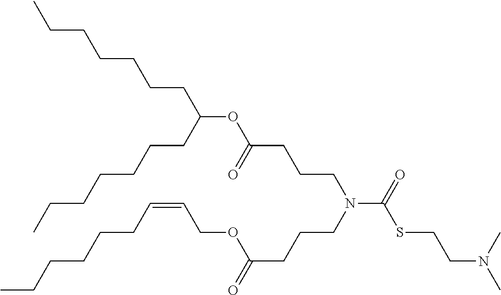 [SMILES: CCCCCC/C=C\COC(=O)CCCN(CCCC(=O)OC(CCCCCCC)CCCCCCC)C(=O)SCCN(C)C] | 0081 |
| 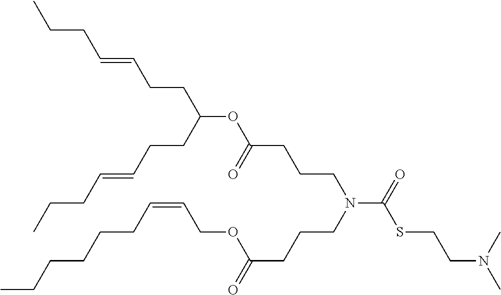 [SMILES: CCC/C=C/CCC(CC/C=C/CCC)OC(=O)CCCN(CCCC(=O)OC/C=C\CCCCCC)C(=O)SCCN(C)C] | 0123 |
| 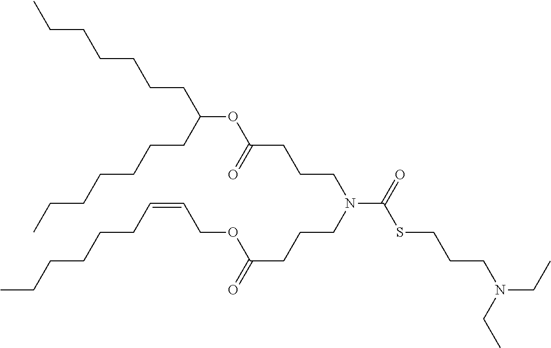 [SMILES: CCCCCC/C=C\COC(=O)CCCN(CCCC(=O)OC(CCCCCCC)CCCCCCC)C(=O)SCCCN(CC)CC] | 0122 |
| 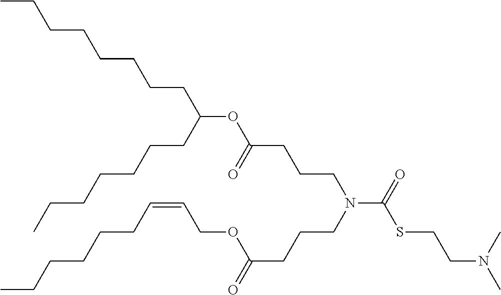 [SMILES: CCCCCC/C=C\COC(=O)CCCN(CCCC(=O)OC(CCCCCCC)CCCCCCCC)C(=O)SCCN(C)C] | 0057 |
| 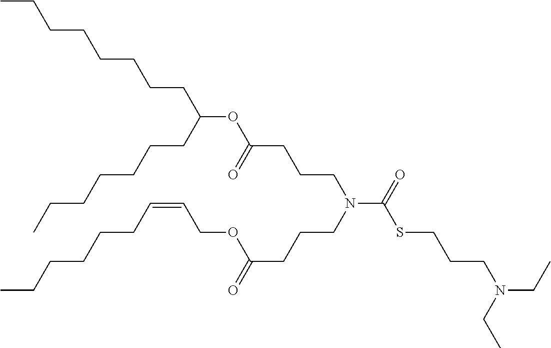 [SMILES: CCCCCC/C=C\COC(=O)CCCN(CCCC(=O)OC(CCCCCCC)CCCCCCCC)C(=O)SCCCN(CC)CC] | 0088 |
| 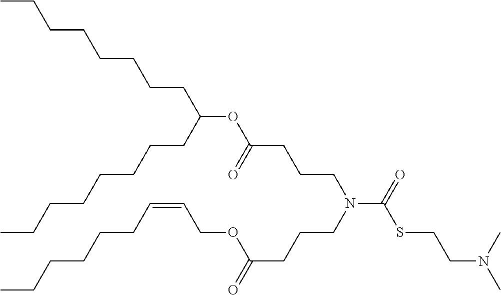 [SMILES: CCCCCC/C=C\COC(=O)CCCN(CCCC(=O)OC(CCCCCCCC)CCCCCCCC)C(=O)SCCN(C)C] | 0087 |
| 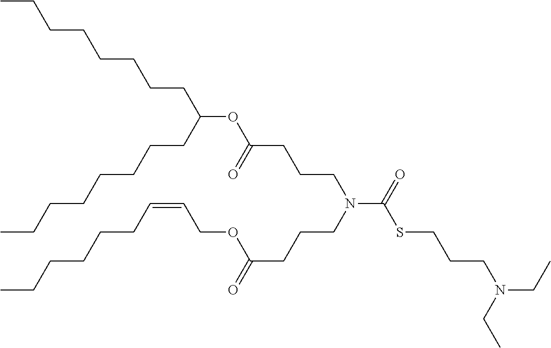 [SMILES: CCCCCC/C=C\COC(=O)CCCN(CCCC(=O)OC(CCCCCCCC)CCCCCCCC)C(=O)SCCCN(CC)CC] | 0124 |
| 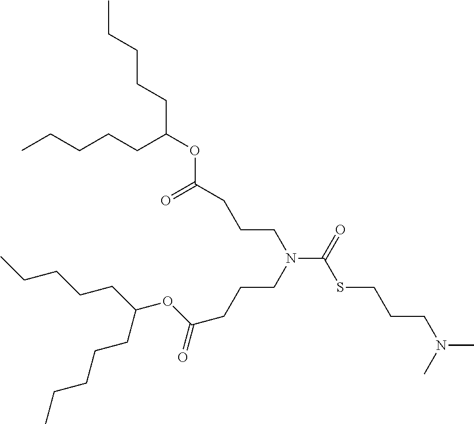 [SMILES: CCCCCC(CCCCC)OC(=O)CCCN(CCCC(=O)OC(CCCCC)CCCCC)C(=O)SCCCN(C)C] | 0128 |
| 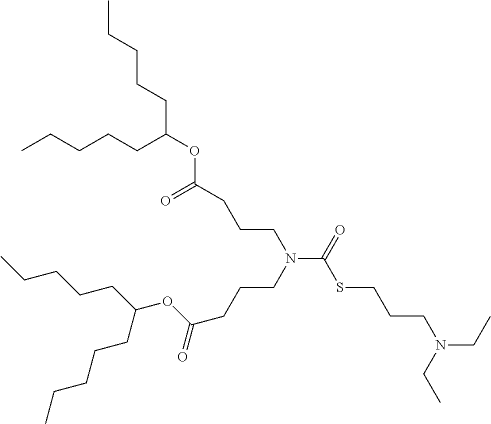 [SMILES: CCCCCC(CCCCC)OC(=O)CCCN(CCCC(=O)OC(CCCCC)CCCCC)C(=O)SCCCN(CC)CC] | 0127 |
| 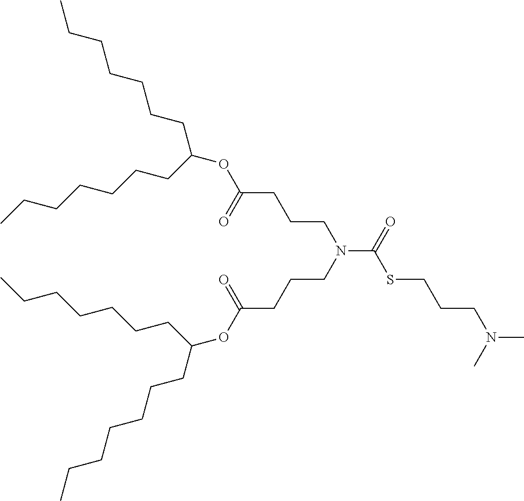 [SMILES: CCCCCCCC(CCCCCCC)OC(=O)CCCN(CCCC(=O)OC(CCCCCCC)CCCCCCC)C(=O)SCCCN(C)C] | 0126 |
| 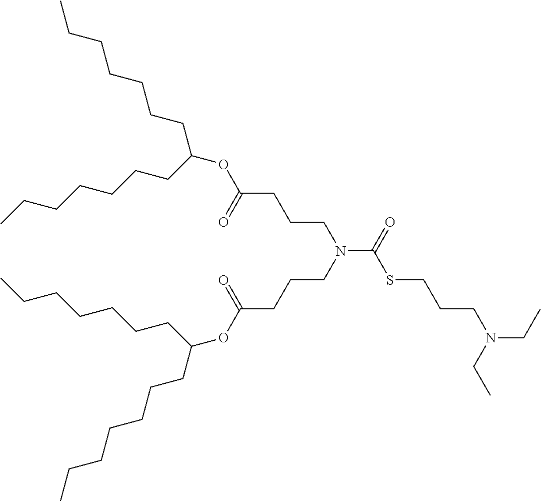 [SMILES: CCCCCCCC(CCCCCCC)OC(=O)CCCN(CCCC(=O)OC(CCCCCCC)CCCCCCC)C(=O)SCCCN(CC)CC] | 0129 |
| 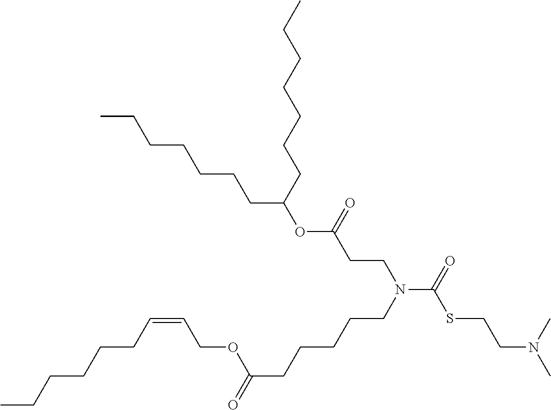 [SMILES: CCCCCC/C=C\COC(=O)CCCCCN(CCC(=O)OC(CCCCCCC)CCCCCCC)C(=O)SCCN(C)C] | 0082 |
| 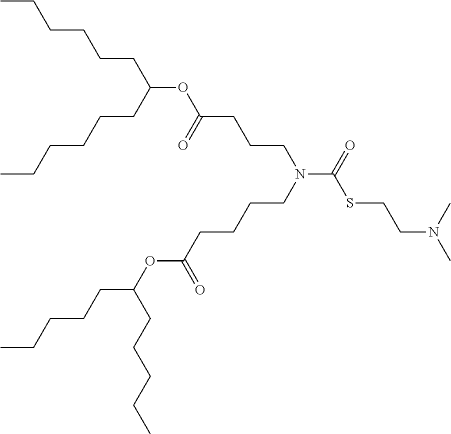 [SMILES: CCCCCCC(CCCCCC)OC(=O)CCCN(CCCCC(=O)OC(CCCCC)CCCCC)C(=O)SCCN(C)C] | 0085 |
| 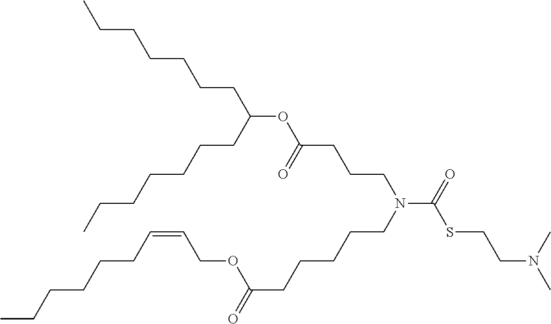 [SMILES: CCCCCC/C=C\COC(=O)CCCCCN(CCCC(=O)OC(CCCCCCC)CCCCCCC)C(=O)SCCN(C)C] | 0083 |
| 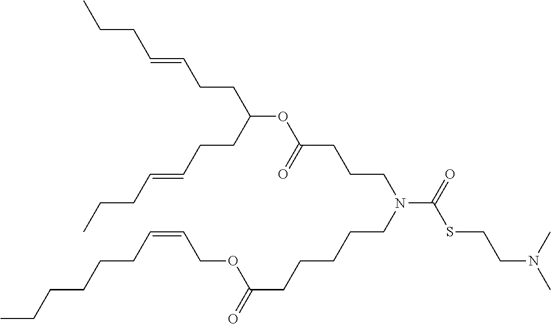 [SMILES: CCC/C=C/CCC(CC/C=C/CCC)OC(=O)CCCN(CCCCCC(=O)OC/C=C\CCCCCC)C(=O)SCCN(C)C] | 0121 |
| 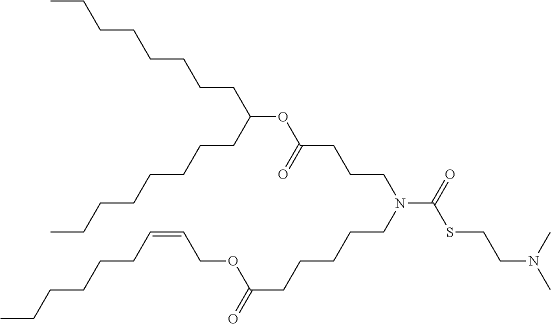 [SMILES: CCCCCC/C=C\COC(=O)CCCCCN(CCCC(=O)OC(CCCCCCCC)CCCCCCCC)C(=O)SCCN(C)C] | 0091 |
| 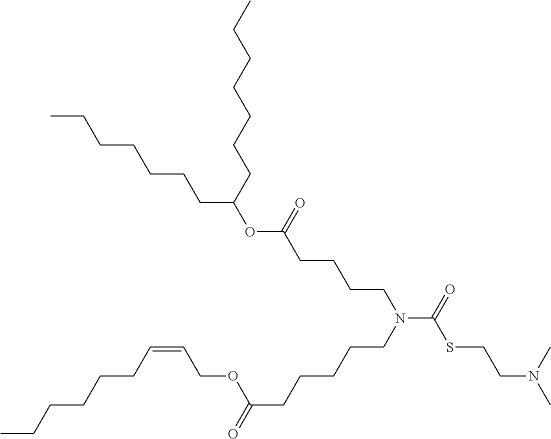 [SMILES: CCCCCC/C=C\COC(=O)CCCCCN(CCCCC(=O)OC(CCCCCCC)CCCCCCC)C(=O)SCCN(C)C] | 0102 |
| 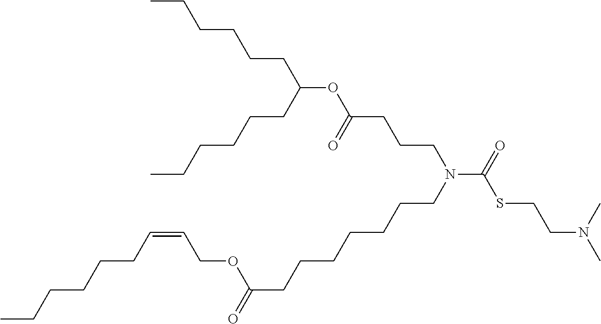 [SMILES: CCCCCC/C=C\COC(=O)CCCCCCCN(CCCC(=O)OC(CCCCCC)CCCCCC)C(=O)SCCN(C)C] | 0098 |
| 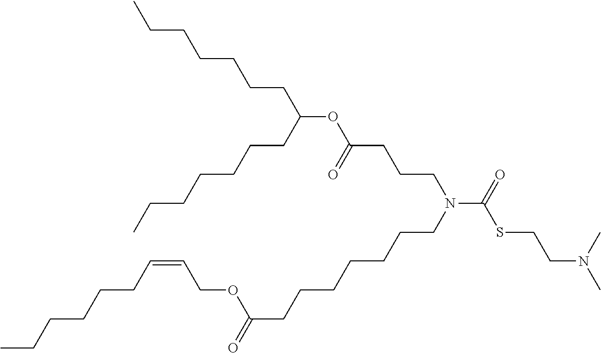 [SMILES: CCCCCC/C=C\COC(=O)CCCCCCCN(CCCC(=O)OC(CCCCCCC)CCCCCCC)C(=O)SCCN(C)C] | 0092 |
| 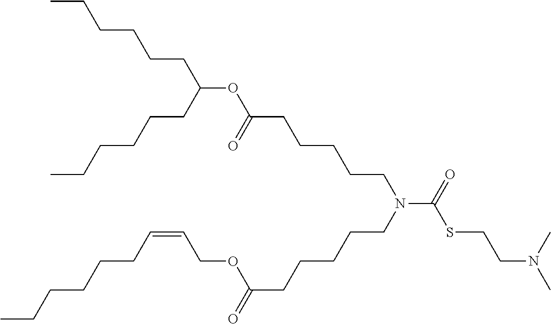 [SMILES: CCCCCC/C=C\COC(=O)CCCCCN(CCCCCC(=O)OC(CCCCCC)CCCCCC)C(=O)SCCN(C)C] | 0084 |
| 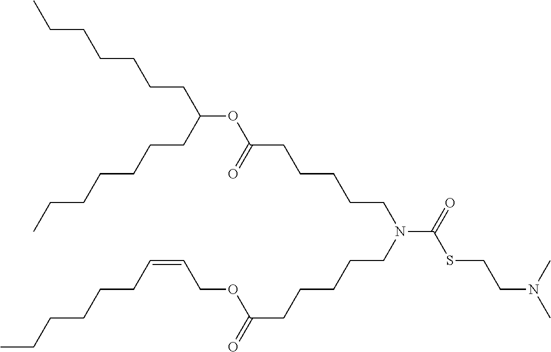 [SMILES: CCCCCC/C=C\COC(=O)CCCCCN(CCCCCC(=O)OC(CCCCCCC)CCCCCCC)C(=O)SCCN(C)C] | 0095 |
| 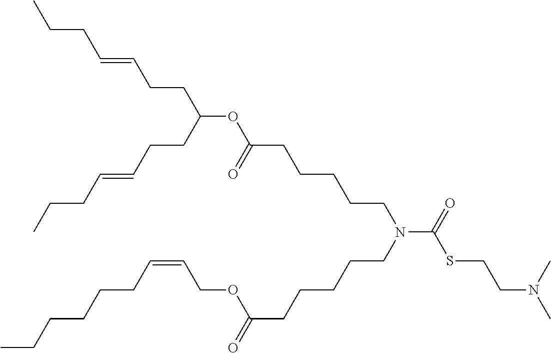 [SMILES: CCC/C=C/CCC(CC/C=C/CCC)OC(=O)CCCCCN(CCCCCC(=O)OC/C=C\CCCCCC)C(=O)SCCN(C)C] | 0125 |
| 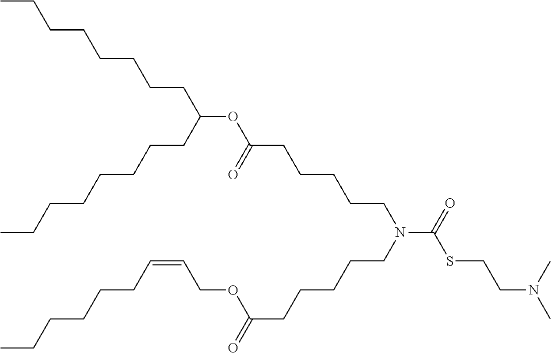 [SMILES: CCCCCC/C=C\COC(=O)CCCCCN(CCCCCC(=O)OC(CCCCCCCC)CCCCCCCC)C(=O)SCCN(C)C] | 0094 |
| 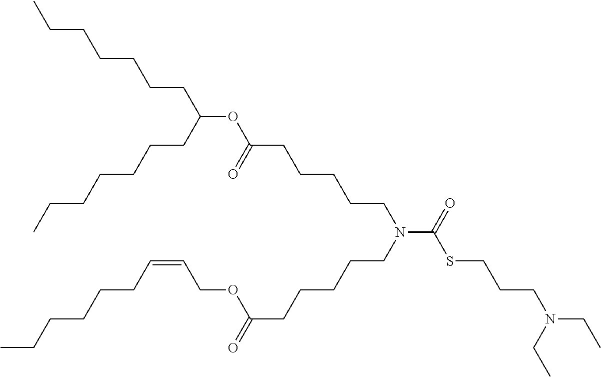 [SMILES: CCCCCC/C=C\COC(=O)CCCCCN(CCCCCC(=O)OC(CCCCCCC)CCCCCCC)C(=O)SCCCN(CC)CC] | 0109 |
| 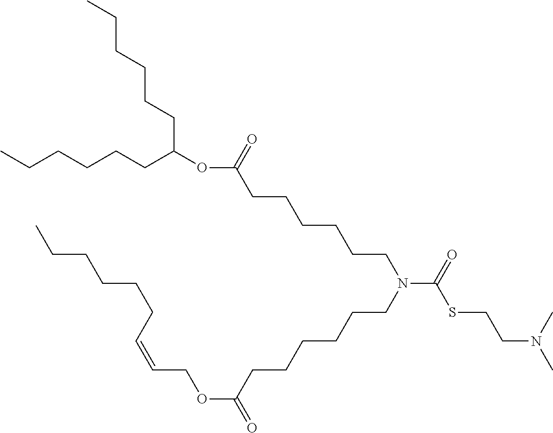 [SMILES: CCCCCC/C=C\COC(=O)CCCCCCN(CCCCCCC(=O)OC(CCCCCC)CCCCCC)C(=O)SCCN(C)C] | 0110 |
| 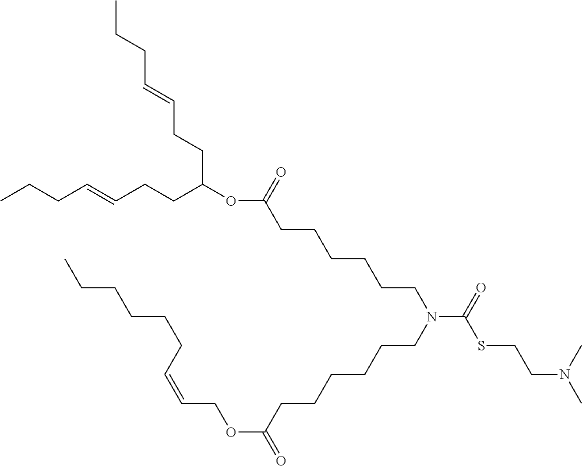 [SMILES: CCC/C=C/CCC(CC/C=C/CCC)OC(=O)CCCCCCN(CCCCCCC(=O)OC/C=C\CCCCCC)C(=O)SCCN(C)C] | 0118 |
| 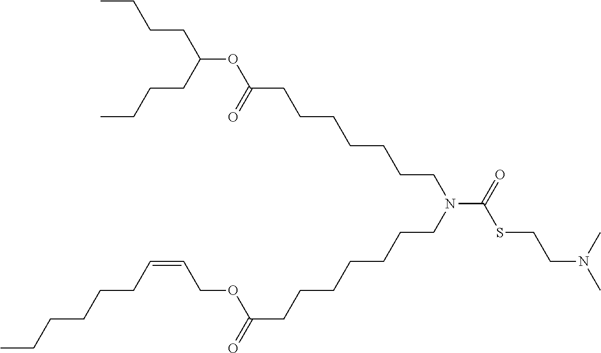 [SMILES: CCCCCC/C=C\COC(=O)CCCCCCCN(CCCCCCCC(=O)OC(CCCC)CCCC)C(=O)SCCN(C)C] | 0108 |
| 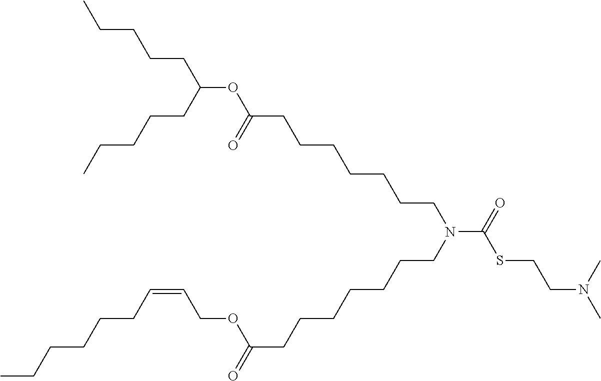 [SMILES: CCCCCC/C=C\COC(=O)CCCCCCCN(CCCCCCCC(=O)OC(CCCCC)CCCCC)C(=O)SCCN(C)C] | 0107 |
| 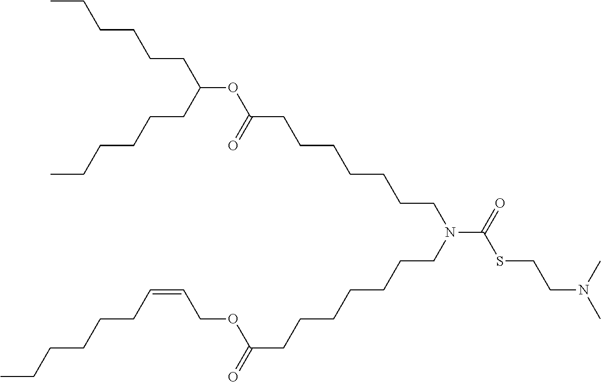 [SMILES: CCCCCC/C=C\COC(=O)CCCCCCCN(CCCCCCCC(=O)OC(CCCCCC)CCCCCC)C(=O)SCCN(C)C] | 0093 |
| 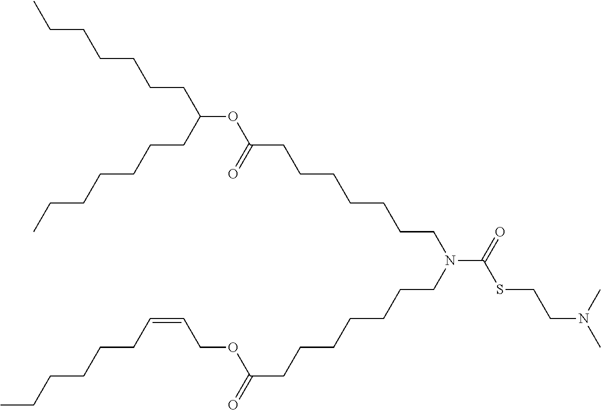 [SMILES: CCCCCC/C=C\COC(=O)CCCCCCCN(CCCCCCCC(=O)OC(CCCCCCC)CCCCCCC)C(=O)SCCN(C)C] | 0097 |
| 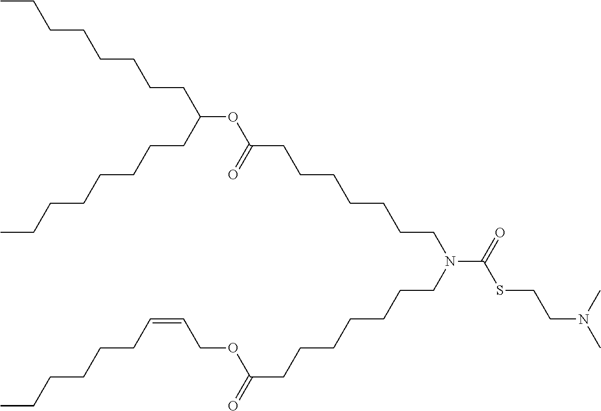 [SMILES: CCCCCC/C=C\COC(=O)CCCCCCCN(CCCCCCCC(=O)OC(CCCCCCCC)CCCCCCCC)C(=O)SCCN(C)C] | 0096 |

TABLE 15

| | |
|---|---|
| 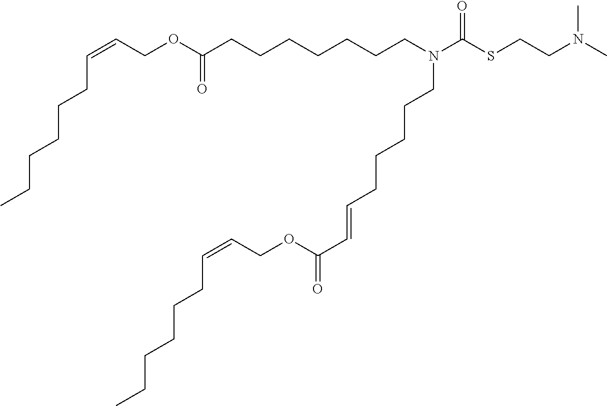 [SMILES: CCCCCC/C=C\COC(=O)/C=C/CCCCCN(CCCCCCCC(=O)OC/C=C\CCCCCC)C(=O)SCCN(C)C] | 11 |
| 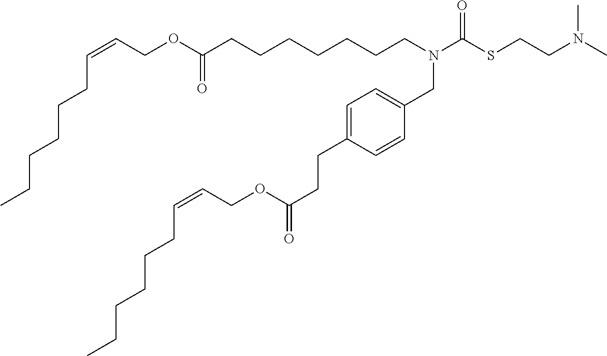 [SMILES: CCCCCC/C=C\COC(=O)CCCCCCCN(Cc1ccc(CCC(=O)OC/C=C\CCCCCC)cc1)C(=O)SCCN(C)C] | 13 |
| 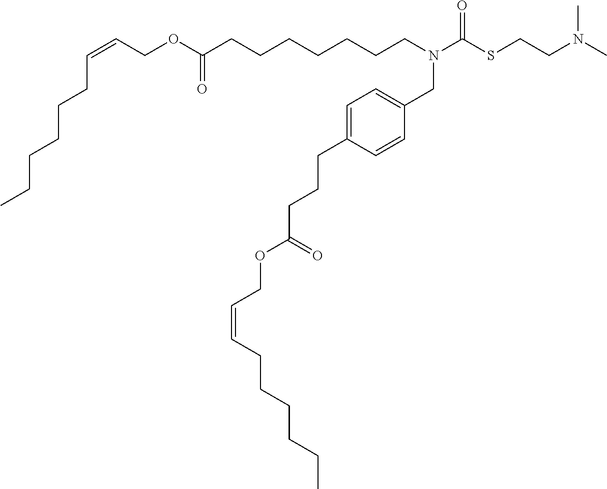 [SMILES: CCCCCC/C=C\COC(=O)CCCCCCCN(Cc1ccc(CCCC(=O)OC/C=C\CCCCCC)cc1)C(=O)SCCN(C)C] | 14 |
| 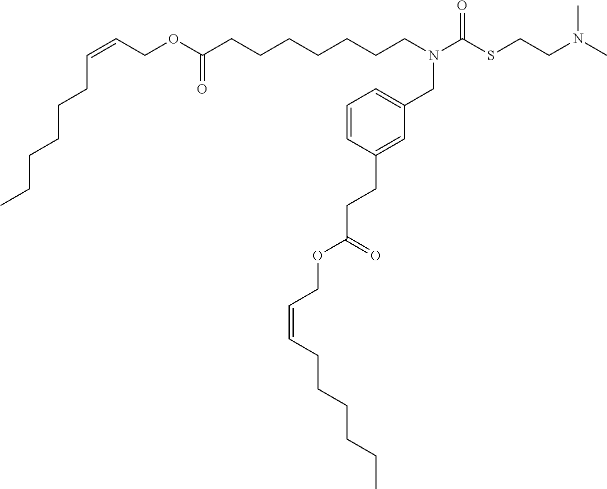 [SMILES: CCCCCC/C=C\COC(=O)CCCCCCCN(Cc1cccc(CCC(=O)OC/C=C\CCCCCC)c1)C(=O)SCCN(C)C] | 15 |
| 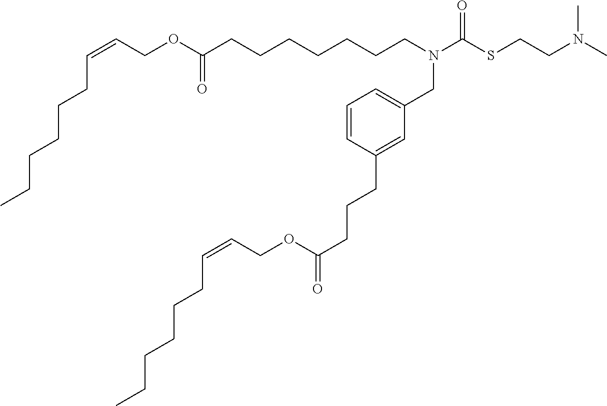 [SMILES: CCCCCC/C=C\COC(=O)CCCCCCCN(Cc1cccc(CCCC(=O)OC/C=C\CCCCCC)c1)C(=O)SCCN(C)C] | 16 |
| 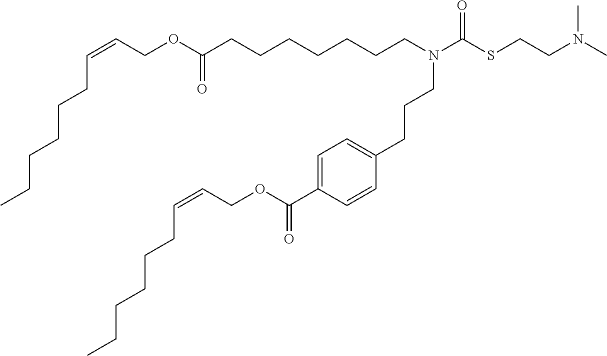 [SMILES: CCCCCC/C=C\COC(=O)CCCCCCCN(CCCc1ccc(C(=O)OC/C=C\CCCCCC)cc1)C(=O)SCCN(C)C] | 17 |
| 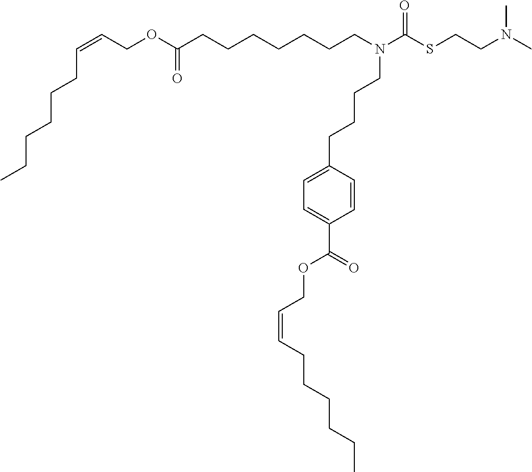 [SMILES: CCCCCC/C=C\COC(=O)CCCCCCCN(CCCCc1ccc(C(=O)OC/C=C\CCCCCC)cc1)C(=O)SCCN(C)C] | 18 |
| 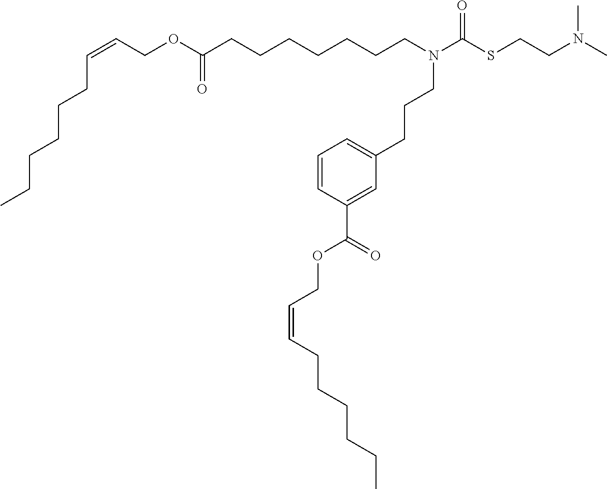 [SMILES: CCCCCC/C=C\COC(=O)CCCCCCCN(CCCc1cccc(C(=O)OC/C=C\CCCCCC)c1)C(=O)SCCN(C)C] | 19 |
| 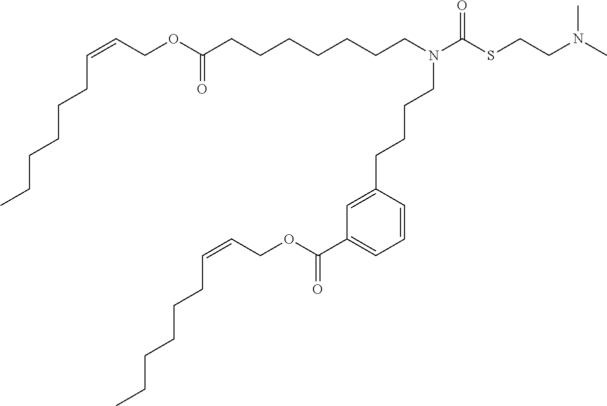 [SMILES: CCCCCC/C=C\COC(=O)CCCCCCCN(CCCCc1cccc(C(=O)OC/C=C\CCCCCC)c1)C(=O)SCCN(C)C] | 20 |

In some embodiments, an ionizable lipid is as described in international patent application PCT/US2019/015913. In some embodiments, an ionizable lipid is chosen from the following:

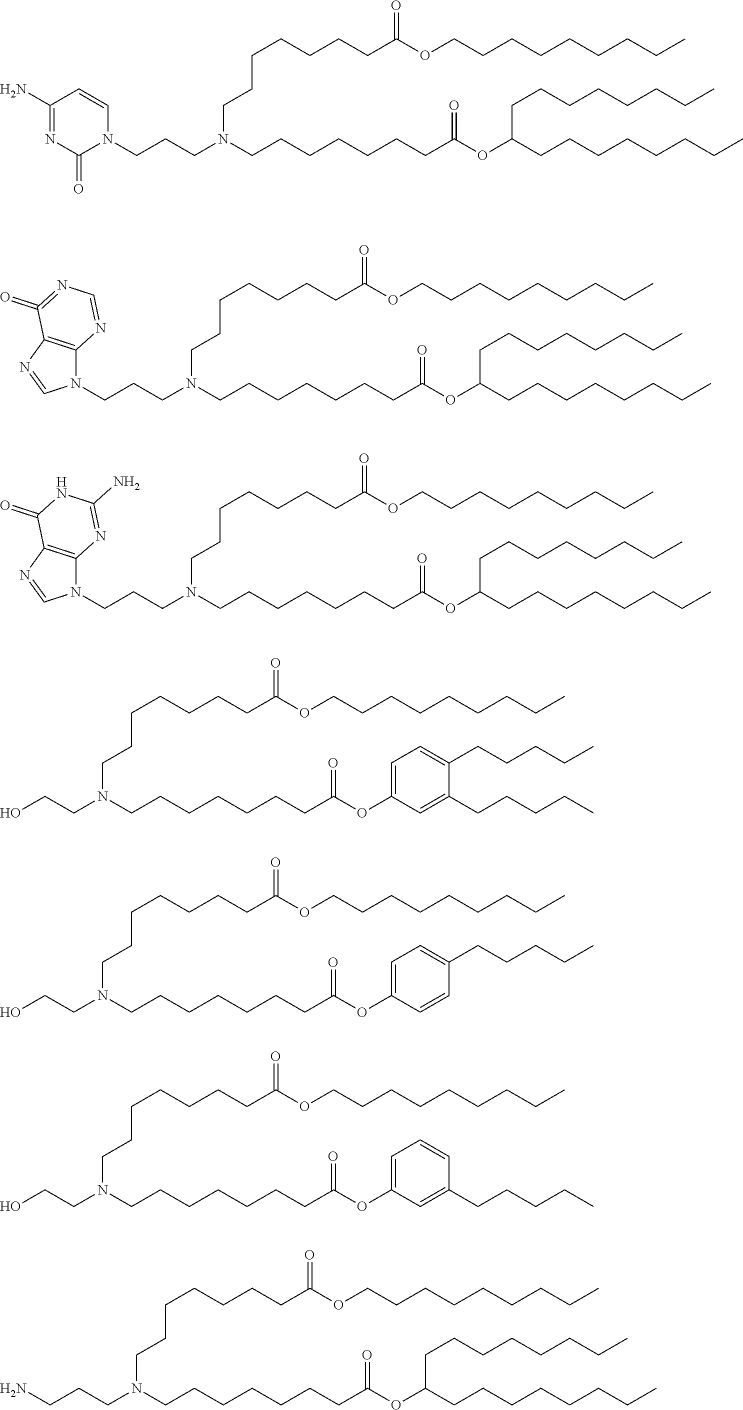

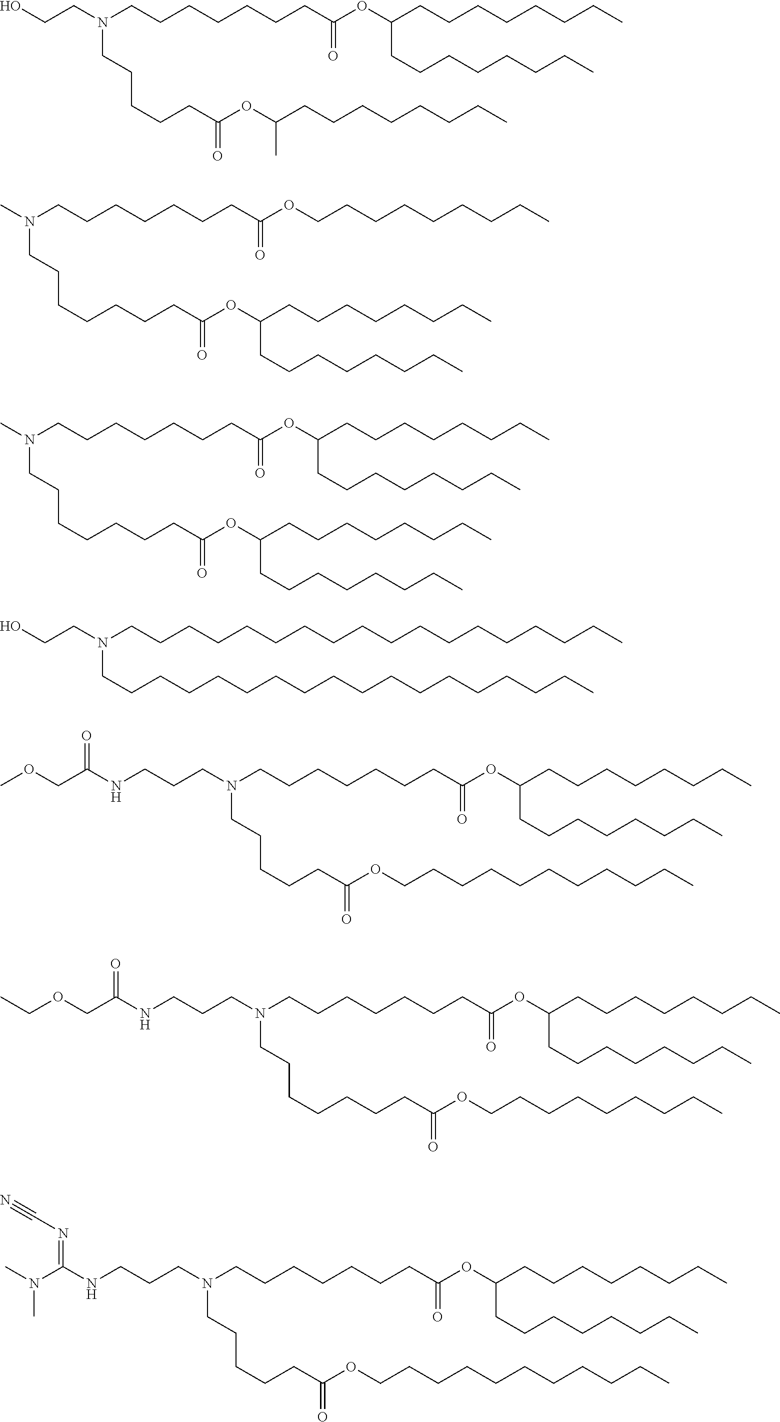

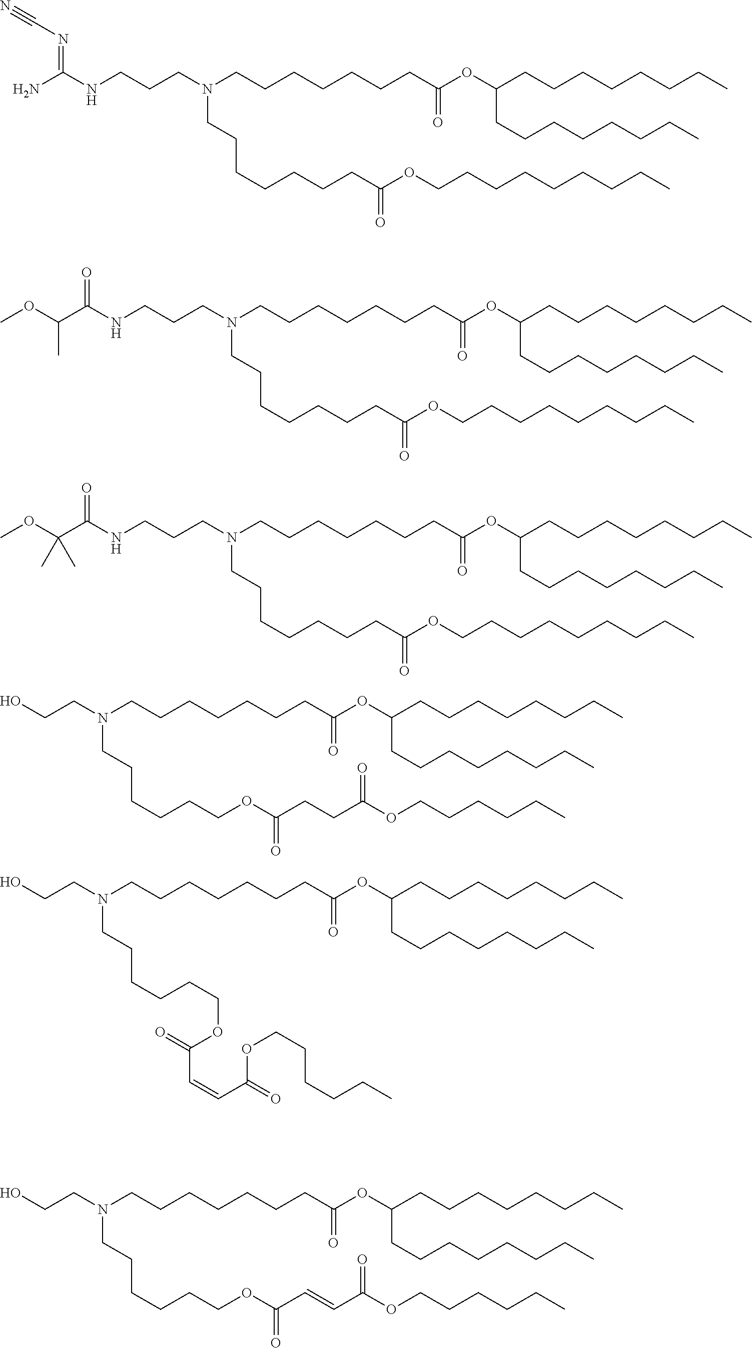

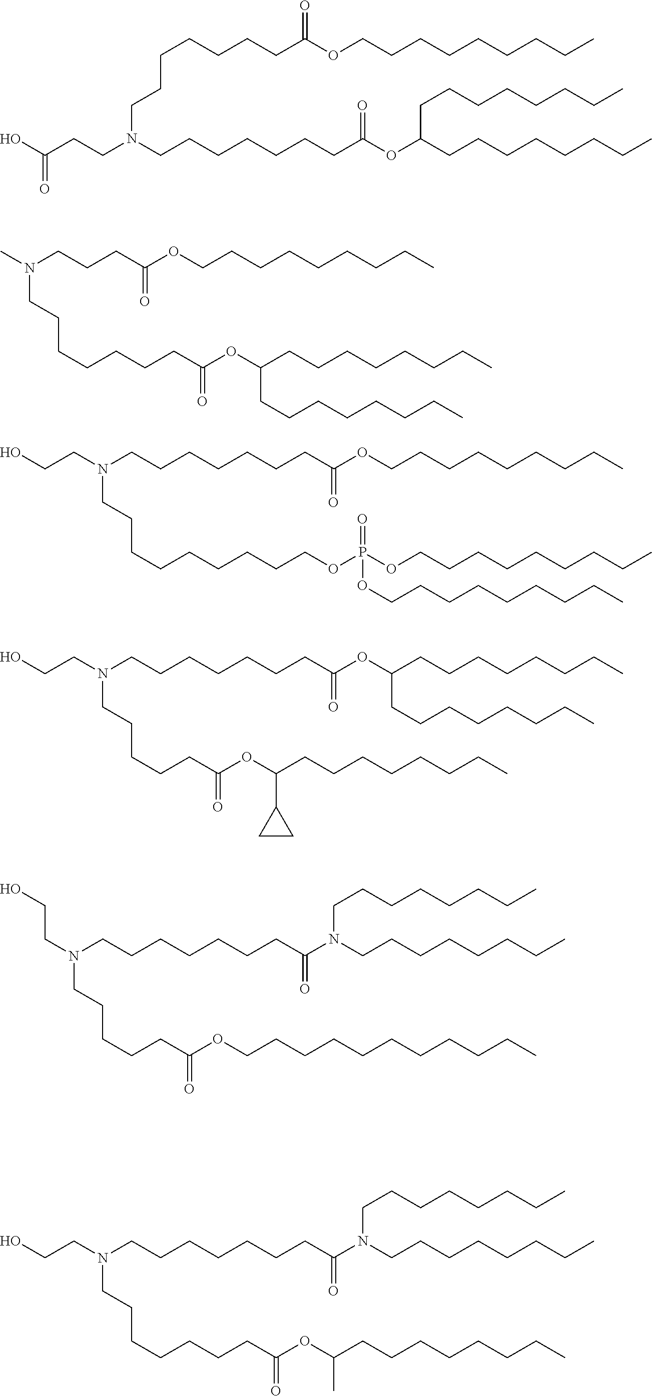

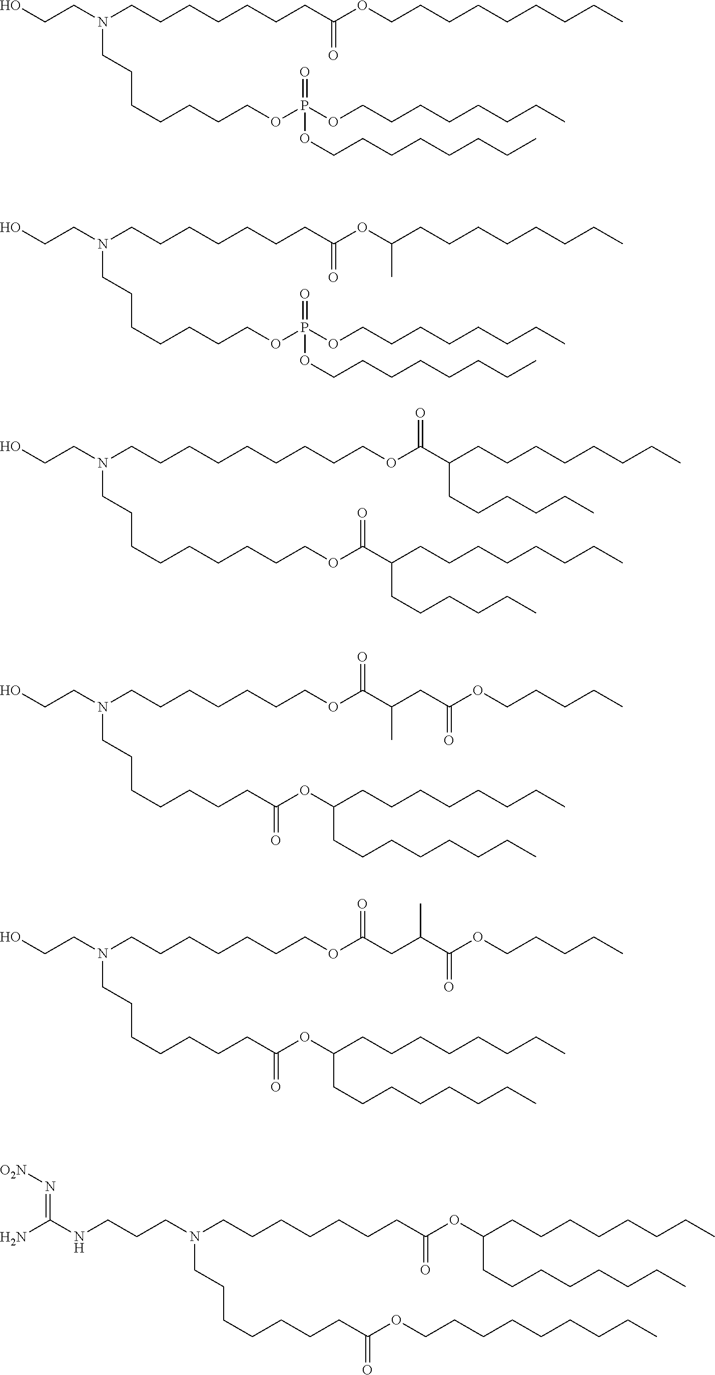

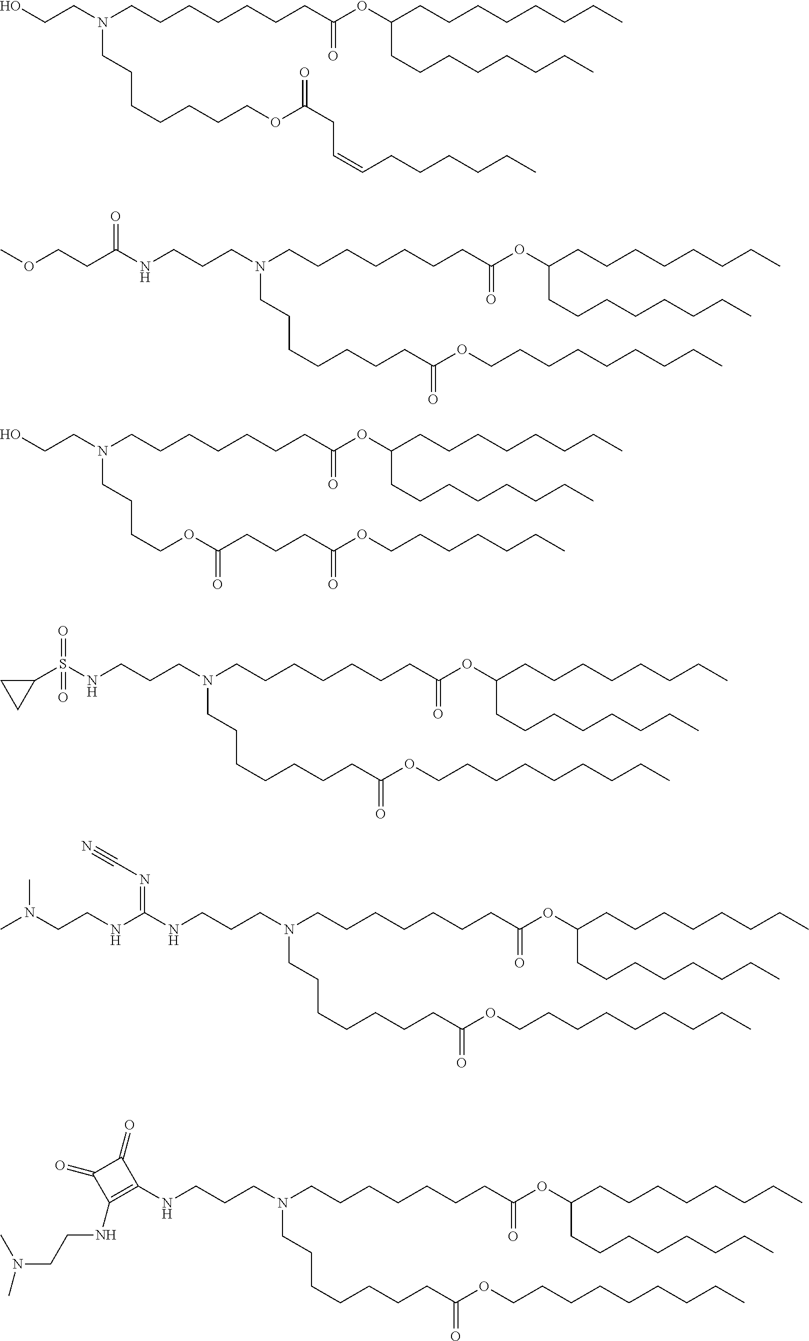

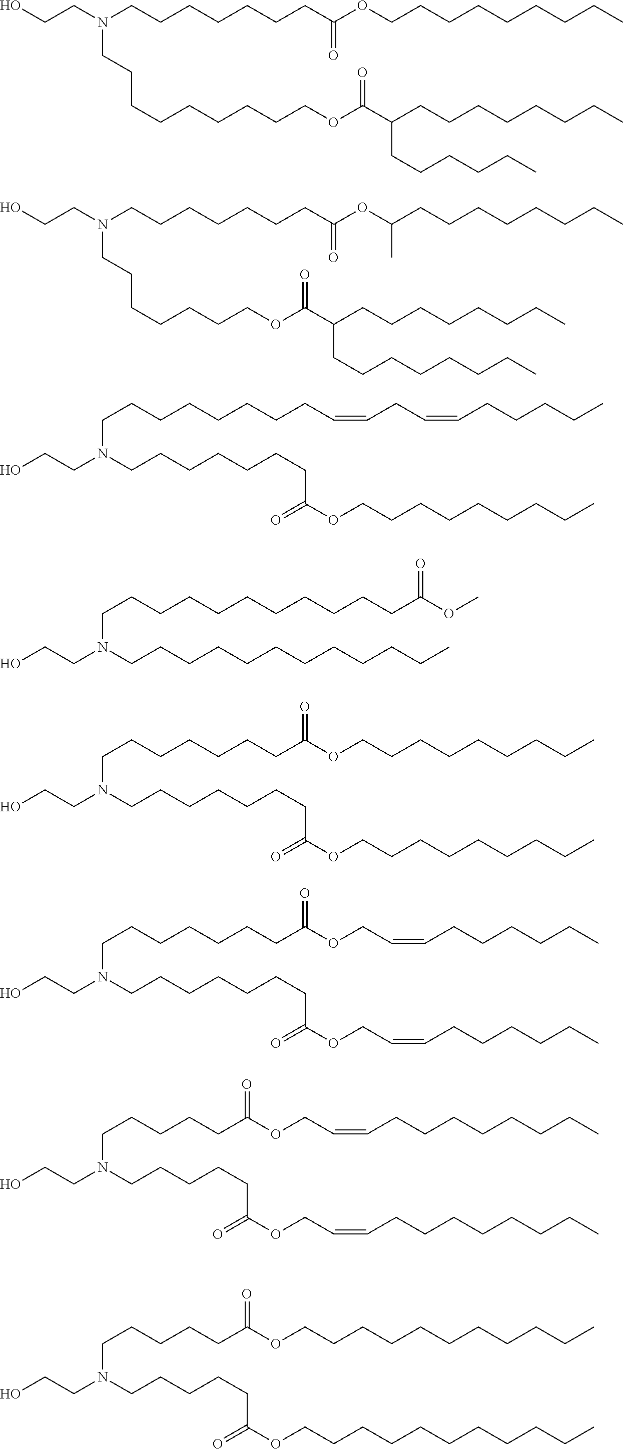

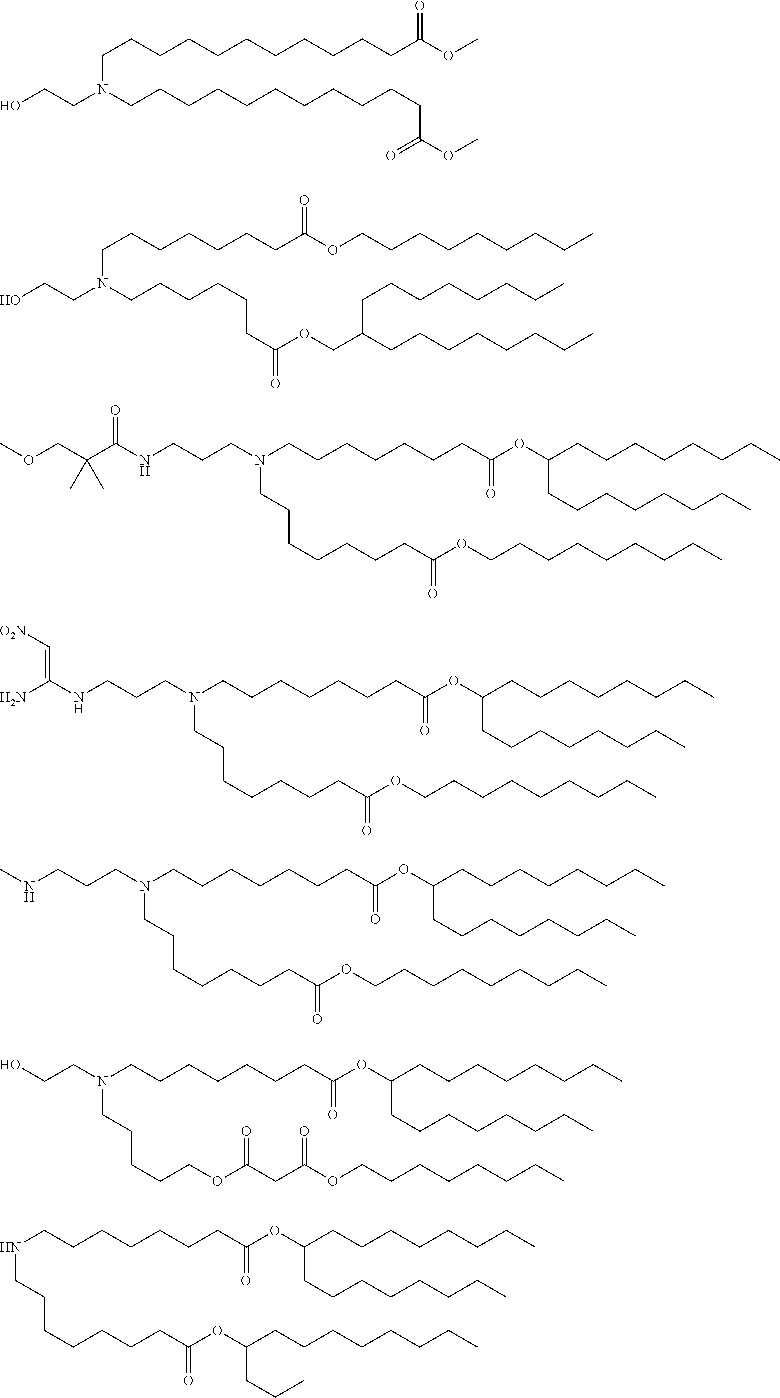

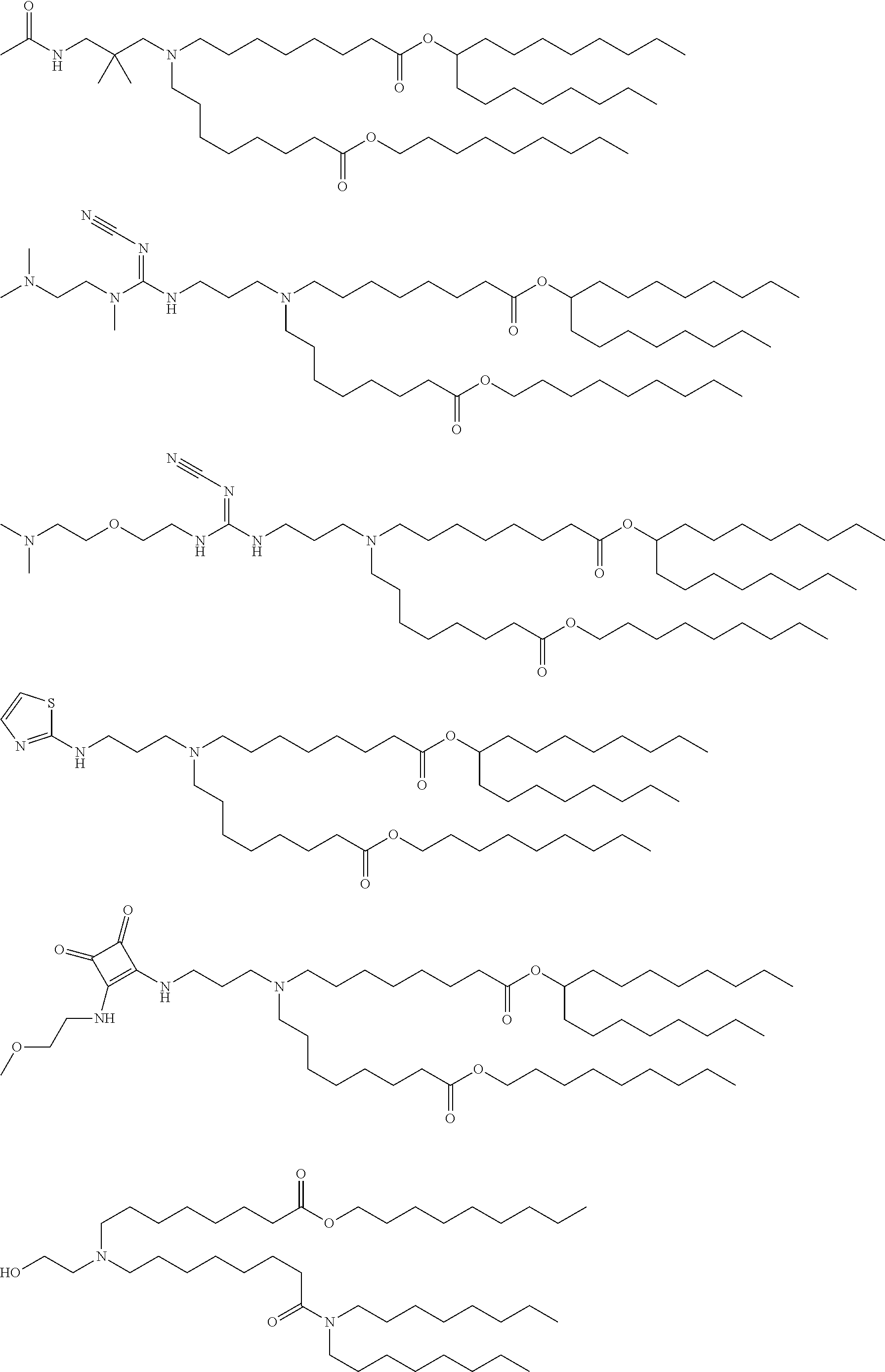

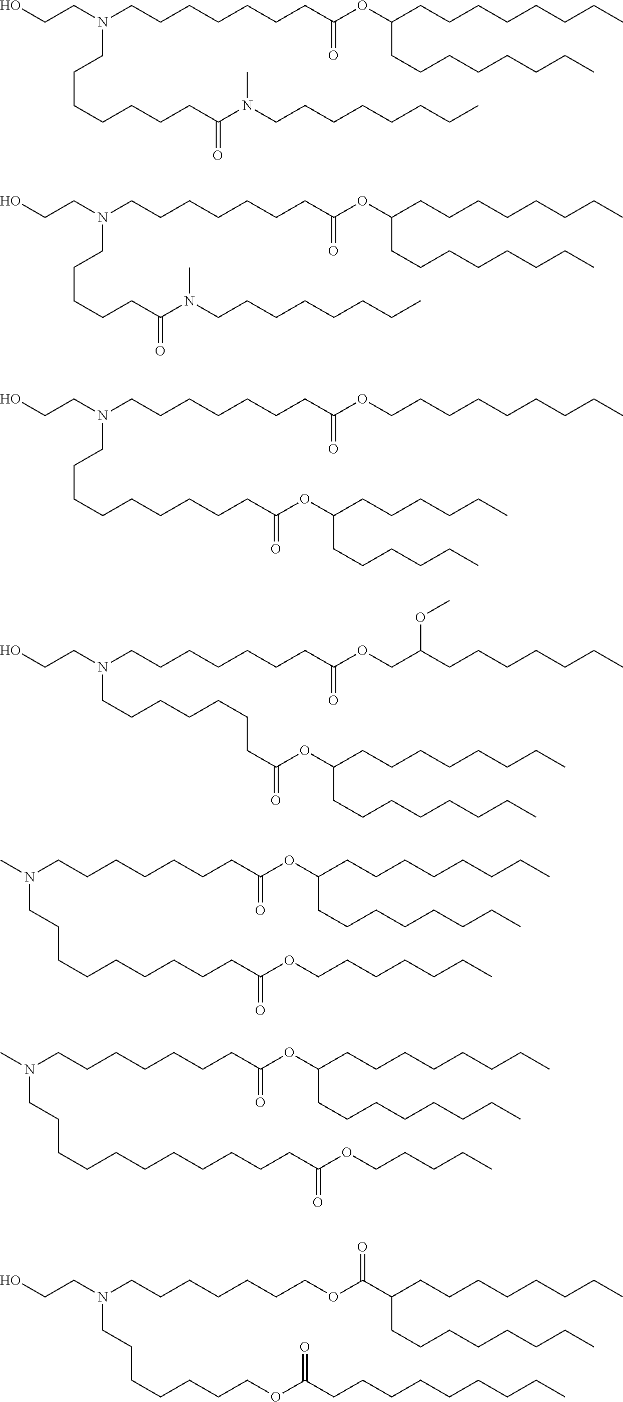

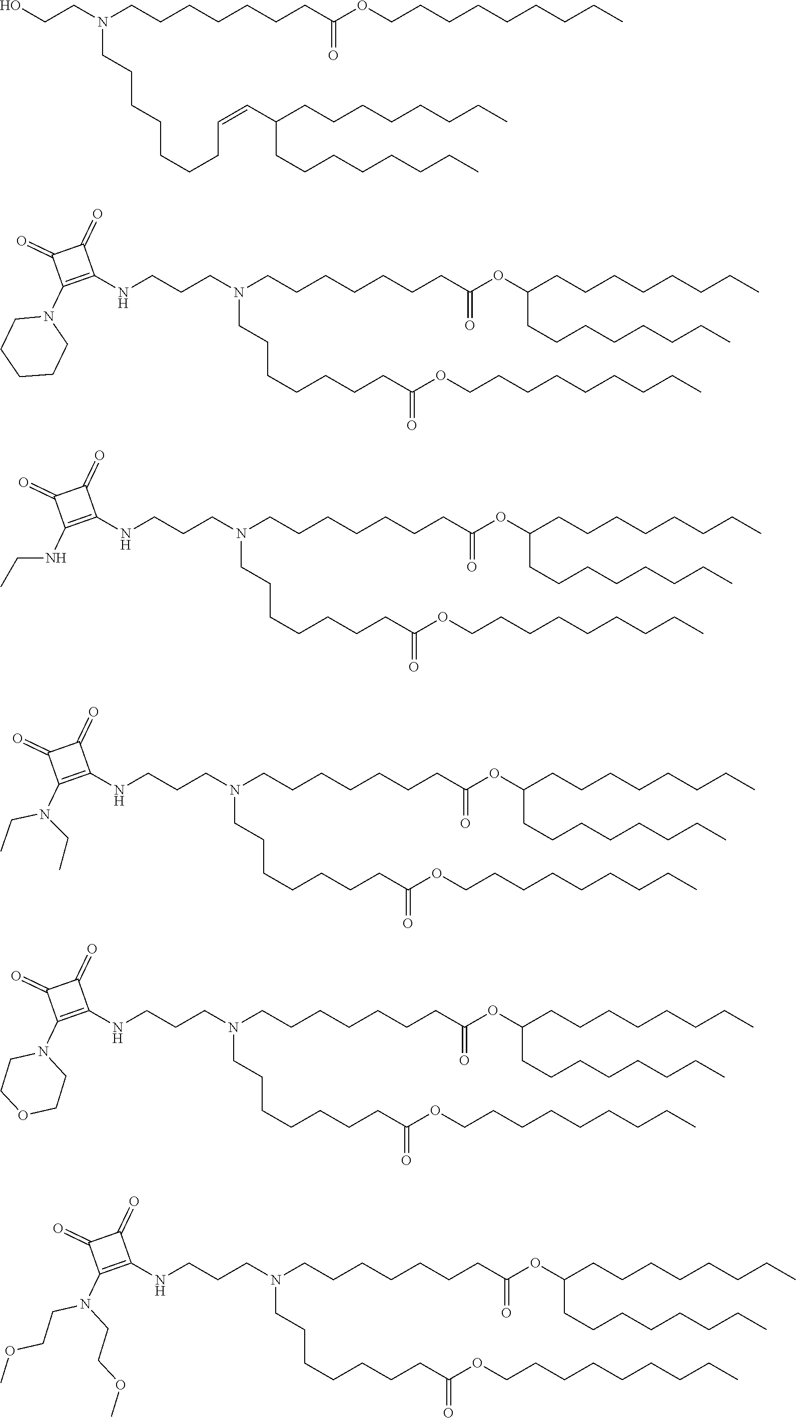

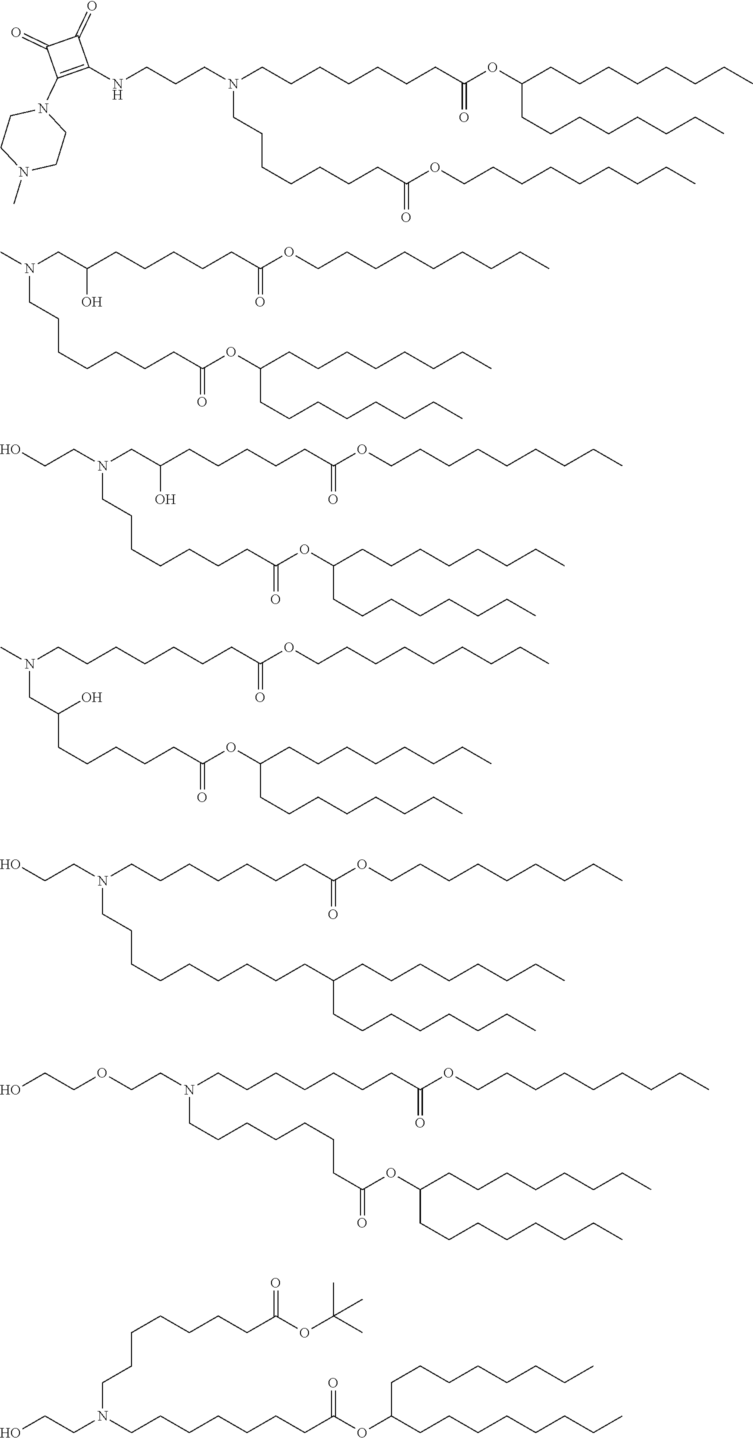

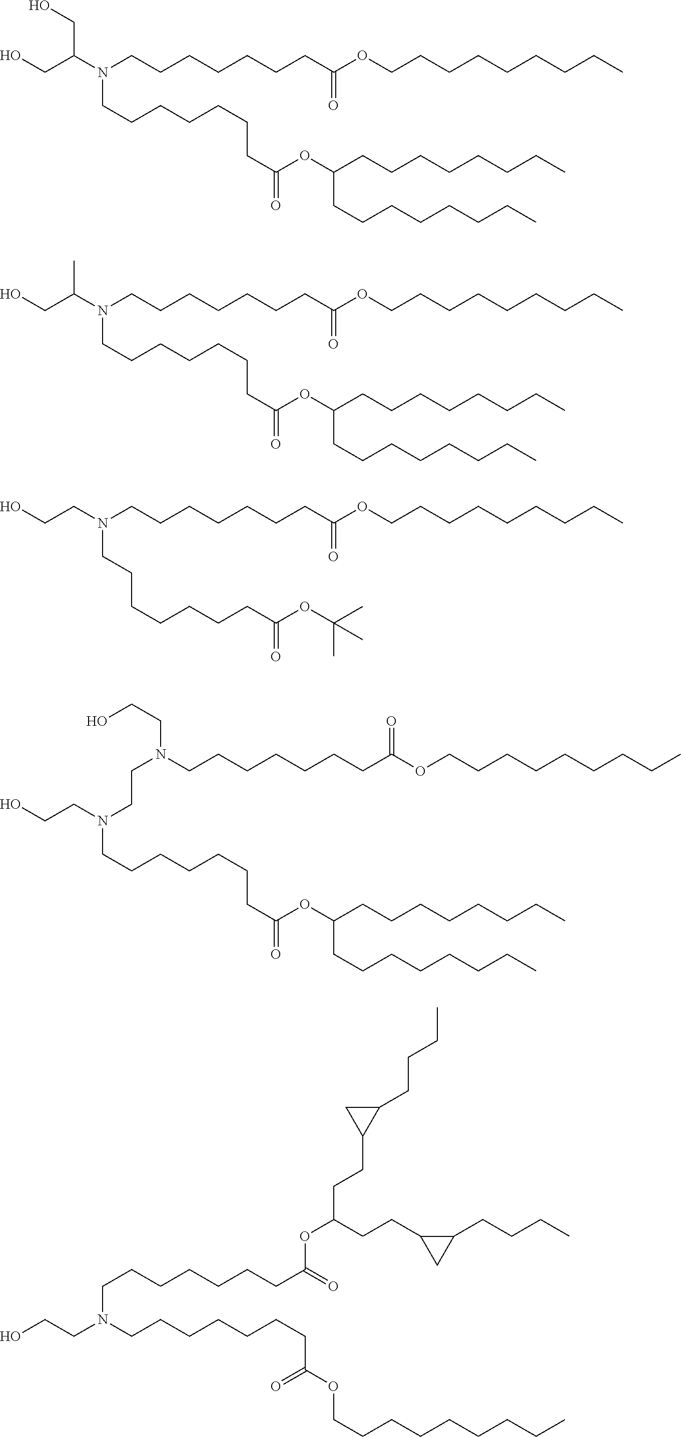

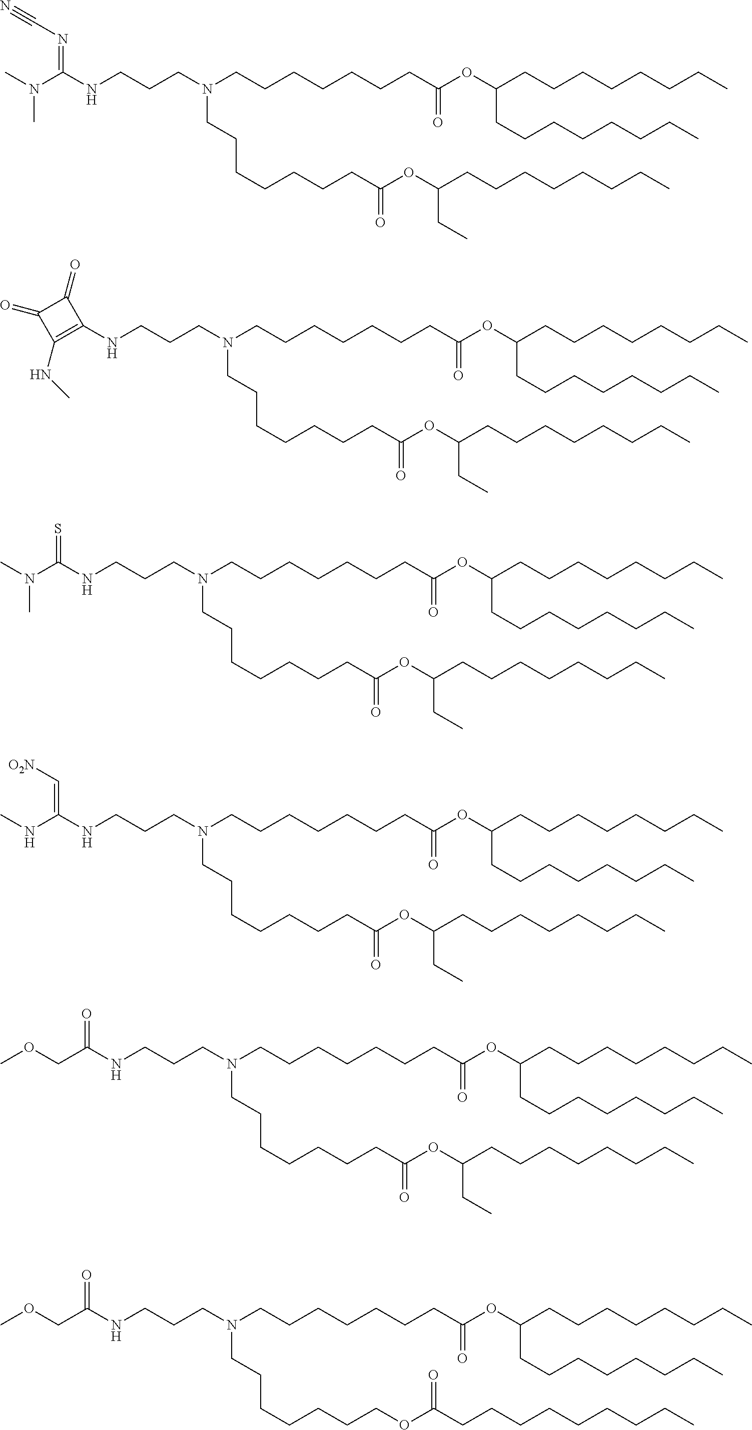

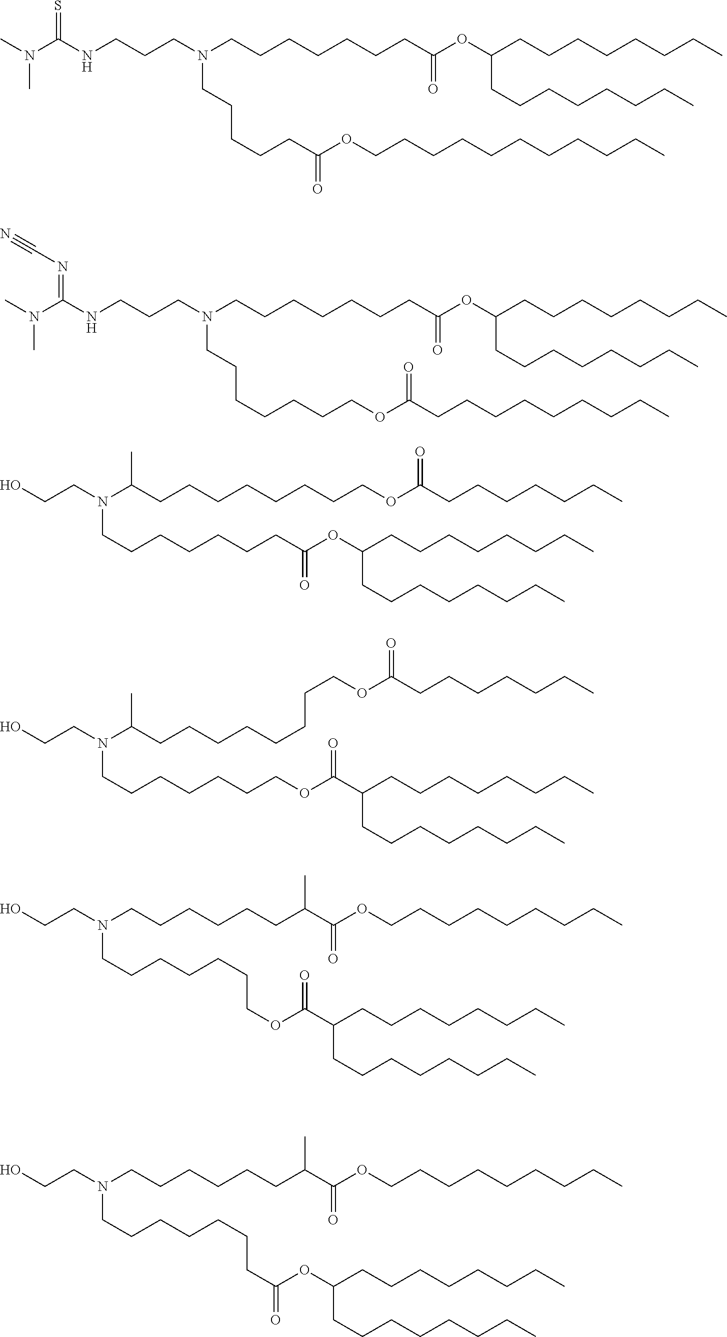

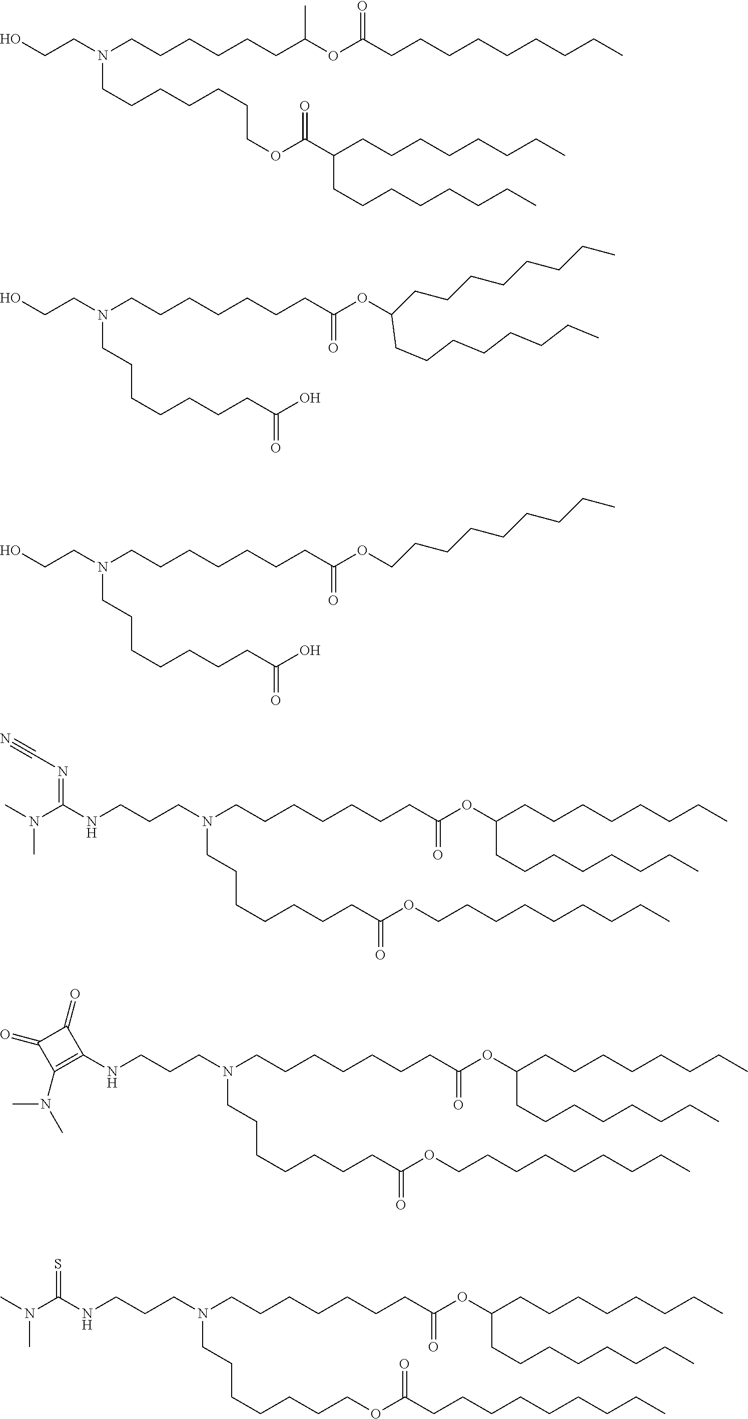

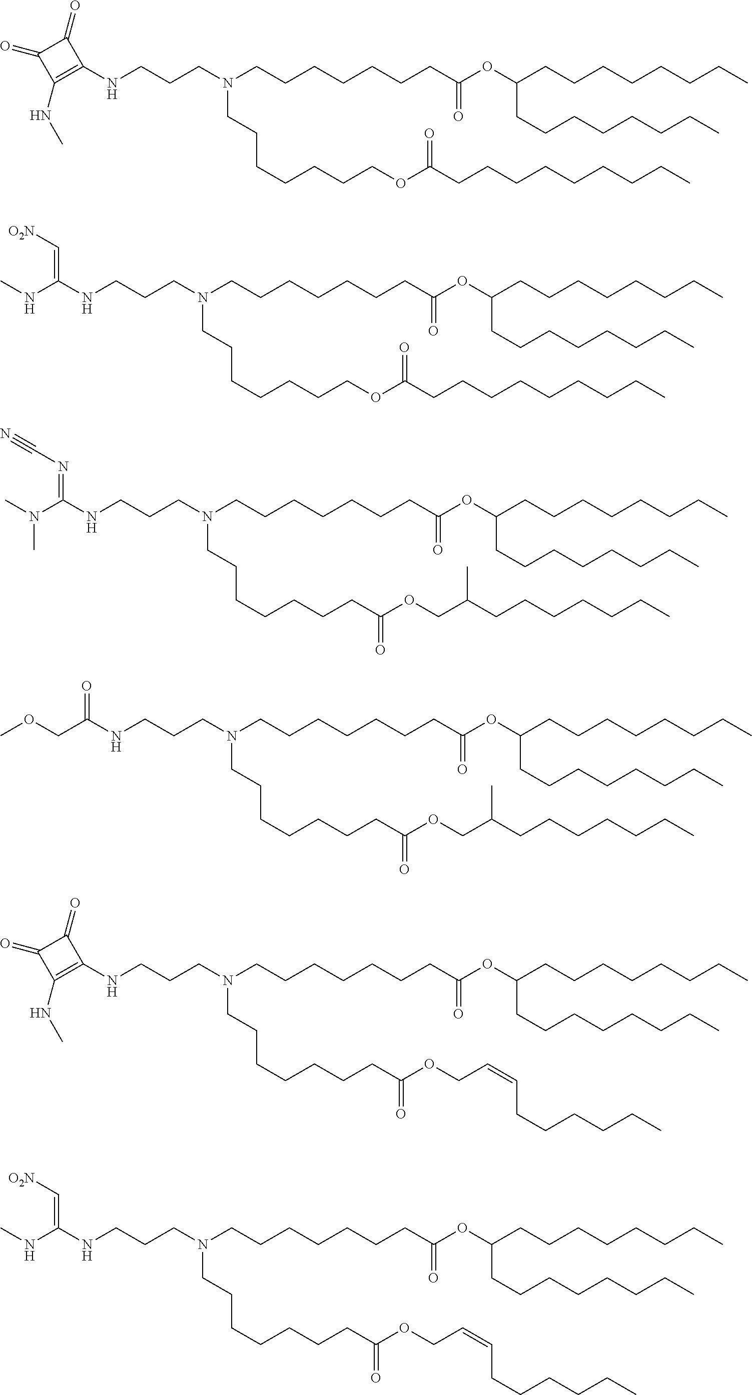

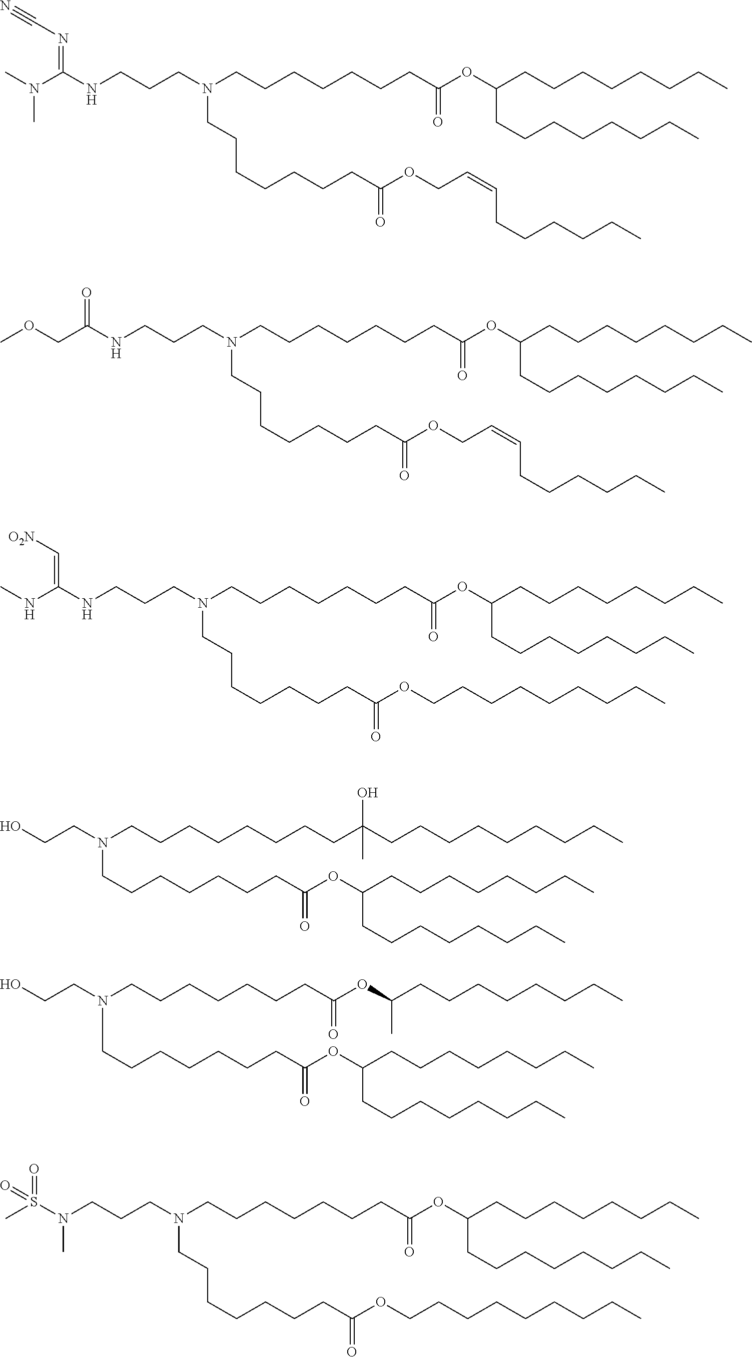

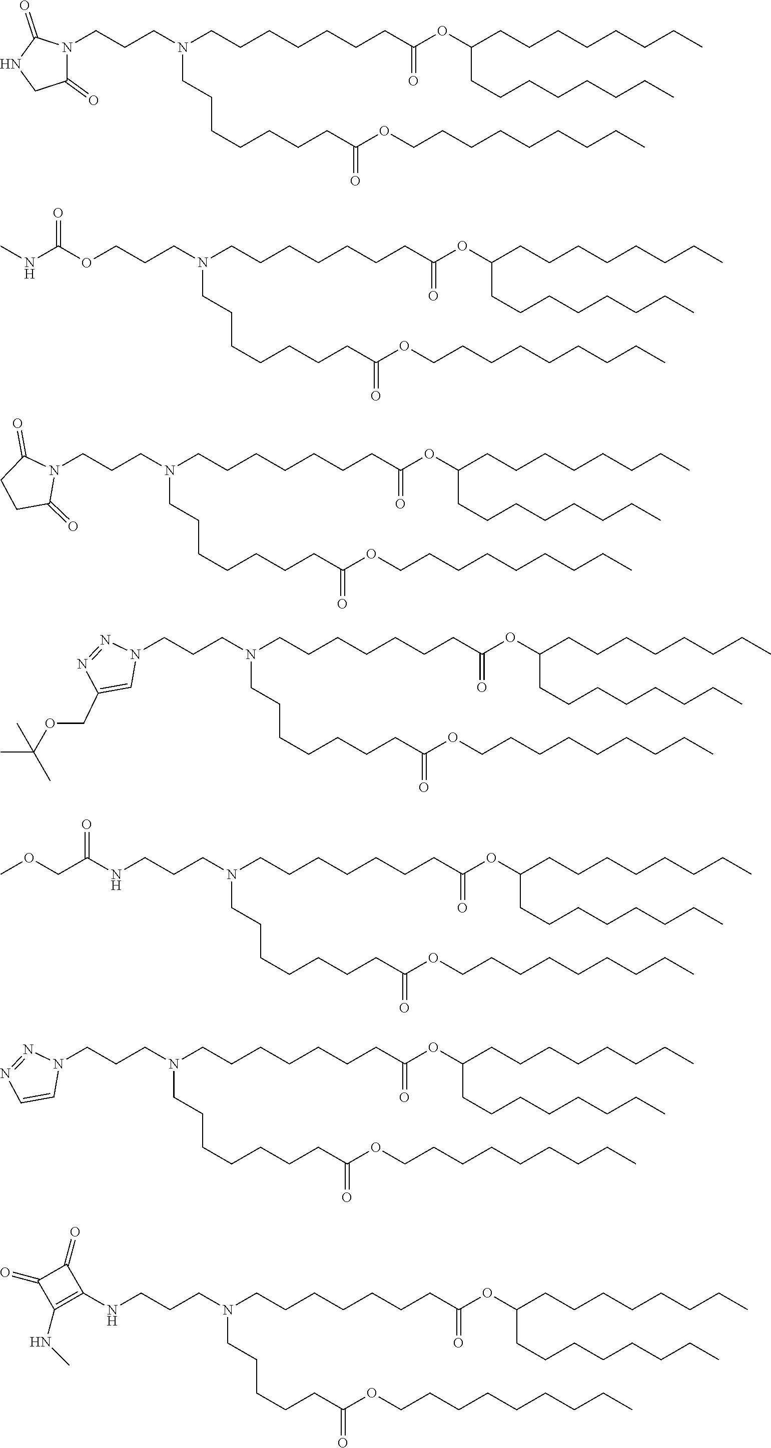

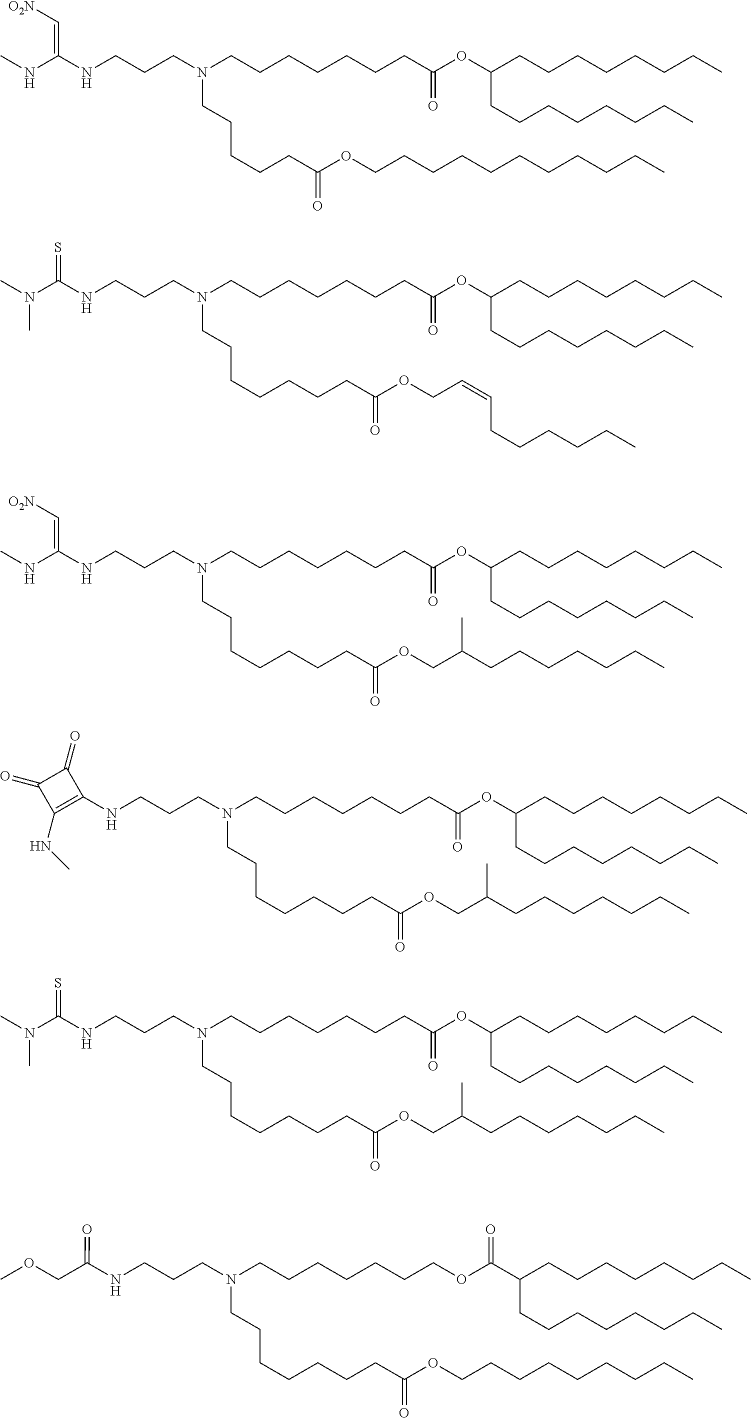

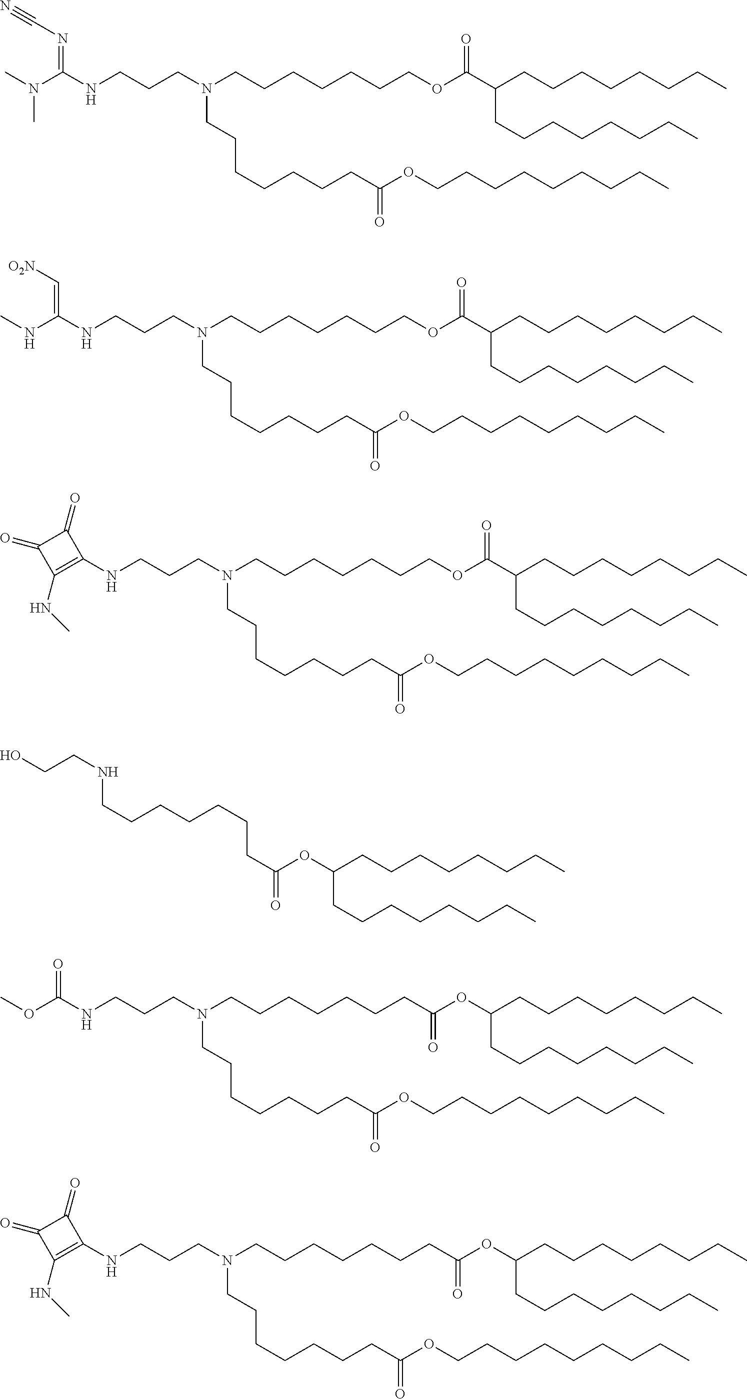

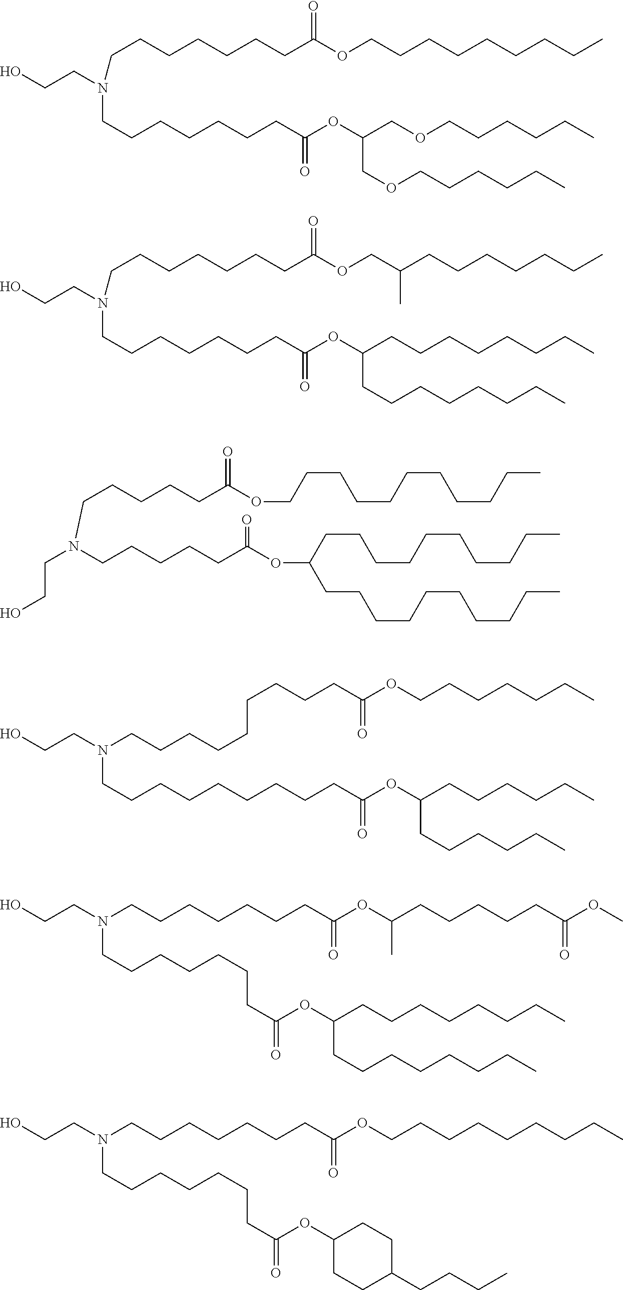 [SMILES: CCCCCCCCCOC(=O)CCCCCCCN(CCO)CCCCCCCC(=O)OC(COCCCCCC)COCCCCCC ; CCCCCCCCC(CCCCCCCC)OC(=O)CCCCCCCN(CCO)CCCCCCCC(=O)OCC(C)CCCCCCC ; CCCCCCCCCCCOC(=O)CCCCCN(CCO)CCCCCC(=O)OC(CCCCCCCCCC)CCCCCCCCCC ; CCCCCCCOC(=O)CCCCCCCCCN(CCO)CCCCCCCCCC(=O)OC(CCCCCC)CCCCCC ; CCCCCCCCC(CCCCCCCC)OC(=O)CCCCCCCN(CCO)CCCCCCCC(=O)OC(C)CCCCCC(=O)OC ; CCCCCCCCCOC(=O)CCCCCCCN(CCO)CCCCCCCC(=O)OC1CCC(CCCC)CC1]

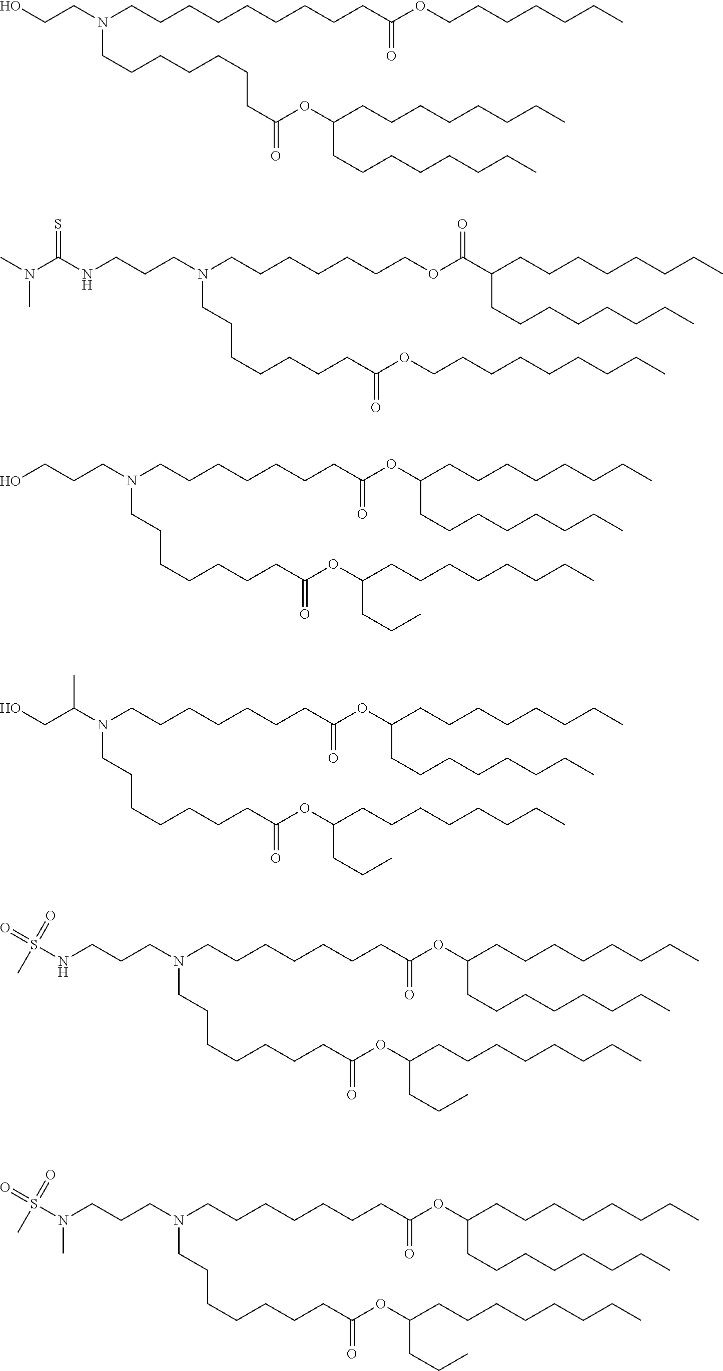

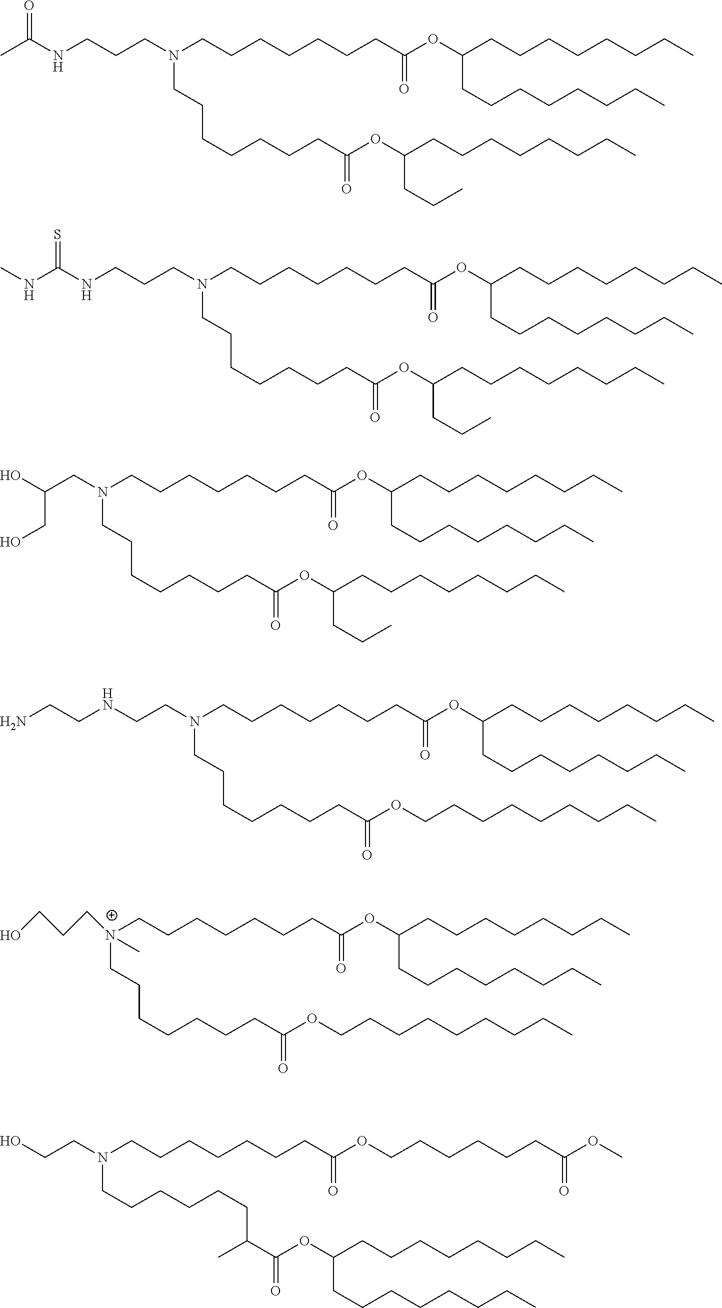

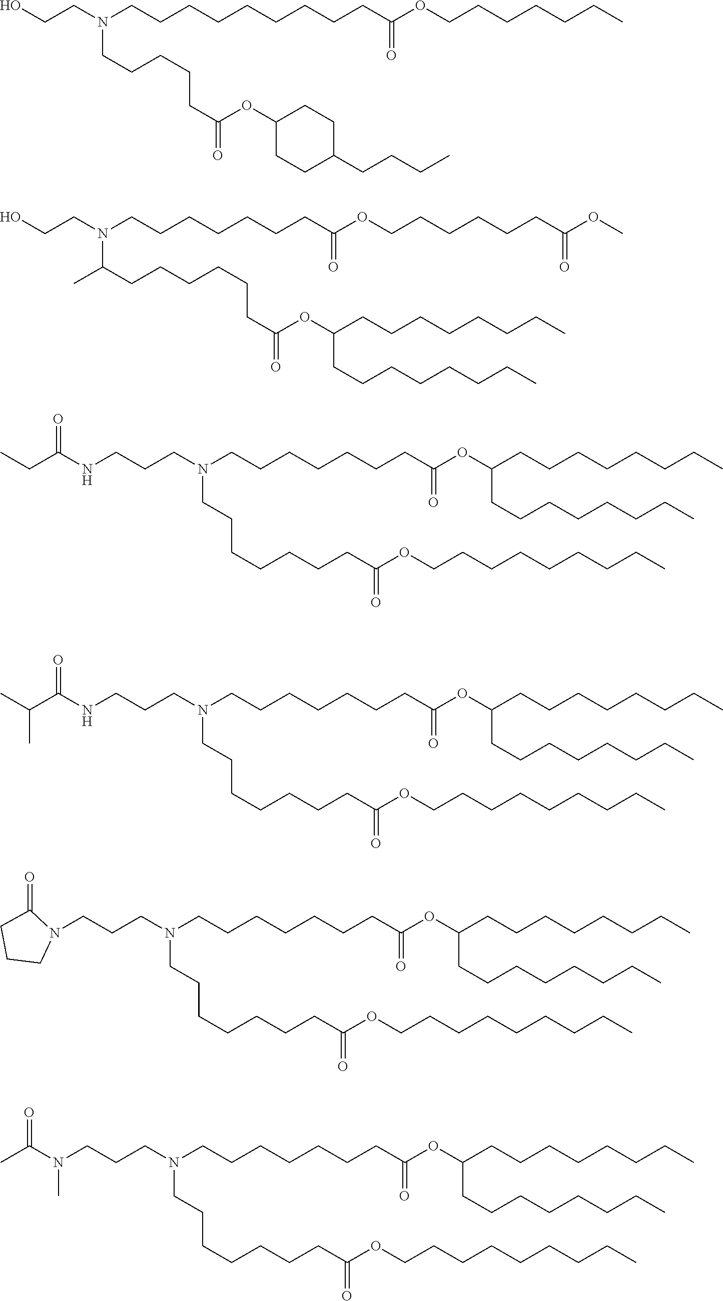

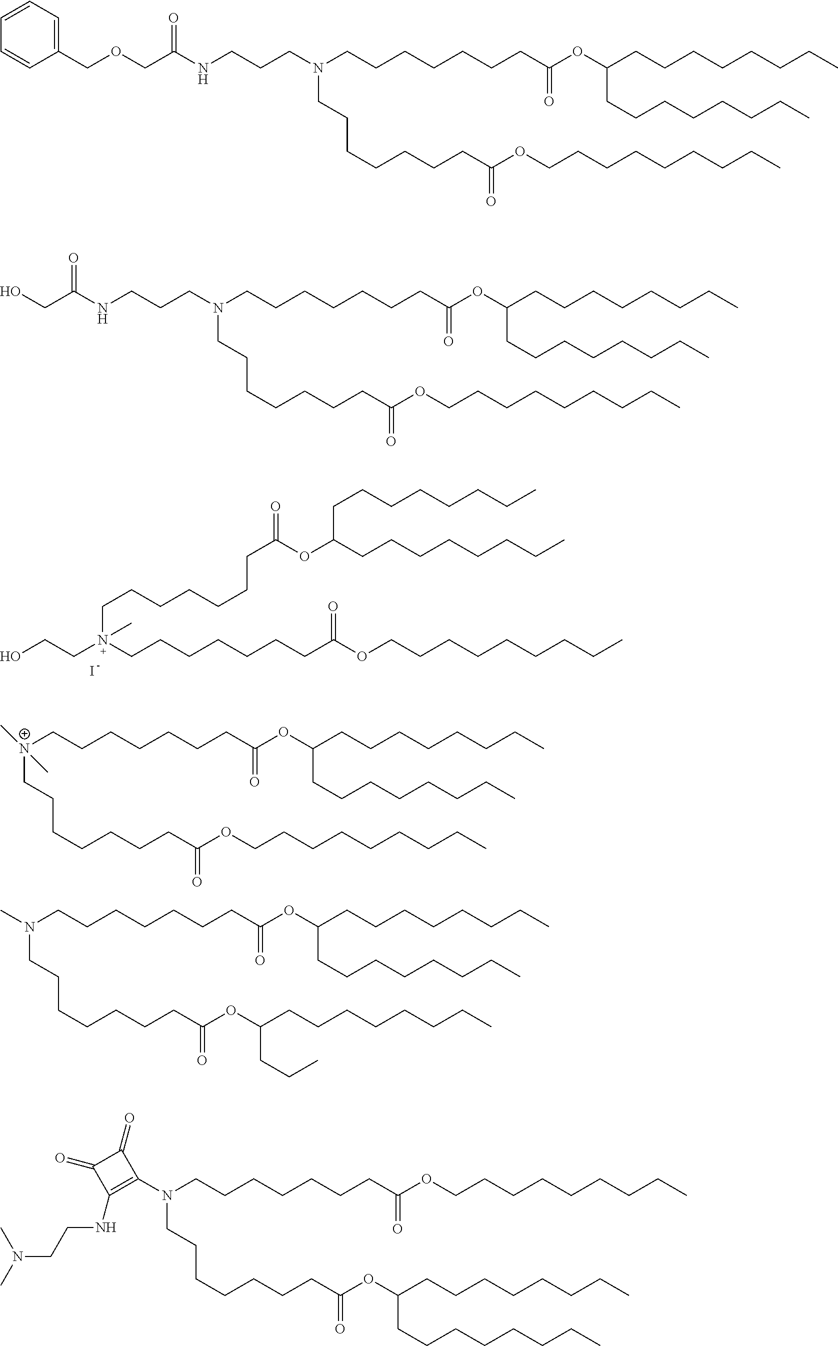

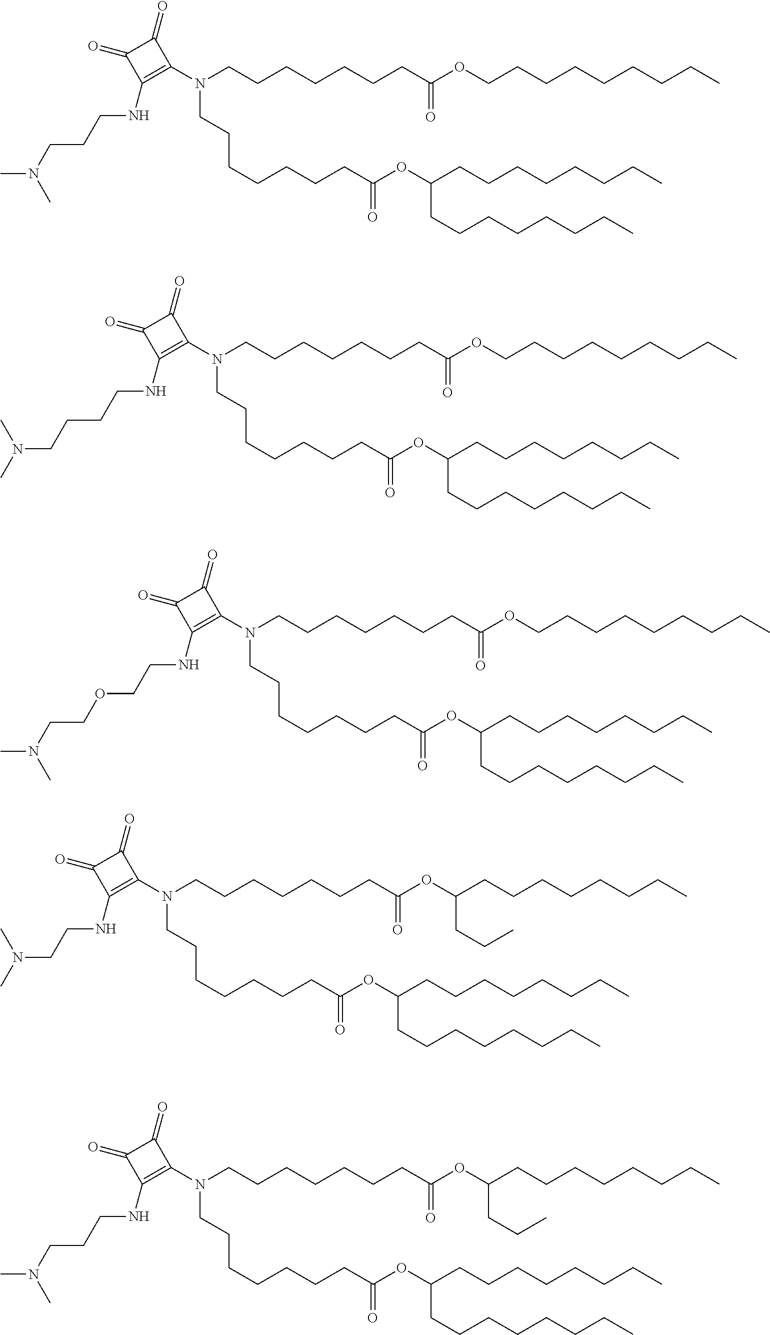

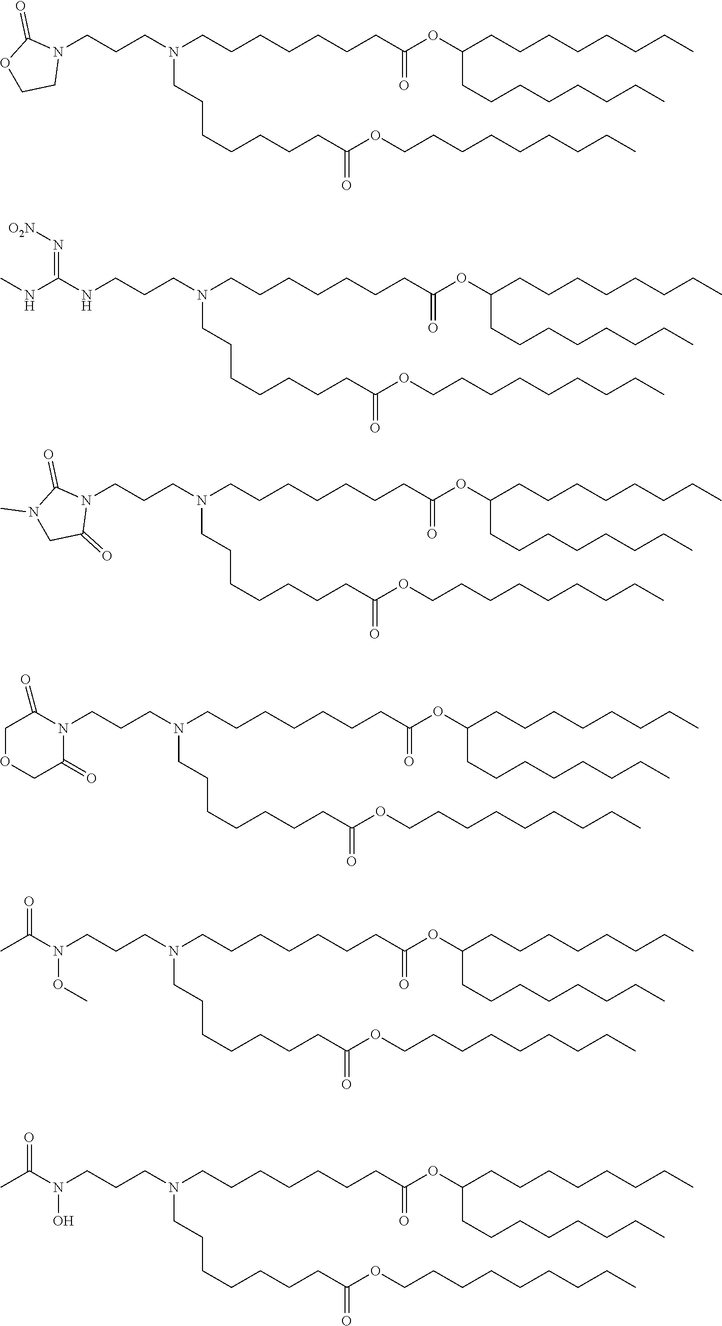

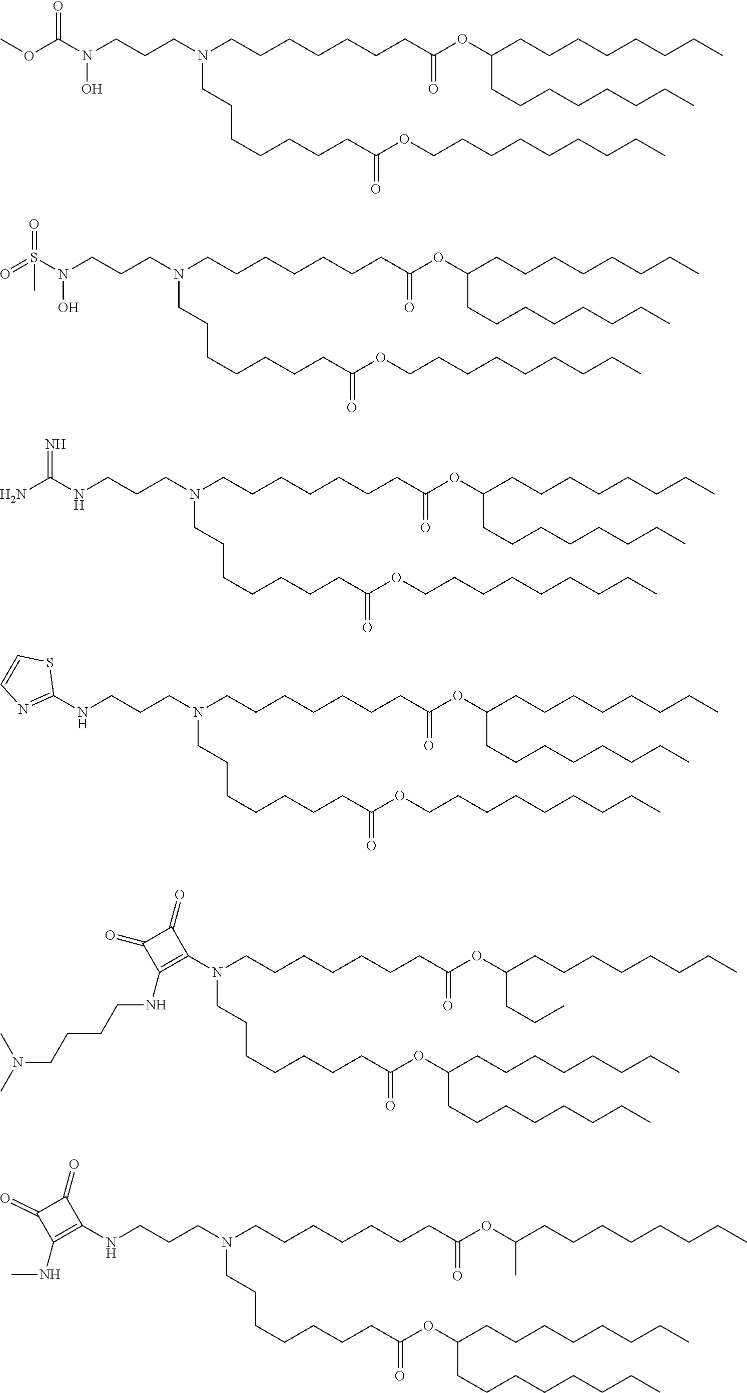

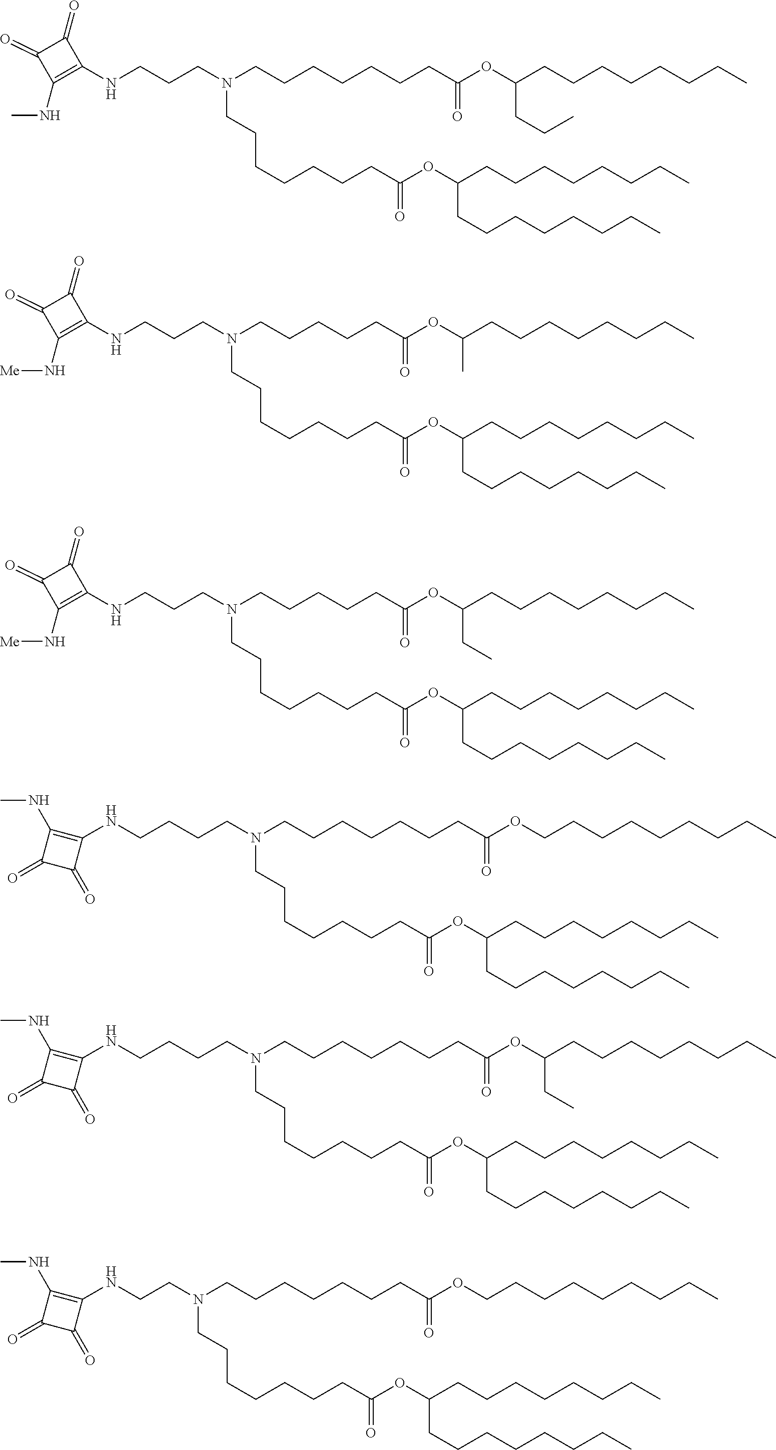

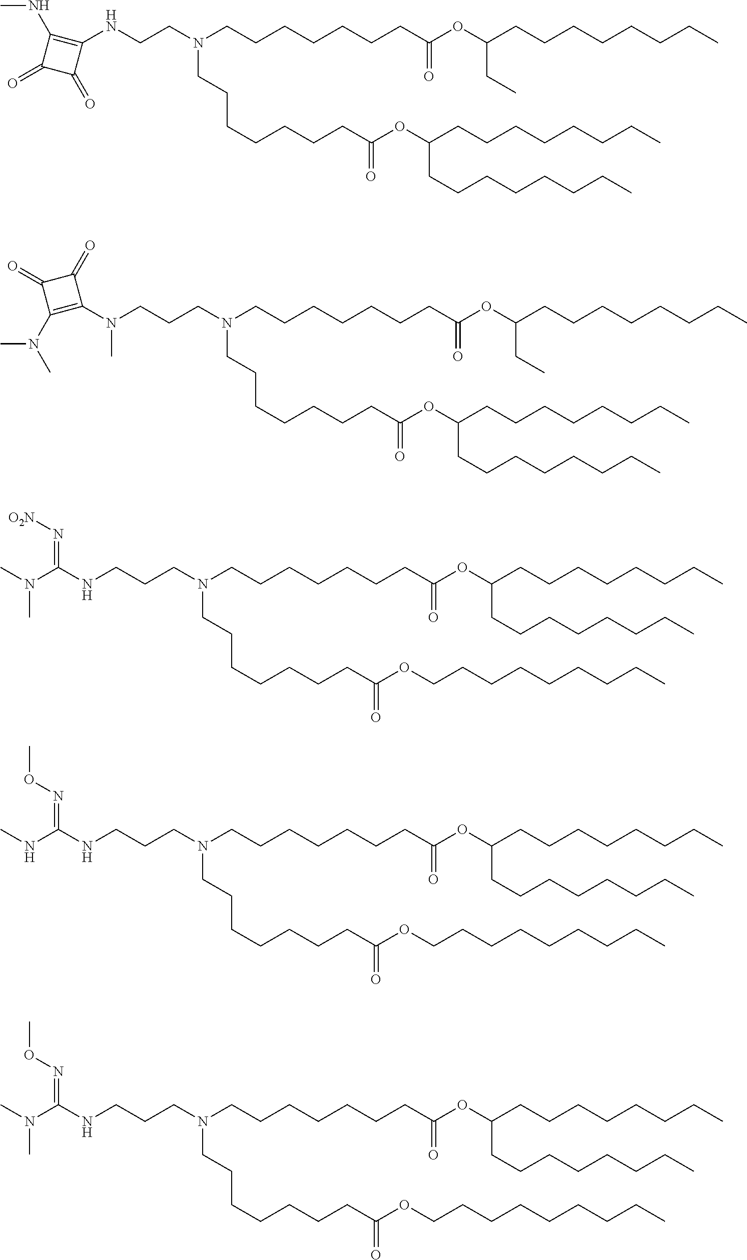

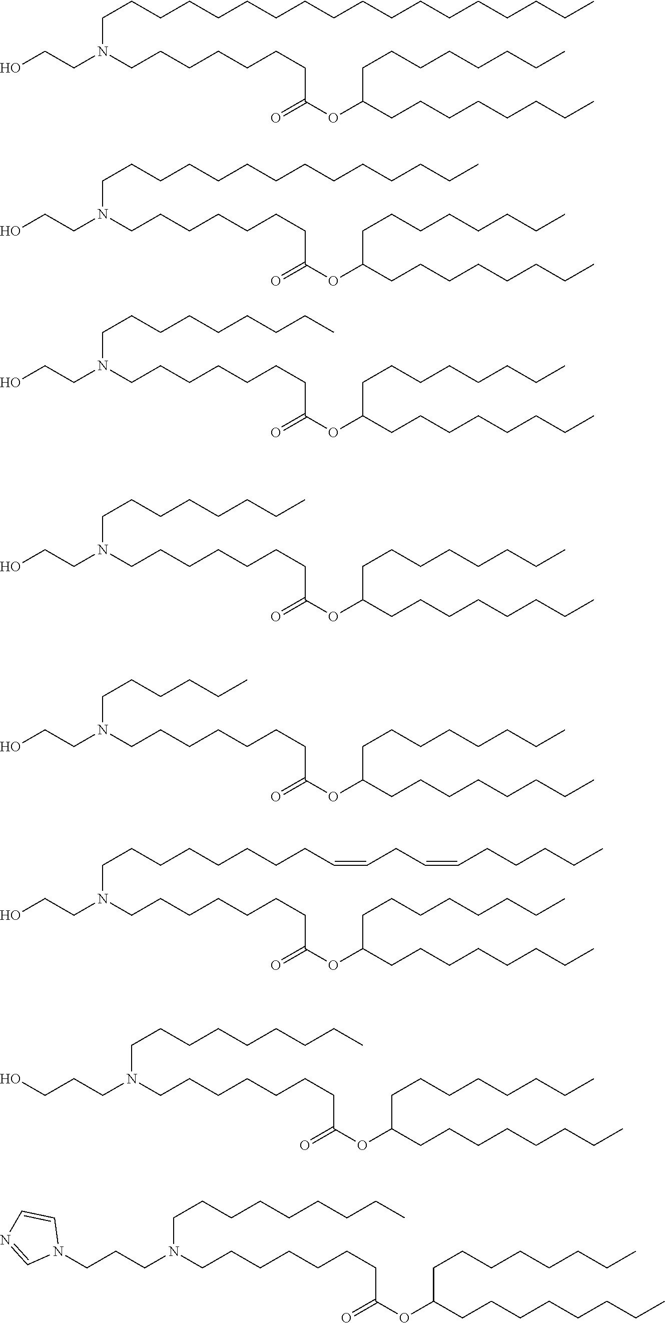

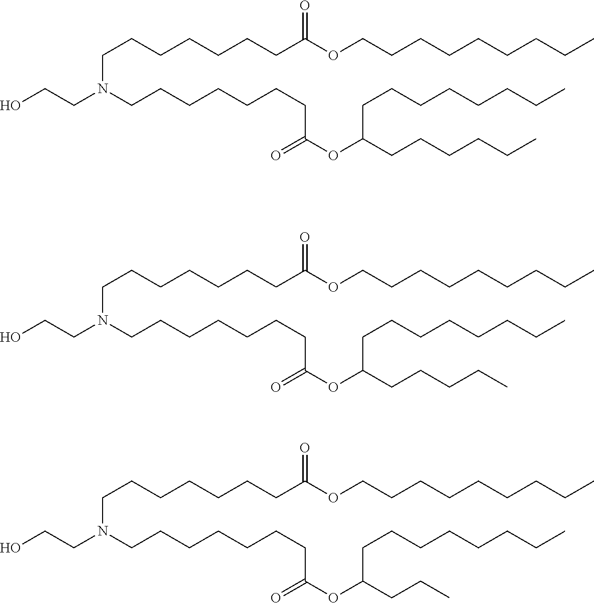 [SMILES: CCCCCCCCCOC(=O)CCCCCCCN(CCO)CCCCCCCC(=O)OC(CCCCCC)CCCCCCCC ; CCCCCCCCCOC(=O)CCCCCCCN(CCO)CCCCCCCC(=O)OC(CCCCC)CCCCCCCC ; CCCCCCCCCOC(=O)CCCCCCCN(CCO)CCCCCCCC(=O)OC(CCC)CCCCCCCC]

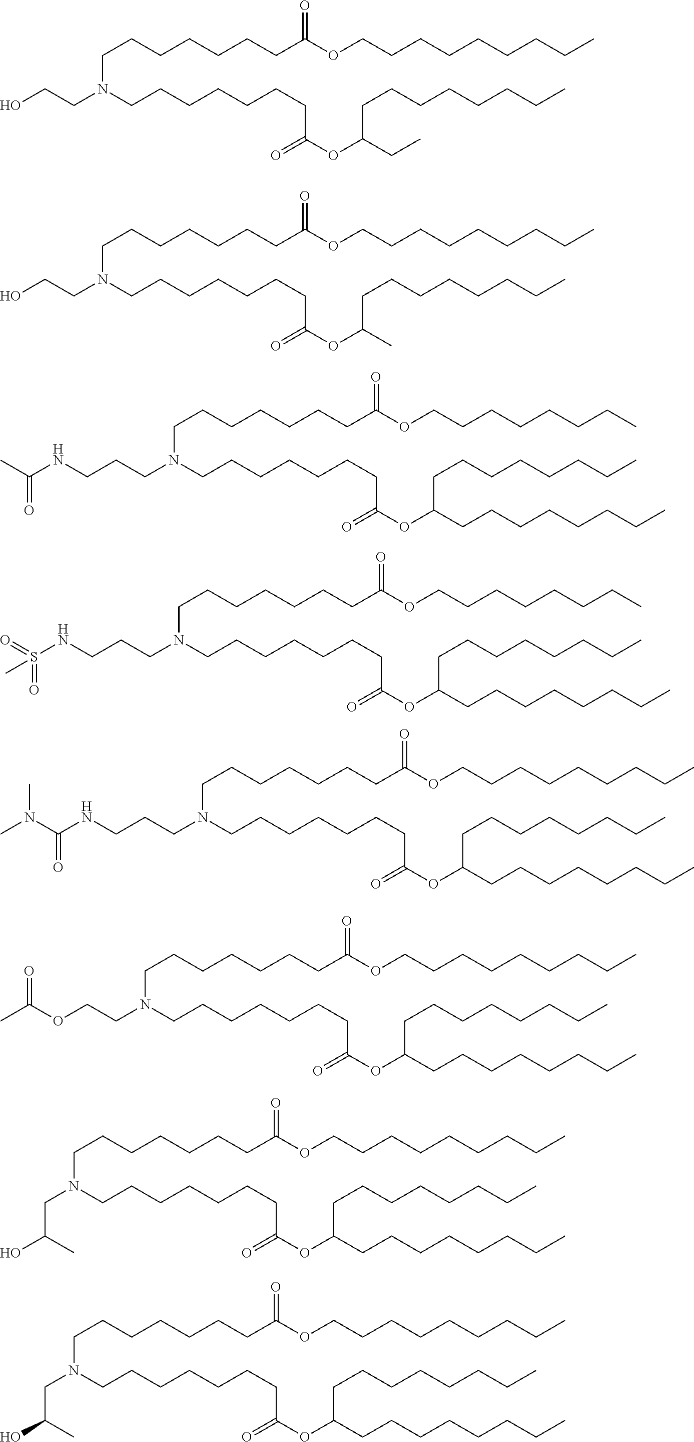

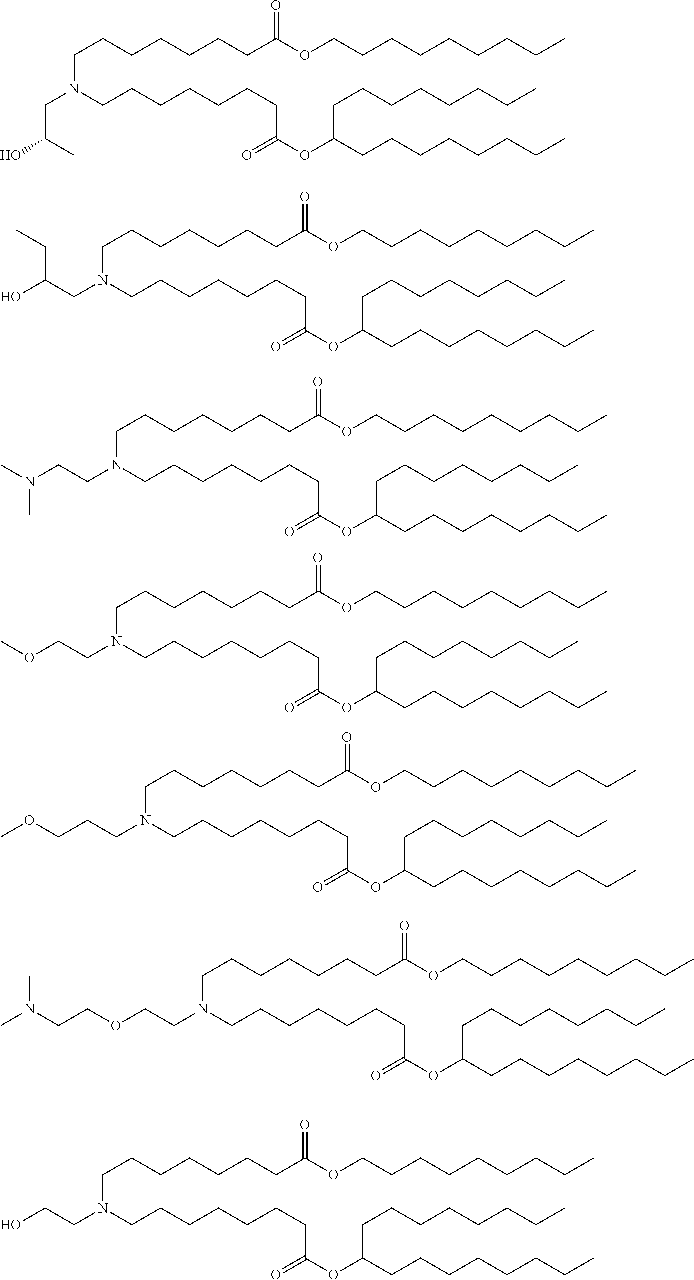

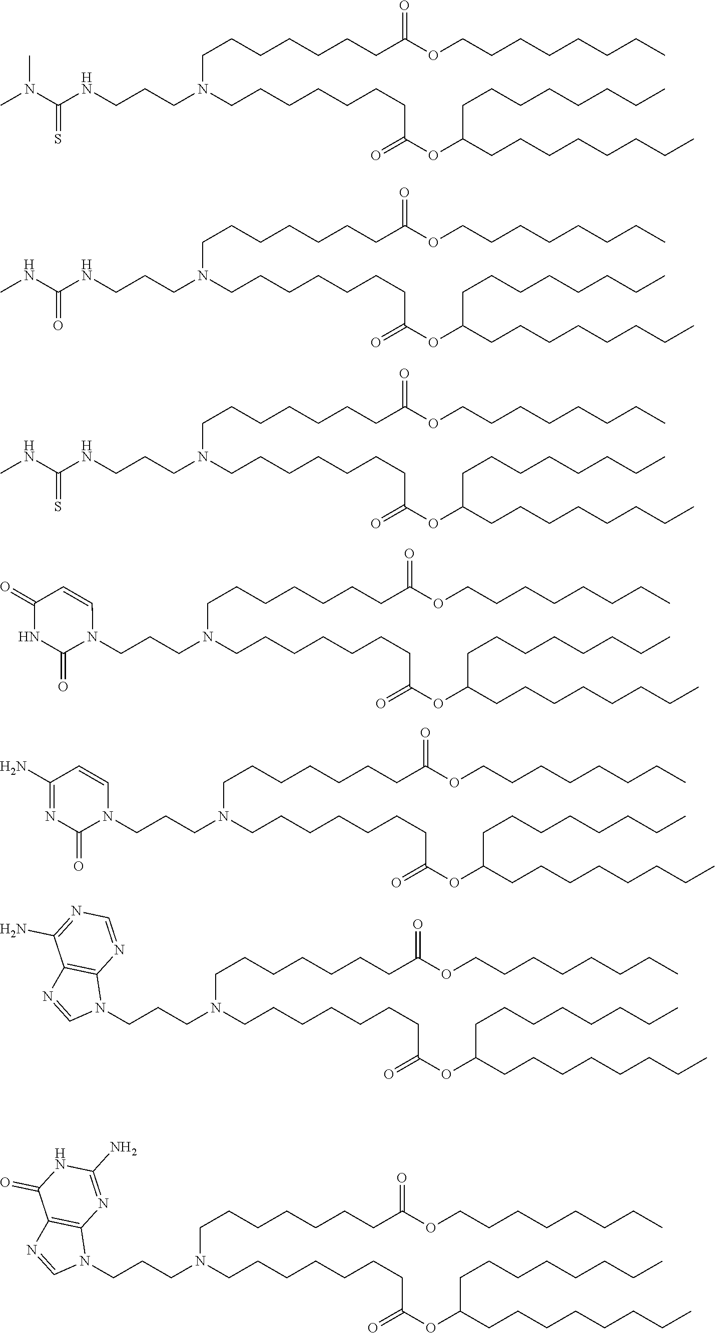

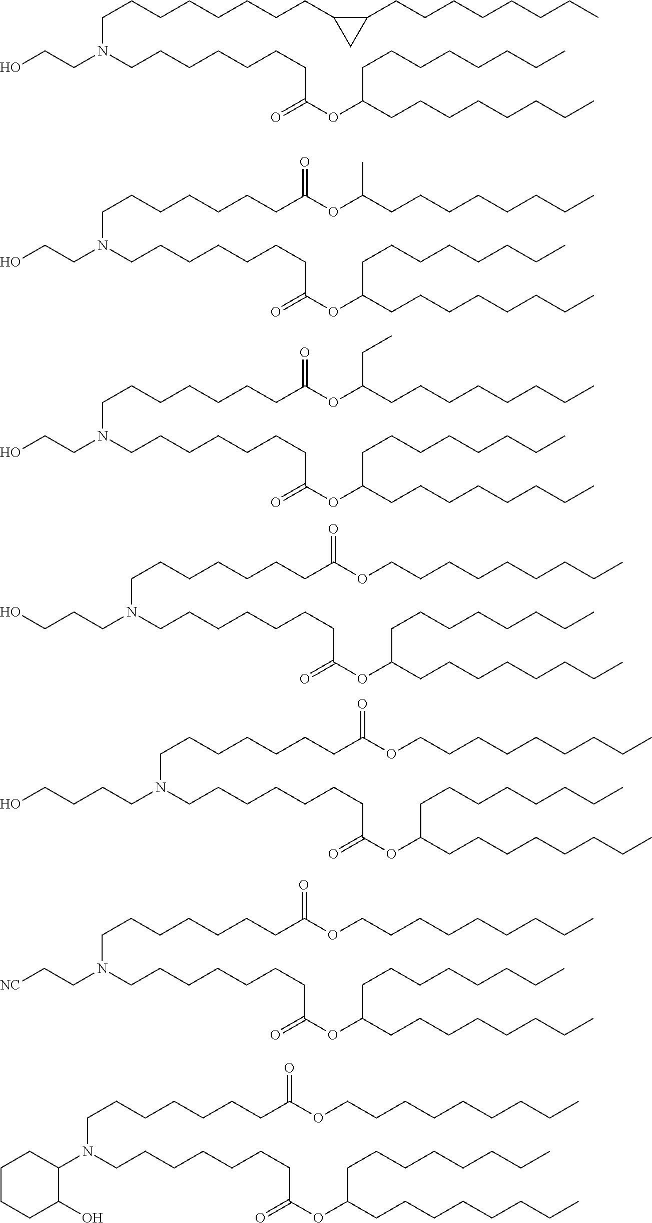

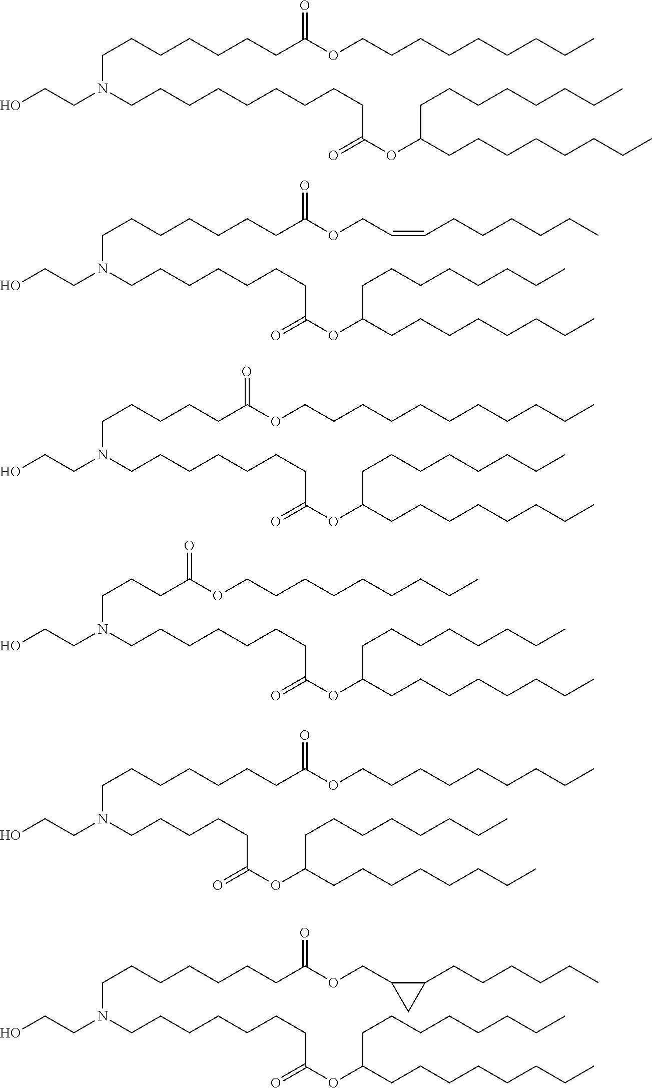

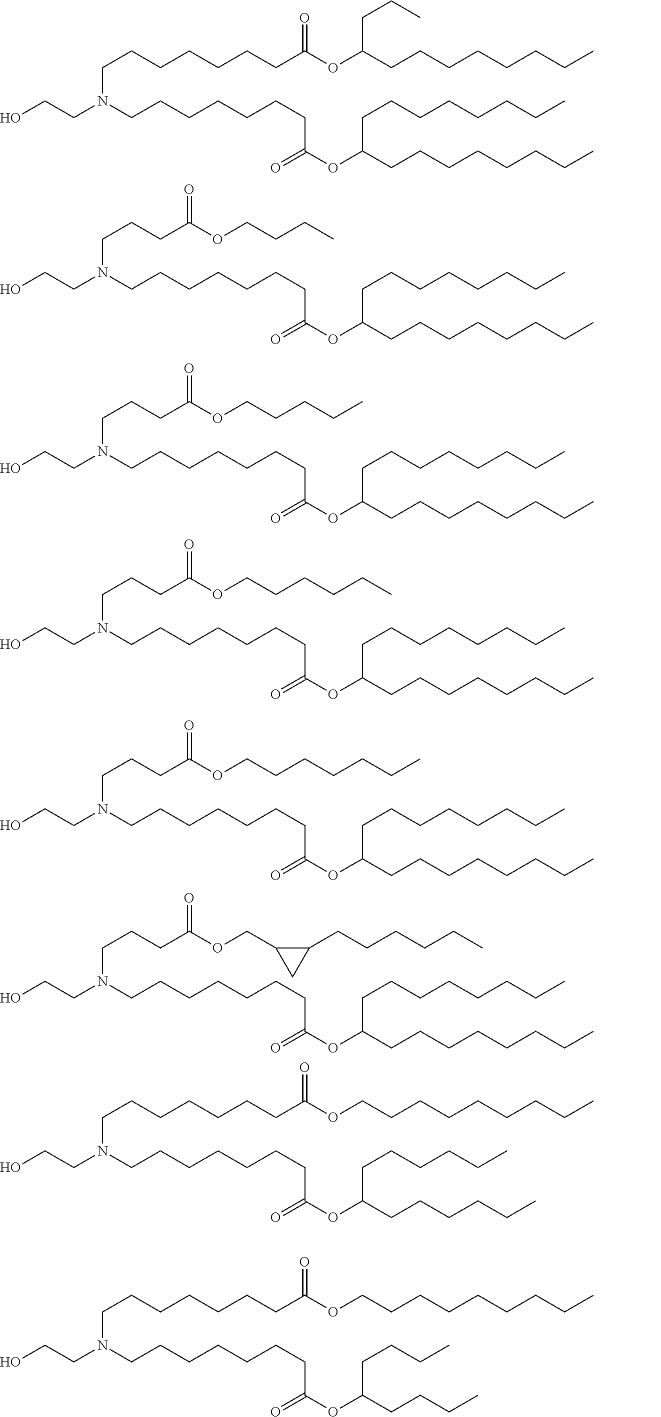

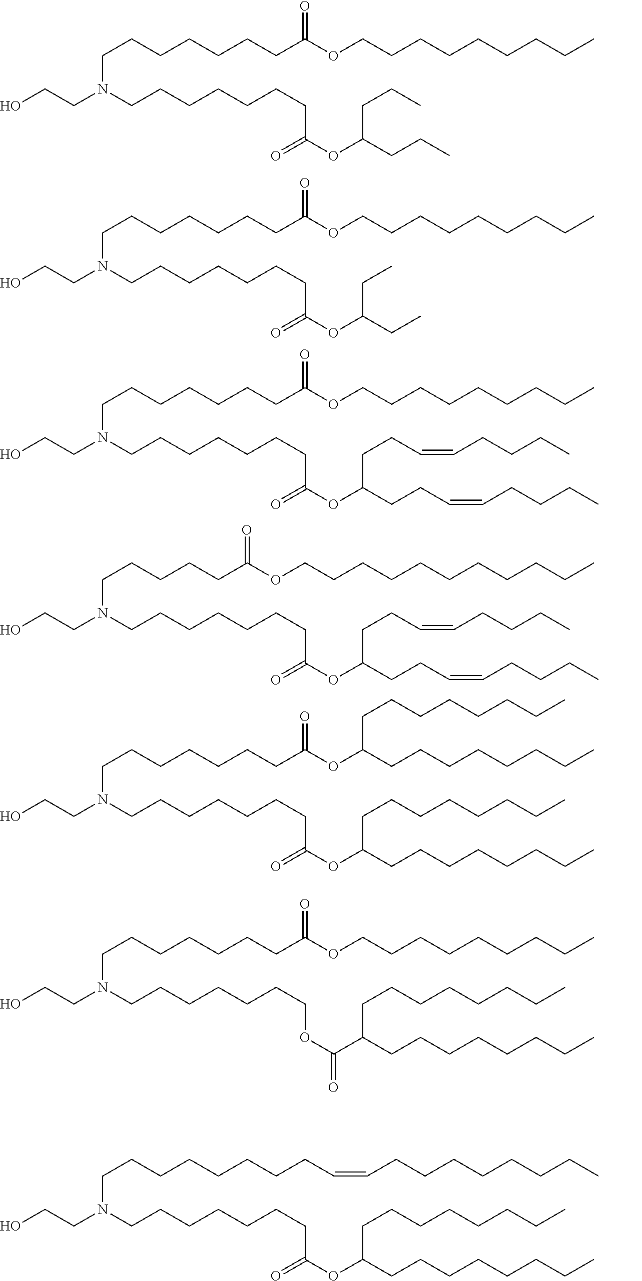

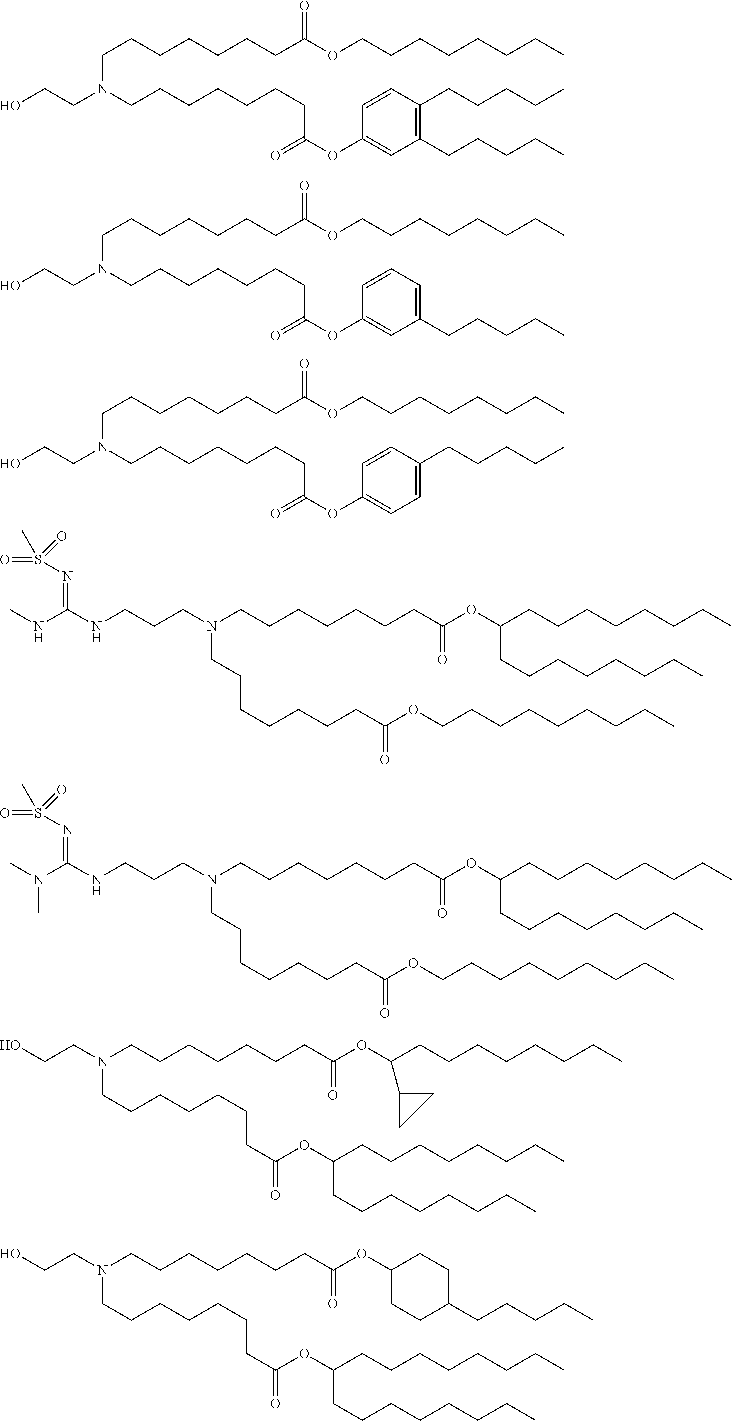

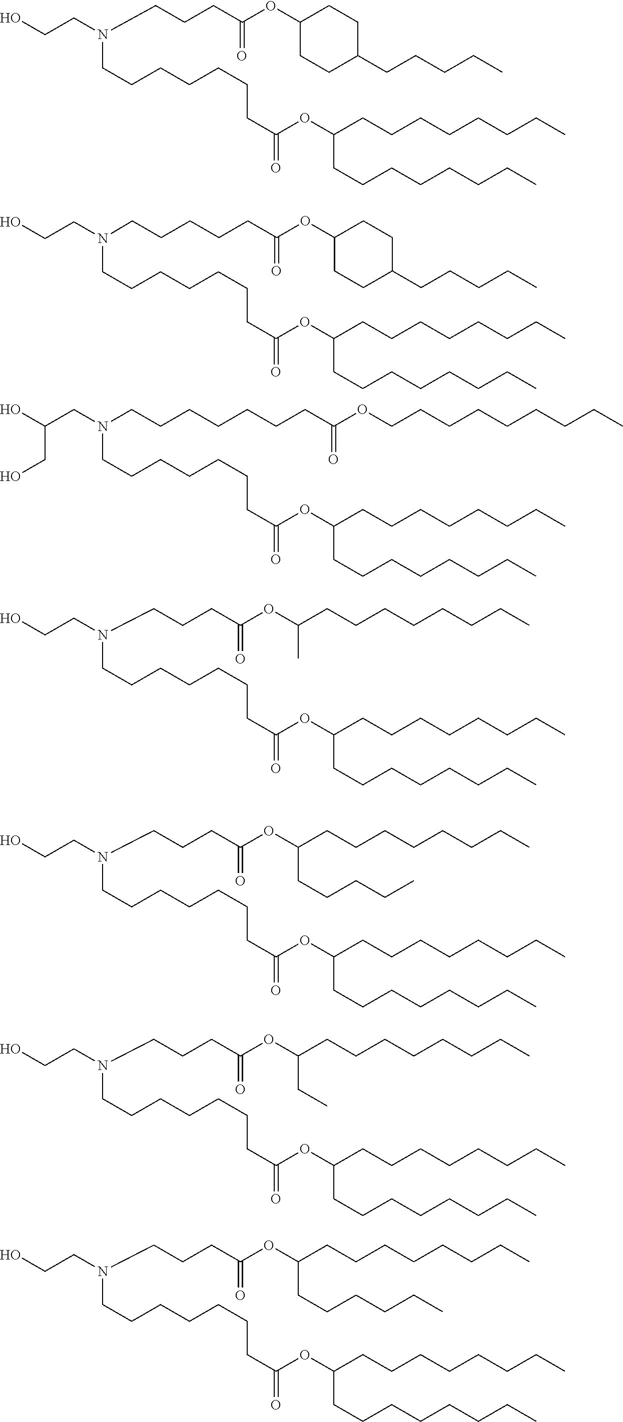

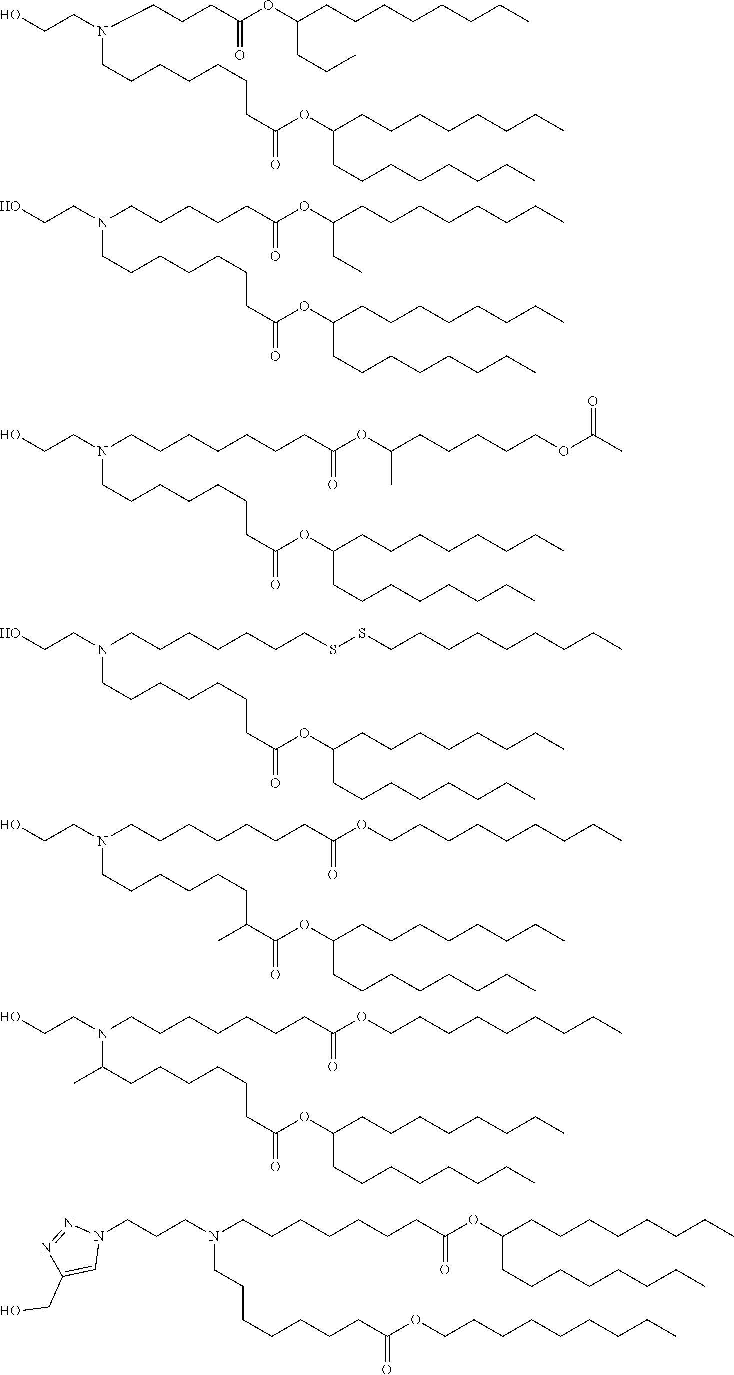

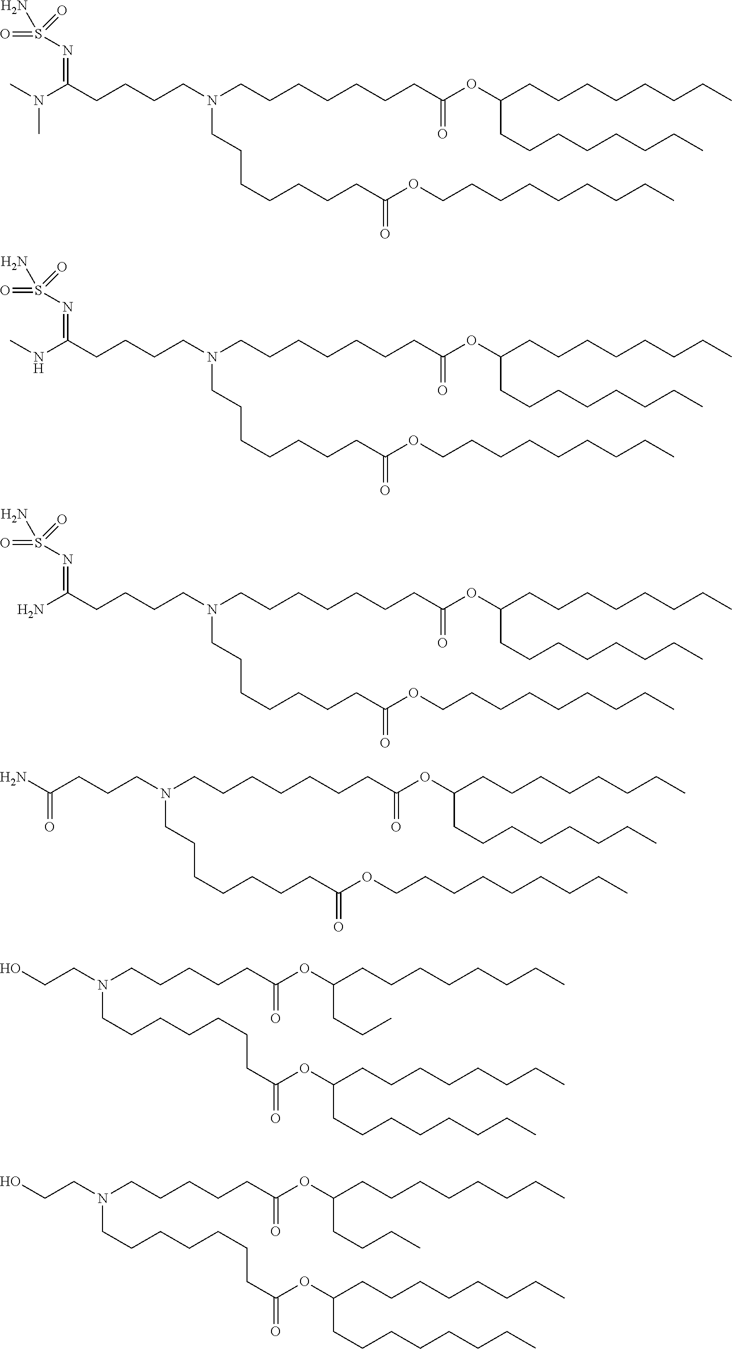

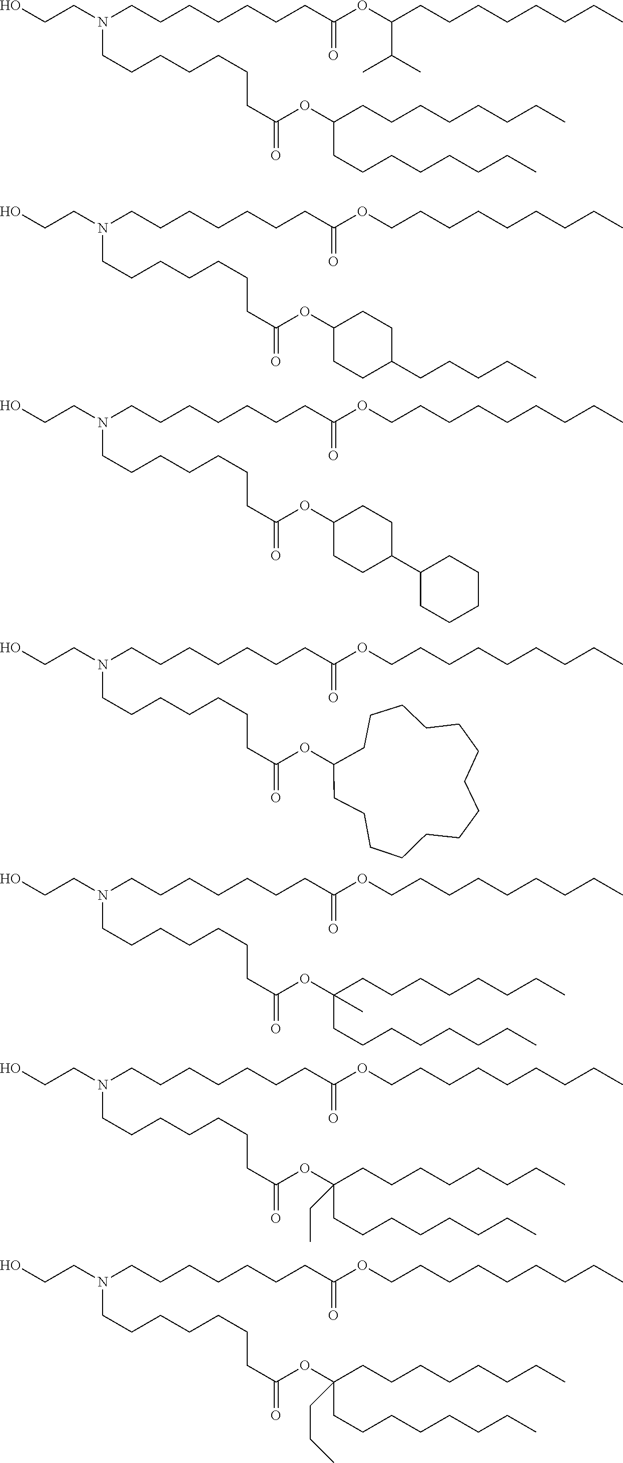

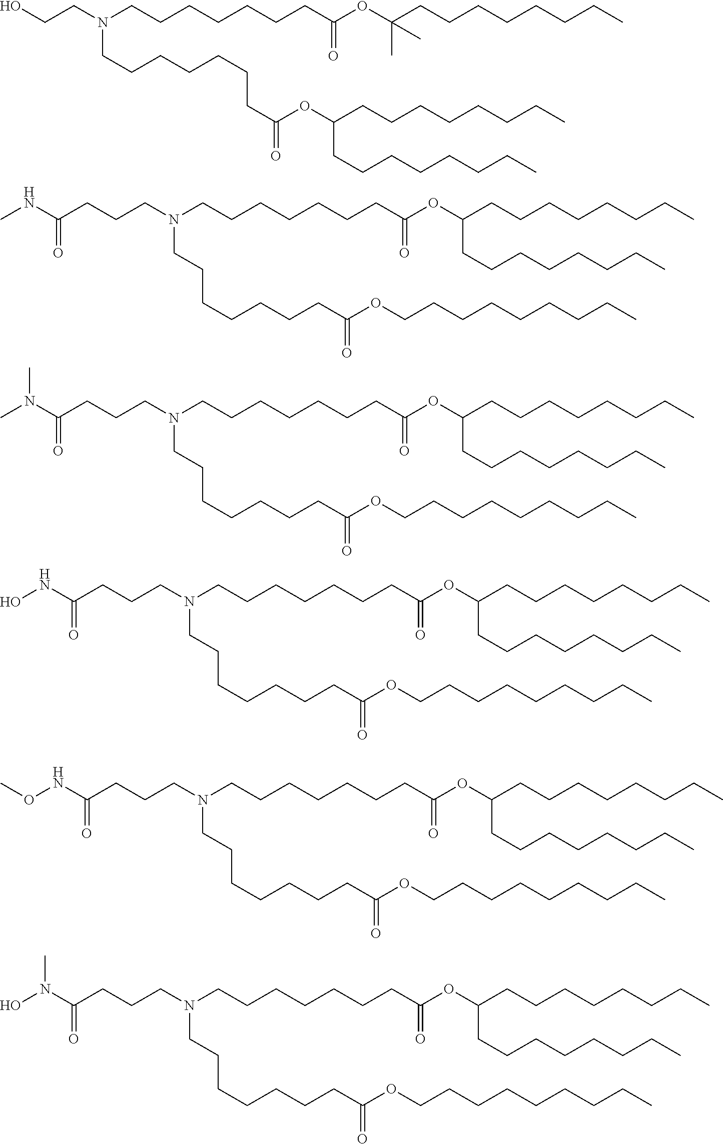

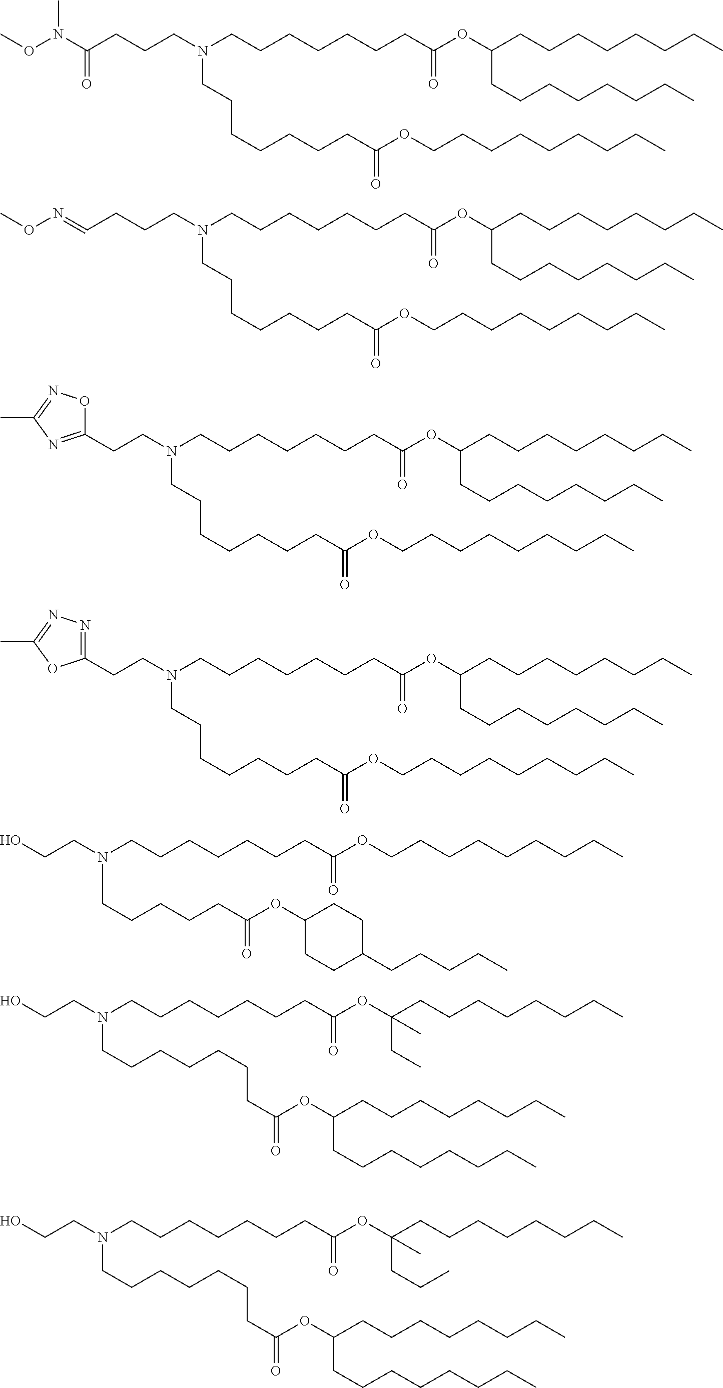

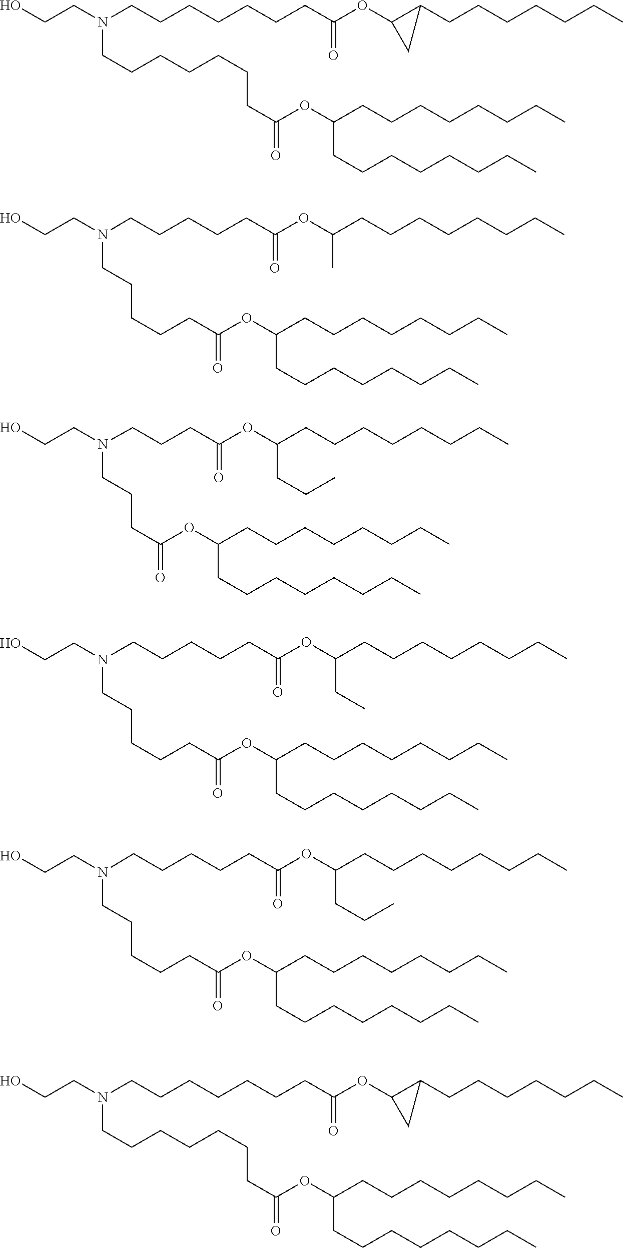

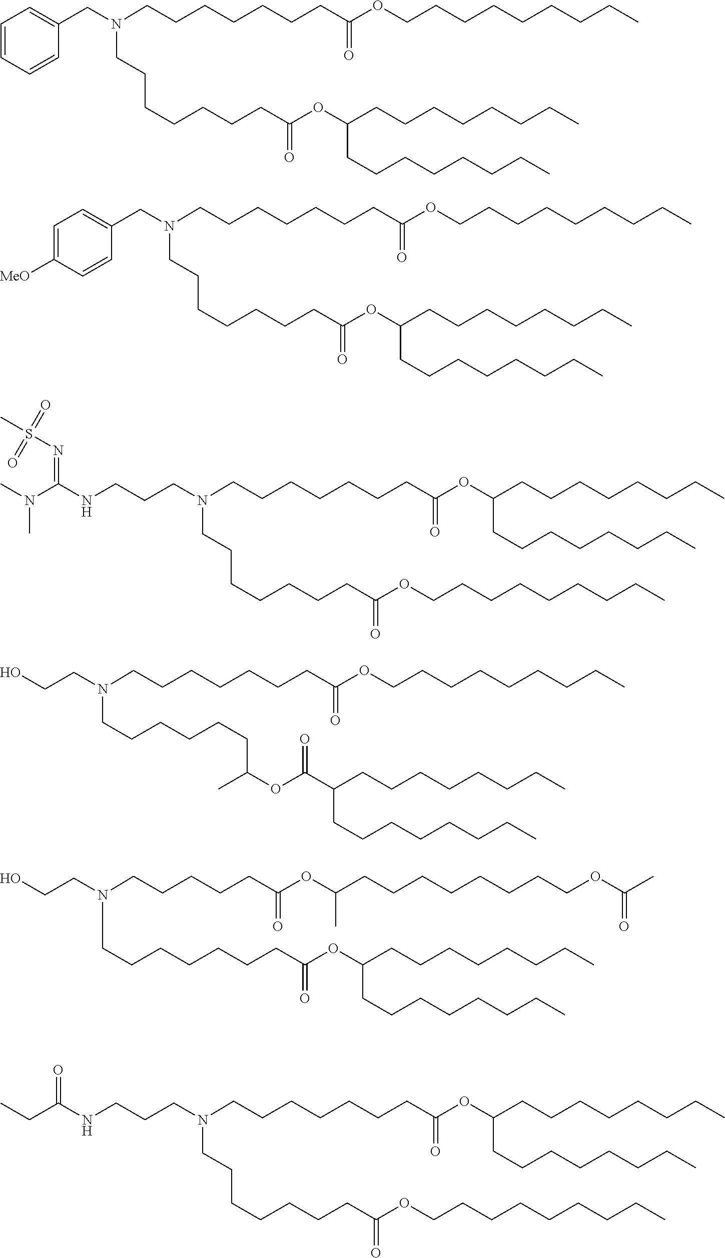

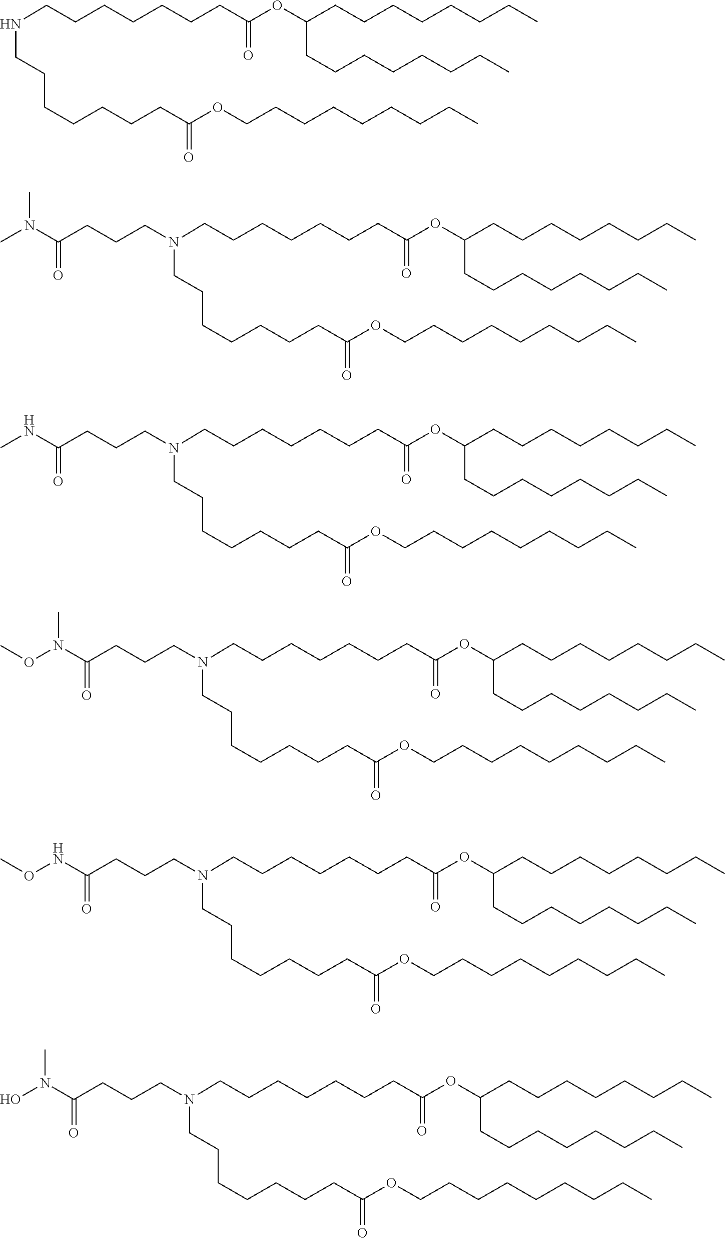

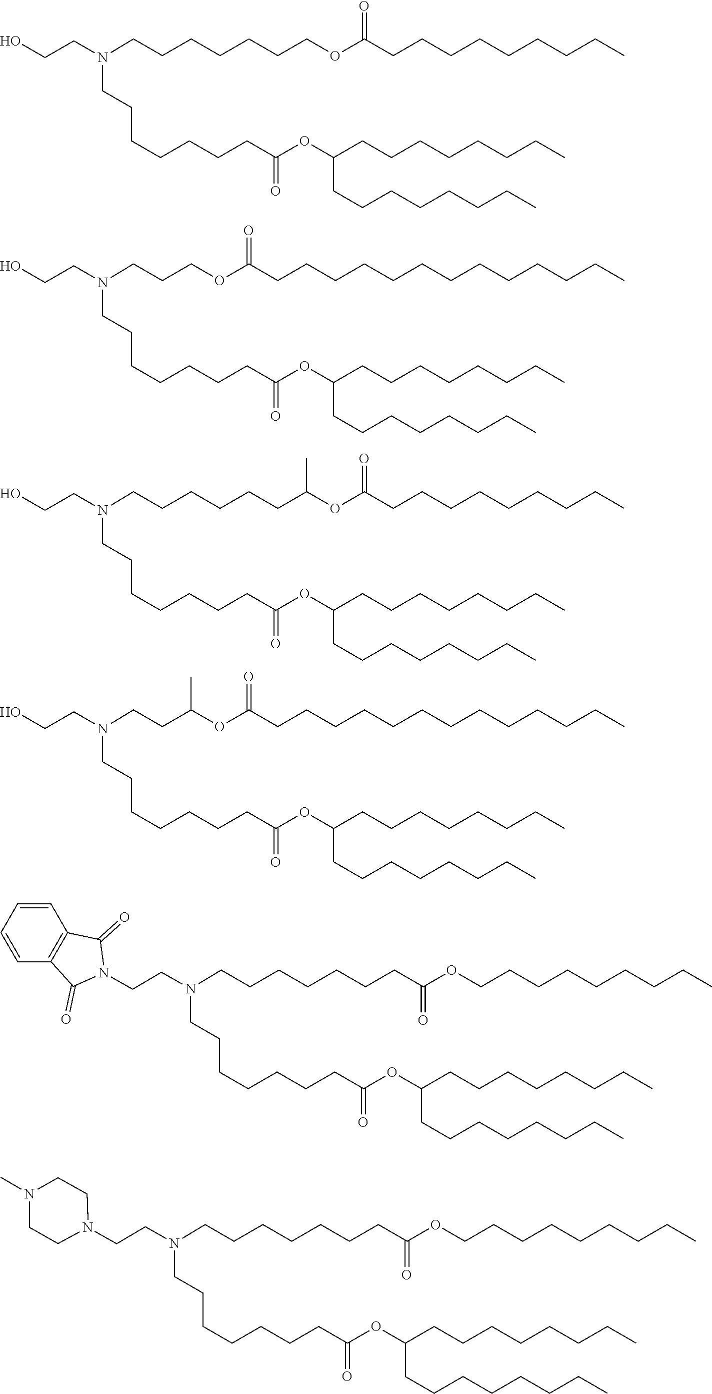

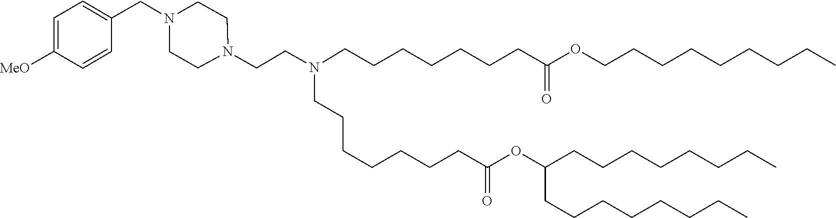 [SMILES: CCCCCCCCCOC(=O)CCCCCCCN(CCCCCCCC(=O)OC(CCCCCCCC)CCCCCCCC)CCN1CCN(Cc2ccc(OC)cc2)CC1]

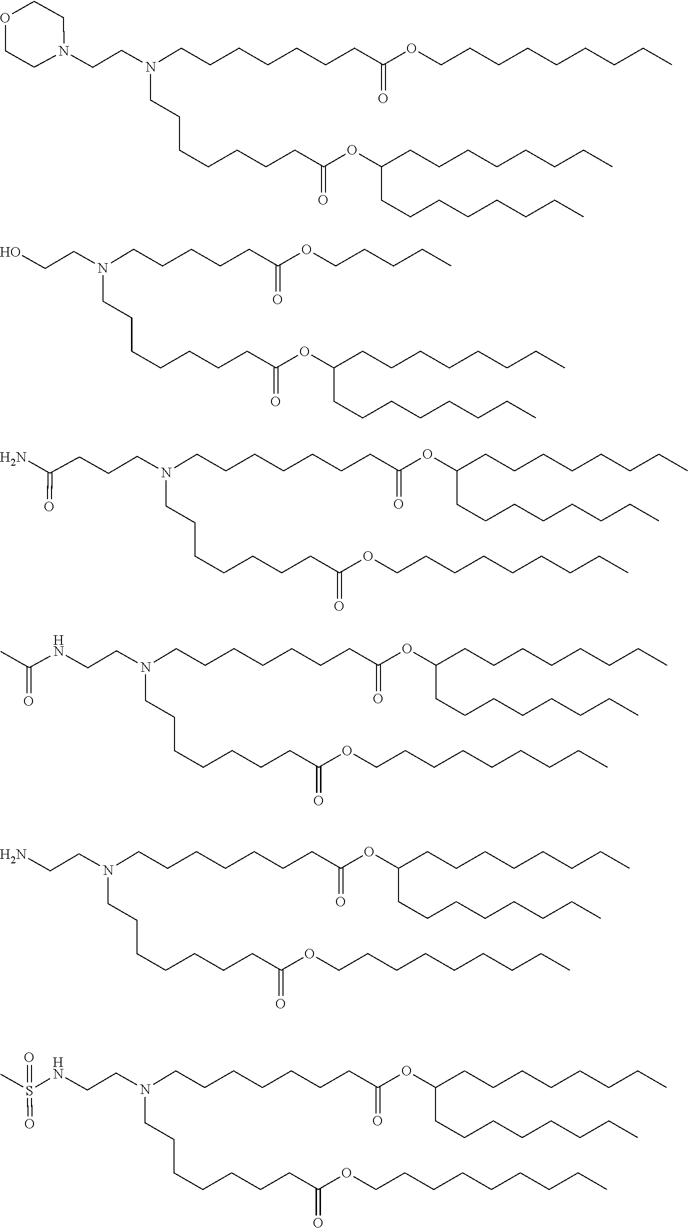

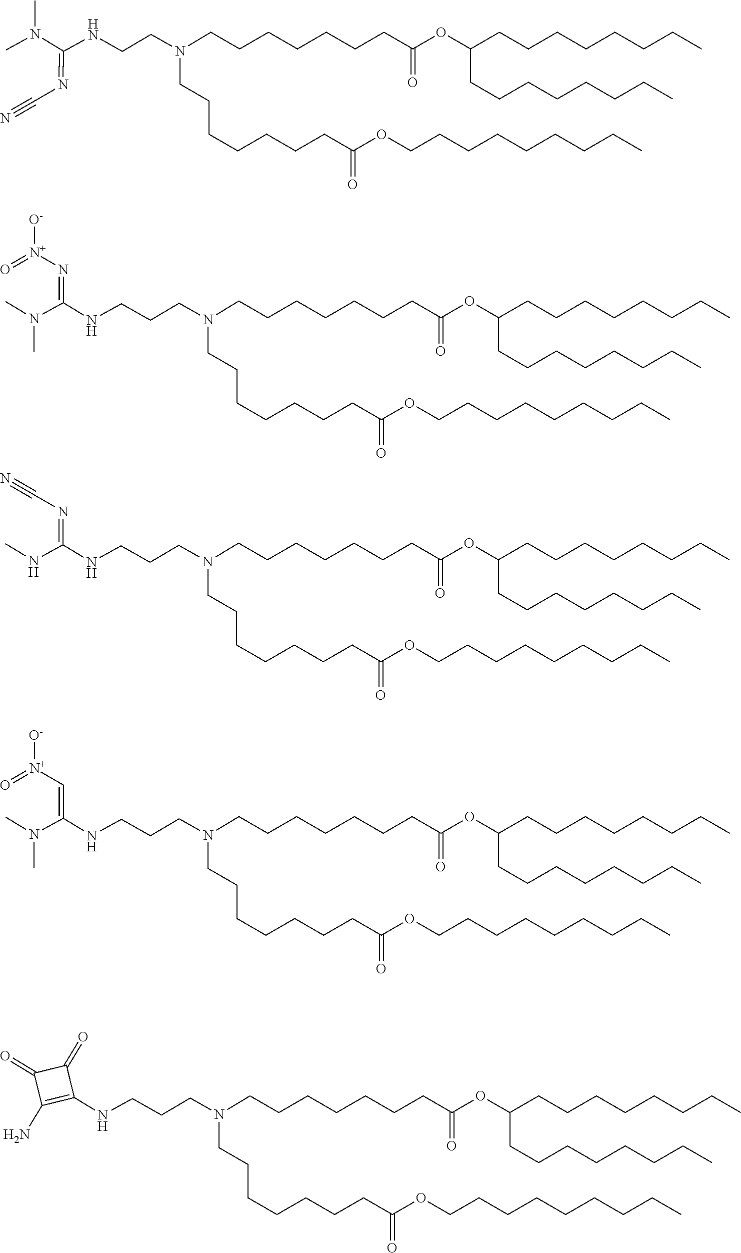 [SMILES: CCCCCCCCCOC(=O)CCCCCCCN(CCCCCCCC(=O)OC(CCCCCCCC)CCCCCCCC)CCN/C(=N/C#N)N(C)C ; CCCCCCCCCOC(=O)CCCCCCCN(CCCCCCCC(=O)OC(CCCCCCCC)CCCCCCCC)CCCN/C(=N/[N+](=O)[O-])N(C)C ; CCCCCCCCCOC(=O)CCCCCCCN(CCCCCCCC(=O)OC(CCCCCCCC)CCCCCCCC)CCCN/C(=N/C#N)NC ; CCCCCCCCCOC(=O)CCCCCCCN(CCCCCCCC(=O)OC(CCCCCCCC)CCCCCCCC)CCCN/C(=C/[N+](=O)[O-])N(C)C ; CCCCCCCCCOC(=O)CCCCCCCN(CCCCCCCC(=O)OC(CCCCCCCC)CCCCCCCC)CCCNc1c(N)c(=O)c1=O]

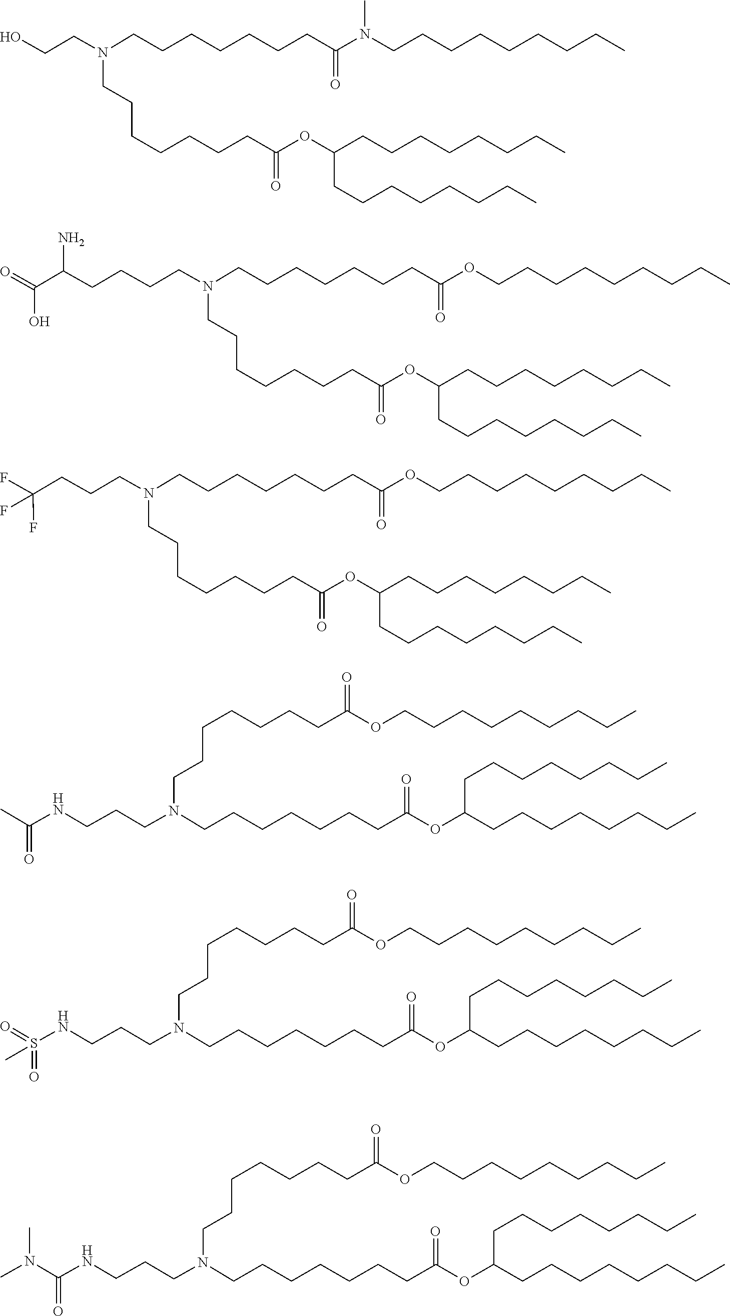

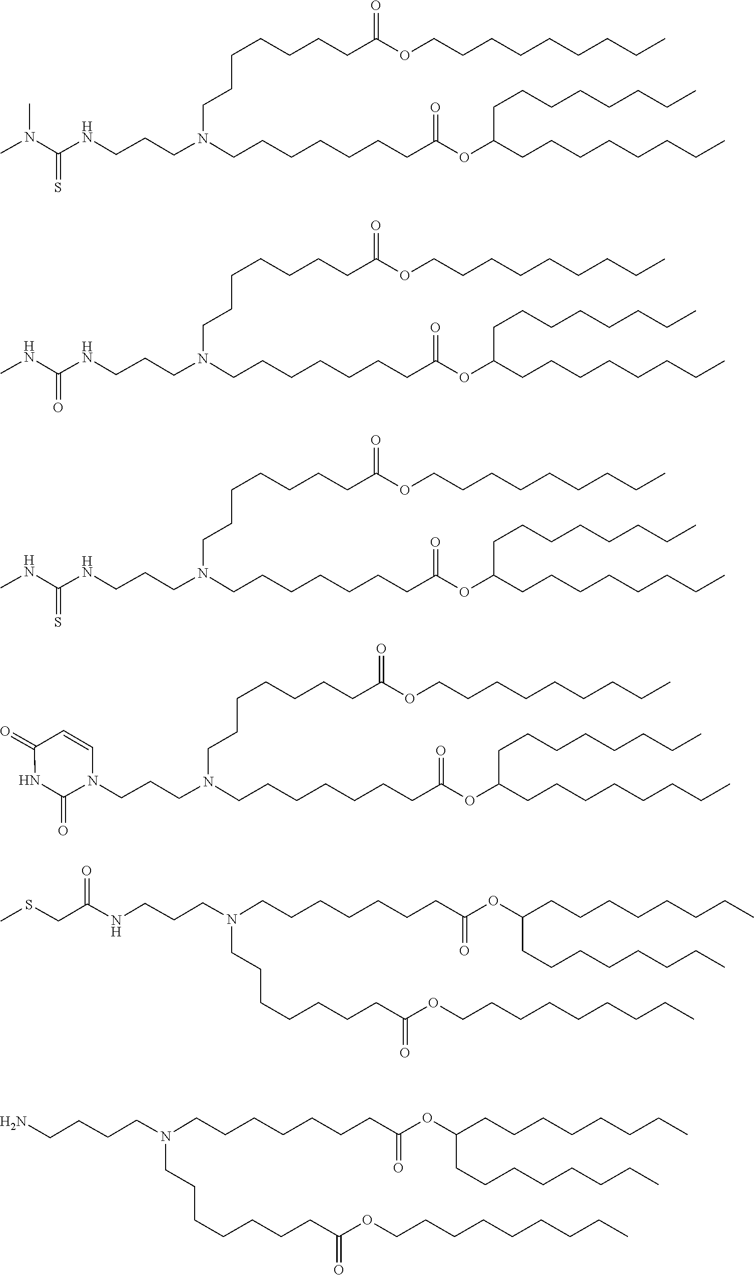

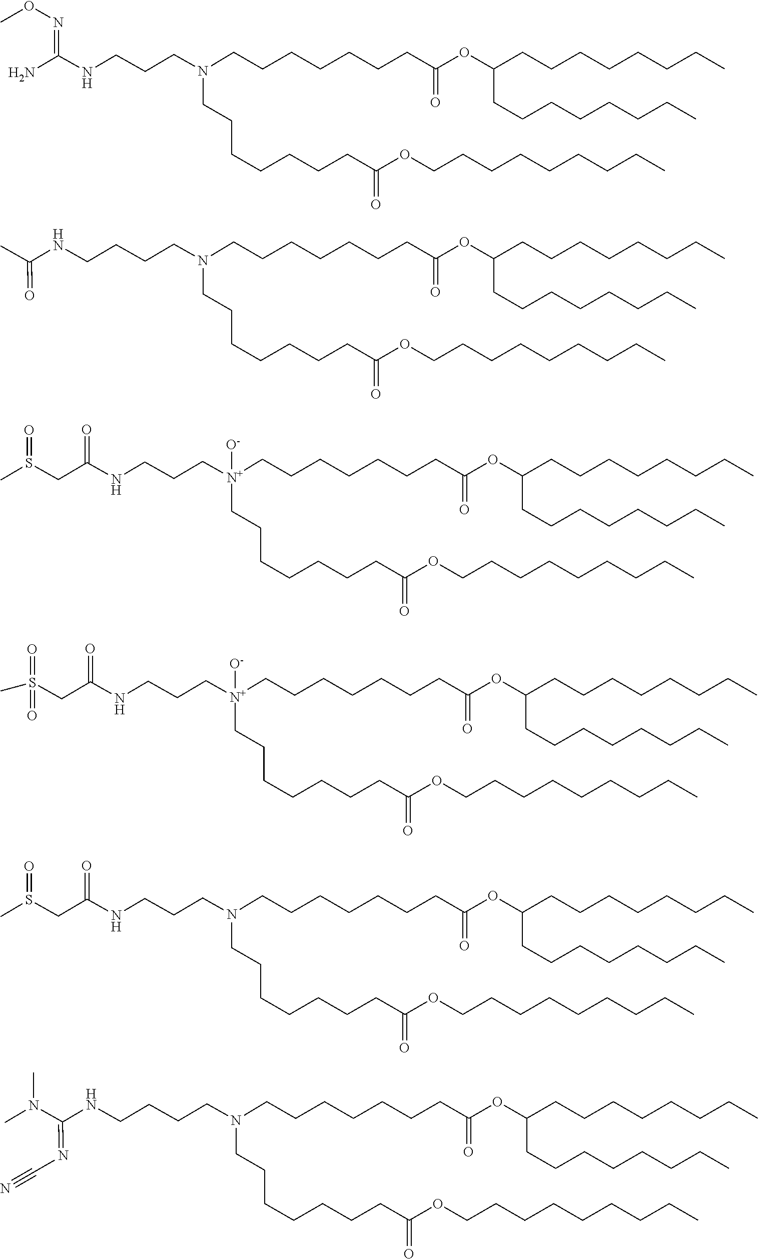

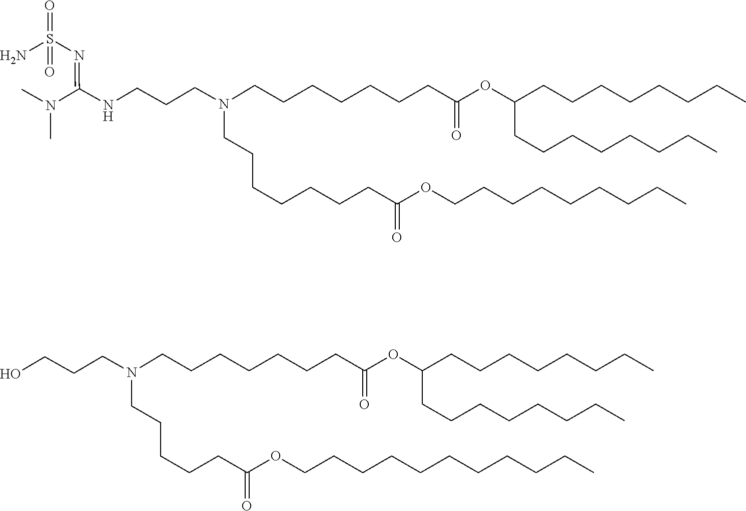 [SMILES: CCCCCCCCCOC(=O)CCCCCCCN(CCCCCCCC(=O)OC(CCCCCCCC)CCCCCCCC)CCCN/C(=N/S(N)(=O)=O)N(C)C ; CCCCCCCCCCCOC(=O)CCCCCN(CCCO)CCCCCCCC(=O)OC(CCCCCCCC)CCCCCCCC]

### 5.1 Amine Lipids

In certain embodiments, transfer vehicle compositions for the delivery of circular RNA comprise an amine lipid. In certain embodiments, an ionizable lipid is an amine lipid. In some embodiments, an amine lipid is described in international patent application PCT/US2018/053569.

In some embodiments, the amine lipid is Lipid E, which is (9Z, 12Z)-3-((4,4-bis(octyloxy)butanoyl)oxy)-2-((((3-(diethylamino)propoxy)carbonyl)oxy)methyl)propyl octadeca-9, 12-dienoate.

Lipid E can be depicted as:

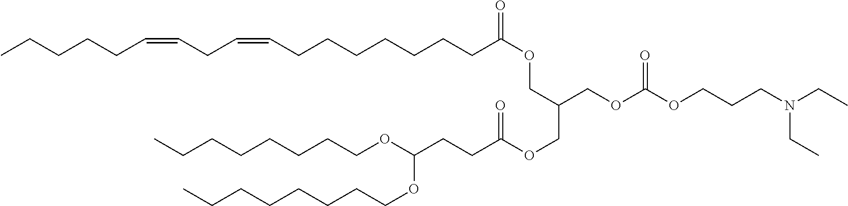 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid E may be synthesized according to WO2015/095340 (e.g., pp. 84-86). In certain embodiments, the amine lipid is an equivalent to Lipid E.

In certain embodiments, an amine lipid is an analog of Lipid E. In certain embodiments, a Lipid E analog is an acetal analog of Lipid E. In particular transfer vehicle compositions, the acetal analog is a C4-C12 acetal analog. In some embodiments, the acetal analog is a C5-C12 acetal analog. In additional embodiments, the acetal analog is a C5-C10 acetal analog. In further embodiments, the acetal analog is chosen from a C4, C5, C6, C7, C9, C10, C11 and C12 acetal analog.

Amine lipids and other biodegradable lipids suitable for use in the transfer vehicles, e.g., lipid nanoparticles, described herein are biodegradable in vivo. The amine lipids described herein have low toxicity (e.g., are tolerated in animal models without adverse effect in amounts of greater than or equal to 10 mg/kg). In certain embodiments, transfer vehicles composing an amine lipid include those where at least 75% of the amine lipid is cleared from the plasma within 8, 10, 12, 24, or 48 hours, or 3, 4, 5, 6, 7, or 10 days.

Biodegradable lipids include, for example, the biodegradable lipids of WO2017/173054, WO2015/095340, and WO2014/136086.

Lipid clearance may be measured by methods known by persons of skill in the art. See, for example, Maier, M. A., et al. Biodegradable Lipids Enabling Rapidly Eliminated Lipid Nanoparticles for Systemic Delivery of RNAi Therapeutics. Mol. Ther. 2013, 21(8), 1570-78.

Transfer vehicle compositions comprising an amine lipid can lead to an increased clearance rate. In some embodiments, the clearance rate is a lipid clearance rate, for example the rate at which a lipid is cleared from the blood, serum, or plasma. In some embodiments, the clearance rate is an RNA clearance rate, for example the rate at which an circRNA is cleared from the blood, serum, or plasma. In some embodiments, the clearance rate is the rate at which transfer vehicles are cleared from the blood, serum, or plasma. In some embodiments, the clearance rate is the rate at which transfer vehicles are cleared from a tissue, such as liver tissue or spleen tissue. In certain embodiments, a high rate of clearance leads to a safety profile with no substantial adverse effects. The amine lipids and biodegradable lipids may reduce transfer vehicle accumulation in circulation and in tissues. In some embodiments, a reduction in transfer vehicle accumulation in circulation and in tissues leads to a safety profile with no substantial adverse effects.

Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood, where pH is approximately 7.35, the lipid, such as an amine lipid, may not be protonated and thus bear no charge.

The ability of a lipid to bear a charge is related to its intrinsic pKa. In some embodiments, the amine lipids of the present disclosure may each, independently, have a pKa in the range of from about 5.1 to about 7.4. In some embodiments, the bioavailable lipids of the present disclosure may each, independently, have a pKa in the range of from about 5.1 to about 7.4. For example, the amine lipids of the present disclosure may each, independently, have a pKa in the range of from about 5.8 to about 6.5. Lipids with a pKa ranging from about 5.1 to about 7.4 are effective for delivery of cargo in vivo, e.g., to the liver. Further, it has been found that lipids with a pKa ranging from about 5.3 to about 6.4 are effective for delivery in vivo, e.g., into tumors. See, e.g., WO2014/136086.

### 5.2 Lipids Containing a Disulfide Bond

In some embodiments, the ionizable lipid is described in U.S. Pat. No. 9,708,628.

The present invention provides a lipid represented by structure (XXII):

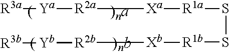

In structure (XXII), X^{a} and X^{b} are each independently X¹ or X² shown below.

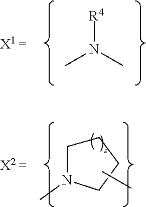

R⁴ in X¹ is an alkyl group having 1-6 carbon atoms, which may be linear, branched or cyclic. The alkyl group preferably has a carbon number of 1-3. Specific examples of the alkyl group having 1-6 carbon atoms include methyl group, ethyl group, propyl group, isopropyl group, n-butyl group, sec-butyl group, isobutyl group, tert-butyl group, pentyl group, isopentyl group, neopentyl group, t-pentyl group, 1,2-dimethylpropyl group, 2-methylbutyl group, 2-methylpentyl group, 3-methylpentyl group, 2,2-dimethylbutyl group, 2,3-dimethylbutyl group, cyclohexyl group and the like. R⁴ is preferably a methyl group, an ethyl group, a propyl group or an isopropyl group, most preferably a methyl group.

The s in X² is 1 or 2. When s is 1, X² is a pyrrolidinium group, and when s is 2, X² is a piperidinium group. s is preferably 2. While the binding direction of X² is not limited, a nitrogen atom in X² preferably binds to R^{1a} and R^{1b}.

X^{a} may be the same as or different from X^{b}, and X^{a} is preferably the same group as X^{b}.

n^{a} and n^{b} are each independently 0 or 1, preferably 1. When n^{a} is 1, R^{3a} binds to X^{a} via Y^{a} and R^{2a}, and when n^{a} is 0, a structure of R^{3a}—X^{a}—R^{1a}—S— is taken. Similarly, when n^{b} is 1, R^{3b} binds to X^{b} via Y^{b} and R^{2b}, and when n^{b} is 0, a structure of R^{3b}—X^{b}R^{1b}—S— is taken.

n^{a} may be the same as or different from n^{b}, and n^{a} is preferably the same as n^{b}.

R^{1a} and R^{1b} are each independently an alkylene group having 1-6 carbon atoms, which may be linear or branched, preferably linear. Specific examples of the alkylene group having 1-6 carbon atoms include methylene group, ethylene group, trimethylene group, isopropylene group, tetramethylene group, isobutylene group, pentamethylene group, neopentylene group and the like. R^{1a} and R^{1b} are each preferably a methylene group, an ethylene group, a trimethylene group, an isopropylene group or a tetramethylene group, most preferably an ethylene group.

R^{1a} may be the same as or different from R^{1b}, and R^{1a} is preferably the same group as R^{1b}.

R^{2a} and R^{2b} are each independently an alkylene group having 1-6 carbon atoms, which may be linear or branched, preferably linear. Examples of the alkylene group having 1-6 carbon atoms include those recited as the examples of the alkylene group having 1-6 carbon atoms for R^{1a} or R^{1b}. R^{2a} and R^{2b} are each preferably a methylene group, an ethylene group, a trimethylene group, an isopropylene group or a tetramethylene group.

When X^{a} and X^{b} are each X¹, R^{2a} and R^{2b} are preferably trimethylene groups. When X^{a} and X^{b} are each X², R^{2a} and R^{2b} are preferably ethylene groups.

R^{2a} may be the same as or different from R^{2b}, and R^{2a} is preferably the same group as R^{2b}.

Y^{a} and Y^{b} are each independently an ester bond, an amide bond, a carbamate bond, an ether bond or a urea bond, preferably an ester bond, an amide bond or a carbamate bond, most preferably an ester bond. While the binding direction of Y^{a} and Y^{b} is not limited, when Y^{a} is an ester bond, a structure of R^{3a}—CO—O—R^{2a}— is preferable, and when Y^{b} is an ester bond, a structure of R^{3b}—CO—O—R^{2b}— is preferable.

Y^{a} may be the same as or different from Y^{b}, and Y^{a} is preferably the same group as Y^{b}.

R^{3a} and R^{3b} are each independently a sterol residue, a liposoluble vitamin residue or an aliphatic hydrocarbon group having 12-22 carbon atoms, preferably a liposoluble vitamin residue or an aliphatic hydrocarbon group having 12-22 carbon atoms, most preferably a liposoluble vitamin residue.

Examples of the sterol residue include a cholesteryl group (cholesterol residue), a cholestaryl group (cholestanol residue), a stigmasteryl group (stigmasterol residue), a β-sitosteryl group (β-sitosterol residue), a lanosteryl group (lanosterol residue), and an ergosteryl group (ergosterol residue) and the like. The sterol residue is preferably a cholesteryl group or a cholestaryl group.

As the liposoluble vitamin residue, a residue derived from liposoluble vitamin, as well as a residue derived from a derivative obtained by appropriately converting a hydroxyl group, aldehyde or carboxylic acid, which is a functional group in liposoluble vitamin, to other reactive functional group can be used. As for liposoluble vitamin having a hydroxyl group, for example, the hydroxyl group can be converted to a carboxylic acid by reacting with succinic acid anhydride, glutaric acid anhydride and the like. Examples of the liposoluble vitamin include retinoic acid, retinol, retinal, ergosterol, 7-dehydrocholesterol, calciferol, cholecalciferol, dihydroergocalciferol, dihydrotachysterol, tocopherol, tocotrienol and the like. Preferable examples of the liposoluble vitamin include retinoic acid and tocopherol.

The aliphatic hydrocarbon group having 12-22 carbon atoms may be linear or branched, preferably linear. The aliphatic hydrocarbon group may be saturated or unsaturated. In the case of an unsaturated aliphatic hydrocarbon group, the aliphatic hydrocarbon group generally contains 1-6, preferably 1-3, more preferably 1-2 unsaturated bonds. While the unsaturated bond includes a carbon-carbon double bond and a carbon-carbon triple bond, it is preferably a carbon-carbon double bond. The aliphatic hydrocarbon group has a carbon number of preferably 12-18, most preferably 13-17. While the aliphatic hydrocarbon group includes an alkyl group, an alkenyl group, an alkynyl group and the like, it is preferably an alkyl group or an alkenyl group. Specific examples of the aliphatic hydrocarbon group having 12-22 carbon atoms include dodecyl group, tridecyl group, tetradecyl group, pentadecyl group, hexadecyl group, heptadecyl group, octadecyl group, nonadecyl group, icosyl group, henicosyl group, docosyl group, dodecenyl group, tridecenyl group, tetradecenyl group, pentadecenyl group, hexadecenyl group, heptadecenyl group, octadecenyl group, nonadecenyl group, icosenyl group, henicosenyl group, docosenyl group, decadienyl group, tridecadienyl group, tetradecadienyl group, pentadecadienyl group, hexadecadienyl group, heptadecadienyl group, octadecadienyl group, nonadecadienyl group, icosadienyl group, henicosadienyl group, docosadienyl group, octadecatrienyl group, icosatrienyl group, icosatetraenyl group, icosapentaenyl group, docosahexaenyl group, isostearyl group and the like. The aliphatic hydrocarbon group having 12-22 carbon atoms is preferably tridecyl group, tetradecyl group, heptadecyl group, octadecyl group, heptadecadienyl group or octadecadienyl group, particularly preferably tridecyl group, heptadecyl group or heptadecadienyl group.

In one embodiment, an aliphatic hydrocarbon group having 12-22 carbon atoms, which is derived from fatty acid, aliphatic alcohol, or aliphatic amine is used. When R^{3a} (or R^{3b}) is derived from fatty acid, Y^{a} (or Y^{b}) is an ester bond or an amide bond, and fatty acid-derived carbonyl carbon is included in Y^{a} (or Y^{b}). For example, when linoleic acid is used, R^{3a} (or R^{3b}) is a heptadecadienyl group.

R^{3a} may be the same as or different from R^{3b}, and R^{3a} is preferably the same group as R^{3b}.

In one embodiment, X^{a} is the same as X^{b}, n^{a} is the same as n^{b}, R^{1a} is the same as R^{1b}, R^{2a} is the same as R^{2b}, R^{3a} is the same as R^{3b}, and Y^{a} is the same as Y^{b}.

In one embodiment,

X^{a} and X^{b} are each independently X1,

R⁴ is an alkyl group having 1-3 carbon atoms, n^{a} and n^{b} are each 1,

R^{1a} and R^{4b} are each independently an alkylene group having 1-6 carbon atoms,

R^{2a} and R^{2b} are each independently an alkylene group having 1-6 carbon atoms,

Y^{a} and Y^{b} are each an ester bond or an amide bond, and

R^{3a} and R^{3b} are each independently an aliphatic hydrocarbon group having 12-22 carbon atoms.

In one embodiment,

X^{a} and X^{b} are each X1,

R⁴ is an alkyl group having 1-3 carbon atoms, n^{a} and n^{b} are each 1,

R^{1a} and R^{1b} are each an alkylene group having 1-6 carbon atoms,

R^{2a} and R^{2b} are each an alkylene group having 1-6 carbon atoms,

Y^{a} and Y^{b} are each an ester bond or an amide bond,

R^{3a} and R^{3b} are each an aliphatic hydrocarbon group having 12-22 carbon atoms,

X^{a} is the same as X^{b},

R^{1a} is the same as R^{1b},

R^{2a} is the same as R^{2b}, and

R^{3a} is the same as R^{3b}.

In one embodiment,

X^{a} and X^{b} are each X¹,

R⁴ is a methyl group, n^{a} and n^{b} are each 1,

R^{1a} and R^{1b} are each an ethylene group,

R^{2a} and R^{2b} are each a trimethylene group,

Y^{a} and Y^{b} are each —CO—O—, and

R^{3a} and R^{3b} are each independently an alkyl group or alkenyl group having 13-17 carbon atoms.

In one embodiment,

X^{a} and X^{b} are each X¹,

R⁴ is a methyl group, n^{a} and n^{b} are each 1,

R^{1a} and R^{1b} are each an ethylene group,

R^{2a} and R^{2b} are each a trimethylene group,

Y^{a} and Y^{b} are each —CO—O—,

R^{3a} and R^{3b} are each an alkyl group or alkenyl group having 13-17 carbon atoms, and

R^{3a} is the same as R^{3b}.

In one embodiment,

X^{a} and X^{b} are each independently X¹,

R⁴ is an alkyl group having 1-3 carbon atoms, n^{a} and n^{b} are each 1,

R^{1a} and R^{1b} are each independently an alkylene group having 1-6 carbon atoms,

R^{2a} and R^{2b} are each independently an alkylene group having 1-6 carbon atoms,

Y^{a} and Y^{b} are each an ester bond or an amide bond, and

R^{3a} and R^{3b} are each independently a liposoluble vitamin residue (e.g., retinoic acid residue, tocopherol residue).

In one embodiment,

X^{a} and X^{b} are each X¹,

R⁴ is an alkyl group having 1-3 carbon atoms, n^{a} and n^{b} are each 1,

R^{1a} and R^{1b} are each an alkylene group having 1-6 carbon atoms,

R^{2a} and R^{2b} are each an alkylene group having 1-6 carbon atoms,

Y^{a} and Y^{b} are each an ester bond or an amide bond,

R^{3a} and R^{3b} are each a liposoluble vitamin residue (e.g., retinoic acid residue, tocopherol residue),

X^{a} is the same as X^{b},

R^{1a} is the same as R^{1b},

R^{2a} is the same as R^{2b}, and

R^{3a} is the same as R^{3b}.

In one embodiment,

X^{a} and X^{b} are each X¹,

R⁴ is a methyl group, n^{a} and n^{b} are each 1,

R^{1a} and R^{1b} are each an ethylene group,

R^{2a} and R^{2b} are each a trimethylene group,

Y^{a} and Y^{b} are each —CO—O—, and

R^{3a} and R^{3b} are each independently a liposoluble vitamin residue (e.g., retinoic acid residue, tocopherol residue).

In one embodiment,

X^{a} and X^{b} are each X¹,

R⁴ is a methyl group, n^{a} and n^{b} are each 1,

R^{1a} and R^{1b} are each an ethylene group,

R^{2a} and R^{2b} are each a trimethylene group,

Y^{a} and Y^{b} are each —CO—O—,

R^{3a} and R^{3b} are each a liposoluble vitamin residue (e.g., retinoic acid residue, tocopherol residue), and

R^{3a} is the same as R^{3b}.

In one embodiment,

X^{a} and X^{b} are each independently X²,

t is 2,

R^{1a} and R^{1b} are each independently an alkylene group having 1-6 carbon atoms,

R^{2a} and R^{2b} are each independently an alkylene group having 1-6 carbon atoms,

Y^{a} and Y^{b} are each an ester bond, and

R^{3a} and R^{3b} are each independently a liposoluble vitamin residue (e.g., retinoic acid residue, tocopherol residue) or an aliphatic hydrocarbon group having 12-22 carbon atoms (e.g., alkyl group having 12-22 carbon atoms).

In one embodiment,

X^{a} and X^{b} are each independently X²,

t is 2,

R^{1a} and R^{1b} are each independently an alkylene group having 1-6 carbon atoms,

R^{2a} and R^{2b} are each independently an alkylene group having 1-6 carbon atoms,

Y^{a} and Y^{b} are each an ester bond,

R^{3a} and R^{3b} are each independently a liposoluble vitamin residue (e.g., retinoic acid residue, tocopherol residue) or an aliphatic hydrocarbon group having 12-22 carbon atoms (e.g., alkyl group having 12-22 carbon atoms),

X^{a} is the same as X^{b},

R^{1a} is the same as R^{1b},

R^{2a} is the same as R^{2b}, and

R^{3a} is the same as R^{3b}.

In one embodiment,

X^{a} and X^{b} are each independently X²,

t is 2,

R^{1a} and R^{1b} are each an ethylene group,

R^{2a} and R^{2b} are each independently an alkylene group having 1-6 carbon atoms,

Y^{a} and Y^{b} are each an ester bond,

R^{3a} and R^{3b} are each independently a liposoluble vitamin residue (e.g., retinoic acid residue, tocopherol residue) or an aliphatic hydrocarbon group having 12-22 carbon atoms (e.g., alkyl group having 12-22 carbon atoms),

X^{a} is the same as X^{b},

R^{2a} is the same as R^{2b}, and

R^{3a} is the same as R^{3b}.

In some embodiments, an ionizable lipid has one of the structures set forth in Table 15b below.

TABLE 15b

| Number | Structure |
|---|---|
| 1 | 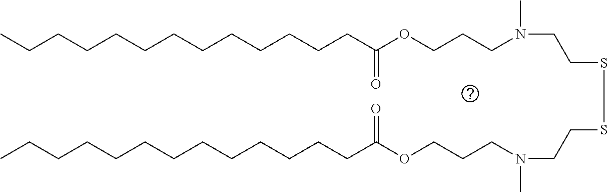 [SMILES: CCCCCCCCCCCCCC(=O)OCCCN(C)CCSCSCCN(C)CCCOC(=O)CCCCCCCCCCCCC] |
| 2 | 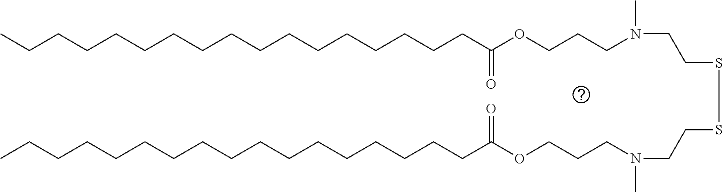 [SMILES: CCCCCCCCCCCCCCCCCC(=O)OCCCN(C)CCSCSCCN(C)CCCOC(=O)CCCCCCCCCCCCCCCCC] |
| 3 | 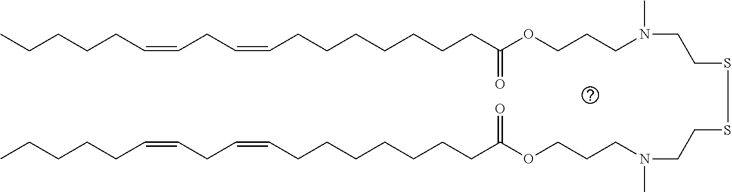 [SMILES: CCCCC/C=C\C/C=C\CCCCCCCC(=O)OCCCN(C)CCSCSCCN(C)CCCOC(=O)CCCCCCC/C=C\C/C=C\CCCCC] |
| 4 | 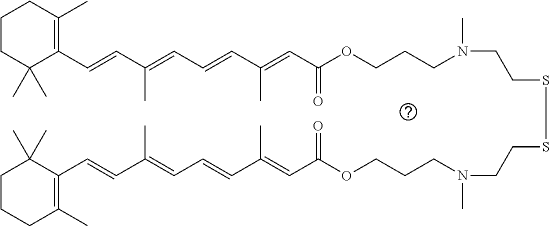 [SMILES: CC1=C(/C=C/C(C)=C/C=C/C(C)=C/C(=O)OCCCN(C)CCSCSCCN(C)CCCOC(=O)/C=C(C)/C=C/C=C(C)/C=C/C2=C(C)CCCC2(C)C)C(C)(C)CCC1] |
| 5 | 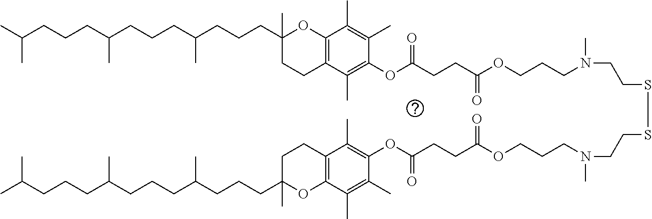 [SMILES: Cc1c(C)c2c(c(C)c1OC(=O)CCC(=O)OCCCN(C)CCSCSCCN(C)CCCOC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2] |
| 6 | 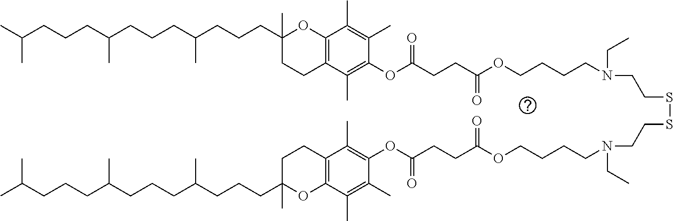 [SMILES: CCN(CCCCOC(=O)CCC(=O)Oc1c(C)c(C)c2c(c1C)CCC(C)(CCCC(C)CCCC(C)CCCC(C)C)O2)CCSSCCN(CC)CCCCOC(=O)CCC(=O)Oc1c(C)c(C)c2c(c1C)CCC(C)(CCCC(C)CCCC(C)CCCC(C)C)O2] |
| 7 | 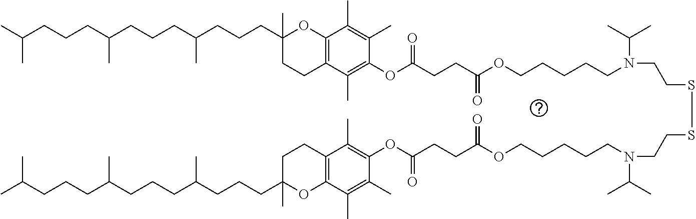 [SMILES: Cc1c(C)c2c(c(C)c1OC(=O)CCC(=O)OCCCCCN(CCSCSCCN(CCCCCOC(=O)CCC(=O)Oc1c(C)c(C)c3c(c1C)CCC(C)(CCCC(C)CCCC(C)CCCC(C)C)O3)C(C)C)C(C)C)CCC(C)(CCCC(C)CCCC(C)CCCC(C)C)O2] |
| 8 | 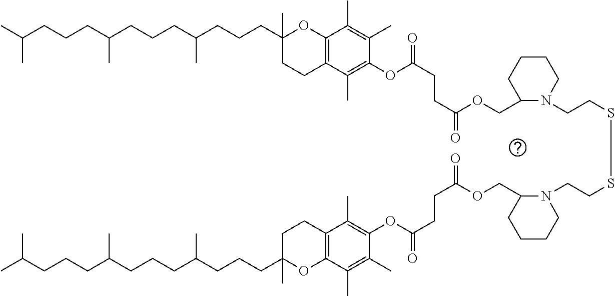 [SMILES: Cc1c(C)c2c(c(C)c1OC(=O)CCC(=O)OCC1CCCCN1CCSCSCCN1CCCCC1COC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2] |
| 9 | 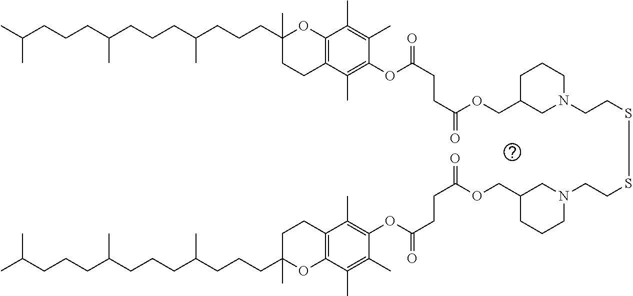 [SMILES: Cc1c(C)c2c(c(C)c1OC(=O)CCC(=O)OCC1CCCN(CCSCSCCN3CCCC(COC(=O)CCC(=O)Oc4c(C)c(C)c5c(c4C)CCC(C)(CCCC(C)CCCC(C)CCCC(C)C)O5)C3)C1)CCC(C)(CCCC(C)CCCC(C)CCCC(C)C)O2] |
| 10 | 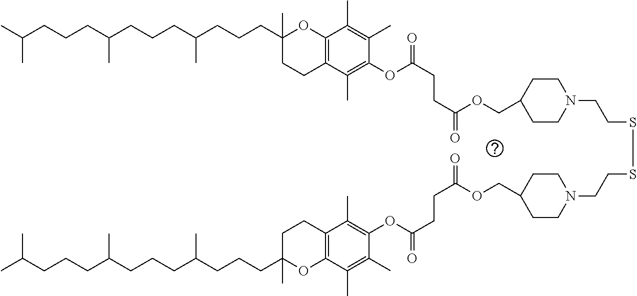 [SMILES: Cc1c(C)c2c(c(C)c1OC(=O)CCC(=O)OCC1CCN(CCSCSCCN3CCC(COC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2] |
| 11 | 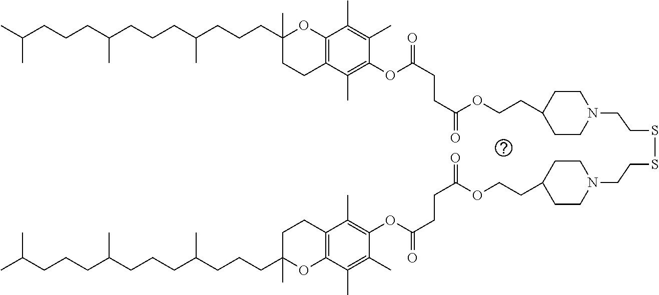 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2] |
| 12 | 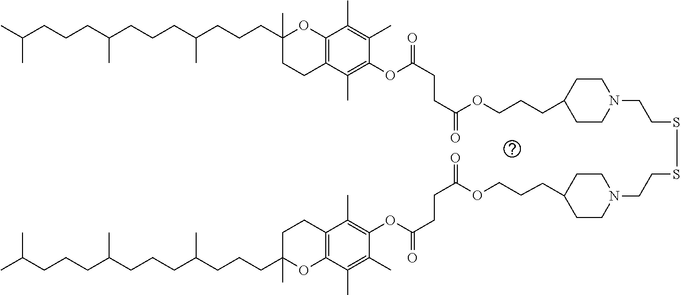 [SMILES: Cc1c(C)c2c(c(C)c1OC(=O)CCC(=O)OCCCC1CCN(CCSSCCN3CCC(CCCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2] |
| 13 | 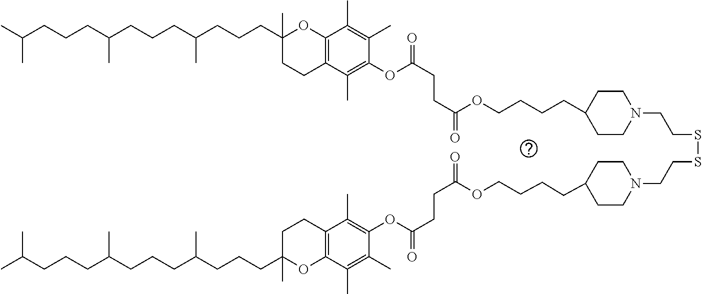 [SMILES: Cc1c(C)c2c(c(C)c1OC(=O)CCC(=O)OCCCCC1CCN(CCSSCCN3CCC(CCCCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2] |
| 14 | 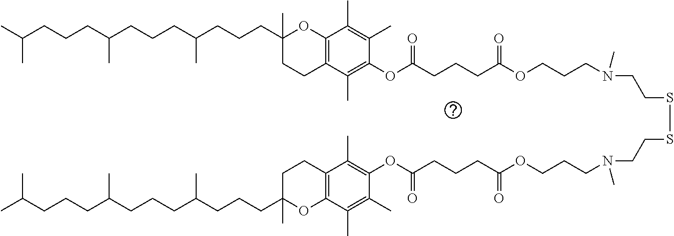 [SMILES: Cc1c(C)c2c(c(C)c1OC(=O)CCCC(=O)OCCCN(C)CCSSCCN(C)CCCOC(=O)CCCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2] |
| 15 | 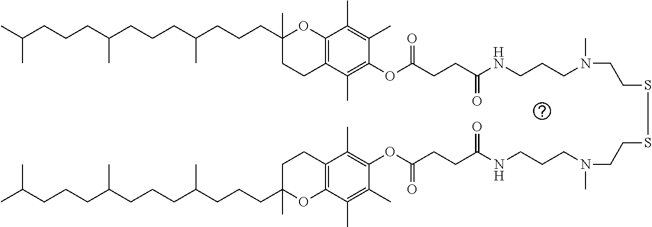 [SMILES: Cc1c(C)c2c(c(C)c1OC(=O)CCC(=O)NCCCN(C)CCSSCCN(C)CCCNC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2] |

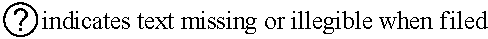

A lipid of the present invention may have an —S—S— (disulfide) bond. The production method for such a compound includes, for example, a method including producing

R^{3a}—(Y^{a}—R^{2a})n^{a}-X^{a}—R^{1a}—SH, and

R^{3b}—(Y^{b}—R^{2b})n^{b}-X^{b}—R^{1b}—SH, and

subjecting them to oxidation (coupling) to give a compound containing —S—S—, a method including sequentially bonding necessary parts to a compound containing an —S—S— bond to finally obtain the compound of the present invention and the like. Preferred is the latter method.

A specific example of the latter method is shown below, which is not to be construed as limiting.

Examples of the starting compound include —S—S— bond-containing two terminal carboxylic acid, two terminal carboxylate, two terminal amine, two terminal isocyanate, two terminal alcohol, two terminal alcohol having a leaving group such as MsO (mesylate group) and the like, a two terminal carbonate having a leaving group such as pNP (p-nitrophenylcarbonate group) and the like.

For example, when a compound containing X¹ or X² for X^{a}and X^{b} is produced, two terminal functional groups of compound (1) containing an —S—S— bond are reacted with an —NH— group in compound (2) having the —NH— group and one functional group at the terminal, the functional group at the terminal in compound (2) which did not contribute to the reaction is reacted with a functional group in compound (3) containing R³, whereby the compound of the present invention containing an —S—S— bond, R^{1a} and R^{1b}, X^{a} and X^{b}, R^{2a} and R^{2b}, Y^{a} and Y^{b}, and R^{3a} and R^{3b} can be obtained.

In the reaction of compound (1) and compound (2), an alkali catalyst such as potassium carbonate, sodium carbonate, potassium t-butoxide and the like may be used as a catalyst, or the reaction may be performed without a catalyst. Preferably, potassium carbonate or sodium carbonate is used as a catalyst.

The amount of catalyst is 0.1-100 molar equivalents, preferably, 0.1-20 molar equivalents, more preferably 0.1-5 molar equivalents, relative to compound (1). The amount of compound (2) to be charged is 1-50 molar equivalents, preferably 1-10 molar equivalents, relative to compound (1).

The solvent to be used for the reaction of compound (1) and compound (2) is not particularly limited as long as it is a solvent or aqueous solution that does not inhibit the reaction. For example, ethyl acetate, dichloromethane, chloroform, benzene, toluene and the like can be mentioned. Among these, toluene and chloroform are preferable.

The reaction temperature is −20 to 200° C., preferably 0 to 80° C., more preferably 20 to 50° C., and the reaction time is 1-48 hr, preferably 2-24 hr.

When the reaction product of compound (1) and compound (2) is reacted with compound (3), an alkali catalyst such as potassium carbonate, sodium carbonate, potassium t-butoxide and the like, or an acid catalyst such as PTS (p-toluenesulfonic acid), MSA (methanesulfonic acid) and the like may be used, like the catalyst used for the reaction of compound (1) and compound (2), or the reaction may be performed without a catalyst.

In addition, the reaction product of compound (1) and compound (2) may be directly reacted with compound (3) by using a condensing agent such as DCC (dicyclohexylcarbodiimide), DIC (diisopropylcarbodiimide), EDC (1-ethyl-3-(3-dimethylaminopropyl)carbodiimide hydrochloride) and the like. Alternatively, compound (3) may be treated with a condensing agent to be once converted to an anhydride and the like, after which it is reacted with the reaction product of compound (1) and compound (2).

The amount of compound (3) to be charged is 1-50 molar equivalents, preferably 1-10 molar equivalents, relative to the reaction product of compound (1) and compound (2).

The catalyst to be used is appropriately selected according to the functional groups to be reacted.

The amount of catalyst is 0.05-100 molar equivalents, preferably 0.1-20 molar equivalents, more preferably 0.2-5 molar equivalent, relative to compound (1).

The solvent to be used for the reaction of the reaction product of compound (1) and compound (2) with compound (3) is not particularly limited as long as it is a solvent or aqueous solution that does not inhibit the reaction. For example, ethyl acetate, dichloromethane, chloroform, benzene, toluene and the like can be mentioned. Among these, toluene and chloroform are preferable.

The reaction temperature is 0 to 200° C., preferably 0 to 120° C., more preferably 20 to 50° C., and the reaction time is 1 hr-48 hr, preferably 2-24 hr.

The reaction product obtained by the above-mentioned reaction can be appropriately purified by a general purification method, for example, washing with water, silica gel column chromatography, crystallization, recrystallization, liquid-liquid extraction, reprecipitation, ion exchange column chromatography and the like.

### 5.3 Structure XXIII Lipids

In some embodiments, an ionizable lipid is described in U.S. Pat. No. 9,765,022.

The present invention provides a compound represented by structure (XXIII):

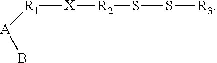

In structure XXIII, a hydrophilic and optionally positively charged head is

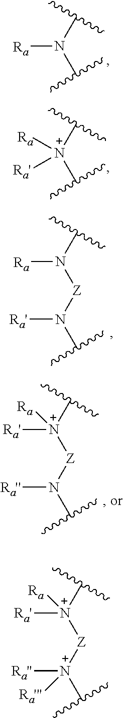

in which each of Rₐ′, Rₐ″, and Rₐ′″, independently, is H, a C₁-C₂₀ monovalent aliphatic radical, a C₁-C₂₀ monovalent heteroaliphatic radical, a monovalent aryl radical, or a monovalent heteroaryl radical, and Z is a C₁-C₂₀ bivalent aliphatic radical, a C₁-C₂₀ bivalent heteroaliphatic radical, a bivalent aryl radical, or a bivalent heteroaryl radical; B is a C₁-C₂₄ monovalent aliphatic radical, a C₁-C₂₄ monovalent heteroaliphatic radical, a monovalent aryl radical, a monovalent heteroaryl radical, or

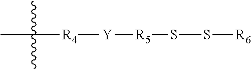

each of R₁ and R₄, independently, is a bond, a C₁-C₁₀ bivalent aliphatic radical, a C₁-C₁₀ bivalent heteroaliphatic radical, a bivalent aryl radical, or a bivalent heteroaryl radical; each of R₂ and R₅, independently, is a bond, a C₁-C₂₀ bivalent aliphatic radical, a C₁-C₂₀ bivalent heteroaliphatic radical, a bivalent aryl radical, or a bivalent heteroaryl radical; each of R₃ and R₆, independently, is a C₁-C₂₀ monovalent aliphatic radical, a C₁-C₂₀ monovalent heteroaliphatic radical, a monovalent aryl radical, or a monovalent heteroaryl radical; each of

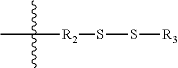

a hydrophobic tail, and

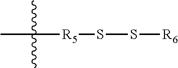

also a hydrophobic tail, has 8 to 24 carbon atoms; and each of X, a linker, and Y, also a linker, independently, is

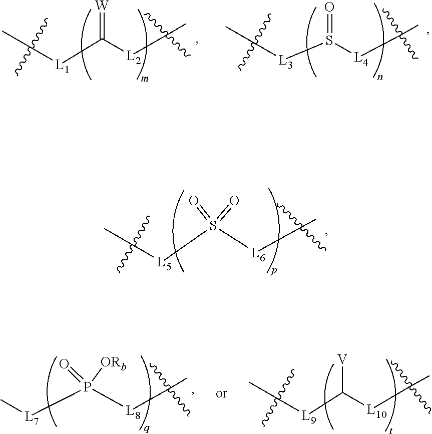

in which each of m, n, p, q, and t, independently, is 1-6; W is O, S, or NR_{c}; each of L₁, L₃, L₅, L₇, and L₉, directly linked to R₁, R₂, R₄, or R₅, independently, is a bond, O, S, or NR_{d}; each of L₂, L₄, L₆, L₈, and L₁₀, independently, is a bond, O, S, or NRₑ; V is OR_{f}, SR_{g}, or NRₕRᵢ; and each of R_{b}, R_{c}, R_{d}, Rₑ, R_{f}, R_{g}, Rₕ, and Rᵢ, independently, is H, OH, C₁₋₁₀ oxyaliphatic radical, C₁-C₁₀ monovalent aliphatic radical, C₁-C₁₀ monovalent heteroaliphatic radical, a monovalent aryl radical, or a monovalent heteroaryl radical.

A subset of the above-described lipid-like compounds include those in which A is

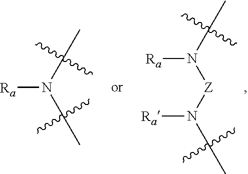

each of Rₐ and Rₐ′, independently, being a C₁-C₁₀ monovalent monovalent aliphatic radical, a C₁-C₁₀ monovalent heteroaliphatic radical, a monovalent aryl radical, or a monovalent heteroaryl radical; and Z being a C₁-C₁₀ bivalent aliphatic radical, a C₁-C₁₀ bivalent heteroaliphatic radical, a bivalent aryl radical, or a bivalent heteroaryl radical.

Some lipid-like compounds of this invention feature each of R₁ and R₄, independently, being C₁-C₆ (e.g., C₁-C₄) bivalent aliphatic radical or a C₁-C₆ (e.g., C₁-C₄) bivalent heteroaliphatic radical, the total carbon number for R₂ and R₃ being 12-20 (e.g., 14-18), the total carbon number of R₅ and R₆ also being 12-20 (e.g., 14-18), and each of X and Y, independently, is

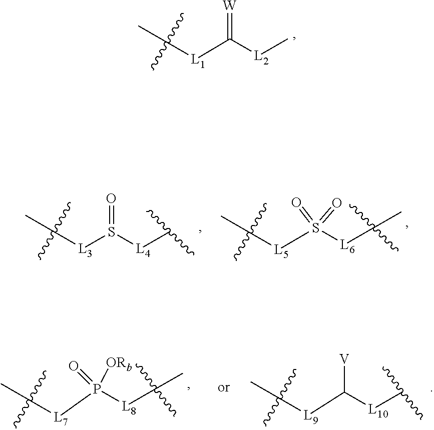

Specific examples of X and Y include

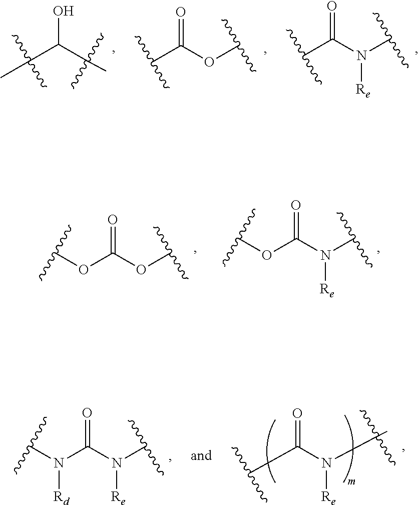

m being 2-6.

Still within the scope of this invention is a pharmaceutical composition containing a nanocomplex that is formed of a protein and a bioreducible compound. In this pharmaceutical composition, the nanocomplex has a particle size of 50 to 500 nm; the bioreducible compound contains a disulfide hydrophobic moiety, a hydrophilic moiety, and a linker joining the disulfide hydrophobic moiety and the hydrophilic moiety; and the protein binds to the bioreducible compound via a non-covalent interaction, a covalent bond, or both.

In certain embodiments, the disulfide hydrophobic moiety is a heteroaliphatic radical containing one or more —S—S— groups and 8 to 24 carbon atoms; the hydrophilic moiety is an aliphatic or heteroaliphatic radical containing one or more hydrophilic groups and 1-20 carbon atoms, each of the hydrophilic groups being amino, alkylamino, dialkylamino, trialkylamino, tetraalkylammonium, hydroxyamino, hydroxyl, carboxyl, carboxylate, carbamate, carbamide, carbonate, phosphate, phosphite, sulfate, sulfite, or thiosulfate; and the linker is O, S, Si, C₁-C₆ alkylene,

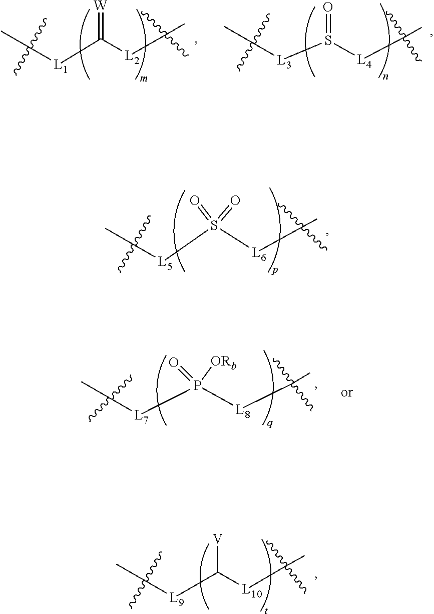

in which the variables are defined above.

Specific examples of X and Y include O, S, Si, C₁-C₆ alkylene,

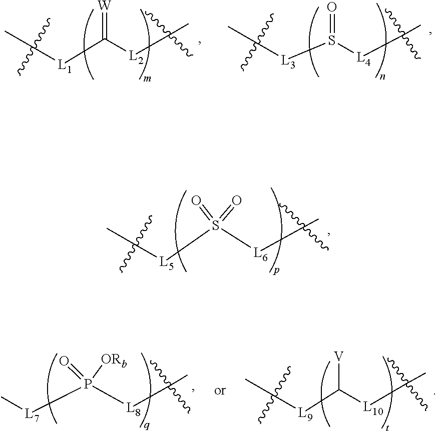

In some embodiments, a lipid-like compound of this invention, as shown in structure XXIII above, includes (i) a hydrophilic head, A; (ii) a hydrophobic tail, R₂—S—S—R₃; and (iii) a linker, X. Optionally, these compounds contain a second hydrophobic tail, R₅—S—S—R₆ and a second linker, Y.

The hydrophilic head of structure XXIII contains one or more hydrophilic functional groups, e.g., hydroxyl, carbonyl, carboxyl, amino, sulfhydryl, phosphate, amide, ester, ether, carbamate, carbonate, carbamide, and phosphodiester. These groups can form hydrogen bonds and are optionally positively or negatively charged.

Examples of the hydrophilic head include:

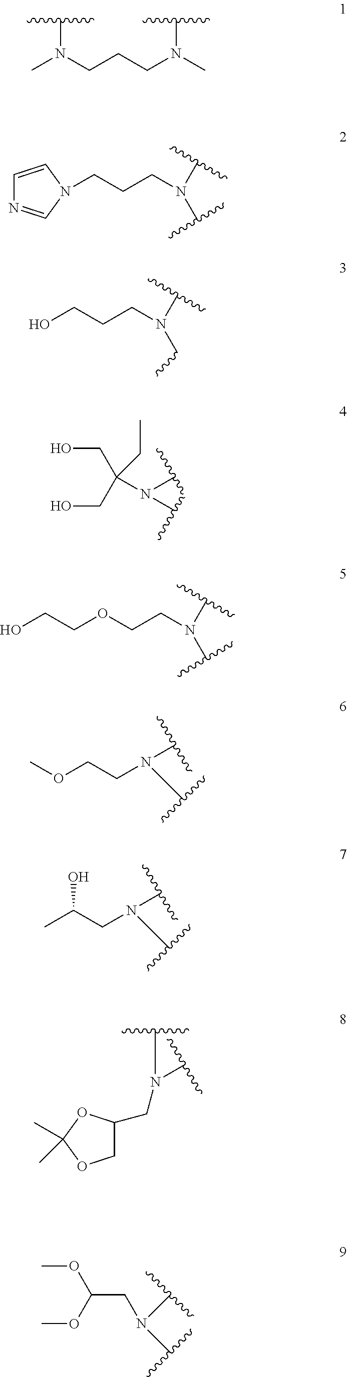

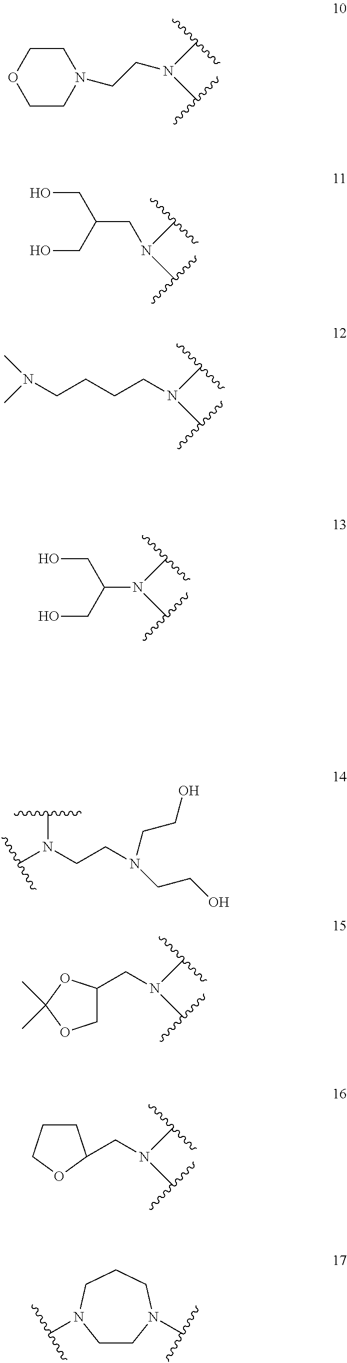

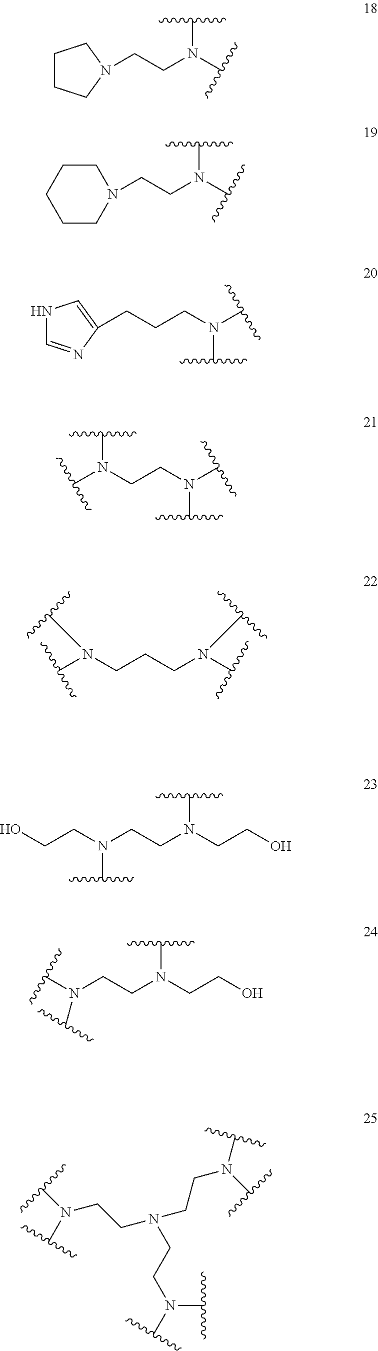

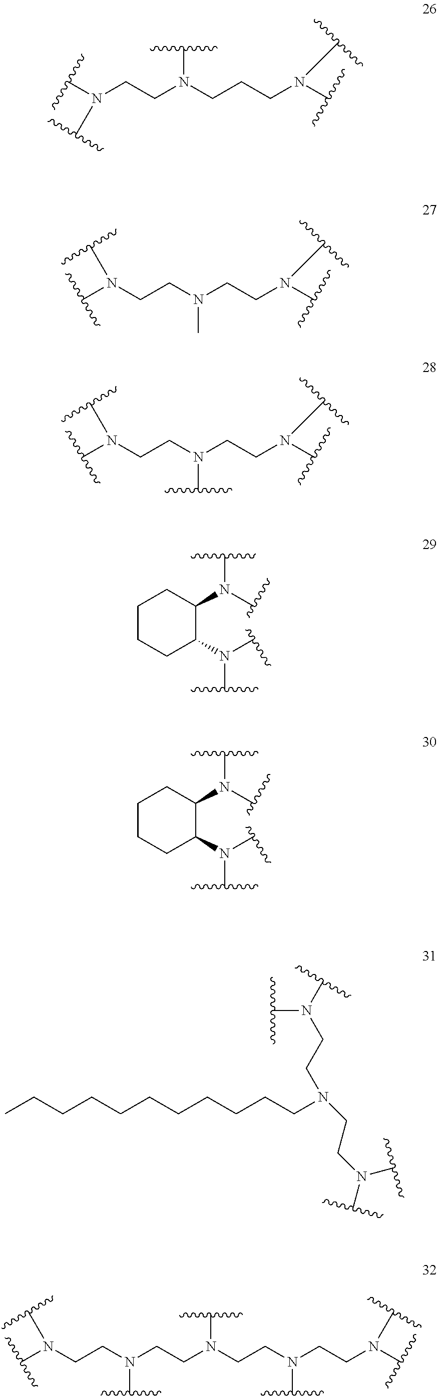

Other examples include those described in Akinc et al., Nature Biotechnology, 26, 561-69 (2008) and Mahon et al., US Patent Application Publication 2011/0293703.

The hydrophobic tail of structure XXIII is a saturated or unsaturated, linear or branched, acyclic or cyclic, aromatic or nonaromatic hydrocarbon moiety containing a disulfide bond and 8-24 carbon atoms. One or more of the carbon atoms can be replaced with a heteroatom, such as N, O, P, B, S, Si, Sb, Al, Sn, As, Se, and Ge. The tail is optionally substituted with one or more groups described above. The lipid-like compounds containing this disulfide bond can be bioreducible.

Examples include:

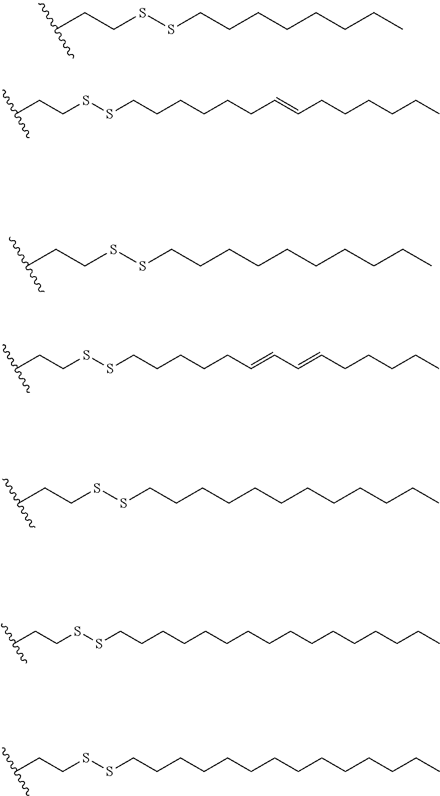

A linker of structure XXIII links the hydrophilic head and the hydrophobic tail. The linker can be any chemical group that is hydrophilic or hydrophobic, polar or non-polar, e.g., O, S, Si, amino, alkylene, ester, amide, carbamate, carbamide, carbonate, phosphate, phosphite, sulfate, sulfite, and thiosulfate. Examples include:

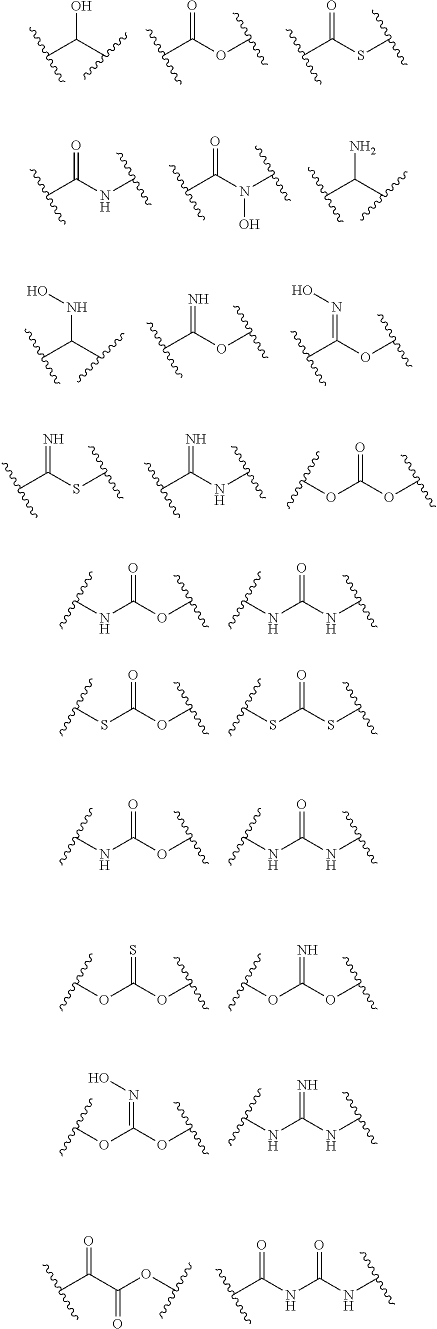

Shown below are exemplary lipid-like compounds of this invention:

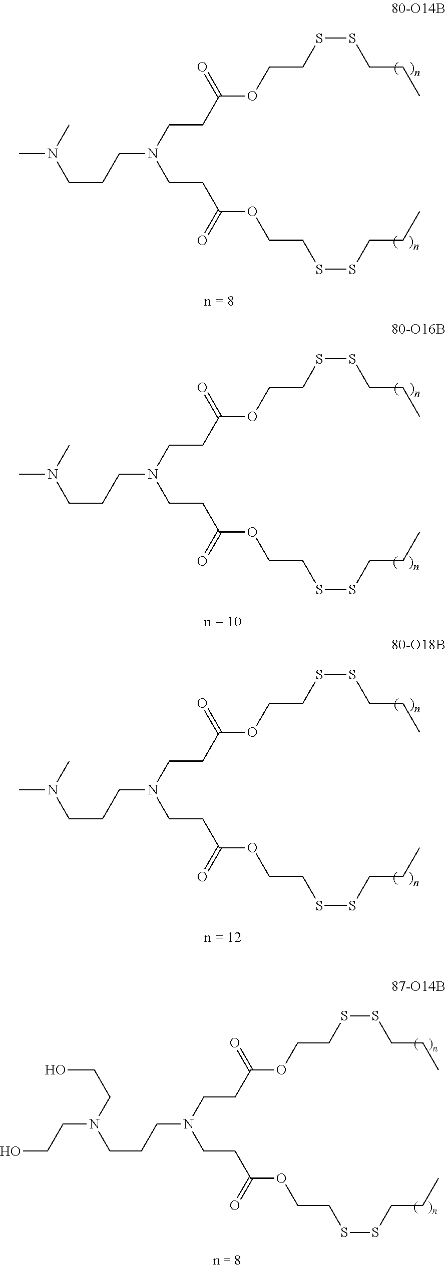

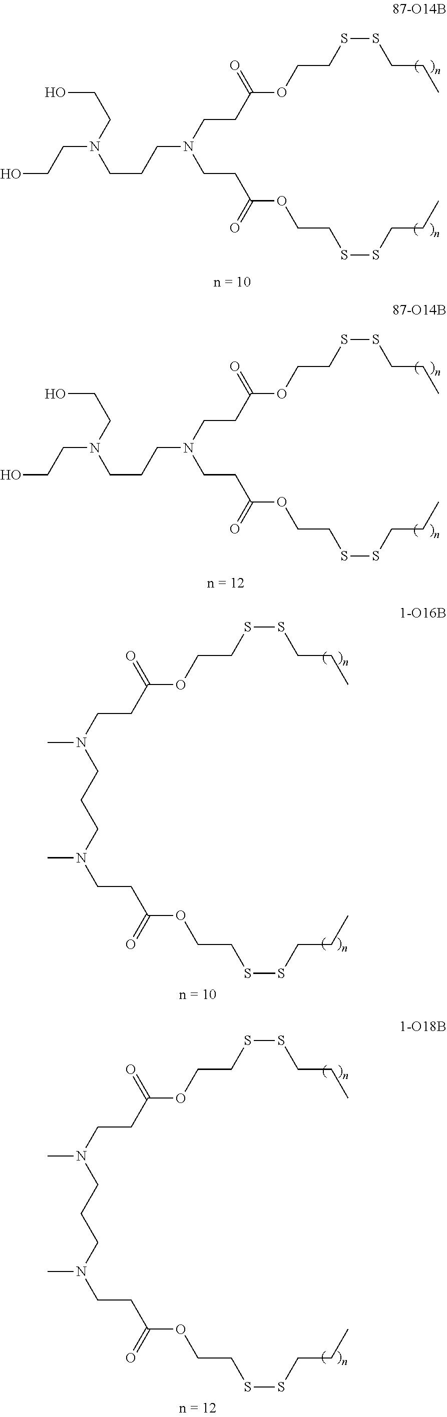

The lipid-like compounds of structure XXIII can be prepared by methods well known the art. See Wang et al., ACS Synthetic Biology, 1, 403-07 (2012); Manoharan, et al., International Patent Application Publication WO 2008/042973; and Zugates et al., U.S. Pat. No. 8,071,082. The route shown below exemplifies synthesis of these lipid-like compounds:

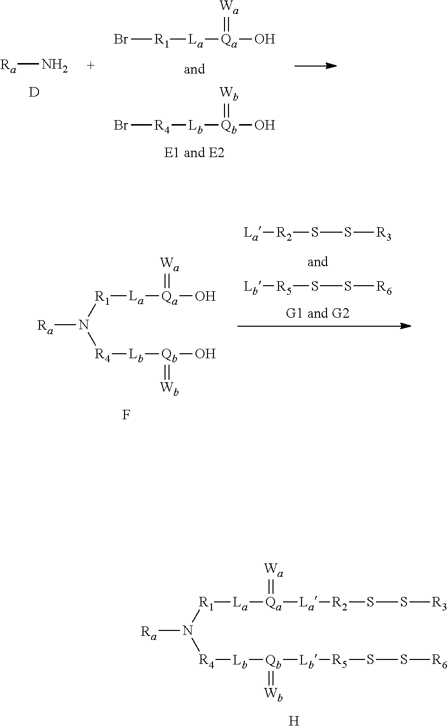

Each of Lₐ, Lₐ′, L, and L′ can be one of L₁-L₁₀; each of Wₐ and W_{b}, independently, is W or V; and Rₐ and R₁-R₆ are defined above, as well as L₁-L₁₀, W, and V.

In this exemplary synthetic route, an amine compound, i.e., compound D, reacts with bromides E1 and E2 to form compound F, which is then coupled with both G1 and G2 to afford the final product, i.e., compound H. One or both of the double bonds in this compound (shown above) can be reduced to one or two single bonds to obtain different lipid-like compounds of structure XXIII.

Other lipid-like compounds of this invention can be prepared using other suitable starting materials through the above-described synthetic route and others known in the art. The method set forth above can include an additional step(s) to add or remove suitable protecting groups in order to ultimately allow synthesis of the lipid-like compounds. In addition, various synthetic steps can be performed in an alternate sequence or order to give the desired material. Synthetic chemistry transformations and protecting group methodologies (protection and deprotection) useful in synthesizing applicable lipid-like compounds are known in the art, including, for example, R. Larock, Comprehensive Organic Transformations (2nd Ed., VCH Publishers 1999); P. G. M. Wuts and T. W. Greene, Greene's Protective Groups in Organic Synthesis (4th Ed., John Wiley and Sons 2007); L. Fieser and M. Fieser, Fieser and Fieser's Reagents for Organic Synthesis (John Wiley and Sons 1994); and L. Paquette, ed., Encyclopedia of Reagents for Organic Synthesis (2nd ed., John Wiley and Sons 2009) and subsequent editions thereof. Certain lipid-like compounds may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

As mentioned above, these lipid-like compounds are useful for delivery of pharmaceutical agents. They can be preliminarily screened for their efficacy in delivering pharmaceutical agents by an in vitro assay and then confirmed by animal experiments and clinic trials. Other methods will also be apparent to those of ordinary skill in the art.

Not to be bound by any theory, the lipid-like compounds of structure XXIII facilitate delivery of pharmaceutical agents by forming complexes, e.g., nanocomplexes and microparticles. The hydrophilic head of such a lipid-like compound, positively or negatively charged, binds to a moiety of a pharmaceutical agent that is oppositely charged and its hydrophobic moiety binds to a hydrophobic moiety of the pharmaceutical agent. Either binding can be covalent or non-covalent.

The above described complexes can be prepared using procedures described in publications such as Wang et al., ACS Synthetic Biology, 1, 403-07 (2012). Generally, they are obtained by incubating a lipid-like compound and a pharmaceutical agent in a buffer such as a sodium acetate buffer or a phosphate buffered saline (“PBS”).

### 5.4 Hydrophilic Groups

In certain embodiments, the selected hydrophilic functional group or moiety may alter or otherwise impart properties to the compound or to the transfer vehicle of which such compound is a component (e.g., by improving the transfection efficiencies of a lipid nanoparticle of which the compound is a component). For example, the incorporation of guanidinium as a hydrophilic head-group in the compounds disclosed herein may promote the fusogenicity of such compounds (or of the transfer vehicle of which such compounds are a component) with the cell membrane of one or more target cells, thereby enhancing, for example, the transfection efficiencies of such compounds. It has been hypothesized that the nitrogen from the hydrophilic guanidinium moiety forms a six-membered ring transition state which grants stability to the interaction and thus allows for cellular uptake of encapsulated materials. (Wender, et al., Adv. Drug Del. Rev. (2008) 60: 452-472.) Similarly, the incorporation of one or more amino groups or moieties into the disclosed compounds (e.g., as a head-group) may further promote disruption of the endosomal/lysosomal membrane of the target cell by exploiting the fusogenicity of such amino groups. This is based not only on the pKa of the amino group of the composition, but also on the ability of the amino group to undergo a hexagonal phase transition and fuse with the target cell surface, i.e. the vesicle membrane. (Koltover, et al. Science (1998) 281: 78-81.) The result is believed to promote the disruption of the vesicle membrane and release of the lipid nanoparticle contents into the target cell.

Similarly, in certain embodiments the incorporation of, for example, imidazole as a hydrophilic head-group in the compounds disclosed herein may serve to promote endosomal or lysosomal release of, for example, contents that are encapsulated in a transfer vehicle (e.g., lipid nanoparticle) of the invention. Such enhanced release may be achieved by one or both of a proton-sponge mediated disruption mechanism and/or an enhanced fusogenicity mechanism. The proton-sponge mechanism is based on the ability of a compound, and in particular a functional moiety or group of the compound, to buffer the acidification of the endosome. This may be manipulated or otherwise controlled by the pKa of the compound or of one or more of the functional groups comprising such compound (e.g., imidazole). Accordingly, in certain embodiments the fusogenicity of, for example, the imidazole-based compounds disclosed herein (e.g., HGT4001 and HGT4004) are related to the endosomal disruption properties, which are facilitated by such imidazole groups, which have a lower pKa relative to other traditional ionizable lipids. Such endosomal disruption properties in turn promote osmotic swelling and the disruption of the liposomal membrane, followed by the transfection or intracellular release of the polynucleotide materials loaded or encapsulated therein into the target cell. This phenomenon can be applicable to a variety of compounds with desirable pKa profiles in addition to an imidazole moiety. Such embodiments also include multi-nitrogen based functionalities such as polyamines, polypeptide (histidine), and nitrogen-based dendritic structures.

Exemplary ionizable and/or cationic lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004 143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety. International patent application WO 2019/131770 is also incorporated herein by reference in its entirety.

### 1. PEG Lipids

The use and inclusion of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) in the liposomal and pharmaceutical compositions described herein is contemplated, preferably in combination with one or more of the compounds and lipids disclosed herein. Contemplated PEG-modified lipids include, but are not limited to, a polyethylene glycol chain of up to 5 kDa in length covalently attached to a lipid with alkyl chain(s) of C6-C20 length. In some embodiments, the PEG-modified lipid employed in the compositions and methods of the invention is 1,2-dimyristoyl-sn-glycerol, methoxypolyethylene Glycol (2000 MW PEG) “DMG-PEG2000.” The addition of PEG-modified lipids to the lipid delivery vehicle may prevent complex aggregation and may also provide a means for increasing circulation lifetime and increasing the delivery of the lipid-polynucleotide composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivatized lipids of the present invention may comprise a molar ratio from about 0% to about 20%, about 0.5% to about 20%, about 1% to about 15%, about 4% to about 10%, or about 2% of the total lipid present in a liposomal lipid nanoparticle.

In an embodiment, a PEG-modified lipid is described in International Pat. Appl. No. PCT/US2019/015913, which is incorporated herein by reference in their entirety. In an embodiment, a transfer vehicle comprises one or more PEG-modified lipids.

Non-limiting examples of PEG-modified lipids include PEG-modified phosphatidylethanolamines and phosphatidic acids, PEG-ceramide conjugates (e.g., PEG-CerC14 or PEG-CerC20), PEG-modified dialkylamines and PEG-modified 1,2-diacyloxypropan-3-amines. In some further embodiments, a PEG-modified lipid may be, e.g., PEG-c-DOMG, PEG-DMG, PEG-DLPE, PEG-DMPE, PEG-DPPC, or a PEG-DSPE.

In some still further embodiments, the PEG-modified lipid includes, but is not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In various embodiments, a PEG-modified lipid may also be referred to as “PEGylated lipid” or “PEG-lipid.”

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, such as from about C₁₄ to about C₁₆. In some embodiments, a PEG moiety, for example a mPEG-NH₂, has a size of about 1000, about 2000, about 5000, about 10,000, about 15,000 or about 20,000 daltons. In one embodiment, the PEG-lipid is PEG2k-DMG.

In one embodiment, the lipid nanoparticles described herein can comprise a lipid modified with a non-diffusible PEG. Non-limiting examples of non-diffusible PEGs include PEG-DSG and PEG-DSPE.

PEG-lipids are known in the art, such as those described in U.S. Pat. No. 8,158,601 and International Pat. Publ. No. WO2015/130584 A2, which are incorporated herein by reference in their entirety.

In various embodiments, lipids (e.g., PEG-lipids), described herein may be synthesized as described International Pat. Publ. No. PCT/US2016/000129, which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG-DMG. PEG-DMG has the following structure:

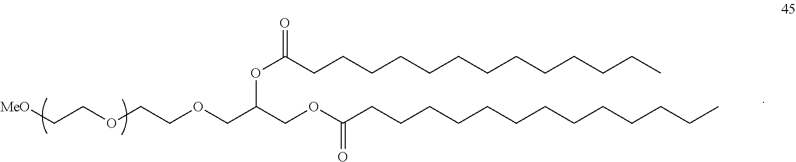

In some embodiments the PEG-modified lipids are a modified form of PEG-C18, or PEG-1. PEG-1 has the following structure

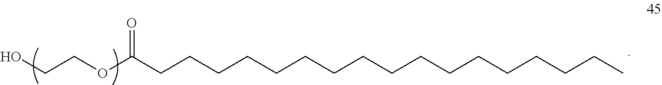

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In some embodiments, the PEG lipid is a compound of Formula (P1):

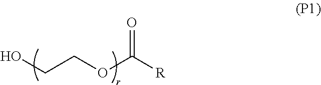

or a salt or isomer thereof, wherein:
r is an integer between 1 and 100;
R is C₁₀₋₄₀ alkyl, C₁₀₋₄₀ alkenyl, or C₁₀₋₄₀ alkynyl; and optionally one or more methylene groups of R are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and
each instance of R^{N} is independently hydrogen, C₁₋₆ alkyl, or a nitrogen protecting group.

For example, R is C17 alkyl. For example, the PEG lipid is a compound of Formula (P1-a):

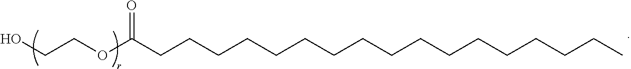

or a salt or isomer thereof, wherein r is an integer between 1 and 100.

For example, the PEG lipid is a compound of the following formula:

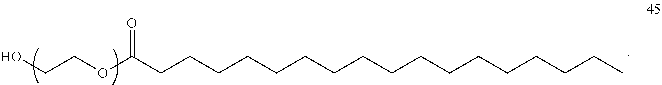

### 2. Helper Lipids

In some embodiments, the transfer vehicle (e.g., LNP) described herein comprises one or more non-cationic helper lipids. In some embodiments, the helper lipid is a phospholipid. In some embodiments, the helper lipid is a phospholipid substitute or replacement. In some embodiments, the phospholipid or phospholipid substitute can be, for example, one or more saturated or (poly)unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidyl glycerols, and phosphatidic acids. Phospholipids also include phosphosphingolipid, such as sphingomyelin.

In some embodiments, the helper lipid is a 1,2-distearoyl-177-glycero-3-phosphocholine (DSPC) analog, a DSPC substitute, oleic acid, or an oleic acid analog.

In some embodiments, a helper lipid is a non-phosphatidyl choline (PC) zwitterionic lipid, a DSPC analog, oleic acid, an oleic acid analog, or a DSPC substitute.

In some embodiments, a helper lipid is described in PCT/US2018/053569. Helper lipids suitable for use in a lipid composition of the disclosure include, for example, a variety of neutral, uncharged or zwitterionic lipids. Such helper lipids are preferably used in combination with one or more of the compounds and lipids disclosed herein. Examples of helper lipids include, but are not limited to, 5-heptadecylbenzene-1,3-diol (resorcinol), dipalmitoylphosphatidylcholine (DPPC), di stearoylphosphatidylcholine (DSPC), pohsphocholine (DOPC), dimyristoylphosphatidylcholine (DMPC), phosphatidylcholine (PLPC), 1,2-distearoylsn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-paimitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), paimitoyioieoyl phosphatidylcholine (POPC), lysophosphatidyl choline, dioleoyl phosphatidylethanol amine (DOPE) dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the helper lipid may be distearoylphosphatidylcholine (DSPC) or dimyristoyl phosphatidyl ethanolamine (DMPE). In another embodiment, the helper lipid may be distearoylphosphatidylcholine (DSPC). Helper lipids function to stabilize and improve processing of the transfer vehicles. Such helper lipids are preferably used in combination with other excipients, for example, one or more of the ionizable lipids disclosed herein. In some embodiments, when used in combination with an ionizable lipid, the helper lipid may comprise a molar ratio of 5% to about 90%, or about 10% to about 70% of the total lipid present in the lipid nanoparticle.

### 3. Structural Lipids

In an embodiment, a structural lipid is described in international patent application PCT/US2019/015913.

The transfer vehicles described herein comprise one or more structural lipids. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

The transfer vehicles described herein comprise one or more structural lipids. Incorporation of structural lipids in a transfer vehicle, e.g., a lipid nanoparticle, may help mitigate aggregation of other lipids in the particle. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. Structural lipids can include, but are not limited to, sterols (e.g., phytosterols or zoosterols).

In certain embodiments, the structural lipid is a steroid. For example, sterols can include, but are not limited to, cholesterol, β-sitosterol, fecosterol, ergosterol, sitosterol, campesterol, stigmasterol, brassicasterol, ergosterol, tomatidine, tomatine, ursolic acid, or alpha-tocopherol.

In some embodiments, a transfer vehicle includes an effective amount of an immune cell delivery potentiating lipid, e.g., a cholesterol analog or an amino lipid or combination thereof, that, when present in a transfer vehicle, e.g., an lipid nanoparticle, may function by enhancing cellular association and/or uptake, internalization, intracellular trafficking and/or processing, and/or endosomal escape and/or may enhance recognition by and/or binding to immune cells, relative to a transfer vehicle lacking the immune cell delivery potentiating lipid. Accordingly, while not intending to be bound by any particular mechanism or theory, in one embodiment, a structural lipid or other immune cell delivery potentiating lipid of the disclosure binds to C1q or promotes the binding of a transfer vehicle comprising such lipid to C1q. Thus, for in vitro use of the transfer vehicles of the disclosure for delivery of a nucleic acid molecule to an immune cell, culture conditions that include C1q are used (e.g., use of culture media that includes serum or addition of exogenous C1q to serum-free media). For in vivo use of the transfer vehicles of the disclosure, the requirement for C1q is supplied by endogenous C1q.

In certain embodiments, the structural lipid is cholesterol. In certain embodiments, the structural lipid is an analog of cholesterol. In some embodiments, the structural lipid is a lipid in Table 16:

TABLE 16

| CMPD | |
|---|---|
| No. S- | Structure |
| 1 | 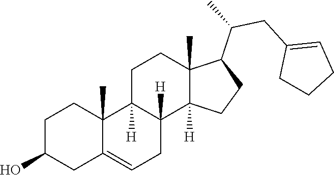 |
| 2 | 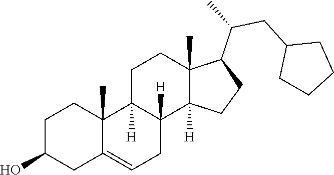 |
| 3 | 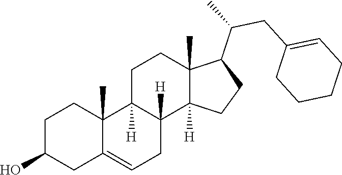 |
| 4 | 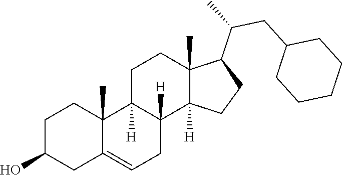 |
| 5 | 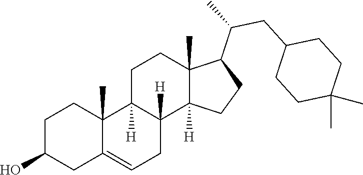 |
| 6 | 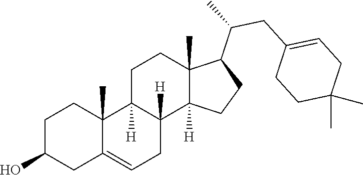 |
| 7 | 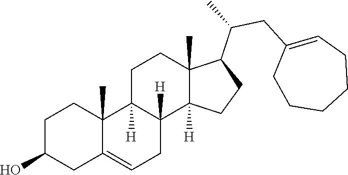 |
| 8 | 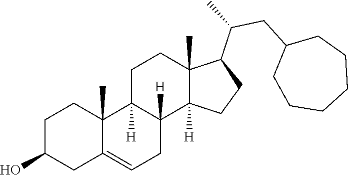 |
| 9 | 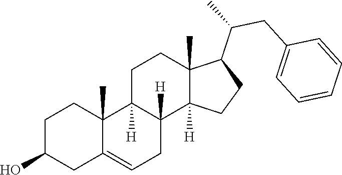 |
| 10 | 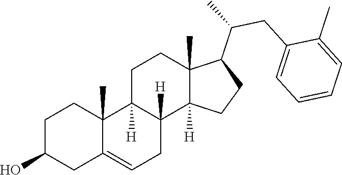 |
| 11 | 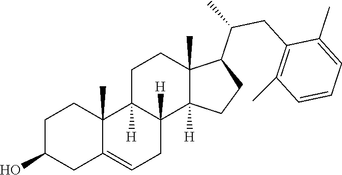 |
| 12 | 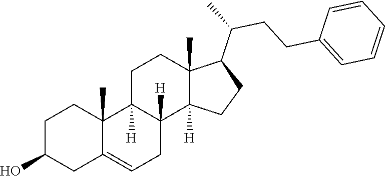 |
| 13 | 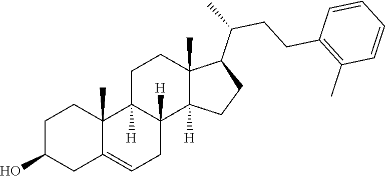 |
| 14 | 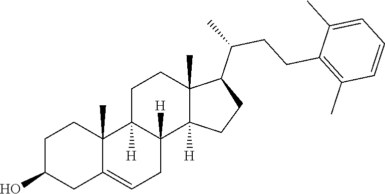 |
| 15 | 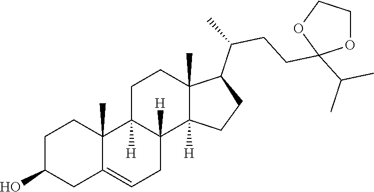 |
| 16 | 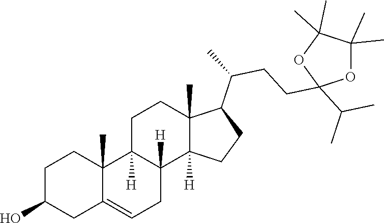 |
| 17 | 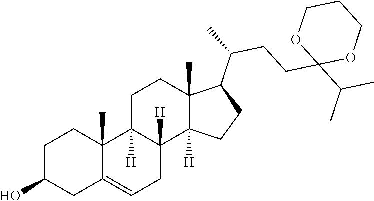 |
| 18 | 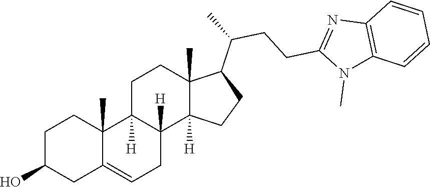 |
| 19 | 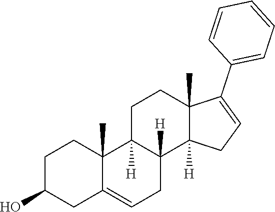 |
| 20 | 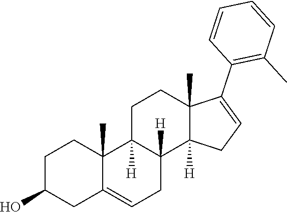 |
| 21 | 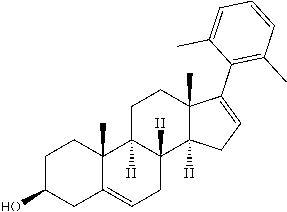 |
| 22 | 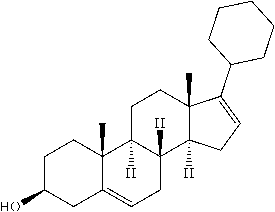 |
| 23 | 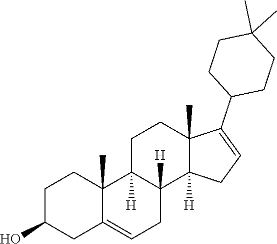 |
| 24 | 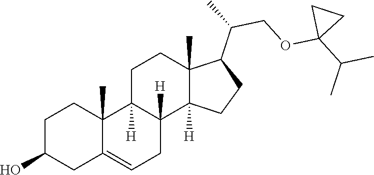 |
| 25 | 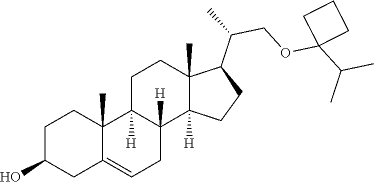 |
| 26 | 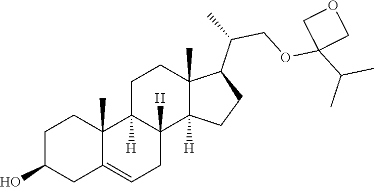 |
| 27 | 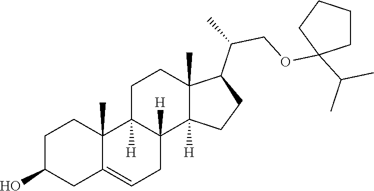 |
| 28 | 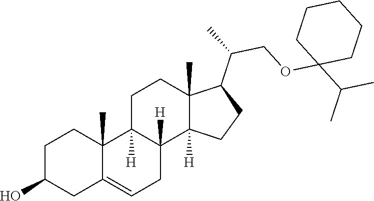 |
| 29 | 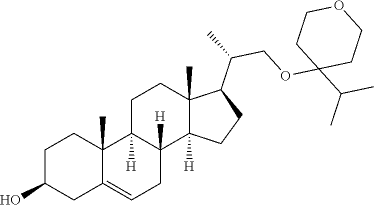 |
| 30 | 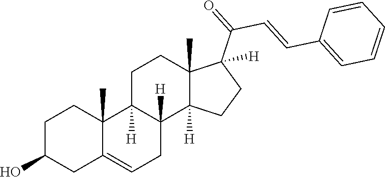 |
| 31 | 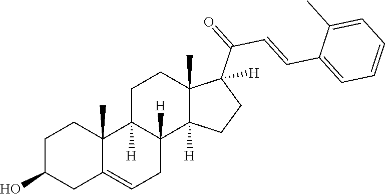 |
| 32 | 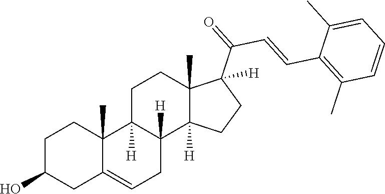 |
| 33 | 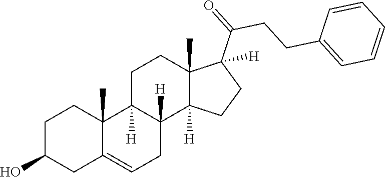 |
| 34 | 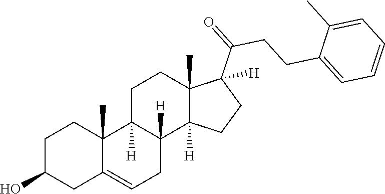 |
| 35 | 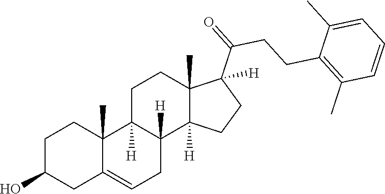 |
| 36 | 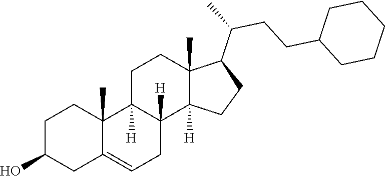 |
| 37 | 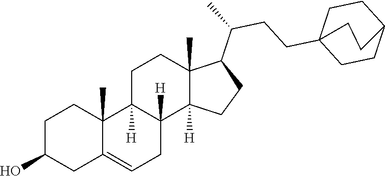 |
| 38 | 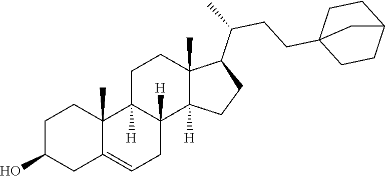 |
| 39 | 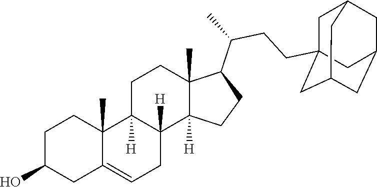 |
| 40 | 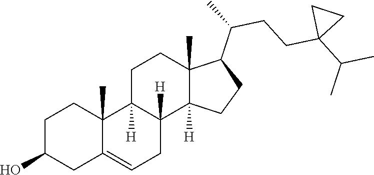 |
| 41 | 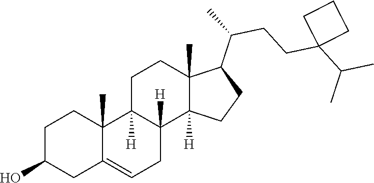 |
| 42 | 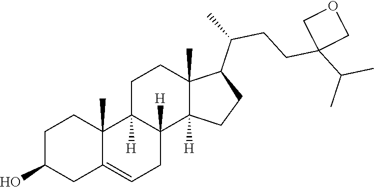 |
| 43 | 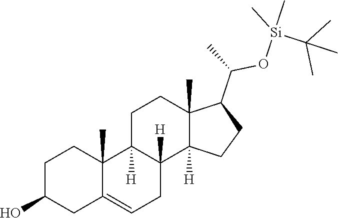 |
| 44 | 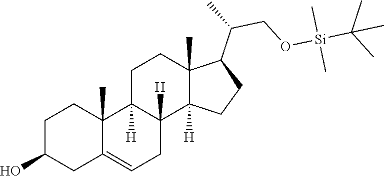 |
| 45 | 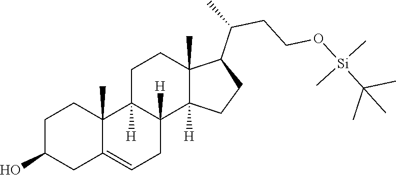 |
| 46 | 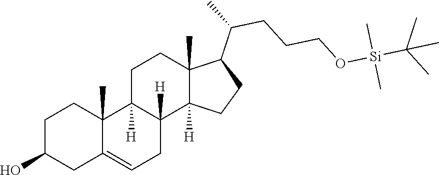 |
| 47 | 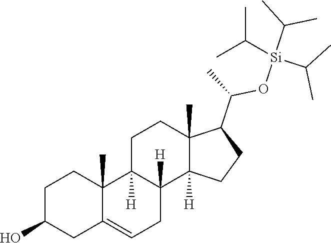 |
| 48 | 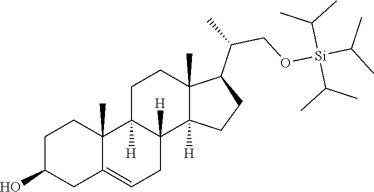 |
| 49 | 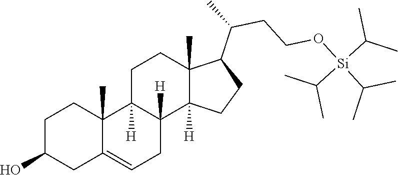 |
| 50 | 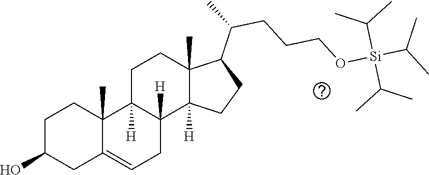 |
| 51 | 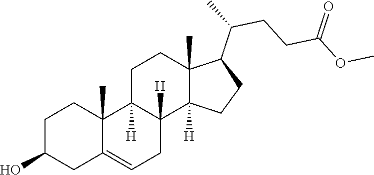 |
| 52 | 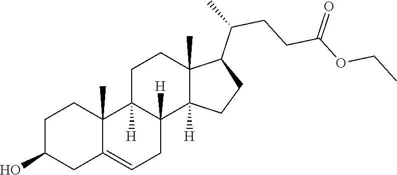 |
| 53 | 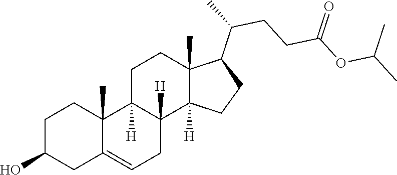 |
| 54 | 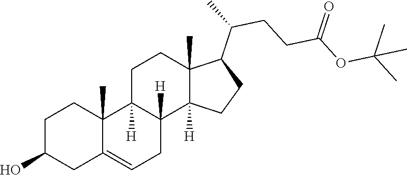 |
| 55 | 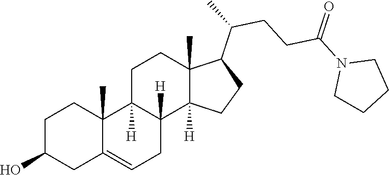 |
| 56 | 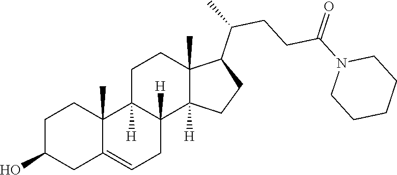 |
| 57 | 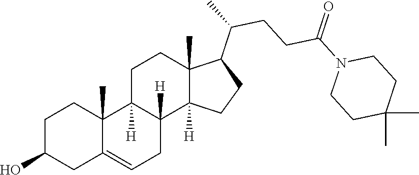 |
| 58 | 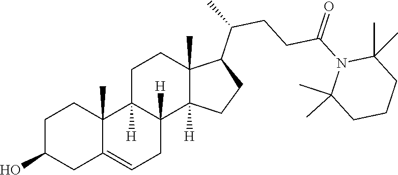 |
| 59 | 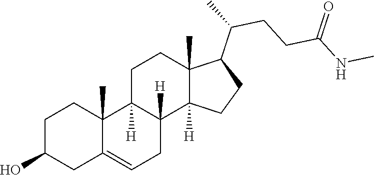 |
| 60 | 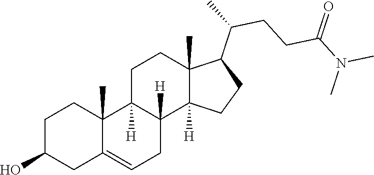 |
| 61 | 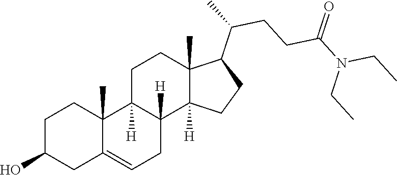 |
| 62 | 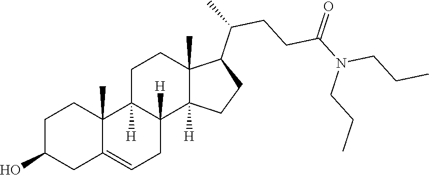 |
| 63 | 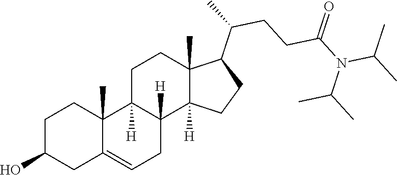 |
| 64 | 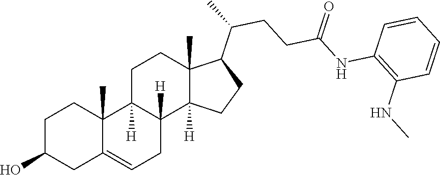 |
| 65 | 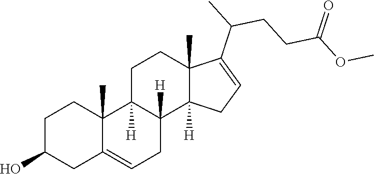 |
| 66 | 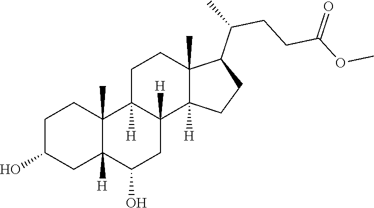 |
| 67 | 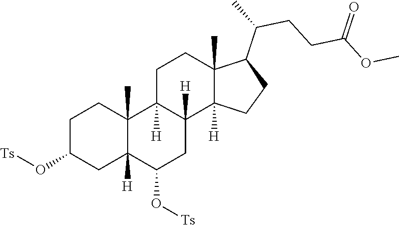 |
| 68 | 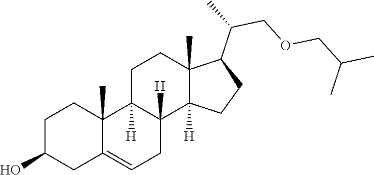 |
| 69 | 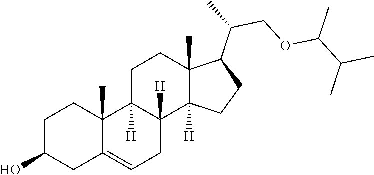 |
| 70 | 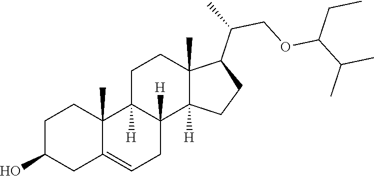 |
| 71 | 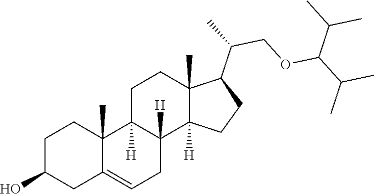 |
| 72 | 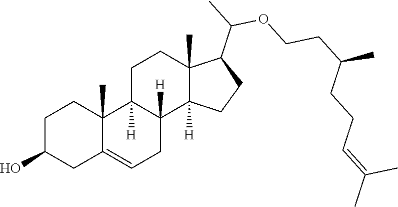 |
| 73 | 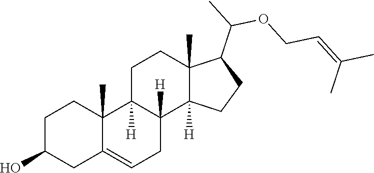 |
| 74 | 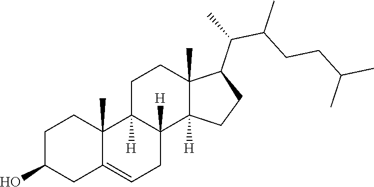 |
| 75 | 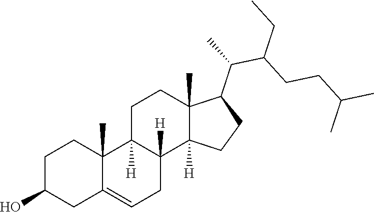 |
| 76 | 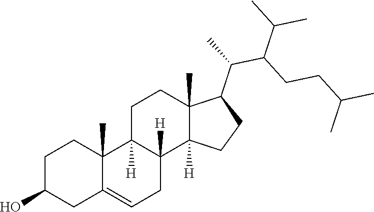 |
| 77 | 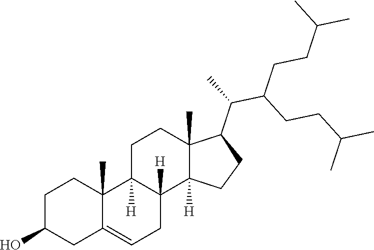 |
| 78 | 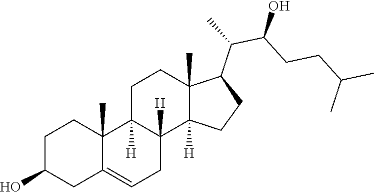 |
| 79 | 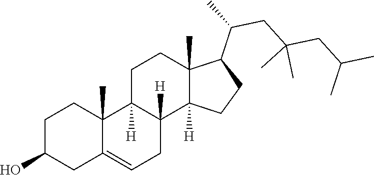 |
| 80 | 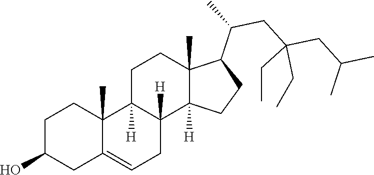 |
| 81 | 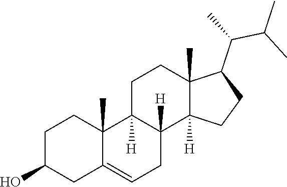 |
| 82 | 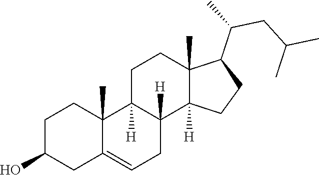 |
| 83 | 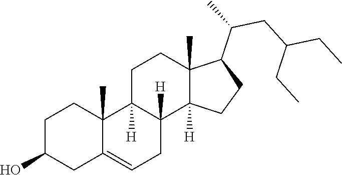 |
| 84 | 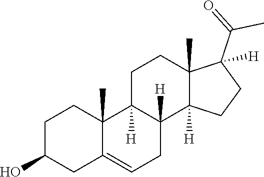 |
| 85 | 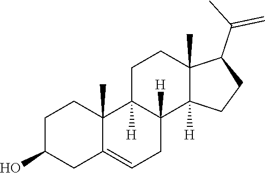 |
| 86 | 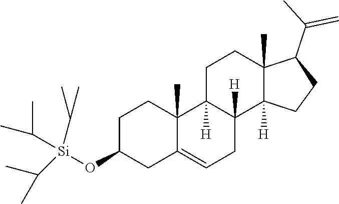 |
| 87 | 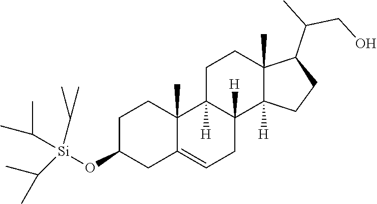 |
| 88 | 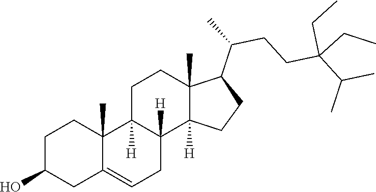 |
| 89 | 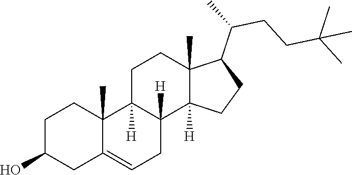 |
| 90 | 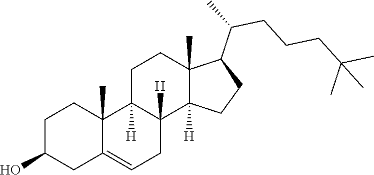 |
| 91 | 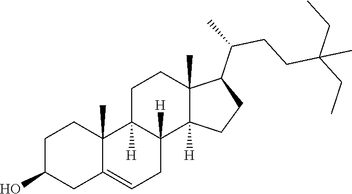 |
| 92 | 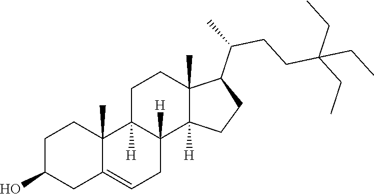 |
| 93 | 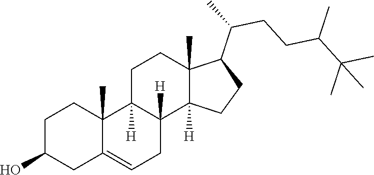 |
| 94 | 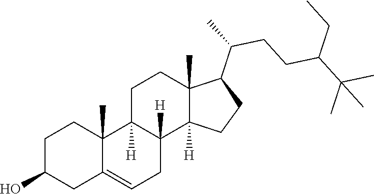 |
| 95 | 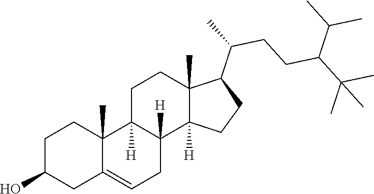 |
| 96 | 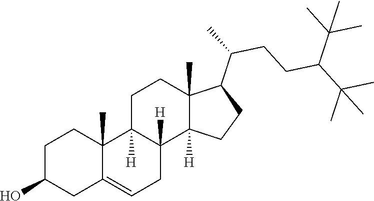 |
| 97 | 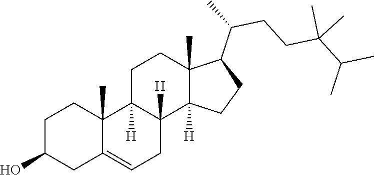 |
| 98 | 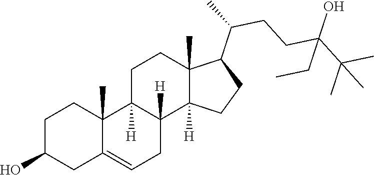 |
| 99 | 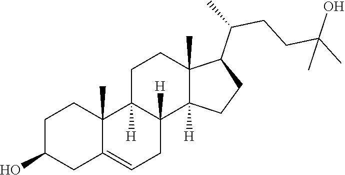 |
| 100 | 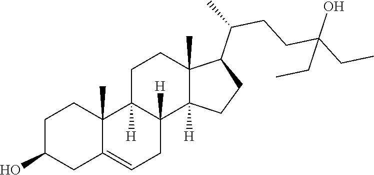 |
| 101 | 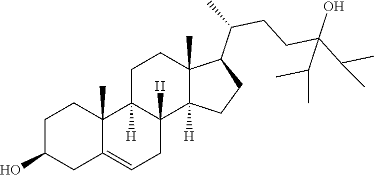 |
| 102 | 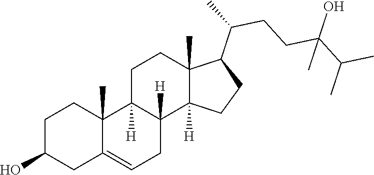 |
| 103 | 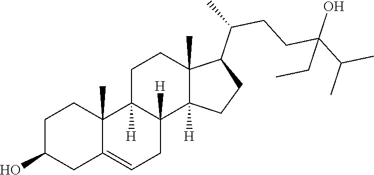 |
| 104 | 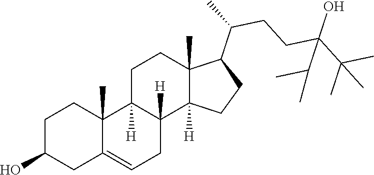 |
| 105 | 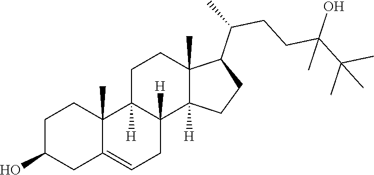 |
| 106 | 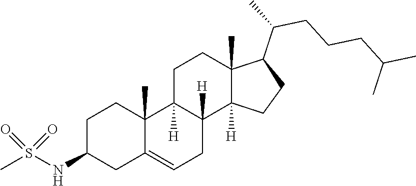 |
| 107 | 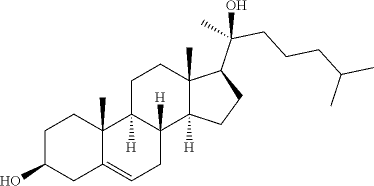 |
| 108 | 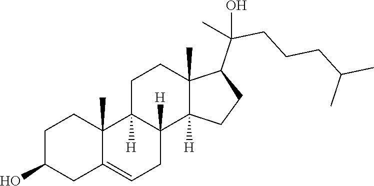 |
| 109 | 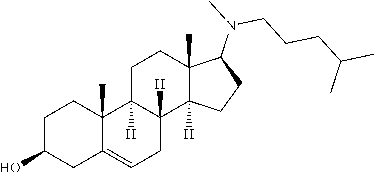 |
| 110 | 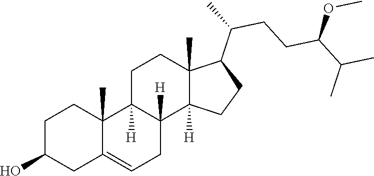 |
| 111 | 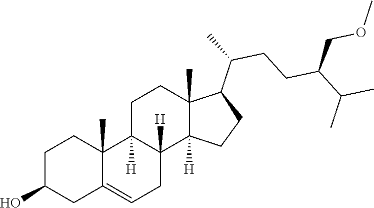 |
| 112 | 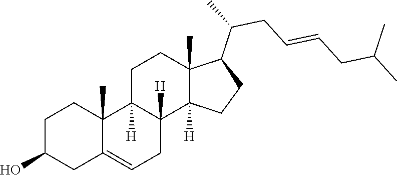 |
| 113 | 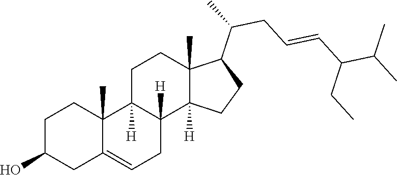 |
| 114 | 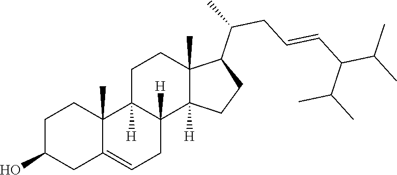 |
| 115 | 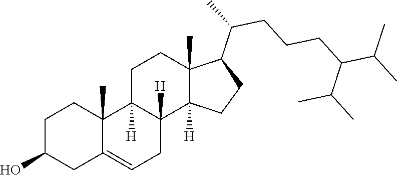 |
| 116 | 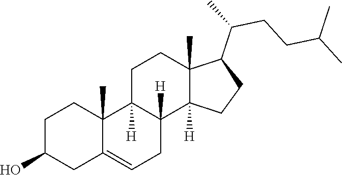 |
| 117 | 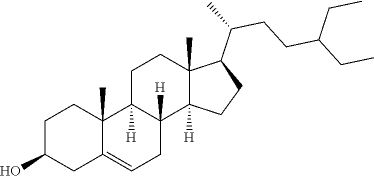 |
| 118 | 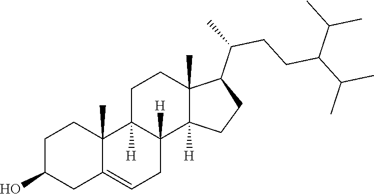 |
| 119 | 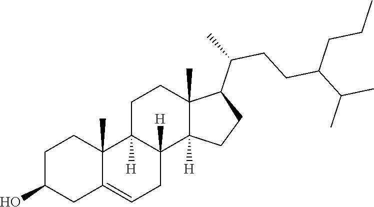 |
| 120 | 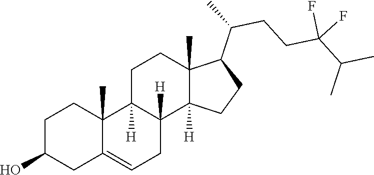 |
| 121 | 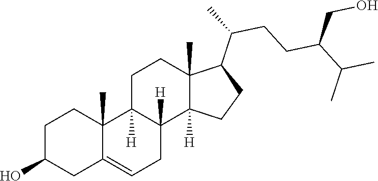 |
| 122 | 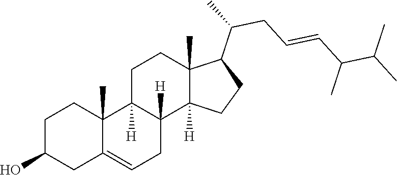 |
| 123 | 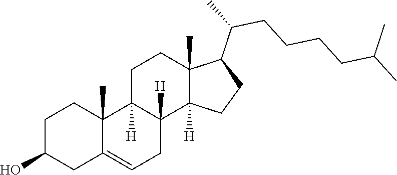 |
| 124 | 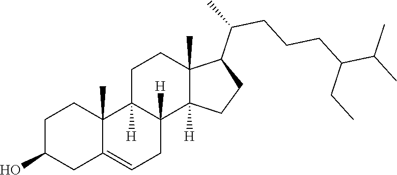 |
| 125 | 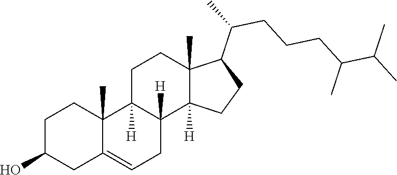 |
| 126 | 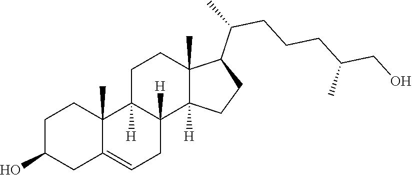 |
| 127 | 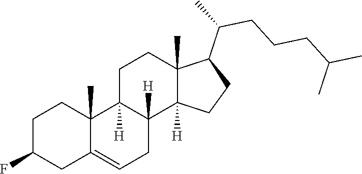 |
| 128 | 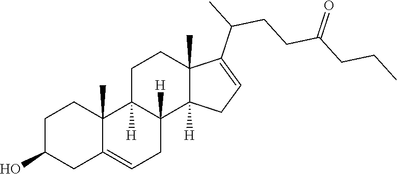 |
| 129 | 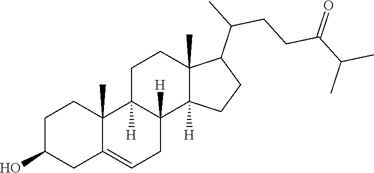 |
| 130 | 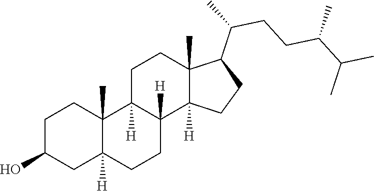 |
| 131 | 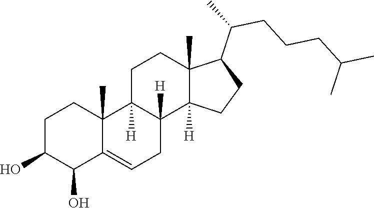 |
| 132 | 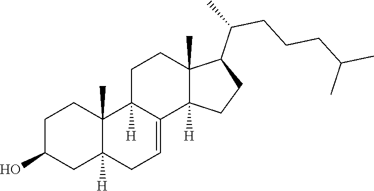 |
| 133 | 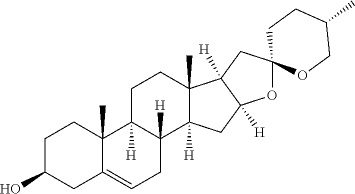 |
| 134 | 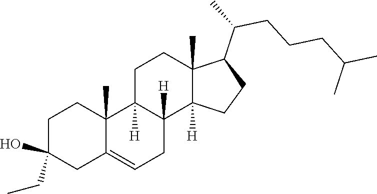 |
| 135 | 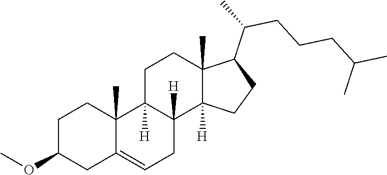 |
| 136 | 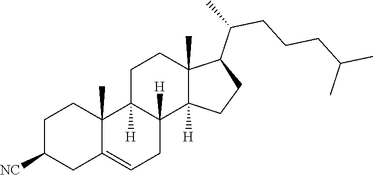 |
| 137 | 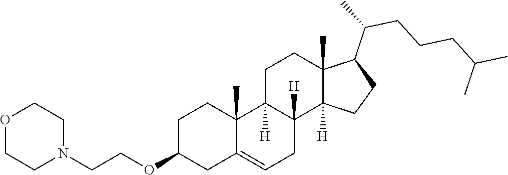 |
| 138 | 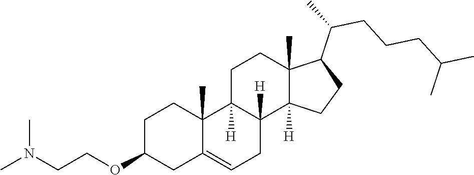 |
| 139 | 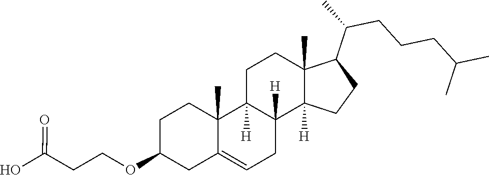 |
| 140 | 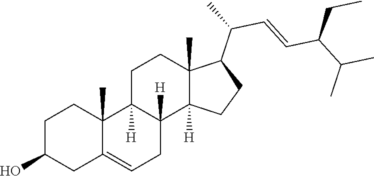 |
| 141 | 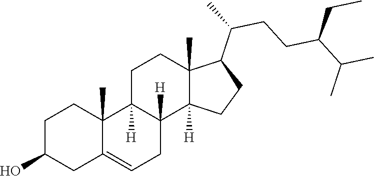 |
| 142 | 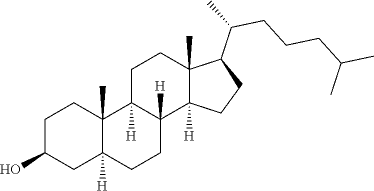 |
| 143 | 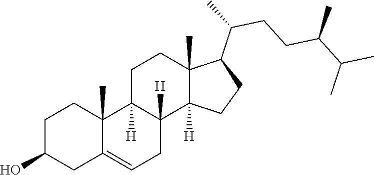 |
| 144 | 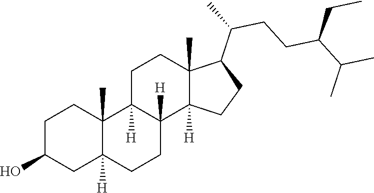 |
| 145 | 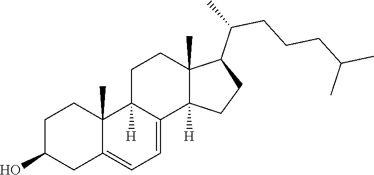 |
| 146 | 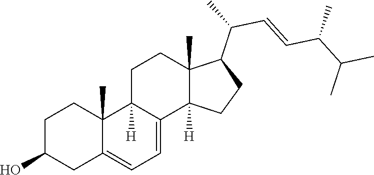 |
| 147 | 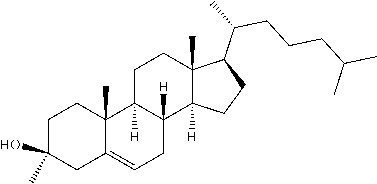 |
| 148 | 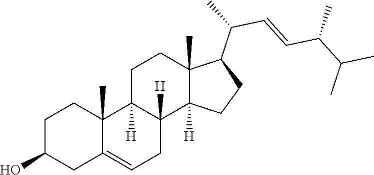 |
| 149 | 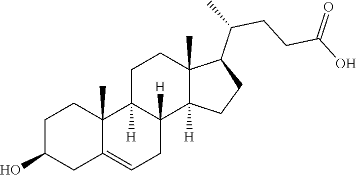 |
| 150 | 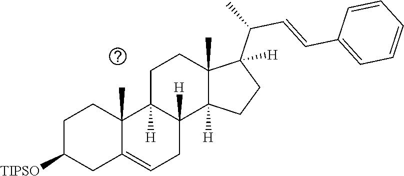 |
| 151 | 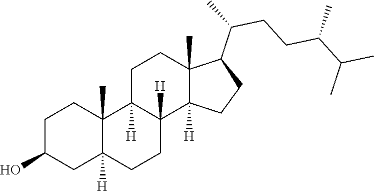 |
| 152 | 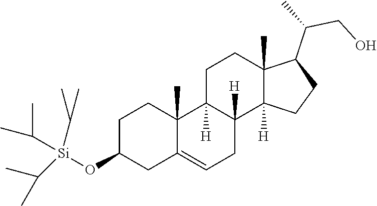 |
| 153 | 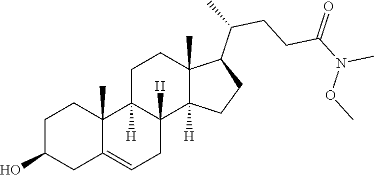 |
| 154 | 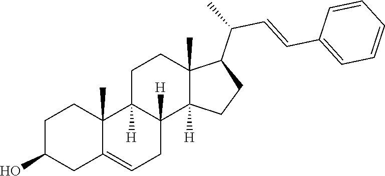 |
| 155 | 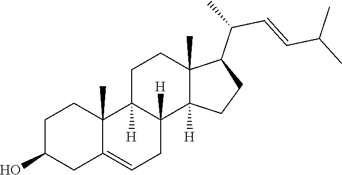 |
| 156 | 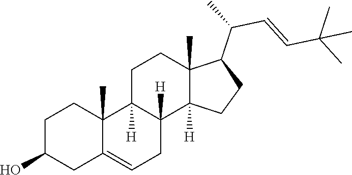 |
| 157 | 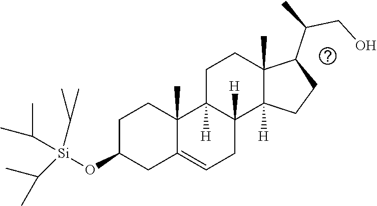 |
| 158 | 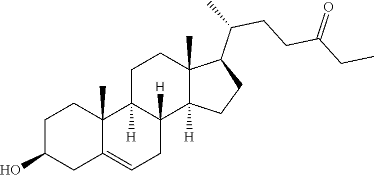 |
| 159 | 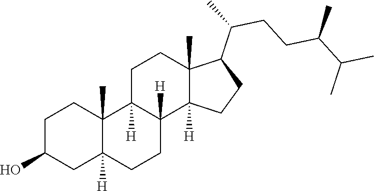 |
| 160 | 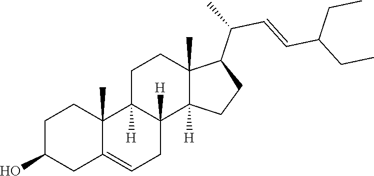 |
| 161 | 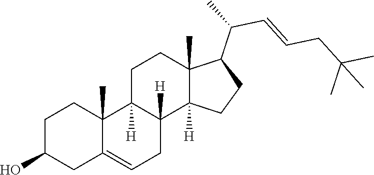 |
| 162 | 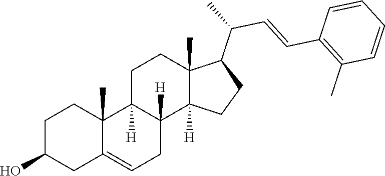 |
| 163 | 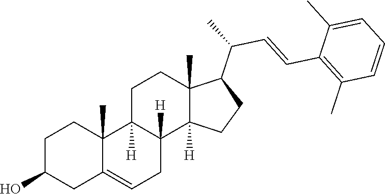 |
| 164 | 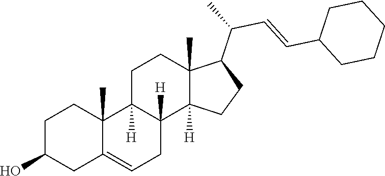 |
| 165 | 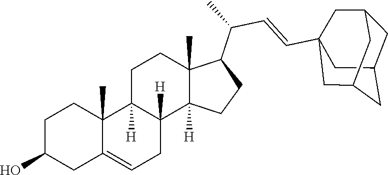 |
| 166 | 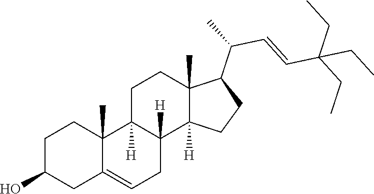 |
| 167 | 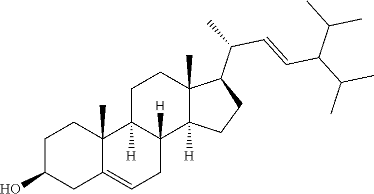 |
| 168 | 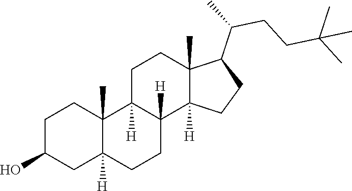 |
| 169 | 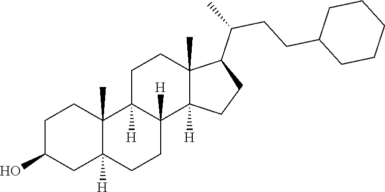 |
| 170 | 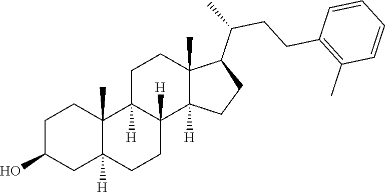 |
| 171 | 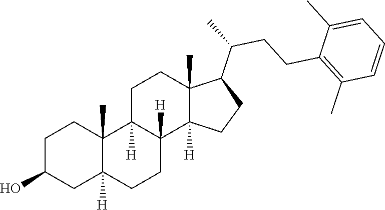 |
| 172 | 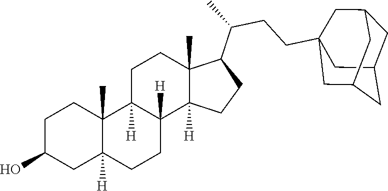 |
| 173 | 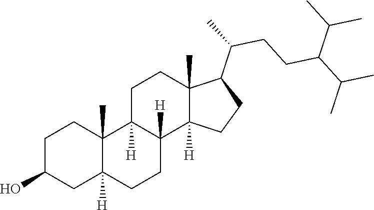 |
| 174 | 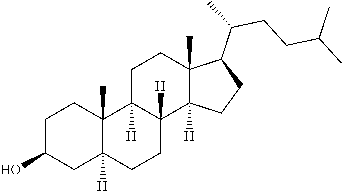 |
| 175 | 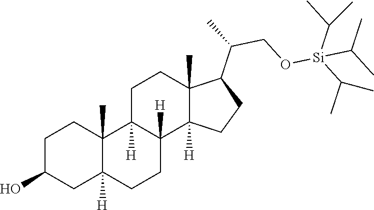 |
| 176 | 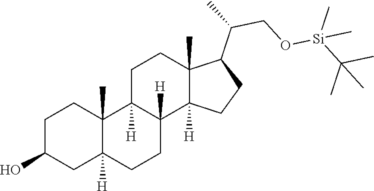 |
| 177 | 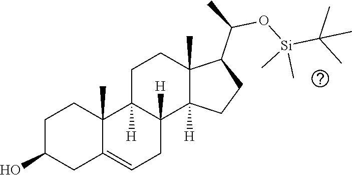 |
| 178 | 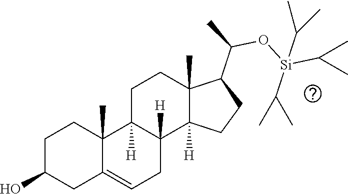 |
| 179 | 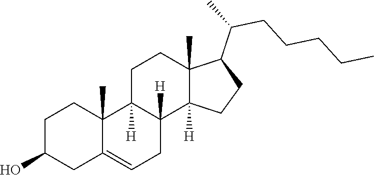 |
| 180 | 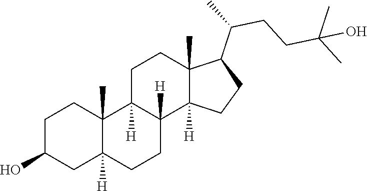 |
| 181 | 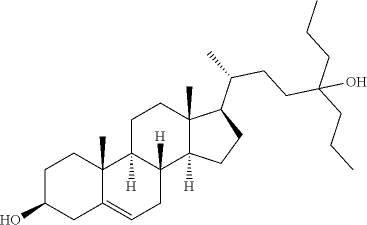 |
| 182 | 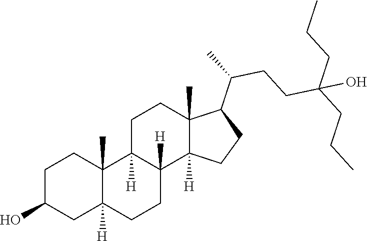 |
| 184 | 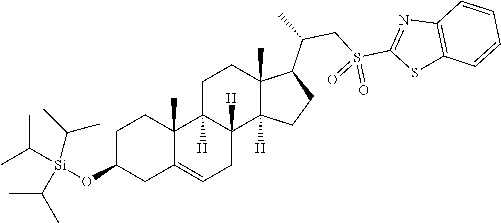 |
| Composition | |
| S- No. | Structure |
| 183 | 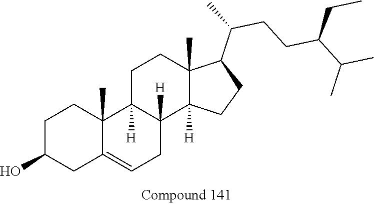 |
| | 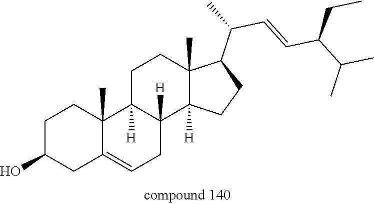 |
| | 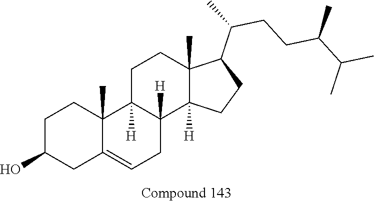 |
| | 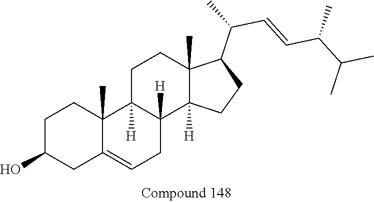 |

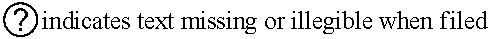

### 4. LNP Formulations

The formation of a lipid nanoparticle (LNP) described herein may be accomplished by any methods known in the art. For example, as described in U.S. Pat. Pub. No. US2012/0178702 A1, which is incorporated herein by reference in its entirety. Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51:8529-8533; and Maier et al. (2013) Molecular Therapy 21, 1570-1578 (the contents of each of which are incorporated herein by reference in their entirety).

In one embodiment, the LNP formulation may be prepared by, e.g., the methods described in International Pat. Pub. No. WO 2011/127255 or WO 2008/103276, the contents of each of which are herein incorporated by reference in their entirety.

In one embodiment, LNP formulations described herein may comprise a polycationic composition. As a non-limiting example, the polycationic composition may be a composition selected from Formulae 1-60 of U.S. Pat. Pub. No. US2005/0222064 A1, the content of which is herein incorporated by reference in its entirety.

In one embodiment, the lipid nanoparticle may be formulated by the methods described in U.S. Pat. Pub. No. US2013/0156845 A1, and International Pat. Pub. No. WO2013/093648 A2 or WO2012/024526 A2, each of which is herein incorporated by reference in its entirety.

In one embodiment, the lipid nanoparticles described herein may be made in a sterile environment by the system and/or methods described in U.S. Pat. Pub. No. US2013/0164400 A1, which is incorporated herein by reference in its entirety.

In one embodiment, the LNP formulation may be formulated in a nanoparticle such as a nucleic acid-lipid particle described in U.S. Pat. No. 8,492,359, which is incorporated herein by reference in its entirety.

A nanoparticle composition may optionally comprise one or more coatings. For example, a nanoparticle composition may be formulated in a capsule, film, or tablet having a coating. A capsule, film, or tablet including a composition described herein may have any useful size, tensile strength, hardness, or density.

In some embodiments, the lipid nanoparticles described herein may be synthesized using methods comprising microfluidic mixers. Exemplary microfluidic mixers may include, but are not limited to, a slit interdigitial micromixer including, but not limited to, those manufactured by Precision Nanosystems (Vancouver, BC, Canada), Microinnova (Allerheiligen bei Wildon, Austria) and/or a staggered herringbone micromixer (SHM) (Zhigaltsev, I. V. et al. (2012) Langmuir. 28:3633-40; Belliveau, N. M. et al. Mol. Ther. Nucleic. Acids. (2012) 1:e37; Chen, D. et al. J. Am. Chem. Soc. (2012) 134(16):6948-51; each of which is herein incorporated by reference in its entirety).

In some embodiments, methods of LNP generation comprising SHM, further comprise the mixing of at least two input streams wherein mixing occurs by microstructure-induced chaotic advection (MICA). According to this method, fluid streams flow through channels present in a herringbone pattern causing rotational flow and folding the fluids around each other. This method may also comprise a surface for fluid mixing wherein the surface changes orientations during fluid cycling. Methods of generating LNPs using SHM include those disclosed in U.S. Pat. Pub. Nos. US2004/0262223 A1 and US2012/0276209 A1, each of which is incorporated herein by reference in their entirety.

In one embodiment, the lipid nanoparticles may be formulated using a micromixer such as, but not limited to, a Slit Interdigital Microstructured Mixer (SIMM-V2) or a Standard Slit Interdigital Micro Mixer (SSIMM) or Caterpillar (CPMM) or Impinging jet (IJMM) from the Institut fur Mikrotechnik Mainz GmbH, Mainz Germany). In one embodiment, the lipid nanoparticles are created using microfluidic technology (see, Whitesides (2006) Nature. 442: 368-373; and Abraham et al. (2002) Science. 295: 647-651; each of which is herein incorporated by reference in its entirety). As a non-limiting example, controlled microfluidic formulation includes a passive method for mixing streams of steady pressure-driven flows in micro channels at a low Reynolds number (see, e.g., Abraham et al. (2002) Science. 295: 647651; which is herein incorporated by reference in its entirety).

In one embodiment, the circRNA of the present invention may be formulated in lipid nanoparticles created using a micromixer chip such as, but not limited to, those from Harvard Apparatus (Holliston, Mass.), Dolomite Microfluidics (Royston, UK), or Precision Nanosystems (Van Couver, BC, Canada). A micromixer chip can be used for rapid mixing of two or more fluid streams with a split and recombine mechanism.

In one embodiment, the lipid nanoparticles may have a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm. In one embodiment, the lipid nanoparticles may have a diameter from about 10 to 500 nm. In one embodiment, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm. Each possibility represents a separate embodiment of the present invention.

In some embodiments, a nanoparticle (e.g., a lipid nanoparticle) has a mean diameter of 10-500 nm, 20-400 nm, 30-300 nm, or 40-200 nm. In some embodiments, a nanoparticle (e.g., a lipid nanoparticle) has a mean diameter of 50-150 nm, 50-200 nm, 80-100 nm, or 80-200 nm.

In some embodiments, the lipid nanoparticles described herein can have a diameter from below 0.1 μm to up to 1 mm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, less than 975 μm.

In another embodiment, LNPs may have a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm. Each possibility represents a separate embodiment of the present invention.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition may be from about 0.10 to about 0.20. Each possibility represents a separate embodiment of the present invention.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about −20 mV to about +20 mV, from about −20 mV to about +15 mV, from about −20 mV to about +10 mV, from about −20 mV to about +5 mV, from about −20 mV to about 0 mV, from about −20 mV to about −5 mV, from about −20 mV to about −10 mV, from about −20 mV to about −15 mV from about −20 mV to about +20 mV, from about −20 mV to about +15 mV, from about −20 mV to about +10 mV, from about −20 mV to about +5 mV, from about −20 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV. Each possibility represents a separate embodiment of the present invention.

The efficiency of encapsulation of a therapeutic agent describes the amount of therapeutic agent that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of therapeutic agent in a solution containing the nanoparticle composition before and after breaking up the nanoparticle composition with one or more organic solvents or detergents. Fluorescence may be used to measure the amount of free therapeutic agent (e.g., nucleic acids) in a solution. For the nanoparticle compositions described herein, the encapsulation efficiency of a therapeutic agent may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%. Each possibility represents a separate embodiment of the present invention. In some embodiments, the lipid nanoparticle has a polydiversity value of less than 0.4. In some embodiments, the lipid nanoparticle has a net neutral charge at a neutral pH. In some embodiments, the lipid nanoparticle has a mean diameter of 50-200 nm.

The properties of a lipid nanoparticle formulation may be influenced by factors including, but not limited to, the selection of the cationic lipid component, the degree of cationic lipid saturation, the selection of the non-cationic lipid component, the degree of noncationic lipid saturation, the selection of the structural lipid component, the nature of the PEGylation, ratio of all components and biophysical parameters such as size. As described herein, the purity of a PEG lipid component is also important to an LNP's properties and performance.

### 5. Methods

In one embodiment, a lipid nanoparticle formulation may be prepared by the methods described in International Publication Nos. WO2011127255 or WO2008103276, each of which is herein incorporated by reference in their entirety. In some embodiments, lipid nanoparticle formulations may be as described in International Publication No. WO2019131770, which is herein incorporated by reference in its entirety.

In some embodiments, circular RNA is formulated according to a process described in U.S. patent application Ser. No. 15/809,680. In some embodiments, the present invention provides a process of encapsulating circular RNA in transfer vehicles comprising the steps of forming lipids into pre-formed transfer vehicles (i.e. formed in the absence of RNA) and then combining the pre-formed transfer vehicles with RNA. In some embodiments, the novel formulation process results in an RNA formulation with higher potency (peptide or protein expression) and higher efficacy (improvement of a biologically relevant endpoint) both in vitro and in vivo with potentially better tolerability as compared to the same RNA formulation prepared without the step of preforming the lipid nanoparticles (e.g., combining the lipids directly with the RNA).

For certain cationic lipid nanoparticle formulations of RNA, in order to achieve high encapsulation of RNA, the RNA in buffer (e.g., citrate buffer) has to be heated. In those processes or methods, the heating is required to occur before the formulation process (i.e. heating the separate components) as heating post-formulation (post-formation of nanoparticles) does not increase the encapsulation efficiency of the RNA in the lipid nanoparticles. In contrast, in some embodiments of the novel processes of the present invention, the order of heating of RNA does not appear to affect the RNA encapsulation percentage. In some embodiments, no heating (i.e. maintaining at ambient temperature) of one or more of the solutions comprising the pre-formed lipid nanoparticles, the solution comprising the RNA and the mixed solution comprising the lipid nanoparticle encapsulated RNA is required to occur before or after the formulation process.

RNA may be provided in a solution to be mixed with a lipid solution such that the RNA may be encapsulated in lipid nanoparticles. A suitable RNA solution may be any aqueous solution containing RNA to be encapsulated at various concentrations. For example, a suitable RNA solution may contain an RNA at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, or 1.0 mg/ml. In some embodiments, a suitable RNA solution may contain an RNA at a concentration in a range from about 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml.

Typically, a suitable RNA solution may also contain a buffering agent and/or salt. Generally, buffering agents can include HEPES, Tris, ammonium sulfate, sodium bicarbonate, sodium citrate, sodium acetate, potassium phosphate or sodium phosphate. In some embodiments, suitable concentration of the buffering agent may be in a range from about 0.1 mM to 100 mM, 0.5 mM to 90 mM, 1.0 mM to 80 mM, 2 mM to 70 mM, 3 mM to 60 mM, 4 mM to 50 mM, 5 mM to 40 mM, 6 mM to 30 mM, 7 mM to 20 mM, 8 mM to 15 mM, or 9 to 12 mM.

Exemplary salts can include sodium chloride, magnesium chloride, and potassium chloride. In some embodiments, suitable concentration of salts in an RNA solution may be in a range from about 1 mM to 500 mM, 5 mM to 400 mM, 10 mM to 350 mM, 15 mM to 300 mM, 20 mM to 250 mM, 30 mM to 200 mM, 40 mM to 190 mM, 50 mM to 180 mM, 50 mM to 170 mM, 50 mM to 160 mM, 50 mM to 150 mM, or 50 mM to 100 mM.

In some embodiments, a suitable RNA solution may have a pH in a range from about 3.5-6.5, 3.5-6.0, 3.5-5.5, 3.5-5.0, 3.5-4.5, 4.0-5.5, 4.0-5.0, 4.0-4.9, 4.0-4.8, 4.0-4.7, 4.0-4.6, or 4.0-4.5.

Various methods may be used to prepare an RNA solution suitable for the present invention. In some embodiments, RNA may be directly dissolved in a buffer solution described herein. In some embodiments, an RNA solution may be generated by mixing an RNA stock solution with a buffer solution prior to mixing with a lipid solution for encapsulation. In some embodiments, an RNA solution may be generated by mixing an RNA stock solution with a buffer solution immediately before mixing with a lipid solution for encapsulation.

According to the present invention, a lipid solution contains a mixture of lipids suitable to form transfer vehicles for encapsulation of RNA. In some embodiments, a suitable lipid solution is ethanol based. For example, a suitable lipid solution may contain a mixture of desired lipids dissolved in pure ethanol (i.e. 100% ethanol). In another embodiment, a suitable lipid solution is isopropyl alcohol based. In another embodiment, a suitable lipid solution is dimethylsulfoxide-based. In another embodiment, a suitable lipid solution is a mixture of suitable solvents including, but not limited to, ethanol, isopropyl alcohol and dimethylsulfoxide.

A suitable lipid solution may contain a mixture of desired lipids at various concentrations. In some embodiments, a suitable lipid solution may contain a mixture of desired lipids at a total concentration in a range from about 0.1-100 mg/ml, 0.5-90 mg/ml, 1.0-80 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml.

### 6. Targeting

The present invention also contemplates the discriminatory targeting of target cells and tissues by both passive and active targeting means. The phenomenon of passive targeting exploits the natural distributions patterns of a transfer vehicle in vivo without relying upon the use of additional excipients or means to enhance recognition of the transfer vehicle by target cells. For example, transfer vehicles which are subject to phagocytosis by the cells of the reticulo-endothelial system are likely to accumulate in the liver or spleen, and accordingly may provide a means to passively direct the delivery of the compositions to such target cells.

Alternatively, the present invention contemplates active targeting, which involves the use of targeting moieties that may be bound (either covalently or non-covalently) to the transfer vehicle to encourage localization of such transfer vehicle at certain target cells or target tissues. For example, targeting may be mediated by the inclusion of one or more endogenous targeting moieties in or on the transfer vehicle to encourage distribution to the target cells or tissues. Recognition of the targeting moiety by the target tissues actively facilitates tissue distribution and cellular uptake of the transfer vehicle and/or its contents in the target cells and tissues (e.g., the inclusion of an apolipoprotein-E targeting ligand in or on the transfer vehicle encourages recognition and binding of the transfer vehicle to endogenous low density lipoprotein receptors expressed by hepatocytes). As provided herein, the composition can comprise a moiety capable of enhancing affinity of the composition to the target cell. Targeting moieties may be linked to the outer bilayer of the lipid particle during formulation or post-formulation. These methods are well known in the art. In addition, some lipid particle formulations may employ fusogenic polymers such as PEAA, hemaglutinin, other lipopeptides (see U.S. patent application Ser. No. 08/835,281, and 60/083,294, which are incorporated herein by reference) and other features useful for in vivo and/or intracellular delivery. In other some embodiments, the compositions of the present invention demonstrate improved transfection efficacies, and/or demonstrate enhanced selectivity towards target cells or tissues of interest. Contemplated therefore are compositions which comprise one or more moieties (e.g., peptides, aptamers, oligonucleotides, a vitamin or other molecules) that are capable of enhancing the affinity of the compositions and their nucleic acid contents for the target cells or tissues. Suitable moieties may optionally be bound or linked to the surface of the transfer vehicle. In some embodiments, the targeting moiety may span the surface of a transfer vehicle or be encapsulated within the transfer vehicle. Suitable moieties and are selected based upon their physical, chemical or biological properties (e.g., selective affinity and/or recognition of target cell surface markers or features). Cell-specific target sites and their corresponding targeting ligand can vary widely. Suitable targeting moieties are selected such that the unique characteristics of a target cell are exploited, thus allowing the composition to discriminate between target and non-target cells. For example, compositions of the invention may include surface markers (e.g., apolipoprotein-B or apolipoprotein-E) that selectively enhance recognition of, or affinity to hepatocytes (e.g., by receptor-mediated recognition of and binding to such surface markers). As an example, the use of galactose as a targeting moiety would be expected to direct the compositions of the present invention to parenchymal hepatocytes, or alternatively the use of mannose containing sugar residues as a targeting ligand would be expected to direct the compositions of the present invention to liver endothelial cells (e.g., mannose containing sugar residues that may bind preferentially to the asialoglycoprotein receptor present in hepatocytes). (See Hillery A M, et al. “Drug Delivery and Targeting: For Pharmacists and Pharmaceutical Scientists” (2002) Taylor & Francis, Inc.) The presentation of such targeting moieties that have been conjugated to moieties present in the transfer vehicle (e.g., a lipid nanoparticle) therefore facilitate recognition and uptake of the compositions of the present invention in target cells and tissues. Examples of suitable targeting moieties include one or more peptides, proteins, aptamers, vitamins and oligonucleotides.

In particular embodiments, a transfer vehicle comprises a targeting moiety. In some embodiments, the targeting moiety mediates receptor-mediated endocytosis selectively into a specific population of cells. In some embodiments, the targeting moiety is capable of binding to a T cell antigen. In some embodiments, the targeting moiety is capable of binding to a NK, NKT, or macrophage antigen. In some embodiments, the targeting moiety is capable of binding to a protein selected from the group CD3, CD4, CD8, PD-1, 4-1BB, and CD2. In some embodiments, the targeting moiety is an single chain Fv (scFv) fragment, nanobody, peptide, peptide-based macrocycle, minibody, heavy chain variable region, light chain variable region or fragment thereof. In some embodiments, the targeting moiety is selected from T-cell receptor motif antibodies, T-cell a chain antibodies, T-cell β chain antibodies, T-cell y chain antibodies, T-cell δ chain antibodies, CCR7 antibodies, CD3 antibodies, CD4 antibodies, CD5 antibodies, CD7 antibodies, CD8 antibodies, CD11b antibodies, CD11c antibodies, CD16 antibodies, CD19 antibodies, CD20 antibodies, CD21 antibodies, CD22 antibodies, CD25 antibodies, CD28 antibodies, CD34 antibodies, CD35 antibodies, CD40 antibodies, CD45RA antibodies, CD45RO antibodies, CD52 antibodies, CD56 antibodies, CD62L antibodies, CD68 antibodies, CD80 antibodies, CD95 antibodies, CD117 antibodies, CD127 antibodies, CD133 antibodies, CD137 (4-1BB) antibodies, CD163 antibodies, F4/80 antibodies, IL-4Ra antibodies, Sca-1 antibodies, CTLA-4 antibodies, GITR antibodies GARP antibodies, LAP antibodies, granzyme B antibodies, LFA-1 antibodies, transferrin receptor antibodies, and fragments thereof. In some embodiments, the targeting moiety is a small molecule binder of an ectoenzyme on lymphocytes. Small molecule binders of ectoenzymes include A2A inhibitors CD73 inhibitors, CD39 or adesines receptors A2aR and A2bR. Potential small molecules include AB928.

In some embodiments, transfer vehicles are formulated and/or targeted as described in Shobaki N, Sato Y, Harashima H. Mixing lipids to manipulate the ionization status of lipid nanoparticles for specific tissue targeting. Int J Nanomedicine. 2018; 13:8395-8410. Published 2018 Dec. 10. In some embodiments, a transfer vehicle is made up of 3 lipid types. In some embodiments, a transfer vehicle is made up of 4 lipid types. In some embodiments, a transfer vehicle is made up of 5 lipid types. In some embodiments, a transfer vehicle is made up of 6 lipid types.

### 7. Target Cells

Where it is desired to deliver a nucleic acid to an immune cell, the immune cell represents the target cell. In some embodiments, the compositions of the invention transfect the target cells on a discriminatory basis (i.e., do not transfect non-target cells). The compositions of the invention may also be prepared to preferentially target a variety of target cells, which include, but are not limited to, T cells, B cells, macrophages, and dendritic cells.

In some embodiments, the target cells are deficient in a protein or enzyme of interest. For example, where it is desired to deliver a nucleic acid to a hepatocyte, the hepatocyte represents the target cell. In some embodiments, the compositions of the invention transfect the target cells on a discriminatory basis (i.e., do not transfect non-target cells). The compositions of the invention may also be prepared to preferentially target a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

The compositions of the invention may be prepared to preferentially distribute to target cells such as in the heart, lungs, kidneys, liver, and spleen. In some embodiments, the compositions of the invention distribute into the cells of the liver or spleen to facilitate the delivery and the subsequent expression of the circRNA comprised therein by the cells of the liver (e.g., hepatocytes) or the cells of spleen (e.g., immune cells). The targeted cells may function as a biological “reservoir” or “depot” capable of producing, and systemically excreting a functional protein or enzyme. Accordingly, in one embodiment of the invention the transfer vehicle may target hepatocytes or immune cells and/or preferentially distribute to the cells of the liver or spleen upon delivery. In an embodiment, following transfection of the target hepatocytes or immune cells, the circRNA loaded in the vehicle are translated and a functional protein product is produced, excreted and systemically distributed. In other embodiments, cells other than hepatocytes (e.g., lung, spleen, heart, ocular, or cells of the central nervous system) can serve as a depot location for protein production.

In one embodiment, the compositions of the invention facilitate a subject's endogenous production of one or more functional proteins and/or enzymes. In an embodiment of the present invention, the transfer vehicles comprise circRNA which encode a deficient protein or enzyme. Upon distribution of such compositions to the target tissues and the subsequent transfection of such target cells, the exogenous circRNA loaded into the transfer vehicle (e.g., a lipid nanoparticle) may be translated in vivo to produce a functional protein or enzyme encoded by the exogenously administered circRNA (e.g., a protein or enzyme in which the subject is deficient). Accordingly, the compositions of the present invention exploit a subject's ability to translate exogenously- or recombinantly-prepared circRNA to produce an endogenously-translated protein or enzyme, and thereby produce (and where applicable excrete) a functional protein or enzyme. The expressed or translated proteins or enzymes may also be characterized by the in vivo inclusion of native post-translational modifications which may often be absent in recombinantly-prepared proteins or enzymes, thereby further reducing the immunogenicity of the translated protein or enzyme.

The administration of circRNA encoding a deficient protein or enzyme avoids the need to deliver the nucleic acids to specific organelles within a target cell. Rather, upon transfection of a target cell and delivery of the nucleic acids to the cytoplasm of the target cell, the circRNA contents of a transfer vehicle may be translated and a functional protein or enzyme expressed.

In some embodiments, a circular RNA comprises one or more miRNA binding sites. In some embodiments, a circular RNA comprises one or more miRNA binding sites recognized by miRNA present in one or more non-target cells or non-target cell types (e.g., Kupffer cells or hepatic cells) and not present in one or more target cells or target cell types (e.g., hepatocytes or T cells). In some embodiments, a circular RNA comprises one or more miRNA binding sites recognized by miRNA present in an increased concentration in one or more non-target cells or non-target cell types (e.g., Kupffer cells or hepatic cells) compared to one or more target cells or target cell types (e.g., hepatocytes or T cells). miRNAs are thought to function by pairing with complementary sequences within RNA molecules, resulting in gene silencing.

### 8. Pharmaceutical Compositions

In certain embodiments, provided herein are compositions (e.g., pharmaceutical compositions) comprising a therapeutic agent provided herein. In some embodiments, the therapeutic agent is a circular RNA polynucleotide provided herein. In some embodiments the therapeutic agent is a vector provided herein. In some embodiments, the therapeutic agent is a cell comprising a circular RNA or vector provided herein (e.g., a human cell, such as a human T cell). In certain embodiments, the composition further comprises a pharmaceutically acceptable carrier. In some embodiments, the compositions provided herein comprise a therapeutic agent provided herein in combination with other pharmaceutically active agents or drugs, such as anti-inflammatory drugs or antibodies capable of targeting B cell antigens, e.g., anti-CD20 antibodies, e.g., rituximab.

With respect to pharmaceutical compositions, the pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the therapeutic agent(s) and one which has no detrimental side effects or toxicity under the conditions of use.

The choice of carrier will be determined in part by the particular therapeutic agent, as well as by the particular method used to administer the therapeutic agent. Accordingly, there are a variety of suitable formulations of the pharmaceutical compositions provided herein.

In certain embodiments, the pharmaceutical composition comprises a preservative. In certain embodiments, suitable preservatives may include, for example, methylparaben, propylparaben, sodium benzoate, and benzalkonium chloride. Optionally, a mixture of two or more preservatives may be used. The preservative or mixtures thereof are typically present in an amount of about 0.0001% to about 2% by weight of the total composition.

In some embodiments, the pharmaceutical composition comprises a buffering agent. In some embodiments, suitable buffering agents may include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition.

In some embodiments, the concentration of therapeutic agent in the pharmaceutical composition can vary, e.g., less than about 1%, or at least about 1%, 2%, 3%, 4%, 5%, 6%, 7%, 8%, 9% 10%, 15%, 20%, 25%, 30%, 35%, 40%, 45%, or about 50% or more by weight, and can be selected primarily by fluid volumes, and viscosities, in accordance with the particular mode of administration selected.

The following formulations for oral, aerosol, parenteral (e.g., subcutaneous, intravenous, intraarterial, intramuscular, intradermal, intraperitoneal, and intrathecal), and topical administration are merely exemplary and are in no way limiting. More than one route can be used to administer the therapeutic agents provided herein, and in certain instances, a particular route can provide a more immediate and more effective response than another route.

Formulations suitable for oral administration can comprise or consist of (a) liquid solutions, such as an effective amount of the therapeutic agent dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol and the polyethylene alcohols, either with or without the addition of a pharmaceutically acceptable surfactant. Capsule forms can be of the ordinary hard or soft shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the therapeutic agent with a flavorant, usually sucrose, acacia or tragacanth. Pastilles can comprise the therapeutic agent with an inert base, such as gelatin and glycerin, or sucrose and acacia, emulsions, gels, and the like containing, in addition to, such excipients as are known in the art.

Formulations suitable for parenteral administration include aqueous and nonaqueous isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In some embodiments, the therapeutic agents provided herein can be administered in a physiologically acceptable diluent in a pharmaceutical carrier, such as a sterile liquid or mixture of liquids, including water, saline, aqueous dextrose and related sugar solutions, an alcohol such as ethanol or hexadecyl alcohol, a glycol such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agents and other pharmaceutical adjuvants.

Oils, which can be used in parenteral formulations in some embodiments, include petroleum, animal oils, vegetable oils, or synthetic oils. Specific examples of oils include peanut, soybean, sesame, cottonseed, corn, olive, petrolatum, and mineral oil. Suitable fatty acids for use in parenteral formulations include oleic acid, stearic acid, and isostearic acid. Ethyl oleate and isopropyl myristate are examples of suitable fatty acid esters.

Suitable soaps for use in certain embodiments of parenteral formulations include fatty alkali metal, ammonium, and triethanolamine salts, and suitable detergents include (a) cationic detergents such as, for example, dimethyl dialkyl ammonium halides. and alkyl pyridinium halides, (b) anionic detergents such as, for example, alkyl, aryl, and olefin sulfonates, alky, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

In some embodiments, the parenteral formulations will contain, for example, from about 0.5% to about 25% by weight of the therapeutic agent in solution. Preservatives and buffers may be used. In order to minimize or eliminate irritation at the site of injection, such compositions may contain one or more nonionic surfactants having, for example, a hydrophile-lipophile balance (HLB) of from about 12 to about 17. The quantity of surfactant in such formulations will typically range, for example, from about 5% to about 15% by weight. Suitable surfactants include polyethylene glycol sorbitan fatty acid esters, such as sorbitan monooleate and high molecular weight adducts of ethylene oxide with a hydrophobic base, formed by the condensation of propylene oxide with propylene glycol. The parenteral formulations can be presented in unit-dose or multi-dose sealed containers, such as ampoules or vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind previously described.

In certain embodiments, injectable formulations are provided herein. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Toissel, 4th ed, pages 622-630 (1986)).

In some embodiments, topical formulations are provided herein. Topical formulations, including those that are useful for transdermal drug release, are suitable in the context of certain embodiments provided herein for application to skin. In some embodiments, the therapeutic agent alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

In certain embodiments, the therapeutic agents provided herein can be formulated as inclusion complexes, such as cyclodextrin inclusion complexes, or liposomes. Liposomes can serve to target the therapeutic agents to a particular tissue. Liposomes also can be used to increase the half-life of the therapeutic agents. Many methods are available for preparing liposomes, as described in, for example, Szoka et al., Ann. Rev. Biophys. Bioeng., 9, 467 (1980) and U.S. Pat. Nos. 4,235,871, 4,501,728, 4,837,028, and 5,019,369.

In some embodiments, the therapeutic agents provided herein are formulated in time-released, delayed release, or sustained release delivery systems such that the delivery of the composition occurs prior to, and with sufficient time to cause, sensitization of the site to be treated. Such systems can avoid repeated administrations of the therapeutic agent, thereby increasing convenience to the subject and the physician, and may be particularly suitable for certain composition embodiments provided herein. In one embodiment, the compositions of the invention are formulated such that they are suitable for extended-release of the circRNA contained therein. Such extended-release compositions may be conveniently administered to a subject at extended dosing intervals. For example, in one embodiment, the compositions of the present invention are administered to a subject twice day, daily or every other day. In an embodiment, the compositions of the present invention are administered to a subject twice a week, once a week, every ten days, every two weeks, every three weeks, every four weeks, once a month, every six weeks, every eight weeks, every three months, every four months, every six months, every eight months, every nine months or annually.

In some embodiments, a protein encoded by an inventive polynucleotide is produced by a target cell for sustained amounts of time. For example, the protein may be produced for more than one hour, more than four, more than six, more than 12, more than 24, more than 48 hours, or more than 72 hours after administration. In some embodiments the polypeptide is expressed at a peak level about six hours after administration. In some embodiments the expression of the polypeptide is sustained at least at a therapeutic level. In some embodiments the polypeptide is expressed at least at a therapeutic level for more than one, more than four, more than six, more than 12, more than 24, more than 48, or more than 72 hours after administration. In some embodiments, the polypeptide is detectable at a therapeutic level in patient serum or tissue (e.g., liver or lung). In some embodiments, the level of detectable polypeptide is from continuous expression from the circRNA composition over periods of time of more than one, more than four, more than six, more than 12, more than 24, more than 48, or more than 72 hours after administration.

In certain embodiments, a protein encoded by an inventive polynucleotide is produced at levels above normal physiological levels. The level of protein may be increased as compared to a control. In some embodiments, the control is the baseline physiological level of the polypeptide in a normal individual or in a population of normal individuals. In other embodiments, the control is the baseline physiological level of the polypeptide in an individual having a deficiency in the relevant protein or polypeptide or in a population of individuals having a deficiency in the relevant protein or polypeptide. In some embodiments the control can be the normal level of the relevant protein or polypeptide in the individual to whom the composition is administered. In other embodiments the control is the expression level of the polypeptide upon other therapeutic intervention, e.g., upon direct injection of the corresponding polypeptide, at one or more comparable time points.

In certain embodiments, the levels of a protein encoded by an inventive polynucleotide are detectable at 3 days, 4 days, 5 days, or 1 week or more after administration. Increased levels of secreted protein may be observed in the serum and/or in a tissue (e.g., liver or lung).

In some embodiments, the method yields a sustained circulation half-life of a protein encoded by an inventive polynucleotide. For example, the protein may be detected for hours or days longer than the half-life observed via subcutaneous injection of the protein or mRNA encoding the protein. In some embodiments, the half-life of the protein is 1 day, 2 days, 3 days, 4 days, 5 days, or 1 week or more.

Many types of release delivery systems are available and known to those of ordinary skill in the art. They include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; hydrogel release systems; sylastic systems; peptide based systems: wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

In some embodiments, the therapeutic agent can be conjugated either directly or indirectly through a linking moiety to a targeting moiety. Methods for conjugating therapeutic agents to targeting moieties is known in the art. See, for instance, Wadwa et al., J, Drug Targeting 3:111 (1995) and U.S. Pat. No. 5,087,616.

In some embodiments, the therapeutic agents provided herein are formulated into a depot form, such that the manner in which the therapeutic agent is released into the body to which it is administered is controlled with respect to time and location within the body (see, for example, U.S. Pat. No. 4,450,150). Depot forms of therapeutic agents can be, for example, an implantable composition comprising the therapeutic agents and a porous or non-porous material, such as a polymer, wherein the therapeutic agents are encapsulated by or diffused throughout the material and/or degradation of the non-porous material. The depot is then implanted into the desired location within the body and the therapeutic agents are released from the implant at a predetermined rate.

### 9. Therapeutic Methods

In certain aspects, provided herein is a method of treating and/or preventing a condition, e.g., cancer.

In certain embodiments, the therapeutic agents provided herein are coadministered with one or more additional therapeutic agents (e.g., in the same pharmaceutical composition or in separate pharmaceutical compositions). In some embodiments, the therapeutic agent provided herein can be administered first and the one or more additional therapeutic agents can be administered second, or vice versa. Alternatively, the therapeutic agent provided herein and the one or more additional therapeutic agents can be administered simultaneously.

In some embodiments, the subject is a mammal. In some embodiments, the mammal referred to herein can be any mammal, including, but not limited to, mammals of the order Rodentia, such as mice and hamsters, or mammals of the order Logomorpha, such as rabbits. The mammals may be from the order Carnivora, including Felines (cats) and Canines (dogs). The mammals may be from the order Artiodactyla, including Bovines (cows) and Swines (pigs), or of the order Perssodactyla, including Equines (horses). The mammals may be of the order Primates, Ceboids, or Simoids (monkeys) or of the order Anthropoids (humans and apes). Preferably, the mammal is a human.

### 10. Sequences

TABLE 17
IRES sequences.

| SEQ | | |
|---|---|---|
| ID NO | IRES | Sequence |
| 1 | EMCV-A | cccccctctccctccccccctaacgttactggccgaagccgcttggaataaggccggtgtgcgttt |
| | | gtctatatgttattttccaccatattgccgtcttttggcaatgtgagggcccggaaacctggccctgtct |
| | | tcttgacgagcattcctaggggtctttcccctctcgccaaaggaatgcaaggtctgttgaatgtcgtg |
| | | aaggaagcagttcctctggaagcttcttgaagacaaacaacgtctgtagcgaccctttgcaggcag |
| | | cggaaccccccacctggcgacaggtgcctctgcggccaaaagccacgtgtataagatacacctg |
| | | caaaggcggcacaaccccagtgccacgttgtgagttggatagttgtggaaagagtcaaatggctc |
| | | tcctcaagcgtattcaacaaggggctgaaggatgcccagaaggtaccccattgtatgggatctgat |
| | | ctggggcctcggtgcacatgctttacatgtgtttagtcgaggttaaaaaacgtctaggccccccgaa |
| | | ccacggggacgtggttttcctttgaaaaacacgatgataatatggccacaacc |
| 2 | EMCV-B | ctccccctccccccccttactatactggccgaagccacttggaataaggccggtgtgcgtttgtcta |
| | | catgctattttctaccgcattaccgtcttatggtaatgtgagggtccagaacctgaccctgtcttcttga |
| | | cgaacactcctaggggtctttcccctctcgacaaaggagtgtaaggtctgttgaatgtcgtgaagga |
| | | agcagttcctctggaagcttcttaaagacaaacaacgtctgtagcgaccctttgcaggcagcggaa |
| | | ccccccacctggtgacaggtgcctctgcggccaaaagccacgtgtataagatacacctgcaaag |
| | | gcggcacaaccccagtgccacgttgtgagttggatagttgtggaaagagtcaaatggctctcctca |
| | | agcgtattcaacaaggggctgaaggatgcccagaaggtaccccattgtatgggatctgatctggg |
| | | gcctcggtgcacgtgctttacacgtgttgagtcgaggtgaaaaaacgtctaggccccccgaacca |
| | | cggggacgtggttttcctttgaaaaccacgattacaat |
| 3 | EMCV-Bf | ttgccagtctgctcgatatcgcaggctgggtccgtgactacccactccccctttcaacgtgaaggct |
| | | acgatagtgccagggcgggtactgccgtaagtgccaccccaaacaacaacaacaaaacaaactc |
| | | cccctccccccccttactatactggccgaagccacttggaataaggccggtgtgcgtttgtctacat |
| | | gctattttctaccgcattaccgtcttatggtaatgtgagggtccagaacctgaccctgtcttcttgacg |
| | | aacactcctaggggtctttcccctctcgacaaaggagtgtaaggtctgttgaatgtcgtgaaggaag |
| | | cagttcctctggaagcttcttaaagacaaacaacgtctgtagcgaccctttgcaggcagcggaacc |
| | | ccccacctggtgacaggtgcctctgcggccaaaagccacgtgtataagatacacctgcaaaggc |
| | | ggcacaaccccagtgccacgttgtgagttggatagttgtggaaagagtcaaatggctctcctcaag |
| | | cgtattcaacaaggggctgaaggatgcccagaaggtaccccattgtatgggatctgatctggggc |
| | | ctcggtgcacgtgctttacacgtgttgagtcgaggtgaaaaaacgtctaggccccccgaaccacg |
| | | gggacgtggttttcctttgaaaaccacgattacaat |
| 4 | EMCV-Cf | ttgccagtctgctcgatatcgcaggctgggtccgtgactacccactccccctttcaacgtgaaggct |
| | | acgatagtgccagggcgggtactgccgtaagtgccaccccaaaacaacaacaaccccccctctc |
| | | cctccTccccccctaacgttactggccgaagccgcttggaataaggccggtgtgcgtttgtctatat |
| | | gttattttccaccatattgccgtcttttggcaatgtgagggcccggaaacctggccctgtcttcttgac |
| | | gagcattcctaggggtctttcccctctcgccaaaggaatgcaaggtctgttgaatgtcgtgaaggaa |
| | | gcagttcctctggaagcttcttgaagacaaacaacgtctgtagcgaccctttgcaggcagcggaac |
| | | cccccacctggcgacaggtgcctctgcggccaaaagccacgtgtataagatacacctgcaaagg |
| | | cggcacaaccccagtgccacgttgtgagttggatagttgtggaaagagtcaaatggctctcctcaa |
| | | gcgtattcaacaaggggctgaaggatgcccagaaggtaccccattgtatgggatctgatctgggg |
| | | cctcggtgcacatgctttacatgtgtttagtcgaggttaaaaaacgtctaggccccccgaaccacgg |
| | | ggacgtggttttcctttgaaaaacacgatgataat |
| 5 | EMCV pEC9 | ccccccccctaacgttactggccgaagccgcttggaataaggccggtgtgcgtttgtctatatgttat |
| | | tttccaccatattgccgtcttttggcaatgtgagggcccggaaacctggccctgtcttcttgacgagc |
| | | attcctaggggtctttcccctctcgccaaaggaatgcaaggtctgttgaatgtcgtgaaggaagcag |
| | | ttcctctggaagcttcttgaagacaaacaacgtctgtagcgaccctttgcaggcagcggaaccccc |
| | | cacctggcgacaggtgcctctgcggccaaaagccacgtgtataagatacacctgcaaaggcggc |
| | | acaaccccagtgccacgttgtgagttggatagttgtggaaagagtcaaatggctctcctcaagcgt |
| | | attcaacaaggggctgaaggatgcccagaaggtaccccattgtatgggatctgatctggggcctc |
| | | ggtgcacatgctttacatgtgtttagtcgaggttaaaaaacgtctaggccccccgaaccacgggga |
| | | cgtggttttcctttgaaaaacacgatgataat |
| 6 | Picobirnavirus | gtaaattaaatgctatttacaaaatttaaacagaaaggagagatgttatgaaccggttttacaaggttt |
| | | catacatcgaaaatagcactacctggggcagccgacacactaacatcgtctgtttaaccagaagtg |
| | | ttactgaaaggaggttattta |
| 7 | HCV QC64 | acctgcccctaataggggcgacactccgccatgaatcactcccctgtgaggaactactgtcttcac |
| | | gcagaaagcgtctagccatggcgttagtatgagtgtcgtacagcctccaggcccccccctcccgg |
| | | gagagccatagtggtctgcggaaccggtgagtacaccggaattgccgggaagactgggtcctttc |
| | | ttggataaacccactctatgcccggacatttgggcgtgcccccgcaagactgctagccgagtagc |
| | | gttgggttgcgaaaggccttgtggtactgcctgatagggtgcttgcgagtgccccgggaggtctcg |
| | | tagaccgtgcatc |
| 8 | Human Cosavirus | ctacaagctttgtgtaaacaaacttttgtttggcttttctcaagcttctctcacatcaggccccaaagat |
| | E/D | gtcctgaaggtaccccgtgtatctgaggatgagcaccatcgactacccggacctgcaaaattttgc |
| | | aaacgcatgtggtatcccagccccctcctctcggggagggggctttgctcactcagcacaggatct |
| | | gatcaggagatccacctccggtgctttacaccggggcgtggatttaaaaattgcccaaggcctggc |
| | | gcacaacctaggggactaggttttccttatattttaaagctgtcaat |
| 9 | Human Cosavirus | gtcttaggacgacgcatgtggtatcccagcccccgcctacattggcgggggcttttgaagcacca |
| | F | gacactggatctgatcaggaggagggtagctgctttacagcccctcttaaaaattgcccaaggtcc |
| | | ggccacccaacctaggggactaggttttccttttatttttaaattgtcatt |
| 10 | Human Cosavirus | acatgggggagactgcatgtggcagtcttgaaacgtgtggtttgacgtctaccttatatggcagtgg |
| | JMY | gtggagtactgcaaagatgtcaccgtgctttacacggtttttgaaccccacaccggctgtttgacgct |
| | | cgtagggcagcaggtttattttcattaaaattcttactttctagctgcatgagttctattcatgcagacgg |
| | | agtgatactcccgttccttcttggacaggttgcctccacgccctttgtggatcttaaggtgaccaagtc |
| | | actggtgttggaggtgaagatagagagtcctcttgggaatgtcatgtggctgtgccaggggttgta |
| | | gcgatgccattcgtgtgtgcggatttcctctcgtggtgacacgagcctcacaggccaaaagccccg |
| | | tccgaaaggacccgaatggtggagtgaccctgactcccccctgcatagttttgtgattaggaacttg |
| | | aggaatttctgtcataaatctctatcacatcaggccccaaagatgtcctgaaggtaccctgtgtatctg |
| | | aggatgagcaccaccgactacccggacttgcattagcagacacatgtggttgcccagccccacct |
| | | cttcagaggtggggctttgctcactcagcacaggatctgatcaggagccccgctcgtgtgctttaca |
| | | ctcgacgcggggttaaaaattgcccaaggcctggcacaacaacctaggggactaggttttcctattt |
| | | ttgtaaattatgtcaat |
| 11 | Rhinovirus | gtgacaatcagccagattgttaacggtcaagcacttctgtttccccggtacccttgtatacgcttcacc |
| | NAT001 | cgaggcgaaaagtgaggttatcgttatccgcaaagtgcctacgagaagcctagtagcacttttgaa |
| | | gcctatggctggtcgctcaactgtttacccagcagtagacctggcagatgaggctagatgttcccc |
| | | accagcgatggtgatctagcctgcgtggctgcctgcacactctattgagtgtgaagccagaaagtg |
| | | gacaaggtgtgaagagcctattgtgctcactttgagtcctccggcccctgaatgtggctaatcctaa |
| | | ccccgtagctgttgcatgtaatccaacatgtctgcagtcgtaatgggcaactatgggatggaaccaa |
| | | ctactttgggtgtccgtgtttcttgtttttctttatgcttgcttatggtgacaactgtagttattacatttgtta |
| | | cc |
| 12 | HRV14 | ttaaaacagcggatgggtatcccaccattcgacccattgggtgtagtactctggtactatgtacctttg |
| | | tacgcctgtttctccccaaccacccttccttaaaattcccacccatgaaacgttagaagcttgacatta |
| | | aagtacaataggtggcgccatatccaatggtgtctatgtacaagcacttctgtttcccaggagcgag |
| | | gtataggctgtacccactgccaaaagcctttaaccgttatccgccaaccaactacgtaacagttagt |
| | | accatcttgttcttgactggacgttcgatcaggtggattttccctccactagtttggtcgatgaggcta |
| | | ggaattccccacgggtgaccgtgtcctagcctgcgtggcggccaacccagcttatgctgggacgc |
| | | ccttttaaggacatggtgtgaagactcgcatgtgcttggttgtgagtcctccggcccctgaatgcgg |
| | | ctaaccttaaccctagagccttatgccacgatccagtggttgtaaggtcgtaatgagcaattccggg |
| | | acgggaccgactactttgggtgtccgtgtttctcatttttcttcatattgtcttatggtcacagcatatata |
| | | tacatatactgtgatc |
| 13 | HRV89 | ttaaaactgggagtgggttgttcccactcactccacccatgcggtgttgtactctgttattacggtaac |
| | | tttgtacgccagtttttcccacccttccccataatgtaacttagaagtttgtacaatatgaccaataggt |
| | | gacaatcatccagactgtcaaaggtcaagcacttctgtttccccggtcaatgaggatatgctttaccc |
| | | aaggcaaaaaccttagagatcgttatccccacactgcctacacagagcccagtaccatttttgatat |
| | | aattgggttggtcgctccctgcaaacccagcagtagacctggcagatgaggctggacattcccca |
| | | ctggcgacagtggtccagcctgcgtggctgcctgctcacccttcttgggtgagaagcctaattattg |
| | | acaaggtgtgaagagccgcgtgtgctcagtgtgcttcctccggcccctgaatgtggctaaccttaa |
| | | ccctgcagccgttgcccataatccaatgggtttgcggtcgtaatgcgtaagtgcgggatgggacca |
| | | actactttgggtgtccgtgtttcctgtttttcttttgattgcattttatggtgacaatttatagtgtatagattg |
| | | tcatc |
| 14 | HRVC-02 | ttaaaactgggtacaggttgttcccacctgtatcacccacgtggtgtggtgctcttgtattccggtaca |
| | | cttgcacgccagtttgccacccctcacccgtcgtaacttagaagctaacaactcgaccaacaggcg |
| | | gtggtaaaccataccacttacggtcaagcactcctgtttccccggtatgcgaggaatagactcctac |
| | | agggttgaagcctcaagtatcgttatccgcattggtactacgcaaagcttagtagtgccttgaaagtc |
| | | ccttggttggtcgctccgctagtttcccctagtagacctggcagatgaggcaggacactccccact |
| | | ggcgacagtggtcctgcctgcgtggctgcctgcgcacccttaggggtgcgaagccaagtgacag |
| | | acaaggtgtgaagagccccgtgtgctaccaatgagtcctccggcccctgaatgcggctaatccaa |
| | | ccccacagctattgcacacaagccagtgtgtatgtagtcgtaatgagcaattgtgggacggaaccg |
| | | actactttgggtgtccgtgtttccttttattcttatcattctgcttatggtgacaatactgtgaaatagtgtt |
| | | gttacc |
| 15 | HRV-A21 | taaaactggatccaggttgttcccacctggatctcctattgggagttgtactctattattccggtaatttt |
| | | gtacgccagttttatcttccccctccccaattgtaacttagaaggttatcaatacgaccaataggtggt |
| | | agttagccaaactaccaaaggtcaagcacttctgtttccccggtcaaagttgatatgctccaacagg |
| | | gcaaaaacaactgagatcgttatccgcaaagtgcctacgcaaagcctagtaacacctttgaagattt |
| | | atggttggtcgttccgctatttcccatagtagacctggcagatgaggctagaaatcccccactggcg |
| | | acagtgctctagcctgcgtggctgcctgcgcaccccttgggtgcgaagccatacattggacaagg |
| | | tgtgaagagccccgtgtgctcactttgagtcctccggcccctgaatgtggctaaccttaaccctgca |
| | | gctagtgcatgtaatccaacatgttgctagtcgtaatgagtaattgcgggacgggaccaactactttg |
| | | ggtgtccgtgtttcactttttccttttaatattgcttatggtgacaatatatatagctatatatattgacacc |
| 16 | Salivirus A SH1 | ttcccctgcaaccattacgcttactcgcatgtgcattgagtggtgcatgtgttgaacaaacagctaca |
| | | ctcacatgggggcgggttttcccgccctacggcttctcgcgaggcccacccctcccctttctcccat |
| | | aactacagtgctttggtaggtaagcatcctgatcccccgcggaagctgctcacgtggcaactgtgg |
| | | ggacccagacaggttatcaaaggcacccggtctttccgccttcaggagtatccctgctagcgaatt |
| | | ctagtagggctctgcttggtgccaacctcccccaaatgcgcgctgcgggagtgctcttccccaact |
| | | caccctagtatcctctcatgtgtgtgcttggtcagcatatctgagacgatgttccgctgtcccagacc |
| | | agtccagtaatggacgggccagtgtgcgtagtcgtcttccggcttgtccggcgcatgtttggtgaac |
| | | cggtggggtaaggttggtgtgcccaacgcccgtactcaggggatacctcaaggcacccaggaat |
| | | gccagggaggtaccccgcttcacagcgggatctgaccctggggtaaatgtctgcggggggtcttc |
| | | ttggcccacttctcagtacttttcagg |
| 17 | Salivirus FHB | acatggggggtctgcggacggcttcggcccacccgcgacaagaatgccgtcatctgtcctcatta |
| | | cccgtattccttcccttcccccgcaaccaccacgcttactcgcgcacgtgttgagtggcacgtgcgt |
| | | tgtccaaacagctacacccacacccttcggggcgggtttgtcccgccctcgggttcctcgcggaa |
| | | cccccccctccctctctctctttctatccgccctcacttcccataactacagtgctttggtaggtgagc |
| | | accctgaccccccgcggaagctgctaacgtggcaactgtggggatccaggcaggttatcaaagg |
| | | cacccggtctttccgccttcaggagtatctctgccggtgaattccggtagggctctgcttggtgcca |
| | | acctcccccaaatgcgcgctgcgggagtgctcttccccaactcatcttagtaacctctcatgtgtgtg |
| | | cttggtcagcatatctgaggcgacgttccgctgtcccagaccagtccagcaatggacgggccagt |
| | | gtgcgtagtcgctttccggttttccggcgcatgtttggcgaaacgctgaggtaaggttggtgtgccc |
| | | aacgcccgtaatttggtgatacctcaagaccacccaggaatgccagggaggtaccccacttcggt |
| | | gggatctgaccctgggctaattgtctacggtggttcttcttgcttccacttctcttttttctggcatg |
| 18 | Salivirus NG-J1 | tatggcaggcgggcttgtggacggcttcggcccacccacagcaagaatgccatcatctgtcctca |
| | | cccccaattttcccttttcttcccctgcaaccattacgcttactcgcatgtgcattgagtggtgcatgtgt |
| | | tgaacaaacagctacactcacatgggggcgggttttcccgccctacggcctctcgcgaggcccac |
| | | cccttccctccccttataactacagtgctttggtaggtaagcatcctgatcccccgcggaagctgctc |
| | | acgtggcaactgtggggacccagacaggttatcaaaggcacccggtctttccgccttcaggagtat |
| | | ccctactagtgaattctagcggggctctgcttggtgccaacctcccccaaatgcgcgctgcgggag |
| | | tgctcttccccaactcaccctagtatcctctcatgtgtgtgcttggtcagcatatctgagacgatgttcc |
| | | gctgtcccagaccagtccagtaatggacgggccagtgcgtgtagtcgtcttccggcttgtccggg |
| | | gcatgtttggtgaaccggtggggtaaggttggtgtgcccaacgcccgtactttggtgacacctcaa |
| | | gaccacccaggaatgccagggaggtaccccacctcacggtgggatctgaccctgggctaattgt |
| | | ctacggtggttcttcttgcttccacttctttcttctgttcacg |
| 19 | Human | tttgaaaggggtctcctagagagcttggccgtcgggccttataccccgacttgctgagtttctctagg |
| | Parechovirus 1 | agagcccttttcccagccctgaggcggctggtcaataaaagcctcaaacgtaactaacacctaaga |
| | | agatcatgtaaaccctatgcctggtctccactattcgaaggcaacttgcaataagaagagtgggatc |
| | | aagacgcttaaagcatagagacagttttcttttctaacccacatttgtgtggggtggcagatggcgtg |
| | | ccataactctaatagtgagataccacgcttgtggaccttatgctcacacagccatcctctagtaagttt |
| | | gtgagacgtctggtgacgtgtgggaacttattggaaacaacattttgctgcaaagcatcctactgcc |
| | | agcggaaaaacacctggtaacaggtgcctctggggccaaaagccaaggtttaacagaccctttag |
| | | gattggttctaaacctgagatgttgtggaagatatttagtacctgctgatctggtagttatgcaaacact |
| | | agttgtaaggcccatgaaggatgcccagaaggtacccgtaggtaacaagtgacactatggatctg |
| | | atttggggccagatacctctatcttggtgatctggttaaaaaacatctaatgggccaaacccggggg |
| | | ggatccccggtttcctcttattctatcaatgccact |
| 20 | Crohivirus B | gtataagagacaggtgtttgccttgtcttcggactggcatcttgggaccaaccccccttttccccagc |
| | | catgggttaaatggcaataaaggacgtaacaactttgtaaccattaagctttgtaattttgtaaccact |
| | | aagctttgtgcacataatgtaaccatcaagcttgttagtcccagcaggaggtttgcatgcttgtagcc |
| | | gaaatggggctcgaccccccatagtaggatacttgattttgcattccattgtggacctgcaaactcta |
| | | cacatagaggctttgtcttgcatctaaacacctgagtacagtgtgtacctagaccctatagtacggga |
| | | ggaccgtttgtttcctcaataaccctacataataggctaggtgggcatgcccaatttgcaagatccca |
| | | gactgggggtcggtctgggcagggttagatccctgttagctactgcctgatagggtggtgctcaac |
| | | catgtgtagtttaaattgagctgttcatatacc |
| 21 | Yc-3 | actgaagatcctacagtaactactgccccaatgaacgccacagatgggtctgctgatgactacctat |
| | | cttagtgctagttgaggtttgaagtgagccggtttttagaagaaccagtttctgaacattatcatcccc |
| | | agcatctattctatacgcacaagatagatagtcatcagcagacacatctgtgctactgcttgatagag |
| | | ttgcggctggtcaacttagattggtataaccagttgagtggcaa |
| 22 | Rosavirus M-7 | tatgcatcactggacggcctaacctcggtcgtggcttcttgccgatttcagcgctaccaggctttctg |
| | | gtctcgccaggcgttgattagtaggtgcactgtctaagtgaagacagcagtgctctctgtgaaaagt |
| | | tgatgacactcttcaggtttgtagcgatcactcaaggctagcggatttccccgtgtggtaacacacg |
| | | cctctaggcccagaaggcacggtgttgacagcaccccttgagtggctggtcttccccaccagcac |
| | | ctgatttgtggattcttcctagtaacggacaagcatggctgctcttaagcattcagtgcgtccggggc |
| | | tgaaggatgcccagaaggtacccgcaggtaacgataagctcactgtggatctgatctggggctgc |
| | | gggctgggtgtctttccacccagccaaaacccgtaaaacggtagtcgcagttaaaaaacgtctag |
| | | gccccacccccccagggatggggggttcccttaaaccctcacaagttcaac |
| 23 | Shanbavirus A | tgaaaagggggcgcagggtggtggtggttactaaatacccaccatcgccctgcacttcccttttcc |
| | | cctgtggctcagggtcacttagccccctctttgggttaccagtagttttctacccctgggcacagggt |
| | | taactatgcaagacggaacaacaatctcttagtccccctcgccgatagtgggctcgacccccatgt |
| | | gtaggagtggataagggacggagtgagccgatacggggaagagtgtgcggtcacaccttaattc |
| | | catgagcgctgcgaagaaggaagctgtgaacaatggcgacctgaaccgtacacatggagctcca |
| | | caggcatggtactcgttagactacgcagcctggttgggagtgggtataccctgggtgagccgcca |
| | | gtgaatgggagttcactggttaacacacactgcctgatagggtcagggcctcctgtccccgccgta |
| | | atgaggtagaccatatgcc |
| 24 | Pasivirus A | gcggctggatattctggccgtgcaactgcttttgaccagtggctctgggtaacttagccaaagtgtc |
| | | cttctccctttccctattatatgttttatggctttgtctggtcttgtttagtttatatataagatcctttccgcc |
| | | gatatagacctcgacagtctagtgtaggaggattggtgatattaatttgccccagaagagtgaccgt |
| | | gacacatagaaaccatgagtacatgtgtatccgtggaggatcgcccgggactggattccatatccc |
| | | attgccatcccaacaagcggagggtatacccactatgtgcacgtctgcagtgggagtctgcagatt |
| | | tagtcatactgcctgatagggtgtgggcctgcactctggggtactcaggctgtttatataat |
| 25 | Pasivirus A 2 | gctggactttctggctgcgcaactgcttttaaccagtggctctgggttacttagccaaaaccccctttc |
| | | cccgtaccctagtttgtgtgtgtattattattttgttgttgttttgtaaatttttatataagatcctttccgccg |
| | | atatagacctcgacagtctagtgtaggaggattggtgatattaatatgccccagaagagtgaccgtg |
| | | acacatagaaaccatgagtacatgtgtatccgtggaggatcgcccgggactggattccatatccca |
| | | ttgccatcccaacaaacggagggtatacccgctatgtgcgcgtctacagtgggaatctgtagattta |
| | | gtcatactgcctgatagggtgtgggcctgcactctggggtactcaggctgtttatataat |
| 26 | Echo virus E14 | ttaaaacagcctgtgggttgttcccatccacagggcccactgggcgccagcactctggtattgcgg |
| | | taccttagtgcgcctgttttatatacccgtcccccaaacgtaacttagacgcatgtcaacgaagacca |
| | | atagtaagcgcagcacaccagctgtgttccggtcaagcacttctgttaccccggaccgagtatcaa |
| | | taagctactcacgtggctgaaggagaaaacgttcgttacccgaccaattacttcaagaaacctagta |
| | | acaccatgaaggttgcgcagtgtttcgctccgcacaaccccagtgtagatcaggtcgatgagtcac |
| | | cgcattccccacgggtgaccgtggcggtggctgcgctggcggcctgcccatggggaaacccat |
| | | gggacgcttcaatactgacatggtgcgaagagtctattgagctaattggtagtcctccggcccctga |
| | | atgcggctaatcctaactgcggagcagatacccacacaccagtgggcagtctgtcgtaacgggca |
| | | actctgcagcggaaccgactactttgggtgtccgtgtttctctttatccttatactggctgcttatggtg |
| | | acaattgagagattgttaccatatagctattggattggccatccggtgacaaatagagcaattgtgtat |
| | | ttgtttgttggtttcgtgccattaaattacaaggttctaaacacccttaatcttattatagcattcaacaca |
| | | acaaa |
| 27 | Human | gtacattagatgcgtcatctgcaactttagtcaataaattacctccaatgtcattaccaacattccctac |
| | Parechovirus 5 | cttttcactaacacctaagacaacaagtacctatgcctggtctccactattcgaaggcaacttgcaat |
| | | aagaagagtggaattaagacgcttaaagcatagagctagttatcttttctaacccacaaagttttgtg |
| | | gggtggcagatggcgtgccataactctattagtgagataccatgcttgtggatcttatgctcacaca |
| | | gccatcctctagtaagttgataaggtgtctggtgatatgtgggaactcacatgaaccattaatttaccg |
| | | taaggtatcctatagccagcggaatcacatctggtgacagatgcctctggggccgaaagccaagg |
| | | tttaacagaccctataggattggtttcaaaacctgaattgatgtggattgtgtatagtacctgttgatct |
| | | ggtaacagtgtcaacactagttgtaaggcccacgaaggatgcccagaaggtacccgtaggtaaca |
| | | agtgacactatggatctgatctggggccagctacctctatcatggtgagttggttaaaaaacgtctag |
| | | tgggccaaacccaggggggatccctggtttccttttacctaatcaaagccact |
| 28 | Aichi Virus | tttgaaaagggggtgggggggcctcggccccctcaccctcttttccggtggtctggtcccggacc |
| | | accgttactccattcagcttcttcggaacctgttcggaggaattaaacgggcacccatactcccccc |
| | | accccccttttgtaactaagtatgtgtgctcgtgatcttgactcccacggaacggaccgatccgttgg |
| | | tgaacaaacagctaggtccacatcctcccttcccctgggagggcccccgccctcccacatcctcc |
| | | ccccagcctgacgtatcacaggctgtgtgaagcccccgcgaaagctgctcacgtggcaattgtgg |
| | | gtccccccttcatcaagacaccaggtctttcctccttaaggctagccccggcgtgtgaattcacgttg |
| | | ggcaactagtggtgtcactgtgcgctcccaatctcggccgcggagtgctgttccccaagccaaac |
| | | ccctggcccttcactatgtgcctggcaagcatatctgagaaggtgttccgctgtggctgccaacctg |
| | | gtgacaggtgccccagtgtgcgtaaccttcttccgtctccggacggtagtgattggttaagatttggt |
| | | gtaaggttcatgtgccaacgccctgtgcgggatgaaacctctactgccctaggaatgccaggcag |
| | | gtaccccacctccgggtgggatctgagcctgggctaattgtctacgggtagtttcatttccaatccttt |
| | | tatgtcggagtc |
| 29 | Hepatitis A Virus | ttcaagaggggtctccggagttttccggaacccctcttggaagtccatggtgaggggacttgatac |
| | HA16 | ctcaccgccgtttgcctaggctataggctaaatttccctttccctgtccttcccctatttccttttgttttgt |
| | | ttgtaaatattaattcctgcaggttcagggttctttaatctgtttctctataagaacactcaatttttcacgc |
| | | tttctgtctcctttcttccagggctctccccttgccctaggctctggccgttgcgcccggcggggtca |
| | | actccatgattagcatggagctgtaggagtctaaattggggacgcagatgtttgggacgtcgccttg |
| | | cagtgttaacttggctttcatgaacctctttgatcttccacaaggggtaggctacgggtgaaacctctt |
| | | aggctaatacttcaatgaagagatgccttggatagggtaacagcggcggatattggtgagttgttaa |
| | | gacaaaaaccattcaacgccggaggactggctctcatccagtggatgcattgagggaattgattgt |
| | | cagggctgtctctaggtttaatctcagacctctctgtgcttagggcaaacactatttggccttaaatgg |
| | | gatcctgtgagagggggtccctccattgacagctggactgttctttggggccttatgtggtgtttgcc |
| | | tctgaggtactcaggggcatttaggtttttcctcattcttaaataata |
| 30 | Phopivirus | gggagtaaacctcaccaccgtttgccgtggtttacggctacctatttttggatgtaaatattaattcctg |
| | | caggttcaggtctcttgaattatgtccacgctagtggcactctcttacccataagtgacgccttagcg |
| | | gaacctttctacacttgatgtggttaggggttacattatttccctgggccttctttggccctttttcccctg |
| | | cactatcattctttcttccgggctctcagcatgccaatgttccgaccggtgcgcccgccggggttaa |
| | | ctccatggttagcatggagctgtaggccctaaaagtgctgacactggaactggactattgaagcat |
| | | acactgttaactgaaacatgtaactccaatcgatcttctacaaggggtaggctacgggtgaaacccc |
| | | ttaggttaatactcatattgagagatacttctgataggttaaggttgctggataatggtgagtttaacga |
| | | caaaaaccattcaacagctgtgggccaacctcatcaggtagatgcttttggagccaagtgcgtagg |
| | | ggtgtgtgtggaaatgcttcagtggaaggtgccctcccgaaaggtcgtaggggtaatcaggggca |
| | | gttaggtttccacaattacaatttgaa |
| 31 | CVA10 | gctcttccgatctgggttgttcccacccacagggcccactgggcgccagcactctgattccacgga |
| | | atctttgtgcgcctgttttacaacccttcccaatttgtaacgtagaagcaatacacactactgatcaata |
| | | gtaggcatggcgcgccagtcatgtcatgatcaagcacttctgttcccccggactgagtatcaataga |
| | | ctgctcacgcggttgaaggagaaaacgttcgttacccggctaactacttcgagaaacctagtagca |
| | | ccatggaagctgcggagtgtttcgctcagcactttccccgtgtagatcaggtcgatgagtcactgca |
| | | atccccacgggcgaccgtggcagtggctgcgttggcggcctgcctatggggcaacccataggac |
| | | gctctaatgtggacatggtgcgaagagtctattgagctagttagtagtcctccggcccctgaatgcg |
| | | gctaatcctaactgcggagcacatgccttcaacccaggaggtggtgtgtcgtaacgggtaactctg |
| | | cagcggaaccgactactttgggtgtccgtgtttccttttatccttatattggctgcttatggtgacaatc |
| | | acggaattgttgccatatagctattggattggccatccggtgtctaacagagctattgtatacctatttg |
| | | ttggatttactcccctatcatacaaatctctgaacactttgtgctttatactgaacttaaacacacgaaa |
| 32 | Enterovirus C | ttaaaacagctctggggttgttcccaccccagaggcccacgtggcggccagtacaccggtaccac |
| | | ggtacccttgtacgcctgttttatactcccctccccgtaaactagaagcacgaaacacaagttcaata |
| | | gaagggggtacagaccagtaccaccacgaacaagcacttctgttcccccggtgaggtcacatag |
| | | actgtccccacggtcaaaagtgactgatccgttatccgctcacgtacttcggaaagcctagtaccac |
| | | cttggaatctacgatgcgttgcgctcagcactcgaccccggagtgtagcttaggctgatgagtctg |
| | | gacgttccccactggtgacagtggtccaggctgcgttggcggcctacctgtggtccaaaaccaca |
| | | ggacgctagtagtgaacaaggtgtgaagagcccactgagctacctgagaatcctccggcccctg |
| | | aatgcggctaatcccaaccacggagcaggtaatcgcaaaccagcggtcagcctgtcgtaacgcg |
| | | taagtctgtggcggaaccgactactttgggtgtccgtgtttccttttatttttatggtggctgcttatggt |
| | | gacaatcatagattgttatcataaagcaaattggattggccatccggagtgagctaaactatctatttc |
| | | tctgagtgttggattcgtttcacccacattctgaacaatcagcctcattagtgttaccctgttaataaga |
| | | cgatatcatcacg |
| 33 | Enterovirus D | ttaaaacagctctggggttgttcccaccccagaggcccacgtggcggctagtactccggtacccc |
| | | ggtacccttgtacgcctgttttatactccctttcccaagtaactttagaagaaataaactaatgttcaac |
| | | aggagggggtacaaaccagtaccaccacgaacacacacttctgtttccccggtgaagttgcatag |
| | | actgtacccacggttgaaagcgatgaatccgttacccgcttaggtacttcgagaagcctagtatcat |
| | | cttggaatcttcgatgcgttgcgatcagcactctaccccgagtgtagcttgggtcgatgagtctgga |
| | | caccccacaccggcgacgtggtccaggctgcgttggcggcctacccatggctagcaccatggga |
| | | cgctagttgtgaacaaggtgcgaagagcctattgagctacctgagagtcctccggcccctgaatgc |
| | | ggctaatcccaaccacggagcaaatgctcacaatccagtgagtggtttgtcgtaatgcgcaagtct |
| | | gtggcggaaccgactactttgggtgtccgtgtttccttttatttttattatggctgcttatggtgacaatct |
| | | gagattgttatcatatagctattggattagccatccggtgatatcttgaaattttgccataactttttcaca |
| | | aatcctacaacattacactacactttctcttgaataattgagacaactcata |
| 34 | Enterovirus J | ttaaaatagcctcagggttgttcccaccctgagggcccacgtggtgtagtactctggtattacggtac |
| | | ctttgtacgcctattttatacccccttccccaagtaatttagaagcaagcacaaaccagttcagtagta |
| | | agcagtacaatccagtactgtaatgaacaagtacttctgttaccccggaagggtctatcggtaagct |
| | | gtacccacggctgaagaatgacctaccgttaaccggctacctacttcgagaagcctagtaatgccg |
| | | ttgaagttttattgacgttacgctcagcacactaccccgtgtgtagttttggctgatgagtcacggcac |
| | | tccccacgggcgaccgtggccgtggctgcgttggcggccaaccaaggagtgcaagctccttgga |
| | | cgtcatattacagacatggtgtgaagagcctattgagctaggtggtagtcctccggcccctgaatgc |
| | | ggctaatcctaactccggagcatatcggtgcgaaccagcacttggtgtgttgtaatacgtaagtctg |
| | | gagcggaaccgactactttgggtgtccgtgtttcctgttttaacttttatggctgcttatggtgacaattt |
| | | aacattgttaccatatagctgttgggttggccatccggattttgttataaaaccatttcctcgtgccttga |
| | | cctttaacacatttgtgaacttctttaaatcccttttattagtccttaaatactaaga |
| 35 | Human Pegivirus | aactgttgttgtagcaatgcgcatattgctacttcggtacgcctaattggtaggcgcccggccgacc |
| | 2 | ggccccgcaagggcctagtaggacgtgtgacaatgccatgagggatcatgacactggggtgag |
| | | cggaggcagcaccgaagtcgggtgaactcgactcccagtgcgaccacctggcttggtcgttcatg |
| | | gagggcatgcccacgggaacgctgatcgtgcaaagggatgggtccctgcactggtgccatgcg |
| | | cggcaccactccgtacagcctgatagggtggcggcgggcccccccagtgtgacgtccgtggag |
| | | cgcaac |
| 36 | GBV-C GT110 | tgacgtgggggggttgatTTTccccccccggcactgggtgcaagccccagaaaccgacgcct |
| | | atctaagtagacgcaatgactcggcgccgactcggcgaccggccaaaaggtggtggatgggtga |
| | | tgacagggttggtaggtcgtaaatcccggtcatcctggtagccactataggtgggtcttaagagaa |
| | | ggtcaagattcctcttacgcctgcggcgagaccgcgcacggtccacaggtgttggccctaccggt |
| | | gtgaataagggcccgacatcaggc |
| 37 | GBV-C K1737 | gacgtgggggggttgatccccccccTTTggcactgggtgcaagccccagaaaccgacgccta |
| | | tttaaacagacgttaagaaccggcgccgacccggcgaccggccaaaaggtggtggatgggtgat |
| | | gccagggttggtaggtcgtaaatcccggtcatcttggtagccactataggtgggtcttaagggttgg |
| | | ttaaggtccctctggcgcttgtggcgagaaagcgcacggtccacaggtgttggccctaccggtgt |
| | | gaataagggcccgacgtcaggctcgtcgttaaaccgagcccactacccacctgggcaaacaacg |
| | | cccacgtacggtccacgtcgcccttcaatgtctctcttgaccaataggcttagccggcgagttgaca |
| | | aggaccagtgggggctgggcggtaggggaaggacccctgccgctgcccttcccggtggagtg |
| | | ggaaatgc |
| 38 | GBV-C Iowa | tgacgtgggggggttgatccGccccccccggcactCggtgcaagccccataaaccgacgccta |
| | | tctaagtagacgcaatgactcggcgccgactcggcgaccggccaaaaggtggtggatgggtggt |
| | | gacagggttggtaggtcgtaaatcccggtcatcctggtagccactataggtgggtcttaagagaag |
| | | gtcaagactcctcttgtgcctgcggcgagaccgcgcacggtccacaggtgctggccctaccggtg |
| | | tgaataagggcccgacgtcaggctcgtcgttaaaccgagcccgtcacccacctgggcaaacgac |
| | | gcccacgtacggtccacgtcgcccttca |
| 39 | Pegivirus A 1220 | tgtagcaatgcgcatattgctacttcggtacgcctaattggtaggcgcccggccgaccggccccgc |
| | | aagggcctagtaggacgtgtgacaatgccatgcgggatcatgacactggggtgagcggaggca |
| | | gcaccgaagtcgggtgaactcgactcccagtgcgaccacctggcttggtcgttcatggagggcat |
| | | gcccacgggaacgctgatcgtgcaaagggatgggtccctgcactggtgccatgcgcggcacca |
| | | ctccgtacagcctgatagggtggcggcgggcccccccagtgtgacgtccgtggagcgcaac |
| 40 | Pasivirus A 3 | attttctggccgtgtagctgcttttgaccagtggctctgggttacttagccaaatcccccttccttcacc |
| | | cttttaaatttgatggtctgtgttgtttgttttgtcttgtctaaataatatataagatccttcccgccgataca |
| | | gacctcgacagtctggtgtaggagggttggtgttattaatttgccccagaagagtgaccgtgacac |
| | | atagaaaccatgagtacatgtgtatccgtggaggatcgcccgggactggattccatatcccattgcc |
| | | atcccaacaagcggagggtatacccactatgtgcgcgtttgcagtgggaatctgcaaatttagtcat |
| | | actgcctgatagggtgtgggcctgcactctggggtactcaggctgttcatataat |
| 41 | Sapelovirus | cccctccacccttaaggtggttgtatcccacataccccaccctcccttccaaagtggacggacaact |
| | | ggattttgactaacggcaagtctgaatggtatgatttggatacgtttaaacggcagtagcgtggcga |
| | | gctatggaaaaatcgcaattgtcgatagccatgttagtgacgcgcttcggcgtgctcctttggtgatt |
| | | cggcgactggttacaggagagtaggcagtgagctatgggcaaacctctacagtattacttagagg |
| | | gaatgtgcaattgagacttgacgagcgtctctttgagatgtggcgcatgctcttggcattaccatagt |
| | | gagcttccaggttgggaaacctggactgggcctatactacctgatagggtcgcggctggccgcct |
| | | gtaactagtatagtcagttgaaaccccccc |
| 42 | Rosavirus B | gtctctttagtgtctatgcttcagagagcggtgaactgacaccgttgcttcttgcacagcccttcgtgc |
| | | cggtctttccggttctcgacagcgttgggcatcatggctagttaggctaagatagtggatgatctagt |
| | | gaacagttttggattgtttggagttttgtagcgatgctagtagtgtgtgtggacctccccacgtggtaa |
| | | cacgtgccccacaggccaaaagccaaggtgttgaaagcacccctactagtcccagactcacccat |
| | | ctgggaactcctctcatgaaaaatcttagtaacttttgattcggctattcatcaacctctctagtcaagg |
| | | gctgaaggatgcccggaaggtacccgcaggtaacgataagctcactgtggatctgatccggggc |
| | | tttggtgcgaccgtctgtccggcgtagccagagttaaaaaacgtctaggcccttccaccccaaggg |
| | | attggggtttccccaatcatttgaaagttcact |
| 43 | Bakunsa Virus | ttttgaacgccacctcggagcgatatccggggaccccctcccctttttccttcctaccttcttcccaaa |
| | | tttccctcttcccttgttattttggtttggatttcctggacatgactcggacggatctatctcatttgctttgt |
| | | gtctgctccaccagtggcatggtcgaaagatcatcaacactggacgtgtactgtaatggccaaacg |
| | | tgcccacaggggaaaccatgccggtcgctgtagcggcgggtggacgtggtggacccctctccct |
| | | gctcataaactttgggtaggtgaagggttcaagcgacgcttgccgtgagggcgcatccggatggt |
| | | gggaaccaacaaactaggctgtaatggccgacctcaggtggatgagctagggctgctgcaccaa |
| | | aagggactcgattcgatatcccggcctggtagcctagtgcagtggactcgtagttgggaatctacg |
| | | actggcctagtacagggtgatagccccgtttcccacgcccacctgttgtagggacacccccccc |
| 44 | Tremovirus A | tttgaaagaggcctccggagtgtccggaggctctctttcgacccaacccatactggggggtgtgtg |
| | | ggaccgtacctggagtgcacggtatatatgcattcccgcatggcaagggcgtgctaccttgcccct |
| | | tgacgcatggtatgcgtcatcatttgccttggttaagccccatagaaacgaggcgtcacgtgccga |
| | | aaatccctttgcgtttcacagaaccatcctaaccatgggtgtagtatgggaatcgtgtatggggatga |
| | | ttaggatctctcgtagagggataggtgtgccattcaaatccagggagtactctggctctgacattgg |
| | | gacatttgatgtaaccggacctggttcagtatccgggttgtcctgtattgttacggtgtatccgtcttgg |
| | | cacactgaaagggtatttttgggtaatcctttcctactgcctgatagggtggcgtgcccggccacga |
| | | gagattaagggtagcaatttaaac |
| 45 | Swine Pasivirus 1 | gcttttgaccagtggctctgggttacttagccaagtccctttctcttattttcactagtttatgttgtgtgtt |
| | | gtctgttttgttttgtttaaattgtatacaagatccttcccgccgacacagacctcgacagtctggtgta |
| | | ggagggttggtgatattaatttgccccaaaagagtgaccgtgatacgtggaaaccatgagtacatgt |
| | | gtatccgtggaggatcgcccgggactggattccatatcccattgccatcccaacaaacggagggt |
| | | atacccaccacgtgcgcgtttgcagtgggaatctgcaaatttagtcatactgcctgatagggtgtgg |
| | | gcctgcactttggggtactcaggctgttcatataat |
| 46 | PLV-CHN | acatggggtatgttgtctgtcctgttttgttgaaacaatatataagatcctttccgccgatatagacctc |
| | | gacagtctagtgtaggaggattggtgatagtaacttgccccagaagagtgaccgtgacacataga |
| | | aaccatgagtacatgtgtatccgtggaggatcgcccgggactggattccatatcccattgccatccc |
| | | aacaaacggagggtatacccactatgtgcgcgtttgcagtgggagcctgcaaatttagtcatactg |
| | | cctgatagggtgtgggcctgcactctggggtactcaggctgtttatataat |
| 47 | Pasivirus A | tgaaaaagtggttgtgcagctggattttccggctgtgcaactgcttttgaccagtggctctgggttact |
| | (longer) | tagccaaattcctttcccttatccctattggtttgtgttgtgtgttgtttgttttgttttgtcttaactatataca |
| | | agatccttcccgccgatacagacctcgacagtctggtgtaggagggttggtgttattaatttgcccca |
| | | aaagagtgaccgtgacacgtggaaaccatgagtacatgtgtatccgtggaggatcgcccgggact |
| | | ggattccatatcccattgccatcccaacaaacggagggtatacccaccacgtgcgcgtttgcagtg |
| | | ggaatctgcaaatttagtcatactgcctgatagggtgtgggcctgcactttggggtactcaggctgtt |
| | | tatataat |
| 48 | Sicinivirus | gtgtcattaaggtgtgtttggaagttcgaattagctggtttgtggtgattagtagaccccctggaggta |
| | | cccaattcggatctgaccagggacccgtgactataccgctccggtaattcgggtttaaaacaatga |
| | | acgtcaccacacaattacttttctcattttattttcatcattgtcttcctatttaccgattacactcgatttcct |
| | | tggatgttcctggagatttccctggttacctggaccctcattattgttgttgtttcacccagcgagctgt |
| | | cccaattgcttattatttgcgcttacaacttcgtcctaatatttttctggttgatcgggttgattgagctcc |
| | | cgggctatcctgccattcaac |
| 49 | Hepacivirus K | gggaacaatggtccgtccgcggaacgactctagccatgagtctagtacgagtgcgtgccacccat |
| | | tagcacaaaaaccactgactgagccacacccctcccggaatcctgagtacaggacattcgctcgg |
| | | acgacgcatgagcctccatgccgagaaaattgggtatacccacgggtaaggggtggccacccag |
| | | cgggaatctgggggctggtcactgactatggtacagcctgatagggtgctgccgcagcgtcagtg |
| | | gtatgcggctgttcatggaac |
| 50 | Hepacivirus A | acctccgtgctaggcacggtgcgttgtcagcgttttgcgcttgcatgcgctacacgcgtcgtccaac |
| | | gcggagggaacttcacatcaccatgtgtcactccccctatggagggttccaccccgcttacacgga |
| | | aatgggttaaccatacccaaagtacgggtatgcgggtcctcctagggcccccccggcaggtcga |
| | | gggagctggaattcgtgaattcgtgagtacacgaaaatcgcggcttgaacgtctttgaccttcgga |
| | | gccgaaatttgggcgtgccccacgaaggaaggcgggggcggtgttgggccgccgccccctttat |
| | | cccacggtctgataggatgcttgcgagggcacctgccggtctcgtagaccataggac |
| 51 | BVDV1 | gtatacgagaatttgcctaggacctcgtttacaatatgggcaatctaaaattataattaggcctaagg |
| | | gacaaatcctcctcagcgaaggccgaaaagaggctagccatgcccttagtaggactagcaaaata |
| | | aggggggtagcaacagtggtgagttcgttggatggctgaagccctgagtacagggtagtcgtcag |
| | | tggttcgacgcttcggaggacaagcctcgagataccacgtggacgagggcatgcccacagcaca |
| | | tcttaacctggacgggggtcgttcaggtgaaaacggtttaaccaaccgctacgaatacagcctgat |
| | | agggtgctgcagaggcccactgtattgctactgaaaatctctgctgtacatggcac |
| 52 | Border Disease | gtatacgggagtagctcatgcccgtatacaaaattggatattccaaaactcgattgggttagggagc |
| | Virus | cctcctagcgacggccgaaccgtgttaaccatacacgtagtaggactagcagacgggaggacta |
| | | gccatcgtggtgagatccctgagcagtctaaatcctgagtacaggatagtcgtcagtagttcaacg |
| | | caggcacggttctgccttgagatgctacgtggacgagggcatgcccaagacttgctttaatctcgg |
| | | cgggggtcgccgaggtgaaaacacctaacggtgttggggttacagcctgatagggtgctgcaga |
| | | ggcccacgaataggctagtataaaaatctctgctgtacatggcac |
| 53 | BVDV2 | gtatacgagattagctaaagtactcgtatatggattggacgtcaacaaatttttaattggcaacgtagg |
| | | gaaccttcccctcagcgaaggccgaaaagaggctagccatgccctttagtaggactagcaaaagt |
| | | agggggactagcggtagcagtgagttcgttggatggccgaacccctgagtacaggggagtcgtc |
| | | aatggttcgacactccattagtcgaggagtctcgagatgccatgtggacgagggcatgcccacgg |
| | | cacatcttaacccatgcgggggttgcatgggtgaaagcgctaatcgtggcgttatggacacagcct |
| | | gatagggtgtagcagagacctgctattccgctagtaaaaaactctgctgtacatggcac |
| 54 | CSFV-PK15C | gtatacgaggttagttcattctcgtatgcattattggacaaatcaaaatttcaatttggttcagggcctc |
| | | cctccagcgacggccgaactgggctagccatgcccatagtaggactagcaaacggagggacta |
| | | gccgtagtggcgagctccctgggtgttctaagtcctgagtacaggacagtcgtcagtagttcgacg |
| | | tgagcagaagcccacctcgagatgctatgtggacgagggcatgcccaagacgcaccttaaccct |
| | | agcgggggtcgctagggtgaaatcacaccacgtgatgggagtccgacctgatagggtgctgcag |
| | | aggctcactattaggctagtataaaaatctctgctgtacatggcac |
| 55 | SF573 | aaaaccgaccccagagatcagaaagtcgttgacgcgatcttttattagaggacgttgcgctggcgc |
| | Dicistrovirus | gagctttaattagcagacgccaaaaataaacaacaaaatgctgatcgcgagacttaattgtcagac |
| | | gattggccaaatccgatgtgatctttgctgctcccagattgccgaaataggagtagtag |
| 56 | Hubei Picorna- | ccccaaaaccccccccttaaactcaacactgtagtggattcattttccgttgcaaaacaaaacattac |
| | like Virus | tacccgcatttatgtaggctctgtgttttctatgcgaccgttacattaatctctactctgacccactagttt |
| | | ataaaaccgaagacctgaatgaaacgattttccttcttttcaacctctaacgaacctctgacggcttga |
| | | gaaacctgaagttagtaattatgtttaaaagaaaggaaagtcaaacgcgatgactcttacatccctat |
| | | tccataccgttgctccacaatgtgagcgatgcgaggtcgggactgcagtattaggggaacgagct |
| | | acatggagagttaattatctctcccctcctacgggagtctcatgtgagctgtagaaagcggttggca |
| | | cctctcgttacctcgcctgtacatgatcc |
| 57 | CRPV | aaaagcaaaaatgtgatcttgcttgtaaatacaattttgagaggttaataaattacaagtagtgctatttt |
| | | tgtatttaggttagctatttagctttacgttccaggatgcctagtggcagccccacaatatccaggaag |
| | | ccctctctgcggtttttcagattaggtagtcgaaaaacctaagaaatttacct |
| 58 | Salivirus A BN5 | tttcctcctttcgaccgccttacggcaggcgggtccgcggacggcttcggcctacccgcgacaag |
| | | aatgccgtcatctgtccttatcacccatattctttcccttcccccgcaaccatcacgcttactcgcgca |
| | | cgtgttgagtggcacgtgcgttgtccaaacagttacactcacacccttggggcgggtttgtcccgcc |
| | | ctcgggttcctcgcggaaccctccctcttctctctccctttctatccgccttcactttccataactacagt |
| | | gctttggtaggtaagcatcctgaccccccgcggaagctgccaacgtggcaactgtggggatccag |
| | | gcaggttatcaaaggcacccggtctttccgccttcaggagtatccctgccggtgaattccgacagg |
| | | gctctgcttggtgccaacctcccccaaatgcgcgctgcgggagtgctcttccccaactcatcttagt |
| | | aacctctcatgtgtgtgcttggtcagcatatctgaggcgacgttccgctgtcccagaccagtccagc |
| | | aatggacgggccagtgtgcgtagtcgctttccggtttcccggcgcatgtttggcgaaacgctgagg |
| | | taaggttggtgtgcccaatgcccgtaatttggtgacacctcaagaccacccaggaatgccaggga |
| | | ggtaccccacttcggtgggatctgaccctgggctaattgtctacggtggttcttcttgcttccacttctc |
| | | ttttttctggcatg |
| 59 | Salivirus A BN2 | tatggcaggcgggcttgtggacggcttcggcccacccacagcaagaatgccatcatctgtcctca |
| | | cccccatgtttcccctttctttccctgcaaccgttacgcttactcgcaggtgcatttgagtggtgcacgt |
| | | gttgaataaacagctacactcacatgggggcgggttttcccgccctgcggcctctcgcgaggccc |
| | | acccctccccttcctcccataactacagtgctttggtaggtaagcatcctgatcccccgcggaagct |
| | | gctcacgtggcaactgtggggacccagacaggttatcaaaggcacccggtctttccgccttcagg |
| | | agtatccctgctagtgaattctagtagggctctgcttggtgccaacctcccccaaatgcgcgctgcg |
| | | ggagtgctcttccccaactcaccctagtatcctctcatgtgtgtgcttggtcagcatatctgagacgat |
| | | gttccgctgtcccagaccagtccagtaatggacgggccagtgtgcgtagtcgtcttccggcttttcc |
| | | ggcgcatgtttggtgaaccggtggggtaaggttggtgtgcccaacgcccgtactttggtgatacct |
| | | caagaccacccaggaatgccagggaggtaccccgcttcacagcgggatctgaccctgggctaat |
| | | tgtctacggtggttcttcttgcttccacttctttctactgttc |
| 60 | Salivirus A | tttcgaccgccttatggcaggcgggcttgtggacggcttcggcccacccacagcaagaatgccat |
| | 02394 | catctgtcctcacccccatttctcccctccttcccctgcaaccattacgcttactcgcatgtgcattgag |
| | | tggtgcacgtgttgaacaaacagctacactcacgtgggggcgggttttcccgcccttcggcctctc |
| | | gcgaggcccacccttccccttcctcccataactacagtgctttggtaggtaagcatcctgatccccc |
| | | gcggaagctgctcgcgtggcaactgtggggacccagacaggttatcaaaggcacccggtctttcc |
| | | gcctccaggagtatccctgctagtgaattctagtggggctctgcttggtgccaacctcccccaaatg |
| | | cgcgctgcgggagtgctcttccccaactcaccctagtatcctctcatgtgtgtgcttggtcagcatat |
| | | ctgagacgatgttccgctgtcccagaccagtccagcaatggacgggccagtgtgcgtagtcgtctt |
| | | ccggcttgtccggcgcatgtttggtgaaccggtggggtaaggttggtgtgcccaacgcccgtactt |
| | | tggtgacaactcaagaccacccaggaatgccagggaggtaccccgcctcacggcgggatctga |
| | | ccctgggctaattgtctacggtggttcttcttgcttccatttctttcttctgttc |
| 61 | Salivirus A GUT | tatggcaggcgggcttgtggacggtttcggcccacccacagcaagaatgccatcatctgtcctcac |
| | | ccccaattttccctttcttcccctgcaatcatcacgcttactcgcatgtgcattgagtggtgcatgtgtt |
| | | gaacaaacagctacactcacatgggggcgggttttcccgccctacggcctctcgcgaggcccac |
| | | ccttcccctccccttataactacagtgctttggcaggtaagcatcctgatcccccgcggaagctgct |
| | | cacgtggcaactgtggggacccagacaggttatcaaaggcacccggtctttccgccttcaggagc |
| | | atccccactagtgaattctagtggggctctgcttggtgccaacctcccccaaatgcgcgctgcggg |
| | | agtgctcttccccaacccatcctagtatcctctcatgtgtgtgcttggtcagcatatctgagacgacgt |
| | | tccgctgtcccagaccagtccagtaatggacgggccagtgtgcgtagtcgtcttccggcttgtccg |
| | | gcgcatgtttggtgaaccggtggggtaaggttggtgtgcccaacgcccgtactttggtgacacctc |
| | | aagaccacccaggaatgccagggaggtaccccgcctcacggcgggatctgaccctgggctaatt |
| | | gtctacggtggttcttcttgcttccacttctttctt |
| 62 | Salivirus A CH | ttctcctgcaaccattacgcttaatcgcatgtgcattgagtggtgcatgtgttgaacaaacagctaca |
| | | atcacatgggggcgggttttcccgccccacggcttctcgcgaggcccatccctcccttttctcccat |
| | | aactacagtgctttggtaggtaagcatcccgatctcccgcggaagctgctcacgtggcaactgtgg |
| | | ggacccagacaggttatcaaaggcacccggtctttccgccttcaggagtatccctgctagcgaatt |
| | | ctagtagggctctgcttggtgccaacctctcccaaatgcgcgctgcgggagtgctcttccccaaatc |
| | | accccagtatcctctcatgtgtgtgcctggtcagcatatctgagacgatgttccgctgtcccagacca |
| | | gtccagtaatggacgggccagtgtgcgtagtcgtcctccggcttgtccggcgcatgtttggtgaac |
| | | cggtggggtaaggttggtgtgcccaacgcccgtaatcaggggatacctcaaggcacccaggaat |
| | | gccagggaggtatcccgcctcacagcgggatctgaccctggggtaaatgtctgcggggggtcct |
| | | cttggcccaattctcagtaattttcagg |
| 63 | Salivirus A SZ1 | tctgtcctcaccccatcttcccttctttcctgcaccgttacgcttactcgcatgtgcattgagtggtgca |
| | | cgtgcttgaacaaacagctacactcacatgggggcgggttttcccgccctgcggcctctcgcgag |
| | | gcccacccctccccttcctcccataactacagtgctttggtaggtaagcatcctgatcccccgcgga |
| | | agctgctcacgtggcaactgtggggacccagacaggttatcaaaggcacccggtctttccgccttc |
| | | aggagtatccctgctagtgaattctagtagggctctgcttggtgccaacctcccccaaatgcgcgct |
| | | gcgggagtgctcttccccaactcaccctagtatcctctcatgtgtgtgcttggtcagcatatctgaga |
| | | cgatgttccgctgtcccagaccagtccagtaatggacgggccagtgtgcgtagtcgtcttccggctt |
| | | gtccggcgcatgtttggtgaaccggtggggtaaggttggtgtgcccaacgcccgtactttggtgat |
| | | acctcaagaccacccaggaatgccagggaggtaccccgcttcacagcgggatctgaccctggg |
| | | ctaattgtctacggtggttcttcttgcttccacttctttctactgttcatg |
| 64 | Salivirus FHB | acatggggggtctgcggacggcttcggcccacccgcgacaagaatgccgtcatctgtcctcatta |
| | | cccgtattccttcccttcccccgcaaccaccacgcttactcgcgcacgtgttgagtggcacgtgcgt |
| | | tgtccaaacagctacacccacacccttcggggcgggtttgtcccgccctcgggttcctcgcggaa |
| | | cccccccctccctctctctctttctatccgccctcacttcccataactacagtgctttggtaggtgagc |
| | | accctgaccccccgcggaagctgctaacgtggcaactgtggggatccaggcaggttatcaaagg |
| | | cacccggtctttccgccttcaggagtatctctgccggtgaattccggtagggctctgcttggtgcca |
| | | acctcccccaaatgcgcgctgcgggagtgctcttccccaactcatcttagtaacctctcatgtgtgtg |
| | | cttggtcagcatatctgaggcgacgttccgctgtcccagaccagtccagcaatggacgggccagt |
| | | gtgcgtagtcgctttccggttttccggcgcatgtttggcgaaacgctgaggtaaggttggtgtgccc |
| | | aacgcccgtaatttggtgatacctcaagaccacccaggaatgccagggaggtaccccacttcggt |
| | | gggatctgaccctgggctaattgtctacggtggttcttcttgcttccacttctcttttttctggcatg |
| 65 | CVB3 | ttaaaacagcctgtgggttgatcccacccacaggcccattgggcgctagcactctggtatcacggt |
| | | acctttgtgcgcctgttttataccccctcccccaactgtaacttagaagtaacacacaccgatcaaca |
| | | gtcagcgtggcacaccagccacgttttgatcaagcacttctgttaccccggactgagtatcaataga |
| | | ctgctcacgcggttgaaggagaaagcgttcgttatccggccaactacttcgaaaaacctagtaaca |
| | | ccgtggaagttgcagagtgtttcgctcagcactaccccagtgtagatcaggtcgatgagtcaccgc |
| | | attccccacgggcgaccgtggcggtggctgcgttggcggcctgcccatggggaaacccatggg |
| | | acgctctaatacagacatggtgcgaagagtctattgagctagttggtagtcctccggcccctgaatg |
| | | cggctaatcctaactgcggagcacacaccctcaagccagagggcagtgtgtcgtaacgggcaac |
| | | tctgcagcggaaccgactactttgggtgtccgtgtttcattttattcctatactggctgcttatggtgac |
| | | aattgagagatcgttaccatatagctattggattggccatccggtgactaatagagctattatatatcc |
| | | ctttgttgggtttataccacttagcttgaaagaggttaaaacattacaattcattgttaagttgaatacag |
| | | caaa |
| 66 | CVB1 | ttaaaacagcctgtgggttgttcccacccacaggcccattgggcgctagcactctggtatcacggta |
| | | cctttgtgcgcctgttttacatcccctccccaaattgtaatttagaagtttcacacaccgatcattagca |
| | | agcgtggcacaccagccatgttttgatcaagcacttctgttaccccggactgagtatcaatagaccg |
| | | ctaacgcggttgaaggagaaaacgttcgttacccggccaactacttcgaaaaacctagtaacacca |
| | | tggaagttgcggagtgtttcgctcagcactaccccagtgtagatcaggtcgatgagtcaccgcgttc |
| | | cccacgggcgaccgtggcggtggctgcgttggcggcctgcctacggggaaacccgtaggacg |
| | | ctctaatacagacatggtgcgaagagtctattgagctagttggtaatcctccggcccctgaatgcgg |
| | | ctaatcctaactgcggagcacataccctcaaaccagggggcagtgtgtcgtaacgggcaactctg |
| | | cagcggaaccgactactttgggtgtccgtgtttcattttattcctatactggctgcttatggtgacaatt |
| | | gacaggttgttaccatatagttattggattggccatccggtgactaacagagcaattatatatctctttg |
| | | ttgggtttataccacttagcttgaaagaggttaaaacactacatctcatcattaaactaaatacaacaa |
| | | a |
| 67 | Echovirus 7 | ttaaaacagcctgtgggttgttcccacccacagggcccattgggcgtcagcaccctggtatcacgg |
| | | tacctttgtgcgcctgttttatatcccttcccccaattgtaacttagaagaaacacacaccgatcaaca |
| | | gcaagcgtggcacaccagccatgttttggtcaagcacttctgttaccccggactgagtatcaataga |
| | | ctgctcacgcggttgaaggagaaagcgtccgttatccggccagctacttcgagaaacctagtaac |
| | | accatggaagttgcggagtgtttcgctcagcactaccccagtgtagatcaggtcgatgagtcaccg |
| | | ctttccccacgggcgaccgtggcggtggctgcgttggcggcctgcctatgggggaacccatagg |
| | | acgctctaatacagacatggtgcgaagagtctattgagctagctggtattcctccggcccctgaatg |
| | | cggctaatcctaactgtggagcacatgcccctaatccaaggggtagtgtgtcgtaatgagcaattcc |
| | | gcagcggaaccgactactttgggtgtccgtgtttcctcttattcttgtactggctgcttatggtgacaat |
| | | tgagagattgttaccatatagctattggattggccatccggtgactaatagagctattgtgtatctcttt |
| | | gttggatttgtaccacttaatttgaaagaaatcaggacactacgctacattttactattgaacaccgca |
| | | aa |
| 68 | CVB5 | ttaaaacagcctgtgggttgtacccacccacagggcccactgggcgctagcactctggtatcacg |
| | | gtacctttgtgcgcctgttttatgcccccttcccccaattgaaacttagaagttacacacaccgatcaa |
| | | cagcgggcgtggcataccagccgcgtcttgatcaagcactcctgtttccccggaccgagtatcaat |
| | | agactgctcacgcggttgaaggagaaaacgttcgttacccggctaactacttcgagaaacctagta |
| | | gcatcatgaaagttgcgaagcgtttcgctcagcacatccccagtgtagatcaggtcgatgagtcac |
| | | cgcattccccacgggcgaccgtggcggtggctgcgttggcggcctgcctacggggcaacccgt |
| | | aggacgcttcaatacagacatggtgcgaagagtcgattgagctagttagtagtcctccggcccctg |
| | | aatccggctaatcctaactgcggagcacataccctcaacccagggggcattgtgtcgtaacgggt |
| | | aactctgcagcggaaccgactactttgggtgtccgtgtttccttttattcttataatggctgcttatggtg |
| | | acaattgaaagattgttaccatatagctattggattggccatccggtgtctaacagagctattatatac |
| | | ctctttgttggatttgtaccacttgatctaaaggaagtcaagacactacaattcatcatacaattgaaca |
| | | cagcaaa |
| 69 | EVA71 | ttaaaacagcctgtgggttgcacccactcacagggcccactgggcgcaagcactctggcacttcg |
| | | gtacctttgtgcgcctgttttatatcccctcccccaatgaaatttagaagcagcaaaccccgatcaata |
| | | gcaggcataacgctccagttatgtcttgatcaagcacttctgtttccccggactgagtatcaatagac |
| | | tgctcacgcggttgaaggagaaaacgttcgttatccggctaactacttcggaaagcctagtaacac |
| | | catggaagttgcggagagtttcgttcagcacttccccagtgtagatcaggtcgatgagtcaccgcat |
| | | tccccacgggcgaccgtggcggtggctgcgttggcggcctgcccatggggtaacccatgggac |
| | | gctctaatacggacatggtgtgaagagtctactgagctagttagtagtcctccggcccctgaatgcg |
| | | gctaatcccaactgcggagcacacgcccacaagccagtgggtagtgtgtcgtaacgggcaactct |
| | | gcagcggaaccgactactttgggtgtccgtgtttccttttattcttatgttggctgcttatggtgacaatt |
| | | aaagagttgttaccatatagctattggattggccatccggtgtgcaacagagcgatcgtttacctattt |
| | | attggttttgtaccattgacactgaagtctgtgatcacccttaattttatcttaaccctcaacacagccaa |
| | | ac |
| 70 | CVA3 | ttaaaacagcctgtgggttgtacccacccacagggcccactgggcgctagcacactggtattacg |
| | | gtacctttgtgcgcctgttttataccccccccaacctcgaaacttagaagtaaagcaaacccgatca |
| | | atagcaggtgcggcgcaccagtcgcatcttgatcaagcacttctgtaaccccggaccgagtatcaa |
| | | tagactgctcacgcggttgaaggagaaaacgttcgttacccggctaactacttcgagaaacccagt |
| | | agcatcatgaaagttgcagagtgtttcgctcagcactacccccgtgtagatcaggccgatgagtca |
| | | ccgcacttccccacgggcgaccgtggcggtggctgcgttggcggcctgcctatggggcaaccca |
| | | taggacgctctaatacggacatggtgcgaagagtctattgagctagttagtagtcctccggcccctg |
| | | aatgcggctaatcctaactgcggagcacatacccttaatccaaagggcagtgtgtcgtaacgggta |
| | | actctgcagcggaaccgactactttgggtgtccgtgtttccttttaatttttactggctgcttatggtgac |
| | | aattgaggaattgttgccatatagctattggattggccatccggtgactaacagagctattgtgttcca |
| | | atttgttggatttaccccgctcacactcacagtcgtaagaacccttcattacgtgttatttctcaactcaa |
| | | gaaa |
| 71 | CVA12 | ttaaaacagcctgtgggttgtacccacccacagggcccactgggcgctagcactctggtactacg |
| | | gtacctttgtgtgcctgttttaagcccctaccccccactcgtaacttagaaggcttctcacactcgatc |
| | | aatagtaggtgtggcacgccagtcacaccgtgatcaagcacttctgttaccccggtctgagtacca |
| | | ataagctgctaacgcggctgaaggggaaaacgatcgttatccggctaactacttcgagaaaccca |
| | | gtaccaccatgaacgttgcagggtgtttcgctcggcacaaccccagtgtagatcaggtcgatgagt |
| | | caccgtattccccacgggcgaccgtggcggtggctgcgttggcggcctgcccatggggtgaccc |
| | | atgggacgctctaatactgacatggtgcgaagagtctattgagctagttagtagtcctccggcccct |
| | | gaatgcggctaatcctaactgcggagcacatacccttaatccaaagggcagtgtgtcgtaacggg |
| | | caactctgcagcggaaccgactactttgggtgtccgtgtttccttttattcttacattggctgcttatggt |
| | | gacaattgaaaagttgttaccatatagctattggattggccatccggtgacaaatagagctattgtata |
| | | tctttttgttggttacgtaccccttaattacaaagtggtttcaactttgaaatacatcctaacactaaattg |
| | | tagaaa |
| 72 | EV24 | ttaaaacagcctgtgggttgcacccacccacagggcccacagggcgctagcactctggtatcacg |
| | | gtacctttgtgcgcctgttttattaccccttccccaattgaaaattagaagcaatgcacaccgatcaac |
| | | agcaggcgtggcgcaccagtcacgtctcgatcaagcacttctgtttccccggaccgagtatcaata |
| | | gactgctcacgcggttgaaggagaaagtgttcgttatccggctaaccacttcgagaaacccagtaa |
| | | caccatgaaagttgcagggtgtttcgctcagcacttccccagtgtagatcaggtcgatgagtcacc |
| | | gcgttccccacgggcgaccgtggcggtggctgcgttggcggcctgcctatgggttaacccatag |
| | | gacgctctaatacagacatggtgcgaagagtttattgagctggttagtatccctccggcccctgaat |
| | | gcggctaatcctaactgcggagcacgtgcctccaatccagggggttgcatgtcgtaacgggtaac |
| | | tctgcagcggaaccgactactttgggtgtccgtgtttccttttattcttatactggctgcttatggtgaca |
| | | atcgaggaattgttaccatatagctattggattggccatccggtgtctaacagagcgattatatacctc |
| | | tttgttggatttatgcagctcaataccaccaactttaacacattgaaatatatcttaaagttaaacacag |
| | | caaa |
| 348 | AP1.0 | attctcgggctacggccctggagccactccggctcctaaagatttagaagtttgagcacacccgcc |
| | | cactagggccccccatccaggggggcaacgggcaagcacttctgtttccccggtatgatctgata |
| | | ggctgtaaccacggctgaaacagagattatcgttatccgcttcactacttcgagaagcctagtaatg |
| | | atgggtgaaattgaatccgttgatccggtgtctcccccacaccagaaactcatgatgagggttgcca |
| | | tcccggctacggcgacgtagcgggcatccctgcgctggcatgaggcctcttaggaggacggatg |
| | | atatggatcttgtcgtgaagagcctattgagctagtgtcgactcctccgcccccgtgaatgcggcta |
| | | atcctaaccccggagcaggtgggtccaatccagggcctggcctgtcgtaatgcgtaagtctggga |
| | | cggaaccgactactttcgggaaggcgtgtttccatttgttcattatttgtgtgtttatggtgacaactctg |
| | | ggtaaacgttctattgcgtttattgagagattcccaacaattgaacaaacgagaactacctgttttatta |
| | | aatttacacagagaagaattaca |
| 349 | CK1.0 | gtggccacgcccgggccaccgatacttcccttcactccttcgggactgttggggaggaacacaac |
| | | agggctcccctgttttcccattccttcccccttttcccaaccccaaccgccgtatctggtggcggcaa |
| | | gacacacgggtctttccctctaaagcacaattgtgtgtgtgtcccaggtcctcctgcgtacggtgcg |
| | | ggagtgctcccacccaactgttgtaagcctgtccaacgcgtcgtcctggcaagactatgacgtcgc |
| | | atgttccgctgcggatgccgaccgggtaaccggttccccagtgtgtgtagtgcgatcttccaggtc |
| | | ctcctggttggcgttgtccagaaactgcttcaggtaagtggggtgtgcccaatccctacaaaggttg |
| | | attctttcaccaccttaggaatgctccggaggtaccccagcaacagctgggatctgaccggaggct |
| | | aattgtctacgggtggtgtttcctttttcttttcacacaactctactgctgacaactcactgactatccact |
| | | tgctctgTcacG |
| 350 | PV1.0 | aacaaaaggctacaccacttgggctacggcccgcgccaccttgtggcgcaaagacattagaaga |
| | | atagcataccgcccactagggccctgcagccagcagggtaacgggcaagcacttctgtctcccc |
| | | ggtagaacggtataggctgtacccacggccgaaaactgaactatcgttacccgactccgtacttcg |
| | | caaagcttagtaggaaactggaaagttcgagttattgacccggagtgttccccccactccagaaac |
| | | gcgtgatgagggttgccaccccgaccatggcgacatggtgggcatccctgcgctggcacgcgg |
| | | cctctaagaggataactcgctcctactggtaaccgaagagccccgtgagctacggtttattcctccg |
| | | cctccctgaatgcggctaatcctaacccatgagcagttgccatagatccatatggtggactgtcgta |
| | | acgcgtaagttgtgggcggaaccgactactttgggatggcgtgtttccttgttttctccatttgttgttgt |
| | | atggtgacaagttatagatctcgatctatagcgtttcttgagagatttccaaacatttattcaagtcgta |
| | | caattcttgtgtttaagcagtacagtgtaacc |
| 351 | SV1.0 | tctgtcctcaccccatcttcccttctttcctgcaccgttacgcttactcgcatgtgcattgagtggtgca |
| | | cgtgcttgaacaaacagctacactcacatgggggcgggttttcccgccctgcggcctctcgcgag |
| | | gcccacccctccccttcctcccataactacagtgctttggtaggtaagcatcctgatcccccgcgga |
| | | agctgctcacgtggcaactgtggggacccagacaggttatcaaaggcacccggtctttccgccttc |
| | | aggagtatccctgctagtgaattctagtagggctctgcttggtgccaacctcccccaaatgcgcgct |
| | | gcgggagtgctcttccccaactcaccctagtatcctctcatgtgtgtgcttggtcagcatatctgaga |
| | | cgatgttccgctgtcccagaccagtccagtaatggacgggccagtgtgcgtagtcgtcttccggctt |
| | | gtccggcgcatgtttggtgaaccggtggggtaaggttggtgtgcccaacgcccgtactttggtgat |
| | | acctcaagaccacccaggaatgccagggaggtaccccgcttcacagcgggatctgaccctggg |
| | | ctaattgtctacggtggttcttcttgcttccacttctttctactgttcgccacc |
| 352 | Caprine | gtggccacgcccgggccaccgatacttcccttcactccttcgggactgttggggaggaacacaac |
| | Kobuvirus 5Δ40 | agggctcccctgttttcccattccttcccccttttcccaaccccaaccgccgtatctggtggcggcaa |
| | | gacacacgggtctttccctctaaagcacaattgtgtgtgtgtcccaggtcctcctgcgtacggtgcg |
| | | ggagtgctcccacccaactgttgtaagcctgtccaacgcgtcgtcctggcaagactatgacgtcgc |
| | | atgttccgctgcggatgccgaccgggtaaccggttccccagtgtgtgtagtgcgatcttccaggtc |
| | | ctcctggttggcgttgtccagaaactgcttcaggtaagtggggtgtgcccaatccctacaaaggttg |
| | | attctttcaccaccttaggaatgctccggaggtaccccagcaacagctgggatctgaccggaggct |
| | | aattgtctacgggtggtgtttcctttttcttttcacacaactctactgctgacaactcactgactatccact |
| | | tgctctcttgtgcctttctgctctggttcaagttccttgattgtttttgactgcttttcactgcttttcttctca |
| | | caatccttgctcagttcaaagtc |
| 353 | Caprine | gtggccacgcccgggccaccgatacttcccttcactccttcgggactgttggggaggaacacaac |
| | Kobuvirus | agggctcccctgttttcccattccttcccccttttcccaaccccaaccgccgtatctggtggcggcaa |
| | 5Δ40/3Δ122 | gacacacgggtctttccctctaaagcacaattgtgtgtgtgtcccaggtcctcctgcgtacggtgcg |
| | | ggagtgctcccacccaactgttgtaagcctgtccaacgcgtcgtcctggcaagactatgacgtcgc |
| | | atgttccgctgcggatgccgaccgggtaaccggttccccagtgtgtgtagtgcgatcttccaggtc |
| | | ctcctggttggcgttgtccagaaactgcttcaggtaagtggggtgtgcccaatccctacaaaggttg |
| | | attctttcaccaccttaggaatgctccggaggtaccccagcaacagctgggatctgaccggaggct |
| | | aattgtctacgggtggtgtttcctttttcttttcacacaactaaagtc |
| 354 | Caprine | gtggccacgcccgggccaccgatacttcccttcactccttcgggactgttggggaggaacacaac |
| | Kobuvirus | agggctcccctgttttcccattccttcccccttttcccaaccccaaccgccgtatctggtggcggcaa |
| | 5Δ40/3Δ86_Dista | gacacacgggtctttccctctaaagcacaattgtgtgtgtgtcccaggtcctcctgcgtacggtgcg |
| | 1 | ggagtgctcccacccaactgttgtaagcctgtccaacgcgtcgtcctggcaagactatgacgtcgc |
| | | atgttccgctgcggatgccgaccgggtaaccggttccccagtgtgtgtagtgcgatcttccaggtc |
| | | ctcctggttggcgttgtccagaaactgcttcaggtaagtggggtgtgcccaatccctacaaaggttg |
| | | attctttcaccaccttaggaatgctccggaggtaccccagcaacagctgggatctgaccggaggct |
| | | aattgtctacgggtggtgtttcctttttcttttcacacaactctactgctgacaactcactgactatccact |
| | | tgctctaaagtc |
| 355 | Caprine | gtggccacgcccgggccaccgatacttcccttcactccttcgggactgttggggaggaacacaac |
| | Kobuvirus | agggctcccctgttttcccattccttcccccttttcccaaccccaaccgccgtatctggtggcggcaa |
| | 5Δ40/3Δ | gacacacgggtctttccctctaaagcacaattgtgtgtgtgtcccaggtcctcctgcgtacggtgcg |
| | 122_Kozak | ggagtgctcccacccaactgttgtaagcctgtccaacgcgtcgtcctggcaagactatgacgtcgc |
| | | atgttccgctgcggatgccgaccgggtaaccggttccccagtgtgtgtagtgcgatcttccaggtc |
| | | ctcctggttggcgttgtccagaaactgcttcaggtaagtggggtgtgcccaatccctacaaaggttg |
| | | attctttcaccaccttaggaatgctccggaggtaccccagcaacagctgggatctgaccggaggct |
| | | aattgtctacgggtggtgtttcctttttcttttcacacaactgccacc |
| 356 | Caprine | gtggccacgcccgggccaccgatacttcccttcactccttcgggactgttggggaggaacacaac |
| | Kobuvirus | agggctcccctgttttcccattccttcccccttttcccaaccccaaccgccgtatctggtggcggcaa |
| | 5Δ40/3Δ86 | gacacacgggtctttccctctaaagcacaattgtgtgtgtgtcccaggtcctcctgcgtacggtgcg |
| | Proximal | ggagtgctcccacccaactgttgtaagcctgtccaacgcgtcgtcctggcaagactatgacgtcgc |
| | | atgttccgctgcggatgccgaccgggtaaccggttccccagtgtgtgtagtgcgatcttccaggtc |
| | | ctcctggttggcgttgtccagaaactgcttcaggtaagtggggtgtgcccaatccctacaaaggttg |
| | | attctttcaccaccttaggaatgctccggaggtaccccagcaacagctgggatctgaccggaggct |
| | | aattgtctacgggtggtgtttcctttttcttttcacacaactttcactgcttttcttctcacaatccttgctca |
| | | gttcaaagtc |
| 357 | Parabovirus | tgaaccgttacgcaccactcagttggtgtttggtggcaccaatgatggaacaaaaggctacaccac |
| | | ttgggctacggcccgcgccaccttgtggcgcaaagacattagaagaatagcataccgcccactag |
| | | ggccctgcagccagcagggtaacgggcaagcacttctgtctccccggtagaacggtataggctgt |
| | | acccacggccgaaaactgaactatcgttacccgactccgtacttcgcaaagcttagtaggaaactg |
| | | gaaagttcgagttattgacccggagtgttccccccactccagaaacgcgtgatgagggttgccacc |
| | | ccgaccatggcgacatggtgggcatccctgcgctggcacgcggcctctaagaggataactcgct |
| | | cctactggtaaccgaagagccccgtgagctacggtttattcctccgcctccctgaatgcggctaatc |
| | | ctaacccatgagcagttgccatagatccatatggtggactgtcgtaacgcgtaagttgtgggcgga |
| | | accgactactttgggatggcgtgtttccttgttttctccatttgttgttgtatggtgacaagttatagatct |
| | | cgatctatagcgtttcttgagagatttccaaacatttattcaagtcgtacaattcttgtgtttaagcagta |
| | | cagtgtaagg |
| 358 | Parabovirus 5Δ48 | aacaaaaggctacaccacttgggctacggcccgcgccaccttgtggcgcaaagacattagaaga |
| | | atagcataccgcccactagggccctgcagccagcagggtaacgggcaagcacttctgtctcccc |
| | | ggtagaacggtataggctgtacccacggccgaaaactgaactatcgttacccgactccgtacttcg |
| | | caaagcttagtaggaaactggaaagttcgagttattgacccggagtgttccccccactccagaaac |
| | | gcgtgatgagggttgccaccccgaccatggcgacatggtgggcatccctgcgctggcacgcgg |
| | | cctctaagaggataactcgctcctactggtaaccgaagagccccgtgagctacggtttattcctccg |
| | | cctccctgaatgcggctaatcctaacccatgagcagttgccatagatccatatggtggactgtcgta |
| | | acgcgtaagttgtgggcggaaccgactactttgggatggcgtgtttccttgttttctccatttgttgttgt |
| | | atggtgacaagttatagatctcgatctatagcgtttcttgagagatttccaaacatttattcaagtcgta |
| | | caattcttgtgtttaagcagtacagtgtaagg |
| 359 | Parabovirus 5Δ67 | tgggctacggcccgcgccaccttgtggcgcaaagacattagaagaatagcataccgcccactag |
| | | ggccctgcagccagcagggtaacgggcaagcacttctgtctccccggtagaacggtataggctgt |
| | | acccacggccgaaaactgaactatcgttacccgactccgtacttcgcaaagcttagtaggaaactg |
| | | gaaagttcgagttattgacccggagtgttccccccactccagaaacgcgtgatgagggttgccacc |
| | | ccgaccatggcgacatggtgggcatccctgcgctggcacgcggcctctaagaggataactcgct |
| | | cctactggtaaccgaagagccccgtgagctacggtttattcctccgcctccctgaatgcggctaatc |
| | | ctaacccatgagcagttgccatagatccatatggtggactgtcgtaacgcgtaagttgtgggcgga |
| | | accgactactttgggatggcgtgtttccttgttttctccatttgttgttgtatggtgacaagttatagatct |
| | | cgatctatagcgtttcttgagagatttccaaacatttattcaagtcgtacaattcttgtgtttaagcagta |
| | | cagtgtaagg |
| 360 | Parabovirus 3Δ60 | tgaaccgttacgcaccactcagttggtgtttggtggcaccaatgatggaacaaaaggctacaccac |
| | | ttgggctacggcccgcgccaccttgtggcgcaaagacattagaagaatagcataccgcccactag |
| | | ggccctgcagccagcagggtaacgggcaagcacttctgtctccccggtagaacggtataggctgt |
| | | acccacggccgaaaactgaactatcgttacccgactccgtacttcgcaaagcttagtaggaaactg |
| | | gaaagttcgagttattgacccggagtgttccccccactccagaaacgcgtgatgagggttgccacc |
| | | ccgaccatggcgacatggtgggcatccctgcgctggcacgcggcctctaagaggataactcgct |
| | | cctactggtaaccgaagagccccgtgagctacggtttattcctccgcctccctgaatgcggctaatc |
| | | ctaacccatgagcagttgccatagatccatatggtggactgtcgtaacgcgtaagttgtgggcgga |
| | | accgactactttgggatggcgtgtttccttgttttctccatttgttgttgtatggtgacaagttatagatct |
| | | cgatctatagcgtttgtaagg |
| 361 | Apodemus | tttgaaaggggtgcggatatcatggcgtttctcgccatgatatccgcacattgcaaacccatattgca |
| | Picornavirus | tacccactgggtatgcattatggggaggcccctttcacccctccccccccaattaccttttccccctct |
| | | agtaaccatacgctttactcagcgtaactactccgggttacgtgatgaagaagaggctacggagatt |
| | | ctcgggctacggccctggagccactccggctcctaaagatttagaagtttgagcacacccgccca |
| | | ctagggccccccatccaggggggcaacgggcaagcacttctgtttccccggtatgatctgatagg |
| | | ctgtaaccacggctgaaacagagattatcgttatccgcttcactacttcgagaagcctagtaatgatg |
| | | ggtgaaattgaatccgttgatccggtgtctcccccacaccagaaactcatgatgagggttgccatcc |
| | | cggctacggcgacgtagcgggcatccctgcgctggcatgaggcctcttaggaggacggatgata |
| | | tggatcttgtcgtgaagagcctattgagctagtgtcgactcctccgcccccgtgaatgcggctaatc |
| | | ctaaccccggagcaggtgggtccaatccagggcctggcctgtcgtaatgcgtaagtctgggacg |
| | | gaaccgactactttcgggaaggcgtgtttccatttgttcattatttgtgtgtttatggtgacaactctgg |
| | | gtaaacgttctattgcgtttattgagagattcccaacaattgaacaaacgagaactacctgttttattaa |
| | | atttacacagagaagaattaca |
| 362 | Apodemus | cccctccccccccaattaccttttccccctctagtaaccatacgctttactcagcgtaactactccggg |
| | Picornavirus | ttacgtgatgaagaagaggctacggagattctcgggctacggccctggagccactccggctccta |
| | 5Δ105 | aagatttagaagtttgagcacacccgcccactagggccccccatccaggggggcaacgggcaa |
| | | gcacttctgtttccccggtatgatctgataggctgtaaccacggctgaaacagagattatcgttatcc |
| | | gcttcactacttcgagaagcctagtaatgatgggtgaaattgaatccgttgatccggtgtctccccca |
| | | caccagaaactcatgatgagggttgccatcccggctacggcgacgtagcgggcatccctgcgct |
| | | ggcatgaggcctcttaggaggacggatgatatggatcttgtcgtgaagagcctattgagctagtgtc |
| | | gactcctccgcccccgtgaatgcggctaatcctaaccccggagcaggtgggtccaatccagggc |
| | | ctggcctgtcgtaatgcgtaagtctgggacggaaccgactactttcgggaaggcgtgtttccatttgt |
| | | tcattatttgtgtgtttatggtgacaactctgggtaaacgttctattgcgtttattgagagattcccaaca |
| | | attgaacaaacgagaactacctgttttattaaatttacacagagaagaattaca |
| 363 | Apodemus | attctcgggctacggccctggagccactccggctcctaaagatttagaagtttgagcacacccgcc |
| | Picornavirus | cactagggccccccatccaggggggcaacgggcaagcacttctgtttccccggtatgatctgata |
| | 5Δ201 | ggctgtaaccacggctgaaacagagattatcgttatccgcttcactacttcgagaagcctagtaatg |
| | | atgggtgaaattgaatccgttgatccggtgtctcccccacaccagaaactcatgatgagggttgcca |
| | | tcccggctacggcgacgtagcgggcatccctgcgctggcatgaggcctcttaggaggacggatg |
| | | atatggatcttgtcgtgaagagcctattgagctagtgtcgactcctccgcccccgtgaatgcggcta |
| | | atcctaaccccggagcaggtgggtccaatccagggcctggcctgtcgtaatgcgtaagtctggga |
| | | cggaaccgactactttcgggaaggcgtgtttccatttgttcattatttgtgtgtttatggtgacaactctg |
| | | ggtaaacgttctattgcgtttattgagagattcccaacaattgaacaaacgagaactacctgttttatta |
| | | aatttacacagagaagaattaca |
| 364 | Kobuvirus | tttcacaccctcttttccggtggtccggacccagaccaccgttactccattcagctacttcggtacctg |
| | SZAL6 | ttcggaggaattaaacgggcaccctacccaagggttacatgggaccatattcctcctcccctgtaac |
| | | tttaagttttgtgcccgtattcttgactccaggcggatgttgtgtcgcccgtcctgtgaacaaacagct |
| | | agacactttcctcccctccctctgggctgctccggcagtccactccctccccccagcgtaacatgcc |
| | | ccgctggagtgatgcacctggaagtcgtggacgtgggttagtaacttcggtgaaaacccactataa |
| | | tgacaactggttgacccccacactcaaaggactcgagtctttctcccttaaggctagcccggccac |
| | | atgaatttgcagctggcaactagtgagtccaccatgtcccgcaacctcggctgcggagtgctgttc |
| | | cccaagcgtatgccttccttctgtaagagtgcgcctggcaagcacatctgagaagtcgttccgctgc |
| | | gtcgtgccaacctggcgacaggtgacccagtgtgcgtagacttcttccggattcgtccggctcttct |
| | | ctaggaaacatgcgtgtaaggttcatgtgccaaagccctgcgcgcggtgttcttctactgccctagg |
| | | aatgtgccgcaggtacccctacttcggtagggatctgagcggtagctaattgtctacgggtagtttc |
| | | atttccatcttctcttcaggtcgacatc |
| 365 | Kobuvirus | ttgactccaggcggatgttgtgtcgcccgtcctgtgaacaaacagctagacactttcctcccctccct |
| | SZAL6 5Δ158 | ctgggctgctccggcagtccactccctccccccagcgtaacatgccccgctggagtgatgcacct |
| | | ggaagtcgtggacgtgggttagtaacttcggtgaaaacccactataatgacaactggttgaccccc |
| | | acactcaaaggactcgagtctttctcccttaaggctagcccggccacatgaatttgcagctggcaac |
| | | tagtgagtccaccatgtcccgcaacctcggctgcggagtgctgttccccaagcgtatgccttccttc |
| | | tgtaagagtgcgcctggcaagcacatctgagaagtcgttccgctgcgtcgtgccaacctggcgac |
| | | aggtgacccagtgtgcgtagacttcttccggattcgtccggctcttctctaggaaacatgcgtgtaa |
| | | ggttcatgtgccaaagccctgcgcgcggtgttcttctactgccctaggaatgtgccgcaggtaccc |
| | | ctacttcggtagggatctgagcggtagctaattgtctacgggtagtttcatttccatcttctcttcaggt |
| | | cgacatc |
| 366 | Kobuvirus | gaattaaacgggcaccctacccaagggttacatgggaccatattcctcctcccctgtaactttaagtt |
| | SZAL6 5Δ76 | ttgtgcccgtattcttgactccaggcggatgttgtgtcgcccgtcctgtgaacaaacagctagacact |
| | | ttcctcccctccctctgggctgctccggcagtccactccctccccccagcgtaacatgccccgctg |
| | | gagtgatgcacctggaagtcgtggacgtgggttagtaacttcggtgaaaacccactataatgacaa |
| | | ctggttgacccccacactcaaaggactcgagtctttctcccttaaggctagcccggccacatgaatt |
| | | tgcagctggcaactagtgagtccaccatgtcccgcaacctcggctgcggagtgctgttccccaag |
| | | cgtatgccttccttctgtaagagtgcgcctggcaagcacatctgagaagtcgttccgctgcgtcgtg |
| | | ccaacctggcgacaggtgacccagtgtgcgtagacttcttccggattcgtccggctcttctctagga |
| | | aacatgcgtgtaaggttcatgtgccaaagccctgcgcgcggtgttcttctactgccctaggaatgtg |
| | | ccgcaggtacccctacttcggtagggatctgagcggtagctaattgtctacgggtagtttcatttcca |
| | | tcttctcttcaggtcgacatc |
| 367 | Kobuvirus | tttcacaccctcttttccggtggtccggacccagaccaccgttactccattcagctacttcggtacctg |
| | SZAL6 3Δ37 | ttcggaggaattaaacgggcaccctacccaagggttacatgggaccatattcctcctcccctgtaac |
| | | tttaagttttgtgcccgtattcttgactccaggcggatgttgtgtcgcccgtcctgtgaacaaacagct |
| | | agacactttcctcccctccctctgggctgctccggcagtccactccctccccccagcgtaacatgcc |
| | | ccgctggagtgatgcacctggaagtcgtggacgtgggttagtaacttcggtgaaaacccactataa |
| | | tgacaactggttgacccccacactcaaaggactcgagtctttctcccttaaggctagcccggccac |
| | | atgaatttgcagctggcaactagtgagtccaccatgtcccgcaacctcggctgcggagtgctgttc |
| | | cccaagcgtatgccttccttctgtaagagtgcgcctggcaagcacatctgagaagtcgttccgctgc |
| | | gtcgtgccaacctggcgacaggtgacccagtgtgcgtagacttcttccggattcgtccggctcttct |
| | | ctaggaaacatgcgtgtaaggttcatgtgccaaagccctgcgcgcggtgttcttctactgccctagg |
| | | aatgtgccgcaggtacccctacttcggtagggatctgagcggtagctaattggacatc |
| 368 | Salivirus SZ1 | tctgtcctcaccccatcttcccttctttcctgcaccgttacgcttactcgcatgtgcattgagtggtgca |
| | | cgtgcttgaacaaacagctacactcacatgggggcgggttttcccgccctgcggcctctcgcgag |
| | | gcccacccctccccttcctcccataactacagtgctttggtaggtaagcatcctgatcccccgcgga |
| | | agctgctcacgtggcaactgtggggacccagacaggttatcaaaggcacccggtctttccgccttc |
| | | aggagtatccctgctagtgaattctagtagggctctgcttggtgccaacctcccccaaatgcgcgct |
| | | gcgggagtgctcttccccaactcaccctagtatcctctcatgtgtgtgcttggtcagcatatctgaga |
| | | cgatgttccgctgtcccagaccagtccagtaatggacgggccagtgtgcgtagtcgtcttccggctt |
| | | gtccggcgcatgtttggtgaaccggtggggtaaggttggtgtgcccaacgcccgtactttggtgat |
| | | acctcaagaccacccaggaatgccagggaggtaccccgcttcacagcgggatctgaccctggg |
| | | ctaattgtctacggtggttcttcttgcttccacttctttctactgttcatg |
| 369 | Crohivirus B | gtataagagacaggtgtttgccttgtcttcggactggcatcttgggaccaaccccccttttccccagc |
| | | catgggttaaatggcaataaaggacgtaacaactttgtaaccattaagctttgtaattttgtaaccact |
| | | aagctttgtgcacataatgtaaccatcaagcttgttagtcccagcaggaggtttgcatgcttgtagcc |
| | | gaaatggggctcgaccccccatagtaggatacttgattttgcattccattgtggacctgcaaactcta |
| | | cacatagaggctttgtcttgcatctaaacacctgagtacagtgtgtacctagaccctatagtacggga |
| | | ggaccgtttgtttcctcaataaccctacataataggctaggtgggcatgcccaatttgcaagatccca |
| | | gactgggggtcggtctgggcagggttagatccctgttagctactgcctgatagggtggtgctcaac |
| | | catgtgtagtttaaattgagctgttcatatacc |
| 370 | Crohivirus B | ccccccttttccccagccatgggttaaatggcaataaaggacgtaacaactttgtaaccattaagctt |
| | 5Δ51 | tgtaattttgtaaccactaagctttgtgcacataatgtaaccatcaagcttgttagtcccagcaggagg |
| | | tttgcatgcttgtagccgaaatggggctcgaccccccatagtaggatacttgattttgcattccattgt |
| | | ggacctgcaaactctacacatagaggctttgtcttgcatctaaacacctgagtacagtgtgtacctag |
| | | accctatagtacgggaggaccgtttgtttcctcaataaccctacataataggctaggtgggcatgcc |
| | | caatttgcaagatcccagactgggggtcggtctgggcagggttagatccctgttagctactgcctg |
| | | atagggtggtgctcaaccatgtgtagtttaaattgagctgttcatatacc |
| 371 | CVB3 | ttaaaacagcctgtgggttgatcccacccacagggcccattgggcgctagcactctggtatcacgg |
| | | tacctttgtgcgcctgttttataccccctcccccaactgtaacttagaagtaacacacaccgatcaac |
| | | agtcagcgtggcacaccagccacgttttgatcaagcacttctgttaccccggactgagtatcaatag |
| | | actgctcacgcggttgaaggagaaagcgttcgttatccggccaactacttcgaaaaacctagtaac |
| | | accgtggaagttgcagagtgtttcgctcagcactaccccagtgtagatcaggtcgatgagtcaccg |
| | | cattccccacgggcgaccgtggcggtggctgcgttggcggcctgcccatggggaaacccatgg |
| | | gacgctctaatacagacatggtgcgaagagtctattgagctagttggtagtcctccggcccctgaat |
| | | gcggctaatcctaactgcggagcacacaccctcaagccagagggcagtgtgtcgtaacgggcaa |
| | | ctctgcagcggaaccgactactttgggtgtccgtgtttcattttattcctatactggctgcttatggtga |
| | | caattgagagattgttaccatatagctattggattggccatccggtgaccaatagagctattatatatct |
| | | ctttgttgggtttataccacttagcttgaaagaggttaaaacattacaattcattgttaagttgaatacag |
| | | caaa |
| 372 | CVB3 3Δ91 | ttaaaacagcctgtgggttgatcccacccacagggcccattgggcgctagcactctggtatcacgg |
| | | tacctttgtgcgcctgttttataccccctcccccaactgtaacttagaagtaacacacaccgatcaac |
| | | agtcagcgtggcacaccagccacgttttgatcaagcacttctgttaccccggactgagtatcaatag |
| | | actgctcacgcggttgaaggagaaagcgttcgttatccggccaactacttcgaaaaacctagtaac |
| | | accgtggaagttgcagagtgtttcgctcagcactaccccagtgtagatcaggtcgatgagtcaccg |
| | | cattccccacgggcgaccgtggcggtggctgcgttggcggcctgcccatggggaaacccatgg |
| | | gacgctctaatacagacatggtgcgaagagtctattgagctagttggtagtcctccggcccctgaat |
| | | gcggctaatcctaactgcggagcacacaccctcaagccagagggcagtgtgtcgtaacgggcaa |
| | | ctctgcagcggaaccgactactttgggtgtccgtgtttcattttattcctatactggctgcttatggtga |
| | | caattgagagattgttaccatatagctattggattggccatccggtgaagcaaa |
| 373 | SAFV | cacttatttaattcggccttttgtgacaagcccctcggtgaaagaacctctctcttttcgacgtggttgg |
| | | aattgccatcatttccgacgaaagtgctatcatgcctccccgattatgtgatgttttctgccctgctgg |
| | | gcggagcattctcgggttgagaaaccttgaatctttttctttggaaccttggttcccccggtctaagcc |
| | | gcttggaatatgacagggttattttcttgatcttatttctacttttgcgggttctatccgtaaaaagggtac |
| | | gtgctgccccttccttctctggagaattcacacggcggtctttccgtctctcaacaagtgtgaatgca |
| | | gcatgccggaaacggtgaagaaaacagttttctgtggaaatttagagtgcacatcgaaacagctgt |
| | | agcgacctcacagtagcagcggactcccctcttggcgacaagagcctctgcggccaaaagcccc |
| | | gtggataagatccactgctgtgagcggtgcaaccccagcaccctggttcgatgatcattctctatgg |
| | | aaccagaaaatggttttctcaagccctccggtagagaagccaagaatgtcctgaaggtaccccgc |
| | | gtgcgggatctgatcaggagaccaattggcggtgctttacactgtcactttggtttaaaaattgtcac |
| | | agcttctccaaaccaagtggtcttggttttccaattttgttga |
| 374 | SAFV 5Δ46 | cctctctcttttcgacgtggttggaattgccatcatttccgacgaaagtgctatcatgcctccccgatta |
| | | tgtgatgttttctgccctgctgggcggagcattctcgggttgagaaaccttgaatctttttctttggaac |
| | | cttggttcccccggtctaagccgcttggaatatgacagggttattttcttgatcttatttctacttttgcgg |
| | | gttctatccgtaaaaagggtacgtgctgccccttccttctctggagaattcacacggcggtctttccgt |
| | | ctctcaacaagtgtgaatgcagcatgccggaaacggtgaagaaaacagttttctgtggaaatttaga |
| | | gtgcacatcgaaacagctgtagcgacctcacagtagcagcggactcccctcttggcgacaagag |
| | | cctctgcggccaaaagccccgtggataagatccactgctgtgagcggtgcaaccccagcaccct |
| | | ggttcgatgatcattctctatggaaccagaaaatggttttctcaagccctccggtagagaagccaag |
| | | aatgtcctgaaggtaccccgcgtgcgggatctgatcaggagaccaattggcggtgctttacactgt |
| | | cactttggtttaaaaattgtcacagcttctccaaaccaagtggtcttggttttccaattttgttga |
| 375 | SAFV 5Δ93 | gtgctatcatgcctccccgattatgtgatgttttctgccctgctgggcggagcattctcgggttgaga |
| | | aaccttgaatctttttctttggaaccttggttcccccggtctaagccgcttggaatatgacagggttattt |
| | | tcttgatcttatttctacttttgcgggttctatccgtaaaaagggtacgtgctgccccttccttctctgga |
| | | gaattcacacggcggtctttccgtctctcaacaagtgtgaatgcagcatgccggaaacggtgaaga |
| | | aaacagttttctgtggaaatttagagtgcacatcgaaacagctgtagcgacctcacagtagcagcg |
| | | gactcccctcttggcgacaagagcctctgcggccaaaagccccgtggataagatccactgctgtg |
| | | agcggtgcaaccccagcaccctggttcgatgatcattctctatggaaccagaaaatggttttctcaa |
| | | gccctccggtagagaagccaagaatgtcctgaaggtaccccgcgtgcgggatctgatcaggaga |
| | | ccaattggcggtgctttacactgtcactttggtttaaaaattgtcacagcttctccaaaccaagtggtct |
| | | tggttttccaattttgttga |
| 376 | SAFV 3Δ47 | cacttatttaattcggccttttgtgacaagcccctcggtgaaagaacctctctcttttcgacgtggttgg |
| | | aattgccatcatttccgacgaaagtgctatcatgcctccccgattatgtgatgttttctgccctgctgg |
| | | gcggagcattctcgggttgagaaaccttgaatctttttctttggaaccttggttcccccggtctaagcc |
| | | gcttggaatatgacagggttattttcttgatcttatttctacttttgcgggttctatccgtaaaaagggtac |
| | | gtgctgccccttccttctctggagaattcacacggcggtctttccgtctctcaacaagtgtgaatgca |
| | | gcatgccggaaacggtgaagaaaacagttttctgtggaaatttagagtgcacatcgaaacagctgt |
| | | agcgacctcacagtagcagcggactcccctcttggcgacaagagcctctgcggccaaaagcccc |
| | | gtggataagatccactgctgtgagcggtgcaaccccagcaccctggttcgatgatcattctctatgg |
| | | aaccagaaaatggttttctcaagccctccggtagagaagccaagaatgtcctgaaggtaccccgc |
| | | gtgcgggatctgatcaggagaccaattggcggtgctttacactgtcactttggtttaatgttga |
| 377 | SAFV Kozak | cacttatttaattcggccttttgtgacaagcccctcggtgaaagaacctctctcttttcgacgtggttgg |
| | | aattgccatcatttccgacgaaagtgctatcatgcctccccgattatgtgatgttttctgccctgctgg |
| | | gcggagcattctcgggttgagaaaccttgaatctttttctttggaaccttggttcccccggtctaagcc |
| | | gcttggaatatgacagggttattttcttgatcttatttctacttttgcgggttctatccgtaaaaagggtac |
| | | gtgctgccccttccttctctggagaattcacacggcggtctttccgtctctcaacaagtgtgaatgca |
| | | gcatgccggaaacggtgaagaaaacagttttctgtggaaatttagagtgcacatcgaaacagctgt |
| | | agcgacctcacagtagcagcggactcccctcttggcgacaagagcctctgcggccaaaagcccc |
| | | gtggataagatccactgctgtgagcggtgcaaccccagcaccctggttcgatgatcattctctatgg |
| | | aaccagaaaatggttttctcaagccctccggtagagaagccaagaatgtcctgaaggtaccccgc |
| | | gtgcgggatctgatcaggagaccaattggcggtgctttacactgtcactttggtttaaaaattgtcac |
| | | agcttctccaaaccaagtggtcttggttttccaattttgttgaccgcc |
| 378 | GLuc CK dCTG1 | gtggccacgcccgggccaccgatacttcccttcactccttcgggactgttggggaggaacacaac |
| | | agggctcccctgttttcccattccttcccccttttcccaaccccaaccgccgtatctggtggcggcaa |
| | | gacacacgggtctttccctctaaagcacaattgtgtgtgtgtcccaggtcctcctgcgtacggtgcg |
| | | ggagtgctcccacccaactgttgtaagcctgtccaacgcgtcgtcctggcaagactatgacgtcgc |
| | | atgttccgctgcggatgccgaccgggtaaccggttccccagtgtgtgtagtgcgatcttccaggtc |
| | | ctcctggttggcgttgtccagaaactgcttcaggtaagtggggtgtgcccaatccctacaaaggttg |
| | | attctttcaccaccttaggaatgctccggaggtaccccagcaacagctgggatctgaccggaggct |
| | | aattgtctacgggtggtgtttcctttttcttttcacacaactctacGTctgacaactcactgactatcca |
| | | cttgctctaaagtc |
| 379 | GLuc CK | gtggccacgcccgggccaccgatacttcccttcactccttcgggactgttggggaggaacacaac |
| | dCTG1_2 | agggctcccctgttttcccattccttcccccttttcccaaccccaaccgccgtatctggtggcggcaa |
| | | gacacacgggtctttccctctaaagcacaattgtgtgtgtgtcccaggtcctcctgcgtacggtgcg |
| | | ggagtgctcccacccaactgttgtaagcctgtccaacgcgtcgtcctggcaagactatgacgtcgc |
| | | atgttccgctgcggatgccgaccgggtaaccggttccccagtgtgtgtagtgcgatcttccaggtc |
| | | ctcctggttggcgttgtccagaaactgcttcaggtaagtggggtgtgcccaatccctacaaaggttg |
| | | attctttcaccaccttaggaatgctccggaggtaccccagcaacagctgggatctgaccggaggct |
| | | aattgtctacgggtggtgtttcctttttcttttcacacaactctacGTcGTacaactcactgactatcc |
| | | acttgctctaaagtc |
| 380 | GLuc CK | gtggccacgcccgggccaccgatacttcccttcactccttcgggactgttggggaggaacacaac |
| | dCTG1_2_3 | agggctcccctgttttcccattccttcccccttttcccaaccccaaccgccgtatctggtggcggcaa |
| | | gacacacgggtctttccctctaaagcacaattgtgtgtgtgtcccaggtcctcctgcgtacggtgcg |
| | | ggagtgctcccacccaactgttgtaagcctgtccaacgcgtcgtcctggcaagactatgacgtcgc |
| | | atgttccgctgcggatgccgaccgggtaaccggttccccagtgtgtgtagtgcgatcttccaggtc |
| | | ctcctggttggcgttgtccagaaactgcttcaggtaagtggggtgtgcccaatccctacaaaggttg |
| | | attctttcaccaccttaggaatgctccggaggtaccccagcaacagctgggatctgaccggaggct |
| | | aattgtctacgggtggtgtttcctttttcttttcacacaactctacGTcGTacaactcacGTactat |
| | | ccacttgctctaaagtc |
| 381 | GLuc CK dAll | gtggccacgcccgggccaccgatacttcccttcactccttcgggactgttggggaggaacacaac |
| | | agggctcccctgttttcccattccttcccccttttcccaaccccaaccgccgtatctggtggcggcaa |
| | | gacacacgggtctttccctctaaagcacaattgtgtgtgtgtcccaggtcctcctgcgtacggtgcg |
| | | ggagtgctcccacccaactgttgtaagcctgtccaacgcgtcgtcctggcaagactatgacgtcgc |
| | | atgttccgctgcggatgccgaccgggtaaccggttccccagtgtgtgtagtgcgatcttccaggtc |
| | | ctcctggttggcgttgtccagaaactgcttcaggtaagtggggtgtgcccaatccctacaaaggttg |
| | | attctttcaccaccttaggaatgctccggaggtaccccagcaacagctgggatctgaccggaggct |
| | | aattgtctacgggtggtgtttcctttttcttttcacacaactctacGTcGTacaactcacGTactaC |
| | | TcactGTctctaaagtc |
| 382 | CK SZ1-L1S | gggggtggggggggcctcggccccctcaccctcttttccggtggccacgcccgggccaccgat |
| | | acttcccttcactccttcgggactgttggggaggaacacaacagggctcccctgttttcccattcctt |
| | | cccccttttcccaaccccaaccgccgtatctggtggcggcaagacacacgggtctttccctctaaa |
| | | gcacaattgtgtgtgtgtcccaggtcctcctgcgtacggtgcgggagtgctcccacccaactgttgt |
| | | aagcctgtccaacgcgtcgtcctggcaagactatgacgtcgcatgttccgctgcggatgccgacc |
| | | gggtaaccggttccccagtgtgtgtagtgcgatcttccaggtcctcctggttggcgttgtccagaaa |
| | | ctgcttcaggtaagtggggtgtgcccaatccctacaaaggttgaaccacccaggaatgccaggga |
| | | ggtaccccgcttcacagcgggatctgaccctgggctaattgtctacggtggttcttcttgcttccactt |
| | | ctttctactgttcgccacc |
| 383 | CK Aichi Scan | gggggtggggggggcctcggccccctcaccctcttttccggtggccacgcccgggccaccgat |
| | (AV-S) | acttcccttcactccttcgggactgttggggaggaacacaacagggctcccctgttttcccattcctt |
| | | cccccttttcccaaccccaaccgccgtatctggtggcggcaagacacacgggtctttccctctaaa |
| | | gcacaattgtgtgtgtgtcccaggtcctcctgcgtacggtgcgggagtgctcccacccaactgttgt |
| | | aagcctgtccaacgcgtcgtcctggcaagactatgacgtcgcatgttccgctgcggatgccgacc |
| | | gggtaaccggttccccagtgtgtgtagtgcgatcttccaggtcctcctggttggcgttgtccagaaa |
| | | ctgcttcaggtaagtggggtgtgcccaatccctacaaaggttgattctttcaccaccttaggaatgct |
| | | ccggaggtaccccagcaacagctgggatctgaccggaggctaattgtctacgggtggtgtttcatt |
| | | tccaatccttttatgtcggagtc |
| 384 | CK Aichi Loop | gggggtggggggggcctcggccccctcaccctcttttccggtggccacgcccgggccaccgat |
| | (AV-L1) | acttcccttcactccttcgggactgttggggaggaacacaacagggctcccctgttttcccattcctt |
| | | cccccttttcccaaccccaaccgccgtatctggtggcggcaagacacacgggtctttccctctaaa |
| | | gcacaattgtgtgtgtgtcccaggtcctcctgcgtacggtgcgggagtgctcccacccaactgttgt |
| | | aagcctgtccaacgcgtcgtcctggcaagactatgacgtcgcatgttccgctgcggatgccgacc |
| | | gggtaaccggttccccagtgtgtgtagtgcgatcttccaggtcctcctggttggcgttgtccagaaa |
| | | ctgcttcaggtaagtggggtgtgcccaatccctacaaaggttgaactgccctaggaatgccaggca |
| | | ggtaccccacctccgggtgggatctgagcctgggctaattgtctacgggtagttttcctttttcttttca |
| | | cacaactctactgctgacaactcactgactatccacttgctctcttgtgcctttctgctctggttcaagtt |
| | | ccttgattgtttttgactgcttttcactgcttttcttctcacaatccttgctcagttcaaagtc |
| 385 | CK SZ1-L2 | gggggtggggggggcctcggccccctcaccctcttttccggtggccacgcccgggccaccgat |
| | | acttcccttcactccttcgggactgttggggaggaacacaacagggctcccctgttttcccattcctt |
| | | cccccttttcccaaccccaaccgccgtatctggtggcggcaagacacacgggtctttccctctaaa |
| | | gcacaattgtgtgtgtgtcccaggtcctcctgcgtacggtgcgggagtgctcccacccaactgttgt |
| | | aagcctgtccaacgcctgatcccccgcggaagctgctcacgtggcaactgtggggacccagaca |
| | | ggttatcaaaggcacccggtctttccgccttcaggagtatccctgctagtgaattctagtagggctct |
| | | gcttgcgttgtccagaaactgcttcaggtaagtggggtgtgcccaatccctacaaaggttgattcttt |
| | | caccaccttaggaatgctccggaggtaccccagcaacagctgggatctgaccggaggctaattgt |
| | | ctacgggtggtgtttcctttttcttttcacacaactctactgctgacaactcactgactatccacttgctct |
| | | cttgtgcctttctgctctggttcaagttccttgattgtttttgactgcttttcactgcttttcttctcacaatcc |
| | | ttgctcagttcaaagtc |
| 386 | CK Aichi | gggggtggggggggcctcggccccctcaccctcttttccggtggccacgcccgggccaccgat |
| | TriLoop (AV-L2) | acttcccttcactccttcgggactgttggggaggaacacaacagggctcccctgttttcccattcctt |
| | | cccccttttcccaaccccaaccgccgtatctggtggcggcaagacacacgggtctttccctctaaa |
| | | gcacaattgtgtgtgtgtcccaggtcctcctgcgtacggtgcgggagtgctcccacccaactgttgt |
| | | aagcctgtccaacgcatgtgcctggcaagcatatctgagaaggtgttccgctgtggctgccaacct |
| | | ggtgacaggtgccccagtgtgcgtaaccttcttccgtctccggacggtgcgttgtccagaaactgct |
| | | tcaggtaagtggggtgtgcccaatccctacaaaggttgattctttcaccaccttaggaatgctccgg |
| | | aggtaccccagcaacagctgggatctgaccggaggctaattgtctacgggtggtgtttcctttttcttt |
| | | tcacacaactctactgctgacaactcactgactatccacttgctctcttgtgcctttctgctctggttcaa |
| | | gttccttgattgtttttgactgcttttcactgcttttcttctcacaatccttgctcagttcaaagtc |
| 387 | CK Scan | gggggtggggggggcctcggccccctcaccctcttttccggtggccacgcccgggccaccgat |
| | Deletion (ΔS) | acttcccttcactccttcgggactgttggggaggaacacaacagggctcccctgttttcccattcctt |
| | | cccccttttcccaaccccaaccgccgtatctggtggcggcaagacacacgggtctttccctctaaa |
| | | gcacaattgtgtgtgtgtcccaggtcctcctgcgtacggtgcgggagtgctcccacccaactgttgt |
| | | aagcctgtccaacgcgtcgtcctggcaagactatgacgtcgcatgttccgctgcggatgccgacc |
| | | gggtaaccggttccccagtgtgtgtagtgcgatcttccaggtcctcctggttggcgttgtccagaaa |
| | | ctgcttcaggtaagtggggtgtgcccaatccctacaaaggttgattctttcaccaccttaggaatgct |
| | | ccggaggtaccccagcaacagctgggatctgaccggaggctaattgtctacgggtggtg |
| 388 | CK Loop | gggggtggggggggcctcggccccctcaccctcttttccggtggccacgcccgggccaccgat |
| | Deletion (ΔL1) | acttcccttcactccttcgggactgttggggaggaacacaacagggctcccctgttttcccattcctt |
| | | cccccttttcccaaccccaaccgccgtatctggtggcggcaagacacacgggtctttccctctaaa |
| | | gcacaattgtgtgtgtgtcccaggtcctcctgcgtacggtgcgggagtgctcccacccaactgttgt |
| | | aagcctgtccaacgcgtcgtcctggcaagactatgacgtcgcatgttccgctgcggatgccgacc |
| | | gggtaaccggttccccagtgtgtgtagtgcgatcttccaggtcctcctggttggcgttgtccagaaa |
| | | ctgcttcaggtaagtggggtgtgcccaatccctacaaaggttgatttcctttttcttttcacacaactct |
| | | actgctgacaactcactgactatccacttgctctcttgtgcctttctgctctggttcaagttccttgattgt |
| | | ttttgactgcttttcactgcttttcttctcacaatccttgctcagttcaaagtc |
| 389 | CK Triloop | gggggtggggggggcctcggccccctcaccctcttttccggtggccacgcccgggccaccgat |
| | Deletion (ΔL2) | acttcccttcactccttcgggactgttggggaggaacacaacagggctcccctgttttcccattcctt |
| | | cccccttttcccaaccccaaccgccgtatctggtggcggcaagacacacgggtctttccctctaaa |
| | | gcacaattgtgtgtgtgtcccaggtcctcctgcgtacggtgcgggagtgctcccacccaactgttgt |
| | | aagcctgtccaacgcgcgttgtccagaaactgcttcaggtaagtggggtgtgcccaatccctacaa |
| | | aggttgattctttcaccaccttaggaatgctccggaggtaccccagcaacagctgggatctgaccg |
| | | gaggctaattgtctacgggtggtgtttcctttttcttttcacacaactctactgctgacaactcactgact |
| | | atccacttgctctcttgtgcctttctgctctggttcaagttccttgattgtttttgactgcttttcactgctttt |
| | | cttctcacaatccttgctcagttcaaagtc |
| 413 | RhPV | gataaaagaacctataatcccttcgcacaccgcgtcacaccgcgctatatgctgctcattaggaatt |
| | | acggctccttttttgtggatacaatctcttgtatacgatatacttattgttaatttcattgacctttacgcaa |
| | | tcctgcgtaaatgctggtatagggtgtacttcggatttccgagcctatattggttttgaaaggaccttta |
| | | agtccctactatactacattgtactagcgtaggccacgtaggcccgtaagatattataactattttatta |
| | | tattttattcaccccccacattaatcccagttaaagctttataactataagtaagccgtgccgaaacgtt |
| | | aatcggtcgctagttgcgtaacaactgttagtttaattttccaaaatttatttttcacaatttttagttaaga |
| | | ttttagcttgccttaagcagtctttatatcttctgtatattattttaaagtttataggagcaaagttcgcttta |
| | | ctcgcaatagctattttatttattttaggaatattatcacctcgtaattatttaattataacattagctttatct |
| | | atttata |
| 414 | Halastavi arva | cttgattctaaccttgccgtatggtgccctaacgggttcatttaatcatgcgatgagggttgctatacc |
| | (1x mut) | gcatccattctaaggcgattcaatgcttcatttaggaattttgttgacgattaaaaggtacccccacaa |
| | | aaacaaaaccaatcttacttgattttcgttttaactgaccactgcgatcccaaattttcgccttcttatca |
| | | aagtatgttgtgttctttgggtgtacaacctgagaacttgtctacaactacatattactcgaggaagaa |
| | | attcggtttaagccgtgccttctcacgtttagtatatctatctGgacacaccttcttcatcttctaatccc |
| | | catctagtctcctgatcagagacgtcgttattaacaaataaccccccttgttaataagagacaaagta |
| | | caatcaagctaagttctcttggagttcctgtaggaacttagccattgtgatagagtcataagtctatgt |
| | | gcatagacagctctagctcaccatttccttcccaacccatcttttcatcagcttaactctatgaatccga |
| | | tgcaaaaaccattctaacatcttatggtgctttccaagccaaatgagagctcactcttttgagccgcta |
| | | tttaatggacaataaacgttttatagtgtacatcatattgtaaaaacaaa |
| 415 | Oscivirus | cctcggtccctctttccgtcgccgcccacgacgttaaatgcggtgttgtggtgcttaggtgccacac |
| | | cactgctatttgggtccccctttcccctatatatgtttgtttgtttatttcaatttcttgaggattggcacctc |
| | | cttatgccaaatctaaatcgtggaggatcccaggctttctggtctttaacagaactccacgtccaggt |
| | | catagaaactggttggtaggctgcctgagtagtccatttgctagtagtcccttgtgaacagggtggct |
| | | cccgtttactgctggtattcccggtgtaggtcgccatggtggtaacaccatcctgcattgtgtgtgaa |
| | | ccagtaccgcaaggatagcaaggtatgaacacttgtggacgaaatggtaagtgatcaattcactttc |
| | | atggccggaaggtcacgtggcaatcatgccacccaggtaccctcctctgggaggatctgagggt |
| | | gggctaagcagaccctgccatgtggctgaacttttcccttattgttttactttgtaacatttatagttgtgt |
| | | tagtgatttgtgtgttgtgcccttgtgagctatatccagtataagttcgcagctagaagttaatccttcg |
| | | acatcggctgtattggaa |
| 416 | Cadicivirus B | caccaacccttgacctgtaatgtcagtggacagagtgctcctctgttcccggttaccgtgttccagg |
| | | acacgattgtaatcctgcgcctcaccagcgctgcgtgcacgtctgcataaggaaacgtgccttccc |
| | | catgtctctatcaattctttggtgagtgaccgccctagttgctcatcctatgggattcttctctcatgggt |
| | | tctttgtggcatgcgaatgtcaccttaattggaggtctttaattagatatcctttcttcatctttgatatgag |
| | | tgtcggaatttgattcctagtctctgcaaaacaaccccacttgatgaattcaacttttcaaccgcacaa |
| | | acataatcaggtttttaaattgaatgtttctaaattctaaatttagtttatttaagtagtttgccatcttgact |
| | | cgatgtaaaattgtcatacaagtcttcttttcttttctttacactttgaagtttgcacttagcagtcgttctg |
| | | cacagctttcgagttttgtttgatcgacatcgcaacttccacccacctctctttttctagtgttgaatgcg |
| | | gctaatcctaacccgagagcaataaacccaggtttattgtcgtaacgcgcaagtcttggacggaac |
| | | cgactatacacacacctctttaccctttagtacacccttggtacg |
| 417 | PSIV (2x mut for | gcaaaatgggtacgtagttaaccactgcgtatcaggattgcaggccacgaagggtatttgcatatct |
| | Xba1) | ttctatgcggtattacggcttaaaacccgttgtatcttgtTgtttgactgcctgtatcactagtggccatt |
| | | ttatttaggttagagacccctgatagtaggagagttacaaactctttaaaaattgttgaccccggaaa |
| | | agatggtgacccctgtaagtagttgatcAagaagatctatgcgctggcatagtaatccagtgtttcc |
| | | tgttttaggatgacctctgaaagtagatgaccgtggaaagtcacgtagtgccccaataagcacgttt |
| | | gggcagcgtgcgctatcacaaggcttgatctccgaggagccccttgttttagctggctggaagcca |
| | | atgatcttaagtagataagtgctgttgcttgtagttcaacagaaagctttgagtacgtctttcttgcgag |
| | | aaagaacacatgcattcttatgctctcaattctattatttttattttgggcgaaaggaaagctctcacgc |
| | | gagtacgaatagccaaccctttat |
| 418 | PSIV IGR | GCTGACTATGTGATCTTATTAAAATTAGGTTAAATTTCGAG |
| | | GTTAAAAATAGTTTTAATATTGCTATAGTCTTAGAGGTCTT |
| | | GTATATTTATACTTACCACACAAGATGGACCGGAGCAGCCC |
| | | TCCAATATCTAGTGTACCCTCG |
| 419 | PV Mahoney | ATGAGTCTGGACATCCCTCACCGGTGACGGTGGTCCAGGCT |
| | | GCGTTGGCGGCCTACCTATGGCTAACGCCATGGGACGCTAG |
| | | TTGTGAACAAGGTGTGAAGAGCCTATTGAGCTACATAAGA |
| | | ATCCTCCGGCCCCTGAATGCGGCTAATCCCAACCTCGGAGC |
| | | AGGTGGTCACAAACCAGTGATTGGCCTGTCGTAACGCGCA |
| | | AGTCCGTGGCGGAACCGACTACTTTGGGTGTCCGTGTTTCC |
| | | TTTTATTTTATTGTGGCTGCTTATGGTGACAATCACAGATTG |
| | | TTATCATAAAGCGAATTGGATTGGCC |
| 420 | REV A | GGGGTCGCCGTCCTACACATTGTTGTGACGTGCGGCCCAGA |
| | | TTCGAATCTGTAATAAAAGCTTTTTCTTCTATATCCTCAGAT |
| | | TGGCAGTGAGAGGAGATTTTGTTCGTGGTGTTGGCTGGCCT |
| | | ACTGGGTGGGGTAGGGATCCGGACTGAATCCGTAGTATTTC |
| | | GGTACAACATTTGGGGGCTCGTCCGGGATTCCTCCCCATCG |
| | | GCAGAGGTGCCTACTGTTTCTTCGAACTCCGGCGCCGGTAA |
| | | GTAAGTACTTGATTTTGGTACCTCGCGAGGGTTTGGGAGGA |
| | | TCGGAGTGGCGGGACGCTGCCGGGAAGCTCCACCTCCGCT |
| | | CAGCAGGGGACGCCCTGGTCTGAGCTCTGTGGTATCTGATT |
| | | GTTGTTGAACCGTCTCTAAGACGGTGATACTATAAGTCGTG |
| | | GTTTGTGTGTTTGTTTGTTACCTTGTGTTTGTTCGTCACTTGT |
| | | CGACAGCGCCCTGCGAATTGGTGTACCCACACCGCGCGGCT |
| | | TGCGAATAATACTTTGGAGAGTCTTTTGCCTCCAGTGTCTTC |
| | | CGTTTGTACTCGTCCTCCTCTCCCTCTCCGGCCGGGATGGG |
| 421 | Tropivirus A | tgtcgcatgttgccaacatcaaaattctgggagagtcgcgaactccttaacactgccttgcctcgac |
| | | ggagccgttgttatagtgtcgacgggatacaaacattaaactaaacccacttgcctcgacggaacc |
| | | ccttaccttttattttattttatagtatgaaagtgaatcttgtatgaatgttcatagaaaactgcaaatgagt |
| | | accacgtctaacatgagagaatgatactggagaaatccaagtttagaagtcactacgaatcccagc |
| | | ggaaacaagggaattctgagcttctaataggcgtttaagactatttgcaaaattctggtgcgtaagtg |
| | | atattttcattgcgtagaacgctggtaaccactccggctagtataagcattgttagtcacttattatgaa |
| | | actccacactatcctttctggagaagcacacaaacttacatggtaaagctagaccattatcttaagcg |
| | | gtgagtacactgcaaccttgtaacaatgcttgtatgactactttttgtatatcttgagcaatattgttgag |
| | | gtggacatgtccaaaggtaatgttgttgggaatggaggggtccattttcccgtgcacgtagtgtact |
| | | agtattgggtgatagccttgcggcggatcaaccatgtattttaatccgttgactttcac |
| 422 | Symapivirus A | ttgggaaatccccaatgcttctttcaacaccgcctgactatgcggtggcgcttcggctcaaacaact |
| | | agtcacttccccctcttaactactacccaagacttctaactacccttacctacttatttgtctaaatttcaa |
| | | acttttattctcacgcgtcttataaacatcttttctatttgttatggtatgttttgtgatttgtgtggtgtatttc |
| | | atttaatgggatctagtggaccgtgccccggttgggtatccgctccctttaaatgtttgcaagcactct |
| | | tgacattataacctatcatttagtttacttgtttgtatgatcgtatttctgaatcgtaacatttatgcaattctt |
| | | tctcgccgagacttgtctaggagataaagttcctgcatatttagtgttacggttgtataatggagactta |
| | | gatagcttcacactgaggacgctttttcgctatccttttgacctgattcaggccagtgtggagttaatg |
| | | attgtatggatgggccctacaatttgtctaagacttggtgatagcctcgcggccgctcgccatttata |
| | | caactgaatagcggttgaaactctct |
| 423 | Sakobuvirus A | tcacgcgcttttccggtggtcacccaccgttagggagcgccagcgttcgcgcttccgctaccaggt |
| | FFUP1 (1x mut) | gacacactcctttcccctcccccattcccgttcccatcctctggactggtttctcctcacgattgacca |
| | | gcagctgggagctgttaccagacgttggacagtaagtcccggatgcactatagggctggtggcta |
| | | gtgcttggtaagcactcaacgccatacctaatgtgtacctcggcttgccctcctggtcgtggtgacc |
| | | ggctgtttctcttcccttggctcCagacgggctggtgtcctaccaccaccgttgcatgcagacctcc |
| | | ccctgcgcactcgaacgccctgtcccagcagggttagtatgtgctgtgcagatctgcatgtgacac |
| | | cccatccactggtagagcaggaagttgccctagctaacgcggcaagtattactttccgctacacgt |
| | | ccttgagattcctcggacctctggaactagggtgactgtgggcttgggaaaacccaccttggtcctg |
| | | tactgcctgatagggtcgcggctggccgaccagtggatgtagccagttgttttgggat |
| 424 | Rosavirus C | cagggagatctccatgaataatcttttccaccctctttagcgtctatgctattgaggacgggttggag |
| | NFSM6F | ccccgttgacccagcgtcagagtgtgtcggtagcaggctttctgctctcgccccatgccggccaca |
| | | cctcccattagtgatgtgaaggttgtaagttacatgtgaaaaggtttctaataattgagctgaatgtag |
| | | cgattacctaaggtgagcggattcccccacgtggtaacacgtgcctctcaggccaaaagccaagg |
| | | tgttaaaagcaccccttaggtaggccactaccccgtggcctcagttctcttagaagattcacttagta |
| | | gtgtgtgcactggcaactcttaagcagagctagtgagtgggctaaggatgccctgaaggtacccg |
| | | caggtaacgttaagacactgtggatctgatcaggggctcgagtgctgaagctttacagaggtagct |
| | | cgagttaaaaaacgtctatgcccctcccccacgggagtgggggttcccccacaccaattttagatt |
| | | gcact |
| 425 | Rosavirus 2 | ccaggcatggcgttaaacatgcattcccttcccctagtaacctcccttcgccccttccccacgttgta |
| | GA7403 | ccccctccgagatggctgctaaggcgcttgctgctacagcagtctcgtgtttcgggtgttataagtg |
| | | ctttcttttccactccactccctgcctatggggagcggaacggccttgtctcggtcgttgcttcttgca |
| | | gatcttcacccctccaggctttctggactcgccaggggtggagtagtaggcgcactgtctaagtga |
| | | aggtagcagtgttgttggcgaagagttgtggacctactttgagtttgtagcgatcatccagagctag |
| | | cggatctccccacgcggtaacgcgtgcctctaggcccaaaaggcacggtgttcacagcacccttt |
| | | ggatggcgggggtgcccccctccgcacttaaagtagaaaaacagcttagtagtcaaataacatgg |
| | | ctttcctcaagcattcagtgctacatgggactgaaggatgcccagaaggtacccgcaggcaacga |
| | | taagctcactgtggatctgatctggggccctgggccaggtgctatacacctggttaaaaccaaatct |
| | | ggtagtcagggttaaaaaacgtctaagtcccacccccccggggacggggggttcccttaaaccct |
| | | caactgacacc |
| 426 | Rhimavirus A | cgaattccggacatctcctttcgggggcgagcgtcaccgtgcccctcatggaggcaactgtgcctc |
| | | taatcggtgacccactgagaaaattttctttctacgtggctaaacaatgcaactttataataacacaaa |
| | | tttaatgcttaatcttaacaccaaagatttgaacatatgtttggaaagtggcacacttcaaacattgcat |
| | | agttgctaggggtgaagtccctttaaggggttgcagaggatctttcctctttatgagcggctaggagt |
| | | atcttcttgatattatgtggtcgtgcaactcacttcccagatgtatgacggtgtactaagcgattggaa |
| | | ctagtcataacctctttgaattttggtattgcgagtctagcagggggatatttaccgctaaagggtgac |
| | | acactcgtgagggtggcctttggtgtgtgtatatttattccgcccatcttgcatggggtgctaaaattct |
| | | aatgctgtgaaataaccattttctgaatacattctctacatttggagtcaaatatgaggaatgccactca |
| | | ggtacccttgacatgatcttggatctgagagtgggctaattatctaattatttggcgactttctaaaatct |
| | | tctgtttttagtggtgacaatttatggttataaa |
| 427 | Rafivirus | gtgtccgggaagcgactcaagcttttgactgagtctctacaccttcatccgtaacatctttaagtttatg |
| | LPXYC222841 | tgcctatggacctctagtgcactgccatcaccgggggtgtattggactggtttttccacaatccattca |
| | | tcctgaggaattttggctttgttactaggatggtcccaccacacgcttatctgtgcctattgtgtcaacc |
| | | atgttcttaagtagttgtgcccgtgggtgagtagataaccacaacaatccgataaagcatctcgcaa |
| | | ggatgtgagtaatggagtgtatgtgctacagagacccacaacctgaaccaagagagacacagtg |
| | | aggattgtaaagggggaactctttgaaagggcatgtcccgcaattcctactgactgacaccgggg |
| | | gttggtgtcggtggattttagcaaatcctgttactgggtgatagccttgtgcacttcacttggttcttgta |
| | | taagtgctgta |
| 428 | Rafivirus | tgcgaatttattcgcacagtctcttttcccccatcttgtgtgtgtgatggggtaagccgcagagtaata |
| | WHWGGF74766 | cctactctgctgcaaacacactcactcttttctatctactttatatcatgtaataataagtagggaacata |
| | | ttcaattcatattgttcatctcactgaacccgcatgaaggactgcattgcatatcctggacgaagtgac |
| | | gtggaatatttggacatttatggattggacaccattacgctttgtgcctctacggagatgtaaccataa |
| | | tcttaagtagtagtaccccagcacaagaggataaagtggcatacacgacaacgggtgttgctcgc |
| | | accttagtaatgtggatgttcacccttggagcgtgctgaaactctgtgggtaaagacacacattagta |
| | | caaatgtgggggaactcactgaaagggcatgtcccgtgtactggtgtgccggaaagtgggggtc |
| | | gctttctggagaacttagtagttcttgttattgggtgatagccttgcggcggatcaactcacagttttaa |
| | | tccgttgttttgcat |
| 429 | Poecivirus | actacacaatcgcaacacgcgcaagtttgtagtttgattggcgtgcaaatgtcaaatcaagcatata |
| | BCCH-449 | acacaatttggtggctgttggtgtttgttataggaattttggttgtgttgaaattgtggatgtgtaggaaa |
| | | tatgcacaattacgtcagcgtcaggagttttataacctggcgcaacaccaaaatggtcttcgcgcttt |
| | | aacatcaccagcgaggtgtaaacaaattgaagttgaattagatcgtgtataggccagggaaccatc |
| | | cctcccaacgccacatcttgtggggaagttgggataatggtgggtctatatgaattggtctgtagac |
| | | ccacagtgaagagtgaatagtatgcttgcggttccatttgttaatggtctagcatgggtgggggcgg |
| | | caaccccgtgaggggttccccactggccaaaagcccaggggttagtcatttcaaccaaggaagct |
| | | ggtaacctggtgacctgaacttgagtggtgagacccccttgctagagtgtgtaaaccgattgtaagc |
| | | attttgtttgcttagtatctgtggtataagcagtcaattttgtataggctcaaggctgtggtagttagtag |
| | | atgcccggaaggttattactgatccggggaccgtgactatacattaggtaaaccggtttaaaaacc |
| 430 | Megirivirus A | ttcgggacactggatgggcgacttggtggggctgccactctatcttgacctttcgttactgactttcg |
| | LY | gatctctgactcctccttgtctcttgcgtttggtccacggacggactaattggaatgtttactggctaag |
| | | cctcgttctgaaataccctagccaatgggttgtagtaggatcctggtgtttccattaaacctcttccga |
| | | ccatagtagctagagttatggctgtgtaggatgtgggtaagaccgctttttgcgtatctcccacaaga |
| | | caccggattatggatgtgtccgctggataaggctcgaaacctcccaactgaaggtggtgctgaaat |
| | | attgcaagcctaggttgtgtagaggcaagtagatgcctgccgcgacattcgtcttccgcccttttgg |
| | | gttagtagtgtacctacatggacgtggggctgggaatccccaccttgcataacactggttgatagac |
| | | ctgcggctggtcaagttactatggtataaccagttgaaatggct |
| 431 | Megirivirus E | gcttggcaacctcatatcgttactctgccgaccagtctgggtcgtgtggccacacaatgggattcgtt |
| | | ctgttgtgtagagtcacatggcattactgggctgatcggtggggatccgttgccacccctaaaccct |
| | | tacatttactggactgcttttcttggccccggaatgattcgctcacccgcgatgaggactgttgttctta |
| | | ttatggcaggattacgcgtctggtccgcgtaaggactaattcctatgtttatacgttactaccttgttct |
| | | gaacggtgggcgccaccccgcctagtaggatcctggcttatcgtgtagacctctagggaccacatt |
| | | agctagagtgtaggctgctatggatggagtagtgacccctttttgggtatcactctctaagactccgg |
| | | aatgtgtcatagtacgctggaaatccttacttgtttttccatgagggggaggtggtgctgaaatattgc |
| | | aagccacccctcggttaaaacagtttggtgccgcttatgccatattaccgccccttgtagttgggctg |
| | | tttttgcagctccgggttagtagagtaccatagtggacgcggtgttgggaatcaccgccttggctgc |
| | | acactgcttgatagagctgcggctggtcaagctaattgtggtataaccagttgatttggcat |
| 432 | Megirivirus C | ttcccgaccggtctggcaaaccggacggttatcctggttagatgtctgatggttgctggaacgtggt |
| | | ggctactgctgccaccttctggcttcctttaatgggcatctagctgggttctttgccacaatccatctta |
| | | ctctcttacccattttctattacccagacttgttgttaactggtaaagttgacctactggcttcgttttgag |
| | | actattctggtgttggtggacactctttccacaagtagattgtatggagttcatgctcgttttgaaccgg |
| | | gaatggcacaacccgtagtaggatcttgcctctgccatactaatctgcgcctgttgcttttagactatg |
| | | ggctgctaaggatgacattggaacccctttttggatattccatgtcaagtcaactgtttcatctggtgta |
| | | cgctggaaatccttgttccgaggtcttgtctggaggtggtgctgaaatattgcaagccacaggcagt |
| | | tccttggacttggtgccgctatcagatgctacaccctctatgggcaaatgttgaaccttagtggacgc |
| | | gtgagatgggaatccacgccggccatagactggctgataagctcgcggctgatcgagttgcaaca |
| | | gtaatcagttgatttgccact |
| 433 | Ludopivirus | tagacccccacctagcccttttccccgtcagtggggggcttactcactgggcatctgttaatctggc |
| | | ctaactagattgacaccactcccttggaacgtaactccacgctaactcactggctctacgcacagac |
| | | acacggtctttctgctatccccggggaagataccagatggcgaccggctgtcccagcggcctagt |
| | | agctactcgggttgagtacccaccacggttttgacgcctgctaaaattcaagagacagaggtaggg |
| | | gtgcttagtgtgtgggggaagttcccacaagcgaggcaaagcattgctccctcgcgtcaccgggt |
| | | gcaaggtaaattggctggacttccgctctacccttgctactcgccctcttcggagggttcgaagtga |
| | | cactaggtatacgcatggttgggaaaccatgcctggcctactactgggtgatagcctggcggcgg |
| | | gtccgtctcttggcttatacccgttgatttgggat |
| 434 | Livupivirus | tatctacatggggatccaggctgtatggaatgtctgtcttaacaagcactataccagaaagatccac |
| | | ccaaagtggtgggactgggactgtgaggtgagaaatcccgaaaccagccttctcaagcgtcgga |
| | | cgatctttctgttttagtgaacaccttgccttttaaatggatgacaacaccccttcagcaaatcgcaatc |
| | | tgaaatcccaaaagactgtttagccgaactctggtaatcactccggagaagtaggatacgcagccc |
| | | ctgtggactcttgatttcaggactcaaggtagctagagctggaacttcatggaatgacaaaggaata |
| | | tatgcacattgtgcgctttcctggccttgtagcccgtcgtgaggatatgtcgttgggaatcgacatctt |
| | | agtccagtactgcttgatagagtgtcggctggcacagttacctgagaataagtcagttgtacttaaca |
| | | tgaacaaaaaaaataactaccacaactaccacaatctaccaatacttgaattatgctgaatctcgtac |
| | | agtaaaaacgttccgtggaaggacaagtattgaagtgcggttacatcatccgatacgcgctggatc |
| | | cctca |
| 435 | Aichivirus A | cacccatacacccccacccccttttctgtaactcaagtatgtgtgctcgtaatcttgactcccacgga |
| | FSS693 | atggatcgatccgctggagaacaaactgctagatccacatcctccctccccttgggaggacctcgg |
| | | tcctcccacatcctccctccagcctgacgtatcacaggctgtgtgaagcccccgcgaaagctgctc |
| | | acgtggcaattgtgggtccccccttcatcaagacaccaggtctttcctccttaaggctagccccgat |
| | | gtgtgaattcacattgggcaactagtggtgtcactgtgcgctcccaatctcggccgcggagtgctgt |
| | | tccccaagccaaacccctggcccttcactatgtgcctggcaagcatatctgagaaggtgttccgct |
| | | gtggctgccagcctggtaacaggtgccccagtgtgcgtaaccttcttccgtctccggacggtagtg |
| | | attggttaagatttggtgtaaggttcatgtgccaacgccctgtgcgggatgaaacctctactgcccta |
| | | ggaatgccaggcaggtaccccaccttcgggtgggatctgagcctgggctaattgtctacgggtag |
| | | tttcatttccaattcttttatgctggagtc |
| 436 | Aichivirus | tactccattcagcttcttcggaacctgttcggaggaattaaacgggcacccatactcccccccaccc |
| | KVGH | cccttttgtaactaagtatgtgtgctcgtgaccttgactcccacggaacggaccgatccgttggtgaa |
| | | caaacagctaggtccacatcctcctttcccctgggagggtccccgccctcccacatccccccccca |
| | | gcctgacgtgtcacaggctgtgtgaagcccccgcgaaagctgctcacgtggcaattgtgggtccc |
| | | cccttcatcaagacaccaggtctttcctccttaaggctagccccggcgtgtgaactcacgttgggca |
| | | actagtggtgtcactgtgcgctcccaatctcggccgcggagtgctgttccccaagccaaacccctg |
| | | gcccttcactatgtgcctggcaagcacacctgagaaggtgttccgctgtggctgccagcctggtaa |
| | | caggtgccccagtgtgcgtaaccttcttccgtcttcggacggtggtgattggttaagatttggtgtaa |
| | | ggttcatgtgccaacgccctgtgcgggatgaaacctctactgccctaggaatgccaggcaggtac |
| | | cccaccttcgggtgggatctgagcctgggctaattgtctacgggtggtttcatttccaattctttcatgt |
| | | cggagtc |
| 437 | Aichivirus DV | tactccattcagcttcttcggaacctgttcggaggaattaaacgggcacccatacacccccaccccc |
| | | ttttctgcaacttaagtatgtgtgctcgtaatcttgactcccacggaacggatcgatccgctggagaa |
| | | caaactgctagatccacatcctcccttcccctgggaggaccccggtcctcccacatcctcccccca |
| | | gcctgacgtaacacaggctgtgtgaagtccccgcgaaagctgctcacgtggcaattgtgggtccc |
| | | cccttcaccaagacaccaggtctttcctccttaaggctagccccgatgtgtgaattcacattgggca |
| | | actagtggtgtcactgtgcgctcccaatctcggccgcggagtgctgttccccaagccaaacccctg |
| | | gcccttcactatgtgcctggcaagcatatctgagaaggtgttccgctgtggctgccagcctggtaac |
| | | aggtgccccagtgtgcgtaaccttcttccgtctccggacggtagtgattggttaagatttggtgtaag |
| | | gttcatgtgccaacgccctgtgcgggatgaaacctctactgccctaggaatgccaggcaggtacc |
| | | ccaccttcgggtgggatctgagcctgggctaattgtctacgggtagtttcatttccaattcttttatgtc |
| | | ggagtc |
| 438 | Murine | gtaacttcaagtgtgtgtgctcgtaatcttgactcctgccggaatgccgcccggttcagtgaacaaa |
| | Kobuvirus 1 | cagctaggcaagtccctcccttcccctgtggtcggttctcaccggccaccatccctcccccagcct |
| | | gacgtgttacaggctgtgcaaagcccccgcgaaagctgctcacgtggcaattgtgggtcccccctt |
| | | tgtcaagacaccgagtctttctcccttaaggctagcccggtcccacgaacgtggaactggcaacta |
| | | gtggtgtcactacacgcctccgacctcggacgcggagtgctgttccccaagctgtaaccctgacc |
| | | caagactgtgctgcctggcaagcaccgtctgggaagatgttccgctgtggctgccaaacctggta |
| | | acaggtgccccagtgtgtgtagtcttcctccagtctccggactggcagtcttgtgtaaagatgcagt |
| | | gtaaggttcaagtgccaaatccctggaaggagtgaccctctactgccctaggaatgctgtgcaggt |
| | | acccccaacttcggttggggatctgagcacaggctaattgtctacgggtagtttcatttcccatcctct |
| | | cttttttggcatc |
| 439 | Porcine | tttgaaaagggggtgggggggcctcggccccctcaccctcttttccggtggccacccgcccggg |
| | Kobuvirus K-30 | ccaccgttactccactccactccttcgggactggtttggaggaacataacagggcttcccatccctg |
| | | tttacccttactccactcacccctccccttgaccaaccctatccacaccccactgactgactcctttgg |
| | | atcttgacctcggaatgcctacttgacctcccacttgcctctcccttttcggattgccggtggtgcctg |
| | | gcggaaaaagcacaagtgtgttgttggctaccaaactcctacccgacaaaggtgcgtgtccgcgt |
| | | gctgagtaatgggataggagatgccaataacaggctcgcccatgagtagagcatggactgcggt |
| | | gcatgtgacttcggtcaccaggggcatagcattgctcacccctgaatcaagtcatcgagatttctct |
| | | gacctctgaagtgcactgtggttgcgtggctgggaatccacgcttgaccatgtactgcttgatagag |
| | | tcgcggctggccgactcatgggttaaagtcagttgacaagacac |
| 440 | Porcine | ccaccgttacttcactccactccctcgggactggtttggaggagcataacagggcttcccatccctg |
| | Kobuvirus XX | ttcaccctcaataccacccaccctttccctcaaccatccctatccacaccccactgactgattcccttg |
| | | gattttgacctcagaacgcctacttgacctcccacttgcctttcccttctcggattgccggtggtgcct |
| | | ggcggaaaaagcacaagtgtgttgcaggctaccaaactcctacccgacaaaggtacgtgtccgc |
| | | gtgctgagtaatgggataggagatgcctacaacaggctcgcccatgagtagagcatggactgcg |
| | | gtgcatgtgacttcggtcaccacgggcatagcattgctcacccgtgaatcaagtcattgagattcct |
| | | ctgacctctgaagtgcactgtggttgcgtggctgggaatccacgcttgaccatgtactgcttgatag |
| | | agtcgcggctggccgactcatgggttaaagtcagttgataagacac |
| 441 | Caprine | gggggtggggggggcctcggccccctcaccctcttttccggtggccacgcccgggccaccgat |
| | Kobuvirus | acttcccttcactccttcgggactgttggggaggaacacaacagggctcccctgttttcccattcctt |
| | 12Q108 | cccccttttcccaaccccaaccgccgtatctggtggcggcaagacacacgggtctttccctctaaa |
| | | gcacaattgtgtgtgtgtcccaggtcctcctgcgtacggtgcgggagtgctcccacccaactgttgt |
| | | aagcctgtccaacgcgtcgtcctggcaagactatgacgtcgcatgttccgctgcggatgccgacc |
| | | gggtaaccggttccccagtgtgtgtagtgcgatcttccaggtcctcctggttggcgttgtccagaaa |
| | | ctgcttcaggtaagtggggtgtgcccaatccctacaaaggttgattctttcaccaccttaggaatgct |
| | | ccggaggtaccccagcaacagctgggatctgaccggaggctaattgtctacgggtggtgtttcctt |
| | | tttcttttcacacaactctactgctgacaactcactgactatccacttgctctcttgtgcctttctgctctg |
| | | gttcaagttccttgattgtttttgactgcttttcactgcttttcttctcacaatccttgctcagttcaaagtc |
| 442 | Rabbit Kobuvirus | gggctataaatatgggcattcctcttcccccttccccttttgaagatgagtgcgcatattcttgactccg |
| | | cctggattggccgcccaaggcgtgaacaagcagctaggccaccatgacactgcggtggtgtccg |
| | | aacccgcgggtgccttcacgggcacctgtggtatgtaggactcccaccgtggtcttccctttccccc |
| | | tcaatctttccccctggttcgactaacgggaccagtgctggaacctgtccggtgaacggtatagca |
| | | ggcccccccggcagaaacacccggtgcttaccccttaaggctagcccccttccatgaatttggttg |
| | | gggcaactagtgggtgtacagttggcgtgaaccctccggtctaggagtgctcttgcccaatcctct |
| | | gtgtgtgccttgcagtagggactggcaatccttcgcgtaggtgatccgctgtgccatgccatcctgg |
| | | cgacaggaggcccagtgtgcgcaacctacgtcccttctgggtgctgcattgcattacctttggagta |
| | | agcttggtgtgccgaaaccccagggtttacgtaccactcgtggtgtgaggaatgtgccgcaggtac |
| | | cccatccttgaggtgggatctgagcggtagctaattgtctagcaccactttcttccttttttctttgctgg |
| | | tcacg |
| 443 | Aalivirus | ttgaaagggggtgctcagggtagctccctgagctcttccctccaccctctttcaacgtctggcccac |
| | | gatacgggccacctttcaatcttaactaactatccctttaatctatttggattttctggtttagaataatttg |
| | | gaacacataattggattatcttttaggattgtggataggatttgttcgggatatcactcccttcctgtgct |
| | | aacacatattctaattccctcctttgtctattatctcttggaggtggtgctgaaatattgcaagccacttg |
| | | agtgtatagatgaagtaggctcaagatgaatgttgtgttactcaaggcaagtgtagctatcactaaga |
| | | tattggtaacgtgaaacggattaccggtagtagcgtgatcttccgtcttagtgctctagtgactagag |
| | | gacaacgacatggcatcacatatcttaaccctccagttttggcatccgggacagaatgggctggat |
| | | atccgctttctttctggggtatgtgatgggtggtattggggtaaccaccttgaccatgacgctcgata |
| | | agagtgaccgcctgatcattgaaacctctagtataaaattcaggctgaaatc |
| 444 | Grusopivirus A | tgcctgagtaggattgtgaatttaggtatgagagggttagccaacccattctgaaccataatagatac |
| | | gtcaatctgaatccatctaaatctatctcttaggcagtggtgctgaaatattgcaagctactagggata |
| | | gacgtgatctgattcaagaacctatctaatgtggtgatgagaaggctaggtttatccatagtaatccct |
| | | tgttctgaacaggcaatgcacatgctctagtaggatctcgggctctgcgattggctctaaaccgacc |
| | | aatccaggtagaggcactaagtgtaggacttgccaaaatgtattacatgctggtaccgactcactag |
| | | tctggaaactccacactgaaagtgactggggggggccccatcacatttgtgctactgcttgataga |
| | | gttgcggctggtcaacttggattggtataaccagttgaa |
| 445 | Grusopivirus B | gccatccgtaggttctggtaaggttccatcaactgttggggcgctagttgctatgaccgcattcacg |
| | | gacggatgatttatagtatcacccaatccgggcacaacttctttagccacttcttccacattactaagg |
| | | gctctcttgccgagtttcaacgtctagtccacgacacggaccttcctacttttctatcttcttattttctct |
| | | actaaattggtatctggtactgaagatatgcggattgtgattttgtgcctgtctaaactaaccctattcta |
| | | gggttaggtgggtaccatatactaatggtgaacaggattacctatgtatccattagtccctatggatct |
| | | ggcgacccacaaactcatgttcatagagaggctaagctgagtgctcgccgaataagcattgcttca |
| | | ggtgccgactattgtctggaaaccactcagtgatagctataggggggggccccgtagcatctgcct |
| | | tactgcctgatagggtggcggctggtccatgaacatgcagtaaccagttgacttgac |
| 446 | Yancheng | ccttagggtctggaatgcgtcctttctgggcacttccacaatcctaaggtaattttcaacgccagcga |
| | osbecks grenadier | tggagcgatatccaaagaacctttatgttttagttcgtcttgtatgtttataaaatataaaattgggatta |
| | anchovy | gcaacccacaaaaacatttttgttattctcaccacatgagagggtggttaaacctcttcgtaacctatc |
| | picornavirus | ttgcttgattggctacttgggctagatttaggacaacctctttagcaagcctaaattactcctcgtactt |
| | | gcacctggtaacaggcgtacctggaggttacggtggcgctaacttggacttctcgttaattcgtgca |
| | | agtttaaatgatgcctattttgaatacaagaaagtatgatagtaacttagggcgtgaagttccgcttaa |
| | | cataaggcagtataagtactaagataaggtgtaagacctaccttaataactgttgtcttttctcatggtc |
| | | tttccgtgggagcccttgctaggggtaaagttaagtattctcaaataatttttcattcaaactctttctctc |
| | | tgtttt |
| 447 | Turkey Gallivirus | ccactcgcacttcctggatagtgcgttagatatgcccgacatatcgcttccaggaccaaagccccc |
| | M176 | cttttctctttcccaaccagcttcgccactcaagctgtaattccatgtccggtctttccggccttagtatc |
| | | atggaaatgtggtcgtgctcaaatgaaattgagttgacattgatcaatgaaagttgcactgaactttg |
| | | ctaaactggctagcgccacctggtgtgtgccgttggtctcctcacatggtaacatgtgccaacggg |
| | | cccgaaaggctagtgggcaattaccgctccaagggaggggtacccaccccgacctgaacagcg |
| | | gtaatgaagctcacctcccaggctctgaccccgagaagtttagttatttagtaggtgtaattagtactt |
| | | gtgattggtcaatttgatagtagtttgaaacgttatggatgaatgagtagaccccctgaaggtacccc |
| | | attacatgggatctgatcagggccacattctgcgtgtctccccgcacttgtggttaaaaccatgaaa |
| | | gttcatcccaaacaatcttttcctcttctttttcttttagtggtgacaacctactggattggtgattaccaat |
| | | ctgtactagtgttgtattaagacttgttgtgtggagaaaatggactctttcaagaagatttttg |
| 448 | Falcovirus A1 | taaaaggggacgcggtgtggcagctttggctgtcatgccgtgttctccttttaccccaaggactagc |
| | | cttgggggttttccaaattcctttccctgtaggctttacttctctttatctatcttttctgtaactaagttttgc |
| | | ctattctaaaaatattttagaatgtgtttggatgtaactaagtttgtgcctgccctaaaaatattttagggc |
| | | ttgtttggataacctcgtcccttgtgttcagtgccgcacaatttgctaggcactgttcacttcctttgtttg |
| | | tccattatgtatgctaaggtatgaattccatcatatgcttagcctctacatgcataatcttattccctccct |
| | | ggtgcaaactacgcccccaacatatgtgaatcttttaagcatattcctgaccccacacatatatatgtg |
| | | ttctcgtgaattcccccaccgtgaggtggtcacttggacgtggtgtgtgtcacacagcatatatatga |
| | | tgcaggatgttgtttttaagataagcatatgtccttagtgctttgcatcatttcctccacaccccgtgaat |
| | | gcggctaatcttaaccctgttgggtccgtgggtaaaccaacccattaaccacaggacggaaccga |
| | | ctactttcgggagtgtgtgtttctttttcttcttttgtcact |
| 449 | Tremovirus B | ttcaaatggcccctgggttgatacccagtggtcatttggacactttggtaaggaggtgtaattatcctt |
| | | cccatgtggaacctagtgcttaggtttactttatatgttctttgtttgtcctttgtactttctatcgggcaat |
| | | cttgttgttcaatacaatatgtatttgaactgcctaagataaattcagttttcaaccaacccctctcttgg |
| | | ggttgtgtctttctttctttcttatatcctcttaagctgacttacttgctaatccgactcctcgtcaacggg |
| | | agggtaaagcagtatcactagggtattgtgatgtaggagaaaaagtaagtagagatagtgcatgta |
| | | acgaaagtgacttggtactttaaactctcttaatcccaaagtgtggtattggtcatgttggagtaggct |
| | | acgggtgaaactccttcacatttagtaatgtgttcacacgctaacgctacggtagatgacagactag |
| | | gtcttattctcaacgtagggggacgggtgtatgttcatgattagccacatattaaggttttgaggggct |
| | | gagtcatataagtatgtgcattaatttctggtactggtccctggggactggcccttttctaggttgatttt |
| | | agtttccccaatttttaaaaactaatgagatttacgac |
| 450 | Didelphis aurita | tctttggtctggggaactaaaataccagacccgcgtttgcctagcgatataggctttaattgttgtttgt |
| | HAV | cattgtgcgtttgatatgtgttttaatgtaaataataattctagcaggttctagttcttgatcatgtcctcttt |
| | | aaggcactcatttcaacttgctatctttcttttcttccttggttctccctacaccaaatgcactggccgct |
| | | gcgcccggcggggtcaaccacatgattagcatgtggctgtaggtgttgaaggctgggacatgaac |
| | | atcaatggaatagtgcgcatgcttactggggtccattgaagtagtgggatctttctattggggtaggc |
| | | tacgggtgaaaccccttaggttaatactcatattgagagataccttggataggttaactgtgctggat |
| | | atggttgagtttaacgacaaaaagccatcaacagctgtggacagaacctcatccttagattgctcac |
| | | tatggatatgtgctctgggcgtgtttcttgcatgatggccattggtcaattcatgcctgggccaatgta |
| | | ggattagccttaaattactttttaaaagtagcctcatttagctggactaatggtggggcgtatgatcctg |
| | | catttggcctctggggtaatcaggggcatttaggtttccacataatagcaaat |
| 451 | Hepatovirus G1 | gcaaggggtggttttaaccttgcacgcgtttaccgtgcgttaacggttttccatgtttgtatgtcttgttt |
| | | gtattatgtgttttgtaaatattaattcctgcaggttcagggttctttaatcatgttgggctgtacccacac |
| | | tcaacttttggccataagtgagtttcttaacgaaccttttaacacaggatgttattagggcccaatatttt |
| | | ccctgaggccttctttggcctctattttttccccttttctatctccttgtattccgggctcacgtgatgcca |
| | | atggactgacccatgcgcccgtgggggttaactactggagtagccagtagctgtaggtgctaaaa |
| | | gtcacgtacgtgtaagactggacgagacctctcagctataactgaaagtagtaagtatgtctgaact |
| | | tcttgaaggggtaggctacgggtgaaaccccttaggttaatactcatattgagagatacctctgatag |
| | | gtgaaggtttccggtagaggtgagtttaacgacaaagcctctcaacggatgtgggcccacctcatc |
| | | agcaagatgctttcatacccaataccgtaggggctgggttgttgagttcagtcccaagcgtccctcc |
| | | cgcaaggttgtaggggtactcaggggcatttaggtttccacaattaaacaaataca |
| 452 | Hepatovirus D | ctttggatgcccatagtgcgggggtataaataccgcactccctttagctgttccgagggtatcggaa |
| | | cctatatgtttgttttctgtctgtctgtcagctttatgtgtgctcgtcccctttagggcactcatttcagctt |
| | | gctttcattcttttcttccccggttctcaccttaccggaggcactggccgttgcgcccggcggggtca |
| | | acctagtgattagcactaggctgtaggtgtctaaagtggtgacattaagacttggtaactgatttcag |
| | | cactgttaactgatgttggggatgacttgattgatcttctggaaggggtaggctacgggtgaaaccc |
| | | cttatcttaataccactatgtagagatagattcagtaggttaagggcagtggataaggttgagttcattt |
| | | tggacaataaaccttcaacactggtggacccaatctcactgaccagatgctttcttgactgatccttc |
| | | agaggggtgattcttctgaataggttgccttgacactgatgcctgagacccattgggtcgggcctta |
| | | aatcatggaactccactggactttcatggcctagcttctgccttagacagactctggggccccacga |
| | | ccctctgggcccttcggggtactcaggggcatttaggtttttccacaattaaaagagtta |
| 453 | Hepatovirus H2 | gtcatgtttctctttaagaacactcaattttggccataagtgagactcttgtcgaacctttcatgtcagg |
| | | accatgttagggccattatccttttccctggggcattcttcttgcccctgtttcatctttctatcatctttctt |
| | | ccgggctctcacaatgccaatggagcgaccgatgcgcacgtcggggttaacccatggattagcc |
| | | atgggctgtagctgctaaaagttgtgactcctgaagcatactatcaatggtagtagatgtaactgaaa |
| | | cactgaagcttctctgatcttgaaagaagggtaggctacgggtgaaacccttcaggttaatactcat |
| | | attgagagatacctttggtaggttaacgttggcggataatgttgagtttaacgacaataaacattcaac |
| | | gcctgtgggcgaacctcaccaatttcatgctttgaagtgaatgtgcgtagggtctctatcggagatg |
| | | ctatgtggatggtgccctccctggaaacaggttgtaggggtactcaggtgcacttaggtttccacatt |
| | | ttaaagatttttc |
| 454 | Hepatovirus 1 | ggctgcctgtgtctcaggggtaagtactggggccgcgttgaccgtgcggtacggttatgcttttaga |
| | | ttaggatgtccgtctgtccggcactctcttttgcttaaaatggccttaaatccatgggaggcgtaacca |
| | | tgggccctttgttacctagacatgattgcattgggggccgtccttggggcttaggccccagccatttc |
| | | tcttgactcgtctaagagtttacttcatccttttctttactttattttccaggctctcagcatgccgacggct |
| | | ctgaccactgcgcccggtggggttaactgcatgattagcatgcagctgtaggagttaaaagtgctg |
| | | acaggccaattctgacgtaagtccactctatattaacttgatcaagtaaggttgattgatctttgtgaga |
| | | gggtaggctacgggtgaaaccctctaggttaatactcatattgagagatacctccagaaggtgaag |
| | | gttggcggatattggtgagttcttttaggacaaaaacctttcaacgcctgtgggcccacctcactggc |
| | | acaatgctttcatccccaattgtgatgggtagtttggactgaaatcaggagtaacctgccctacgagt |
| | | ttaggggtagttcaggggtatttaggcttccacatttgatagagtttatgagagtgagcc |
| 455 | Hepatovirus C | ttcaaaagccccagcggggtttcattaccccgctgtggcttttggacttccctaggatggggaagta |
| | | aattaccatcctcgcgtttgccgtgcgttaacggctacttttcttctagctgtagaagtaaaattcagca |
| | | tgttttatgtttgtttgtcttgtttgttatatacatttttacactcctacaaatgcacatgaagaacagtttgt |
| | | agagattaacaaacgcttagctgaacctaggtggtgaatctagtagtaagataagtagaggaagct |
| | | ataccttaagttggttgggccctcgtgtttgctctataaacaaaaccaagtgagtagagtggatgaac |
| | | agtactaaatccctgagtacagggaacctcacaggtgtgatacacttatgtctatgtgacctggttgg |
| | | aggttgggcgtgccctatgatactggagtgggagatcttttggggaacccacgttttcacactgcct |
| | | gatagggtcttgccgagagactcacttgtttcggctgtacttgtaac |
| 456 | Fipivirus A | tgcgggtaaactcccgcatgtgtgaatgaggcgatgtcccaggaactaactgccgatcctggtttta |
| | | actacgatccgtatttgttactaatgcgatatccccccattgtttgcctccatgttgttttcaacgcttttg |
| | | gccttgagtgttatcaagtgttttagcgacatagtgggaagctacggctgcgtccccatttttgagtg |
| | | gcgacccagttttagtggccactctgtccctgaactgcgctataatgtgaatttatgttcacaaaaac |
| | | ggactgatgtaactgttaatgactaaggaatagtacctcactgaagtatcaagaccccgttcgagcg |
| | | gtgtacatatatggatggaaaccttgtctgagtcatctcgaatactaatcaatgagggatgtcgagta |
| | | agcatatcatgaaccacatagaatagtggggtttcggggttagaggctctctgcagcaatgtatctct |
| | | aacaccatggccgaaatgagagatagagaccacgatgtttgtgtgtaagtaatgatgtgtggaaag |
| | | aaaattctgaatgttggtatgatatcagtctaaggggagtggctcacctaagagctacccaaacattt |
| | | cacagcagacaacataacgtactgagagtagttggaaggttccagaaatcagt |
| 457 | Fipivirus C | cgcggttaaacccgcgcaaccttctttcagccgcgtctgagtagcgcggttagtcctgatacacagt |
| | | ttcctgttgggtactgtgtcttcgggtgaatgctcttgtgtgaatgttttaggctgtttaagggaagcgtt |
| | | tccccgtgcgctgtgagggtttctcacgctctttcggggtgcagtctcttctgttgttcattaagatgta |
| | | tggatgcactgttgtgaaggatttgtgaactggggatcgacaccccgtgaggggtgccccagtgtc |
| | | cataggagtttgctggagaggtgtgttgctgtagtgactatccgtgacctggcattctaaggtgttga |
| | | ccccaacctgtgagggtctggatcgcagtgttgaagtgctttggagggttcaatggggtttctgtagt |
| | | ggatattatgtgcttgacgactactggtacgagtgtattgggggtctacatgtgtga |
| 458 | Fipivirus E | ctcttccgatcttgggggttcgcccccatgtctcatttcaactagccgtgtgtctagttaacgcaccgc |
| | | ctcaccctggtcgttatcgggtcggttcttgcgaccgttagatcgtgagcgtttctgaggatcagttc |
| | | gtataagttctccggtgtggcgaccgtaaaatcgtcacgtcccatgcaatagatgacgttaaactcg |
| | | tttgccagttacataaaggaatgttgttacttttaaattgtctgttacatttaacatcttgccagtatgatg |
| | | ctactgtacactacgggtgtaggaaccttgtagtgtgacgtatcactcatatgtggatgggtgctcca |
| | | gacctttatggaagctctcagttagtagtgatccttgacttcattgagccctggtaacagtggaagtc |
| | | aagatgtatatgttgctcaacacacttcggtgctacgaagctgtttgtggaagtactggcgaggttca |
| | | ttctgaatcatatgtttgtcacatagtcagggagtgccgtcgcttacgacggaccctttttctttataatt |
| | | acaaatctgtgtctcaagtgttgttggctggttttcttcttctgttttcattgttcatatatatacgtcagagt |
| | | gaaagactcggtatatacaaaactgatccaga |
| 459 | Aquamavirus | ttcaaaggtggcgggagagttggcctcacgctgtttagcgtgagagctggctctcctgccccttcc |
| | | cctgagccggggatcttggctcattcccctcttttctatcctccctcattggactttacggatgacccg |
| | | gcataaacttgacaaccgatgttggatttcccttgtggctgtgatggaggacataccctcgggtgta |
| | | gttgtgtgcgtgtcgctctgcgactcgagcttcaaagtggtgctgaaatattgcaagcgtcgttgctc |
| | | gattaacggagtggtacaatcctatgaacccaagtgcattcatgcgaaagccccggaggggtgag |
| | | tagcatggactcgaatcagaagagctggagctcgcttggtacggcacgtagcattgctttgcctaa |
| | | agaccaagggggtatggctataggtgggggcctatagcttgtccagtgctggttgacagactcgtg |
| | | ctacgcgtctggttcgagtataagtagctgcaactcact |
| 460 | Avisivirus A | ttcactcgctttccccccctctctataggggcggtcttttaattcttattaatttcctactttactatcaaatt |
| | | tcttctaagtagggactgaggtcacttagccctccctctcctgggctttccagggttatagaggttcta |
| | | aagctaagccatgtgtcttgagctacacttagtacaaaggtttagtaatgattgtacatgccagtaac |
| | | cttctagtgcccatggattaaagagtggtaacactctccatggggcccgaaaggctagtgggcata |
| | | gttggcatcaaggaaggggtccccaccccaacctgaattgctggctagaagctcaccttagaaga |
| | | agtgctgggtgacaacgtgtccaatcgtgaacgactgatggaaacgtgtggagatggatatgtgg |
| | | gggttcactgagtagatgccctgaaggagaaatctgatcaggggcccgtgactatacgctaggta |
| | | aaccgggtataaaaaccatgaaaggtggcccaaaatctcttccttttattttatttctatgttggtgaca |
| | | gtcaag |
| 461 | Avisivirus B | caccccctactgccctaacccccaaagttagttatagggtggctccctacccttactccacggggta |
| | | agccctaacccggttgaatctcaagatcagccttagcgaggactattagtaccgctcaaaccctttg |
| | | cctgtagtgcccaggggtcacagaggggtgaccctctccctggggcccaaaaggctaggtggca |
| | | agacagggtccaagtgaggggctactctaagtagccccaagctgaacatcctgtctgaagccacc |
| | | cttgcagggccaggtttgattggggaaactagacaccagctttgtcctgggattggggggatatcg |
| | | agttagtccaggaggtgcgagtagatgcccccgaaggtaccccaggcacatctgggatctgatcg |
| | | ggggcccgtgactatacaataggtaaaccgggttaaaaaacatgaaagcgcctctctctttcctact |
| | | tcttttattgactggtgacaaaaatagcagt |
| 462 | Crohivirus A | gttgaagtccatttcttgcttgcccccgatgaatcctgttaaggcctcacggccctaagggtgaaact |
| | | cggttatcccctcctgtacttcgagaagattagtacaacactatgaaatctacatcttgtgatccggga |
| | | taaccccaatcccagaaacctgtgatgggcgtcaccacccctcttatggtaacataagggtgtcgc |
| | | cgcgttggcacaggaccctttgggctggatgtttttagtaatggtgtcgaaggtcctattgagctaca |
| | | ggagtttcctccgccctggtgaatgcggctaatcttatccctgagcctaaggttgcgatccagcaac |
| | | ttgatggtcgtaatgcgtaagttgggggcggaaccgactactttccagaaggcgtgtttctttgttttg |
| | | tctgttactatggtgcatgatatagatattgaatatttgatctttttgagctgtttcttatcttattgctacatc |
| | | ctttcaggtgttggatttacattttggttaataag |
| 463 | Kunsagivirus B | gattttctggttatcccttttggacttggtaggggcccacgtgcccacccacctctgtgtgtgttgattt |
| | | ctaatcgatgcctggcagtggcggccacctctccttactggtaaacctccggtgagtgaagttgtca |
| | | agctacaggtaccgtgcaggatgaaatgcgcacatgtgaacaaactaggagtcatacaccgggtc |
| | | aaactctggaaacggagtccgggactctgaccttggttgggtgagctcgaggcatcacattgatgg |
| | | acgcgattcgctatccttccctagtaggaccttgtggtgtacccctggttgggaatccagggctggt |
| | | cgggtgcagggtgacagcctgttctccacctcaaccattgtaggagaaatcaacccct |
| 464 | Limnipivirus A | ttctttggatatccatttaacgtgtaccctatacgataattggggtggattctggatgcctagttccagt |
| | | gattggttaagaactcgtttactacgtatagtatgattagcaaagtgctcgattgatcacgtaatgatct |
| | | atgtggttaaaaacccagtagtatggtatatactcagtagtgtacactgtgagtacaactcttggcgta |
| | | gagagaacaattcacccgaatccgtggcgtatccatggaaataagtttacctaattgtatgttacaag |
| | | gcatatgagacatttatgagatatggtttattttgactaaacgagtgtagaggtggtggagtctatcca |
| | | acttcaagccatgcaattgttgtgttgattgatatcattgaccatttttgtggattgtgtacacatacaatt |
| | | tgaaaattaaccccctcaagaataagacatgggaccattcgtggtagataccgtgctcggatgcttg |
| | | agattagatgggttagactagttttggaatgagattgccgagaaagtcccgctagacatgttttacaa |
| | | gtcgtggtattccgctagactttttcgcagacacatggaagggtccatgtgttgtgcaattgcagggt |
| | | gacagcccaactgcagagttttccttactagaataaaaatctgttgtcaatttt |
| 465 | Limnipivirus C | gtttctgagcactggtaagagcttagacaaacgtttttaaaatttattttctctgcaacttttgtttgtgttt |
| | | atttttatttgttaattttgcgcctaagcatttgttgcgaagtatttgattcattagtaatattacttattgttta |
| | | tttagatggtattcaaagtggtgggagtatcgaacccaagcgtcgtatgctatctccttgaacaattttt |
| | | aatcattgcgaagtgatcattgaaaaggataggtgtttaagaactcaaagagtgttaataatgttggg |
| | | tgacaggtgtccccatagaatttattaacatgatttggactggttatctagtaagaagaaccatcgaa |
| | | cgcacgagcgagcattgcttgcggggcagttaccctgcgtcgatgtaagtgtgtaccggggggtg |
| | | cacatgttgattctttatggcctgatagggtgcgtcattcgcgcctagataattagtataatgcgaatg |
| | | gaataaatttac |
| 466 | Orivirus | ggtcccaggccaatattcttcgtaaggcttggttccaattttccaccactcgtgtttgggttctggccta |
| | | tggtacccagaggggcggtttgggggaattaactccccctcccctgtggtcctataccaccccaca |
| | | cctctgtgggctttctttactatcttcttgttttccgacttttaaacactaggcaggcgcgcctagtcata |
| | | caccgcccggctggtctttccagctcttgtgggcggtgcgcgctggtccatcgtgcccagcgacat |
| | | agcaccttgtggacacctccgaacgccctcccctgtatggggtggtgcccaggggtttcagtgtgg |
| | | tgacacactccctggggcccgaaaggctagtgtgcaacaggtgaggtacagccagctgcccccg |
| | | tggctggagggaccaagcttgtgaagcacacctcaccttcttgggggtgggctagtaagtggtga |
| | | aagcatagtgtccgtgtcgctggccaacactttgggtcaagtccagccactcagtgagtagatgcc |
| | | caggaggtacccctagtggatctgacttggggcctgttacttaatgcaggttaaaaactatgaaagc |
| | | tgagtagtgtagcccggctggtggcttctcttccttattcattctattttatggtgacaaacgcaactga |
| | | agcc |
| 467 | HAV FH1 | cttgatacctcaccgccgtttgcctaggctataggctaaatttccctttccctgtcctttccctatttcctt |
| | | ttgttttgtttgtaaatattaattcctgcaggttcagggttctttaatctgtttctctataagaacactcaatt |
| | | ttcacgctttctgtctcctttcttccagggctctccccttgccctaggctctggccgttgcgcccggcg |
| | | gggtcaactccatgattagcatggagctgtaggagtctaaattggggacgcagatgtttgggacgt |
| | | cgccttgcagtgttaacttggctttcatgaacctctttgatcttccacaaggggtaggctacgggtga |
| | | aacctcttaggctaatacttctatgaagagatgccttggatagggtaacagcggcggatattggtga |
| | | gttgttaagacaaaaaccattcaacgccgaaggactggctctcatccagtggatgcattgagggaa |
| | | ttgattgtcagggctgtctctaggtttaatctcagacctctctgtgcttagggcaaacactatttggcct |
| | | taaatgggatcctgtgagagggggtccctccattgacagctggactgttctttggggccttatgtggt |
| | | gtttgcctctgaggtactcaggggcatttaggtttttcctcattcttaaacaata |
| 468 | HAV HM175 | cgccgtttgcctaggctataggctaaattttccctttcccttttccctttcctattccctttgttttgcttgta |
| | | aatattgatttgtaaatattgattcctgcaggttcagggttcttaaatctgtttctctataagaacactcatt |
| | | tcacgctttctgtcttctttcttccagggctctccccttgccctaggctctggccgttgcgcccggcgg |
| | | ggtcaactccatgattagcatggagctgtaggagtctaaattggggacacagatgtttggaacgtca |
| | | ccttgcagtgttaacttggctttcatgaatctctttgatcttccacaaggggtaggctacgggtgaaac |
| | | ctcttaggctaatacttctatgaagagatgccttggatagggtaacagcggcggatattggtgagttg |
| | | ttaagacaaaaaccattcaacgccggaggactgactctcatccagtggatgcattgagtggattga |
| | | ctgtcggggctgtctttaggcttaattccagacctctctgtgcttggggcaaacatcatttggccttaa |
| | | atgggattctgtgagaggggatccctccattgccagctggactgttctttggggccttatgtggtgttt |
| | | gccgctgaggtactcaggggcatttaggtttttcctcattcttaaataata |
| 469 | Parechovirus F | ggtcggggagatgtgttcatgatcggttaacaccatcatggatcatctctccccgacctctttttgacc |
| | | cagctatgggttaaatagtacttttcttttctcttttgctttcttttgtgttttgtttgttttgcaacatataaca |
| | | agcattttatcagtattagtgtctgcaactgtataacaagcaaggtggagcaatcatgcgagtatatct |
| | | caattgaattgtgacacacaagtgtgcactatgtggaataaatgccattttggccaaacctggttagc |
| | | cagaccagtagtaggacaatttggcacccttagtgggcgcgacctagatgctagggatgagcaaa |
| | | cctatttcccctgagtacaggggctctccttcacctctacattttggacctctttttgagtatcctcgata |
| | | gaaggtgaagtgacggtgtaccggatggttaattgatctcattgctgggtgacagcccgctaggac |
| | | caggcagcatctttgtatggacctgtacatgtaac |
| 470 | Parechovirus D | acatggggcaggtgtgctgtgccaagagcaacactacggtggccgagccgatggttcgtcacca |
| | | cgtagtaggactccgtagtgcttggttacggcggacgtaagtcagttgagtgatgtctaagtggcaa |
| | | accatgagtacatggtaaccttgtgtggactcgcgggacggaatttcctatcccattgactccttgta |
| | | gcaaggtgggtatacccaaccacaatggcagcaccctgggtgggaacccaggggcctggatta |
| | | gtatccagtcacacagcctgatagggtggcggctcagccactgaccagcgtctctaaataattgtg |
| | | agctgttcatgcacc |
| 471 | Parechovirus C | cggtcatccccctttccccacagccggtgtgggttctaatcggctcctactaaacacctaagcatca |
| | | ctgcgcctctatctctcctatccacaggtctaagacgcttggaataagacatgtgggtgcaatagga |
| | | agattagctagtccaatctctccttccagctacgcttctcccttcgatgagcgtagggggggccccc |
| | | acctccctcatctctggatagggctcttgctacggggctttcccgtctggaccagcaggcccactg |
| | | gtgcgcttccattcaagtttagtgtgcattactgtctgaaatattgctttgctaggatctagtgtagcga |
| | | cctgcatattgccagcggacttccccacatggtaacatgtgcctctgggcccaaaaggcatgtcttt |
| | | gaccgtatgcagtacaaccccagtataggtcctttctatggcagtatggatctcagtgatgagtctat |
| | | acagaatatggaagtggttcggatatgtcagcccgaaggatgcccagaaggtacccgcagataa |
| | | ccttaagagactgtggatctgatctggggcccaccaccttcgggtgggtagaagctaaccatgcct |
| | | tgggttaaaaaacgtctaagggctgaccagacccgggggatccgggttttccctatcttgacctact |
| | | ctaatc |
| 472 | Ljungan Virus | ctcattgcccacacctggttggttcccaggttcatacaataaccatcaataaacttttaacatctaagat |
| | 87-012 | agtattatcccatactagactggacgaagccgcttggaataagtctagtcttatcttgtatgtgtcctg |
| | | cactgaacttgtttctgtctctggagtgctctacacttcagtaggggctgtacccgggcggtcccact |
| | | cttcacaggaatctgcacaggtggctttcacctctggacagtgcattccacacccgctccacggta |
| | | gaagatgatgtgtgtctttgcttgtgaaaagcttgtgaaaatcgtgtgtaggcgtagcggctacttga |
| | | gtgccagcggattacccctagtggtaacactagcctctgggcccaaaaggcatgtcatttgaccac |
| | | tcaggtacacaaccccagtgatgcacacgcttagtaatggcttagtaacaaacattgattgatcattt |
| | | gaaagctgttaggaggtttaggtatgacgggctgaaggatgccctgaaggtacccataggtaacct |
| | | taagcgactatggatctgatcaggggcccaccatgtaacacatgggtagaagtcttcggaccttgg |
| | | gttaaaaaacgtctaggcccgccccccacagggatgtggggtttcccttataaccccaatattgtat |
| | | a |
| 473 | Parechovirus A2 | gccgtcgggccttacaccccgacttgctgagtttctctaggagagtccctttcccagccagaggtg |
| | | gctggtcaaacaataccaaacgtaactaaacatctaagataacatagccctatgcctggtctccacc |
| | | agttgaaggcatcttgcaataaaatgggtggattaagacgcttaaagcatggagtcaattatcttttct |
| | | aactagtgatcttcactgggtggcagatggcgtgccataactctattagtgggataccacgctcgtg |
| | | gatcttatgcccacacagccatcctctagtaagtttgcaaggtgtctgatgaggcgtgggaacttatt |
| | | ggaaataattacttgctgcgaagcatcctactgccagcggatcaacacctggtaacaggtgcccct |
| | | ggggccaaaagccacggtttaacagaccctttaggattggttaaaacctgagtaattatggaagata |
| | | cttagtacctaccaacttggtaacagtgcaaacactagttgtaaggcccacgaaggatgcccagaa |
| | | ggtacccgcaggtaacaagagacactgtggatctgatctggggccacctacctctatcctggtgag |
| | | gtggttaaaaaacgtctagtgggccaaacccaggggggatccctggtttccttattttagtgtaaatg |
| | | tcatt |
| 474 | Parechovirus A3 | agagtccttttcccagccagaggtggctggttaaataatacctactgtaacaaaacatctaagatgta |
| | | acaaccacacacctggtctccactggccgaaggcaactagcaataaggcaggtgggttcagacg |
| | | cttaaagtgtgttgtacatattcttttctaacctgtgttttacacagggtggcagatggcgtgccataac |
| | | tctaacagtgagataccacgcttgtggaccttatgctcacacagccatcctctagtaagtttgtaagat |
| | | gtctgatgacgtgtgggaacctgttggagataacagtttgctgcaaagcatcccactgccagcgga |
| | | tctacatctggtaacagatgcctctggggccaaaagccaaggtttaacagaccctttgggattggtt |
| | | caaacctgaactgttatggaagacatttagtacctgctgatttggtagtaatgcaaacactagttgtaa |
| | | ggcccacgaaggatgcccagaaggtacccgtaggtaacaagtggcactatggatctgatctggg |
| | | gccagctacctctatcttggtgagttggttaaaaaacgtctagtgggccaaacccaggggggatcc |
| | | tggtttctttttaatttaagtaatcact |
| 475 | Parechovirus A8 | gggccttataccccgacttgctgagtttctctaggagagtccctttcccagccctgaggcggctgga |
| | | taataaaggcctcacatgtaacaaacatctaagacaaaataatttgccttgcacctggtccccactag |
| | | ttgaaggcatctagcaataagatgagtggaacaaggacgcttaaagtgcaatgatagttatcttttct |
| | | aacccactatttatagtggggtggtggatggcgcaccataattctaatagtgagataccacgcttgtg |
| | | gaccttatgctcacacagccatcctctagtaagtttgtgagacgtctggtgacgtgtgggaacttact |
| | | ggaaacaatgctttgccgtaaggctttcattagccagcggaccaccacctggtaacaggtgcctct |
| | | ggggccaaaagccaaggtttaatagaccctaatggaatggttcaaacctggagcattgtggaaagt |
| | | acttagtacctgctgatctggtagtaatgcaaacactagttgtacggcccacgaaggatgcccaga |
| | | aggtacccgtaggtaacaagtgacactatggatctgatctggggccaactacctctatcttggtgag |
| | | ttggttaaaaaacgtctagtgggccaaacccaggggggatccctggtttccttttattttactttgtcaa |
| | | t |
| 476 | Parechovirus A17 | ctctattagtgagataccacgcttgtggaccttatgctcacacagccatcctctagtaagtttgtaaga |
| | | cgtctggtgacgtgtgggaacttgtgggaatcaatattttgctttaaagcatccattagccagcggat |
| | | aaaacacctggtaacaggtgcctctggggccaaaagccaaggtttaacagaccctagtggattgg |
| | | tttcaaaacctgaaatattgtggaacacactcagtacctactgatctggtagtaatgcaagcactagtt |
| | | gtaaggcccacgaaggatgcccagaaggtacctgtagggaacaagagacactatagatctgatct |
| | | ggggctggctacctctattttggtgagtcagttaaaaaacgtctagtgggccaaacccagggggga |
| | | ccctggtttccatttattttacaaaggcact |
| 477 | Potamipivirus A | cacatggaaagcttttcgcttccatgtttacgcacacactctctttgacaccctgttgtatggtgttaaa |
| | | ctacaacatttgtctgtctataatcgtttattttgtttaccctatatgtacccaagtatttgattgcttgactc |
| | | acataagcatcggtaacccatactgttttatgagctactacctctgctgtctacatacattttatatgaat |
| | | ggtttgagctctgcctcaggatcaaacatggtaacatgttcctttggtcagttagaatcttattgtataat |
| | | ctaaggtgtctattagtacgtagaaagttgtaacacatatggggcctgatagccgctatctctgatgg |
| | | atgtaaggtaaccttctttaggtctgatacattctgcacaggatccaattttcggtgccctgtacgagt |
| | | gcactcttatgcacgaggacgagatatgctacaacccactgcaaatttaaacccaaactttaaca |
| 478 | Potamipivirus B | tttcaacgtcgtggctgacgttaaaaagccacaattccacttaccttttaccttttatgtttaatgtttgtta |
| | | gttttgtgatctttaacaaatagatctaaataatttgttggtaaccaatctcggatgtttcggctgcattgt |
| | | agtttatttatttcattttagttgtaggtggccactacgtcctggaatcatacatggtaacatgtacctcg |
| | | gcggttatccactattacgctaatctaagaatatttaaatgaaaatgtaagtgttacggctgactttgg |
| | | gcctgatagttaaatgctcgcactgacagatagtaccctcctttaggatcgattctgttacatgggatc |
| | | cattttggtgccccactgattcaacctctttgttgaaaaagagttagcatactacaaattttccaaacaa |
| | | aaaccctttttaatgactacaacttatgattttatgaattttactgctcttgaaaaagatattttgacattga |
| | | tcgctgtactgtttcagacattcattgcatccatttttgttggctactcctcacaaactcaaaacttttcca |
| | | cacgagaaaccttgtttattgaattttgcctttatattttggaacttgttgttggatttattgtttgcttaatta |
| | | ttgacctcacacctgttttaaacactacaat |
| 479 | Beihai Conger | gggacaaccccacagctggtacaaccattgtgggttggtctccaccctttttcaaccgtggcaactt |
| | Picornavirus | cggttaaagttgcaaatcccccctctccctattccacctcccttactttcactccccatatatggtccca |
| | | gattttattctacctctttatatttttatttagtacagtggtggtgaattactcccagcataaactttgctgg |
| | | atcagtgttcatcaagcatactaattactaatgtactgagctatactattatctggcatctcacctggat |
| | | aaccggtgtgaccatatttcctaggttgcctccctatgtattttgtagcacctgtgcatctgcacgttgg |
| | | ggcgacaaattgtaggtttcctggcacgggtaagaattgtggaaagctagtatgcagttaatgcaa |
| | | gggcgcgtttttcgctaccccgacactgctaaagtttttgggaggggtcccttaaacatttctagtatt |
| | | gagtgatagctttgcggcaggtcaccacaaccttactataaataaacctgttgaatctcac |
| 480 | Porcine | tacgcatgtattccacactcatttcccccctccacccttaaggtggttgtatccccataccttaccctcc |
| | Sapelovirus | cttccacaatggacggacaaatggatttgacctcacggcaaacacatatggtatgatttcggataca |
| | JD2011 | ccttaacggcagtagcgtggcgagctatggaaaaatcgcaattgtcgatagccatgttagtgacgc |
| | | gcttcggcgtgctcctttggtgattcggcgactggttacaggagagtaggcagtgagctatgggca |
| | | aacctctacagtattacttagagggaatgtgcaattgagacttgacgagcgtctcctcggagatgtg |
| | | gcgcatgctcttggcattaccatagtgagcttccaggttgggaaacctggactgggcctatactacc |
| | | tgatagggtcgcggctggccgcctgtaactagtatagtcagttgaaaccccccc |
| 481 | Porcine | ttgaaatgggtgtggggtacatgcgtattacggtacgcatatattccacactcatttccccccctcca |
| | Sapelovirus A2 | cccttaaggtggttgtatccccataccttaccctcccttctaaaacagatggacaaatggatttgaact |
| | | tatggcaagtgaatatggtatgactttggatacactttaacggcagtagcgtggcgagctatggaaa |
| | | aatcgcaattgtcgatagccatgttagtgacgcgcttcggcgtgctcctttggtgattcggcgactgg |
| | | ttacaggagagtaggcagtgagctatgggcaaacctctacagtattacttagagggaatgtgcaatt |
| | | gagacttgacgagcgtctcttagagatgtggcgcatgctcttggcattaccatagtgagcttccagg |
| | | ttgggaaacctggactgggcctatactacctgatagggtcgcggctggccgcctgtaactagtata |
| | | gtcagttgaaacccccc |
| 482 | Simian | ccaaggatctgttgcataggcgttgtatcccctaaccttttacctacccatcccaataggactggtatt |
| | Sapelovirus 1 | tcggttttgattgagtaatggatactgattctatacctgttacccattcaggggaaaaatggagtttcttt |
| | | catggatctgacttgatatgaccaagagtcaacactttgcgtgttggccgtatggaatgctttaaggtt |
| | | tattctttggattatgacttcagggttggccgcccaggataaaaggcaattgtggtaagtgatgttagt |
| | | cattggtggttgaaacctgcctaagacgtcctaggtctacgctgtgcgggccgaagtaagcttagg |
| | | aataacagggagtatgccattttctgctttcacccaacacgaccgtacacgaaagagctagaggca |
| | | ctttggggcaaagggaaaagctttgcttagcccgaatgttcatttgagtccttgacgaatgcgtccc |
| | | gtctgtcccgacggtgaggcgtatggcgcatgctcatggcattacccaatggtgtatctgtgaggg |
| | | gggggctcctcacacttagtctagtgctacctgacagggccgcggctggtcgtttgtgtatggtata |
| | | accagtagtaatcccccatggattgctttaacttcccctcctcccttaccaagacattctctaag |
| 483 | Simian | ttttaacttgttatgacattcaaggaaaaaatgtctttttcattatgggactgacctgtttatgaacatgag |
| | Sapelovirus 2 | cagcggcactgctccacgggctatccgtgtaagaaatattgattattcttatggatcatgatttcagg |
| | | gttggccgcccagtctaaaaggcaattgtggtaagctatgtaagtagttggctgttgaaaggagcc |
| | | aagtacatcctaggtctacgctgtgcgggccgaagtaagacttggaacaactctgagtaggcagtt |
| | | tttctctttagcccaacacgaccgcatactgaagagctagaggcactttggggcaaaggtaaaagc |
| | | attgcttagaccgaatgttcaatgagaccttgacgagtgctgtcacagtgtcccctgatggcagtatg |
| | | gcgcatgctcttggcattacccatatgtgtatctatagggggggggccccctatacttagtctagtgc |
| | | tacctgacagggccgcggctggtcgtcggtgtgtggtataaccagtagtaatcccccatggattgc |
| | | tttaactccccctcctccctcaacaaaactttctctaag |
| 484 | Rabovirus C | ccgggtataacccggagttttggggcaggtccaagccccacataggaacatacgatccacggatc |
| | | gtgtgttcttttatgctttctaaccttaccctttgtaaccattacgctttacgccgcatggtgtttggcggc |
| | | accatgacgtggacaagaggttacgccattacgatatgtaccctccccttttggggagagaccgac |
| | | caattatggtacagtatccaactgtattgtggtcaagtacttctgtttccccggtgatgcgggataggc |
| | | tgtacccacggccaaaacctgctgatccgttacccgactcacatctacgaggaggctagtaaaag |
| | | gcatgaagttcaagagtatgatccaaccagatccccactggtaaactagtgatgagggttcccgttc |
| | | cgaacatggcaacatgtgggttccctgcgttggcactaggccccttccgaggggtgctctgaagat |
| | | ggattgttgatgaagaccaatttgtgcatgtgtttatcctccggccctctga |
| 485 | Rabovirus A | ccgaccccactggtcgaaggccacttggcaataagactggtggaacaaggtcgcctgtagttgatt |
| | NYC-B10 | ggaaccttctttctaatgacttatgtcagcggtgctactcacaccgtaactctcctaccctatccccac |
| | | gcttgtggaactaggaggggatgagtgattcaagtaagtactgtcagaatggtgaaaataatctgat |
| | | tctgaaacgctatggatccatcgaaagatggggctacacgcctgcggaacaacacatggtaacat |
| | | gtgccccaggggccgaaagccacggtgataggatcacccgtgtagtttgagatcatatcaatgttc |
| | | atagtctagtaagatgatttgaaatctaactggtctgatggctaactgcttgtcttattgcggcctaagg |
| | | atgtcctgcaggtacctttagagaaccttaagagactattgatctgagcaggagccaaggtggtcttt |
| | | cccagccttggttaaaaagcgtctaagccgcggcagggggcgggaggccccctttcctcccaaa |
| | | ctataatatagattgt |
| 486 | Parabovirus C | gatgtatccccatcccccagtgtgtatgccatactgcatagctcgcctatgccctatggattcacaac |
| | | cctttcatataccctccctacccaaccccgtaaccacatgctttactccgcttggggttttgcggcccc |
| | | atgttgtgacgaaatggctacgcaatcaatgcggctaatggggcctgccgcttttaagtggcccca |
| | | gttagaagtttatgcacacccgcccattaggaggccaccagccaggtggtcagagggcaagcac |
| | | ttctgtttccccggtgaagtttgataagctgtgcccacggctgaagcagacagatccgttacccgcc |
| | | tcactactacgagacggctagtagtgtgtaatatccgaatttcattgatccgggtgttccccccaccc |
| | | agaaacgtgtgatgaggagcggcacccctcctatggcaacatagggcctctcctgcgctggcac |
| | | acgggctctatgagcatgaaatcaggagaaagtcacacgaagaccttattgtgctagtgttgattcc |
| | | tccgcccccctgaatgcggctaatcccaactccggagcgcccgctggcaaacccgccagaaga |
| | | gcgtcgtaatgcgtaagtctggagcggaaccgactactttgggtgtggcgtgtttcctttatttccttt |
| | | gtatttgtat |
| 487 | Parabovirus B | aacccataatccattgtccatcaatgttttatgggggggaccctttctcccctccccctccaaatacct |
| | | tttacccctctgtaaccaagagtgtgcaaaatctatttactagcccagaattgcggcttctggggagg |
| | | tttattcctcatgcctaacaagatgttacgcaaactccgggctacggccctgggcttttgccctaaag |
| | | atttagaagtttacactatcgtccaacaggaggacaacaaaccagttgttctaaggacaagcacttct |
| | | gtttccccggtgagactggatagactgtacccacggttgaaactggttgatccgttacccgactcac |
| | | tacttcgagaagattagtaggaaactgtgaaactgattccattgatccggatactttccccgtatcca |
| | | gaaactactgatgagggttgacttcccgactacggcgacgtagtgtcatccctgcgctggcagtag |
| | | gcctctttgaggatggaagatgtggatcggtaaccgaaggtcctattgagctagtgtttatacctccg |
| | | gcctcctgaatgcggctaatcctaacccatgatctagtgctcacaaaccagtgagtagctagtcgta |
| | | acgcgtaagtcgtgggcggaaccgactactttggagtgaccgtgtttcctattttacttttgtttg |
| 488 | Parabovirus A3 | accgttacgcaccactcagttggtgtttggtggcaccaatgatggaacaaaaggctacaccacttg |
| | | ggctacggcccgcgccaccttgtggcgcaaagacattagaagaatagcataccgcccactaggg |
| | | ccctgcagccagcagggtaacgggcaagcacttctgtctccccggtagaacggtataggctgtac |
| | | ccacggccgaaaactgaactatcgttacccgactccgtacttcgcaaagcttagtaggaaactgga |
| | | aagttcgagttattgacccggagtgttccccccactccagaaacgcgtgatgagggttgccacccc |
| | | gaccatggcgacatggtgggcatccctgcgctggcacgcggcctctaagaggataactcgctcct |
| | | actggtaaccgaagagccccgtgagctacggtttattcctccgcctccctgaatgcggctaatccta |
| | | acccatgagcagttgccatagatccatatggtggactgtcgtaacgcgtaagttgtgggcggaacc |
| | | gactactttgggatggcgtgtttccttgttttctccatttgttgttgtatggtgacaagttatagatctcga |
| | | tctatagcgtttcttgagagatttccaaacatttattcaagtcgtacaattcttgtgtttaagcagtacagt |
| | | gtaagg |
| 489 | Felipivirus 127F | gatgtcggatgacggctggccaccggggaaaaacggcaaatgtgcaccacctctgcaacccac |
| | | gccgaccacgtttaaccatggcgttagtaggagtggaccactgcagtgggctctggtgtgcgaca |
| | | gtcagtggtagagtagacagtcctgactgggcaatgggaccgcgttgcgtatccctaggtggcat |
| | | cgagattcctctgctacccaccagcgtggactcctatggggggggccccataggctaggtctatac |
| | | tgcctgatagggtcgcggctggtcgaccactgactgtataaccagttgtaactcact |
| 490 | Boosepivirus A | ttgaaagacctcggcatatatcgttgtcacaacggtatatgtcgagatctttctccccaccccctcca |
| | | attcccttttccccctcttgcaacttagaagtttgtacacacagggcaataggatacgtgatccagcc |
| | | aggacacgtgagctcaagcacttctgtttccccgtccccttcacgtactacgggaatgttagtaattt |
| | | gtgtgcactttagtaaggttgatccgggattaaccccaaatcccagaaactggtgatgagcgttacc |
| | | acccccgccgggcgaccggaaggtttcgctgcgttggcaccagggcttcggcaccagaaaaag |
| | | gtaaagcaaatgaaggcgctactgtgctacgagaagtttcctccaggcccctgaatgcggctaatc |
| | | ctaaccagtgatccaccggtgcaaaaccatgtactaggtggtcgtaacgcgcaagtcgctggcgg |
| | | aaccgactactttgggtgtcctgtgtttccatattttattttattcaattttatggtgacaagagtaaagag |
| | | atacagatttgcagcc |
| 491 | Boosepivirus B | ttttctcccctccccctccaactaccttttccccctcttgtaacgctagaagtttgtgcaaaccgcctgt |
| | | agggtactgcaatccagcagtgcataggctaagcttttcttgttaccccaccccacattatactgagg |
| | | aggattgtgaaattgtgttagtatgggttagtagcggtgacccgggtaaccccaacccagaaactc |
| | | acggatgagatgaacaggaccccacatggtaacgtgtgtgttcgtctgccccgcaaggtgaggcc |
| | | gtgagagctttgcacgcgaaaaccttgaaaacccaaaagtaccttgagctcttcgctattttgtgtttc |
| | | ctccaggaccctgaatgcggctaaacctaacccgcgatccgcacgtagcaacccagctagagtgt |
| | | ggtcgtaatgcgcaagttgcgggcggtaccgactactttggtgttcctgtgtttcctttattttattttga |
| | | atttttatggtgacaacagctagaaaataagagtgaac |
| 492 | Phacovirus Pf- | gtgtgtcatttctcccctccccctcccaaaccttttccccctctaatcggattgattaacccggttaaag |
| | CHK1 | atgattaatggtttgtgagttgatatgatggcccggcattgaatccgggaattcttaagtaatggaatt |
| | | gcatccaatatgaaagtgagtgtggcaagctcacaagtagtacttggctctgcccattatttgagga |
| | | ccaactcttcttgactacaatgtgtttaaagtaaactggaccacattgtgtatccagacaactccatttg |
| | | ataatgtacgctggaaacgttttcagtgcatagggtcctaaagtggtgctgaaatattgcaagctcaa |
| | | tgggatactgaacgctgaaaaccgccgctgttatcatatgggcccctagtgggtaaatgttggcttt |
| | | aggcatatactgcttgggaatgcagtactggttgtagacagggtgatagcctaccggctggcgtag |
| | | ttgagttggtatagccagttgattgccat |
| 493 | HRVC3 QPM | ttaaagctggatcatggttgttcccaccatgattacccacgcggtgcagtggtcttgtattacggtac |
| | | atttccataccagttttatacaccccaccccgaaactcatagaagtttgtacacaatgaccaataggt |
| | | ggtggccatccaggtcgctaatggtcaagcacttctgtttccccggcacccttgtatacgcttcaccc |
| | | gaggcgaaaaatgaggttgtcgttatccgcaaagtgcctacgaaaagcctagtaacactttgaaaa |
| | | cccatggttggtcgctcagctgtttacccaacagtagacctggcagatgaggctagacattcccca |
| | | ccagcgatggtggtctagcctgcgtggctgcctgcacaccctgccgggtgtgaagccagaaagt |
| | | ggacaaggtgtgaagagcctattgtgctcactttgagtcctccggcccctgaatgtggctaacccta |
| | | accccgtagctgttgcatgtaacccaacatgtatgcagtcgtaatgggcaactatgggatgggacc |
| | | aactactttgggtgtccgtgtttcctgttttactttttcattgcttatggtgacaattgtatctgatacacttg |
| | | ttacc |
| 494 | HRVB27 | ttaaaacagcggatgggtatcccaccatccgacccacagggtgtagtgctctggtattttgtaccttt |
| | | gcacgcctgtttccccattgtacccctccttaaatttcctccccaagtaacgttagaagtttaaggaaa |
| | | caaatgtacaataggaagcatcacatccagtggtgttatgtacaagcacttctgtttccccggagcg |
| | | aggtataagtggtacccaccgccgaaagcctttaaccgttatccgccaatcaactacgtaatggcta |
| | | gtattaccatgtttgtgacttggtgttcgatcaggtggttccccccactagtttggtcgatgaggctag |
| | | gaactccccacgggtgaccgtgtcctagcctgcgtggcggccaacccagcttttgctgggacgcc |
| | | tttttacagacatggtgtgaagacctgcatgtgcttgattgtgagtcctccggcccctgaatgcggct |
| | | aaccttaaccccggagccttgcaacataatccaatgttgttgaggtcgtaatgagtaattctgggatg |
| | | ggaccgactactttgggtgtccgtgtttccttttattctttatattgtcttatggtcacagcatatatagcat |
| | | atatactgtgatc |
| 495 | HRVA73 | ttaaaactgggtttgggttgttcccacccaaaccacccacgcggtgttgtacactgttattccggtaa |
| | | ccttgtacgccagttttatatcccttcccccccttgtaacttagaagacatgcgaatcgaccaatagca |
| | | ggcaatcaaccagattgtcaccggtcaagcacttctgtttccccggctctcgttgatatgctccaaca |
| | | gggcaaaaacaattggagtcgttacccgcaagatgcctacgcaaaacctagtagcatcttcgaag |
| | | atttttggttggtcgctcagttgctaccccagcaatagacctggcagatgaggctagaaatacccca |
| | | ctggtgacagtgttctagcctgcgtggctgcctgcacacccacacgggtgtgaagccaaagattg |
| | | gacaaggtgtgaagagtcacgtgtgctcatcttgagtcctccggcccctgaatgcggctaacctta |
| | | accccgtagccattgctcgcaatccagcgagtatatggtcgtaatgagtaattacgggatgggacc |
| | | gactactttgggtgtccgtgtttcactttttacttatcaatttgcttatggtgacaatatatatagatatatat |
| | | tgacacc |
| 496 | EV L | acatgggccagcccaccacacccactgggtgtagtagtctggttctatggaacctttctacgcctctt |
| | | ttgcttccctcccccatttctccttcgattgctccacctgtgatctttgcaacttagaagaaataatgaac |
| | | ccgcacaatagcgggcgctgagccacagcgtcaatgtgcaagcacttctgtttccccggaatggg |
| | | cccataggctgtacccacggctgaaagggaccggcccgttacccgccttggtactgcgagaatgt |
| | | tagtaactccctcgatagctttaggcgttacgctcagccctttgagcccgaagggtagttcgggtcg |
| | | atgaggctcgtcattccccactggcgacagtgtgacttgcctgcgttggcggcccggggtggggg |
| | | gcaacccccatccacgcctactgaaggacagggtgtgaaggcgctattgcgctactaaggagtcc |
| | | tccggcccctgaatgcggctaacccgaaccccgagcccacggtggtaaacccgccacaagtgg |
| | | gtcgtaatgagtaatttggggcagggaccgactactttgggtgtccgtgtttcctgtttttccatacgat |
| | | ggctgcttatggtgacaaccataagcaattggattggccatccggtgttcatattgcgaat |
| 497 | EV K | tcagcctgacgcaagtgcctccattggagtctctccaagccctccggggcttggagggcgccgac |
| | | cccctgcctagggggagcccacgacacggctggagtccattggcacaccgcagccacgattca |
| | | agccagaattgaaagcgggaagcacttctgtctccccggtgtggatcatacgctgtacccacggc |
| | | gaaaagtgaagcatcgttacccgactcggtacttcgagaagcccagtacagttgtggatctctgca |
| | | gggtatacgctcagcgtgacccctacgtagttccttgagatggctgagagaacaccccacgggcg |
| | | accgtgtctctcggcgcgtggctcaaggccgggccttcagtggctcggtgccttgcagagtgaag |
| | | cctccgaacagcctattgagctaccgtttagcctccgccctcttgaatgcggctaatcctaaccatg |
| | | gagcgcccgcccacagtccagtgggtagagcgtcgtaacgcgcaagtccgtggcggaaccgac |
| | | tactttagagtggcgtgtttccaatttatcctttataaagttgcttatggtgacaccacaagagatccac |
| | | gatttcttgtttcttatcactgagacacaagtcatattcatcaatctttattgcggaattaacttggtgcgt |
| | | ccaaacacatcagc |
| 498 | EV J 1631 | caccctgagggcccacgtggcgtagtactctggtatcaaggtacctttgtacgcctattttatttccct |
| | | tcccccacagtaacttagaagcttatctcatagttcaacagtagggtcactaaccaagtggctcagc |
| | | gaacaagcacttctgtttccccggtcctagtacctgtgaagctgtacccacggcggaaggggaaa |
| | | aagatcgttatccggccccctacttcggaaagcctagtaacaccattgaagcaatcgagtgttgcg |
| | | ctcagcacagtaacccctgtgtagctttggttgatgagtctgggcactccccactggcgacagcgg |
| | | cccaggctgcgttggcggccaaccgactcgggcaaccgggtcggacgctcgtttgtggacatgg |
| | | tgtgaagagcctactgagctagagggtagtcctccggcccctgaatgcggataatcctaaccccg |
| | | gagcacccacactcaatccagagtgcaggatgtcgtaacgcgtaagtctgggacggaaccgact |
| | | actttgggtgtccgtgtttcctgttttacttactttggctgcttatggtgacaatctagtgttgttaccatat |
| | | agctattggattggccatccggtgttttgaattgtgtgtttatactaattcttttacatatcacagacaacc |
| | | aaat |
| 499 | EV J N125 | cggtacctttgtacgcctattttacccccttccccttgtaacttagaagcaaagcaaaccagttcaata |
| | | gtaagcaacacaacccagtgttgtgacgaacaagtacttctgtttccccgggagggtctgacggta |
| | | agctgtacccacggctgaagtatgacctaccgttaaccggctacctacttcgagaagtctagtaata |
| | | ccattgaagttttgttggcgttacgctcaacacactaccccgtgtgtagttttggctgatgagtcacgg |
| | | cattccccacgggcgaccgtggccgtggctgcgttgcggccaaccaaggggcgcaagctccttg |
| | | gacgtcacttaacagacatggtgtgaagaacctattgagctaggtagtagtcctccggcccctgaat |
| | | gcggctaatcctaactccggagcacatcagtgcaacccagcatttggtgtgttgtaatacgcaagtc |
| | | tggagcggaaccgactactttgggtgtccgtgtttcctgttttaccttatttggctgcttatggtgacaa |
| | | tttgatattgttaccatatagctgttggattggccatccggatttttgaaagagacccaaaactttcttct |
| | | ctacttcagattcaagtgcgaagttttccttttcatatattacttactaatttgaagtaccaaag |
| 500 | EV I | ttagtactttctcacggggatagtggtatccctccctagtaatttagaagacttgaaaaaccgaccaat |
| | | aggcacctcgcatccagcggggtaaaggtcaagcacttctgtttccccgggtcgagtagcgatag |
| | | actgtgcccacggtcgaaggtgaaacaacccgttatccgactttgtacttcgggaagcctagtacc |
| | | accaaagattatgcttggggtttcgctcagcacgaccctggtgtagatcaggccgatggatcaccg |
| | | cattcctcacaggcgactgtggcggtggtcgcgtggcagcctgccgatggggcaacccatcgga |
| | | cgccaagcatatgacagggtgtgaagagcctactgagctacaaagtattcctccggcccctgaat |
| | | gcggctaatcccaaccacggagcatttgctaccaaaccaggtagtggaatgtcgtaacgggtaac |
| | | tctgtggcggaaccgactactttgggtgtccgtgtttccttttaatttatcattctgtatatggtgacaact |
| | | atagtgctatctcgatttgcattactattgttgagattaaaactttattacattgttgcattttaccctttgag |
| | | tgagttttcacctgaacagattaatttactcatcctgtttatatattacaagcagaaatacttgcaaag |
| 501 | EV F1 BEV 261 | gcaatgctgcaccagtgcactggtacgctagtaccttttcacggagtagatggtatcccttaccccg |
| | | gaacctagaagattgcacacaaaccgaccaataggcgcaccgcatccagccgtgcagcggtca |
| | | agcacttctgtctccccggtctgtaaagatcgttatccgcccgacccactacgaaaagcctagtaac |
| | | tggccaagtgaacgcgaagttgcgctccgccacaaccccagtggtagctctggaagatggggct |
| | | cgcaccacccccgtggtaacacggttgcctgcccgcgtgtgcttccgggttcggtctcgtgccgtt |
| | | cacttcaacttcacgcaaccagccaagagcctattgtgctgggacggttttcctccggggccgtga |
| | | atgctgctaatcccaacctccgagcgtgtgcgcacaatccagtgttgctacgtcgtaacgcgtaagt |
| | | tggaggcggaacagactactttcggtaccccgtgtttcctctcattttatttaatattttatggtgacaat |
| | | tgttgagatttgcgctcttgcaacgttgccattgaatattggcttatactatttggttgccttttacaaaac |
| | | ctctgatatacccagttcttacattgatctgcttgtttttctcaatttgaagtatagactacaaatagcaaa |
| 502 | EV D94 | cgtggcggccagtactctggtatcacggtacctttgtacgcctgttttatatccccttcccccgcaact |
| | | tagaagaaaacaaatcaagttcactaggagggggtacaaaccagtaccaccacgaacaagcact |
| | | tctgtttccccggtgatgtcgtatagactgtaaccacggttgaaaacgattgatccgttatccgctctt |
| | | gtacttcgaaaagcccagtatcaccttggaatcttcgatgcgttgcgctcagcactcaaccccagag |
| | | tgtagcttaggtcgatgagtctggacactcctcaccggcgacggtggtccaggctgcgttggcgg |
| | | cctacctgtggtccaaagccacaggacgctagttgtgaacaaggtgtgaagagcctattgagctac |
| | | aagagaatcctccggcccctgaatgcggctaatcctaaccacggagcaagggtacacaaaccag |
| | | tgtatatcttgtcgtaacgcgcaagtctgtggcggaaccgactactttgggtgtccgtgtttccttttgt |
| | | ttttatcatggctgcttatggtgacaatctaagattgttatcatatagctgttggattggccatccggtaa |
| | | tttattgagatttgagcatttgcttgtttcttcaacaatttcacctattcattgcatttcagcagtcaaa |
| 503 | PV3 | tacctttgtacgcctgttttatactccctcccccgcaacttagaagcatacaattcaagctcaatagga |
| | | gggggtgcaagccagcgcctccgtgggcaagcactactgtttccccggtgaggccgcatagact |
| | | gttcccacggttgaaagtggccgatccgttatccgctcatgtacttcgagaagcctagtatcgctctg |
| | | gaatcttcgacgcgttgcgctcagcactcaaccccggagtgtagcttgggccgatgagtctggac |
| | | agtccccactggcgacagtggtccaggctgcgctggcggcccacctgtggcccaaagccacgg |
| | | gacgctagttgtgaacagggtgtgaagagcctattgagctacatgagagtcctccggcccctgaat |
| | | gcggctaatcctaaccatggagcaggcagctgcaacccagcagccagcctgtcgtaacgcgcaa |
| | | gtccgtggcggaaccgactactttgggtgtccgtgtttccttttattcttgaatggctgcttatggtgac |
| | | aatcatagattgttatcataaagcgagttggattggccatccagtgtgaatcagattaattactcccttg |
| | | tttgttggatccactcccgaaacgttttactccttaacttattgaaattgtttgaagacaggatttcagtgt |
| | | caca |
| 504 | EV C102 | ctttgtacgcctgttttacatcccctcccccacgtaactttagaagcaattcaacaagttcaatagagg |
| | | gggtacaaaccagtatcaccacgaacaagcacttctgtttccccggtgattttacataagctgtgcc |
| | | cacggctgaaagtgaatgatccgttacccgctcgagtacttcgaaaagcctagtatcgctttgggat |
| | | cttcgacgcgttgcgctcagcactctaccccgagtgtagcttaggctgatgagtctgggcattcccc |
| | | atcggcgacgatggcccaggctgcgttggcggcctacccatggctaacgccatgggacgctagt |
| | | tgtgaacaaggtgtgaagagcctattgagctactcgagagtcctccggcccctgaatgcggctaat |
| | | cccaaccacggatcaggtgcctccaacccaggaggtggcctgtcgtaacgcgcaagtctgtggc |
| | | ggaaccgactactttgggtgtccgtgtttccttttatcttttaaatggctgcttatggtgacaatcataga |
| | | ttgttatcataaagcgaattggattggccatccggtgaaatacaaacacattatttacttgtttgttggat |
| | | ttactccgctcacacagcttactcctaagataatatttattgtattgctggtaaggagacactattata |
| 505 | EV 30 | aagcaaggcaaacctgaccaatagtaggtgtggcacaccagccgcattttggtcaagcacttctgt |
| | | ttccccggaccgagtatcaataagctgctcacgcggctgaaggagaaaccgttcgttacccgacc |
| | | agctacttcgagaaacctagtaacactatgaacgttgcggagtgtttcgttcagcacttcccccgtgt |
| | | agatcaggtcgatgagtcaccgcattcctcacgggtgaccgtggcggtggctgcgttggcggcct |
| | | gcctacgggttcgcccgtaggacgctctaataccgacatggtgtgaagagtccattgagctagctg |
| | | gtagtcctccggcccctgaatgcggctaatcctaactgcggagcaggtgctcacagaccagtgag |
| | | tagcctgtcgtaacgggcaactctgcagcggaaccgactactttgggtgtttttccttttttcttctctta |
| | | tattggctgcttatggtgacaattaaagaattgttaccatatagctattggattggccatccggtgacg |
| | | agcagagccattgtttacctctttgttggatttgtacctttgaaccacaaagtcttgaataccattcatct |
| | | cattttaaagttcaactcagctaaaagaaa |
| 506 | SA5 | agtacttggtattccggtacctttgtacacctatttacaaaccctaccccttgtaaccttagaagcaatt |
| | | atttaaccgctcactagggggtgtgctatccaagcacatcaagagcaagcacttctgtctccccgg |
| | | gaggggctaatggtacgctgtgcccacggcggaaatgagccctaccgttaaccggcagtctactt |
| | | cgggaagcccagtaactacattgaaactttgaggcgttacactcagcacataaccccaatgtgtagt |
| | | tctggtcgatgagccttggcatcccccacaggcgactgtggccaaggctgcgttggcggccagc |
| | | ctgcggaccaaaagtccgtaggacgcctaattgtggacatggtgtgaagagcctactgagctaga |
| | | ctgtagtcctccggcccctgaatgcggctaatcctaaccctggagcatccgcgtgcaacccagtac |
| | | gtagggtgtcgtaatgcgtaagtctgggatggaaccgactactttgggtgtccgtgtttcttgtttttca |
| | | tactgggtcgcttatggttacaactaattgttgtaatcattggcagtgcgcgctgaccacgcgattatt |
| | | gatatttccatttgttggatactccaatagtgtcaactcatatacacaacttttaccactgatcaagataa |
| | | aa |
| 507 | EV A114 | tgtgcgcctgttttgaaaccccctcccccaactcgaaacgtagaagtaatgtacactactgatcagta |
| | | gcaggcgtggcgcaccagccatgtctcgatcaagcacttctgtttccccggactgagtatcaatag |
| | | actgctcacgcggttgaaggtgaaaacgtccgttacccggctaactacttcgagaaacctagtagc |
| | | accatagaaactgcagagtgtttcgctcagcacttcccccgtgtagatcaggtcgatgagtcactgc |
| | | aatccccacgggtgaccgtggcagtggctgcgttggcggcctgcctatggggcaacccatagga |
| | | cgctctaaggtggacatggtgtgaagagtctattgagctagttagtagtcctccggcccctgaatgc |
| | | ggctaatcctaactgtggagcgcatactcccaaaccagggagcagtgcgtcgtaacgggcaactc |
| | | cgcagcggaaccgactactttgggtgtccgtgtttccttttattcctatactggctgcttatggtgacaa |
| | | ttgagagattgttaccatatagctattggattggccatccagtgtgtaatagagcaatcatttaccaatt |
| | | tgttggatttactccattaacccacacgtctctcaacacactacatttcatcttactactgaacactaga |
| | | aa |
| 508 | Mobovirus A | tattctcccacaaaccttcttgtaactctgttaagccttttacatccatgtaatttaattttctccacctaaa |
| | | aggatttcccccatggtcctttttggctcgaacaaatgctacatagggtcttgttttctccccctggctc |
| | | tcttgccagggttccataccccaattcctctatttccatgatttttcatcatggtttatttttactgtcttctta |
| | | ttttctgaggtgaccaactcctaagccgactgggtcgcggaagcccggactcctcgcatcactagg |
| | | gtgcgtagcgatgtaggcgaaaatattggttgctagatgcatacatatagtgaattgatactacacca |
| | | aactctgttctttttgaaactagctattttctaagtaaggtaggctacgggtgaaaccttaccattgcag |
| | | gtacgtgaaccgcaacggacatttggccgaagactggtgtacccacgtcagttataggacctcttc |
| | | aacgttggtggacggcatgtcactgattagttaggctagtgaatttaagttcagggggtatcttttagc |
| | | ttaagcgtgtattctagtaggacttgcagagcctccccacctaggaggatctctgtttatagccccttt |
| | | tccttgttccgttagttttccacacttttacaatatttgatgatttgtt |
| 509 | Burpengary Virus | ctccccccccttccccttcccgagtaggagattggcatgtatgctctacatgcccgattctctcttgct |
| | | cactctcttaaatcctggtggcggtctcggattaaacatttatgtcgtatctgggatcgtcttacttggt |
| | | ggtaattcctctgttgcctagggacctccggactgccggattaaaggtctcaacagagggcaatgt |
| | | acaaggaagtcattatacgctaattaagtatttgatgaatgactagtgtgacagggctgaggaactc |
| | | cccccgggtaaccggtgcctcagcgtccgaaagacacgtggataggatccaccctgttataccca |
| | | gcacgatgtaatagtcaaatacctctgatttgtgtaggatgtataaattgtgcattgtaaattttgggcg |
| | | tagagatgctccgaaggtaccccgttttacgggatctgatcggaggctaattacccaatgcgcccta |
| | | aataacttcatataatttctttttctttattcaaa |
| 510 | Hunnivirus A1 | taacgtttggcaagaaccctcacctgtcaattgggaccaccactttcagtgaccccatgcgaagtag |
| | | tgagagagaataagctttcttacccttcatttgtgaacccttcagtcgaagccgcttggaataagata |
| | | ggaggaaaagttcattctaaatggagtgaaacatgtacttcagaatttctagcacgcgctgggctttc |
| | | ttgcgtgtgacggcactgtcttgccggagctctccacactgacaccccacgcttgtggaccttggtg |
| | | gcagatgacaacactgcagctggaattgagtgtctggtacactctgtgtaacagtgaaaacaatgt |
| | | gatcacttcggtgagctagtagcctgtggaccaacaactggtaacagttgcctcaggggccaaaa |
| | | gccacggtgtttacagcaccctactggtttgattggagcaatccaagatgtcacagagttagtaattg |
| | | ccaagcagtccgtactggtatcttgacataccgtgcagttttggatagtgaaggatgccctgacggt |
| | | acccataggtaacaagtgacactatggatctaagcaggggctcactctacgctgctttacagctgg |
| | | ctgtgagttaaaaaacgtctagctatccacaacctaggggactaggttttccttttatttagattacaatt |
| | | at |
| 511 | Hunnivirus A2 | acagtttttgacaaggaccctcacctgtcaatcgggaccaccactttcagtgaccccgtgcgaagt |
| | | gttgagagaaagtgagctttcttacccttcatttgtgaacccttcagtcgaagccgcttggaataagat |
| | | ggaaggaaatgttcattctaaatggagtgaaacatatacttaatttccagtgtttagtggtctttccact |
| | | agacaacggcactgtcttgccggaactctacacaccaacattccacgcttgtgggactcaaatgttg |
| | | gatgacacagttgtagctggaactgagtgtttagtgcactctgtgtaacagtgaaaacaatgtgatca |
| | | cttcggtgggctagtagcctgtggactaacaactggtaacagttgcctcaggggccaaaagccac |
| | | ggtgttaacagcaccctactagtttgattggagcaatccatgatgttacagagttagtaactgccaaa |
| | | cagattgtactggtatcttggcataccgtgcaacttaggatagtgaaggatgccctggcggtaccca |
| | | taggtaacaagtgacactatggatctaaacaggggctcactctacgttgctttacaactagctgtga |
| | | gttaaaaaacgtctaactatccacaacctaggggactaggttttcctttttatttttatacacaacta |
| 512 | Ia Io | ggtgtccgggtgtgtgggatagacccagatgtgcagtggatgcgagcatttgagtcagagtagga |
| | | gcaagcccaggggcaaagggaccacattgtgtatcccgaatgaaggatcgagatttctctcctcat |
| | | tacccggtgtcttgtcactgttgggggggcccaacagtcttagtcctatactgcctgatagggtcgc |
| | | ggctggccggactcaagtgctatagtcagttgattttcactc |
| 513 | Taura Syndrome | ctttaaaagtcgtgcgtggcttcaccacgcacgatcagtactatcagttaaccactcttgaatatgctc |
| | Virus | aatgaccctattcaacactggtgtctcttagtacattattttagcacttaacgtgcatgagttttgcccat |
| | | ttctttcaaaaaaatgagtattcgaggagacgtcccgctccccgtcttatttcaaccgtagactcgac |
| | | atctattggtggacatttaattccagtcgccgtaagttgcttctgccccgcgctatattttcttatacttat |
| | | ggttctataggtctggtttaaaacgtaaatagacggcccacaaactatagaacgcgtacccggaac |
| | | gccaatcccggataagtccctggatatatagatgcaccgcaatataagcctgcagactgtctcatat |
| | | act |
| 514 | ABPV | cccgtcaaaataacaacttataacacgatgttacccgaagaaaccattttagtgtaacatttaagatta |
| | | gaagtagttcatctaatagagataggcactattagaaggaggccttttctaaaggagccgttagtca |
| | | gcccagacaagcgcagtactttagaagagagaagttccccgatagcgaccgaaaagacgcgtttt |
| | | ccgtgctaactaatttaaatgtgggaacgaatattattattgaaattatgtgagccacgtagcaatcaa |
| | | gtcatgtttttgtcactacgtttactcatctaatgtagataattttgtttaagtacctatttaggtgtcatccc |
| | | accagagaagaaataatacgtaccggaacccagagtacaccccttattttaagccttactgggcttc |
| | | tctgttagttagtaatctggcccacgttttgcgttgagtggggtcccaacagtaggaattcgacggac |
| | | aagtagcaagcgagtcggtaccaattggtttagccttcgaaattactctgggcaggaagttactaaa |
| | | cgagaactttctgcttaaatcccaacgcacaaacaaatagagtaaataaataattata |
| 515 | BRAV-2 | tttgttttgcggctttgccgttgttcgggttttacctgttttcacacagcaaaacaggccttctagtttcgt |
| | | gcttaaacgagatcatgctcgaactagaactacatagctggtcactggactcataccacaccttgtg |
| | | gagctttatgggaaaggtggctagtgggctgtggaagtgactctgaccacatgcctctcaagtgtg |
| | | ggaaatcacggatcggtgtagcgacgacaacaggccttgggacaccctctccagtaatggagac |
| | | ccaaggggccaaaagccacgcctcgtgccctgttgttcacaaccccagtgcgacccgtgttagta |
| | | cctatttgcgagaactgtgtctggacagctaaacacaaccctagtgggagactaaggatgcccag |
| | | gaggtacccggaggtaacaagtgacactctggatctgacctggggagagagggcttgctttacag |
| | | gcgcctctctttaaaaagcttctatgtctcatcaggcaccggaggccgggccttttccttttaaaatta |
| | | cactta |
| 516 | BRBV-1 | ccccccctacttaaagatgtacggttttgctgctttcacagagtaaagcagatagaggttctgaactg |
| | | gcaaactttacctcgaaacacgcccgtttttctgctgtgtctcacagactgtcctgtcacacttgtggc |
| | | ggcttgtgacactgtgaacatagtgagaccgaccaagacaacagtttcaagtgatgaacatcgaac |
| | | gtctaaactggatccgtaactggacatgttagggcaaggacttcccccctggtaacaggagcctgg |
| | | ctggccaaaagccccgctcattgagcctagcatgttgtcgaccctggactgttcagtttagttagtac |
| | | atggaattcacttgtcacggttcttctgaactcggtctctagtatgacagcctaaggatgccctccag |
| | | gtaccccggggtaacaagtgacacccgggatctgaggaggggactactttacgtagtttaaaaaa |
| | | cgtctaagctgttatggtgaccagaggctggcacctttcacttttaaaattacactactgactacaatt |
| | | gaagtgataacggttttacaggctttcaaactagttacacaagcactgttttcctgacacacacacttt |
| 517 | ERAV-1 U188 | aatattggcgcgcgcatttgcgcgcccccccccatttcagccccctgtcattgactggtcgaaggc |
| | | gttcgcaataagactggtcgtcacttggctgttctatcgtttcaggctttagcgcgccctcgcgcggc |
| | | gggccgtcaagcccgtgcgctgtatagcgccaggtaaccggacagcggcttgctggattttcccg |
| | | gtgccattgctctggatggtgtcaccaagctgacaaatgcggactgaacctcacaaagcgacaca |
| | | cctgtggtagcgctgcccaaaagggagcggaactcccccgccgcgaggcggtcctctctggcc |
| | | aaaagcccagcgttaatagcgccttttgggatgcaggagccccacctgccaggtgtgaagtggag |
| | | tgagtggatctccaatttggtctgttctgaactacaccatctactgctgtgaagaatgccctggaggc |
| | | aagctggttacagccctgaccaggggccctgcccgtgactctcgatcggcgcagggtcaaaaatt |
| | | gtctaagcagcagcaggaacgcgggagcgtttcttttccctttgtatcgac |
| 518 | GFTV | atggggaagggtatgacgtgccccttccttcttcggagaactcgctctagtggtctttccacttctgg |
| | | aaaagagtgagtgcacgtgatcaggaccgtcgaagacgacaaatacctggtgctctatctcatag |
| | | acgtttcacagctgtagcgacccctcagtagcagcggaagccccctcctggtgacaggagcctct |
| | | gcggccaaaagccacgtggataagatccactgctgagggcggtgcgaccctagcaccctgtgat |
| | | gcatactagttgtagcgtgccggactattggtctgtcataagacacctgatagagagaccaagaat |
| | | gtcctggaggtaccccgcgtgcgggatctgaccaggagaccattgcccaatgctttacaacgggt |
| | | ctatggtttaaaaactgtcgcagtctctccaaaccaagtggtcttggttttcaattactttgaatatttca |
| | | ct |
| 519 | SAFV V13C | ttttcgacgtggttggaattgccatcatttccgacgaaagtgctatcatgcctccccgattatgtgatgt |
| | | tttctgccctgctgggcggagcattctcgggttgagaaaccttgaatctttttctttggaaccttggttc |
| | | ccccggtctaagccgcttggaatatgacagggttattttcttgatcttatttctacttttgcgggttctatc |
| | | cgtaaaaagggtacgtgctgccccttccttctctggagaattcacacggcggtctttccgtctctcaa |
| | | caagtgtgaatgcagcatgccggaaacggtgaagaaaacagttttctgtggaaatttagagtgcac |
| | | atcgaaacagctgtagcgacctcacagtagcagcggactcccctcttggcgacaagagcctctgc |
| | | ggccaaaagccccgtggataagatccactgctgtgagcggtgcaaccccagcaccctggttcgat |
| | | gatcattctctatggaaccagaaaatggttttctcaagccctccggtagagaagccaagaatgtcct |
| | | gaaggtaccccgcgtgcgggatctgatcaggagaccaattggcggtgctttacactgtcactttgg |
| | | tttaaaaattgtcacagcttctccaaaccaagtggtcttggttttccaattttgttgaatggcaat |
| 520 | SAV P-113 | ggagatctaagtcaaccgactccgacgaaactaccatcatgcctccccgattatgtgatgctttctg |
| | | ccctgctgggtggagcatcctcgggttgagaaaaccttcttcctttttccttggaccccggtcccccg |
| | | gtctaagccgcttggaataagacagggttatcttcacctcttccttcttctacttcatagtgttctatact |
| | | atgaaagggtatgtgtcgccccttccttctttggagaacacgcgcggcggtctttccgtctctcgaaa |
| | | agcgcgtgtgcgacatgcagagaaccgtgaagaaagcagtttgcggactagctttagtgcccaca |
| | | agaaaacagctgtagcgaccacacaaaggcagcggaccccccctcctggcaacaggagcctct |
| | | gcggccaaaagccacgtggataagatccacctttgtgtgcggcacaaccccagtgccctggtttct |
| | | tggtgacacttcagtgaaaacgcaaatggcgatctgaagcgcctctgtaggaaagccaagaatgt |
| | | ccaggaggtaccccttccctcgggaagggatctgacctggagacacatcacatgtgctttacacct |
| | | gtgcttgtgtttaaaaattgtcacagctttcccaaaccaagtggtcttggttttcactctttaaactgattt |
| | | cact |
| 521 | VHEV | aattccttcttcctttctccttggacctcggtcccccggtctaagccgctcggaatatgacagggttatt |
| | | ttcacctcttctctcttctacttcatagtgttctatactatgaaagggtatgtgtcgccccttccttcttgga |
| | | gaacgtgcgtggcggtctttccgtctctcgaaaaacgtgcgtgcgacatgcagagtaacgcaaag |
| | | aaagcagttcttggtctagctctggtgcccacaagaaaacagctgtagcgaccacacaaaggcag |
| | | cggaaaccccctcctggtaacaggagcctctgcggccaaaagccacgtggataagatccaccttt |
| | | gtgtgcggtgcaaccccagcaccctggtttcttggtgacaccttagtgaaccctcgaatggcaatct |
| | | caagcgcctctgtaggaaagccaagaatgtccaggaggtaccccttcctcatggagggatctgac |
| | | ctggagacacatcacacgtgctatacacttgtgcttgtgtttaaaaattgtcacagctttcccaaacca |
| | | agtggtcttggttttcccttaacttcgaaaagtcactatggcctgcaaacatggatacccagacgtgt |
| | | gccct |
| 522 | TRV NGS910 | atgcgacgtggttggagattaaaccgactccgacgaaagtgctatcatgcctccccgattatgtgat |
| | | gttttctgccctgctgggcggagcattctcgggttgatacaccttgaatccttcatccttggacctcag |
| | | gtcccccggtctaagccgcttggaatacgacagggttattttccaatcttctcctttctactttcatgag |
| | | tcctattcatgaaaagggtctgtgctgccccttccttcttggagaatctgcgcggcggtctttccgtct |
| | | ctcgaaaagcgcagatgcagcatgctggaaccggtgaagaaaacagttctttgtggaaacttaga |
| | | gcagacatcgaaacagctgtagtgacctcacagtagcagcggaaccccctcctggtaacaggag |
| | | cctctgcggccaaaagccccgtggataagatccactgctgtgagcggtgcaaccccagcaccct |
| | | ggttcgatggttgttctctgtggaaccagagaatggtctttctcaagccctccagtagagaagccaa |
| | | gaatgtcctgaaggtaccccgcatgcgggatctgatcaggagaccaatcgtcagtgctttacactg |
| | | gcgctttggtttaaaaactgtcacagcttctccaaaccaagtggtcttggttttcacttttatcaaactgt |
| | | ttc |
| 523 | EMCV2 RD1338 | aaatactggtcgaaaccgcttgggataagaccggggtttgttaatgtctcaatgttattctccaccca |
| | | attgacgtcttttgtcaattggagggcagtgaaaccttgcccttgcttcttgcagaggattcccagtgg |
| | | tctttccgctctcgacaagggaattcatgatccaccaaaagttgtgaagagagcaggtcccatgga |
| | | agctttctgacgactgatgatgactgtagcgaccctttgcaggcagcggacccccccacctggtga |
| | | caggtgcctctgcggccaaaagccacgtgtttaacagacacctgcaaaggcggcacaaccccag |
| | | tgcctcatcaaaagtctgatgactgtggaaatagtcaaccggcttttcttaagcaaatttggtgtcgg |
| | | ggctgaaggatgcccggaaggtaccacactggttgtgatctgatccggggccacagtacatgtgc |
| | | tttacacatgtagctgcggttaaaaaacgtctaggccccccgaaccacggggacgtggttttccttt |
| | | gaaaaccacgattacaat |
| 524 | EMCV1 JZ1203 | gtctgctcgatatcgcaggctgggtccgtgactacccactccccctttcaacgtgaaggctacgata |
| | | gtgccagggcgggtactgccgtaagtgccaccccaaaacaacaacaaaccccccctaacattact |
| | | ggccgacgccgcttggaataaggccggtgtgcgtttgtctatatgttatttcccaccacattgccgtc |
| | | ttttggcaatgtgtgggcccggaaacctggccctgtcttcttgacgagcattcctaggggtctttccc |
| | | ctctcgccaaaggaatgcaaggtctgttgaatgtcgtgaaggaagcagttcctctggaagcttcttg |
| | | aagacaaacaacgtctgtagcggccctttgcaggcagcggaaccccccacctggcgacaggtg |
| | | cctctgcggccaaaagccacgtgtataagatacacctgcaaaggcggcacaaccccagtgccac |
| | | gttgtgagttggatagttgtggaaagagtcaaatggctctcctcaagcgtattcaacaaggggctga |
| | | aggatgcccagaaggtaccccattgtatgggatctgatctggggcctcggtgcacatgctttacatg |
| | | tgtttagtcgaggttaaaaaacgtctaggccccccgaaccacggggacgtggttttcctttgaaaaa |
| | | cacgatgataat |
| 525 | EMCV1 AnrB- | atgtggtcgaagccacttggaataagaccggcgtgcgcttgtctatatgttacttccaccacattgcc |
| | 3741 | gtcttttggcaatgtgagggcccggaacctggccctgtcttcttgacgaacattcctaggggactttc |
| | | ccctctcgccaaaggaatgtaaggtctgttgaatgtcgtgaaggaagcagttcctctggaagcttctt |
| | | gaagacaaacagcgtctgtagcgaccctttgcaggcagcggaaccccccacctggtaacaggtg |
| | | cctctgcggccgaaagccacgtgtataagatacacctgcaaaggcggcacaaccccagtgccac |
| | | gttgtgcgttggatagttgtggaaagagtcaaatggctttccccaagcgtattcaacaaggggctga |
| | | aggatgcccagaaggtaccccactggttgggatctgatctggggcctcggtgcaggtgctttacac |
| | | ctgttgagtcgaggttaaaaaacgtctaggccccccgaaccacggggacgtggttttcctagaaaa |
| | | ccactatgacaat |
| 526 | Cosavirus D1 | cgtgctttacacggtttttgaaccccacaccggctgtttggcgcttgcaggacagtaggtatattttctt |
| | | tcatttctcttttctagccgcgtaggttctatctacgcgggcggagtgatactcccgctccttcttggac |
| | | aggcggcctccacgctctttgtggatcttaaggctgccaagtcactggtgtttgaagtgaagaatgg |
| | | agagacactagggcgtttcatgtggctttgccagggattgtagcgatgctgtgtgtgtgtgcggattt |
| | | cccctcgtggcgacacgagcctcacaggccaaaagccctgtccgaaaggacccacacagtggg |
| | | gttgccccgacccctcccttcaaagctttgtgtaaacaaacttttgtttagactttcttaagcttctctca |
| | | catcaggccccaaagatgtcctgaaggtaccctgtgtatctgaggatgagcaccaccaactaccc |
| | | ggacttgtgggacgtgtcccacagacgcatgtggtattccagccccctccttttgaggagggggct |
| | | tttgctcgctcagcacaggatctgatcaggagattcatctctggtgctttacaccagagcatggattta |
| | | aaaattgcccaaggcctggcaaacaacctaggggactaggttttctctattttaaaagatgtcaat |
| 527 | Cosavirus B1 | cgtgctttacacggtttttgaaccccacaccggctgtttggcgcttgcaggacagcaggtttattttctt |
| | | ttaactctctctttctagccacacacgatctatgtgtgtgggcggagtgatactcccgttccttcttgga |
| | | caggcggcctccacgccctttgtggatcttaaggctaccaagtcactggtgttggaaagtgaagag |
| | | aaaggagttccttgggaactacatgtggcattgacagaggttgtagcgatgctgtgtgtgtgtgcgg |
| | | attacccccgtggcgacacggaccccacaggccaaaagccctgtccgaaaggacccacacagt |
| | | ggagcaaccccagctcccctcttcaatgttttgtgttagcaaccttggtattattttctctcaagcttcca |
| | | atacaccgggccccaaagatgtcctgaaggtaccccgtgtatctgaggatgagcaccatcaacta |
| | | cccggacttgttctttcgagaacagacgcatgtggtaacccagccccgatcctaaggggtcgggg |
| | | cttttgctcactcagcacaggatctgatcaggagacctcccccccctgctttacagggggcggggg |
| | | tttaaaaattgcccaaggcctggcaaataacctaggggactaggttttcctttttattttaaagttgtca |
| | | at |
| 528 | Cosavirus A SH1 | ccgtgctttacacggtttttgaaccccacaccggctgtttggcgcttgcaggacagcaggtttattttc |
| | | ttatgctctttatttctagccaacagggttctatcctgttgggcggagtgatactcccgttccttcttgga |
| | | cagattgcctccacgatctttgtggatctcaaggtgatcaagtcactggtaaatagagcgaaggttg |
| | | aggaaacctgaggaatttccatgtggttttgccaggagttgtagcgatgctgtgtgtgtgtgcggatt |
| | | tcccctcatggcaacatgagcctcacaggccaaaagccctgtccgaaaggacccacacagtgga |
| | | gcaatcccagctccctcctacaaagctttgtgagaatgaactcacgtttattcttctttattctctgtttac |
| | | atcaggccccaaagatgtcctgaaggtaccttgtgtatctgggcatgagcaccatcaactacccgg |
| | | acttgcatttcggtgcagacacatgtggttacccagcccctctgctttggcagaggggcttttgctcg |
| | | ctcagcacgagatctgatcaggagcccttcccagtgtgctttacacctggcggggggttaaaaatt |
| | | gcccaaggcctggcaaaataacctaggggactaggttttccttttattaacaatgtctgtcatt |
| 529 | Malagasivirus B | ctttattttcttatgtaactcttctttttaagttttattttgcctacttgtgagcttatgcgggaccactgtctta |
| | | gacaaccccacatttgtcatgagtaagtacacgcaaccattacgattactttttaaccgtctgacctttt |
| | | gataacaactgaagttaggcgtgaaacatgcatttataccaaagtagccccgcatttccccactacg |
| | | gtgggggggctaccctactggctttggaactgtagccattatgtgttgcctggctttcaggatctcac |
| | | aacacaacagttctctcacaatggaatatgggtgagattgcagtgacatgaacaagtatctagtagt |
| | | acatagactcaagcctagttgcctgcggaacaacatgtggtaacacatgccccagggtccaaaag |
| | | acaagggttaacagccccttctaggtgtctgtgtgtgaagaatactttagtagtgttgttatgatctcac |
| | | ctgttagtacagaatgagtatggcttggtgaaggatgtcctacaggtacccattatatggatctgagt |
| | | aggagaccactagtggtggctttaccgccaggtgagtggtttaaaaagcgtctagccaagccaac |
| | | agcactagggatagtgctttctatattttatattttcagtgtat |
| 530 | Mosavirus A2 | cccccccctcaaattgcaacgatatagctaatggcgagattgagatgctatatcacctcttctaagtt |
| | SZAL6 | atagacctcatctgattgataaggacgtaatttggtcgaaaccgcttggaataagaccgatgcgcgt |
| | | agtcatgatgatgatgtaagatctaggaacttatccaatctgcttatgtctatgtaagtagaggggca |
| | | ggcctcattgccctaattctttctaccgagtatctgctagggtttctagcggcttgaatacttggattga |
| | | gggatacaagatactactgatcgattgtcgattgggaaacttgtagatacttcaaagctaccagtag |
| | | cgtggactcacagccagcggactacccctcatggtaacatgagcctctgggcccacaaggcacg |
| | | tcgcaagacctgtgagacggcaaccccagcctagctttgttgaggaaacaagcgataacatgaca |
| | | tgagagaccggaaggattcttgtattgtgagccgaaggatggcctctaggtacctcattttatgagat |
| | | ctgaggaggtgctcttgagttggtgctttacactgacaacacagagttaaaaagcgtctaagctcac |
| | | ccggaaattgggaaatttccgttatttccattttgtttgcaaagtcgttc |
| 531 | SVV | ctgggccctcatgcccagtccttcctttccccttccggggggtaaaccggctgtgtttgctagaggc |
| | | acagaggagcaacatccaacctgcttttgtggggaacggtgcggctccaattcctgcgtcgccaa |
| | | aggtgttagcgcacccaaacggcgcatctaccaatgctattggtgtggtctgcgagttctagcctac |
| | | tcgtttctcccctatccactcactcacgcacaaaaagtgtgctgtaattacaagatttagccctcgcac |
| | | gagatgtgcgataaccgcaagattgactcaagcgcggaaagcgctgtaaccacatgctgttagtc |
| | | ccttcatggctgcgagatggctatccacctcggatcactgaactggagctcgaccctccttagtaag |
| | | ggaaccgagaggccttcttgcaacaagctccgacacagagtccacgtgattgctaccaccatgag |
| | | tacatggttctcccctctcgacccaggacttctttttgaatatccacggctcgatccagagggtgggg |
| | | catgatccccctagcatagcgagctacagcgggaactgtagctaggccttagcgtgccttggatac |
| | | tgcctgatagggcgacggcctagtcgtgtcggttctataggtagcacatacaaat |
| 532 | PTV A | actagtccttggacttttgttgtgtttaaacacagaaatttaattacctggccatgaattcattggattaa |
| | | cccttctgaaagacttgctctggcgcgagctaaagcgcaattgtcaccaggtattgcaccagtggt |
| | | ggcgacagggtacagaagagcaagtactcctgaccgggcaatgggactgcattgcatatcccta |
| | | ggcacctattgagatttctctggggcccaccggcgtggagttcctgtatgggaatgcaggactgga |
| | | cttgtgctgcctgacagggtcgcggctggccgtctgtactttgtatagtcagttgaaactcacc |
| 533 | PTV B | cttccttttaattcgtaactgataagtgatagtccttggaagctaggtatttgttacgctagttttggatta |
| | | tcttgtgcccaacatttgttttcgaacatatgttgtgtttaaacacagaaatctagtttctttggttatgagt |
| | | ttaatggaatatccttttgaaagacttgccttggcgcgggctagagcgcaattgtcaccaggtattgc |
| | | accaatggtggcgacagggtacagaagagcaagtactcctgactgggtaatgggactgcattgca |
| | | tatccctaggcatctattgagatttctctggagcccaccagcatggagttcctgtatgggaatgcagg |
| | | actggacttgtgctgcctgacagggtcgcggctggccgtctgtactttgtatagtcagttgaaactca |
| | | tt |
| 534 | Tottorivirus | cccctttacgtaactgcaacttaaagagtaccctactgcattggatgtgtggtaaacttttacgcacac |
| | | atttgtagtagtgttagttatgttctacctaatgagtatgcatgcacccgtcgaaacacgcttgtgataa |
| | | gataggtgagtccatgtgactaatctcattaagataaataagcaccctacaacgcacggcacgctc |
| | | gtgtcttccgtgcggggccgggacaacagcggcctaaatcttctaggtgaccaccatgcttttggg |
| | | actatggcaccactgtggacgtgagtacctggcagtaagtctgtgaaaagatggaaggtgtccca |
| | | agctatggggcgtatgcatatagcctgcggaacaaacaacggcgacgttgtccccagggcccaa |
| | | aaggcacgtggataagatccacctatatgtttaccccatagtgtaagtcactggaagtcctagtaatg |
| | | gatgtctggagtaaggctcacggggtagggcgaaggatgcccagaaggtacccgtaggtaacct |
| | | taagagactatggatctgatctggggaccggatggcgccatcaccatgacgtggaggccggttta |
| | | aaaaacgtctaagcccgaccaacaacctaggggactaggttttccttttttattcatgtatgacgtt |
| 535 | Posavirus 1 | acatttccttgcgtgcgcacccgaaaatttattgaacttggcttgaatcataagtaatgcttcttatagc |
| | | ggacactttgagaatataatgcttgtatggattaatgtatactgttttaaataacaaacctagacacgc |
| | | agttgcgttgatggttgtatcaatcacatataagtgttgaactcgtgttaatcctcgatcgctatatgttt |
| | | gccgcctacttccaataaaatagattacatgcgcgtcatgcctttgtgggttacctattggcctctgac |
| | | aaaaacaagtcgtaagatttgtagcttcccggtgtaaaaagctgggcgcggtctggctctcgtagg |
| | | gtggaaaggtccaccaatggctggttgagtgtaagctccggtgtcctggttgtcgcaattccaggc |
| | | gtcgtaataacctatattgcatctgactctaactcttgtggctctactgtatctagttcttgttctactaact |
| | | ctaataatactactggctctaatactgaaaaacttacatatgttaatatagataatatccttgatcctgat |
| | | atccctcacgtcactgaagttcgccgaaaacgaatttcagatcatatcattgaatctcaaggatgtac |
| | | ttgctctgaacctactataactcctcatgcgttttcattttctactcttggc |
| 536 | A105-675 | ccacccacagcaagaatgccatcatctgtcctcacccccatttctcccctccttcccctgcaaccatt |
| | | acgcttactcgcatgtgcattgagtggtgcacgtgttgaacaaacagctacactcacgtgggggcg |
| | | ggttttcccgcccttcggcctctcgcgaggcccacccttccccttcctcccataactacagtgctttg |
| | | gtaggtaagcatcctgatcccccgcggaagctgctcgcgtggcaactgtggggacccagacagg |
| | | ttatcaaaggcacccggtctttccgcctccaggagtatccctgctagtgaattctagtggggctctgc |
| | | ttggtgccaacctcccccaaatgcgcgctgcgggagtgctcttccccaactcaccctagtatcctct |
| | | catgtgtgtgcttggtcagcatatctgagacgatgttccgctgtcccagaccagtccagcaatggac |
| | | gggccagtgtgcgtagtcgtcttccggcttgtccggcgcatgtttggtgaaccggtggggtaaggt |
| | | tggtgtgcccaacgcccgtactttggtgacaactcaagaccacccaggaatgccagggaggtacc |
| | | ccgcctcacggcgggatctgaccctgggctaattgtctacggtggttcttcttgcttccatttctttctt |
| | | ctgttc |
| 537 | A110-675 | acctttgtgcgcctgttttataccccctcccccaactgtaacttagaagtaacacacaccgatcaaca |
| | | gtcagcgtggcacaccagccacgttttgatcaagcacttctgttaccccggactgagtatcaataga |
| | | ctgctcacgcggttgaaggagaaagcgttcgttatccggccaactacttcgaaaaacctagtaaca |
| | | ccgtggaagttgcagagtgtttcgctcagcactaccccagtgtagatcaggtcgatgagtcaccgc |
| | | attccccacgggcgaccgtggcggtggctgcgttggcggcctgcccatggggaaacccatggg |
| | | acgctctaatacagacatggtgcgaagagtctattgagctagttggtagtcctccggcccctgaatg |
| | | cggctaatcctaactgcggagcacacaccctcaagccagagggcagtgtgtcgtaacgggcaac |
| | | tctgcagcggaaccgactactttgggtgtccgtgtttcattttattcctatactggctgcttatggtgac |
| | | aattgagagatcgttaccatatagctattggattggccatccggtgactaatagagctattatatatcc |
| | | ctttgttgggtttataccacttagcttgaaagaggttaaaacattacaattcattgttaagttgaatacag |
| | | caaa |
| 538 | 18-675 | cccacagcaagaatgccatcatctgtcctcacccccaattttcccttttcttcccctgcaaccattacg |
| | | cttactcgcatgtgcattgagtggtgcatgtgttgaacaaacagctacactcacatgggggcgggtt |
| | | ttcccgccctacggcctctcgcgaggcccaccccttccctccccttataactacagtgctttggtag |
| | | gtaagcatcctgatcccccgcggaagctgctcacgtggcaactgtggggacccagacaggttatc |
| | | aaaggcacccggtctttccgccttcaggagtatccctactagtgaattctagcggggctctgcttggt |
| | | gccaacctcccccaaatgcgcgctgcgggagtgctcttccccaactcaccctagtatcctctcatgt |
| | | gtgtgcttggtcagcatatctgagacgatgttccgctgtcccagaccagtccagtaatggacgggc |
| | | cagtgcgtgtagtcgtcttccggcttgtccggggcatgtttggtgaaccggtggggtaaggttggtg |
| | | tgcccaacgcccgtactttggtgacacctcaagaccacccaggaatgccagggaggtaccccac |
| | | ctcacggtgggatctgaccctgggctaattgtctacggtggttcttcttgcttccacttctttcttctgttc |
| | | acg |
| 539 | A115-675 | acctttgtgcgcctgttttataccccccccaacctcgaaacttagaagtaaagcaaacccgatcaata |
| | | gcaggtgcggcgcaccagtcgcatcttgatcaagcacttctgtaaccccggaccgagtatcaata |
| | | gactgctcacgcggttgaaggagaaaacgttcgttacccggctaactacttcgagaaacccagta |
| | | gcatcatgaaagttgcagagtgtttcgctcagcactacccccgtgtagatcaggccgatgagtcac |
| | | cgcacttccccacgggcgaccgtggcggtggctgcgttggcggcctgcctatggggcaacccat |
| | | aggacgctctaatacggacatggtgcgaagagtctattgagctagttagtagtcctccggcccctg |
| | | aatgcggctaatcctaactgcggagcacatacccttaatccaaagggcagtgtgtcgtaacgggta |
| | | actctgcagcggaaccgactactttgggtgtccgtgtttccttttaatttttactggctgcttatggtgac |
| | | aattgaggaattgttgccatatagctattggattggccatccggtgactaacagagctattgtgttcca |
| | | atttgttggatttaccccgctcacactcacagtcgtaagaacccttcattacgtgttatttctcaactcaa |
| | | gaaa |
| 540 | A73-675 | ttactccattcagcttcttcggaacctgttcggaggaattaaacgggcacccatactccccccaccc |
| | | cccttttgtaactaagtatgtgtgctcgtgatcttgactcccacggaacggaccgatccgttggtgaa |
| | | caaacagctaggtccacatcctcccttcccctgggagggcccccgccctcccacatcctcccccc |
| | | agcctgacgtatcacaggctgtgtgaagcccccgcgaaagctgctcacgtggcaattgtgggtcc |
| | | ccccttcatcaagacaccaggtctttcctccttaaggctagccccggcgtgtgaattcacgttgggc |
| | | aactagtggtgtcactgtgcgctcccaatctcggccgcggagtgctgttccccaagccaaacccct |
| | | ggcccttcactatgtgcctggcaagcatatctgagaaggtgttccgctgtggctgccaacctggtga |
| | | caggtgccccagtgtgcgtaaccttcttccgtctccggacggtagtgattggttaagatttggtgtaa |
| | | ggttcatgtgccaacgccctgtgcgggatgaaacctctactgccctaggaatgccaggcaggtac |
| | | cccacctccgggtgggatctgagcctgggctaattgtctacgggtagtttcatttccaatccttttatgt |
| | | cggagtc |
| 541 | Kobuvirus 16317 | ttactccattcagcttcttcggaacctgttcggaggaattaaacgggcacccatacacccccatccc |
| | | ctttctgcaacttaagtatgtgtgctcgtgatcttgactcccacggaatggatcgatccgctggagaa |
| | | caaactgctagatccacatcctcccttcccctgggaggaccttggtcctcccacatcctccccccag |
| | | cctgacgtaccacaggctgtgtgaagcccccgcgaaagctgctcacgtggcaattgtgggtcccc |
| | | ccttcatcaagacaccaggtctttcctccttaaggctagccccgatgtgtgaattcacattgggcaac |
| | | tagtggtgtcactgtgcgctcccaatctcggccgcggagtgctgttccccaagccaaacccctggc |
| | | ccttcactatgtgcctggcaagcatacctgagaaggtgttccgctgtggctgccagcctggtaaca |
| | | ggtgccccagtgtgcgtaaccttcttccgtcttcggacggtagtgattggttaagatttggtgtaaggt |
| | | ccatgtgccaacgccctgtgcgggatgaaacctctactgccctaggaatgccaggcaggtacccc |
| | | acccccgggtgggatctgagcctgggctaattgtctacgggtagtttcatttccaattcttttatgtcg |
| | | gagtc |
| 542 | Aichivirus | ttactccattcagcttcttcggaacctgttcggaggaattaaacgggcacccatacatccccatcccc |
| | Chshc7 | tttctgtaacttaagtatgtgtgcttgtaatcttgactcccacggaatggatcgatccgctggagaaca |
| | | aactgctagatccacatcctcccttcccctgggaggaccttggtcctcccacatcctccccccagcc |
| | | tgacgtaccacaggctgtgtgaagcccccgcgaaagctgctcacgtggcaattgtgggtcccccc |
| | | ttcatcaagacaccaggtctttcctccttaaggctagccccgatgtgtgaattcacattgggcaacta |
| | | gtggtgtcactgtgcgctcccaatctcggccgcggagtgctgttccccaagccaaacccctggcc |
| | | cttcactatgtgcctggcaagcatatctgagaaggtgttccgctgtggctgccagcctggtaacagg |
| | | tgccccagtgtgcgtaaccttcttccgtctccggacggtagtgattggttaagatttggtgtaaggttc |
| | | atgtgccaacgccctgtgcgggatgaaatctctactgccctaggaatgccaggcaggtaccccac |
| | | cctcgggtgggatctgagcctgggctaattgtctacgggtagtttcatttccaatccttttatgtcgga |
| | | gtc |
| 543 | Aichivirus | actccattcagcttcttcggaacctgttcggaggaattaaacgggcacccatacacccccatcccct |
| | Goiania | tttttgcaacttaagtatgtgtgctcgtaatcttgactcccacggaatggatcgatccgctggagaaca |
| | | aactgctagatccacatcctccctcccccctgggaggacctcggtcctcccacatcctccccccag |
| | | cctgacgtatcacaggctgtgtgaagcccccgcgaaagctgctcacgtggcaattgtgggtcccc |
| | | ccttcatcaagacaccaggtctttcctccttaaggctagtcccgatgtgtgaattcacatcgggcaac |
| | | tagtggtgtcactgtgcgctcccaatctcggccgcggagtgctgttccccaagccaaacccctggc |
| | | ccttcactatgtgcctggcaagcatatctgagaaggcgttccgctgtggctgccagcctggtaaca |
| | | ggtgccccagtgtgcgtaaccttcttccgtccccggacggtagtgattggttaagacttggcgtaag |
| | | gttcatgtgccaacgccctgtgcgggatgaaacctctactgccctaggaatgccaggcaggtacc |
| | | ccaccttcgggtgggatctgagcctgggctaattgtctacgggtagtttcatttctaattctttcatgtc |
| | | ggagtc |
| 544 | Aichivirus | ttactccattcagcttcttcggaacctgttcggaggaattaaacgggcacccatacacccccacccc |
| | ETHP4 | ctttttgcaacttaagtatgtgtgctcgtgatcttgactcccacggaatggatcgatccgctggagaa |
| | | caaactgctagatccacatcctcccttcccttgggaggacctcggtcctcccacatcctccccccag |
| | | cctgacgtaccacaggctgtgtgaagcccccgcgaaagccgctcacgtggcaattgtgggtccc |
| | | cccttcattaagacaccaggtctttcctccttaaggctagtcccgatgtgtgaattcacattgggcaac |
| | | tagtggtgtcactgtgcgctcccaatctcggccgcggagtgctgttccccaagccaaacccctggc |
| | | ccttcactatgtgcctggcaagcatatctgagaaggtgttccgctgtggctgccagcctggtaacag |
| | | gtgccccagtgtgcgtaaccttcttccgtcttcggacggtagtgattggttaagatttggcgtaaggtt |
| | | catgtgccaacgccctgtgcgggatgaaacctctactaccctaggaatgccaggcaggtacccca |
| | | ccctcgggtgggatctgagcctgggctaattgtctacgggtagtttcatttccaattcttctatgtcgg |
| | | agtc |
| 545 | Aichivirus | tactccattcagcttcttcggaacctgttcggaggaattaaacgggcacccatacacccccaccccc |
| | DVI2169 | ttttctgcaacttaagtatgtgtgctcgtaatcttgactcccacggaatggatcgatccgctggagaac |
| | | aaactgctagatccacatcctcccttcccctgggaggaccccggtcctcccacatcctccccccag |
| | | cctgacgtatcacaggctgtgtgaagtccccgcgaaagctgctcacgtggcaattgtgggtccccc |
| | | cttcatcaagacaccaggtctttcctccttaaggctagccccgatgtgtgaattcacattgggcaact |
| | | agtggtgtcactgtgcgctcccaatctcggccgcggagtgctgttccccaagccaaacccctggc |
| | | ccttcactatgtgcctggcaagcatatctgagaaggtgttccgctgtggctgccagcctggtaacag |
| | | gtgccccagtgtgcgtaaccttcttccgtctccggacggtagtgattggttaagatttggtgtaaggtt |
| | | catgtgccaacgccctgtgcgggatgaaacctctactgccctaggaatgccaggcaggtacccca |
| | | ccttcgggtgggatctgagcctgggctaattgtctacgggtagtttcatttccaattcttttatgtcgga |
| | | gtc |
| 546 | Aichivirus | gcttcttcggaacctgttcggaggaattaaacgggcacccatacacccccacccccttttctgcaac |
| | DVI2321 | ttaagtatgtgtgctcgtaatcttgactcccacggaatggatcgatccgctggagaacaaactgcta |
| | | gatccacatcctcccttcccctgggaggaccccggtcctcccacatcctccccccagcctgacgta |
| | | tcacaggctgtgtgaagtccccgcgaaagctgctcacgtggcaattgtgggtccccccttcatcaa |
| | | gacaccaggtctttcctccttaaggctagccccgatgtgtgaattcacattgggcaactagtggtgtc |
| | | actgtgcgctcccaatctcggccgcggagtgctgttccccaagccaaacccctggcccttcactat |
| | | gtgcctggcaagcatatctgagaaggtgttccgctgtggctgccagcctggtaacaggtgcccca |
| | | gtgtgcgtaaccttcttccgtctccggacggtagtgattggttaagatttggtgtaaggttcatgtgcc |
| | | aacgccctgtgcgggatgaaacctctactgccctaggaatgccaggcaggtaccccaccttcggg |
| | | tgggatctgagcctgggctaattgtctacgggtagtttcatttccaattcttttatgtcggagtc |
| 547 | Aichivirus rat08 | tactccattcagcttcttcggaacctgttcggaggaattaaacgggcacccactttcctgtcctctccc |
| | | cttttctgtaactccaagtgtgtgctcgtaatcttgactcccgcggattgaccgctccgctggtgaaca |
| | | aactgctaggtcatctcctccccacccttgggcgtccttccgggcgtccacaccctccccccagcc |
| | | tgacgtgtcacaggctgtacaaagaccccgcgaaagctgctaacgtggcaattgtgggtcccccc |
| | | tttgtaaaggaaccgagtctttctcccttaaggctagacccctgtgtgaattcacaggtggcaactag |
| | | tggttccactgcatgctcccgacctcggccgcggagtgctgttccccaagtcgtaacactgaccttc |
| | | acttatgtgcctggcaagcatatctgagaagatgttccgctgtggctgccaaacctggtaacaggtg |
| | | ccccagtgtgcgtagtcttcttccgtcttcggacggtaggtgttaggtaaagatgcggcgtaaggtt |
| | | caagtgccaacgccctggaagggatgacccttctactgccctaggaatgccgcgcaggtacccc |
| | | aggttcgcctgggatctgagcgcgggctaattgtctacgggtagtttcatttccctcttcttccactgg |
| | | catc |
| 548 | Aichivirus Rt386 | actccattcagcttcttcggaacctgttcggaggaattaaacgggcacccactttcctgtcctctccc |
| | | cctttctgcaactcaagtgtgtgctcgtaatcctgactcccacgggttgaccgccccgttggtgaac |
| | | aaacagctaggtcattccctccctacccctgggcgccatttcagtggcgttcatatcctccccccag |
| | | cctgacgtgtcacaggctgtgcaaagtccccgcgaaagctgctcacgtggcaattgtgggtcccc |
| | | cctttgtgaaggaaccgagtctttctcccttaaggctagacccctgtgtgaactcacaggtggcaac |
| | | tagtggttccactgcatgctcccgacctcggccgcggagtgctgttccccaagtcgtgacactgac |
| | | ctccacttatgtgcctggcaagcatatctgagaagatgttccgctgtggctgccaaacctggtaaca |
| | | ggtgccccagtgcgtgtagtcttcttccgtctccggacggtaagtgtgtggtaaagatgcggcgtaa |
| | | ggttcaagtgccaacgccctggaagggatgacccttctactgccctaggaatgccgcgcaggtac |
| | | cccaggttcgcctgggatctgagcgcgggctaattgtctacgggtagtttcatttccctctcttttcact |
| | | ggcatc |
| 549 | Norway Rat | gtataagggttgggaaccttgtaccaagctacctctgccattcagtatttgggagtagaagtagatgt |
| | Pestivirus | gtttacaaactcacacgtgtgggggcggggatagactgtgccagcggtcgtgtaccagcacctac |
| | | gcatacgtgtggactgcgaaccaggagagcacctaggtctgacaagctgtgagaacacagtagt |
| | | cgtcagtgagtcagctggtaaggatcacccacctggatactcacgtggacgagggagtttcccag |
| | | tcagaaacctacaccagaggaggggtcctctggagacatggatggtctgagtaacagactatcta |
| | | ctggggtgtgctgcctgacagggtctcggctgatagcctggctagcagtataaaaatcagttgaatt |
| | | ggcatatgagttgtgaacatctagtaaacaatgaaagacaaaaacaaaaaatgagcataataaaaa |
| | | aattgtacaatccactactcaggtgtggctgcagactt |
| 550 | Porcine | tttgaaaagggggtgggggggcctcggccccctcaccctcttttccggtggccattcgcccgggc |
| | Kobuvirus GS2 | caccgttactccactccactccttcgggactggtttggaggaacacaacagggcttcccatccctgt |
| | | ttaccctttattccatcatcctttccccaagtttaccctatccacaccccactgactgactcctttggattt |
| | | tgacctcagaatgcctatttgacctcccactcgcctctcccttttcggattgccggtggtgcctggcg |
| | | gaaaaagcacaagtgtgttgcaggctaccaaactcctacccgacaaaggtgcgtgtccgcgtgct |
| | | gagtaatgggataggagatgcctacaacaggctcgcccatgagtagagcatggactgcggtgca |
| | | tgtgacttcggtcaccacgggcatagcattgctcacccgtgaatcaagtcatcgagatttctctgacc |
| | | tctgaagtgcactgtggttgcgtggctgggaatccacgcttgaccatgtactgcttgatagagtcgc |
| | | ggctggccgactcatgggttaaagtcagttgacaagacac |
| 551 | Kobuvirus | cctacccaagggttacatgggaccatattcctcctcccctgtaactttaagttttgtgcccgtattcttg |
| | SZAL6 | actccaggcggatgttgtgtcgcccgtcctgtgaacaaacagctagacactttcctcccctccctct |
| | | gggctgctccggcagtccactccctccccccagcgtaacatgccccgctggagtgatgcacctgg |
| | | aagtcgtggacgtgggttagtaacttcggtgaaaacccactataatgacaactggttgacccccac |
| | | actcaaaggactcgagtctttctcccttaaggctagcccggccacatgaatttgcagctggcaacta |
| | | gtgagtccaccatgtcccgcaacctcggctgcggagtgctgttccccaagcgtatgccttccttctg |
| | | taagagtgcgcctggcaagcacatctgagaagtcgttccgctgcgtcgtgccaacctggcgacag |
| | | gtgacccagtgtgcgtagacttcttccggattcgtccggctcttctctaggaaacatgcgtgtaaggt |
| | | tcatgtgccaaagccctgcgcgcggtgttcttctactgccctaggaatgtgccgcaggtacccctac |
| | | ttcggtagggatctgagcggtagctaattgtctacgggtagtttcatttccatcttctcttcaggtcgac |
| | | atc |
| 552 | Kobuvirus sheep | gaccttctggtacttcttcgcctgggtcacaaaagcgaagaacctgcctctctaacgccagacgag |
| | TB3 | cggcattaaacttgaacttctggcactctccactctcccttttccctgtccctttccccactgcgctctc |
| | | aaggtcgcgcaatcctgggactagcccagttttaaaagttcctggcaccctttgcccctctaggccc |
| | | ttaaggtaggaactgaccttgtgctgtgatctcggtgcgggagtgctaccacgtagtcatcgtaagc |
| | | ctcgtttctggttctgccctggcaaggctacagagtaccgtgttccgctgtggatgccatccgggta |
| | | accggacccccagtgtgtgtagcggtatgttcacggtccgccgtgttcaccagattcctgacctgg |
| | | ctttgctagaaatggtgtgtgcccaatccctgtgaccagtatcaattacatcacctaggaatgctagg |
| | | aaggtaccccagtcctgagctgggatctgatcctaggctaattgtctacggtgatgctccttttattttc |
| | | ttacaactgctattgactgtctgattgctgattctgctcttgtgctcttctgctctggctcattctcaaggg |
| | | ttctctttgtccaagatcctttggttctctccttgttccacttgccactgccaacgcttgtc |
| 553 | Pronghorn | gtatacgcagttagttcatcctgtgtatacagattggagactctaaaaacaacgattcggaataggg |
| | antelope | gcccgcggcgaagaccgaagacaggctaaccatgccgttagtagggctagcaccaaaacgcg |
| | pestivirus | ggaactagacacttaggagagtggtctggctactctaagaggtgagtacaccttaaccgtcaagg |
| | | gttctactcctcagttgaggactagagatgccctgtggacgggggcatgcccaagagttagcttag |
| | | ccggggcgggggttgttccggtgaaagtagcaatattgaccacactgcctgatagggcggagca |
| | | ggccccctaggtagtctagtataaaatgtctgctgtacatggcac |
| 554 | Porcine pestivirus | tacgcggggtataacgacagtagttcaagtgtcgttatgcatcattggccataacaaattatctaattt |
| | isolate | ggaatagggacctgcgacctgtacgaaggccgagcgtcggtagccattccgactagtaggacta |
| | Bungowannah | gtacaaataggtcaactggttgagcaggtgagtgtgctgcagcggctaagcggtgagtacaccgt |
| | | attcgtcaacaggtgctactggaaaggatcacccactagcgatgcctgtgtggacgaggacatgtc |
| | | caagccaatgttatcagtagcgggggtcgttactgagaaagctgcccagaatgggtagttgcacat |
| | | acagtctgataggatgccggcggatgccctgtattttgaccagtataaatattatccgttgtaaagcat |
| 555 | Porcine pestivirus | gcagatatcggtggtggacctgggggttgggctcaccgtgccccttcatggggtagacctcactg |
| | 1 | cttgatagagtgccggcggatgcctcaggtaagagtataaaatccgttgttcactaac |
| 556 | Pestivirus giraffe- | gtatacgagtttagctcaatcctcgtatacaatattgggcgtcaccaaatatagatttggcataggca |
| | 1 | acaccccgatgcgaaggccgaaaagggctaaccatgcccttagtaggactagcaaaaaatcggg |
| | | gactagcccaggtggtgagcttcctggatgaccgaagccctgagtacagggcagtcgtcaacagt |
| | | tcaacacgcagaataggtttgcgtcttgatatgctgtgtggacgagggcatgcccacggtacatctt |
| | | aacctatccgggggtcggataggcgaaagtccagtattggactgggagtacagcctgatagggtg |
| | | ttgcagagacccatctgataggctagtataaaaaactctgctgtacatggcac |
| 557 | Classical swine | gtatacgaggttagttcattctcgtatgcatgattggacaaattaaaatttcaatttggatcagggcctc |
| | fever virus | cctccagcgacggccgaactgggctagccatgcccacagtaggactagcaaacggagggacta |
| | | gccgtagtggcgagctccctgggtggtctaagtcctgagtacaggacagtcgtcagtagttcgac |
| | | gtgagcagaagcccacctcgatatgctatgtggacgagggcatgcccaagacacaccttaaccct |
| | | agcgggggtcgctagggtgaaatcacaccacgtgatgggagtacgacctgatagggtgctgcag |
| | | aggcccactattaggctagtataaaaatctctgctgtacatggcac |
| 558 | Human pegivirus | tcagggttggtaggtcgtaaatcccggtcaccttggtagccactataggtgggtcttaagagaaggt |
| | isolate JD2B1I | taagattcctcttgtgcctgcggcgagaccgcgcacggtccacaggtgttggccctaccggtggg |
| | | aataagggcccgacgtcaggctcgtcgttaaaccgagcccgtcacccacctgggcaaacgacgc |
| | | ccacgtacggtccacgtcgcccttca |
| 559 | Human pegivirus | cccggcactgggtgcaagccccagaaaccgacgcctatttaaacagacgttatgaaccggcgcc |
| | isolate GBV-C- | gacccggcgaccggccaaaaggtggtggatgggtgatgccagggttggtaggtcgtaaatcccg |
| | ZJ | gtcatcttggtagccactataggtgggtcttaagggttggtcaaggtccctctggcgcttgtggcga |
| | | gaaagcgcacggtccacaggtgttggccctaccggtgtgaataagggcccgacgtcaggctcgt |
| | | cgttaaaccgagcccattacccacctgggcaaacaacgcccacgtacggtccacgtcgccctaca |
| | | atgtctctcttgaccaataggctttgccggcgagttgacaaggaccagtgggggctgggcgacgg |
| | | gggtcgtataggaagaaaaatgccacccgccctcacccgaaggttcttgggctaccccggctgca |
| | | ggccgccgcggagctggggtagcccaagaaccttcgggtgagggcgggtggcatttttcttccta |
| | | taccgatc |
| 560 | Human pegivirus | tggtcaccttggtagccactataggtgggtcttaagagaaggttaagattcctcttgtgcctgcggcg |
| | isolate JD2B8C | agaccgcgcacggtccacaggtgttggccctaccggtgtgaataagggcccgacgtcaggctcg |
| | | tcgttaaaccgagcccatttcccgcctgggcaaacgacgcccacgtacggtccacgtcgccctttt |
| | | aatgtctctcttgaccaataggttcatccggcgagttgacaaggaccagtgggggccgggggtca |
| | | cagggatggaccctgggccctgcccttcccggcggggtggggaaagcatggggccacccagct |
| | | ccgcggcggcctgcagccggggtagcccaagaaccttcgggtgagggcgggtggcatttttctt |
| | | cctataccgatc |
| 561 | Hepatitis GB | gccgggtggaaggcccggaaccgccccaccacctcaactaggtggtaagggtacgtctatcggt |
| | virus A | ccggctggcccgaaaggcggtggatcctgtgtgttagggttcgtaggtggtaaatcccagcacag |
| | | gtggtaatcgctatagggcaggcttatcccggtgaccgcttccctggatcctggagcgggtcgtgg |
| | | cggcacggtccacaggagtggggcctccggtgtgaataagccctcgtctggagcatcagacgtt |
| | | aaactgagacgtcccgaagagatcggaacgacgccccacgtatggcaacgccgcttaaaaccct |
| | | tcggggacagctatgcgggttgacaatgccagtggggggccgggcccactattgttgtgggctcc |
| | | gagttcctctagggatggccgaaaggcagccatggggccacccaggcggcgccgtgctacagg |
| | | cggcaaggggaaaaatccttcgggtgaccccgggtggcattccctcccttagcagcatgagtgtg |
| | | gtggtagctgcaacc |
| 562 | Simian pegivirus | ggggaatctcaccccccgtccggttccggaagaatcggaaaccgacaccctgaccaatcattctt |
| | | gatcatagagtggatgttagtgaaagccagacgaaagccggcggatgggtggtgacagggttgg |
| | | taggtcgtaaatcccggccaccctggtacccggtataagttgggcggaagctgactgaagctccg |
| | | tgctcttttctgtgcgttcttggtgcacggtccacaggtgacgcctataccggtgtgaataataggcc |
| | | gactcgagcggagtcgttaaactgagaacctccatacggatggcaacttggcttgcgtacgggga |
| | | cgccgctaaagtcacagtgggttaagtccggcgggttgacaaccccagcaaggcgagggggtc |
| | | ctattgttggactctgccagttcccggtggaggtaggcatggggtggcccagctccgcggcgcgc |
| | | tacagccggggtagcccaaaatccgaaaggtgagggcgggccacatgtccgaaatttagtcaag |
| | | c |
| 563 | Pegivirus i | agaatggtctaagtggttgccaccgtggtccgaaggggaggaggacctacgctgccagggttgg |
| | | caggtcgtaaatcccgggtgtaggagatccctccttgttaggactgctggtagctggggggtcggt |
| | | gaccccctgggcaaccgccaaacccggacgaccgggtggcggctccatgttggcacggtccac |
| | | aggtgtgaaccctaccggtgtgaataagggttggtggttgcggtccaccttaaacgtagtatgcatt |
| | | gggcttggtaaaacaccgctcgtagtacggaacgccgcctttaaagacacagtaggcgtagccg |
| | | gcgggttgacaatccatacggggggtggggtgtggtcatggatctgtccacaccaccttcatgcg |
| | | gccctctaagcaagccataccggggggaggcgcgcggcaccgcactgccgggcaaggggaa |
| | | gaaccttcgggtgacccccccccaaccaccgtccgatcaatgctaatgttgcgtttaggcgtgaca |
| | | ccggcaca |
| 564 | Pegivirus K | agaatggtgtgatccgtcgccgctccagcggaaagcgggcgggatctagtggttagggttgttcg |
| | | cgtaaatcccacactagtggtacgctcgtataacgtgggagcagccggTggggtcgacccccc |
| | | Acctggcggctgctgagcaccggacgaagcgcggggggtgaacgctaacccgcggcccggg |
| | | ctgccaacgttaggcacgtcttggctggaagacgttaaacacagggccccccctcaaccctgatc |
| | | cgaggccagagaccaaggtacgccgcccttttaaaggcgttactcgtccaataggatctctccgg |
| | | cgggttgtcaaaccttgctggccctggtgatggttacgggagggggtggggcggggagtagaag |
| | | ccccgcccggcatgggggtaccaagctcggcacgcccagcacgcgtggcgtaggggaaaaat |
| | | ccttcgggtgacccctggtaccataaagtaattaacatgagcatgccgctagggtgtgctttttcttcc |
| | | ttccttgggaaggcggtggcacc |
| 565 | Theiler's disease- | tgataccgtgtcccggtacgacctcgcgcgtccccaagctcgccctgaggggggagcgtaaggg |
| | associated virus | cgcgtagtggggtagccccccaaaccgagccaccctagtgagtgactttagaatggttagggaga |
| | | ctaccgccttcgctgtttggggacctaatgatccgcgtgccagggttcttcgggtaaatcccggcgc |
| | | ggtgttttgggttcagggcagtaggggcagacgggccagcagtcgctggttcctggtaccaccac |
| | | cctatccggacgacctccctcacgaaaggtcgccacggtctgtggctcgacgacgcctataattca |
| | | gtccgaggggcgcagccctcgttaaacttaggcaaggttcctcgccattgatttggccaggggttt |
| | | aagtgaacgccgcccttttaatgtttaatagggttctttcccggcgggttgacaaacacttccctggg |
| | | ctcttcgttggcctcggttccttgatgcttcggcacccatgagcgcacaggggggggaccctgcga |
| | | cagtccgccaagaggaaaatccttcgggtgacctcgtgcgcaacccaatcccttcttcttccacatg |
| | | gcgtgtctgtggtgcatgctgtg |
| 566 | Rodent pegivirus | ggacttcggtccccctgttactctgcgagccaccgcagagccagggttggtacgcccgaggtgtt |
| | | agaccccggccgaaagctcctaaccatggggttagtaggacgtggtaaatgccactgaggggtt |
| | | ggagagctggtagagcgagtaagtcggcgtaaggcccgagtacgggcctcccagcccgggtca |
| | | gcctaaacctggctgtgatacccggtgcatggagggcgtgtcccaacgctcgatcgctgtagggt |
| | | gggtccctgcagttgggtgtggctaccctgctcgtactgcttgatagagtcccggcggacggacc |
| | | agctctcgtcagtccgtggagttgcac |
| 567 | Human pegivirus | aactgttgttgtagcaatgcgcatattgctacttcggtacgcctaattggtaggcgcccggccgacc |
| | 2 | ggccccgcaagggcctagtaggacgtgtgacaatgccatgagggatcatgacactggggtgag |
| | | cggaggcagcaccgaagtcgggtgaactcgactcccagtgcgaccacctggcttggtcgttcatg |
| | | gagggcatgcccacgggaacgctgatcgtgcaaagggatgggtccctgcactggtgccatgcg |
| | | cggcaccactccgtacagcctgatagggtggcggcgggcccccccagtgtgacgtccgtggag |
| | | cgcaac |
| 568 | GB virus | cccccggcactgggtgcaagccccagaaaccgacgcctatctaagtagacgcaatgactcggcg |
| | C/Hepatitis G | ccgactcggcgaccggccaaaaggtggtggatgggtgatgacagggttggtaggtcgtaaatcc |
| | virus | cggtcaccttggtagccactataggtgggtcttaagagaaggttaagattcctcttgtgcctgcggc |
| | | gagaccgcgcacggtccacaggtgttggccctaccggtgggaataagggcccgacgtcaggct |
| | | cgtcgttaaaccgagcccgttacccacctgggcaaacgacgcccacgtacggtccacgtcgccct |
| | | tcaatgtctctcttgaccaataggcgtagccggcgagttgacaaggaccagtgggggccggggg |
| | | cttggagagggactccaagtcccgcccttcccggtgggccgggaaatgc |
| 569 | Equine Pegivirus | agaatggggagttaactcctggcactggcccgaagcatgaactgatcgcggtggcagggttcttc |
| | 1 | gggtaaatcccggccgcgtgttgtgattgtgttagggcaggtgacagtcggcagggtcgaccccc |
| | | tgcttcaggaccactgtcttcctggacgaccgttgctgaaaaagggccgccacggtctgtagctcg |
| | | ccgacgcttctaattcaggccggaggaccacgctccgtaatcgagcccaagtactcaaaccccag |
| | | cacccctgggtcacgccctacgccgcccttttaacgcttcggctaatagggtctatccggcgggtt |
| | | gacaaagggcgcagggttacctggtactacgagcttgggtgtccctgggagtaatcccagggtgc |
| | | c |
| 570 | Culex theileri | atataaatcccagtttggttaaacctatttcaaggcttaagttgtttattattttatcgccgctcgtgactat |
| | flavivirus | aaagttgcctagcggagagagataaagaagaaggagttcaaggctcagggcagggcgcaagtt |
| | | ccctggtccctaggccgctcgcaggaaggaggagtgaagaagaagaaagagaaggagaggac |
| | | caccgccgaaagaaggcaggtgcctcacaagagggccaaccagcgtgttggaccagtggcca |
| | | acgccggacggcgtggtggcctgctgggacgcctggggattggatggagtgccttcctacagga |
| | | agacatcgttcaagccatc |
| 571 | Bussuquara virus | agtatttcttctgcgtgagaccattgcgacagttcgtaccggtgagttttgacttaacgcagtgagaa |
| | | aagttttcgaggaaagacgagaagcgaattctctga |
| 572 | Zika Virus | agttgttgatctgtgtgagtcagactgcgacagttcgagtctgaagcgagagctaacaacagtatca |
| | | acaggtttaatttggatttggaaacgagagtttctggtc |
| 573 | Yokose virus | agtaaattttgcgtgctagtcgctgagcgtcagaccgcaaagtgagtttttagtgatctaaagtgagg |
| | | agttattcttactgtcatcaaacactacaaataaacacgttgaaattatttccggaagaacaactgtcc |
| | | ggaatcaaagacg |
| 574 | Wesselsbron | agtatattctgcgtgctaatcgttcgacgttagtccgtggagtgagcttctattagagtcgttaacacg |
| | virus | tttgaataatttctactgaaaggagtagaagaaaggagattcattccca |
| 575 | Equine | acctccgtgctatgcacggtgcgttgtcagcgttttgcgcttgcatgcgctacacgcgtcgtccaac |
| | hepacivirus | gcggagggattcttccacattaccatgtgtcactccccctatggagggttccaccccgcccacacg |
| | | gaaataggttaaccatacctatagtacgggtgagcgggtcctcctagggcccccccggcaggtcg |
| | | agggagctgaaattcgtgaatccgtgagtacacggaaatcgcggcttgaacgtcatacgtgacctt |
| | | cggagccgaaatttgggcgtgccccacgaaggaaggcgggggcggtgttgggccgccgcccc |
| | | ctttatcccacggtctgataggatgcttgcgagggcacctgccggtctcgtagaccataggac |
| 576 | Hepacivirus B | accacaaacactccagtttgttacactccgctaggaatgctcctggagcaccccccctagcagggc |
| | | gtgggggatttcccctgcccgtctgcagaagggtggagccaaccaccttagtatgtaggcggcgg |
| | | gactcatgacgctcgcgtgatgacaagcgccaagcttgacttggatggccctgatgggcgttcatg |
| | | ggttcggtggtggtggcgctttaggcagcctccacgcccaccacctcccagatagagcggcggc |
| | | actgtagggaagaccggggaccggtcactaccaaggacgcagacctctttttgagtatcacgcct |
| | | ccggaagtagttgggcaagcccacctatatgtgttgggatggttggggttagccatccataccgta |
| | | ctgcctgatagggtccttgcgaggggatctgggagtctcgtagaccgtagcac |
| 577 | Hepacivirus I | cagggtttcgaccctggcccggatacctatcgccttacgccgaaaggtaacgagtaggagtcggg |
| | | tccccaggcccttaccgccaccaagccaggtggggaggtatgggagccggggggtgcagctg |
| | | gtagctccatgggggacgccccgtgagcggatgctgcatcgataccgggttagctctctgggaga |
| | | gcggcacttgacaccacgaatccgggaaccggacaatcgccggcgtgggacgcgttgcctccg |
| | | tggccgagcaatttggcatgcccgtggtgaagagtgatggtgggggggggccccccttccagta |
| | | ccgtactgcctgatagggtcttgcctcaagcccagagagtcgaggctgaaaaccgccatc |
| 578 | Hepacivirus J | gccgctcccgaaagggagtccggcgcgtcatcccactccgaggagtggggtggcgtccccgtg |
| | | tgccggggaaccatgaagcctaagggcatccacattttagaatgaacttgaagcttcgtttcgctgg |
| | | ccggaaagtcctgggttcccatggccagggttccgcaggtgggtaaatcccggtggggttccatc |
| | | caggatatacggcaggcgggcgtagtccggcggttcggacgacgtgtgggtcgcctacggtgg |
| | | attgttcacaggatgggcactccggtgtgaataggccccgtcagggtgcgctgacgttaaactcag |
| | | gccttgcctggtgttcggggaggattgcagggccacgccgcctctaagggccgtatggcacagta |
| | | cttcttcgggcgggttgtcaaggccctccaacgcgacaccagtgcctcggcaggcatggggcca |
| | | cccagctcggcgtcccgcacacagacggcgtaggggaaaatcagcaatgtgaccccgggtggc |
| | | attttccttctctctacttccatgcatgatcaaccgcaatc |
| 579 | Hepacivirus K | gggaacaatggtccgtccgcggaacgactctagccatgagtctagtacgagtgcgtgccacccat |
| | | tagcacaaaaaccactgactgagccacacccctcccggaatcctgagtacaggacattcgctcgg |
| | | acgacgcatgagcctccatgccgagaaaattgggtatacccacgggtaaggggtggccacccag |
| | | cgggaatctgggggctggtcactgactatggtacagcctgatagggtgctgccgcagcgtcagtg |
| | | gtatgcggctgttcatggaac |
| 580 | Icavirus | cgaagtttaagctaagcaccctcgggcgttcccggattatgtgatcacatcaatttgatggctggtca |
| | | ccacgcaacgcctggagagatactcttacttttctcttaagatccccggtcatttgacgcttgtaggat |
| | | gatagggttattttccactataaatactttcatactcttggatgttctatatccaagacgggaggaccta |
| | | ccccgtaccccttagaggtgagatgccaagaacaggccctttctgttctctcgacaatggcatcata |
| | | ggcaacaagcatcacaccaagattgctaagttttgttaagagttcttcaagctatagggtggctgtag |
| | | cgaccttctgatgcctgcggatttccccacggagcgatccgtgccacaggggccaaaagccacg |
| | | gctaacgcccatcaggagcggcacttaccccgtgccccacccttgaaacttgaatgttcacactgg |
| | | cttctctcggctttctgaactgtctgcttgttggggccccgaaggatgccctggaggtaccccatttta |
| | | tgggatctgaccaggggacacctcagctctctaagttgctggtgtttaaaaaacgtctaagggccc |
| | | ccaccccttaggtggagggatccacctttcctttattttttaaaactcttttatggtcacaattgttt |
| 581 | Antarctic penguin | ctaggagactacgcagtgggataagatgactatgatgtcgtacgggcagaagccagtacagtcga |
| | virus A | agtcgagaccgacgtcgaggatttgactctgcctgacctagtgccatc |
| 582 | Forest pouched | tattggatccgcctccgggcaaaggttactttcttgtacctcggcttagccacagggtgaccccttgt |
| | giant rat | acgtaggggccccgacgtagcactggtctgacaacaccttctcggcatttcaccttctgcccgctct |
| | arterivirus | tccgggcggtggtgtcaagaagcagcagtgctcttctcttttcttcctgcagttcaccgagccctacg |
| | | gggggtaggtg |
| 583 | Avisivirus Pf- | cattcccctttccccagccatgggttaaatggcccctcaccaggttcggtgctgtctaggcttccagt |
| | CHK1 | aaagaagtcaaccgagcattgaacaaaacctcagtgggtatggtagttaaccccgtccactggac |
| | | aactttgtcctcttaaaagtggatcaatccaccccaactccccccctagccacctgagccatggtgg |
| | | atagcagtgacgaaactagggaccccaatacctctagtgccaagagaattcccccctcgcgagag |
| | | gtgctcttgggcccgaaaggctagttggcagggtgaagtgaaggaagctgctagcgtggcaacc |
| | | ttaagcgtagcccgaagctgaccttagaggttaaccctagtggaccactggatgaagctgtggag |
| | | gtggtggataggaaagttggccacttgtgagtagatgcccagaaggcataaggctgatctggggc |
| | | cagtgactataccgttccggtaaacctggtataaaaaccatgaaagcaagtgggtttaaaatttcttct |
| | | aattccttcatttcagtagtgataactggcaga |
| 584 | Avian | accaaacaaggactagataacccacgtgaccgttaactggaaaataagatgttgtaggggcgacc |
| | paramyxovirus | tagttggaattcgaccccggctccgaaacctctaattgtggttattggcagtctagtctacttctaacg |
| | penguin | |
| 585 | Newcastle | accaaacagagaatctgtgaggtacgataaaaggcgaagaagcaatcgagatcgtacgggtaga |
| | disease virus | aggtgtgaaccccgagcgcgaggccgaagctcgaacctgagggaaccttctaccgat |
| 586 | Bat Hp- | ttaagcttcggcttgttgcataggaccggaaaggtactatctaccctaactcttgtagttagactctcta |
| | betacoronavirus | aacgaactttaaaactggttgtgtccttcagtagtctgtatggccattggaggcacaccggtaattatc |
| | | aaatactaagaagattcatagtacatccttgtctagcttttggttggcagtgagcctacggtttcgtcc |
| | | gtgtcgctcacaattatccacacagtaggtttcgtccgctgtggttgagttgctagtccgttgctgtttc |
| | | gtcagccatctacaactcgacacc |
| 587 | Basella alba | ggaatatggctaatcggcttattctaatcaaacgcaaaagacttatgacacagaccggacctgaac |
| | endornavirus | gaggtgataaaacacctcgttcaggttcaaaacgtagaagattcattcctccgattagaaatacaact |
| | | acgtctaagcacgatagagatggtatcaagatcggttttagaccacgagaaaatcggcaaatgaaa |
| | | gtacaagttgggtggtttaaattaccaagaacagtaacattcaagaacaacggcaacccgtttgtta |
| | | cctcatttcgtaaattgtttagaagtaacaaagataaattatttaatggtgggaagaacctaagtacag |
| | | taccagccagaagtagtgaaatgacagaaatgtttatgttcatgtccacgctagagggccaattgtc |
| | | aatccaagatcgagatccaaaaataatcaataagtctatatacatgatagaggta |
| 588 | Ball python | ccccttcacccataggcactaggagaacaggataacccctaacggggcatcctgcctgtgaccttt |
| | nidovirus | cagattcgctagttagatatcttcacagactctgctaggcttctgacccagtccgttcccaaagtccgt |
| | | taccgcccgagtagcgcttaggcgcgaaagggacggaagtacctccagtaagcgaaagctgaa |
| | | gtaagggaaatacggcaagactaacttgttagtcttacagtgtggataacctggtagttatccccga |
| | | caagagacctgactcgatgtgaaaacatccaactaggttggcttcaaactagctacaggcaggata |
| | | tcccccaacggggcctccaaggaatcgagaaccaaccctcattccacgtctgtagtaagcaaaaa |
| | | caggggcgatcttcaccgacacctctcaccacagagcacaccaacctctgtgaagccaatttcctc |
| | | gtccaaggacaggttattgagggtcaactttcttccgaccagaagaagggatttcctaccaaaaga |
| | | aaaaccaaatccaccaacaccacaaggtaaaacaacaacttgtgaagccaatacttagtcaaaga |
| | | ctaactattgagggtcaactttctcttcaatagagaagggatttcctggtaaaacaaataacaacaac |
| | | taacatcagcaact |
| 589 | Bat sapelovirus | gacaggtgttttggagggcggatgacgatatctggctggccaccagggaataacggcaaatgtct |
| | | gatcatacggttcacaagtctaccggcgatagtggttcaacaccatgtgtagcagggattcttgcgt |
| | | atgtgaaggcgacagtgc |
| 590 | Bat Picornavirus | taagcggaaagcattcttgtcccccggtcagtaacctataggctgttcccacggctgaaagggtga |
| | 3 | acatccgttacccgcctcagtacttcgagaaacctagtacgcctgatgattccaaattggtatgatcc |
| | | ggtcaaccccagaccagaaactgtggatgggggtcaccattcctagtatggcaacatacaggtgt |
| | | ccccgcgtgtgtcacaggcccttacgggtgccatttcggatgagtctggccgaagagtctattgag |
| | | ctactgttgatacctccggccccctgaatgcggctaatctcaaccccggagccactgggtggtgaa |
| | | ccaaccacttggtggtcgtaatgagcaattctgggacggaaccgactactttggggtgtccgtgttt |
| | | cttttgttcatattaaactgttttatggtcacaacacaacttggtacgatttgtgattattcactgctcactt |
| | | gtcacagtaaatatacacaatcatc |
| 591 | Bat Picornavirus | gggttttacgaaacccgtatacaccagaccttttctcccctccccctccacctaccttttccccctcttt |
| | 2 | ggaccgaaacaaggacacgtaagtggaaacgcgattttatatgtggttggccaccacggaataac |
| | | ggcaattgtctacatgtgggaagtgcaacctccctagccgataacccctgaccgggtgtgtaggat |
| | | aggaaaggtgcccactgtgggcgacaggttatggtagagtggatacctagccaggggcaatggg |
| | | actgctttgcatatccctaatgaagtattgagatttctctgctcattacccggtgatggttgtgtggggg |
| | | gggccccatacactagatccatactgcctgatagggtcgcggctggccgaccataacctgtatagt |
| | | cagttgaattcagccaag |
| 592 | Bat Picornavirus | gaaacccgtatacaccggaccttttctcccctccctctccacttacctttttcccctcttcggcatgaaa |
| | 1 | caaggattattcaagtggaaacgcgatttaatatgcggctggccaccgcggaataacggcaattgt |
| | | gtatctgctggaagccaagcctgcctagccgatagcccttgaccgggtgtgtaggatagcccagg |
| | | aaccagcaatacgcgacaggttatggtagagtagatacctagccaggggcaatgggactgcattg |
| | | catatccctaatgaaccattgagatttctctggtcattacccggtgatggttactagaggggggcctc |
| | | tagtactagatctatactgcctgatagggtcgcggctggccgaccatgacctgtatagtcagttgatt |
| | | tgagcaat |
| 593 | Bat Iflavirus | acgaatcggtatacgcttcggtacctattgttgcaagttcgttccctattttcgatttgcctgcccgaatt |
| | | tgactcaaacaattgtgacatactatgtctctgtttgaaagcactacacgagtttgcgcccagcatgta |
| | | tgttttcaagtcttttgtataagtctgcctctatagtggttttctttgaccttaaagccttgtcaaccatcct |
| | | atatgctgcatcgagacttgatgtcaatctgcctctactacgcaaatgtctagtaattagttataaggtt |
| | | ttactattttccctcatttccaattttagtttgtagtgtatgtgagtatcattcttactccgactgttaagaga |
| | | aaccaatttatagtcgttaaatatgataaatggaatgaatgatggtgtcattttaaaaacactcttctcta |
| | | taggcgtaagcattctcgctcttagagtcgtaaagaagaaatgccgtgtctatcagtatgttatgcga |
| | | tttattttctgccacgcgcttctagtgcaatctagttgacatacagacattgcctaccactcgcgaggg |
| | | tcgaccggtagtgtaaggagtaagtgatgatttccgcttattctgtaccctttgcctggtgaggacag |
| | | atcctgactaattttaaatataaatgaacactagcttccaag |
| 594 | Bat dicibavirus | gtatagcaccggaatggtattttactactccaagtatacgtactaggagttaaaccctgtaatttacag |
| | | gggatttagtgacttttatccgtaaaagtcgattggacgttaatcggtaacgaggccaagtaccgtg |
| | | aaccaatttaaaaacgtattttctcatgtggtagaaccaacttggaaatagcatggcatataggttgttt |
| | | aggg |
| 595 | Betacoronavirus | gataaagtgtgaatcgcttccgtagcatcgcaccctcgatctcttgttagatctaatctaatctaaactt |
| | HKU24 | tataaaaacactaggtccctgctagcctatgcctgagggtttaggcgttgcatactagtgtcttagga |
| | | atttgactgataacacttccctgctaacggcgtgttgcactctcagtctaagcctcccacccatagga |
| | | ggtatc |
| 596 | Betacoronavirus | atttaagtgaatagcttggctatctcacttcccctcgttctcttgcagaactttgattttaacgaacttaaa |
| | England 1 | taaaagccctgttgtttagcgtattgttgcacttgtctggtgggattgtggcattaatttgcctgctcatc |
| | | taggcagtggacatatgctcaacactgggtataattctaattgaatactatttttcagttagagcgtcgt |
| | | gtctcttgtacgtctcggtcacaatacacggtttcgtccggtgcgtggcaattcggggcacatc |
| 597 | Boone | tacgatcgctgtacattccactactgccaattagctcccccttcccgttgctcccctctataaggaga |
| | cardiovirus 1 | gccttctcttgcaaaggtgaagccttcacccccggtcgaagccgcttggaataagacagggttattt |
| | | tctcctctcctcggcgcttgcctcttctaagctgaataggttctatctattcaggcggatggtctggtcc |
| | | gttccttcttggacagagtgtgtatctgggttttccggatctcgaccacacactcaccagagctcagg |
| | | agtgattaagtcaaggcccgatctgcggcgaaaaggaaatgaagtattttgcagctgtagcgacct |
| | | ctcaaggccagcggatttccccacctggtgacaggtgcctctggggccaaaagccacgtgttaat |
| | | agcacccttgagagcggtggtaccccaccaccctgcaaattatggatttgacttagtaactaaaaga |
| | | ttgacttggcatacctcaacctgagcggcggctaaggatgccctgaaggtacccgtgttgaaatcg |
| | | cttcggcgaccatggatctgatcaggggccctgcctggagtggttctatcccacacagcgtagggt |
| | | taaaaaacgtctaaccgccccacaaagaccccggcagggatgccggtttcctttttaccaattcttg |
| | | acact |
| 598 | Breda virus | atcacctagtacttacaagcgggtcaaaccgccctccggaacggtcataaccccctcccgaacgt |
| | | gcgcttgacgtgactggtctttcagtctagctttctgagaaatactccggggttgtaacccaccatttt |
| | | gacctttggtcagtttggtaacactccaaccaaacagcatctgacccacctccagcttgctgcaggc |
| | | catttggacCaaacgggttcagatatcttgtggctaaacctctgacccacctccagtttactgcagg |
| | | ccttttggactaaacgggtAcagactctttgtggttagtttttaactacccactgtttagccgccaacc |
| | | tgatttttattgttacaaaattttgtgtttacacattattttcttacggttggcagtttgtttggttgtttgcaca |
| | | gtttttgctgataccaatttttactgtgcttttggtgttttcggctaaggctgtttttcacatacttagtttgct |
| | | tgaagtaaccttcacaacatctgttttgtttttggtttctaaggtaaagagtttcaggaaaaaacatagg |
| | | cgcccatcttgtggtgtctagttttaattaatctggcaaacaagtatcaagtcatcgactccctttggag |
| | | tgagacttacgagtaccaattcgcctattttggccatccatataaaa |
| 599 | Bovine viral | gtatacgcccagttagttcaggtggacgtgtacgattgggtatcccaaattaataatttggtttaggga |
| | diarrhea virus 3 | ctaaatcccctggcgaaggccgaaacaggttaaccatacctttagtaggacgagcataatggggg |
| | | actagtggtggcagtgagctccctggatcaccgaagccccgagtacggggtagtcgtcaatggtt |
| | | cgacgcatcaaggaatgcctcgagatgccatgtggacgagggcgtgcccacggtgtatcttaacc |
| | | caggcgggggccgcttgggtgaaatagggttgttatacaagcctttgggagtacagcctgatagg |
| | | gtgttgcagagacctgctacaccactagtataaaaactctgctgtacatggcac |
| 600 | Bovine rhinitis A | ttttgcggctctgccgccgttcgggttttacctgttttcacagagcaaaacaggacctctagtttcgtg |
| | virus | cttaaacgagatcatgctcgaactagaactataacgctggtcactggacccgtgccgcgccttgcg |
| | | gatctttgcgggaatggtggctagtgggctgtggaagtgactctaaccacacgcccctcaagtgtg |
| | | ggaaaacacgaactggtgtagcgacgacgataggccttgggacaccctctccagtgatggagac |
| | | ccaaggggccaaaagccacgccttgtgccctgtcgttcacaaccccagtgcagttcgtgccagta |
| | | cctgcttttgggaagtgtgctttggacagctgaaaacagtcctagtgggagactaaggatgcccag |
| | | gaggtacccggaggtaacaagtgacactctggatctgacttggggagagcgggtctgctttacag |
| | | acgccactctttaaaaaacttctatgtctcgtcaggcaccggaggccgggccttttcctttaaaacaa |
| | | tacacttt |
| 601 | Bovine | ttggatctgagcaggggccccctttgggttgctttacaactcaactgggggttaaaaaacgtctaac |
| | picornavirus | ccgacacgccagagggatctggtttccttttatttcttttactcaccactggatgcagattgacgataa |
| | isolate TCH6 | acgttgttgtttgtgactattgacttgatctgcttctacgggtttactttcactgttatacttcttgctttgttt |
| | | ggtgttcactgtactttgtctccttctacatttcaca |
| 602 | Bovine nidovirus | caccaatagattagtcaagctgtctataggcataaactaacccccaaccccattaccccggggcca |
| | TCH5 | ggtgggccgccgccttcgggcaaacccgtgcgctggtataatcaaggttcacagccagattcact |
| | | gccggttagctagtggggcggtagcctggcaaaacccgaagaggttggaaagggaacttcagg |
| | | gtagtttatcctaggctagcgtagctacagttcggtcaagataaccgtcctggtgctagggctagta |
| | | gagacagtggtaacttggacaagggtccagggccactttagggaataccctacggaaggctagg |
| | | tccgtaaggaagacccccgcagttgtccgcggttgagcagagctcctgcgtagacaaaaggcaa |
| | | aaagtggattacattcgcctgcaggaaaaggcaaacgtcgttggagtcggagctaaagtactgga |
| | | cgattgataccacgcctgctgcggtagataaa |
| 603 | Bovine | ccatcgacactccaggctcacggattaagttaggttccgccgaagcgggctaaccaggcccctag |
| | hepacivirus | taggaggcgcctatcccgtgagccctttccccacggattgagtggagctggagctgggaaggac |
| | | cgagtacggtccaatcgagaagaaccctgatgaacattccaggcctcttcggtagatttggatatat |
| | | ccaccagtgaaggcggggtcgtgggtacaggccccctagtccacacagcctgatagggtcctgc |
| | | cgcaggatccgtgggtgcggctgtacatgtacc |
| 604 | Botrytis cinerea | gccccgccgaccttcctatttatttctaaataggaaggtcccgactagtcggataattcggtttaacg |
| | mitovirus 4 RdRp | aattatggttagatctattaaagttaaaatagattgaatttctttctcatcctccttattcctatactttggga |
| | | gtaaatgacaaatgtctatcctcaaaccgaaatggcttaagtgatgaatttgaaagaaaggtaggttt |
| | | taagaatataaggcatcaatatattatacccttgaatgttaagtgaccacggcgtgacgattagggct |
| | | atcttaggatagacagccatctaacgcgacagcagtggaaatcagcttagcatctcaagatcatgta |
| | | taatatatacataaccttacaattataaaaccaaaccaaaacacactataattttatataaattatagaa |
| | | gtatcggacctggacggtacctactattaactgatagtagccaaatgcaggaagctc |
| 605 | Botrytis cinerea | ggaacttttcagttccagaagtggctttattaagccttcaaagtttacactttgaacctgcgattaattcc |
| | mitovirus 2 RdRp | ccatagtgactcttgttactgtgaattaggaatagttgtagttcaacttctaatgaggtgaacaatataa |
| | | taactcatcttattaaccctatacgtagacaattgtccaaagagacagttggaattctgccaatctgga |
| | | atgtttggtacgcgtagaagataataagagaccctctattcccctgcctcatgactaagtcatggccc |
| | | ggggtgtaatagagatacttttatatattatacaatc |
| 606 | Canine | cgctctttatacaaatctgtcaaccctttgtataactctaagccgaacaattatagctaggctttttattat |
| | picodicistrovirus | ataacattaattaggcattagcgttgtcgccaatctcttggtaatcctaaggatacctttcctgttgacta |
| | strain 209 | agatgaagcgccttcggttaccgatgcccggtgtccacgaagccatcgtggtcggccgcgtcccc |
| | | cacctctcccaacttggactccatgttttcagtaggtgtaatgattagtattattgattctgctcgttcaat |
| | | gtgtttatcttcacgatctgggacccaacacatgcttcactcatgtttaaatgttggttccctcattttga |
| | | agacacccaaaccatagagtgcgagaatgaggatttctacttccattctggtaacagaaatgaattc |
| | | ctgcgtgtgtctcgtaaatggaatctttaagaacttcagataaatcgaacaatacactaatacaagttg |
| | | ttttctaccaacatgttcaatgcggctaatctgaccgtggagctgtgaagcgctcaaacccgagtgtt |
| | | gtatacagtcgtaatgcgtaagtccatgaggaaccgactactgttacctctgttggtgtgtttctccttt |
| | | CCtctcttttattattatttgttattgcaaatactacaactttgatcaac |
| 607 | Canine distemper | accagacaaagttggctaaggatagttaaattattgaatattttattaaaaacttagggtcaatgatcct |
| | virus | accttaaagaacaaggctagggttcagacctaccaat |
| 608 | Canine kobuvirus | tttaagtgttgtgcccaatctcttgactcctgctggaaccaccgaccagtagtgtccaaaatgccagg |
| | | tggaaaatcctcccttcccctctgggcttcatgcccggcatcctccccccagcctgacgtgccaca |
| | | ggctgtgcaaagaccccgcgaaagctgccaaaagtggcaattgtgggtcccccctttgtcaaggc |
| | | gtcgagtctttctcccttaaggctagtcctgtcagtgaactctgtcgggcaactagtgacgccactgc |
| | | atgcctccgacctcggccgcggagtgctgccccccaagtcatgcccctgaccacaagttgtgctg |
| | | tctggcaaacattgtctgtgagaatgttccgctgtggctgccaagcctggtaacaggctgccccagt |
| | | gtgcgtaattctcatccagacttcggtctggcaacttgctgttaagacatggcgtaaggggcgtgtg |
| | | ccaacgccctggaacgagtgtccactctaataccccgaggaatgctacgcaggtacccctggctc |
| | | gccagggatctgagcgtaggctaattgtctaagggtattttcatttcccaccctcttcttcttgttcata |
| 609 | Camel | cttaagtgtcttatctatctatagatagaaaagtcgctttttagactttgtgtctactcttctcaactaaac |
| | alphacoronavirus | gaaatttttgctacggccggcatctctgatgctggagtcgtggcgtaattgaaatttcatttgggttgc |
| | | aacagtttggaaataagtgctgtgcgtcctagtctaagggttctgtgttctgtcacgggattccattct |
| | | acaaacgccttactcgaggttctgtctcgtgtttgtgtggaagcaaagttctgtctttgtggaaaccag |
| | | taactgttccta |
| 610 | Cripavirus | gcaaaatcggtagtacgttaaacgtacgaccaccgatgagactgaaatgacactagttgagattatt |
| | | tcaatatcctagtgttataaagtcaatatttgttggttgatcgtttcgtcaatcgatggcgctgacagcc |
| | | ggaaagacggcaataataaaaaccaagatttagtttttaagttttgattgaattgcaaaagctatcttg |
| | | aatagacaatcaaaatattaagtaaagcaaaagcttcttaaagaagacaatatttaattagttagtaac |
| | | caaacctcatcgtgcccctaagggttaaccggttacgtaaaagcgtagaggtattaaggtcactgc |
| | | ggagacctaaaatccgcaattttatgttttgtaatgttttagttatagacttagatgtaactataagagttt |
| | | ataaatacttgtttcaagatttatagacaagatctgatcctatggattttagataaccttcatgttagtgg |
| | | atagtgtgtgtacctatctaaacgcataaggctcttatttcatatttaaagtaggactatgtattacggc |
| | | gcatctaacggtaacgttagtcaagaccggagaatctcggaatgaattttagtaattcccaaatttata |
| 611 | Human | acctttgtgcgcctgttttatatccccaccccgagtaaacgttagaagttacgcaaccccgatcaata |
| | coxsackievirus | gtaggtgtagcactccagctgcatcgagatcaagcacttctgtctccccggaccgagtatcaatag |
| | A2 | actgctaacgcggttgaaggagaaaacgttcgttacccggccaattacttcgagaagcccagtagt |
| | | gccgtgaaagttgcggagtgtttcgctcagcacttcccccgtgtagatcaggctgatgagtcaccg |
| | | cgatccccacaggtgactgtggcggtggctgcgttggcggcctgcctatggggcaacccatagg |
| | | acgctctaatacagacatggtgcgaagagcctattgagctaattggtagtcctccggcccctgaatg |
| | | cggctaatcctaactgcggagcacatgccctcaaaccagggggtggtgtgtcgtaacgggtaact |
| | | ctgcagcggaaccgactactttgggtgtccgtgtttctttttattcttataatggctgcttatggtgacaa |
| | | ttaaagaattgttaccatatagctattggattggccatccggtgactaacaaatcgctcatataccagt |
| | | ttgttggttttgttcccttatcacatacagctcataacaccctcttatatttactacaattgaatagcaaga |
| | | a |
| 612 | Coronavirus | agaaacaagtagtgttttaaaaaccttcaaattagtgcctgtaacatctttgcaatgaaagtagcgctc |
| | AcCoV-JC34 | actagcctctatgcaaagaatgttaaaagaaatacgaagcatttaaagaatacaatctatctaggata |
| | | ggtacaattctcctccccctcttgacttcggtcaactcaactcaactaaacgaaatcccccttgcatg |
| | | gttccgacccgtgtaaggttgtgtatttcgtgcagtcgttgcccttactagtgtaagcgtaacggcatc |
| | | taggtttgcacgtcttggaggaaacggtgtgtacgtttctagtgtttacgccgtatcggttccggccc |
| | | gataggtattgcattagacgtcctgggtggttctgcctgcccttgtgtgattcggctgttccgtcagttt |
| | | ggtcacctcacacgtccttaagac |
| 613 | Chicken | gggtatggtggttaaccccgtccactgggcatctttgccctctcagaagtggatcaatccaccccaa |
| | picornavirus 3 | ctccccccctggttacctgagccacagtggactccggtgacgaagctagggaccccaatacctca |
| | | agtgccaagagagtccccccctcgcgagaggtgctcttgggcccaaaaggctagttggcagagt |
| | | gaagtgaaggaagctgctaacgtggtgaccttaagcgtaattcgaagctgacctttgaggttaacc |
| | | ctagtggaccactggaggaatctgtggaggtggtggttaggaaagttggccacttgtgagtagatg |
| | | cccagaaggcataaggctgatctggggccagtgactataccgttccggtaaacctggtataaaaa |
| | | ccatgaaagcaagtgggtgaaattttctctttttatccttcattcagcagttgatattggcaaa |
| 614 | Chicken | gtggccgacttgcagaacttcctaccgaaccaccacctcacccccataactccttccctctacacct |
| | picornavirus 1 | tccgctatggtggttaccactgctttattgccttgactgagaatggccaccccctcgacacctgcccc |
| | | ctactgccccaccgcgcaaccttttgcttgtccactcggttggagaggcatgggggccccgttcat |
| | | atccccagtccagttggtgaccttccccctccccgtccggtagatggtccagagggctttgccgac |
| | | gccctctatgatgcttgcttgtctttcctccgtcagcgcgagcatgcacttgtcgagcccacggaaa |
| | | cacagcctagcttttgcttctctcaccctgcgtaccctgggcgccttccgctcgagattcgctttgttc |
| | | gacaccctggcgtccccccaccgctacgtgattttactcgtggcatacaccgccctggcgttcagt |
| | | acattccactgcctaatttgggtggcctccctcaatctcccgcaccccccattgcgcacgtcatcac |
| | | cgccgccgctaacgcgatccggcgcggttctcactggcactgtcccctcgtccgccgggtttcca |
| | | ctcatggttggcttttcacttattctggttactggtgttccatcctacttcatgatggtcgccatgaccat |
| | | gac |
| 615 | Chicken orivirus | aaaccctcacgagtgcttgtggtaggtcccaggccaatattcttcgtaaggcttggttccaattttcca |
| | 1 | ccactcgtgtttgggttctggcctatggtacccagaggggcggtttgggggaattaactccccctcc |
| | | cctgtggtcctataccaccccacacctctgtgggctttctttactatcttcttgttttccgacttttaaaca |
| | | ctaggcaggcgcgcctagtcatacaccgcccggctggtctttccagctcttgtgggcggtgcgcg |
| | | ctggtccatcgtgcccagcgacatagcaccttgtggacacctccgaacgccctcccctgtatggg |
| | | gtggtgcccaggggtttcagtgtggtgacacactccctggggcccgaaaggctagtgtgcaacag |
| | | gtgaggtacagccagctgcccccgtggctggagggaccaagcttgtgaagcacacctcaccttct |
| | | tgggggtgggctagtaagtggtgaaagcatagtgtccgtgtcgctggccaacactttgggtcaagt |
| | | ccagccactcagtgagtagatgcccaggaggtacccctagtggatctgacttggggcctgttactt |
| | | aatgcaggttaaaaactatgaaagctgagtagtgtagcccggctggtggcttctcttccttattcattc |
| | | tatttt |
| 616 | Chicken | ggttaacttgtttaaccaaggcttccgtgcagggcttgcacgttaggagttcatgttgattcatgctcc |
| | gallivirus 1 | aatgcccaaaactttgtgtgtttgtttatgtctttcccaaagtttcccccaaggtttcggtactcaaacct |
| | | taattcctagtcccatcttttgggccttagtatctaggaaatgtacccgtgccttgacgaacgtaagaa |
| | | agctgtcttttattgaacggttctaatgaactaagtataactggctcgcgccacctggtgtgtgccgtt |
| | | ggaattcccccatggtaacatggtccaacgggcccgaaaggctagtgggcaatcggtcctccaa |
| | | ggaaggggttcccaccccgacctgaacaggatttgatgaagctcacctcccaggctcctaacccc |
| | | aaggaagtttacttatagtaattagaatttagtatgtaattgctggcaatcttgctagtagtcaggaacg |
| | | ttatgaccaaatgagtagacccccagaaggtaccccattatatgggatctgatctgggcctcatact |
| | | gtgtgtctccccacatatgaggttaaaaccatgaaagtttggtccaaaatattcttttccttttatcttttct |
| | | ttagtggtgacgccattatatcagcagtttgctg |
| 617 | Chicken | ggtgcatcatcactgaacaccctcgggcagagatgcaagggtggaagtcactcctgccccctgg |
| | calicivirus | caacatgcaggtgcccgatcccaagcttagttctgacttcctctcctgggtggtgcaacactccaag |
| | | gttgatgaacaaacctggagggacctctgggcaacctggtctctcgaggatctccggcgcatctcc |
| | | acagactacctcatgctcccggaacctgagaagaacactgatgcctatgatggctggctgatggtc |
| | | ggcgagatgttgacctttgccggtctcattggctgggagg |
| 618 | Carp picornavirus | agctacaggaaagagagagataatcacagcacataaatacaactacagaagagagcctttccctg |
| | 1 | agcactatttacagcaaaccacgctgggaaaagtggtagcatgacccacttacgggttacttagtat |
| | | aggattttaatatcttcgattctttattttcttttcttaactaagttcgcccggactttatccgtgttttatttta |
| | | gtttaagcaaaattgagtaactaagtttttaacctgccaaatggtgagaagtaactctgtgaaaatacc |
| | | atttgtgcatgaaattgtcttgaaaactcttaggcttttggggggtcccactgctgtttggaggactatt |
| | | gacagactctattgtagttgagtagtgactaatgataacgatttgcgtattacgcaatgggctgtacc |
| | | cgttagatttagtatgccggggggaggggtcccactggattgcactatgtaacctgacagggcgtc |
| | | tgccgacgcactacaatgaggataagatcggctgtttttattt |
| 619 | Falcon | cgcttggaataagttgagaggaattatgcatgctagttgtgtttgtttacaactaattgttctaatccaa |
| | picornavirus | gtgaagctcttcgcttggggcggcacgacacttgccgtaattcttctaccgtccctccacaccttgtg |
| | | gatgaagggccggatgtgtggcctctggctaacccctctctctggggtgatgctactggatgtttta |
| | | ctcctagaccaaatcacatgaactcctcttgatccacttcggtggggctatgagcctgcggattaata |
| | | gctggcgacagctaccccaggggccaaaagccacggtgttagcagcaccctcatagtctgatgc |
| | | ccaagggctgatgttgggagctagtagtgtgtgtctggcctatgtttaggacttctggccaagcgca |
| | | gaggagtggggctgaaggatgcccagaaggtacccgtaggtaaccttaagagactatggatctg |
| | | atctggggccccctcacgtggctttaccacgtgttgggggttaaaaaacgtctaggccccaccagc |
| | | ccacgggagtgggctttcccttaaaaagcccaacaatatttatggtgacaattcactgtttcttctttgc |
| | | aatttttgtattcttggactccttattttttgtttgcttgattttagtggacattcagattcaaatacaaa |
| 620 | Equine rhinitis B | cgacaggcacaggtcgctccgagttctagtagtgtgggaacttgttactactgatgaaacgaggta |
| | virus 1 | gtgacactcagtacctgcgaacgaggtcggggccctcccttcttccttcacccaactttcacttttcgt |
| | | tccactttagcaggggtcttctttctatccccctggcggcattggaactagccgtcgcgtcttaacgc |
| | | gcagccctgaaggccccacaccttgtggatcttgccgtgggtatgtttctggcatgtgtttctcaagc |
| | | ctgcaaccgaagccgaacagccacatgaacagtttgagcgtggtagcgctgtgtgagttggcggt |
| | | ggatccccctcgtggtaacacgagcccccgtggccaaaagcccagtgtttacagcacctctcaca |
| | | tccaggacgaccccatcctggcgctcactcttagtagtatggcttagtacgcattaggtggtaagcc |
| | | gagctctccctcggccttgttctgaatgcacacatgtctaggggctaaggatgtcctacaggtaccc |
| | | gcacgtaaccttcagagagtgcggatctgagtaggagaccgtggtgcactgctttacagatgcag |
| | | cccggtttaaaaagcgtctatgcccctacagggtagcggtgggccgcgccctttccttttaaaacta |
| | | cttgttct |
| 621 | Equine rhinitis A | aagggttactgctcgtaatgagagcacatgacattttgccaagatttcctggcaattgtcacgggag |
| | virus | agaggagcccgttctcgggcacttttctctcaaacaatgttggcgcgcctcggcgcgcccccccttt |
| | | ttcagccccctgtcattgactggtcgaaggcgctcgcaataagactggtcgttgcttggcttttctatt |
| | | gtttcaggctttagcgcgcccttgcgcggcgggccgtcaagcccgtgtgctgtacagcaccaggt |
| | | aaccggacagcggcttgctggattttcccggtgccattgctctggatggtgtcaccaagctggcag |
| | | atgcggagtgaaccttacgaagcgacacacctgtggtagcgctgcccagaagggagcggagct |
| | | cccccgccgcgaggcggtcctctctggccaaaagcccagcgttaatagcgccttctgggatgca |
| | | ggaaccccacctgccaggtgtgaagtggactaagtggatctccaatttggcctgttctgaactacac |
| | | catctactgctgtgaagaatgtcctgaaggcaagctggttacagccctgatcaggagccccgctcg |
| | | tgactctcgatcgacgcggggtcaaaaactgtctaagcagcagcagaaacgcgggagcgtttcttt |
| | | ttcctcatttgtttc |
| 622 | Equine arteritis | gctcgaagtgtgtatggtgccatatacggctcaccaccatatacactgcaagaattactattcttgtg |
| | virus | ggcccctctcggtaaatcctagagggctttcctctcgttattgcgagattcgtcgttagataacggca |
| | | agttccctttcttactatcctattttcatcttgtggcttgacgggtcactgccatcgtcgtcgatctctatc |
| | | aactacccttgcgact |
| 623 | Enterovirus sp. | actctggtatcacggtacctttgcacgcctattttataccccttccccatcgtaacttagaagcaacaa |
| | isolate CPML | acaaactgcccaatagcagcacaacacccagttgtgttaggggcaagcacttctgtttccccggaa |
| | | gggtctgacggtatgctgtacccacggcagaagtatgacctaccgttaaccggccatgtacttcga |
| | | gaagcctagtaccattatgaaggttgattgatgttacgctccccagcaaccccagctggtagttctg |
| | | gtcgatgagtctcggcattccccacgggcgaccgtggccgaggctgcgttggcggccagcctac |
| | | accatacggtgtaggacgtcaagatactgacatggtgtgaagagcctattgagctacgtggtagtc |
| | | ctccggcccctgaatgcggctaatcctaactccggagcatccgccagtaagcccactggaagggt |
| | | gtcgtaatgcgaaagtctggagcggaaccgactactttgggtgtccgtgtttcctgttttacttattgtt |
| | | tggctgcttatggtgacaacttatagttatcatcataagctacttggtcttgccaaccggagaattattt |
| | | ggttatttgttggtttcataaacctacagtcgtattttcctgtcttattaattgttctcaaaattaacaaca |
| 624 | Enterovirus | taccgctgcaccagtgagctggtacgctagtaccttcgcacggagtagatggcatcccccacccc |
| | AN12 | gtaacttagaagcaaagtacacatctggccaatagtggcgctgcatccagccgcgcaacggtcaa |
| | | gcacttctgtttccccggtccgcaagggtcgttatccgcccagtccactacggaaagcctactaacc |
| | | attgaagctatcgagaggttgcgctcggccacgaccccggtggtagctctgagtgatggggctcg |
| | | caaacacccccgtggtaacacggatgcttgcccgcgcgtgcactcgggttcagcctattggttgtt |
| | | cacctcaacatagtgtaaatggccaagagcctactgtgctggattggttttcctccggagccgtgaa |
| | | tgctgctaatcccaacctccgagcgtgtgcgcacaatccagtgttgctacgtcgtaacgcgtaagtt |
| | | ggaggcggaacagactactttcggtactccgtgtttcttttgttttattttgaattttatggtgacaattgc |
| | | tgagatttgcgaattagcgactctaccgctgaacattgccctgtactacctaatcgcatttcacaaaa |
| | | cctcagagataccaagctcttacattgatctgcttgttttcctgaatctcaaatataaattggaacaagc |
| | | aaa |
| 625 | Dolphin | accagacaaagctggctaggggtagaataacagataatgataaattatcatacttaggattaatgat |
| | morbillivirus | cctatcaattggcacaggatttggataaaggttcacagtc |
| 626 | Dianke virus | tgttttcaaccataatactactactacaagtataaaaccccgtccgtctgtcggagacgctaaactctg |
| | | accaccaatctagccacatcagttgcttaaagaacctcttgagacactctcccacttaacatcttttag |
| | | gaatcttcgatgctacaacaacttggctagtgaacaataaatccgtacaattcacagttgtaagagg |
| | | ccataggtccagactttgaaaggtttgtttctattgttacaaatacttagattaacagaggctatttaata |
| | | gtgctcatcacgttaacagagtaaccttgtgcaatagtatgagcttgttgtaaaacgtcttgatacgac |
| | | acc |
| 627 | Guereza | cactcaatactacactccgcatttggggagaagcgctggcgttcgcggaaccgcgttaaccatac |
| | hepacivirus | gcgtagtacgagtgcgacagaccccggtgctactggtggtagcgagacacgagccgaagtctgt |
| | | ggggggaactccacttagagggcatgcccgggcgtaggcttctgagttgggatgggccccaact |
| | | tggcccctgagtgggggggtgttacgacctgatagggtgcgggctggcgcctaccactttccagt |
| | | cgtacatgagtc |
| 628 | Grapevine | gcccggggggtgcagtcctgtgaaagggtctgcaccatactatatatgtatatgattacatcccaaa |
| | associated | aggcgacttcgttcaggttttaaatctgacgtaggtccagtaaataagcatgtcaaaacatgtaagttt |
| | narnavirus-1 | atcctgtaatctactctcataagatgagataagatgatattgcagttcccatgtaaataaatccattatg |
| | | aattcattcatataaggtagaagtggtaactatggttgaaacattaatataaaacggtcattttgcatga |
| | | acgtcattaaggaactggcataccaatgtctatttagtgactatgatatttagagtatcccttatattaat |
| | | taacaattattccttttagcatatcatccgacaacaaattttaaaagaagaaatattactcattaaaa |
| 629 | Goat torovirus | gtacttacaagcgggttaaaccgccctccggaacggttacaaccccctcccgaacgtgcgcttga |
| | | cgtgactggttctcagtctggctttctgagaaatactccagggttgtatcccaccatcttgacctctgg |
| | | tcattttggtaacaccataaccaaacaaactctacacacctaacccacctccagcttgctgcaggcc |
| | | ttttggactaaacgggtttaggtgttttgtgaccaactcgtctacccacctccagttttactgcaggcct |
| | | ttttggactaaacggCtttagacttttgtggttagtttttaactacccactgtttagccgccaacctgatt |
| | | ttcattgttgtaaaattttgtgtttatacactattttcatacggttggcagtttgtttggttgtttgcacagttt |
| | | ttgttgataccaatttttactgtgcttttagtgtattctgctaaggctgtattttacatacttagtttggttga |
| | | agcaatttttacaacatttatattgtttttgatttctaaggtaaagagtcttaggaaacaccatagacgcc |
| | | attcttgtggtgtctagttccaactaatctggcaaacaagtaccaagtcattgactcactttggagtga |
| | | gacttacgagtaccaatttgcctatttcggacatccatataaag |
| 630 | Foot-and-mouth | acaagcttgacaccgcctgtcccggcgttaaagggaagtaaccacaagcttacaaccgcctaccc |
| | disease virus O | cggtgttaatgggatgtaaccacaagatacaccttcacccggaagtaaaacggcaaattcacaca |
| | isolate | gttttgcccgtttttcatgagaaacgggacgtctgcgcacgaaacgcgccgtcgcttgaggaggac |
| | | ttgtacaaacacgatctaaacaggtttccccaactgacatacaccgtgcaatttgaaactccgcctg |
| | | gtctttccaggtctagaggggtaacactttgtactgtgcttgactccacgctcggtccactggcgagt |
| | | gttagtaacagcactggtgcttcgtagcggagcatggtggccgtgggaactcctccttggtaacaa |
| | | ggacccacggggccgaaagccacgtcctgacggacccaccatgtgtgcaaccccagcacggc |
| | | aacttttctgtgaaactcactctaaggtgacactgatactggtattcaagtactggtgacaggctaag |
| | | gatgcccttcaggtaccccgaggtaacacgcgacactcgggatctgagaaggggactggggctt |
| | | ctgtaaaagcgcccagtttaaaaagcttctatgcctggataggtgaccggaggccggcgcctttcc |
| | | attataactactgacttt |
| 631 | Feline infectious | acttttaaagtaaagtgagtgtagcgtggctataactcttcttttactttaactagccttgtgctagatttg |
| | peritonitis virus | tcttcggacaccaactcgaactaaacgaaatatttgtctctctatgaaaccatagaagacaagcgttg |
| | | attatttcaccagtttggcaatcactcctaggaacggggttgagagaacggcgcaccagggttccg |
| | | tccctgtttggtaagtcgtctagtattagctgcggcggttccgcccgtcgtagttgggtagaccgggt |
| | | tccgtcctgtgatctccctcgccggccgccaggaga |
| 632 | Farmington virus | acgacgcataagcagagaaacataagagactatgttcatagtcaccctgtattcattattgacttttat |
| | | gacctattattagacccttcacgggtaaatccttctccttgcagttctcgccaagtacctccaaagtca |
| | | gaacg |
| 633 | Avian infectious | acttaagatagatattaatatatatctattacactagccttgcgctagatttttaacttaacaaaacggac |
| | bronchitis virus | ttaaatacctacagctggtcctcataggtgttccattgcagtgcactttagtgccctggatggcacctg |
| | | gccacctgtcaggtttttgttattaaaatcttattgttgctggtatcactgcttgttttgccgtgtctcacttt |
| | | atacatctgttgcttgggctacctagtgtccagcgtcctacgggcgtcgtggctggttcgagtgcga |
| | | ggaacctctggttcatctagcggtaggcgggtgtgtggaagtagcacttcagacgtaccggttctgt |
| | | tgtgtgaaatacggggtcacctccccccacatacctctaagggcttttgagcctagcgttgggctac |
| | | gttctcgcataaggtcggctatacgacgtttgtagggggtagtgccaaacaacccctgaggtgaca |
| | | ggttctggtggtgtttagtgagcagacatacaatagacagtgacaac |
| 634 | Human | ttaaaactgggtgtgggttgttcccacccacaccacccaatgggtgttgtactctgttattccggtaac |
| | rhinovirus 1 | tttgtacgccagtttttccctcccctccccatccttttacgtaacttagaagttttaaatacaagaccaat |
| | | agtaggcaactctccaggttgtctaaggtcaagcacttctgtttccccggttgatgttgatatgctcca |
| | | acagggcaaaaacaacagataccgttatccgcaaagtgcctacacagagcttagtaggattctga |
| | | aagatctttggttggtcgttcagctgcatacccagcagtagaccttgcagatgaggctggacattcc |
| | | ccactggtaacagtggtccagcctgcgtggctgcctgcgcacctctcatgaggtgtgaagccaaa |
| | | gatcggacagggtgtgaagagccgcgtgtgctcactttgagtcctccggcccctgaatgcggcta |
| | | accttaaacctgcagccatggctcataagccaatgagtttatggtcgtaacgagtaattgcgggatg |
| | | ggaccgactactttgggtgtccgtgtttcactttttcctttattaattgcttatggtgacaatatatatattg |
| | | atatatattggcatc |
| 635 | EV22 | ccttataacccgacttgctgagcttctataggaaaaaaccctttcccagccttggggtggctggtcaa |
| | | taaaaacccccatagtaaccaacacctaagacaatttgatcaaccctatgcctggtccccactattc |
| | | gaaggcaacttgcaataagaagagtggaacaaggatgcttaaagcatagtgtaaatgatcttttcta |
| | | acctgtattatgtacagggtggcagatggcgtgccataaatctattagtgggataccacgcttgtgg |
| | | accttatgcccacacagccatcctctagtaagtttgtaaaatgtctggtgagatgtgggaacttattgg |
| | | aaacaacaatttgcttaatagcatcctagtgccagcggaacaacatctggtaacagatgcctctggg |
| | | gccaaaagccaaggtttgacagacccattaggattggtttcaaaacctgaattgttgtggaagatatt |
| | | cagtacctatcaatctggtagtggtgcaaacactagttgtaaggcccacgaaggatgcccagaag |
| | | gtacccgcaggtaacaagagacactgtggatctgatctggggccaactacctctatcaggtgagtt |
| | | agttaaaaaacgtctagtgggccaaacccaggggggatccctggtttccttttattgttaatattgaca |
| | | tt |
| 636 | Human TMEV- | tccgacgtggttggaattaacatcttttccgacgaaagtgctattatgcctccccgattgtgtgatgctt |
| | like cardiovirus | tctgccctgctgggcggagcgtcctcgggttgagaaaccttgaatcttttcctttggagccttggctc |
| | | ccccggtctaagccgcttggaatatgacagggttattttccaaactctttatttctactttcatgggttct |
| | | atccatgaaaagggtatgtgttgccccttccttctttggagaatctgcgcggcggtctttccgtctctc |
| | | aacaggcgtggatgcaacatgccggaaacggtgaagaaaacagttttctgtggaaatttagagtg |
| | | gacatcgaaacagctgtagcgacctcacagtagcagcggattcccctcttggcgacaagagcctc |
| | | tgcggccaaaagccccgtggataagatccactgctgtgagcggtgcaaccccagcaccctggttc |
| | | gatggccattctctatggaaccagaaaatggttttctcaagccctccggtagagaagccaagaatgt |
| | | cctgaaggtaccccgcgcgcgggatctgatcaggagaccaattggcagtgctttacgctgccactt |
| | | tggtttaaaaactgtcacagcttctccaaaccaagtggtcttggttttccaattttgttgactgacaat |
| 637 | Human | acttaagtaccttatctatctacagatagaaaagttgctttttagactttgtgtctacttttctcaactaaac |
| | coronavirus 229E | gaaatttttgctatggccggcatctttgatgctggagtcgtagtgtaattgaaatttcatttgggttgca |
| | | acagtttggaagcaagtgctgtgtgtcctagtctaagggtttcgtgttccgtcacgagattccattcta |
| | | caaacgccttactcgaggttccgtctcgtgtttgtgtggaagcaaagttctgtctttgtggaaaccagt |
| | | aactgttccta |
| 638 | Hubei zhaovirus- | gtgcaggatggcctttcccatcttaagtggtttgtaggatttcgtgggtccataccccccgatttcttg |
| | like virus 1 | gtacgtattccatgcacggagaatacgaccaaaactcttatttcaaaaaatattattttttactcttgtgg |
| | | gctgagtgcgacccaccagttccagcttagcaacctggaagttgttggagatttatggaaccaaatt |
| | | acacatgcgtggagtgccgccactccgtatctgttcactcattacgcgattaagttctgcgacgaga |
| | | cgagcgaa |
| 639 | Hubei tombus- | ggaccatccaggcaggtgtaggctagtaccctcacctgacctgtcgcgatgtttggctttgtgagg |
| | like virus 9 | cttgtgggaggatcccttggcccatgcattgctgctgtcatcgtgaaaaatgttgtatgctcgcacct |
| | | ggcgtggaggaaacggcatttgacggatgctaaggaggatttgaaggtcctcaactcccatgcctt |
| | | attacaagtcccctttccttcaagcttcgagcccacgtctgtgacgagcagaggtgaggttggagtc |
| | | aagaggagtcctattcaagctgttcgcagcaagaaccataaaactttcattgctcaatgggcaaga |
| | | gcggctaaggctcggttctcgtttgcacgacagtgtgagcggagccctgtaaatgtcgcggccctt |
| | | catcggtggtttgcatcggagtttaagaaaattggcatgaacttgctccaggtttcgtacgtgatcgat |
| | | gaggttgttgaacttagtttggaaccaacgtatgagcgtatggtgtccgaaactaagaagcaattcc |
| | | gtcatcgggcacgtatggaatactacaacgagaaaaaatgccttgaaaagatccactaggaacgc |
| 640 | Hubei tombus- | ttcgggtttacccgcgtaagcggccacactgactggttgtcggtgttgaatttgtataccagatgagg |
| | like virus 32 | agacgttaccaccgtctcggcagtgctacgtctgggaaaggactgtgatagtggacagtcctacc |
| | | gtctttagatacattgcgttgtgtatagcccgggagggattaactaatagcaacgcaatgcacacgg |
| | | cggttcggatttgcttgactgatggaaagacatctaagttctaattgaacc |
| 641 | Hubei sobemo- | gagatgtttgcgtgggccgttgcgctgcgggcggcccacctccctacggggaccgtgagacacc |
| | like virus 3 | gctggggaaggcccccacccccggccaaggggatcctgccgagaggcaggagaaagaggcc |
| | | cagccctctggggcgcctttaggggTgcctgggagggaagtacccgagccgggcggccggtc |
| | | gggtgcggctgtgcagttcgaggctaaccgtaaggaaggcctgagctgcctcggcttgtcggaa |
| | | aggaagacgaaggcacttatcaaggctttcaggaagcaggagcgcaattacagcgcgcgccgt |
| | | gcggcctggattaaccgcatttggccg |
| 642 | Hubei picorna- | agcaacttctttctgaaaactagctagagttcgacgatctctctggctaatgacaaataaccaatcaa |
| | like virus 2 | aaagtcaaatgttcatgtatatatatatttagtagtgacctttatttagaaaaactttagatgatttatcgtc |
| | | aagttgccctttgtgaagcgatcagctttttatatcgtttcatttttgtatacgtcttaattgacgttgtaag |
| | | tacgttttgcatacctcacattgaggatagtatcgtttcctgactaagaagttaaactagtctaccaata |
| | | gcaaccatataggatatagctttgtttttaacaaggtttaatctgatcttatgctcttgcttacggtgtttta |
| | | tgtatagaaaaGtttttataaaaactacataattgtcaaaagaaaaagcgttacgtactgacgcattta |
| | | tgttcacagtgtgacacaaaccttctatattttgattgtaaaataggctttgcctgaccatttatcaaata |
| | | caaactgtttcaaacgcctctccgagccatattggccgacgcgaatcgacataacagggtgagttt |
| | | acagctgcagttgcagccgaggatccacttatttgagagagaattactcttaacgaaattttgaagtc |
| | | acttctacacagttaagtctgagtaggcgttatccaaacgtaaa |
| 643 | Hepacivirus P | acatgggggggggctgacagtgagtacactgtgccaagcaggtgctacgctatgcctaggtgct |
| | | gctgtaggccaaggacatgtcccagtcatcccaggtgagggggggggttcccctcaccgctgcc |
| | | actgcctgatagggtcctgccggagggtctcggtgtccggctgtac |
| 644 | Harrier | gatgtgtgacggtgtaattactttccggatcccactttcctattataactctttcatcccaaggttaggg |
| | picornavirus 1 | aaagaacctggctcggtaccaccagaccctccgccacgctagtggactctccggagataacggt |
| | | accccctttgtagtcacctgtgctggtgaagaaccacctagtattgcttgggtgcgtgccgcctagct |
| | | tccatttcttctggagcactgtgcaatgaggtttccccacttggtaacaagtgcctcaggtcccgcaa |
| | | ggatactgtggggtggtgtgaccgcagggagctgtctccacggctcctctaatgttacgccgctat |
| | | ccacaggccagtgcgtgtcatcgatcccggatgacagagctagtattgcgaacccccaagtaaga |
| | | aaagtggctagtaacctgatagctggtgaagagggtgggtcagttgagtagatgccctagaggta |
| | | cccgaaaggatctgactagggacccgtgactatacattaggtaaaccgggtataaaaaccatgaa |
| | | aaactgaccacttttcttttaacctcttctttctttttatgtgtgttaagtgttttgagtttggactgtaccttg |
| | | cccgcctttcttggattttctctatcgctcttcttacacctactgttatcaaggcactctttagagata |
| 645 | Kunsagivirus 1 | tttcaaatcggactccggtagttataccggagcccggtttggacgcttgggccgcgttaacagccc |
| | | cccacccctttcccactgactgatttctcggattggactcatttgcattgctaactctgattctggatttc |
| | | cccgtttatgtcgtcgcggtcggaagtgcacgtacttcgacgttgatctgatggcgtttgtttccagg |
| | | ggggaggtggcggcagaaacgcccccgccgtaaacttcggcgggccacgcctgtcaagccact |
| | | ccctggggccgagcgcctgaggtgatacagagagataagcacactgggcgctgacaacgcccg |
| | | ggacctcagtgagaagagcagtagggccgtgtttatgggactccattggatatcccccgcttgtcg |
| | | gaactcacggctactccgggttgggaagcccgcgactggtactgtactgggtgatagcctggtgc |
| | | cttccctctcactgttgtatgaaggctgaaaaccccct |
| 646 | Kagoshima-2-24- | tatagtttgcctgtttctcgcaccgttaccgctcgttcgggaatgtgaaactggcacccctcctctccc |
| | KoV | ctaccaccctttctccttcgcccccattcataatttacaacgccgcacacagcggcggccgccaag |
| | | ggctagcctggcggttataaaaggaacctgggtctttccctcttcaagccaaaaggtaggttccctg |
| | | tgtccctgaatgctcggtgaggaatgctgcaccgtaacgctttgtgaagtgtttgcaagttctggccc |
| | | ggcaagcctacagagtgctgtgatccgctgcggacgccatcctggtaacaggacccccagtgtg |
| | | cgcaacagtatgttcagacttcggtttgttcacttgctttcatggaccattgcgcgaaagtgcgtgcg |
| | | ccatatccctgtacttcaggtgtgcttctctggaccctaggaatgctgcgaaggtaccccgtttcggc |
| | | gggatctgatcgcaggctaattgtctatgggttcagtttcctttttctttactccacaattgactgcttaa |
| | | ctgactctggatcttgtgcttccactgctctttctgctctcaaaacggcttcacttaccaactctcacctt |
| | | tcgaccaacaccatttacacactaacttttttcgactcttctgactcctggcttggtgaagac |
| 647 | Kashmir bee | tacgtacaattttgacgcttcgttcatgcaacaatgaactcacatgtggcgctcggtagtaaccagag |
| | virus | gggcgtcattcccccgtatggagtggagaatataagctaccgactcgagctgtagaataattcagc |
| | | aacttataacgaacacgaattttagtcgacgaaaccattttagcctttaattctatgatttgattataatg |
| | | ataaacagctcatgtaactgtctaaactacataaatacaactggattacgaacctttaagtaactatctt |
| | | agatgaagtctagtagtctcccaatatttccgtgaaaagaatgagggacgagatagctctatttaaa |
| | | gacgtgaggctttaaattctgataaatacattacctgagaattcctcctttaggagttagaatttgaaaa |
| | | gattagtacctatacttaatagaatttaaatatgttaataatgctgaagacaagtatttcgattattaacct |
| | | ctatatttctatataaagtatctgtgagtctcagtggacatcacagtaaggtcgcagttaacttgtaatc |
| | | ttttcattcctgtgtcggagcagtggtaatggagccggacgatttcgccaaaac |
| 648 | Jingmen picorna- | tgtgtttttgtcaagataattgttctgtgattaacagtgattgtggttcgtgtaatgcgacgcagtcaaat |
| | like virus | gctagttttgatgaagtgtatgagagagtggaaaacttatctcataagaagattgaagagtgtgtaga |
| | | tcaggctattgatcgagcttctaagcttcgtgattacaagcttaatgttcacaatggctcccgacggg |
| | | aatcatctgatcctctctttatttcgccccattcgttgttatcgcttggggtatctaagtttgttgcgtttga |
| | | gcagcatcacagtttcgcttcagttgagtctctgaagttgcttgctctgtct |
| 649 | Mumps virus | accaaggggaaaatgaagatgggatgttggtagaacaaatagtgtaagaaacagtaagcccgga |
| | | agtggtgttttgcgatttcgaggccgggctcgatcctcacctttcattgtcgataggggacattttgac |
| | | actacctggaaa |
| 650 | Mouse Mosavirus | gaagttgatcatgaacttggttattggtggaacgcacatgaactcccaacaatgatcttgaagacac |
| | | agcgtggtaacaattaccatgcccttgtggctgcccaagacattgatggctattgggtgatttatgat |
| | | gac |
| 651 | Miniopterus | gttgtcgacccgttgcttggtttaagcatgaggtggattcccccgattatgtctacccgttactatggc |
| | schreibersii | gggcggtcgtttcagggtgtttctactgaggactgcaccaagtctttatcttttattctcagatctccgg |
| | picornavirus 1 | ctgtttgacgcttgtaggacagcaggactattttctcttaatctttttctacccactagggtcctatccta |
| | | gtggagggagggtgccaccccttctctctttagagagtgcgcctggcggtctttccgtctctggaaa |
| | | aaggagcacatggcatgctacaattggcacaagaaaacaagctttgcggattctttctttgtactaga |
| | | ggaagctgtagcgaccctgtatggcgagcggactcccctctcggcgacgagagcctctcgggcc |
| | | aaaagccaagtgttaatagcacccatacaggcggcagtaccccactgccctttctcaacatacaat |
| | | gactgatgaaccactgttggtttttctgacacctttgtagtaggattccaaggaatgtcctgaaggtac |
| | | cctgttagcttacgcgcaggatctgatcaggagtctctttacagtgctgtacactgtgcaagggatta |
| | | aaaattgtttgaggaatccccgagatagtggtctatctcttcctattttgttttacagacacg |
| 652 | Linda virus | gtatagcagcagtagctcaaggctgctatacgattggacataccaaattccaattggtgttagggac |
| | | cacctaggtgaaggccgacgacaggtagccattcctgttagtaggacgaaccgttatggtggact |
| | | ggttgctcaggtgagcaggctgcaatgcgtaagtggtgagtacaccacagccgtcaaaggtgcc |
| | | actggtaaggatcacccactggcgatgccttgtggacgggggcgtgcccaacgcaatgttagcg |
| | | gtggcgggggctgccatcgtgaaagctaggtcttgatggaccttgttgcctgtacagtctgatagg |
| | | atgccggcggatgccctgtgacagccagtataaagaatatccgttgtgattgcac |
| 653 | Lesavirus 2 | tctttctttattttcttatgtaactcttctttttaagttttattttgcctacttgtgagcttatgcgggaccactg |
| | | tcttagacaaccccacatttgtcatgagtaagtacacgcaaccattacgattactttttaaccgtctga |
| | | ccttttgataacaactgaagttaggcgtgaaacatgcatttataccaaagtagccccgcatttcccca |
| | | ctacggtgggggggctaccctactggctttggaactgtagccattatgtgttgcctggctttcaggat |
| | | ctcacaacacaacagttctctcacaatggaatatgggtgagattgcagtgacatgaacaagtatcta |
| | | gtagtacatagactcaagcctagttgcctgcggaacaacatgtggtaacacatgccccagggtcca |
| | | aaagacaagggttaacagccccttctaggtgtctgtgtgtgaagaatactttagtagtgttgttatgat |
| | | ctcacctgttagtacagaatgagtatggcttggtgaaggatgtcctacaggtacccattatatggatc |
| | | tgagtaggagaccactagtggtggctttaccgccaggtgagtggtttaaaaagcgtctagccaagc |
| | | caacagcactagggatagtgctttctatattttatattttcagtgtatatggtgacaa |
| 654 | Lesavirus 1 | gtaactaataagcaagttttactgcctgcaaactgcttcaatgggaccaccgcttcggcgaccccttt |
| | | gttgagtttgtatgtttttaagtaatctttgcaaccatacgattattttagccgcctctctataatgatcttgt |
| | | tatagtgggacgtgaaacattggtttttctcacacacgtccggtcacccgggcgtgtgttcttccgta |
| | | agtcctatccacataccatcgtgggtaggccagcatgtttgcacaggctgtgacagtgtgggtggg |
| | | ctttccacctctcaacaacacactgaattgcaatgcactcacggaggaaatgacaatttggttatagt |
| | | tttgaactgtgctagtaatttctttcacattaagccatgttgcctgcggaatcacatgtggtaacacatg |
| | | cctcagggcccaaaaggcacgggttagcagccccttcatggtgtgttagaagtgaaaacacatag |
| | | tatgagctataatatctttgttgtcttctctgtagtgtaccccgccaaatgtaaggatgcccagcaggt |
| | | acccattttatggatctgagctggggattgatagtgtatctataaatgcactgatcaatttaaaaagcg |
| | | tctaagtttggcacaaacactggggacagtgtttttcctttattcttttatttgatta |
| 655 | Phopivirus strain | gggagtaaacctcaccaccgtttgccgtggtttacggctacctatttttggatgtaaatattaattcctg |
| | NewEngland | caggttcaggtctcttgaattatgtccacgctagtggcactctcttacccataagtgacgccttagcg |
| | | gaacctttctacacttgatgtggttaggggttacattatttccctgggccttctttggccctttttcccctg |
| | | cactatcattctttcttccgggctctcagcatgccaatgttccgaccggtgcgcccgccggggttaa |
| | | ctccatggttagcatggagctgtaggccctaaaagtgctgacactggaactggactattgaagcat |
| | | acactgttaactgaaacatgtaactccaatcgatcttctacaaggggtaggctacgggtgaaacccc |
| | | ttaggttaatactcatattgagagatacttctgataggttaaggttgctggataatggtgagtttaacga |
| | | caaaaaccattcaacagctgtgggccaacctcatcaggtagatgcttttggagccaagtgcgtagg |
| | | ggtgtgtgtggaaatgcttcagtggaaggtgccctcccgaaaggtcgtaggggtaatcaggggca |
| | | gttaggtttccacaattacaatttgaa |
| 656 | Pestivirus strain | gtatacgagattagctcatactcgtgtacaaattggacgtagcaaatttaaaaattcggatagggtcc |
| | Aydin | ccatccagcgacggccgaacggggttaaccatacctctagtaggactagcagacggatggacta |
| | | gccacagtggtgagctccctgggaggtctaagttctgagtacagaacagtcgtcagtagttcaacg |
| | | ctggtaaaccccagccttgagatgctacgtggacgagggcatgcccaagacacaccttaacctgg |
| | | acgggggtcgtccaggtgaaagtacccatctttgggtgctgggagtacagcctgatagggcgctg |
| | | cagaggcccactgacaggctagtataaaaatctctgctgtacatggaac |
| 657 | Quail | tttgcatcagttcgcccctcccctcaccatacctttttcccctctttaggactgatacttggttatgatga |
| | picornavirus | gcagaggatttcgcaagttatgcttcttgataaaaagtaattcacgaatcatgggattatagcctgga |
| | QPV1 | agtgaacactcatgtggcaagtgggttagtagctctccatgttcccatgtgcagtggactgacaaca |
| | | gtgagttcggggttgtgtagtaaagggaaagtattacttacccgcacctgctatacgtggtgtacgta |
| | | ggatacgagttagtagtgcttagcaactttaaactggtgctgaaatattgcaaggtcactgaagttgt |
| | | gaacgcgaacgctccgccactgccatgtatagcgtgcaatgcataaatggtgcactacatgatacg |
| | | agggaatgggaaaccctccatggccgaatgcagggtgacagcctgccggcggatgcctgttgtt |
| | | agtataatccgttgtttgccac |
| 658 | Porcine | acactcatttcccccctccacccttaaggtggttgtatcccctacaccctaccctcccttccacatagg |
| | sapelovirus 1 | acgaataaacggacttgagattaaggcaagtacataaggtatggtttttggatacacttaaatggca |
| | | gtagcgtggcgagctatggaaaaatcgcaattgtcgatagccatgttagcgacgcgcttcggcgtg |
| | | ctcctttggtgattcggcgactggttacaggagagtagacagtgagctatgggcaaacccctacag |
| | | tattacttaggggaatgtgcaattgagacttgacgagcgtctctttgagatgtggcgcatgctcttgg |
| | | cattaccatagtgagcttccaggttgggaaacctggactgggtctatactgcctgatagggtcgcg |
| | | gctggccgcctgtaactagtatagtcagttgaaaacccccc |
| 659 | Porcine | atgacgtataggtgttggctctatgccttggcatttgtattgtcaggagctgtgaccattggcacagc |
| | reproductive and | ccaaaacttgctgcacagaaacacccttctgtgatagcctccttcaggggagcttagggtttgtccct |
| | respiratory | agcaccttgcttccggagttgcactgctttacggtctctccacccctttaacc |
| | syndrome virus 2 | |
| 660 | Porcine | Gaaccttagaagtttacacaaacaaagaccaataggagtccaacacccagttggattgcggtcaa |
| | enterovirus 9 | gcacttctgtttccccggacctagtagtgataggctgtacccacggccgaagatgaacccgtccgtt |
| | | atccggctacctacttcgggaagcctagtaacattctgaagtctctgaggcgtttcgctcagcacga |
| | | ccccggtgtagatcgggctgatgggtctccgcataccccacgggcgaccgtggcggaggccgc |
| | | gttggcggcccgcctatggcgaaagccataggacgcctcttagatgacagggtgtgaagagcct |
| | | actgagctgggtagtagtcctccggcccctgaatgcggctaatcctaaccacggagcgtccacca |
| | | gcaatccagctggcagggcgtcgtaacgggcaactctgtggcggaaccgactactttgggtgtcc |
| | | gtgtttccttttgatcctattttggctgcttatggtgacaacgataagttgttatcataaagctcttgggtt |
| | | ggccacctggaaaaagttatcagtgtttgatattgttcggctctcacgcctaccaataagacaagcc |
| | | ctatatttacttgttgcattttactcgtcagaagaaatcacagagtatcatttggatttgttactcacatta |
| | | aggacaag |
| 661 | Pigeon | tttatttagctgttaagttttatttgtgccgagAccccatagtaggatcttggtgttccacattaagctct |
| | picornavirus B | Cccgaccacacatccaaacgataggcggtgtaagggctccctggctaagtgtttactcattgctag |
| | | ggaagtgttgcgacccgttaccagtaggaatacaggaggtcttagttgcctaaccagataaagtgg |
| | | tgctgaaatattgcaagctcaatgtctggcgaacgacggactaccgttgaactattgttaacgcccg |
| | | cgtgtgtaggcaacacacgggttagtaggtcacttacattgacatccgtgccgggaaagcggatct |
| | | gagctatcgattgcctgatagggtgccggcgggcgcggtacgtgtggtatagtccgctgtcttggg |
| | | gtatggcgtctactcggttttgttgtcgttttggaatgtcccatgtcggagatgtcgttaccggtgtttg |
| | | ctttacttgtgcacgaataagaaaacagagtttggagttttggaagttaatggataacatcaagtttcc |
| | | attgcgttcctcgtgcattttaggccagaaaatcgaccacaaaatcgaga |
| 662 | Picornavirus | aaacgggaggatcggctttggcttatcctcttaatagtctacaaaactgggctgactggtggggga |
| | HK21 | gctactagatccgggagaggactagttaccccgcgtaacttccctttttgtcactccctccacctacc |
| | | ccttccctgtcccttagactcttaagtaggtgtgcaggcctggccccccggaaatgggcaaatcgg |
| | | acgtttgcgtgtagggtcgttctgaaaggtggggcccactccaccgtagtaggatcttcctgtttcac |
| | | gtttaaacctctccgggcaggtatcgctagacactaggctgtataggatggcacgcacttaggtttc |
| | | gcaccttcctagtgcgccaagatcccgcttttgagctcaagtacatggttctttgtaagatgtctaacc |
| | | agaggaggtggtgctgaaatattgcaagccactctggcgaacgtccactgcattgcctggaacag |
| | | gctctctagcgccctccactggtagcgcggtggtgggttagtaggatacctatatggacaggggat |
| | | gcgggaatacccctcactagctagtgactggttgatcgactggcggcggatccagtgatacttgca |
| | | taatccgcagacttgggag |
| 663 | Picornavirales | agccatgaactagtgtgcgattcccacagtgttggtcgaaagccccgcactgtctactcgcatattt |
| | Tottori-HG1 | gactccagtccttccgcagccagcggttaggttctggttaaagtgcattatgtgcacggcgccacg |
| | | cagaactgccagaaatggtaagctgcgcccaacgccaacggtttgtgtatcccgttagtcacacgt |
| | | ttacagctgttcgttaacagtagggttttgtcgacccggaccccttaatgcgcgcgaattcaaccgc |
| | | gcctagtcggcaatggtatcatttaatcccatgcactacgggagaaatttgagaccaaagaattcct |
| | | gagggccactgcttgctctaagtgcaatgcctcgggagacttctgtcaggagcctagcggctttca |
| | | accgcgacagctaactcctgcgggatgtttggtgtccatacttactggcgtcctcacaacgctaagt |
| | | ggatgttgtccacaggtaggcaaacaccgagccccacattcaggagacctgtatgaacgatcctat |
| | | cagcattagagttggaattgggtgtgctaacgtccgcataagtgcaccccgtggtaacgctgggaa |
| | | actatccagcgcaacgtactgtcctcaatgtctagggaaggaccgccctaagcgtacaaccgggc |
| | | catgtgtcgagc |
| 664 | Rodent | ttcaaagaggggtccgggattttcctggtccccctctttgggcacccttggctcgggggtgtgaata |
| | hepatovirus | ccgtgctcgcgtttgccgtgcgttaacggcttcatttatgtttgtttgtctgttttattatgttGgtttgtct |
| | | gtttgttatgttggtattgttcgtgtttaatgttatgaccacattacactccagccaatgaagaacagatg |
| | | gtgcggttattgctggcggaattcctaacgtcctggatccgttggtacgcatcacaaaacaatttgca |
| | | gagagagtggtgaaacggcttgggaatccctgagtacagggaaatcacactgatagctcatcttg |
| | | gctgttttcagtcatggaccttatgcagtgtaatttgggtgtaccccccatagcttaggaggaatgttc |
| | | tgtcttggcactagagtgggacgctgatgcctccgtgtctaggatggtctaagggacagaatgggg |
| | | tgcctctgatgccatactacctgatagggtgctctcacggcctctgcatcttagtgagaagttcaattt |
| | | t |
| 665 | Rinderpest virus | accaaacaaagttgggtaaggatcggtctatcaatgattatgatttagcacacttaggattcaagatc |
| | | ctatcgactggagcaggcttaaggtaaaggttctttaaa |
| 666 | Rabovirus A | ctacggatatttgcatgacccgctttctatcgccccaacaatcccctttgtaaccacaagctttactca |
| | | ggctagcagcccgactagctgtttggaagaaaaggctagggcacacaccaacaacaccgaccc |
| | | cactggtcgaaggccgcttggcaataagactggtggaacagggtcgcctgtagttgtttggaacat |
| | | tctttctaatgactttgtcagcggtgctactcacaccgtaactcttctaccctatccccacgcttgtgga |
| | | actaggaggggatgagtgattcaagtaagtactgtcagaatggtgaaaatgatctgattctgaaacg |
| | | ctatggatccatcgaaagatggggctacacgcctgcggaacaacacatggtaacatgtgccccag |
| | | gggccgaaagccacggtgataggatcacccgtgtagtttgagatcatatcaatgttcatagtctagt |
| | | aagatgatttgaaatctaactgagctgatggctaactgcttgtcttattgcggcctaaggatgtcctgc |
| | | aggtacctttagataaccttaagagactattgatctgagcaggagccaaagtggtctttcccagcttt |
| | | ggttaaaaaacgtctaagccgcggcagggggcgggaggccccctttcctcccaaaacttaatatt |
| | | gattgt |
| 667 | Shingleback | ctgtgagtaccgacaggctcgaagtctattatgaggcgtcgaaacagaaaacctgtaacaactccg |
| | nidovirus 1 | gtttcatctatcactgccgtcaagaggcagaagaggacgaccacgtgtcaccagatcacttgtatct |
| | | gtttcagtcaggaagtcaacttttcgacgaagttcgaccattcatcgacccgctgaaaagcgtagaa |
| | | gtcgatgaagatgagctccaaagagccatcgccgatttcgacaaccaaagtgactgtttccactcct |
| | | tcgagctcgtgaatttcgagctgaaacaacaaatcaacgagaacgagtggtacggttattataatta |
| | | cgacaaccaaaactgcaaagttcagttgccagtcacatgtcgaatcgaggacgtaacctgggatc |
| | | aggtttacgtg |
| 668 | Seneca valley | tttgaaatggggggctgggccctgatgcccagtccttcctttccccttccggggggttaaccggctg |
| | virus | tgtttgctagaggcacagaggggcaacatccaacctgcttttgcggggaacggtgcggctccgatt |
| | | cctgcgtcgccaaaggtgttagcgcacccaaacggcgcacctaccaatgttattggtgtggtctgc |
| | | gagttctagcctactcgtttctcccccgaccattcactcacccacgaaaagtgtgttgtaaccataag |
| | | atttaacccccgcacgggatgtgcgataaccgtaagactggctcaagcgcggaaagcgctgtaac |
| | | cacatgctgttagtccctttatggctgcaagatggctacccacctcggatcactgaactggagctcg |
| | | accctccttagtaagggaaccgagaggccttcgtgcaacaagctccgacacagagtccacgtga |
| | | ctgctaccaccatgagtacatggttctcccctctcgacccaggacttctttttgaatatccacggctcg |
| | | atccagagggtggggcatgacccctagcatagcgagctacagcgggaactgtagctaggcctta |
| | | gcgtgccttggatactgcctgatagggcgacggcctagtcgtgtcggttctataggtagcacatac |
| | | aaat |
| 669 | Sclerotinia | ttgaattaatcttttacgtttacgcgcataaaatcaggacacatctcttgtatactttagtatatcaatgat |
| | sclerotiorum | gtttttgttttatgcgattaatcgtaagagaacttctttccatccgcctgtatgggcgggataataagttc |
| | dsRNA | accgccttggtcgaggcgcaaacttgtatgtgcaaaggtgagctatatgctcgaaatagtcgtaact |
| | mycovirus-L | aacacacagccactacctgtagagctctattgatccggaatcctttagtgggaatgcagagctcaca |
| | | ccggacctgcgggtatcttcggcgttagggacttctgtttcagccttgaatcatttacctttataccttct |
| | | ctgaggcgcctgggccgggcgcgatattaagtacaagtcaaggacatcgcgggtagtggtctaat |
| | | cagccgctagtcctgctggagagttccaacttagttgggtgtggtgcatactagctggatagagtag |
| | | gtatgtattgctaacgtatgccggaggctatccgtcctcggtagaacgtgccgaggagtagtctctg |
| | | cagacccccgaacgcgtggggtctttacttaaatgtaggcggagggagcgctcgtaggtggaac |
| | | gactgcctcccagtcgaatgcaagattttgcacgcggaccagtctgcccggcaattcccgggtg |
| 670 | Yak enterovirus | ctccggcacagccgcaccagtgcactggtacgctagtaccttttcacggggtagtcggtatccccc |
| | | cccgtaacttagaagcatgtaacaaaccgaccaataggtgcgcggcagccagctgcgttgcggtc |
| | | aagcacttctgtctccccggtccgcaaggatcgttacccgcccactccactacgaggagcctagta |
| | | actggccaagtgattgcggagttgcgttcagccacaaccccagtggtagctctggaagatggggc |
| | | tcgcacatcccccgtggtaacacggttgcttgcccgcgtgtgcttccgggttcagtctccgactgttc |
| | | acttcaacatcacgcaaccagccaagagccgattgtgctggagtggtcttcctccggggccgtga |
| | | atgctgctaatcctaacctccgagcgtgtgcgcacaatccagtgttgctacgtcgtaacgcgtaagt |
| | | tggaggcggaacagactactttcggcaccccgtgtttcctttattttattcttattttatggtgacaattg |
| | | cagagatttgtgatattgcgactttaccgttaaacatagcactgcattacctggttgcattccacaaaa |
| | | cttcagagattcctagttcctacattgacctacttgtttatttgaatcttaaatacaaacttgagcaagtg |
| | | aa |
| 671 | Wobbly possum | cggctgtgagtgcttagcatatgctagagtactacagccgggtgttggagtcatatgcactggttgc |
| | disease virus | ctgtataatagtcgggatctgtctgacctacattatctttgggagttgcttatcacgacaattctcgaag |
| | | tgtctgtcgacagcttacccccgattcgacaaggccccttgtccaccgcagacctatcgattttcaac |
| | | gagaacactatcagaggtttaaatttaaaactcaccaaca |
| 672 | Avian | gctttttcaatcccttgtgtcgatgttcccgtatgtcatctggttcatgtaacggtgcaacttctatttttg |
| | orthoreovirus | gtaacgttcactgtcaggcagcgcaaaactcggcgggtggtgatcttcaagcgacttcctctcttgtt |
| | segment S1 | gcttattggccctacttggctgcgggcggtggtctgctcgttgttattattataattgttggcgctgtttg |
| | | ttgttgcaaggctaaggttaaagcggacgcagcccgaaacgttttctaccgagagctgttcgcactt |
| | | aattcgggtaaaagtgatgcaggacctccgatttaccaggtttagtgtacgacgatttgagttttcac |
| | | ctttcgtcttagaggagtgcactactccatctttcacgactataactaataccgatccggctctctactt |
| | | taacattgagtttccgtcaagtcatcgtctctcccccttcattccagaactgttgtctcagccttgtacc |
| | | gttcacgtttcattgattcggagattcgctctctgtgcaaccttatctagtatttgtgaatacgactgtgc |
| | | gctactgccatccatcaacgctattacgacgatccctacaccaggtgcgtcatcatctctgattgttc |
| | | attggg |
| 673 | Caprine | ggggcctcggccccctcaccctcttttccggtggccacgcccgggccaccgatacttcccttcact |
| | Kobuvirus d10 | ccttcgggactgttggggaggaacacaacagggctcccctgttttcccattccttcccccttttccca |
| | | accccaaccgccgtatctggtggcggcaagacacacgggtctttccctctaaagcacaattgtgtg |
| | | tgtgtcccaggtcctcctgcgtacggtgcgggagtgctcccacccaactgttgtaagcctgtccaa |
| | | cgcgtcgtcctggcaagactatgacgtcgcatgttccgctgcggatgccgaccgggtaaccggtt |
| | | ccccagtgtgtgtagtgcgatcttccaggtcctcctggttggcgttgtccagaaactgcttcaggtaa |
| | | gtggggtgtgcccaatccctacaaaggttgattctttcaccaccttaggaatgctccggaggtaccc |
| | | cagcaacagctgggatctgaccggaggctaattgtctacgggtggtgtttcctttttcttttcacacaa |
| | | ctctactgctgacaactcactgactatccacttgctctcttgtgcctttctgctctggttcaagttccttg |
| | | attgtttttgactgcttttcactgcttttcttctcacaatccttgctcagttcaaagtc |
| 674 | Caprine | ccccctcaccctcttttccggtggccacgcccgggccaccgatacttcccttcactccttcgggact |
| | Kobuvirus d20 | gttggggaggaacacaacagggctcccctgttttcccattccttcccccttttcccaaccccaaccg |
| | | ccgtatctggtggcggcaagacacacgggtctttccctctaaagcacaattgtgtgtgtgtcccagg |
| | | tcctcctgcgtacggtgcgggagtgctcccacccaactgttgtaagcctgtccaacgcgtcgtcct |
| | | ggcaagactatgacgtcgcatgttccgctgcggatgccgaccgggtaaccggttccccagtgtgt |
| | | gtagtgcgatcttccaggtcctcctggttggcgttgtccagaaactgcttcaggtaagtggggtgtg |
| | | cccaatccctacaaaggttgattctttcaccaccttaggaatgctccggaggtaccccagcaacag |
| | | ctgggatctgaccggaggctaattgtctacgggtggtgtttcctttttcttttcacacaactctactgct |
| | | gacaactcactgactatccacttgctctcttgtgcctttctgctctggttcaagttccttgattgtttttga |
| | | ctgcttttcactgcttttcttctcacaatccttgctcagttcaaagtc |
| 675 | Caprine | ctcttttccggtggccacgcccgggccaccgatacttcccttcactccttcgggactgttggggagg |
| | Kobuvirus d30 | aacacaacagggctcccctgttttcccattccttcccccttttcccaaccccaaccgccgtatctggt |
| | | ggcggcaagacacacgggtctttccctctaaagcacaattgtgtgtgtgtcccaggtcctcctgcgt |
| | | acggtgcgggagtgctcccacccaactgttgtaagcctgtccaacgcgtcgtcctggcaagactat |
| | | gacgtcgcatgttccgctgcggatgccgaccgggtaaccggttccccagtgtgtgtagtgcgatct |
| | | tccaggtcctcctggttggcgttgtccagaaactgcttcaggtaagtggggtgtgcccaatccctac |
| | | aaaggttgattctttcaccaccttaggaatgctccggaggtaccccagcaacagctgggatctgac |
| | | cggaggctaattgtctacgggtggtgtttcctttttcttttcacacaactctactgctgacaactcactg |
| | | actatccacttgctctcttgtgcctttctgctctggttcaagttccttgattgtttttgactgcttttcactgc |
| | | ttttcttctcacaatccttgctcagttcaaagtc |
| 676 | Caprine | gtggccacgcccgggccaccgatacttcccttcactccttcgggactgttggggaggaacacaac |
| | Kobuvirus d40 | agggctcccctgttttcccattccttcccccttttcccaaccccaaccgccgtatctggtggcggcaa |
| | | gacacacgggtctttccctctaaagcacaattgtgtgtgtgtcccaggtcctcctgcgtacggtgcg |
| | | ggagtgctcccacccaactgttgtaagcctgtccaacgcgtcgtcctggcaagactatgacgtcgc |
| | | atgttccgctgcggatgccgaccgggtaaccggttccccagtgtgtgtagtgcgatcttccaggtc |
| | | ctcctggttggcgttgtccagaaactgcttcaggtaagtggggtgtgcccaatccctacaaaggttg |
| | | attctttcaccaccttaggaatgctccggaggtaccccagcaacagctgggatctgaccggaggct |
| | | aattgtctacgggtggtgtttcctttttcttttcacacaactctactgctgacaactcactgactatccact |
| | | tgctctcttgtgcctttctgctctggttcaagttccttgattgtttttgactgcttttcactgcttttcttctca |
| | | caatccttgctcagttcaaagtc |
| 677 | Caprine | ccgggccaccgatacttcccttcactccttcgggactgttggggaggaacacaacagggctcccc |
| | Kobuvirus d50 | tgttttcccattccttcccccttttcccaaccccaaccgccgtatctggtggcggcaagacacacgg |
| | | gtctttccctctaaagcacaattgtgtgtgtgtcccaggtcctcctgcgtacggtgcgggagtgctcc |
| | | cacccaactgttgtaagcctgtccaacgcgtcgtcctggcaagactatgacgtcgcatgttccgctg |
| | | cggatgccgaccgggtaaccggttccccagtgtgtgtagtgcgatcttccaggtcctcctggttgg |
| | | cgttgtccagaaactgcttcaggtaagtggggtgtgcccaatccctacaaaggttgattctttcacca |
| | | ccttaggaatgctccggaggtaccccagcaacagctgggatctgaccggaggctaattgtctacg |
| | | ggtggtgtttcctttttcttttcacacaactctactgctgacaactcactgactatccacttgctctcttgt |
| | | gcctttctgctctggttcaagttccttgattgtttttgactgcttttcactgcttttcttctcacaatccttgct |
| | | cagttcaaagtc |
| 678 | Picornavirales sp. | tttgctcagcgtaacttctccgggttacgtggagaccaaaaggctacggagactcgggctacggc |
| | isolate RtMruf- | cctggagcacctaggtgctcctaaagacgttagaagttgtacaaactcgcccaatagggcccccc |
| | PicoV | aaccaggggggtagcgggcaagcacttctgtttccccggtatgatctcataggctgtacccacgg |
| | | ctgaaagagagattatcgttacccgcctcactacttcgagaagcccagtaatggttcatgaagttgat |
| | | ctcgttgacccggtgtttcccccacaccagaaacctgtgatgggggtggtcatcccggtcatggcg |
| | | acatgacggacctccccgcgccggcacagggcctcttcggaggacgagtgacatggattcaacc |
| | | gtgaagagcctattgagctagtgttgattcctccgcccccgtgaatgcggctaatcccaactccgga |
| | | gcaggcgggcccaaaccagggtctggcctgtcgtaacgcgaaagtctggagcggaaccgacta |
| | | ctttcgggaaggcgtgtttccttttgttccttttatcaagttttatggtgacaactcctggtagacgttttat |
| | | tgcgtttattgagagatttccaacaattgaacagactagaaccacttgttttatcaaaccctcacagaa |
| | | taagataaca |
| 679 | Apodemus | ttactcagcgtaactactccgggttacgtgatgaagaagaggctacggagattctcgggctacggc |
| | agrarius | cctggagccactccggctcctaaagatttagaagtttgagcacacccgcccactagggcccccca |
| | picornavirus | tccaggggggcaacgggcaagcacttctgtttccccggtatgatctgataggctgtaaccacggct |
| | strain Longquan- | gaaacagagattatcgttatccgcttcactacttcgagaagcctagtaatgatgggtgaaattgaatc |
| | Aa118 | cgttgatccggtgtctcccccacaccagaaactcatgatgagggttgccatcccggctacggcga |
| | | cgtagcgggcatccctgcgctggcatgaggcctcttaggaggacggatgatatggatcttgtcgtg |
| | | aagagcctattgagctagtgtcgactcctccgcccccgtgaatgcggctaatcctaaccccggagc |
| | | aggtgggtccaatccagggcctggcctgtcgtaatgcgtaagtctgggacggaaccgactactttc |
| | | gggaaggcgtgtttccatttgttcattatttgtgtgtttatggtgacaactctgggtaaacgttctattgc |
| | | gtttattgagagattcccaacaattgaacaaacgagaactacctgttttattaaatttacacagagaag |
| | | aattaca |
| 680 | Niviventer | ccctttcataaccccccccttttaacccaacccttcgtaaccgtacgcttcactcgcctttgggtatag |
| | confucianus | cggcccaatgtgctgaagaaaaggatacgctataaggggccaacgggtggtggcccttaagacc |
| | picomavirus | acccaacctagaagcttgtacactcgggcaatagtgaggcccacatccagtgggtcaagcccaaa |
| | | gcattcttgttccccggtatgatctcataagctgtacccacggctgaaagagtgattatcgttatccca |
| | | ctcagtacttcggagagcctagtacaccacttggaaatggaagtctgtgatccggggttgaccctg |
| | | aaccccagaaactcatgatgaggctaaccttcccgaacacggcgacgtgtggttagcctgcgctg |
| | | gcatgaggcctctttgtaggcagactgaaatggaagggtgacgaagagccgactgagctactgttt |
| | | tattcctccggccccctgaatgcggctaatcctaactcctggtccagtacttgtaacccaacaggtg |
| | | gctggtcgtaatgcgtaagccgggagcggaaccgactactttggggcgtccgtgtttctcaatatta |
| | | ttcatttctagcttatggtgacaatttatgattgcagagattgtgctgtatttgtgtctgagagaagaagt |
| | | aacaat |
| 681 | Bat picomavirus | tttcaaaaggccctgggcatacggcgttattcgtaacgtcgtatgtccagggcggtagcatcaggc |
| | isolate BtRs- | caaggcctgatgctaccacgtgtggactaaaccacacactcttcttgtgacacgttgtgtcacctatc |
| | PicoV | cctttcttggtaacttagaagcttgtacacttacgcacgtaggtgccccacatccagtggggtttgtg |
| | | caaagcaatcttgttccccggtaaaccctgataggctgtaaccacggccgaaacaaggtttgtcgtt |
| | | acccgactcactactacacaaagcctagtaaagttcaatgaaagtgcgcagcgtgatccggtcaaa |
| | | acccccttgaccagaaacacatgatgagggtcaccaacccccactggcgacagtgtggtgtccct |
| | | gcgttggcatgtggcctcgtagaggcgttgcaatctggatttgctccgaagagccccgtgtgctagt |
| | | gtttatacctccggccccttgaatgcggctaatcctaacccccgagcatgtacacacaagccagtgt |
| | | gtagcatgtcgtaatgagcaatttggggatggaaccgactactttagggtgtccgtgtttctcattatt |
| | | ctttgtttgatgtttt |
| 682 | Rhinolophus | ttttttttctcaggcggtagcatccagccaaggcctgatgctaccaacgtgtgactaaaccacactct |
| | picornavirus | ctttttgtgatacattgtgtcacctatccctttcttggtaacttagaagcttgtacacccacgcacgtag |
| | strain Guizhou- | gtaccccacatccagtggggtttgtgcaaagcattcttgttccccggtaaaccctgataggctgtaa |
| | Rr100 | ccacggctgaaacaaggtttgtcgttacccgactcactactacgcaaagcctagtaaagttcaatga |
| | | aagtgcgcagcgtgatccggtcaaaacccccttgaccagaaacacatgatgagggtcaccaacc |
| | | cccactggcgacagtgtggtgtccctgcgttggcatgtggcctcatagaggcgttgcaatctggatt |
| | | tgctccgaagagccccgtgtgctagtgtttatacctccggccccttgaatgcggctaatcctaaccc |
| | | ccgagcatgtacacacacgccagtgtgcagcatgtcgtaatgagcaatttggggatggaaccgac |
| | | tactttagggtgtccgtgtttctcattattctttgtttgatgttttatggtgacaaca |
| 683 | Rhinolophus | cggaacgttgtatgctcagggcgtaggcaccacccacgggtggtgcctacacgtgtggactaaac |
| | picornavirus | cacacactcttttcagcacttagtgctgctatctctttttgtaacttagaagtttgtacacaatgcgttag |
| | strain Henan- | ggccacacatccagtgtggtatcgcaaagcacttctgtttccccggtgctagtaggagggtggctg |
| | Rf265 | ctccacggccacttgccgaacccatcgttacccgactcattacttcgcaaagcctagtaacccagtt |
| | | gaagcaagcccggcgtgttccggtcaggaaaaaccccccctggccagaaacatgtgatgagggt |
| | | gggctatccccactggtgacagtgagccctccctgcgttggcacatggcccgatctgggcgtggtt |
| | | cttgtggatgctgccgaagagccccgtgagctagtgtttataccgccggcctcgtgaatgcggcta |
| | | accctaaccccggagcagaggctactgaagccacagtagtcgctgtcgtaacgagtaattctggg |
| | | atgggaccgactactttcgagtgtccgtgtttcctttattcttttattgttgtttatggtgacaaac |
| 684 | Human | cccctaggatccactggatgtcagtacactggtatcgtggtacctttgtacgcctgttttataccccctt |
| | enterovirus C105 | ccccgcaactttagaagcatcaaaagcaccgctcaatagtcaccacacccccagtgtggtttcgag |
| | | caagcacttctgttttcccggttgcgtcccatatgctgtgcaaacggcaaaaagggacaatatcgtta |
| | | cccgcttgtatactacgggaaacctagtaccaccattgattgtgttgagagttgcgctcatcacctttc |
| | | cccggtgtagctcaggccgatgaggctcagaatcccccacaggtgactgtgtctgagcctgcgtt |
| | | ggcggcctgccctcgccttatggcgtgggacgcttgatacatgacatggtgcgaagagtctactgt |
| | | gctatgcaagagtcctccggcccctgaatgtggctaatcctaaccactgatcccacgcacgcaaac |
| | | cagtgtgtagtgggtcgtaacgcgcaagtcggtggcggaaccaactactttgggtgaccgtgtttc |
| | | ctttattacttattgaatgtttatggtgacaattgtttgattcagttgttgccattctctacattcatttaccca |
| | | gcatcaaaccaattgaactgttaca |
| 685 | Human | agtctggacatccctcaccggcgacggtggtccaggctgcgctggcggcctacctgtggtccaaa |
| | poliovirus 1 | gccacgggacgctacatgtgaacaaggtgtgaagagcctattgagctacaaaagagtcctccgg |
| | strain | cccctgaatgcggctaatcccaaccacggatcaagggtgcacaaaccagtgtacaccttgtcgta |
| | NIE1116623 | acgcgcaagtctgtggcggaaccgactactttgggtgtccgtgtttcctttttaattttgatggctgctt |
| | | atggtgacaatcatagattgttatcataaagctaattggattggccatccggtgagagtgaaatatatt |
| | | gtttacctccctgttgggtttactctaactaacttctccatttataaacttgtcatcacagttttaataatta |
| | | gaagtgcagtttaca |
| 686 | Human | tttaaaacagcctgggggttgttcccacccccagggcccactgggcgttagtactctggtatcgcg |
| | enterovirus 109 | gtaccttagtatgcctgttttatgtctcctttcccccgcaactttagaagtaatcaagttatggctcaaca |
| | | gtcgccacacccccagtgtggttccgagcaagcacttctgttccccggttgcgtcttatatgctgtgt |
| | | gaacggcagaaagggacaatatcgttatccgctcaactactacgggaagcctagtaccaccatgg |
| | | attgacctgaaagttgcgttcagcgcacccccagcgcagctcaggccgatgaggctccgaatacc |
| | | ccacgggcgaccgtgtcggagcctgcgttggcggcctgcccacgttgcaaaacgtgggacgctc |
| | | atttcatgacatggtgcgaagagcctactgtgctagttgagagtcctccggcccctgaatgtggata |
| | | atcctaaccactgaacctacgggcgcaaaccagcgtctggtaggccgtaacgcgcaagtcggtg |
| | | gcggaaccaactactttgggtgtccgtgtttccttttatctttttgaatgtttatggtgacaattgttgtgta |
| | | cagttgttaccatagtttgcattcagaaataaacctaacactttccaattatttgttaca |
| 687 | Human | ttgtgcgcctgttttatattccccccccgcaacttagaagcacgaaaccaagttcaatagaaggggg |
| | poliovirus 2 | tacaaaccagtaccaccacgaacaagcacttctgtttccccggtgacattgcatagactgctcacgc |
| | strain | ggttgaaagtgatcgatccgttacccgcttgtgtacttcgaaaagcctagtattgccttggaatcttcg |
| | NIE0811460 | acgcgttgcgctcagcacccgaccccggggtgtagcttaggctgatgagtctggacattcctcacc |
| | | ggtgacggtggtccaggctgcgttggcggcctacctatggctaatgccataggacgctagatgtg |
| | | aacaaggtgtgaagagcctattgagctacataagagtcctccggcccctgaatgcggctaatccta |
| | | accacggagcaggcggtcgcaaaccagtgactagcttgtcgtaacgcgcaagtctgtggcggaa |
| | | ccgactactttgggtgtccgtgtttcctgttatttttattatggctgcttatggtgacaatcagagattgtt |
| | | atcataaagcgaattggattggccatccggtgagtgttgtgtcaggtatacaactgtttgttggaacc |
| | | actgtgttagtttaacctctctttcaaccaattagtcaaaaacaatacgaagatagaacaacaatacta |
| | | ca |
| 688 | Bovine | ttttctcccctccccctccaactaccttttccccctcttgtaacgctagaagtttgtgcaaaccgcctgt |
| | picornavirus | agggtactgcaatccagcagtgcataggctaagcttttcttgttaccccaccccacattatactgagg |
| | | aggattgtgaaattgtgttagtatgggttagtagcggtgacccgggtaaccccaacccagaaactc |
| | | acggatgagatgaacaggaccccacatggtaacgtgtgtgttcgtctgccccgcaaggtgaggcc |
| | | gtgagagctttgcacgcgaaaaccttgaaaacccaaaagtaccttgagctcttcgctattttgtgtttc |
| | | ctccaggaccctgaatgcggctaaacctaacccgcgatccgcacgtagcaacccagctagagtgt |
| | | ggtcgtaatgcgcaagttgcgggcggtaccgactactttggtgttcctgtgtttcctttattttattttga |
| | | atttttatggtgacaacagctagaaaataagagtgaac |
| 689 | Human | acccttgtacgcctgttttatactcccctccccgtaacttagaagaaacaaaataagttcaataggag |
| | poliovirus 1 | ggggtacaaaccagtaccaccacgaacaagcacttctgtctccccggtgacattgcatagactgtc |
| | strain | cccacggttgaaagcaattgatccgttacccgctcttgtacttcgagaagcctagtaccatcttggaa |
| | EQG1419328 | tcatcgatgcgttgcgctccacactcagtcccagagtgtagcttaggctgatgagtctggacattcct |
| | | caccggcgacggtggtccaggctgcgttggcggcctacctgtggcccaaagccacaggacgct |
| | | agatgtgaacaaggtgtgaagagcctattgagctataagagagtcctccggcccctgaatgcggc |
| | | taatcccaaccacggatcaagggtgcacgaaccagtgtataccttgtcgtaacgcgcaagtccgtg |
| | | gcggaaccgactactttgggtgaccgtgtttccttttattatttcaatggctgcttatggtgacaatcatt |
| | | gattgttatcataaagcgaattggactggccatccggtgaaagtgaaacatattgtttgcctcctcgtt |
| | | gggtctacttcaaccaatctttacttacaatcttaccactacagttttgctggttagaagtgtgtttcacg |
| 690 | Human | ttgtgcgcctgttttatactcccctcccgcaacttagaagcacgaaaccaagttcaatagaaggggg |
| | poliovirus 2 | tacaaaccagtaccactacgaacaagcacttctgtttccccggtgacattgcatagactgctcacgc |
| | isolate IS_061 | ggttgaaagtgatcgatccgttacccgcttgtgtacttcgaaaagcctagtatcgccttggaatcttc |
| | | gacgcgttgcgctcagcacccgaccccggggtgtagcttaggccgatgagtctggacattcctca |
| | | ccggtgacggtggtccaggctgcgttggcggcctacctatggctaacgccataggacgttagatg |
| | | tgaacaaggtgtgaagagcctattgagctacataagagtcctccggcccctgaatgcggctaatcc |
| | | taaccacggagcaggcggtcgcgaaccagtgactggcttgtcgtaacgcgcaagtctgtggcgg |
| | | aaccgactactttgggtgtccgtgtttcctgttatttttatcatggctgcttatggtgacaatcagagatt |
| | | gttatcataaagcgaattggattggccatccggtgagtgttgtgtcaggtatacaactgtttgttggaa |
| | | ccactgtgttagctttgcttctcatttaaccaattaatcaaaaacaatacgaggataaaacaacaatac |
| | | taca |
| 691 | Coxsackievirus | cctttgtgcgcctgttttatgcccccttcccccaattgaaacttagaagttacacacaccgatcaacag |
| | B5 | cgggcgtggcataccagccgcgtcttgatcaagcactcctgtttccccggaccgagtatcaataga |
| | | ctgctcacgcggttgaaggagaaaacgttcgttacccggctaactacttcgagaaacctagtagca |
| | | tcatgaaagttgcgaagcgtttcgctcagcacatccccagtgtagatcaggtcgatgagtcaccgc |
| | | attccccacgggcgaccgtggcggtggctgcgttggcggcctgcctacggggcaacccgtagg |
| | | acgcttcaatacagacatggtgcgaagagtcgattgagctagttagtagtcctccggcccctgaatc |
| | | cggctaatcctaactgcggagcacataccctcaacccagggggcattgtgtcgtaacgggtaact |
| | | ctgcagcggaaccgactactttgggtgtccgtgtttccttttattcttataatggctgcttatggtgaca |
| | | attgaaagattgttaccatatagctattggattggccatccggtgtctaacagagctattatatacctct |
| | | ttgttggatttgtaccacttgatctaaaggaagtcaagacactacaattcatcatacaattgaacacag |
| | | caaa |
| 692 | Coxsackievirus | tttgtgcgcctgttttacaacccttccccaacttgtaacgtagaagtaatacacactactgatcaatag |
| | A10 | caggcatggcgcgccagtcatgtctcgatcaagcacttctgttcccccggactgagtatcaataga |
| | | ctgctcacgcggttgaaggagaaaacgttcgttacccggctaactacttcgagaaacctagtagca |
| | | ccatagaagctgcagagtgtttcgctcagcacttcccccgtgtagatcaggctgatgagtcactgca |
| | | atccccacgggtgaccgtggcagtggctgcgttggcggcctgcctatggggcaacccataggac |
| | | gctctaatgtggacatggtgcgaagagtctattgagctagttagtagtcctccggcccctgaatgcg |
| | | gctaatcctaactgcggagcacatgccttcaacccagaaggtagtgtgtcgtaacgggcaactctg |
| | | cagcggaaccgactactttgggtgtccgtgtttctttttattcctatattggctgcttatggtgacaatca |
| | | cggaattgttgccatatagctattggattggccatccggtgtctaatagagctattgtgtacctatttgtt |
| | | ggatttactccgctatcacataaatctctgaacactttgtgctttatattgaacttaaacacccgaaa |

In some embodiments, an IRES of the invention is an IRES having a sequence as listed in Table 17 (SEQ ID NOs: 1-72 and 348-389). In some embodiments, an IRES is a Salivirus IRES. In some embodiments, an IRES is a Salivirus SZ1 IRES. In some embodiments, an IRES is a AP1.0 (SEQ ID NO:348). In some embodiments, an IRES is a CK1.0 (SEQ ID NO:349). In some embodiments, an IRES is a PV1.0 (SEQ ID NO:350). In some embodiments, an IRES is a SV1.0 (SEQ ID NO:351).

TABLE 18
Anabaena permutation site 5′ intron fragment sequences.

| SEQ | Permutation | |
|---|---|---|
| ID NO | site | Sequence |
| 73 | L2-1 | GAAGAAATTCTTTAAGTGGATGCTCTCAAACTCAGGGAAACC |
| | | TAAATCTAGTTATAGACAAGGCAATCCTGAGCCAAGCCGAAG |
| | | TAGTAATTAGTAAGTTAACAATAGATGACTTACAACTAATCG |
| | | GAAGGTGCAGAGACTCGACGGGAGCTACCCTAACGTCAAGAC |
| | | GAGGGTAAAGAGAGAGTCCAATTCTCAAAGCCAATAGGCAGT |
| | | AGCGAAAGCTGCAAGAGAATGAAAATCCGT |
| 74 | L2-2 | AAGAAATTCTTTAAGTGGATGCTCTCAAACTCAGGGAAACCT |
| | | AAATCTAGTTATAGACAAGGCAATCCTGAGCCAAGCCGAAGT |
| | | AGTAATTAGTAAGTTAACAATAGATGACTTACAACTAATCGG |
| | | AAGGTGCAGAGACTCGACGGGAGCTACCCTAACGTCAAGACG |
| | | AGGGTAAAGAGAGAGTCCAATTCTCAAAGCCAATAGGCAGTA |
| | | GCGAAAGCTGCAAGAGAATGAAAATCCGT |
| 75 | L2-3 | AGAAATTCTTTAAGTGGATGCTCTCAAACTCAGGGAAACCTA |
| | | AATCTAGTTATAGACAAGGCAATCCTGAGCCAAGCCGAAGTA |
| | | GTAATTAGTAAGTTAACAATAGATGACTTACAACTAATCGGA |
| | | AGGTGCAGAGACTCGACGGGAGCTACCCTAACGTCAAGACGA |
| | | GGGTAAAGAGAGAGTCCAATTCTCAAAGCCAATAGGCAGTAG |
| | | CGAAAGCTGCAAGAGAATGAAAATCCGT |
| 76 | L5-1 | GTTATAGACAAGGCAATCCTGAGCCAAGCCGAAGTAGTAATT |
| | | AGTAAGTTAACAATAGATGACTTACAACTAATCGGAAGGTGC |
| | | AGAGACTCGACGGGAGCTACCCTAACGTCAAGACGAGGGTAA |
| | | AGAGAGAGTCCAATTCTCAAAGCCAATAGGCAGTAGCGAAAG |
| | | CTGCAAGAGAATGAAAATCCGT |
| 77 | L5-2 | TTATAGACAAGGCAATCCTGAGCCAAGCCGAAGTAGTAATTA |
| | | GTAAGTTAACAATAGATGACTTACAACTAATCGGAAGGTGCA |
| | | GAGACTCGACGGGAGCTACCCTAACGTCAAGACGAGGGTAAA |
| | | GAGAGAGTCCAATTCTCAAAGCCAATAGGCAGTAGCGAAAGC |
| | | TGCAAGAGAATGAAAATCCGT |
| 78 | L5-3 | TATAGACAAGGCAATCCTGAGCCAAGCCGAAGTAGTAATTAG |
| | | TAAGTTAACAATAGATGACTTACAACTAATCGGAAGGTGCAG |
| | | AGACTCGACGGGAGCTACCCTAACGTCAAGACGAGGGTAAAG |
| | | AGAGAGTCCAATTCTCAAAGCCAATAGGCAGTAGCGAAAGCT |
| | | GCAAGAGAATGAAAATCCGT |
| 79 | L5-4 | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCAAGACGAGGGTAAAGA |
| | | GAGAGTCCAATTCTCAAAGCCAATAGGCAGTAGCGAAAGCTG |
| | | CAAGAGAATGAAAATCCGT |
| 80 | L5-5 | TAGACAAGGCAATCCTGAGCCAAGCCGAAGTAGTAATTAGTA |
| | | AGTTAACAATAGATGACTTACAACTAATCGGAAGGTGCAGAG |
| | | ACTCGACGGGAGCTACCCTAACGTCAAGACGAGGGTAAAGAG |
| | | AGAGTCCAATTCTCAAAGCCAATAGGCAGTAGCGAAAGCTGC |
| | | AAGAGAATGAAAATCCGT |
| 81 | L6-1 | ACAATAGATGACTTACAACTAATCGGAAGGTGCAGAGACTCG |
| | | ACGGGAGCTACCCTAACGTCAAGACGAGGGTAAAGAGAGAG |
| | | TCCAATTCTCAAAGCCAATAGGCAGTAGCGAAAGCTGCAAGA |
| | | GAATGAAAATCCGT |
| 82 | L6-2 | CAATAGATGACTTACAACTAATCGGAAGGTGCAGAGACTCGA |
| | | CGGGAGCTACCCTAACGTCAAGACGAGGGTAAAGAGAGAGTC |
| | | CAATTCTCAAAGCCAATAGGCAGTAGCGAAAGCTGCAAGAGA |
| | | ATGAAAATCCGT |
| 83 | L6-3 | AATAGATGACTTACAACTAATCGGAAGGTGCAGAGACTCGAC |
| | | GGGAGCTACCCTAACGTCAAGACGAGGGTAAAGAGAGAGTCC |
| | | AATTCTCAAAGCCAATAGGCAGTAGCGAAAGCTGCAAGAGAA |
| | | TGAAAATCCGT |
| 84 | L6-4 | ATAGATGACTTACAACTAATCGGAAGGTGCAGAGACTCGACG |
| | | GGAGCTACCCTAACGTCAAGACGAGGGTAAAGAGAGAGTCCA |
| | | ATTCTCAAAGCCAATAGGCAGTAGCGAAAGCTGCAAGAGAAT |
| | | GAAAATCCGT |
| 85 | L6-5 | TAGATGACTTACAACTAATCGGAAGGTGCAGAGACTCGACGG |
| | | GAGCTACCCTAACGTCAAGACGAGGGTAAAGAGAGAGTCCAA |
| | | TTCTCAAAGCCAATAGGCAGTAGCGAAAGCTGCAAGAGAATG |
| | | AAAATCCGT |
| 86 | L6-6 | AGATGACTTACAACTAATCGGAAGGTGCAGAGACTCGACGGG |
| | | AGCTACCCTAACGTCAAGACGAGGGTAAAGAGAGAGTCCAAT |
| | | TCTCAAAGCCAATAGGCAGTAGCGAAAGCTGCAAGAGAATGA |
| | | AAATCCGT |
| 87 | L6-7 | GATGACTTACAACTAATCGGAAGGTGCAGAGACTCGACGGGA |
| | | GCTACCCTAACGTCAAGACGAGGGTAAAGAGAGAGTCCAATT |
| | | CTCAAAGCCAATAGGCAGTAGCGAAAGCTGCAAGAGAATGA |
| | | AAATCCGT |
| 88 | L6-8 | ATGACTTACAACTAATCGGAAGGTGCAGAGACTCGACGGGAG |
| | | CTACCCTAACGTCAAGACGAGGGTAAAGAGAGAGTCCAATTC |
| | | TCAAAGCCAATAGGCAGTAGCGAAAGCTGCAAGAGAATGAA |
| | | AATCCGT |
| 89 | L6-9 | TGACTTACAACTAATCGGAAGGTGCAGAGACTCGACGGGAGC |
| | | TACCCTAACGTCAAGACGAGGGTAAAGAGAGAGTCCAATTCT |
| | | CAAAGCCAATAGGCAGTAGCGAAAGCTGCAAGAGAATGAAA |
| | | ATCCGT |
| 90 | L8-1 | CAAGACGAGGGTAAAGAGAGAGTCCAATTCTCAAAGCCAATA |
| | | GGCAGTAGCGAAAGCTGCAAGAGAATGAAAATCCGT |
| 91 | L8-2 | AAGACGAGGGTAAAGAGAGAGTCCAATTCTCAAAGCCAATAG |
| | | GCAGTAGCGAAAGCTGCAAGAGAATGAAAATCCGT |
| 92 | L8-3 | AGACGAGGGTAAAGAGAGAGTCCAATTCTCAAAGCCAATAGG |
| | | CAGTAGCGAAAGCTGCAAGAGAATGAAAATCCGT |
| 93 | L8-4 | GACGAGGGTAAAGAGAGAGTCCAATTCTCAAAGCCAATAGGC |
| | | AGTAGCGAAAGCTGCAAGAGAATGAAAATCCGT |
| 94 | L8-5 | ACGAGGGTAAAGAGAGAGTCCAATTCTCAAAGCCAATAGGCA |
| | | GTAGCGAAAGCTGCAAGAGAATGAAAATCCGT |
| 95 | L9a-1 | AATAGGCAGTAGCGAAAGCTGCAAGAGAATGAAAATCCGT |
| 96 | L9a-2 | ATAGGCAGTAGCGAAAGCTGCAAGAGAATGAAAATCCGT |
| 97 | L9a-3 | TAGGCAGTAGCGAAAGCTGCAAGAGAATGAAAATCCGT |
| 98 | L9a-4 | AGGCAGTAGCGAAAGCTGCAAGAGAATGAAAATCCGT |
| 99 | L9a-5 | GGCAGTAGCGAAAGCTGCAAGAGAATGAAAATCCGT |
| 100 | L9-1 | GAAAGCTGCAAGAGAATGAAAATCCGT |
| 101 | L9-2 | AAAGCTGCAAGAGAATGAAAATCCGT |
| 102 | L9-3 | AAGCTGCAAGAGAATGAAAATCCGT |
| 103 | L9-4 | AGCTGCAAGAGAATGAAAATCCGT |
| 104 | L9-5 | GCTGCAAGAGAATGAAAATCCGT |
| 105 | L9-6 | CTGCAAGAGAATGAAAATCCGT |
| 106 | L9-7 | AAGAGAATGAAAATCCGT |
| 107 | L9-8 | AGAGAATGAAAATCCGT |
| 108 | L9-9 | GAGAATGAAAATCCGT |
| 109 | L9a-6 | GCAGTAGCGAAAGCTGCAAGAGAATGAAAATCCGT |
| 110 | L9a-7 | AGTAGCGAAAGCTGCAAGAGAATGAAAATCCGT |
| 111 | L9a-8 | GTAGCGAAAGCTGCAAGAGAATGAAAATCCGT |

In some embodiments, a 5′ intron fragment is a fragment having a sequence listed in Table 18. Typically, a construct containing a 5′ intron fragment listed in Table 18 will contain a corresponding 3′ intron fragment as listed in Table 19 (e.g., both representing fragments with the L9a-8 permutation site).

TABLE 19
Anabaena permutation site 3’ intron fragment sequences.

| SEQ | Permutation | |
|---|---|---|
| ID NO | site | Sequence |
| 112 | L2-1 | ACGGACTTAAATAATTGAGCCTTAAA |
| 113 | L2-2 | ACGGACTTAAATAATTGAGCCTTAAAG |
| 114 | L2-3 | ACGGACTTAAATAATTGAGCCTTAAAGA |
| 115 | L5-1 | ACGGACTTAAATAATTGAGCCTTAAAGAAGAAATTCTTTAAG |
| | | TGGATGCTCTCAAACTCAGGGAAACCTAAATCTA |
| 116 | L5-2 | ACGGACTTAAATAATTGAGCCTTAAAGAAGAAATTCTTTAAG |
| | | TGGATGCTCTCAAACTCAGGGAAACCTAAATCTAG |
| 117 | L5-3 | ACGGACTTAAATAATTGAGCCTTAAAGAAGAAATTCTTTAAG |
| | | TGGATGCTCTCAAACTCAGGGAAACCTAAATCTAGT |
| 118 | L5-4 | ACGGACTTAAATAATTGAGCCTTAAAGAAGAAATTCTTTAAG |
| | | TGGATGCTCTCAAACTCAGGGAAACCTAAATCTAGTT |
| 119 | L5-5 | ACGGACTTAAATAATTGAGCCTTAAAGAAGAAATTCTTTAAG |
| | | TGGATGCTCTCAAACTCAGGGAAACCTAAATCTAGTTA |
| 120 | L6-1 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | A |
| 121 | L6-2 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AA |
| 122 | L6-3 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AAC |
| 123 | L6-4 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACA |
| 124 | L6-5 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAA |
| 125 | L6-6 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAAT |
| 126 | L6-7 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATA |
| 127 | L6-8 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAG |
| 128 | L6-9 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGA |
| 129 | L8-1 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGT |
| 130 | L8-2 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTC |
| 131 | L8-3 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCA |
| 132 | L8-4 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAA |
| 133 | L8-5 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAG |
| 134 | L9a-1 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCC |
| 135 | L9a-2 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCA |
| 136 | L9a-3 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAA |
| 137 | L9a-4 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAAT |
| 138 | L9a-5 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAATA |
| 139 | L9-1 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAATAGGCAGTAGC |
| 140 | L9-2 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAATAGGCAGTAGCG |
| 141 | L9-3 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAATAGGCAGTAGCGA |
| 142 | L9-4 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAATAGGCAGTAGCGAA |
| 143 | L9-5 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAATAGGCAGTAGCGAAA |
| 144 | L9-6 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAATAGGCAGTAGCGAAAG |
| 145 | L9-7 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAATAGGCAGTAGCGAAAGCTGC |
| 146 | L9-8 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAATAGGCAGTAGCGAAAGCTGCA |
| 147 | L9-9 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAATAGGCAGTAGCGAAAGCTGCA |
| | | A |
| 148 | L9a-6 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAATAG |
| 149 | L9a-7 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAATAGGC |
| 150 | L9a-8 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACT |
| | | CGACGGGAGCTACCCTAACGTCAAGACGAGGGTAAAGAGAG |
| | | AGTCCAATTCTCAAAGCCAATAGGCA |

In some embodiments, a 3′ intron fragment is a fragment having a sequence listed in Table 19. In some embodiments, a construct containing a 3′ intron fragment listed in Table 19 will contain a corresponding 5′ intron fragment as listed in Table 18 (e.g., both representing fragments with the L9a-8 permutation site).

TABLE 20
Non-anabaena permutation site 5’ intron fragment sequences.

| SEQ | | |
|---|---|---|
| ID NO | Intron | Sequence |
| 151 | Azop1 | tgcgccgatgaaggtgtagagactagacggcacccacctaaggcaaacgctatggtgaaggcatagtcca |
| | | gggagtggcgaaagtcacacaaaccggaatccgt |
| 152 | Azop2 | ccgggcgtatggcaacgccgagccaagcttcggcgcctgcgccgatgaaggtgtagagactagacggc |
| | | acccacctaaggcaaacgctatggtgaaggcatagtccagggagtggcgaaagtcacacaaaccggaat |
| | | ccgt |
| 153 | Azop3 | acggcacccacctaaggcaaacgctatggtgaaggcatagtccagggagtggcgaaagtcacacaaacc |
| | | ggaatccgt |
| 154 | Azop4 | acgctatggtgaaggcatagtccagggagtggcgaaagtcacacaaaccggaatccgt |
| 155 | S795p1 | attaaagttatagaattatcagagaatgatatagtccaagccttatggtaacatgagggcacttgaccctggta |
| | | g |
| 156 | Twortp1 | aagatgtaggcaatcctgagctaagctcttagtaataagagaaagtgcaacgactattccgataggaagtag |
| | | ggtcaagtgactcgaaatggggattacccttctagggtagtgatatagtctgaacatatatggaaacatatag |
| | | aaggataggagtaacgaacctattcgtaacataattgaacttttagttat |
| 157 | Twortp2 | taataagagaaagtgcaacgactattccgataggaagtagggtcaagtgactcgaaatggggattacccttc |
| | | tagggtagtgatatagtctgaacatatatggaaacatatagaaggataggagtaacgaacctattcgtaacat |
| | | aattgaacttttagttat |
| 158 | Twortp3 | taggaagtagggtcaagtgactcgaaatggggattacccttctagggtagtgatatagtctgaacatatatgg |
| | | aaacatatagaaggataggagtaacgaacctattcgtaacataattgaacttttagttat |
| 159 | Twortp4 | ctagggtagtgatatagtctgaacatatatggaaacatatagaaggataggagtaacgaacctattcgtaaca |
| | | taattgaacttttagttat |
| 160 | LSUp1 | agttaataaagatgatgaaatagtctgaaccattttgagaaaagtggaaataaaagaaaatcttttatgataac |
| | | ataaattgaacaggctaa |
| 161 | Phip1 | caaagactgatgatatagtccgacactcctagtaataggagaatacagaaaggatgaaatcc |
| 162 | Nostoc | agtcgagggtaaagggagagtccaattctcaaagcctattggcagtagcgaaagctgcgggagaatgaaa |
| | | atccgt |
| 163 | Nostoc | agccgagggtaaagggagagtccaattctcaaagccaataggcagtagcgaaagctgcgggagaatgaa |
| | | aatccgt |
| 164 | Nodularia | agccgagggtaaagggagagtccaattctcaaagccgaaggttattaaaacctggcagcagtgaaagctg |
| | | cgggagaatgaaaatccgt |
| 165 | Pleurocapsa | agctgagggtaaagagagagtccaattctcaaagccagcagatggcagtagcgaaagctgcgggagaat |
| | | gaaaatccgt |
| 166 | Planktothrix | agccgagggtaaagagagagtccaattctcaaagccaattggtagtagcgaaagctacgggagaatgaaa |
| | | atccgt |

In some embodiments, a 5′ intron fragment is a fragment having a sequence listed in Table 20. A construct containing a 5′ intron fragment listed in Table 20 will contain a corresponding 3′ intron fragment in Table 21 (e.g., both representing fragments with the Azop1 intron).

TABLE 21
Non-anabaena permutation site 3’ intron fragment sequences.

| SEQ | | |
|---|---|---|
| ID NO | Intron | Sequence |
| 167 | Azop1 | gcggactcatatttcgatgtgccttgcgccgggaaaccacgcaagggatggtgtcaaattcggcgaaac |
| | | ctaagcgcccgcccgggcgtatggcaacgccgagccaagcttcggcgcc |
| 168 | Azop2 | gcggactcatatttcgatgtgccttgcgccgggaaaccacgcaagggatggtgtcaaattcggcgaaac |
| | | ctaagcgcccgc |
| 169 | Azop3 | gcggactcatatttcgatgtgccttgcgccgggaaaccacgcaagggatggtgtcaaattcggcgaaac |
| | | ctaagcgcccgcccgggcgtatggcaacgccgagccaagcttcggcgcctgcgccgatgaaggtgta |
| | | gagactag |
| 170 | Azop4 | gcggactcatatttcgatgtgccttgcgccgggaaaccacgcaagggatggtgtcaaattcggcgaaac |
| | | ctaagcgcccgcccgggcgtatggcaacgccgagccaagcttcggcgcctgcgccgatgaaggtgta |
| | | gagactagacggcacccacctaaggcaa |
| 171 | S795p1 | aggattagatactacactaagtgtcccccagactggtgacagtctggtgtgcatccagctatatcggtgaa |
| | | accccattggggtaataccgagggaagctatattatatatatattaataaatagccccgtagagactatgta |
| | | ggtaaggagatagaagatgataaaatcaaaatcatc |
| 172 | Twortp1 | actactgaaagcataaataattgtgcctttatacagtaatgtatatcgaaaaatcctctaattcagggaacac |
| | | ctaaacaaact |
| 173 | Twortp2 | actactgaaagcataaataattgtgcctttatacagtaatgtatatcgaaaaatcctctaattcagggaacac |
| | | ctaaacaaactaagatgtaggcaatcctgagctaagctcttag |
| 174 | Twortp3 | actactgaaagcataaataattgtgcctttatacagtaatgtatatcgaaaaatcctctaattcagggaacac |
| | | ctaaacaaactaagatgtaggcaatcctgagctaagctcttagtaataagagaaagtgcaacgactattcc |
| | | ga |
| 175 | Twortp4 | actactgaaagcataaataattgtgcctttatacagtaatgtatatcgaaaaatcctctaattcagggaacac |
| | | ctaaacaaactaagatgtaggcaatcctgagctaagctcttagtaataagagaaagtgcaacgactattcc |
| | | gataggaagtagggtcaagtgactcgaaatggggattaccctt |
| 176 | LSUp1 | cgctagggatttataactgtgagtcctccaatattataaaatgttggtaatatattgggtaaatttcaaagaca |
| | | acttttctccacgtcaggatatagtgtatttgaagcgaaacttattttagcagtgaaaaagcaaataaggac |
| | | gttcaacgactaaaaggtgagtattgctaacaataatccttttttttaatgcccaacatctttattaact |
| 177 | Phip1 | gtgggtgcataaactatttcattgtgcacattaaatctggtgaactcggtgaaaccctaatggggcaatacc |
| | | gagccaagccatagggaggatatatgagaggcaagaagttaattcttgaggccactgagactggctgta |
| | | tcatccctacgtcacacaaacttaatgccgatggttatttcagaaagaaaaccaatggcgtcttagagatgt |
| | | atcacagaacggtgtggaaggagcataacggagacatacctgatggcttcgagatagaccataagtgtc |
| | | gcaatagggcttgctgtaatatagagcatttacagatgcttgagggtacagcccacactgttaagaccaat |
| | | cgtgaacgctacgcagacagaaaggaaacagctagggaatactggctggagactggatgtaccggcc |
| | | tagcactcggtgagaagtttggtgtgtcgttctcttctgcttgtaagtggattagagaatggaaggcgtaga |
| | | gactatccgaaaggagtagggccgagggtgagactccctcgtaacccgaagcgccagacagtcaact |
| 178 | Nostoc | acggacttaagtaattgagccttaaagaagaaattctttaagtggcagctctcaaactcagggaaacctaa |
| | | atctgttcacagacaaggcaatcctgagccaagccgaaagagtcatgagtgctgagtagtgagtaaaat |
| | | aaaagctcacaactcagaggttgtaactctaagctagtcggaaggtgcagagactcgacgggagctac |
| | | cctaacgtaa |
| 179 | Nostoc | acggacttaaactgaattgagccttagagaagaaattctttaagtgtcagctctcaaactcagggaaacct |
| | | aaatctgttgacagacaaggcaatcctgagccaagccgagaactctaagttattcggaaggtgcagaga |
| | | ctcgacgggagctaccctaacgtca |
| 180 | Nodularia | acggacttagaaaactgagccttgatcgagaaatctttcaagtggaagctctcaaattcagggaaacctaa |
| | | atctgtttacagatatggcaatcctgagccaagccgaaacaagtcctgagtgttaaagctcataactcatc |
| | | ggaaggtgcagagactcgacgggagctaccctaacgtta |
| 181 | Pleurocapsa | acggacttaaaaaaattgagccttggcagagaaatctgtcatgcgaacgctctcaaattcagggaaacct |
| | | aagtctggcaacagatatggcaatcctgagccaagccttaatcaaggaaaaaaacatttttaccttttacctt |
| | | gaaaggaaggtgcagagactcaacgggagctaccctaacaggtca |
| 182 | Planktothrix | acggacttaaagataaattgagccttgaggcgagaaatctctcaagtgtaagctgtcaaattcagggaaa |
| | | cctaaatctgtaaattcagacaaggcaatcctgagccaagcctaggggtattagaaatgagggagtttcc |
| | | ccaatctaagatcaatacctaggaaggtgcagagactcgacgggagctaccctaacgtta |

In some embodiments, a 3′ intron fragment is a fragment having a sequence listed in Table 21. A construct containing a 3′ intron fragment listed in Table 21 will contain the corresponding 5′ intron fragment as listed in Table 20 (e.g., both representing fragments with the Azop1 intron).

TABLE 22
Spacer and Anabaena 5’ intron fragment sequences.

| SEQ | | |
|---|---|---|
| ID NO | Spacer | Sequence |
| 183 | T25 L10 | agtatataagaaacaaaccacTAGATGACTTACAACTAATCGGAAGGTGC |
| | | AGAGACTCGACGGGAGCTACCCTAACGTCAAGACGAGGGTAA |
| | | AGAGAGAGTCCAATTCTCAAAGCCAATAGGCAGTAGCGAAAG |
| | | CTGCAAGAGAATGAAAATCCGTggctcgcagc |
| 184 | T25 L20 | ctgaaattatacttatactcaaacaaaccacTAGATGACTTACAACTAATCGGAA |
| | | GGTGCAGAGACTCGACGGGAGCTACCCTAACGTCAAGACGAG |
| | | GGTAAAGAGAGAGTCCAATTCTCAAAGCCAATAGGCAGTAGC |
| | | GAAAGCTGCAAGAGAATGAAAATCCGTggctcgcagc |
| 185 | T25 L30 | ctgaaattatacttatactcagtatatgacaaacaaaccacTAGATGACTTACAACTAA |
| | (180-10) | TCGGAAGGTGCAGAGACTCGACGGGAGCTACCCTAACGTCAA |
| | [Control] | GACGAGGGTAAAGAGAGAGTCCAATTCTCAAAGCCAATAGGC |
| | | AGTAGCGAAAGCTGCAAGAGAATGAAAATCCGTggctcgcagc |
| 186 | T25 L40 | catcaacaatatgaaattatacttatactcagtatatgacaaacaaaccacTAGATGACTTAC |
| | | AACTAATCGGAAGGTGCAGAGACTCGACGGGAGCTACCCTAA |
| | | CGTCAAGACGAGGGTAAAGAGAGAGTCCAATTCTCAAAGCCA |
| | | ATAGGCAGTAGCGAAAGCTGCAAGAGAATGAAAATCCGTggct |
| | | cgcagc |
| 187 | T25 L50 | catcaacaatatgaaactatacttatactcagtatatgaagcattatcgcaaacaaaccacTAGATG |
| | | ACTTACAACTAATCGGAAGGTGCAGAGACTCGACGGGAGCTA |
| | | CCCTAACGTCAAGACGAGGGTAAAGAGAGAGTCCAATTCTCA |
| | | AAGCCAATAGGCAGTAGCGAAAGCTGCAAGAGAATGAAAAT |
| | | CCGTggctcgcagc |
| 188 | T50 L10 | tagcgtcagcaaacaaacaaaTAGATGACTTACAACTAATCGGAAGGTGC |
| | | AGAGACTCGACGGGAGCTACCCTAACGTCAAGACGAGGGTAA |
| | | AGAGAGAGTCCAATTCTCAAAGCCAATAGGCAGTAGCGAAAG |
| | | CTGCAAGAGAATGAAAATCCGTggctcgcagc |
| 189 | T50 L20 | atactcatactagcgtcagcaaacaaacaaaTAGATGACTTACAACTAATCGGA |
| | | AGGTGCAGAGACTCGACGGGAGCTACCCTAACGTCAAGACGA |
| | | GGGTAAAGAGAGAGTCCAATTCTCAAAGCCAATAGGCAGTAG |
| | | CGAAAGCTGCAAGAGAATGAAAATCCGTggctcgcagc |
| 190 | T50 L30 | gtgtgaagctatactcatactagcgtcagcaaacaaacaaaTAGATGACTTACAACTA |
| | | ATCGGAAGGTGCAGAGACTCGACGGGAGCTACCCTAACGTCA |
| | | AGACGAGGGTAAAGAGAGAGTCCAATTCTCAAAGCCAATAGG |
| | | CAGTAGCGAAAGCTGCAAGAGAATGAAAATCCGTggctcgcagc |
| 191 | T50 L40 | cctcacctgagtgtgaagctatactcatactagcgtcagcaaacaaacaaaTAGATGACTTA |
| | | CAACTAATCGGAAGGTGCAGAGACTCGACGGGAGCTACCCTA |
| | | ACGTCAAGACGAGGGTAAAGAGAGAGTCCAATTCTCAAAGCC |
| | | AATAGGCAGTAGCGAAAGCTGCAAGAGAATGAAAATCCGTgg |
| | | ctcgcagc |
| 192 | T50 L50 | ccgaatgatgcctcacctgagtgtgaagctatactcatactagcgtcagcaaacaaacaaaTAGAT |
| | | GACTTACAACTAATCGGAAGGTGCAGAGACTCGACGGGAGCT |
| | | ACCCTAACGTCAAGACGAGGGTAAAGAGAGAGTCCAATTCTC |
| | | AAAGCCAATAGGCAGTAGCGAAAGCTGCAAGAGAATGAAAA |
| | | TCCGTggctcgcagc |
| 193 | T75 L10 | cggtgcgagcaaacaaacaaaTAGATGACTTACAACTAATCGGAAGGTG |
| | | CAGAGACTCGACGGGAGCTACCCTAACGTCAAGACGAGGGTA |
| | | AAGAGAGAGTCCAATTCTCAAAGCCAATAGGCAGTAGCGAAA |
| | | GCTGCAAGAGAATGAAAATCCGTggctcgcagc |
| 194 | T75 L20 | cgctccgacccagtgcgagcaaacaaacaaaTAGATGACTTACAACTAATCGG |
| | | AAGGTGCAGAGACTCGACGGGAGCTACCCTAACGTCAAGACG |
| | | AGGGTAAAGAGAGAGTCCAATTCTCAAAGCCAATAGGCAGTA |
| | | GCGAAAGCTGCAAGAGAATGAAAATCCGTggctcgcagc |
| 195 | T25 L30 | ctgaaattatactAatactcagtatatgacaaacaaaccacTAGATGACTTACAACTAA |
| | 1MM | TCGGAAGGTGCAGAGACTCGACGGGAGCTACCCTAACGTCAA |
| | | GACGAGGGTAAAGAGAGAGTCCAATTCTCAAAGCCAATAGGC |
| | | AGTAGCGAAAGCTGCAAGAGAATGAAAATCCGTggctcgcagc |
| 196 | T25 L30 | ctgaaaAtatactAatactcaCtatatgacaaacaaaccacTAGATGACTTACAACTA |
| | 3MM | ATCGGAAGGTGCAGAGACTCGACGGGAGCTACCCTAACGTCA |
| | | AGACGAGGGTAAAGAGAGAGTCCAATTCTCAAAGCCAATAGG |
| | | CAGTAGCGAAAGCTGCAAGAGAATGAAAATCCGTggctcgcagc |
| 197 | T25 L30 | ctgaTaAtataGtAatactcaCtatatgacaaacaaaccacTAGATGACTTACAACT |
| | 5MM | AATCGGAAGGTGCAGAGACTCGACGGGAGCTACCCTAACGTC |
| | | AAGACGAGGGTAAAGAGAGAGTCCAATTCTCAAAGCCAATAG |
| | | GCAGTAGCGAAAGCTGCAAGAGAATGAAAATCCGTggctcgcagc |
| 198 | T25 L30 | ctgaTaAtaAaGtAatacAcaCtataAgacaaacaaaccacTAGATGACTTACAA |
| | 8MM | CTAATCGGAAGGTGCAGAGACTCGACGGGAGCTACCCTAACG |
| | | TCAAGACGAGGGTAAAGAGAGAGTCCAATTCTCAAAGCCAAT |
| | | AGGCAGTAGCGAAAGCTGCAAGAGAATGAAAATCCGTggctcgc |
| | | agc |
| 199 | T25 L30 | ctgaaattatacttatactctctaagttacaaacaaaccacTAGATGACTTACAACTAAT |
| | OffTarget 10 | CGGAAGGTGCAGAGACTCGACGGGAGCTACCCTAACGTCAAG |
| | | ACGAGGGTAAAGAGAGAGTCCAATTCTCAAAGCCAATAGGCA |
| | | GTAGCGAAAGCTGCAAGAGAATGAAAATCCGTggctcgcagc |
| 200 | T25 L30 | ctgaaattatgtgtgttacAtctaagttacaaacaaaccacTAGATGACTTACAACTAA |
| | OffTarget 20 | TCGGAAGGTGCAGAGACTCGACGGGAGCTACCCTAACGTCAA |
| | | GACGAGGGTAAAGAGAGAGTCCAATTCTCAAAGCCAATAGGC |
| | | AGTAGCGAAAGCTGCAAGAGAATGAAAATCCGTggctcgcagc |
| 201 | T25 L30 | gttgatcggtgtgtgttacAtctaagttacaaacaaaccacTAGATGACTTACAACTAA |
| | OffTarget 30 | TCGGAAGGTGCAGAGACTCGACGGGAGCTACCCTAACGTCAA |
| | | GACGAGGGTAAAGAGAGAGTCCAATTCTCAAAGCCAATAGGC |
| | | AGTAGCGAAAGCTGCAAGAGAATGAAAATCCGTggctcgcagc |
| 202 | T25 L30 I25- | ctgaaattatacttatactcagtatatgacaaacaaaccacTAGATGACTTACAACTAA |
| | 10 | TCGGAAGGTGCAGAGACTCGACGGGAGCTACCCTAACGTCAA |
| | | GACGAGGGTAAAGAGAGAGTCCAATTCTCAAAGCCAATAGGC |
| | | AGTAGCGAAAGCTGCAAGAGAATGAAAATCCGTgattaaacag |
| 203 | T25 L30 I25- | ctgaaattatacttatactcagtatatgacaaacaaaccacTAGATGACTTACAACTAA |
| | 20 | TCGGAAGGTGCAGAGACTCGACGGGAGCTACCCTAACGTCAA |
| | | GACGAGGGTAAAGAGAGAGTCCAATTCTCAAAGCCAATAGGC |
| | | AGTAGCGAAAGCTGCAAGAGAATGAAAATCCGTgattcacaatataaa |
| | | ttacg |
| 204 | T25 L30 I50- | ctgaaattatacttatactcagtatatgacaaacaaaccacTAGATGACTTACAACTAA |
| | 10 | TCGGAAGGTGCAGAGACTCGACGGGAGCTACCCTAACGTCAA |
| | | GACGAGGGTAAAGAGAGAGTCCAATTCTCAAAGCCAATAGGC |
| | | AGTAGCGAAAGCTGCAAGAGAATGAAAATCCGTggatcatagc |
| 205 | T25 L30 I50- | ctgaaattatacttatactcagtatatgacaaacaaaccacTAGATGACTTACAACTAA |
| | 20 | TCGGAAGGTGCAGAGACTCGACGGGAGCTACCCTAACGTCAA |
| | | GACGAGGGTAAAGAGAGAGTCCAATTCTCAAAGCCAATAGGC |
| | | AGTAGCGAAAGCTGCAAGAGAATGAAAATCCGTggatcgcagcataa |
| | | tatccg |
| 206 | T25 L30 I80- | ctgaaattatacttatactcagtatatgacaaacaaaccacTAGATGACTTACAACTAA |
| | 20 | TCGGAAGGTGCAGAGACTCGACGGGAGCTACCCTAACGTCAA |
| | | GACGAGGGTAAAGAGAGAGTCCAATTCTCAAAGCCAATAGGC |
| | | AGTAGCGAAAGCTGCAAGAGAATGAAAATCCGTggctcgcagcgcg |
| | | cctaccg |
| 207 | T25 L30 I80- | ctgaaattatacttatactcagtatatgacaaacaaaccacTAGATGACTTACAACTAA |
| | 20 × 2 | TCGGAAGGTGCAGAGACTCGACGGGAGCTACCCTAACGTCAA |
| | | GACGAGGGTAAAGAGAGAGTCCAATTCTCAAAGCCAATAGGC |
| | | AGTAGCGAAAGCTGCAAGAGAATGAAAATCCGTggctcgcagcgcg |
| | | cctaccgaaagccggcgtcgacgttagcgc |
| 208 | T25 L30 I50- | ctgaaattatacttatactcagtatatgacaaacaaaccacTAGATGACTTACAACTAA |
| | 20 × 2 | TCGGAAGGTGCAGAGACTCGACGGGAGCTACCCTAACGTCAA |
| | | GACGAGGGTAAAGAGAGAGTCCAATTCTCAAAGCCAATAGGC |
| | | AGTAGCGAAAGCTGCAAGAGAATGAAAATCCGTggatcgcagcataa |
| | | tatccgaaacgaggatacaagtgacatgc |
| 209 | T25 L30 I25- | ctgaaattatacttatactcagtatatgacaaacaaaccacTAGATGACTTACAACTAA |
| | 20 × 2 | TCGGAAGGTGCAGAGACTCGACGGGAGCTACCCTAACGTCAA |
| | | GACGAGGGTAAAGAGAGAGTCCAATTCTCAAAGCCAATAGGC |
| | | AGTAGCGAAAGCTGCAAGAGAATGAAAATCCGTgattcacaatctaaa |
| | | ttacgaaacgataaatgataactctaac |
| 210 | T0 L0 | aaacaaaccacTAGATGACTTACAACTAATCGGAAGGTGCAGAGAC |
| | | TCGACGGGAGCTACCCTAACGTCAAGACGAGGGTAAAGAGAG |
| | | AGTCCAATTCTCAAAGCCAATAGGCAGTAGCGAAAGCTGCAA |
| | | GAGAATGAAAATCCGTggctcgcagc |
| 211 | T100 L5 | cgggcaaacaaacaaaTAGATGACTTACAACTAATCGGAAGGTGCAG |
| | | AGACTCGACGGGAGCTACCCTAACGTCAAGACGAGGGTAAAG |
| | | AGAGAGTCCAATTCTCAAAGCCAATAGGCAGTAGCGAAAGCT |
| | | GCAAGAGAATGAAAATCCGTggctcgcagc |
| 212 | T75 L30 | cgctccgacgagcttccggccagtgcgagcaaacaaacaaaTAGATGACTTACAACT |
| | | AATCGGAAGGTGCAGAGACTCGACGGGAGCTACCCTAACGTC |
| | | AAGACGAGGGTAAAGAGAGAGTCCAATTCTCAAAGCCAATAG |
| | | GCAGTAGCGAAAGCTGCAAGAGAATGAAAATCCGTggctcgcagc |
| 213 | T0 L0a | aaacaaaccacGGCAGTAGCGAAAGCTGCAAGAGAATGAAAATCC |
| | | GTggctcgcagc |
| 214 | T25 L10a | agtatataagaaacaaaccacGGCAGTAGCGAAAGCTGCAAGAGAATGA |
| | | AAATCCGTggctcgcagc |
| 215 | T25 L20a | ctgaaattatacttatactcaaacaaaccacGGCAGTAGCGAAAGCTGCAAGAG |
| | | AATGAAAATCCGTggctcgcagc |
| 216 | T25 L30a | ctgaaattatacttatactcagtatatgacaaacaaaccacGGCAGTAGCGAAAGCTGC |
| | (I80-10) | AAGAGAATGAAAATCCGTggctcgcagc |
| | [Control] | |
| 217 | T50 L10a | tagcgtcagcaaacaaacaaaGGCAGTAGCGAAAGCTGCAAGAGAATGA |
| | | AAATCCGTggctcgcagc |
| 218 | T50 L20a | atactcatactagcgtcagcaaacaaacaaaGGCAGTAGCGAAAGCTGCAAGAG |
| | | AATGAAAATCCGTggctcgcagc |
| 219 | T50 L30a | gtgtgaagctatactcatactagcgtcagcaaacaaacaaaGGCAGTAGCGAAAGCTG |
| | | CAAGAGAATGAAAATCCGTggctcgcagc |
| 220 | T75 L10a | cggtgcgagcaaacaaacaaaGGCAGTAGCGAAAGCTGCAAGAGAATGA |
| | | AAATCCGTggctcgcagc |
| 221 | T75 L20a | cgctccgacccagtgcgagcaaacaaacaaaGGCAGTAGCGAAAGCTGCAAGA |
| | | GAATGAAAATCCGTggctcgcagc |
| 222 | T75 L30a | cgctccgacgagcttccggccagtgcgagcaaacaaacaaaGGCAGTAGCGAAAGCT |
| | | GCAAGAGAATGAAAATCCGTggctcgcagc |
| 223 | T0 L0b | aaacaaaccacAAGACGAGGGTAAAGAGAGAGTCCAATTCTCAAA |
| | | GCCAATAGGCAGTAGCGAAAGCTGCAAGAGAATGAAAATCC |
| | | GTggctcgcagc |
| 224 | T25 L10b | agtatataagaaacaaaccacAAGACGAGGGTAAAGAGAGAGTCCAATTC |
| | | TCAAAGCCAATAGGCAGTAGCGAAAGCTGCAAGAGAATGAA |
| | | AATCCGTggctcgcagc |
| 225 | T25 L20b | ctgaaattatacttatactcaaacaaaccacAAGACGAGGGTAAAGAGAGAGTC |
| | | CAATTCTCAAAGCCAATAGGCAGTAGCGAAAGCTGCAAGAGA |
| | | ATGAAAATCCGTggctcgcagc |
| 226 | T25 L30b | ctgaaattatacttatactcagtatatgacaaacaaaccacAAGACGAGGGTAAAGAG |
| | (I80-10) | AGAGTCCAATTCTCAAAGCCAATAGGCAGTAGCGAAAGCTGC |
| | [Control] | AAGAGAATGAAAATCCGTggctcgcagc |
| 227 | T50 L10b | tagcgtcagcaaacaaacaaaAAGACGAGGGTAAAGAGAGAGTCCAATT |
| | | CTCAAAGCCAATAGGCAGTAGCGAAAGCTGCAAGAGAATGA |
| | | AAATCCGTggctcgcagc |
| 228 | T50 L20b | atactcatactagcgtcagcaaacaaacaaaAAGACGAGGGTAAAGAGAGAGT |
| | | CCAATTCTCAAAGCCAATAGGCAGTAGCGAAAGCTGCAAGAG |
| | | AATGAAAATCCGTggctcgcagc |
| 229 | T50 L30b | gtgtgaagctatactcatactagcgtcagcaaacaaacaaaAAGACGAGGGTAAAGA |
| | | GAGAGTCCAATTCTCAAAGCCAATAGGCAGTAGCGAAAGCTG |
| | | CAAGAGAATGAAAATCCGTggctcgcagc |
| 230 | T75 L10b | cggtgcgagcaaacaaacaaaAAGACGAGGGTAAAGAGAGAGTCCAATT |
| | | CTCAAAGCCAATAGGCAGTAGCGAAAGCTGCAAGAGAATGA |
| | | AAATCCGTggctcgcagc |
| 231 | T75 L20b | cgctccgacccagtgcgagcaaacaaacaaaAAGACGAGGGTAAAGAGAGAG |
| | | TCCAATTCTCAAAGCCAATAGGCAGTAGCGAAAGCTGCAAGA |
| | | GAATGAAAATCCGTggctcgcagc |
| 232 | T75 L30b | cgctccgacgagcttccggccagtgcgagcaaacaaacaaaAAGACGAGGGTAAAG |
| | | AGAGAGTCCAATTCTCAAAGCCAATAGGCAGTAGCGAAAGCT |
| | | GCAAGAGAATGAAAATCCGTggctcgcagc |
| 233 | T25 L30 I0-0 | ctgaaattatacttatactcagtatatgacaaacaaaccacTAGATGACTTACAACTAA |
| | | TCGGAAGGTGCAGAGACTCGACGGGAGCTACCCTAACGTCAA |
| | | GACGAGGGTAAAGAGAGAGTCCAATTCTCAAAGCCAATAGGC |
| | | AGTAGCGAAAGCTGCAAGAGAATGAAAATCCGT |
| 234 | T25 L30a I0- | ctgaaattatacttatactcagtatatgacaaacaaaccacGGCAGTAGCGAAAGCTGC |
| | 0 | AAGAGAATGAAAATCCGT |
| 235 | T25 L30a | ctgaaattatacttatactcagtatatgacaaacaaaccacGGCAGTAGCGAAAGCTGC |
| | I25-10 | AAGAGAATGAAAATCCGTgattaaacag |
| 236 | T25 L30a | ctgaaattatacttatactcagtatatgacaaacaaaccacGGCAGTAGCGAAAGCTGC |
| | I25-20 | AAGAGAATGAAAATCCGTgattcacaatataaattacg |
| 237 | T25 L30a | ctgaaattatacttatactcagtatatgacaaacaaaccacGGCAGTAGCGAAAGCTGC |
| | I50-10 | AAGAGAATGAAAATCCGTggatcatagc |
| 238 | T25 L30a | ctgaaattatacttatactcagtatatgacaaacaaaccacGGCAGTAGCGAAAGCTGC |
| | I50-20 | AAGAGAATGAAAATCCGTggatcgcagcataatatccg |
| 239 | T25 L30a | ctgaaattatacttatactcagtatatgacaaacaaaccacGGCAGTAGCGAAAGCTGC |
| | I80-20 | AAGAGAATGAAAATCCGTggctcgcagcgcgcctaccg |
| 240 | T25 L30b I0- | ctgaaattatacttatactcagtatatgacaaacaaaccacAAGACGAGGGTAAAGAG |
| | 0 | AGAGTCCAATTCTCAAAGCCAATAGGCAGTAGCGAAAGCTGC |
| | | AAGAGAATGAAAATCCGT |
| 241 | T25 L30b | ctgaaattatacttatactcagtatatgacaaacaaaccacAAGACGAGGGTAAAGAG |
| | I25-10 | AGAGTCCAATTCTCAAAGCCAATAGGCAGTAGCGAAAGCTGC |
| | | AAGAGAATGAAAATCCGTgattaaacag |
| 242 | T25 L30b | ctgaaattatacttatactcagtatatgacaaacaaaccacAAGACGAGGGTAAAGAG |
| | I25-20 | AGAGTCCAATTCTCAAAGCCAATAGGCAGTAGCGAAAGCTGC |
| | | AAGAGAATGAAAATCCGTgattcacaatataaattacg |
| 243 | T25 L30b | ctgaaattatacttatactcagtatatgacaaacaaaccacAAGACGAGGGTAAAGAG |
| | I50-10 | AGAGTCCAATTCTCAAAGCCAATAGGCAGTAGCGAAAGCTGC |
| | | AAGAGAATGAAAATCCGTggatcatagc |
| 244 | T25 L30b | ctgaaattatacttatactcagtatatgacaaacaaaccacAAGACGAGGGTAAAGAG |
| | I50-20 | AGAGTCCAATTCTCAAAGCCAATAGGCAGTAGCGAAAGCTGC |
| | | AAGAGAATGAAAATCCGTggatcgcagcataatatccg |
| 245 | T25 L30b | ctgaaattatacttatactcagtatatgacaaacaaaccacAAGACGAGGGTAAAGAG |
| | I80-20 | AGAGTCCAATTCTCAAAGCCAATAGGCAGTAGCGAAAGCTGC |
| | | AAGAGAATGAAAATCCGTggctcgcagcgcgcctaccg |

In some embodiments, a spacer and 5′ intron fragment are spacers and fragments having sequences as listed in Table 22.

TABLE 23
Spacer and Anabaena 3′ intron fragment sequences.

| SEQ | | |
|---|---|---|
| ID NO | Spacer | Sequence |
| 246 | T25 L10 | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAcacaaacacaacttatatact |
| 247 | T25 L20 | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAcacaaacacaagagtataagtataatttcag |
| 248 | T25 L30 | gctgcgagccACGGACTTAAATAATTGAGCCTTAAAGAAGAAATTC |
| | (180-10) | TTTAAGTGGATGCTCTCAAACTCAGGGAAACCTAAATCTAGTT |
| | [Control] | ATAGACAAGGCAATCCTGAGCCAAGCCGAAGTAGTAATTAGT |
| | | AAGTTAACAAcacaaacacaagtcatatactgagtataagtataatttcag |
| 249 | T25 L40 | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAcacaaacacaagtcatatactgagtataagtataatttcatattgttgatg |
| 250 | T25 L50 | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAcacaaacacaagcgataatgcttcatatactgagtataagtatagtttcatattg |
| | | ttgatg |
| 251 | T50 L10 | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAaacaaaaacaagctgacgcta |
| 252 | T50 L20 | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAaacaaaaacaagctgacgctagtatgagtat |
| 253 | T50 L30 | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAaacaaaaacaagctgacgctagtatgagtatagcttcacac |
| 254 | T50 L40 | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAaacaaaaacaagctgacgctagtatgagtatagcttcacactcaggtgagg |
| 255 | T50 L50 | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAaacaaaaacaagctgacgctagtatgagtatagcttcacactcaggtgaggc |
| | | atcattcgg |
| 256 | T75 L10 | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAaacaaaaacaagctcgcaccg |
| 257 | T75 L20 | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAaacaaaaacaagctcgcactgggtcggagcg |
| 258 | T25 L30 | gctgcgagccACGGACTTAAATAATTGAGCCTTAAAGAAGAAATTC |
| | 1MM | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAcacaaacacaagtcatatactgagtataagtataatttcag |
| 259 | T25 L30 | gctgcgagccACGGACTTAAATAATTGAGCCTTAAAGAAGAAATTC |
| | 3MM | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAcacaaacacaagtcatatactgagtataagtataatttcag |
| 260 | T25 L30 | gctgcgagccACGGACTTAAATAATTGAGCCTTAAAGAAGAAATTC |
| | 5MM | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAcacaaacacaagtcatatactgagtataagtataatttcag |
| 261 | T25 L30 | gctgcgagccACGGACTTAAATAATTGAGCCTTAAAGAAGAAATTC |
| | 8MM | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAcacaaacacaagtcatatactgagtataagtataatttcag |
| 262 | T25 L30 | gctgcgagccACGGACTTAAATAATTGAGCCTTAAAGAAGAAATTC |
| | OffTarget 10 | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAcacaaacacaagtaacttagagagtataagtataatttcag |
| 263 | T25 L30 | gctgcgagccACGGACTTAAATAATTGAGCCTTAAAGAAGAAATTC |
| | OffTarget 20 | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAcacaaacacaagtaacttagaTgtaacacacataatttcag |
| 264 | T25 L30 | gctgcgagccACGGACTTAAATAATTGAGCCTTAAAGAAGAAATTC |
| | OffTarget 30 | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAcacaaacacaagtaacttagaTgtaacacacaccgatcaac |
| 265 | T25 L30 I25- | ctgtttaatcACGGACTTAAATAATTGAGCCTTAAAGAAGAAATTCT |
| | 10 | TTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAcacaaacacaagtcatatactgagtataagtataatttcag |
| 266 | T25 L30 I25- | cgtaatttatattgtgaatcACGGACTTAAATAATTGAGCCTTAAAGAAGA |
| | 20 | AATTCTTTAAGTGGATGCTCTCAAACTCAGGGAAACCTAAATC |
| | | TAGTTATAGACAAGGCAATCCTGAGCCAAGCCGAAGTAGTAA |
| | | TTAGTAAGTTAACAAcacaaacacaagtcatatactgagtataagtataatttcag |
| 267 | T25 L30 I50- | gctatgatccACGGACTTAAATAATTGAGCCTTAAAGAAGAAATTC |
| | 10 | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAcacaaacacaagtcatatactgagtataagtataatttcag |
| 268 | T25 L30 I50- | cggatattatgctgcgatccACGGACTTAAATAATTGAGCCTTAAAGAAG |
| | 20 | AAATTCTTTAAGTGGATGCTCTCAAACTCAGGGAAACCTAAA |
| | | TCTAGTTATAGACAAGGCAATCCTGAGCCAAGCCGAAGTAGT |
| | | AATTAGTAAGTTAACAAcacaaacacaagtcatatactgagtataagtataatttcag |
| 269 | T25 L30 I80- | cggtaggcgcgctgcgagccACGGACTTAAATAATTGAGCCTTAAAGAA |
| | 20 | GAAATTCTTTAAGTGGATGCTCTCAAACTCAGGGAAACCTAA |
| | | ATCTAGTTATAGACAAGGCAATCCTGAGCCAAGCCGAAGTAG |
| | | TAATTAGTAAGTTAACAAcacaaacacaagtcatatactgagtataagtataatttcag |
| 270 | T25 L30 I80- | gcgctaacgtcgacgccggcaaacggtaggcgcgctgcgagccACGGACTTAAATAA |
| | 20 × 2 | TTGAGCCTTAAAGAAGAAATTCTTTAAGTGGATGCTCTCAAAC |
| | | TCAGGGAAACCTAAATCTAGTTATAGACAAGGCAATCCTGAG |
| | | CCAAGCCGAAGTAGTAATTAGTAAGTTAACAAcacaaacacaagtcat |
| | | atactgagtataagtataatttcag |
| 271 | T25 L30 I50- | gcatgtcacttgtatcctcgaaacggatattatgctgcgatccACGGACTTAAATAATTG |
| | 20 × 2 | AGCCTTAAAGAAGAAATTCTTTAAGTGGATGCTCTCAAACTC |
| | | AGGGAAACCTAAATCTAGTTATAGACAAGGCAATCCTGAGCC |
| | | AAGCCGAAGTAGTAATTAGTAAGTTAACAAcacaaacacaagtcatatac |
| | | tgagtataagtataatttcag |
| 272 | T25 L30 I25- | gttagagttatcatttatcgaaacgtaatttagattgtgaatcACGGACTTAAATAATTGA |
| | 20 × 2 | GCCTTAAAGAAGAAATTCTTTAAGTGGATGCTCTCAAACTCA |
| | | GGGAAACCTAAATCTAGTTATAGACAAGGCAATCCTGAGCCA |
| | | AGCCGAAGTAGTAATTAGTAAGTTAACAAcacaaacacaagtcatatactg |
| | | agtataagtataatttcag |
| 273 | TO L0 | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAcacaaacacaa |
| 274 | T100 L5 | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAaacaaaaacaagcccg |
| 275 | T75 L30 | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAAaacaaaaacaagctcgcactggccggaagctcgtcggagcg |
| 276 | T0 L0a | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCAAGACGAGGGTAAAGA |
| | | GAGAGTCCAATTCTCAAAGCCAATAcacaaacacaa |
| 277 | T25 L10a | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCAAGACGAGGGTAAAGA |
| | | GAGAGTCCAATTCTCAAAGCCAATAcacaaacacaacttatatact |
| 278 | T25 L20a | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCAAGACGAGGGTAAAGA |
| | | GAGAGTCCAATTCTCAAAGCCAATAcacaaacacaagagtataagtataatttc |
| | | ag |
| 279 | T25 L30a | gctgcgagccACGGACTTAAATAATTGAGCCTTAAAGAAGAAATTC |
| | (180-10) | TTTAAGTGGATGCTCTCAAACTCAGGGAAACCTAAATCTAGTT |
| | [Control] | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCAAGACGAGGGTAAAGA |
| | | GAGAGTCCAATTCTCAAAGCCAATAcacaaacacaagtcatatactgagtataa |
| | | gtataatttcag |
| 280 | T50 L10a | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCAAGACGAGGGTAAAGA |
| | | GAGAGTCCAATTCTCAAAGCCAATAaacaaaaacaagctgacgcta |
| 281 | T50 L20a | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCAAGACGAGGGTAAAGA |
| | | GAGAGTCCAATTCTCAAAGCCAATAaacaaaaacaagctgacgctagtatga |
| | | gtat |
| 282 | T50 L30a | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCAAGACGAGGGTAAAGA |
| | | GAGAGTCCAATTCTCAAAGCCAATAaacaaaaacaagctgacgctagtatga |
| | | gtatagcttcacac |
| 283 | T75 L10a | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCAAGACGAGGGTAAAGA |
| | | GAGAGTCCAATTCTCAAAGCCAATAaacaaaaacaagctcgcaccg |
| 284 | T75 L20a | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCAAGACGAGGGTAAAGA |
| | | GAGAGTCCAATTCTCAAAGCCAATAaacaaaaacaagctcgcactgggtcgg |
| | | agcg |
| 285 | T75 L30a | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCAAGACGAGGGTAAAGA |
| | | GAGAGTCCAATTCTCAAAGCCAATAaacaaaaacaagctcgcactggccgga |
| | | agctcgtcggagcg |
| 286 | T0 L0b | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCcacaaacacaa |
| 287 | T25 L10b | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCcacaaacacaacttatatact |
| 288 | T25 L20b | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCcacaaacacaagagtataagtataatt |
| | | tcag |
| 289 | T25 L30b | gctgcgagccACGGACTTAAATAATTGAGCCTTAAAGAAGAAATTC |
| | (180-10) | TTTAAGTGGATGCTCTCAAACTCAGGGAAACCTAAATCTAGTT |
| | [Control] | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCcacaaacacaagtcatatactgagtat |
| | | aagtataatttcag |
| 290 | T50 L10b | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCaacaaaaacaagctgacgcta |
| 291 | T50 L20b | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCaacaaaaacaagctgacgctagtatg |
| | | agtat |
| 292 | T50 L30b | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCaacaaaaacaagctgacgctagtatg |
| | | agtatagcttcacac |
| 293 | T75 L10b | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCaacaaaaacaagctcgcaccg |
| 294 | T75 L20b | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCaacaaaaacaagctcgcactgggtcg |
| | | gagcg |
| 295 | T75 L30b | gctgcgagccACGGACTTAAATAATTGAGCCTTAAAGAAGAAATTC |
| | | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCaacaaaaacaagctcgcactggccg |
| | | gaagctcgtcggagcg |
| 296 | T25 L30 I0-0 | ACGGACTTAAATAATTGAGCCTTAAAGAAGAAATTCTTTAAG |
| | | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAAcacaaacacaagtcatatactgagtataagtataatttcag |
| 297 | T25 L30aI0- | ACGGACTTAAATAATTGAGCCTTAAAGAAGAAATTCTTTAAG |
| | 0 | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACTC |
| | | GACGGGAGCTACCCTAACGTCAAGACGAGGGTAAAGAGAGA |
| | | GTCCAATTCTCAAAGCCAATAcacaaacacaagtcatatactgagtataagtataat |
| | | ttcag |
| 298 | T25 L30a | ctgtttaatcACGGACTTAAATAATTGAGCCTTAAAGAAGAAATTCT |
| | I25-10 | TTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCAAGACGAGGGTAAAGA |
| | | GAGAGTCCAATTCTCAAAGCCAATAcacaaacacaagtcatatactgagtataa |
| | | gtataatttcag |
| 299 | T25 L30a | cgtaatttatattgtgaatcACGGACTTAAATAATTGAGCCTTAAAGAAGA |
| | I25-20 | AATTCTTTAAGTGGATGCTCTCAAACTCAGGGAAACCTAAATC |
| | | TAGTTATAGACAAGGCAATCCTGAGCCAAGCCGAAGTAGTAA |
| | | TTAGTAAGTTAACAATAGATGACTTACAACTAATCGGAAGGT |
| | | GCAGAGACTCGACGGGAGCTACCCTAACGTCAAGACGAGGGT |
| | | AAAGAGAGAGTCCAATTCTCAAAGCCAATAcacaaacacaagtcatatac |
| | | tgagtataagtataatttcag |
| 300 | T25 L30a | gctatgatccACGGACTTAAATAATTGAGCCTTAAAGAAGAAATTC |
| | I50-10 | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCAAGACGAGGGTAAAGA |
| | | GAGAGTCCAATTCTCAAAGCCAATAcacaaacacaagtcatatactgagtataa |
| | | gtataatttcag |
| 301 | T25 L30a | cggatattatgctgcgatccACGGACTTAAATAATTGAGCCTTAAAGAAG |
| | I50-20 | AAATTCTTTAAGTGGATGCTCTCAAACTCAGGGAAACCTAAA |
| | | TCTAGTTATAGACAAGGCAATCCTGAGCCAAGCCGAAGTAGT |
| | | AATTAGTAAGTTAACAATAGATGACTTACAACTAATCGGAAG |
| | | GTGCAGAGACTCGACGGGAGCTACCCTAACGTCAAGACGAGG |
| | | GTAAAGAGAGAGTCCAATTCTCAAAGCCAATAcacaaacacaagtcat |
| | | atactgagtataagtataatttcag |
| 302 | T25 L30a | cggtaggcgcgctgcgagccACGGACTTAAATAATTGAGCCTTAAAGAA |
| | I80-20 | GAAATTCTTTAAGTGGATGCTCTCAAACTCAGGGAAACCTAA |
| | | ATCTAGTTATAGACAAGGCAATCCTGAGCCAAGCCGAAGTAG |
| | | TAATTAGTAAGTTAACAATAGATGACTTACAACTAATCGGAA |
| | | GGTGCAGAGACTCGACGGGAGCTACCCTAACGTCAAGACGAG |
| | | GGTAAAGAGAGAGTCCAATTCTCAAAGCCAATAcacaaacacaagtc |
| | | atatactgagtataagtataatttcag |
| 303 | T25 L30b I0- | ACGGACTTAAATAATTGAGCCTTAAAGAAGAAATTCTTTAAG |
| | 0 | TGGATGCTCTCAAACTCAGGGAAACCTAAATCTAGTTATAGA |
| | | CAAGGCAATCCTGAGCCAAGCCGAAGTAGTAATTAGTAAGTT |
| | | AACAATAGATGACTTACAACTAATCGGAAGGTGCAGAGACTC |
| | | GACGGGAGCTACCCTAACGTCcacaaacacaagtcatatactgagtataagtataat |
| | | ttcag |
| 304 | T25 L30b | ctgtttaatcACGGACTTAAATAATTGAGCCTTAAAGAAGAAATTCT |
| | I25-10 | TTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCcacaaacacaagtcatatactgagtat |
| | | aagtataatttcag |
| 305 | T25 L30b | cgtaatttatattgtgaatcACGGACTTAAATAATTGAGCCTTAAAGAAGA |
| | I25-20 | AATTCTTTAAGTGGATGCTCTCAAACTCAGGGAAACCTAAATC |
| | | TAGTTATAGACAAGGCAATCCTGAGCCAAGCCGAAGTAGTAA |
| | | TTAGTAAGTTAACAATAGATGACTTACAACTAATCGGAAGGT |
| | | GCAGAGACTCGACGGGAGCTACCCTAACGTCcacaaacacaagtcatat |
| | | actgagtataagtataatttcag |
| 306 | T25 L30b | gctatgatccACGGACTTAAATAATTGAGCCTTAAAGAAGAAATTC |
| | I50-10 | TTTAAGTGGATGCTCTCAAACTCAGGGAAACCTAAATCTAGTT |
| | | ATAGACAAGGCAATCCTGAGCCAAGCCGAAGTAGTAATTAGT |
| | | AAGTTAACAATAGATGACTTACAACTAATCGGAAGGTGCAGA |
| | | GACTCGACGGGAGCTACCCTAACGTCcacaaacacaagtcatatactgagtat |
| | | aagtataatttcag |
| 307 | T25 L30b | cggatattatgctgcgatccACGGACTTAAATAATTGAGCCTTAAAGAAG |
| | I50-20 | AAATTCTTTAAGTGGATGCTCTCAAACTCAGGGAAACCTAAA |
| | | TCTAGTTATAGACAAGGCAATCCTGAGCCAAGCCGAAGTAGT |
| | | AATTAGTAAGTTAACAATAGATGACTTACAACTAATCGGAAG |
| | | GTGCAGAGACTCGACGGGAGCTACCCTAACGTCcacaaacacaagtc |
| | | atatactgagtataagtataatttcag |
| 308 | T25 L30b | cggtaggcgcgctgcgagccACGGACTTAAATAATTGAGCCTTAAAGAA |
| | I80-20 | GAAATTCTTTAAGTGGATGCTCTCAAACTCAGGGAAACCTAA |
| | | ATCTAGTTATAGACAAGGCAATCCTGAGCCAAGCCGAAGTAG |
| | | TAATTAGTAAGTTAACAATAGATGACTTACAACTAATCGGAA |
| | | GGTGCAGAGACTCGACGGGAGCTACCCTAACGTCcacaaacacaagt |
| | | catatactgagtataagtataatttcag |

In some embodiments, a spacer and 3′ intron fragment is a spacer and intron fragment having sequences as listed in Table 23.

TABLE 24
CAR sequences

| SEQ | | |
|---|---|---|
| ID NO | CAR | Sequence |
| 309 | FMC63-4- | ATGCTGCTGCTGGTCACATCTCTGCTGCTGTGCGAGCTGCCCC |
| | 1BB | ATCCTGCCTTTCTGCTGATCCCCGACATCCAGATGACCCAGAC |
| | | CACAAGCAGCCTGTCTGCCAGCCTGGGCGATAGAGTGACCAT |
| | | CAGCTGTAGAGCCAGCCAGGACATCAGCAAGTACCTGAACTG |
| | | GTATCAGCAAAAGCCCGACGGCACCGTGAAGCTGCTGATCTA |
| | | CCACACCAGCAGACTGCACAGCGGCGTGCCAAGCAGATTTTC |
| | | TGGCAGCGGCTCTGGCACCGACTACAGCCTGACAATCAGCAA |
| | | CCTGGAACAAGAGGATATCGCTACCTACTTCTGCCAGCAAGG |
| | | CAACACCCTGCCTTACACCTTTGGCGGAGGCACCAAGCTGGA |
| | | AATCACCGGCTCTACAAGCGGCAGCGGCAAACCTGGATCTGG |
| | | CGAGGGATCTACCAAGGGCGAAGTGAAACTGCAAGAGTCTG |
| | | GCCCTGGACTGGTGGCCCCATCTCAGTCTCTGAGCGTGACCTG |
| | | TACAGTCAGCGGAGTGTCCCTGCCTGATTACGGCGTGTCCTG |
| | | GATCAGACAGCCTCCTCGGAAAGGCCTGGAATGGCTGGGAGT |
| | | GATCTGGGGCAGCGAGACAACCTACTACAACAGCGCCCTGAA |
| | | GTCCCGGCTGACCATCATCAAGGACAACTCCAAGAGCCAGGT |
| | | GTTCCTGAAGATGAACAGCCTGCAGACCGACGACACCGCCAT |
| | | CTACTATTGCGCCAAGCACTACTACTACGGCGGCAGCTACGC |
| | | CATGGATTATTGGGGCCAGGGCACCAGCGTGACCGTTTCTTCT |
| | | GCCGCCGCTATCGAAGTGATGTACCCTCCTCCTTACCTGGACA |
| | | ACGAGAAGTCCAACGGCACCATCATCCACGTGAAGGGCAAG |
| | | CACCTGTGTCCTTCTCCACTGTTCCCCGGACCTAGCAAGCCTT |
| | | TCTGGGTGCTCGTTGTTGTTGGCGGCGTGCTGGCCTGTTACAG |
| | | CCTGCTGGTTACCGTGGCCTTCATCATCTTTTGGGTCAAGAGA |
| | | GGCCGGAAGAAACTTCTTTATATATTCAAGCAGCCCTTTATGC |
| | | GACCCGTTCAGACTACCCAAGAGGAAGATGGATGCAGTTGCC |
| | | GCTTTCCAGAAGAGGAGGAGGGCGGGTGCGAACTGtaa |
| 310 | FMC63-CD28 | ATGCTGCTGCTGGTCACATCTCTGCTGCTGTGCGAGCTGCCCC |
| | | ATCCTGCCTTTCTGCTGATCCCCGACATCCAGATGACCCAGAC |
| | | CACAAGCAGCCTGTCTGCCAGCCTGGGCGATAGAGTGACCAT |
| | | CAGCTGTAGAGCCAGCCAGGACATCAGCAAGTACCTGAACTG |
| | | GTATCAGCAAAAGCCCGACGGCACCGTGAAGCTGCTGATCTA |
| | | CCACACCAGCAGACTGCACAGCGGCGTGCCAAGCAGATTTTC |
| | | TGGCAGCGGCTCTGGCACCGACTACAGCCTGACAATCAGCAA |
| | | CCTGGAACAAGAGGATATCGCTACCTACTTCTGCCAGCAAGG |
| | | CAACACCCTGCCTTACACCTTTGGCGGAGGCACCAAGCTGGA |
| | | AATCACCGGCTCTACAAGCGGCAGCGGCAAACCTGGATCTGG |
| | | CGAGGGATCTACCAAGGGCGAAGTGAAACTGCAAGAGTCTG |
| | | GCCCTGGACTGGTGGCCCCATCTCAGTCTCTGAGCGTGACCTG |
| | | TACAGTCAGCGGAGTGTCCCTGCCTGATTACGGCGTGTCCTG |
| | | GATCAGACAGCCTCCTCGGAAAGGCCTGGAATGGCTGGGAGT |
| | | GATCTGGGGCAGCGAGACAACCTACTACAACAGCGCCCTGAA |
| | | GTCCCGGCTGACCATCATCAAGGACAACTCCAAGAGCCAGGT |
| | | GTTCCTGAAGATGAACAGCCTGCAGACCGACGACACCGCCAT |
| | | CTACTATTGCGCCAAGCACTACTACTACGGCGGCAGCTACGC |
| | | CATGGATTATTGGGGCCAGGGCACCAGCGTGACCGTTTCTTCT |
| | | GCCGCCGCTATCGAAGTGATGTACCCTCCTCCTTACCTGGACA |
| | | ACGAGAAGTCCAACGGCACCATCATCCACGTGAAGGGCAAG |
| | | CACCTGTGTCCTTCTCCACTGTTCCCCGGACCTAGCAAGCCTT |
| | | TCTGGGTGCTCGTTGTTGTTGGCGGCGTGCTGGCCTGTTACAG |
| | | CCTGCTGGTTACCGTGGCCTTCATCATCTTTTGGGTCCGAAGC |
| | | AAGCGGAGCCGGCTGCTGCACTCCGACTACATGAACATGACC |
| | | CCTAGACGGCCCGGACCAACCAGAAAGCACTACCAGCCTTAC |
| | | GCTCCTCCTAGAGACTTCGCCGCCTACCGGTCCtaa |
| 311 | FMC63- | ATGCTGCTGCTGGTCACATCTCTGCTGCTGTGCGAGCTGCCCC |
| | CD28-zeta | ATCCTGCCTTTCTGCTGATCCCCGACATCCAGATGACCCAGAC |
| | | CACAAGCAGCCTGTCTGCCAGCCTGGGCGATAGAGTGACCAT |
| | | CAGCTGTAGAGCCAGCCAGGACATCAGCAAGTACCTGAACTG |
| | | GTATCAGCAAAAGCCCGACGGCACCGTGAAGCTGCTGATCTA |
| | | CCACACCAGCAGACTGCACAGCGGCGTGCCAAGCAGATTTTC |
| | | TGGCAGCGGCTCTGGCACCGACTACAGCCTGACAATCAGCAA |
| | | CCTGGAACAAGAGGATATCGCTACCTACTTCTGCCAGCAAGG |
| | | CAACACCCTGCCTTACACCTTTGGCGGAGGCACCAAGCTGGA |
| | | AATCACCGGCTCTACAAGCGGCAGCGGCAAACCTGGATCTGG |
| | | CGAGGGATCTACCAAGGGCGAAGTGAAACTGCAAGAGTCTG |
| | | GCCCTGGACTGGTGGCCCCATCTCAGTCTCTGAGCGTGACCTG |
| | | TACAGTCAGCGGAGTGTCCCTGCCTGATTACGGCGTGTCCTG |
| | | GATCAGACAGCCTCCTCGGAAAGGCCTGGAATGGCTGGGAGT |
| | | GATCTGGGGCAGCGAGACAACCTACTACAACAGCGCCCTGAA |
| | | GTCCCGGCTGACCATCATCAAGGACAACTCCAAGAGCCAGGT |
| | | GTTCCTGAAGATGAACAGCCTGCAGACCGACGACACCGCCAT |
| | | CTACTATTGCGCCAAGCACTACTACTACGGCGGCAGCTACGC |
| | | CATGGATTATTGGGGCCAGGGCACCAGCGTGACCGTTTCTTCT |
| | | GCCGCCGCTATCGAAGTGATGTACCCTCCTCCTTACCTGGACA |
| | | ACGAGAAGTCCAACGGCACCATCATCCACGTGAAGGGCAAG |
| | | CACCTGTGTCCTTCTCCACTGTTCCCCGGACCTAGCAAGCCTT |
| | | TCTGGGTGCTCGTTGTTGTTGGCGGCGTGCTGGCCTGTTACAG |
| | | CCTGCTGGTTACCGTGGCCTTCATCATCTTTTGGGTCCGAAGC |
| | | AAGCGGAGCCGGCTGCTGCACTCCGACTACATGAACATGACC |
| | | CCTAGACGGCCCGGACCAACCAGAAAGCACTACCAGCCTTAC |
| | | GCTCCTCCTAGAGACTTCGCCGCCTACCGGTCCAGAGTGAAG |
| | | TTCAGCAGATCCGCCGATGCTCCCGCCTATCAGCAGGGCCAA |
| | | AACCAGCTGTACAACGAGCTGAACCTGGGGAGAAGAGAAGA |
| | | GTACGACGTGCTGGACAAGCGGAGAGGCAGAGATCCTGAAA |
| | | TGGGCGGCAAGCCCAGACGGAAGAATCCTCAAGAGGGCCTG |
| | | TATAATGAGCTGCAGAAAGACAAGATGGCCGAGGCCTACAG |
| | | CGAGATCGGAATGAAGGGCGAGCGCAGAAGAGGCAAGGGAC |
| | | ACGATGGACTGTACCAGGGACTGAGCACCGCCACCAAGGATA |
| | | CCTATGACGCCCTGCACATGCAGGCCCTGCCTCCAAGAtaa |
| 312 | FMC63-zeta | ATGCTGCTGCTGGTCACATCTCTGCTGCTGTGCGAGCTGCCCC |
| | | ATCCTGCCTTTCTGCTGATCCCCGACATCCAGATGACCCAGAC |
| | | CACAAGCAGCCTGTCTGCCAGCCTGGGCGATAGAGTGACCAT |
| | | CAGCTGTAGAGCCAGCCAGGACATCAGCAAGTACCTGAACTG |
| | | GTATCAGCAAAAGCCCGACGGCACCGTGAAGCTGCTGATCTA |
| | | CCACACCAGCAGACTGCACAGCGGCGTGCCAAGCAGATTTTC |
| | | TGGCAGCGGCTCTGGCACCGACTACAGCCTGACAATCAGCAA |
| | | CCTGGAACAAGAGGATATCGCTACCTACTTCTGCCAGCAAGG |
| | | CAACACCCTGCCTTACACCTTTGGCGGAGGCACCAAGCTGGA |
| | | AATCACCGGCTCTACAAGCGGCAGCGGCAAACCTGGATCTGG |
| | | CGAGGGATCTACCAAGGGCGAAGTGAAACTGCAAGAGTCTG |
| | | GCCCTGGACTGGTGGCCCCATCTCAGTCTCTGAGCGTGACCTG |
| | | TACAGTCAGCGGAGTGTCCCTGCCTGATTACGGCGTGTCCTG |
| | | GATCAGACAGCCTCCTCGGAAAGGCCTGGAATGGCTGGGAGT |
| | | GATCTGGGGCAGCGAGACAACCTACTACAACAGCGCCCTGAA |
| | | GTCCCGGCTGACCATCATCAAGGACAACTCCAAGAGCCAGGT |
| | | GTTCCTGAAGATGAACAGCCTGCAGACCGACGACACCGCCAT |
| | | CTACTATTGCGCCAAGCACTACTACTACGGCGGCAGCTACGC |
| | | CATGGATTATTGGGGCCAGGGCACCAGCGTGACCGTTTCTTCT |
| | | GCCGCCGCTATCGAAGTGATGTACCCTCCTCCTTACCTGGACA |
| | | ACGAGAAGTCCAACGGCACCATCATCCACGTGAAGGGCAAG |
| | | CACCTGTGTCCTTCTCCACTGTTCCCCGGACCTAGCAAGCCTT |
| | | TCTGGGTGCTCGTTGTTGTTGGCGGCGTGCTGGCCTGTTACAG |
| | | CCTGCTGGTTACCGTGGCCTTCATCATCTTTTGGGTCAGAGTG |
| | | AAGTTCAGCAGATCCGCCGATGCTCCCGCCTATCAGCAGGGC |
| | | CAAAACCAGCTGTACAACGAGCTGAACCTGGGGAGAAGAGA |
| | | AGAGTACGACGTGCTGGACAAGCGGAGAGGCAGAGATCCTG |
| | | AAATGGGCGGCAAGCCCAGACGGAAGAATCCTCAAGAGGGC |
| | | CTGTATAATGAGCTGCAGAAAGACAAGATGGCCGAGGCCTAC |
| | | AGCGAGATCGGAATGAAGGGCGAGCGCAGAAGAGGCAAGGG |
| | | ACACGATGGACTGTACCAGGGACTGAGCACCGCCACCAAGG |
| | | ATACCTATGACGCCCTGCACATGCAGGCCCTGCCTCCAAGAtaa |
| 313 | CircKymriah - | ATGGCTCTCCCGGTCACAGCCCTTCTCCTGCCCCTGGCACTCT |
| | Q388 | TGCTGCATGCGGCACGACCCGACATCCAGATGACCCAGACCA |
| | | CAAGCAGCCTGTCTGCCAGCCTGGGCGATAGAGTGACCATCA |
| | | GCTGTAGAGCCAGCCAGGACATCAGCAAGTACCTGAACTGGT |
| | | ATCAGCAAAAGCCCGACGGCACCGTGAAGCTGCTGATCTACC |
| | | ACACCAGCAGACTGCACAGCGGCGTGCCAAGCAGATTTTCTG |
| | | GCAGCGGCTCTGGCACCGACTACAGCCTGACAATCAGCAACC |
| | | TGGAACAAGAGGATATCGCTACCTACTTCTGCCAGCAAGGCA |
| | | ACACCCTGCCTTACACCTTTGGCGGAGGCACCAAGCTGGAAA |
| | | TCACCGGTGGAGGTGGTTCTGGCGGAGGGGGATCTGGTGGAG |
| | | GCGGTTCAGAAGTGAAACTGCAAGAGTCTGGCCCTGGACTGG |
| | | TGGCCCCATCTCAGTCTCTGAGCGTGACCTGTACAGTCAGCG |
| | | GAGTGTCCCTGCCTGATTACGGCGTGTCCTGGATCAGACAGC |
| | | CTCCTCGGAAAGGCCTGGAATGGCTGGGAGTGATCTGGGGCA |
| | | GCGAGACAACCTACTACAACAGCGCCCTGAAGTCCCGGCTGA |
| | | CCATCATCAAGGACAACTCCAAGAGCCAGGTGTTCCTGAAGA |
| | | TGAACAGCCTGCAGACCGACGACACCGCCATCTACTATTGCG |
| | | CCAAGCACTACTACTACGGCGGCAGCTACGCCATGGATTATT |
| | | GGGGCCAGGGCACCAGCGTGACCGTTTCTTCTACCACAACGC |
| | | CCGCCCCGCGACCGCCTACTCCCGCTCCCACAATTGCATCACA |
| | | ACCCCTGTCTTTGAGACCCGAAGCTTGTCGACCAGCTGCCGGT |
| | | GGCGCGGTTCACACGCGGGGGCTCGATTTCGCCTGTGATATA |
| | | TATATATGGGCCCCATTGGCTGGAACATGCGGAGTATTGCTTC |
| | | TGAGCCTGGTGATTACCCTCTACTGTAAGAGAGGCCGGAAGA |
| | | AACTTCTTTATATATTCAAGCAGCCCTTTATGCGACCCGTTCA |
| | | GACTACCCAAGAGGAAGATGGATGCAGTTGCCGCTTTCCAGA |
| | | AGAGGAGGAGGGCGGGTGCGAACTGAGAGTGAAGTTCAGCA |
| | | GATCCGCCGATGCTCCCGCCTATCAGCAGGGCCAAAACCAGC |
| | | TGTACAACGAGCTGAACCTGGGGAGAAGAGAAGAGTACGAC |
| | | GTGCTGGACAAGCGGAGAGGCAGAGATCCTGAAATGGGCGG |
| | | CAAGCCCAGACGGAAGAATCCTCAAGAGGGCCTGTATAATGA |
| | | GCTGCAGAAAGACAAGATGGCCGAGGCCTACAGCGAGATCG |
| | | GAATGAAGGGCGAGCGCAGAAGAGGCAAGGGACACGATGGA |
| | | CTGTACCAGGGACTGAGCACCGCCACCAAGGATACCTATGAC |
| | | GCCCTGCACATGCAGGCCCTGCCTCCAAGAtaa |
| 314 | CircKymriah - | ATGGCTCTCCCGGTCACAGCCCTTCTCCTGCCCCTGGCACTCT |
| | K388 | TGCTGCATGCGGCACGACCCGACATCCAGATGACCCAGACCA |
| | | CAAGCAGCCTGTCTGCCAGCCTGGGCGATAGAGTGACCATCA |
| | | GCTGTAGAGCCAGCCAGGACATCAGCAAGTACCTGAACTGGT |
| | | ATCAGCAAAAGCCCGACGGCACCGTGAAGCTGCTGATCTACC |
| | | ACACCAGCAGACTGCACAGCGGCGTGCCAAGCAGATTTTCTG |
| | | GCAGCGGCTCTGGCACCGACTACAGCCTGACAATCAGCAACC |
| | | TGGAACAAGAGGATATCGCTACCTACTTCTGCCAGCAAGGCA |
| | | ACACCCTGCCTTACACCTTTGGCGGAGGCACCAAGCTGGAAA |
| | | TCACCGGTGGAGGTGGTTCTGGCGGAGGGGGATCTGGTGGAG |
| | | GCGGTTCAGAAGTGAAACTGCAAGAGTCTGGCCCTGGACTGG |
| | | TGGCCCCATCTCAGTCTCTGAGCGTGACCTGTACAGTCAGCG |
| | | GAGTGTCCCTGCCTGATTACGGCGTGTCCTGGATCAGACAGC |
| | | CTCCTCGGAAAGGCCTGGAATGGCTGGGAGTGATCTGGGGCA |
| | | GCGAGACAACCTACTACAACAGCGCCCTGAAGTCCCGGCTGA |
| | | CCATCATCAAGGACAACTCCAAGAGCCAGGTGTTCCTGAAGA |
| | | TGAACAGCCTGCAGACCGACGACACCGCCATCTACTATTGCG |
| | | CCAAGCACTACTACTACGGCGGCAGCTACGCCATGGATTATT |
| | | GGGGCCAGGGCACCAGCGTGACCGTTTCTTCTACCACAACGC |
| | | CCGCCCCGCGACCGCCTACTCCCGCTCCCACAATTGCATCACA |
| | | ACCCCTGTCTTTGAGACCCGAAGCTTGTCGACCAGCTGCCGGT |
| | | GGCGCGGTTCACACGCGGGGGCTCGATTTCGCCTGTGATATA |
| | | TATATATGGGCCCCATTGGCTGGAACATGCGGAGTATTGCTTC |
| | | TGAGCCTGGTGATTACCCTCTACTGTAAGAGAGGCCGGAAGA |
| | | AACTTCTTTATATATTCAAGCAGCCCTTTATGCGACCCGTTCA |
| | | GACTACCCAAGAGGAAGATGGATGCAGTTGCCGCTTTCCAGA |
| | | AGAGGAGGAGGGCGGGTGCGAACTGAGAGTGAAGTTCAGCA |
| | | GATCCGCCGATGCTCCCGCCTATAAGCAGGGCCAAAACCAGC |
| | | TGTACAACGAGCTGAACCTGGGGAGAAGAGAAGAGTACGAC |
| | | GTGCTGGACAAGCGGAGAGGCAGAGATCCTGAAATGGGCGG |
| | | CAAGCCCAGACGGAAGAATCCTCAAGAGGGCCTGTATAATGA |
| | | GCTGCAGAAAGACAAGATGGCCGAGGCCTACAGCGAGATCG |
| | | GAATGAAGGGCGAGCGCAGAAGAGGCAAGGGACACGATGGA |
| | | CTGTACCAGGGACTGAGCACCGCCACCAAGGATACCTATGAC |
| | | GCCCTGCACATGCAGGCCCTGCCTCCAAGAtaa |
| 315 | CircM971 - | ATGCTGCTGCTGGTCACATCTCTGCTGCTGTGCGAGCTGCCCC |
| | CD22 | ATCCTGCCTTTCTGCTGATCCCCCAGGTTCAACTCCAGCAGTC |
| | | TGGTCCCGGCCTCGTTAAACCAAGCCAGACTTTGTCTCTTACC |
| | | TGTGCTATCAGTGGCGATAGCGTGTCTAGTAATTCAGCCGCAT |
| | | GGAACTGGATCCGACAATCACCGAGTAGGGGACTTGAATGGC |
| | | TGGGTAGAACCTATTACCGGTCCAAATGGTACAATGACTATG |
| | | CAGTGTCTGTAAAAAGCAGGATCACGATCAACCCTGATACGT |
| | | CTAAAAACCAGTTTTCTCTGCAACTTAATAGTGTGACCCCTGA |
| | | AGACACCGCTGTGTATTACTGTGCACGGGAGGTTACCGGTGA |
| | | TCTTGAAGATGCTTTTGATATATGGGGCCAAGGTACGATGGT |
| | | CACGGTGTCTAGTgggggaggcggcagcGACATACAGATGACGCAG |
| | | AGCCCATCCAGTCTCTCCGCGTCTGTTGGTGACAGAGTGACTA |
| | | TTACATGTAGGGCGTCTCAGACCATTTGGTCTTACCTCAATTG |
| | | GTATCAACAGCGACCAGGCAAAGCACCGAACTTGCTCATTTA |
| | | CGCTGCCAGCTCACTCCAAAGTGGTGTGCCGTCCAGATTTAGT |
| | | GGTAGGGGCAGTGGCACTGATTTCACTCTGACTATTTCAAGTC |
| | | TTCAAGCTGAGGATTTTGCCACATACTACTGCCAGCAAAGTT |
| | | ACTCAATACCTCAGACTTTTGGACAGGGGACAAAATTGGAGA |
| | | TTAAAtccggaACCACAACGCCCGCCCCGCGACCGCCTACTCCC |
| | | GCTCCCACAATTGCATCACAACCCCTGTCTTTGAGACCCGAA |
| | | GCTTGTCGACCAGCTGCCGGTGGCGCGGTTCACACGCGGGGG |
| | | CTCGATTTCGCCTGTGATATATATATATGGGCCCCATTGGCTG |
| | | GAACATGCGGAGTATTGCTTCTGAGCCTGGTGATTACCCTCTA |
| | | CTGTAAGAGAGGCCGGAAGAAACTTCTTTATATATTCAAGCA |
| | | GCCCTTTATGCGACCCGTTCAGACTACCCAAGAGGAAGATGG |
| | | ATGCAGTTGCCGCTTTCCAGAAGAGGAGGAGGGCGGGTGCGA |
| | | ACTGAGAGTGAAGTTCAGCAGATCCGCCGATGCTCCCGCCTA |
| | | TAAGCAGGGCCAAAACCAGCTGTACAACGAGCTGAACCTGG |
| | | GGAGAAGAGAAGAGTACGACGTGCTGGACAAGCGGAGAGGC |
| | | AGAGATCCTGAAATGGGCGGCAAGCCCAGACGGAAGAATCC |
| | | TCAAGAGGGCCTGTATAATGAGCTGCAGAAAGACAAGATGG |
| | | CCGAGGCCTACAGCGAGATCGGAATGAAGGGCGAGCGCAGA |
| | | AGAGGCAAGGGACACGATGGACTGTACCAGGGACTGAGCAC |
| | | CGCCACCAAGGATACCTATGACGCCCTGCACATGCAGGCCCT |
| | | GCCTCCAAGAtaa |
| 316 | CircCD19_22 | ATGCTGCTGCTGGTCACATCTCTGCTGCTGTGCGAGCTGCCCC |
| | Bispecific 29 | ATCCTGCCTTTCTGCTGATCCCCGACATCCAGATGACCCAGAC |
| | | CACAAGCAGCCTGTCTGCCAGCCTGGGCGATAGAGTGACCAT |
| | | CAGCTGTAGAGCCAGCCAGGACATCAGCAAGTACCTGAACTG |
| | | GTATCAGCAAAAGCCCGACGGCACCGTGAAGCTGCTGATCTA |
| | | CCACACCAGCAGACTGCACAGCGGCGTGCCAAGCAGATTTTC |
| | | TGGCAGCGGCTCTGGCACCGACTACAGCCTGACAATCAGCAA |
| | | CCTGGAACAAGAGGATATCGCTACCTACTTCTGCCAGCAAGG |
| | | CAACACCCTGCCTTACACCTTTGGCGGAGGCACCAAGCTGGA |
| | | AATCACCggcggcggaggatccCAGGTTCAACTCCAGCAGTCTGGTC |
| | | CCGGCCTCGTTAAACCAAGCCAGACTTTGTCTCTTACCTGTGC |
| | | TATCAGTGGCGATAGCGTGTCTAGTAATTCAGCCGCATGGAA |
| | | CTGGATCCGACAATCACCGAGTAGGGGACTTGAATGGCTGGG |
| | | TAGAACCTATTACCGGTCCAAATGGTACAATGACTATGCAGT |
| | | GTCTGTAAAAAGCAGGATCACGATCAACCCTGATACGTCTAA |
| | | AAACCAGTTTTCTCTGCAACTTAATAGTGTGACCCCTGAAGAC |
| | | ACCGCTGTGTATTACTGTGCACGGGAGGTTACCGGTGATCTTG |
| | | AAGATGCTTTTGATATATGGGGCCAAGGTACGATGGTCACGG |
| | | TGTCTAGTGGCTCTACAAGCGGCAGCGGCAAACCTGGATCTG |
| | | GCGAGGGATCTACCAAGGGCGACATACAGATGACGCAGAGC |
| | | CCATCCAGTCTCTCCGCGTCTGTTGGTGACAGAGTGACTATTA |
| | | CATGTAGGGCGTCTCAGACCATTTGGTCTTACCTCAATTGGTA |
| | | TCAACAGCGACCAGGCAAAGCACCGAACTTGCTCATTTACGC |
| | | TGCCAGCTCACTCCAAAGTGGTGTGCCGTCCAGATTTAGTGGT |
| | | AGGGGCAGTGGCACTGATTTCACTCTGACTATTTCAAGTCTTC |
| | | AAGCTGAGGATTTTGCCACATACTACTGCCAGCAAAGTTACT |
| | | CAATACCTCAGACTTTTGGACAGGGGACAAAATTGGAGATTA |
| | | AAgggggaggcggcagcGAAGTGAAACTGCAAGAGTCTGGCCCTGG |
| | | ACTGGTGGCCCCATCTCAGTCTCTGAGCGTGACCTGTACAGTC |
| | | AGCGGAGTGTCCCTGCCTGATTACGGCGTGTCCTGGATCAGA |
| | | CAGCCTCCTCGGAAAGGCCTGGAATGGCTGGGAGTGATCTGG |
| | | GGCAGCGAGACAACCTACTACAACAGCGCCCTGAAGTCCCGG |
| | | CTGACCATCATCAAGGACAACTCCAAGAGCCAGGTGTTCCTG |
| | | AAGATGAACAGCCTGCAGACCGACGACACCGCCATCTACTAT |
| | | TGCGCCAAGCACTACTACTACGGCGGCAGCTACGCCATGGAT |
| | | TATTGGGGCCAGGGCACCAGCGTGACCGTTTCTTCTtccggaACC |
| | | ACAACGCCCGCCCCGCGACCGCCTACTCCCGCTCCCACAATT |
| | | GCATCACAACCCCTGTCTTTGAGACCCGAAGCTTGTCGACCA |
| | | GCTGCCGGTGGCGCGGTTCACACGCGGGGGCTCGATTTCGCC |
| | | TGTGATATATATATATGGGCCCCATTGGCTGGAACATGCGGA |
| | | GTATTGCTTCTGAGCCTGGTGATTACCCTCTACTGTAAGAGAG |
| | | GCCGGAAGAAACTTCTTTATATATTCAAGCAGCCCTTTATGCG |
| | | ACCCGTTCAGACTACCCAAGAGGAAGATGGATGCAGTTGCCG |
| | | CTTTCCAGAAGAGGAGGAGGGCGGGTGCGAACTGAGAGTGA |
| | | AGTTCAGCAGATCCGCCGATGCTCCCGCCTATAAGCAGGGCC |
| | | AAAACCAGCTGTACAACGAGCTGAACCTGGGGAGAAGAGAA |
| | | GAGTACGACGTGCTGGACAAGCGGAGAGGCAGAGATCCTGA |
| | | AATGGGCGGCAAGCCCAGACGGAAGAATCCTCAAGAGGGCC |
| | | TGTATAATGAGCTGCAGAAAGACAAGATGGCCGAGGCCTACA |
| | | GCGAGATCGGAATGAAGGGCGAGCGCAGAAGAGGCAAGGGA |
| | | CACGATGGACTGTACCAGGGACTGAGCACCGCCACCAAGGAT |
| | | ACCTATGACGCCCTGCACATGCAGGCCCTGCCTCCAAGAtaa |
| 317 | CircCD19_22 | ATGCTGCTGCTGGTCACATCTCTGCTGCTGTGCGAGCTGCCCC |
| | Bispecific 30 | ATCCTGCCTTTCTGCTGATCCCCCAGGTTCAACTCCAGCAGTC |
| | | TGGTCCCGGCCTCGTTAAACCAAGCCAGACTTTGTCTCTTACC |
| | | TGTGCTATCAGTGGCGATAGCGTGTCTAGTAATTCAGCCGCAT |
| | | GGAACTGGATCCGACAATCACCGAGTAGGGGACTTGAATGGC |
| | | TGGGTAGAACCTATTACCGGTCCAAATGGTACAATGACTATG |
| | | CAGTGTCTGTAAAAAGCAGGATCACGATCAACCCTGATACGT |
| | | CTAAAAACCAGTTTTCTCTGCAACTTAATAGTGTGACCCCTGA |
| | | AGACACCGCTGTGTATTACTGTGCACGGGAGGTTACCGGTGA |
| | | TCTTGAAGATGCTTTTGATATATGGGGCCAAGGTACGATGGT |
| | | CACGGTGTCTAGTgggggaggcggcagcGACATACAGATGACGCAG |
| | | AGCCCATCCAGTCTCTCCGCGTCTGTTGGTGACAGAGTGACTA |
| | | TTACATGTAGGGCGTCTCAGACCATTTGGTCTTACCTCAATTG |
| | | GTATCAACAGCGACCAGGCAAAGCACCGAACTTGCTCATTTA |
| | | CGCTGCCAGCTCACTCCAAAGTGGTGTGCCGTCCAGATTTAGT |
| | | GGTAGGGGCAGTGGCACTGATTTCACTCTGACTATTTCAAGTC |
| | | TTCAAGCTGAGGATTTTGCCACATACTACTGCCAGCAAAGTT |
| | | ACTCAATACCTCAGACTTTTGGACAGGGGACAAAATTGGAGA |
| | | TTAAAGGGGGAGGCGGATCCGGCGGTGGTGGCTCCGGCGGTG |
| | | GTGGTTCTGGAGGCGGCGGAAGCGGTGGGGGTGGTAGCGAC |
| | | ATCCAGATGACCCAGACCACAAGCAGCCTGTCTGCCAGCCTG |
| | | GGCGATAGAGTGACCATCAGCTGTAGAGCCAGCCAGGACATC |
| | | AGCAAGTACCTGAACTGGTATCAGCAAAAGCCCGACGGCACC |
| | | GTGAAGCTGCTGATCTACCACACCAGCAGACTGCACAGCGGC |
| | | GTGCCAAGCAGATTTTCTGGCAGCGGCTCTGGCACCGACTAC |
| | | AGCCTGACAATCAGCAACCTGGAACAAGAGGATATCGCTACC |
| | | TACTTCTGCCAGCAAGGCAACACCCTGCCTTACACCTTTGGCG |
| | | GAGGCACCAAGCTGGAAATCACCGGCTCTACAAGCGGCAGC |
| | | GGCAAACCTGGATCTGGCGAGGGATCTACCAAGGGCGAAGT |
| | | GAAACTGCAAGAGTCTGGCCCTGGACTGGTGGCCCCATCTCA |
| | | GTCTCTGAGCGTGACCTGTACAGTCAGCGGAGTGTCCCTGCCT |
| | | GATTACGGCGTGTCCTGGATCAGACAGCCTCCTCGGAAAGGC |
| | | CTGGAATGGCTGGGAGTGATCTGGGGCAGCGAGACAACCTAC |
| | | TACAACAGCGCCCTGAAGTCCCGGCTGACCATCATCAAGGAC |
| | | AACTCCAAGAGCCAGGTGTTCCTGAAGATGAACAGCCTGCAG |
| | | ACCGACGACACCGCCATCTACTATTGCGCCAAGCACTACTAC |
| | | TACGGCGGCAGCTACGCCATGGATTATTGGGGCCAGGGCACC |
| | | AGCGTGACCGTTTCTTCTtccggaACCACAACGCCCGCCCCGCGA |
| | | CCGCCTACTCCCGCTCCCACAATTGCATCACAACCCCTGTCTT |
| | | TGAGACCCGAAGCTTGTCGACCAGCTGCCGGTGGCGCGGTTC |
| | | ACACGCGGGGGCTCGATTTCGCCTGTGATATATATATATGGG |
| | | CCCCATTGGCTGGAACATGCGGAGTATTGCTTCTGAGCCTGGT |
| | | GATTACCCTCTACTGTAAGAGAGGCCGGAAGAAACTTCTTTA |
| | | TATATTCAAGCAGCCCTTTATGCGACCCGTTCAGACTACCCAA |
| | | GAGGAAGATGGATGCAGTTGCCGCTTTCCAGAAGAGGAGGA |
| | | GGGCGGGTGCGAACTGAGAGTGAAGTTCAGCAGATCCGCCG |
| | | ATGCTCCCGCCTATAAGCAGGGCCAAAACCAGCTGTACAACG |
| | | AGCTGAACCTGGGGAGAAGAGAAGAGTACGACGTGCTGGAC |
| | | AAGCGGAGAGGCAGAGATCCTGAAATGGGCGGCAAGCCCAG |
| | | ACGGAAGAATCCTCAAGAGGGCCTGTATAATGAGCTGCAGAA |
| | | AGACAAGATGGCCGAGGCCTACAGCGAGATCGGAATGAAGG |
| | | GCGAGCGCAGAAGAGGCAAGGGACACGATGGACTGTACCAG |
| | | GGACTGAGCACCGCCACCAAGGATACCTATGACGCCCTGCAC |
| | | ATGCAGGCCCTGCCTCCAAGAtaa |

In some embodiments, a CAR is encoded by a nucleotide sequence as listed in Table 24.

TABLE 25
CAR domain sequences.

| SEQ | | |
|---|---|---|
| ID NO | Protein | Sequence |
| 318 | 4-1BB | KRGRKKLLYIFKQPFMRPVQTTQEEDGCSCRFPEEEEGGCEL |
| 319 | CD3ζ | RVKFSRSADAPAYKQGQNQLYNELNLGRREEYDVLDKRRGRDPE |
| | intracellular | MGGKPRRKNPQEGLYNELQKDKMAEAYSEIGMKGERRRGKGHD |
| | domain | GLYQGLSTATKDTYDALHMQALPPR |
| 320 | CD28 | QVQLVQSGAEVEKPGASVKVSCKASGYTFTDYYMHWVRQAPGQ |
| | intracellular | GLEWMGWINPNSGGTNYAQKFQGRVTMTRDTSISTAYMELSRLR |
| | signaling | SDDTAVYYCASGWDFDYWGQGTLVTVSSGGGGSGGGGSGGGGS |
| | domain | GGGGSDIVMTQSPSSLSASVGDRVTITCRASQSIRYYLSWYQQKP |
| | | GKAPKLLIYTASILQNGVPSRFSGSGSGTDFTLTISSLQPEDFATYY |
| | | CLQTYTTPDFGPGTKVEIK |
| 321 | FMC63 VH | EVKLQESGPGLVAPSQSLSVTCTVSGVSLPDYGVSWIRQPPRKGLE |
| | | WLGVIWGSETTYYNSALKSRLTIIKDNSKSQVFLKMNSLQTDDTA |
| | | IYYCAKHYYYGGSYAMDYWGQGTSVTVSS |
| 322 | FMC63 VL | DIQMTQTTSSLSASLGDRVTISCRASQDISKYLNWYQQKPDGTVK |
| | | LLIYHTSRLHSGVPSRFSGSGSGTDYSLTISNLEQEDIATYFCQQGN |
| | | TLPYTFGGGTKLEIT |

In some embodiments, a CAR domain encoded by an inventive polynucleotide has a sequence as listed in Table 25.

TABLE 26
PD-1 or PD-L1 sequences.

| SEQ | | |
|---|---|---|
| ID NO | Description | Sequence |
| 323 | Pembrolizumab heavy | QVQLVQSGVEVKKPGASVKVSCKASGYTFTNYYMYWV |
| | chain | RQAPGQGLEWMGGINPSNGGTNFNEKFKNRVTLTTDSST |
| | | TTAYMELKSLQFDDTAVYYCARRDYRFDMGFDYWGQG |
| | | TTVTVSSASTKGPSVFPLAPCSRSTSESTAALGCLVKDYFP |
| | | EPVTVSWNSGALTSGVHTFPAVLQSSGLYSLSSVVTVPSS |
| | | SLGTKTYTCNVDHKPSNTKVDKRVESKYGPPCPPCPAPE |
| | | FLGGPSVFLFPPKPKDTLMISRTPEVTCVVVDVSQEDPEV |
| | | QFNWYVDGVEVHNAKTKPREEQFNSTYRVVSVLTVLHQ |
| | | DWLNGKEYKCKVSNKGLPSSIEKTISKAKGQPREPQVYT |
| | | LPPSQEEMTKNQVSLTCLVKGFYPSDIAVEWESNGQPEN |
| | | NYKTTPPVLDSDGSFFLYSRLTVDKSRWQEGNVFSCSVM |
| | | HEALHNHYTQKSLSLSLGK |
| 324 | Pembrolizumab light | EIVLTQSPATLSLSPGERATLSCRASKGVSTSGYSYLHWY |
| | chain | QQKPGQAPRLLIYLASYLESGVPARFSGSGSGTDFTLTISS |
| | | LEPEDFAVYYCQHSRDLPLTFGGGTKVEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 325 | Nivolumab heavy | QVQLVESGGGVVQPGRSLRLDCKASGITFSNSGMHWVR |
| | chain | QAPGKGLEWVAVIWYDGSKRYYADSVKGRFTISRDNSK |
| | | NTLFLQMNSLRAEDTAVYYCATNDDYWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFLGGPSVF |
| | | LFPPKPKDTLMISRTPEVTCVVVDVSQEDPEVQFNWYVD |
| | | GVEVHNAKTKPREEQFNSTYRVVSVLTVLHQDWLNGKE |
| | | YKCKVSNKGLPSSIEKTISKAKGQPREPQVYTLPPSQEEM |
| | | TKNQVSLTCLVKGFYPSDIAVEWESNGQPENNYKTTPPV |
| | | LDSDGSFFLYSRLTVDKSRWQEGNVFSCSVMHEALHNH |
| | | YTQKSLSLSLGK |
| 326 | Nivolumab light chain | EIVLTQSPATLSLSPGERATLSCRASQSVSSYLAWYQQKP |
| | | GQAPRLLIYDASNRATGIPARFSGSGSGTDFTLTISSLEPE |
| | | DFAVYYCQQSSNWPRTFGQGTKVEIKRTVAAPSVFIFPPS |
| | | DEQLKSGTASVVCLLNNFYPREAKVQWKVDNALQSGNS |
| | | QESVTEQDSKDSTYSLSSTLTLSKADYEKHKVYACEVTH |
| | | QGLSSPVTKSFNRGEC |
| 327 | Atezolizumab heavy | EVQLVESGGGLVQPGGSLRLSCAASGFTFSDSWIHWVRQ |
| | chain | APGKGLEWVAWISPYGGSTYYADSVKGRFTISADTSKNT |
| | | AYLQMNSLRAEDTAVYYCARRHWPGGFDYWGQGTLVT |
| | | VSSASTKGPSVFPLAPSSKSTSGGTAALGCLVKDYFPEPV |
| | | TVSWNSGALTSGVHTFPAVLQSSGLYSLSSVVTVPSSSLG |
| | | TQTYICNVNHKPSNTKVDKKVEPKSCDKTHTCPPCPAPE |
| | | LLGGPSVFLFPPKPKDTLMISRTPEVTCVVVDVSHEDPEV |
| | | KFNWYVDGVEVHNAKTKPREEQYASTYRVVSVLTVLHQ |
| | | DWLNGKEYKCKVSNKALPAPIEKTISKAKGQPREPQVYT |
| | | LPPSREEMTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGK |
| 328 | Atezolizumab light | DIQMTQSPSSLSASVGDRVTITCRASQDVSTAVAWYQQK |
| | chain | PGKAPKLLIYSASFLYSGVPSRFSGSGSGTDFTLTISSLQPE |
| | | DFATYYCQQYLYHPATFGQGTKVEIKRTVAAPSVFIFPPS |
| | | DEQLKSGTASVVCLLNNFYPREAKVQWKVDNALQSGNS |
| | | QESVTEQDSKDSTYSLSSTLTLSKADYEKHKVYACEVTH |
| | | QGLSSPVTKSFNRGEC |
| 329 | Avelumab heavy chain | EVQLLESGGGLVQPGGSLRLSCAASGFTFSSYIMMWVRQ |
| | | APGKGLEWVSSIYPSGGITFYADTVKGRFTISRDNSKNTL |
| | | YLQMNSLRAEDTAVYYCARIKLGTVTTVDYWGQGTLVT |
| | | VSSASTKGPSVFPLAPSSKSTSGGTAALGCLVKDYFPEPV |
| | | TVSWNSGALTSGVHTFPAVLQSSGLYSLSSVVTVPSSSLG |
| | | TQTYICNVNHKPSNTKVDKKVEPKSCDKTHTCPPCPAPE |
| | | LLGGPSVFLFPPKPKDTLMISRTPEVTCVVVDVSHEDPEV |
| | | KFNWYVDGVEVHNAKTKPREEQYNSTYRVVSVLTVLHQ |
| | | DWLNGKEYKCKVSNKALPAPIEKTISKAKGQPREPQVYT |
| | | LPPSRDELTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| 330 | Avelumab light chain | QSALTQPASVSGSPGQSITISCTGTSSDVGGYNYVSWYQQ |
| | | HPGKAPKLMIYDVSNRPSGVSNRFSGSKSGNTASLTISGL |
| | | QAEDEADYYCSSYTSSSTRVFGTGTKVTVLGQPKANPTV |
| | | TLFPPSSEELQANKATLVCLISDFYPGAVTVAWKADGSPV |
| | | KAGVETTKPSKQSNNKYAASSYLSLTPEQWKSHRSYSCQ |
| | | VTHEGSTVEKTVAPTECS |
| 331 | Durvalumab heavy | EVQLVESGGGLVQPGGSLRLSCAASGFTFSRYWMSWVR |
| | chain | QAPGKGLEWVANIKQDGSEKYYVDSVKGRFTISRDNAK |
| | | NSLYLQMNSLRAEDTAVYYCAREGGWFGELAFDYWGQ |
| | | GTLVTVSSASTKGPSVFPLAPSSKSTSGGTAALGCLVKDY |
| | | FPEPVTVSWNSGALTSGVHTFPAVLQSSGLYSLSSVVTVP |
| | | SSSLGTQTYICNVNHKPSNTKVDKRVEPKSCDKTHTCPPC |
| | | PAPEFEGGPSVFLFPPKPKDTLMISRTPEVTCVVVDVSHE |
| | | DPEVKFNWYVDGVEVHNAKTKPREEQYNSTYRVVSVLT |
| | | VLHQDWLNGKEYKCKVSNKALPASIEKTISKAKGQPREP |
| | | QVYTLPPSREEMTKNQVSLTCLVKGFYPSDIAVEWESNG |
| | | QPENNYKTTPPVLDSDGSFFLYSKLTVDKSRWQQGNVFS |
| | | CSVMHEALHNHYTQKSLSLSPGK |
| 332 | Durvalumab light | EIVLTQSPGTLSLSPGERATLSCRASQRVSSSYLAWYQQK |
| | chain | PGQAPRLLIYDASSRATGIPDRFSGSGSGTDFTLTISRLEPE |
| | | DFAVYYCQQYGSLPWTFGQGTKVEIKRTVAAPSVFIFPPS |
| | | DEQLKSGTASVVCLLNNFYPREAKVQWKVDNALQSGNS |
| | | QESVTEQDSKDSTYSLSSTLTLSKADYEKHKVYACEVTH |
| | | QGLSSPVTKSFNRGEC |

In some embodiments, a cleavage site separating expression sequences encoded by an inventive polynucleotide has a sequence listed in Table 26.

TABLE 27
Cytokine sequences.

| SEQ | | |
|---|---|---|
| ID NO | Cytokine | Sequence |
| 333 | IL-2 | APTSSSTKKTQLQLEHLLLDLQMILNGINNYKNPKLTRMLTFKFY |
| | | MPKKATELKHLQCLEEELKPLEEVLNLAQSKNFHLRPRDLISNINV |
| | | IVLELKGSETTFMCEYADETATIVEFLNRWITFCQSIISTLT |
| 334 | IL-12A | RNLPVATPDPGMFPCLHHSQNLLRAVSNMLQKARQTLEFYPCTSE |
| | | EIDHEDITKDKTSTVEACLPLELTKNESCLNSRETSFITNGSCLASR |
| | | KTSFMMALCLSSIYEDLKMYQVEFKTMNAKLLMDPKRQIFLDQN |
| | | MLAVIDELMQALNFNSETVPQKSSLEEPDFYKTKIKLCILLHAFRIR |
| | | AVTIDRVMSYLNAS |
| 335 | IL-12B | IWELKKDVYVVELDWYPDAPGEMVVLTCDTPEEDGITWTLDQSS |
| | | EVLGSGKTLTIQVKEFGDAGQYTCHKGGEVLSHSLLLLHKKEDGI |
| | | WSTDILKDQKEPKNKTFLRCEAKNYSGRFTCWWLTTISTDLTFSV |
| | | KSSRGSSDPQGVTCGAATLSAERVRGDNKEYEYSVECQEDSACPA |
| | | AEESLPIEVMVDAVHKLKYENYTSSFFIRDIIKPDPPKNLQLKPLKN |
| | | SRQVEVSWEYPDTWSTPHSYFSLTFCVQVQGKSKREKKDRVFTD |
| | | KTSATVICRKNASISVRAQDRYYSSSWSEWASVPCS |
| 336 | IL-7 | DCDIEGKDGKQYESVLMVSIDQLLDSMKEIGSNCLNNEFNFFKRHI |
| | | CDANKEGMFLFRAARKLRQFLKMNSTGDFDLHLLKVSEGTTILLN |
| | | CTGQVKGRKPAALGEAQPTKSLEENKSLKEQKKLNDLCFLKRLL |
| | | QEIKTCWNKILMGTKEH |
| 337 | IL-10 | SPGQGTQSENSCTHFPGNLPNMLRDLRDAFSRVKTFFQMKDQLD |
| | | NLLLKESLLEDFKGYLGCQALSEMIQFYLEEVMPQAENQDPDIKA |
| | | HVNSLGENLKTLRLRLRRCHRFLPCENKSKAVEQVKNAFNKLQE |
| | | KGIYKAMSEFDIFINYIEAYMTMKIRN |
| 338 | IL-15 | NWVNVISDLKKIEDLIQSMHIDATLYTESDVHPSCKVTAMKCFLL |
| | | ELQVISLESGDASIHDTVENLIILANNSLSSNGNVTESGCKECEELE |
| | | EKNIKEFLQSFVHIVQMFINTS |
| 339 | IL-18 | YFGKLESKLSVIRNLNDQVLFIDQGNRPLFEDMTDSDCRDNAPRTI |
| | | FIISMYKDSQPRGMAVTISVKCEKISTLSCENKIISFKEMNPPDNIKD |
| | | TKSDIIFFQRSVPGHDNKMQFESSSYEGYFLACEKERDLFKLILKK |
| | | EDELGDRSIMFTVQNED |
| 340 | IL-27beta | RKGPPAALTLPRVQCRASRYPIAVDCSWTLPPAPNSTSPVSFIATY |
| | | RLGMAARGHSWPCLQQTPTSTSCTITDVQLFSMAPYVLNVTAVH |
| | | PWGSSSSFVPFITEHIIKPDPPEGVRLSPLAERQLQVQWEPPGSWPF |
| | | PEIFSLKYWIRYKRQGAARFHRVGPIEATSFILRAVRPRARYYVQV |
| | | AAQDLTDYGELSDWSLPATATMSLGK |
| 341 | IFNgamma | QDPYVKEAENLKKYFNAGHSDVADNGTLFLGILKNWKEESDRKI |
| | | MQSQIVSFYFKLFKNFKDDQSIQKSVETIKEDMNVKFFNSNKKKR |
| | | DDFEKLTNYSVTDLNVQRKAIHELIQVMAELSPAAKTGKRKRSQ |
| | | MLFRG |
| 342 | TGFbeta1 | ALDTNYCFSSTEKNCCVRQLYIDFRKDLGWKWIHEPKGYHANFC |
| | | LGPCPYIWSLDTQYSKVLALYNQHNPGASAAPCCVPQALEPLPIV |
| | | YYVGRKPKVEQLSNMIVRSCKCS |

In some embodiments, a cytokine encoded by an inventive polynucleotide has a sequence as listed in Table 27.

TABLE 28
Transcription factor sequences.

| SEQ | Transcription | |
|---|---|---|
| ID NO | factor | Sequence |
| 343 | FOXP3 | MPNPRPGKPSAPSLALGPSPGASPSWRAAPKASDLLGARGPGGT |
| | | FQGRDLRGGAHASSSSLNPMPPSQLQLPTLPLVMVAPSGARLGP |
| | | LPHLQALLQDRPHFMHQLSTVDAHARTPVLQVHPLESPAMISLT |
| | | PPTTATGVFSLKARPGLPPGINVASLEWVSREPALLCTFPNPSAPR |
| | | KDSTLSAVPQSSYPLLANGVCKWPGCEKVFEEPEDFLKHCQADH |
| | | LLDEKGRAQCLLQREMVQSLEQQLVLEKEKLSAMQAHLAGKM |
| | | ALTKASSVASSDKGSCCIVAAGSQGPVVPAWSGPREAPDSLFAV |
| | | RRHLWGSHGNSTFPEFLHNMDYFKFHNMRPPFTYATLIRWAILE |
| | | APEKQRTLNEIYHWFTRMFAFFRNHPATWKNAIRHNLSLHKCFV |
| | | RVESEKGAVWTVDELEFRKKRSQRPSRCSNPTPGP |
| 344 | FOXP3 | MPNPRPGKPSAPSLALGPSPGASPSWRAAPKASDLLGARGPGGT |
| | | FQGRDLRGGAHASSSSLNPMPPSQLQLPTLPLVMVAPSGARLGP |
| | | LPHLQALLQDRPHFMHQLSTVDAHARTPVLQVHPLESPAMISLT |
| | | PPTTATGVFSLKARPGLPPGINVASLEWVSREPALLCTFPNPSAPR |
| | | KDSTLSAVPQSSYPLLANGVCKWPGCEKVFEEPEDFLKHCQADH |
| | | LLDEKGRAQCLLQREMVQSLEQQLVLEKEKLSAMQAHLAGKM |
| | | ALTKASSVASSDKGSCCIVAAGSQGPVVPAWSGPREAPDSLFAV |
| | | RRHLWGSHGNSTFPEFLHNMDYFKFHNMRPPFTYATLIRWAILE |
| | | APEKQRTLNEIYHWFTRMFAFFRNHPATWKNAIRHNLSLHKCFV |
| | | RVESEKGAVWTVDELEFRKKR |
| 345 | FOXP3 | GGAHASSSSLNPMPPSQLQLPTLPLVMVAPSGARLGPLPHLQAL |
| | | LQDRPHFMHQLSTVDAHARTPVLQVHPLESPAMISLTPPTTATG |
| | | VFSLKARPGLPPGINVASLEWVSREPALLCTFPNPSAPRKDSTLS |
| | | AVPQSSYPLLANGVCKWPGCEKVFEEPEDFLKHCQADHLLDEK |
| | | GRAQCLLQREMVQSLEQQLVLEKEKLSAMQAHLAGKMALTKA |
| | | SSVASSDKGSCCIVAAGSQGPVVPAWSGPREAPDSLFAVRRHLW |
| | | GSHGNSTFPEFLHNMDYFKFHNMRPPFTYATLIRWAILEAPEKQ |
| | | RTLNEIYHWFTRMFAFFRNHPATWKNAIRHNLSLHKCFVRVESE |
| | | KGAVWTVDELEFRKKR |
| 346 | STAT5B | MAVWIQAQQLQGEALHQMQALYGQHFPIEVRHYLSQWIESQA |
| | | WDSVDLDNPQENIKATQLLEGLVQELQKKAEHQVGEDGFLLKI |
| | | KLGHYATQLQNTYDRCPMELVRCIRHILYNEQRLVREANNGSSP |
| | | AGSLADAMSQKHLQINQTFEELRLVTQDTENELKKLQQTQEYFII |
| | | QYQESLRIQAQFGPLAQLSPQERLSRETALQQKQVSLEAWLQRE |
| | | AQTLQQYRVELAEKHQKTLQLLRKQQTIILDDELIQWKRRQQLA |
| | | GNGGPPEGSLDVLQSWCEKLAEIIWQNRQQIRRAEHLCQQLPIPG |
| | | PVEEMLAEVNATITDIISALVTSTFIIEKQPPQVLKTQTKFAATVR |
| | | LLVGGKLNVHMNPPQVKATIISEQQAKSLLKNENTRNDYSGEIL |
| | | NNCCVMEYHQATGTLSAHFRNMSLKRIKRSDRRGAESVTEEKF |
| | | TILFESQFSVGGNELVFQVKTLSLPVVVIVHGSQDNNATATVLW |
| | | DNAFAEPGRVPFAVPDKVLWPQLCEALNMKFKAEVQSNRGLTK |
| | | ENLVFLAQKLFNNSSSHLEDYSGLSVSWSQFNRENLPGRNYTFW |
| | | QWFDGVMEVLKKHLKPHWNDGAILGFVNKQQAHDLLINKPDG |
| | | TFLLRFSDSEIGGITIAWKFDSQERMFWNLMPFTTRDFSIRSLADR |
| | | LGDLNYLIYVFPDRPKDEVYSKYYTPVPCESATAKAVDGYVKPQ |
| | | IKQVVPEFVNASADAGGGSATYMDQAPSPAVCPQAHYNMYPQ |
| | | NPDSVLDTDGDFDLEDTMDVARRVEELLGRPMDSQWIPHAQS |
| 347 | HELIOS | METEAIDGYITCDNELSPEREHSNMAIDLTSSTPNGQHASPSHMT |
| | | STNSVKLEMQSDEECDRKPLSREDEIRGHDEGSSLEEPLIESSEVA |
| | | DNRKVQELQGEGGIRLPNGKLKCDVCGMVCIGPNVLMVHKRSH |
| | | TGERPFHCNQCGASFTQKGNLLRHIKLHSGEKPFKCPFCSYACRR |
| | | RDALTGHLRTHSVGKPHKCNYCGRSYKQRSSLEEHKERCHNYL |
| | | QNVSMEAAGQVMSHHVPPMEDCKEQEPIMDNNISLVPFERPAVI |
| | | EKLTGNMGKRKSSTPQKFVGEKLMRFSYPDIHFDMNLTYEKEA |
| | | ELMQSHMMDQAINNAITYLGAEALHPLMQHPPSTIAEVAPVISS |
| | | AYSQVYHPNRIERPISRETADSHENNMDGPISLIRPKSRPQEREAS |
| | | PSNSCLDSTDSESSHDDHQSYQGHPALNPKRKQSPAYMKEDVK |
| | | ALDTTKAPKGSLKDIYKVFNGEGEQIRAFKCEHCRVLFLDHVMY |
| | | TIHMGCHGYRDPLECNICGYRSQDRYEFSSHIVRGEHTFH |

In some embodiments, a transcription factor encoded by an inventive polynucleotide has a sequence as listed in Table 28.

TABLE 29
Additional Accessory Sequences

| SEQ | | |
|---|---|---|
| ID NO | IRES | Sequence |
| 390 | CK 3′ UTR Ser | ccctgcagccgtcaccgtaagtttgaagttaccgcatatcagcctctgcttcccagcgcgtccaatt |
| | | cctgttcttattgtttcccctccaggcgttacgcgtgacgacgaactgtgtcgcagctaccacattatt |
| | | ccggagccttcattctcgcggctctgatcgt |
| 391 | CK 3′ UTR S2M | ggagaccgcggccacgccgagtaggatcgagggtacagtctcc |
| 392 | CK 3′ UTR | gacaccaggatcactcttgctctgacccgccctgtgtagaatagactcatgcttccctaagacctgg |
| | | atttcttcccaggcactttcacccgcctgccctgctccttcagtggactgcacccagggaggcggtc |
| | | tctgactgtcctttactttctattctggattgc |
| 393 | CK 5′ UTR 1 | AAACCCCCCTAAGCCGCCGCCGCCGCCACC |
| 394 | CK 5′ UTR 2 | CCCCCCCAACCCGTCACG |
| 395 | CK 5′ UTR 3 | GTCACG |
| 396 | SZ1 3′ UTR Scr | tctgcgcactcgtaatcagtactaacccccctttgtcggacactatgcgataatcgatccgcctttttc |
| | | accgccttcggaattttatttacctcaactgatcctggagtctctcttggttttcacggaggcctccgcc |
| | | ca |
| 397 | SZ1 S2M | ggagaccgcggccacgccgagtaggatcgagggtacagtctcc |
| 398 | SZ1 3′ UTR | ccccttgaaacccccgccccaggttcagtctctcttcatccctctgtcctgcatggtgatacaaagac |
| | | cctttgtggaccctaagccatgtagttgctgctccctccttccagttgtgaatattggtttctgttaatca |
| | | ca |
| 399 | SZ1 5′ UTR 1 | AAACCCCCCTAAGCCGCCGCCGCCGCCACC |
| 400 | SZ1 5′ UTR 2 | CCCCCCCAACCCGTCACG |
| 401 | SZ1 5′ UTR 3 | GTCACG |
| 402 | UTR1 | gTcacG |
| 403 | UTR2 | AATAAGAGAGAAAAGAAGAGTAAGAAGAAATATAAGAGC |
| | | CACC |
| 404 | UTR3 | cgaactagtattcttctggtccccacagactcagagagaacccgccacc |
| 405 | UTR4 | Agccacc |
| 406 | STOP1 | tgatAGctAaCtaG |
| 407 | STOP2 | tagtAGctAaCtaG |
| 408 | STOP3 | tGatGActGaGtGA |
| 409 | STOP4 | tagtagctagGtag |
| 410 | STOP5 | taa |
| 411 | STOP6 | taatagCtaaCtag |
| 412 | STOP7 | taaCtagCtaaCtag |

In some embodiments, a circular RNA or a precursor RNA (e.g., linear precursor RNA) disclosed herein comprises a sequence as listed in Table 29.

In some embodiments, a polynucleotide or a protein encoded by a polynucleotide contains a sequence with at least about 70%, 75%, 80%, 85%, 90%, 91%, 92%, 93%, 94%, 95%, 96%, 97%, 98%, 99%, or 99.5% similarity to one or more sequences disclosed herein. In some embodiments, a polynucleotide or a protein encoded by a polynucleotide contains a sequence that is identical to one or more sequences disclosed herein.

Preferred embodiments are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

Wesselhoeft et al. (2019) RNA Circularization Diminishes Immunogenicity and Can Extend Translation Duration In Vivo. Molecular Cell. 74(3), 508-520 and Wesselhoeft et al. (2018) Engineering circular RNA for Potent and Stable Translation in Eukaryotic Cells. Nature Communications. 9, 2629 are incorporated by reference in their entirety.

The invention is further described in detail by reference to the following examples but are not intended to be limited to the following examples. These examples encompass any and all variations of the illustrations with the intention of providing those of ordinary skill in the art with complete disclosure and description of how to make and use the subject invention and are not intended to limit the scope of what is regarded as the invention.

### Example 1

### Example 1A: External Homology Regions Allow for Circularization of Long Precursor RNA Using the Permuted Intron Exon (PIE) Circularization Strategy

A 1,100 nt sequence containing a full-length encephalomyocarditis virus (EMCV) IRES, a Gaussia luciferase (GLuc) expression sequence, and two short exon fragments of the permuted intron-exon (PIE) construct were inserted between the 3′ and 5′ introns of the permuted group I catalytic intron in the thymidylate synthase (Td) gene of the T4 phage. Precursor RNA was synthesized by run-off transcription. Circularization was attempted by heating the precursor RNA in the presence of magnesium ions and GTP, but splicing products were not obtained.

Perfectly complementary 9 nucleotide and 19 nucleotide long homology regions were designed and added at the 5′ and 3′ ends of the precursor RNA. Addition of these homology arms increased splicing efficiency from 0 to 16% for 9 nucleotide homology regions and to 48% for 19 nucleotide homology regions as assessed by disappearance of the precursor RNA band.

The splicing product was treated with RNase R. Sequencing across the putative splice junction of RNase R-treated splicing reactions revealed ligated exons, and digestion of the RNase R-treated splicing reaction with oligonucleotide-targeted RNase H produced a single band in contrast to two bands yielded by RNase H-digested linear precursor. This shows that circular RNA is a major product of the splicing reactions of precursor RNA containing the 9 or 19 nucleotide long external homology regions.

### Example 1B: Spacers that Conserve Secondary Structures of IRES and PIE Splice Sites Increase Circularization Efficiency

A series of spacers was designed and inserted between the 3′ PIE splice site and the IRES. These spacers were designed to either conserve or disrupt secondary structures within intron sequences in the IRES, 3′ PIE splice site, and/or 5′ splice site. The addition of spacer sequences designed to conserve secondary structures resulted in 87% splicing efficiency, while the addition of a disruptive spacer sequences resulted in no detectable splicing.

### Example 2

### Example 2A: Internal Homology Regions in Addition to External Homology Regions Creates a Splicing Bubble and Allows for Translation of Several Expression Sequences

Spacers were designed to be unstructured, non-homologous to the intron and IRES sequences, and to contain spacer-spacer homology regions. These were inserted between the 5′ exon and IRES and between the 3′ exon and expression sequence in constructs containing external homology regions, EMCV IRES, and expression sequences for Gaussia luciferase (total length: 1289 nt), Firefly luciferase (2384 nt), eGFP (1451 nt), human erythropoietin (1313 nt), and Cas9 endonuclease (4934 nt). Circularization of all 5 constructs was achieved. Circularization of constructs utilizing T4 phage and Anabaena introns were roughly equal. Circularization efficiency was higher for shorter sequences. To measure translation, each construct was transfected into HEK293 cells. Gaussia and Firefly luciferase transfected cells produced a robust response as measured by luminescence, human erythropoietin was detectable in the media of cells transfected with erythropoietin circRNA, and EGFP fluorescence was observed from cells transfected with EGFP circRNA. Co-transfection of Cas9 circRNA with sgRNA directed against GFP into cells constitutively expressing GFP resulted in ablated fluorescence in up to 97% of cells in comparison to an sgRNA-only control.

### Example 2B: Use of CVB3 IRES Increases Protein Production

Constructs with internal and external homology regions and differing IRES containing either Gaussia luciferase or Firefly luciferase expression sequences were made. Protein production was measured by luminescence in the supernatant of HEK293 cells 24 hours after transfection. The Coxsackievirus B3 (CVB3) IRES construct produced the most protein in both cases.

### Example 2C: Use of polyA or polyAC Spacers Increases Protein Production

Thirty nucleotide long polyA or polyAC spacers were added between the IRES and splice junction in a construct with each IRES that produced protein in example 2B. Gaussia luciferase activity was measured by luminescence in the supernatant of HEK293 cells 24 hours after transfection. Both spacers improved expression in every construct over control constructs without spacers.

### Example 3

HEK293 or HeLa Cells Transfected with Circular RNA Produce More Protein than Those Transfected with Comparable Unmodified or Modified Linear RNA.

HPLC-purified Gaussia luciferase-coding circRNA (CVB3-GLuc-pAC) was compared with a canonical unmodified 5′ methylguanosine-capped and 3′ polyA-tailed linear GLuc mRNA, and a commercially available nucleoside-modified (pseudouridine, 5-methylcytosine) linear GLuc mRNA (from Trilink). Luminescence was measured 24 h post-transfection, revealing that circRNA produced 811.2% more protein than the unmodified linear mRNA in HEK293 cells and 54.5% more protein than the modified mRNA. Similar results were obtained in HeLa cells and a comparison of optimized circRNA coding for human erythropoietin with linear mRNA modified with 5-methoxyuridine.

Luminescence data was collected over 6 days. In HEK293 cells, circRNA transfection resulted in a protein production half-life of 80 hours, in comparison with the 43 hours of unmodified linear mRNA and 45 hours of modified linear mRNA. In HeLa cells, circRNA transfection resulted in a protein production half-life of 116 hours, in comparison with the 44 hours of unmodified linear mRNA and 49 hours of modified linear mRNA. CircRNA produced substantially more protein than both the unmodified and modified linear mRNAs over its lifetime in both cell types.

### Example 4

### Example 4A: Purification of circRNA by RNase Digestion, HPLC Purification, and Phosphatase Treatment Decreases Immunogenicity. Completely Purified Circular RNA is Significantly Less Immunogenic than Unpurified or Partially Purified Circular RNA. Protein Expression Stability and Cell Viability are Dependent on Cell Type and Circular RNA Purity

Human embryonic kidney 293 (HEK293) and human lung carcinoma A549 cells were transfected with:
products of an unpurified GLuc circular RNA splicing reaction,
products of RNase R digestion of the splicing reaction,
products of RNase R digestion and HPLC purification of the splicing reaction, or
products of RNase digestion, HPLC purification, and phosphatase treatment of the splicing reaction.

RNase R digestion of splicing reactions was insufficient to prevent cytokine release in A549 cells in comparison to untransfected controls.

The addition of HPLC purification was also insufficient to prevent cytokine release, although there was a significant reduction in interleukin-6 (IL-6) and a significant increase in interferon-α1 (IFN-α1) compared to the unpurified splicing reaction.

The addition of a phosphatase treatment after HPLC purification and before RNase R digestion dramatically reduced the expression of all upregulated cytokines assessed in A549 cells. Secreted monocyte chemoattractant protein 1 (MCP1), IL-6, IFN-α1, tumor necrosis factor α (TNFα), and IFNγ inducible protein-10 (IP-10) fell to undetectable or untransfected baseline levels.

There was no substantial cytokine release in HEK293 cells. A549 cells had increased GLuc expression stability and cell viability when transfected with higher purity circular RNA. Completely purified circular RNA had a stability phenotype similar to that of transfected 293 cells.

### Example 4B: Circular RNA does not Cause Significant Immunogenicity and is not a RIG-I Ligand

A549 cells were transfected with the products of a splicing reaction:

A549 cells were transfected with:

unpurified circular RNA,

high molecular weight (linear and circular concatenations) RNA,

circular (nicked) RNA,

an early fraction of purified circular RNA (more overlap with nicked RNA peak),

a late fraction of purified circular RNA (less overlap with nicked RNA peak),

introns excised during circularization, or

vehicle (i.e. untransfected control).

Precursor RNA was separately synthesized and purified in the form of the splice site deletion mutant (DS) due to difficulties in obtaining suitably pure linear precursor RNA from the splicing reaction. Cytokine release and cell viability was measured in each case.

Robust IL-6, RANTES, and IP-10 release was observed in response to most of the species present within the splicing reaction, as well as precursor RNA. Early circRNA fractions elicited cytokine responses comparable to other non-circRNA fractions, indicating that even relatively small quantities of linear RNA contaminants are able to induce a substantial cellular immune response in A549 cells. Late circRNA fractions elicited no cytokine response in excess of that from untransfected controls. A549 cell viability 36 hours post-transfection was significantly greater for late circRNA fractions compared with all of the other fractions.

RIG-I and IFN-β1 transcript induction upon transfection of A549 cells with late circRNA HPLC fractions, precursor RNA or unpurified splicing reactions were analyzed. Induction of both RIG-I and IFN-β1 transcripts were weaker for late circRNA fractions than precursor RNA and unpurified splicing reactions. RNase R treatment of splicing reactions alone was not sufficient to ablate this effect. Addition of very small quantities of the RIG-I ligand 3p-hpRNA to circular RNA induced substantial RIG-I transcription. In HeLa cells, transfection of RNase R-digested splicing reactions induced RIG-I and IFN-β1, but purified circRNA did not. Overall, HeLa cells were less sensitive to contaminating RNA species than A549 cells.

A time course experiment monitoring RIG-I, IFN-β1, IL-6, and RANTES transcript induction within the first 8 hours after transfection of A549 cells with splicing reactions or fully purified circRNA did not reveal a transient response to circRNA. Purified circRNA similarly failed to induce pro-inflammatory transcripts in RAW264.7 murine macrophages.

A549 cells were transfected with purified circRNA containing an EMCV IRES and EGFP expression sequence. This failed to produce substantial induction of pro-inflammatory transcripts. These data demonstrate that non-circular components of the splicing reaction are responsible for the immunogenicity observed in previous studies and that circRNA is not a natural ligand for RIG-I.

### Example 5

### Circular RNA Avoids Detection by TLRs.

TLR 3, 7, and 8 reporter cell lines were transfected with multiple linear or circular RNA constructs and secreted embryonic alkaline phosphatase (SEAP) was measured.

Linearized RNA was constructed by deleting the intron and homology arm sequences. The linear RNA constructs were then treated with phosphatase (in the case of capped RNAs, after capping) and purified by HPLC.

None of the attempted transfections produced a response in TLR7 reporter cells. TLR3 and TLR8 reporter cells were activated by capped linearized RNA, polyadenylated linearized RNA, the nicked circRNA HPLC fraction, and the early circRNA fraction. The late circRNA fraction and m1ψ-mRNA did not provoke TLR-mediated response in any cell line.

In a second experiment, circRNA was linearized using two methods: treatment of circRNA with heat in the presence of magnesium ions and DNA oligonucleotide-guided RNase H digestion. Both methods yielded a majority of full-length linear RNA with small amounts of intact circRNA. TLR3, 7, and 8 reporter cells were transfected with circular RNA, circular RNA degraded by heat, or circular RNA degraded by RNase H, and SEAP secretion was measured 36 hours after transfection. TLR8 reporter cells secreted SEAP in response to both forms of degraded circular RNA, but did not produce a greater response to circular RNA transfection than mock transfection. No activation was observed in TLR3 and TLR7 reporter cells for degraded or intact conditions, despite the activation of TLR3 by in vitro transcribed linearized RNA.

### Example 6

Unmodified Circular RNA Produces Increased Sustained In Vivo Protein Expression than Linear RNA.

Mice were injected and HEK293 cells were transfected with unmodified and m1ψ-modified human erythropoietin (hEpo) linear mRNAs and circRNAs. Equimolar transfection of m1ψ-mRNA and unmodified circRNA resulted in robust protein expression in HEK293 cells. hEpo linear mRNA and circRNA displayed similar relative protein expression patterns and cell viabilities in comparison to GLuc linear mRNA and circRNA upon equal weight transfection of HEK293 and A549 cells.

In mice, hEpo was detected in serum after the injection of hEpo circRNA or linear mRNA into visceral adipose. hEpo detected after the injection of unmodified circRNA decayed more slowly than that from unmodified or m1ψ-mRNA and was still present 42 hours post-injection. Serum hEpo rapidly declined upon the injection of unpurified circRNA splicing reactions or unmodified linear mRNA. Injection of unpurified splicing reactions produced a cytokine response detectable in serum that was not observed for the other RNAs, including purified circRNA.

### Example 7

### Circular RNA can be Effectively Delivered In Vivo or In Vitro Via Lipid Nanoparticles.

Purified circular RNA was formulated into lipid nanoparticles (LNPs) with the ionizable lipidoid cKK-E12 (Dong et al., 2014; Kauffman et al., 2015). The particles formed uniform multilamellar structures with an average size, polydispersity index, and encapsulation efficiency similar to that of particles containing commercially available control linear mRNA modified with 5moU.

Purified hEpo circRNA displayed greater expression than 5moU-mRNA when encapsulated in LNPs and added to HEK293 cells. Expression stability from LNP-RNA in HEK293 cells was similar to that of RNA delivered by transfection reagent, with the exception of a slight delay in decay for both 5moU-mRNA and circRNA. Both unmodified circRNA and 5moU-mRNA failed to activate RIG-I/IFN-β1 in vitro.

In mice, LNP-RNA was delivered by local injection into visceral adipose tissue or intravenous delivery to the liver. Serum hEpo expression from circRNA was lower but comparable with that from 5moU-mRNA 6 hours after delivery in both cases. Serum hEpo detected after adipose injection of unmodified LNP-circRNA decayed more slowly than that from LNP-5moU-mRNA, with a delay in expression decay present in serum that was similar to that noted in vitro, but serum hEpo after intravenous injection of LNP-circRNA or LNP-5moU-mRNA decayed at approximately the same rate. There was no increase in serum cytokines or local RIG-I, TNFα, or IL-6 transcript induction in any of these cases.

### Example 8

### Example 8A: Expression and Functional Stability by IRES in HEK293, HepG2, and 1C1C7 Cells

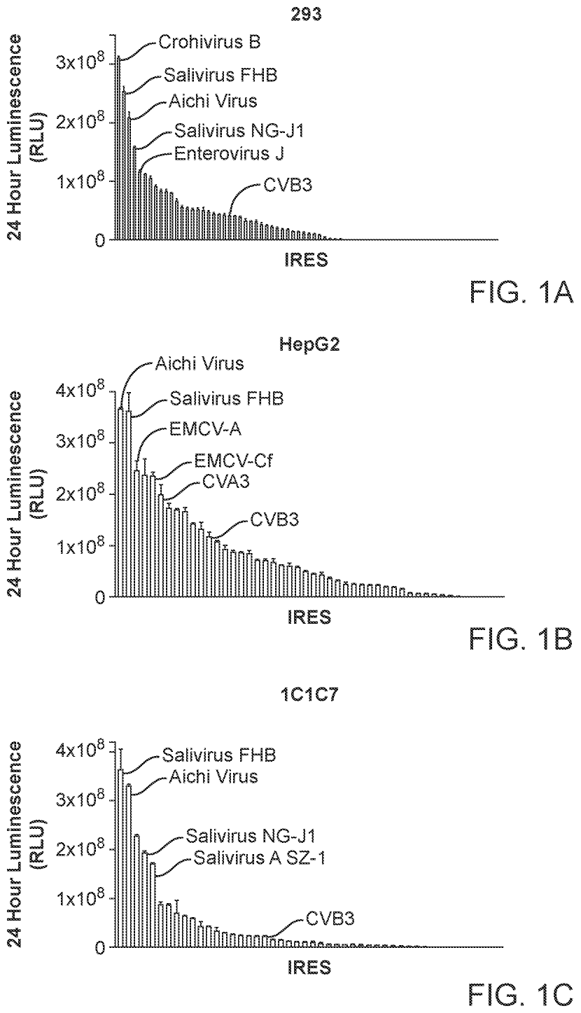

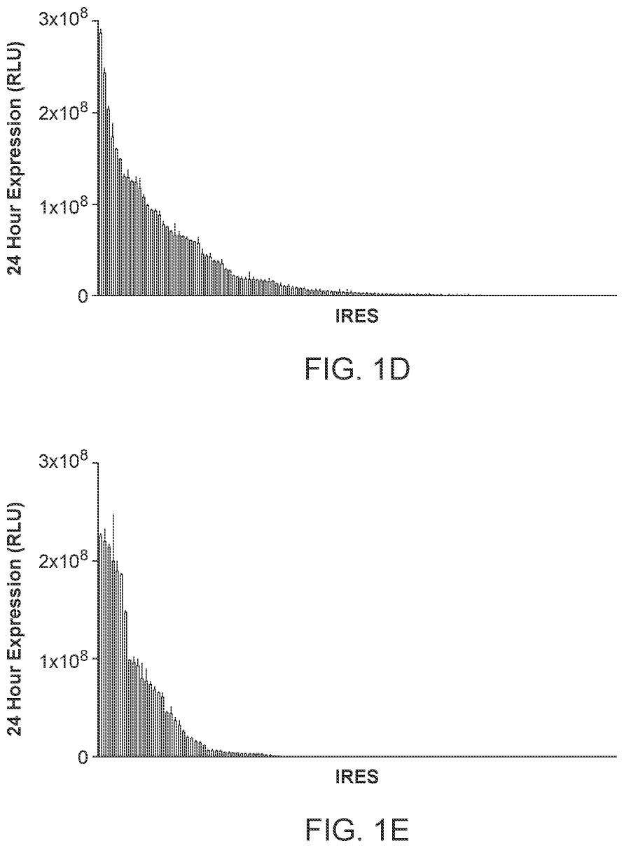

Constructs including Anabaena intron/exon regions, a Gaussia luciferase expression sequence, and varying IRES were circularized. 100 ng of each circularization reaction was separately transfected into 20,000 HEK293 cells, HepG2 cells, and 1C1C7 cells using Lipofectamine MessengerMax. Luminescence in each supernatant was assessed after 24 hours as a measure of protein expression. In HEK293 cells, constructs including Crohivirus B, Salivirus FHB, Aichi Virus, Salivirus HG-J1, and Enterovirus J IRES produced the most luminescence at 24 hours (FIG. 1A). In HepG2 cells, constructs including Aichi Virus, Salivirus FHB, EMCV-Cf, and CVA3 IRES produced high luminescence at 24 hours (FIG. 1B). In 1C1C7 cells, constructs including Salivirus FHB, Aichi Virus, Salivirus NG-J1, and Salivirus A SZ-1 IRES produced high luminescence at 24 hours (FIG. 1C).

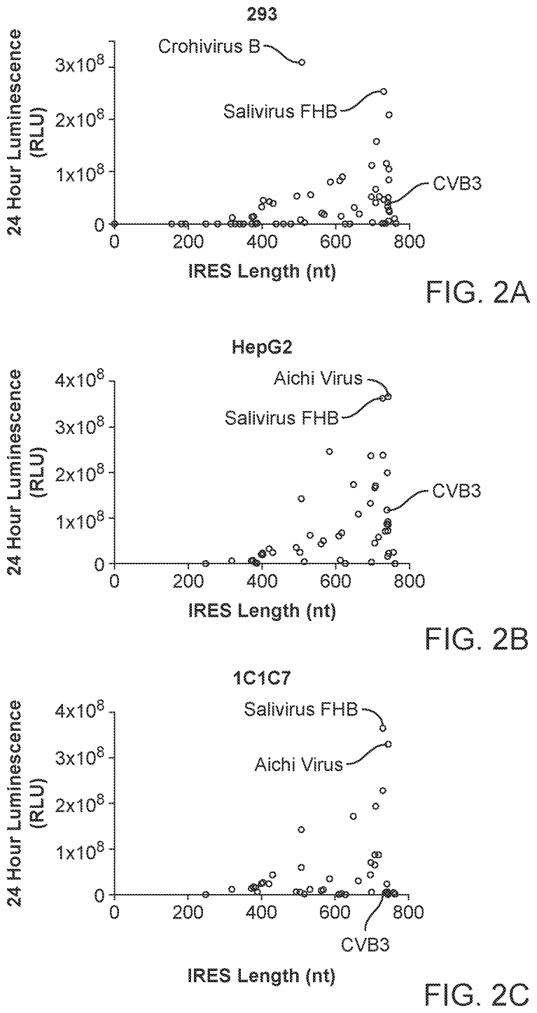

A trend of larger IRES producing greater luminescence at 24 hours was observed. Shorter total sequence length tends to increase circularization efficiency, so selecting a high expression and relatively short IRES may result in an improved construct. In HEK293 cells, a construct using the Crohivirus B IRES produced the highest luminescence, especially in comparison to other IRES of similar length (FIG. 2A). Expression from IRES constructs in HepG2 and 1C1C7 cells plotted against IRES size are in FIGS. 2B and 2C.

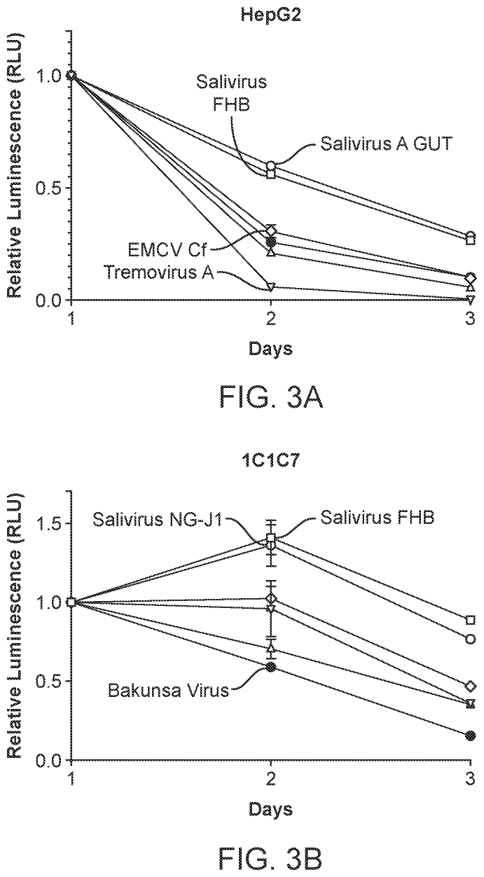

Functional stability of select IRES constructs in HepG2 and 1C1C7 cells were measured over 3 days. Luminescence from secreted Gaussia luciferase in supernatant was measured every 24 hours after transfection of 20,000 cells with 100 ng of each circularization reaction, followed by complete media replacement. Salivirus A GUT and Salivirus FHB exhibited the highest functional stability in HepG2 cells, and Salivirus N-J1 and Salivirus FHB produced the most stable expression in 1C1C7 cells (FIGS. 3A and 3B).

### Example 8B: Screening of Additional IRES

Functional stability of additional IRES constructs in HEK293 cells were measured. Briefly, 5′ untranslated regions (UTRs) of interest were identified from GenBank. Selected UTRs were truncated to 675 nt from the 5′ end and inserted into a circular RNA backbone construct encoding Gaussia Luciferase (Gluc) and in front of the Gluc coding region. The circular RNAs were transfected into HEK293 cells. After 24 hours, the supernatants were collected and the luminescence from secreted Gluc protein was measured using commercially available reagents. The results are depicted in FIGS. 1D and 1E and Table 30, suggesting that many natural IRES sequences enhance the protein expression in a circular RNA context.

TABLE 30

| SEQ ID NO | IRES | Expression |
|---|---|---|
| 413 | RhPV | 1.10E+05 |
| 414 | Halastavi arva (1x mut) | 9.46E+04 |
| 415 | Oscivirus | 4.55E+07 |
| 416 | Cadicivirus B | 2.10E+05 |
| 417 | PSIV (2x mut for Xba1) | 9.70E+04 |
| 418 | PSIV IGR | 1.01E+05 |
| 419 | PV Mahoney | 1.09E+05 |
| 420 | REV A | 9.44E+04 |
| 421 | Tropivirus A | 9.52E+04 |
| 422 | Symapivirus A | 1.27E+05 |
| 423 | Sakobuvirus A FFUP1 (1x mut) | 8.82E+06 |
| 424 | Rosavirus C NFSM6F | 6.84E+05 |
| 425 | Rosavirus 2 GA7403 | 5.05E+06 |
| 426 | Rhimavirus A | 8.42E+05 |
| 427 | Rafivirus LPXYC222841 | 2.22E+05 |
| 428 | Rafivirus WHWGGF74766 | 4.53E+06 |
| 429 | Poecivirus BCCH-449 | 3.43E+05 |
| 430 | Megirivirus A LY | 1.80E+06 |
| 431 | Megirivirus E | 1.10E+07 |
| 432 | Megirivirus C | 1.24E+05 |
| 433 | Ludopivirus | 1.05E+05 |
| 434 | Livupivirus | 2.10E+05 |
| 435 | Aichivirus A FSS693 | 6.25E+07 |
| 436 | Aichivirus KVGH | 1.72E+07 |
| 437 | Aichivirus DV | 7.79E+07 |
| 438 | Murine Kobuvirus 1 | 1.60E+07 |
| 439 | Porcine Kobuvirus K-30 | N/A |
| 440 | Porcine Kobuvirus XX | 1.32E+07 |
| 441 | Caprine Kobuvirus 12Q108 | 2.87E+08 |
| 442 | Rabbit Kobuvirus | 3.73E+07 |
| 443 | Aalivirus | 2.65E+05 |
| 444 | Grusopivirus A | 1.09E+05 |
| 445 | Grusopivirus B | 2.12E+05 |
| 446 | Yancheng osbecks grenadier anchovy | 1.57E+06 |
| | picornavirus | |
| 447 | Turkey Gallivirus M176 | 4.37E+05 |
| 448 | Falcovirus A1 | 1.48E+05 |
| 449 | Tremovirus B | 1.31E+05 |
| 450 | Didelphis aurita HAV | 1.38E+05 |
| 451 | Hepatovirus G1 | 1.41E+05 |
| 452 | Hepatovirus D | 1.47E+06 |
| 453 | Hepatovirus H2 | 1.08E+05 |
| 454 | Hepatovirus I | 8.79E+05 |
| 455 | Hepatovirus C | 5.08E+05 |
| 456 | Fipivirus A | 2.69E+05 |
| 457 | Fipivirus C | 1.09E+05 |
| 458 | Fipivirus E | 1.10E+05 |
| 459 | Aquamavirus | 4.51E+06 |
| 460 | Avisivirus A | 1.91E+05 |
| 461 | Avisivirus B | 8.68E+04 |
| 462 | Crohivirus A | 9.96E+04 |
| 463 | Kunsagivirus B | 8.01E+04 |
| 464 | Limnipivirus A | 8.30E+04 |
| 465 | Limnipivirus C | 1.35E+05 |
| 466 | Orivirus | 6.09E+05 |
| 467 | HAV FH1 | 1.24E+05 |
| 468 | HAV HM175 | 4.96E+05 |
| 469 | Parechovirus F | 6.56E+05 |
| 470 | Parechovirus D | 3.10E+05 |
| 471 | Parechovirus C | 1.24E+06 |
| 472 | Ljungan Virus 87-012 | 2.00E+06 |
| 473 | Parechovirus A2 | 1.80E+07 |
| 474 | Parechovirus A3 | 3.58E+06 |
| 475 | Parechovirus A8 | 1.61E+07 |
| 476 | Parechovirus A17 | 1.20E+06 |
| 477 | Potamipivirus A | 8.43E+05 |
| 478 | Potamipivirus B | 7.20E+05 |
| 479 | Beihai Conger Picornavirus | 1.15E+06 |
| 480 | Porcine Sapelovirus JD2011 | N/A |
| 481 | Porcine Sapelovirus A2 | 4.34E+06 |
| 482 | Simian Sapelovirus 1 | 6.55E+07 |
| 483 | Simian Sapelovirus 2 | 4.24E+07 |
| 484 | Rabovirus C | 2.49E+06 |
| 485 | Rabovirus A NYC-B10 | 1.24E+06 |
| 486 | Parabovirus C | 1.83E+07 |
| 487 | Parabovirus B | 7.85E+06 |
| 488 | Parabovirus A3 | 2.44E+08 |
| 489 | Felipivirus 127F | 8.92E+06 |
| 490 | Boosepivirus A | 7.07E+07 |
| 491 | Boosepivirus B | 1.17E+08 |
| 492 | Phacovirus Pf-CHK1 | 5.87E+06 |
| 493 | HRVC3 QPM | 1.64E+07 |
| 494 | HRVB27 | 2.04E+08 |
| 495 | HRVA73 | 1.08E+08 |
| 496 | EV L | 6.49E+07 |
| 497 | EV K | 7.52E+07 |
| 498 | EV J 1631 | 9.88E+07 |
| 499 | EV J N125 | 2.90E+07 |
| 500 | EV I | 1.31E+08 |
| 501 | EV F1 BEV 261 | 1.12E+07 |
| 502 | EV D94 | 9.25E+07 |
| 503 | PV3 | 1.25E+08 |
| 504 | EV C102 | 8.85E+07 |
| 505 | EV 30 | 5.48E+06 |
| 506 | SA5 | 1.61E+08 |
| 507 | EV A114 | 1.50E+08 |
| 508 | Mobovirus A | 3.44E+06 |
| 509 | Burpengary Virus | 1.09E+07 |
| 510 | Hunnivirus A1 | 1.61E+06 |
| 511 | Hunnivirus A2 | 6.38E+06 |
| 512 | Ia Io | 1.35E+06 |
| 513 | Taura Syndrome Virus | 8.30E+05 |
| 514 | ABPV | 6.48E+05 |
| 515 | BRAV-2 | 3.98E+06 |
| 516 | BRBV-1 | 3.34E+06 |
| 517 | ERAV-1 U188 | N/A |
| 518 | GFTV | 1.23E+06 |
| 519 | SAFV V13C | 9.32E+07 |
| 520 | SAV P-113 | 4.37E+07 |
| 521 | VHEV | 1.74E+08 |
| 522 | TRV NGS910 | 3.84E+07 |
| 523 | EMCV2 RD1338 | 1.97E+06 |
| 524 | EMCV1 JZ1203 | N/A |
| 525 | EMCV1 AnrB-3741 | 2.55E+06 |
| 526 | Cosavirus D1 | 2.11E+06 |
| 527 | Cosavirus B1 | 1.91E+06 |
| 528 | Cosavirus A SH1 | 2.16E+06 |
| 529 | Malagasivirus B | 5.05E+06 |
| 530 | Mosavirus A2 SZAL6 | 8.27E+06 |
| 531 | SVV | 1.06E+06 |
| 532 | PTV A | 7.29E+05 |
| 533 | PTV B | 6.02E+06 |
| 534 | Tottorivirus | 2.76E+07 |
| 535 | Posavirus 1 | 1.55E+06 |
| 536 | A105-675 | 2.18E+07 |
| 537 | A110-675 | 1.24E+08 |
| 538 | 18-675 | 6.04E+07 |
| 539 | A115-675 | 5.93E+07 |
| 540 | A73-675 | 1.30E+08 |
| 541 | Kobuvirus 16317 | 2.03E+07 |
| 542 | Aichivirus Chshc7 | 1.87E+07 |
| 543 | Aichivirus Goiania | 1.66E+07 |
| 544 | Aichivirus ETHP4 | 1.78E+07 |
| 545 | Aichivirus DVI2169 | 2.98E+06 |
| 546 | Aichivirus DVI2321 | 6.63E+07 |
| 547 | Aichivirus rat08 | 3.51E+07 |
| 548 | Aichivirus Rt386 | 5.71E+07 |
| 549 | Norway Rat Pestivirus | N/A |
| 550 | Porcine Kobuvirus GS2 | 44200000 |
| 551 | Kobuvirus SZAL6 | 98850000 |
| 552 | Kobuvirus sheep TB3 | N/A |
| 553 | Pronghorn antelope pestivirus | 1.35E+06 |
| 554 | Porcine pestivirus isolate Bungowannah | 1.10E+07 |
| 555 | Porcine pestivirus 1 | 9.46E+04 |
| 556 | Pestivirus giraffe-1 | 4.72E+05 |
| 557 | Classical swine fever virus | 3.16E+05 |
| 558 | Human pegivirus isolate JD2B1I | 6.85E+05 |
| 559 | Human pegivirus isolate GBV-C-ZJ | N/A |
| 560 | Human pegivirus isolate JD2B8C | 5.36E+05 |
| 561 | Hepatitis GB virus A | N/A |
| 562 | Simian pegivirus | 8.56E+04 |
| 563 | Pegivirus I | 8.02E+04 |
| 564 | Pegivirus K | 8.07E+04 |
| 565 | Theiler's disease-associated virus | 7.84E+04 |
| 566 | Rodent pegivirus | 1.79E+05 |
| 567 | Human pegivirus 2 | 3.14E+05 |
| 568 | GB virus C/Hepatitis G virus | 1.36E+05 |
| 569 | Equine Pegivirus 1 | 8.80E+04 |
| 570 | Culex theileri flavivirus | 8.52E+04 |
| 571 | Bussuquara virus | 8.20E+04 |
| 572 | Zika Virus | 8.61E+04 |
| 573 | Yokose virus | 8.55E+04 |
| 574 | Wesselsbron virus | N/A |
| 575 | Equine hepacivirus | 8.40E+04 |
| 576 | Hepacivirus B | 8.84E+04 |
| 577 | Hepacivirus I | 7.50E+04 |
| 578 | Hepacivirus J | 7.65E+04 |
| 579 | Hepacivirus K | 8.91E+04 |
| 580 | Icavirus | 4.41E+06 |
| 581 | Antarctic penguin virus A | 8.42E+04 |
| 582 | Forest pouched giant rat arterivirus | N/A |
| 583 | Avisivirus Pf-CHK1 | 1.19E+05 |
| 584 | Avian paramyxovirus penguin | 9.91E+04 |
| 585 | Newcastle disease virus | 8.86E+04 |
| 586 | Bat Hp-betacoronavirus | 8.47E+04 |
| 587 | Basella alba endornavirus | 7.65E+04 |
| 588 | Ball python nidovirus | 8.25E+04 |
| 589 | Bat sapelovirus | 8.05E+04 |
| 590 | Bat Picornavirus 3 | N/A |
| 591 | Bat Picornavirus 2 | 7.99E+07 |
| 592 | Bat Picornavirus 1 | 1.85E+07 |
| 593 | Bat Iflavirus | 9.76E+04 |
| 594 | Bat dicibavirus | 7.43E+04 |
| 595 | Betacoronavirus HKU24 | 8.96E+04 |
| 596 | Betacoronavirus England 1 | 8.74E+04 |
| 597 | Boone cardiovirus 1 | 2.62E+06 |
| 598 | Breda virus | 1.16E+05 |
| 599 | Bovine viral diarrhea virus 3 | 2.70E+06 |
| 600 | Bovine rhinitis A virus | 3.62E+06 |
| 601 | Bovine picornavirus isolate TCH6 | 1.21E+05 |
| 602 | Bovine nidovirus TCH5 | 1.17E+05 |
| 603 | Bovine hepacivirus | 1.89E+05 |
| 604 | Botrytis cinerea mitovirus 4 RdRp | 9.68E+04 |
| 605 | Botrytis cinerea mitovirus 2 RdRp | 8.73E+04 |
| 606 | Canine picodicistrovirus strain 209 | 2.79E+06 |
| 607 | Canine distemper virus | 3.02E+05 |
| 608 | Canine kobuvirus | 1.48E+08 |
| 609 | Camel alphacoronavirus | 2.48E+05 |
| 610 | Cripavirus | 1.95E+05 |
| 611 | Human coxsackievirus A2 | 7.75E+07 |
| 612 | Coronavirus AcCoV-JC34 | 1.82E+05 |
| 613 | Chicken picornavirus 3 | 9.13E+04 |
| 614 | Chicken picornavirus 1 | 1.21E+05 |
| 615 | Chicken orivirus 1 | 3.16E+05 |
| 616 | Chicken gallivirus 1 | 1.51E+07 |
| 617 | Chicken calicivirus | 1.28E+05 |
| 618 | Carp picornavirus 1 | 1.13E+05 |
| 619 | Falcon picornavirus | 3.08E+06 |
| 620 | Equine rhinitis B virus 1 | 1.01E+05 |
| 621 | Equine rhinitis A virus | 3.73E+05 |
| 622 | Equine arteritis virus | 1.89E+05 |
| 623 | Enterovirus sp. isolate CPML | 6.83E+07 |
| 624 | Enterovirus AN12 | 3.87E+06 |
| 625 | Dolphin morbillivirus | 1.22E+05 |
| 626 | Dianke virus | 1.35E+05 |
| 627 | Guereza hepacivirus | 1.38E+05 |
| 628 | Grapevine associated narnavirus-1 | 1.30E+05 |
| 629 | Goat torovirus | 1.19E+05 |
| 630 | Foot-and-mouth disease virus O isolate | 1.12E+05 |
| 631 | Feline infectious peritonitis virus | 1.35E+05 |
| 632 | Farmington virus | 1.22E+05 |
| 633 | Avian infectious bronchitis virus | 2.84E+05 |
| 634 | Human rhinovirus 1 | 7.40E+07 |
| 635 | EV22 | 1.95E+07 |
| 636 | Human TMEV-like cardiovirus | 4.48E+07 |
| 637 | Human coronavirus 229E | N/A |
| 638 | Hubei zhaovirus-like virus 1 | 1.03E+05 |
| 639 | Hubei tombus-like virus 9 | 9.28E+04 |
| 640 | Hubei tombus-like virus 32 | 9.23E+04 |
| 641 | Hubei sobemo-like virus 3 | 1.17E+05 |
| 642 | Hubei picorna-like virus 2 | 1.95E+05 |
| 643 | Hepacivirus P | 6.04E+05 |
| 644 | Harrier picornavirus 1 | 1.47E+05 |
| 645 | Kunsagivirus 1 | 4.15E+05 |
| 646 | Kagoshima-2-24-KoV | 9.30E+07 |
| 647 | Kashmir bee virus | 1.65E+05 |
| 648 | Jingmen picorna-like virus | 9.32E+04 |
| 649 | Mumps virus | 1.47E+05 |
| 650 | Mouse Mosavirus | 9.00E+04 |
| 651 | Miniopterus schreibersii picornavirus 1 | 6.05E+06 |
| 652 | Linda virus | 7.37E+05 |
| 653 | Lesavirus 2 | 3.67E+07 |
| 654 | Lesavirus 1 | 6.37E+06 |
| 655 | Phopivirus strain NewEngland | 1.06E+05 |
| 656 | Pestivirus strain Aydin | 3.11E+06 |
| 657 | Quail picornavirus QPV1 | 6.55E+07 |
| 658 | Porcine sapelovirus 1 | N/A |
| 659 | Porcine reproductive and respiratory | 1.29E+05 |
| | syndrome virus 2 | |
| 660 | Porcine enterovirus 9 | 3.20E+07 |
| 661 | Pigeon picornavirus B | 1.24E+05 |
| 662 | Picornavirus HK21 | 4.09E+05 |
| 663 | Picornavirales Tottori-HG1 | 9.54E+04 |
| 664 | Rodent hepatovirus | 1.39E+05 |
| 665 | Rinderpest virus | 4.26E+05 |
| 666 | Rabovirus A | 2.88E+06 |
| 667 | Shingleback nidovirus 1 | 2.62E+05 |
| 668 | Seneca valley virus | 1.46E+07 |
| 669 | Sclerotinia sclerotiorum dsRNA mycovirus-L | 1.69E+05 |
| 670 | Yak enterovirus | 6.19E+06 |
| 671 | Wobbly possum disease virus | 2.60E+05 |
| 672 | Avian orthoreovirus segment S1 | 4.37E+05 |
| 673 | Caprine Kobuvirus d10 | 2.20E+08 |
| 674 | Caprine Kobuvirus d20 | 2.00E+08 |
| 675 | Caprine Kobuvirus d30 | 1.87E+08 |
| 676 | Caprine Kobuvirus d40 | 2.15E+08 |
| 677 | Caprine Kobuvirus d50 | 9.65E+07 |
| 678 | Picornavirales sp. isolate RtMruf-PicoV | 2.26E+08 |
| 679 | Apodemus agrarius picornavirus strain | 1.90E+08 |
| | Longquan-Aa118 | |
| 680 | Niviventer confucianus picornavirus | 6.10E+07 |
| 681 | Bat picornavirus isolate BtRs-PicoV | 1.13E+06 |
| 682 | Rhinolophus picornavirus strain Guizhou- | N/A |
| | Rr100 | |
| 683 | Rhinolophus picornavirus strain Henan- | 3.85E+05 |
| | Rf265 | |
| 684 | Human enterovirus C105 | 5.49E+05 |
| 685 | Human poliovirus 1 strain NIE1116623 | 3.94E+05 |
| 686 | Human enterovirus 109 | 4.92E+05 |
| 687 | Human poliovirus 2 strain NIE0811460 | 2.59E+07 |
| 688 | Bovine picornavirus | 3.82E+06 |
| 689 | Human poliovirus 1 strain EQG1419328 | 2.44E+05 |
| 690 | Human poliovirus 2 isolate IS_061 | 5.84E+06 |
| 691 | Coxsackievirus B5 | N/A |
| 692 | Coxsackievirus A10 | N/A |

### Example 9

### Expression and Functional Stability by IRES in Jurkat Cells.

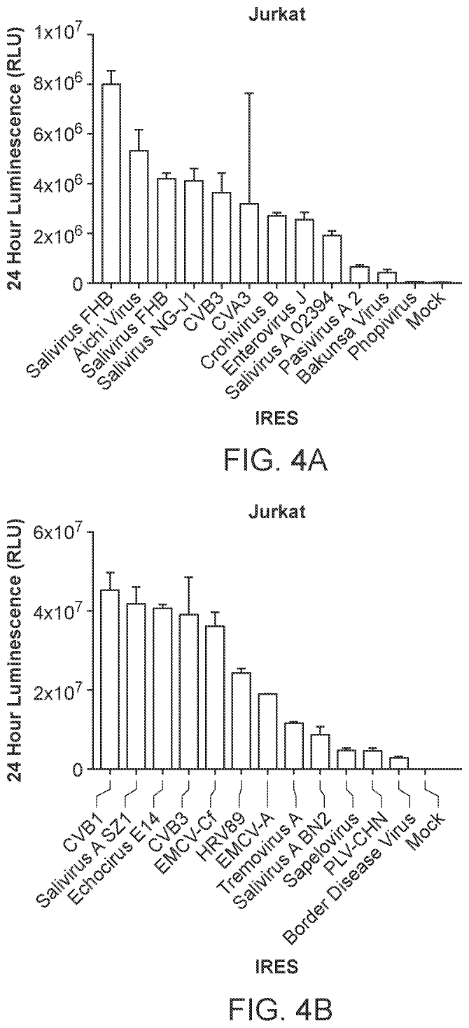

2 sets of constructs including Anabaena intron/exon regions, a Gaussia luciferase expression sequence, and a subset of previously tested IRES were circularized. 60,000 Jurkat cells were electroporated with 1 μg of each circularization reaction. Luminescence from secreted Gaussia luciferase in supernatant was measured 24 hours after electroporation. A CVB3 IRES construct was included in both sets for comparison between sets and to previously defined IRES efficacy. CVB1 and Salivirus A SZ1 IRES constructs produced the most expression at 2 4h. Data can be found in FIGS. 4A and 4B.

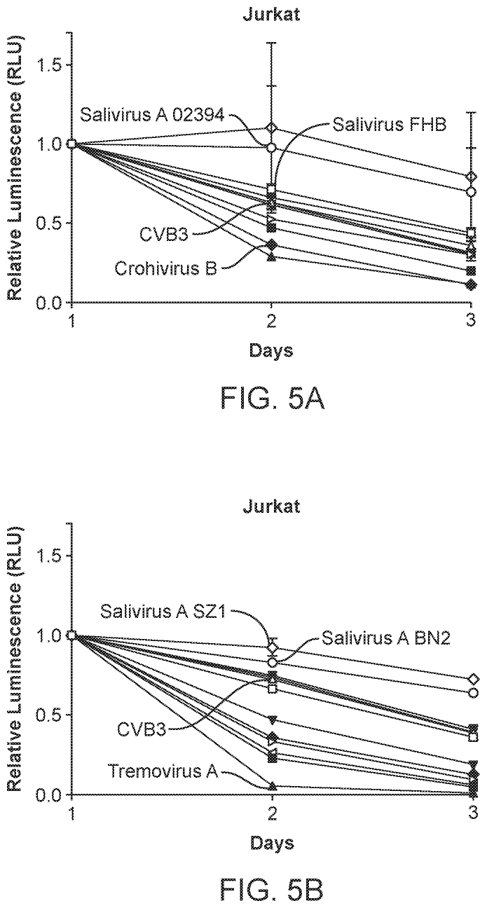

Functional stability of the IRES constructs in each round of electroporated Jurkat cells was measured over 3 days. Luminescence from secreted Gaussia luciferase in supernatant was measured every 24 hours after electroporation of 60,000 cells with 1 μg of each circularization reaction, followed by complete media replacement (FIGS. 5A and 5B).

Salivirus A SZ1 and Salivirus A BN2 IRES constructs had high functional stability compared to other constructs.

### Example 10

### Expression, Functional Stability, and Cytokine Release of Circular and Linear RNA in Jurkat Cells.

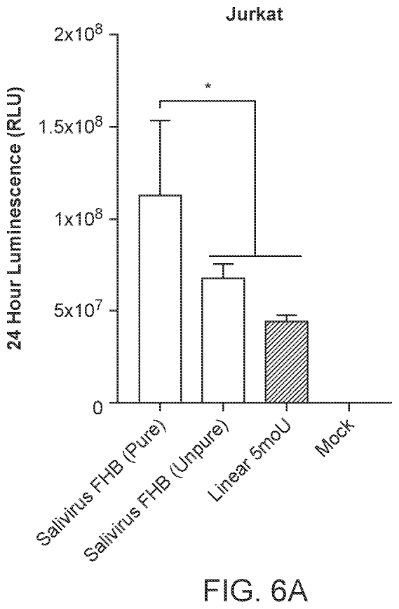

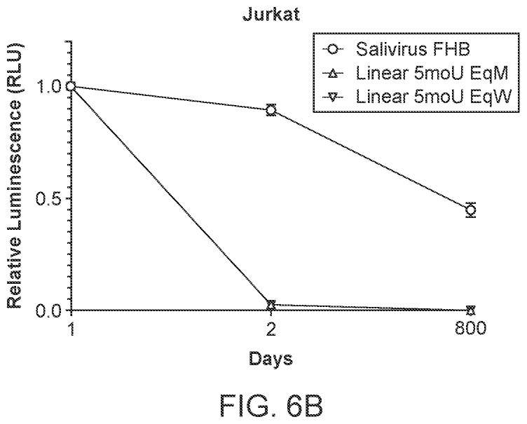

A construct including Anabaena intron/exon regions, a Gaussia luciferase expression sequence, and a Salivirus FHB IRES was circularized. mRNA including a Gaussia luciferase expression sequence and a ˜150 nt polyA tail, and modified to replace 100% of uridine with 5-methoxy uridine (5moU) is commercially available and was purchased from Trilink. 5moU nucleotide modifications have been shown to improve mRNA stability and expression (Bioconjug Chem. 2016 Mar. 16; 27(3):849-53). Expression of modified mRNA, circularization reactions (unpure), and circRNA purified by size exclusion HPLC (pure) in Jurkat cells were measured and compared (FIG. 6A). Luminescence from secreted Gaussia luciferase in supernatant was measured 24 hours after electroporation of 60,000 cells with 1 μg of each RNA species.

Luminescence from secreted Gaussia luciferase in supernatant was measured every 24 hours after electroporation of 60,000 cells with 1 ug of each RNA species, followed by complete media replacement. A comparison of functional stability data of modified mRNA and circRNA in Jurkat cells over 3 days is in FIG. 6B.

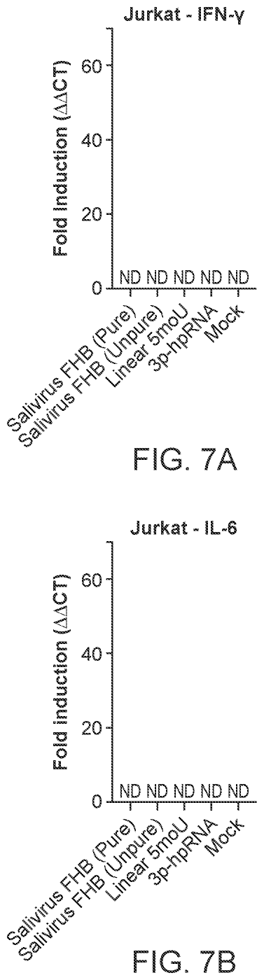

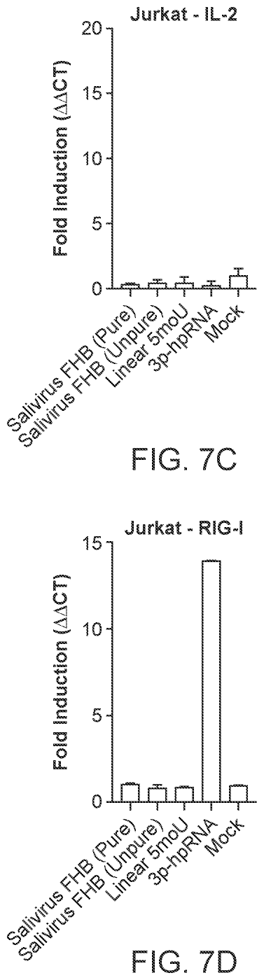

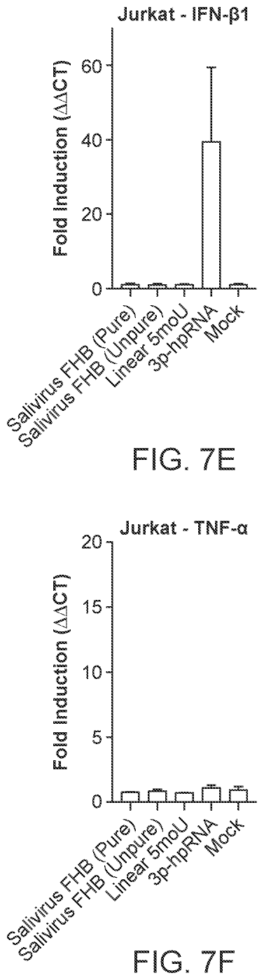

IFNγ (FIG. 7A), IL-6 (FIG. 7B), IL-2 (FIG. 7C), RIG-I (FIG. 7D), IFN-β1 (FIG. 7E), and TNFα (FIG. 7F) transcript induction was measured 18 hours after electroporation of 60,000 Jurkat cells with 1 μg of each RNA species described above and 3p-hpRNA (5′ triphosphate hairpin RNA, which is a known RIG-I agonist).

### Example 11

### Expression of Circular and Linear RNA in Monocytes and Macrophages.

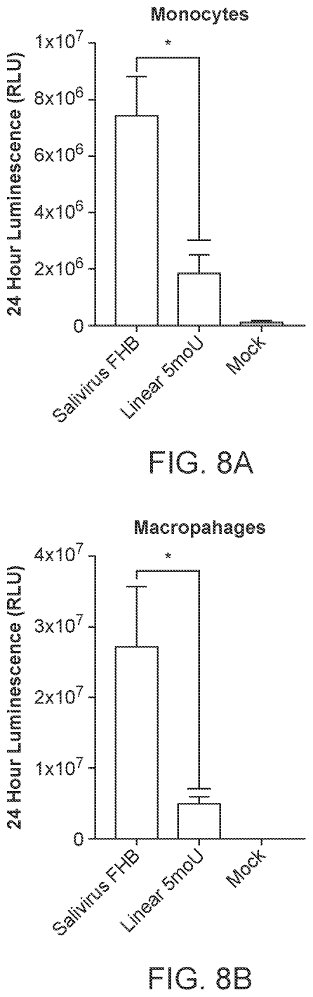

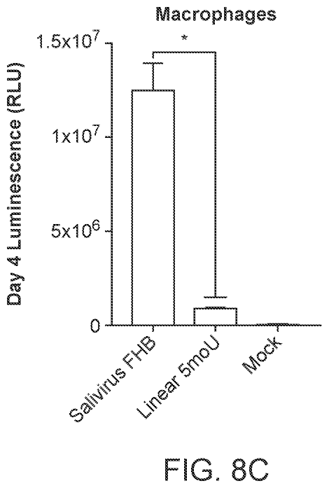

A construct including Anabaena intron/exon regions, a Gaussia luciferase expression sequence, and a Salivirus FHB IRES was circularized. mRNA including a Gaussia luciferase expression sequence and a ˜150 nt polyA tail, and modified to replace 100% of uridine with 5-methoxy uridine (5moU) was purchased from Trilink. Expression of circular and modified mRNA was measured in human primary monocytes (FIG. 8A) and human primary macrophages (FIG. 8B). Luminescence from secreted Gaussia luciferase in supernatant was measured 24 hours after electroporation of 60,000 cells with 1 μg of each RNA species. Luminescence was also measured 4 days after electroporation of human primary macrophages with media changes every 24 hours (FIG. 8C). The results can be found in FIG. 8. The difference in luminescence was statistically significant in each case (p<0.05).

### Example 12

### Expression and Functional Stability by IRES in Primary T Cells.

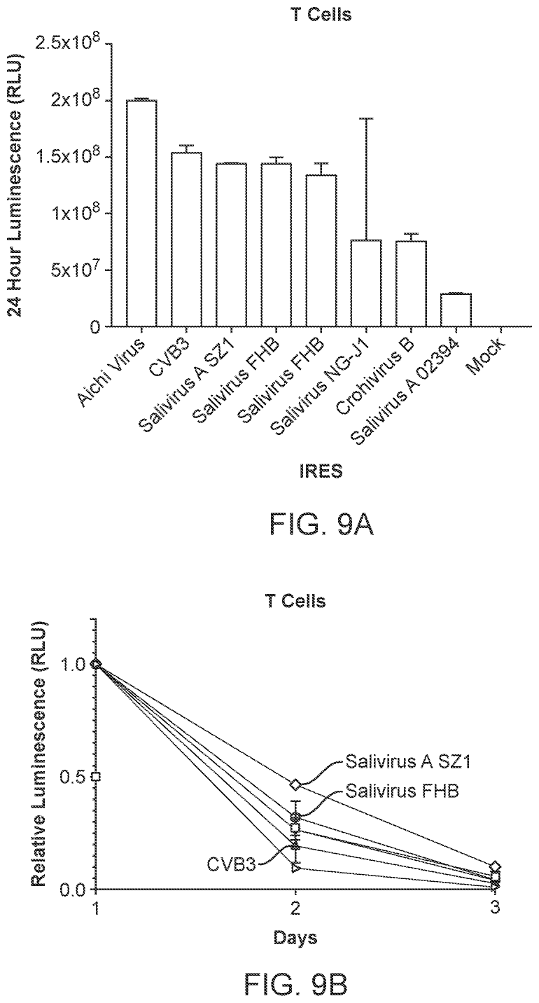

Constructs including Anabaena intron/exon regions, a Gaussia luciferase expression sequence, and a subset of previously tested IRES were circularized and reaction products were purified by size exclusion HPLC. 150,000 primary human CD3+ T cells were electroporated with 1 μg of each circRNA. Luminescence from secreted Gaussia luciferase in supernatant was measured 24 hours after electroporation (FIG. 9A). Aichi Virus and CVB3 IRES constructs had the most expression at 24 hours.

Luminescence was also measured every 24 hours after electroporation for 3 days in order to compare functional stability of each construct (FIG. 9B). The construct with a Salivirus A SZ1 IRES was the most stable.

### Example 13

### Expression and Functional Stability of Circular and Linear RNA in Primary T Cells and PBMCs.

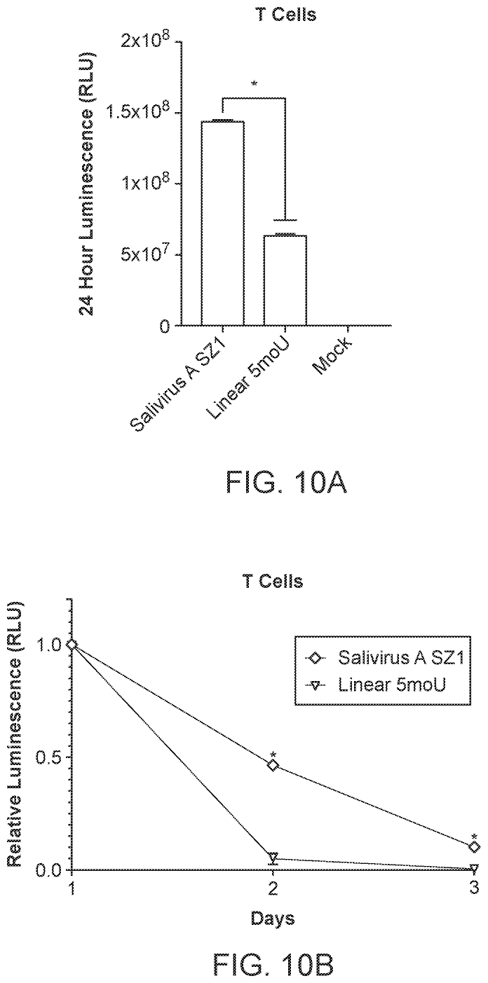

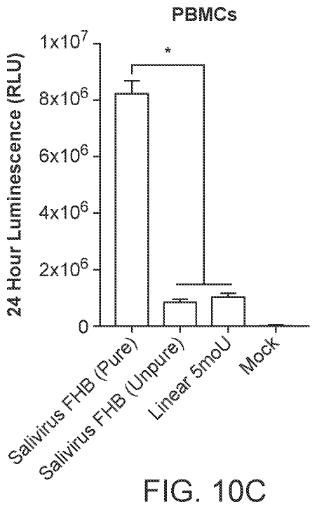

Constructs including Anabaena intron/exon regions, a Gaussia luciferase expression sequence, and a Salivirus A SZ1 IRES or Salivirus FHB IRES were circularized. mRNA including a Gaussia luciferase expression sequence and a ˜150 nt polyA tail, and modified to replace 100% of uridine with 5-methoxy uridine (5moU) and was purchased from Trilink. Expression of Salivirus A SZ1 IRES HPLC purified circular and modified mRNA was measured in human primary CD3+ T cells. Expression of Salivirus FHB HPLC purified circular, unpurified circular and modified mRNA was measured in human PBMCs. Luminescence from secreted Gaussia luciferase in supernatant was measured 24 hours after electroporation of 150,000 cells with 1 μg of each RNA species. Data for primary human T cells is shown in FIGS. 10A and 10B, and data for PBMCs is shown in FIG. 10C. The difference in expression between the purified circular RNA and unpurified circular RNA or linear RNA was significant in each case (p<0.05).

Luminescence from secreted Gaussia luciferase in primary T cell supernatant was measured every 24 hours after electroporation over 3 days in order to compare construct functional stability. Data is shown in FIG. 10B. The difference in relative luminescence from the day 1 measurement between purified circular RNA and linear RNA was significant at both day 2 and day 3 for primary T cells.

### Example 14

### Circularization Efficiency by Permutation Site in Anabaena Intron.

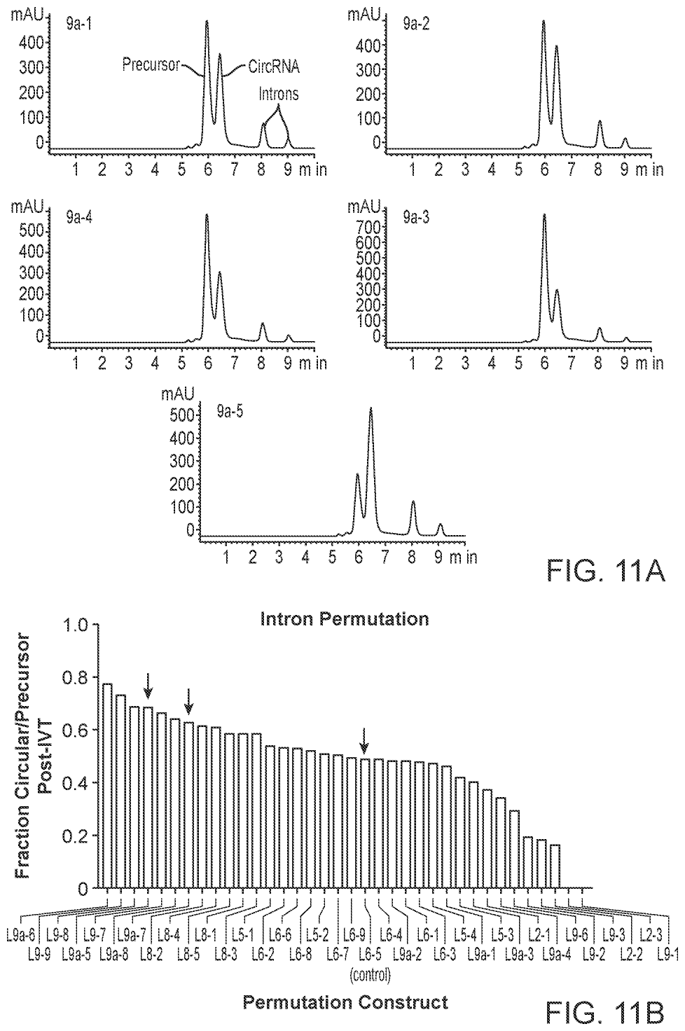

RNA constructs including a CVB3 IRES, a Gaussia luciferase expression sequence, Anabaena intron/exon regions, spacers, internal homology regions, and homology arms were produced. Circularization efficiency of constructs using the traditional Anabaena intron permutation site and 5 consecutive permutations sites in P9 was measured by HPLC. HPLC chromatograms for the 5 consecutive permutation sites in P9 are shown in FIG. 11A.

Circularization efficiency was measured at a variety of permutation sites. Circularization efficiency is defined as the area under the HPLC chromatogram curve for each of: circRNA/(circRNA+ precursor RNA). Ranked quantification of circularization efficiency at each permutation site is in FIG. 11B. 3 permutation sites (indicated in FIG. 11B) were selected for further investigation.

Circular RNA in this example was circularized by in vitro transcription (IVT) then purified via spin column. Circularization efficiency for all constructs would likely be higher if the additional step of incubation with Mg2+ and guanosine nucleotide were included; however, removing this step allowed for comparison between, and optimization of, circular RNA constructs. This level of optimization is especially useful for maintaining high circularization efficiency with large RNA constructs, such as those encoding chimeric antigen receptors.

### Example 15

### Circularization Efficiency of Alternative Introns.

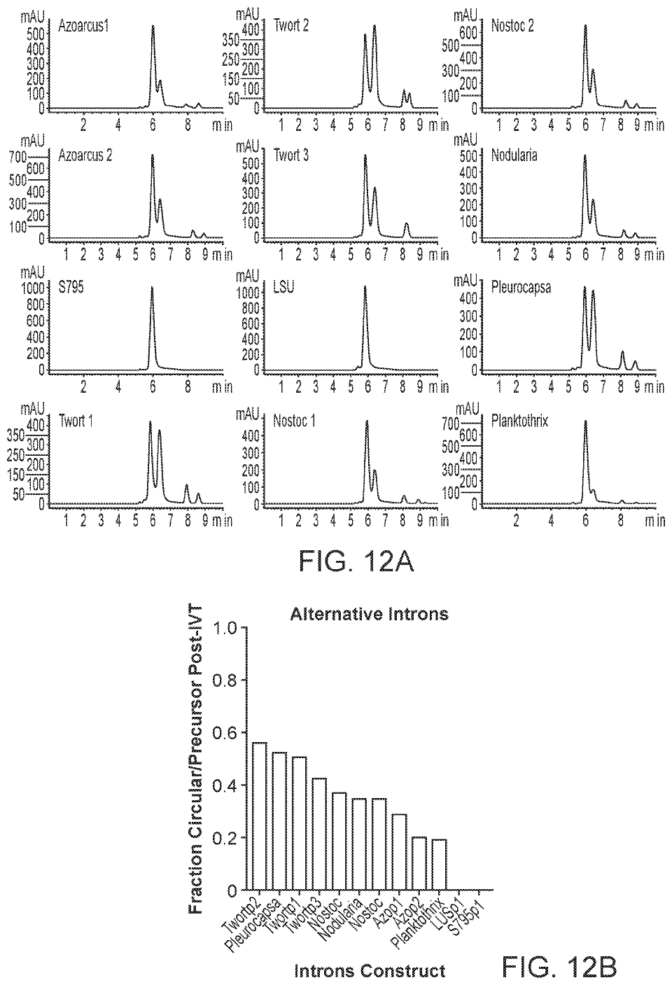

Precursor RNA containing a permuted group 1 intron of variable species origin or permutation site and several constant elements including: a CVB3 IRES, a Gaussia luciferase expression sequence, spacers, internal homology regions, and homology arms were created. Circularization data can be found in FIG. 12. FIG. 12A shows chromatograms resolving precursor, CircRNA and introns. FIG. 12B provides ranked quantification of circularization efficiency, based on the chromatograms shown in FIG. 12A, as a function of intron construct.

Circular RNA in this example was circularized by in vitro transcription (IVT) then spin column purification. Circularization efficiency for all constructs would likely be higher if the additional step of incubation with Mg2+ and guanosine nucleotide were included; however, removing this step allows for comparison between, and optimization of, circular RNA constructs. This level of optimization is especially useful for maintaining high circularization efficiency with large RNA constructs, such as those encoding chimeric antigen receptors.

### Example 16

### Circularization Efficiency by Homology Arm Presence or Length.

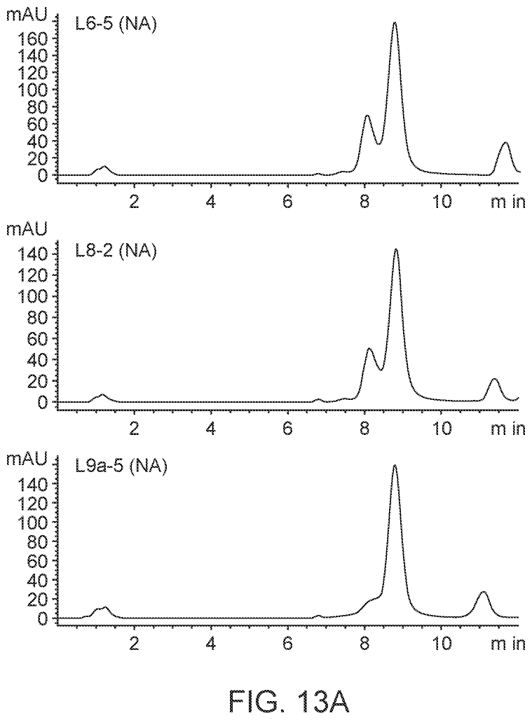

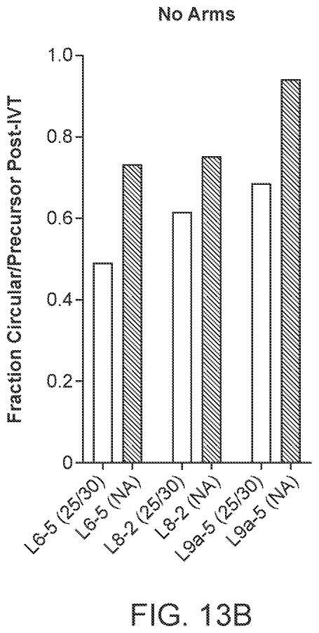

RNA constructs including a CVB3 IRES, a Gaussia luciferase expression sequence, Anabaena intron/exon regions, spacers, and internal homology regions were produced. Constructs representing 3 Anabaena intron permutation sites were tested with 30 nt, 25% GC homology arms or without homology arms (“NA”). These constructs were allowed to circularize without an Mg²⁺ incubation step. Circularization efficiency was measured and compared. Data can be found in FIGS. 13A and 13B. Circularization efficiency was higher for each construct lacking homology arms. FIG. 13A provides ranked quantification of circularization efficiency; FIG. 13B provides chromatograms resolving precursor, circRNA and introns.

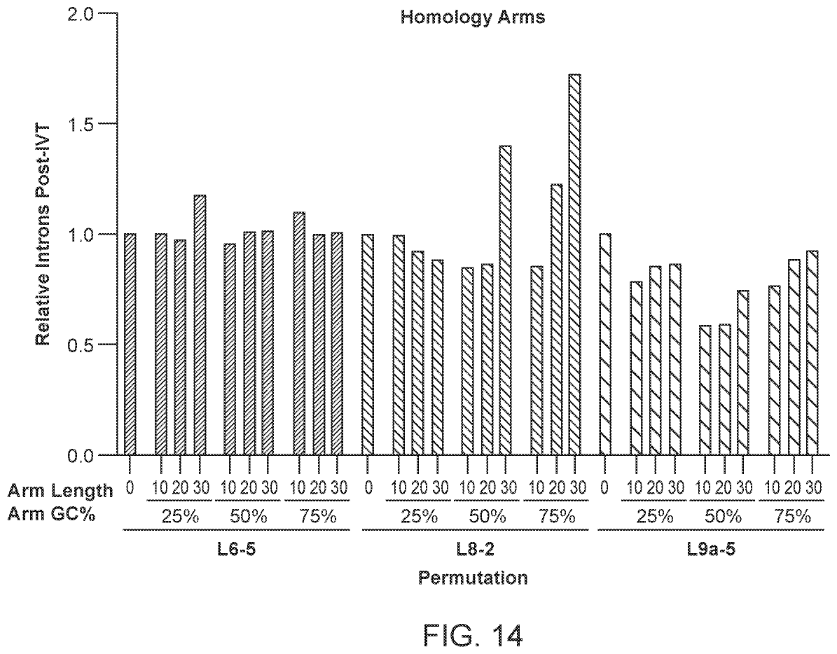

For each of the 3 permutation sites, constructs were created with 10 nt, 20 nt, and 30 nt arm lengths and 25%, 50%, and 75% GC content. Splicing efficiency of these constructs was measured and compared to constructs without homology arms (FIG. 14). Splicing efficiency is defined as the proportion of free introns relative to the total RNA in the splicing reaction.

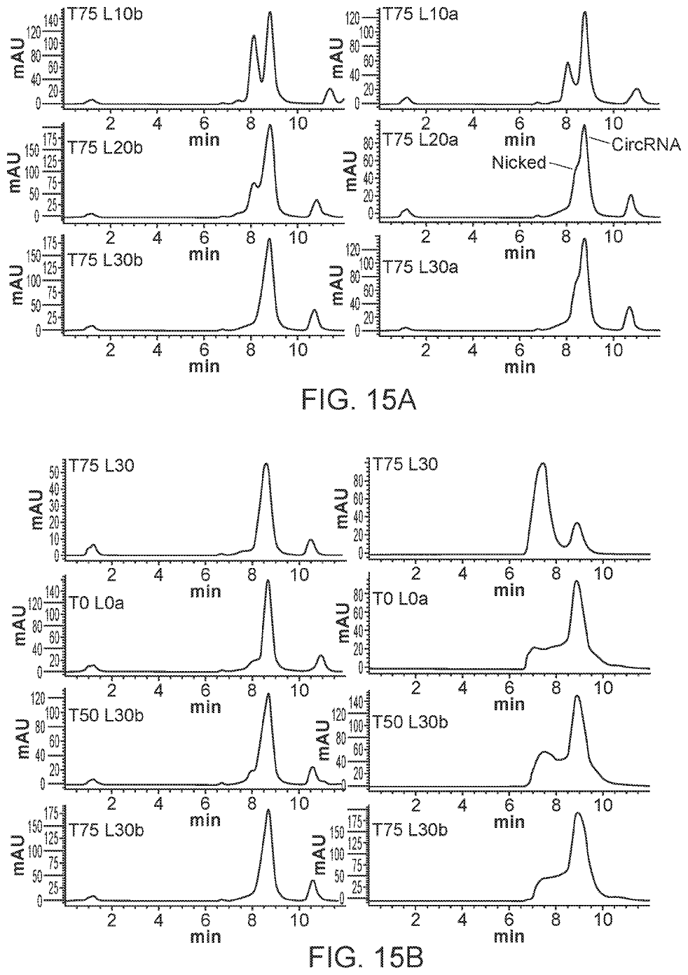

FIG. 15A (left) shows HPLC chromatograms indicating the contribution of strong homology arms to improved splicing efficiency. Top left: 75% GC content, 10 nt homology arms. Center left: 75% GC content, 20 nt homology arms. Bottom left: 75% GC content, 30 nt homology arms.

FIG. 15A (right) shows HPLC chromatograms showing increased splicing efficiency paired with increased nicking, appearing as a shoulder on the circRNA peak. Top right: 75% GC content, 10 nt homology arms. Center right: 75% GC content, 20 nt homology arms. Bottom right: 75% GC content, 30 nt homology arms.

FIG. 15 B (left) shows select combinations of permutation sites and homology arms hypothesized to demonstrate improved circularization efficiency.

FIG. 15 B (right) shows select combinations of permutation sites and homology arms hypothesized to demonstrate improved circularization efficiency, treated with E. coli polyA polymerase.

Circular RNA in this example was circularized by in vitro transcription (IVT) then spin-column purified. Circularization efficiency for all constructs would likely be higher if an additional Mg2+ incubation step with guanosine nucleotide were included; however, removing this step allowed for comparison between, and optimization of, circular RNA constructs. This level of optimization is especially useful for maintaining high circularization efficiency with large RNA constructs, such as those encoding chimeric antigen receptors.

### Example 17

### Circular RNA Encoding Chimeric Antigen Receptors

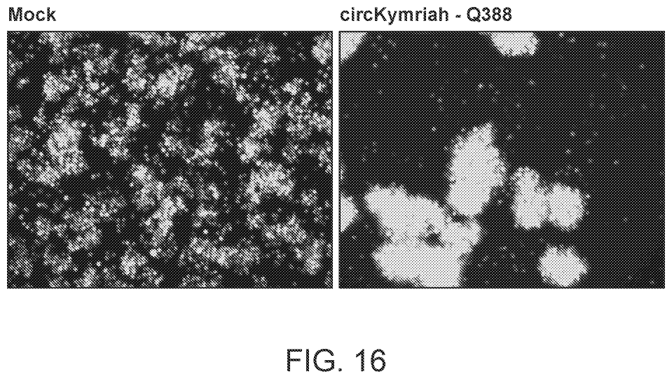

Constructs including Anabaena intron/exon regions, a Kymriah chimeric antigen receptors (CAR) expression sequence, and a CVB3 IRES were circularized. 100,000 human primary CD3+ T cells were electroporated with 500 ng of circRNA and co-cultured for 24 hours with Raji cells stably expressing GFP and firefly luciferase. Effector to target ratio (E:T ratio) 0.75:1. 100,000 human primary CD3+ T cells were mock electroporated and co-cultured as a control (FIG. 16).

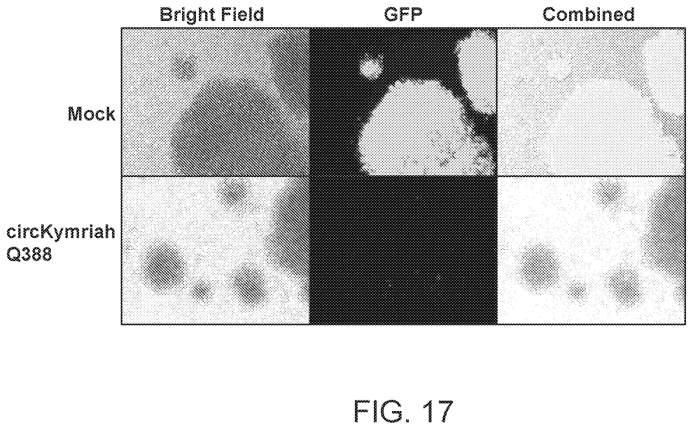

Sets of 100,000 human primary CD3+ T cells were mock electroporated or electroporated with 1 μg of circRNA then co-cultured for 48 hours with Raji cells stably expressing GFP and firefly luciferase E:T ratio 10:1 (FIG. 17).

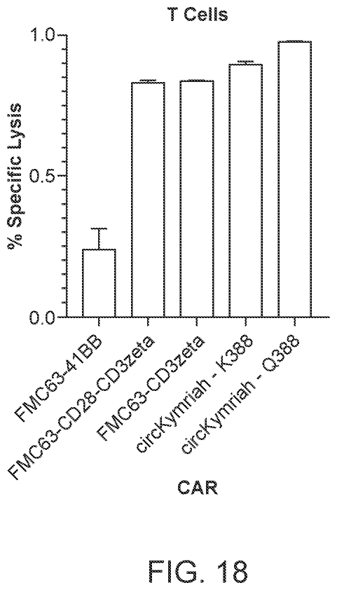

Quantification of specific lysis of Raji target cells was determined by detection of firefly luminescence (FIG. 18). 100,000 human primary CD3+ T cells either mock electroporated or electroporated with circRNA encoding different CAR sequences were co-cultured for 48 hours with Raji cells stably expressing GFP and firefly luciferase. % Specific lysis defined as 1-[CAR condition luminescence]/[mock condition luminescence]. E:T ratio 10:1.

### Example 18

### Expression and Functional Stability of Circular and Linear RNA in Jurkat Cells and Resting Human T Cells.

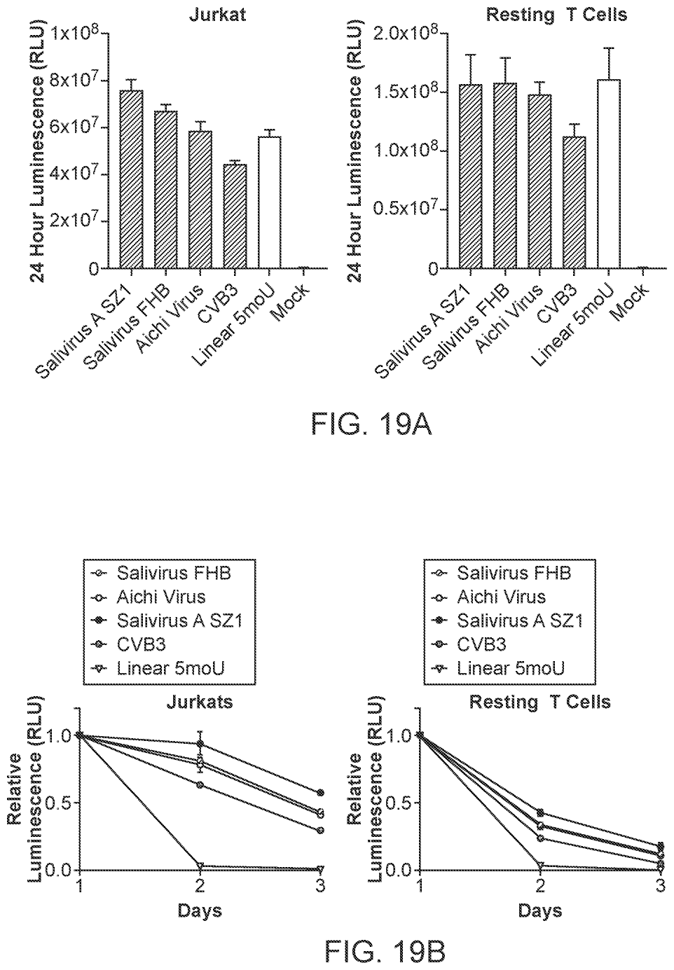

Constructs including Anabaena intron/exon regions, a Gaussia luciferase expression sequence, and a subset of previously tested IRES were circularized and reaction products were purified by size exclusion HPLC. 150,000 Jurkat cells were electroporated with 1 μg of circular RNA or 5moU-mRNA. Luminescence from secreted Gaussia luciferase in supernatant was measured 24 hours after electroporation (FIG. 19A left). 150,000 resting primary human CD3+ T cells (10 days post-stimulation) were electroporated with 1 μg of circular RNA or 5moU-mRNA. Luminescence from secreted Gaussia luciferase in supernatant was measured 24 hours after electroporation (FIG. 19A right).

Luminescence from secreted Gaussia luciferase in supernatant was measured every 24 hours after electroporation, followed by complete media replacement. Functional stability data shown in FIG. 19B. Circular RNA had more functional stability than linear RNA in each case, with a more pronounced difference in Jurkat cells.

### Example 19

IFN-β1, RIG-I, IL-2, IL-6, IFNγ, and TNFα Transcript Induction of Cells Electroporated with Linear RNA or Varying Circular RNA Constructs.

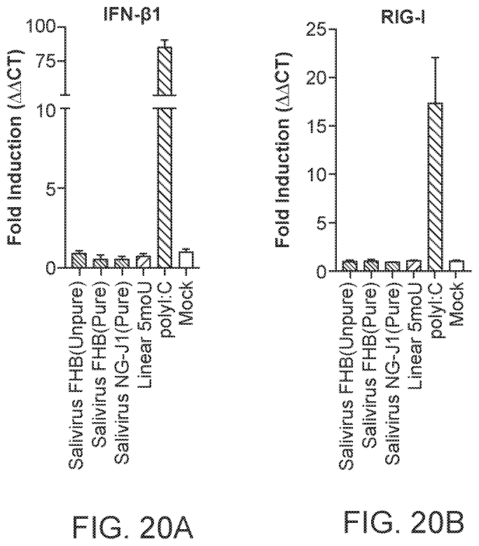

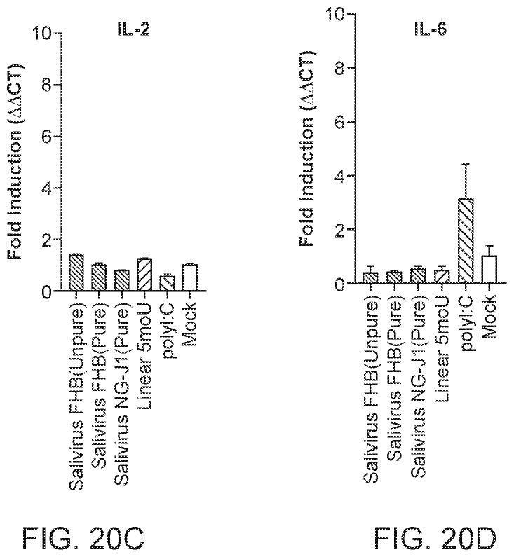

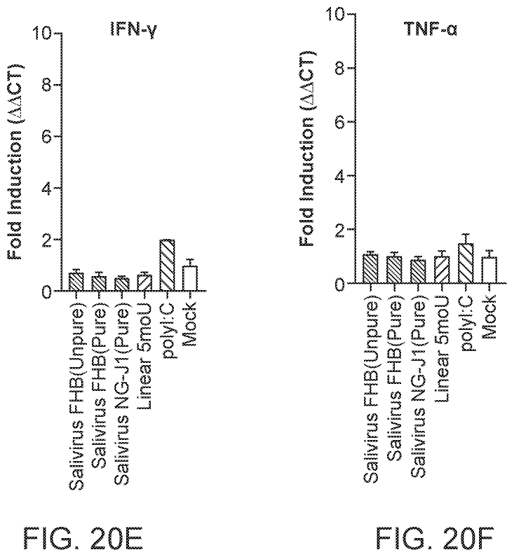

Constructs including Anabaena intron/exon regions, a Gaussia luciferase expression sequence, and a subset of previously tested IRES were circularized and reaction products were purified by size exclusion HPLC. 150,000 CD3+ human T cells were electroporated with 1 μg of circular RNA, 5moU-mRNA, or immunostimulatory positive control poly inosine:cytosine. IFN-β1 (FIG. 20A), RIG-I (FIG. 20B), IL-2 (FIG. 20C), IL-6 (FIG. 20D), IFNγ (FIG. 20E), and TNFα (FIG. 20F) transcript induction was measured 18 hours after electroporation.

### Example 20

Specific Lysis of Target Cells and IFNγ Transcript Induction by CAR Expressing Cells Electroporated with Different Amounts of Circular or Linear RNA; Specific Lysis of Target and Non-Target Cells by CAR Expressing Cells at Different E: T Ratios.

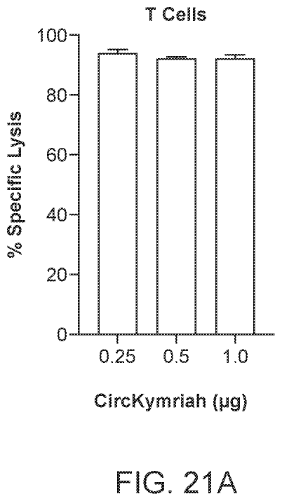

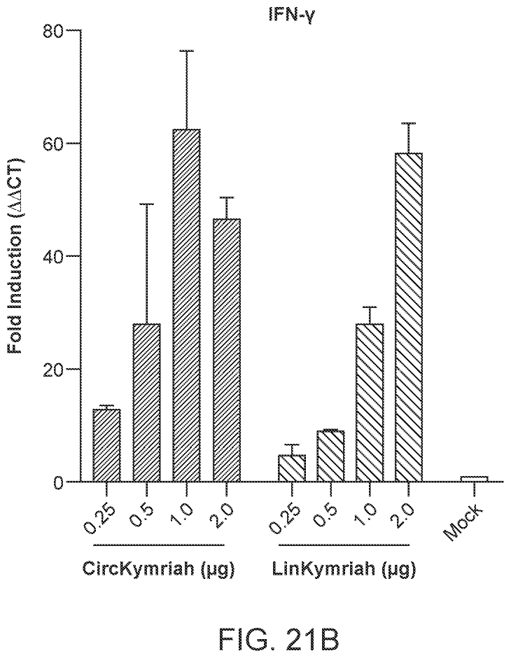

Constructs including Anabaena intron/exon regions, an anti-CD19 CAR expression sequence, and a CVB3 IRES were circularized and reaction products were purified by size exclusion HPLC. 150,000 human primary CD3+ T cells either mock electroporated or electroporated with different quantities of circRNA encoding an anti-CD19 CAR sequence were co-cultured for 12 hours with Raji cells stably expressing GFP and firefly luciferase at an E:T ratio of 2:1. Specific lysis of Raji target cells was determined by detection of firefly luminescence (FIG. 21A). % Specific lysis was defined as 1−[CAR condition luminescence]/[mock condition luminescence]. IFNγ transcript induction was measured 24 hours after electroporation (FIG. 21B).

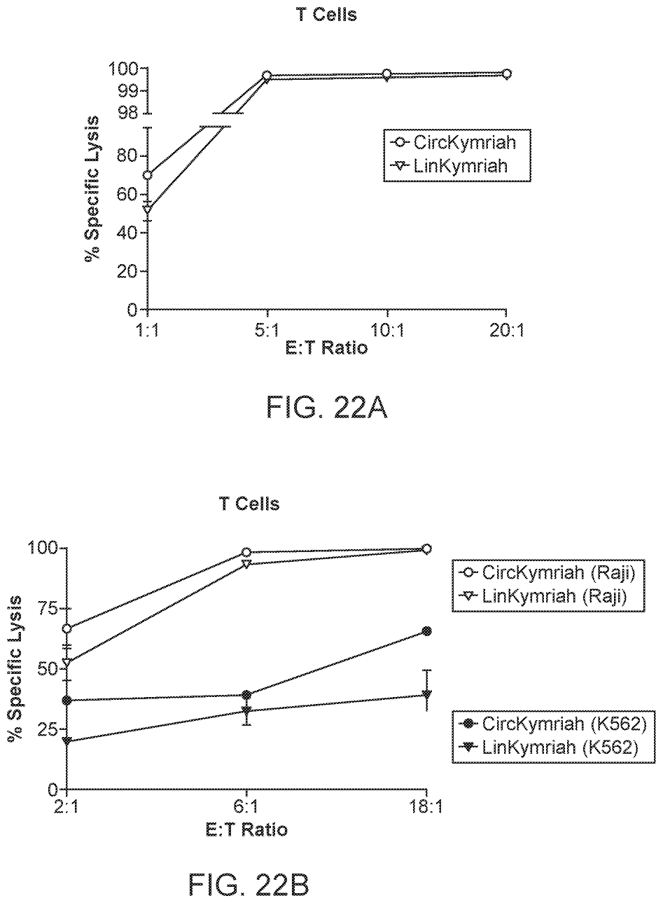

150,000 human primary CD3+ T cells were either mock electroporated or electroporated with 500 ng circRNA or m1ψ-mRNA encoding an anti-CD19 CAR sequence, then co-cultured for 24 hours with Raji cells stably expressing firefly luciferase at different E:T ratios. % Specific lysis of Raji target cells was determined by detection of firefly luminescence (FIG. 22A). % Specific lysis was defined as 1−[CAR condition luminescence]/[mock condition luminescence].

CAR expressing T cells were also co-cultured for 24 hours with Raji or K562 cells stably expressing firefly luciferase at different E:T ratios. Specific lysis of Raji target cells or K562 non-target cells was determined by detection of firefly luminescence (FIG. 22B). % Specific lysis is defined as 1−[CAR condition luminescence]/[mock condition luminescence].

### Example 21

Specific Lysis of Target Cells by T Cells Electroporated with Circular RNA or Linear RNA Encoding a CAR.

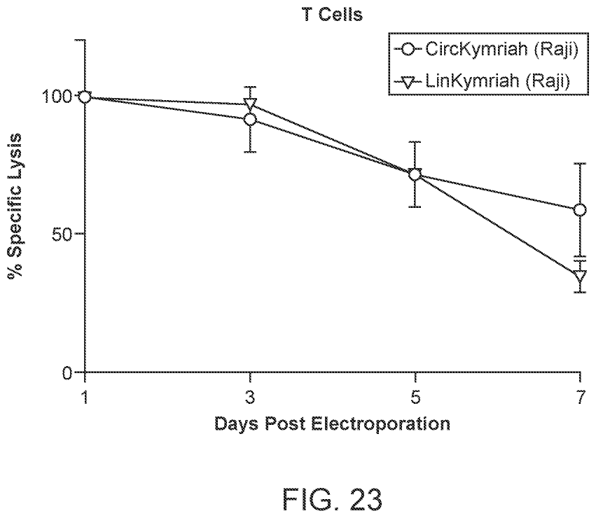

Constructs including Anabaena intron/exon regions, an anti-CD19 CAR expression sequence, and a CVB3 IRES were circularized and reaction products were purified by size exclusion HPLC. Human primary CD3+ T cells were electroporated with 500 ng of circular RNA or an equimolar quantity of m1ψ-mRNA, each encoding a CD19-targeted CAR. Raji cells were added to CAR-T cell cultures over 7 days at an E:T ratio of 10:1. % Specific lysis was measured for both constructs at 1, 3, 5, and 7 days (FIG. 23).

### Example 22

### Specific Lysis of Raji Cells by T Cells Expressing an Anti-CD19 CAR or an Anti-BCMA CAR.

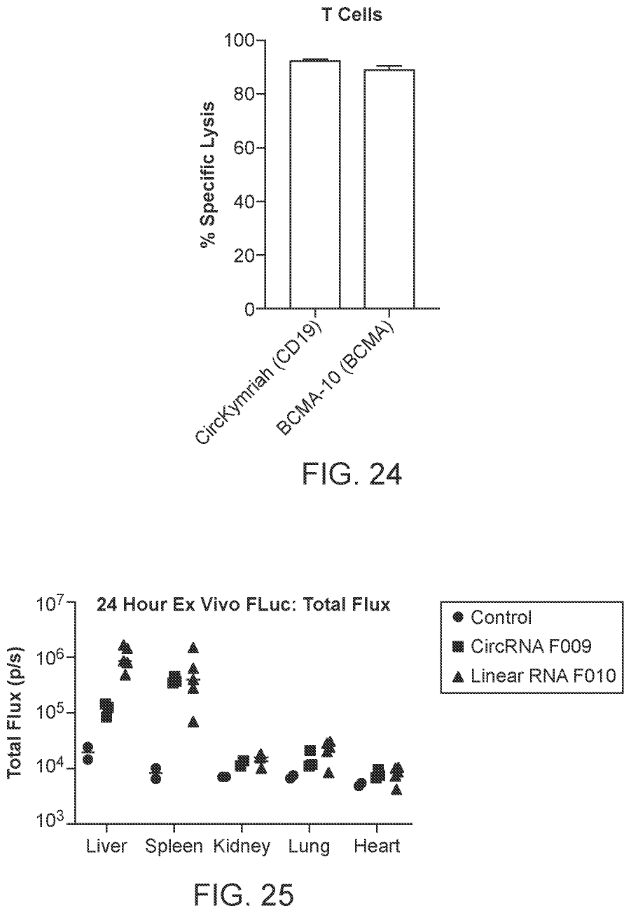

Constructs including Anabaena intron/exon regions, anti-CD19 or anti-BCMA CAR expression sequence, and a CVB3 IRES were circularized and reaction products were purified by size exclusion HPLC. 150,000 primary human CD3+ T cells were electroporated with 500 ng of circRNA, then were co-cultured with Raji cells at an E:T ratio of 2:1. % Specific lysis was measured 12 hours after electroporation (FIG. 24).

### Example 23

### Example 23A: Synthesis of Compounds

Synthesis of representative ionizable lipids of the invention are described in PCT applications PCT/US2016/052352, PCT/US2016/068300, PCT/US2010/061058, PCT/US2018/058555, PCT/US2018/053569, PCT/US2017/028981, PCT/US2019/025246, PCT/US2018/035419, PCT/US2019/015913, and US applications with publication numbers 20190314524, 20190321489, and 20190314284, the contents of each of which are incorporated herein by reference in their entireties.

### Example 23B: Synthesis of Compounds

Synthesis of representative ionizable lipids of the invention are described in US patent publication number US20170210697A1, the contents of which is incorporated herein by reference in its entirety.

### Example 24

### Protein Expression by Organ

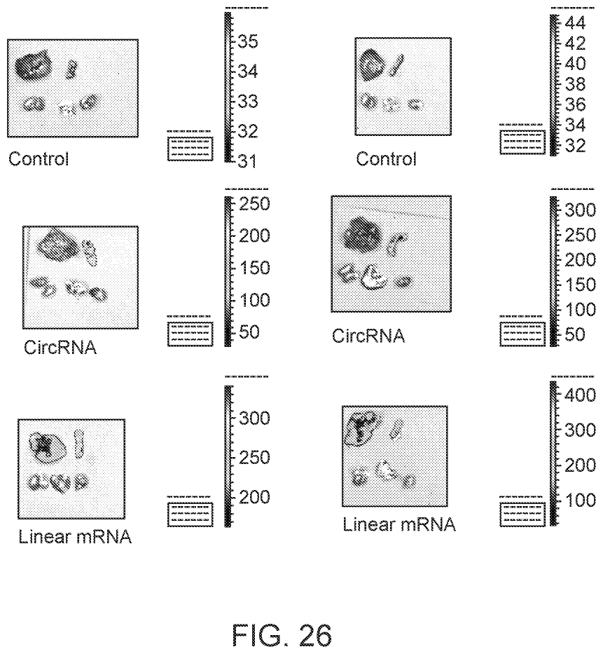

Circular or linear RNA encoding FLuc was generated and loaded into transfer vehicles with the following formulation: 50% ionizable lipid 15 in Table 10b, 10% DSPC, 1.5% PEG-DMG, 38.5% cholesterol. CD-1 mice were dosed at 0.2 mg/kg and luminescence was measured at 6 hours (live IVIS) and 24 hours (live IVIS and ex vivo IVIS). Total Flux (photons/second over a region of interest) of the liver, spleen, kidney, lung, and heart was measured (FIGS. 25 and 26).

### Example 25

### Distribution of Expression in the Spleen

Circular or linear RNA encoding GFP is generated and loaded into transfer vehicles with the following formulation: 50% ionizable lipid 15 in Table 10b, 10% DSPC, 1.5% PEG-DMG, 38.5% cholesterol. The formulation is administered to CD-1 mice. Flow cytometry is run on spleen cells to determine the distribution of expression across cell types.

### Example 26

### Production of Nanoparticle Compositions

In order to investigate safe and efficacious nanoparticle compositions for use in the delivery of circular RNA to cells, a range of formulations are prepared and tested. Specifically, the particular elements and ratios thereof in the lipid component of nanoparticle compositions are optimized.

Nanoparticles can be made in a 1 fluid stream or with mixing processes such as microfluidics and T-junction mixing of two fluid streams, one of which contains the circular RNA and the other has the lipid components.

Lipid compositions are prepared by combining an ionizable lipid, optionally a helper lipid (such as DOPE, DSPC, or oleic acid obtainable from Avanti Polar Lipids, Alabaster, Ala.), a PEG lipid (such as 1,2-dimyristoyl-sn-glycerol methoxypolyethylene glycol, also known as PEG-DMG, obtainable from Avanti Polar Lipids, Alabaster, Ala.), and a structural lipid such as cholesterol at concentrations of about, e.g., 40 or 50 mM in a solvent, e.g., ethanol. Solutions should be refrigerated for storage at, for example, −20° C. Lipids are combined to yield desired molar ratios (see, for example, Tables 31a and 31b below) and diluted with water and ethanol to a final lipid concentration of e.g., between about 5.5 mM and about 25 mM.

TABLE 31a

| Formulation | |
|---|---|
| number | Description |
| 1 | Aliquots of 50 mg/mL ethanolic solutions of C12-200, DOPE, Chol and DMG- |
| | PEG2K (40:30:25:5) are mixed and diluted with ethanol to 3 mL final volume. |
| | Separately, an aqueous buffered solution (10 mM citrate/150 mM NaCl, pH 4.5) of |
| | circRNA is prepared from a 1 mg/mL stock. The lipid solution is injected rapidly |
| | into the aqueous circRNA solution and shaken to yield a final suspension in 20% |
| | ethanol. The resulting nanoparticle suspension is filtered, diafiltrated with 1 × PBS |
| | (pH 7.4), concentrated and stored at 2-8° C. |
| 2 | Aliquots of 50 mg/mL ethanolic solutions of DODAP, DOPE, cholesterol and |
| | DMG-PEG2K (18:56:20:6) are mixed and diluted with ethanol to 3 mL final |
| | volume. Separately, an aqueous buffered solution (10 mM citrate/150 mM NaCl, |
| | pH 4.5) of EPO circRNA is prepared from a 1 mg/mL stock. The lipid solution is |
| | injected rapidly into the aqueous circRNA solution and shaken to yield a final |
| | suspension in 20% ethanol. The resulting nanoparticle suspension is filtered, |
| | diafiltrated with 1 × PBS (pH 7.4), concentrated and stored at 2-8° C. Final |
| | concentration = 1.35 mg/mL EPO circRNA (encapsulated). Zave = 75.9 nm |
| | (Dv(50) = 57.3 nm; Dv(90) = 92.1 nm). |
| 3 | Aliquots of 50 mg/mL ethanolic solutions of HGT4003, DOPE, cholesterol and |
| | DMG-PEG2K (50:25:20:5) are mixed and diluted with ethanol to 3 mL final |
| | volume. Separately, an aqueous buffered solution (10 mM citrate/150 mM NaCl, |
| | pH 4.5) of circRNA is prepared from a 1 mg/mL stock. The lipid solution is |
| | injected rapidly into the aqueous circRNA solution and shaken to yield a final |
| | suspension in 20% ethanol. The resulting nanoparticle suspension is filtered, |
| | diafiltrated with 1 × PBS (pH 7.4), concentrated and stored at 2-8° C. |
| 4 | Aliquots of 50 mg/mL ethanolic solutions of ICE, DOPE and DMG-PEG2K |
| | (70:25:5) are mixed and diluted with ethanol to 3 mL final volume. Separately, an |
| | aqueous buffered solution (10 mM citrate/150 mM NaCl, pH 4.5) of circRNA is |
| | prepared from a 1 mg/mL stock. The lipid solution is injected rapidly into the |
| | aqueous circRNA solution and shaken to yield a final suspension in 20% ethanol. |
| | The resulting nanoparticle suspension is filtered, diafiltrated with 1 × PBS (pH 7.4), |
| | concentrated and stored at 2-8° C. |
| 5 | Aliquots of 50 mg/mL ethanolic solutions of HGT5000, DOPE, cholesterol and |
| | DMG-PEG2K (40:20:35:5) are mixed and diluted with ethanol to 3 mL final |
| | volume. Separately, an aqueous buffered solution (10 mM citrate/150 mM NaCl, |
| | pH 4.5) of EPO circRNA is prepared from a 1 mg/mL stock. The lipid solution is |
| | injected rapidly into the aqueous circRNA solution and shaken to yield a final |
| | suspension in 20% ethanol. The resulting nanoparticle suspension is filtered, |
| | diafiltrated with 1 × PBS (pH 7.4), concentrated and stored at 2-8° C. Final |
| | concentration = 1.82 mg/mL EPO mRNA (encapsulated). Zave = 105.6 nm |
| | (Dv(50) = 53.7 nm; Dv(90) = 157 nm). |
| 6 | Aliquots of 50 mg/mL ethanolic solutions of HGT5001, DOPE, cholesterol and |
| | DMG-PEG2K (40:20:35:5) are mixed and diluted with ethanol to 3 mL final |
| | volume. Separately, an aqueous buffered solution (10 mM citrate/150 mM NaCl, |
| | pH 4.5) of EPO circRNA is prepared from a 1 mg/mL stock. The lipid solution is |
| | injected rapidly into the aqueous circRNA solution and shaken to yield a final |
| | suspension in 20% ethanol. The resulting nanoparticle suspension is filtered, |
| | diafiltrated with 1 × PBS (pH 7.4), concentrated and stored at 2-8° C. |
| 7 | Aliquots of 50 mg/mL ethanolic solutions of HGT5001, DOPE, cholesterol and |
| | DMG-PEG2K (35:16:46.5:2.5) are mixed and diluted with ethanol to 3 mL final |
| | volume. Separately, an aqueous buffered solution (10 mM citrate/150 mM NaCl, |
| | pH 4.5) of EPO circRNA is prepared from a 1 mg/mL stock. The lipid solution is |
| | injected rapidly into the aqueous circRNA solution and shaken to yield a final |
| | suspension in 20% ethanol. The resulting nanoparticle suspension is filtered, |
| | diafiltrated with 1 × PBS (pH 7.4), concentrated and stored at 2-8° C. |
| 8 | Aliquots of 50 mg/mL ethanolic solutions of HGT5001, DOPE, cholesterol and |
| | DMG-PEG2K (40:10:40:10) are mixed and diluted with ethanol to 3 mL final |
| | volume. Separately, an aqueous buffered solution (10 mM citrate/150 mM NaCl, |
| | pH 4.5) of EPO circRNA is prepared from a 1 mg/mL stock. The lipid solution is |
| | injected rapidly into the aqueous circRNA solution and shaken to yield a final |
| | suspension in 20% ethanol. The resulting nanoparticle suspension is filtered, |
| | diafiltrated with 1 × PBS (pH 7.4), concentrated and stored at 2-8° C. |

In some embodiments, transfer vehicle has a formulation as described in Table 31a.

TABLE 31b

| Composition (mol %) | Components |
|---|---|
| 40:20:38.5:1.5 | Compound:Phospholipid:Phytosterol*:PEG-DMG |
| 45:15:38.5:1.5 | Compound:Phospholipid:Phytosterol*:PEG-DMG |
| 50:10:38.5:1.5 | Compound:Phospholipid:Phytosterol*:PEG-DMG |
| 55:5:38.5:1.5 | Compound:Phospholipid:Phytosterol*:PEG-DMG |
| 60:5:33.5:1.5 | Compound:Phospholipid:Phytosterol*:PEG-DMG |
| 45:20:33.5:1.5 | Compound:Phospholipid:Phytosterol*:PEG-DMG |
| 50:20:28.5:1.5 | Compound:Phospholipid:Phytosterol*:PEG-DMG |
| 55:20:23.5:1.5 | Compound:Phospholipid:Phytosterol*:PEG-DMG |
| 60:20:18.5:1.5 | Compound:Phospholipid:Phytosterol*:PEG-DMG |
| 40:15:43.5:1.5 | Compound:Phospholipid:Phytosterol*:PEG-DMG |
| 50:15:33.5:1.5 | Compound:Phospholipid:Phytosterol*:PEG-DMG |
| 55:15:28.5:1.5 | Compound:Phospholipid:Phytosterol*:PEG-DMG |
| 60:15:23.5:1.5 | Compound:Phospholipid:Phytosterol*:PEG-DMG |
| 40:10:48.5:1.5 | Compound:Phospholipid:Phytosterol*:PEG-DMG |
| 45:10:43.5:1.5 | Compound:Phospholipid:Phytosterol*:PEG-DMG |
| 55:10:33.5:1.5 | Compound:Phospholipid:Phytosterol*:PEG-DMG |
| 60:10:28.5:1.5 | Compound:Phospholipid:Phytosterol*:PEG-DMG |
| 40:5:53.5:1.5 | Compound:Phospholipid:Phytosterol*:PEG-DMG |
| 45:5:48.5:1.5 | Compound:Phospholipid:Phytosterol*:PEG-DMG |
| 50:5:43.5:1.5 | Compound:Phospholipid:Phytosterol*:PEG-DMG |
| 40:20:40:0 | Compound:Phospholipid:Phytosterol*:PEG-DMG |
| 45:20:35:0 | Compound:Phospholipid:Phytosterol*:PEG-DMG |
| 50:20:30:0 | Compound:Phospholipid:Phytosterol*:PEG-DMG |
| 55:20:25:0 | Compound:Phospholipid:Phytosterol*:PEG-DMG |
| 60:20:20:0 | Compound:Phospholipid:Phytosterol*:PEG-DMG |
| 40:15:45:0 | Compound:Phospholipid:Phytosterol*:PEG-DMG |

In some embodiments, transfer vehicle has a formulation as described in Table 31b.

For nanoparticle compositions including circRNA, solutions of the circRNA at concentrations of 0.1 mg/ml in deionized water are diluted in a buffer, e.g., 50 mM sodium citrate buffer at a pH between 3 and 4 to form a stock solution. Alternatively, solutions of the circRNA at concentrations of 0.15 mg/ml in deionized water are diluted in a buffer, e.g., 6.25 mM sodium acetate buffer at a pH between 3 and 4.5 to form a stock solution.

Nanoparticle compositions including a circular RNA and a lipid component are prepared by combining the lipid solution with a solution including the circular RNA at lipid component to circRNA wt:wt ratios between about 5:1 and about 50:1. The lipid solution is rapidly injected using, e.g., a NanoAssemblr microfluidic based system at flow rates between about 10 ml/min and about 18 ml/min or between about 5 ml/min and about 18 ml/min into the circRNA solution, to produce a suspension with a water to ethanol ratio between about 1:1 and about 4:1.

Nanoparticle compositions can be processed by dialysis to remove ethanol and achieve buffer exchange. Formulations are dialyzed twice against phosphate buffered saline (PBS), pH 7.4, at volumes 200 times that of the primary product using Slide-A-Lyzer cassettes (Thermo Fisher Scientific Inc., Rockford, Ill.) with a molecular weight cutoff of 10 kDa or 20 kDa. The formulations are then dialyzed overnight at 4° C. The resulting nanoparticle suspension is filtered through 0.2 μm sterile filters (Sarstedt, Nümbrecht, Germany) into glass vials and sealed with crimp closures. Nanoparticle composition solutions of 0.01 mg/ml to 0.15 mg/ml are generally obtained.

The method described above induces nano-precipitation and particle formation.

Alternative processes including, but not limited to, T-junction and direct injection, may be used to achieve the same nano-precipitation. B. Characterization of nanoparticle compositions

A Zetasizer Nano ZS (Malvern Instruments Ltd, Malvern, Worcestershire, UK) can be used to determine the particle size, the polydispersity index (PDI) and the zeta potential of the nanoparticle compositions in 1×PBS in determining particle size and 15 mM PBS in determining zeta potential.

Ultraviolet-visible spectroscopy can be used to determine the concentration of circRNA in nanoparticle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, Calif.). The concentration of circRNA in the nanoparticle composition can be calculated based on the extinction coefficient of the circRNA used in the composition and on the difference between the absorbance at a wavelength of, for example, 260 nm and the baseline value at a wavelength of, for example, 330 nm.

A QUANT-IT™ RIBOGREEN® RNA assay (Invitrogen Corporation Carlsbad, Calif.) can be used to evaluate the encapsulation of circRNA by the nanoparticle composition. The samples are diluted to a concentration of approximately 5 μg/mL or 1 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2-4% Triton X-100 solution is added to the wells. The plate is incubated at a temperature of 37° C. for 15 minutes. The RIBOGREEN® reagent is diluted 1:100 or 1:200 in TE buffer, and 100 μL of this solution is added to each well. The fluorescence intensity can be measured using a fluorescence plate reader (Wallac Victor 1420 Multilabel Counter; Perkin Elmer, Waltham, Mass.) at an excitation wavelength of, for example, about 480 nm and an emission wavelength of, for example, about 520 nm. The fluorescence values of the reagent blank are subtracted from that of each of the samples and the percentage of free circRNA is determined by dividing the fluorescence intensity of the intact sample (without addition of Triton X-100) by the fluorescence value of the disrupted sample (caused by the addition of Triton X-100). C.

### In Vivo Formulation Studies:

In order to monitor how effectively various nanoparticle compositions deliver circRNA to targeted cells, different nanoparticle compositions including circRNA are prepared and administered to rodent populations. Mice are intravenously, intramuscularly, intraarterially, or intratumorally administered a single dose including a nanoparticle composition with a lipid nanoparticle formulation. In some instances, mice may be made to inhale doses. Dose sizes may range from 0.001 mg/kg to 10 mg/kg, where 10 mg/kg describes a dose including 10 mg of a circRNA in a nanoparticle composition for each 1 kg of body mass of the mouse. A control composition including PBS may also be employed.

Upon administration of nanoparticle compositions to mice, dose delivery profiles, dose responses, and toxicity of particular formulations and doses thereof can be measured by enzyme-linked immunosorbent assays (ELISA), bioluminescent imaging, or other methods. Time courses of protein expression can also be evaluated. Samples collected from the rodents for evaluation may include blood and tissue (for example, muscle tissue from the site of an intramuscular injection and internal tissue); sample collection may involve sacrifice of the animals.

Higher levels of protein expression induced by administration of a composition including a circRNA will be indicative of higher circRNA translation and/or nanoparticle composition circRNA delivery efficiencies. As the non-RNA components are not thought to affect translational machineries themselves, a higher level of protein expression is likely indicative of a higher efficiency of delivery of the circRNA by a given nanoparticle composition relative to other nanoparticle compositions or the absence thereof.

### Example 27

### Characterization of Nanoparticle Compositions

A Zetasizer Nano ZS (Malvern Instruments Ltd, Malvern, Worcestershire, UK) can be used to determine the particle size, the polydispersity index (PDI) and the zeta potential of the transfer vehicle compositions in 1×PBS in determining particle size and 15 mM PBS in determining zeta potential.

Ultraviolet-visible spectroscopy can be used to determine the concentration of a therapeutic and/or prophylactic (e.g., RNA) in transfer vehicle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, Calif.). The concentration of therapeutic and/or prophylactic in the transfer vehicle composition can be calculated based on the extinction coefficient of the therapeutic and/or prophylactic used in the composition and on the difference between the absorbance at a wavelength of, for example, 260 nm and the baseline value at a wavelength of, for example, 330 nm.

For transfer vehicle compositions including RNA, a QUANT-IT™ RIBOGREEN® RNA assay (Invitrogen Corporation Carlsbad, Calif.) can be used to evaluate the encapsulation of RNA by the transfer vehicle composition. The samples are diluted to a concentration of approximately 5 μg/mL or 1 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2-4% Triton X-100 solution is added to the wells. The plate is incubated at a temperature of 37° C. for 15 minutes. The RIBOGREEN® reagent is diluted 1:100 or 1:200 in TE buffer, and 100 μL of this solution is added to each well. The fluorescence intensity can be measured using a fluorescence plate reader (Wallac Victor 1420 Multilabel Counter; Perkin Elmer, Waltham, Mass.) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

### Example 28

### T Cell Targeting

To target transfer vehicles to T-cells, T cell antigen binders, e.g., anti-CD8 antibodies, are coupled to the surface of the transfer vehicle. Anti-T cell antigen antibodies are mildly reduced with an excess of DTT in the presence of EDTA in PBS to expose free hinge region thiols. To remove DTT, antibodies are passed through a desalting column. The heterobifunctional cross-linker SM(PEG)24 is used to anchor antibodies to the surface of circRNA-loaded transfer vehicles (Amine groups are present in the head groups of PEG lipids, free thiol groups on antibodies were created by DTT, SM(PEG)24 cross-links between amines and thiol groups). Transfer vehicles are first incubated with an excess of SM(PEG)24 and centrifuged to remove unreacted cross-linker. Activated transfer vehicles are then incubated with an excess of reduced anti-T cell antigen antibody. Unbound antibody is removed using a centrifugal filtration device.

### Example 29

### RNA Containing Transfer Vehicle Using RV88.

In this example RNA containing transfer vehicles are synthesized using the 2-D vortex microfluidic chip with the cationic lipid RV88 for delivery of circRNA.

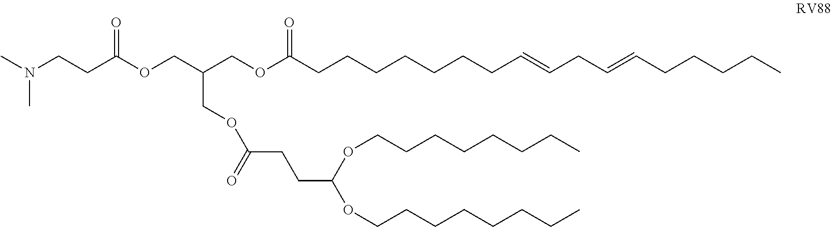 [SMILES: CCCCC/C=C/C/C=C/CCCCCCCC(=O)OCC(COC(=O)CCC(OCCCCCCCC)OCCCCCCCC)COC(=O)CCN(C)C]

TABLE 32a

| Materials and Instrument | Vendor | Cat # |
|---|---|---|
| 1M Tris-HCl, pH 8.0, Sterile | Teknova | T1080 |
| 5M Sodium Chloride solution | Teknova | S0250 |
| QB Citrate buffer, pH 6.0 | Teknova | Q2446 |
| (100 mM) | | |
| Nuclease-free water | Ambion | AM9937 |
| Triton X-100 | Sigma-Aldrich | T8787-100ML |
| RV88 | GVK bio | |
| DSPC | Lipoid | 556500 |
| Cholesterol | Sigma | C3045-5G |
| PEG2K | Avanti Polar Lipids | 880150 |
| Ethanol | Acros Organic | 615090010 |
| 5 mL Borosilicate glass vials | Thermo Scientific | ST5-20 |
| PD MiniTrap G-25 Desalting | GE Healthcare | VWR Cat. |
| Columns | | #95055-984 |
| Quant-IT RiboGreen RNA | Molecular Probes/ | R11490 |
| Assay kit | Life Technologies | |
| Black 96-well microplates | Greiner | 655900 |

RV88, DSPC, and cholesterol all being prepared in ethanol at a concentration of 10 mg/ml in borosilica vials. The lipid 14:0-PEG2K PE is prepared at a concentration of 4 mg/ml also in a borosilica glass vial. Dissolution of lipids at stock concentrations is attained by sonication of the lipids in ethanol for 2 min. The solutions are then heated on an orbital tilting shaker set at 170 rpm at 37° C. for 10 min. Vials are then equilibrated at 26° C. for a minimum of 45 min. The lipids are then mixed by adding volumes of stock lipid as shown in Table 32b. The solution is then adjusted with ethanol such that the final lipid concentration was 7.92 mg/ml.

TABLE 32b

| | | | | | Stock | | Ethanol |
|---|---|---|---|---|---|---|---|
| Composition | MW | % | nmoles | mg | (mg/ml) | ul | (ul) |
| RV86 | 794.2 | 40% | 7200 | 5.72 | 10 | 571.8 | 155.3 |
| DSPC | 790.15 | 10% | 1800 | 1.42 | 10 | 142.2 | |
| Cholesterol | 386.67 | 48% | 8640 | 3.34 | 10 | 334.1 | |
| PEG2K | 2693.3 | 2% | 360 | 0.97 | 4 | 242.4 | |

RNA is prepared as a stock solution with 75 mM Citrate buffer at pH 6.0 and a concentration of RNA at 1.250 mg/ml. The concentration of the RNA is then adjusted to 0.1037 mg/ml with 75 mM citrate buffer at pH 6.0, equilibrated to 26° C. The solution is then incubated at 26° C. for a minimum of 25 min.

The microfluidic chamber is cleaned with ethanol and neMYSIS syringe pumps are prepared by loading a syringe with the RNA solution and another syringe with the ethanolic lipid. Both syringes are loaded and under the control of neMESYS software. The solutions are then applied to the mixing chip at an aqueous to organic phase ratio of 2 and a total flow rate of 22 ml/min (14.67 ml/min for RNA and 7.33 ml/min for the lipid solution. Both pumps are started synchronously. The mixer solution that flowed from the microfluidic chip is collected in 4×1 ml fractions with the first fraction being discarded as waste. The remaining solution containing the RNA-liposomes is exchanged by using G-25 mini desalting columns to 10 mM Tris-HCl, 1 mM EDTA, at pH 7.5. Following buffer exchange, the materials are characterized for size, and RNA entrapment through DLS analysis and Ribogreen assays, respectively.

### Example 30

### RNA Containing Transfer Vehicle Using RV94.

In this example, RNA containing liposome are synthesized using the 2-D vortex microfluidic chip with the cationic lipid RV94 for delivery of circRNA.

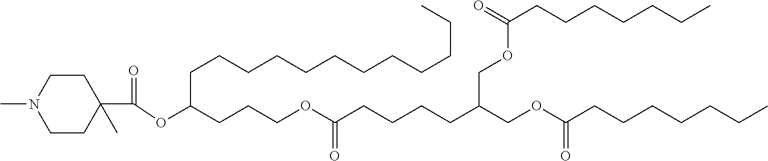 [SMILES: CCCCCCCCCCCCC(CCCOC(=O)CCCCC(COC(=O)CCCCCCC)COC(=O)CCCCCCC)OC(=O)C1(C)CCN(C)CC1]

TABLE 33

| Materials and Instrument | Vendor | Cat # |
|---|---|---|
| 1M Tris-HCl, pH 8.0, Sterile | Teknova | T1080 |
| 5M Sodium Chloride solution | Teknova | S0250 |
| QB Citrate buffer, pH 6.0 | Teknova | Q2446 |
| (100 mM) | | |
| Nuclease-free water | Ambion | AM9937 |
| Triton X-100 | Sigma-Aldrich | T8787-100ML |
| RV94 | GVKbio | |
| DSPC | Lipoid | 556500 |
| Cholesterol | Sigma | C3045-5G |
| PEG2K | Avanti Polar Lipids | 880150 |
| Ethanol | Acros Organic | 615090010 |
| 5 mL Borosilicate glass vials | Thermo Scientific | ST5-20 |
| PD MiniTrap G-25 Desalting | GE Healthcare | VWR Cat. |
| Columns | | #95055-984 |
| Quant-IT RiboGreen RNA | Molecular Probes/ | R11490 |
| Assay kit | Life Technologies | |
| Black 96-well microplates | Greiner | 655900 |

The lipids were prepared as in Example 29 using the material amounts named in Table 34 to a final lipid concentration of 7.92 mg/ml.

TABLE 34

| | | | | | Stock | | Ethanol |
|---|---|---|---|---|---|---|---|
| Composition | MW | % | nmoles | mg | (mg/ml) | ul | (ul) |
| RV94 | 808.22 | 40% | 2880 | 2.33 | 10 | 232.8 | 155.3 |
| DSPC | 790.15 | 10% | 720 | 0.57 | 10 | 56.9 | |
| Cholesterol | 386.67 | 48% | 3456 | 1.34 | 10 | 133.6 | |
| PEG2K | 2693.3 | 2% | 144 | 0.39 | 4 | 97.0 | |

The aqueous solution of circRNA is prepared as a stock solution with 75 mM Citrate buffer at pH 6.0 the circRNA at 1.250 mg/ml. The concentration of the RNA is then adjusted to 0.1037 mg/ml with 75 mM citrate buffer at pH 6.0, equilibrated to 26° C. The solution is then incubated at 26° C. for a minimum of 25 min.

The microfluidic chamber is cleaned with ethanol and neMYSIS syringe pumps are prepared by loading a syringe with the RNA solution and another syringe with the ethanolic lipid. Both syringes are loaded and under the control of neMESYS software. The solutions are then applied to the mixing chip at an aqueous to organic phase ratio of 2 and a total flow rate of 22 ml/min (14.67 ml/min for RNA and 7.33 ml/min for the lipid solution. Both pumps are started synchronously. The mixer solution that flowed from the microfluidic chip is collected in 4×1 ml fractions with the first fraction being discarded as waste. The remaining solution containing the circRNA-transfer vehicles is exchanged by using G-25 mini desalting columns to 10 mM Tris-HCl, 1 mM EDTA, at pH 7.5, as described above. Following buffer exchange, the materials are characterized for size, and RNA entrapment through DLS analysis and Ribogreen assays, respectively. The biophysical analysis of the liposomes is shown in Table 35.

TABLE 35

| | | | Ratio | | RNA encap- | | |
|---|---|---|---|---|---|---|---|
| Sample | N:P | TFR | (aqueous/ | RNA encap- | sulation yield | size | |
| Name | Ratio | ml/min | org phase) | sulation amount | % | d · nm | PDI |
| SAM- | 8 | 22 | 2 | 31.46 | 86.9 | 113.1 | 0.12 |
| RV94 | | | | | | | |

### Example 31

### General Protocol for in Line Mixing.

Individual and separate stock solutions are prepared—one containing lipid and the other circRNA. Lipid stock containing a desired lipid or lipid mixture, DSPC, cholesterol and PEG lipid is prepared by solubilized in 90% ethanol. The remaining 10% is low pH citrate buffer. The concentration of the lipid stock is 4 mg/mL. The pH of this citrate buffer can range between pH 3 and pH 5, depending on the type of lipid employed. The circRNA is also solubilized in citrate buffer at a concentration of 4 mg/mL. 5 mL of each stock solution is prepared.

Stock solutions are completely clear and lipids are ensured to be completely solubilized before combining with circRNA. Stock solutions may be heated to completely solubilize the lipids. The circRNAs used in the process may be unmodified or modified oligonucleotides and may be conjugated with lipophilic moieties such as cholesterol.

The individual stocks are combined by pumping each solution to a T-junction. A dual-head Watson-Marlow pump was used to simultaneously control the start and stop of the two streams. A 1.6 mm polypropylene tubing is further downsized to 0.8 mm tubing in order to increase the linear flow rate. The polypropylene line (ID=0.8 mm) are attached to either side of a T-junction. The polypropylene T has a linear edge of 1.6 mm for a resultant volume of 4.1 mm³. Each of the large ends (1.6 mm) of polypropylene line is placed into test tubes containing either solubilized lipid stock or solubilized circRNA. After the T-junction, a single tubing is placed where the combined stream exited. The tubing is then extended into a container with 2× volume of PBS, which is rapidly stirred. The flow rate for the pump is at a setting of 300 rpm or 110 mL/min. Ethanol is removed and exchanged for PBS by dialysis. The lipid formulations are then concentrated using centrifugation or diafiltration to an appropriate working concentration.

C57BL/6 mice (Charles River Labs, MA) receive either saline or formulated circRNA via tail vein injection. At various time points after administration, serum samples are collected by retroorbital bleed. Serum levels of Factor VII protein are determined in samples using a chromogenic assay (Biophen FVTI, Aniara Corporation, OH). To determine liver RNA levels of Factor VII, animals are sacrificed and livers are harvested and snap frozen in liquid nitrogen. Tissue lysates are prepared from the frozen tissues and liver RNA levels of Factor VII are quantified using a branched DNA assay (QuantiGene Assay, Panomics, Calif.).

FVII activity is evaluated in FVTI siRNA-treated animals at 48 hours after intravenous (bolus) injection in C57BL/6 mice. FVII is measured using a commercially available kit for determining protein levels in serum or tissue, following the manufacturer's instructions at a microplate scale. FVII reduction is determined against untreated control mice, and the results are expressed as % Residual FVII. Two dose levels (0.05 and 0.005 mg/kg FVII siRNA) are used in the screen of each novel liposome composition.

### Example 32

circRNA Formulation Using Preformed Vesicles.

Cationic lipid containing transfer vehicles are made using the preformed vesicle method. Cationic lipid, DSPC, cholesterol and PEG-lipid are solubilized in ethanol at a molar ratio of 40/10/40/10, respectively. The lipid mixture is added to an aqueous buffer (50 mM citrate, pH 4) with mixing to a final ethanol and lipid concentration of 30% (vol/vol) and 6.1 mg/mL respectively and allowed to equilibrate at room temperature for 2 min before extrusion. The hydrated lipids are extruded through two stacked 80 nm pore-sized filters (Nuclepore) at 22° C. using a Lipex Extruder (Northern Lipids, Vancouver, BC) until a vesicle diameter of 70-90 nm, as determined by Nicomp analysis, is obtained. For cationic lipid mixtures which do not form small vesicles, hydrating the lipid mixture with a lower pH buffer (50 mM citrate, pH 3) to protonate the phosphate group on the DSPC headgroup helps form stable 70-90 nm vesicles.

The FVII circRNA (solubilised in a 50 mM citrate, pH 4 aqueous solution containing 30% ethanol) is added to the vesicles, pre-equilibrated to 35° C., at a rate of ˜5 mL/min with mixing. After a final target circRNA/lipid ratio of 0.06 (wt wt) is achieved, the mixture is incubated for a further 30 min at 35° C. to allow vesicle re-organization and encapsulation of the FVII RNA. The ethanol is then removed and the external buffer replaced with PBS (155 mM NaCl, 3 mM Na2HPO4, ImM KH2PO4, pH 7.5) by either dialysis or tangential flow diafiltration. The final encapsulated circRNA-to-lipid ratio is determined after removal of unencapsulated RNA using size-exclusion spin columns or ion exchange spin columns.

### Example 33

Expression of Trispecific Antigen Binding Proteins from Engineered Circular RNA

Circular RNAs are designed to include: (1) a 3′ post splicing group I intron fragment; (2) an Internal Ribosome Entry Site (IRES); (3) a trispecific antigen-binding protein coding region; and (4) a 3′ homology region. The trispecific antigen-binding protein regions are constructed to produce an exemplary trispecific antigen-binding protein that will bind to a target antigen, e.g., GPC3.

Generation of a scFv CD3 Binding Domain

The human CD3epsilon chain canonical sequence is Uniprot Accession No. P07766. The human CD3gamma chain canonical sequence is Uniprot Accession No. P09693. The human CD3delta chain canonical sequence is Uniprot Accession No. P043234. Antibodies against CD3epsilon, CD3gamma or CD3delta are generated via known technologies such as affinity maturation. Where murine anti-CD3 antibodies are used as a starting material, humanization of murine anti-CD3 antibodies is desired for the clinical setting, where the mouse-specific residues may induce a human-anti-mouse antigen (HAMA) response in subjects who receive treatment of a trispecific antigen-binding protein described herein. Humanization is accomplished by grafting CDR regions from murine anti-CD3 antibody onto appropriate human germline acceptor frameworks, optionally including other modifications to CDR and/or framework regions.

Human or humanized anti-CD3 antibodies are therefore used to generate scFv sequences for CD3 binding domains of a trispecific antigen-binding protein. DNA sequences coding for human or humanized VL and VH domains are obtained, and the codons for the constructs are, optionally, optimized for expression in cells from Homo sapiens. The order in which the VL and VH domains appear in the scFv is varied (i.e. VL-VH, or VH-VL orientation), and three copies of the “G4S” or “G₄S” subunit (G₄S)₃ connect the variable domains to create the scFv domain. Anti-CD3 scFv plasmid constructs can have optional Flag, His or other affinity tags, and are electroporated into HEK293 or other suitable human or mammalian cell lines and purified. Validation assays include binding analysis by FACS, kinetic analysis using Proteon, and staining of CD3-expressing cells.

Generation of a scFv Glypican-3 (GPC3) Binding Domain

Glypican-3 (GPC3) is one of the cell surface proteins present on Hepatocellular Carcinoma but not on healthy normal liver tissue. It is frequently observed to be elevated in hepatocellular carcinoma and is associated with poor prognosis for HCC patients. It is known to activate Wnt signalling. GPC3 antibodies have been generated including MDX-1414, HN3, GC33, and YP7.

A scFv binding to GPC-3 or another target antigen is generated similarly to the above method for generation of a scFv binding domain to CD3.

### Expression of Trispecific Antigen-Binding Proteins In Vitro

A CHO cell expression system (Flp-In®, Life Technologies), a derivative of CHO-K1 Chinese Hamster ovary cells (ATCC, CCL-61) (Kao and Puck, Proc. Natl. Acad Sci USA 1968; 60(4):1275-81), is used. Adherent cells are subcultured according to standard cell culture protocols provided by Life Technologies.

For adaption to growth in suspension, cells are detached from tissue culture flasks and placed in serum-free medium. Suspension-adapted cells are cryopreserved in medium with 10% DMSO.

Recombinant CHO cell lines stably expressing secreted trispecific antigen-binding proteins are generated by transfection of suspension-adapted cells. During selection with the antibiotic Hygromycin B viable cell densities are measured twice a week, and cells are centrifuged and resuspended in fresh selection medium at a maximal density of 0.1×10⁶viable cells/mL. Cell pools stably expressing trispecific antigen-binding proteins are recovered after 2-3 weeks of selection at which point cells are transferred to standard culture medium in shake flasks. Expression of recombinant secreted proteins is confirmed by performing protein gel electrophoresis or flow cytometry. Stable cell pools are cryopreserved in DMSO containing medium.

Trispecific antigen-binding proteins are produced in 10-day fed-batch cultures of stably transfected CHO cell lines by secretion into the cell culture supernatant. Cell culture supernatants are harvested after 10 days at culture viabilities of typically >75%. Samples are collected from the production cultures every other day and cell density and viability are assessed. On day of harvest, cell culture supernatants are cleared by centrifugation and vacuum filtration before further use.

Protein expression titers and product integrity in cell culture supernatants are analyzed by SDS-PAGE.

### Purification of Trispecific Antigen-Binding Proteins

Trispecific antigen-binding proteins are purified from CHO cell culture supernatants in a two-step procedure. The constructs are subjected to affinity chromatography in a first step followed by preparative size exclusion chromatography (SEC) on Superdex 200 in a second step. Samples are buffer-exchanged and concentrated by ultrafiltration to a typical concentration of >1 mg/mL Purity and homogeneity (typically >90%) of final samples are assessed by SDS PAGE under reducing and non-reducing conditions, followed by immunoblotting using an anti-(half-life extension domain) or anti idiotype antibody as well as by analytical SEC, respectively. Purified proteins are stored at aliquots at −80° C. until use.

### Example 34

Expression of Engineered Circular RNA with a Half-Life Extension Domain has Improved Pharmacokinetic Parameters than without a Half-Life Extension Domain

The trispecific antigen-binding protein encoded on a circRNA molecule of example 23 is administered to cynomolgus monkeys as a 0.5 mg/kg bolus injection intramuscularly. Another cynomolgus monkey group receives a comparable protein encoded on a circRNA molecule in size with binding domains to CD3 and GPC-3, but lacking a half-life extension domain. A third and fourth group receive a protein encoded on a circRNA molecule with CD3 and half-life extension domain binding domains and a protein with GPC-3 and half-life extension domains, respectively. Both proteins encoded by circRNA are comparable in size to the trispecific antigen-binding protein. Each test group consists of 5 monkeys. Serum samples are taken at indicated time points, serially diluted, and the concentration of the proteins is determined using a binding ELISA to CD3 and/or GPC-3.

Pharmacokinetic analysis is performed using the test article plasma concentrations. Group mean plasma data for each test article conforms to a multi-exponential profile when plotted against the time post-dosing. The data are fit by a standard two-compartment model with bolus input and first-order rate constants for distribution and elimination phases. The general equation for the best fit of the data for i.v. administration is: c(t)=Ae^{˜at}+Be^{˜pt}, where c(t) is the plasma concentration at time t, A and B are intercepts on the Y-axis, and α and β are the apparent first-order rate constants for the distribution and elimination phases, respectively. The a-phase is the initial phase of the clearance and reflects distribution of the protein into all extracellular fluid of the animal, whereas the second or β-phase portion of the decay curve represents true plasma clearance. Methods for fitting such equations are well known in the art. For example, A=D/V(a−k21)/(a−p), B=D/V(p−k21)/(a−p), and a and β (for α>β) are roots of the quadratic equation: r²+(k12+k21+k10)r+1(21k10=0 using estimated parameters of V=volume of distribution, k10=elimination rate, k12=transfer rate from compartment 1 to compartment 2 and k21=transfer rate from compartment 2 to compartment 1, and D=the administered dose.

Data analysis: Graphs of concentration versus time profiles are made using KaleidaGraph (KaleidaGraph™ V. 3.09 Copyright 1986-1997. Synergy Software. Reading, Pa.). Values reported as less than reportable (LTR) are not included in the PK analysis and are not represented graphically. Pharmacokinetic parameters are determined by compartmental analysis using WinNonlin software (WinNonlin® Professional V. 3.1 WinNonlin™ Copyright 1998-1999. Pharsight Corporation. Mountain View, Calif.). Pharmacokinetic parameters are computed as described in Ritschel W A and Kearns G L, 1999, EST: Handbook Of Basic Pharmacokinetics Including Clinical Applications, 5th edition, American Pharmaceutical Assoc., Washington, D C.

It is expected that the trispecific antigen-binding protein encoded on a circRNA molecule of Example 23 has improved pharmacokinetic parameters such as an increase in elimination half-time as compared to proteins lacking a half-life extension domain.

### Example 35

### Cytotoxicity of the Trispecific Antigen-Binding Protein

The trispecific antigen-binding protein encoded on a circRNA molecule of Example 23 is evaluated in vitro on its mediation of T cell dependent cytotoxicity to GPC-3+ target cells.

Fluorescence labeled GPC3 target cells are incubated with isolated PBMC of random donors or T-cells as effector cells in the presence of the trispecific antigen-binding protein of Example 23. After incubation for 4 h at 37° C. in a humidified incubator, the release of the fluorescent dye from the target cells into the supernatant is determined in a spectrofluorimeter. Target cells incubated without the trispecific antigen-binding protein of Example 23 and target cells totally lysed by the addition of saponin at the end of the incubation serve as negative and positive controls, respectively.

Based on the measured remaining living target cells, the percentage of specific cell lysis is calculated according to the following formula: [1−(number of living targets(sample)/number of living targets(spontaneous))]×100%. Sigmoidal dose response curves and EC50 values are calculated by non-linear regression/4-parameter logistic fit using the GraphPad Software. The lysis values obtained for a given antibody concentration are used to calculate sigmoidal dose-response curves by 4 parameter logistic fit analysis using the Prism software.

### Example 36

### Synthesis of Ionizable Lipids

### 36.1 Synthesis of ((3-(2-methyl-1H-imidazol-1-yl)propyl)azanediyl)bis(hexane-6,1-diyl) bis(2-hexyldecanoate) (Lipid 27, Table 10a) and ((3-(1H-imidazol-1-yl)propyl)azanediyl)bis(hexane-6,1-diyl) bis(2-hexyldecanoate)) (Lipid 26, Table 10a)

In a 100 mL round bottom flask connected with condenser, 3-(1H-imidazol-1-yl)propan-1-amine (100 mg, 0.799 mmol) or 3-(2-methyl-1H-imidazol-1-yl)propan-1-amine (0.799 mmol), 6-bromohexyl 2-hexyldecanoate (737.2 mg, 1.757 mmol), potassium carbonate (485 mg, 3.515 mmol) and potassium iodide (13 mg, 0.08 mmol) were mixed in acetonitrile (30 mL), and the reaction mixture was heated to 80° C. for 48 h. The mixture was cooled to room temperature and was filtered through a pad of Celite. The filtrate was diluted with ethyl acetate. After washing with water, brine and dried over anhydrous sodium sulfate. The solvent was evaporated and the crude residue was purified by flash chromatography (SiO₂: CH₂Cl₂=100% to 10% of methanol in CH₂Cl₂) and colorless oil product was obtained (92 mg, 15%). Molecular formula of ((3-(1H-imidazol-1-yl)propyl)azanediyl)bis(hexane-6,1-diyl) bis(2-hexyldecanoate)) is C₅₀H₉₅N₃O₄ and molecular weight (M_{w}) is 801.7.

### Reaction Scheme for Synthesis of ((3-(1H-imidazol-1-yl)propyl)azanediyl)bis(hexane-6,1-diyl) bis(2-hexyldecanoate)) (Lipid 26, Table 10a)

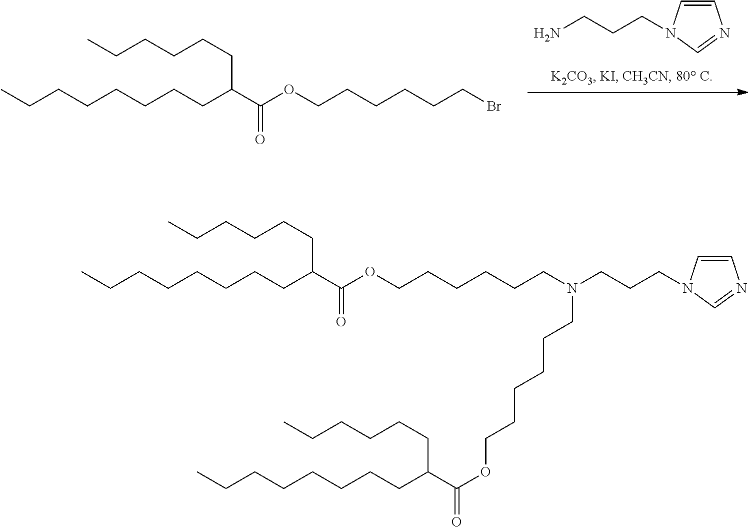 [SMILES: CCCCCCCCC(CCCCCC)C(=O)OCCCCCCBr ; NCCCn1ccnc1 ; CCCCCCCCC(CCCCCC)C(=O)OCCCCCCN(CCCCCCOC(=O)C(CCCCCC)CCCCCCCC)CCCn1ccnc1]

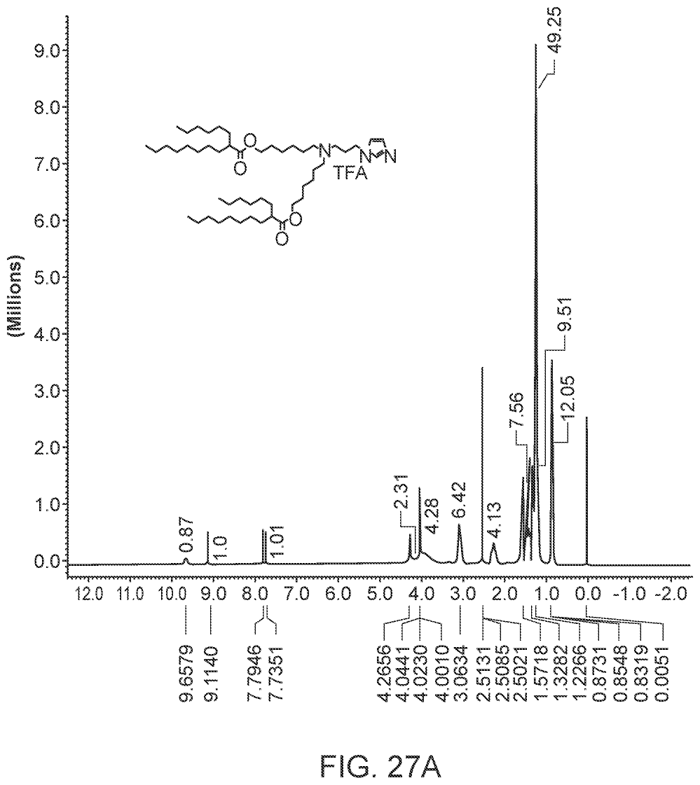

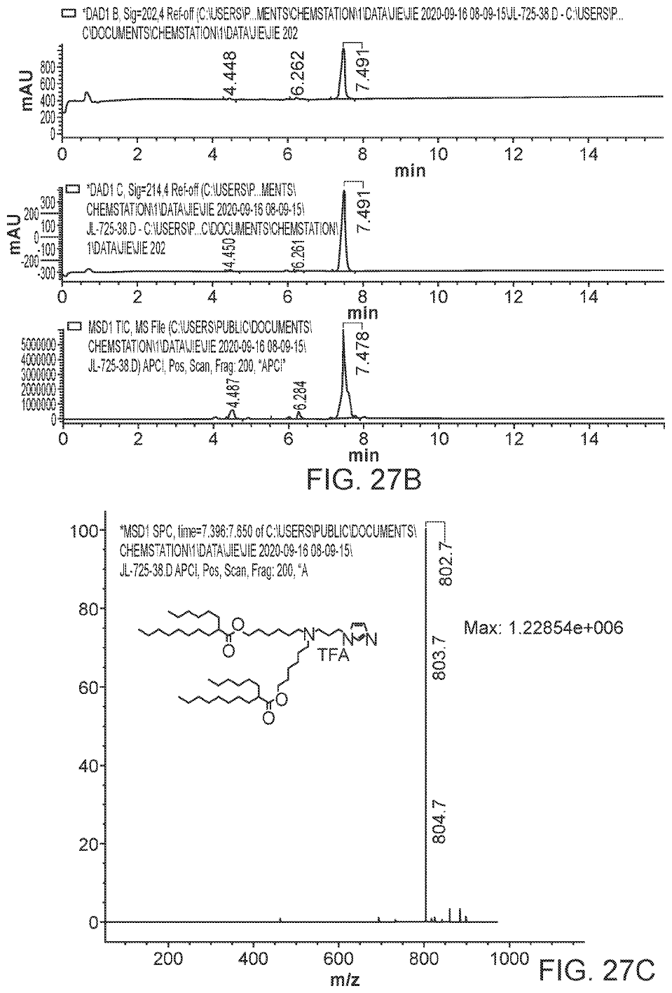

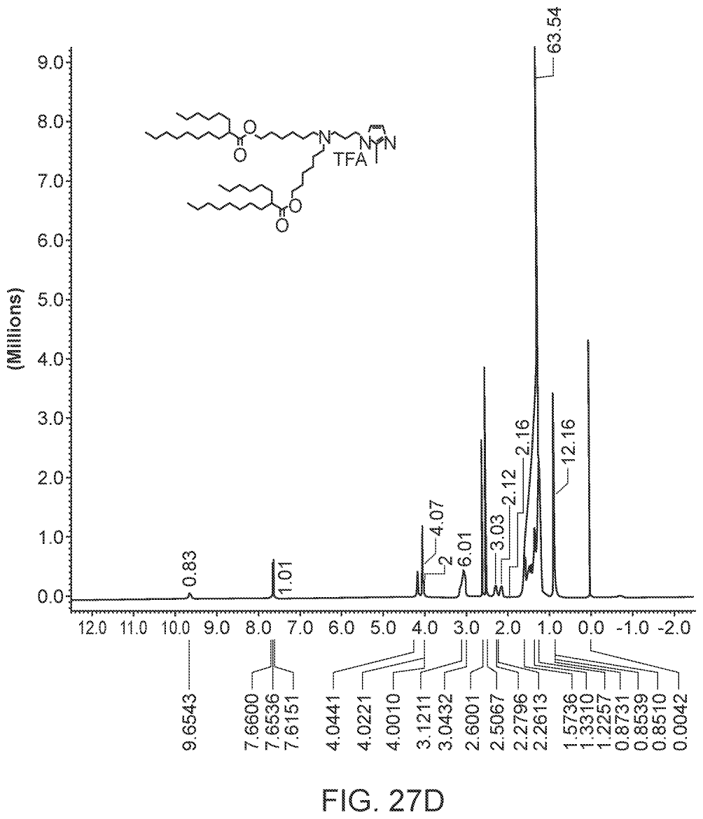

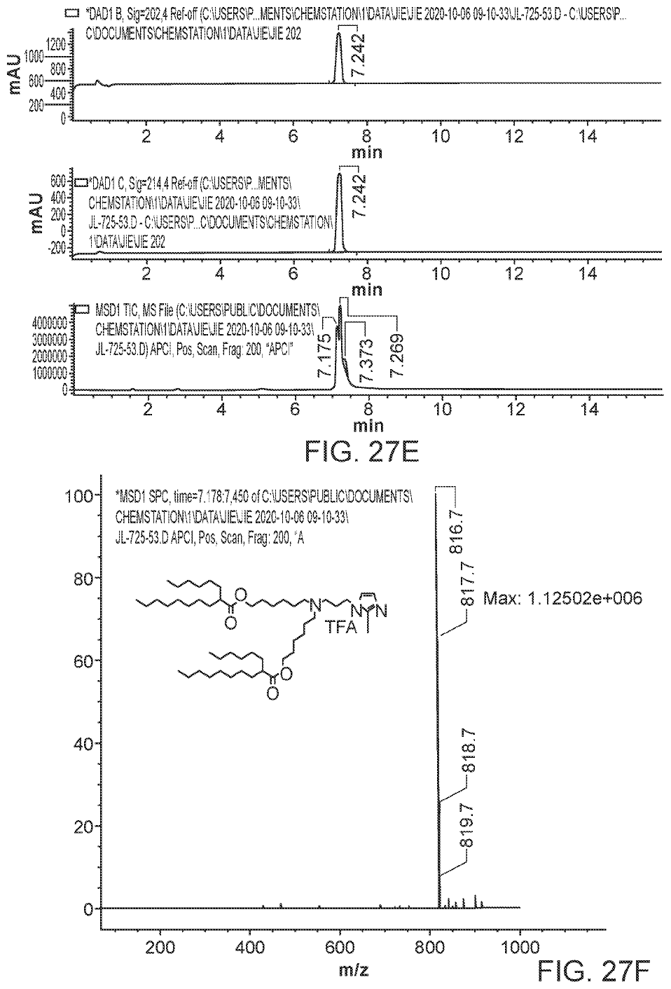

Characterization of Lipid 26 was performed by LC-MS. FIG. 27A-C shows characterization of Lipid 26. FIG. 27A shows the proton NMR observed for Lipid 26. FIG. 27B is a representative LC/MS trace for Lipid 26 with total ion and UV chromatograms shown.

### 36.2 Synthesis of Lipid 22-S14

### 36.2.1 Synthesis of 2-(tetradecylthio)ethan-1-ol

To a mixture of 2-sulfanylethanol (5.40 g, 69.11 mmol, 4.82 mL, 0.871 eq) in acetonitrile (200 mL) was added 1-Bromotetradecane(22 g, 79.34 mmol, 23.66 mL, 1 eq) and potassium carbonate (17.55 g, 126.95 mmol, 1.6 eq) at 25° C. The reaction mixture was warmed to 40° C. and stirred for 12 hr. TLC (ethyl acetate/petroleum ether=25/1, R_{f}=0.3, stained by I₂) showed the starting material was consumed completely and a new main spot was generated. The reaction mixture was filtered and the filter cake was washed with acetonitrile (50 mL) and then the filtrate was concentrated under vacuum to get a residue which was purified by column on silica gel (ethyl acetate/petroleum ether=1/100 to 1/25) to afford 2-(tetradecylthio)ethan-1-ol (14 g, yield 64.28%) as a white solid.

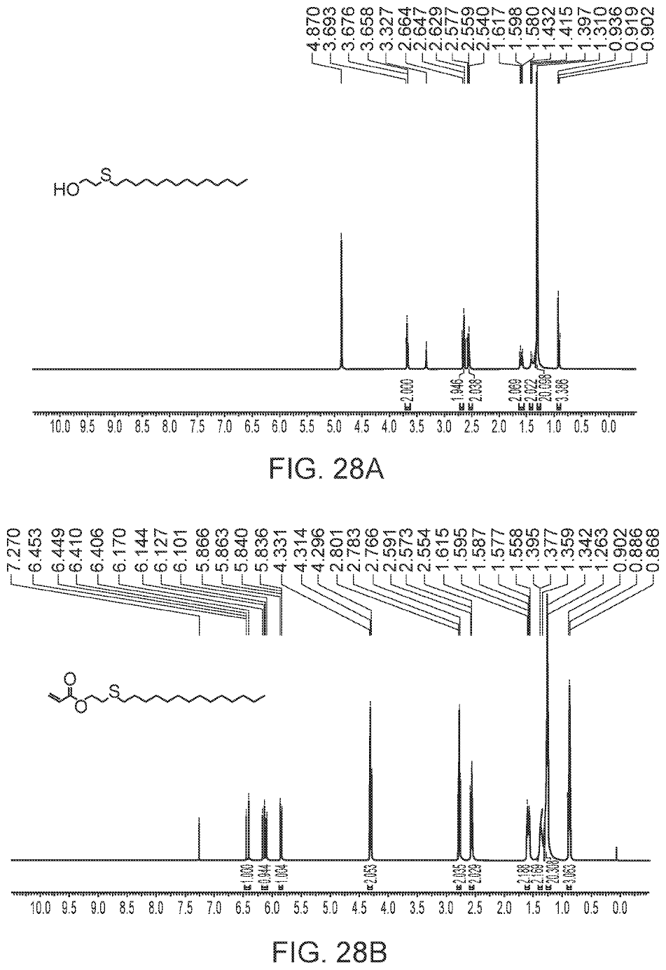

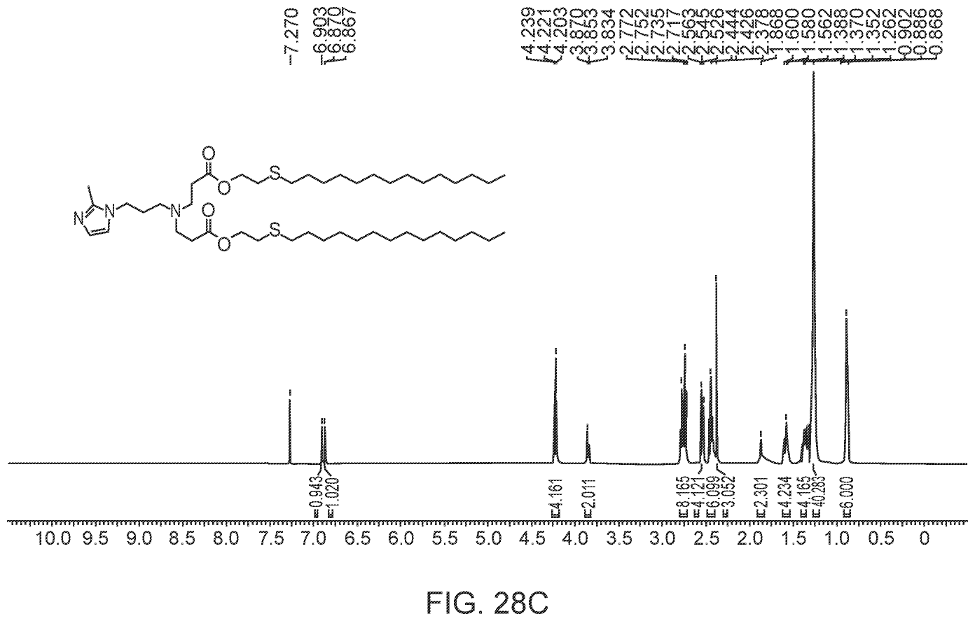

¹H NMR (ET36387-45-P1A, 400 MHz, CHLOROFORM-d) δ 0.87-0.91 (m, 3H) 1.27 (s, 20H) 1.35-1.43 (m, 2H) 1.53-1.64 (m, 2H) 2.16 (br s, 1H) 2.49-2.56 (m, 2H) 2.74 (t, J=5.93 Hz, 2H) 3.72 (br d, J=4.89 Hz, 2H). FIG. 28 shows corresponding Nuclear Magnetic Resonance (NMR) spectrum.

### 38.2.2 Synthesis of 2-(tetradecylthio)ethyl Acrylate

To a solution of 2-(tetradecylthio)ethan-1-ol (14 g, 51.00 mmol, 1 eq) in dichloromethane (240 mL) was added triethylamine (7.74 g, 76.50 mmol, 10.65 mL, 1.5 eq) and prop-2-enoyl chloride (5.54 g, 61.20 mmol, 4.99 mL, 1.2 eq) dropwise at 0° C. under nitrogen. The reaction mixture was warmed to 25° C. and stirred for 12 hr. TLC (ethyl acetate/petroleum ether=25/1, Rf=0.5, stained by I₂) showed the starting material was consumed completely and a new main spot was generated. The reaction solution was concentrated under vacuum to get crude which was purified by column on silica gel (ethyl acetate/petroleum ether=1/100 to 1/25) to afford 2-(tetradecylthio)ethyl acrylate (12 g, yield 71.61%) as a colorless oil.

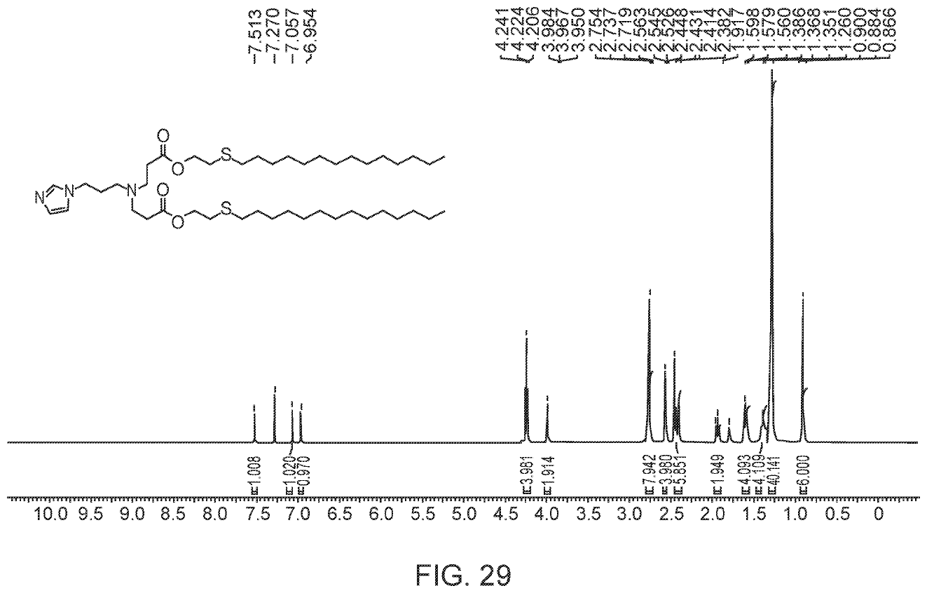

¹H NMR (ET36387-49-P1A, 400 MHz, CHLOROFORM-d) δ 0.85-0.93 (m, 3H) 1.26 (s, 19H) 1.35-1.43 (m, 2H) 1.53-1.65 (m, 2H) 2.53-2.62 (m, 2H) 2.79 (t, J=7.03 Hz, 2H) 4.32 (t, J=7.03 Hz, 2H) 5.86 (dd, J=10.39, 1.47 Hz, 1H) 6.09-6.19 (m, 1H) 6.43 (dd, J=17.30, 1.41 Hz, 1H). FIG. 29 shows corresponding Nuclear Magnetic Resonance (NMR) spectrum.

### 36.2.3 Synthesis of bis(2-(tetradecylthio)ethyl) 3,3′-((3-(2-methyl-1H-imidazol-1-yl)propyl)azanediyl)dipropionate (Lipid 22-S14)

A flask was charged with 3-(2-methyl-1H-imidazol-1-yl)propan-1-amine (300 mg, 2.16 mmol) and 2-(tetradecylthio)ethyl acrylate (1.70 g, 5.17 mmol). The neat reaction mixture was heated to 80° C. and stirred for 48 hr. TLC (ethyl acetate, R_{f}=0.3, stained by I₂, one drop ammonium hydroxide added) showed the starting material was consumed completely and a new main spot was formed. The reaction mixture was diluted with dichloromethane (4 mL) and purified by column on silica gel (petroleum ether/ethyl acetate=3/1 to 0/1, 0.1% ammonium hydroxide added) to get bis(2-(tetradecylthio)ethyl) 3,3′-((3-(2-methyl-1H-imidazol-1-yl)propyl)azanediyl)dipropionate (501 mg, yield 29.1%) as colorless oil.

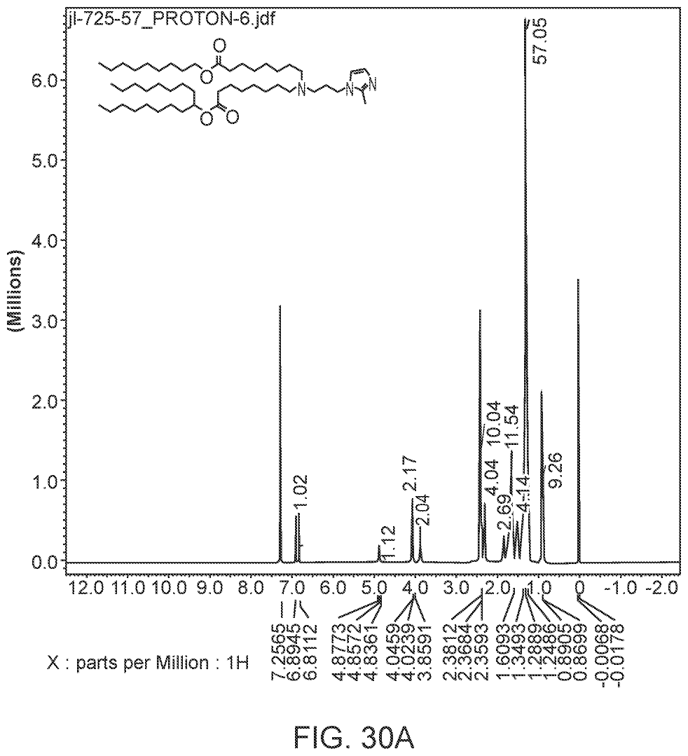

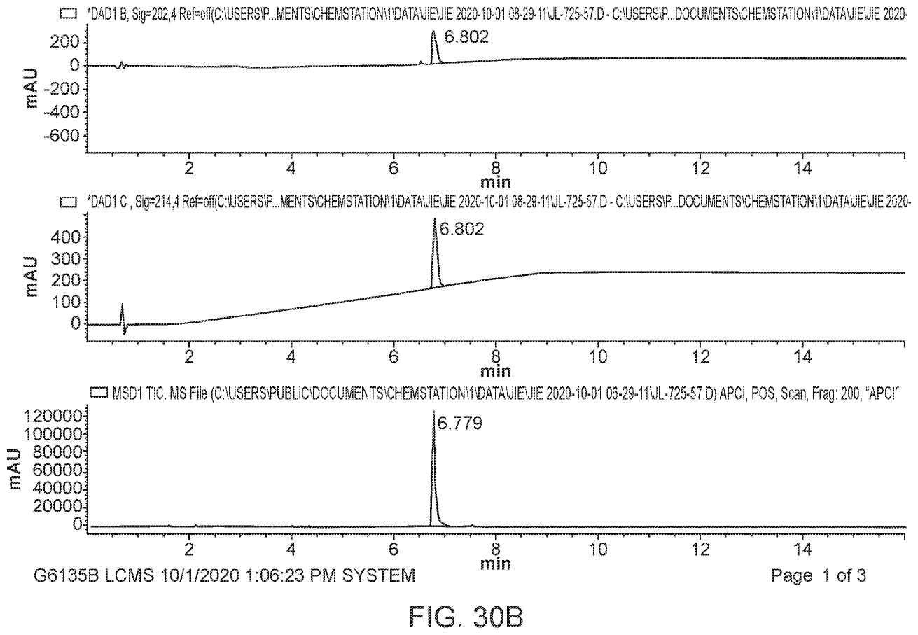

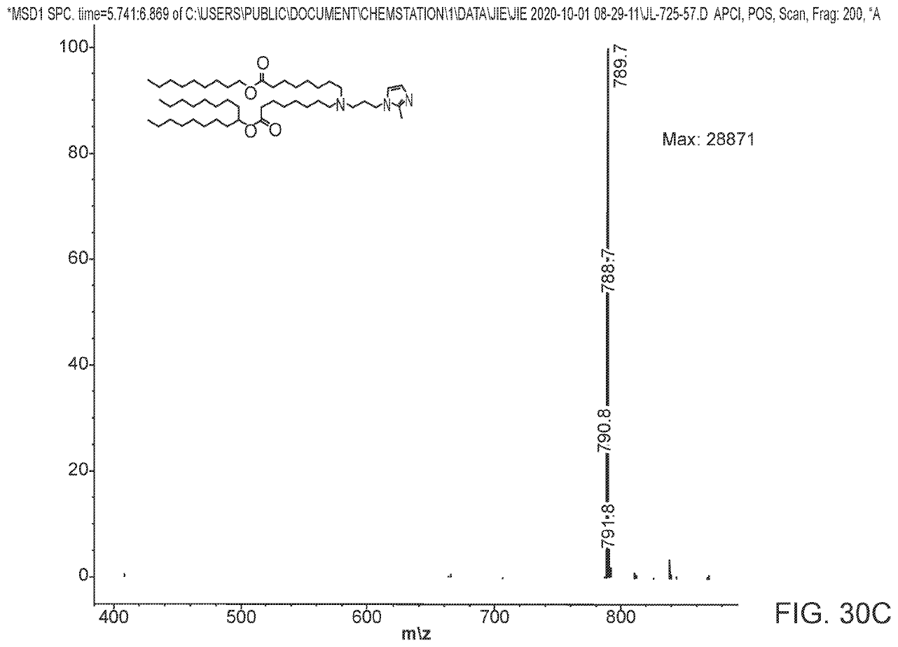

¹H NMR (ET36387-51-P1A, 400 MHz, CHLOROFORM-d) δ 0.87 (t, J=6.73 Hz, 6H) 1.25 (s, 40H) 1.33-1.40 (m, 4H) 1.52-1.61 (m, 4H) 1.81-1.90 (m, 2H) 2.36 (s, 3H) 2.39-2.46 (m, 6H) 2.53 (t, J=7.39 Hz, 4H) 2.70-2.78 (m, 8H) 3.84 (t, J=7.17 Hz, 2H) 4.21 (t, J=6.95 Hz, 4H) 6.85 (s, 1H) 6.89 (s, 1H). FIG. 30 shows corresponding Nuclear Magnetic Resonance (NMR) spectrum.

### 36.3 Synthesis of bis(2-(tetradecylthio)ethyl) 3,3′-((3-(1H-imidazol yl)propyl)azanediyl)dipropionate (Lipid 93-S14)

A flask was charged with 3-(1H-imidazol-1-yl)propan-1-amine (300 mg, 2.40 mmol, 1 eq) and 2-(tetradecylthio)ethyl acrylate (1.89 g, 5.75 mmol, 2.4 eq). The neat reaction mixture was heated to 80° C. and stirred for 48 hr. TLC (ethyl acetate, R_{f}=0.3, stained by I₂, one drop ammonium hydroxide added) showed the starting material was consumed completely and a new main spot was formed. The reaction mixture was diluted with dichloromethane (4 mL) and purified by column on silica gel (petroleum ether/ethyl acetate=1/20-0/100, 0.1% ammonium hydroxide added) to get bis(2-(tetradecylthio)ethyl) 3,3′-((3-(1H-imidazol-1-yl)propyl)azanediyl)dipropionate (512 mg, yield 27.22%) as colorless oil.

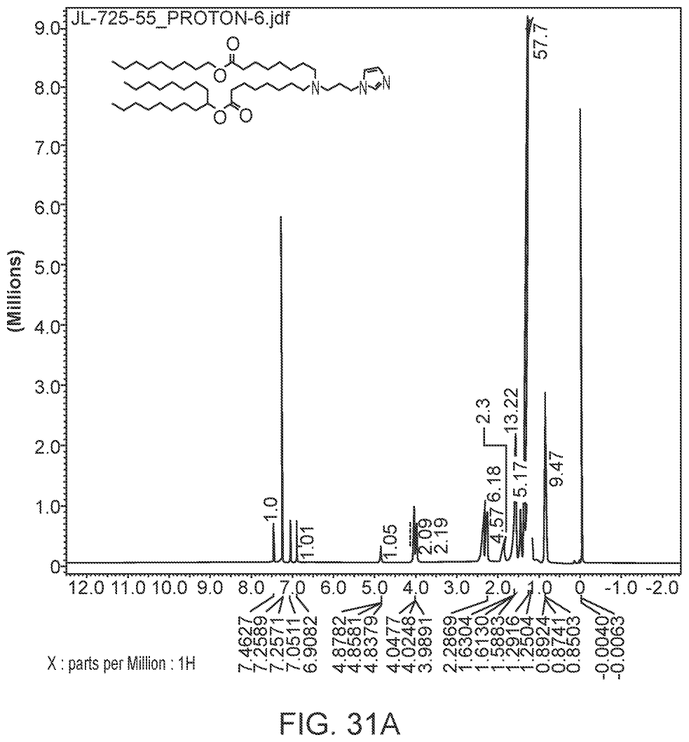

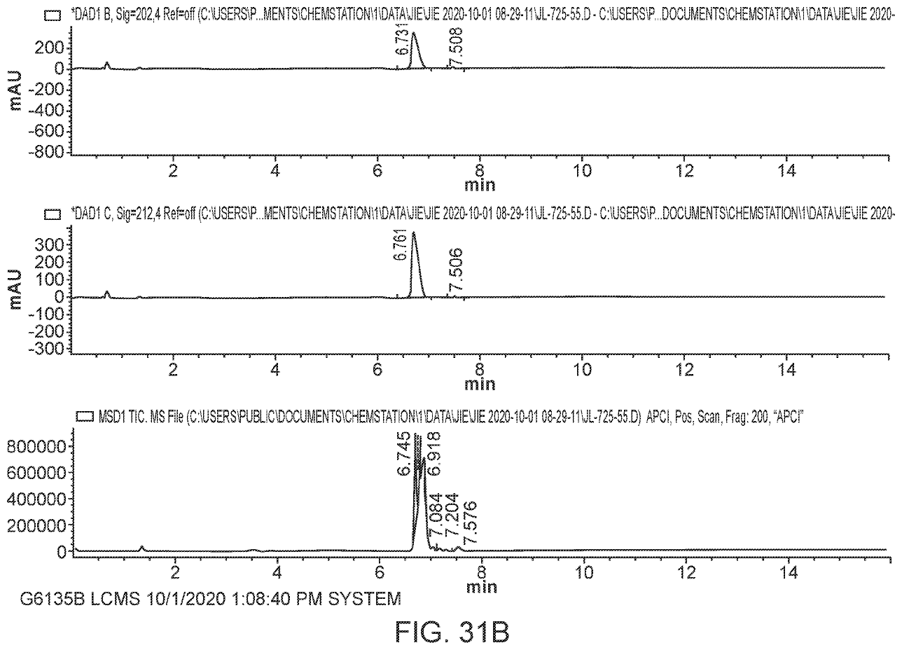

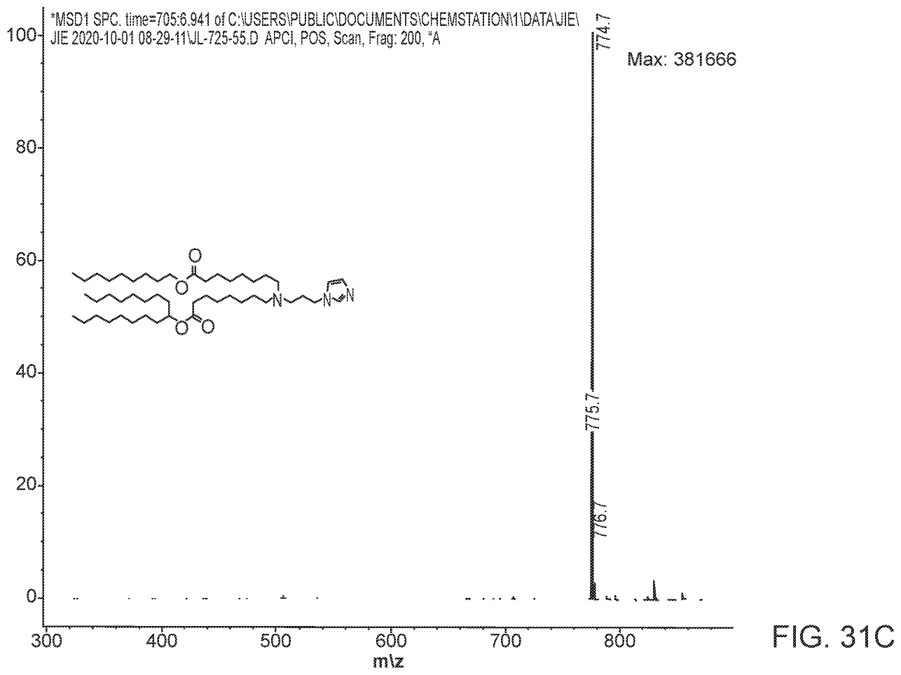

¹H NMR (ET36387-54-P1A, 400 MHz, CHLOROFORM-d) δ 0.89 (t, J=6.84 Hz, 6H) 1.26 (s, 40H) 1.34-1.41 (m, 4H) 1.58 (br t, J=7.50 Hz, 4H) 1.92 (t, J=6.62 Hz, 2H) 2.36-2.46 (m, 6H) 2.55 (t, J=7.50 Hz, 4H) 2.75 (q, J=6.84 Hz, 8H) 3.97 (t, J=6.95 Hz, 2H) 4.23 (t, J=6.95 Hz, 4H) 6.95 (s, 1H) 7.06 (s, 1H) 7.51 (s, 1H). FIG. 31 shows corresponding Nuclear Magnetic Resonance (NMR) spectrum.

### 36.4 Synthesis of heptadecan-9-yl 8-((3-(2-methyl-1H-imidazol-1-yl)propyl)(8-(nonyloxy)-8-oxooctyl)amino)octanoate (Lipid 54, Table 10a)

### 36.4.1 Synthesis of nonyl 8-bromooctanoate (3)

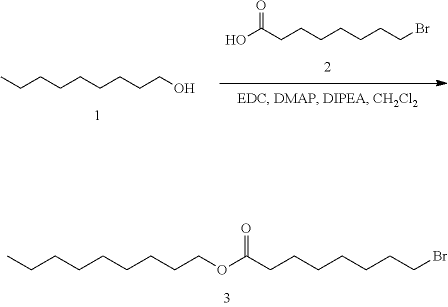

To a mixture of 8-bromooctanoic acid (2) (18.6 g, 83.18 mmol) and nonan-1-ol (1) (10 g, 69.32 mmol) in CH₂Cl₂ (500 mL) was added DMAP (1.7 g, 13.86 mmol), DIPEA (48 mL, 277.3 mmol) and EDC (16 g, 83.18 mmol). The reaction was stirred at room temperature overnight. After concentration of the reaction mixture, the crude residue was dissolved in ethyl acetate (500 mL), washed with 1N HCl, sat. NaHCO₃, water and Brine. The organic layer was dried over anhydrous Na₂SO₄. The solvent was evaporated and the crude residue was purified by flash chromatography (SiO₂: Hexane=100% to 30% of EtOAc in Hexane) and colorless oil product 3 was obtained (9 g, 37%).

### 36.4.2 Synthesis of heptadecan-9-yl 8-bromooctanoate (5)

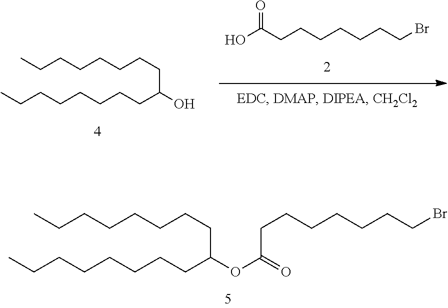 [SMILES: O=C(O)CCCCCCCBr ; CCCCCCCCC(O)CCCCCCCC ; CCCCCCCCC(CCCCCCCC)OC(=O)CCCCCCCBr]

To a mixture of 8-bromooctanoic acid (2) (10 g, 44.82 mmol) and heptadecan-9-ol (4) (9.6 g, 37.35 mmol) in CH₂Cl₂ (300 mL) was added DMAP (900 mg, 7.48 mmol), DIPEA (26 mL, 149.7 mmol) and EDC (10.7 g, 56.03 mmol). The reaction was stirred at room temperature overnight. After concentration of the reaction mixture, the crude residue was dissolved in ethyl acetate (300 mL), washed with 1N HCl, sat. NaHCO₃, water and Brine. The organic layer was dried over anhydrous Na₂SO₄. The solvent was evaporated and the crude residue was purified by flash chromatography (SiO₂: Hexane=100% to 30% of EtOAc in Hexane) and colorless oil product 5 was obtained (5 g, 29%).

¹H NMR (300 MHz, CDCl₃): δ ppm 4.86 (m, 1H), 3.39 (t, J=7.0 Hz, 2H), 2.27 (t, J=7.6 Hz, 2H), 1.84 (m, 2H), 1.62 (m, 2H), 1.5-1.4 (m, 8H), 1.35-1.2 (m, 26H) 0.87 (t, J=6.7 Hz, 6H).

### 36.4.3 Synthesis of heptadecan-9-yl 8-((3-(2-methyl-1H-imidazol-1-yl)propyl)amino)octanoate (7)

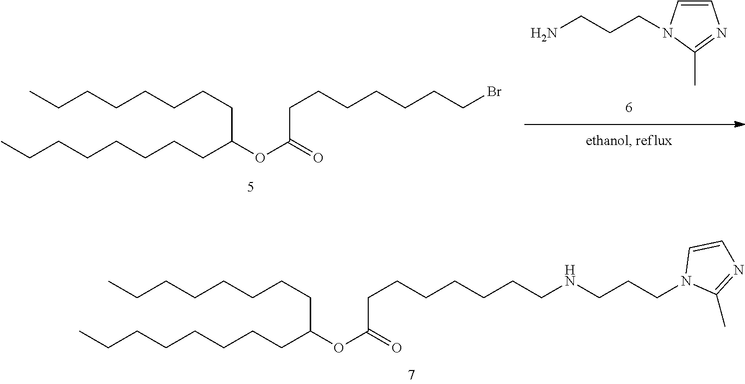

In a 100 mL round bottom flask connected with condenser, heptadecan-9-yl 8-bromooctanoate (5) (860 mg, 1.868 mmol) and 3-(2-methyl-1H-imidazol-1-yl)propan-1-amine (6) (1.3 g, 9.339 mmol) were mixed in ethanol (10 mL). The reaction mixture was heated to reflux overnight. MS (APCI) showed the expected product. The mixture was cooled to room temperature and concentrated. The crude residue was purified by flash chromatography (SiO₂:CH₂Cl₂=100% to 10% of methanol+1% NH₄OH in CH₂Cl₂) and colorless oil product 7 was obtained (665 mg, 69%).

### 36.4.4 Synthesis of heptadecan-9-yl 8-((3-(2-methyl-1H-imidazol-1-yl)propyl)(8-(nonyloxy)-8-oxooctyl)amino)octanoate (Lipid 54, Table 10a)

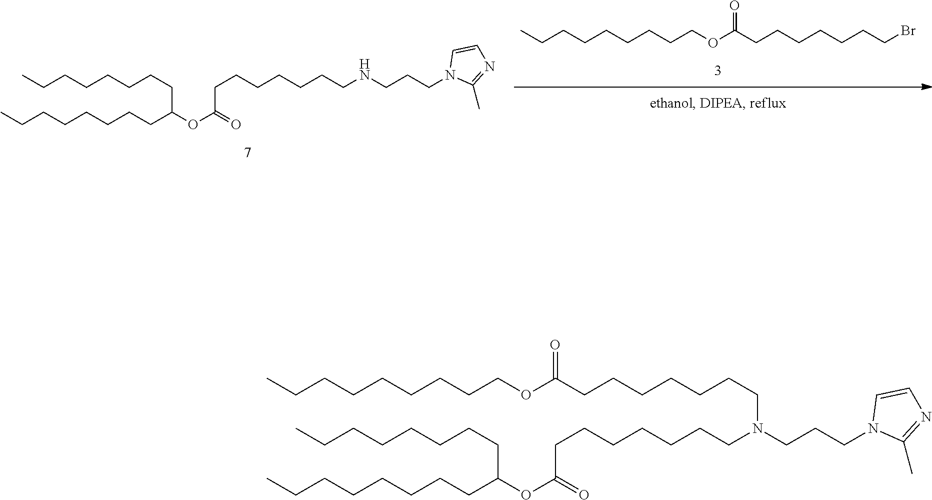 [SMILES: CCCCCCCCCOC(=O)CCCCCCCBr ; CCCCCCCCC(CCCCCCCC)OC(=O)CCCCCCCNCCCn1ccnc1C ; CCCCCCCCCOC(=O)CCCCCCCN(CCCCCCCC(=O)OC(CCCCCCCC)CCCCCCCC)CCCn1ccnc1C]

In a 100 mL round bottom flask connected with condenser, heptadecan-9-yl 8-((3-(2-methyl-1H-imidazol-1-yl)propyl)amino)octanoate (7) (665 mg, 1.279 mmol) and nonyl 8-bromooctanoate (3) (536 mg, 1.535 mmol) were mixed in ethanol (10 mL), then DIPEA (0.55 mL, 3.198 mmol) was added. The reaction mixture was heated to reflux overnight. Both MS (APCI) and TLC (10% MeOH+1% NH₄OH in CH₂Cl₂) showed the product and some unreacted starting material. The mixture was cooled to room temperature and concentrated. The crude residue was purified by flash chromatography (SiO₂: CH₂Cl₂=100% to 10% of methanol+1% NH₄OH in CH₂Cl₂) and colorless oil was obtained (170 mg, 17%).

### 36.5 Synthesis of heptadecan-9-yl 8-((3-(1H-imidazol-1-yl)propyl)(8-(nonyloxy)-8-oxooctyl)amino)octanoate (Lipid 53, Table 10a)

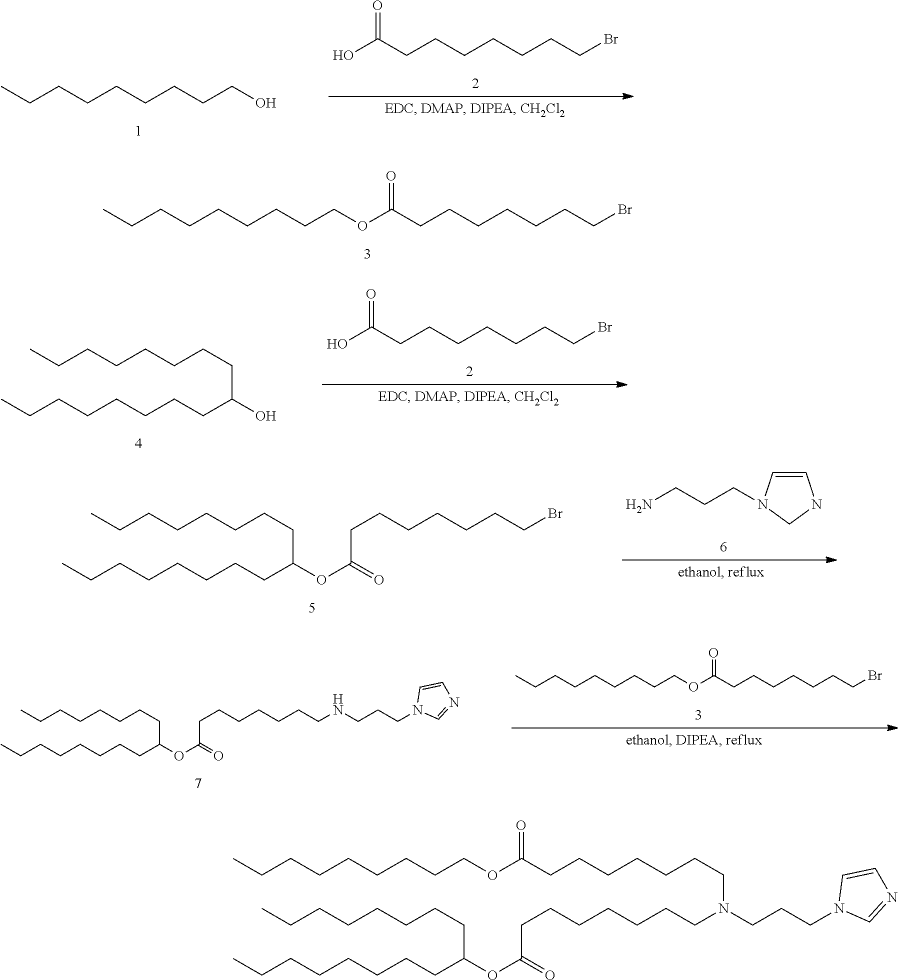

Lipid 53 from Table 10a is synthesized according to the scheme above. Reaction conditions are identical to Lipid 54 with the exception of 3-(1H-imidazol-1-yl)propan-1-amine as the imidazole amine.

### 36.6 Synthesis of Heptadecan-9-yl 8-((3-(1H-imidazol-1-yl)propyl)(8-(nonyloxy)-8-oxooctyl)amino)octanoate (Lipid 45, Table 10a)

### 36.6.1 Synthesis of heptadecan-9-yl 8-bromooctanoate (3)

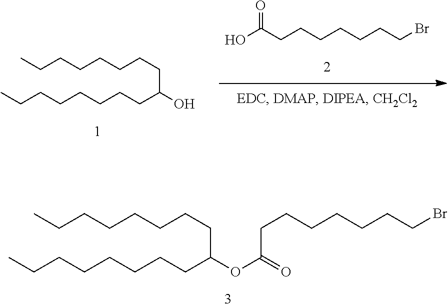 [SMILES: O=C(O)CCCCCCCBr ; CCCCCCCCC(O)CCCCCCCC ; CCCCCCCCC(CCCCCCCC)OC(=O)CCCCCCCBr]

To a mixture of 8-bromooctanoic acid (2) (10 g, 44.82 mmol) and heptadecan-9-ol (1) (9.6 g, 37.35 mmol) in CH₂Cl₂ (300 mL) was added DMAP (900 mg, 7.48 mmol), DIPEA (26 mL, 149.7 mmol) and EDC (10.7 g, 56.03 mmol). The reaction was stirred at room temperature overnight. After concentration of the reaction mixture, the crude residue was dissolved in ethyl acetate (300 mL), washed with 1N HCl, sat. NaHCO₃, water and Brine. The organic layer was dried over anhydrous Na₂SO₄. The solvent was evaporated and the crude residue was purified by flash chromatography (SiO₂: Hexane=100% to 30% of EtOAc in Hexane) and colorless oil product 3 was obtained (5 g, 29%).

¹H NMR (300 MHz, CDCl₃): δ ppm 4.86 (m, 1H), 3.39 (t, J=7.0 Hz, 2H), 2.27 (t, J=7.6 Hz, 2H), 1.84 (m, 2H), 1.62 (m, 2H), 1.5-1.4 (m, 8H), 1.35-1.2 (m, 26H) 0.87 (t, J=6.7 Hz, 6H).

### 36.6.2 Synthesis of heptadecan-9-yl 8-((3-(1H-imidazol-1-yl)propyl)amino)octanoate (6)

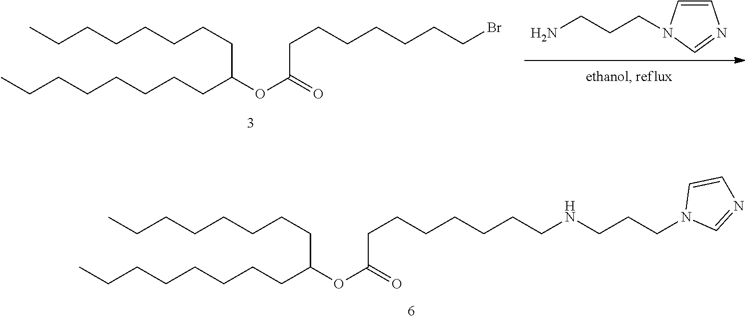 [SMILES: CCCCCCCCC(CCCCCCCC)OC(=O)CCCCCCCBr ; NCCCn1ccnc1 ; CCCCCCCCC(CCCCCCCC)OC(=O)CCCCCCCNCCCn1ccnc1]

In a 100 mL round bottom flask connected with condenser, heptadecan-9-yl 8-bromooctanoate (3) (1 g, 2.167 mmol) and 3-(1H-imidazol-1-yl)propan-1-amine (4) (1.3 mL, 10.83 mmol) were mixed in ethanol (10 mL). The reaction mixture was heated to reflux overnight. MS (APCI) showed the expected product. The mixture was cooled to room temperature and concentrated. The crude residue was purified by flash chromatography (SiO₂: CH₂Cl₂=100% to 10% of methanol+1% NH₄OH in CH₂Cl₂) and colorless oil product 6 was obtained (498 mg, 45%).

¹H NMR (300 MHz, CDCl₃): δ ppm 7.47 (s, 1H), 7.04 (s, 1H), 6.91 (s, 1H), 4.85 (m, 1H), 4.03 (t, J=7.0 Hz, 2H), 2.56 (dd, J=14.5, 7.4 Hz, 4H), 2.27 (t, J=7.4 Hz, 2H), 1.92 (m, 2H), 1.60 (m, 2H), 1.48 (m, 6H), 1.30-1.20 (m, 31H), 0.86 (t, J=6.6 Hz, 6H). MS (APCI⁺): 506.4 (M+1).

### 36.6.3 Synthesis of nonyl 8-bromooctanoate (9)

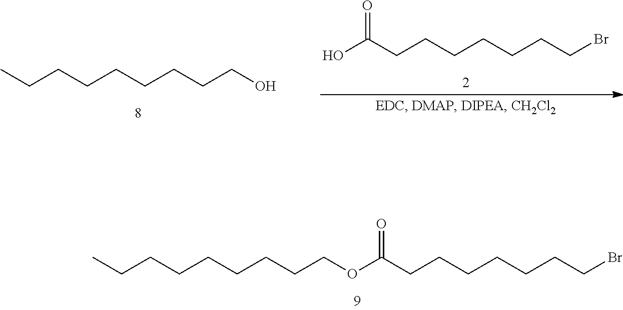

To a mixture of 8-bromooctanoic acid (2) (18.6 g, 83.18 mmol) and nonan-1-ol (8) (10 g, 69.32 mmol) in CH₂Cl₂ (500 mL) was added DMAP (1.7 g, 13.86 mmol), DIPEA (48 mL, 277.3 mmol) and EDC (16 g, 83.18 mmol). The reaction was stirred at room temperature overnight. After concentration of the reaction mixture, the crude residue was dissolved in ethyl acetate (500 mL), washed with 1N HCl, sat. NaHCO₃, water and Brine. The organic layer was dried over anhydrous Na₂SO₄. The solvent was evaporated and the crude residue was purified by flash chromatography (SiO₂: Hexane=100% to 30% of EtOAc in Hexane) and colorless oil product 9 was obtained (9 g, 37%).

¹H NMR (300 MHz, CDCl₃): δ ppm 4.05 (t, J=7.0 Hz, 2H), 3.39 (t, J=7.0 Hz, 2H), 2.29 (t, J=7.6 Hz, 2H), 1.84 (m, 2H), 1.62-1.56 (m, 6H), 1.40-1.20 (m, 16H), 0.87 (t, J=6.7 Hz, 3H).

### 36.6.4 Synthesis of heptadecan-9-yl 8-((3-(1H-imidazol-1-yl)propyl)(8-(nonyloxy) oxooctyl)amino)octanoate

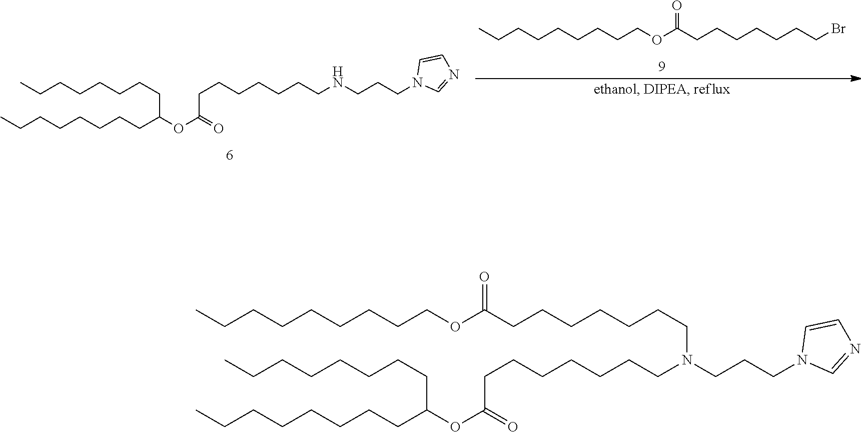 [SMILES: CCCCCCCCCOC(=O)CCCCCCCBr ; CCCCCCCCC(CCCCCCCC)OC(=O)CCCCCCCNCCCn1ccnc1 ; CCCCCCCCCOC(=O)CCCCCCCN(CCCCCCCC(=O)OC(CCCCCCCC)CCCCCCCC)CCCn1ccnc1]

In a 100 mL round bottom flask connected with condenser, heptadecan-9-yl 8-((3-(1H-imidazol-1-yl)propyl)amino)octanoate (6) (242 mg, 0.478 mmol) and nonyl 8-bromooctanoate 9 (200 mg, 0.574 mmol) were mixed in ethanol (10 mL), then DIPEA (0.2 mL, 1.196 mmol) was added. The reaction mixture was heated to reflux overnight. Both MS (APCI) and TLC (10% MeOH+1% NH₄OH in CH₂Cl₂) showed the product and some unreacted starting material. The mixture was cooled to room temperature and concentrated. The crude residue was purified by flash chromatography (SiO₂: CH₂Cl₂=100% to 10% of methanol+1% NH₄OH in CH₂Cl₂) and colorless oil was obtained (35 mg, 10%).

¹H NMR (300 MHz, CDCl3): δ ppm 7.46 (s, 1H), 7.05 (s, 1H), 6.90 (s, 1H), 4.85 (m, 1H), 4.04 (t, J=6.6 Hz, 2H), 4.01 (t, J=6.6 Hz, 2H), 2.38 (m, 6H), 2.27 (t, J=3.8 Hz, 4H), 1.89 (m, 2H), 1.60-1.58 (m, 12H), 1.48 (m, 6H), 1.30-1.20 (m, 47H), 0.87 (t, J=7.1 Hz, 9H). MS (APCI+): 774.6 (M+1).

### 36.7 Synthesis of Heptadecan-9-yl 8-((3-(2-methyl-1H-imidazol-1-yl)propyl)(8-(nonyloxy)-8-oxooctyl)amino)octanoate (Lipid 46, Table 10a)

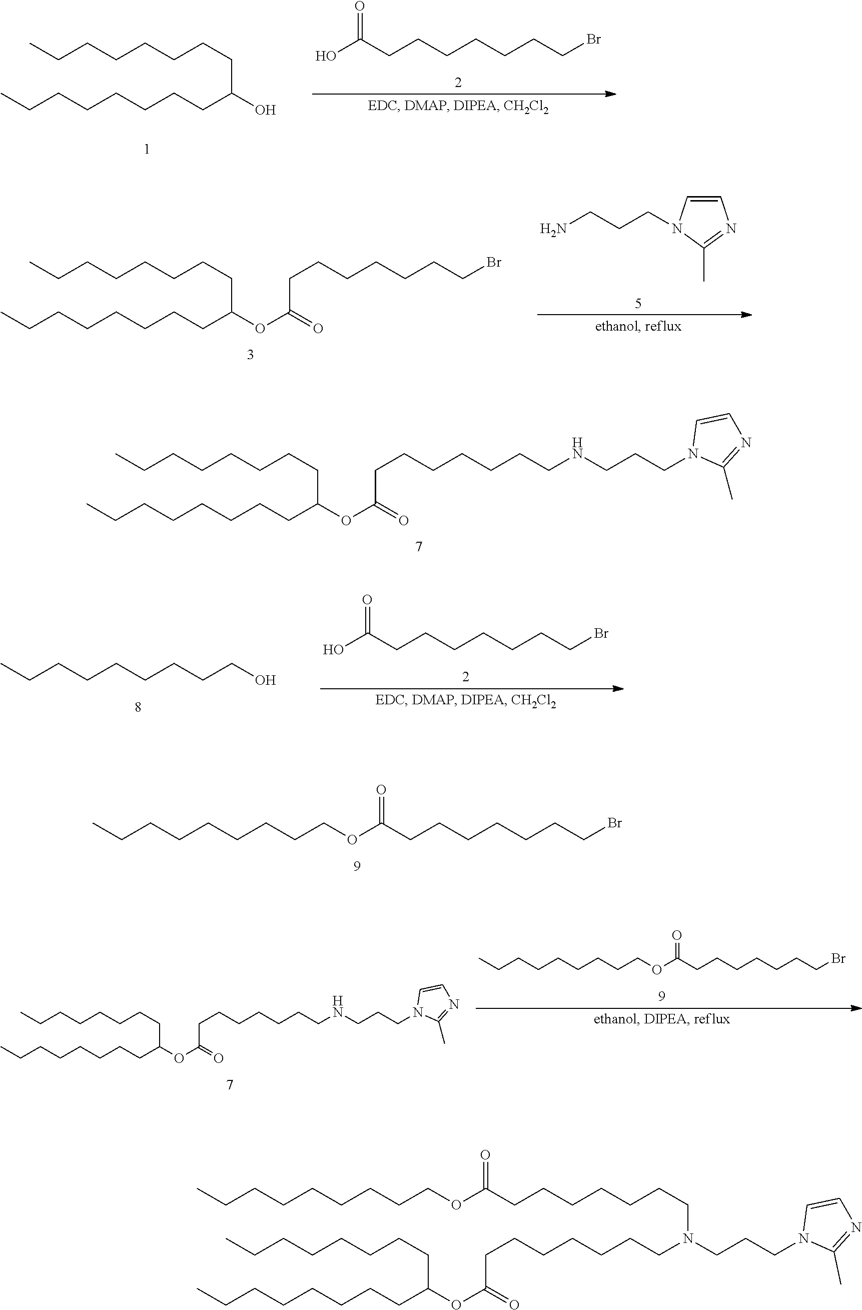

Lipid 46 from Table 10a is synthesized according to the scheme above. Reaction conditions are identical to Lipid 45 with the exception of 3-(2-Methyl-1H-imidazol-1-yl)propan-1-amine as the imidazole amine.

¹H NMR (300 MHz, CDCl₃): δ ppm 6.89 (s, 1H), 6.81 (s, 1H), 4.86 (m, 1H), 4.04 (t, J=6.8 Hz, 2H), 3.85 (t, J=7.4 Hz, 2H), 2.38-2.36 (m, 9H), 2.28 (m, 4H), 1.82 (m, 2H), 1.72-1.56 (m, 12H), 1.48 (m, 4H), 1.30-1.20 (m, 46H), 0.86 (t, J=6.6 Hz, 9H). MS (APCI⁺): 789.7 (M+1).

### 36.8 Synthesis of Heptadecan-9-yl 8-((3-(1H-imidazol-1-yl)propyl)(8-oxo-8-(undecan yloxy)octyl)amino)octanoate (Lipid 137, Table 10a)

### 36.8.1 Synthesis of heptadecan-9-yl 8-bromooctanoate (3)

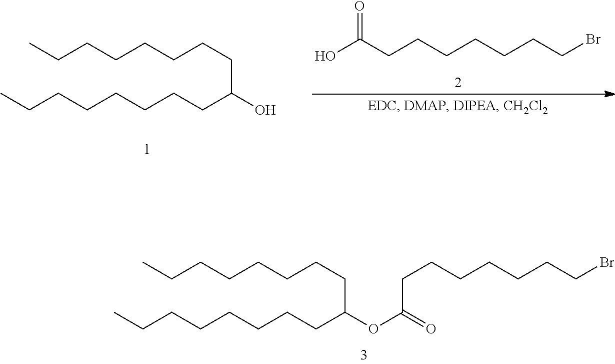 [SMILES: CCCCCCCCC(O)CCCCCCCC ; O=C(O)CCCCCCCBr ; CCCCCCCCC(CCCCCCCC)OC(=O)CCCCCCCBr]

To a mixture of 8-bromooctanoic acid (2) (10 g, 44.82 mmol) and heptadecan-9-ol (1) (9.6 g, 37.35 mmol) in CH₂Cl₂ (300 mL) was added DMAP (900 mg, 7.48 mmol), DIPEA (26 mL, 149.7 mmol) and EDC (10.7 g, 56.03 mmol). The reaction was stirred at room temperature overnight. After concentration of the reaction mixture, the crude residue was dissolved in ethyl acetate (300 mL), washed with 1N HCl, sat. NaHCO₃, water and Brine. The organic layer was dried over anhydrous Na₂SO₄. The solvent was evaporated and the crude residue was purified by flash chromatography (SiO₂: Hexane=100% to 30% of EtOAc in Hexane) and colorless oil product 3 was obtained (5 g, 29%).

1H NMR (300 MHz, CDCl3): δ ppm 4.86 (m, 1H), 3.39 (t, J=7.0 Hz, 2H), 2.27 (t, J=7.6 Hz, 2H), 1.84 (m, 2H), 1.62 (m, 2H), 1.5-1.4 (m, 8H), 1.35-1.2 (m, 26H) 0.87 (t, J=6.7 Hz, 6H).

### 36.8.2 Synthesis of heptadecan-9-yl 8-((3-(1H-imidazol-1-yl)propyl)amino)octanoate (6)

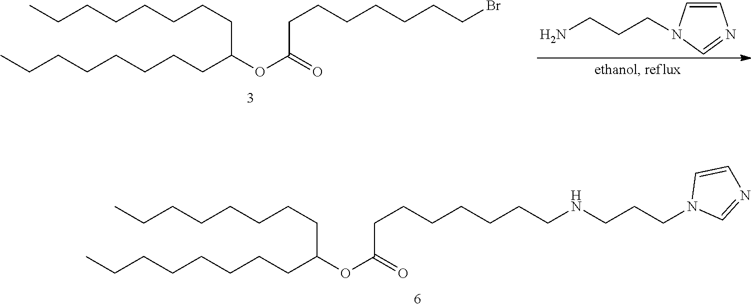 [SMILES: CCCCCCCCC(CCCCCCCC)OC(=O)CCCCCCCBr ; NCCCn1ccnc1 ; CCCCCCCCC(CCCCCCCC)OC(=O)CCCCCCCNCCCn1ccnc1]

In a 100 mL round bottom flask connected with condenser, heptadecan-9-yl 8-bromooctanoate (3) (1 g, 2.167 mmol) and 3-(1H-imidazol-1-yl)propan-1-amine (4) (1.3 mL, 10.83 mmol) were mixed in ethanol (10 mL). The reaction mixture was heated to reflux overnight. MS (APCI) showed the expected product. The mixture was cooled to room temperature and concentrated. The crude residue was purified by flash chromatography (SiO₂: CH₂Cl₂=100% to 10% of methanol+1% NH₄OH in CH₂Cl₂) and colorless oil product 6 was obtained (498 mg, 45%).

¹H NMR (300 MHz, CDCl₃): δ ppm 7.47 (s, 1H), 7.04 (s, 1H), 6.91 (s, 1H), 4.85 (m, 1H), 4.03 (t, J=7.0 Hz, 2H), 2.56 (dd, J=14.5, 7.4 Hz, 4H), 2.27 (t, J=7.4 Hz, 2H), 1.92 (m, 2H), 1.60 (m, 2H), 1.48 (m, 6H), 1.30-1.20 (m, 31H), 0.86 (t, J=6.6 Hz, 6H). MS (APCI⁺): 506.4 (M+1).

### 36.8.3 Synthesis of undecan-3-ol (11)

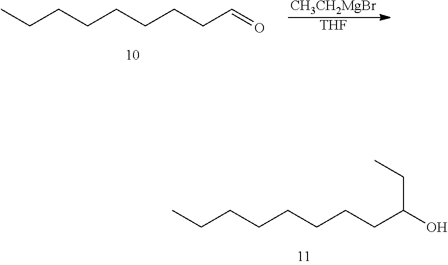

To a mixture of nonanal (10) (5 g, 35.2 mmol), in anhydrous THF (100 mL) at 0° C. ice-water bath was dropwise added ethylmagnesium bromide (47 mL, 42.2 mmol, 0.9M in THF). The reaction was stirred at room temperature overnight. The reaction was quenched with ice and diluted with ethyl acetate (500 mL), washed with 1N HCl, sat. NaHCO₃, water and Brine. The organic layer was dried over anhydrous Na₂SO₄. The solvent was evaporated and the crude residue was purified by flash chromatography (SiO₂: Hexane=100% to 50% of EtOAc in Hexane) and colorless oil product 11 was obtained (4 g, 66%).

¹H NMR (300 MHz, CDCl₃): δ ppm 3.52 (m, 1H), 1.56-1.3 (m, 4H), 1.3-1.20 (m, 12H), 0.93 (t, J=7.4 Hz, 3H), 0.87 (t, J=7.4 Hz, 3H).

### 36.8.4 Synthesis of undecan-3-yl 8-bromooctanoate (12)

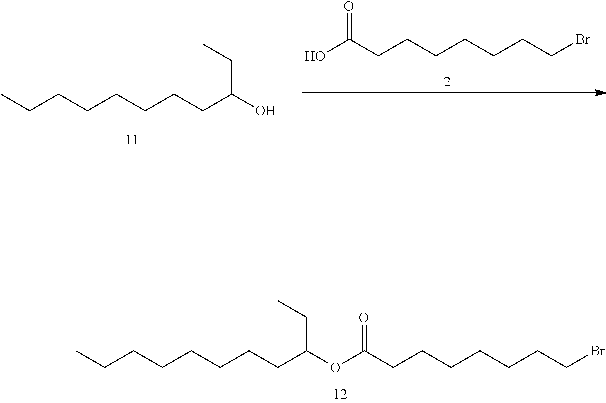 [SMILES: CCCCCCCCC(O)CC ; O=C(O)CCCCCCCBr ; CCCCCCCCC(CC)OC(=O)CCCCCCCBr]

To a mixture of 8-bromooctanoic acid (2) (6.2 g, 27.9 mmol) and undecan-3-ol (11) (4 g, 23.2 mmol) in CH₂Cl₂ (100 mL) was added DMAP (567.2 mg, 4.64 mmol), DIPEA (16.2 mL, 92.9 mmol) and EDC (6.7 g, 34.8 mmol). The reaction was stirred at room temperature overnight. After concentration of the reaction mixture, the crude residue was dissolved in ethyl acetate (500 mL), washed with 1N HCl, sat. NaHCO₃, water and Brine. The organic layer was dried over anhydrous Na₂SO₄. The solvent was evaporated and the crude residue was purified by flash chromatography (SiO₂: Hexane=100% to 30% of EtOAc in Hexane) and colorless oil product 12 was obtained (7.3 g, 83%).

¹H NMR (300 MHz, CDCl₃): δ ppm 4.80 (m, 1H), 3.39 (t, J=6.8 Hz, 2H), 2.28 (t, J=7.7 Hz, 2H), 1.84 (m, 2H), 1.6-1.35 (m, 8H), 1.35-1.2 (m, 16H), 0.87 (t, J=7.4 Hz, 6H).

### 36.8.4 Synthesis of heptadecan-9-yl 8-((3-(1H-imidazol-1-yl)propyl)(8-(nonyloxy) oxooctyl)amino)octanoate

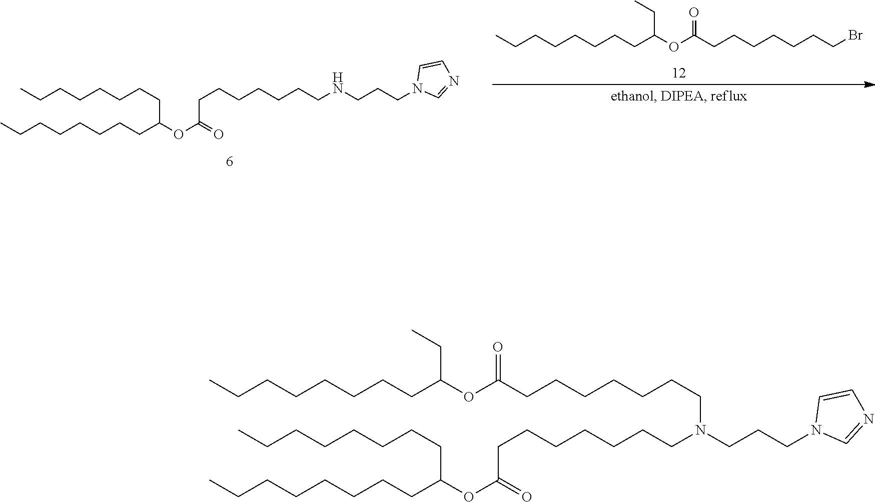 [SMILES: CCCCCCCCC(CC)OC(=O)CCCCCCCBr ; CCCCCCCCC(CCCCCCCC)OC(=O)CCCCCCCNCCCn1ccnc1 ; CCCCCCCCC(CC)OC(=O)CCCCCCCN(CCCCCCCC(=O)OC(CCCCCCCC)CCCCCCCC)CCCn1ccnc1]

In a 100 mL round bottom flask connected with condenser, heptadecan-9-yl 8-((3-(1H-imidazol-1-yl)propyl)amino)octanoate (6) (242 mg, 0.478 mmol) and undecan-3-yl 8-bromooctanoate (12) (200 mg, 0.574 mmol) were mixed in ethanol (10 mL), then DIPEA (0.2 mL, 1.196 mmol) was added. The reaction mixture was heated to reflux overnight. Both MS (APCI) and TLC (10% MeOH+1% NH₄OH in CH₂Cl₂) showed the product and some unreacted starting material. The mixture was cooled to room temperature and concentrated. The crude residue was purified by flash chromatography (SiO₂: CH₂Cl₂=100% to 10% of methanol+1% NH₄OH in CH₂Cl₂) and colorless oil was obtained (35 mg, 10%).

¹H NMR (300 MHz, CDCl3): δ ppm 7.45 (s, 1H), 7.04 (s, 1H), 6.90 (s, 1H), 4.82 (m, 2H), 3.97 (t, J=6.8 Hz, 2H), 2.35 (m, 6H), 2.27 (t, J=3.8 Hz, 4H), 1.89 (m, 2H), 1.60-1.48 (m, 14H), 1.30-1.20 (m, 50H), 0.87 (m, 12H). MS (APCI+): 802.8

### 36.9 Synthesis of Heptadecan-9-yl 8-((3-(2-methyl-1H-imidazol-1-yl)propyl)(8-(nonyloxy)-8-oxooctyl)amino)octanoate (Lipid 138, Table 10a)

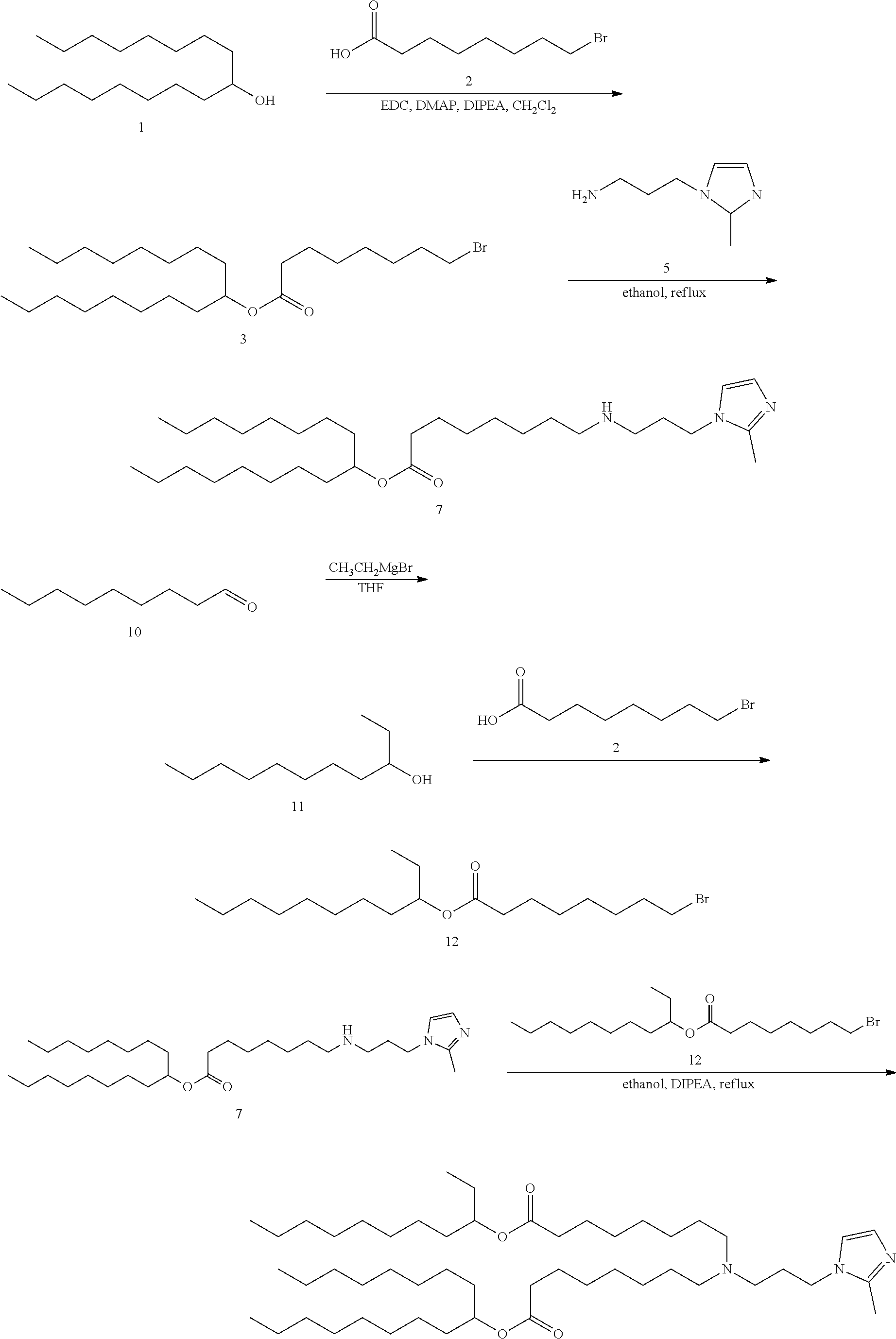

Lipid 138 from Table 10a is synthesized according to the scheme above. Reaction conditions are identical to Lipid 137 with the exception of 3-(2-Methyl-1H-imidazol-1-yl)propan-1-amine as the imidazole amine.

¹H NMR (300 MHz, CDCl3): δ ppm 6.89 (s, 1H), 6.81 (s, 1H), 4.82 (m, 2H), 3.86 (t, J=7.1 Hz, 2H), 2.38-2.3 (m, 9H), 2.27 (t, J=3.8 Hz, 4H), 1.84 (m, 2H), 1.60-1.37 (m, 14H), 1.30-1.20 (m, 50H), 0.87 (m, 12H). MS (APCI+): 816.8 (M+1).

### 36.10 Synthesis of (((2-(2-Methyl-1H-imidazol-1-yl)ethyl)azanediyl)bis(hexane-6,1-diyl) bis(2-hexyldecanoate (Lipid 139, Table 10a)

### 36.10.1 Synthesis of 6-bromohexyl 2-hexyldecanoate (3)

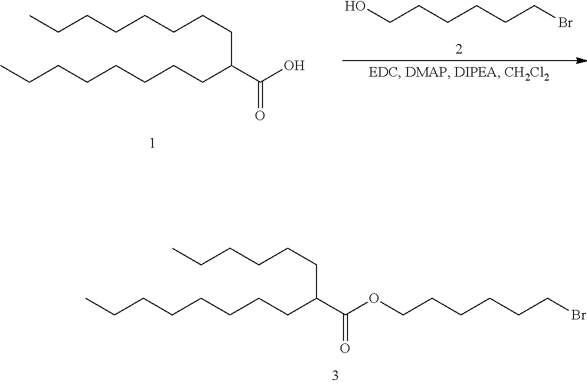 [SMILES: CCCCCCCCC(CCCCCCCC)C(=O)O ; OCCCCCCBr ; CCCCCCCCC(CCCCCC)C(=O)OCCCCCCBr]

To a mixture of 2-hexyldecanoic acid (1) (102 g, 0.398 mol) and 6-bromo-1-hexanol (2) (60 g, 0.331 mol) in CH₂Cl₂ (1 L) was added DMAP (8.1 g, 66 mmol), DIPEA (230 mL, 1.325 mol) and EDC (76 g, 0.398 mol). The reaction was stirred at room temperature overnight. After concentration of the reaction mixture, the crude residue was dissolved in ethyl acetate (1 L), washed with 1N HCl, sat. NaHCO₃, water and Brine. The organic layer was dried over anhydrous Na₂SO₄. The solvent was evaporated and the crude residue was purified by flash chromatography (SiO₂: Hexane=100% to 30% of EtOAc in Hexane) and colorless oil product 3 was obtained (67 g, 48%).

¹H NMR (300 MHz, CDCl₃): δ ppm 4.06 (t, J=6.6 Hz, 2H), 3.4 (t, J=6.8 Hz, 2H), 2.3 (m, 1H), 1.86 (m, 2H), 1.64 (m, 2H), 1.5-1.4 (m, 2H), 1.35-1.2 (m, 26H) 0.87 (t, J=6.7 Hz, 6H).

### 36.10.2 Synthesis of 6-((3-(1H-imidazol-1-yl)butyl)amino)hexyl 2-hexyldecanoate (7a)

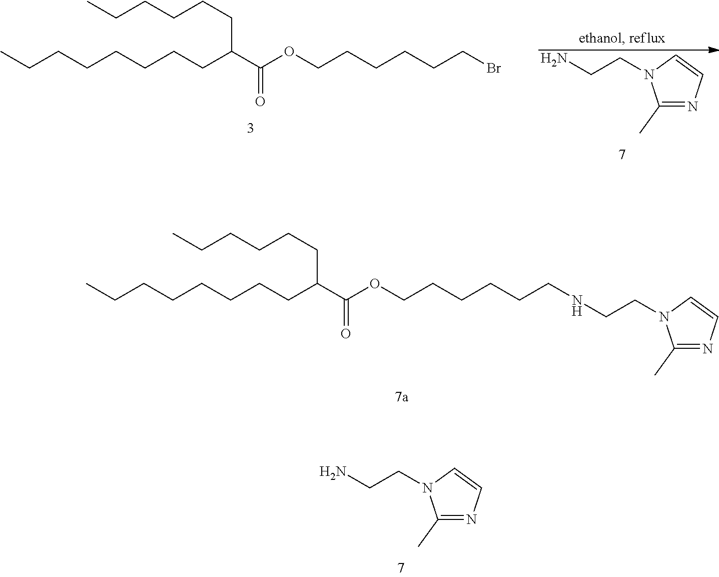

In a 100 mL round bottom flask connected with condenser, 6-bromohexyl 2-hexyldecanoate (3) (1.2 g, 2.87 mmol) and 3-(1H-imidazol-1-yl)butan-1-amine (7) (2 g, 14.37 mmol) were mixed in ethanol (20 mL). The reaction mixture was heated to reflux overnight. MS (APCI) showed the expected product. The mixture was cooled to room temperature and concentrated. The crude residue was purified by flash chromatography (SiO₂: CH₂Cl₂=100% to 10% of methanol+1% NH₄OH in CH₂Cl₂) and colorless oil product 7a was obtained (626 mg, 46%).

¹H NMR (300 MHz, CDCl₃): δ ppm 7.51 (s, 1H), 7.05 (s, 1H), 6.93 (s, 1H), 4.35 (m, 1H), 4.04 (t, J=6.6 Hz, 2H), 2.6-2.4 (m, 4H), 2.29 (m, 1H), 1.94 (td, J=14, 6.8 Hz, 2H), 1.64-1.56 (m, 4H), 1.47 (s, 3H), 1.42-1.20 (m, 29H), 0.86 (m, 6H). MS (APCI⁺): 478.8 (M+1)

### 36.10.2 Synthesis of ((2-(2-Methyl-1H-imidazol-1-yl)ethyl)azanediyl)bis(hexane-6,1-diyl) bis(2-hexyldecanoate

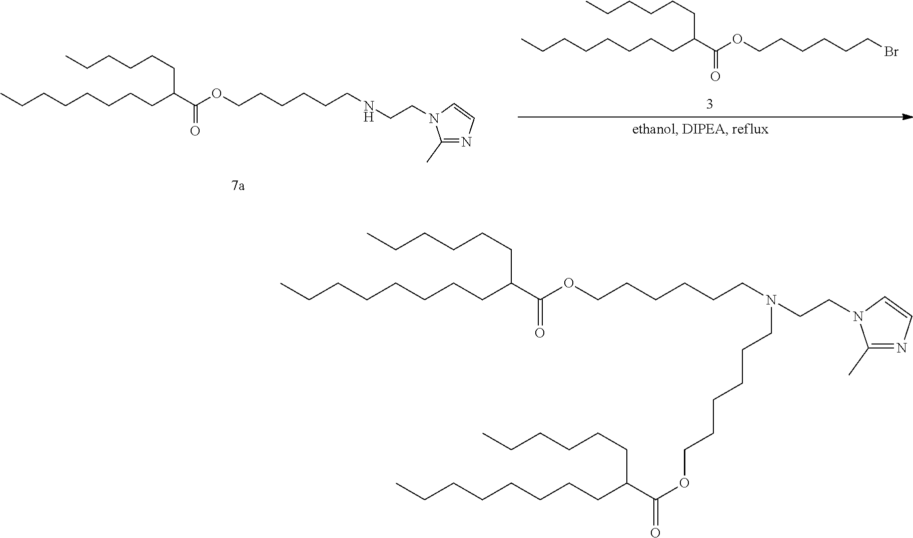 [SMILES: CCCCCCCCC(CCCCCC)C(=O)OCCCCCCNCCn1ccnc1C ; CCCCCCCCC(CCCCCC)C(=O)OCCCCCCBr ; CCCCCCCCC(CCCCCC)C(=O)OCCCCCCN(CCCCCCOC(=O)C(CCCCCC)CCCCCCCC)CCn1ccnc1C]

In a 100 mL round bottom flask connected with condenser, 6-((3-(1H-imidazol-1-yl)butyl)amino)hexyl 2-hexyldecanoate (7a) (626 mg, 1.31 mmol) and 6-bromohexyl 2-hexyldecanoate (3) (550 mg, 1.31 mmol) were mixed in ethanol (20 mL), then DIPEA (0.6 mL, 3.276 mmol) was added. The reaction mixture was heated to reflux overnight. Both MS (APCI) and TLC (10% MeOH+1% NH₄OH in CH₂Cl₂) showed the product and unreacted starting material 7a. The mixture was cooled to room temperature and concentrated. The crude residue was purified by flash chromatography (SiO₂: CH₂Cl₂=100% to 10% of methanol+1% NH₄OH in CH₂Cl₂) and the obtained product was further purified by C18 reverse phase chromatography (H₂O=95% to 0.1% TFA in CH₃CN=100%) colorless oil (TFA salt) was obtained (140 mg, 13%).

¹H-NMR (300 MHz, CDCl₃): δ 6.87 (s, 1H), 6.83 (s, 1H), 4.05 (t, J=6.7 Hz, 4H), 3.84 (t, J=6.9 Hz, 2H), 2.66 (t, J=6.9 Hz, 2H), 2.45-2.20 (m, 6H), 2.37 (s, 3H), 1.65-1.50 (m, 8H), 1.5-1.1 (m, 56H), 0.86 (t, J=6.5 Hz, 12H). MS (APCI⁺): 802.6 (M+1).

### 36.11 Synthesis of (((1-Methyl-1H-imidazol-2-yl)methyl)azanediyl)bis(hexane-6,1-diyl) bis(2-hexyldecanoate) (Lipid 130, Table 10a)

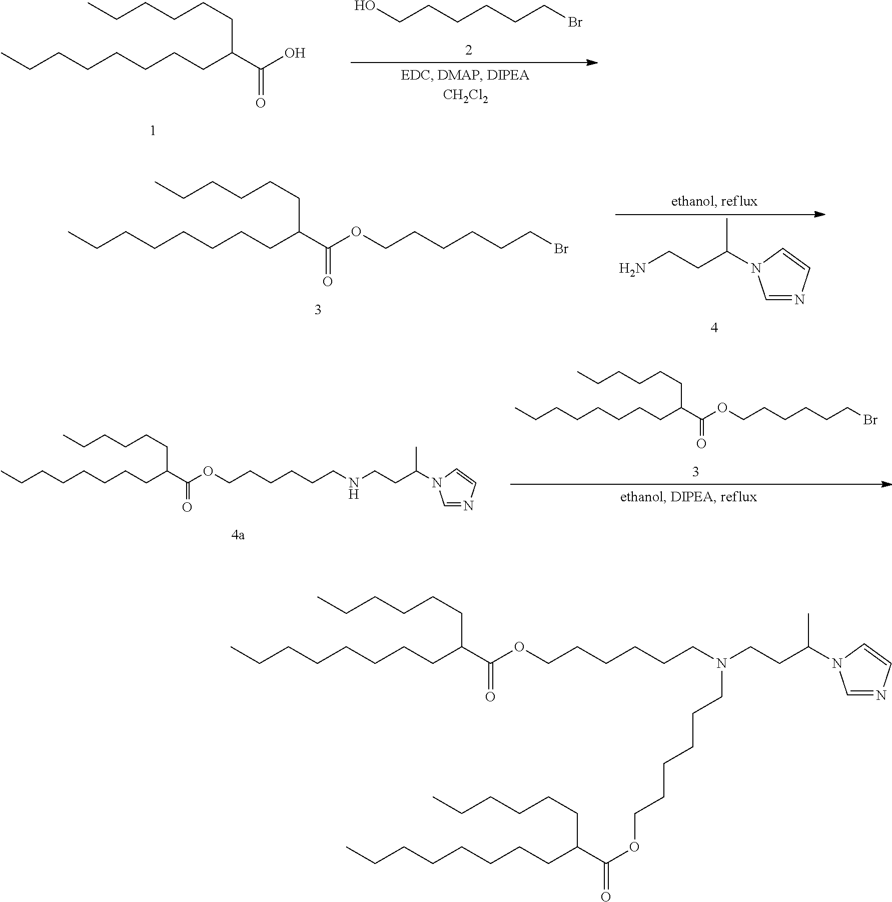

Lipid 130 from Table 10a is synthesized according to the scheme above. Reaction conditions are identical to Lipid 139 with the exception of 3-(1H-imidazol-1-yl)butyl amine as the imidazole amine.

### 36.12 Synthesis of (((1-Methyl-1H-imidazol-2-yl)methyl)azanediyl)bis(hexane-6,1-diyl) bis(2-hexyldecanoate) (Lipid 128, Table 10a)

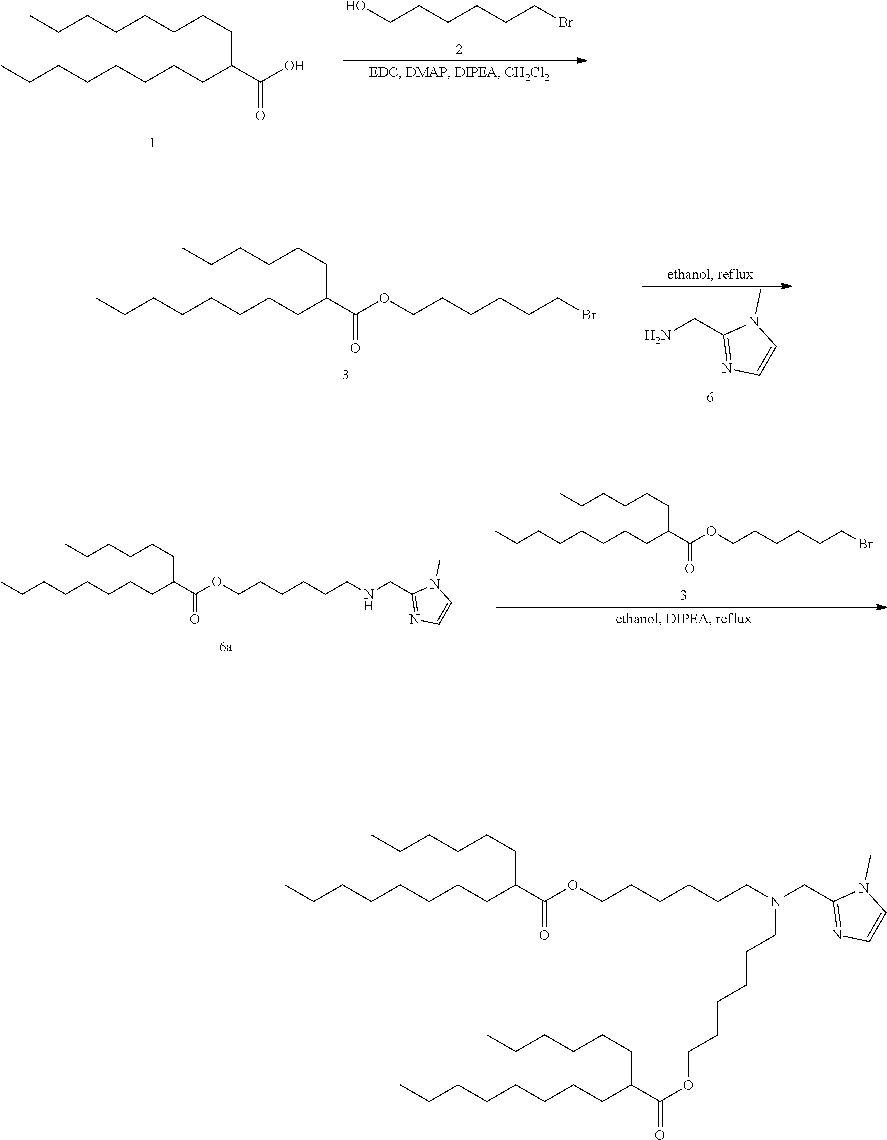

Lipid 128 from Table 10a is synthesized according to the scheme above. Reaction conditions are identical to Lipid 139 with the exception of 1-Methyl-1H-imidazol-2-yl)methyl amine as the imidazole amine.

¹H-NMR (300 MHz, CDCl₃): δ 6.89 (d, J=1.4 Hz, 1H), 6.81 (d, J=1.4 Hz, 1H), 4.03 (t, J=6.7 Hz, 4H), 3.68 (s, 3H), 3.62 (s, 2H), 2.45-2.20 (m, 6H), 1.65-1.50 (m, 8H), 1.5-1.35 (m, 8H), 1.35-1.10 (m, 48H), 0.86 (t, J=6.5 Hz, 12H). MS (APCI⁺): 787.6 (M+1).

### Example 37

Lipid Nanoparticle Formulation with Circular RNA

Lipid Nanoparticles (LNPs) were formed using a Precision Nanosystems Ignite instrument with a ‘NextGen’ mixing chamber. Ethanol phase contained ionizable Lipid 26 from Table 10a, DSPC, Cholesterol, and DSPE-PEG 2000 (Avanti Polar Lipids Inc.) at a weight ratio of 16:1:4:1 or 62:4:33:1 molar ratio was combined with an aqueous phase containing circular RNA and 25 mM sodium acetate buffer at pH 5.2. A 3:1 aqueous to ethanol mixing ratio was used. The formulated LNP then were dialyzed in 1 L of water and exchanged 2 times over 18 hours. Dialyzed LNPs were filtered using 0.2 μm filter. Prior to in vivo dosing, LNPs were diluted in PBS. LNP sizes were determined by dynamic light scattering. A cuvette with 1 mL of 20 μg/mL LNPs in PBS (pH 7.4) was measured for Z-average using the Malvern Panalytical Zetasizer Pro. The Z-average and polydispersity index were recorded.

39.1 Formulation of Lipids 26 and 27 from Table 10a

Lipid Nanoparticles (LNPs) were formed using a Precision Nanosystems Ignite instrument with a ‘NextGen’ mixing chamber. Ethanol phase contained ionizable Lipid 26 or Lipid 27 from Table 10a, DOPE, Cholesterol, and DSPE-PEG 2000 (Avanti Polar Lipids Inc.) at a weight ratio of 16:1:4:1 or 62:4:33:1 molar ratio was combined with an aqueous phase containing circular RNA and 25 mM sodium acetate buffer at pH 5.2. A 3:1 aqueous to ethanol mixing ratio was used. The formulated LNPs were then dialyzed in 1 L of water and exchanged 2 times over 18 hours. Dialyzed LNPs were filtered using 0.2 μm filter. Prior to in vivo dosing, LNPs were diluted in PBS. LNP sizes were determined by dynamic light scattering. A cuvette with 1 mL of 20 μg/mL LNPs in PBS (pH 7.4) was measured for Z-average using the Malvern Panalytical Zetasizer Pro. The Z-average and polydispersity index were recorded.

39.2 Formulation of Lipids 53 and 54 from Table 10a

Lipid Nanoparticles (LNPs) were formed using a Precision Nanosystems Ignite instrument with a ‘NextGen’ mixing chamber. Ethanol phase contained ionizable Lipid 53 or 54 of Table 10a, DOPE, Cholesterol, and DSPE-PEG 2000 (Avanti Polar Lipids Inc.) at a molar ratio of 50:10:38.5:1.5 was combined with an aqueous phase containing circular RNA and 25 mM sodium acetate buffer at pH 5.2. A 3:1 aqueous to ethanol mixing ratio was used. The formulated LNPs were then dialyzed in 1 L of 1×PBS and exchanged 2 times over 18 hours. Dialyzed LNPs were filtered using 0.2 μm filter. Prior to in vivo dosing, LNPs were diluted in PBS. LNP sizes were determined by dynamic light scattering. A cuvette with 1 mL of 20 μg/mL LNPs in PBS (pH 7.4) was measured for Z-average using the Malvern Panalytical Zetasizer Pro. The Z-average and polydispersity index were recorded.

LNP zeta potential was measured using the Malvern Panalytical Zetasizer Pro. A mixture containing 200 μL of the particle solution in water and 800 μL of distilled RNAse-free water with a final particle concentration of 400 μg/mL was loaded into a zetasizer capillary cell for analysis.

RNA encapsulation was determined using a Ribogreen assay. Nanoparticle solutions were diluted in tris-ethylenediaminetetraacetic acid (TE) buffer at a theoretical oRNA concentration of 2 μg/mL. Standard oRNA solutions diluted in TE buffer were made ranging from 2 μg/mL to 0.125 μg/mL. The particles and standards were added to all wells and a second incubation was performed (37° C. at 350 rpm for 3 minutes). Fluorescence was measured using a SPECTRAmax® GEMINI XS microplate spectrofluorometer. The concentration of circular RNA in each particle solution was calculated using the standard curve. The encapsulation efficiency was calculated from the ratio of oRNA detected between lysed and unlysed particles.

TABLE 36a
Characterization of LNPs

| | | | Encapsulation | Zeta Data |
|---|---|---|---|---|
| Ionizable Lipid | Size (nm) | PDI | Efficiency (%) | Potential (mV) |
| 22-S14 | 88 | 0.09 | 96 | 3.968 |
| 93-S14 | 119 | 0.02 | 96 | −6.071 |
| Lipid 26, Table 10a | 86 | 0.08 | 92 | −15.24 |

TABLE 36b
Characterization of LNPs

| Ionizable Lipid | Z-Average(nm) | PDI | RNA Entrapment(%) |
|---|---|---|---|
| 22-S14 | 64 | 0.05 | 97 |
| 93-S14 | 74 | 0.04 | 95 |
| Lipid 26, Table 10a | 84 | 0.04 | 96 |

### Example 38

### In Vivo Analysis

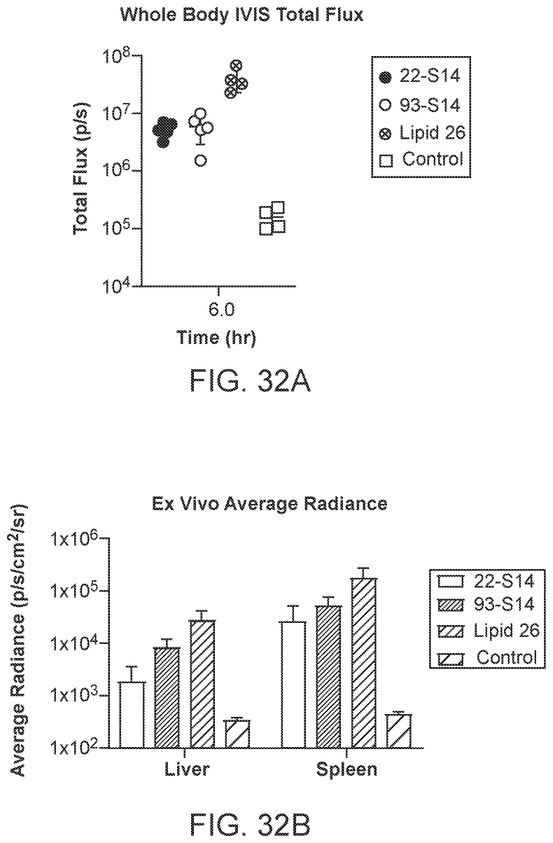

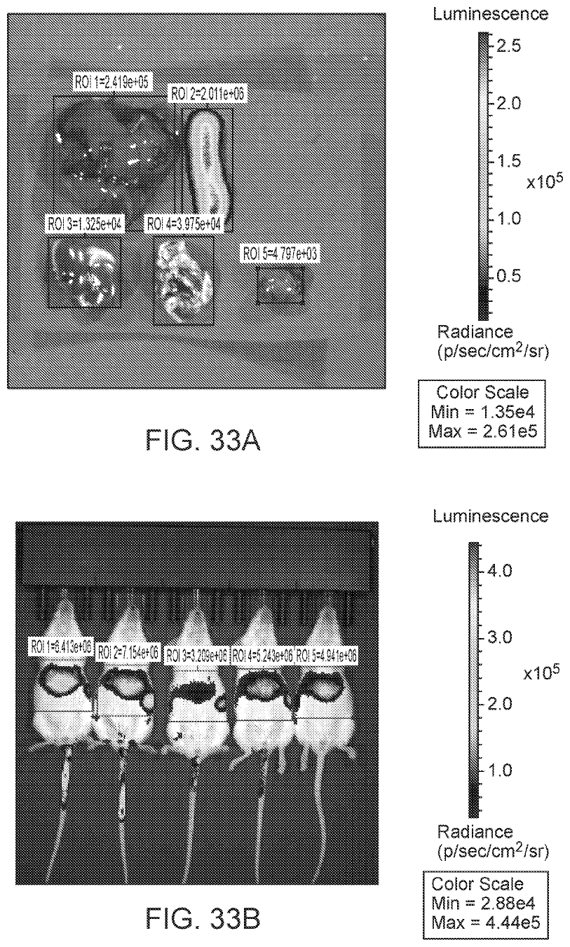

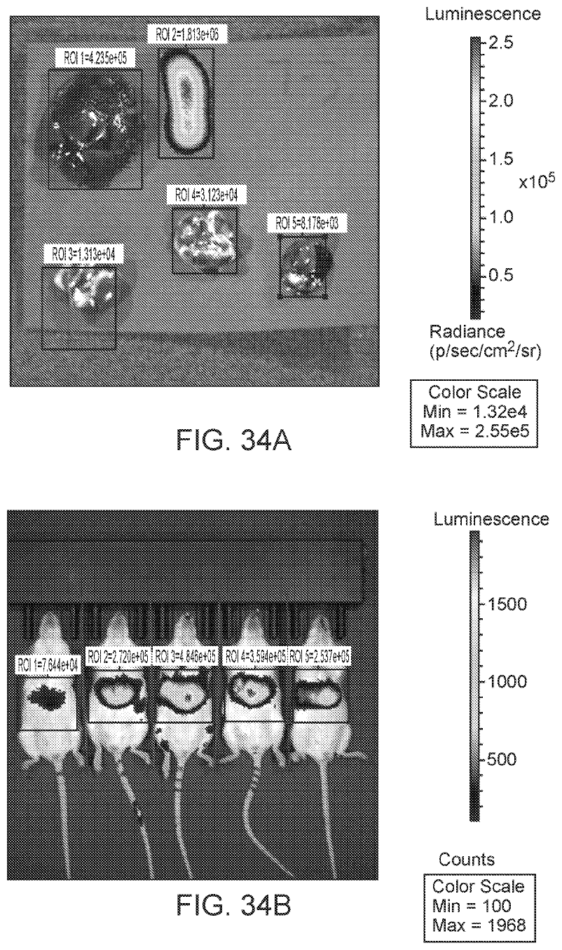

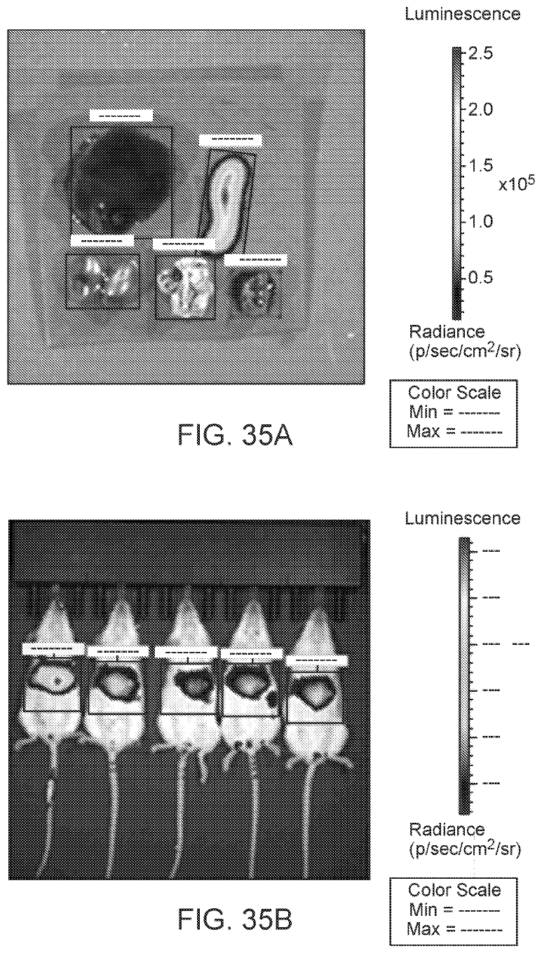

Female CD-1 or female c57BL/6J mice ranging from 22-25 g were dosed at 0.5 mg/kg RNA intravenously. Six hours after injection, mice were injected intraperitoneally with 200 μL of D-luciferin at 15 mg/mL concentration. 5 minutes after injection, mice were anesthetized using isoflurane, and placed inside the IVIS Spectrum In Vivo Imaging System (Perkin Elmer) with dorsal side up. Whole body total IVIS flux of Lipids 22-S14, 93-S14, Lipid 26 (Table 10a) is presented in FIG. 32A. Post 10 minutes injection, mice were scanned for luminescence. Mice were euthanized and organs were extracted within 25 minutes of luciferin injection to scan for luminescence in liver, spleen, kidneys, lungs, and heart. Images (FIGS. 33A-B, 34A-B, 35A-B) were analyzed using Living Images (Perkin Elmer) software. Regions of interest were drawn to obtain flux and average radiance and analyzed for biodistribution of protein expression (FIG. 32A-B).

FIG. 32A illustrates the increased whole-body total flux observed from luciferase oRNA with Lipid 26 (Table 10a) LNPs compared to LNPs made with lipids 22-S14 and 93-S14. FIG. 32B shows the ex vivo IVIS analysis of tissues further highlighting the increased overall expression with Lipid 26 (Table 10a) while maintaining the desired spleen to liver ratios observed with lipids 22-S14 and 93-S14 despite the significant structural changes designed to improve expression. These data highlight the improvements afforded by Lipid 26 (Table 10a) compared to previously reported lipids.

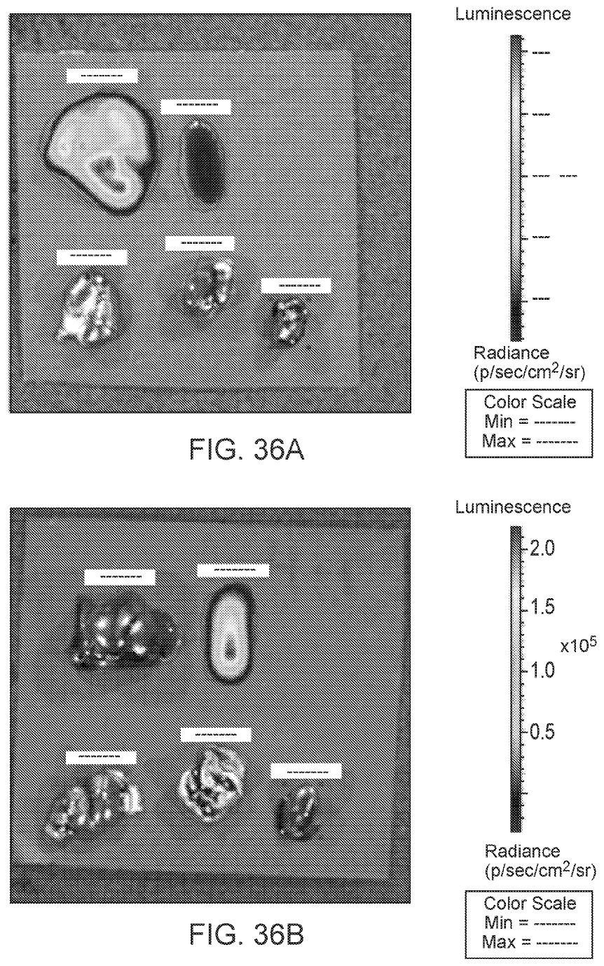

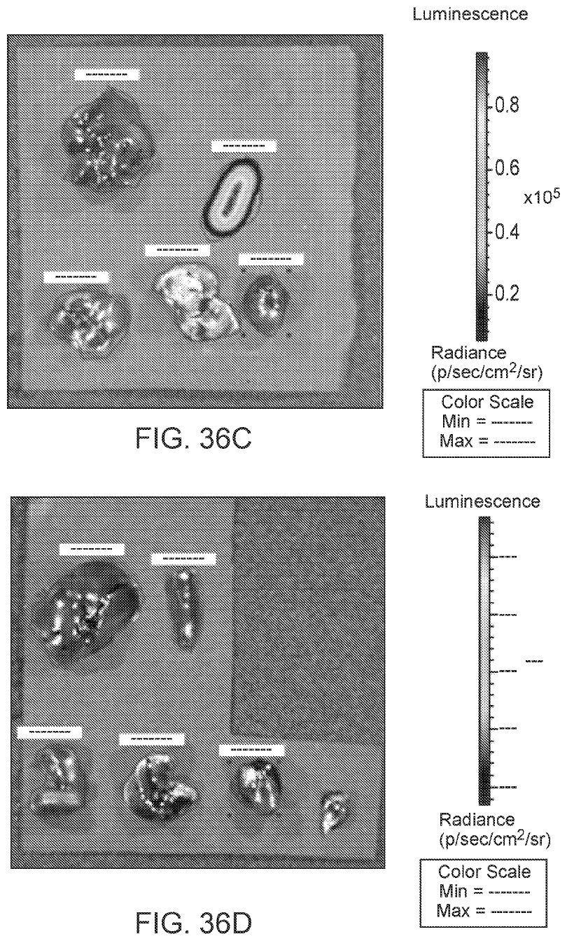

Similar analysis as described above was also performed with oRNA encapsulated in LNPs formed with Lipid 15 from Table 10b or Lipid 53 or 54 from Table 10a. FIGS. 36A-C show the ex vivo IVIS analysis of tissues, respectively highlighting the overall expression with Lipid 15, 53, and 54 while maintaining the desired spleen to liver ratios despite the significant structural changes designed to improve expression. FIG. 36D shows the results for PBS control. These data demonstrates the improvements afforded by Lipids 15, 53, and 54 from Table 10a compared to previously reported lipids such as 93-S14 and 22-S14.

### Example 39

### Delivery of Luciferase

Human peripheral blood mononuclear cells (PBMCs) (Stemcell Technologies) were transfected with lipid nanoparticles (LNP) encapsulating firefly luciferase (f.luc) circular RNA and examined for luciferase expression. PBMCs from two different donors were incubated in vitro with five different LNP compositions, containing circular RNA encoding for firefly luciferase (200 ng), at 37° C. in RPMI, 2% human serum, IL-2 (10 ng/mL), and 50 uM BME. PBMCs incubated without LNP were used as a negative control. After 24 hours, the cells were lysed and analyzed for firefly luciferase expression based on bioluminescence (Promega BrightGlo).

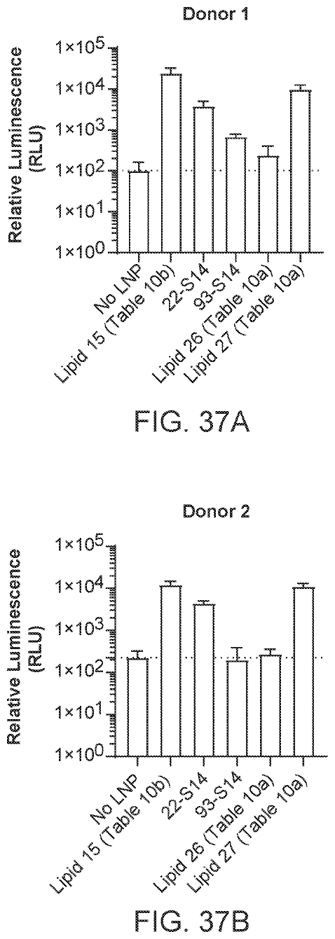

Representative data are presented in FIGS. 37A and 37B, showing that that the tested LNPs are capable of delivering circular RNA into primary human immune cells resulting in protein expression.

### Example 40

### In Vitro Delivery of Green Fluorescent Protein (GFP) or Chimeric Antigen Receptor (CAR)

Human PBMCs (Stemcell Technologies) were transfected with LNP encapsulating GFP and examined by flow cytometry. PBMCs from five different donors (PBMC A-E) were incubated in vitro with one LNP composition, containing circular RNA encoding either GFP or CD19-CAR (200 ng), at 37° C. in RPMI, 2% human serum, IL-2 (10 ng/mL), and 50 uM BME. PBMCs incubated without LNP were used as a negative control. After 24, 48, or 72 hours post-LNP incubation, cells were analyzed for CD3, CD19, CD56, CD14, CD11b, CD45, fixable live dead, and payload (GFP or CD19-CAR).

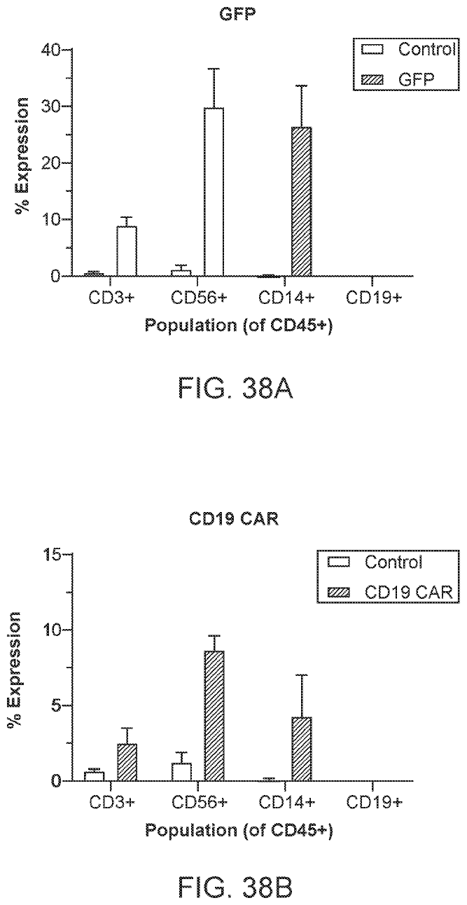

Representative data are presented in FIGS. 38A and 38B, showing that the tested LNP is capable of delivering circular RNA into primary human immune cells resulting in protein expression.

### Example 41

### Multiple IRES Variants can Mediate Expression of Murine CD19 CAR In Vitro

Multiple circular RNA constructs, encoding anti-murine CD19 CAR, contains unique IRES sequences and were lipotransfected into 1C1C7 cell lines. Prior to lipotransfection, 1C1C7 cells are expanded for several days in complete RPMI Once the cells expanded to appropriate numbers, 1C1C7 cells were lipotransfected (Invitrogen RNAiMAX) with four different circular RNA constructs. After 24 hours, 1C1C7 cells were incubated with His-tagged recombinant murine CD19 (Sino Biological) protein, then stained with a secondary anti-His antibody. Afterwards, the cells were analyzed via flow cytometry.

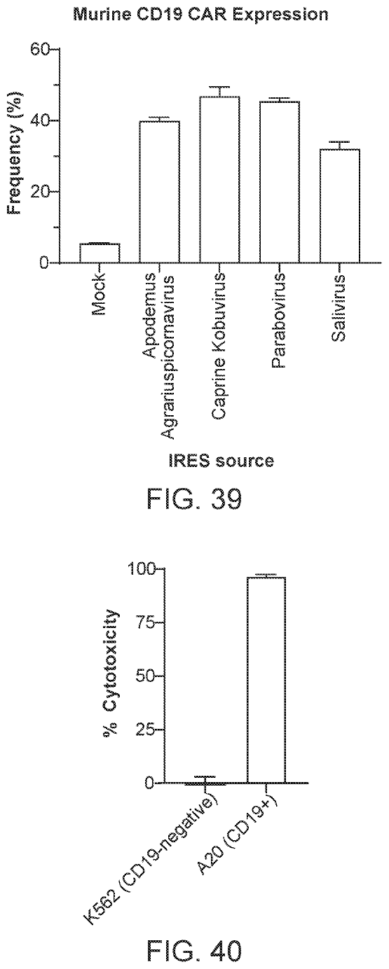

Representative data are presented in FIGS. 39, showing that IRES sourced from the indicated virus (apodemus agrarius picornavirus, caprine kobuvirus, parabovirus, and salivirus) are capable of driving expression of an anti-mouse CD19 CAR in murine T cells.

### Example 42

### Murine CD19 CAR Mediates Tumor Cell Killing In Vitro

Circular RNA encoding anti-mouse CD19 CAR were electroporated into murine T cells to evaluate CAR-mediated cytotoxicity. For electroporation, T cells were electroporated with circular RNA encoding anti-mouse CD19 CAR using ThermoFisher's Neon Transfection System then rested overnight. For the cytotoxicity assay, electroporated T cells were co-cultured with Fluc+ target and non-target cells at 1:1 ratio in complete RPMI containing 10% FBS, IL-2 (10 ng/mL), and 50 uM BME and incubated overnight at 37° C. Cytotoxicity was measured using a luciferase assay system 24 hours post-co-culture (Promega Brightglo Luciferase System) to detect lysis of Fluc+ target and non-target cells. Values shown are calculated relative to the untransfected mock signal.

Representative data are presented in FIG. 40, showing that an anti-mouse CD19 CAR expressed from circular RNA is functional in murine T cells in vitro.

### Example 43

Functional Depletion of B Cells with a Lipid Encapsulated Circular RNA Encoding Murine CD19 CAR

C57BL/6J mice were injected with LNP formed with Lipid 15 in Table 10b, encapsulating circular RNA encoding anti-murine CD19 CAR. As a control, Lipid 15 in Table 10b encapsulating circular RNA encoding firefly luciferase (f.Luc) were injected in different group of mice. Female C57BL.6J, ranging from 20-25 g, were injected intravenously with 5 doses of 0.5 mg/kg of LNP, every other day. Between injections, blood draws were analyzed via flow cytometry for fixable live/dead, CD45, TCRvb, B220, CD11b, and anti-murine CAR. Two days after the last injection, spleens were harvested and processed for flow cytometry analysis. Splenocytes were stained with fixable live/dead, CD45, TCRvb, B220, CD11b, NK1.1, F4/80, CD11 c, and anti-murine CAR. Data from mice injected with anti-murine CD19 CAR LNP were normalized to mice that received f.Luc LNP.

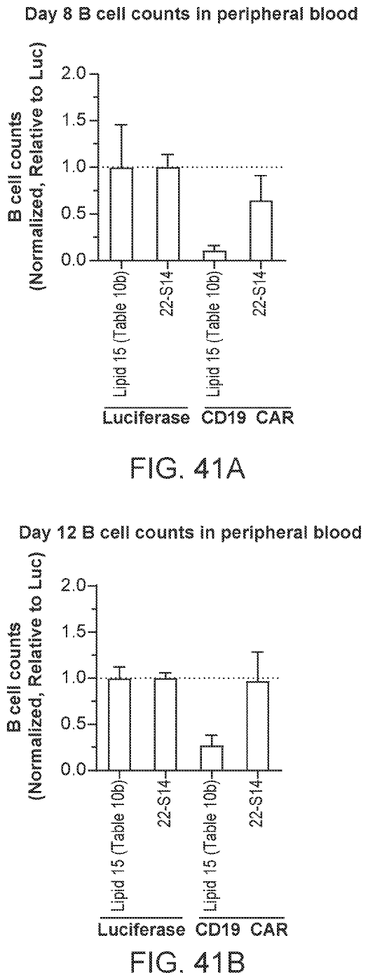

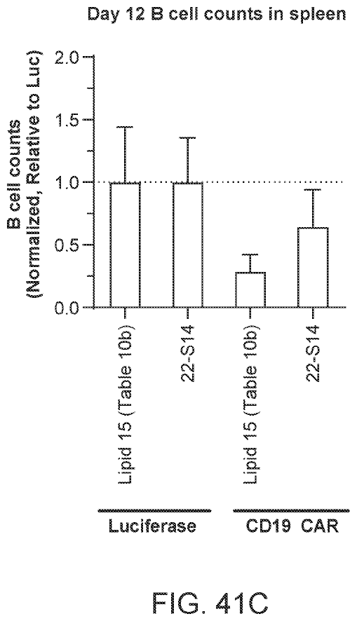

Representative data are presented in FIGS. 41A, 41B, and 41C, showing that an anti-mouse CD19 CAR expressed from circular oRNA delivered in vivo with LNPs is functional in murine T cells in vivo.

### Example 44

CD19 CAR Expressed from Circular RNA has Higher Yield and Greater Cytotoxic Effect Compared to that Expressed from mRNA

Circular RNA encoding anti-CD19 chimeric antigen receptor, which includes, from N-terminus to C-terminus, a FMC63-derived scFv, a CD8 transmembrane domain, a 4-1BB costimulatory domain, and a CD3intracellular domain, were electroporated into human peripheral T cells to evaluate surface expression and CAR-mediated cytotoxicity. For comparison, circular RNA-electroporated T cells were compared to mRNA-electroporated T cells in this experiment. For electroporation, CD3+ T cells were isolated from human PBMCs using commercially available T cell isolation kits (Miltenyi Biotec) from donor human PBMCs. After isolation, T cells were stimulated with anti-CD3/anti-CD28 (Stemcell Technologies) and expanded over 5 days at 37° C. in complete RPMI containing 10% FBS, IL-2 (10 ng/mL), and 50 uM BME. Five days post stimulation, T cells were electroporated with circular RNA encoding anti-human CD19 CAR using ThermoFisher's Neon Transfection System and then rested overnight. For the cytotoxicity assay, electroporated T cells were co-cultured with Fluc+ target and non-target cells at 1:1 ratio in complete RPMI containing 10% FBS, IL-2 (10 ng/mL), and 50 uM BME and incubated overnight at 37° C. Cytotoxicity was measured using a luciferase assay system 24 hours post-co-culture (Promega Brightglo Luciferase System) to detect lysis of Fluc+ target and non-target cells. Furthermore, an aliquot of electroporated T cells were taken and stained for live dead fixable staining, CD3, CD45, and chimeric antigen receptors (FMC63) at the day of analysis.

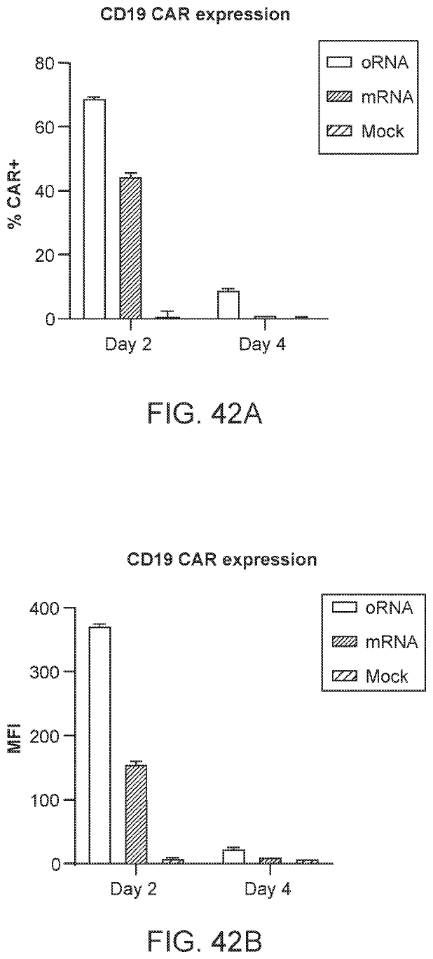

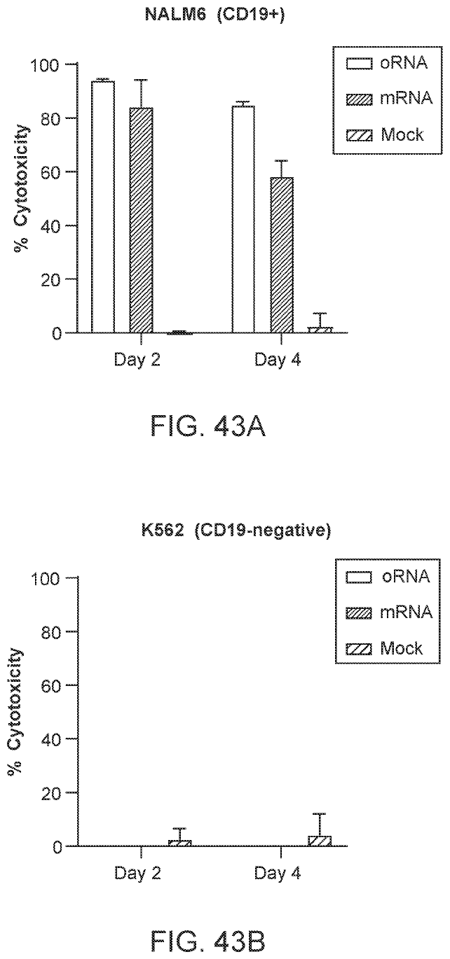

Representative data are presented in FIGS. 42 and 43. FIGS. 42A and 42B show that an anti-human CD19 CAR expressed from circular RNA is expressed at higher levels and longer than an anti-human CD19 CAR expressed from linear mRNA. FIGS. 43A and 43B show that an anti-human CD19 CAR expressed from circular RNA is exerts a greater cytotoxic effect relative to anti-human CD19 CAR expressed from linear mRNA.

### Example 45

Functional Expression of Two CARs from a Single Circular RNA

Circular RNA encoding chimeric antigen receptors were electroporated into human peripheral T cells to evaluate surface expression and CAR-mediated cytotoxicity. The purpose of this study is to evaluate if circular RNA encoding for two CARs can be stochastically expressed with a 2A (P2A) or an IRES sequence. For electroporation, CD3+ T cells were commercially purchased (Cellero) and stimulated with anti-CD3/anti-CD28 (Stemcell Technologies) and expanded over 5 days at 37° C. in complete RPMI containing 10% FBS, IL-2 (10 ng/mL), and 50 uM BME. Four days post stimulation, T cells were electroporated with circular RNA encoding anti-human CD19 CAR, anti-human CD19 CAR-2A-anti-human BCMA CAR, and anti-human CD19 CAR-IRES-anti-human BCMA CAR using ThermoFisher's Neon Transfection System then rested overnight. For the cytotoxicity assay, electroporated T cells were co-cultured with Fluc+K562 cells expressing human CD19 or BCMA antigens at 1:1 ratio in complete RPMI containing 10% FBS, IL-2 (10 ng/mL), and 50 uM BME and incubated overnight at 37° C. Cytotoxicity was measured using a luciferase assay system 24 hours post-co-culture (Promega BrightGlo Luciferase System) to detect lysis of Fluc+ target cells.

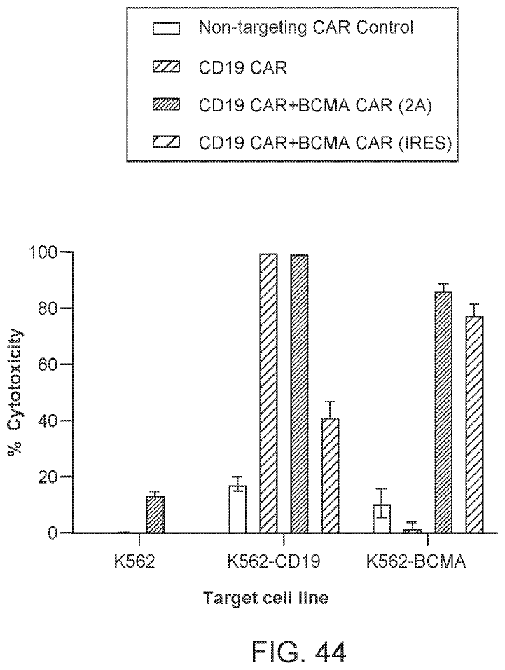

Representative data are presented in FIG. 44, showing that two CARs can be functionally expressed from the same circular RNA construct and exert cytotoxic effector function.

### Example 46

### In Vivo Circular RNA Transfection Using Cre Reporter Mice

Circular RNAs encoding Cre recombinase (Cre) are encapsulated into lipid nanoparticles as previously described. Female, 6-8 week old B6.Cg-Gt(ROSA)26Sortm9(CAG-tdTomato)Hze/J (Ai9) mice were dosed with lipid nanoparticles at 0.5 mg/kg RNA intravenously. Fluorescent tdTomato protein was transcribed and translated in Ai9 mice upon Cre recombination, meaning circular RNAs have been delivered to and translated in tdTomato+ cells. After 48 hr, mice were euthanized and the spleens were harvested, processed into a single cell suspension, and stained with various fluorophore-conjugated antibodies for immunophenotyping via flow cytometry.

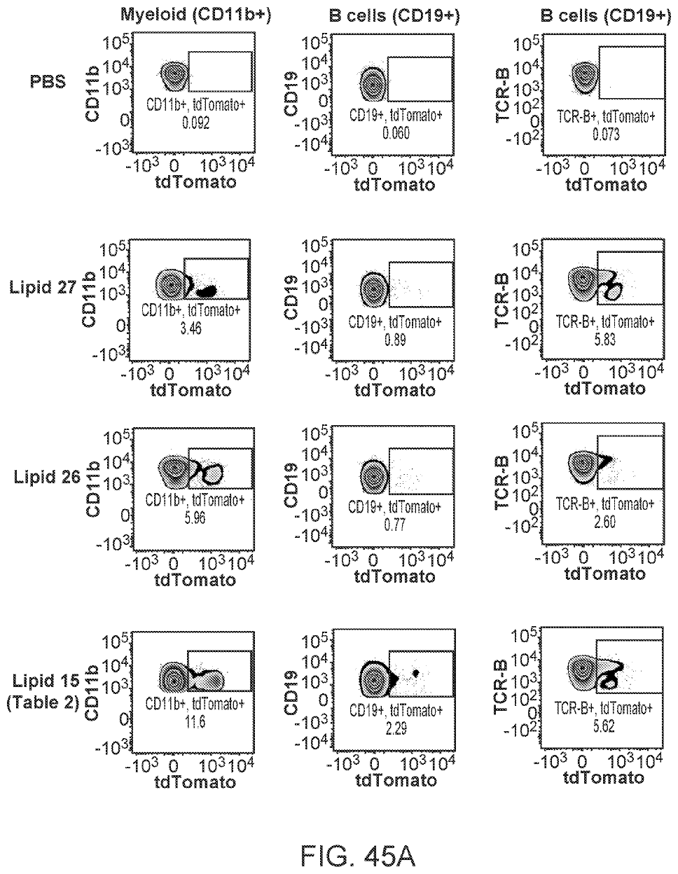

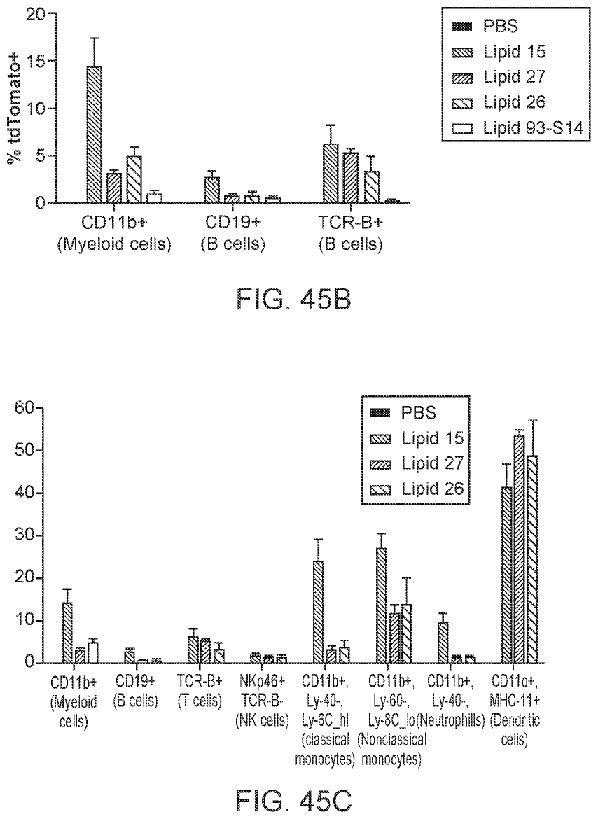

FIG. 45A shows representative FACS plots with frequencies of tdTomato expression in various spleen immune cell (CD45+, live) subsets, including total myeloid (CD11b+), B cells (CD19+), and T cells (TCR-B+) following treatment with LNPs formed with Lipid 27 or 26 from Table 10a or Lipid 15 from Table 10b. Ai9 mice injected with PBS represented background tdTomato fluorescence. FIG. 45B quantifies the proportion of myeloid cells, B cells, and T cells expressing tdTomato (mean+std. dev., n=3), which is equivalent to the proportion of each cell population which has been successfully transfected with Cre circular RNA. LNPs made with Lipids 27 and 26 from Table 10a exhibit significantly higher myeloid and T cell transfection compared with Lipid 93-S14, highlighting the improvements conferred by lipid structural modifications.

FIG. 45C illustrates the proportion of additional splenic immune cell populations expressing tdTomato with Lipids 27 and 26 from Table 10a (mean+std. dev., n=3), which also include NK cells (NKp46+, TCR-B−), classical monocytes (CD11b+, Ly-6G−, Ly-6C_hi), nonclassical monocytes (CD11b+, Ly-6G−, Ly-6C_lo), neutrophils (CD11b+, Ly-6G+), and dendritic cells (CD11c+, MHC-II+). These experiments demonstrate that LNPs made with Lipids 27 and 26 from Table 10a and Lipid 15 from Table 10b are effective at delivering circular RNAs to many splenic immune cell subsets in mice and lead to successful protein expression from the circular RNA in those cells.

### Example 47

### Example 47A: Built-In polyA Sequences and Affinity-Purification to Produce Immune-Silent Circular RNA

PolyA sequences (20-30 nt) were inserted into the 5′ and 3′ ends of the RNA construct (precursor RNA with built-in polyA sequences in the introns). Precursor RNA and introns can alternatively be polyadenylated post-transcriptionally using, e.g., E coli. polyA polymerase or yeast polyA polymerase, which requires the use of an additional enzyme.

Circular RNA in this example was circularized by in vitro transcription (IVT) and affinity-purified by washing over a commercially available oligo-dT resin to selectively remove polyA-tagged sequences (including free introns and precursor RNA) from the splicing reaction. The IVT was performed with a commercial IVT kit (New England Biolabs) or a customerized IVT mix (Orna Therapeutics), containing guanosine monophosphate (GMP) and guanosine triphosphate (GTP) at different ratios (GMP:GTP=8, 12.5, or 13.75). In some embodiments, GMP at a high GMP:GTP ratio may be preferentially included as the first nucleotide, yielding a majority of monophosphate-capped precursor RNAs. As a comparison, the circular RNA product was alternatively purified by the treatment with Xrn1, Rnase R, and Dnase I (enzyme purification).

Immunogenicity of the circular RNAs prepared using the affinity purification or enzyme purification process were then assessed. Briefly, the prepared circular RNAs were transfected into A549 cells. After 24 hours, the cells were lysed and interferon beta-1 induction relative to mock samples was measured by qPCR. 3p-hpRNA, a triphosphorylated RNA, was used as a positive control.

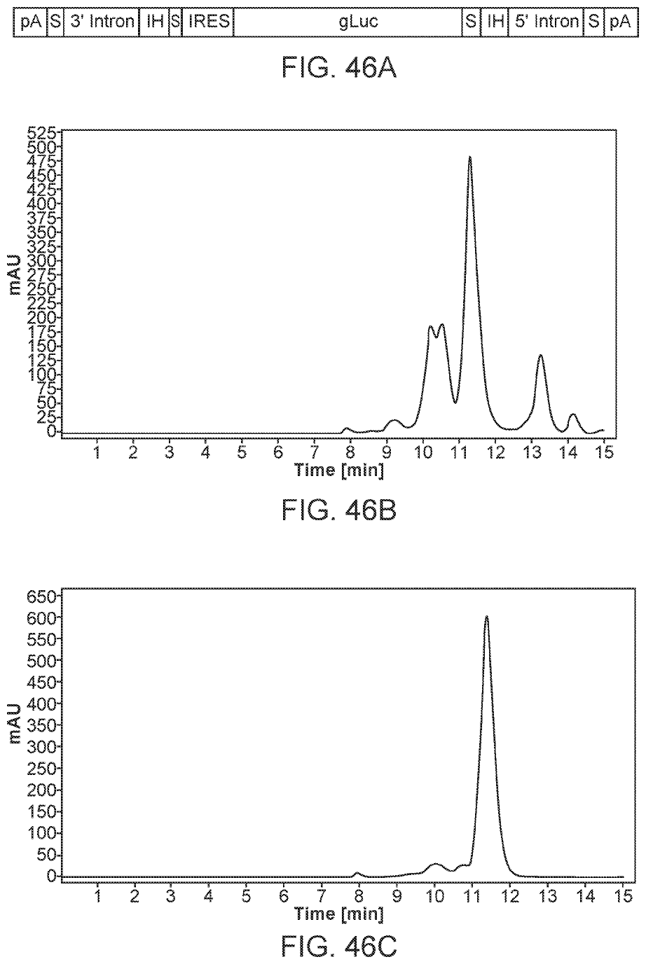

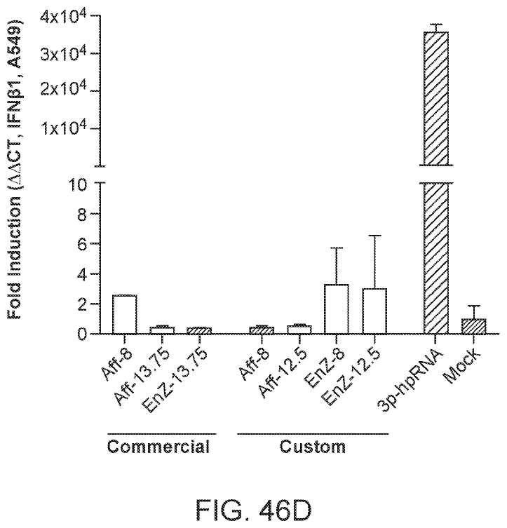

FIGS. 46B and 46C show that the negative selection affinity purification removes non-circular products from splicing reactions when polyA sequences are included on elements that are removed during splicing and present in unspliced precursor molecules. FIG. 46D shows circular RNAs prepared with tested IVT conditions and purification methods are all immunoquiescent. These results suggest the negative selection affinity purification is equivalent or superior to enzyme purification for circular RNA purification and that customized circular RNA synthesis conditions (IVT conditions) may reduce the reliance on GMP excess to achieve maximal immunoquiescence.

### Example 47B: Dedicated Binding Site and Affinity Purification for Circular RNA Production

Instead of polyA tags, one can include specifically design sequences (DBS, dedicated binding site).

Instead of a polyA tag, a dedicated binding site (DBS), such as a specifically designed complementary oligonucleotide that can bind to a resin, may be used to selectively deplete precursor RNA and free introns. In this example, DBS sequences (30 nt) were inserted into the 5′ and 3′ ends of the precursor RNA. RNA was transcribed and the transcribed product was washed over a custom complementary oligonucleotide linked to a resin.

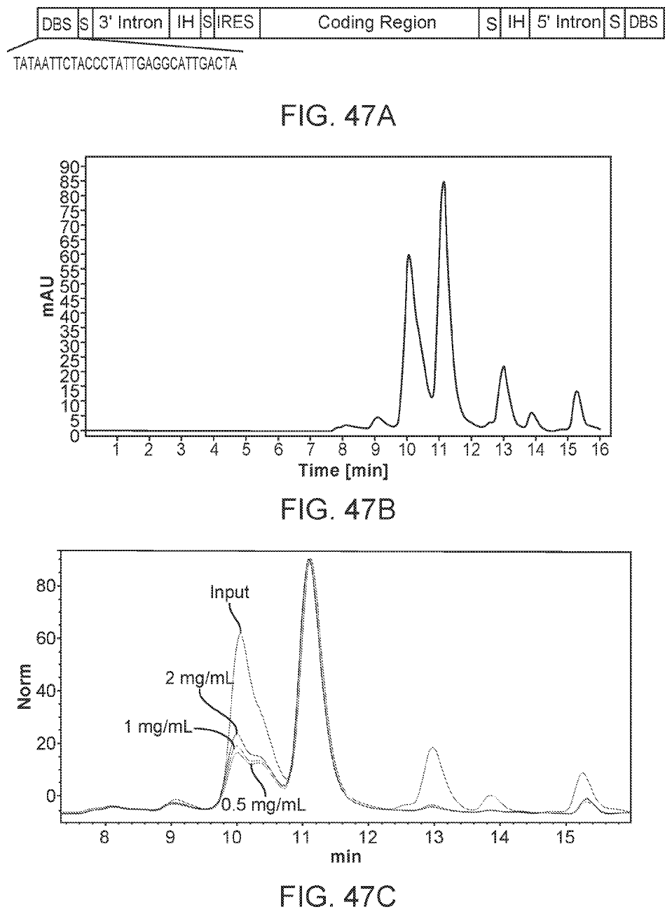

FIGS. 47B and 47C demonstrates that including the designed DBS sequence in elements that are removed during splicing enables the removal of unspliced precursor RNA and free intron components in a splicing reaction, via negative affinity purification.

### Example 47C: Production of a Circular RNA Encoding Dystrophin

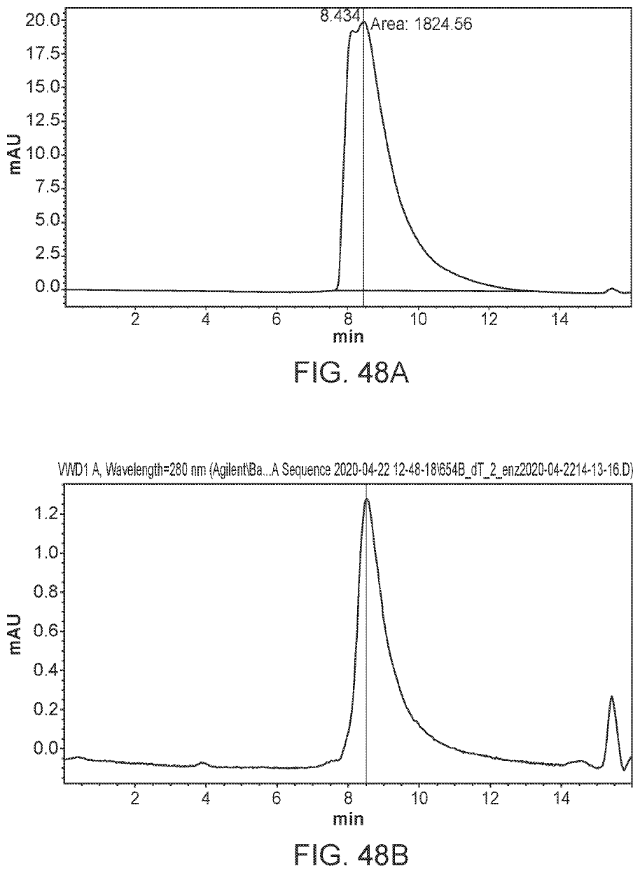

A 12 kb 12,000 nt circular RNA encoding dystrophin was produced by in vitro transcription of RNA precursors followed by enzyme purification using a mixture of Xrn1, DNase 1, and RNase R to degrade remaining linear components. FIG. 48 shows that the circular RNA encoding dystrophin was successfully produced.

### Example 48

### 5′ Spacer Between 3′ Intron Fragment and the IRES Improves Circular RNA Expression

Expression level of purified circRNAs with different 5′ spacers between the 3′ intron fragment and the IRES in Jurkat cells were compared. Briefly, luminescence from secreted Gaussia luciferase in supernatant was measured 24 hours after electroporation of 60,000 cells with 250 ng of each RNA.

Additionally, stability of purified circRNAs with different 5′ spacers between the 3′ intron fragment and the IRES in Jurkat cells were compared. Briefly, luminescence from secreted Gaussia luciferase in supernatant was measured over 2 days after electroporation of 60,000 cells with 250 ng of each RNA and normalized to day 1 expression.

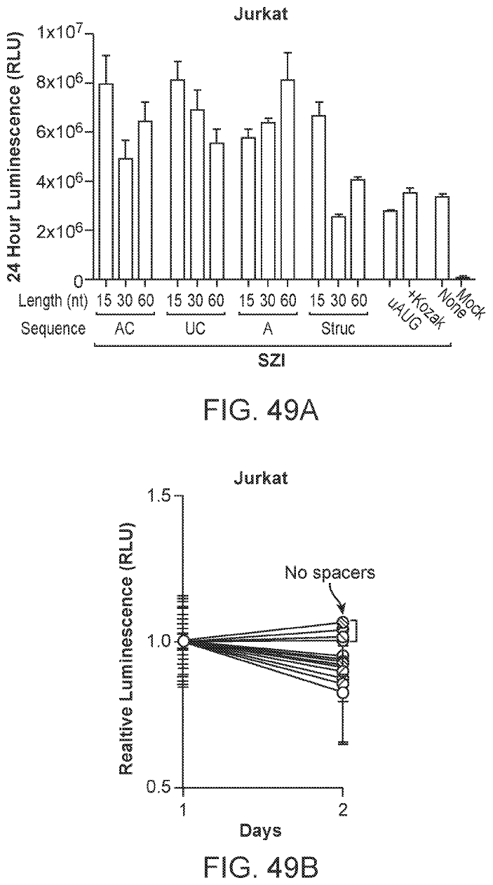

The results are shown in FIGS. 49A and 49B, indicating that adding a spacer can enhance IRES function and the importance of sequence identity and length of the added spacer. A potential explanation is that the spacer is added right before the IRES and likely functions by allowing the IRES to fold in isolation from other structured elements such as the intron fragments.

### Example 49

This example describes deletion scanning from 5′ or 3′ end of the caprine kobuvirus IRES. IRES borders are generally poorly characterized and require empirical analysis, and this example can be used for locating the core functional sequences required for driving translation. Briefly, circular RNA constructs were generated with truncated IRES elements operably linked to a Gaussia luciferase coding sequence. The truncated IRES elements had nucleotide sequences of the indicated lengths removed from the 5′ or 3′ end. Luminescence from secreted Gaussia luciferase in supernatant was measured 24 and 48 hours after electroporation of primary human T cells with RNA. Stability of expression was calculated as the ratio of the expression level at the 48-hour time point relative to that at the 24-hour time point.

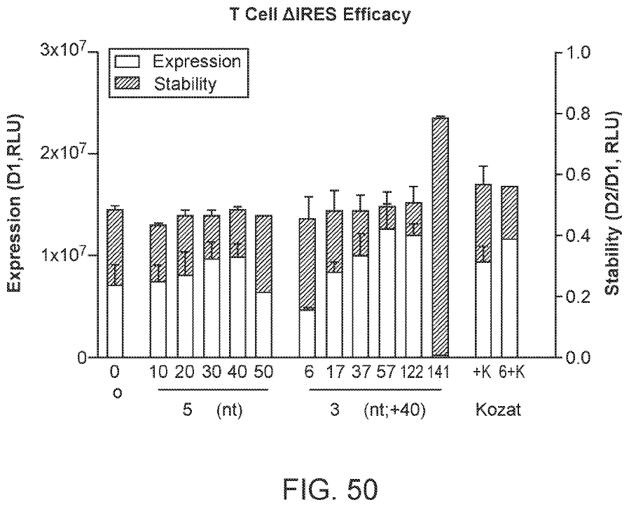

As shown in FIG. 50, deletion of more than 40 nucleotides from the 5′ end of the IRES reduced expression and disrupted IRES function. Stability of expression was relatively unaffected by the truncation of the IRES element but expression level was substantially reduced by deletion of 141 nucleotides from the 3′ end of the IRES, whereas deletion of 57 or 122 nucleotides from the 3′ end had a positive impact on the expression level.

It was also observed that deletion of the 6-nucleotide pre-start sequence reduced the expression level of the luciferase reporter. Replacement of the 6-nucleotide sequence with a classical kozak sequence (GCCACC) did not have a significant impact but at least maintained expression.

### Example 50

This example describes modifications (e.g., truncations) of selected IRES sequences, including Caprine Kobuvirus (CKV) IRES, Parabovirus IRES, Apodemus Picornavirus (AP) IRES, Kobuvirus SZAL6 IRES, Crohivirus B (CrVB) IRES, CVB3 IRES, and SAFV IRES. The sequences of the IRES elements are provided in SEQ ID NOs: 348-389. Briefly, circular RNA constructs were generated with truncated IRES elements operably linked to a Gaussia luciferase coding sequence. HepG2 cells were transfected with the circular RNAs. Luminescence in the supernatant was assessed 24 and 48 hours after transfection. Stability of expression was calculated as the ratio of the expression level at the 48-hour time point relative to that at the 24-hour time point.

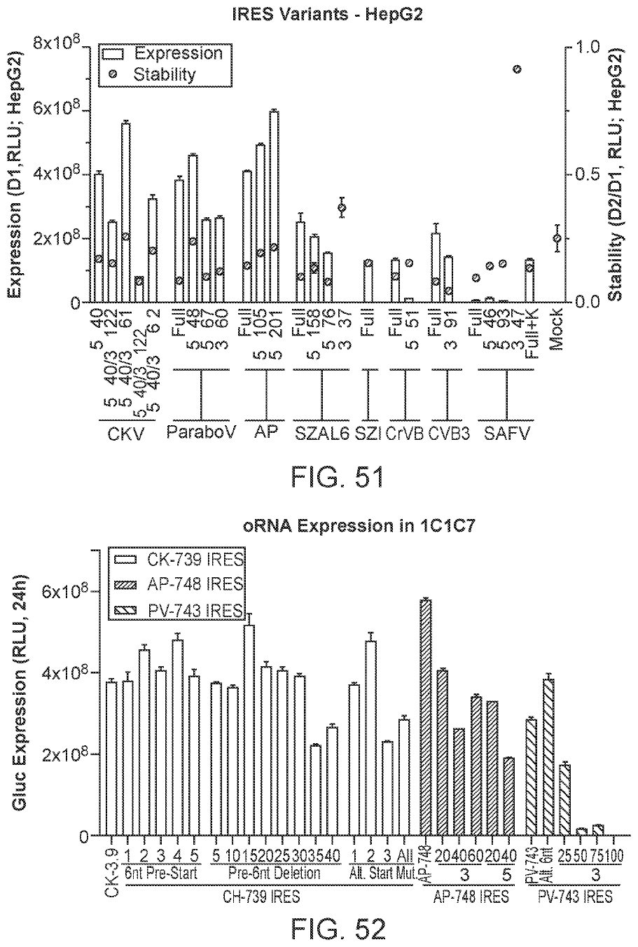

As shown in FIG. 51, truncations had variable effects depending on the identity of the IRES, which may depend on the initiation mechanism and protein factors used for translation, which often differs between IRESs. 5′ and 3′ deletions can be effectively combined, for example, in the context of CKV IRES. Addition of a canonical Kozak sequence in some cases significantly improved expression (as in SAFV, Full vs Full+K) or diminished expression (as in CKV, 5d40/3d122 vs 5d40/3d122+K).

### Example 51

This example describes modifications of CK-739, AP-748, and PV-743 IRES sequences, including mutations alternative translation initiation sites. Briefly, circular RNA constructs were generated with modified IRES elements operably linked to a Gaussia luciferase coding sequence. Luminescence from secreted Gaussia luciferase in supernatant was measured 24 and 48 hours after transfection of 1C1C7 cells with RNA.

CUG was the most commonly found alternative start site but many others were also characterized. These triplets can be present in the IRES scanning tract prior to the start codon and can affect translation of correct polypeptides. Four alternative start site mutations were created, with the IRES sequences provided in SEQ ID NOs: 378-380. As shown in FIG. 52, mutations of alternative translation initiation sites in the CK-739 IRES affected translation of correct polypeptides, positively in some instances and negatively in other instances. Mutation of all the alternative translation initiation sites reduced the level of translation.

Alternative Kozak sequences, 6 nucleotides before start codon, can also affect expression levels. The 6-nucleotide sequence upstream of the start codon were gTcacG, aaagtc, gTcacG, gtcatg, gcaaac, and acaacc, respectively, in CK-739 IRES and Sample Nos. 1-5 in the “6 nt Pre-Start” group. As shown in FIG. 52, substitution of certain 6-nucleotide sequences prior to the start codon affected translation.

It was also observed that 5′ and 3′ terminal deletions in AP-748 and PV-743 IRES sequences reduced expression. However, in the CK-739 IRES, which had a long scanning tract, translation was relatively unaffected by deletions in the scanning tract.

### Example 52

This example describes modifications of selected IRES sequences by inserting 5′ and/or 3′ untranslated regions (UTRs) and creating IRES hybrids. Briefly, circular RNA constructs were generated with modified IRES elements operably linked to a Gaussia luciferase coding sequence. Luminescence from secreted Gaussia luciferase in supernatant was measured 24 and 48 hours after transfection of HepG2 cells with RNA.

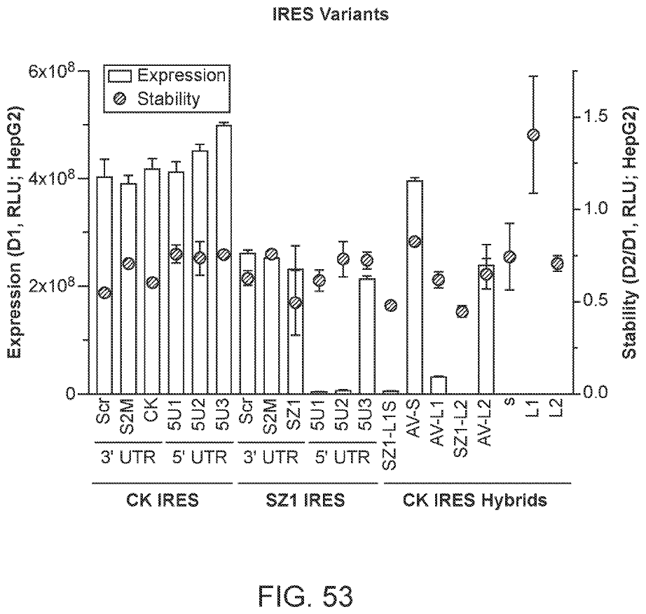

IRES sequences with UTRs inserted are provided in SEQ ID NOs: 390-401. As shown in FIG. 53, insertion of 5′ UTR right after the 3′ end of the IRES and before the start codon slightly increased the translation from Caprine Kobuvirus (CK) IRES but in some instances abrogated translation from Salivirus SZ1 IRES. Insertion of 3′ UTR right after the stop cassette had no impact on both IRES sequences.

Hybrid CK IRES sequences are provided in SEQ ID NOs: 390-401. CK IRES was used as a base, and specific regions of the CK IRES were replaced with similar-looking structures from other IRES sequences, for example, SZ1 and AV (Aichivirus). As shown in FIG. 53, certain hybrid synthetic IRES sequences were functional, indicating that hybrid IRES can be constructed using parts from distinct IRES sequences that show similar predicted structures while deleting these structures completely abrogates IRES function.

### Example 53

This example describes modifications of circular RNAs by introducing stop codon or cassette variants. Briefly, circular RNA constructs were generated with IRES elements operably linked to a Gaussia luciferase coding sequence followed by variable stop codon cassettes, which included a stop codon in each frame and two stop codons in the reading frame of the Gaussia luciferase coding sequence. 1C1C7 cells were transfected with the circular RNAs. Luminescence in supernatant was assessed 24 and 48 hours after transfection.

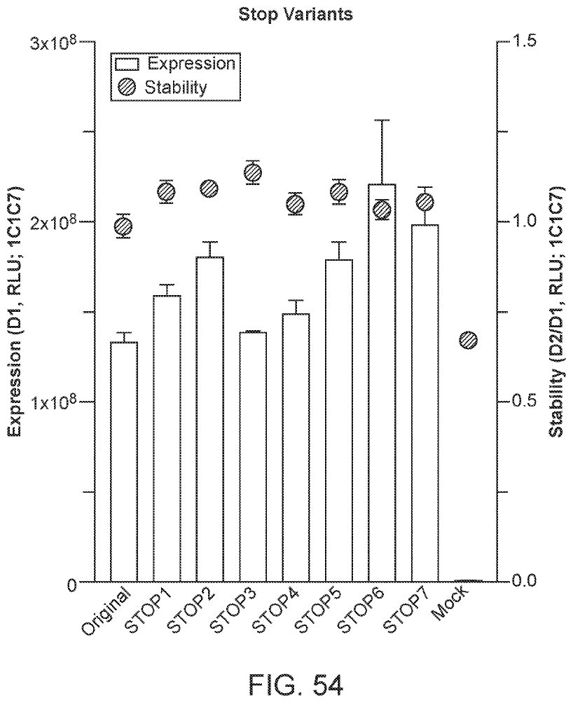

The sequences of the stop codon cassettes are set forth in SEQ ID NOs: 406-412. As shown in FIG. 54, certain stop codon cassettes improved expression levels, although they had little impact on expression stability. In particular, a stop cassette with two frame 1 (the reading frame of the Gaussia luciferase coding sequence) stop codons, the first being TAA, followed by a frame 2 stop codon and a frame 3 stop codon, is effective for promoting functional translation.

### Example 54

This example describes modifications of circular RNAs by inserting 5′ UTR variants. Briefly, circular RNA constructs were generated with IRES elements with 5′ UTR variants inserted between the 3′ end of the IRES and the start codon, the IRES being operably linked to a Gaussia luciferase coding sequence. 1C1C7 cells were transfected with the circular RNAs. Luminescence in supernatant was assessed 24 and 48 hours after transfection.

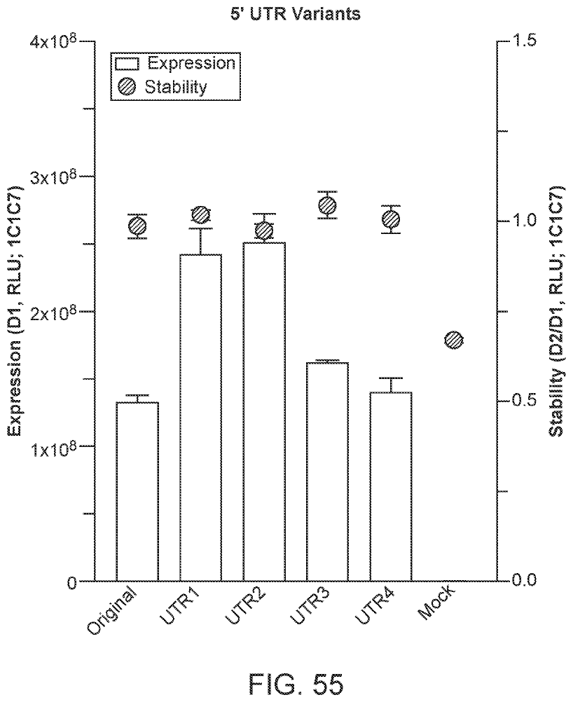

The sequences of the 5′ UTR variants are set forth in SEQ ID NOs: 402-405. As shown in FIG. 55, a CK IRES with a canonical Kozak sequence (UTR4) was more effective when a 36-nucleotide unstructured/low GC spacer sequence was added (UTR2), suggesting that the GC-rich Kozak sequences may interfere with core IRES folding. Using a higher-GC/structured spacer with a kozak sequence did not show the same benefit (UTR3), possibly due to interference with IRES folding by the spacer itself. Mutating the kozak sequence to gTcacG (UTR1) enhanced translation to the same level as the Kozak+spacer alternative without the need for a spacer.

### Example 55

This example describes the impact of miRNA target sites in circular RNAs on expression levels. Briefly, circular RNA constructs were generated with IRES elements operably linked to a human erythropoietin (hEPO) coding sequence, where 2 tandem miR-122 target sites were inserted into the construct. miR-122-expressing Huh7 cells were transfected with the circular RNAs. hEPO expression in supernatant was assessed 24 and 48 hours after transfection by sandwich ELISA.

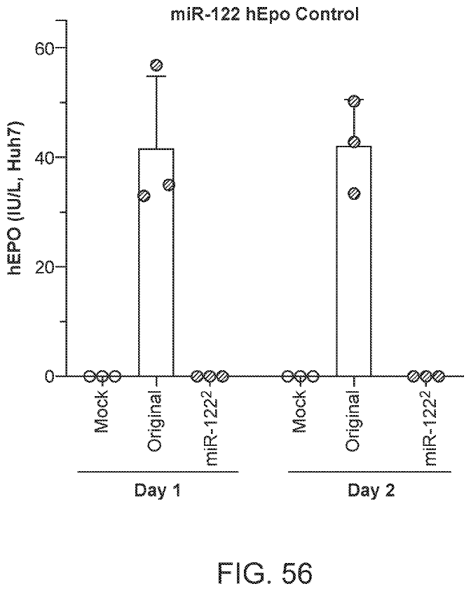

As shown in FIG. 56, the hEPO expression level was obrogated where the miR-122 target sites were inserted into the circular RNA. This result demonstrates that expression from circular RNA can be regulated by miRNA. As such, cell type- or tissue-specific expression can be achieved by incorporating target sites of the miRNAs expressed in the cell types in which expression of the recombinant protein is undesirable.

### Example 56

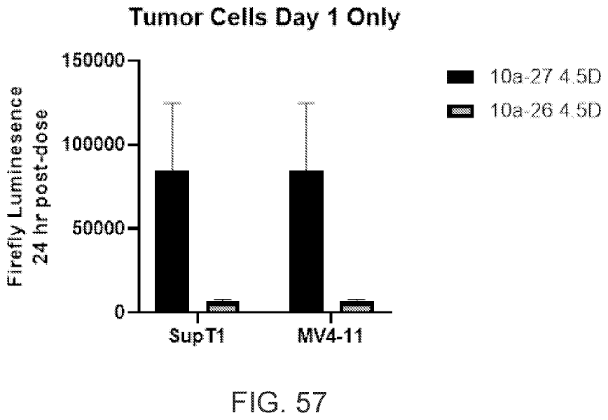

This example shows transfection of human tumor cells by LNPs in vitro. SupT1 cells (a human T cell tumor line) and MV4-11 cells (a human macrophage tumor line) were plated at 100,000 cells/well and 100,000 cells/well, respectively, in a 96-well plate overnight. Then, LNPs containing oRNA coding for Firefly Luciferase (FLuc) were added to the cells at 200 ng RNA/well. After 24-hour incubation, luminescence was quantified using the BrightGlo Luciferase Assay System (Promega) according to manufacturer's instructions and background luminescence from cells not treated with LNP was subtracted. FIG. 57 quantifies the measured Firefly luminescence, indicating that LNPs comprising Lipid 27 from Table 10a (10a-27 (4.5D) LNP, see Example 70) or Lipid 26 from Table 10a (10a-26 (4.5D) LNP, see Example 70) can transfect and express oRNA in both human T cell and macrophage tumor lines in vitro. 10a-27 (4.5D) LNP resulted in higher luminescence than 10a-26 (4.5D) LNP showing that levels of transfection of LNPs to human tumor cells can be affected by formulation.

### Example 57

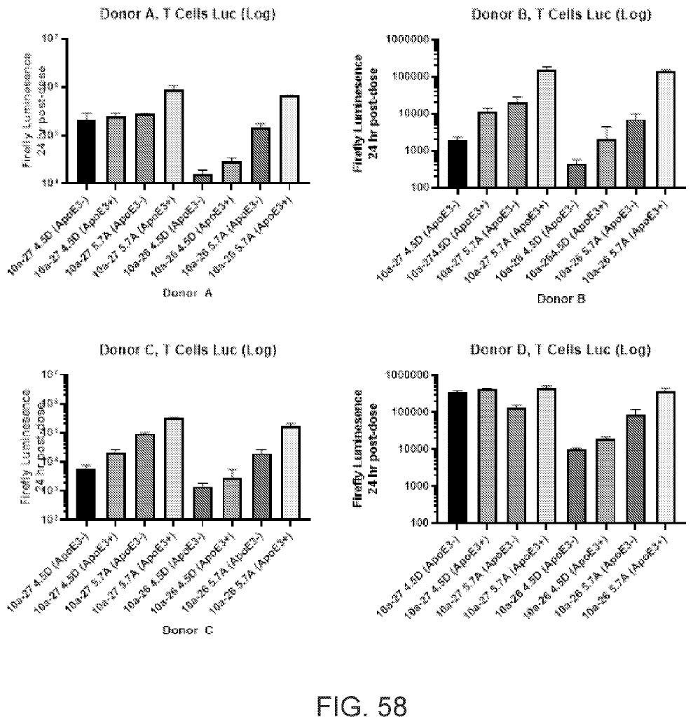

This example shows transfection of primary human activated T cells in vitro. Primary human T cells from independent donors were stimulated with aCD3/aCD28 and allowed to proliferate for 6 days in the presence of human serum and IL-2. Then, 100,000 cells were plated in a 96 well plate and LNPs containing oRNA coding for Firefly Luciferase (FLuc) were added to the cells at 200 ng RNA/well with or without Apolipoprotein E3 (ApoE3). After 24-hour incubation, luminescence was quantified using the Bright-Glo Luciferase Assay System (Promega) according to manufacturer's instructions and background luminescence from cells not treated with LNPs was subtracted. FIG. 58 shows the measured Firefly luminescence across 4 independent donors, demonstrating that all LNPs tested transfect primary human T cells in vitro. LNPs containing Lipid 27 from Table 10a (10a-27) generally produced higher luminescence than those containing Lipid 26 from Table 10a (10a-26). Furthermore, the addition of ApoE3 generally increased the expression of luciferase more for 10a-27 (5.7A) and 10a-26 (5.7A) (average of 4.4-fold and 9.3-fold across 4 donors, respectively) compared to 10a-27 (4.5D) and 10a-26 (4.5D) (3.1-fold and 2.6-fold, respectively). This suggests that the helper lipid, PEG lipid, and ionizable lipid:phosphate ratio all contribute to the ApoE-dependence of different formulations made with the same ionizable lipids. (See Example 70 for LNP formulation procedure, e.g., for 10a-27 (5.7A), 10a-26 (5.7A), 10a-27 (4.5D), and 10a-26 (4.5D) LNPs.)

### Example 58

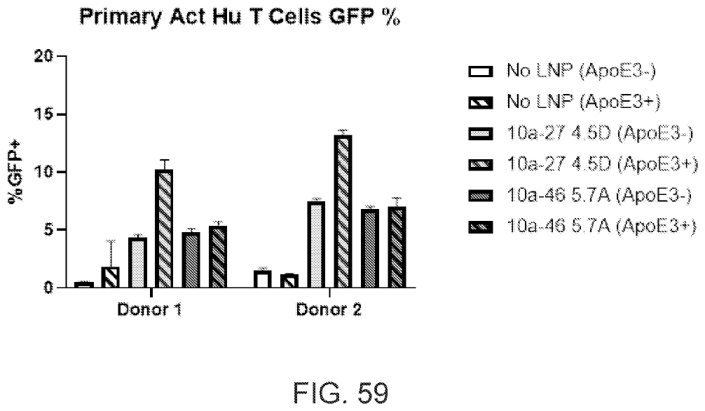

This example shows that different tail chemistries of LNPs result in different uptake mechanisms into T cells. To quantify the percent of human T cells expressing oRNA, LNPs containing eGFP oRNA were added to activated primary human T cells (prepared as described above in Example 57) at 200 ng RNA/well with or without ApoE3. After 24-hour incubation, cells were analyzed by flow cytometry and the percentage of live, GFP+ T cells was quantified. FIG. 59 graphs the % GFP+ T cells for 2 independent donors, with 5-10% of cells being GFP+ for LNP containing Lipid 27 from Table 10a (10a-27 (4.5D) LNP, see Example 70) and for LNP contacting Lipid 46 from Table 10a (10a-46 (5.7A) LNP, see Example 70). Although ApoE3 addition resulted in increased transfection for 10a-27 (4.5D) LNP, it did not appear to increase transfection for 10a-46 (5.7A) LNP, suggesting the different tail chemistries between Lipids 10a-27 and 10a-46 may mediate different uptake mechanisms into T cells.

### Example 59

This example describes immune cell expression of Cre in a Cre reporter mouse model.

Ai9 mice (B6.Cg-Gt(ROSA)26Sortm9(CAG-tdTomato)Hze/J, female, 6-8 weeks, n=3 per group) were injected i.v. with 0.5 mg/kg Cre oRNA LNPs or PBS. Ai9 mice transcribe and translate the fluorescent reporter tdTomato upon Cre recombination; meaning cells which are tdTomato+ have successfully been transfected with Cre oRNA. After 48 hours, mice were euthanized and their spleens were collected and manually processed into single cell suspensions. The splenocytes were stained for dead cells (LiveDead Fixable Aqua, Thermo) and with anti-mouse antibodies (TCR-B chain, BV421, H57-597; CD45, BV711, 30-F11; CD11b, BV785, ICRF44; NKp46, AF647, 29A1.4; CD19, APC/750, 6D5; TruStain FcX, 93; all antibodies from Biolegend) at 1:200 ratio. Flow cytometry was performed using an Attune Nxt Flow Cytometer (Thermo).

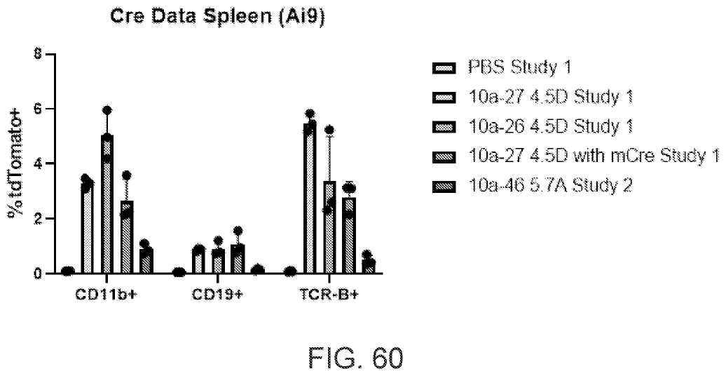

The percent of tdTomato+ cells in splenic myeloid cells (CD11b+), B cells (CD19+), and T cells (TCR-B+) is presented in FIG. 60. Lipid 10a-27 and Lipid 10a-46 differ only by their tail chemistries, and formulations made with Lipid 10a-27 transfect significantly more splenic immune cells than those made with Lipid 10a-46. Additionally, 10a-27 (4.5D) LNP (see Example 70) formulated with Cre oRNA transfected approximately twice as many T cells than those formulated with Cre linear mRNA, suggesting that oRNA may result in improved protein expression in splenic T cells compared to linear mRNA.

TABLE 37
Characterization of LNPs

| | Z-Average | | RNA Encapsulation |
|---|---|---|---|
| Formulation | (nm) | PDI | Efficiency (%) |
| 10a-27 (4.5D), Study 1 | 65 | 0.07 | 96 |
| 10a-26 (4.5D), Study 1 | 74 | 0.06 | 94 |
| 10a-27 (4.5D), with mCre, | 75 | 0.05 | 93 |
| Study 1 | | | |
| 10a-46 (5.7A), Study 2 | 86 | 0.01 | 94 |

### Example 60

This example shows immune cell expression of mOX40L oRNA in wildtype mice.

C57BL/6 mice (female, 6-8 weeks, n=3 or 4 per group) were injected intravenously with 0.5 mg/kg mOX40L oRNA LNPs or PBS. After 24 hours, mice were euthanized and their spleens were collected and manually processed into single cell suspensions. Splenocytes were stained for dead cells (LiveDead Fixable Aqua, Thermo) and with anti-mouse antibodies (TCR-B chain, PacBlue, H57-597; CD11b, FITC, ICRF44; B220, PE, RA3-6B2; CD45, PerCP, 30-F11; mOX40L, AF647, RM134L; NK1.1, APC/750, PK136; TruStain FcX, 93; all antibodies from Biolegend) at 1:200. Flow cytometry was performed using an Attune NxT Flow Cytometer (Thermo).

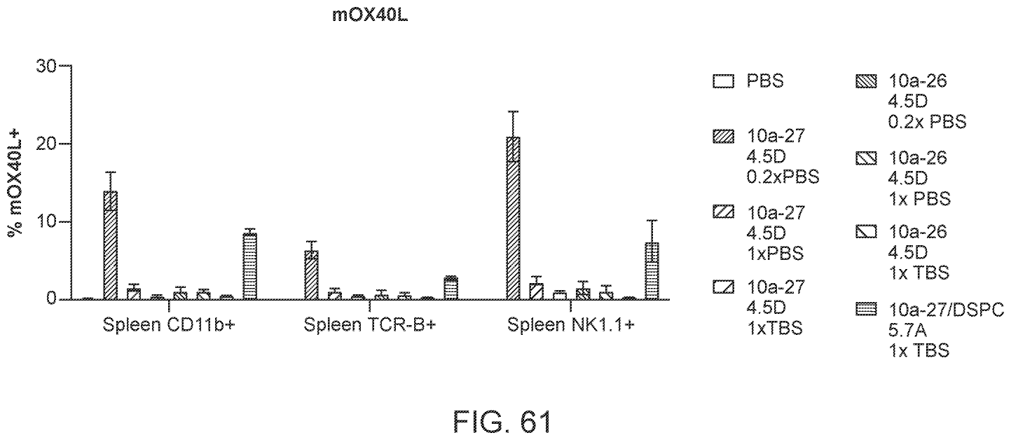

The percent of mOX40L+ cells in splenic myeloid (CD11b+), T cells (TCR-B+), and NK cells (NK1.1+) is presented in FIG. 61. Notably, significantly different transfection efficiencies are observed between the same formulations injected intravenously in different buffers (hypotonic PBS, isotonic PBS, and isotonic TBS). 10a-27 4.5D LNP in hypotonic PBS results in approximately 14% myeloid cell transfection, 6% T cell transfection, and 21% NK cell transfection in the spleen. Of the formulations injected in isotonic buffer, 10a-27 DSPC 5.7A LNP demonstrates myeloid, T cell, and NK cell transfection in the spleen (9%, 3%, and 8%, respectively). (See Example 70 for LNP formulation procedure, e.g., for 10a-27 (4.5D) LNP and 10a-27 DSPC (5.7A) LNP.)

TABLE 38
Characterization of LNPs

| | Z-Average | | RNA Encapsulation |
|---|---|---|---|
| Formulation | (nm) | PDI | Efficiency (%) |
| 10a-27 (4.5D) | 63 | 0.02 | 93 |
| 10a-26 (4.5D) | 67 | 0.07 | 94 |
| 10a-27 DSPC (5.7A) | 82 | 0.05 | 96 |

### Example 61

This example shows single dose escalation of mOX40L oRNA-LNPs in wildtype mice.

57BL/6 mice (female, 6-8 weeks, n=3 per group) were injected intravenously with 1 mg/kg or 3 mg/kg mOX40L oRNA LNPs or buffer control. After 24 hours, mice were euthanized and their spleens were collected and manually processed into single cell suspensions. Splenocytes were stained for dead cells (LiveDead Fixable Blue, Thermo) and stained with anti-mouse antibodies (TCR-B chain, BV421, H57-597; CD19, BV605, 6D5; CD45, BV711, 30-F11; CD11b, BV785, ICRF44; CD11c, FITC, N418; CD8a, PerCP, 53-6.7; mOX40L, PE, RM134L; NKp46, AF647, 29A1.4; CD4, APC/750, GK1.5; TruStain FcX, 93; all antibodies from Biolegend) at 1:200. Flow cytometry was performed using a BD FACS Symphony flow cytometer.

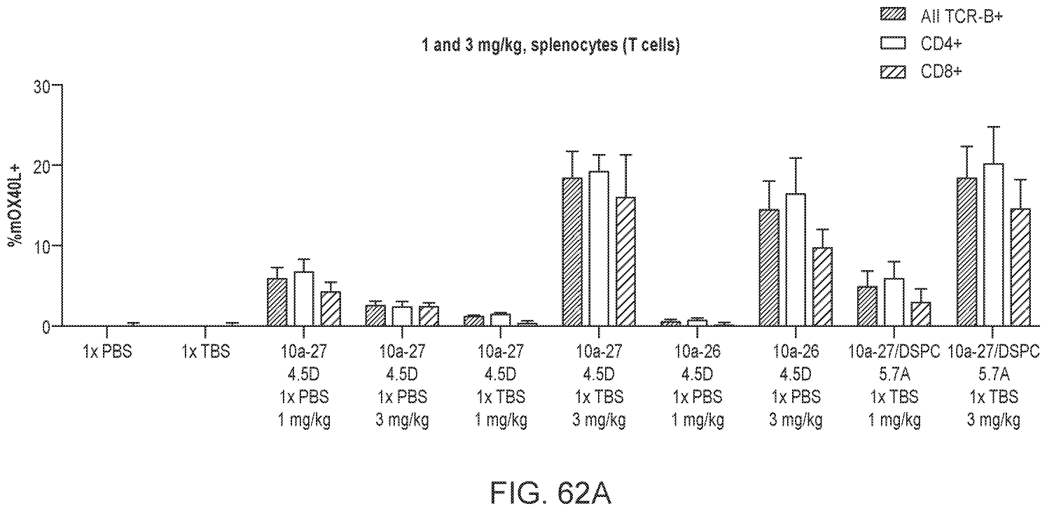

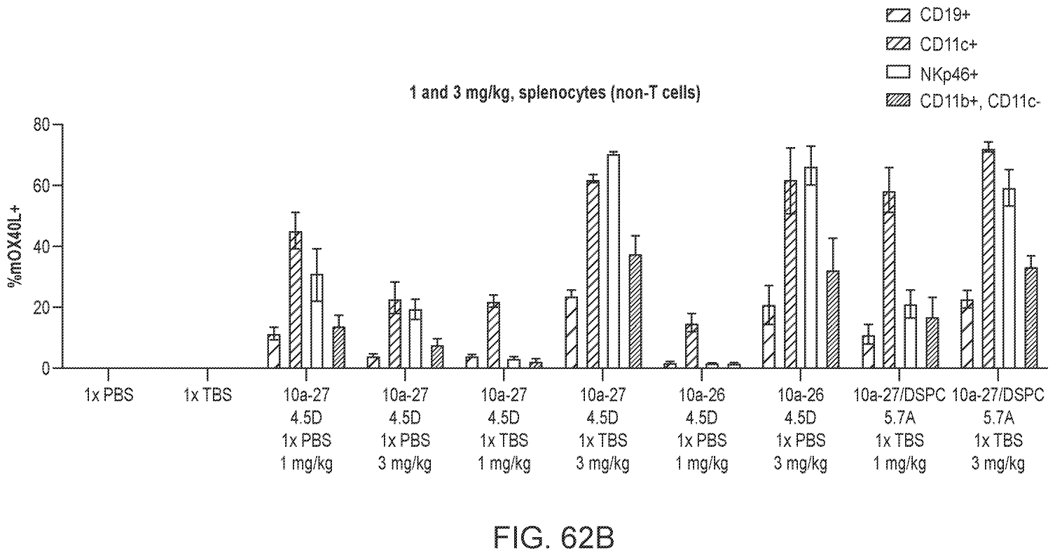

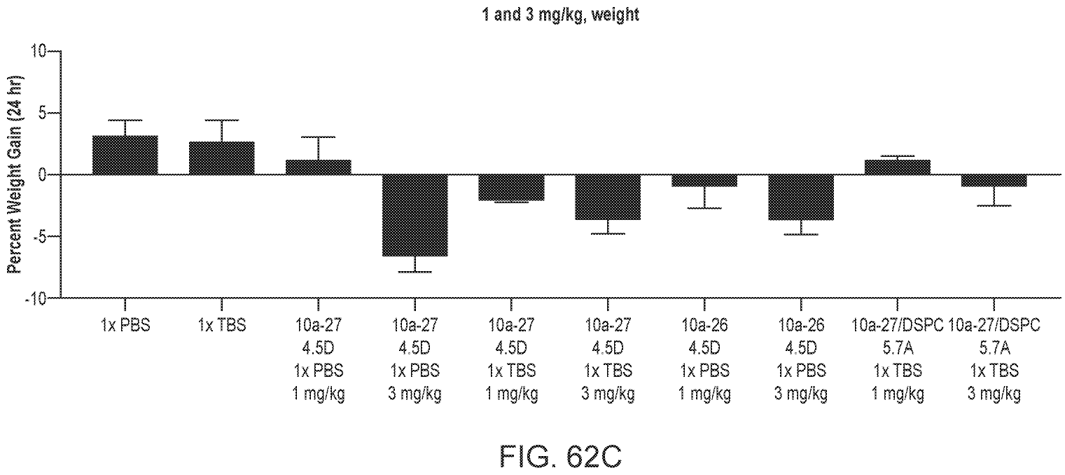

The percent of mOX40L+ cells in splenic T cells (all TCR-B+), CD4+ T cells (TCR-B+, CD4+), CD8+ T cells (TCR-B+, CD8a+), B cells (CD19+), NK cells (NKp46+), dendritic cells (CD11c+), and other myeloid cells (CD11b+, CD11c−) are shown in FIG. 62A and FIG. 62B, with corresponding mouse weight change after 24 hours shown in FIG. 62C. A dose-dependent increase in immune cell subset transfection is observed across 1 mg/kg and 3 mg/kg for all groups, with the exception of 10a-27 (4.5D) LNP 1×PBS group. At the 3 mg/kg dose, three different LNPs (10a-27 (4.5D) in TBS, 10a-26 (4.5D) in PBS, and 10a-27 DSPC (5.7A) in TBS; see Example 70 for formulation procedures) achieve 10-20% mOX40L transfection in splenic T cells, with similar transfection rates observed among CD4+ and CD8+ subsets. These three formulations also result in approximately 20% B cell, 60-70% dendritic cell, 60-70% NK cell, and 30-40% other myeloid cell mOX40L transfection in the spleen at 3 mg/kg. These three formulations lead to only minor (0-3%) mouse weight loss at 24 hours at the 3 mg/kg single dose with no reported clinical observations.

TABLE 39
Characterization of LNPs

| | Z-Average | | RNA Encapsulation |
|---|---|---|---|
| Formulation | (nm) | PDI | Efficiency (%) |
| 10a-27 (4.5D) | 76 | 0.06 | 91 |
| 10a-26 (4.5D) | 67 | 0.01 | 88 |
| 10a-27 DSPC (5.7A) | 77 | 0.01 | 93 |

### Example 62

This example shows oRNA-LNP CAR-mediated B cell depletion in mice.

C57BL/6 mice (female, 6-8 weeks, n=5 per group) were injected intravenously with 0.5 mg/kg aCD19-CAR oRNA LNPs or control FLuc oRNA LNPs on Days 0, 2, 5, 7, and 9. On Days −1, 1, 8 and 12, submandibular bleeds were performed to collect blood. 30 uL of blood was lysed with ACK lysis buffer and washed with MACS buffer to isolate immune cells. On Day 12, mice were euthanized and their spleens were collected and manually processed into single cell suspensions. To assess the frequency of B cells in the blood and spleen, these single cell suspensions were stained for dead cells (LiveDead Fixable Aqua, Thermo) and stained with anti-mouse antibodies (TCR-B chain, PacBlue, H57-597; CD11b, FITC, ICRF44; B220, PE, RA3-6B2; CD45, PerCP, 30-F11; TruStain FcX, 93; all antibodies from Biolegend) at 1:200. Flow cytometry was performed using an Attune NxT Flow Cytometer (Thermo).

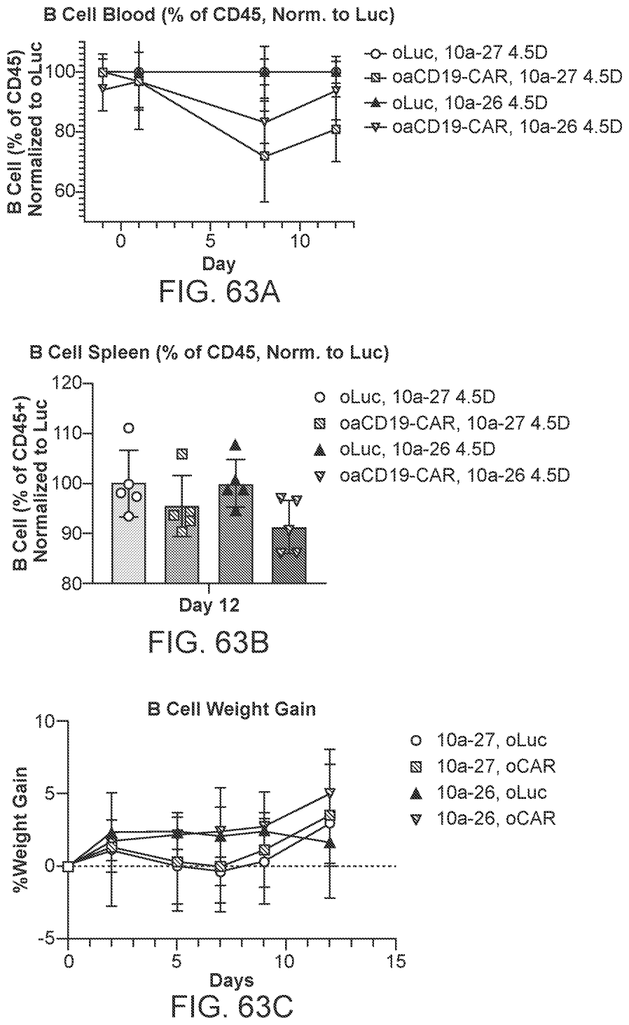

FIG. 63A quantifies the B cell depletion observed in this study, as defined by percentage of B220+ B cells of live, CD45+ immune cells. The B cell depletion in the aCD19-CAR oRNA LNP group was compared to its respective FLuc oRNA LNP control on Days 8 and 12 (for blood) and Day 12 (for spleen). In the blood, aCD19-CAR 10a-27 (4.5D) and 10a-26 (4.5D) LNPs resulted in approximately 28% and 17% reductions, respectively, in % B220+ of live CD45+ at Day 8 compared to FLuc control. In the spleen, aCD19-CAR 10a-27 (4.5D) and 10a-26 (4.5D) LNPs resulted in approximately 5% and 9% reductions in % B220+ of live CD45+ at Day 12 compared to FLuc control as shown in FIG. 63B. In all, these results suggest that CAR-mediated B cell depletion is occurring in mice treated with aCD19-CAR oRNA LNPs for Lipid 10a-27 (4.5D) and Lipid 10a-26 (4.5D).

In addition, FIG. 63C shows the percent weight gain of mice in this study. There was not significant weight loss on average from the 10a-27 4.5D or 10a-26 4.5D LNP treated mice (5×0.5 mg/kg over 9 days), suggesting that these LNPs may be well-tolerated in mice at this dose and schedule.

TABLE 40
Characterization of LNPs

| | Z-Average | | RNA Encapsulation |
|---|---|---|---|
| Formulation | (nm) | PDI | Efficiency (%) |
| 10a-27 (4.5D), oLuc | 65 | 0.03 | 93 |
| 10a-27 (4.5D), oaCD19-CAR | 74 | 0.02 | 96 |
| 10a-26 (4.5D), oLuc | 71 | 0.04 | 91 |
| 10a-26, (4.5D), oaCD19-CAR | 71 | 0.04 | 93 |

### Example 63

### LNP and Circular RNA Construct Containing Anti-CD 19 CAR Reduces B Cells in the Blood and Spleen In Vivo.

Circular RNA constructs encoding an anti-CD19 CAR expression were encapsulated within lipid nanoparticles as described above. For comparison, circular RNAs encoding luciferase expression were encapsulated within separate lipid nanoparticle.

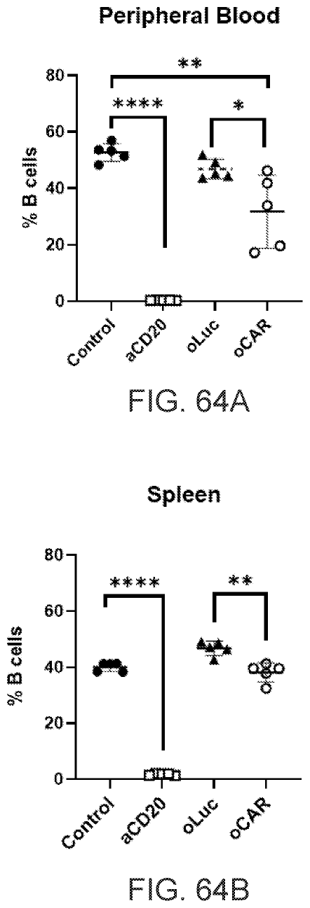

C57BL/6 mice at 6 to 8 weeks old were injected with either LNP solution every other day for a total of 4 LNP injections within each mouse. 24 hours after the last LNP injection, the mice's spleen and blood were harvested, stained, and analyzed via flow cytometry. As shown in FIG. 64A and FIG. 64B, mice containing LNP-circular RNA constructs encoding an anti-CD19 CAR led to a statistically significant reduction in CD19+ B cells in the peripheral blood and spleen compared to mice treated with LNP-circular RNA encoding a luciferase.

### Example 64

IRES Sequences Contained within Circular RNA Encoding CARs Improves CAR Expressions and Cytotoxicity of T-Cells.

Activated murine T-cells were electroporated with 200 ng of circular RNA constructs containing a unique IRES and a murine anti-CD19 1D3ζ CAR expression sequence. The IRES contained in these constructs were derived either in whole or in part from a Caprine Kobuvirus, Apodemus Picornavirus, Parabovirus, or Salivirus. A Caprine Kobuvirus derived IRES was additionally codon optimized. As a control, a circular RNA containing a wild-type zeta mouse CAR with no IRES was used for comparison. The T-cells were stained for the CD-19 CAR 24 hours post electroporation to evaluate for surface expression and then co-cultured with A20 Fluc target cells. The assay was then evaluated for cytotoxic killing of the Fluc+ A20 cells 24 hours after co-culture of the T-cells with the target cells.

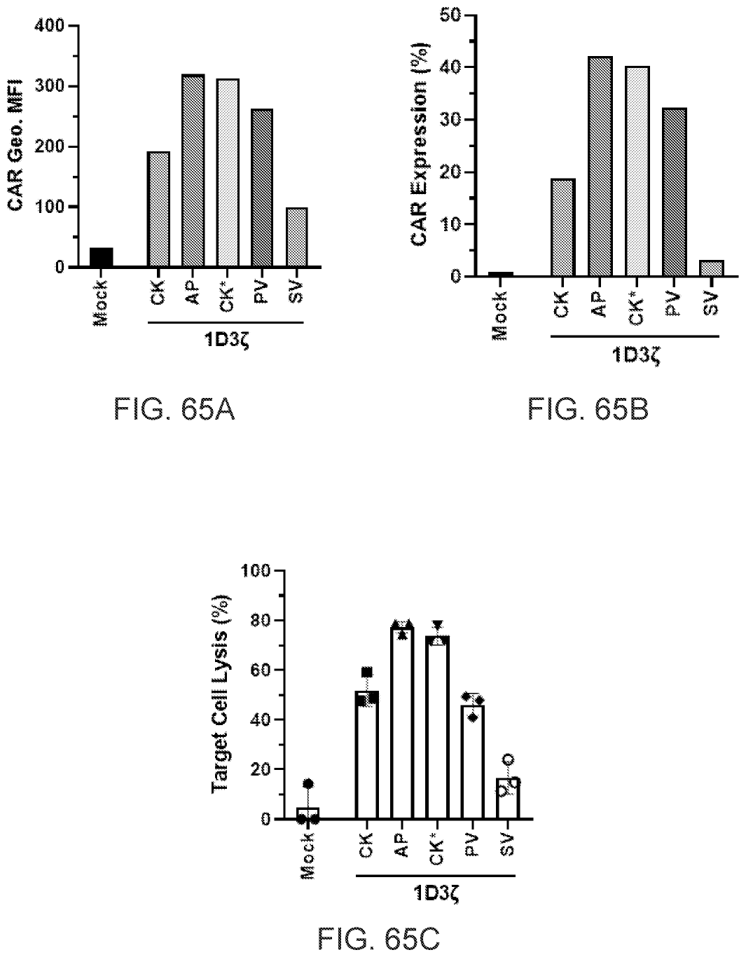

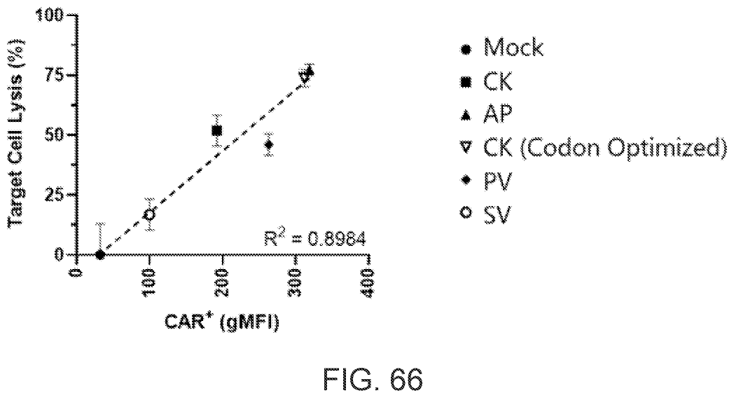

As seen in FIGS. 65A, 65B, 65C, and 66, the unique IRES were able to increase the frequency that the T-cells expressed the CAR protein and level of CAR expression on the surface of the cells. The increase frequency of expression of the CAR protein and level of CAR expression on the surface of cells lead to an improved anti-tumor response.

### Example 65

Cytosolic and Surface Proteins Expressed from Circular RNA Construct in Primary Human T-Cells.

Circular RNA construct contained either a sequence encoding for a fluorescent cytosolic reporter or a surface antigen reporter. Fluorescent reporters included green fluorescent protein, mCitrine, mWasabi, Tsapphire. Surface reporters included CD52 and Thy1.1^{bio}. Primary human T-cells were activated with an anti-CD3/anti-CD28 antibody and electroporated 6 days post activation of the circular RNA containing a reporter sequence. T-cells were harvested and analyzed via flow cytometry 24 hours post electroporation. Surface antigens were stained with commercially available antibodies (e.g., Biolegend, Miltenyi, and BD).

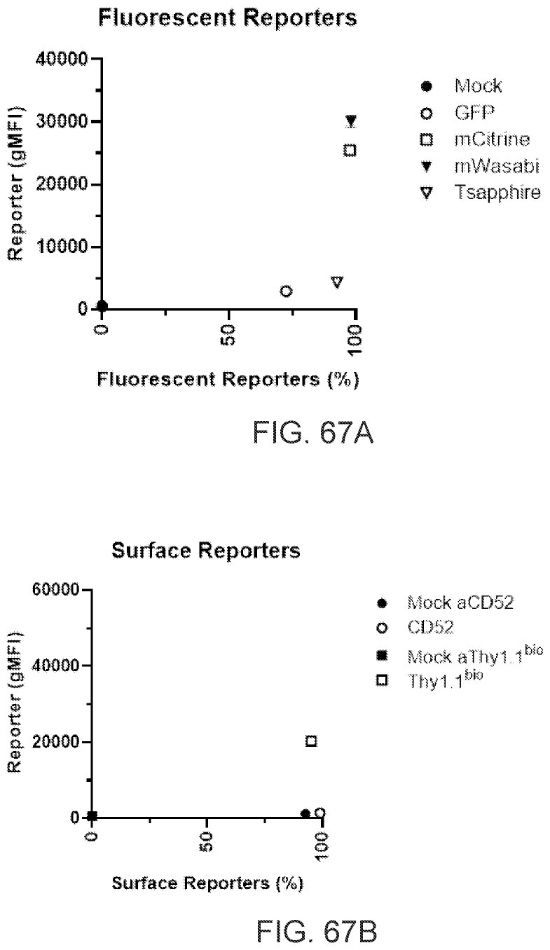

As seen in FIG. 67A and FIG. 67B, cytosolic and surface proteins can be expressed from circular RNA encoding the proteins in primary human T-cells.

### Example 66

Circular RNAs Containing Unique IRES Sequences have Improved Translation Expression Over Linear mRNA.

Circular RNA constructs contained a unique IRES along with an expression sequence for Firefly luciferase (FLuc).

Human T-cells from 2 donors were enriched and stimulated with anti-CD3/anti-CD28 antibodies. After several days of proliferation, activated T cells were harvested and electroporated with equal molar of either mRNA or circular RNA expressing FLuc payloads. Various IRES sequences, including those derived from Caprine Kobuvirus, Apodemus Picornavirus, and Parabovirus, were studied to evaluate expression level and durability of the payload expression across 7 days. Across the 7 days, the T-cells were lysed with Promega Brightglo to evaluate for bioluminsences.

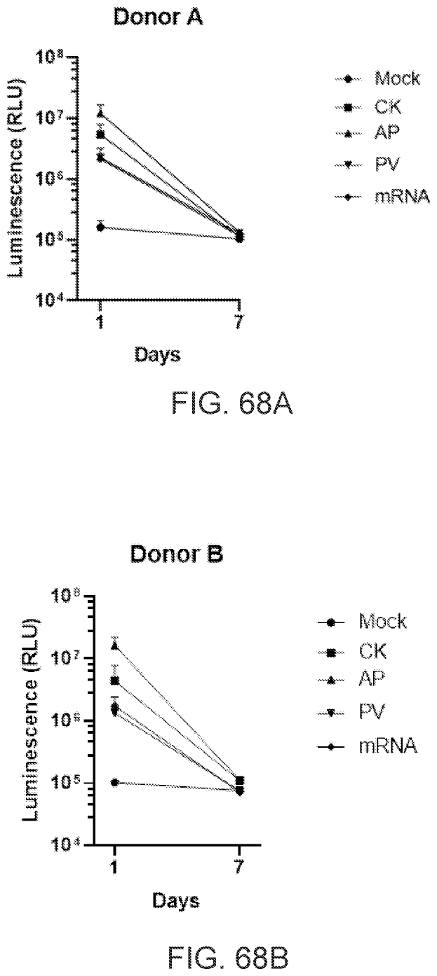

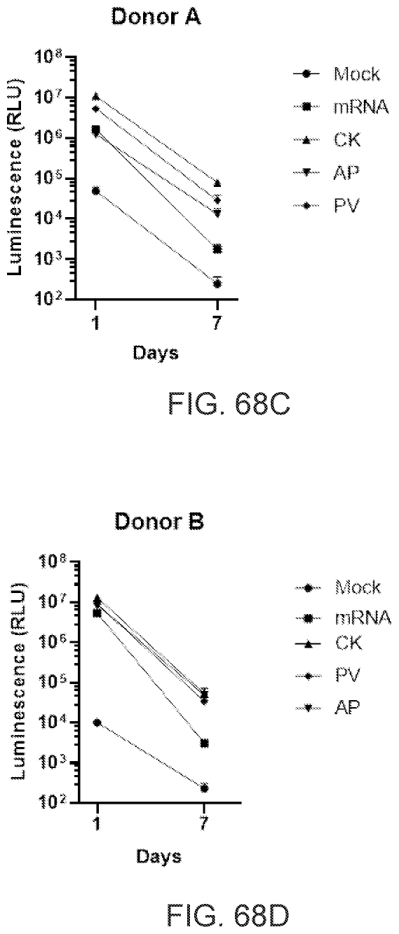

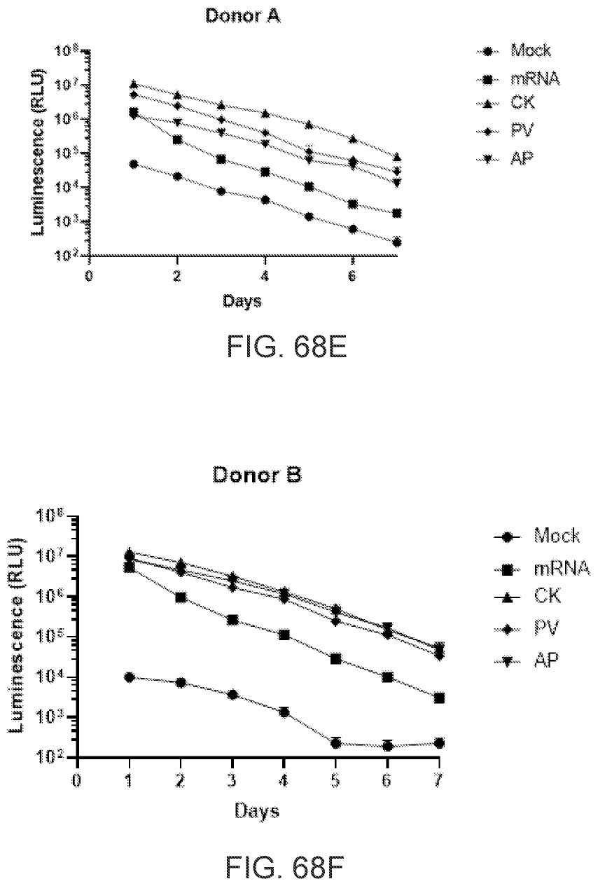

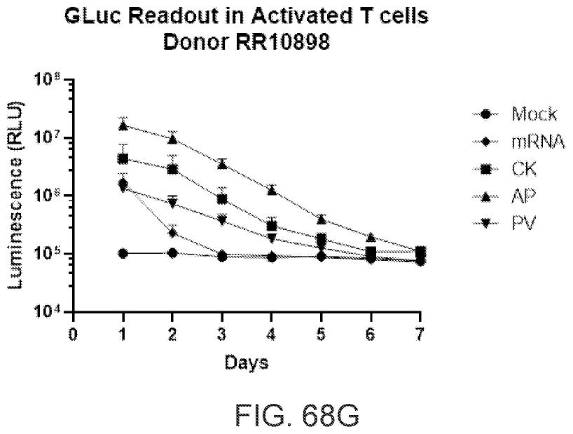

As shown in FIGS. 68C, 68D, 68E, 68F, and 68G, the presence of an IRES within a circular RNA can increase translation and expression of a cytosolic protein by orders of magnitude and can improve expression compared to linear mRNA. This was found consistent across multiple human T-cell donors.

### Example 67

### Example 65A: LNP-Circular RNA Encoding Anti-CD19 Mediates Human T-Cell Killing of K562 Cells

Circular RNA constructs contained a sequence encoding for anti-CD19 antibodies. Circular RNA constructs were then encapsulated within a lipid nanoparticle (LNP).

Human T-cells were stimulated with anti-CD3/anti-CD28 and left to proliferate up to 6 says. At day 6, LNP-circular RNA and ApoE3 (1 μg/mL) were co-cultured with the T-cells to mediate transfection. 24 hours later, Fluc+K562 cells were electroporated with 200 ng of circular RNA encoding anti-CD19 antibodies and were later co-cultured at day 7. 48 hours post co-culture, the assay was assessed for CAR expression and cytotoxic killing of K562 cells through Fluc expression.

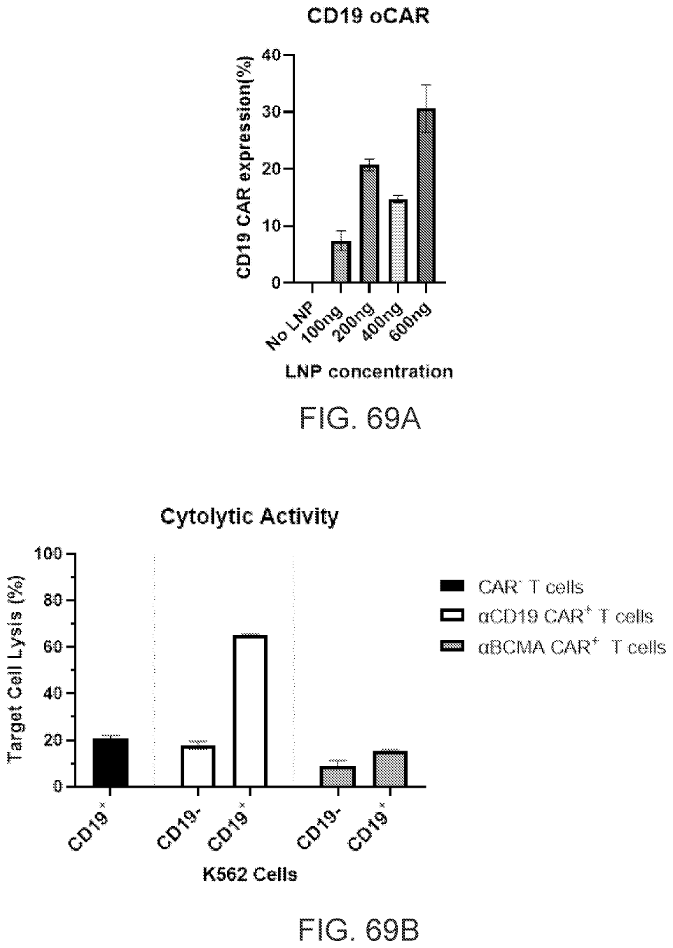

As shown in FIG. 69A and FIG. 69B, there is T-cell expression of anti-CD19 CAR from the LNP-mediated delivery of a CAR in vitro to T-cells and its capability to lyse tumor cells in a specific, antigen dependent manner in engineered K562 cells.

### Example 65B: LNP-Circular RNA Encoding Anti-BCMA Antibody Mediates Human T-Cell Killing of K562 Cells

Circular RNA constructs contained a sequence encoding for anti-BCMA antibodies. Circular RNA constructs were then encapsulated within a lipid nanoparticle (LNP).

Human T-cells were stimulated with anti-CD3/anti-CD28 and left to proliferate up to 6 says. At day 6, LNP-circular RNA and ApoE3 (1 μg/mL) were co-cultured with the T-cells to mediate transfection. 24 hours later, Fluc+K562 cells were electroporated with 200 ng of circular RNA encoding anti-BCMA antibodies and were later co-cultured at day 7. 48 hours post co-culture, the assay was assessed for CAR expression and cytotoxic killing of K562 cells through Fluc expression.

As shown in FIG. 69B, there is T-cell expression of BCMA CAR from the LNP-mediated delivery of a CAR in vitro to T-cells and its capability to lyse tumor cells in a specific, antigen dependent manner in engineered K562 cells.

### Example 68

### Anti-CD19 CAR T-Cells Exhibit Anti-Tumor Activity In Vitro.

Human T-cells were activated with anti-CD3/anti-CD28 and electroporated once with 200 ng of anti-CD19 CAR-expressing circular RNA. Electroporated T-cells were co-cultured with FLuc+ Nalm6 target cells and non-target Fluc+K562 cells to evaluate CAR-mediated killing. After 24 hours post co-culture, the T-cells were lysed and examined for remanent FLuc expression by target and non-target cells to evaluate expression and stability of expression across 8 days total.

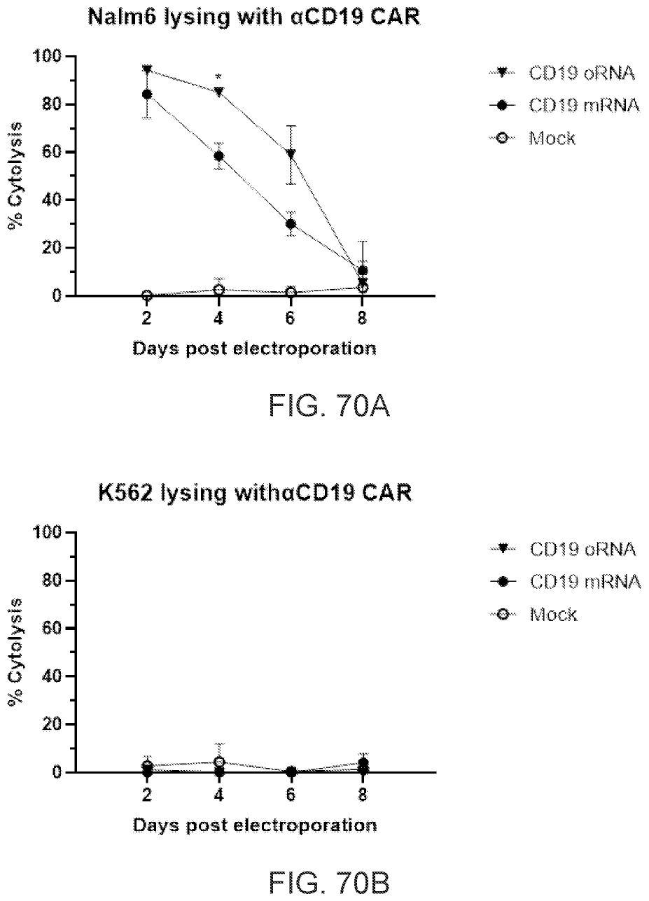

As shown in FIGS. 70A and 70B, T-cells express circular RNA CAR constructs in specific, antigen-dependent manner. Results also shows improved cytotoxicity of circular RNAs encoding CARs compared to linear mRNA encoding CARs and delivery of a functional surface receptor.

### Example 69

Effective LNP Transfection of Circular RNA Mediated with ApoE3

Human T-cells were stimulated with anti-CD3/anti-CD28 and left to proliferate up to 6 days. At day 6, lipid nanoparticle (LNP) was and circular RNA expressing green fluorescence protein solution with or without ApoE3 (1 μg/mL) were co-cultured with the T-cells. 24 hours later, the T-cells were stained for live/dead T-cells and the live T-cells were analyzed for GFP expression on a flow cytometer.

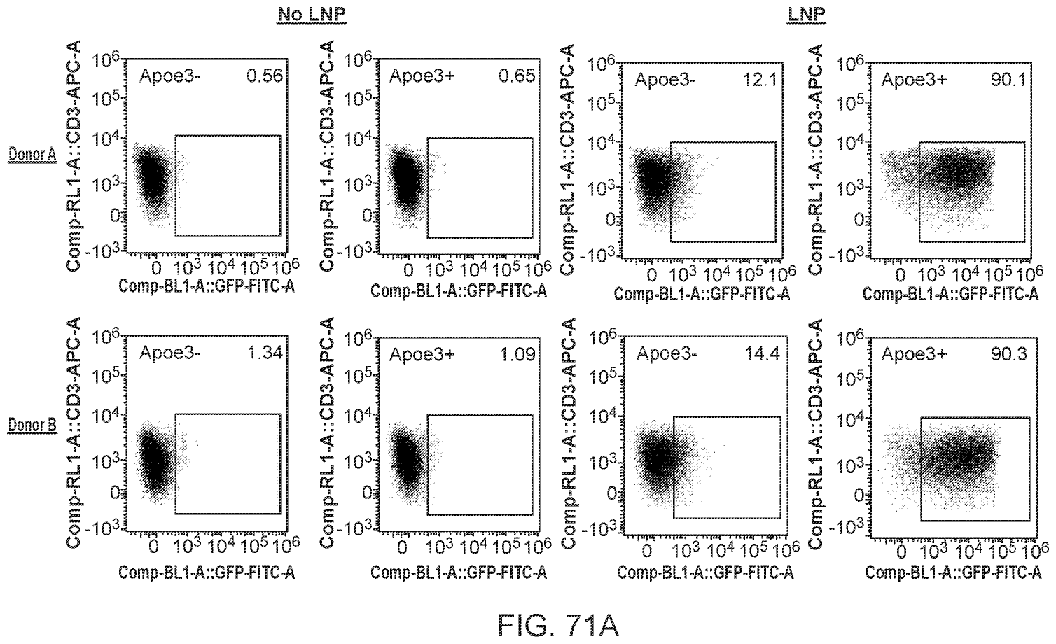

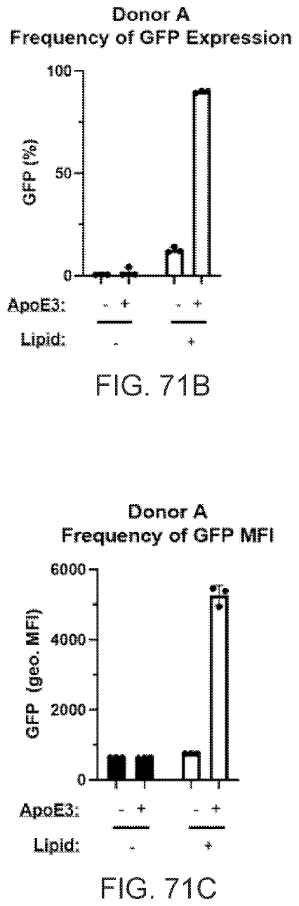

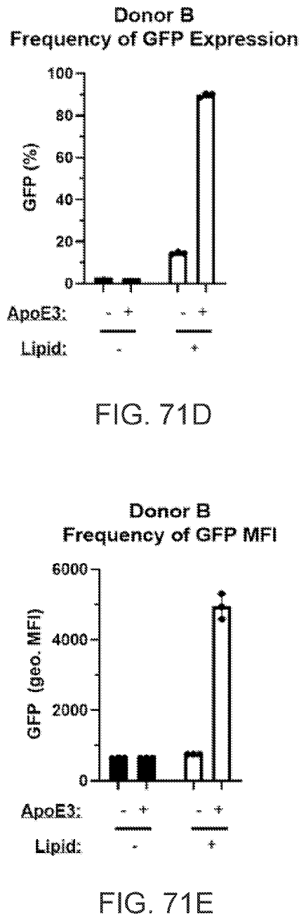

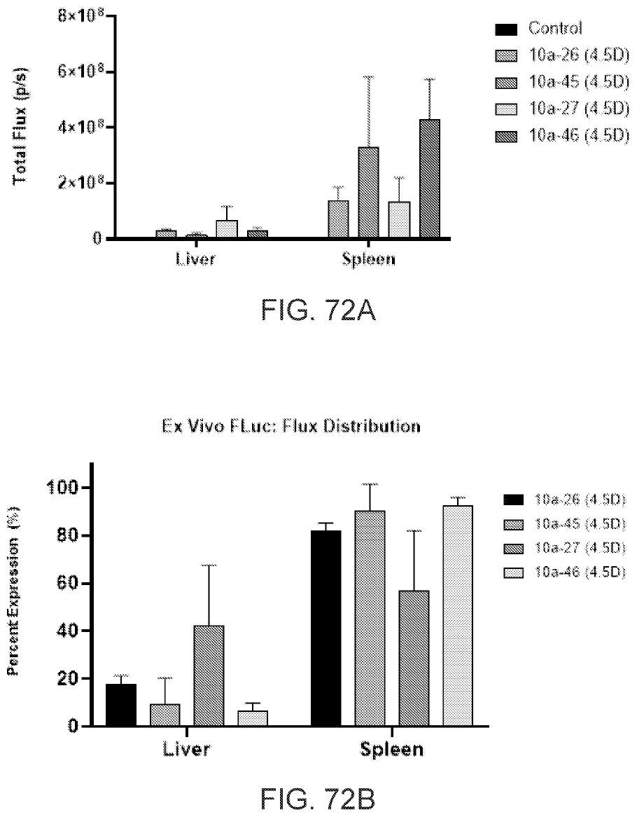

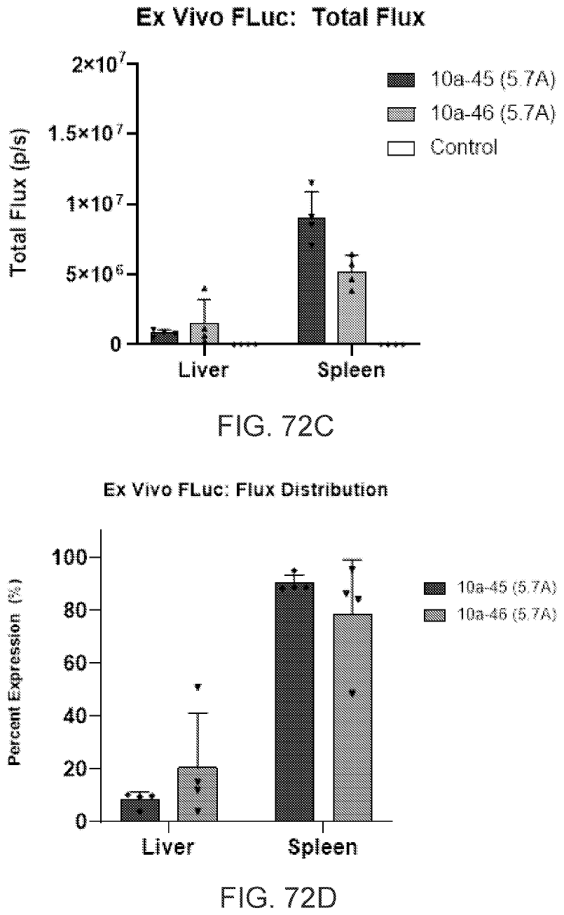

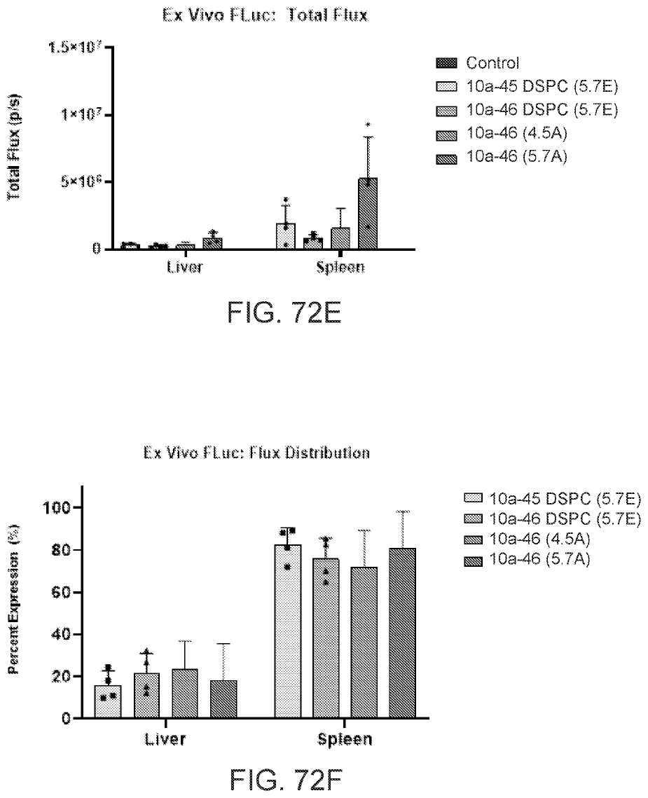

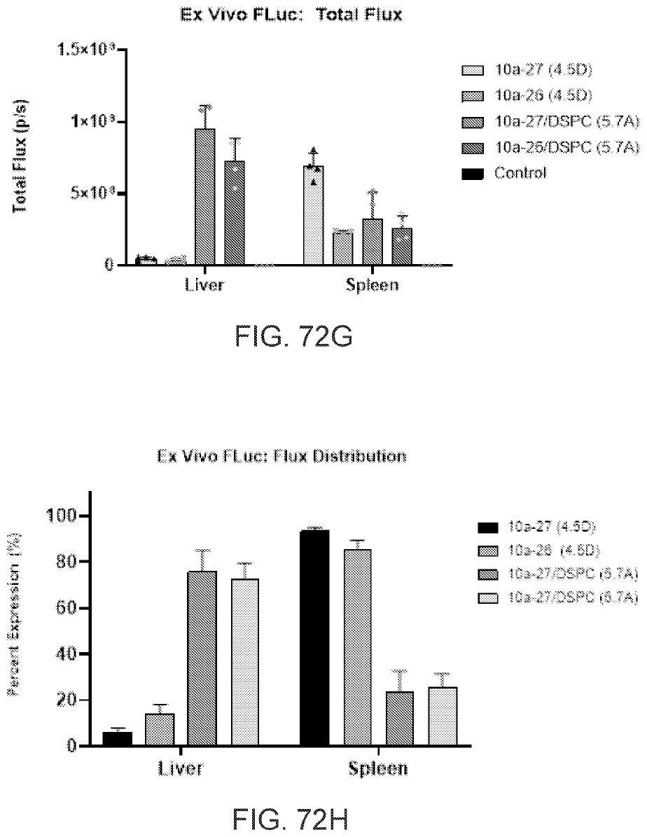

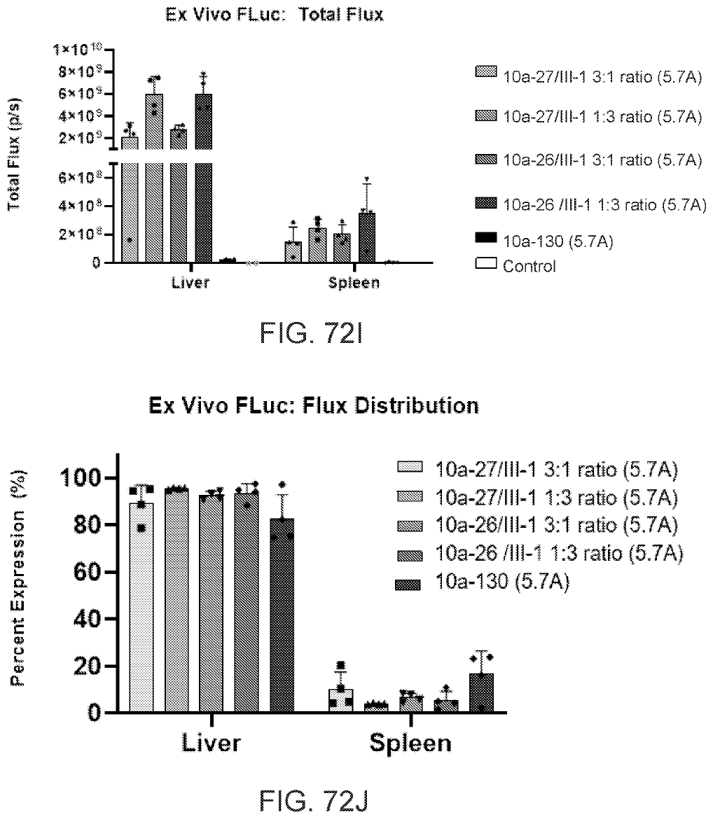

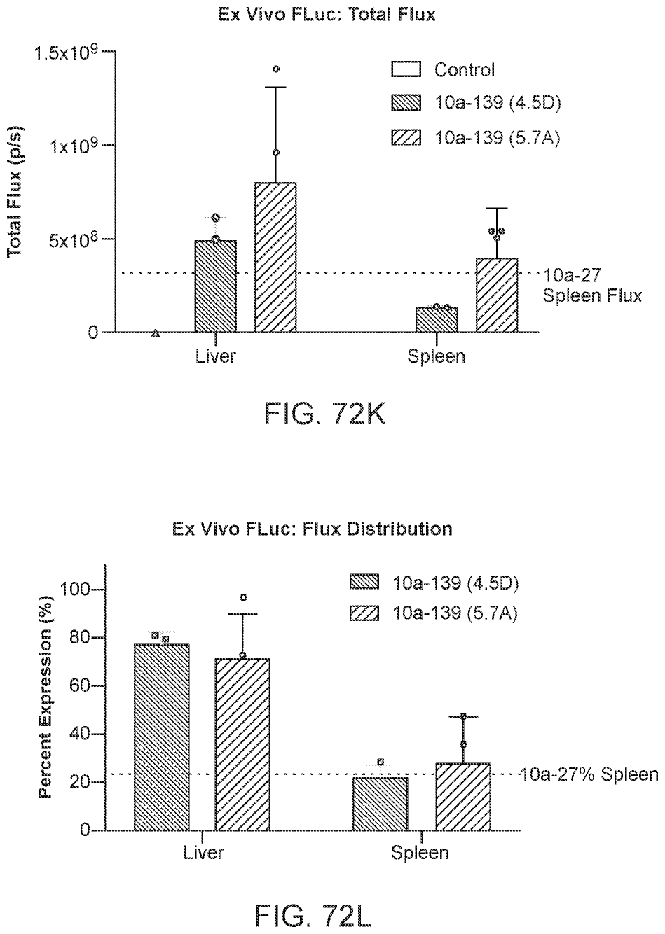

As shown by FIGS. 71A, 71B, 71C, 72D, and 71E, efficient LNP transfection can be mediated by ApoE3 on activated T-cells, followed by significant payload expression. These results were exhibited across multiple donors.

### Example 70

### Example 70A: Lipid Nanoparticle Formulation Procedure

A Zetasizer Nano ZS (Malvern Instruments Ltd, Malvern, Worcestershire, UK) can be used to determine the particle size, the polydispersity index (PDI) and the zeta potential of the transfer vehicle compositions in 1×PBS in determining particle size and 15 mM PBS in determining zeta potential.

Ultraviolet-visible spectroscopy can be used to determine the concentration of a therapeutic and/or prophylactic (e.g., RNA) in transfer vehicle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, Calif.). The concentration of therapeutic and/or prophylactic in the transfer vehicle composition can be calculated based on the extinction coefficient of the therapeutic and/or prophylactic used in the composition and on the difference between the absorbance at a wavelength of, for example, 260 nm and the baseline value at a wavelength of, for example, 330 nm.

For transfer vehicle compositions including RNA, a QUANT-IT™ RIBOGREEN® RNA assay (Invitrogen Corporation Carlsbad, Calif.) can be used to evaluate the encapsulation of RNA by the transfer vehicle composition. The samples are diluted to a concentration of approximately 5 μg/mL or 1 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2-4% Triton X-100 solution is added to the wells. The plate is incubated at a temperature of 37° C. for 15 minutes. The RIBOGREEN® reagent is diluted 1:100 or 1:200 in TE buffer, and 100 μL of this solution is added to each well. The fluorescence intensity can be measured using a fluorescence plate reader (Wallac Victor 1420 Multilabel Counter; Perkin Elmer, Waltham, Mass.) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

### Example 70B: RNA Encapsulation, Total Flux, and Percent Expression In Vitro for Ionizable Hpid:DOPE:Cholesterol:DSPE-PEG(2000) Formulation Ratio of 62:4:33:1

Lipid nanoparticles were formulated using Lipid 27, 26, 46, or 45 from Table 10a in a ionizable lipid:DOPE:cholesterol:DSPE-PEG(2000) formulation ratio of 62:4:33:1 mol %, and encapsulate the RNA molecule at a lipid-nitrogen-to-phosphate ratio (N:P) of 4.5. Expression of the RNA was present in all formulations. There was a greater total flux and percent expression within the spleen as shown in FIGS. 72A and 72B respectively.

| | | | | Ionizable lipid: | | | |
|---|---|---|---|---|---|---|---|
| | | | | Helper lipid: | | | |
| | | | | Cholesterol: | Z- | | RNA |
| | Ionizable | Helper | | PEG-lipid | Average | | Encapsulation |
| Formulation | lipid | lipid | PEG-lipid | (mol %) | (nm) | PDI | Efficiency (%) |
| 10a-27 (4.5D) | Table 10a, | DOPE | DSPE- | 62:4:33:1 | 71 | 0.02 | 94 |
| | Lipid 27 | | PEG(2000) | | | | |
| 10a-26 (4.5D) | Table 10a, | DOPE | DSPE- | 62:4:33:1 | 71 | 0.04 | 92 |
| | Lipid 26 | | PEG(2000) | | | | |
| 10a-46 (4.5D) | Table 10a, | DOPE | DSPE- | 62:4:33:1 | 110 | 0.1 | 93 |
| | Lipid 26 | | PEG(2000) | | | | |
| 10a-45 (4.5D) | Table 10a, | DOPE | DSPE- | 62:4:33:1 | 157 | 0.13 | 83 |
| | Lipid 25 | | PEG(2000) | | | | |

### Example 70C: RNA Encapsulation, Total Flux, and Percent Expression In Vitro for Ionizable Hpid:DOPE:Cholesterol:DMG-PEG(2000) Formulation Ratio of 50:10:38.5:1.5

Lipid nanoparticles were formulated using Lipid 46 or 45 from Table 10a in a ionizable lipid:DOPE:cholesterol:DMG-PEG(2000) formulation ratio of 50:10:38.5:1.5 mol %, and encapsulate the RNA molecule at a lipid-nitrogen-to-phosphate ratio (N:P) of 5.7. Expression of the RNA was present in all formulations. There was a greater total flux and percent expression within the spleen as shown in FIGS. 72C and 72D respectively.

| | | | | Ionizable lipid: | | | |
|---|---|---|---|---|---|---|---|
| | | | | DOPE: | | | |
| | | | | Cholesterol: | Z- | | RNA |
| | Ionizable | Helper | | PEG-lipid | Average | | Encapsulation |
| Formulation | lipid | lipid | PEG-lipid | (mol %) | (nm) | PDI | Efficiency (%) |
| 10a-45 (5.7A) | Table 10a, | DOPE | DMG- | 50:10:38.5:1.5 | 74 | 0.04 | 95 |
| | Lipid 45 | | PEG(2000) | | | | |
| 10a-46 (5.7A) | Table 10a, | DOPE | DMG- | 50:10:38.5:1.5 | 84 | 0.04 | 96 |
| | Lipid 46 | | PEG(2000) | | | | |

### Example 70D: RNA Encapsulation, Total Flux, and Percent Expression In Vitro for Ionizable Lipid:DOPE:Cholesterol:DMG-PEG(2000) Formulation Ratio of 50:10:38.5:1.5 or for Ionizable Lipid:DSPC:Cholesterol:C₁₄-PEG(2000) Formulation Ratio 35:16:46.2.5

Lipid nanoparticles were formulated using Lipid 45 or 46 from Table 10a in an ionizable lipid:DOPE:cholesterol:DMG-PEG(2000) formulation ratio of 50:10:38.5:1.5 mol % or in an ionizable lipid:DSPC:cholesterol:C₁₄-PEG(2000) formulation ratio of 35:16:46.2.5 mol %, and encapsulate the RNA molecule at a lipid-nitrogen-to-phosphate ratio (N:P) of 5.7 or 4.5. Expression of the RNA was present in all formulations. There was a greater total flux and percent expression within the spleen as shown in FIGS. 72E and 72F respectively.

| | | | | Ionizable lipid: | | | |
|---|---|---|---|---|---|---|---|
| | | | | Helper lipid: | | | |
| | | | | Cholesterol: | Z- | | RNA |
| | Ionizable | Helper | | PEG-lipid | Average | | Encapsulation |
| Formulation | lipid | lipid | PEG-lipid | (mol %) | (nm) | PDI | Efficiency (%) |
| 10a-45 DSPC | Table 10a, | DSPC | C₁₄- | 35:16:46.2.5 | 56 | 0.22 | 94 |
| (5.7E) | Lipid 45 | | PEG(2000) | | | | |
| 10a-46 DSPC | Table 10a, | DSPC | C₁₄- | 35:16:46.2.5 | 68 | 0.02 | 95 |
| (5.7E) | Lipid 46 | | PEG(2000) | | | | |
| 10a-46 | Table 10a, | DOPE | DMG- | 50:10:38.5:1.5 | 91 | 0.13 | 93 |
| (4.5A) | Lipid 46 | | PEG(2000) | | | | |
| 10a-46 | Table 10a, | DOPE | DMG- | 50:10:38.5:1.5 | 76 | 0.06 | 93 |
| (5.7A) | Lipid 46 | | PEG(2000) | | | | |

### Example 70E: RNA Encapsulation, Total Flux, and Percent Expression In Vitro for Ionizable Lipid:DOPE:Cholesterol:DSPE-PEG(2000) Formulation Ratio of 62:4:33:1 or for Ionizable Lipid:DSPC:Cholesterol:DMG-PEG(2000) Formulation Ratio of 50:10:38.5:1.5

Lipid nanoparticles were formulated using Lipid 26 or 27 from Table 10a in a ionizable lipid:DOPE:cholesterol:DSPE-PEG(2000) formulation ratio of 62:4:33:1 mol % (encapsulating the RNA molecule at a N:P ratio of 4.5) or ionizable lipid:DSPC:cholesterol:DMG-PEG(2000) formulation ratio of 50:10:38.5:1.5 mol % (encapsulating the RNA molecule at a N:P ratio of 5.7). Expression of the RNA was present in all formulations. There was a greater total flux and percent expression within the spleen as shown in FIGS. 72G and 72H respectively.

| | | | | Ionizable lipid: | | | |
|---|---|---|---|---|---|---|---|
| | | | | Helper lipid: | | | |
| | | | | Cholesterol: | Z- | | Encapsulation |
| | Ionizable | Helper | | PEG-lipid | Average | | Efficiency (EE) |
| Formulation | lipid | lipid | PEG-lipid | (mol %) | (nm) | PDI | (%) |
| 10a-27 | Table 10a, | DOPE | DSPE- | 62:4:33:1 | 82 | 0.06 | 94 |
| (4.5D) | Lipid 27 | | PEG(2000) | | | | |
| 10a-26 | Table 10a, | DOPE | DSPE- | 62:4:33:1 | 68 | 0.08 | 91 |
| (4.5D) | Lipid 26 | | PEG(2000) | | | | |
| 10a-27 DSPC | Table 10a, | DSPC | DMG- | 50:10:38.5:1.5 | 79 | 0.06 | 96 |
| (5.7A) | Lipid 27 | | PEG(2000) | | | | |
| 10a-26 DSPC | Table 10a, | DSPC | DMG- | 50:10:38.5:1.5 | 79 | 0.05 | 93 |
| (5.7A) | Lipid 26 | | PEG(2000) | | | | |

### Example 70F: RNA Encapsulation, Total Flux, and Percent Expression In Vitro for Ionizable Lipid:DOPE:Cholesterol:DMG-PEG(2000) Formulation Ratio of 50:10:38.5:1.5

Lipid nanoparticles were formulated using Lipid 26, 27, 130 from Table 10a and/or Lipid III-1 from Table 3 in a ionizable lipid:DSPC:cholesterol:DMG-PEG(2000) formulation ratio of 50:10:38.5:1.5 mol %, and encapsulate the RNA molecule at a N:P ratio of 5.7. Expression of the RNA was present in all formulations. There was a greater total flux and percent expression within the liver as shown in FIGS. 72I and 72J respectively.

TNS and the particle's pKₐ was also calculated. 5 μL of 60 μg/mL 2-(p-toluidino) naphthalene-6-sulfonic acid (TNS) and 5 μL of 30 μg of RNA/mL lipid nanoparticles were added in to wells with HEPES buffer ranging from pH 2-12. The mixture was then shaken at room temperature for 5 minutes, and read for fluorescence (excitation 322 nm, emission 431 nm) using a plate reader. The inflection point of the fluorescence signal was calculated to determine the particle's pKₐ.

| | | | | Ionizable lipid: | | | | | |
|---|---|---|---|---|---|---|---|---|---|
| | | | | Helper lipid: | | | | | |
| | | | | Cholesterol: | | | | | |
| | Ionizable | Helper | | PEG-lipid | Z- | | EE | Conc. | |
| Formulation | lipid | lipid | PEG-lipid | (mol %) | Average | PDI | (%) | (ug/mL) | pKa |
| 10a-27/III-1 | Table 10a, | DOPE | DMG- | 50:10:38.5:1.5 | 74 | 0 | 96 | 55.8 | 7.4 |
| (5.7A) | Lipid 27/ | | PEG(2000) | | | | | | |
| | Table 3, | | | | | | | | |
| | Lipid III-1 | | | | | | | | |
| | 3:1 ratio | | | | | | | | |
| 10a-27III-1 | Table 10a, | DOPE | DMG- | 50:10:38.5:1.5 | 85 | 0.1 | 93 | 51.9 | 6.3 |
| (5.7A) | Lipid 27/ | | PEG(2000) | | | | | | |
| | Table 3, | | | | | | | | |
| | Lipid III-1 | | | | | | | | |
| | 1:3 ratio | | | | | | | | |
| 10a-26/III-1 | Table 10a, | DOPE | DMG- | 50:10:38.5:1.5 | 87 | 0.1 | 94 | 51.7 | 6.8 |
| (5.7A) | Lipid 26/ | | PEG(2000) | | | | | | |
| | Table 3, | | | | | | | | |
| | Lipid III-1 | | | | | | | | |
| | 3:1 ratio | | | | | | | | |
| 10a-26/III-1 | Table 10a, | DOPE | DMG- | 50:10:38.5:1.5 | 97 | 0.1 | 90 | 53.1 | 6.2 |
| (5.7A) | Lipid 26/ | | PEG(2000) | | | | | | |
| | Table 3, | | | | | | | | |
| | Lipid III-1 | | | | | | | | |
| | 1:3 ratio | | | | | | | | |
| 10a-130 | Table 10a, | DOPE | DMG- | 50:10:38.5:1.5 | 60 | 0.1 | 89 | 53.8 | 6.7 |
| (5.7A) | Lipid 130 | | PEG(2000) | | | | | | |

### Example 70G: RNA Encapsulation, Total Flux, and Percent Expression In Vitro for Ionizable Lipid:DOPE:Cholesterol:DSPE-PEG(2000) Formulation Ratio of 62:4:33:1 or for Ionizable Lipid:DSPC:Cholesterol:DMG-PEG(2000) Formulation Ratio of 50:10:38.5:1.5

Lipids nanoparticles were formulated using Lipid 139 from Table 10a in a ionizable lipid:DOPE:cholesterol:DSPE-PEG(2000) formulation ratio of 62:4:33:1 mol % (encapsulating the RNA molecule at a N:P ratio of 4.5) or ionizable lipid:DOPE:cholesterol:DMG-PEG(2000) formulation ratio of 50:10:38.5:1.5 mol % (encapsulating the RNA molecule at a N:P ratio of 5.7). Expression of the RNA was present in all formulations. There was a greater total flux and percent expression within the liver as shown in FIGS. 72K and 72L respectively.

| | | | | Ionizable lipid: | | | |
|---|---|---|---|---|---|---|---|
| | | | | Helper lipid: | | | |
| | | | | Cholesterol: | Z- | | |
| | Ionizable | Helper | | PEG-lipid | Average | | EE |
| Formulation | lipid | lipid | PEG-lipid | (mol %) | (nm) | PDI | (%) |
| 10a-139 | Table 10a, | DOPE | DSPE- | 62:4:33:1 | 93 | 0.01 | 79 |
| (4.5D) | Lipid 139 | | PEG(2000) | | | | |
| 10a-139 | Table 10a, | DSPC | DMG- | 50:10:38.5:1.5 | 122 | 0.02 | 95 |
| (5.7A) | Lipid 139 | | PEG(2000) | | | | |

### INCORPORATION BY REFERENCE

All publications, patents, and patent applications mentioned in this specification are herein incorporated by reference to the same extent as if each individual publication, patent, or patent application was specifically and individually indicated as being incorporated by reference herein.

What is claimed is:

1. A pharmaceutical composition comprising:
a. a circular RNA polynucleotide, and
b. a transfer vehicle comprising an ionizable lipid represented by Formula (1): 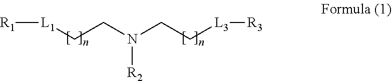
wherein:
each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkyl sulfoxidealkyl, alkyl sulfonyl, and alkylsulfonealkyl; and
R₂ is selected from a group consisting of:

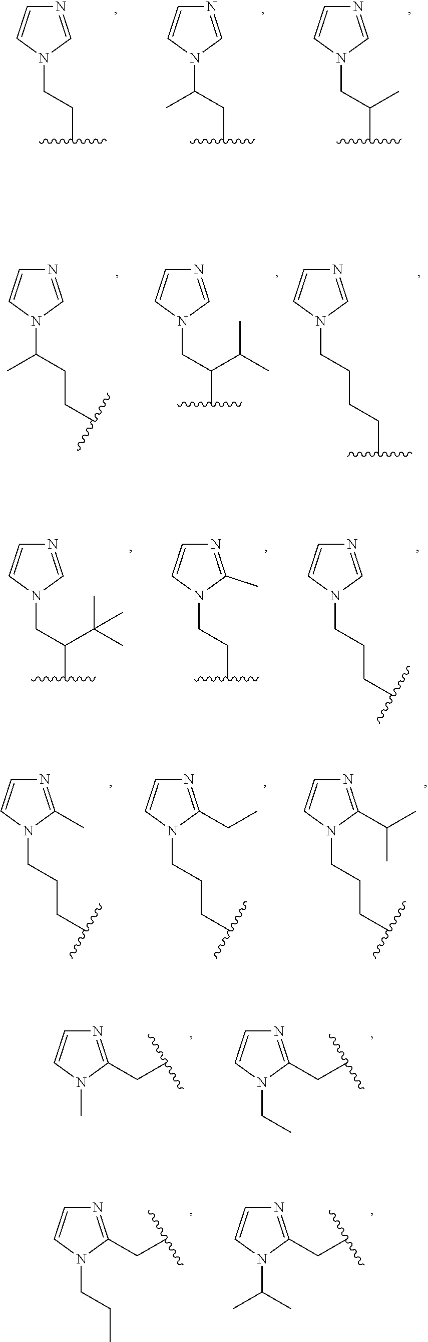

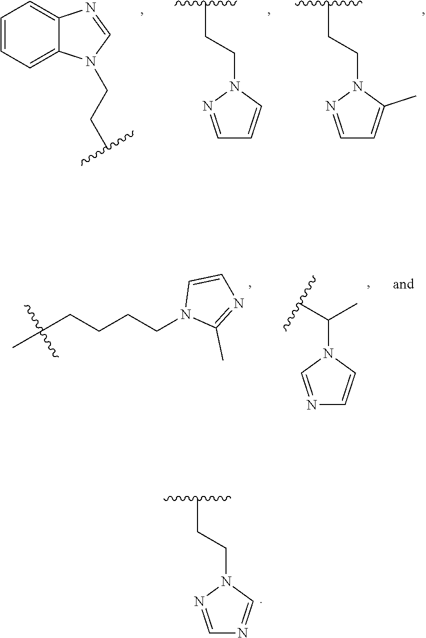

2. The pharmaceutical composition of claim 1, wherein the circular RNA polynucleotide is encapsulated in the transfer vehicle.

3. The pharmaceutical composition of claim 2, wherein the circular RNA polynucleotide is encapsulated in the transfer vehicle with an encapsulation efficiency of at least 80%.

4. The pharmaceutical composition of any one of claims 1-3, wherein R₁ and R₃ are each independently selected from a group consisting of:

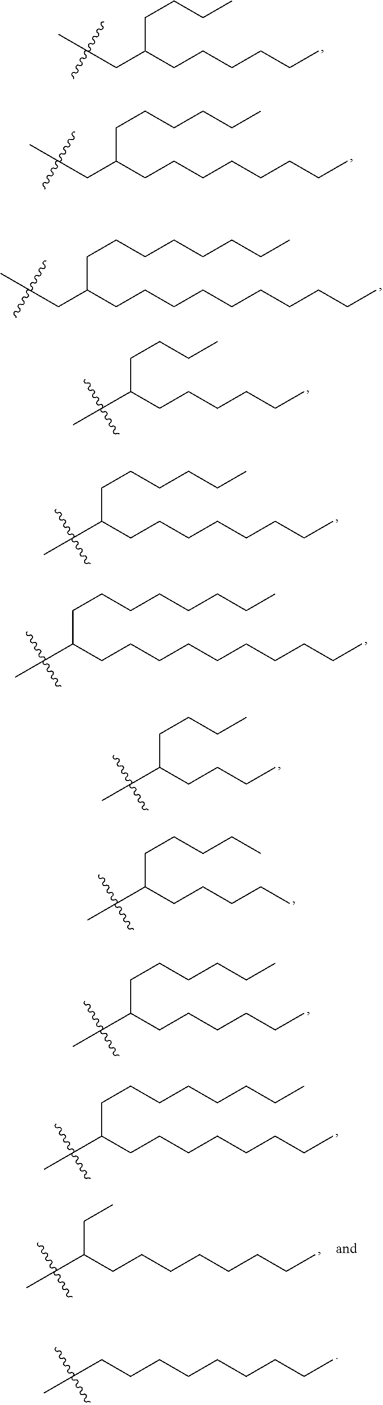

5. The pharmaceutical composition of any one of claims 1-4, wherein R₁ and R₃ are the same.

6. The pharmaceutical composition of any one of claims 1-4, wherein R₁ and R₃ are different.

7. The pharmaceutical composition of any one of claims 1-6, wherein the transfer vehicle has a diameter of about 56 nm or larger.

8. The pharmaceutical composition of claim 7, wherein the transfer vehicle has a diameter of about 56 nm to about 157 nm.

9. The pharmaceutical composition of any one of claims 1-8, wherein the ionizable lipid is represented by Formula (1-1) or Formula (1-2):

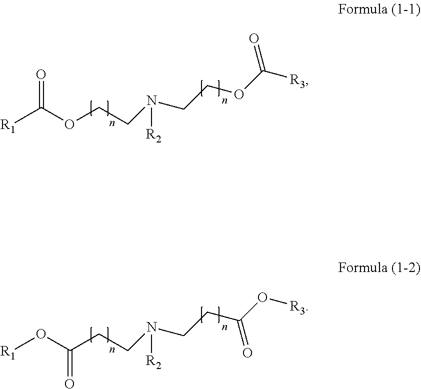

10. The pharmaceutical composition of any one of claims 1-9, wherein the ionizable lipid is selected from the group consisting of:

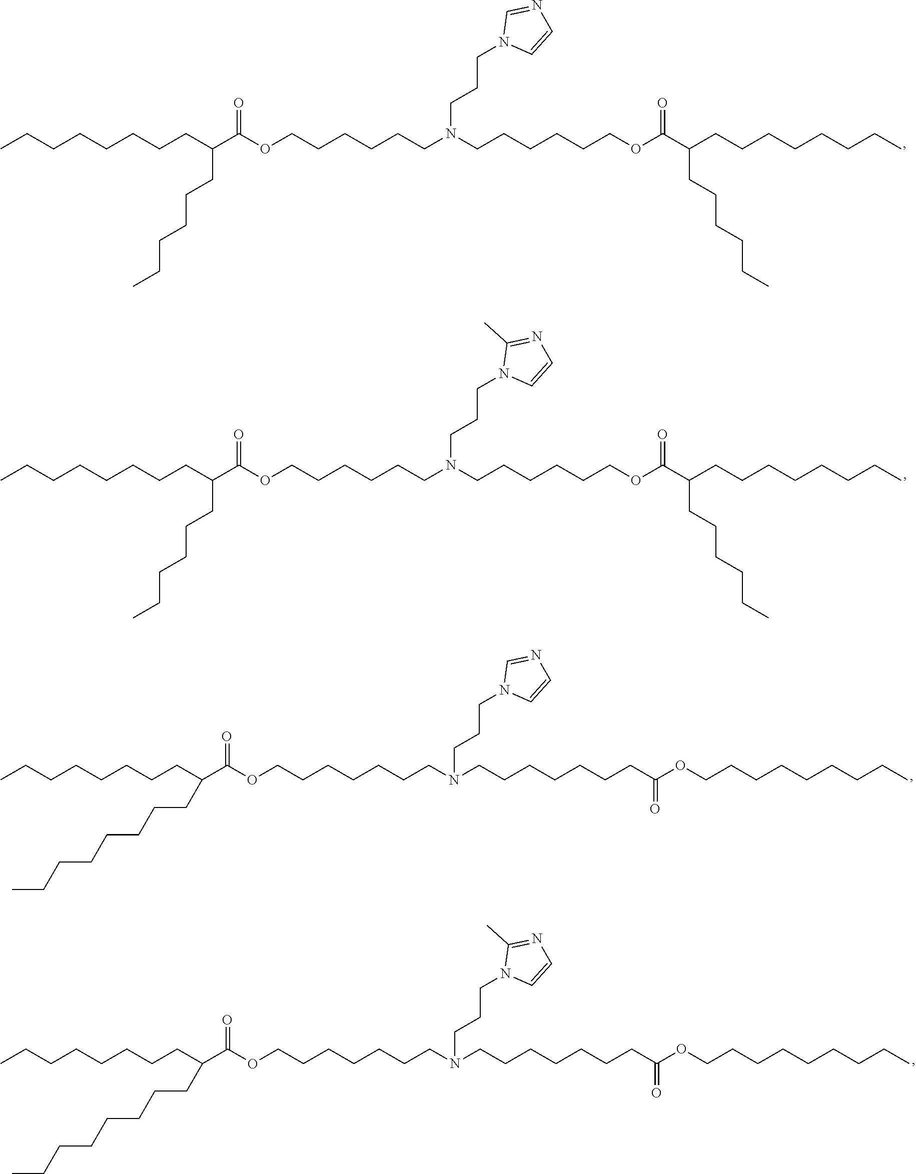

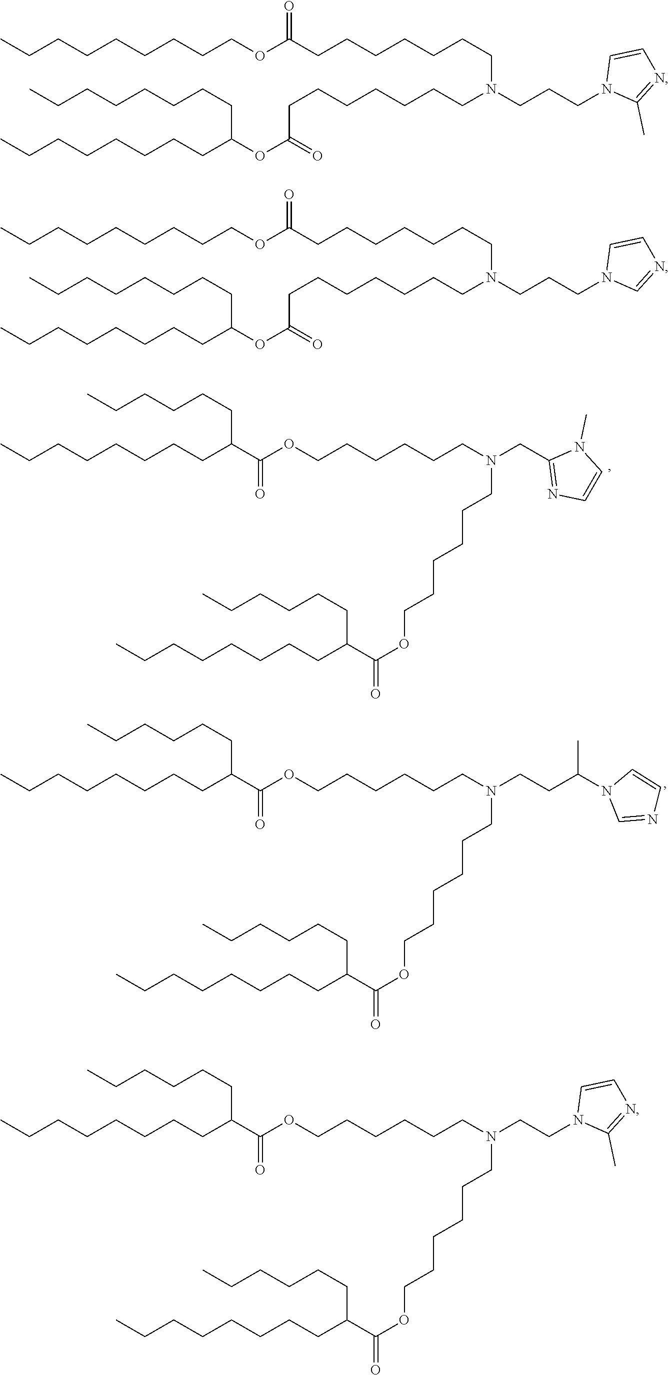 [SMILES: CCCCCCCCCOC(=O)CCCCCCCN(CCCCCCCC(=O)OC(CCCCCCCC)CCCCCCCC)CCCn1ccnc1C ; CCCCCCCCCOC(=O)CCCCCCCN(CCCCCCCC(=O)OC(CCCCCCCC)CCCCCCCC)CCCn1ccnc1 ; CCCCCCCCC(CCCCCC)C(=O)OCCCCCCN(CCCCCCOC(=O)C(CCCCCC)CCCCCCCC)Cc1nccn1C ; CCCCCCCCC(CCCCCC)C(=O)OCCCCCCN(CCCCCCOC(=O)C(CCCCCC)CCCCCCCC)CCC(C)n1ccnc1 ; CCCCCCCCC(CCCCCC)C(=O)OCCCCCCN(CCCCCCOC(=O)C(CCCCCC)CCCCCCCC)CCn1ccnc1C]

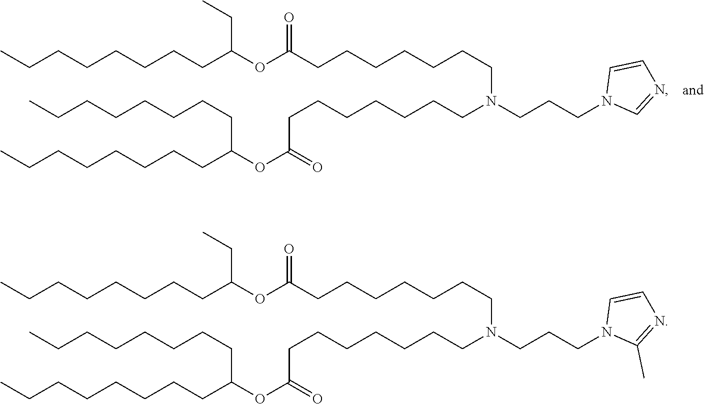 [SMILES: CCCCCCCCC(CC)OC(=O)CCCCCCCN(CCCCCCCC(=O)OC(CCCCCCCC)CCCCCCCC)CCCn1ccnc1 ; "and" ; CCCCCCCCC(CC)OC(=O)CCCCCCCN(CCCCCCCC(=O)OC(CCCCCCCC)CCCCCCCC)CCCn1ccnc1C]

11. A pharmaceutical composition comprising:
a. a circular RNA polynucleotide, and
b. a transfer vehicle comprising an ionizable lipid represented by Formula (2): 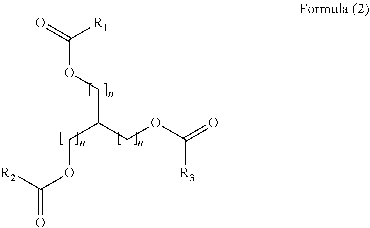
wherein:
each n is independently an integer from 1-15;
R₁ and R₂ are each independently selected from a group consisting of: 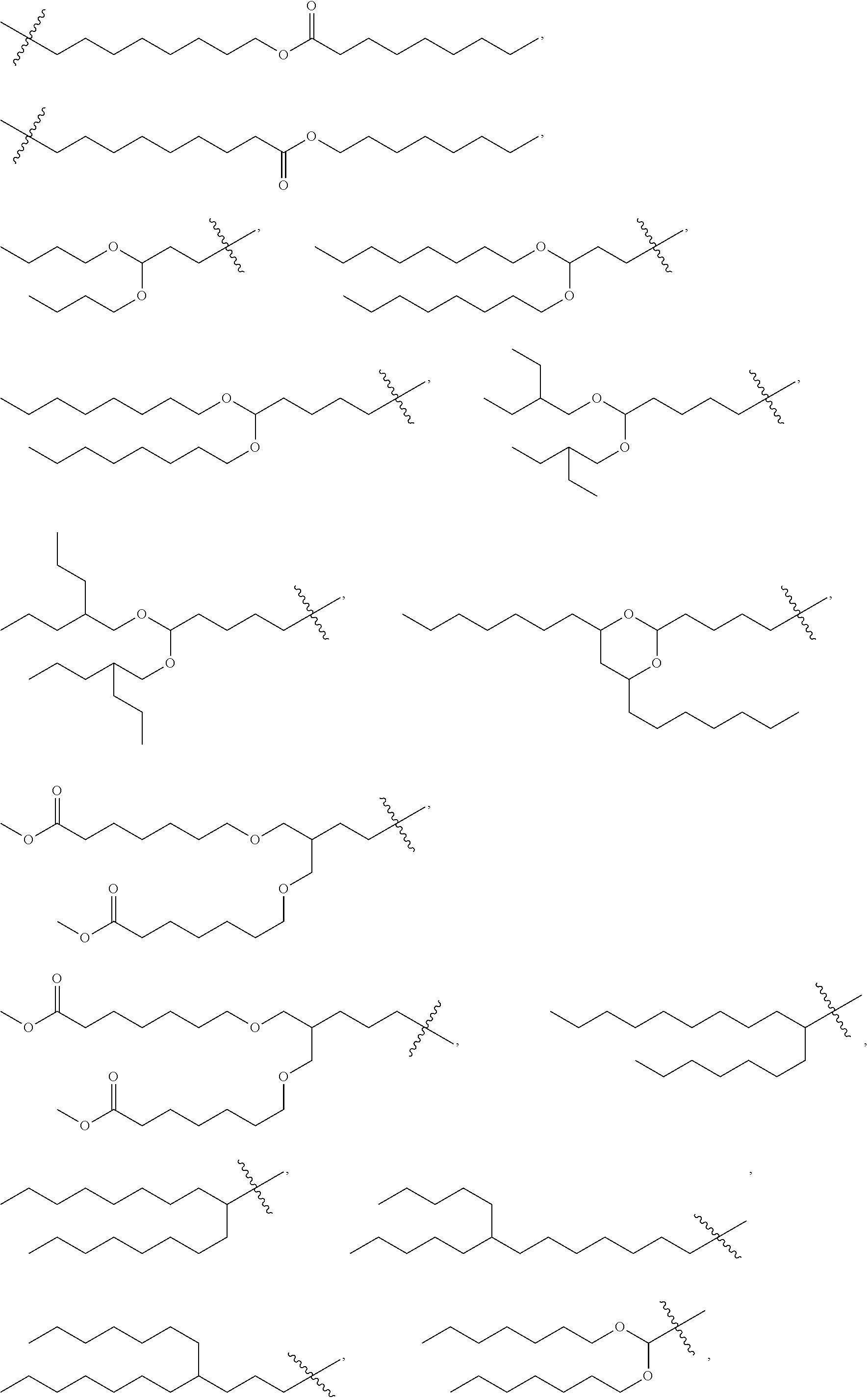 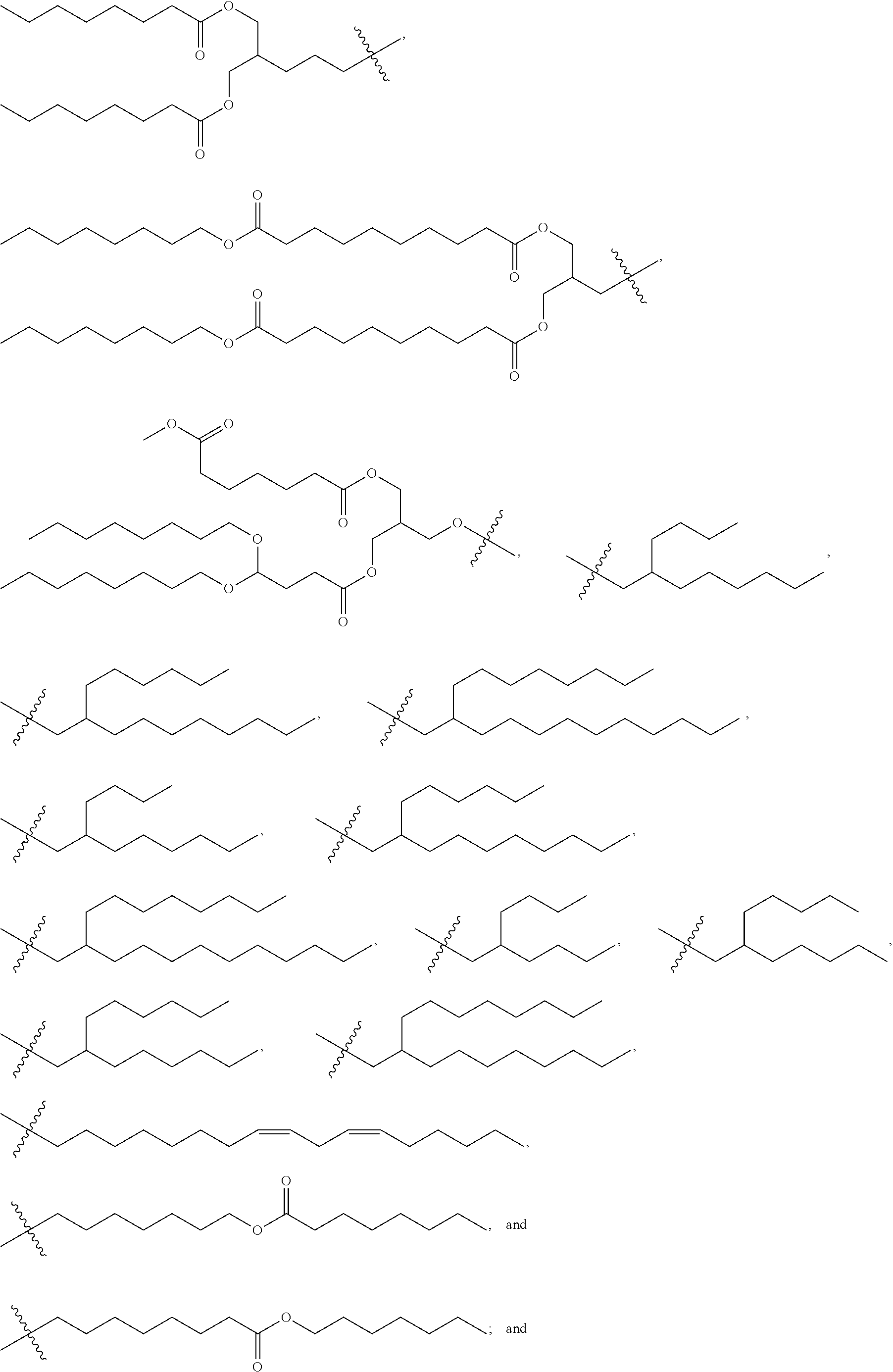
R₃ is selected from a group consisting of:

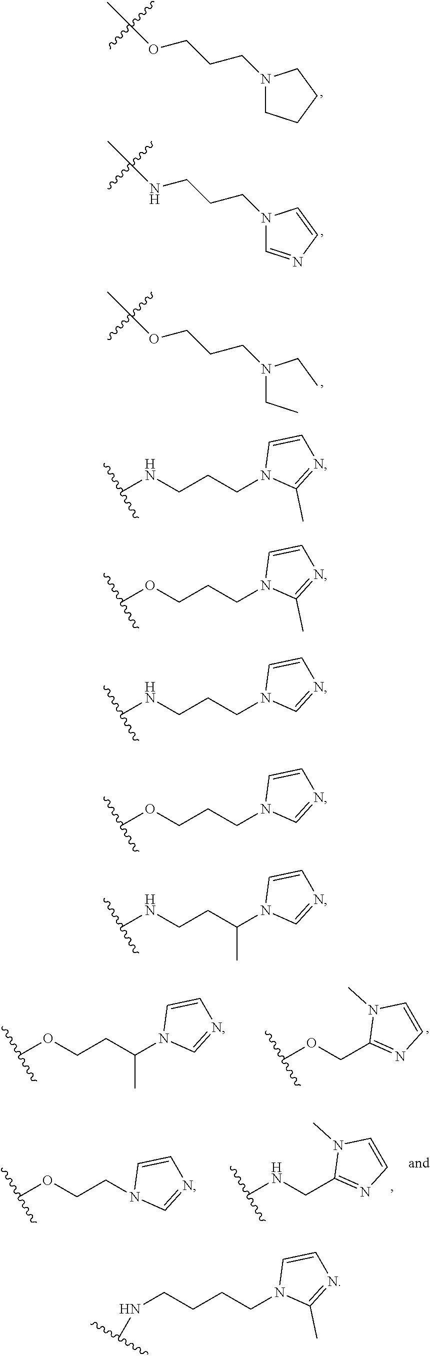

12. The pharmaceutical composition of claim 11, wherein the circular RNA polynucleotide is encapsulated in the transfer vehicle.

13. The pharmaceutical composition of claim 12, wherein the circular RNA polynucleotide is encapsulated in the transfer vehicle with an encapsulation efficiency of at least 80%.

14. The pharmaceutical composition of any one of claims 11-13, wherein the ionizable lipid is selected from the group consisting of:

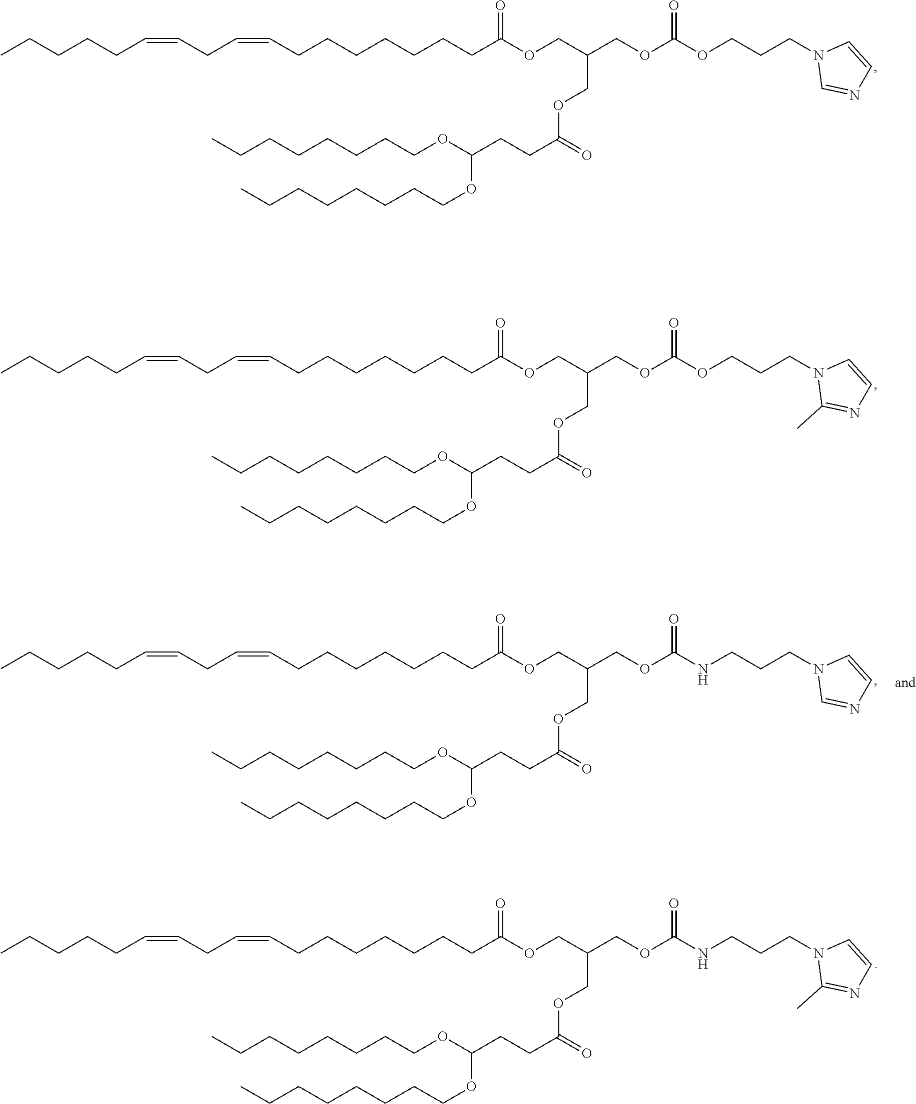 [SMILES: CCCCC/C=C\C/C=C\CCCCCCCC(=O)OCC(COC(=O)CCC(OCCCCCCCC)OCCCCCCCC)COC(=O)OCCCn1ccnc1 ; CCCCC/C=C\C/C=C\CCCCCCCC(=O)OCC(COC(=O)CCC(OCCCCCCCC)OCCCCCCCC)COC(=O)OCCCn1ccnc1C ; CCCCC/C=C\C/C=C\CCCCCCCC(=O)OCC(COC(=O)CCC(OCCCCCCCC)OCCCCCCCC)COC(=O)NCCCn1ccnc1 ; CCCCC/C=C\C/C=C\CCCCCCCC(=O)OCC(COC(=O)CCC(OCCCCCCCC)OCCCCCCCC)COC(=O)NCCCn1ccnc1C]

15. A pharmaceutical composition comprising:
a. a circular RNA polynucleotide, and
b. a transfer vehicle comprising an ionizable lipid represented by Formula (3): 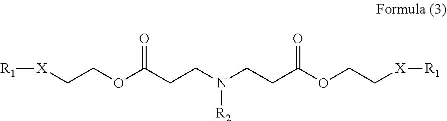
wherein:
X is selected from —O—, —S—, or —OC(O)—*, wherein * indicates the attachment point to R₁;
R₁ is selected from a group consisting of: 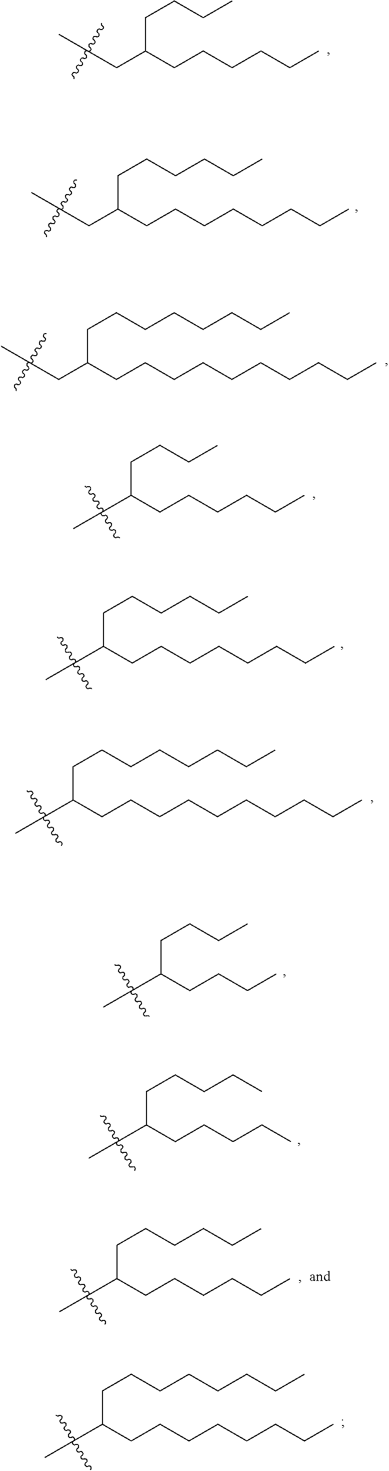
and
R₂ is selected from a group consisting of:

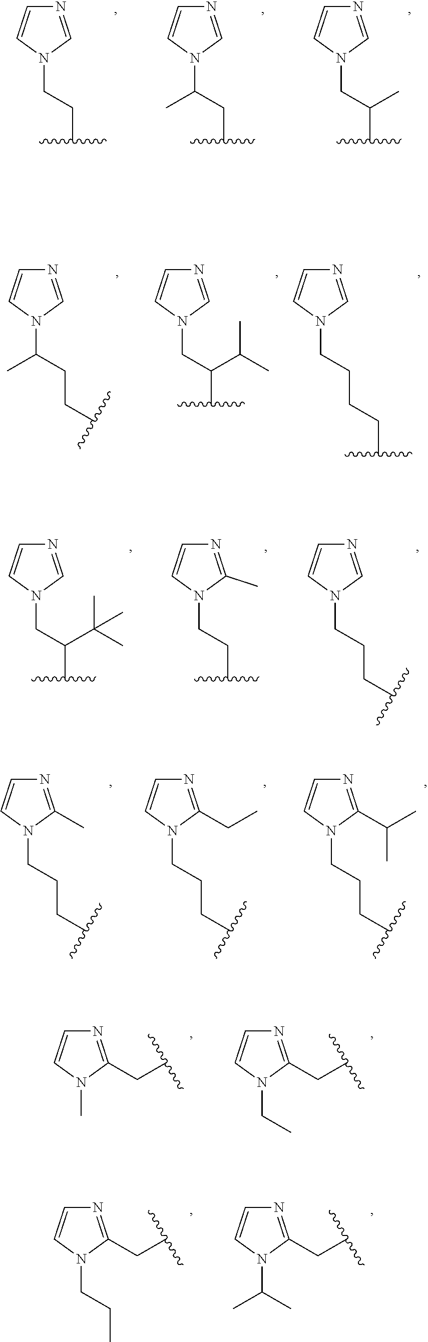

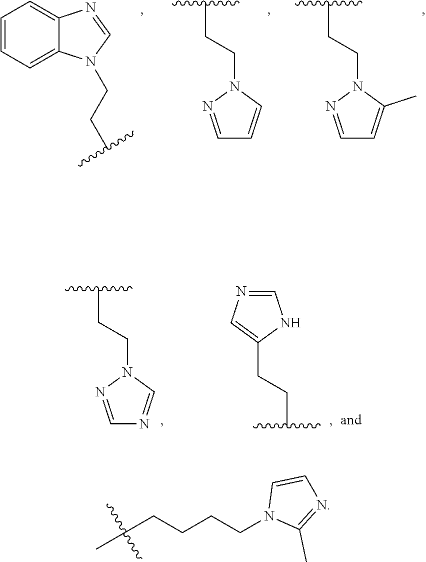

16. The pharmaceutical composition of claim 15, wherein the circular RNA polynucleotide is encapsulated in the transfer vehicle.

17. The pharmaceutical composition of claim 16, wherein the circular RNA polynucleotide is encapsulated in the transfer vehicle with an encapsulation efficiency of at least 80%.

18. The pharmaceutical composition of any one of claims 15-17, wherein the ionizable lipid is represented by Formula (3-1), Formula (3-2), or Formula (3-3):

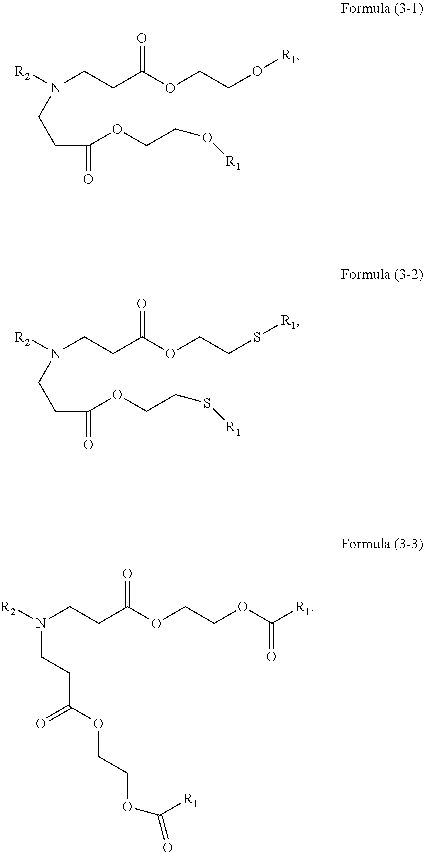

19. The pharmaceutical composition of any one of claims 15-18, wherein the ionizable lipid is selected from the group consisting of:

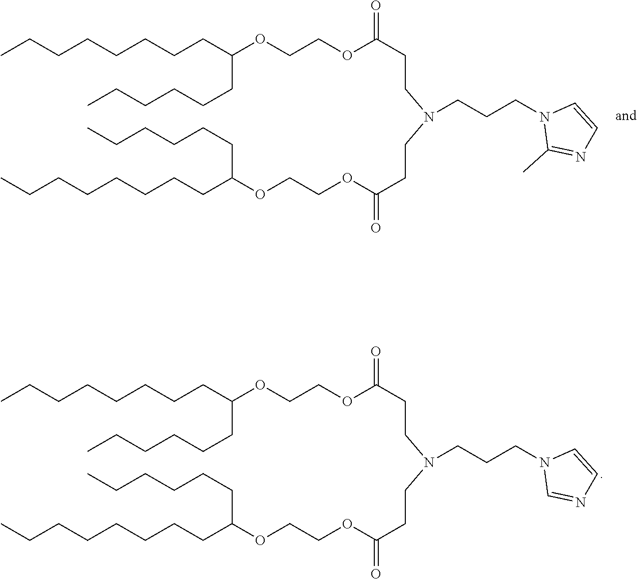 [SMILES: CCCCCCCCC(CCCCCC)OCCOC(=O)CCN(CCCn1ccnc1C)CCC(=O)OCCOC(CCCCCC)CCCCCCCC ; CCCCCCCCC(CCCCCC)OCCOC(=O)CCN(CCCn1ccnc1)CCC(=O)OCCOC(CCCCCC)CCCCCCCC]

20. A pharmaceutical composition comprising:
a. a circular RNA polynucleotide, and
b. a transfer vehicle comprising an ionizable lipid represented by Formula (4): 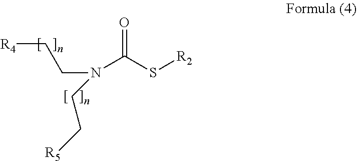
wherein:
each n is independently an integer from 2-15; and
R₂ is as defined in claim 2.

21. The pharmaceutical composition of claim 20, wherein the circular RNA polynucleotide is encapsulated in the transfer vehicle.

22. The pharmaceutical composition of claim 21, wherein the circular RNA polynucleotide is encapsulated in the transfer vehicle with an encapsulation efficiency of at least 80%.

23. A pharmaceutical composition comprising:
a. a circular RNA polynucleotide, and
b. a transfer vehicle comprising an ionizable lipid represented by Formula (6): 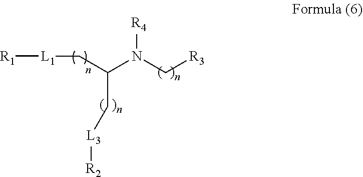
wherein:
each n is independently an integer from 0-15;
L₁ and L₃ are each independently —OC(O)—* or —C(O)O—*, wherein “*” indicates the attachment point to R₁ or R₃;
R₁ and R₂ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkyl sulfoxidealkyl, alkyl sulfonyl, and alkylsulfonealkyl;
R₃ is selected from a group consisting of: 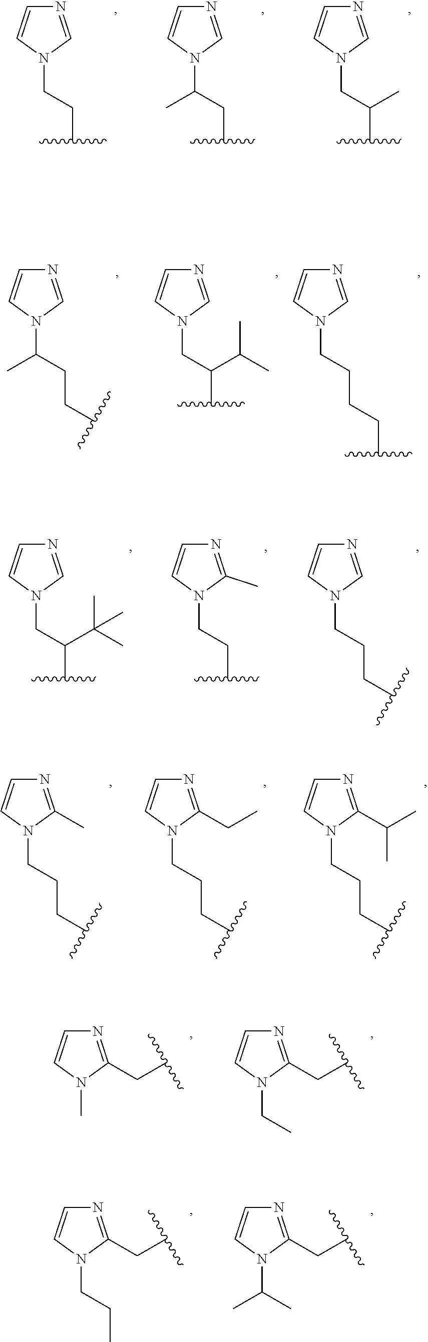 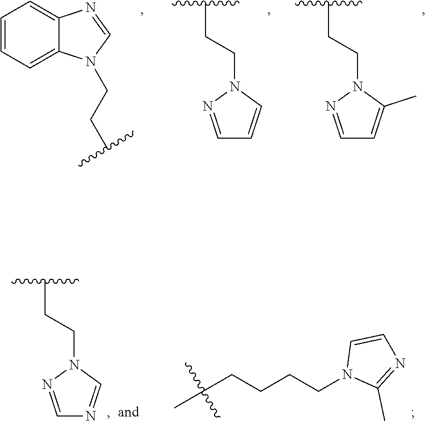
and
R₄ is a linear or branched C₁-C₁₅ alkyl or C₁-C₁₅ alkenyl.

24. The pharmaceutical composition of claim 23, wherein the circular RNA polynucleotide is encapsulated in the transfer vehicle.

25. The pharmaceutical composition of claim 24, wherein the circular RNA polynucleotide is encapsulated in the transfer vehicle with an encapsulation efficiency of at least 80%.

26. The pharmaceutical composition of any one of claims 23-25, wherein R₁ and R₂ are each independently selected from a group consisting of:

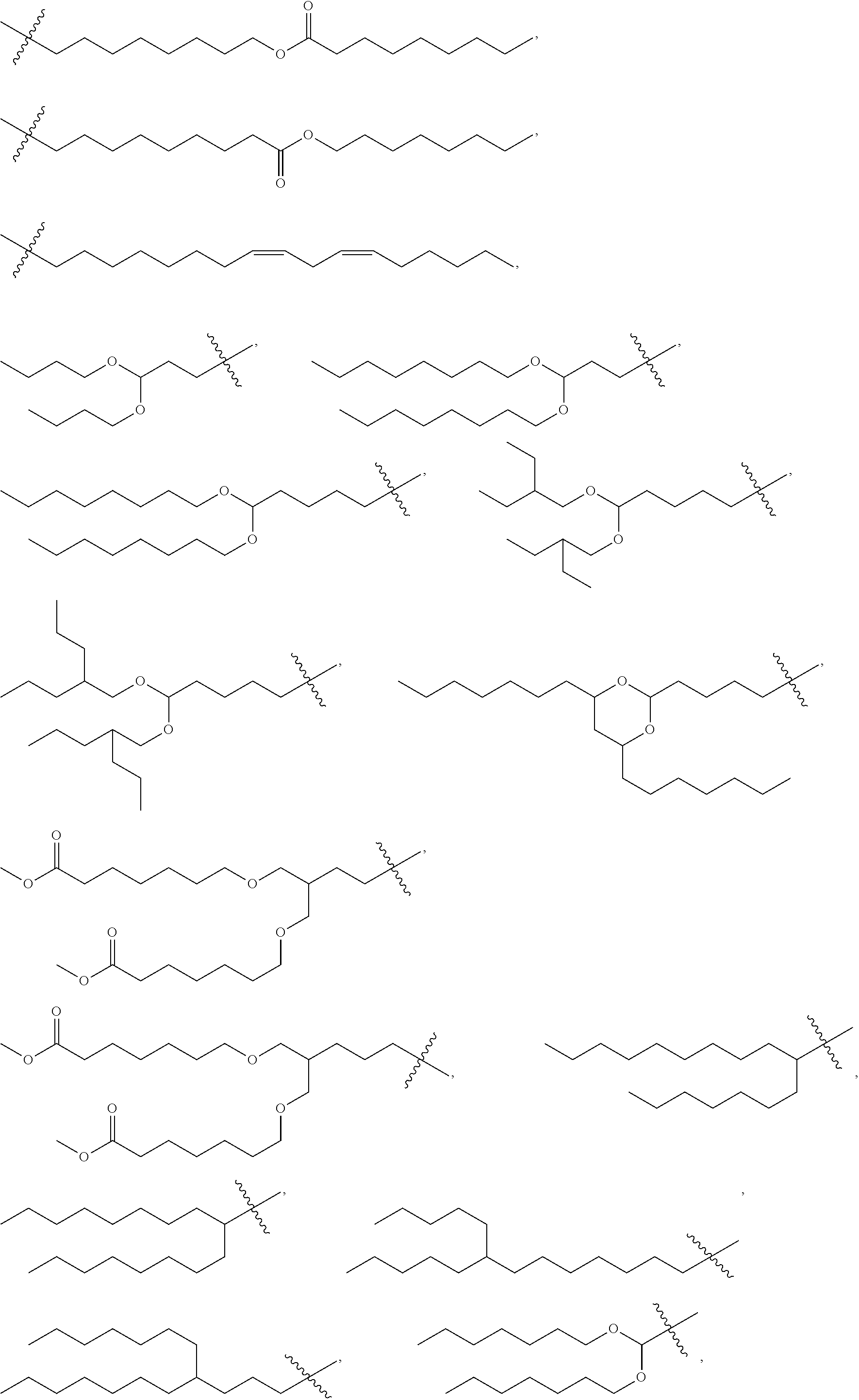

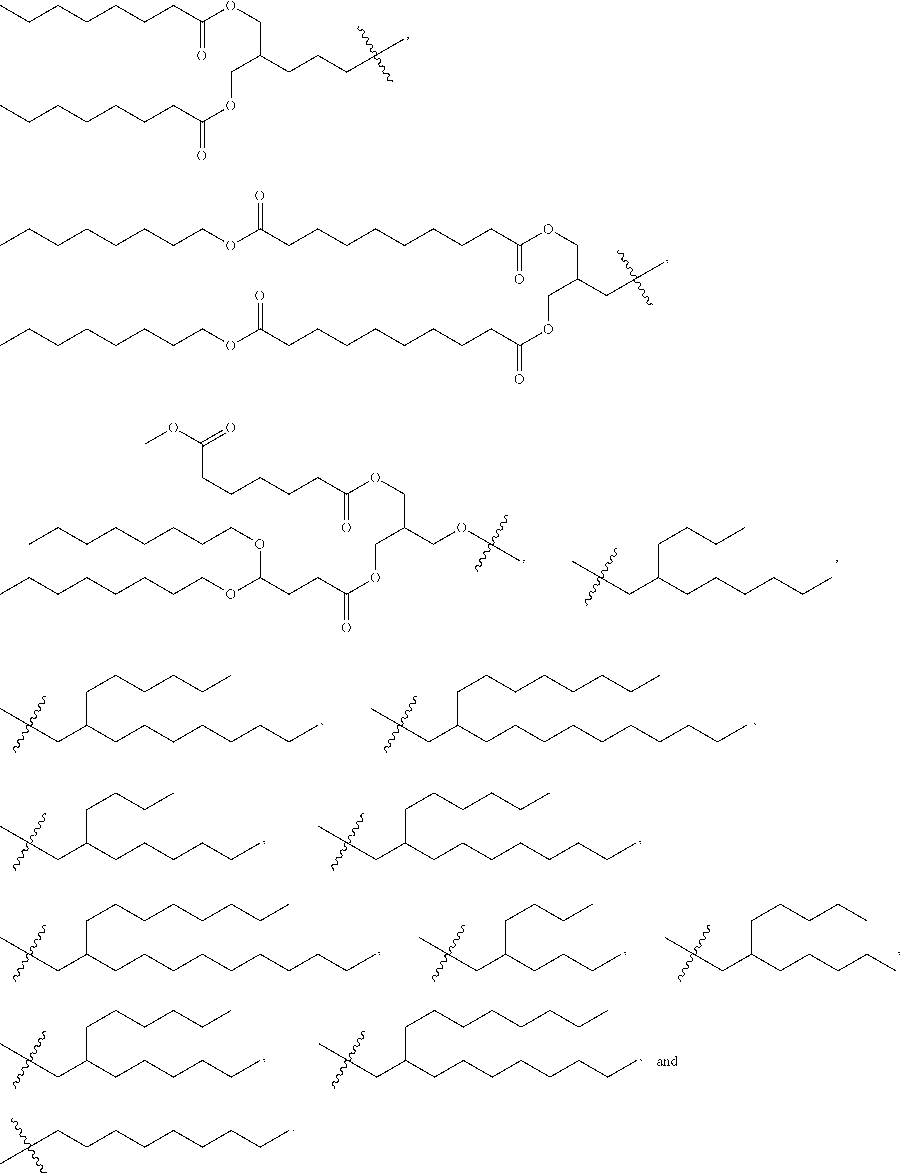

27. The pharmaceutical composition of any one of claims 23-26, wherein R₁ and R₂ are the same.

28. The pharmaceutical composition of any one of claims 23-26, wherein R₁ and R₂ are different.

29. The pharmaceutical composition of any one of claims 23-28, wherein the ionizable lipid is selected from the group consisting of:

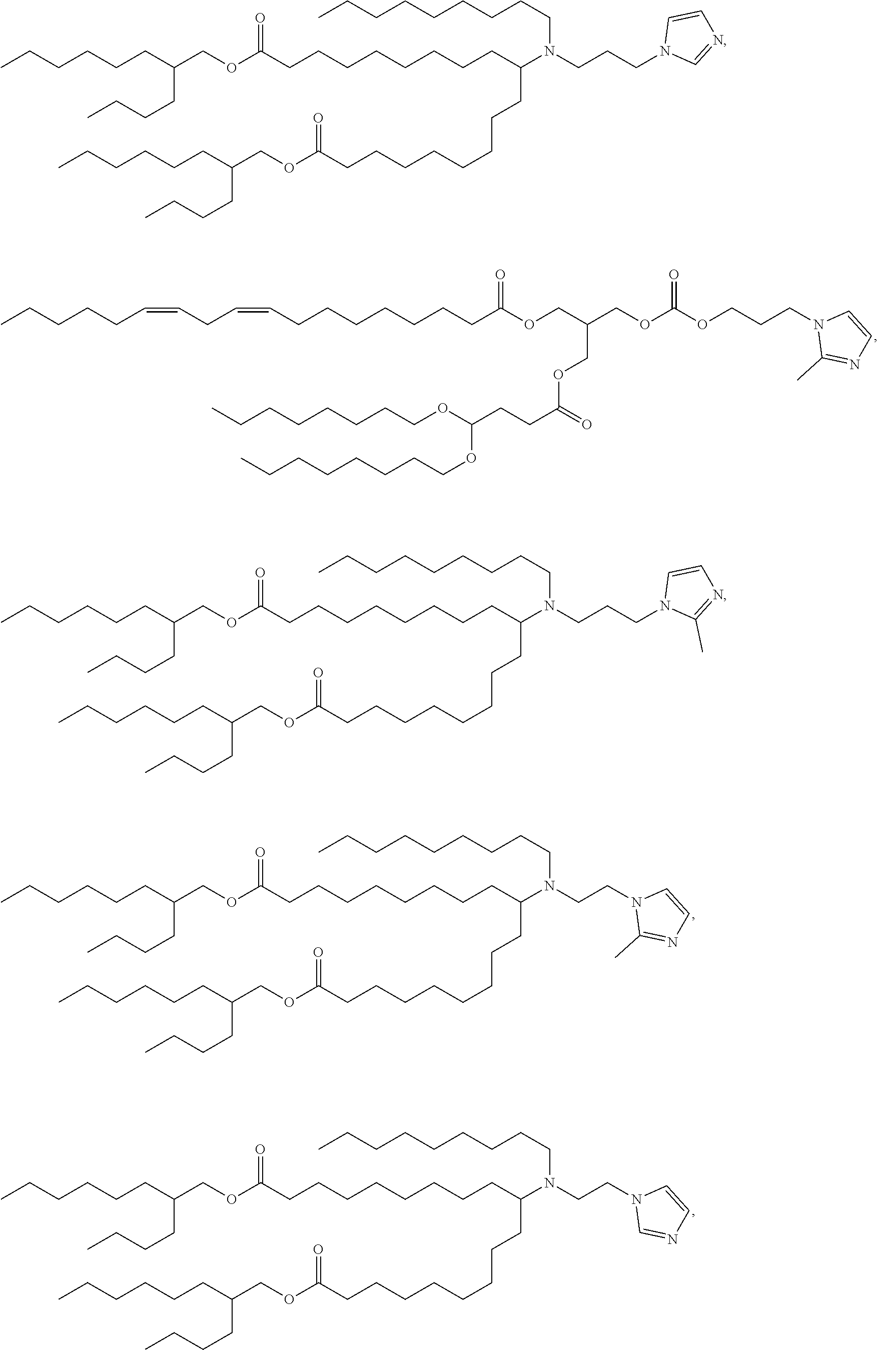 [SMILES: CCCCCCCCCN(CCCn1ccnc1)C(CCCCCCCCC(=O)OCC(CCCC)CCCCCC)CCCCCCCCC(=O)OCC(CCCC)CCCCCC ; CCCCC/C=C\C/C=C\CCCCCCCC(=O)OCC(COC(=O)CCC(OCCCCCCCC)OCCCCCCCC)COC(=O)OCCCn1ccnc1C ; CCCCCCCCCN(CCCn1ccnc1C)C(CCCCCCCCC(=O)OCC(CCCC)CCCCCC)CCCCCCCCC(=O)OCC(CCCC)CCCCCC ; CCCCCCCCCN(CCn1ccnc1C)C(CCCCCCCCC(=O)OCC(CCCC)CCCCCC)CCCCCCCCC(=O)OCC(CCCC)CCCCCC ; CCCCCCCCCN(CCn1ccnc1)C(CCCCCCCCC(=O)OCC(CCCC)CCCCCC)CCCCCCCCC(=O)OCC(CCCC)CCCCCC]

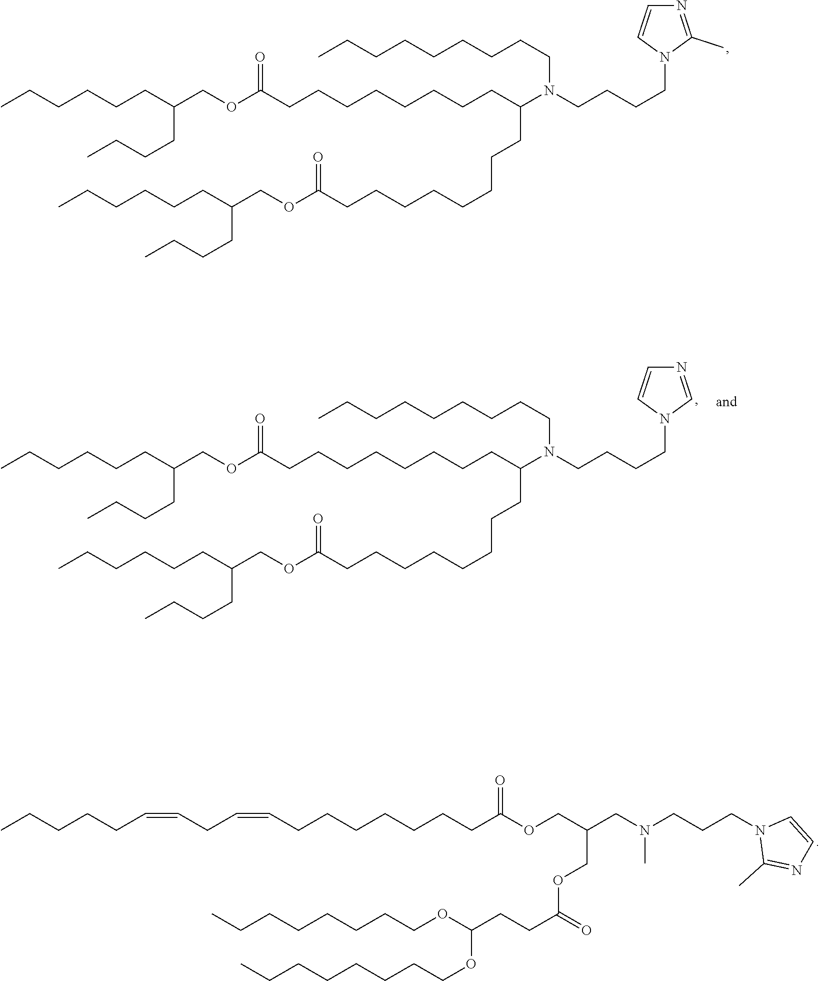

30. A pharmaceutical composition comprising:
a. a circular RNA polynucleotide, and
b. a transfer vehicle comprising an ionizable lipid selected from Table 10a.

31. The pharmaceutical composition of claim 30, wherein the circular RNA polynucleotide is encapsulated in the transfer vehicle.

32. The pharmaceutical composition of claim 31, wherein the circular RNA polynucleotide is encapsulated in the transfer vehicle with an encapsulation efficiency of at least 80%.

33. The pharmaceutical composition of any one of claims 1-32, wherein the circular RNA comprises a first expression sequence.

34. The pharmaceutical composition of claim 33, wherein the first expression sequence encodes a therapeutic protein.

35. The pharmaceutical composition of claim 33, wherein the first expression sequence encodes a cytokine or a functional fragment thereof.

36. The pharmaceutical composition of claim 33 wherein the first expression sequence encodes a transcription factor.

37. The pharmaceutical composition of claim 33, wherein the first expression sequence encodes an immune checkpoint inhibitor.

38. The pharmaceutical composition of claim 33, wherein the first expression sequence encodes a chimeric antigen receptor.

39. The pharmaceutical composition of any one of claims 1-38, wherein the circular RNA polynucleotide further comprises a second expression sequence.

40. The pharmaceutical composition of claim 39, wherein the circular RNA polynucleotide further comprises an internal ribosome entry site (IRES).

41. The pharmaceutical composition of claim 39, wherein the first and second expression sequences are separated by a ribosomal skipping element or a nucleotide sequence encoding a protease cleavage site.

42. The pharmaceutical composition of any one of claims 39-41, wherein the first expression sequence encodes a first T-cell receptor (TCR) chain and the second expression sequence encodes a second TCR chain.

43. The pharmaceutical composition of any one of claims 1-42, wherein the circular RNA polynucleotide comprises one or more microRNA binding sites.

44. The pharmaceutical composition of claim 43, wherein the microRNA binding site is recognized by a microRNA expressed in the liver.

45. The pharmaceutical composition of claim 43 or 44, wherein the microRNA binding site is recognized by miR-122.

46. The pharmaceutical composition of any one of claims 1-45, wherein the circular RNA polynucleotide comprises a first IRES associated with greater protein expression in a human immune cell than in a reference human cell.

47. The pharmaceutical composition of claim 46, wherein the human immune cell is a T cell, an NK cell, an NKT cell, a macrophage, or a neutrophil.

48. The pharmaceutical composition of claim 46 or 47, wherein the reference human cell is a hepatic cell.

49. The pharmaceutical composition of any one of claims 1-48, wherein the circular RNA polynucleotide comprises, in the following order:
a. a post-splicing intron fragment of a 3′ group I intron fragment,
b. an IRES,
c. an expression sequence, and
d. a post-splicing intron fragment of a 5′ group I intron fragment.

50. The pharmaceutical composition of claim 49, comprising a first spacer before the post-splicing intron fragment of the 3′ group I intron fragment, and a second spacer after the post-splicing intron fragment of the 5′ group I intron fragment.

51. The pharmaceutical composition of claim 50, wherein the first and second spacers each have a length of about 10 to about 60 nucleotides.

52. The pharmaceutical composition of any one of claims 1-51, wherein the circular RNA polynucleotide is made via circularization of a RNA polynucleotide comprising, in the following order:
a. a 3′ group I intron fragment,
b. an IRES,
c. an expression sequence, and
d. a 5′ group I intron fragment.

53. The pharmaceutical composition of any one of claims 1-51, wherein the circular RNA polynucleotide is made via circularization of a RNA polynucleotide comprising, in the following order:
a. a 5′ external duplex forming region,
b. a 3′ group I intron fragment,
c. a 5′ internal spacer optionally comprising a 5′ internal duplex forming region,
d. an IRES,
e. an expression sequence,
f. a 3′ internal spacer optionally comprising a 3′ internal duplex forming region,
g. a 5′ group I intron fragment, and
h. a 3′ external duplex forming region.

54. The pharmaceutical composition of any one of claims 1-51, wherein the circular RNA polynucleotide is made via circularization of a RNA polynucleotide comprising, in the following order:
a. a 5′ external duplex forming region,
b. a 5′ external spacer,
c. a 3′ group I intron fragment,
d. a 5′ internal spacer optionally comprising a 5′ internal duplex forming region,
e. an IRES,
f. an expression sequence,
g. a 3′ internal spacer optionally comprising a 3′ internal duplex forming region,
h. a 5′ group I intron fragment,
i. a 3′ external spacer, and
j. a 3′ external duplex forming region.

55. The pharmaceutical composition of any one of claims 1-51, wherein the circular RNA polynucleotide is made via circularization of a RNA polynucleotide comprising, in the following order:
a. a 3′ group I intron fragment,
b. a 5′ internal spacer comprising a 5′ internal duplex forming region,
c. an IRES,
d. an expression sequence,
e. a 3′ internal spacer comprising a 3′ internal duplex forming region, and
f. a 5′ group I intron fragment.

56. The pharmaceutical composition of any one of claims 1-51, wherein the circular RNA polynucleotide is made via circularization of a RNA polynucleotide comprising, in the following order:
a. a 5′ external duplex forming region,
b. a 5′ external spacer,
c. a 3′ group I intron fragment,
d. a 5′ internal spacer comprising a 5′ internal duplex forming region,
e. an IRES,
f. an expression sequence,
g. a 3′ internal spacer comprising a 3′ internal duplex forming region,
h. a 5′ group I intron fragment,
i. a 3′ external spacer, and
j. a 3′ external duplex forming region.

57. The pharmaceutical composition of any one of claims 1-51, wherein the circular RNA polynucleotide is made via circularization of a RNA polynucleotide comprising, in the following order:
a. a first polyA sequence,
b. a 5′ external duplex forming region,
c. a 5′ external spacer,
d. a 3′ group I intron fragment,
e. a 5′ internal spacer comprising a 5′ internal duplex forming region,
f. an IRES,
g. an expression sequence,
h. a 3′ internal spacer comprising a 3′ internal duplex forming region,
i. a 5′ group I intron fragment,
j. a 3′ external spacer,
k. a 3′ external duplex forming region, and
l. a second polyA sequence.

58. The pharmaceutical composition of any one of claims 1-51, wherein the circular RNA polynucleotide is made via circularization of a RNA polynucleotide comprising, in the following order:
a. a first polyA sequence,
b. a 5′ external spacer,
c. a 3′ group I intron fragment,
d. a 5′ internal spacer comprising a 5′ internal duplex forming region,
e. an IRES,
f. an expression sequence,
g. a 3′ internal spacer comprising a 3′ internal duplex forming region,
h. a 5′ group I intron fragment,
i. a 3′ external spacer, and
j. a second polyA sequence.

59. The pharmaceutical composition of any one of claims 1-51, wherein the circular RNA polynucleotide is made via circularization of a RNA polynucleotide comprising, in the following order:
a. a first polyA sequence,
b. a 5′ external spacer,
c. a 3′ group I intron fragment,
d. a 5′ internal spacer comprising a 5′ internal duplex forming region,
e. an IRES,
f. an expression sequence,
g. a stop condon cassette,
h. a 3′ internal spacer comprising a 3′ internal duplex forming region,
i. a 5′ group I intron fragment,
j. a 3′ external spacer, and
k. a second polyA sequence.

60. The pharmaceutical composition of any one of claims 53-59, wherein at least one of the 3′ or 5′ internal or external spacers has a length of about 8 to about 60 nucleotides.

61. The pharmaceutical composition of any one of claims 53-54 and 56-57, wherein the 3′ and 5′ external duplex forming regions each has a length of about 10-50 nucleotides.

62. The pharmaceutical composition of any one of claims 53-61, wherein the 3′ and 5′ internal duplex forming regions each has a length of about 6-30 nucleotides.

63. The pharmaceutical composition of any one of claims 52-62, wherein the IRES is selected from Table 17, or is a functional fragment or variant thereof.

64. The pharmaceutical composition of any one of claims 52-62, wherein the IRES has a sequence of an IRES from Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, Foot and mouth disease virus, Human enterovirus 71, Equine rhinitis virus, Ectropis obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human eIF4G, Mouse NDST4L, Human LEF1, Mouse HIF1 alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SH1, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picorna-like Virus, CRPV, Apodemus Agrarius Picornavirus, Caprine Kobuvirus, Parabovirus, Salivirus A BNS, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24, or an aptamer to eIF4G.

65. The pharmaceutical composition of any one of claims 57-64, wherein the first and second polyA sequences each have a length of about 15-50 nt.

66. The pharmaceutical composition of any one of claims 57-64, wherein the first and second polyA sequences each have a length of about 20-25 nt.

67. The pharmaceutical composition of any one of claims 1-66, wherein the circular RNA polynucleotide contains at least about 80%, at least about 90%, at least about 95%, or at least about 99% naturally occurring nucleotides.

68. The pharmaceutical composition of any one of claims 1-67, wherein the circular RNA polynucleotide consists of naturally occurring nucleotides.

69. The pharmaceutical composition of any one of claims 33-68, wherein the expression sequence is codon optimized.

70. The pharmaceutical composition of any one of claims 1-69, wherein the circular RNA polynucleotide is optimized to lack at least one microRNA binding site present in an equivalent pre-optimized polynucleotide.

71. The pharmaceutical composition of any one of claims 1-70, wherein the circular RNA polynucleotide is optimized to lack at least one microRNA binding site capable of binding to a microRNA present in a cell within which the circular RNA polynucleotide is expressed.

72. The pharmaceutical composition of any one of claims 1-71, wherein the circular RNA polynucleotide is optimized to lack at least one endonuclease susceptible site present in an equivalent pre-optimized polynucleotide.

73. The pharmaceutical composition of any one of claims 1-72, wherein the circular RNA polynucleotide is optimized to lack at least one endonuclease susceptible site capable of being cleaved by an endonuclease present in a cell within which the endonuclease is expressed.

74. The pharmaceutical composition of any one of claims 1-73, wherein the circular RNA polynucleotide is optimized to lack at least one RNA editing susceptible site present in an equivalent pre-optimized polynucleotide.

75. The pharmaceutical composition of any one of claims 1-74, wherein the circular RNA polynucleotide is from about 100 nt to about 10,000 nt in length.

76. The pharmaceutical composition of any one of claims 1-75, wherein the circular RNA polynucleotide is from about 100 nt to about 15,000 nt in length.

77. The pharmaceutical composition of any one of claims 1-76, wherein the circular RNA is more compact than a reference linear RNA polynucleotide having the same expression sequence as the circular RNA polynucleotide.

78. The pharmaceutical composition of any one of claims 1-77, wherein the composition has a duration of therapeutic effect in a human cell greater than or equal to that of a composition comprising a reference linear RNA polynucleotide having the same expression sequence as the circular RNA polynucleotide.

79. The pharmaceutical composition of claim 78, wherein the reference linear RNA polynucleotide is a linear, unmodified or nucleoside-modified, fully-processed mRNA comprising a cap1 structure and a polyA tail at least 80 nt in length.

80. The pharmaceutical composition of any one of claims 1-79, wherein the composition has a duration of therapeutic effect in vivo in humans greater than that of a composition comprising a reference linear RNA polynucleotide having the same expression sequence as the circular RNA polynucleotide.

81. The pharmaceutical composition of any one of claims 1-80, wherein the composition has an duration of therapeutic effect in vivo in humans of at least about 10, at least about 20, at least about 30, at least about 40, at least about 50, at least about 60, at least about 70, at least about 80, at least about 90, or at least about 100 hours.

82. The pharmaceutical composition of any one of claims 1-81, wherein the composition has a functional half-life in a human cell greater than or equal to that of a pre-determined threshold value.

83. The pharmaceutical composition of any one of claims 1-82, wherein the composition has a functional half-life in vivo in humans greater than that of a pre-determined threshold value.

84. The pharmaceutical composition of claim 82 or 83, wherein the functional half-life is determined by a functional protein assay.

85. The pharmaceutical composition of claim 84, wherein the functional protein assay is an in vitro luciferase assay.

86. The pharmaceutical composition of claim 84, wherein the functional protein assay comprises measuring levels of protein encoded by the expression sequence of the circular RNA polynucleotide in a patient serum or tissue sample.

87. The pharmaceutical composition of any one of claims 82-86, wherein the pre-determined threshold value is the functional half-life of a reference linear RNA polynucleotide comprising the same expression sequence as the circular RNA polynucleotide.

88. The pharmaceutical composition of any one of claims 1-87, wherein the composition has a functional half-life of at least about 20 hours.

89. The pharmaceutic composition of any one of claims 1-88, further comprising a structural lipid and a PEG-modified lipid.

90. The pharmaceutical composition of claim 89, wherein the structural lipid binds to C1q and/or promotes the binding of the transfer vehicle comprising said lipid to C1q compared to a control transfer vehicle lacking the structural lipid and/or increases uptake of C1q-bound transfer vehicle into an immune cell compared to a control transfer vehicle lacking the structural lipid.

91. The pharmaceutical composition of claim 90, wherein the immune cell is a T cell, an NK cell, an NKT cell, a macrophage, or a neutrophil.

92. The pharmaceutical composition of any one of claims 89-91, wherein the structural lipid is cholesterol.

93. The pharmaceutical composition of claim 92, wherein the structural lipid is beta-sitosterol.

94. The pharmaceutical composition of claim 92, wherein the structural lipid is not beta-sitosterol.

95. The pharmaceutical composition of any one of claims 89-94, wherein the PEG-modified lipid is DSPE-PEG, DMG-PEG, or PEG-1.

96. The pharmaceutical composition of claim 95, wherein the PEG-modified lipid is DSPE-PEG(2000).

97. The pharmaceutical composition of any one of claims 1-96, further comprising a helper lipid.

98. The pharmaceutical composition of claim 97, wherein the helper lipid is DSPC or DOPE.

99. The pharmaceutical composition of any one of claims 1-97, further comprising DOPE, cholesterol, and DSPE-PEG.

100. The pharmaceutical composition of any one of claims 1-99, wherein the transfer vehicle comprises about 0.5% to about 4% PEG-modified lipids by molar ratio.

101. The pharmaceutical composition of any one of claims 1-100, wherein the transfer vehicle comprises about 1% to about 2% PEG-modified lipids by molar ratio.

102. The pharmaceutical composition of any one of claims 1-101, wherein the transfer vehicle comprises
a. an ionizable lipid selected from 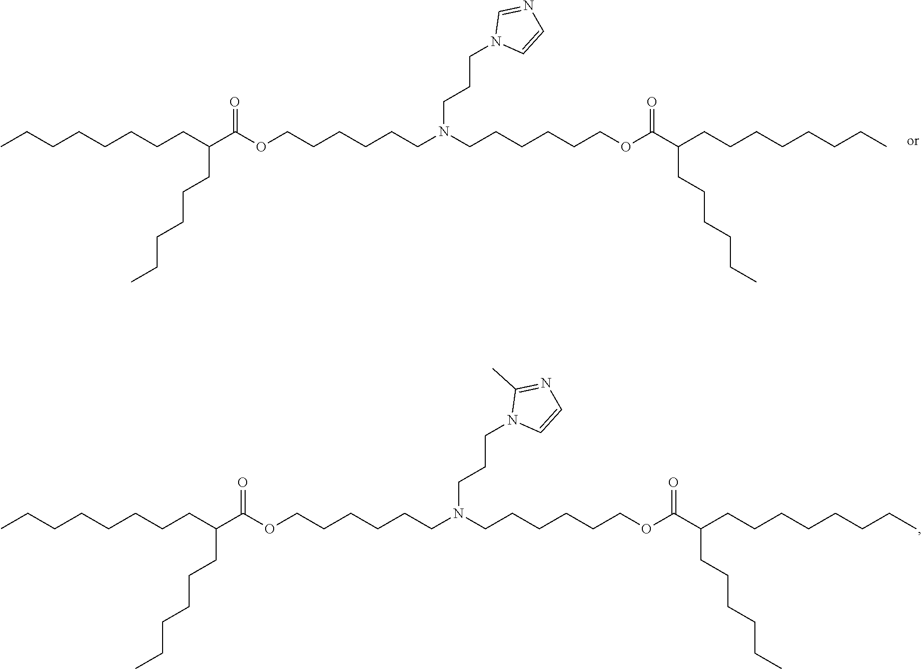
or a mixture thereof,
b. a helper lipid selected from DOPE or DSPC,
c. cholesterol, and
d. a PEG-lipid selected from DSPE-PEG(2000) or DMG-PEG(2000).

103. The pharmaceutical composition of any one of claims 1-101, wherein the transfer vehicle comprises
a. an ionizable lipid selected from 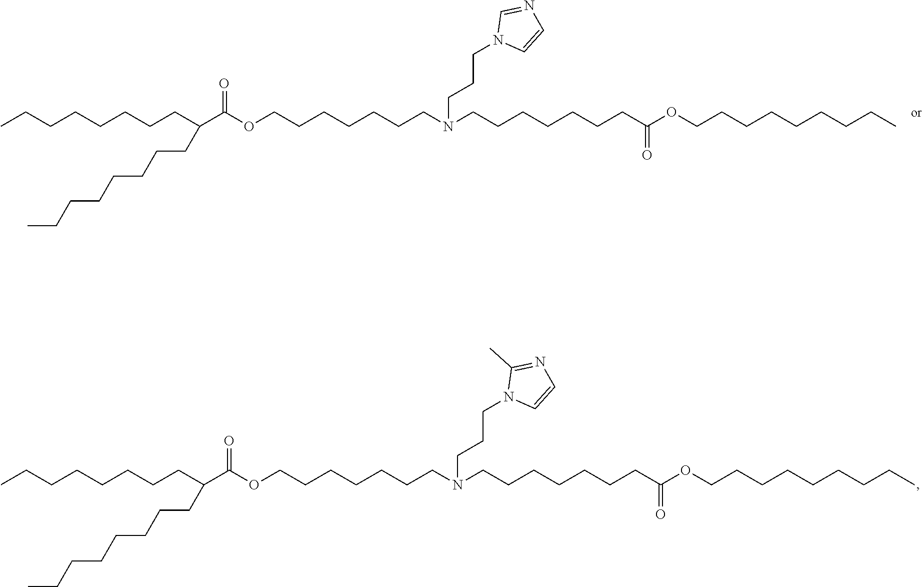
or a mixture thereof,
b. a helper lipid selected from DOPE or DSPC,
c. cholesterol, and
d. a PEG-lipid selected from DSPE-PEG(2000) or DMG-PEG(2000).

104. The pharmaceutical composition of any one of claims 1-101, wherein the transfer vehicle comprises
a. an ionizable lipid selected from 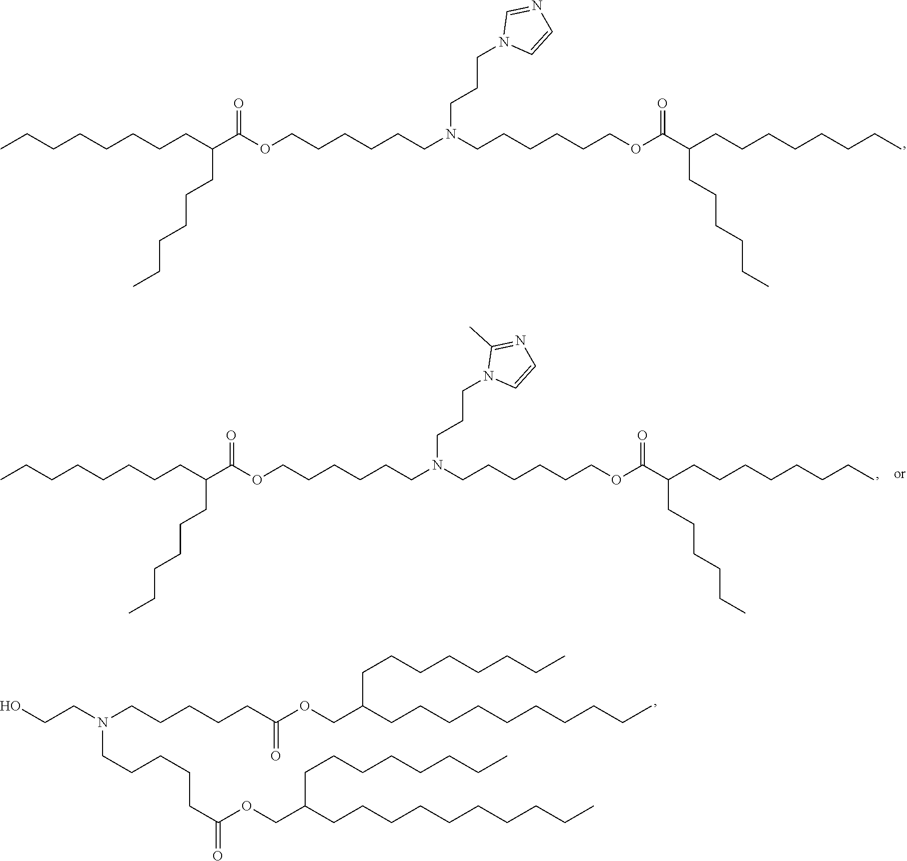
or a mixture thereof,
b. a helper lipid selected from DOPE or DSPC,
c. cholesterol, and
d. a PEH-lipid of DMG-PEG(2000).

105. The pharmaceutical composition of any one of claims 1-101, wherein the transfer vehicle comprises:
a. an ionizable lipid selected from 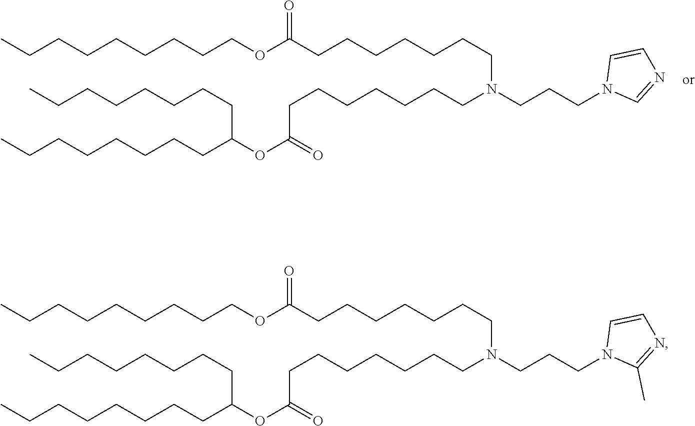 [SMILES: CCCCCCCCCOC(=O)CCCCCCCN(CCCCCCCC(=O)OC(CCCCCCCC)CCCCCCCC)CCCn1ccnc1 ; CCCCCCCCCOC(=O)CCCCCCCN(CCCCCCCC(=O)OC(CCCCCCCC)CCCCCCCC)CCCn1ccnc1C]
or a mixture thereof,
b. a helper lipid selected from DOPE or DSPC,
c. cholesterol, and
d. a PEG-lipid selected from DSPE-PEG(2000), DMG-PEG(2000), or C₁₄-PEG(2000).

106. The pharmaceutical composition of any one of claims 1-101, wherein the transfer vehicle comprises:
a. an ionizable lipid selected from 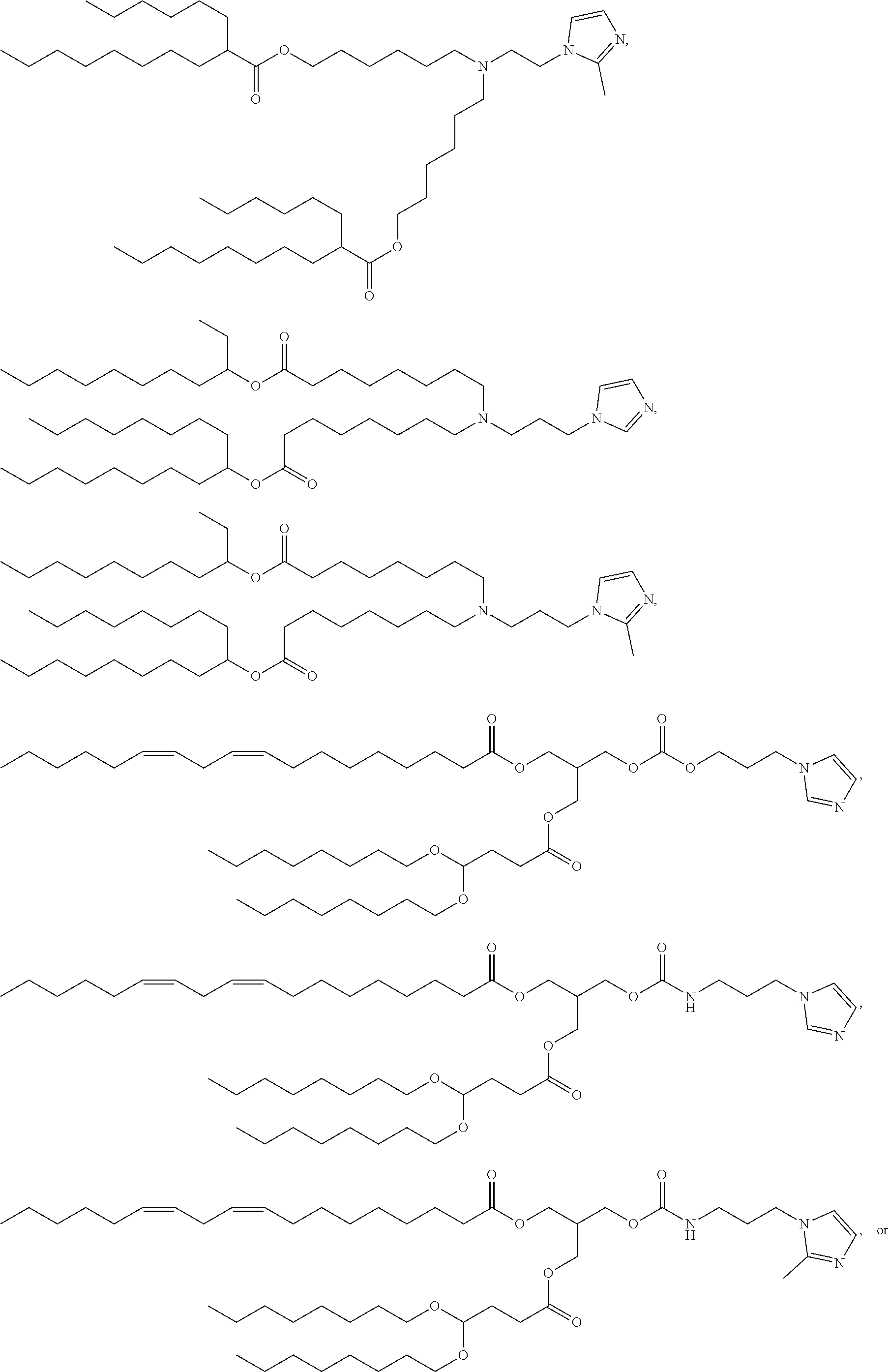 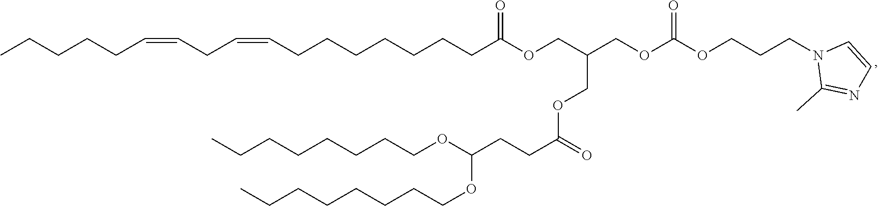 [SMILES: CCCCC/C=C\C/C=C\CCCCCCCC(=O)OCC(COC(=O)CCC(OCCCCCCCC)OCCCCCCCC)COC(=O)OCCCn1ccnc1C]
or a mixture thereof,
b. a helper lipid selected from DOPE or DSPC,
c. cholesterol, and
d. a PEH-lipid of DMG-PEG(2000).

107. The pharmaceutical composition of any one of claims 1-101, wherein the transfer vehicle comprises:
a. an ionizable lipid selected from 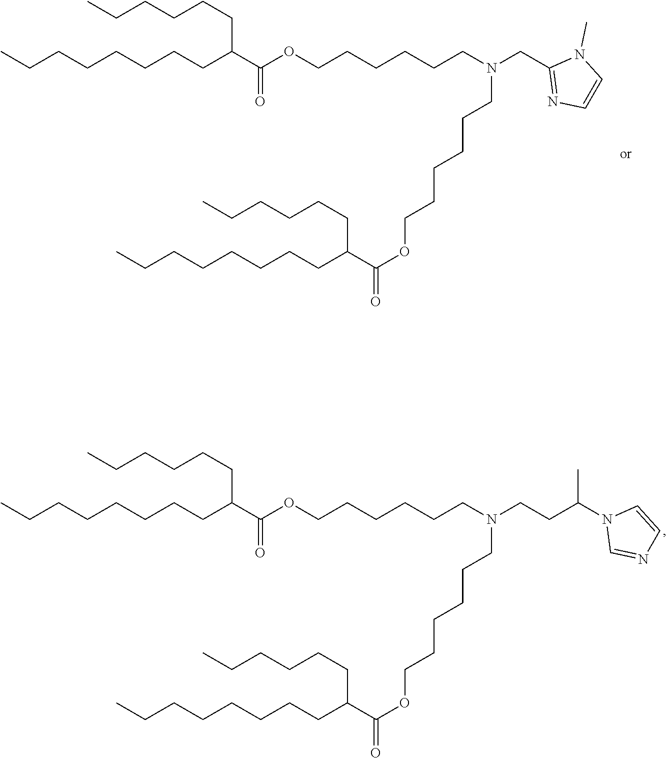 [SMILES: CCCCCCCCC(CCCCCC)C(=O)OCCCCCCN(CCCCCCOC(=O)C(CCCCCC)CCCCCCCC)Cc1nccn1C ; CCCCCCCCC(CCCCCC)C(=O)OCCCCCCN(CCCCCCOC(=O)C(CCCCCC)CCCCCCCC)CCC(C)n1ccnc1]
or a mixture thereof,
b. a helper lipid selected from DOPE or DSPC,
c. cholesterol, and
d. a PEH-lipid selected from DSPE-PEG(2000) or DMG-PEG(2000).

108. The pharmaceutical composition of any one of claims 1-101, wherein the transfer vehicle comprises:
a. an ionizable lipid selected from 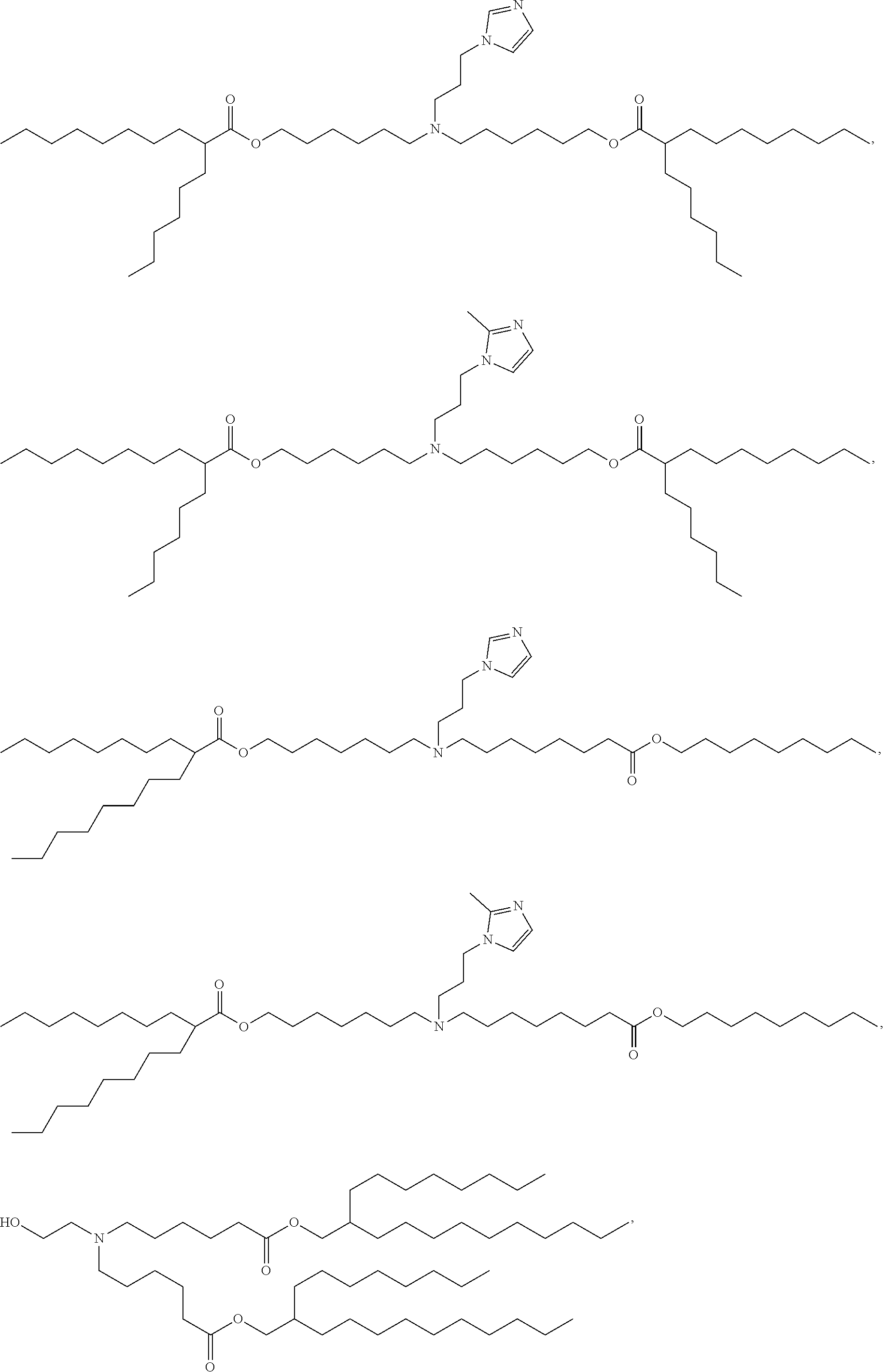 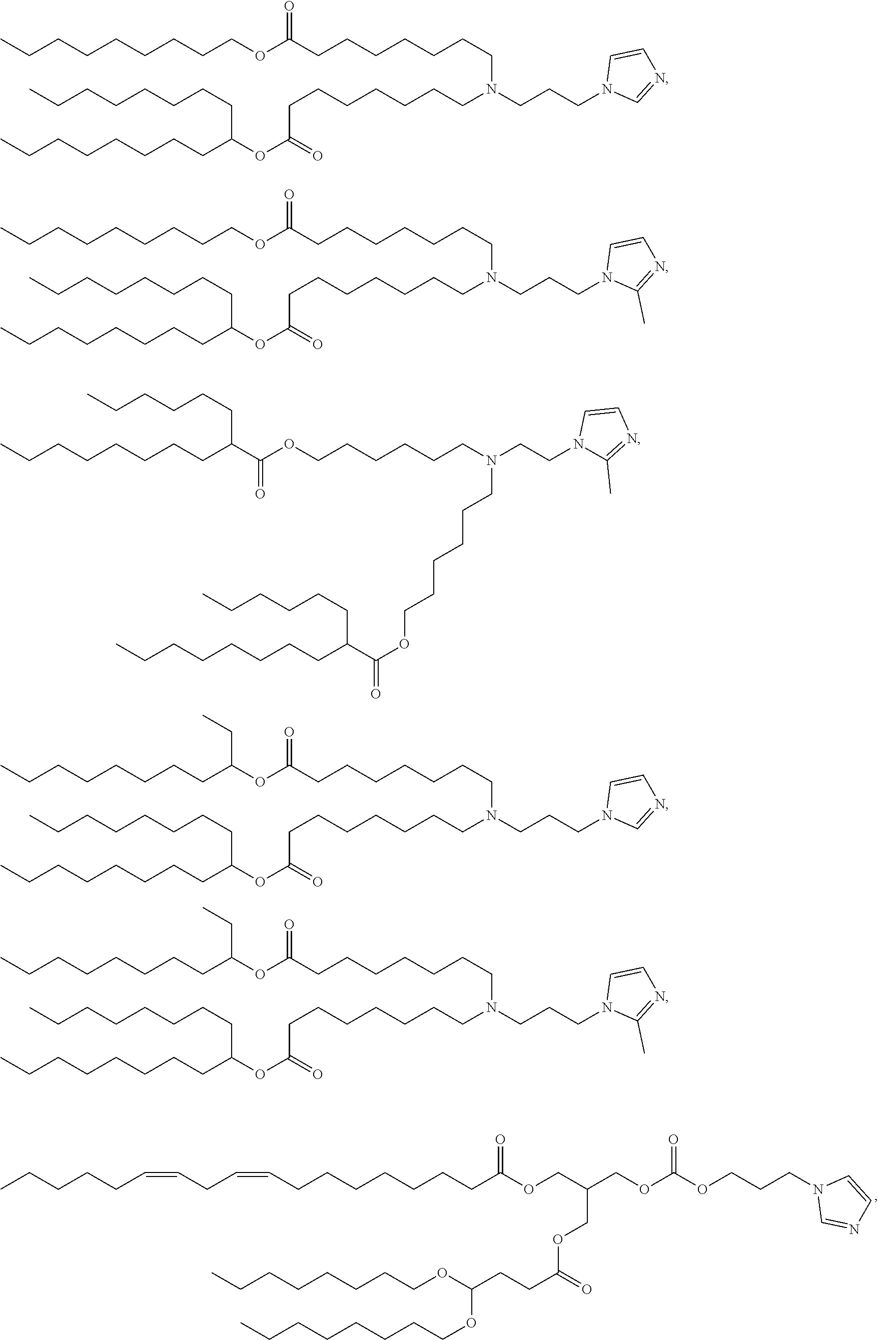 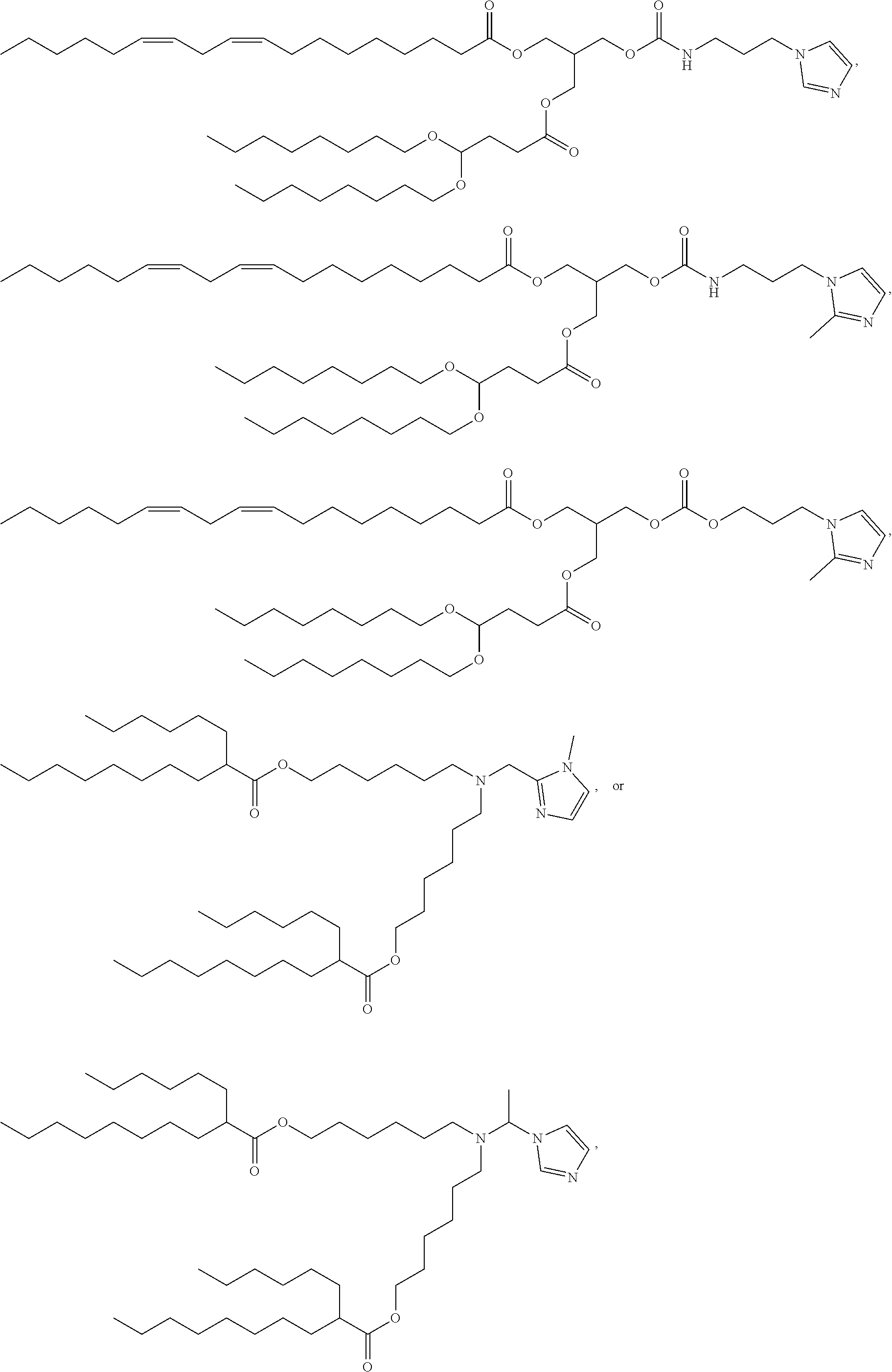 [SMILES: CCCCC/C=C\C/C=C\CCCCCCCC(=O)OCC(COC(=O)CCC(OCCCCCCCC)OCCCCCCCC)COC(=O)NCCCn1ccnc1 ; CCCCC/C=C\C/C=C\CCCCCCCC(=O)OCC(COC(=O)CCC(OCCCCCCCC)OCCCCCCCC)COC(=O)NCCCn1ccnc1C ; CCCCC/C=C\C/C=C\CCCCCCCC(=O)OCC(COC(=O)CCC(OCCCCCCCC)OCCCCCCCC)COC(=O)OCCCn1ccnc1C ; CCCCCCCCC(CCCCCC)C(=O)OCCCCCCN(CCCCCCOC(=O)C(CCCCCC)CCCCCCCC)Cc1nccn1C ; CCCCCCCCC(CCCCCC)C(=O)OCCCCCCN(CCCCCCOC(=O)C(CCCCCC)CCCCCCCC)C(C)n1ccnc1]
or a mixture thereof,
b. a helper lipid selected from DOPE or DSPC,
c. cholesterol, and
d. a PEH-lipid selected from DSPE-PEG(2000), DMG-PEG(2000), or C₁₄-PEG(2000).

109. The pharmaceutical composition of any one of claims 102-108, wherein the molar ratio of ionizable lipid:helper lipid:cholesterol:PEG-lipid is 62:4:33:1.

110. The pharmaceutical composition of any one of claims 102-108, wherein the molar ratio of ionizable lipid:helper lipid:cholesterol:PEG-lipid is 50:10:38.5:1.5.

111. The pharmaceutical composition of any one of claims 102-108, wherein the molar ratio of ionizable lipid:helper lipid:cholesterol:PEG-lipid is 35:16:46.2.5.

112. The pharmaceutical composition of any one of claims 102-108, wherein the molar ratio of ionizable lipid:helper lipid:cholesterol:PEG-lipid is 40:10:40:10.

113. The pharmaceutical composition of any one of claims 102-108, wherein the transfer vehicle comprises the helper lipid of DOPE and the PEG-lipid of DMG-PEG(2000), and wherein the molar ratio of ionizable lipid:DOPE:cholesterol:DMG-PEG(2000) is 62:4:33:1.

114. The pharmaceutical composition of any one of claims 102-108, wherein the transfer vehicle comprises the helper lipid of DOPE and the PEG-lipid of DMG-PEG(2000), and wherein the molar ratio of ionizable lipid:DOPE:cholesterol:DMG-PEG(2000) is 50:10:38.5:1.5.

115. The pharmaceutical composition of any one of claims 102-108, wherein the transfer vehicle comprises the helper lipid of DOPE and the PEG-lipid of DSPE-PEG(2000), and wherein the molar ratio of ionizable lipid:DOPE:cholesterol:DSPE-PEG(2000) is 62:4:33:1.

116. The pharmaceutical composition of any one of claims 102-108, wherein the transfer vehicle comprises the helper lipid of DOPE and the PEG-lipid of DSPE-PEG(2000), and wherein the molar ratio of ionizable lipid:DOPE:cholesterol:DSPE-PEG(2000) is 50:10:38.5:1.5.

117. The pharmaceutical composition of any one of claims 102-108, wherein the transfer vehicle comprises the helper lipid of DSPC and the PEG-lipid of DMG-PEG(2000), and wherein the molar ratio of ionizable lipid:DSPC:cholesterol:DMG-PEG(2000) is 62:4:33:1.

118. The pharmaceutical composition of any one of claims 102-108, wherein the transfer vehicle comprises the helper lipid of DSPC and the PEG-lipid of DMG-PEG(2000), and wherein the molar ratio of ionizable lipid:DSPC:cholesterol:DMG-PEG(2000) is 50:10:38.5:1.5.

119. The pharmaceutical composition of any one of claims 102-108, wherein the transfer vehicle comprises the helper lipid of DSPC and the PEG-lipid of DSPE-PEG(2000), and wherein the molar ratio of ionizable lipid:DSPC:cholesterol:DSPE-PEG(2000) is 62:4:33:1.

120. The pharmaceutical composition of any one of claims 102-108, wherein the transfer vehicle comprises the helper lipid of DSPC and the PEG-lipid of DSPE-PEG(2000), and wherein the molar ratio of ionizable lipid:DSPC:cholesterol:DSPE-PEG(2000) is 50:10:38.5:1.5.

121. The pharmaceutical composition of any one of claims 102-108, wherein the transfer vehicle comprises the helper lipid of DOPE and the PEG-lipid is C₁₄-PEG(2000), and wherein the molar ratio of ionizable lipid:DOPE:cholesterol:C₁₄-PEG(2000) is 35:16:46.5:2.5.

122. The pharmaceutical composition of any one of claims 102-108, wherein the transfer vehicle comprises the helper lipid of DSPC and the PEG-lipid is C14-PEG(2000), and wherein the molar ratio of ionizable lipid:DSPC:cholesterol:C₁₄-PEG(2000) is 35:16:46.5:2.5.

123. The pharmaceutical composition of any one of claims 102-108, wherein the transfer vehicle comprises the helper lipid of DOPE and the PEG-lipid of DMG-PEG(2000), wherein the molar ratio of ionizable lipid:DOPE:cholesterol:DMG-PEG(2000) is 40:10:40:10.

124. The pharmaceutical composition of any one of claims 102-108, wherein the transfer vehicle comprises the helper lipid of DSPC and the PEG-lipid of DMG-PEG(2000), wherein the molar ratio of ionizable lipid:DSPC:cholesterol:DMG-PEG(2000) is 40:10:40:10.

125. The pharmaceutical composition of any one of claims 1-124, having a lipid-nitrogen-to-phosphate (N:P) ratio of about 3 to about 6.

126. The pharmaceutical composition of any one of claims 1-125, having a lipid-nitrogen-to-phosphate (N:P) ratio of about 4, about 4.5, about 5, or about 5.5.

127. The pharmaceutical composition of any one of claims 1-126, wherein the transfer vehicle is formulated for endosomal release of the circular RNA polynucleotide.

128. The pharmaceutical composition of any one of claims 1-127, wherein the transfer vehicle is capable of binding to APOE.

129. The pharmaceutical composition of any one of claims 1-128, wherein the transfer vehicle interacts with apolipoprotein E (APOE) less than an equivalent transfer vehicle loaded with a reference linear RNA having the same expression sequence as the circular RNA polynucleotide.

130. The pharmaceutical composition of any one of claims 1-129, wherein the exterior surface of the transfer vehicle is substantially free of APOE binding sites.

131. The pharmaceutical composition of any one of claims 1-130, wherein the transfer vehicle has a diameter of less than about 120 nm.

132. The pharmaceutical composition of any one of claims 1-131, wherein the transfer vehicle does not form aggregates with a diameter of more than 300 nm.

133. The pharmaceutical composition of any one of claims 1-132, wherein the transfer vehicle has an in vivo half-life of less than about 30 hours.

134. The pharmaceutical composition of any one of claims 1-133, wherein the transfer vehicle is capable of low density lipoprotein receptor (LDLR) dependent uptake into a cell.

135. The pharmaceutical composition of any one of claims 1-134, wherein the transfer vehicle is capable of LDLR independent uptake into a cell.

136. The pharmaceutical composition of any one of claims 1-135, wherein the pharmaceutical composition is substantially free of linear RNA.

137. The pharmaceutical composition of any one of claims 1-136, further comprising a targeting moiety operably connected to the transfer vehicle.

138. The pharmaceutical composition of claim 137, wherein the targeting moiety specifically binds an immune cell antigen or indirectly.

139. The pharmaceutical composition of claim 138, wherein the immune cell antigen is a T cell antigen.

140. The pharmaceutical composition of claim 139, wherein the T cell antigen is selected from the group consisting of CD2, CD3, CD5, CD7, CD8, CD4, beta7 integrin, beta2 integrin, and C1qR.

141. The pharmaceutical composition of claim 137, further comprising an adapter molecule comprising a transfer vehicle binding moiety and a cell binding moiety, wherein the targeting moiety specifically binds the transfer vehicle binding moiety and the cell binding moiety specifically binds a target cell antigen.

142. The pharmaceutical composition of claim 141, wherein the target cell antigen is an immune cell antigen.

143. The pharmaceutical composition of claim 142, wherein the immune cell antigen is a T cell antigen, an NK cell, an NKT cell, a macrophage, or a neutrophil.

144. The pharmaceutical composition of claim 143, wherein the T cell antigen is selected from the group consisting of CD2, CD3, CD5, CD7, CD8, CD4, beta7 integrin, beta2 integrin, CD25, CD39, CD73, A2a Receptor, A2b Receptor, and C1qR.

145. The pharmaceutical composition of claim 138, wherein the immune cell antigen is a macrophage antigen.

146. The pharmaceutical composition of claim 145, wherein the macrophage antigen is selected from the group consisting of mannose receptor, CD206, and C1q.

147. The pharmaceutical composition of any one of claims 137-146, wherein the targeting moiety is a small molecule.

148. The pharmaceutical composition of claim 147, wherein the small molecule is mannose, a lectin, acivicin, biotin, or digoxigenin.

149. The pharmaceutical composition of claim 147, wherein the small molecule binds to an ectoenzyme on an immune cell, wherein the ectoenzyme is selected from the group consisting of CD38, CD73, adenosine 2a receptor, and adenosine 2b receptor.

150. The pharmaceutical composition of any one of claims 137-146, wherein the targeting moiety is a single chain Fv (scFv) fragment, nanobody, peptide, peptide-based macrocycle, minibody, small molecule ligand such as folate, arginylglycylaspartic acid (RGD), or phenol-soluble modulin alpha 1 peptide (PSMA1), heavy chain variable region, light chain variable region or fragment thereof.

151. The pharmaceutical composition of any one of claims 1-150, wherein the ionizable lipid has a half-life in a cell membrane less than about 2 weeks.

152. The pharmaceutical composition of any one of claims 1-151, wherein the ionizable lipid has a half-life in a cell membrane less than about 1 week.

153. The pharmaceutical composition of any one of claims 1-152, wherein the ionizable lipid has a half-life in a cell membrane less than about 30 hours.

154. The pharmaceutical composition of any one of claims 1-153, wherein the ionizable lipid has a half-life in a cell membrane less than the functional half-life of the circular RNA polynucleotide.

155. A method of treating or preventing a disease, disorder, or condition, comprising administering an effective amount of a pharmaceutical composition of any one of claims 1-154.

156. The method of claim 155, wherein the disease, disorder, or condition is associated with aberrant expression, activity, or localization of a polypeptide selected from Tables 27 or 28.

157. The method of claim 155 or 156, wherein the circular RNA polynucleotide encodes a therapeutic protein.

158. The method of claim 157, wherein therapeutic protein expression in the spleen is higher than therapeutic protein expression in the liver.

159. The method of claim 158, wherein therapeutic protein expression in the spleen is at least about 2.9x therapeutic protein expression in the liver.

160. The method of claim 158, wherein the therapeutic protein is not expressed at functional levels in the liver.

161. The method of claim 158, wherein the therapeutic protein is not expressed at detectable levels in the liver.

162. The method of claim 158, wherein therapeutic protein expression in the spleen is at least about 50% of total therapeutic protein expression.

163. The method of claim 158, wherein therapeutic protein expression in the spleen is at least about 63% of total therapeutic protein expression.

164. A linear RNA polynucleotide comprising, from 5′ to 3′, a 3′ group I intron fragment, an Internal Ribosome Entry Site (IRES), an expression sequence, and a 5′ group I intron fragment, further comprising a first spacer 5′ to the 3′ group I intron fragment and/or a second spacer 3′ to the 5′ group I intron fragment.

165. The linear RNA polynucleotide of claim 164, comprising first spacer 5′ to the 3′ group I intron fragment.

166. The linear RNA polynucleotide of claim 165, wherein the first spacer has a length of 10-50 nucleotides, optionally 10-20 nucleotides, further optionally about 15 nucleotides.

167. The linear RNA polynucleotide of claim 165 or 166, wherein the first spacer comprises a polyA sequence.

168. The linear RNA polynucleotide of any one of claims 164-167, comprising a second spacer 3′ to the 5′ group I intron fragment.

169. The linear RNA polynucleotide of claim 168, wherein the second spacer has a length of 10-50 nucleotides, optionally 10-20 nucleotides, further optionally about 15 nucleotides.

170. The linear RNA polynucleotide of claim 168 or 169, wherein the second spacer comprises a polyA sequence.

171. The linear RNA polynucleotide of any one of claims 164-170, further comprising a third spacer between the 3′ group I intron fragment and the Internal Ribosome Entry Site (IRES).

172. The linear RNA polynucleotide of claim 171, wherein the third spacer has a length of about 10 to about 60 nucleotides.

173. The linear RNA polynucleotide of any of claims 164-172, further comprising a first and a second duplex forming regions capable of forming a duplex.

174. The linear RNA polynucleotide of claim 173, wherein the first and second duplex forming regions each have a length of about 9 to 19 nucleotides, optionally wherein the first and second duplex forming regions each have a length of about 30 nucleotides.

175. The linear RNA polynucleotide of any of claims 164-174, comprising, from 5′ to 3′, a first polyA sequence, a 5′ external spacer, a 3′ group I intron fragment, a 5′ internal spacer comprising a 5′ internal duplex forming region, an IRES, an expression sequence, a stop condon cassette, a 3′ internal spacer comprising a 3′ internal duplex forming region, a 5′ group I intron fragment, a 3′ external spacer, and a second polyA sequence.

176. The linear RNA polynucleotide of any of claims 164-175, wherein the linear RNA polynucleotide has enhanced expression, circularization efficiency, functional stability, and/or stability as compared to a reference linear RNA polynucleotide, wherein the reference linear RNA polynucleotide comprises, from 5′ to 3′, a reference 3′ group I intron fragment, a reference IRES, a reference expression sequence, and a reference 5′ group I intron fragment, and does not comprise a spacer 5′ to the 3′ group I intron fragment or a spacer 3′ to the 5′ group I intron fragment.

177. The linear RNA polynucleotide of claim 176, wherein the expression sequence and the reference expression sequence have the same sequence.

178. The linear RNA polynucleotide of claim 176 or 177, wherein the IRES and the reference IRES have the same sequence.

179. The linear RNA polynucleotide of any of claims 164-178, wherein the linear RNA polynucleotide comprises a 3′ Anabaena group I intron fragment and a 5′ Anabaena group I intron fragment.

180. The linear RNA polynucleotide of claim 179, wherein the reference RNA polynucleotide comprises a reference 3′ Anabaena group I intron fragment and a reference 5′ Anabaena group I intron fragment.

181. The linear RNA polynucleotide of claim 180, wherein the reference 3′ Anabaena group I intron fragment and reference 5′ Anabaena group I intron fragment were generated using the L₆-5 permutation site.

182. The linear RNA polynucleotide of claim 180 or 181, wherein the 3′ Anabaena group I intron fragment and 5′ Anabaena group I intron fragment were not generated using the L₆-5 permutation site.

183. The linear RNA polynucleotide of any of claims 179-182, wherein the 3′ Anabaena group I intron fragment comprises or consists of a sequence selected from SEQ ID NO: 112-123 and 125-150.

184. The linear RNA polynucleotide of claim 183, wherein the 5′ Anabaena group I intron fragment comprises a corresponding sequence selected from SEQ ID NO: 73-84 and 86-111.

185. The linear RNA polynucleotide of any of claims 180-184, wherein the 5′ Anabaena group I intron fragment comprises or consists of a sequence selected from SEQ ID NO: 73-84 and 86-111.

186. The linear RNA polynucleotide of claim 185, wherein the 3′ Anabaena group I intron fragment comprises or consists of a corresponding sequence selected from SEQ ID NO: 112-124 and 125-150.

187. The linear RNA polynucleotide of any one of claims 164-186, wherein the IRES comprises a nucleotide sequence selected from SEQ ID NOs: 348-351.

188. The linear RNA polynucleotide of any of claims 164-186, wherein the reference IRES is CVB3.

189. The linear RNA polynucleotide of any of claims 164-186, wherein the IRES is not CVB3.

190. The linear RNA polynucleotide of any of claims 164-186, wherein the IRES comprises a sequence selected from SEQ ID NOs: 1-64 and 66-72.

191. A circular RNA polynucleotide produced from the linear RNA of any one of claims 164-190.

192. A circular RNA polynucleotide comprising, from 5′ to 3′, a 3′ group I intron fragment, an IRES, an expression sequence, and a 5′ group I intron fragment, wherein the IRES comprises a nucleotide sequence selected from SEQ ID NOs: 348-351.

193. The circular RNA polynucleotide of claim 192, further comprising a spacer between the 3′ group I intron fragment and the IRES.

194. The circular RNA polynucleotide of claim 192 or 193, further comprising a first and a second duplex forming regions capable of forming a duplex.

195. The circular RNA polynucleotide of claim 194, wherein the first and second duplex forming regions each have a length of about 9 to 19 nucleotides.

196. The circular RNA polynucleotide of claim 194, wherein the first and second duplex forming regions each have a length of about 30 nucleotides.

197. The RNA polynucleotide of any one of claims 164-196, wherein the expression sequence has a size of at least about 1,000 nt, at least about 2,000 nt, at least about 3,000 nt, at least about 4,000 nt, or at least about 5,000 nt.

198. The RNA polynucleotide of any one of claims 164-197, comprises natural nucleotides.

199. The RNA polynucleotide of any one of claims 164-198, wherein the expression sequence is codon optimized.

200. The RNA polynucleotide of any one of claims 164-199, further comprising a translation termination cassette comprising at least one stop codon in each reading frame.

201. The RNA polynucleotide of claim 200, wherein the translation termination cassette comprises at least two stop codons in the reading frame of the expression sequence.

202. The RNA polynucleotide of any one of claims 164-201, optimized to lack at least one microRNA binding site present in an equivalent pre-optimized polynucleotide.

203. The RNA polynucleotide of any one of claims 164-202, optimized to lack at least one endonuclease susceptible site present in an equivalent pre-optimized polynucleotide.

204. The RNA polynucleotide of any one of claims 164-203, optimized to lack at least one RNA editing susceptible site present in an equivalent pre-optimized polynucleotide.

205. The RNA polynucleotide of any one of claims 164-204, comprising at least 2 expression sequences.

206. The RNA polynucleotide of claim 205, wherein each expression sequence encodes a different therapeutic protein.

207. The circular RNA polynucleotide of any one of claims 191-206, wherein the circular RNA polynucleotide is from about 100 to 15,000 nucleotides, optionally about 100 to 12,000 nucleotides, further optionally about 100 to 10,000 nucleotides in length.

208. The circular RNA polynucleotide of any one of claims 191-207, having an in vivo duration of therapeutic effect in humans of at least about 20 hours.

209. The circular RNA polynucleotide of any one of claims 191-208, having a functional half-life of at least about 20 hours.

210. The circular RNA polynucleotide of any one of claims 191-209, having a duration of therapeutic effect in a human cell greater than or equal to that of an equivalent linear RNA polynucleotide comprising the same expression sequence.

211. The circular RNA polynucleotide of any one of claims 191-210, having a functional half-life in a human cell greater than or equal to that of an equivalent linear RNA polynucleotide comprising the same expression sequence.

212. The circular RNA polynucleotide of any one of claims 191-211, having an in vivo duration of therapeutic effect in humans greater than that of an equivalent linear RNA polynucleotide having the same expression sequence.

213. The circular RNA polynucleotide of any one of claims 191-212, having an in vivo functional half-life in humans greater than that of an equivalent linear RNA polynucleotide having the same expression sequence.

214. A pharmaceutical composition comprising a circular RNA polynucleotide of any one of claims 191-213, a nanoparticle, and optionally, a targeting moiety operably connected to the nanoparticle.

215. The pharmaceutical composition of claim 214, wherein the nanoparticle is a lipid nanoparticle, a core-shell nanoparticle, a biodegradable nanoparticle, a biodegradable lipid nanoparticle, a polymer nanoparticle, or a biodegradable polymer nanoparticle.

216. The pharmaceutical composition of claim 214 or 215, comprising a targeting moiety, wherein the targeting moiety mediates receptor-mediated endocytosis or direct fusion selectively into cells of a selected cell population or tissue in the absence of cell isolation or purification.

217. The pharmaceutical composition of any one of claims 214-216, wherein the targeting moiety is a scfv, nanobody, peptide, minibody, polynucleotide aptamer, heavy chain variable region, light chain variable region or fragment thereof.

218. The pharmaceutical composition of any one of claims 214-217, wherein less than 1%, by weight, of the polynucleotides in the composition are double stranded RNA, DNA splints, or triphosphorylated RNA.

219. The pharmaceutical composition of any one of claims 214-218, wherein less than 1%, by weight, of the polynucleotides and proteins in the pharmaceutical composition are double stranded RNA, DNA splints, triphosphorylated RNA, phosphatase proteins, protein ligases, and capping enzymes.

220. A method of treating a subject in need thereof comprising administering a therapeutically effective amount of a composition comprising the circular RNA polynucleotide of any one of claims 191-213, a nanoparticle, and optionally, a targeting moiety operably connected to the nanoparticle.

221. A method of treating a subject in need thereof comprising administering a therapeutically effective amount of the pharmaceutical composition of any one of claims 214-219.

222. The method of claim 220 or 221, wherein the targeting moiety is an scfv, nanobody, peptide, minibody, heavy chain variable region, light chain variable region, an extracellular domain of a TCR, or a fragment thereof.

223. The method of any one of claims 220-222, wherein the nanoparticle is a lipid nanoparticle, a core-shell nanoparticle, or a biodegradable nanoparticle.

224. The method of any one of claims 220-223, wherein the nanoparticle comprises one or more cationic lipids, ionizable lipids, or poly (3-amino esters.

225. The method of any one of claims 220-224, wherein the nanoparticle comprises one or more non-cationic lipids.

226. The method of any one of claims 220-225, wherein the nanoparticle comprises one or more PEG-modified lipids, polyglutamic acid lipids, or Hyaluronic acid lipids.

227. The method of any one of claims 220-226, wherein the nanoparticle comprises cholesterol.

228. The method of any one of claims 220-227, wherein the nanoparticle comprises arachidonic acid or oleic acid.

229. The method of any one of claims 220-228, wherein the composition comprises a targeting moiety, wherein the targeting moiety mediates receptor-mediated endocytosis selectively into cells of a selected cell population in the absence of cell selection or purification.

230. The method of any one of claims 220-229, wherein the nanoparticle comprises more than one circular RNA polynucleotide.

231. A DNA vector encoding the RNA polynucleotide of any one of claims 164-213.

232. The DNA vector of claim 231, further comprising a transcription regulatory sequence.

233. The DNA vector of claim 232, wherein the transcription regulatory sequence comprises a promoter and/or an enhancer.

234. The DNA vector of claim 233, wherein the promoter comprises a T7 promoter.

235. The DNA vector of any one of claims 231-234, wherein the DNA vector comprises a circular DNA.

236. The DNA vector of any one of claims 231-235, wherein the DNA vector comprises a linear DNA.

237. A prokaryotic cell comprising the DNA vector according to any one of claims 231-236.

238. A eukaryotic cell comprising the circular RNA polynucleotide according to any one of claims 191-213.

239. The eukaryotic cell of claim 238, wherein the eukaryotic cell is a human cell.

240. A method of producing a circular RNA polynucleotide, the method comprising incubating the linear RNA polynucleotide of any one of claims 164-190 and 197-206 under suitable conditions for circularization.

241. The method of producing a circular RNA polynucleotide, the method comprising incubating the DNA of any one of claims 231-236 under suitable conditions for transcription.

242. The method of claim 241, wherein the DNA is transcribed in vitro.

243. The method of claim 241, wherein the suitable conditions comprises adenosine triphosphate (ATP), guanine triphosphate (GTP), cytosine triphosphate (CTP), uridine triphosphate (UTP), and an RNA polymerase.

244. The method of claim 241, wherein the suitable conditions further comprises guanine monophosphate (GMP).

245. The method of claim 244, wherein the ratio of GMP concentration to GTP concentration is within the range of about 3:1 to about 15:1, optionally about 4:1, 5:1, or 6:1.

246. A method of producing a circular RNA polynucleotide, the method comprising culturing the prokaryotic cell of claim 237 under suitable conditions for transcribing the DNA in the cell.

247. The method of any one of claims 240-246, further comprising purifying a circular RNA polynucleotide.

248. The method of claim 247, wherein the circular RNA polynucleotide is purified by negative selection using an affinity oligonucleotide that hybridizes with the first or second spacer conjugated to a solid surface.

249. The method of claim 248, wherein the first or second spacer comprises a polyA sequence, and wherein the affinity oligonucleotide is a deoxythymine oligonucleotide.

250. The pharmaceutical composition of any one of claims 1-154 and 214-219, wherein the pharmaceutical composition:liver cell ratio by weight is no more than 1:5.

251. The pharmaceutical composition of any one of claims 1-154 and 214-219, wherein the pharmaceutical composition:spleen cell ratio by weight is no more than 7:10.

252. The method of any one of claims 155-163 and 221-230, wherein the pharmaceutical composition is administered to the subject in need with 0.5 mg per 1 kg of body mass at day 0, 2, 5, 7, and 9 intervals.


SEQUENCE LISTING (717 sequences)

SEQ ID NO: 1 - DNA, 585 bp, Unknown
  Description of Unknown Encephalomyocarditis virus sequence
cccccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 2 - DNA, 568 bp, Unknown
  Description of Unknown Encephalomyocarditis virus sequence
ctccccctccccccccttactatactggccgaagccacttggaataaggccggtgtgcgtttgtctacatgctattttctaccgcattaccgtcttatggtaatgtgagggtccagaacctgaccctgtcttcttgacgaacactcctaggggtctttcccctctcgacaaaggagtgtaaggtctgttgaatgtcgtgaaggaagcagttcctctggaagcttcttaaagacaaacaacgtctgtagcgaccctttgcaggcagcggaaccccccacctggtgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacgtgctttacacgtgttgagtcgaggtgaaaaaacgtctaggccccccgaaccacggggacgtggttttcctttgaaaaccacgattacaat

SEQ ID NO: 3 - DNA, 696 bp, Unknown
  Description of Unknown Encephalomyocarditis virus sequence
ttgccagtctgctcgatatcgcaggctgggtccgtgactacccactccccctttcaacgtgaaggctacgatagtgccagggcgggtactgccgtaagtgccaccccaaacaacaacaacaaaacaaactccccctccccccccttactatactggccgaagccacttggaataaggccggtgtgcgtttgtctacatgctattttctaccgcattaccgtcttatggtaatgtgagggtccagaacctgaccctgtcttcttgacgaacactcctaggggtctttcccctctcgacaaaggagtgtaaggtctgttgaatgtcgtgaaggaagcagttcctctggaagcttcttaaagacaaacaacgtctgtagcgaccctttgcaggcagcggaaccccccacctggtgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacgtgctttacacgtgttgagtcgaggtgaaaaaacgtctaggccccccgaaccacggggacgtggttttcctttgaaaaccacgattacaat

SEQ ID NO: 4 - DNA, 697 bp, Unknown
  Description of Unknown Encephalomyocarditis virus sequence
ttgccagtctgctcgatatcgcaggctgggtccgtgactacccactccccctttcaacgtgaaggctacgatagtgccagggcgggtactgccgtaagtgccaccccaaaacaacaacaaccccccctctccctcctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 5 - DNA, 562 bp, Unknown
  Description of Unknown Encephalomyocarditis virus sequence
ccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 6 - DNA, 156 bp, Unknown
  Description of Unknown Picobirnavirus sp. sequence
gtaaattaaatgctatttacaaaatttaaacagaaaggagagatgttatgaaccggttttacaaggtttcatacatcgaaaatagcactacctggggcagccgacacactaacatcgtctgtttaaccagaagtgttactgaaaggaggttattta

SEQ ID NO: 7 - DNA, 340 bp, Unknown
  Description of Unknown Hepatitis C virus sequence
acctgcccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggacatttgggcgtgcccccgcaagactgctagccgagtagcgttgggttgcgaaaggccttgtggtactgcctgatagggtgcttgcgagtgccccgggaggtctcgtagaccgtgcatc

SEQ ID NO: 8 - DNA, 315 bp, Unknown
  Description of Unknown Human Cosavirus sequence
ctacaagctttgtgtaaacaaacttttgtttggcttttctcaagcttctctcacatcaggccccaaagatgtcctgaaggtaccccgtgtatctgaggatgagcaccatcgactacccggacctgcaaaattttgcaaacgcatgtggtatcccagccccctcctctcggggagggggctttgctcactcagcacaggatctgatcaggagatccacctccggtgctttacaccggggcgtggatttaaaaattgcccaaggcctggcgcacaacctaggggactaggttttccttatattttaaagctgtcaat

SEQ ID NO: 9 - DNA, 182 bp, Unknown
  Description of Unknown Human Cosavirus sequence
gtcttaggacgacgcatgtggtatcccagcccccgcctacattggcgggggcttttgaagcaccagacactggatctgatcaggaggagggtagctgctttacagcccctcttaaaaattgcccaaggtccggccacccaacctaggggactaggttttccttttatttttaaattgtcatt

SEQ ID NO: 10 - DNA, 759 bp, Unknown
  Description of Unknown Human Cosavirus sequence
acatgggggagactgcatgtggcagtcttgaaacgtgtggtttgacgtctaccttatatggcagtgggtggagtactgcaaagatgtcaccgtgctttacacggtttttgaaccccacaccggctgtttgacgctcgtagggcagcaggtttattttcattaaaattcttactttctagctgcatgagttctattcatgcagacggagtgatactcccgttccttcttggacaggttgcctccacgccctttgtggatcttaaggtgaccaagtcactggtgttggaggtgaagatagagagtcctcttgggaatgtcatgtggctgtgccaggggttgtagcgatgccattcgtgtgtgcggatttcctctcgtggtgacacgagcctcacaggccaaaagccccgtccgaaaggacccgaatggtggagtgaccctgactcccccctgcatagttttgtgattaggaacttgaggaatttctgtcataaatctctatcacatcaggccccaaagatgtcctgaaggtaccctgtgtatctgaggatgagcaccaccgactacccggacttgcattagcagacacatgtggttgcccagccccacctcttcagaggtggggctttgctcactcagcacaggatctgatcaggagccccgctcgtgtgctttacactcgacgcggggttaaaaattgcccaaggcctggcacaacaacctaggggactaggttttcctatttttgtaaattatgtcaat

SEQ ID NO: 11 - DNA, 478 bp, Unknown
  Description of Unknown Human rhinovirus sequence
gtgacaatcagccagattgttaacggtcaagcacttctgtttccccggtacccttgtatacgcttcacccgaggcgaaaagtgaggttatcgttatccgcaaagtgcctacgagaagcctagtagcacttttgaagcctatggctggtcgctcaactgtttacccagcagtagacctggcagatgaggctagatgttccccaccagcgatggtgatctagcctgcgtggctgcctgcacactctattgagtgtgaagccagaaagtggacaaggtgtgaagagcctattgtgctcactttgagtcctccggcccctgaatgtggctaatcctaaccccgtagctgttgcatgtaatccaacatgtctgcagtcgtaatgggcaactatgggatggaaccaactactttgggtgtccgtgtttcttgtttttctttatgcttgcttatggtgacaactgtagttattacatttgttacc

SEQ ID NO: 12 - DNA, 628 bp, Unknown
  Description of Unknown Human rhinovirus sequence
ttaaaacagcggatgggtatcccaccattcgacccattgggtgtagtactctggtactatgtacctttgtacgcctgtttctccccaaccacccttccttaaaattcccacccatgaaacgttagaagcttgacattaaagtacaataggtggcgccatatccaatggtgtctatgtacaagcacttctgtttcccaggagcgaggtataggctgtacccactgccaaaagcctttaaccgttatccgccaaccaactacgtaacagttagtaccatcttgttcttgactggacgttcgatcaggtggattttccctccactagtttggtcgatgaggctaggaattccccacgggtgaccgtgtcctagcctgcgtggcggccaacccagcttatgctgggacgcccttttaaggacatggtgtgaagactcgcatgtgcttggttgtgagtcctccggcccctgaatgcggctaaccttaaccctagagccttatgccacgatccagtggttgtaaggtcgtaatgagcaattccgggacgggaccgactactttgggtgtccgtgtttctcatttttcttcatattgtcttatggtcacagcatatatatacatatactgtgatc

SEQ ID NO: 13 - DNA, 618 bp, Unknown
  Description of Unknown Human rhinovirus sequence
ttaaaactgggagtgggttgttcccactcactccacccatgcggtgttgtactctgttattacggtaactttgtacgccagtttttcccacccttccccataatgtaacttagaagtttgtacaatatgaccaataggtgacaatcatccagactgtcaaaggtcaagcacttctgtttccccggtcaatgaggatatgctttacccaaggcaaaaaccttagagatcgttatccccacactgcctacacagagcccagtaccatttttgatataattgggttggtcgctccctgcaaacccagcagtagacctggcagatgaggctggacattccccactggcgacagtggtccagcctgcgtggctgcctgctcacccttcttgggtgagaagcctaattattgacaaggtgtgaagagccgcgtgtgctcagtgtgcttcctccggcccctgaatgtggctaaccttaaccctgcagccgttgcccataatccaatgggtttgcggtcgtaatgcgtaagtgcgggatgggaccaactactttgggtgtccgtgtttcctgtttttcttttgattgcattttatggtgacaatttatagtgtatagattgtcatc

SEQ ID NO: 14 - DNA, 610 bp, Unknown
  Description of Unknown Human rhinovirus sequence
ttaaaactgggtacaggttgttcccacctgtatcacccacgtggtgtggtgctcttgtattccggtacacttgcacgccagtttgccacccctcacccgtcgtaacttagaagctaacaactcgaccaacaggcggtggtaaaccataccacttacggtcaagcactcctgtttccccggtatgcgaggaatagactcctacagggttgaagcctcaagtatcgttatccgcattggtactacgcaaagcttagtagtgccttgaaagtcccttggttggtcgctccgctagtttcccctagtagacctggcagatgaggcaggacactccccactggcgacagtggtcctgcctgcgtggctgcctgcgcacccttaggggtgcgaagccaagtgacagacaaggtgtgaagagccccgtgtgctaccaatgagtcctccggcccctgaatgcggctaatccaaccccacagctattgcacacaagccagtgtgtatgtagtcgtaatgagcaattgtgggacggaaccgactactttgggtgtccgtgtttccttttattcttatcattctgcttatggtgacaatactgtgaaatagtgttgttacc

SEQ ID NO: 15 - DNA, 614 bp, Unknown
  Description of Unknown Human rhinovirus sequence
taaaactggatccaggttgttcccacctggatctcctattgggagttgtactctattattccggtaattttgtacgccagttttatcttccccctccccaattgtaacttagaaggttatcaatacgaccaataggtggtagttagccaaactaccaaaggtcaagcacttctgtttccccggtcaaagttgatatgctccaacagggcaaaaacaactgagatcgttatccgcaaagtgcctacgcaaagcctagtaacacctttgaagatttatggttggtcgttccgctatttcccatagtagacctggcagatgaggctagaaatcccccactggcgacagtgctctagcctgcgtggctgcctgcgcaccccttgggtgcgaagccatacattggacaaggtgtgaagagccccgtgtgctcactttgagtcctccggcccctgaatgtggctaaccttaaccctgcagctagtgcatgtaatccaacatgttgctagtcgtaatgagtaattgcgggacgggaccaactactttgggtgtccgtgtttcactttttccttttaatattgcttatggtgacaatatatatagctatatatattgacacc

SEQ ID NO: 16 - DNA, 624 bp, Unknown
  Description of Unknown Salivirus A sequence
ttcccctgcaaccattacgcttactcgcatgtgcattgagtggtgcatgtgttgaacaaacagctacactcacatgggggcgggttttcccgccctacggcttctcgcgaggcccacccctcccctttctcccataactacagtgctttggtaggtaagcatcctgatcccccgcggaagctgctcacgtggcaactgtggggacccagacaggttatcaaaggcacccggtctttccgccttcaggagtatccctgctagcgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactcaggggatacctcaaggcacccaggaatgccagggaggtaccccgcttcacagcgggatctgaccctggggtaaatgtctgcggggggtcttcttggcccacttctcagtacttttcagg

SEQ ID NO: 17 - DNA, 730 bp, Unknown
  Description of Unknown Salivirus FHB sequence
acatggggggtctgcggacggcttcggcccacccgcgacaagaatgccgtcatctgtcctcattacccgtattccttcccttcccccgcaaccaccacgcttactcgcgcacgtgttgagtggcacgtgcgttgtccaaacagctacacccacacccttcggggcgggtttgtcccgccctcgggttcctcgcggaacccccccctccctctctctctttctatccgccctcacttcccataactacagtgctttggtaggtgagcaccctgaccccccgcggaagctgctaacgtggcaactgtggggatccaggcaggttatcaaaggcacccggtctttccgccttcaggagtatctctgccggtgaattccggtagggctctgcttggtgccaacctcccccaaatgcgcgctgcgggagtgctcttccccaactcatcttagtaacctctcatgtgtgtgcttggtcagcatatctgaggcgacgttccgctgtcccagaccagtccagcaatggacgggccagtgtgcgtagtcgctttccggttttccggcgcatgtttggcgaaacgctgaggtaaggttggtgtgcccaacgcccgtaatttggtgatacctcaagaccacccaggaatgccagggaggtaccccacttcggtgggatctgaccctgggctaattgtctacggtggttcttcttgcttccacttctcttttttctggcatg

SEQ ID NO: 18 - DNA, 709 bp, Unknown
  Description of Unknown Salivirus NG-J1 sequence
tatggcaggcgggcttgtggacggcttcggcccacccacagcaagaatgccatcatctgtcctcacccccaattttcccttttcttcccctgcaaccattacgcttactcgcatgtgcattgagtggtgcatgtgttgaacaaacagctacactcacatgggggcgggttttcccgccctacggcctctcgcgaggcccaccccttccctccccttataactacagtgctttggtaggtaagcatcctgatcccccgcggaagctgctcacgtggcaactgtggggacccagacaggttatcaaaggcacccggtctttccgccttcaggagtatccctactagtgaattctagcggggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgcgtgtagtcgtcttccggcttgtccggggcatgtttggtgaaccggtggggtaaggttggtgtgcccaacgcccgtactttggtgacacctcaagaccacccaggaatgccagggaggtaccccacctcacggtgggatctgaccctgggctaattgtctacggtggttcttcttgcttccacttctttcttctgttcacg

SEQ ID NO: 19 - DNA, 708 bp, Unknown
  Description of Unknown Human Parechovirus sequence
tttgaaaggggtctcctagagagcttggccgtcgggccttataccccgacttgctgagtttctctaggagagcccttttcccagccctgaggcggctggtcaataaaagcctcaaacgtaactaacacctaagaagatcatgtaaaccctatgcctggtctccactattcgaaggcaacttgcaataagaagagtgggatcaagacgcttaaagcatagagacagttttcttttctaacccacatttgtgtggggtggcagatggcgtgccataactctaatagtgagataccacgcttgtggaccttatgctcacacagccatcctctagtaagtttgtgagacgtctggtgacgtgtgggaacttattggaaacaacattttgctgcaaagcatcctactgccagcggaaaaacacctggtaacaggtgcctctggggccaaaagccaaggtttaacagaccctttaggattggttctaaacctgagatgttgtggaagatatttagtacctgctgatctggtagttatgcaaacactagttgtaaggcccatgaaggatgcccagaaggtacccgtaggtaacaagtgacactatggatctgatttggggccagatacctctatcttggtgatctggttaaaaaacatctaatgggccaaacccgggggggatccccggtttcctcttattctatcaatgccact

SEQ ID NO: 20 - DNA, 508 bp, Unknown
  Description of Unknown Crohivirus B sequence
gtataagagacaggtgtttgccttgtcttcggactggcatcttgggaccaaccccccttttccccagccatgggttaaatggcaataaaggacgtaacaactttgtaaccattaagctttgtaattttgtaaccactaagctttgtgcacataatgtaaccatcaagcttgttagtcccagcaggaggtttgcatgcttgtagccgaaatggggctcgaccccccatagtaggatacttgattttgcattccattgtggacctgcaaactctacacatagaggctttgtcttgcatctaaacacctgagtacagtgtgtacctagaccctatagtacgggaggaccgtttgtttcctcaataaccctacataataggctaggtgggcatgcccaatttgcaagatcccagactgggggtcggtctgggcagggttagatccctgttagctactgcctgatagggtggtgctcaaccatgtgtagtttaaattgagctgttcatatacc

SEQ ID NO: 21 - DNA, 248 bp, Unknown
  Description of Unknown Picornaviridae sp. sequence
actgaagatcctacagtaactactgccccaatgaacgccacagatgggtctgctgatgactacctatcttagtgctagttgaggtttgaagtgagccggtttttagaagaaccagtttctgaacattatcatccccagcatctattctatacgcacaagatagatagtcatcagcagacacatctgtgctactgcttgatagagttgcggctggtcaacttagattggtataaccagttgagtggcaa

SEQ ID NO: 22 - DNA, 516 bp, Unknown
  Description of Unknown Rosavirus M-7 sequence
tatgcatcactggacggcctaacctcggtcgtggcttcttgccgatttcagcgctaccaggctttctggtctcgccaggcgttgattagtaggtgcactgtctaagtgaagacagcagtgctctctgtgaaaagttgatgacactcttcaggtttgtagcgatcactcaaggctagcggatttccccgtgtggtaacacacgcctctaggcccagaaggcacggtgttgacagcaccccttgagtggctggtcttccccaccagcacctgatttgtggattcttcctagtaacggacaagcatggctgctcttaagcattcagtgcgtccggggctgaaggatgcccagaaggtacccgcaggtaacgataagctcactgtggatctgatctggggctgcgggctgggtgtctttccacccagccaaaacccgtaaaacggtagtcgcagttaaaaaacgtctaggccccacccccccagggatggggggttcccttaaaccctcacaagttcaac

SEQ ID NO: 23 - DNA, 478 bp, Unknown
  Description of Unknown Shanbavirus A sequence
tgaaaagggggcgcagggtggtggtggttactaaatacccaccatcgccctgcacttcccttttcccctgtggctcagggtcacttagccccctctttgggttaccagtagttttctacccctgggcacagggttaactatgcaagacggaacaacaatctcttagtccccctcgccgatagtgggctcgacccccatgtgtaggagtggataagggacggagtgagccgatacggggaagagtgtgcggtcacaccttaattccatgagcgctgcgaagaaggaagctgtgaacaatggcgacctgaaccgtacacatggagctccacaggcatggtactcgttagactacgcagcctggttgggagtgggtataccctgggtgagccgccagtgaatgggagttcactggttaacacacactgcctgatagggtcagggcctcctgtccccgccgtaatgaggtagaccatatgcc

SEQ ID NO: 24 - DNA, 403 bp, Unknown
  Description of Unknown Pasivirus A sequence
gcggctggatattctggccgtgcaactgcttttgaccagtggctctgggtaacttagccaaagtgtccttctccctttccctattatatgttttatggctttgtctggtcttgtttagtttatatataagatcctttccgccgatatagacctcgacagtctagtgtaggaggattggtgatattaatttgccccagaagagtgaccgtgacacatagaaaccatgagtacatgtgtatccgtggaggatcgcccgggactggattccatatcccattgccatcccaacaagcggagggtatacccactatgtgcacgtctgcagtgggagtctgcagatttagtcatactgcctgatagggtgtgggcctgcactctggggtactcaggctgtttatataat

SEQ ID NO: 25 - DNA, 404 bp, Unknown
  Description of Unknown Pasivirus A sequence
gctggactttctggctgcgcaactgcttttaaccagtggctctgggttacttagccaaaaccccctttccccgtaccctagtttgtgtgtgtattattattttgttgttgttttgtaaatttttatataagatcctttccgccgatatagacctcgacagtctagtgtaggaggattggtgatattaatatgccccagaagagtgaccgtgacacatagaaaccatgagtacatgtgtatccgtggaggatcgcccgggactggattccatatcccattgccatcccaacaaacggagggtatacccgctatgtgcgcgtctacagtgggaatctgtagatttagtcatactgcctgatagggtgtgggcctgcactctggggtactcaggctgtttatataat

SEQ ID NO: 26 - DNA, 744 bp, Unknown
  Description of Unknown Echovirus E14 sequence
ttaaaacagcctgtgggttgttcccatccacagggcccactgggcgccagcactctggtattgcggtaccttagtgcgcctgttttatatacccgtcccccaaacgtaacttagacgcatgtcaacgaagaccaatagtaagcgcagcacaccagctgtgttccggtcaagcacttctgttaccccggaccgagtatcaataagctactcacgtggctgaaggagaaaacgttcgttacccgaccaattacttcaagaaacctagtaacaccatgaaggttgcgcagtgtttcgctccgcacaaccccagtgtagatcaggtcgatgagtcaccgcattccccacgggtgaccgtggcggtggctgcgctggcggcctgcccatggggaaacccatgggacgcttcaatactgacatggtgcgaagagtctattgagctaattggtagtcctccggcccctgaatgcggctaatcctaactgcggagcagatacccacacaccagtgggcagtctgtcgtaacgggcaactctgcagcggaaccgactactttgggtgtccgtgtttctctttatccttatactggctgcttatggtgacaattgagagattgttaccatatagctattggattggccatccggtgacaaatagagcaattgtgtatttgtttgttggtttcgtgccattaaattacaaggttctaaacacccttaatcttattatagcattcaacacaacaaa

SEQ ID NO: 27 - DNA, 663 bp, Unknown
  Description of Unknown Human Parechovirus sequence
gtacattagatgcgtcatctgcaactttagtcaataaattacctccaatgtcattaccaacattccctaccttttcactaacacctaagacaacaagtacctatgcctggtctccactattcgaaggcaacttgcaataagaagagtggaattaagacgcttaaagcatagagctagttatcttttctaacccacaaagttttgtggggtggcagatggcgtgccataactctattagtgagataccatgcttgtggatcttatgctcacacagccatcctctagtaagttgataaggtgtctggtgatatgtgggaactcacatgaaccattaatttaccgtaaggtatcctatagccagcggaatcacatctggtgacagatgcctctggggccgaaagccaaggtttaacagaccctataggattggtttcaaaacctgaattgatgtggattgtgtatagtacctgttgatctggtaacagtgtcaacactagttgtaaggcccacgaaggatgcccagaaggtacccgtaggtaacaagtgacactatggatctgatctggggccagctacctctatcatggtgagttggttaaaaaacgtctagtgggccaaacccaggggggatccctggtttccttttacctaatcaaagccact

SEQ ID NO: 28 - DNA, 744 bp, Unknown
  Description of Unknown Aichi virus sequence
tttgaaaagggggtgggggggcctcggccccctcaccctcttttccggtggtctggtcccggaccaccgttactccattcagcttcttcggaacctgttcggaggaattaaacgggcacccatactccccccaccccccttttgtaactaagtatgtgtgctcgtgatcttgactcccacggaacggaccgatccgttggtgaacaaacagctaggtccacatcctcccttcccctgggagggcccccgccctcccacatcctccccccagcctgacgtatcacaggctgtgtgaagcccccgcgaaagctgctcacgtggcaattgtgggtccccccttcatcaagacaccaggtctttcctccttaaggctagccccggcgtgtgaattcacgttgggcaactagtggtgtcactgtgcgctcccaatctcggccgcggagtgctgttccccaagccaaacccctggcccttcactatgtgcctggcaagcatatctgagaaggtgttccgctgtggctgccaacctggtgacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccacctccgggtgggatctgagcctgggctaattgtctacgggtagtttcatttccaatccttttatgtcggagtc

SEQ ID NO: 29 - DNA, 734 bp, Unknown
  Description of Unknown Hepatitis A virus sequence
ttcaagaggggtctccggagttttccggaacccctcttggaagtccatggtgaggggacttgatacctcaccgccgtttgcctaggctataggctaaatttccctttccctgtccttcccctatttccttttgttttgtttgtaaatattaattcctgcaggttcagggttctttaatctgtttctctataagaacactcaatttttcacgctttctgtctcctttcttccagggctctccccttgccctaggctctggccgttgcgcccggcggggtcaactccatgattagcatggagctgtaggagtctaaattggggacgcagatgtttgggacgtcgccttgcagtgttaacttggctttcatgaacctctttgatcttccacaaggggtaggctacgggtgaaacctcttaggctaatacttcaatgaagagatgccttggatagggtaacagcggcggatattggtgagttgttaagacaaaaaccattcaacgccggaggactggctctcatccagtggatgcattgagggaattgattgtcagggctgtctctaggtttaatctcagacctctctgtgcttagggcaaacactatttggccttaaatgggatcctgtgagagggggtccctccattgacagctggactgttctttggggccttatgtggtgtttgcctctgaggtactcaggggcatttaggtttttcctcattcttaaataata

SEQ ID NO: 30 - DNA, 638 bp, Unknown
  Description of Unknown Phopivirus sequence
gggagtaaacctcaccaccgtttgccgtggtttacggctacctatttttggatgtaaatattaattcctgcaggttcaggtctcttgaattatgtccacgctagtggcactctcttacccataagtgacgccttagcggaacctttctacacttgatgtggttaggggttacattatttccctgggccttctttggccctttttcccctgcactatcattctttcttccgggctctcagcatgccaatgttccgaccggtgcgcccgccggggttaactccatggttagcatggagctgtaggccctaaaagtgctgacactggaactggactattgaagcatacactgttaactgaaacatgtaactccaatcgatcttctacaaggggtaggctacgggtgaaaccccttaggttaatactcatattgagagatacttctgataggttaaggttgctggataatggtgagtttaacgacaaaaaccattcaacagctgtgggccaacctcatcaggtagatgcttttggagccaagtgcgtaggggtgtgtgtggaaatgcttcagtggaaggtgccctcccgaaaggtcgtaggggtaatcaggggcagttaggtttccacaattacaatttgaa

SEQ ID NO: 31 - DNA, 743 bp, Unknown
  Description of Unknown CVA10 sequence
gctcttccgatctgggttgttcccacccacagggcccactgggcgccagcactctgattccacggaatctttgtgcgcctgttttacaacccttcccaatttgtaacgtagaagcaatacacactactgatcaatagtaggcatggcgcgccagtcatgtcatgatcaagcacttctgttcccccggactgagtatcaatagactgctcacgcggttgaaggagaaaacgttcgttacccggctaactacttcgagaaacctagtagcaccatggaagctgcggagtgtttcgctcagcactttccccgtgtagatcaggtcgatgagtcactgcaatccccacgggcgaccgtggcagtggctgcgttggcggcctgcctatggggcaacccataggacgctctaatgtggacatggtgcgaagagtctattgagctagttagtagtcctccggcccctgaatgcggctaatcctaactgcggagcacatgccttcaacccaggaggtggtgtgtcgtaacgggtaactctgcagcggaaccgactactttgggtgtccgtgtttccttttatccttatattggctgcttatggtgacaatcacggaattgttgccatatagctattggattggccatccggtgtctaacagagctattgtatacctatttgttggatttactcccctatcatacaaatctctgaacactttgtgctttatactgaacttaaacacacgaaa

SEQ ID NO: 32 - DNA, 746 bp, Unknown
  Description of Unknown Enterovirus C sequence
ttaaaacagctctggggttgttcccaccccagaggcccacgtggcggccagtacaccggtaccacggtacccttgtacgcctgttttatactcccctccccgtaaactagaagcacgaaacacaagttcaatagaagggggtacagaccagtaccaccacgaacaagcacttctgttcccccggtgaggtcacatagactgtccccacggtcaaaagtgactgatccgttatccgctcacgtacttcggaaagcctagtaccaccttggaatctacgatgcgttgcgctcagcactcgaccccggagtgtagcttaggctgatgagtctggacgttccccactggtgacagtggtccaggctgcgttggcggcctacctgtggtccaaaaccacaggacgctagtagtgaacaaggtgtgaagagcccactgagctacctgagaatcctccggcccctgaatgcggctaatcccaaccacggagcaggtaatcgcaaaccagcggtcagcctgtcgtaacgcgtaagtctgtggcggaaccgactactttgggtgtccgtgtttccttttatttttatggtggctgcttatggtgacaatcatagattgttatcataaagcaaattggattggccatccggagtgagctaaactatctatttctctgagtgttggattcgtttcacccacattctgaacaatcagcctcattagtgttaccctgttaataagacgatatcatcacg

SEQ ID NO: 33 - DNA, 726 bp, Unknown
  Description of Unknown Enterovirus D sequence
ttaaaacagctctggggttgttcccaccccagaggcccacgtggcggctagtactccggtaccccggtacccttgtacgcctgttttatactccctttcccaagtaactttagaagaaataaactaatgttcaacaggagggggtacaaaccagtaccaccacgaacacacacttctgtttccccggtgaagttgcatagactgtacccacggttgaaagcgatgaatccgttacccgcttaggtacttcgagaagcctagtatcatcttggaatcttcgatgcgttgcgatcagcactctaccccgagtgtagcttgggtcgatgagtctggacaccccacaccggcgacgtggtccaggctgcgttggcggcctacccatggctagcaccatgggacgctagttgtgaacaaggtgcgaagagcctattgagctacctgagagtcctccggcccctgaatgcggctaatcccaaccacggagcaaatgctcacaatccagtgagtggtttgtcgtaatgcgcaagtctgtggcggaaccgactactttgggtgtccgtgtttccttttatttttattatggctgcttatggtgacaatctgagattgttatcatatagctattggattagccatccggtgatatcttgaaattttgccataactttttcacaaatcctacaacattacactacactttctcttgaataattgagacaactcata

SEQ ID NO: 34 - DNA, 737 bp, Unknown
  Description of Unknown Enterovirus J sequence
ttaaaatagcctcagggttgttcccaccctgagggcccacgtggtgtagtactctggtattacggtacctttgtacgcctattttatacccccttccccaagtaatttagaagcaagcacaaaccagttcagtagtaagcagtacaatccagtactgtaatgaacaagtacttctgttaccccggaagggtctatcggtaagctgtacccacggctgaagaatgacctaccgttaaccggctacctacttcgagaagcctagtaatgccgttgaagttttattgacgttacgctcagcacactaccccgtgtgtagttttggctgatgagtcacggcactccccacgggcgaccgtggccgtggctgcgttggcggccaaccaaggagtgcaagctccttggacgtcatattacagacatggtgtgaagagcctattgagctaggtggtagtcctccggcccctgaatgcggctaatcctaactccggagcatatcggtgcgaaccagcacttggtgtgttgtaatacgtaagtctggagcggaaccgactactttgggtgtccgtgtttcctgttttaacttttatggctgcttatggtgacaatttaacattgttaccatatagctgttgggttggccatccggattttgttataaaaccatttcctcgtgccttgacctttaacacatttgtgaacttctttaaatcccttttattagtccttaaatactaaga

SEQ ID NO: 35 - DNA, 327 bp, Pegivirus sp.
aactgttgttgtagcaatgcgcatattgctacttcggtacgcctaattggtaggcgcccggccgaccggccccgcaagggcctagtaggacgtgtgacaatgccatgagggatcatgacactggggtgagcggaggcagcaccgaagtcgggtgaactcgactcccagtgcgaccacctggcttggtcgttcatggagggcatgcccacgggaacgctgatcgtgcaaagggatgggtccctgcactggtgccatgcgcggcaccactccgtacagcctgatagggtggcggcgggcccccccagtgtgacgtccgtggagcgcaac

SEQ ID NO: 36 - DNA, 281 bp, Unknown
  Description of Unknown GB virus C sequence
tgacgtgggggggttgattttccccccccggcactgggtgcaagccccagaaaccgacgcctatctaagtagacgcaatgactcggcgccgactcggcgaccggccaaaaggtggtggatgggtgatgacagggttggtaggtcgtaaatcccggtcatcctggtagccactataggtgggtcttaagagaaggtcaagattcctcttacgcctgcggcgagaccgcgcacggtccacaggtgttggccctaccggtgtgaataagggcccgacatcaggc

SEQ ID NO: 37 - DNA, 459 bp, Unknown
  Description of Unknown GB virus C sequence
gacgtgggggggttgatcccccccctttggcactgggtgcaagccccagaaaccgacgcctatttaaacagacgttaagaaccggcgccgacccggcgaccggccaaaaggtggtggatgggtgatgccagggttggtaggtcgtaaatcccggtcatcttggtagccactataggtgggtcttaagggttggttaaggtccctctggcgcttgtggcgagaaagcgcacggtccacaggtgttggccctaccggtgtgaataagggcccgacgtcaggctcgtcgttaaaccgagcccactacccacctgggcaaacaacgcccacgtacggtccacgtcgcccttcaatgtctctcttgaccaataggcttagccggcgagttgacaaggaccagtgggggctgggcggtaggggaaggacccctgccgctgcccttcccggtggagtgggaaatgc

SEQ ID NO: 38 - DNA, 350 bp, Unknown
  Description of Unknown GB virus C sequence
tgacgtgggggggttgatccgccccccccggcactcggtgcaagccccataaaccgacgcctatctaagtagacgcaatgactcggcgccgactcggcgaccggccaaaaggtggtggatgggtggtgacagggttggtaggtcgtaaatcccggtcatcctggtagccactataggtgggtcttaagagaaggtcaagactcctcttgtgcctgcggcgagaccgcgcacggtccacaggtgctggccctaccggtgtgaataagggcccgacgtcaggctcgtcgttaaaccgagcccgtcacccacctgggcaaacgacgcccacgtacggtccacgtcgcccttca

SEQ ID NO: 39 - DNA, 318 bp, Pegivirus A
tgtagcaatgcgcatattgctacttcggtacgcctaattggtaggcgcccggccgaccggccccgcaagggcctagtaggacgtgtgacaatgccatgcgggatcatgacactggggtgagcggaggcagcaccgaagtcgggtgaactcgactcccagtgcgaccacctggcttggtcgttcatggagggcatgcccacgggaacgctgatcgtgcaaagggatgggtccctgcactggtgccatgcgcggcaccactccgtacagcctgatagggtggcggcgggcccccccagtgtgacgtccgtggagcgcaac

SEQ ID NO: 40 - DNA, 399 bp, Unknown
  Description of Unknown Pasivirus A sequence
attttctggccgtgtagctgcttttgaccagtggctctgggttacttagccaaatcccccttccttcacccttttaaatttgatggtctgtgttgtttgttttgtcttgtctaaataatatataagatccttcccgccgatacagacctcgacagtctggtgtaggagggttggtgttattaatttgccccagaagagtgaccgtgacacatagaaaccatgagtacatgtgtatccgtggaggatcgcccgggactggattccatatcccattgccatcccaacaagcggagggtatacccactatgtgcgcgtttgcagtgggaatctgcaaatttagtcatactgcctgatagggtgtgggcctgcactctggggtactcaggctgttcatataat

SEQ ID NO: 41 - DNA, 430 bp, Sapelovirus sp.
cccctccacccttaaggtggttgtatcccacataccccaccctcccttccaaagtggacggacaactggattttgactaacggcaagtctgaatggtatgatttggatacgtttaaacggcagtagcgtggcgagctatggaaaaatcgcaattgtcgatagccatgttagtgacgcgcttcggcgtgctcctttggtgattcggcgactggttacaggagagtaggcagtgagctatgggcaaacctctacagtattacttagagggaatgtgcaattgagacttgacgagcgtctctttgagatgtggcgcatgctcttggcattaccatagtgagcttccaggttgggaaacctggactgggcctatactacctgatagggtcgcggctggccgcctgtaactagtatagtcagttgaaaccccccc

SEQ ID NO: 42 - DNA, 504 bp, Rosavirus B
gtctctttagtgtctatgcttcagagagcggtgaactgacaccgttgcttcttgcacagcccttcgtgccggtctttccggttctcgacagcgttgggcatcatggctagttaggctaagatagtggatgatctagtgaacagttttggattgtttggagttttgtagcgatgctagtagtgtgtgtggacctccccacgtggtaacacgtgccccacaggccaaaagccaaggtgttgaaagcacccctactagtcccagactcacccatctgggaactcctctcatgaaaaatcttagtaacttttgattcggctattcatcaacctctctagtcaagggctgaaggatgcccggaaggtacccgcaggtaacgataagctcactgtggatctgatccggggctttggtgcgaccgtctgtccggcgtagccagagttaaaaaacgtctaggcccttccaccccaagggattggggtttccccaatcatttgaaagttcact

SEQ ID NO: 43 - DNA, 532 bp, Unknown
  Description of Unknown Bakunsa virus sequence
ttttgaacgccacctcggagcgatatccggggaccccctcccctttttccttcctaccttcttcccaaatttccctcttcccttgttattttggtttggatttcctggacatgactcggacggatctatctcatttgctttgtgtctgctccaccagtggcatggtcgaaagatcatcaacactggacgtgtactgtaatggccaaacgtgcccacaggggaaaccatgccggtcgctgtagcggcgggtggacgtggtggacccctctccctgctcataaactttgggtaggtgaagggttcaagcgacgcttgccgtgagggcgcatccggatggtgggaaccaacaaactaggctgtaatggccgacctcaggtggatgagctagggctgctgcaccaaaagggactcgattcgatatcccggcctggtagcctagtgcagtggactcgtagttgggaatctacgactggcctagtacagggtgatagccccgtttcccacgcccacctgttgtagggacacccccccc

SEQ ID NO: 44 - DNA, 494 bp, Tremovirus A
tttgaaagaggcctccggagtgtccggaggctctctttcgacccaacccatactggggggtgtgtgggaccgtacctggagtgcacggtatatatgcattcccgcatggcaagggcgtgctaccttgccccttgacgcatggtatgcgtcatcatttgccttggttaagccccatagaaacgaggcgtcacgtgccgaaaatccctttgcgtttcacagaaccatcctaaccatgggtgtagtatgggaatcgtgtatggggatgattaggatctctcgtagagggataggtgtgccattcaaatccagggagtactctggctctgacattgggacatttgatgtaaccggacctggttcagtatccgggttgtcctgtattgttacggtgtatccgtcttggcacactgaaagggtatttttgggtaatcctttcctactgcctgatagggtggcgtgcccggccacgagagattaagggtagcaatttaaac

SEQ ID NO: 45 - DNA, 378 bp, Unknown
  Description of Unknown Swine Pasivirus 1 sequence
gcttttgaccagtggctctgggttacttagccaagtccctttctcttattttcactagtttatgttgtgtgttgtctgttttgttttgtttaaattgtatacaagatccttcccgccgacacagacctcgacagtctggtgtaggagggttggtgatattaatttgccccaaaagagtgaccgtgatacgtggaaaccatgagtacatgtgtatccgtggaggatcgcccgggactggattccatatcccattgccatcccaacaaacggagggtatacccaccacgtgcgcgtttgcagtgggaatctgcaaatttagtcatactgcctgatagggtgtgggcctgcactttggggtactcaggctgttcatataat

SEQ ID NO: 46 - DNA, 319 bp, Unknown
  Description of Unknown Parechovirus-like virus PLV-CHN sequence
acatggggtatgttgtctgtcctgttttgttgaaacaatatataagatcctttccgccgatatagacctcgacagtctagtgtaggaggattggtgatagtaacttgccccagaagagtgaccgtgacacatagaaaccatgagtacatgtgtatccgtggaggatcgcccgggactggattccatatcccattgccatcccaacaaacggagggtatacccactatgtgcgcgtttgcagtgggagcctgcaaatttagtcatactgcctgatagggtgtgggcctgcactctggggtactcaggctgtttatataat

SEQ ID NO: 47 - DNA, 420 bp, Unknown
  Description of Unknown Pasivirus A sequence
tgaaaaagtggttgtgcagctggattttccggctgtgcaactgcttttgaccagtggctctgggttacttagccaaattcctttcccttatccctattggtttgtgttgtgtgttgtttgttttgttttgtcttaactatatacaagatccttcccgccgatacagacctcgacagtctggtgtaggagggttggtgttattaatttgccccaaaagagtgaccgtgacacgtggaaaccatgagtacatgtgtatccgtggaggatcgcccgggactggattccatatcccattgccatcccaacaaacggagggtatacccaccacgtgcgcgtttgcagtgggaatctgcaaatttagtcatactgcctgatagggtgtgggcctgcactttggggtactcaggctgtttatataat

SEQ ID NO: 48 - DNA, 373 bp, Sicinivirus sp.
gtgtcattaaggtgtgtttggaagttcgaattagctggtttgtggtgattagtagaccccctggaggtacccaattcggatctgaccagggacccgtgactataccgctccggtaattcgggtttaaaacaatgaacgtcaccacacaattacttttctcattttattttcatcattgtcttcctatttaccgattacactcgatttccttggatgttcctggagatttccctggttacctggaccctcattattgttgttgtttcacccagcgagctgtcccaattgcttattatttgcgcttacaacttcgtcctaatatttttctggttgatcgggttgattgagctcccgggctatcctgccattcaac

SEQ ID NO: 49 - DNA, 280 bp, Hepacivirus K
gggaacaatggtccgtccgcggaacgactctagccatgagtctagtacgagtgcgtgccacccattagcacaaaaaccactgactgagccacacccctcccggaatcctgagtacaggacattcgctcggacgacgcatgagcctccatgccgagaaaattgggtatacccacgggtaaggggtggccacccagcgggaatctgggggctggtcactgactatggtacagcctgatagggtgctgccgcagcgtcagtggtatgcggctgttcatggaac

SEQ ID NO: 50 - DNA, 384 bp, Hepacivirus A
acctccgtgctaggcacggtgcgttgtcagcgttttgcgcttgcatgcgctacacgcgtcgtccaacgcggagggaacttcacatcaccatgtgtcactccccctatggagggttccaccccgcttacacggaaatgggttaaccatacccaaagtacgggtatgcgggtcctcctagggcccccccggcaggtcgagggagctggaattcgtgaattcgtgagtacacgaaaatcgcggcttgaacgtctttgaccttcggagccgaaatttgggcgtgccccacgaaggaaggcgggggcggtgttgggccgccgccccctttatcccacggtctgataggatgcttgcgagggcacctgccggtctcgtagaccataggac

SEQ ID NO: 51 - DNA, 384 bp, Unknown
  Description of Unknown Bovine viral diarrhea virus 1 sequence
gtatacgagaatttgcctaggacctcgtttacaatatgggcaatctaaaattataattaggcctaagggacaaatcctcctcagcgaaggccgaaaagaggctagccatgcccttagtaggactagcaaaataaggggggtagcaacagtggtgagttcgttggatggctgaagccctgagtacagggtagtcgtcagtggttcgacgcttcggaggacaagcctcgagataccacgtggacgagggcatgcccacagcacatcttaacctggacgggggtcgttcaggtgaaaacggtttaaccaaccgctacgaatacagcctgatagggtgctgcagaggcccactgtattgctactgaaaatctctgctgtacatggcac

SEQ ID NO: 52 - DNA, 372 bp, Unknown
  Description of Unknown Border Disease virus sequence
gtatacgggagtagctcatgcccgtatacaaaattggatattccaaaactcgattgggttagggagccctcctagcgacggccgaaccgtgttaaccatacacgtagtaggactagcagacgggaggactagccatcgtggtgagatccctgagcagtctaaatcctgagtacaggatagtcgtcagtagttcaacgcaggcacggttctgccttgagatgctacgtggacgagggcatgcccaagacttgctttaatctcggcgggggtcgccgaggtgaaaacacctaacggtgttggggttacagcctgatagggtgctgcagaggcccacgaataggctagtataaaaatctctgctgtacatggcac

SEQ ID NO: 53 - DNA, 388 bp, Unknown
  Description of Unknown Bovine viral diarrhea virus 2 sequence
gtatacgagattagctaaagtactcgtatatggattggacgtcaacaaatttttaattggcaacgtagggaaccttcccctcagcgaaggccgaaaagaggctagccatgccctttagtaggactagcaaaagtagggggactagcggtagcagtgagttcgttggatggccgaacccctgagtacaggggagtcgtcaatggttcgacactccattagtcgaggagtctcgagatgccatgtggacgagggcatgcccacggcacatcttaacccatgcgggggttgcatgggtgaaagcgctaatcgtggcgttatggacacagcctgatagggtgtagcagagacctgctattccgctagtaaaaaactctgctgtacatggcac

SEQ ID NO: 54 - DNA, 373 bp, Unknown
  Description of Unknown Classical swine fever virus sequence
gtatacgaggttagttcattctcgtatgcattattggacaaatcaaaatttcaatttggttcagggcctccctccagcgacggccgaactgggctagccatgcccatagtaggactagcaaacggagggactagccgtagtggcgagctccctgggtgttctaagtcctgagtacaggacagtcgtcagtagttcgacgtgagcagaagcccacctcgagatgctatgtggacgagggcatgcccaagacgcaccttaaccctagcgggggtcgctagggtgaaatcacaccacgtgatgggagtccgacctgatagggtgctgcagaggctcactattaggctagtataaaaatctctgctgtacatggcac

SEQ ID NO: 55 - DNA, 191 bp, Unknown
  Description of Unknown Human blood-associated dicistrovirus sequence
aaaaccgaccccagagatcagaaagtcgttgacgcgatcttttattagaggacgttgcgctggcgcgagctttaattagcagacgccaaaaataaacaacaaaatgctgatcgcgagacttaattgtcagacgattggccaaatccgatgtgatctttgctgctcccagattgccgaaataggagtagtag

SEQ ID NO: 56 - DNA, 438 bp, Unknown
  Description of Unknown Hubei Picorna-like virus sequence
ccccaaaaccccccccttaaactcaacactgtagtggattcattttccgttgcaaaacaaaacattactacccgcatttatgtaggctctgtgttttctatgcgaccgttacattaatctctactctgacccactagtttataaaaccgaagacctgaatgaaacgattttccttcttttcaacctctaacgaacctctgacggcttgagaaacctgaagttagtaattatgtttaaaagaaaggaaagtcaaacgcgatgactcttacatccctattccataccgttgctccacaatgtgagcgatgcgaggtcgggactgcagtattaggggaacgagctacatggagagttaattatctctcccctcctacgggagtctcatgtgagctgtagaaagcggttggcacctctcgttacctcgcctgtacatgatcc

SEQ ID NO: 57 - DNA, 193 bp, Cricket paralysis virus
aaaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacct

SEQ ID NO: 58 - DNA, 752 bp, Unknown
  Description of Unknown Salivirus A sequence
tttcctcctttcgaccgccttacggcaggcgggtccgcggacggcttcggcctacccgcgacaagaatgccgtcatctgtccttatcacccatattctttcccttcccccgcaaccatcacgcttactcgcgcacgtgttgagtggcacgtgcgttgtccaaacagttacactcacacccttggggcgggtttgtcccgccctcgggttcctcgcggaaccctccctcttctctctccctttctatccgccttcactttccataactacagtgctttggtaggtaagcatcctgaccccccgcggaagctgccaacgtggcaactgtggggatccaggcaggttatcaaaggcacccggtctttccgccttcaggagtatccctgccggtgaattccgacagggctctgcttggtgccaacctcccccaaatgcgcgctgcgggagtgctcttccccaactcatcttagtaacctctcatgtgtgtgcttggtcagcatatctgaggcgacgttccgctgtcccagaccagtccagcaatggacgggccagtgtgcgtagtcgctttccggtttcccggcgcatgtttggcgaaacgctgaggtaaggttggtgtgcccaatgcccgtaatttggtgacacctcaagaccacccaggaatgccagggaggtaccccacttcggtgggatctgaccctgggctaattgtctacggtggttcttcttgcttccacttctcttttttctggcatg

SEQ ID NO: 59 - DNA, 708 bp, Unknown
  Description of Unknown Salivirus A sequence
tatggcaggcgggcttgtggacggcttcggcccacccacagcaagaatgccatcatctgtcctcacccccatgtttcccctttctttccctgcaaccgttacgcttactcgcaggtgcatttgagtggtgcacgtgttgaataaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttttccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttc

SEQ ID NO: 60 - DNA, 718 bp, Unknown
  Description of Unknown Salivirus A sequence
tttcgaccgccttatggcaggcgggcttgtggacggcttcggcccacccacagcaagaatgccatcatctgtcctcacccccatttctcccctccttcccctgcaaccattacgcttactcgcatgtgcattgagtggtgcacgtgttgaacaaacagctacactcacgtgggggcgggttttcccgcccttcggcctctcgcgaggcccacccttccccttcctcccataactacagtgctttggtaggtaagcatcctgatcccccgcggaagctgctcgcgtggcaactgtggggacccagacaggttatcaaaggcacccggtctttccgcctccaggagtatccctgctagtgaattctagtggggctctgcttggtgccaacctcccccaaatgcgcgctgcgggagtgctcttccccaactcaccctagtatcctctcatgtgtgtgcttggtcagcatatctgagacgatgttccgctgtcccagaccagtccagcaatggacgggccagtgtgcgtagtcgtcttccggcttgtccggcgcatgtttggtgaaccggtggggtaaggttggtgtgcccaacgcccgtactttggtgacaactcaagaccacccaggaatgccagggaggtaccccgcctcacggcgggatctgaccctgggctaattgtctacggtggttcttcttgcttccatttctttcttctgttc

SEQ ID NO: 61 - DNA, 699 bp, Unknown
  Description of Unknown Salivirus A sequence
tatggcaggcgggcttgtggacggtttcggcccacccacagcaagaatgccatcatctgtcctcacccccaattttccctttcttcccctgcaatcatcacgcttactcgcatgtgcattgagtggtgcatgtgttgaacaaacagctacactcacatgggggcgggttttcccgccctacggcctctcgcgaggcccacccttcccctccccttataactacagtgctttggcaggtaagcatcctgatcccccgcggaagctgctcacgtggcaactgtggggacccagacaggttatcaaaggcacccggtctttccgccttcaggagcatccccactagtgaattctagtggggctctgcttggtgccaacctcccccaaatgcgcgctgcgggagtgctcttccccaacccatcctagtatcctctcatgtgtgtgcttggtcagcatatctgagacgacgttccgctgtcccagaccagtccagtaatggacgggccagtgtgcgtagtcgtcttccggcttgtccggcgcatgtttggtgaaccggtggggtaaggttggtgtgcccaacgcccgtactttggtgacacctcaagaccacccaggaatgccagggaggtaccccgcctcacggcgggatctgaccctgggctaattgtctacggtggttcttcttgcttccacttctttctt

SEQ ID NO: 62 - DNA, 624 bp, Unknown
  Description of Unknown Salivirus A sequence
ttctcctgcaaccattacgcttaatcgcatgtgcattgagtggtgcatgtgttgaacaaacagctacaatcacatgggggcgggttttcccgccccacggcttctcgcgaggcccatccctcccttttctcccataactacagtgctttggtaggtaagcatcccgatctcccgcggaagctgctcacgtggcaactgtggggacccagacaggttatcaaaggcacccggtctttccgccttcaggagtatccctgctagcgaattctagtagggctctgcttggtgccaacctctcccaaatgcgcgctgcgggagtgctcttccccaaatcaccccagtatcctctcatgtgtgtgcctggtcagcatatctgagacgatgttccgctgtcccagaccagtccagtaatggacgggccagtgtgcgtagtcgtcctccggcttgtccggcgcatgtttggtgaaccggtggggtaaggttggtgtgcccaacgcccgtaatcaggggatacctcaaggcacccaggaatgccagggaggtatcccgcctcacagcgggatctgaccctggggtaaatgtctgcggggggtcctcttggcccaattctcagtaattttcagg

SEQ ID NO: 63 - DNA, 650 bp, Unknown
  Description of Unknown Salivirus A sequence
tctgtcctcaccccatcttcccttctttcctgcaccgttacgcttactcgcatgtgcattgagtggtgcacgtgcttgaacaaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttcatg

SEQ ID NO: 64 - DNA, 730 bp, Unknown; identical to SEQ ID NO: 17
  Description of Unknown Salivirus FHB sequence

SEQ ID NO: 65 - DNA, 741 bp, Unknown
  Description of Unknown CVB3 sequence
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 66 - DNA, 741 bp, Unknown
  Description of Unknown CVB1 sequence
ttaaaacagcctgtgggttgttcccacccacaggcccattgggcgctagcactctggtatcacggtacctttgtgcgcctgttttacatcccctccccaaattgtaatttagaagtttcacacaccgatcattagcaagcgtggcacaccagccatgttttgatcaagcacttctgttaccccggactgagtatcaatagaccgctaacgcggttgaaggagaaaacgttcgttacccggccaactacttcgaaaaacctagtaacaccatggaagttgcggagtgtttcgctcagcactaccccagtgtagatcaggtcgatgagtcaccgcgttccccacgggcgaccgtggcggtggctgcgttggcggcctgcctacggggaaacccgtaggacgctctaatacagacatggtgcgaagagtctattgagctagttggtaatcctccggcccctgaatgcggctaatcctaactgcggagcacataccctcaaaccagggggcagtgtgtcgtaacgggcaactctgcagcggaaccgactactttgggtgtccgtgtttcattttattcctatactggctgcttatggtgacaattgacaggttgttaccatatagttattggattggccatccggtgactaacagagcaattatatatctctttgttgggtttataccacttagcttgaaagaggttaaaacactacatctcatcattaaactaaatacaacaaa

SEQ ID NO: 67 - DNA, 742 bp, Unknown
  Description of Unknown Echovirus 7 sequence
ttaaaacagcctgtgggttgttcccacccacagggcccattgggcgtcagcaccctggtatcacggtacctttgtgcgcctgttttatatcccttcccccaattgtaacttagaagaaacacacaccgatcaacagcaagcgtggcacaccagccatgttttggtcaagcacttctgttaccccggactgagtatcaatagactgctcacgcggttgaaggagaaagcgtccgttatccggccagctacttcgagaaacctagtaacaccatggaagttgcggagtgtttcgctcagcactaccccagtgtagatcaggtcgatgagtcaccgctttccccacgggcgaccgtggcggtggctgcgttggcggcctgcctatgggggaacccataggacgctctaatacagacatggtgcgaagagtctattgagctagctggtattcctccggcccctgaatgcggctaatcctaactgtggagcacatgcccctaatccaaggggtagtgtgtcgtaatgagcaattccgcagcggaaccgactactttgggtgtccgtgtttcctcttattcttgtactggctgcttatggtgacaattgagagattgttaccatatagctattggattggccatccggtgactaatagagctattgtgtatctctttgttggatttgtaccacttaatttgaaagaaatcaggacactacgctacattttactattgaacaccgcaaa

SEQ ID NO: 68 - DNA, 743 bp, Unknown
  Description of Unknown CVB5 sequence
ttaaaacagcctgtgggttgtacccacccacagggcccactgggcgctagcactctggtatcacggtacctttgtgcgcctgttttatgcccccttcccccaattgaaacttagaagttacacacaccgatcaacagcgggcgtggcataccagccgcgtcttgatcaagcactcctgtttccccggaccgagtatcaatagactgctcacgcggttgaaggagaaaacgttcgttacccggctaactacttcgagaaacctagtagcatcatgaaagttgcgaagcgtttcgctcagcacatccccagtgtagatcaggtcgatgagtcaccgcattccccacgggcgaccgtggcggtggctgcgttggcggcctgcctacggggcaacccgtaggacgcttcaatacagacatggtgcgaagagtcgattgagctagttagtagtcctccggcccctgaatccggctaatcctaactgcggagcacataccctcaacccagggggcattgtgtcgtaacgggtaactctgcagcggaaccgactactttgggtgtccgtgtttccttttattcttataatggctgcttatggtgacaattgaaagattgttaccatatagctattggattggccatccggtgtctaacagagctattatatacctctttgttggatttgtaccacttgatctaaaggaagtcaagacactacaattcatcatacaattgaacacagcaaa

SEQ ID NO: 69 - DNA, 743 bp, Unknown
  Description of Unknown Enterovirus A71 sequence
ttaaaacagcctgtgggttgcacccactcacagggcccactgggcgcaagcactctggcacttcggtacctttgtgcgcctgttttatatcccctcccccaatgaaatttagaagcagcaaaccccgatcaatagcaggcataacgctccagttatgtcttgatcaagcacttctgtttccccggactgagtatcaatagactgctcacgcggttgaaggagaaaacgttcgttatccggctaactacttcggaaagcctagtaacaccatggaagttgcggagagtttcgttcagcacttccccagtgtagatcaggtcgatgagtcaccgcattccccacgggcgaccgtggcggtggctgcgttggcggcctgcccatggggtaacccatgggacgctctaatacggacatggtgtgaagagtctactgagctagttagtagtcctccggcccctgaatgcggctaatcccaactgcggagcacacgcccacaagccagtgggtagtgtgtcgtaacgggcaactctgcagcggaaccgactactttgggtgtccgtgtttccttttattcttatgttggctgcttatggtgacaattaaagagttgttaccatatagctattggattggccatccggtgtgcaacagagcgatcgtttacctatttattggttttgtaccattgacactgaagtctgtgatcacccttaattttatcttaaccctcaacacagccaaac

SEQ ID NO: 70 - DNA, 742 bp, Unknown
  Description of Unknown CVA3 sequence
ttaaaacagcctgtgggttgtacccacccacagggcccactgggcgctagcacactggtattacggtacctttgtgcgcctgttttataccccccccaacctcgaaacttagaagtaaagcaaacccgatcaatagcaggtgcggcgcaccagtcgcatcttgatcaagcacttctgtaaccccggaccgagtatcaatagactgctcacgcggttgaaggagaaaacgttcgttacccggctaactacttcgagaaacccagtagcatcatgaaagttgcagagtgtttcgctcagcactacccccgtgtagatcaggccgatgagtcaccgcacttccccacgggcgaccgtggcggtggctgcgttggcggcctgcctatggggcaacccataggacgctctaatacggacatggtgcgaagagtctattgagctagttagtagtcctccggcccctgaatgcggctaatcctaactgcggagcacatacccttaatccaaagggcagtgtgtcgtaacgggtaactctgcagcggaaccgactactttgggtgtccgtgtttccttttaatttttactggctgcttatggtgacaattgaggaattgttgccatatagctattggattggccatccggtgactaacagagctattgtgttccaatttgttggatttaccccgctcacactcacagtcgtaagaacccttcattacgtgttatttctcaactcaagaaa

SEQ ID NO: 71 - DNA, 745 bp, Unknown
  Description of Unknown CVA12 sequence
ttaaaacagcctgtgggttgtacccacccacagggcccactgggcgctagcactctggtactacggtacctttgtgtgcctgttttaagcccctaccccccactcgtaacttagaaggcttctcacactcgatcaatagtaggtgtggcacgccagtcacaccgtgatcaagcacttctgttaccccggtctgagtaccaataagctgctaacgcggctgaaggggaaaacgatcgttatccggctaactacttcgagaaacccagtaccaccatgaacgttgcagggtgtttcgctcggcacaaccccagtgtagatcaggtcgatgagtcaccgtattccccacgggcgaccgtggcggtggctgcgttggcggcctgcccatggggtgacccatgggacgctctaatactgacatggtgcgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttccttttattcttacattggctgcttatggtgacaattgaaaagttgttaccatatagctattggattggccatccggtgacaaatagagctattgtatatctttttgttggttacgtaccccttaattacaaagtggtttcaactttgaaatacatcctaacactaaattgtagaaa

SEQ ID NO: 72 - DNA, 742 bp, Unknown
  Description of Unknown Echovirus E24 sequence
ttaaaacagcctgtgggttgcacccacccacagggcccacagggcgctagcactctggtatcacggtacctttgtgcgcctgttttattaccccttccccaattgaaaattagaagcaatgcacaccgatcaacagcaggcgtggcgcaccagtcacgtctcgatcaagcacttctgtttccccggaccgagtatcaatagactgctcacgcggttgaaggagaaagtgttcgttatccggctaaccacttcgagaaacccagtaacaccatgaaagttgcagggtgtttcgctcagcacttccccagtgtagatcaggtcgatgagtcaccgcgttccccacgggcgaccgtggcggtggctgcgttggcggcctgcctatgggttaacccataggacgctctaatacagacatggtgcgaagagtttattgagctggttagtatccctccggcccctgaatgcggctaatcctaactgcggagcacgtgcctccaatccagggggttgcatgtcgtaacgggtaactctgcagcggaaccgactactttgggtgtccgtgtttccttttattcttatactggctgcttatggtgacaatcgaggaattgttaccatatagctattggattggccatccggtgtctaacagagcgattatatacctctttgttggatttatgcagctcaataccaccaactttaacacattgaaatatatcttaaagttaaacacagcaaa

SEQ ID NO: 73 - DNA, 240 bp, Anabaena sp.
gaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 74 - DNA, 239 bp, Anabaena sp.
aagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 75 - DNA, 238 bp, Anabaena sp.
agaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 76 - DNA, 190 bp, Anabaena sp.
gttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 77 - DNA, 189 bp, Anabaena sp.
ttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 78 - DNA, 188 bp, Anabaena sp.
tatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 79 - DNA, 187 bp, Anabaena sp.
atagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 80 - DNA, 186 bp, Anabaena sp.
tagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 81 - DNA, 139 bp, Anabaena sp.
acaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 82 - DNA, 138 bp, Anabaena sp.
caatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 83 - DNA, 137 bp, Anabaena sp.
aatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 84 - DNA, 136 bp, Anabaena sp.
atagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 85 - DNA, 135 bp, Anabaena sp.
tagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 86 - DNA, 134 bp, Anabaena sp.
agatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 87 - DNA, 133 bp, Anabaena sp.
gatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 88 - DNA, 132 bp, Anabaena sp.
atgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 89 - DNA, 131 bp, Anabaena sp.
tgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 90 - DNA, 78 bp, Anabaena sp.
caagacgagggtaaagagagagtccaattctcaaagccaataggcagtagcgaaagctgcaagagaatgaaaatccgt

SEQ ID NO: 91 - DNA, 77 bp, Anabaena sp.
aagacgagggtaaagagagagtccaattctcaaagccaataggcagtagcgaaagctgcaagagaatgaaaatccgt

SEQ ID NO: 92 - DNA, 76 bp, Anabaena sp.
agacgagggtaaagagagagtccaattctcaaagccaataggcagtagcgaaagctgcaagagaatgaaaatccgt

SEQ ID NO: 93 - DNA, 75 bp, Anabaena sp.
gacgagggtaaagagagagtccaattctcaaagccaataggcagtagcgaaagctgcaagagaatgaaaatccgt

SEQ ID NO: 94 - DNA, 74 bp, Anabaena sp.
acgagggtaaagagagagtccaattctcaaagccaataggcagtagcgaaagctgcaagagaatgaaaatccgt

SEQ ID NO: 95 - DNA, 40 bp, Anabaena sp.
aataggcagtagcgaaagctgcaagagaatgaaaatccgt

SEQ ID NO: 96 - DNA, 39 bp, Anabaena sp.
ataggcagtagcgaaagctgcaagagaatgaaaatccgt

SEQ ID NO: 97 - DNA, 38 bp, Anabaena sp.
taggcagtagcgaaagctgcaagagaatgaaaatccgt

SEQ ID NO: 98 - DNA, 37 bp, Anabaena sp.
aggcagtagcgaaagctgcaagagaatgaaaatccgt

SEQ ID NO: 99 - DNA, 36 bp, Anabaena sp.
ggcagtagcgaaagctgcaagagaatgaaaatccgt

SEQ ID NO: 100 - DNA, 27 bp, Anabaena sp.
gaaagctgcaagagaatgaaaatccgt

SEQ ID NO: 101 - DNA, 26 bp, Anabaena sp.
aaagctgcaagagaatgaaaatccgt

SEQ ID NO: 102 - DNA, 25 bp, Anabaena sp.
aagctgcaagagaatgaaaatccgt

SEQ ID NO: 103 - DNA, 24 bp, Anabaena sp.
agctgcaagagaatgaaaatccgt

SEQ ID NO: 104 - DNA, 23 bp, Anabaena sp.
gctgcaagagaatgaaaatccgt

SEQ ID NO: 105 - DNA, 22 bp, Anabaena sp.
ctgcaagagaatgaaaatccgt

SEQ ID NO: 106 - DNA, 18 bp, Anabaena sp.
aagagaatgaaaatccgt

SEQ ID NO: 107 - DNA, 17 bp, Anabaena sp.
agagaatgaaaatccgt

SEQ ID NO: 108 - DNA, 16 bp, Anabaena sp.
gagaatgaaaatccgt

SEQ ID NO: 109 - DNA, 35 bp, Anabaena sp.
gcagtagcgaaagctgcaagagaatgaaaatccgt

SEQ ID NO: 110 - DNA, 33 bp, Anabaena sp.
agtagcgaaagctgcaagagaatgaaaatccgt

SEQ ID NO: 111 - DNA, 32 bp, Anabaena sp.
gtagcgaaagctgcaagagaatgaaaatccgt

SEQ ID NO: 112 - DNA, 26 bp, Anabaena sp.
acggacttaaataattgagccttaaa

SEQ ID NO: 113 - DNA, 27 bp, Anabaena sp.
acggacttaaataattgagccttaaag

SEQ ID NO: 114 - DNA, 28 bp, Anabaena sp.
acggacttaaataattgagccttaaaga

SEQ ID NO: 115 - DNA, 76 bp, Anabaena sp.
acggacttaaataattgagccttaaagaagaaattctttaagtggatgctctcaaactcagggaaacctaaatcta

SEQ ID NO: 116 - DNA, 77 bp, Anabaena sp.
acggacttaaataattgagccttaaagaagaaattctttaagtggatgctctcaaactcagggaaacctaaatctag

SEQ ID NO: 117 - DNA, 78 bp, Anabaena sp.
acggacttaaataattgagccttaaagaagaaattctttaagtggatgctctcaaactcagggaaacctaaatctagt

SEQ ID NO: 118 - DNA, 79 bp, Anabaena sp.
acggacttaaataattgagccttaaagaagaaattctttaagtggatgctctcaaactcagggaaacctaaatctagtt

SEQ ID NO: 119 - DNA, 80 bp, Anabaena sp.
acggacttaaataattgagccttaaagaagaaattctttaagtggatgctctcaaactcagggaaacctaaatctagtta

SEQ ID NO: 120 - DNA, 127 bp, Anabaena sp.
acggacttaaataattgagccttaaagaagaaattctttaagtggatgctctcaaactcagggaaacctaaatctagttatagacaaggcaatcctgagccaagccgaagtagtaattagtaagtta

SEQ ID NO: 121 - DNA, 128 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaa

SEQ ID NO: 122 - DNA, 129 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaac

SEQ ID NO: 123 - DNA, 130 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaaca

SEQ ID NO: 124 - DNA, 131 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaa

SEQ ID NO: 125 - DNA, 132 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaat

SEQ ID NO: 126 - DNA, 133 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaata

SEQ ID NO: 127 - DNA, 134 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatag

SEQ ID NO: 128 - DNA, 135 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaataga

SEQ ID NO: 129 - DNA, 188 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgt

SEQ ID NO: 130 - DNA, 189 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtc

SEQ ID NO: 131 - DNA, 190 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtca

SEQ ID NO: 132 - DNA, 191 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaa

SEQ ID NO: 133 - DNA, 192 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaag

SEQ ID NO: 134 - DNA, 226 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagcc

SEQ ID NO: 135 - DNA, 227 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagcca

SEQ ID NO: 136 - DNA, 228 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaa

SEQ ID NO: 137 - DNA, 229 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaat

SEQ ID NO: 138 - DNA, 230 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaata

SEQ ID NO: 139 - DNA, 239 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagc

SEQ ID NO: 140 - DNA, 240 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcg

SEQ ID NO: 141 - DNA, 241 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcga

SEQ ID NO: 142 - DNA, 242 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaa

SEQ ID NO: 143 - DNA, 243 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaa

SEQ ID NO: 144 - DNA, 244 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaag

SEQ ID NO: 145 - DNA, 248 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgc

SEQ ID NO: 146 - DNA, 249 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgca

SEQ ID NO: 147 - DNA, 250 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaa

SEQ ID NO: 148 - DNA, 231 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatag

SEQ ID NO: 149 - DNA, 233 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggc

SEQ ID NO: 150 - DNA, 234 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggca

SEQ ID NO: 151 - DNA, 104 bp, Azoarcus sp.
tgcgccgatgaaggtgtagagactagacggcacccacctaaggcaaacgctatggtgaaggcatagtccagggagtggcgaaagtcacacaaaccggaatccgt

SEQ ID NO: 152 - DNA, 141 bp, Azoarcus sp.
ccgggcgtatggcaacgccgagccaagcttcggcgcctgcgccgatgaaggtgtagagactagacggcacccacctaaggcaaacgctatggtgaaggcatagtccagggagtggcgaaagtcacacaaaccggaatccgt

SEQ ID NO: 153 - DNA, 78 bp, Azoarcus sp.
acggcacccacctaaggcaaacgctatggtgaaggcatagtccagggagtggcgaaagtcacacaaaccggaatccgt

SEQ ID NO: 154 - DNA, 58 bp, Azoarcus sp.
acgctatggtgaaggcatagtccagggagtggcgaaagtcacacaaaccggaatccgt

SEQ ID NO: 155 - DNA, 75 bp, Thiomargarita sp.
attaaagttatagaattatcagagaatgatatagtccaagccttatggtaacatgagggcacttgaccctggtag

SEQ ID NO: 156 - DNA, 195 bp, Staphylococcus virus Twort
aagatgtaggcaatcctgagctaagctcttagtaataagagaaagtgcaacgactattccgataggaagtagggtcaagtgactcgaaatggggattacccttctagggtagtgatatagtctgaacatatatggaaacatatagaaggataggagtaacgaacctattcgtaacataattgaacttttagttat

SEQ ID NO: 157 - DNA, 163 bp, Staphylococcus virus Twort
taataagagaaagtgcaacgactattccgataggaagtagggtcaagtgactcgaaatggggattacccttctagggtagtgatatagtctgaacatatatggaaacatatagaaggataggagtaacgaacctattcgtaacataattgaacttttagttat

SEQ ID NO: 158 - DNA, 133 bp, Staphylococcus virus Twort
taggaagtagggtcaagtgactcgaaatggggattacccttctagggtagtgatatagtctgaacatatatggaaacatatagaaggataggagtaacgaacctattcgtaacataattgaacttttagttat

SEQ ID NO: 159 - DNA, 92 bp, Staphylococcus virus Twort
ctagggtagtgatatagtctgaacatatatggaaacatatagaaggataggagtaacgaacctattcgtaacataattgaacttttagttat

SEQ ID NO: 160 - DNA, 92 bp, Unknown
  Description of Unknown LSUp1 sequence
agttaataaagatgatgaaatagtctgaaccattttgagaaaagtggaaataaaagaaaatcttttatgataacataaattgaacaggctaa

SEQ ID NO: 161 - DNA, 62 bp, Unknown
  Description of Unknown Bacteriophage Phi I sequence
caaagactgatgatatagtccgacactcctagtaataggagaatacagaaaggatgaaatcc

SEQ ID NO: 162 - DNA, 76 bp, Nostoc sp.
agtcgagggtaaagggagagtccaattctcaaagcctattggcagtagcgaaagctgcgggagaatgaaaatccgt

SEQ ID NO: 163 - DNA, 76 bp, Nostoc sp.
agccgagggtaaagggagagtccaattctcaaagccaataggcagtagcgaaagctgcgggagaatgaaaatccgt

SEQ ID NO: 164 - DNA, 89 bp, Nodularia sp.
agccgagggtaaagggagagtccaattctcaaagccgaaggttattaaaacctggcagcagtgaaagctgcgggagaatgaaaatccgt

SEQ ID NO: 165 - DNA, 79 bp, Pleurocapsa sp.
agctgagggtaaagagagagtccaattctcaaagccagcagatggcagtagcgaaagctgcgggagaatgaaaatccgt

SEQ ID NO: 166 - DNA, 76 bp, Planktothrix sp.
agccgagggtaaagagagagtccaattctcaaagccaattggtagtagcgaaagctacgggagaatgaaaatccgt

SEQ ID NO: 167 - DNA, 118 bp, Azoarcus sp.
gcggactcatatttcgatgtgccttgcgccgggaaaccacgcaagggatggtgtcaaattcggcgaaacctaagcgcccgcccgggcgtatggcaacgccgagccaagcttcggcgcc

SEQ ID NO: 168 - DNA, 81 bp, Azoarcus sp.
gcggactcatatttcgatgtgccttgcgccgggaaaccacgcaagggatggtgtcaaattcggcgaaacctaagcgcccgc

SEQ ID NO: 169 - DNA, 144 bp, Azoarcus sp.
gcggactcatatttcgatgtgccttgcgccgggaaaccacgcaagggatggtgtcaaattcggcgaaacctaagcgcccgcccgggcgtatggcaacgccgagccaagcttcggcgcctgcgccgatgaaggtgtagagactag

SEQ ID NO: 170 - DNA, 164 bp, Azoarcus sp.
gcggactcatatttcgatgtgccttgcgccgggaaaccacgcaagggatggtgtcaaattcggcgaaacctaagcgcccgcccgggcgtatggcaacgccgagccaagcttcggcgcctgcgccgatgaaggtgtagagactagacggcacccacctaaggcaa

SEQ ID NO: 171 - DNA, 179 bp, Thiomargarita sp.
aggattagatactacactaagtgtcccccagactggtgacagtctggtgtgcatccagctatatcggtgaaaccccattggggtaataccgagggaagctatattatatatatattaataaatagccccgtagagactatgtaggtaaggagatagaagatgataaaatcaaaatcatc

SEQ ID NO: 172 - DNA, 84 bp, Staphylococcus virus Twort
actactgaaagcataaataattgtgcctttatacagtaatgtatatcgaaaaatcctctaattcagggaacacctaaacaaact

SEQ ID NO: 173 - DNA, 116 bp, Staphylococcus virus Twort
actactgaaagcataaataattgtgcctttatacagtaatgtatatcgaaaaatcctctaattcagggaacacctaaacaaactaagatgtaggcaatcctgagctaagctcttag

SEQ ID NO: 174 - DNA, 146 bp, Staphylococcus virus Twort
actactgaaagcataaataattgtgcctttatacagtaatgtatatcgaaaaatcctctaattcagggaacacctaaacaaactaagatgtaggcaatcctgagctaagctcttagtaataagagaaagtgcaacgactattccga

SEQ ID NO: 175 - DNA, 187 bp, Staphylococcus virus Twort
actactgaaagcataaataattgtgcctttatacagtaatgtatatcgaaaaatcctctaattcagggaacacctaaacaaactaagatgtaggcaatcctgagctaagctcttagtaataagagaaagtgcaacgactattccgataggaagtagggtcaagtgactcgaaatggggattaccctt

SEQ ID NO: 176 - DNA, 216 bp, Unknown
  Description of Unknown LSUp1 sequence
cgctagggatttataactgtgagtcctccaatattataaaatgttggtaatatattgggtaaatttcaaagacaacttttctccacgtcaggatatagtgtatttgaagcgaaacttattttagcagtgaaaaagcaaataaggacgttcaacgactaaaaggtgagtattgctaacaataatccttttttttaatgcccaacatctttattaact

SEQ ID NO: 177 - DNA, 559 bp, Unknown
  Description of Unknown Bacteriophage Phi I sequence
gtgggtgcataaactatttcattgtgcacattaaatctggtgaactcggtgaaaccctaatggggcaataccgagccaagccatagggaggatatatgagaggcaagaagttaattcttgaggccactgagactggctgtatcatccctacgtcacacaaacttaatgccgatggttatttcagaaagaaaaccaatggcgtcttagagatgtatcacagaacggtgtggaaggagcataacggagacatacctgatggcttcgagatagaccataagtgtcgcaatagggcttgctgtaatatagagcatttacagatgcttgagggtacagcccacactgttaagaccaatcgtgaacgctacgcagacagaaaggaaacagctagggaatactggctggagactggatgtaccggcctagcactcggtgagaagtttggtgtgtcgttctcttctgcttgtaagtggattagagaatggaaggcgtagagactatccgaaaggagtagggccgagggtgagactccctcgtaacccgaagcgccagacagtcaact

SEQ ID NO: 178 - DNA, 218 bp, Nostoc sp.
acggacttaagtaattgagccttaaagaagaaattctttaagtggcagctctcaaactcagggaaacctaaatctgttcacagacaaggcaatcctgagccaagccgaaagagtcatgagtgctgagtagtgagtaaaataaaagctcacaactcagaggttgtaactctaagctagtcggaaggtgcagagactcgacgggagctaccctaacgtaa

SEQ ID NO: 179 - DNA, 165 bp, Nostoc sp.
acggacttaaactgaattgagccttagagaagaaattctttaagtgtcagctctcaaactcagggaaacctaaatctgttgacagacaaggcaatcctgagccaagccgagaactctaagttattcggaaggtgcagagactcgacgggagctaccctaacgtca

SEQ ID NO: 180 - DNA, 181 bp, Nodularia sp.
acggacttagaaaactgagccttgatcgagaaatctttcaagtggaagctctcaaattcagggaaacctaaatctgtttacagatatggcaatcctgagccaagccgaaacaagtcctgagtgttaaagctcataactcatcggaaggtgcagagactcgacgggagctaccctaacgtta

SEQ ID NO: 181 - DNA, 188 bp, Pleurocapsa sp.
acggacttaaaaaaattgagccttggcagagaaatctgtcatgcgaacgctctcaaattcagggaaacctaagtctggcaacagatatggcaatcctgagccaagccttaatcaaggaaaaaaacatttttaccttttaccttgaaaggaaggtgcagagactcaacgggagctaccctaacaggtca

SEQ ID NO: 182 - DNA, 200 bp, Planktothrix sp.
acggacttaaagataaattgagccttgaggcgagaaatctctcaagtgtaagctgtcaaattcagggaaacctaaatctgtaaattcagacaaggcaatcctgagccaagcctaggggtattagaaatgagggagtttccccaatctaagatcaatacctaggaaggtgcagagactcgacgggagctaccctaacgtta

SEQ ID NO: 183 - DNA, 166 bp, Artificial Sequence
  Description of Artificial Sequence Synthetic polynucleotide
agtatataagaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 184 - DNA, 176 bp, Artificial Sequence
  Description of Artificial Sequence Synthetic polynucleotide
ctgaaattatacttatactcaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 185 - DNA, 186 bp, Artificial Sequence
  Description of Artificial Sequence Synthetic polynucleotide
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 186 - DNA, 196 bp, Artificial Sequence
  Description of Artificial Sequence Synthetic polynucleotide
catcaacaatatgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 187 - DNA, 206 bp, Artificial Sequence
  Description of Artificial Sequence Synthetic polynucleotide
catcaacaatatgaaactatacttatactcagtatatgaagcattatcgcaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 188 - DNA, 166 bp, Artificial Sequence
  Description of Artificial Sequence Synthetic polynucleotide
tagcgtcagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 189 - DNA, 176 bp, Artificial Sequence
  Description of Artificial Sequence Synthetic polynucleotide
atactcatactagcgtcagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 190 - DNA, 186 bp, Artificial Sequence
  Description of Artificial Sequence Synthetic polynucleotide
gtgtgaagctatactcatactagcgtcagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 191 - DNA, 196 bp, Artificial Sequence
  Description of Artificial Sequence Synthetic polynucleotide
cctcacctgagtgtgaagctatactcatactagcgtcagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 192 - DNA, 206 bp, Artificial Sequence
  Description of Artificial Sequence Synthetic polynucleotide
ccgaatgatgcctcacctgagtgtgaagctatactcatactagcgtcagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 193 - DNA, 166 bp, Artificial Sequence
  Description of Artificial Sequence Synthetic polynucleotide
cggtgcgagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 194 - DNA, 176 bp, Artificial Sequence
  Description of Artificial Sequence Synthetic polynucleotide
cgctccgacccagtgcgagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 195 - DNA, 186 bp, Artificial Sequence
  Description of Artificial Sequence Synthetic polynucleotide
ctgaaattatactaatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 196 - DNA, 186 bp, Artificial Sequence
  Description of Artificial Sequence Synthetic polynucleotide
ctgaaaatatactaatactcactatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 197 - DNA, 186 bp, Artificial Sequence
  Description of Artificial Sequence Synthetic polynucleotide
ctgataatatagtaatactcactatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 198 - DNA, 186 bp, Artificial Sequence
  Description of Artificial Sequence Synthetic polynucleotide
ctgataataaagtaatacacactataagacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 199 - DNA, 186 bp, Artificial Sequence
  Description of Artificial Sequence Synthetic polynucleotide
ctgaaattatacttatactctctaagttacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 200 - DNA, 186 bp, Artificial Sequence
  Description of Artificial Sequence Synthetic polynucleotide
ctgaaattatgtgtgttacatctaagttacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 201 - DNA, 186 bp, Artificial Sequence
  Description of Artificial Sequence Synthetic polynucleotide
gttgatcggtgtgtgttacatctaagttacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 202 - DNA, 186 bp, Artificial Sequence
  Description of Artificial Sequence Synthetic polynucleotide
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtgattaaacag

SEQ ID NO: 203 - DNA, 196 bp, Artificial Sequence
  Description of Artificial Sequence Synthetic polynucleotide
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtgattcacaatataaattacg

SEQ ID NO: 204 - DNA, 186 bp, Artificial Sequence
  Description of Artificial Sequence Synthetic polynucleotide
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggatcatagc

SEQ ID NO: 205 - DNA, 196 bp, Artificial Sequence
  Description of Artificial Sequence Synthetic polynucleotide
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggatcgcagcataatatccg

SEQ ID NO: 206 - DNA, 196 bp, Artificial Sequence
  Description of Artificial Sequence Synthetic polynucleotide
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagcgcgcctaccg

SEQ ID NO: 207 - DNA, 219 bp, Artificial Sequence
  Description of Artificial Sequence Synthetic polynucleotide
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagcgcgcctaccgaaagccggcgtcgacgttagcgc

SEQ ID NO: 208 - DNA, 219 bp, Artificial Sequence
  Description of Artificial Sequence Synthetic polynucleotide
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggatcgcagcataatatccgaaacgaggatacaagtgacatgc

SEQ ID NO: 209 - DNA, 219 bp, Artificial Sequence
  Description of Artificial Sequence Synthetic polynucleotide
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtgattcacaatctaaattacgaaacgataaatgataactctaac

SEQ ID NO: 210 - DNA, 156 bp, Artificial Sequence
  Description of Artificial Sequence Synthetic polynucleotide
aaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 211 - DNA, 161 bp, Artificial Sequence
  Description of Artificial Sequence Synthetic polynucleotide
cgggcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 212 - DNA, 186 bp, Artificial Sequence
  Description of Artificial Sequence Synthetic polynucleotide
cgctccgacgagcttccggccagtgcgagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 213 - DNA, 57 bp, Artificial Sequence
  Description of Artificial Sequence Synthetic oligonucleotide
aaacaaaccacggcagtagcgaaagctgcaagagaatgaaaatccgtggctcgcagc

SEQ ID NO: 214 - DNA, 67 bp, Artificial Sequence
  Description of Artificial Sequence Synthetic oligonucleotide
agtatataagaaacaaaccacggcagtagcgaaagctgcaagagaatgaaaatccgtggctcgcagc

SEQ ID NO: 215 - DNA, 77 bp, Artificial Sequence
  Description of Artificial Sequence Synthetic oligonucleotide
ctgaaattatacttatactcaaacaaaccacggcagtagcgaaagctgcaagagaatgaaaatccgtggctcgcagc

SEQ ID NO: 216 - DNA, 87 bp, Artificial Sequence
  Description of Artificial Sequence Synthetic oligonucleotide
ctgaaattatacttatactcagtatatgacaaacaaaccacggcagtagcgaaagctgcaagagaatgaaaatccgtggctcgcagc

SEQ ID NO: 217 - DNA, 67 bp, Artificial Sequence
  Description of Artificial Sequence Synthetic oligonucleotide
tagcgtcagcaaacaaacaaaggcagtagcgaaagctgcaagagaatgaaaatccgtggctcgcagc

SEQ ID NO: 218 - DNA, 77 bp, Artificial Sequence
  Description of Artificial Sequence Synthetic oligonucleotide
atactcatactagcgtcagcaaacaaacaaaggcagtagcgaaagctgcaagagaatgaaaatccgtggctcgcagc

SEQ ID NO: 219 - DNA, 87 bp, Artificial Sequence
  Description of Artificial Sequence Synthetic oligonucleotide
gtgtgaagctatactcatactagcgtcagcaaacaaacaaaggcagtagcgaaagctgcaagagaatgaaaatccgtggctcgcagc

SEQ ID NO: 220 - DNA, 67 bp, Artificial Sequence
  Description of Artificial Sequence Synthetic oligonucleotide
cggtgcgagcaaacaaacaaaggcagtagcgaaagctgcaagagaatgaaaatccgtggctcgcagc

SEQ ID NO: 221 - DNA, 77 bp, Artificial Sequence
  Description of Artificial Sequence Synthetic oligonucleotide
cgctccgacccagtgcgagcaaacaaacaaaggcagtagcgaaagctgcaagagaatgaaaatccgtggctcgcagc

SEQ ID NO: 222 - DNA, 87 bp, Artificial Sequence
  Description of Artificial Sequence Synthetic oligonucleotide
cgctccgacgagcttccggccagtgcgagcaaacaaacaaaggcagtagcgaaagctgcaagagaatgaaaatccgtggctcgcagc

SEQ ID NO: 223 - DNA, 98 bp, Artificial Sequence
  Description of Artificial Sequence Synthetic oligonucleotide
aaacaaaccacaagacgagggtaaagagagagtccaattctcaaagccaataggcagtagcgaaagctgcaagagaatgaaaatccgtggctcgcagc

SEQ ID NO: 224 - DNA, 108 bp, Artificial Sequence
  Description of Artificial Sequence Synthetic polynucleotide
agtatataagaaacaaaccacaagacgagggtaaagagagagtccaattctcaaagccaataggcagtagcgaaagctgcaagagaatgaaaatccgtggctcgcagc

SEQ ID NO: 225 - DNA, 118 bp, Artificial Sequence
  Description of Artificial Sequence Synthetic polynucleotide
ctgaaattatacttatactcaaacaaaccacaagacgagggtaaagagagagtccaattctcaaagccaataggcagtagcgaaagctgcaagagaatgaaaatccgtggctcgcagc

SEQ ID NO: 226 - DNA, 128 bp, Artificial Sequence
  Description of Artificial Sequence Synthetic polynucleotide
ctgaaattatacttatactcagtatatgacaaacaaaccacaagacgagggtaaagagagagtccaattctcaaagccaataggcagtagcgaaagctgcaagagaatgaaaatccgtggctcgcagc

SEQ ID NO: 227 - DNA, 108 bp, Artificial Sequence
  Description of Artificial Sequence Synthetic polynucleotide
tagcgtcagcaaacaaacaaaaagacgagggtaaagagagagtccaattctcaaagccaataggcagtagcgaaagctgcaagagaatgaaaatccgtggctcgcagc

SEQ ID NO: 228 - DNA, 118 bp, Artificial Sequence
  Description of Artificial Sequence Synthetic polynucleotide
atactcatactagcgtcagcaaacaaacaaaaagacgagggtaaagagagagtccaattctcaaagccaataggcagtagcgaaagctgcaagagaatgaaaatccgtggctcgcagc

SEQ ID NO: 229 - DNA, 128 bp, Artificial Sequence
  Description of Artificial Sequence Synthetic polynucleotide
gtgtgaagctatactcatactagcgtcagcaaacaaacaaaaagacgagggtaaagagagagtccaattctcaaagccaataggcagtagcgaaagctgcaagagaatgaaaatccgtggctcgcagc

SEQ ID NO: 230 - DNA, 108 bp, Artificial Sequence
  Description of Artificial Sequence Synthetic polynucleotide
cggtgcgagcaaacaaacaaaaagacgagggtaaagagagagtccaattctcaaagccaataggcagtagcgaaagctgcaagagaatgaaaatccgtggctcgcagc

SEQ ID NO: 231 - DNA, 118 bp, Artificial Sequence
  Description of Artificial Sequence Synthetic polynucleotide
cgctccgacccagtgcgagcaaacaaacaaaaagacgagggtaaagagagagtccaattctcaaagccaataggcagtagcgaaagctgcaagagaatgaaaatccgtggctcgcagc

SEQ ID NO: 232 - DNA, 128 bp, Artificial Sequence
  Description of Artificial Sequence Synthetic polynucleotide
cgctccgacgagcttccggccagtgcgagcaaacaaacaaaaagacgagggtaaagagagagtccaattctcaaagccaataggcagtagcgaaagctgcaagagaatgaaaatccgtggctcgcagc

SEQ ID NO: 233 - DNA, 176 bp, Artificial Sequence
  Description of Artificial Sequence Synthetic polynucleotide
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 234 - DNA, 77 bp, Artificial Sequence
  Description of Artificial Sequence Synthetic oligonucleotide
ctgaaattatacttatactcagtatatgacaaacaaaccacggcagtagcgaaagctgcaagagaatgaaaatccgt

SEQ ID NO: 235 - DNA, 87 bp, Artificial Sequence
  Description of Artificial Sequence Synthetic oligonucleotide
ctgaaattatacttatactcagtatatgacaaacaaaccacggcagtagcgaaagctgcaagagaatgaaaatccgtgattaaacag

SEQ ID NO: 236 - DNA, 97 bp, Artificial Sequence
  Description of Artificial Sequence Synthetic oligonucleotide
ctgaaattatacttatactcagtatatgacaaacaaaccacggcagtagcgaaagctgcaagagaatgaaaatccgtgattcacaatataaattacg

SEQ ID NO: 237 - DNA, 87 bp, Artificial Sequence
  Description of Artificial Sequence Synthetic oligonucleotide
ctgaaattatacttatactcagtatatgacaaacaaaccacggcagtagcgaaagctgcaagagaatgaaaatccgtggatcatagc

SEQ ID NO: 238 - DNA, 97 bp, Artificial Sequence
  Description of Artificial Sequence Synthetic oligonucleotide
ctgaaattatacttatactcagtatatgacaaacaaaccacggcagtagcgaaagctgcaagagaatgaaaatccgtggatcgcagcataatatccg

SEQ ID NO: 239 - DNA, 97 bp, Artificial Sequence
  Description of Artificial Sequence Synthetic oligonucleotide
ctgaaattatacttatactcagtatatgacaaacaaaccacggcagtagcgaaagctgcaagagaatgaaaatccgtggctcgcagcgcgcctaccg

SEQ ID NO: 240 - DNA, 118 bp, Artificial Sequence
  Description of Artificial Sequence Synthetic polynucleotide
ctgaaattatacttatactcagtatatgacaaacaaaccacaagacgagggtaaagagagagtccaattctcaaagccaataggcagtagcgaaagctgcaagagaatgaaaatccgt

SEQ ID NO: 241 - DNA, 128 bp, Artificial Sequence
  Description of Artificial Sequence Synthetic polynucleotide
ctgaaattatacttatactcagtatatgacaaacaaaccacaagacgagggtaaagagagagtccaattctcaaagccaataggcagtagcgaaagctgcaagagaatgaaaatccgtgattaaacag

SEQ ID NO: 242 - DNA, 138 bp, Artificial Sequence
  Description of Artificial Sequence Synthetic polynucleotide
ctgaaattatacttatactcagtatatgacaaacaaaccacaagacgagggtaaagagagagtccaattctcaaagccaataggcagtagcgaaagctgcaagagaatgaaaatccgtgattcacaatataaattacg

SEQ ID NO: 243 - DNA, 128 bp, Artificial Sequence
  Description of Artificial Sequence Synthetic polynucleotide
ctgaaattatacttatactcagtatatgacaaacaaaccacaagacgagggtaaagagagagtccaattctcaaagccaataggcagtagcgaaagctgcaagagaatgaaaatccgtggatcatagc

SEQ ID NO: 244 - DNA, 138 bp, Artificial Sequence
  Description of Artificial Sequence Synthetic polynucleotide
ctgaaattatacttatactcagtatatgacaaacaaaccacaagacgagggtaaagagagagtccaattctcaaagccaataggcagtagcgaaagctgcaagagaatgaaaatccgtggatcgcagcataatatccg

SEQ ID NO: 245 - DNA, 138 bp, Artificial Sequence
  Description of Artificial Sequence Synthetic polynucleotide
ctgaaattatacttatactcagtatatgacaaacaaaccacaagacgagggtaaagagagagtccaattctcaaagccaataggcagtagcgaaagctgcaagagaatgaaaatccgtggctcgcagcgcgcctaccg

SEQ ID NO: 246 - DNA, 162 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaacttatatact

SEQ ID NO: 247 - DNA, 172 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagagtataagtataatttcag

SEQ ID NO: 248 - DNA, 182 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 249 - DNA, 192 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcatattgttgatg

SEQ ID NO: 250 - DNA, 202 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagcgataatgcttcatatactgagtataagtatagtttcatattgttgatg

SEQ ID NO: 251 - DNA, 162 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctgacgcta

SEQ ID NO: 252 - DNA, 172 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctgacgctagtatgagtat

SEQ ID NO: 253 - DNA, 182 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctgacgctagtatgagtatagcttcacac

SEQ ID NO: 254 - DNA, 192 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctgacgctagtatgagtatagcttcacactcaggtgagg

SEQ ID NO: 255 - DNA, 202 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctgacgctagtatgagtatagcttcacactcaggtgaggcatcattcgg

SEQ ID NO: 256 - DNA, 162 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctcgcaccg

SEQ ID NO: 257 - DNA, 172 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctcgcactgggtcggagcg

SEQ ID NO: 258 - DNA, 182 bp, Artificial Sequence; identical to SEQ ID NO: 248
  Description of Artificial Sequence Synthetic polynucleotide

SEQ ID NO: 259 - DNA, 182 bp, Artificial Sequence; identical to SEQ ID NO: 248
  Description of Artificial Sequence Synthetic polynucleotide

SEQ ID NO: 260 - DNA, 182 bp, Artificial Sequence; identical to SEQ ID NO: 248
  Description of Artificial Sequence Synthetic polynucleotide

SEQ ID NO: 261 - DNA, 182 bp, Artificial Sequence; identical to SEQ ID NO: 248
  Description of Artificial Sequence Synthetic polynucleotide

SEQ ID NO: 262 - DNA, 182 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtaacttagagagtataagtataatttcag

SEQ ID NO: 263 - DNA, 182 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtaacttagatgtaacacacataatttcag

SEQ ID NO: 264 - DNA, 182 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtaacttagatgtaacacacaccgatcaac

SEQ ID NO: 265 - DNA, 182 bp, Artificial Sequence
  Description of Artificial Sequence Synthetic polynucleotide
ctgtttaatcacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 266 - DNA, 192 bp, Artificial Sequence
  Description of Artificial Sequence Synthetic polynucleotide
cgtaatttatattgtgaatcacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 267 - DNA, 182 bp, Artificial Sequence
  Description of Artificial Sequence Synthetic polynucleotide
gctatgatccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 268 - DNA, 192 bp, Artificial Sequence
  Description of Artificial Sequence Synthetic polynucleotide
cggatattatgctgcgatccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 269 - DNA, 192 bp, Artificial Sequence
  Description of Artificial Sequence Synthetic polynucleotide
cggtaggcgcgctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 270 - DNA, 215 bp, Artificial Sequence
  Description of Artificial Sequence Synthetic polynucleotide
gcgctaacgtcgacgccggcaaacggtaggcgcgctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 271 - DNA, 215 bp, Artificial Sequence
  Description of Artificial Sequence Synthetic polynucleotide
gcatgtcacttgtatcctcgaaacggatattatgctgcgatccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 272 - DNA, 215 bp, Artificial Sequence
  Description of Artificial Sequence Synthetic polynucleotide
gttagagttatcatttatcgaaacgtaatttagattgtgaatcacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 273 - DNA, 152 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaa

SEQ ID NO: 274 - DNA, 157 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagcccg

SEQ ID NO: 275 - DNA, 182 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctcgcactggccggaagctcgtcggagcg

SEQ ID NO: 276 - DNA, 251 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaa

SEQ ID NO: 277 - DNA, 261 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaacttatatact

SEQ ID NO: 278 - DNA, 271 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagagtataagtataatttcag

SEQ ID NO: 279 - DNA, 281 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagtcatatactgagtataagtataatttcag

SEQ ID NO: 280 - DNA, 261 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataaacaaaaacaagctgacgcta

SEQ ID NO: 281 - DNA, 271 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataaacaaaaacaagctgacgctagtatgagtat

SEQ ID NO: 282 - DNA, 281 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataaacaaaaacaagctgacgctagtatgagtatagcttcacac

SEQ ID NO: 283 - DNA, 261 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataaacaaaaacaagctcgcaccg

SEQ ID NO: 284 - DNA, 271 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataaacaaaaacaagctcgcactgggtcggagcg

SEQ ID NO: 285 - DNA, 281 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataaacaaaaacaagctcgcactggccggaagctcgtcggagcg

SEQ ID NO: 286 - DNA, 210 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaa

SEQ ID NO: 287 - DNA, 220 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaacttatatact

SEQ ID NO: 288 - DNA, 230 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagagtataagtataatttcag

SEQ ID NO: 289 - DNA, 240 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagtcatatactgagtataagtataatttcag

SEQ ID NO: 290 - DNA, 220 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaacaaaaacaagctgacgcta

SEQ ID NO: 291 - DNA, 230 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaacaaaaacaagctgacgctagtatgagtat

SEQ ID NO: 292 - DNA, 240 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaacaaaaacaagctgacgctagtatgagtatagcttcacac

SEQ ID NO: 293 - DNA, 220 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaacaaaaacaagctcgcaccg

SEQ ID NO: 294 - DNA, 230 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaacaaaaacaagctcgcactgggtcggagcg

SEQ ID NO: 295 - DNA, 240 bp, Artificial Sequence
  Description of Artificial Sequence Synthetic polynucleotide
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaacaaaaacaagctcgcactggccggaagctcgtcggagcg

SEQ ID NO: 296 - DNA, 172 bp, Artificial Sequence
  Description of Artificial Sequence Synthetic polynucleotide
acggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 297 - DNA, 271 bp, Artificial Sequence
  Description of Artificial Sequence Synthetic polynucleotide
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagtcatatactgagtataagtataatttcag

SEQ ID NO: 298 - DNA, 281 bp, Artificial Sequence
  Description of Artificial Sequence Synthetic polynucleotide
ctgtttaatcacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagtcatatactgagtataagtataatttcag

SEQ ID NO: 299 - DNA, 291 bp, Artificial Sequence
  Description of Artificial Sequence Synthetic polynucleotide
cgtaatttatattgtgaatcacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagtcatatactgagtataagtataatttcag

SEQ ID NO: 300 - DNA, 281 bp, Artificial Sequence
  Description of Artificial Sequence Synthetic polynucleotide
gctatgatccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagtcatatactgagtataagtataatttcag

SEQ ID NO: 301 - DNA, 291 bp, Artificial Sequence
  Description of Artificial Sequence Synthetic polynucleotide
cggatattatgctgcgatccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagtcatatactgagtataagtataatttcag

SEQ ID NO: 302 - DNA, 291 bp, Artificial Sequence
  Description of Artificial Sequence Synthetic polynucleotide
cggtaggcgcgctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagtcatatactgagtataagtataatttcag

SEQ ID NO: 303 - DNA, 230 bp, Artificial Sequence
  Description of Artificial Sequence Synthetic polynucleotide
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagtcatatactgagtataagtataatttcag

SEQ ID NO: 304 - DNA, 240 bp, Artificial Sequence
  Description of Artificial Sequence Synthetic polynucleotide
ctgtttaatcacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagtcatatactgagtataagtataatttcag

SEQ ID NO: 305 - DNA, 250 bp, Artificial Sequence
  Description of Artificial Sequence Synthetic polynucleotide
cgtaatttatattgtgaatcacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagtcatatactgagtataagtataatttcag

SEQ ID NO: 306 - DNA, 240 bp, Artificial Sequence
  Description of Artificial Sequence Synthetic polynucleotide
gctatgatccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagtcatatactgagtataagtataatttcag

SEQ ID NO: 307 - DNA, 250 bp, Artificial Sequence
  Description of Artificial Sequence Synthetic polynucleotide
cggatattatgctgcgatccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagtcatatactgagtataagtataatttcag

SEQ ID NO: 308 - DNA, 250 bp, Artificial Sequence
  Description of Artificial Sequence Synthetic polynucleotide
cggtaggcgcgctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagtcatatactgagtataagtataatttcag

SEQ ID NO: 309 - DNA, 1137 bp, Artificial Sequence
  Description of Artificial Sequence Synthetic polynucleotide
atgctgctgctggtcacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttctgccgccgctatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtcaagagaggccggaagaaacttctttatatattcaagcagccctttatgcgacccgttcagactacccaagaggaagatggatgcagttgccgctttccagaagaggaggagggcgggtgcgaactgtaa

SEQ ID NO: 310 - DNA, 1134 bp, Artificial Sequence
  Description of Artificial Sequence Synthetic polynucleotide
atgctgctgctggtcacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttctgccgccgctatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtcctaa

SEQ ID NO: 311 - DNA, 1470 bp, Artificial Sequence
  Description of Artificial Sequence Synthetic polynucleotide
atgctgctgctggtcacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttctgccgccgctatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaagataa

SEQ ID NO: 312 - DNA, 1347 bp, Artificial Sequence
  Description of Artificial Sequence Synthetic polynucleotide
atgctgctgctggtcacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttctgccgccgctatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtcagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaagataa

SEQ ID NO: 313 - DNA, 1461 bp, Artificial Sequence
  Description of Artificial Sequence Synthetic polynucleotide
atggctctcccggtcacagcccttctcctgcccctggcactcttgctgcatgcggcacgacccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggtggaggtggttctggcggagggggatctggtggaggcggttcagaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttctaccacaacgcccgccccgcgaccgcctactcccgctcccacaattgcatcacaacccctgtctttgagacccgaagcttgtcgaccagctgccggtggcgcggttcacacgcgggggctcgatttcgcctgtgatatatatatatgggccccattggctggaacatgcggagtattgcttctgagcctggtgattaccctctactgtaagagaggccggaagaaacttctttatatattcaagcagccctttatgcgacccgttcagactacccaagaggaagatggatgcagttgccgctttccagaagaggaggagggcgggtgcgaactgagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaagataa

SEQ ID NO: 314 - DNA, 1461 bp, Artificial Sequence
  Description of Artificial Sequence Synthetic polynucleotide
atggctctcccggtcacagcccttctcctgcccctggcactcttgctgcatgcggcacgacccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggtggaggtggttctggcggagggggatctggtggaggcggttcagaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttctaccacaacgcccgccccgcgaccgcctactcccgctcccacaattgcatcacaacccctgtctttgagacccgaagcttgtcgaccagctgccggtggcgcggttcacacgcgggggctcgatttcgcctgtgatatatatatatgggccccattggctggaacatgcggagtattgcttctgagcctggtgattaccctctactgtaagagaggccggaagaaacttctttatatattcaagcagccctttatgcgacccgttcagactacccaagaggaagatggatgcagttgccgctttccagaagaggaggagggcgggtgcgaactgagagtgaagttcagcagatccgccgatgctcccgcctataagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaagataa

SEQ ID NO: 315 - DNA, 1452 bp, Artificial Sequence
  Description of Artificial Sequence Synthetic polynucleotide
atgctgctgctggtcacatctctgctgctgtgcgagctgccccatcctgcctttctgctgatcccccaggttcaactccagcagtctggtcccggcctcgttaaaccaagccagactttgtctcttacctgtgctatcagtggcgatagcgtgtctagtaattcagccgcatggaactggatccgacaatcaccgagtaggggacttgaatggctgggtagaacctattaccggtccaaatggtacaatgactatgcagtgtctgtaaaaagcaggatcacgatcaaccctgatacgtctaaaaaccagttttctctgcaacttaatagtgtgacccctgaagacaccgctgtgtattactgtgcacgggaggttaccggtgatcttgaagatgcttttgatatatggggccaaggtacgatggtcacggtgtctagtgggggaggcggcagcgacatacagatgacgcagagcccatccagtctctccgcgtctgttggtgacagagtgactattacatgtagggcgtctcagaccatttggtcttacctcaattggtatcaacagcgaccaggcaaagcaccgaacttgctcatttacgctgccagctcactccaaagtggtgtgccgtccagatttagtggtaggggcagtggcactgatttcactctgactatttcaagtcttcaagctgaggattttgccacatactactgccagcaaagttactcaatacctcagacttttggacaggggacaaaattggagattaaatccggaaccacaacgcccgccccgcgaccgcctactcccgctcccacaattgcatcacaacccctgtctttgagacccgaagcttgtcgaccagctgccggtggcgcggttcacacgcgggggctcgatttcgcctgtgatatatatatatgggccccattggctggaacatgcggagtattgcttctgagcctggtgattaccctctactgtaagagaggccggaagaaacttctttatatattcaagcagccctttatgcgacccgttcagactacccaagaggaagatggatgcagttgccgctttccagaagaggaggagggcgggtgcgaactgagagtgaagttcagcagatccgccgatgctcccgcctataagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaagataa

SEQ ID NO: 316 - DNA, 2202 bp, Artificial Sequence
  Description of Artificial Sequence Synthetic polynucleotide
atgctgctgctggtcacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggcggcggaggatcccaggttcaactccagcagtctggtcccggcctcgttaaaccaagccagactttgtctcttacctgtgctatcagtggcgatagcgtgtctagtaattcagccgcatggaactggatccgacaatcaccgagtaggggacttgaatggctgggtagaacctattaccggtccaaatggtacaatgactatgcagtgtctgtaaaaagcaggatcacgatcaaccctgatacgtctaaaaaccagttttctctgcaacttaatagtgtgacccctgaagacaccgctgtgtattactgtgcacgggaggttaccggtgatcttgaagatgcttttgatatatggggccaaggtacgatggtcacggtgtctagtggctctacaagcggcagcggcaaacctggatctggcgagggatctaccaagggcgacatacagatgacgcagagcccatccagtctctccgcgtctgttggtgacagagtgactattacatgtagggcgtctcagaccatttggtcttacctcaattggtatcaacagcgaccaggcaaagcaccgaacttgctcatttacgctgccagctcactccaaagtggtgtgccgtccagatttagtggtaggggcagtggcactgatttcactctgactatttcaagtcttcaagctgaggattttgccacatactactgccagcaaagttactcaatacctcagacttttggacaggggacaaaattggagattaaagggggaggcggcagcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttcttccggaaccacaacgcccgccccgcgaccgcctactcccgctcccacaattgcatcacaacccctgtctttgagacccgaagcttgtcgaccagctgccggtggcgcggttcacacgcgggggctcgatttcgcctgtgatatatatatatgggccccattggctggaacatgcggagtattgcttctgagcctggtgattaccctctactgtaagagaggccggaagaaacttctttatatattcaagcagccctttatgcgacccgttcagactacccaagaggaagatggatgcagttgccgctttccagaagaggaggagggcgggtgcgaactgagagtgaagttcagcagatccgccgatgctcccgcctataagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaagataa

SEQ ID NO: 317 - DNA, 2262 bp, Artificial Sequence
  Description of Artificial Sequence Synthetic polynucleotide
atgctgctgctggtcacatctctgctgctgtgcgagctgccccatcctgcctttctgctgatcccccaggttcaactccagcagtctggtcccggcctcgttaaaccaagccagactttgtctcttacctgtgctatcagtggcgatagcgtgtctagtaattcagccgcatggaactggatccgacaatcaccgagtaggggacttgaatggctgggtagaacctattaccggtccaaatggtacaatgactatgcagtgtctgtaaaaagcaggatcacgatcaaccctgatacgtctaaaaaccagttttctctgcaacttaatagtgtgacccctgaagacaccgctgtgtattactgtgcacgggaggttaccggtgatcttgaagatgcttttgatatatggggccaaggtacgatggtcacggtgtctagtgggggaggcggcagcgacatacagatgacgcagagcccatccagtctctccgcgtctgttggtgacagagtgactattacatgtagggcgtctcagaccatttggtcttacctcaattggtatcaacagcgaccaggcaaagcaccgaacttgctcatttacgctgccagctcactccaaagtggtgtgccgtccagatttagtggtaggggcagtggcactgatttcactctgactatttcaagtcttcaagctgaggattttgccacatactactgccagcaaagttactcaatacctcagacttttggacaggggacaaaattggagattaaagggggaggcggatccggcggtggtggctccggcggtggtggttctggaggcggcggaagcggtgggggtggtagcgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttcttccggaaccacaacgcccgccccgcgaccgcctactcccgctcccacaattgcatcacaacccctgtctttgagacccgaagcttgtcgaccagctgccggtggcgcggttcacacgcgggggctcgatttcgcctgtgatatatatatatgggccccattggctggaacatgcggagtattgcttctgagcctggtgattaccctctactgtaagagaggccggaagaaacttctttatatattcaagcagccctttatgcgacccgttcagactacccaagaggaagatggatgcagttgccgctttccagaagaggaggagggcgggtgcgaactgagagtgaagttcagcagatccgccgatgctcccgcctataagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaagataa

SEQ ID NO: 318 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 319 - protein, 112 aa, Artificial Sequence
  Description of Artificial Sequence Synthetic polypeptide
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 320 - protein, 241 aa, Artificial Sequence
  Description of Artificial Sequence Synthetic polypeptide
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 321 - protein, 120 aa, Artificial Sequence
  Description of Artificial Sequence Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 322 - protein, 107 aa, Artificial Sequence
  Description of Artificial Sequence Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 323 - protein, 447 aa, Artificial Sequence
  Description of Artificial Sequence Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 324 - protein, 218 aa, Artificial Sequence
  Description of Artificial Sequence Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 325 - protein, 440 aa, Artificial Sequence
  Description of Artificial Sequence Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 326 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 327 - protein, 448 aa, Artificial Sequence
  Description of Artificial Sequence Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 214 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 329 - protein, 450 aa, Artificial Sequence
  Description of Artificial Sequence Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 330 - protein, 216 aa, Artificial Sequence
  Description of Artificial Sequence Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 331 - protein, 451 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 332 - protein, 215 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 333 - protein, 133 aa, Homo sapiens [sequence omitted]

SEQ ID NO: 334 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 335 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 336 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 337 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 338 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 339 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 340 - protein, 209 aa, Homo sapiens
RKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 341 - protein, 138 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 342 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 343 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 344 - protein, 417 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKR

SEQ ID NO: 345 - protein, 366 aa, Homo sapiens
GGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKR

SEQ ID NO: 346 - protein, 787 aa, Homo sapiens
MAVWIQAQQLQGEALHQMQALYGQHFPIEVRHYLSQWIESQAWDSVDLDNPQENIKATQLLEGLVQELQKKAEHQVGEDGFLLKIKLGHYATQLQNTYDRCPMELVRCIRHILYNEQRLVREANNGSSPAGSLADAMSQKHLQINQTFEELRLVTQDTENELKKLQQTQEYFIIQYQESLRIQAQFGPLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFESQFSVGGNELVFQVKTLSLPVVVIVHGSQDNNATATVLWDNAFAEPGRVPFAVPDKVLWPQLCEALNMKFKAEVQSNRGLTKENLVFLAQKLFNNSSSHLEDYSGLSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCESATAKAVDGYVKPQIKQVVPEFVNASADAGGGSATYMDQAPSPAVCPQAHYNMYPQNPDSVLDTDGDFDLEDTMDVARRVEELLGRPMDSQWIPHAQS

SEQ ID NO: 347 - protein, 526 aa, Homo sapiens [sequence omitted]

SEQ ID NO: 348 - DNA, 625 bp, Unknown
  Description of Unknown AP1.0 sequence
attctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattaca

SEQ ID NO: 349 - DNA, 549 bp, Unknown
  Description of Unknown CK1.0 sequence
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctgtcacg

SEQ ID NO: 350 - DNA, 630 bp, Unknown
  Description of Unknown PV1.0 sequence
aacaaaaggctacaccacttgggctacggcccgcgccaccttgtggcgcaaagacattagaagaatagcataccgcccactagggccctgcagccagcagggtaacgggcaagcacttctgtctccccggtagaacggtataggctgtacccacggccgaaaactgaactatcgttacccgactccgtacttcgcaaagcttagtaggaaactggaaagttcgagttattgacccggagtgttccccccactccagaaacgcgtgatgagggttgccaccccgaccatggcgacatggtgggcatccctgcgctggcacgcggcctctaagaggataactcgctcctactggtaaccgaagagccccgtgagctacggtttattcctccgcctccctgaatgcggctaatcctaacccatgagcagttgccatagatccatatggtggactgtcgtaacgcgtaagttgtgggcggaaccgactactttgggatggcgtgtttccttgttttctccatttgttgttgtatggtgacaagttatagatctcgatctatagcgtttcttgagagatttccaaacatttattcaagtcgtacaattcttgtgtttaagcagtacagtgtaacc

SEQ ID NO: 351 - DNA, 653 bp, Unknown
  Description of Unknown SV1.0 sequence
tctgtcctcaccccatcttcccttctttcctgcaccgttacgcttactcgcatgtgcattgagtggtgcacgtgcttgaacaaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttcgccacc

SEQ ID NO: 352 - DNA, 635 bp, Artificial Sequence
  Description of Artificial Sequence Synthetic polynucleotide
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 353 - DNA, 513 bp, Artificial Sequence
  Description of Artificial Sequence Synthetic polynucleotide
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactaaagtc

SEQ ID NO: 354 - DNA, 549 bp, Artificial Sequence
  Description of Artificial Sequence Synthetic polynucleotide
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctaaagtc

SEQ ID NO: 355 - DNA, 513 bp, Artificial Sequence
  Description of Artificial Sequence Synthetic polynucleotide
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactgccacc

SEQ ID NO: 356 - DNA, 549 bp, Artificial Sequence
  Description of Artificial Sequence Synthetic polynucleotide
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactttcactgcttttcttctcacaatccttgctcagttcaaagtc

SEQ ID NO: 357 - DNA, 678 bp, Parabovirus sp.
tgaaccgttacgcaccactcagttggtgtttggtggcaccaatgatggaacaaaaggctacaccacttgggctacggcccgcgccaccttgtggcgcaaagacattagaagaatagcataccgcccactagggccctgcagccagcagggtaacgggcaagcacttctgtctccccggtagaacggtataggctgtacccacggccgaaaactgaactatcgttacccgactccgtacttcgcaaagcttagtaggaaactggaaagttcgagttattgacccggagtgttccccccactccagaaacgcgtgatgagggttgccaccccgaccatggcgacatggtgggcatccctgcgctggcacgcggcctctaagaggataactcgctcctactggtaaccgaagagccccgtgagctacggtttattcctccgcctccctgaatgcggctaatcctaacccatgagcagttgccatagatccatatggtggactgtcgtaacgcgtaagttgtgggcggaaccgactactttgggatggcgtgtttccttgttttctccatttgttgttgtatggtgacaagttatagatctcgatctatagcgtttcttgagagatttccaaacatttattcaagtcgtacaattcttgtgtttaagcagtacagtgtaagg

SEQ ID NO: 358 - DNA, 630 bp, Artificial Sequence
  Description of Artificial Sequence Synthetic polynucleotide
aacaaaaggctacaccacttgggctacggcccgcgccaccttgtggcgcaaagacattagaagaatagcataccgcccactagggccctgcagccagcagggtaacgggcaagcacttctgtctccccggtagaacggtataggctgtacccacggccgaaaactgaactatcgttacccgactccgtacttcgcaaagcttagtaggaaactggaaagttcgagttattgacccggagtgttccccccactccagaaacgcgtgatgagggttgccaccccgaccatggcgacatggtgggcatccctgcgctggcacgcggcctctaagaggataactcgctcctactggtaaccgaagagccccgtgagctacggtttattcctccgcctccctgaatgcggctaatcctaacccatgagcagttgccatagatccatatggtggactgtcgtaacgcgtaagttgtgggcggaaccgactactttgggatggcgtgtttccttgttttctccatttgttgttgtatggtgacaagttatagatctcgatctatagcgtttcttgagagatttccaaacatttattcaagtcgtacaattcttgtgtttaagcagtacagtgtaagg

SEQ ID NO: 359 - DNA, 611 bp, Artificial Sequence
  Description of Artificial Sequence Synthetic polynucleotide
tgggctacggcccgcgccaccttgtggcgcaaagacattagaagaatagcataccgcccactagggccctgcagccagcagggtaacgggcaagcacttctgtctccccggtagaacggtataggctgtacccacggccgaaaactgaactatcgttacccgactccgtacttcgcaaagcttagtaggaaactggaaagttcgagttattgacccggagtgttccccccactccagaaacgcgtgatgagggttgccaccccgaccatggcgacatggtgggcatccctgcgctggcacgcggcctctaagaggataactcgctcctactggtaaccgaagagccccgtgagctacggtttattcctccgcctccctgaatgcggctaatcctaacccatgagcagttgccatagatccatatggtggactgtcgtaacgcgtaagttgtgggcggaaccgactactttgggatggcgtgtttccttgttttctccatttgttgttgtatggtgacaagttatagatctcgatctatagcgtttcttgagagatttccaaacatttattcaagtcgtacaattcttgtgtttaagcagtacagtgtaagg

SEQ ID NO: 360 - DNA, 618 bp, Artificial Sequence
  Description of Artificial Sequence Synthetic polynucleotide
tgaaccgttacgcaccactcagttggtgtttggtggcaccaatgatggaacaaaaggctacaccacttgggctacggcccgcgccaccttgtggcgcaaagacattagaagaatagcataccgcccactagggccctgcagccagcagggtaacgggcaagcacttctgtctccccggtagaacggtataggctgtacccacggccgaaaactgaactatcgttacccgactccgtacttcgcaaagcttagtaggaaactggaaagttcgagttattgacccggagtgttccccccactccagaaacgcgtgatgagggttgccaccccgaccatggcgacatggtgggcatccctgcgctggcacgcggcctctaagaggataactcgctcctactggtaaccgaagagccccgtgagctacggtttattcctccgcctccctgaatgcggctaatcctaacccatgagcagttgccatagatccatatggtggactgtcgtaacgcgtaagttgtgggcggaaccgactactttgggatggcgtgtttccttgttttctccatttgttgttgtatggtgacaagttatagatctcgatctatagcgtttgtaagg

SEQ ID NO: 361 - DNA, 826 bp, Unknown
  Description of Unknown Apodemus Picornavirus sequence
tttgaaaggggtgcggatatcatggcgtttctcgccatgatatccgcacattgcaaacccatattgcatacccactgggtatgcattatggggaggcccctttcacccctccccccccaattaccttttccccctctagtaaccatacgctttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattaca

SEQ ID NO: 362 - DNA, 721 bp, Artificial Sequence
  Description of Artificial Sequence Synthetic polynucleotide
cccctccccccccaattaccttttccccctctagtaaccatacgctttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattaca

SEQ ID NO: 363 - DNA, 625 bp, Artificial Sequence; identical to SEQ ID NO: 348
  Description of Artificial Sequence Synthetic polynucleotide

SEQ ID NO: 364 - DNA, 766 bp, Kobuvirus sp.
tttcacaccctcttttccggtggtccggacccagaccaccgttactccattcagctacttcggtacctgttcggaggaattaaacgggcaccctacccaagggttacatgggaccatattcctcctcccctgtaactttaagttttgtgcccgtattcttgactccaggcggatgttgtgtcgcccgtcctgtgaacaaacagctagacactttcctcccctccctctgggctgctccggcagtccactccctccccccagcgtaacatgccccgctggagtgatgcacctggaagtcgtggacgtgggttagtaacttcggtgaaaacccactataatgacaactggttgacccccacactcaaaggactcgagtctttctcccttaaggctagcccggccacatgaatttgcagctggcaactagtgagtccaccatgtcccgcaacctcggctgcggagtgctgttccccaagcgtatgccttccttctgtaagagtgcgcctggcaagcacatctgagaagtcgttccgctgcgtcgtgccaacctggcgacaggtgacccagtgtgcgtagacttcttccggattcgtccggctcttctctaggaaacatgcgtgtaaggttcatgtgccaaagccctgcgcgcggtgttcttctactgccctaggaatgtgccgcaggtacccctacttcggtagggatctgagcggtagctaattgtctacgggtagtttcatttccatcttctcttcaggtcgacatc

SEQ ID NO: 365 - DNA, 608 bp, Artificial Sequence
  Description of Artificial Sequence Synthetic polynucleotide
ttgactccaggcggatgttgtgtcgcccgtcctgtgaacaaacagctagacactttcctcccctccctctgggctgctccggcagtccactccctccccccagcgtaacatgccccgctggagtgatgcacctggaagtcgtggacgtgggttagtaacttcggtgaaaacccactataatgacaactggttgacccccacactcaaaggactcgagtctttctcccttaaggctagcccggccacatgaatttgcagctggcaactagtgagtccaccatgtcccgcaacctcggctgcggagtgctgttccccaagcgtatgccttccttctgtaagagtgcgcctggcaagcacatctgagaagtcgttccgctgcgtcgtgccaacctggcgacaggtgacccagtgtgcgtagacttcttccggattcgtccggctcttctctaggaaacatgcgtgtaaggttcatgtgccaaagccctgcgcgcggtgttcttctactgccctaggaatgtgccgcaggtacccctacttcggtagggatctgagcggtagctaattgtctacgggtagtttcatttccatcttctcttcaggtcgacatc

SEQ ID NO: 366 - DNA, 690 bp, Artificial Sequence
  Description of Artificial Sequence Synthetic polynucleotide
gaattaaacgggcaccctacccaagggttacatgggaccatattcctcctcccctgtaactttaagttttgtgcccgtattcttgactccaggcggatgttgtgtcgcccgtcctgtgaacaaacagctagacactttcctcccctccctctgggctgctccggcagtccactccctccccccagcgtaacatgccccgctggagtgatgcacctggaagtcgtggacgtgggttagtaacttcggtgaaaacccactataatgacaactggttgacccccacactcaaaggactcgagtctttctcccttaaggctagcccggccacatgaatttgcagctggcaactagtgagtccaccatgtcccgcaacctcggctgcggagtgctgttccccaagcgtatgccttccttctgtaagagtgcgcctggcaagcacatctgagaagtcgttccgctgcgtcgtgccaacctggcgacaggtgacccagtgtgcgtagacttcttccggattcgtccggctcttctctaggaaacatgcgtgtaaggttcatgtgccaaagccctgcgcgcggtgttcttctactgccctaggaatgtgccgcaggtacccctacttcggtagggatctgagcggtagctaattgtctacgggtagtttcatttccatcttctcttcaggtcgacatc

SEQ ID NO: 367 - DNA, 729 bp, Artificial Sequence
  Description of Artificial Sequence Synthetic polynucleotide
tttcacaccctcttttccggtggtccggacccagaccaccgttactccattcagctacttcggtacctgttcggaggaattaaacgggcaccctacccaagggttacatgggaccatattcctcctcccctgtaactttaagttttgtgcccgtattcttgactccaggcggatgttgtgtcgcccgtcctgtgaacaaacagctagacactttcctcccctccctctgggctgctccggcagtccactccctccccccagcgtaacatgccccgctggagtgatgcacctggaagtcgtggacgtgggttagtaacttcggtgaaaacccactataatgacaactggttgacccccacactcaaaggactcgagtctttctcccttaaggctagcccggccacatgaatttgcagctggcaactagtgagtccaccatgtcccgcaacctcggctgcggagtgctgttccccaagcgtatgccttccttctgtaagagtgcgcctggcaagcacatctgagaagtcgttccgctgcgtcgtgccaacctggcgacaggtgacccagtgtgcgtagacttcttccggattcgtccggctcttctctaggaaacatgcgtgtaaggttcatgtgccaaagccctgcgcgcggtgttcttctactgccctaggaatgtgccgcaggtacccctacttcggtagggatctgagcggtagctaattggacatc

SEQ ID NO: 368 - DNA, 650 bp, Salivirus sp.; identical to SEQ ID NO: 63

SEQ ID NO: 369 - DNA, 508 bp, Crohivirus B; identical to SEQ ID NO: 20

SEQ ID NO: 370 - DNA, 457 bp, Artificial Sequence
  Description of Artificial Sequence Synthetic polynucleotide
ccccccttttccccagccatgggttaaatggcaataaaggacgtaacaactttgtaaccattaagctttgtaattttgtaaccactaagctttgtgcacataatgtaaccatcaagcttgttagtcccagcaggaggtttgcatgcttgtagccgaaatggggctcgaccccccatagtaggatacttgattttgcattccattgtggacctgcaaactctacacatagaggctttgtcttgcatctaaacacctgagtacagtgtgtacctagaccctatagtacgggaggaccgtttgtttcctcaataaccctacataataggctaggtgggcatgcccaatttgcaagatcccagactgggggtcggtctgggcagggttagatccctgttagctactgcctgatagggtggtgctcaaccatgtgtagtttaaattgagctgttcatatacc

SEQ ID NO: 371 - DNA, 742 bp, Unknown
  Description of Unknown CVB3 sequence
ttaaaacagcctgtgggttgatcccacccacagggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagattgttaccatatagctattggattggccatccggtgaccaatagagctattatatatctctttgttgggtttataccacttagcttgaaagaggttaaaacattacaattcattgttaagttgaatacagcaaa

SEQ ID NO: 372 - DNA, 651 bp, Artificial Sequence
  Description of Artificial Sequence Synthetic polynucleotide
ttaaaacagcctgtgggttgatcccacccacagggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagattgttaccatatagctattggattggccatccggtgaagcaaa

SEQ ID NO: 373 - DNA, 721 bp, Unknown
  Description of Unknown SAFV sequence
cacttatttaattcggccttttgtgacaagcccctcggtgaaagaacctctctcttttcgacgtggttggaattgccatcatttccgacgaaagtgctatcatgcctccccgattatgtgatgttttctgccctgctgggcggagcattctcgggttgagaaaccttgaatctttttctttggaaccttggttcccccggtctaagccgcttggaatatgacagggttattttcttgatcttatttctacttttgcgggttctatccgtaaaaagggtacgtgctgccccttccttctctggagaattcacacggcggtctttccgtctctcaacaagtgtgaatgcagcatgccggaaacggtgaagaaaacagttttctgtggaaatttagagtgcacatcgaaacagctgtagcgacctcacagtagcagcggactcccctcttggcgacaagagcctctgcggccaaaagccccgtggataagatccactgctgtgagcggtgcaaccccagcaccctggttcgatgatcattctctatggaaccagaaaatggttttctcaagccctccggtagagaagccaagaatgtcctgaaggtaccccgcgtgcgggatctgatcaggagaccaattggcggtgctttacactgtcactttggtttaaaaattgtcacagcttctccaaaccaagtggtcttggttttccaattttgttga

SEQ ID NO: 374 - DNA, 675 bp, Artificial Sequence
  Description of Artificial Sequence Synthetic polynucleotide
cctctctcttttcgacgtggttggaattgccatcatttccgacgaaagtgctatcatgcctccccgattatgtgatgttttctgccctgctgggcggagcattctcgggttgagaaaccttgaatctttttctttggaaccttggttcccccggtctaagccgcttggaatatgacagggttattttcttgatcttatttctacttttgcgggttctatccgtaaaaagggtacgtgctgccccttccttctctggagaattcacacggcggtctttccgtctctcaacaagtgtgaatgcagcatgccggaaacggtgaagaaaacagttttctgtggaaatttagagtgcacatcgaaacagctgtagcgacctcacagtagcagcggactcccctcttggcgacaagagcctctgcggccaaaagccccgtggataagatccactgctgtgagcggtgcaaccccagcaccctggttcgatgatcattctctatggaaccagaaaatggttttctcaagccctccggtagagaagccaagaatgtcctgaaggtaccccgcgtgcgggatctgatcaggagaccaattggcggtgctttacactgtcactttggtttaaaaattgtcacagcttctccaaaccaagtggtcttggttttccaattttgttga

SEQ ID NO: 375 - DNA, 628 bp, Artificial Sequence
  Description of Artificial Sequence Synthetic polynucleotide
gtgctatcatgcctccccgattatgtgatgttttctgccctgctgggcggagcattctcgggttgagaaaccttgaatctttttctttggaaccttggttcccccggtctaagccgcttggaatatgacagggttattttcttgatcttatttctacttttgcgggttctatccgtaaaaagggtacgtgctgccccttccttctctggagaattcacacggcggtctttccgtctctcaacaagtgtgaatgcagcatgccggaaacggtgaagaaaacagttttctgtggaaatttagagtgcacatcgaaacagctgtagcgacctcacagtagcagcggactcccctcttggcgacaagagcctctgcggccaaaagccccgtggataagatccactgctgtgagcggtgcaaccccagcaccctggttcgatgatcattctctatggaaccagaaaatggttttctcaagccctccggtagagaagccaagaatgtcctgaaggtaccccgcgtgcgggatctgatcaggagaccaattggcggtgctttacactgtcactttggtttaaaaattgtcacagcttctccaaaccaagtggtcttggttttccaattttgttga

SEQ ID NO: 376 - DNA, 674 bp, Artificial Sequence
  Description of Artificial Sequence Synthetic polynucleotide
cacttatttaattcggccttttgtgacaagcccctcggtgaaagaacctctctcttttcgacgtggttggaattgccatcatttccgacgaaagtgctatcatgcctccccgattatgtgatgttttctgccctgctgggcggagcattctcgggttgagaaaccttgaatctttttctttggaaccttggttcccccggtctaagccgcttggaatatgacagggttattttcttgatcttatttctacttttgcgggttctatccgtaaaaagggtacgtgctgccccttccttctctggagaattcacacggcggtctttccgtctctcaacaagtgtgaatgcagcatgccggaaacggtgaagaaaacagttttctgtggaaatttagagtgcacatcgaaacagctgtagcgacctcacagtagcagcggactcccctcttggcgacaagagcctctgcggccaaaagccccgtggataagatccactgctgtgagcggtgcaaccccagcaccctggttcgatgatcattctctatggaaccagaaaatggttttctcaagccctccggtagagaagccaagaatgtcctgaaggtaccccgcgtgcgggatctgatcaggagaccaattggcggtgctttacactgtcactttggtttaatgttga

SEQ ID NO: 377 - DNA, 726 bp, Artificial Sequence
  Description of Artificial Sequence Synthetic polynucleotide
cacttatttaattcggccttttgtgacaagcccctcggtgaaagaacctctctcttttcgacgtggttggaattgccatcatttccgacgaaagtgctatcatgcctccccgattatgtgatgttttctgccctgctgggcggagcattctcgggttgagaaaccttgaatctttttctttggaaccttggttcccccggtctaagccgcttggaatatgacagggttattttcttgatcttatttctacttttgcgggttctatccgtaaaaagggtacgtgctgccccttccttctctggagaattcacacggcggtctttccgtctctcaacaagtgtgaatgcagcatgccggaaacggtgaagaaaacagttttctgtggaaatttagagtgcacatcgaaacagctgtagcgacctcacagtagcagcggactcccctcttggcgacaagagcctctgcggccaaaagccccgtggataagatccactgctgtgagcggtgcaaccccagcaccctggttcgatgatcattctctatggaaccagaaaatggttttctcaagccctccggtagagaagccaagaatgtcctgaaggtaccccgcgtgcgggatctgatcaggagaccaattggcggtgctttacactgtcactttggtttaaaaattgtcacagcttctccaaaccaagtggtcttggttttccaattttgttgaccgcc

SEQ ID NO: 378 - DNA, 549 bp, Unknown
  Description of Unknown GLuc CK dCTG1 sequence
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctacgtctgacaactcactgactatccacttgctctaaagtc

SEQ ID NO: 379 - DNA, 549 bp, Unknown
  Description of Unknown GLuc CK dCTG1_2 sequence
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctacgtcgtacaactcactgactatccacttgctctaaagtc

SEQ ID NO: 380 - DNA, 549 bp, Unknown
  Description of Unknown GLuc CK dCTG1_2_3 sequence
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctacgtcgtacaactcacgtactatccacttgctctaaagtc

SEQ ID NO: 381 - DNA, 549 bp, Unknown
  Description of Unknown GLuc CK dAll sequence
gtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctacgtcgtacaactcacgtactactcactgtctctaaagtc

SEQ ID NO: 382 - DNA, 549 bp, Unknown
  Description of Unknown CK SZ1-L1S sequence
gggggtggggggggcctcggccccctcaccctcttttccggtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgaaccacccaggaatgccagggaggtaccccgcttcacagcgggatctgaccctgggctaattgtctacggtggttcttcttgcttccacttctttctactgttcgccacc

SEQ ID NO: 383 - DNA, 553 bp, Unknown
  Description of Unknown CK Aichi Scan (AV-S) sequence
gggggtggggggggcctcggccccctcaccctcttttccggtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcatttccaatccttttatgtcggagtc

SEQ ID NO: 384 - DNA, 667 bp, Unknown
  Description of Unknown CK Aichi Loop (AV-L1) sequence
gggggtggggggggcctcggccccctcaccctcttttccggtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgaactgccctaggaatgccaggcaggtaccccacctccgggtgggatctgagcctgggctaattgtctacgggtagttttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 385 - DNA, 693 bp, Unknown
  Description of Unknown CK SZ1-L2 sequence
gggggtggggggggcctcggccccctcaccctcttttccggtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcctgatcccccgcggaagctgctcacgtggcaactgtggggacccagacaggttatcaaaggcacccggtctttccgccttcaggagtatccctgctagtgaattctagtagggctctgcttgcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 386 - DNA, 671 bp, Unknown
  Description of Unknown CK Aichi TriLoop (AV-L2) sequence
gggggtggggggggcctcggccccctcaccctcttttccggtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcatgtgcctggcaagcatatctgagaaggtgttccgctgtggctgccaacctggtgacaggtgccccagtgtgcgtaaccttcttccgtctccggacggtgcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 387 - DNA, 523 bp, Artificial Sequence
  Description of Artificial Sequence Synthetic polynucleotide
gggggtggggggggcctcggccccctcaccctcttttccggtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtg

SEQ ID NO: 388 - DNA, 592 bp, Artificial Sequence
  Description of Artificial Sequence Synthetic polynucleotide
gggggtggggggggcctcggccccctcaccctcttttccggtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgatttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 389 - DNA, 572 bp, Artificial Sequence
  Description of Artificial Sequence Synthetic polynucleotide
gggggtggggggggcctcggccccctcaccctcttttccggtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 390 - DNA, 167 bp, Artificial Sequence
  Description of Artificial Sequence Synthetic polynucleotide
ccctgcagccgtcaccgtaagtttgaagttaccgcatatcagcctctgcttcccagcgcgtccaattcctgttcttattgtttcccctccaggcgttacgcgtgacgacgaactgtgtcgcagctaccacattattccggagccttcattctcgcggctctgatcgt

SEQ ID NO: 391 - DNA, 43 bp, Artificial Sequence
  Description of Artificial Sequence Synthetic oligonucleotide
ggagaccgcggccacgccgagtaggatcgagggtacagtctcc

SEQ ID NO: 392 - DNA, 167 bp, Artificial Sequence
  Description of Artificial Sequence Synthetic polynucleotide
gacaccaggatcactcttgctctgacccgccctgtgtagaatagactcatgcttccctaagacctggatttcttcccaggcactttcacccgcctgccctgctccttcagtggactgcacccagggaggcggtctctgactgtcctttactttctattctggattgc

SEQ ID NO: 393 - DNA, 30 bp, Artificial Sequence
  Description of Artificial Sequence Synthetic oligonucleotide
aaacccccctaagccgccgccgccgccacc

SEQ ID NO: 394 - DNA, 18 bp, Artificial Sequence
  Description of Artificial Sequence Synthetic oligonucleotide
cccccccaacccgtcacg

SEQ ID NO: 395 - DNA, 6 bp, Artificial Sequence
  Description of Artificial Sequence Synthetic oligonucleotide
gtcacg

SEQ ID NO: 396 - DNA, 141 bp, Artificial Sequence
  Description of Artificial Sequence Synthetic polynucleotide
tctgcgcactcgtaatcagtactaacccccctttgtcggacactatgcgataatcgatccgcctttttcaccgccttcggaattttatttacctcaactgatcctggagtctctcttggttttcacggaggcctccgccca

SEQ ID NO: 397 - DNA, 43 bp, Artificial Sequence; identical to SEQ ID NO: 391
  Description of Artificial Sequence Synthetic oligonucleotide

SEQ ID NO: 398 - DNA, 141 bp, Artificial Sequence
  Description of Artificial Sequence Synthetic polynucleotide
ccccttgaaacccccgccccaggttcagtctctcttcatccctctgtcctgcatggtgatacaaagaccctttgtggaccctaagccatgtagttgctgctccctccttccagttgtgaatattggtttctgttaatcaca

SEQ ID NO: 399 - DNA, 30 bp, Artificial Sequence; identical to SEQ ID NO: 393
  Description of Artificial Sequence Synthetic oligonucleotide

SEQ ID NO: 400 - DNA, 18 bp, Artificial Sequence
  Description of Artificial Sequence Synthetic oligonucleotide
cccccccaacccgtcacg

SEQ ID NO: 401 - DNA, 6 bp, Artificial Sequence
  Description of Artificial Sequence Synthetic oligonucleotide
gtcacg

SEQ ID NO: 402 - DNA, 6 bp, Artificial Sequence
  Description of Artificial Sequence Synthetic oligonucleotide
gtcacg

SEQ ID NO: 403 - DNA, 43 bp, Artificial Sequence
  Description of Artificial Sequence Synthetic oligonucleotide
aataagagagaaaagaagagtaagaagaaatataagagccacc

SEQ ID NO: 404 - DNA, 49 bp, Artificial Sequence
  Description of Artificial Sequence Synthetic oligonucleotide
cgaactagtattcttctggtccccacagactcagagagaacccgccacc

SEQ ID NO: 405 - DNA, 7 bp, Artificial Sequence
  Description of Artificial Sequence Synthetic oligonucleotide
agccacc

SEQ ID NO: 406 - DNA, 14 bp, Artificial Sequence
  Description of Artificial Sequence Synthetic oligonucleotide
tgatagctaactag

SEQ ID NO: 407 - DNA, 14 bp, Artificial Sequence
  Description of Artificial Sequence Synthetic oligonucleotide
tagtagctaactag

SEQ ID NO: 408 - DNA, 14 bp, Artificial Sequence
  Description of Artificial Sequence Synthetic oligonucleotide
tgatgactgagtga

SEQ ID NO: 409 - DNA, 14 bp, Artificial Sequence
  Description of Artificial Sequence Synthetic oligonucleotide
tagtagctaggtag

SEQ ID NO: 410 - DNA, 3 bp, Artificial Sequence
  Description of Artificial Sequence Synthetic oligonucleotide
taa

SEQ ID NO: 411 - DNA, 14 bp, Artificial Sequence
  Description of Artificial Sequence Synthetic oligonucleotide
taatagctaactag

SEQ ID NO: 412 - DNA, 15 bp, Artificial Sequence
  Description of Artificial Sequence Synthetic oligonucleotide
taactagctaactag

SEQ ID NO: 413 - DNA, 579 bp, Rhopalosiphum padi virus
gataaaagaacctataatcccttcgcacaccgcgtcacaccgcgctatatgctgctcattaggaattacggctccttttttgtggatacaatctcttgtatacgatatacttattgttaatttcattgacctttacgcaatcctgcgtaaatgctggtatagggtgtacttcggatttccgagcctatattggttttgaaaggacctttaagtccctactatactacattgtactagcgtaggccacgtaggcccgtaagatattataactattttattatattttattcaccccccacattaatcccagttaaagctttataactataagtaagccgtgccgaaacgttaatcggtcgctagttgcgtaacaactgttagtttaattttccaaaatttatttttcacaatttttagttaagattttagcttgccttaagcagtctttatatcttctgtatattattttaaagtttataggagcaaagttcgctttactcgcaatagctattttatttattttaggaatattatcacctcgtaattatttaattataacattagctttatctatttata

SEQ ID NO: 414 - DNA, 675 bp, Artificial Sequence
  Description of Artificial Sequence Synthetic polynucleotide
cttgattctaaccttgccgtatggtgccctaacgggttcatttaatcatgcgatgagggttgctataccgcatccattctaaggcgattcaatgcttcatttaggaattttgttgacgattaaaaggtacccccacaaaaacaaaaccaatcttacttgattttcgttttaactgaccactgcgatcccaaattttcgccttcttatcaaagtatgttgtgttctttgggtgtacaacctgagaacttgtctacaactacatattactcgaggaagaaattcggtttaagccgtgccttctcacgtttagtatatctatctggacacaccttcttcatcttctaatccccatctagtctcctgatcagagacgtcgttattaacaaataaccccccttgttaataagagacaaagtacaatcaagctaagttctcttggagttcctgtaggaacttagccattgtgatagagtcataagtctatgtgcatagacagctctagctcaccatttccttcccaacccatcttttcatcagcttaactctatgaatccgatgcaaaaaccattctaacatcttatggtgctttccaagccaaatgagagctcactcttttgagccgctatttaatggacaataaacgttttatagtgtacatcatattgtaaaaacaaa

SEQ ID NO: 415 - DNA, 636 bp, Oscivirus sp.
cctcggtccctctttccgtcgccgcccacgacgttaaatgcggtgttgtggtgcttaggtgccacaccactgctatttgggtccccctttcccctatatatgtttgtttgtttatttcaatttcttgaggattggcacctccttatgccaaatctaaatcgtggaggatcccaggctttctggtctttaacagaactccacgtccaggtcatagaaactggttggtaggctgcctgagtagtccatttgctagtagtcccttgtgaacagggtggctcccgtttactgctggtattcccggtgtaggtcgccatggtggtaacaccatcctgcattgtgtgtgaaccagtaccgcaaggatagcaaggtatgaacacttgtggacgaaatggtaagtgatcaattcactttcatggccggaaggtcacgtggcaatcatgccacccaggtaccctcctctgggaggatctgagggtgggctaagcagaccctgccatgtggctgaacttttcccttattgttttactttgtaacatttatagttgtgttagtgatttgtgtgttgtgcccttgtgagctatatccagtataagttcgcagctagaagttaatccttcgacatcggctgtattggaa

SEQ ID NO: 416 - DNA, 675 bp, Cadicivirus B
caccaacccttgacctgtaatgtcagtggacagagtgctcctctgttcccggttaccgtgttccaggacacgattgtaatcctgcgcctcaccagcgctgcgtgcacgtctgcataaggaaacgtgccttccccatgtctctatcaattctttggtgagtgaccgccctagttgctcatcctatgggattcttctctcatgggttctttgtggcatgcgaatgtcaccttaattggaggtctttaattagatatcctttcttcatctttgatatgagtgtcggaatttgattcctagtctctgcaaaacaaccccacttgatgaattcaacttttcaaccgcacaaacataatcaggtttttaaattgaatgtttctaaattctaaatttagtttatttaagtagtttgccatcttgactcgatgtaaaattgtcatacaagtcttcttttcttttctttacactttgaagtttgcacttagcagtcgttctgcacagctttcgagttttgtttgatcgacatcgcaacttccacccacctctctttttctagtgttgaatgcggctaatcctaacccgagagcaataaacccaggtttattgtcgtaacgcgcaagtcttggacggaaccgactatacacacacctctttaccctttagtacacccttggtacg

SEQ ID NO: 417 - DNA, 570 bp, Artificial Sequence
  Description of Artificial Sequence Synthetic polynucleotide
gcaaaatgggtacgtagttaaccactgcgtatcaggattgcaggccacgaagggtatttgcatatctttctatgcggtattacggcttaaaacccgttgtatcttgttgtttgactgcctgtatcactagtggccattttatttaggttagagacccctgatagtaggagagttacaaactctttaaaaattgttgaccccggaaaagatggtgacccctgtaagtagttgatcaagaagatctatgcgctggcatagtaatccagtgtttcctgttttaggatgacctctgaaagtagatgaccgtggaaagtcacgtagtgccccaataagcacgtttgggcagcgtgcgctatcacaaggcttgatctccgaggagccccttgttttagctggctggaagccaatgatcttaagtagataagtgctgttgcttgtagttcaacagaaagctttgagtacgtctttcttgcgagaaagaacacatgcattcttatgctctcaattctattatttttattttgggcgaaaggaaagctctcacgcgagtacgaatagccaaccctttat

SEQ ID NO: 418 - DNA, 145 bp, Plautia stali intestine virus
gctgactatgtgatcttattaaaattaggttaaatttcgaggttaaaaatagttttaatattgctatagtcttagaggtcttgtatatttatacttaccacacaagatggaccggagcagccctccaatatctagtgtaccctcg

SEQ ID NO: 419 - DNA, 312 bp, Unknown
  Description of Unknown PV Mahoney sequence
atgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggcc

SEQ ID NO: 420 - DNA, 577 bp, Reticuloendotheliosis virus
ggggtcgccgtcctacacattgttgtgacgtgcggcccagattcgaatctgtaataaaagctttttcttctatatcctcagattggcagtgagaggagattttgttcgtggtgttggctggcctactgggtggggtagggatccggactgaatccgtagtatttcggtacaacatttgggggctcgtccgggattcctccccatcggcagaggtgcctactgtttcttcgaactccggcgccggtaagtaagtacttgattttggtacctcgcgagggtttgggaggatcggagtggcgggacgctgccgggaagctccacctccgctcagcaggggacgccctggtctgagctctgtggtatctgattgttgttgaaccgtctctaagacggtgatactataagtcgtggtttgtgtgtttgtttgttaccttgtgtttgttcgtcacttgtcgacagcgccctgcgaattggtgtacccacaccgcgcggcttgcgaataatactttggagagtcttttgcctccagtgtcttccgtttgtactcgtcctcctctccctctccggccgggatggg

SEQ ID NO: 421 - DNA, 675 bp, Tropivirus A
tgtcgcatgttgccaacatcaaaattctgggagagtcgcgaactccttaacactgccttgcctcgacggagccgttgttatagtgtcgacgggatacaaacattaaactaaacccacttgcctcgacggaaccccttaccttttattttattttatagtatgaaagtgaatcttgtatgaatgttcatagaaaactgcaaatgagtaccacgtctaacatgagagaatgatactggagaaatccaagtttagaagtcactacgaatcccagcggaaacaagggaattctgagcttctaataggcgtttaagactatttgcaaaattctggtgcgtaagtgatattttcattgcgtagaacgctggtaaccactccggctagtataagcattgttagtcacttattatgaaactccacactatcctttctggagaagcacacaaacttacatggtaaagctagaccattatcttaagcggtgagtacactgcaaccttgtaacaatgcttgtatgactactttttgtatatcttgagcaatattgttgaggtggacatgtccaaaggtaatgttgttgggaatggaggggtccattttcccgtgcacgtagtgtactagtattgggtgatagccttgcggcggatcaaccatgtattttaatccgttgactttcac

SEQ ID NO: 422 - DNA, 592 bp, Symapivirus A
ttgggaaatccccaatgcttctttcaacaccgcctgactatgcggtggcgcttcggctcaaacaactagtcacttccccctcttaactactacccaagacttctaactacccttacctacttatttgtctaaatttcaaacttttattctcacgcgtcttataaacatcttttctatttgttatggtatgttttgtgatttgtgtggtgtatttcatttaatgggatctagtggaccgtgccccggttgggtatccgctccctttaaatgtttgcaagcactcttgacattataacctatcatttagtttacttgtttgtatgatcgtatttctgaatcgtaacatttatgcaattctttctcgccgagacttgtctaggagataaagttcctgcatatttagtgttacggttgtataatggagacttagatagcttcacactgaggacgctttttcgctatccttttgacctgattcaggccagtgtggagttaatgattgtatggatgggccctacaatttgtctaagacttggtgatagcctcgcggccgctcgccatttatacaactgaatagcggttgaaactctct

SEQ ID NO: 423 - DNA, 591 bp, Artificial Sequence
  Description of Artificial Sequence Synthetic polynucleotide
tcacgcgcttttccggtggtcacccaccgttagggagcgccagcgttcgcgcttccgctaccaggtgacacactcctttcccctcccccattcccgttcccatcctctggactggtttctcctcacgattgaccagcagctgggagctgttaccagacgttggacagtaagtcccggatgcactatagggctggtggctagtgcttggtaagcactcaacgccatacctaatgtgtacctcggcttgccctcctggtcgtggtgaccggctgtttctcttcccttggctccagacgggctggtgtcctaccaccaccgttgcatgcagacctccccctgcgcactcgaacgccctgtcccagcagggttagtatgtgctgtgcagatctgcatgtgacaccccatccactggtagagcaggaagttgccctagctaacgcggcaagtattactttccgctacacgtccttgagattcctcggacctctggaactagggtgactgtgggcttgggaaaacccaccttggtcctgtactgcctgatagggtcgcggctggccgaccagtggatgtagccagttgttttgggat

SEQ ID NO: 424 - DNA, 538 bp, Rosavirus C
cagggagatctccatgaataatcttttccaccctctttagcgtctatgctattgaggacgggttggagccccgttgacccagcgtcagagtgtgtcggtagcaggctttctgctctcgccccatgccggccacacctcccattagtgatgtgaaggttgtaagttacatgtgaaaaggtttctaataattgagctgaatgtagcgattacctaaggtgagcggattcccccacgtggtaacacgtgcctctcaggccaaaagccaaggtgttaaaagcaccccttaggtaggccactaccccgtggcctcagttctcttagaagattcacttagtagtgtgtgcactggcaactcttaagcagagctagtgagtgggctaaggatgccctgaaggtacccgcaggtaacgttaagacactgtggatctgatcaggggctcgagtgctgaagctttacagaggtagctcgagttaaaaaacgtctatgcccctcccccacgggagtgggggttcccccacaccaattttagattgcact

SEQ ID NO: 425 - DNA, 675 bp, Rosavirus sp.
ccaggcatggcgttaaacatgcattcccttcccctagtaacctcccttcgccccttccccacgttgtaccccctccgagatggctgctaaggcgcttgctgctacagcagtctcgtgtttcgggtgttataagtgctttcttttccactccactccctgcctatggggagcggaacggccttgtctcggtcgttgcttcttgcagatcttcacccctccaggctttctggactcgccaggggtggagtagtaggcgcactgtctaagtgaaggtagcagtgttgttggcgaagagttgtggacctactttgagtttgtagcgatcatccagagctagcggatctccccacgcggtaacgcgtgcctctaggcccaaaaggcacggtgttcacagcaccctttggatggcgggggtgcccccctccgcacttaaagtagaaaaacagcttagtagtcaaataacatggctttcctcaagcattcagtgctacatgggactgaaggatgcccagaaggtacccgcaggcaacgataagctcactgtggatctgatctggggccctgggccaggtgctatacacctggttaaaaccaaatctggtagtcagggttaaaaaacgtctaagtcccacccccccggggacggggggttcccttaaaccctcaactgacacc

SEQ ID NO: 426 - DNA, 656 bp, Unknown
  Description of Unknown Rhimavirus A sequence
cgaattccggacatctcctttcgggggcgagcgtcaccgtgcccctcatggaggcaactgtgcctctaatcggtgacccactgagaaaattttctttctacgtggctaaacaatgcaactttataataacacaaatttaatgcttaatcttaacaccaaagatttgaacatatgtttggaaagtggcacacttcaaacattgcatagttgctaggggtgaagtccctttaaggggttgcagaggatctttcctctttatgagcggctaggagtatcttcttgatattatgtggtcgtgcaactcacttcccagatgtatgacggtgtactaagcgattggaactagtcataacctctttgaattttggtattgcgagtctagcagggggatatttaccgctaaagggtgacacactcgtgagggtggcctttggtgtgtgtatatttattccgcccatcttgcatggggtgctaaaattctaatgctgtgaaataaccattttctgaatacattctctacatttggagtcaaatatgaggaatgccactcaggtacccttgacatgatcttggatctgagagtgggctaattatctaattatttggcgactttctaaaatcttctgtttttagtggtgacaatttatggttataaa

SEQ ID NO: 427 - DNA, 487 bp, Rafivirus sp.
gtgtccgggaagcgactcaagcttttgactgagtctctacaccttcatccgtaacatctttaagtttatgtgcctatggacctctagtgcactgccatcaccgggggtgtattggactggtttttccacaatccattcatcctgaggaattttggctttgttactaggatggtcccaccacacgcttatctgtgcctattgtgtcaaccatgttcttaagtagttgtgcccgtgggtgagtagataaccacaacaatccgataaagcatctcgcaaggatgtgagtaatggagtgtatgtgctacagagacccacaacctgaaccaagagagacacagtgaggattgtaaagggggaactctttgaaagggcatgtcccgcaattcctactgactgacaccgggggttggtgtcggtggattttagcaaatcctgttactgggtgatagccttgtgcacttcacttggttcttgtataagtgctgta

SEQ ID NO: 428 - DNA, 560 bp, Rafivirus sp.
tgcgaatttattcgcacagtctcttttcccccatcttgtgtgtgtgatggggtaagccgcagagtaatacctactctgctgcaaacacactcactcttttctatctactttatatcatgtaataataagtagggaacatattcaattcatattgttcatctcactgaacccgcatgaaggactgcattgcatatcctggacgaagtgacgtggaatatttggacatttatggattggacaccattacgctttgtgcctctacggagatgtaaccataatcttaagtagtagtaccccagcacaagaggataaagtggcatacacgacaacgggtgttgctcgcaccttagtaatgtggatgttcacccttggagcgtgctgaaactctgtgggtaaagacacacattagtacaaatgtgggggaactcactgaaagggcatgtcccgtgtactggtgtgccggaaagtgggggtcgctttctggagaacttagtagttcttgttattgggtgatagccttgcggcggatcaactcacagttttaatccgttgttttgcat

SEQ ID NO: 429 - DNA, 675 bp, Poecivirus sp.
actacacaatcgcaacacgcgcaagtttgtagtttgattggcgtgcaaatgtcaaatcaagcatataacacaatttggtggctgttggtgtttgttataggaattttggttgtgttgaaattgtggatgtgtaggaaatatgcacaattacgtcagcgtcaggagttttataacctggcgcaacaccaaaatggtcttcgcgctttaacatcaccagcgaggtgtaaacaaattgaagttgaattagatcgtgtataggccagggaaccatccctcccaacgccacatcttgtggggaagttgggataatggtgggtctatatgaattggtctgtagacccacagtgaagagtgaatagtatgcttgcggttccatttgttaatggtctagcatgggtgggggcggcaaccccgtgaggggttccccactggccaaaagcccaggggttagtcatttcaaccaaggaagctggtaacctggtgacctgaacttgagtggtgagacccccttgctagagtgtgtaaaccgattgtaagcattttgtttgcttagtatctgtggtataagcagtcaattttgtataggctcaaggctgtggtagttagtagatgcccggaaggttattactgatccggggaccgtgactatacattaggtaaaccggtttaaaaacc

SEQ ID NO: 430 - DNA, 519 bp, Unknown
  Description of Unknown Megirivirus A LY sequence
ttcgggacactggatgggcgacttggtggggctgccactctatcttgacctttcgttactgactttcggatctctgactcctccttgtctcttgcgtttggtccacggacggactaattggaatgtttactggctaagcctcgttctgaaataccctagccaatgggttgtagtaggatcctggtgtttccattaaacctcttccgaccatagtagctagagttatggctgtgtaggatgtgggtaagaccgctttttgcgtatctcccacaagacaccggattatggatgtgtccgctggataaggctcgaaacctcccaactgaaggtggtgctgaaatattgcaagcctaggttgtgtagaggcaagtagatgcctgccgcgacattcgtcttccgcccttttgggttagtagtgtacctacatggacgtggggctgggaatccccaccttgcataacactggttgatagacctgcggctggtcaagttactatggtataaccagttgaaatggct

SEQ ID NO: 431 - DNA, 675 bp, Unknown
  Description of Unknown Megirivirus E sequence
gcttggcaacctcatatcgttactctgccgaccagtctgggtcgtgtggccacacaatgggattcgttctgttgtgtagagtcacatggcattactgggctgatcggtggggatccgttgccacccctaaacccttacatttactggactgcttttcttggccccggaatgattcgctcacccgcgatgaggactgttgttcttattatggcaggattacgcgtctggtccgcgtaaggactaattcctatgtttatacgttactaccttgttctgaacggtgggcgccaccccgcctagtaggatcctggcttatcgtgtagacctctagggaccacattagctagagtgtaggctgctatggatggagtagtgacccctttttgggtatcactctctaagactccggaatgtgtcatagtacgctggaaatccttacttgtttttccatgagggggaggtggtgctgaaatattgcaagccacccctcggttaaaacagtttggtgccgcttatgccatattaccgccccttgtagttgggctgtttttgcagctccgggttagtagagtaccatagtggacgcggtgttgggaatcaccgccttggctgcacactgcttgatagagctgcggctggtcaagctaattgtggtataaccagttgatttggcat

SEQ ID NO: 432 - DNA, 639 bp, Unknown
  Description of Unknown Megirivirus C sequence
ttcccgaccggtctggcaaaccggacggttatcctggttagatgtctgatggttgctggaacgtggtggctactgctgccaccttctggcttcctttaatgggcatctagctgggttctttgccacaatccatcttactctcttacccattttctattacccagacttgttgttaactggtaaagttgacctactggcttcgttttgagactattctggtgttggtggacactctttccacaagtagattgtatggagttcatgctcgttttgaaccgggaatggcacaacccgtagtaggatcttgcctctgccatactaatctgcgcctgttgcttttagactatgggctgctaaggatgacattggaacccctttttggatattccatgtcaagtcaactgtttcatctggtgtacgctggaaatccttgttccgaggtcttgtctggaggtggtgctgaaatattgcaagccacaggcagttccttggacttggtgccgctatcagatgctacaccctctatgggcaaatgttgaaccttagtggacgcgtgagatgggaatccacgccggccatagactggctgataagctcgcggctgatcgagttgcaacagtaatcagttgatttgccact

SEQ ID NO: 433 - DNA, 497 bp, Ludopivirus sp.
tagacccccacctagcccttttccccgtcagtggggggcttactcactgggcatctgttaatctggcctaactagattgacaccactcccttggaacgtaactccacgctaactcactggctctacgcacagacacacggtctttctgctatccccggggaagataccagatggcgaccggctgtcccagcggcctagtagctactcgggttgagtacccaccacggttttgacgcctgctaaaattcaagagacagaggtaggggtgcttagtgtgtgggggaagttcccacaagcgaggcaaagcattgctccctcgcgtcaccgggtgcaaggtaaattggctggacttccgctctacccttgctactcgccctcttcggagggttcgaagtgacactaggtatacgcatggttgggaaaccatgcctggcctactactgggtgatagcctggcggcgggtccgtctcttggcttatacccgttgatttgggat

SEQ ID NO: 434 - DNA, 610 bp, Livupivirus sp.
tatctacatggggatccaggctgtatggaatgtctgtcttaacaagcactataccagaaagatccacccaaagtggtgggactgggactgtgaggtgagaaatcccgaaaccagccttctcaagcgtcggacgatctttctgttttagtgaacaccttgccttttaaatggatgacaacaccccttcagcaaatcgcaatctgaaatcccaaaagactgtttagccgaactctggtaatcactccggagaagtaggatacgcagcccctgtggactcttgatttcaggactcaaggtagctagagctggaacttcatggaatgacaaaggaatatatgcacattgtgcgctttcctggccttgtagcccgtcgtgaggatatgtcgttgggaatcgacatcttagtccagtactgcttgatagagtgtcggctggcacagttacctgagaataagtcagttgtacttaacatgaacaaaaaaaataactaccacaactaccacaatctaccaatacttgaattatgctgaatctcgtacagtaaaaacgttccgtggaaggacaagtattgaagtgcggttacatcatccgatacgcgctggatccctca

SEQ ID NO: 435 - DNA, 629 bp, Aichivirus A
cacccatacacccccacccccttttctgtaactcaagtatgtgtgctcgtaatcttgactcccacggaatggatcgatccgctggagaacaaactgctagatccacatcctccctccccttgggaggacctcggtcctcccacatcctccctccagcctgacgtatcacaggctgtgtgaagcccccgcgaaagctgctcacgtggcaattgtgggtccccccttcatcaagacaccaggtctttcctccttaaggctagccccgatgtgtgaattcacattgggcaactagtggtgtcactgtgcgctcccaatctcggccgcggagtgctgttccccaagccaaacccctggcccttcactatgtgcctggcaagcatatctgagaaggtgttccgctgtggctgccagcctggtaacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccaccttcgggtgggatctgagcctgggctaattgtctacgggtagtttcatttccaattcttttatgctggagtc

SEQ ID NO: 436 - DNA, 675 bp, Aichivirus sp.
tactccattcagcttcttcggaacctgttcggaggaattaaacgggcacccatactcccccccaccccccttttgtaactaagtatgtgtgctcgtgaccttgactcccacggaacggaccgatccgttggtgaacaaacagctaggtccacatcctcctttcccctgggagggtccccgccctcccacatcccccccccagcctgacgtgtcacaggctgtgtgaagcccccgcgaaagctgctcacgtggcaattgtgggtccccccttcatcaagacaccaggtctttcctccttaaggctagccccggcgtgtgaactcacgttgggcaactagtggtgtcactgtgcgctcccaatctcggccgcggagtgctgttccccaagccaaacccctggcccttcactatgtgcctggcaagcacacctgagaaggtgttccgctgtggctgccagcctggtaacaggtgccccagtgtgcgtaaccttcttccgtcttcggacggtggtgattggttaagatttggtgtaaggttcatgtgccaacgccctgtgcgggatgaaacctctactgccctaggaatgccaggcaggtaccccaccttcgggtgggatctgagcctgggctaattgtctacgggtggtttcatttccaattctttcatgtcggagtc

SEQ ID NO: 437 - DNA, 675 bp, Aichivirus sp.
tactccattcagcttcttcggaacctgttcggaggaattaaacgggcacccatacacccccacccccttttctgcaacttaagtatgtgtgctcgtaatcttgactcccacggaacggatcgatccgctggagaacaaactgctagatccacatcctcccttcccctgggaggaccccggtcctcccacatcctccccccagcctgacgtaacacaggctgtgtgaagtccccgcgaaagctgctcacgtggcaattgtgggtccccccttcaccaagacaccaggtctttcctccttaaggctagccccgatgtgtgaattcacattgggcaactagtggtgtcactgtgcgctcccaatctcggccgcggagtgctgttccccaagccaaacccctggcccttcactatgtgcctggcaagcatatctgagaaggtgttccgctgtggctgccagcctggtaacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccaccttcgggtgggatctgagcctgggctaattgtctacgggtagtttcatttccaattcttttatgtcggagtc

SEQ ID NO: 438 - DNA, 610 bp, Kobuvirus sp.
gtaacttcaagtgtgtgtgctcgtaatcttgactcctgccggaatgccgcccggttcagtgaacaaacagctaggcaagtccctcccttcccctgtggtcggttctcaccggccaccatccctcccccagcctgacgtgttacaggctgtgcaaagcccccgcgaaagctgctcacgtggcaattgtgggtcccccctttgtcaagacaccgagtctttctcccttaaggctagcccggtcccacgaacgtggaactggcaactagtggtgtcactacacgcctccgacctcggacgcggagtgctgttccccaagctgtaaccctgacccaagactgtgctgcctggcaagcaccgtctgggaagatgttccgctgtggctgccaaacctggtaacaggtgccccagtgtgtgtagtcttcctccagtctccggactggcagtcttgtgtaaagatgcagtgtaaggttcaagtgccaaatccctggaaggagtgaccctctactgccctaggaatgctgtgcaggtacccccaacttcggttggggatctgagcacaggctaattgtctacgggtagtttcatttcccatcctctcttttttggcatc

SEQ ID NO: 439 - DNA, 576 bp, Kobuvirus sp.
tttgaaaagggggtgggggggcctcggccccctcaccctcttttccggtggccacccgcccgggccaccgttactccactccactccttcgggactggtttggaggaacataacagggcttcccatccctgtttacccttactccactcacccctccccttgaccaaccctatccacaccccactgactgactcctttggatcttgacctcggaatgcctacttgacctcccacttgcctctcccttttcggattgccggtggtgcctggcggaaaaagcacaagtgtgttgttggctaccaaactcctacccgacaaaggtgcgtgtccgcgtgctgagtaatgggataggagatgccaataacaggctcgcccatgagtagagcatggactgcggtgcatgtgacttcggtcaccaggggcatagcattgctcacccctgaatcaagtcatcgagatttctctgacctctgaagtgcactgtggttgcgtggctgggaatccacgcttgaccatgtactgcttgatagagtcgcggctggccgactcatgggttaaagtcagttgacaagacac

SEQ ID NO: 440 - DNA, 513 bp, Kobuvirus sp.
ccaccgttacttcactccactccctcgggactggtttggaggagcataacagggcttcccatccctgttcaccctcaataccacccaccctttccctcaaccatccctatccacaccccactgactgattcccttggattttgacctcagaacgcctacttgacctcccacttgcctttcccttctcggattgccggtggtgcctggcggaaaaagcacaagtgtgttgcaggctaccaaactcctacccgacaaaggtacgtgtccgcgtgctgagtaatgggataggagatgcctacaacaggctcgcccatgagtagagcatggactgcggtgcatgtgacttcggtcaccacgggcatagcattgctcacccgtgaatcaagtcattgagattcctctgacctctgaagtgcactgtggttgcgtggctgggaatccacgcttgaccatgtactgcttgatagagtcgcggctggccgactcatgggttaaagtcagttgataagacac

SEQ ID NO: 441 - DNA, 675 bp, Kobuvirus sp.
gggggtggggggggcctcggccccctcaccctcttttccggtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 442 - DNA, 675 bp, Kobuvirus sp.
gggctataaatatgggcattcctcttcccccttccccttttgaagatgagtgcgcatattcttgactccgcctggattggccgcccaaggcgtgaacaagcagctaggccaccatgacactgcggtggtgtccgaacccgcgggtgccttcacgggcacctgtggtatgtaggactcccaccgtggtcttccctttccccctcaatctttccccctggttcgactaacgggaccagtgctggaacctgtccggtgaacggtatagcaggcccccccggcagaaacacccggtgcttaccccttaaggctagcccccttccatgaatttggttggggcaactagtgggtgtacagttggcgtgaaccctccggtctaggagtgctcttgcccaatcctctgtgtgtgccttgcagtagggactggcaatccttcgcgtaggtgatccgctgtgccatgccatcctggcgacaggaggcccagtgtgcgcaacctacgtcccttctgggtgctgcattgcattacctttggagtaagcttggtgtgccgaaaccccagggtttacgtaccactcgtggtgtgaggaatgtgccgcaggtaccccatccttgaggtgggatctgagcggtagctaattgtctagcaccactttcttccttttttctttgctggtcacg

SEQ ID NO: 443 - DNA, 604 bp, Aalivirus sp.
ttgaaagggggtgctcagggtagctccctgagctcttccctccaccctctttcaacgtctggcccacgatacgggccacctttcaatcttaactaactatccctttaatctatttggattttctggtttagaataatttggaacacataattggattatcttttaggattgtggataggatttgttcgggatatcactcccttcctgtgctaacacatattctaattccctcctttgtctattatctcttggaggtggtgctgaaatattgcaagccacttgagtgtatagatgaagtaggctcaagatgaatgttgtgttactcaaggcaagtgtagctatcactaagatattggtaacgtgaaacggattaccggtagtagcgtgatcttccgtcttagtgctctagtgactagaggacaacgacatggcatcacatatcttaaccctccagttttggcatccgggacagaatgggctggatatccgctttctttctggggtatgtgatgggtggtattggggtaaccaccttgaccatgacgctcgataagagtgaccgcctgatcattgaaacctctagtataaaattcaggctgaaatc

SEQ ID NO: 444 - DNA, 445 bp, Grusopivirus A
tgcctgagtaggattgtgaatttaggtatgagagggttagccaacccattctgaaccataatagatacgtcaatctgaatccatctaaatctatctcttaggcagtggtgctgaaatattgcaagctactagggatagacgtgatctgattcaagaacctatctaatgtggtgatgagaaggctaggtttatccatagtaatcccttgttctgaacaggcaatgcacatgctctagtaggatctcgggctctgcgattggctctaaaccgaccaatccaggtagaggcactaagtgtaggacttgccaaaatgtattacatgctggtaccgactcactagtctggaaactccacactgaaagtgactggggggggccccatcacatttgtgctactgcttgatagagttgcggctggtcaacttggattggtataaccagttgaa

SEQ ID NO: 445 - DNA, 537 bp, Grusopivirus B
gccatccgtaggttctggtaaggttccatcaactgttggggcgctagttgctatgaccgcattcacggacggatgatttatagtatcacccaatccgggcacaacttctttagccacttcttccacattactaagggctctcttgccgagtttcaacgtctagtccacgacacggaccttcctacttttctatcttcttattttctctactaaattggtatctggtactgaagatatgcggattgtgattttgtgcctgtctaaactaaccctattctagggttaggtgggtaccatatactaatggtgaacaggattacctatgtatccattagtccctatggatctggcgacccacaaactcatgttcatagagaggctaagctgagtgctcgccgaataagcattgcttcaggtgccgactattgtctggaaaccactcagtgatagctataggggggggccccgtagcatctgccttactgcctgatagggtggcggctggtccatgaacatgcagtaaccagttgacttgac

SEQ ID NO: 446 - DNA, 562 bp, Unknown
  Description of Unknown Yancheng osbecks grenadier anchovy picornavirus sequence
ccttagggtctggaatgcgtcctttctgggcacttccacaatcctaaggtaattttcaacgccagcgatggagcgatatccaaagaacctttatgttttagttcgtcttgtatgtttataaaatataaaattgggattagcaacccacaaaaacatttttgttattctcaccacatgagagggtggttaaacctcttcgtaacctatcttgcttgattggctacttgggctagatttaggacaacctctttagcaagcctaaattactcctcgtacttgcacctggtaacaggcgtacctggaggttacggtggcgctaacttggacttctcgttaattcgtgcaagtttaaatgatgcctattttgaatacaagaaagtatgatagtaacttagggcgtgaagttccgcttaacataaggcagtataagtactaagataaggtgtaagacctaccttaataactgttgtcttttctcatggtctttccgtgggagcccttgctaggggtaaagttaagtattctcaaataatttttcattcaaactctttctctctgtttt

SEQ ID NO: 447 - DNA, 675 bp, Gallivirus sp.
ccactcgcacttcctggatagtgcgttagatatgcccgacatatcgcttccaggaccaaagccccccttttctctttcccaaccagcttcgccactcaagctgtaattccatgtccggtctttccggccttagtatcatggaaatgtggtcgtgctcaaatgaaattgagttgacattgatcaatgaaagttgcactgaactttgctaaactggctagcgccacctggtgtgtgccgttggtctcctcacatggtaacatgtgccaacgggcccgaaaggctagtgggcaattaccgctccaagggaggggtacccaccccgacctgaacagcggtaatgaagctcacctcccaggctctgaccccgagaagtttagttatttagtaggtgtaattagtacttgtgattggtcaatttgatagtagtttgaaacgttatggatgaatgagtagaccccctgaaggtaccccattacatgggatctgatcagggccacattctgcgtgtctccccgcacttgtggttaaaaccatgaaagttcatcccaaacaatcttttcctcttctttttcttttagtggtgacaacctactggattggtgattaccaatctgtactagtgttgtattaagacttgttgtgtggagaaaatggactctttcaagaagatttttg

SEQ ID NO: 448 - DNA, 673 bp, Unknown
  Description of Unknown Falcovirus A1 sequence
taaaaggggacgcggtgtggcagctttggctgtcatgccgtgttctccttttaccccaaggactagccttgggggttttccaaattcctttccctgtaggctttacttctctttatctatcttttctgtaactaagttttgcctattctaaaaatattttagaatgtgtttggatgtaactaagtttgtgcctgccctaaaaatattttagggcttgtttggataacctcgtcccttgtgttcagtgccgcacaatttgctaggcactgttcacttcctttgtttgtccattatgtatgctaaggtatgaattccatcatatgcttagcctctacatgcataatcttattccctccctggtgcaaactacgcccccaacatatgtgaatcttttaagcatattcctgaccccacacatatatatgtgttctcgtgaattcccccaccgtgaggtggtcacttggacgtggtgtgtgtcacacagcatatatatgatgcaggatgttgtttttaagataagcatatgtccttagtgctttgcatcatttcctccacaccccgtgaatgcggctaatcttaaccctgttgggtccgtgggtaaaccaacccattaaccacaggacggaaccgactactttcgggagtgtgtgtttctttttcttcttttgtcact

SEQ ID NO: 449 - DNA, 665 bp, Tremovirus B
ttcaaatggcccctgggttgatacccagtggtcatttggacactttggtaaggaggtgtaattatccttcccatgtggaacctagtgcttaggtttactttatatgttctttgtttgtcctttgtactttctatcgggcaatcttgttgttcaatacaatatgtatttgaactgcctaagataaattcagttttcaaccaacccctctcttggggttgtgtctttctttctttcttatatcctcttaagctgacttacttgctaatccgactcctcgtcaacgggagggtaaagcagtatcactagggtattgtgatgtaggagaaaaagtaagtagagatagtgcatgtaacgaaagtgacttggtactttaaactctcttaatcccaaagtgtggtattggtcatgttggagtaggctacgggtgaaactccttcacatttagtaatgtgttcacacgctaacgctacggtagatgacagactaggtcttattctcaacgtagggggacgggtgtatgttcatgattagccacatattaaggttttgaggggctgagtcatataagtatgtgcattaatttctggtactggtccctggggactggcccttttctaggttgattttagtttccccaatttttaaaaactaatgagatttacgac

SEQ ID NO: 450 - DNA, 675 bp, Hepatovirus A
tctttggtctggggaactaaaataccagacccgcgtttgcctagcgatataggctttaattgttgtttgtcattgtgcgtttgatatgtgttttaatgtaaataataattctagcaggttctagttcttgatcatgtcctctttaaggcactcatttcaacttgctatctttcttttcttccttggttctccctacaccaaatgcactggccgctgcgcccggcggggtcaaccacatgattagcatgtggctgtaggtgttgaaggctgggacatgaacatcaatggaatagtgcgcatgcttactggggtccattgaagtagtgggatctttctattggggtaggctacgggtgaaaccccttaggttaatactcatattgagagataccttggataggttaactgtgctggatatggttgagtttaacgacaaaaagccatcaacagctgtggacagaacctcatccttagattgctcactatggatatgtgctctgggcgtgtttcttgcatgatggccattggtcaattcatgcctgggccaatgtaggattagccttaaattactttttaaaagtagcctcatttagctggactaatggtggggcgtatgatcctgcatttggcctctggggtaatcaggggcatttaggtttccacataatagcaaat

SEQ ID NO: 451 - DNA, 675 bp, Hepatovirus sp.
gcaaggggtggttttaaccttgcacgcgtttaccgtgcgttaacggttttccatgtttgtatgtcttgtttgtattatgtgttttgtaaatattaattcctgcaggttcagggttctttaatcatgttgggctgtacccacactcaacttttggccataagtgagtttcttaacgaaccttttaacacaggatgttattagggcccaatattttccctgaggccttctttggcctctattttttccccttttctatctccttgtattccgggctcacgtgatgccaatggactgacccatgcgcccgtgggggttaactactggagtagccagtagctgtaggtgctaaaagtcacgtacgtgtaagactggacgagacctctcagctataactgaaagtagtaagtatgtctgaacttcttgaaggggtaggctacgggtgaaaccccttaggttaatactcatattgagagatacctctgataggtgaaggtttccggtagaggtgagtttaacgacaaagcctctcaacggatgtgggcccacctcatcagcaagatgctttcatacccaataccgtaggggctgggttgttgagttcagtcccaagcgtccctcccgcaaggttgtaggggtactcaggggcatttaggtttccacaattaaacaaataca

SEQ ID NO: 452 - DNA, 675 bp, Hepatovirus D
ctttggatgcccatagtgcgggggtataaataccgcactccctttagctgttccgagggtatcggaacctatatgtttgttttctgtctgtctgtcagctttatgtgtgctcgtcccctttagggcactcatttcagcttgctttcattcttttcttccccggttctcaccttaccggaggcactggccgttgcgcccggcggggtcaacctagtgattagcactaggctgtaggtgtctaaagtggtgacattaagacttggtaactgatttcagcactgttaactgatgttggggatgacttgattgatcttctggaaggggtaggctacgggtgaaaccccttatcttaataccactatgtagagatagattcagtaggttaagggcagtggataaggttgagttcattttggacaataaaccttcaacactggtggacccaatctcactgaccagatgctttcttgactgatccttcagaggggtgattcttctgaataggttgccttgacactgatgcctgagacccattgggtcgggccttaaatcatggaactccactggactttcatggcctagcttctgccttagacagactctggggccccacgaccctctgggcccttcggggtactcaggggcatttaggtttttccacaattaaaagagtta

SEQ ID NO: 453 - DNA, 560 bp, Hepatovirus sp.
gtcatgtttctctttaagaacactcaattttggccataagtgagactcttgtcgaacctttcatgtcaggaccatgttagggccattatccttttccctggggcattcttcttgcccctgtttcatctttctatcatctttcttccgggctctcacaatgccaatggagcgaccgatgcgcacgtcggggttaacccatggattagccatgggctgtagctgctaaaagttgtgactcctgaagcatactatcaatggtagtagatgtaactgaaacactgaagcttctctgatcttgaaagaagggtaggctacgggtgaaacccttcaggttaatactcatattgagagatacctttggtaggttaacgttggcggataatgttgagtttaacgacaataaacattcaacgcctgtgggcgaacctcaccaatttcatgctttgaagtgaatgtgcgtagggtctctatcggagatgctatgtggatggtgccctccctggaaacaggttgtaggggtactcaggtgcacttaggtttccacattttaaagatttttc

SEQ ID NO: 454 - DNA, 675 bp, Hepatovirus I
ggctgcctgtgtctcaggggtaagtactggggccgcgttgaccgtgcggtacggttatgcttttagattaggatgtccgtctgtccggcactctcttttgcttaaaatggccttaaatccatgggaggcgtaaccatgggccctttgttacctagacatgattgcattgggggccgtccttggggcttaggccccagccatttctcttgactcgtctaagagtttacttcatccttttctttactttattttccaggctctcagcatgccgacggctctgaccactgcgcccggtggggttaactgcatgattagcatgcagctgtaggagttaaaagtgctgacaggccaattctgacgtaagtccactctatattaacttgatcaagtaaggttgattgatctttgtgagagggtaggctacgggtgaaaccctctaggttaatactcatattgagagatacctccagaaggtgaaggttggcggatattggtgagttcttttaggacaaaaacctttcaacgcctgtgggcccacctcactggcacaatgctttcatccccaattgtgatgggtagtttggactgaaatcaggagtaacctgccctacgagtttaggggtagttcaggggtatttaggcttccacatttgatagagtttatgagagtgagcc

SEQ ID NO: 455 - DNA, 527 bp, Hepatovirus C
ttcaaaagccccagcggggtttcattaccccgctgtggcttttggacttccctaggatggggaagtaaattaccatcctcgcgtttgccgtgcgttaacggctacttttcttctagctgtagaagtaaaattcagcatgttttatgtttgtttgtcttgtttgttatatacatttttacactcctacaaatgcacatgaagaacagtttgtagagattaacaaacgcttagctgaacctaggtggtgaatctagtagtaagataagtagaggaagctataccttaagttggttgggccctcgtgtttgctctataaacaaaaccaagtgagtagagtggatgaacagtactaaatccctgagtacagggaacctcacaggtgtgatacacttatgtctatgtgacctggttggaggttgggcgtgccctatgatactggagtgggagatcttttggggaacccacgttttcacactgcctgatagggtcttgccgagagactcacttgtttcggctgtacttgtaac

SEQ ID NO: 456 - DNA, 665 bp, Fipivirus A
tgcgggtaaactcccgcatgtgtgaatgaggcgatgtcccaggaactaactgccgatcctggttttaactacgatccgtatttgttactaatgcgatatccccccattgtttgcctccatgttgttttcaacgcttttggccttgagtgttatcaagtgttttagcgacatagtgggaagctacggctgcgtccccatttttgagtggcgacccagttttagtggccactctgtccctgaactgcgctataatgtgaatttatgttcacaaaaacggactgatgtaactgttaatgactaaggaatagtacctcactgaagtatcaagaccccgttcgagcggtgtacatatatggatggaaaccttgtctgagtcatctcgaatactaatcaatgagggatgtcgagtaagcatatcatgaaccacatagaatagtggggtttcggggttagaggctctctgcagcaatgtatctctaacaccatggccgaaatgagagatagagaccacgatgtttgtgtgtaagtaatgatgtgtggaaagaaaattctgaatgttggtatgatatcagtctaaggggagtggctcacctaagagctacccaaacatttcacagcagacaacataacgtactgagagtagttggaaggttccagaaatcagt

SEQ ID NO: 457 - DNA, 467 bp, Fipivirus C
cgcggttaaacccgcgcaaccttctttcagccgcgtctgagtagcgcggttagtcctgatacacagtttcctgttgggtactgtgtcttcgggtgaatgctcttgtgtgaatgttttaggctgtttaagggaagcgtttccccgtgcgctgtgagggtttctcacgctctttcggggtgcagtctcttctgttgttcattaagatgtatggatgcactgttgtgaaggatttgtgaactggggatcgacaccccgtgaggggtgccccagtgtccataggagtttgctggagaggtgtgttgctgtagtgactatccgtgacctggcattctaaggtgttgaccccaacctgtgagggtctggatcgcagtgttgaagtgctttggagggttcaatggggtttctgtagtggatattatgtgcttgacgactactggtacgagtgtattgggggtctacatgtgtga

SEQ ID NO: 458 - DNA, 657 bp, Fipivirus E
ctcttccgatcttgggggttcgcccccatgtctcatttcaactagccgtgtgtctagttaacgcaccgcctcaccctggtcgttatcgggtcggttcttgcgaccgttagatcgtgagcgtttctgaggatcagttcgtataagttctccggtgtggcgaccgtaaaatcgtcacgtcccatgcaatagatgacgttaaactcgtttgccagttacataaaggaatgttgttacttttaaattgtctgttacatttaacatcttgccagtatgatgctactgtacactacgggtgtaggaaccttgtagtgtgacgtatcactcatatgtggatgggtgctccagacctttatggaagctctcagttagtagtgatccttgacttcattgagccctggtaacagtggaagtcaagatgtatatgttgctcaacacacttcggtgctacgaagctgtttgtggaagtactggcgaggttcattctgaatcatatgtttgtcacatagtcagggagtgccgtcgcttacgacggaccctttttctttataattacaaatctgtgtctcaagtgttgttggctggttttcttcttctgttttcattgttcatatatatacgtcagagtgaaagactcggtatatacaaaactgatccaga

SEQ ID NO: 459 - DNA, 506 bp, Aquamavirus sp.
ttcaaaggtggcgggagagttggcctcacgctgtttagcgtgagagctggctctcctgccccttcccctgagccggggatcttggctcattcccctcttttctatcctccctcattggactttacggatgacccggcataaacttgacaaccgatgttggatttcccttgtggctgtgatggaggacataccctcgggtgtagttgtgtgcgtgtcgctctgcgactcgagcttcaaagtggtgctgaaatattgcaagcgtcgttgctcgattaacggagtggtacaatcctatgaacccaagtgcattcatgcgaaagccccggaggggtgagtagcatggactcgaatcagaagagctggagctcgcttggtacggcacgtagcattgctttgcctaaagaccaagggggtatggctataggtgggggcctatagcttgtccagtgctggttgacagactcgtgctacgcgtctggttcgagtataagtagctgcaactcact

SEQ ID NO: 460 - DNA, 548 bp, Avisivirus A
ttcactcgctttccccccctctctataggggcggtcttttaattcttattaatttcctactttactatcaaatttcttctaagtagggactgaggtcacttagccctccctctcctgggctttccagggttatagaggttctaaagctaagccatgtgtcttgagctacacttagtacaaaggtttagtaatgattgtacatgccagtaaccttctagtgcccatggattaaagagtggtaacactctccatggggcccgaaaggctagtgggcatagttggcatcaaggaaggggtccccaccccaacctgaattgctggctagaagctcaccttagaagaagtgctgggtgacaacgtgtccaatcgtgaacgactgatggaaacgtgtggagatggatatgtgggggttcactgagtagatgccctgaaggagaaatctgatcaggggcccgtgactatacgctaggtaaaccgggtataaaaaccatgaaaggtggcccaaaatctcttccttttattttatttctatgttggtgacagtcaag

SEQ ID NO: 461 - DNA, 492 bp, Avisivirus B
caccccctactgccctaacccccaaagttagttatagggtggctccctacccttactccacggggtaagccctaacccggttgaatctcaagatcagccttagcgaggactattagtaccgctcaaaccctttgcctgtagtgcccaggggtcacagaggggtgaccctctccctggggcccaaaaggctaggtggcaagacagggtccaagtgaggggctactctaagtagccccaagctgaacatcctgtctgaagccacccttgcagggccaggtttgattggggaaactagacaccagctttgtcctgggattggggggatatcgagttagtccaggaggtgcgagtagatgcccccgaaggtaccccaggcacatctgggatctgatcgggggcccgtgactatacaataggtaaaccgggttaaaaaacatgaaagcgcctctctctttcctacttcttttattgactggtgacaaaaatagcagt

SEQ ID NO: 462 - DNA, 517 bp, Crohivirus A
gttgaagtccatttcttgcttgcccccgatgaatcctgttaaggcctcacggccctaagggtgaaactcggttatcccctcctgtacttcgagaagattagtacaacactatgaaatctacatcttgtgatccgggataaccccaatcccagaaacctgtgatgggcgtcaccacccctcttatggtaacataagggtgtcgccgcgttggcacaggaccctttgggctggatgtttttagtaatggtgtcgaaggtcctattgagctacaggagtttcctccgccctggtgaatgcggctaatcttatccctgagcctaaggttgcgatccagcaacttgatggtcgtaatgcgtaagttgggggcggaaccgactactttccagaaggcgtgtttctttgttttgtctgttactatggtgcatgatatagatattgaatatttgatctttttgagctgtttcttatcttattgctacatcctttcaggtgttggatttacattttggttaataag

SEQ ID NO: 463 - DNA, 393 bp, Kunsagivirus B
gattttctggttatcccttttggacttggtaggggcccacgtgcccacccacctctgtgtgtgttgatttctaatcgatgcctggcagtggcggccacctctccttactggtaaacctccggtgagtgaagttgtcaagctacaggtaccgtgcaggatgaaatgcgcacatgtgaacaaactaggagtcatacaccgggtcaaactctggaaacggagtccgggactctgaccttggttgggtgagctcgaggcatcacattgatggacgcgattcgctatccttccctagtaggaccttgtggtgtacccctggttgggaatccagggctggtcgggtgcagggtgacagcctgttctccacctcaaccattgtaggagaaatcaacccct

SEQ ID NO: 464 - DNA, 675 bp, Limnipivirus A
ttctttggatatccatttaacgtgtaccctatacgataattggggtggattctggatgcctagttccagtgattggttaagaactcgtttactacgtatagtatgattagcaaagtgctcgattgatcacgtaatgatctatgtggttaaaaacccagtagtatggtatatactcagtagtgtacactgtgagtacaactcttggcgtagagagaacaattcacccgaatccgtggcgtatccatggaaataagtttacctaattgtatgttacaaggcatatgagacatttatgagatatggtttattttgactaaacgagtgtagaggtggtggagtctatccaacttcaagccatgcaattgttgtgttgattgatatcattgaccatttttgtggattgtgtacacatacaatttgaaaattaaccccctcaagaataagacatgggaccattcgtggtagataccgtgctcggatgcttgagattagatgggttagactagttttggaatgagattgccgagaaagtcccgctagacatgttttacaagtcgtggtattccgctagactttttcgcagacacatggaagggtccatgtgttgtgcaattgcagggtgacagcccaactgcagagttttccttactagaataaaaatctgttgtcaatttt

SEQ ID NO: 465 - DNA, 501 bp, Limnipivirus C
gtttctgagcactggtaagagcttagacaaacgtttttaaaatttattttctctgcaacttttgtttgtgtttatttttatttgttaattttgcgcctaagcatttgttgcgaagtatttgattcattagtaatattacttattgtttatttagatggtattcaaagtggtgggagtatcgaacccaagcgtcgtatgctatctccttgaacaatttttaatcattgcgaagtgatcattgaaaaggataggtgtttaagaactcaaagagtgttaataatgttgggtgacaggtgtccccatagaatttattaacatgatttggactggttatctagtaagaagaaccatcgaacgcacgagcgagcattgcttgcggggcagttaccctgcgtcgatgtaagtgtgtaccggggggtgcacatgttgattctttatggcctgatagggtgcgtcattcgcgcctagataattagtataatgcgaatggaataaatttac

SEQ ID NO: 466 - DNA, 675 bp, Orivirus sp.
ggtcccaggccaatattcttcgtaaggcttggttccaattttccaccactcgtgtttgggttctggcctatggtacccagaggggcggtttgggggaattaactccccctcccctgtggtcctataccaccccacacctctgtgggctttctttactatcttcttgttttccgacttttaaacactaggcaggcgcgcctagtcatacaccgcccggctggtctttccagctcttgtgggcggtgcgcgctggtccatcgtgcccagcgacatagcaccttgtggacacctccgaacgccctcccctgtatggggtggtgcccaggggtttcagtgtggtgacacactccctggggcccgaaaggctagtgtgcaacaggtgaggtacagccagctgcccccgtggctggagggaccaagcttgtgaagcacacctcaccttcttgggggtgggctagtaagtggtgaaagcatagtgtccgtgtcgctggccaacactttgggtcaagtccagccactcagtgagtagatgcccaggaggtacccctagtggatctgacttggggcctgttacttaatgcaggttaaaaactatgaaagctgagtagtgtagcccggctggtggcttctcttccttattcattctattttatggtgacaaacgcaactgaagcc

SEQ ID NO: 467 - DNA, 675 bp, Hepatovirus A
cttgatacctcaccgccgtttgcctaggctataggctaaatttccctttccctgtcctttccctatttccttttgttttgtttgtaaatattaattcctgcaggttcagggttctttaatctgtttctctataagaacactcaattttcacgctttctgtctcctttcttccagggctctccccttgccctaggctctggccgttgcgcccggcggggtcaactccatgattagcatggagctgtaggagtctaaattggggacgcagatgtttgggacgtcgccttgcagtgttaacttggctttcatgaacctctttgatcttccacaaggggtaggctacgggtgaaacctcttaggctaatacttctatgaagagatgccttggatagggtaacagcggcggatattggtgagttgttaagacaaaaaccattcaacgccgaaggactggctctcatccagtggatgcattgagggaattgattgtcagggctgtctctaggtttaatctcagacctctctgtgcttagggcaaacactatttggccttaaatgggatcctgtgagagggggtccctccattgacagctggactgttctttggggccttatgtggtgtttgcctctgaggtactcaggggcatttaggtttttcctcattcttaaacaata

SEQ ID NO: 468 - DNA, 675 bp, Hepatovirus A
cgccgtttgcctaggctataggctaaattttccctttcccttttccctttcctattccctttgttttgcttgtaaatattgatttgtaaatattgattcctgcaggttcagggttcttaaatctgtttctctataagaacactcatttcacgctttctgtcttctttcttccagggctctccccttgccctaggctctggccgttgcgcccggcggggtcaactccatgattagcatggagctgtaggagtctaaattggggacacagatgtttggaacgtcaccttgcagtgttaacttggctttcatgaatctctttgatcttccacaaggggtaggctacgggtgaaacctcttaggctaatacttctatgaagagatgccttggatagggtaacagcggcggatattggtgagttgttaagacaaaaaccattcaacgccggaggactgactctcatccagtggatgcattgagtggattgactgtcggggctgtctttaggcttaattccagacctctctgtgcttggggcaaacatcatttggccttaaatgggattctgtgagaggggatccctccattgccagctggactgttctttggggccttatgtggtgtttgccgctgaggtactcaggggcatttaggtttttcctcattcttaaataata

SEQ ID NO: 469 - DNA, 519 bp, Parechovirus F
ggtcggggagatgtgttcatgatcggttaacaccatcatggatcatctctccccgacctctttttgacccagctatgggttaaatagtacttttcttttctcttttgctttcttttgtgttttgtttgttttgcaacatataacaagcattttatcagtattagtgtctgcaactgtataacaagcaaggtggagcaatcatgcgagtatatctcaattgaattgtgacacacaagtgtgcactatgtggaataaatgccattttggccaaacctggttagccagaccagtagtaggacaatttggcacccttagtgggcgcgacctagatgctagggatgagcaaacctatttcccctgagtacaggggctctccttcacctctacattttggacctctttttgagtatcctcgatagaaggtgaagtgacggtgtaccggatggttaattgatctcattgctgggtgacagcccgctaggaccaggcagcatctttgtatggacctgtacatgtaac

SEQ ID NO: 470 - DNA, 343 bp, Parechovirus D
acatggggcaggtgtgctgtgccaagagcaacactacggtggccgagccgatggttcgtcaccacgtagtaggactccgtagtgcttggttacggcggacgtaagtcagttgagtgatgtctaagtggcaaaccatgagtacatggtaaccttgtgtggactcgcgggacggaatttcctatcccattgactccttgtagcaaggtgggtatacccaaccacaatggcagcaccctgggtgggaacccaggggcctggattagtatccagtcacacagcctgatagggtggcggctcagccactgaccagcgtctctaaataattgtgagctgttcatgcacc

SEQ ID NO: 471 - DNA, 675 bp, Parechovirus C
cggtcatccccctttccccacagccggtgtgggttctaatcggctcctactaaacacctaagcatcactgcgcctctatctctcctatccacaggtctaagacgcttggaataagacatgtgggtgcaataggaagattagctagtccaatctctccttccagctacgcttctcccttcgatgagcgtagggggggcccccacctccctcatctctggatagggctcttgctacggggctttcccgtctggaccagcaggcccactggtgcgcttccattcaagtttagtgtgcattactgtctgaaatattgctttgctaggatctagtgtagcgacctgcatattgccagcggacttccccacatggtaacatgtgcctctgggcccaaaaggcatgtctttgaccgtatgcagtacaaccccagtataggtcctttctatggcagtatggatctcagtgatgagtctatacagaatatggaagtggttcggatatgtcagcccgaaggatgcccagaaggtacccgcagataaccttaagagactgtggatctgatctggggcccaccaccttcgggtgggtagaagctaaccatgccttgggttaaaaaacgtctaagggctgaccagacccgggggatccgggttttccctatcttgacctactctaatc

SEQ ID NO: 472 - DNA, 675 bp, Unknown
  Description of Unknown Ljungan Virus 87-012 sequence
ctcattgcccacacctggttggttcccaggttcatacaataaccatcaataaacttttaacatctaagatagtattatcccatactagactggacgaagccgcttggaataagtctagtcttatcttgtatgtgtcctgcactgaacttgtttctgtctctggagtgctctacacttcagtaggggctgtacccgggcggtcccactcttcacaggaatctgcacaggtggctttcacctctggacagtgcattccacacccgctccacggtagaagatgatgtgtgtctttgcttgtgaaaagcttgtgaaaatcgtgtgtaggcgtagcggctacttgagtgccagcggattacccctagtggtaacactagcctctgggcccaaaaggcatgtcatttgaccactcaggtacacaaccccagtgatgcacacgcttagtaatggcttagtaacaaacattgattgatcatttgaaagctgttaggaggtttaggtatgacgggctgaaggatgccctgaaggtacccataggtaaccttaagcgactatggatctgatcaggggcccaccatgtaacacatgggtagaagtcttcggaccttgggttaaaaaacgtctaggcccgccccccacagggatgtggggtttcccttataaccccaatattgtata

SEQ ID NO: 473 - DNA, 675 bp, Parechovirus sp.
gccgtcgggccttacaccccgacttgctgagtttctctaggagagtccctttcccagccagaggtggctggtcaaacaataccaaacgtaactaaacatctaagataacatagccctatgcctggtctccaccagttgaaggcatcttgcaataaaatgggtggattaagacgcttaaagcatggagtcaattatcttttctaactagtgatcttcactgggtggcagatggcgtgccataactctattagtgggataccacgctcgtggatcttatgcccacacagccatcctctagtaagtttgcaaggtgtctgatgaggcgtgggaacttattggaaataattacttgctgcgaagcatcctactgccagcggatcaacacctggtaacaggtgcccctggggccaaaagccacggtttaacagaccctttaggattggttaaaacctgagtaattatggaagatacttagtacctaccaacttggtaacagtgcaaacactagttgtaaggcccacgaaggatgcccagaaggtacccgcaggtaacaagagacactgtggatctgatctggggccacctacctctatcctggtgaggtggttaaaaaacgtctagtgggccaaacccaggggggatccctggtttccttattttagtgtaaatgtcatt

SEQ ID NO: 474 - DNA, 631 bp, Parechovirus sp.
agagtccttttcccagccagaggtggctggttaaataatacctactgtaacaaaacatctaagatgtaacaaccacacacctggtctccactggccgaaggcaactagcaataaggcaggtgggttcagacgcttaaagtgtgttgtacatattcttttctaacctgtgttttacacagggtggcagatggcgtgccataactctaacagtgagataccacgcttgtggaccttatgctcacacagccatcctctagtaagtttgtaagatgtctgatgacgtgtgggaacctgttggagataacagtttgctgcaaagcatcccactgccagcggatctacatctggtaacagatgcctctggggccaaaagccaaggtttaacagaccctttgggattggttcaaacctgaactgttatggaagacatttagtacctgctgatttggtagtaatgcaaacactagttgtaaggcccacgaaggatgcccagaaggtacccgtaggtaacaagtggcactatggatctgatctggggccagctacctctatcttggtgagttggttaaaaaacgtctagtgggccaaacccaggggggatcctggtttctttttaatttaagtaatcact

SEQ ID NO: 475 - DNA, 675 bp, Parechovirus sp.
gggccttataccccgacttgctgagtttctctaggagagtccctttcccagccctgaggcggctggataataaaggcctcacatgtaacaaacatctaagacaaaataatttgccttgcacctggtccccactagttgaaggcatctagcaataagatgagtggaacaaggacgcttaaagtgcaatgatagttatcttttctaacccactatttatagtggggtggtggatggcgcaccataattctaatagtgagataccacgcttgtggaccttatgctcacacagccatcctctagtaagtttgtgagacgtctggtgacgtgtgggaacttactggaaacaatgctttgccgtaaggctttcattagccagcggaccaccacctggtaacaggtgcctctggggccaaaagccaaggtttaatagaccctaatggaatggttcaaacctggagcattgtggaaagtacttagtacctgctgatctggtagtaatgcaaacactagttgtacggcccacgaaggatgcccagaaggtacccgtaggtaacaagtgacactatggatctgatctggggccaactacctctatcttggtgagttggttaaaaaacgtctagtgggccaaacccaggggggatccctggtttccttttattttactttgtcaat

SEQ ID NO: 476 - DNA, 433 bp, Parechovirus sp.
ctctattagtgagataccacgcttgtggaccttatgctcacacagccatcctctagtaagtttgtaagacgtctggtgacgtgtgggaacttgtgggaatcaatattttgctttaaagcatccattagccagcggataaaacacctggtaacaggtgcctctggggccaaaagccaaggtttaacagaccctagtggattggtttcaaaacctgaaatattgtggaacacactcagtacctactgatctggtagtaatgcaagcactagttgtaaggcccacgaaggatgcccagaaggtacctgtagggaacaagagacactatagatctgatctggggctggctacctctattttggtgagtcagttaaaaaacgtctagtgggccaaacccaggggggaccctggtttccatttattttacaaaggcact

SEQ ID NO: 477 - DNA, 488 bp, Potamipivirus A
cacatggaaagcttttcgcttccatgtttacgcacacactctctttgacaccctgttgtatggtgttaaactacaacatttgtctgtctataatcgtttattttgtttaccctatatgtacccaagtatttgattgcttgactcacataagcatcggtaacccatactgttttatgagctactacctctgctgtctacatacattttatatgaatggtttgagctctgcctcaggatcaaacatggtaacatgttcctttggtcagttagaatcttattgtataatctaaggtgtctattagtacgtagaaagttgtaacacatatggggcctgatagccgctatctctgatggatgtaaggtaaccttctttaggtctgatacattctgcacaggatccaattttcggtgccctgtacgagtgcactcttatgcacgaggacgagatatgctacaacccactgcaaatttaaacccaaactttaaca

SEQ ID NO: 478 - DNA, 675 bp, Potamipivirus B
tttcaacgtcgtggctgacgttaaaaagccacaattccacttaccttttaccttttatgtttaatgtttgttagttttgtgatctttaacaaatagatctaaataatttgttggtaaccaatctcggatgtttcggctgcattgtagtttatttatttcattttagttgtaggtggccactacgtcctggaatcatacatggtaacatgtacctcggcggttatccactattacgctaatctaagaatatttaaatgaaaatgtaagtgttacggctgactttgggcctgatagttaaatgctcgcactgacagatagtaccctcctttaggatcgattctgttacatgggatccattttggtgccccactgattcaacctctttgttgaaaaagagttagcatactacaaattttccaaacaaaaaccctttttaatgactacaacttatgattttatgaattttactgctcttgaaaaagatattttgacattgatcgctgtactgtttcagacattcattgcatccatttttgttggctactcctcacaaactcaaaacttttccacacgagaaaccttgtttattgaattttgcctttatattttggaacttgttgttggatttattgtttgcttaattattgacctcacacctgttttaaacactacaat

SEQ ID NO: 479 - DNA, 546 bp, Unknown
  Description of Unknown Beihai Conger Picornavirus sequence
gggacaaccccacagctggtacaaccattgtgggttggtctccaccctttttcaaccgtggcaacttcggttaaagttgcaaatcccccctctccctattccacctcccttactttcactccccatatatggtcccagattttattctacctctttatatttttatttagtacagtggtggtgaattactcccagcataaactttgctggatcagtgttcatcaagcatactaattactaatgtactgagctatactattatctggcatctcacctggataaccggtgtgaccatatttcctaggttgcctccctatgtattttgtagcacctgtgcatctgcacgttggggcgacaaattgtaggtttcctggcacgggtaagaattgtggaaagctagtatgcagttaatgcaagggcgcgtttttcgctaccccgacactgctaaagtttttgggaggggtcccttaaacatttctagtattgagtgatagctttgcggcaggtcaccacaaccttactataaataaacctgttgaatctcac

SEQ ID NO: 480 - DNA, 457 bp, Sapelovirus sp.
tacgcatgtattccacactcatttcccccctccacccttaaggtggttgtatccccataccttaccctcccttccacaatggacggacaaatggatttgacctcacggcaaacacatatggtatgatttcggatacaccttaacggcagtagcgtggcgagctatggaaaaatcgcaattgtcgatagccatgttagtgacgcgcttcggcgtgctcctttggtgattcggcgactggttacaggagagtaggcagtgagctatgggcaaacctctacagtattacttagagggaatgtgcaattgagacttgacgagcgtctcctcggagatgtggcgcatgctcttggcattaccatagtgagcttccaggttgggaaacctggactgggcctatactacctgatagggtcgcggctggccgcctgtaactagtatagtcagttgaaaccccccc

SEQ ID NO: 481 - DNA, 489 bp, Sapelovirus sp.
ttgaaatgggtgtggggtacatgcgtattacggtacgcatatattccacactcatttccccccctccacccttaaggtggttgtatccccataccttaccctcccttctaaaacagatggacaaatggatttgaacttatggcaagtgaatatggtatgactttggatacactttaacggcagtagcgtggcgagctatggaaaaatcgcaattgtcgatagccatgttagtgacgcgcttcggcgtgctcctttggtgattcggcgactggttacaggagagtaggcagtgagctatgggcaaacctctacagtattacttagagggaatgtgcaattgagacttgacgagcgtctcttagagatgtggcgcatgctcttggcattaccatagtgagcttccaggttgggaaacctggactgggcctatactacctgatagggtcgcggctggccgcctgtaactagtatagtcagttgaaacccccc

SEQ ID NO: 482 - DNA, 675 bp, Sapelovirus sp.
ccaaggatctgttgcataggcgttgtatcccctaaccttttacctacccatcccaataggactggtatttcggttttgattgagtaatggatactgattctatacctgttacccattcaggggaaaaatggagtttctttcatggatctgacttgatatgaccaagagtcaacactttgcgtgttggccgtatggaatgctttaaggtttattctttggattatgacttcagggttggccgcccaggataaaaggcaattgtggtaagtgatgttagtcattggtggttgaaacctgcctaagacgtcctaggtctacgctgtgcgggccgaagtaagcttaggaataacagggagtatgccattttctgctttcacccaacacgaccgtacacgaaagagctagaggcactttggggcaaagggaaaagctttgcttagcccgaatgttcatttgagtccttgacgaatgcgtcccgtctgtcccgacggtgaggcgtatggcgcatgctcatggcattacccaatggtgtatctgtgaggggggggctcctcacacttagtctagtgctacctgacagggccgcggctggtcgtttgtgtatggtataaccagtagtaatcccccatggattgctttaacttcccctcctcccttaccaagacattctctaag

SEQ ID NO: 483 - DNA, 579 bp, Sapelovirus sp.
ttttaacttgttatgacattcaaggaaaaaatgtctttttcattatgggactgacctgtttatgaacatgagcagcggcactgctccacgggctatccgtgtaagaaatattgattattcttatggatcatgatttcagggttggccgcccagtctaaaaggcaattgtggtaagctatgtaagtagttggctgttgaaaggagccaagtacatcctaggtctacgctgtgcgggccgaagtaagacttggaacaactctgagtaggcagtttttctctttagcccaacacgaccgcatactgaagagctagaggcactttggggcaaaggtaaaagcattgcttagaccgaatgttcaatgagaccttgacgagtgctgtcacagtgtcccctgatggcagtatggcgcatgctcttggcattacccatatgtgtatctatagggggggggccccctatacttagtctagtgctacctgacagggccgcggctggtcgtcggtgtgtggtataaccagtagtaatcccccatggattgctttaactccccctcctccctcaacaaaactttctctaag

SEQ ID NO: 484 - DNA, 522 bp, Rabovirus C
ccgggtataacccggagttttggggcaggtccaagccccacataggaacatacgatccacggatcgtgtgttcttttatgctttctaaccttaccctttgtaaccattacgctttacgccgcatggtgtttggcggcaccatgacgtggacaagaggttacgccattacgatatgtaccctccccttttggggagagaccgaccaattatggtacagtatccaactgtattgtggtcaagtacttctgtttccccggtgatgcgggataggctgtacccacggccaaaacctgctgatccgttacccgactcacatctacgaggaggctagtaaaaggcatgaagttcaagagtatgatccaaccagatccccactggtaaactagtgatgagggttcccgttccgaacatggcaacatgtgggttccctgcgttggcactaggccccttccgaggggtgctctgaagatggattgttgatgaagaccaatttgtgcatgtgtttatcctccggccctctga

SEQ ID NO: 485 - DNA, 551 bp, Rabovirus A
ccgaccccactggtcgaaggccacttggcaataagactggtggaacaaggtcgcctgtagttgattggaaccttctttctaatgacttatgtcagcggtgctactcacaccgtaactctcctaccctatccccacgcttgtggaactaggaggggatgagtgattcaagtaagtactgtcagaatggtgaaaataatctgattctgaaacgctatggatccatcgaaagatggggctacacgcctgcggaacaacacatggtaacatgtgccccaggggccgaaagccacggtgataggatcacccgtgtagtttgagatcatatcaatgttcatagtctagtaagatgatttgaaatctaactggtctgatggctaactgcttgtcttattgcggcctaaggatgtcctgcaggtacctttagagaaccttaagagactattgatctgagcaggagccaaggtggtctttcccagccttggttaaaaagcgtctaagccgcggcagggggcgggaggccccctttcctcccaaactataatatagattgt

SEQ ID NO: 486 - DNA, 675 bp, Parabovirus C
gatgtatccccatcccccagtgtgtatgccatactgcatagctcgcctatgccctatggattcacaaccctttcatataccctccctacccaaccccgtaaccacatgctttactccgcttggggttttgcggccccatgttgtgacgaaatggctacgcaatcaatgcggctaatggggcctgccgcttttaagtggccccagttagaagtttatgcacacccgcccattaggaggccaccagccaggtggtcagagggcaagcacttctgtttccccggtgaagtttgataagctgtgcccacggctgaagcagacagatccgttacccgcctcactactacgagacggctagtagtgtgtaatatccgaatttcattgatccgggtgttccccccacccagaaacgtgtgatgaggagcggcacccctcctatggcaacatagggcctctcctgcgctggcacacgggctctatgagcatgaaatcaggagaaagtcacacgaagaccttattgtgctagtgttgattcctccgcccccctgaatgcggctaatcccaactccggagcgcccgctggcaaacccgccagaagagcgtcgtaatgcgtaagtctggagcggaaccgactactttgggtgtggcgtgtttcctttatttcctttgtatttgtat

SEQ ID NO: 487 - DNA, 675 bp, Parabovirus B
aacccataatccattgtccatcaatgttttatgggggggaccctttctcccctccccctccaaataccttttacccctctgtaaccaagagtgtgcaaaatctatttactagcccagaattgcggcttctggggaggtttattcctcatgcctaacaagatgttacgcaaactccgggctacggccctgggcttttgccctaaagatttagaagtttacactatcgtccaacaggaggacaacaaaccagttgttctaaggacaagcacttctgtttccccggtgagactggatagactgtacccacggttgaaactggttgatccgttacccgactcactacttcgagaagattagtaggaaactgtgaaactgattccattgatccggatactttccccgtatccagaaactactgatgagggttgacttcccgactacggcgacgtagtgtcatccctgcgctggcagtaggcctctttgaggatggaagatgtggatcggtaaccgaaggtcctattgagctagtgtttatacctccggcctcctgaatgcggctaatcctaacccatgatctagtgctcacaaaccagtgagtagctagtcgtaacgcgtaagtcgtgggcggaaccgactactttggagtgaccgtgtttcctattttacttttgtttg

SEQ ID NO: 488 - DNA, 675 bp, Parabovirus sp.
accgttacgcaccactcagttggtgtttggtggcaccaatgatggaacaaaaggctacaccacttgggctacggcccgcgccaccttgtggcgcaaagacattagaagaatagcataccgcccactagggccctgcagccagcagggtaacgggcaagcacttctgtctccccggtagaacggtataggctgtacccacggccgaaaactgaactatcgttacccgactccgtacttcgcaaagcttagtaggaaactggaaagttcgagttattgacccggagtgttccccccactccagaaacgcgtgatgagggttgccaccccgaccatggcgacatggtgggcatccctgcgctggcacgcggcctctaagaggataactcgctcctactggtaaccgaagagccccgtgagctacggtttattcctccgcctccctgaatgcggctaatcctaacccatgagcagttgccatagatccatatggtggactgtcgtaacgcgtaagttgtgggcggaaccgactactttgggatggcgtgtttccttgttttctccatttgttgttgtatggtgacaagttatagatctcgatctatagcgtttcttgagagatttccaaacatttattcaagtcgtacaattcttgtgtttaagcagtacagtgtaagg

SEQ ID NO: 489 - DNA, 316 bp, Felipivirus sp.
gatgtcggatgacggctggccaccggggaaaaacggcaaatgtgcaccacctctgcaacccacgccgaccacgtttaaccatggcgttagtaggagtggaccactgcagtgggctctggtgtgcgacagtcagtggtagagtagacagtcctgactgggcaatgggaccgcgttgcgtatccctaggtggcatcgagattcctctgctacccaccagcgtggactcctatggggggggccccataggctaggtctatactgcctgatagggtcgcggctggtcgaccactgactgtataaccagttgtaactcact

SEQ ID NO: 490 - DNA, 553 bp, Boosepivirus A
ttgaaagacctcggcatatatcgttgtcacaacggtatatgtcgagatctttctccccaccccctccaattcccttttccccctcttgcaacttagaagtttgtacacacagggcaataggatacgtgatccagccaggacacgtgagctcaagcacttctgtttccccgtccccttcacgtactacgggaatgttagtaatttgtgtgcactttagtaaggttgatccgggattaaccccaaatcccagaaactggtgatgagcgttaccacccccgccgggcgaccggaaggtttcgctgcgttggcaccagggcttcggcaccagaaaaaggtaaagcaaatgaaggcgctactgtgctacgagaagtttcctccaggcccctgaatgcggctaatcctaaccagtgatccaccggtgcaaaaccatgtactaggtggtcgtaacgcgcaagtcgctggcggaaccgactactttgggtgtcctgtgtttccatattttattttattcaattttatggtgacaagagtaaagagatacagatttgcagcc

SEQ ID NO: 491 - DNA, 512 bp, Boosepivirus B
ttttctcccctccccctccaactaccttttccccctcttgtaacgctagaagtttgtgcaaaccgcctgtagggtactgcaatccagcagtgcataggctaagcttttcttgttaccccaccccacattatactgaggaggattgtgaaattgtgttagtatgggttagtagcggtgacccgggtaaccccaacccagaaactcacggatgagatgaacaggaccccacatggtaacgtgtgtgttcgtctgccccgcaaggtgaggccgtgagagctttgcacgcgaaaaccttgaaaacccaaaagtaccttgagctcttcgctattttgtgtttcctccaggaccctgaatgcggctaaacctaacccgcgatccgcacgtagcaacccagctagagtgtggtcgtaatgcgcaagttgcgggcggtaccgactactttggtgttcctgtgtttcctttattttattttgaatttttatggtgacaacagctagaaaataagagtgaac

SEQ ID NO: 492 - DNA, 506 bp, Unknown
  Description of Unknown Phacovirus Pf-CHK1 sequence
gtgtgtcatttctcccctccccctcccaaaccttttccccctctaatcggattgattaacccggttaaagatgattaatggtttgtgagttgatatgatggcccggcattgaatccgggaattcttaagtaatggaattgcatccaatatgaaagtgagtgtggcaagctcacaagtagtacttggctctgcccattatttgaggaccaactcttcttgactacaatgtgtttaaagtaaactggaccacattgtgtatccagacaactccatttgataatgtacgctggaaacgttttcagtgcatagggtcctaaagtggtgctgaaatattgcaagctcaatgggatactgaacgctgaaaaccgccgctgttatcatatgggcccctagtgggtaaatgttggctttaggcatatactgcttgggaatgcagtactggttgtagacagggtgatagcctaccggctggcgtagttgagttggtatagccagttgattgccat

SEQ ID NO: 493 - DNA, 612 bp, Rhinovirus sp.
ttaaagctggatcatggttgttcccaccatgattacccacgcggtgcagtggtcttgtattacggtacatttccataccagttttatacaccccaccccgaaactcatagaagtttgtacacaatgaccaataggtggtggccatccaggtcgctaatggtcaagcacttctgtttccccggcacccttgtatacgcttcacccgaggcgaaaaatgaggttgtcgttatccgcaaagtgcctacgaaaagcctagtaacactttgaaaacccatggttggtcgctcagctgtttacccaacagtagacctggcagatgaggctagacattccccaccagcgatggtggtctagcctgcgtggctgcctgcacaccctgccgggtgtgaagccagaaagtggacaaggtgtgaagagcctattgtgctcactttgagtcctccggcccctgaatgtggctaaccctaaccccgtagctgttgcatgtaacccaacatgtatgcagtcgtaatgggcaactatgggatgggaccaactactttgggtgtccgtgtttcctgttttactttttcattgcttatggtgacaattgtatctgatacacttgttacc

SEQ ID NO: 494 - DNA, 627 bp, Rhinovirus sp.
ttaaaacagcggatgggtatcccaccatccgacccacagggtgtagtgctctggtattttgtacctttgcacgcctgtttccccattgtacccctccttaaatttcctccccaagtaacgttagaagtttaaggaaacaaatgtacaataggaagcatcacatccagtggtgttatgtacaagcacttctgtttccccggagcgaggtataagtggtacccaccgccgaaagcctttaaccgttatccgccaatcaactacgtaatggctagtattaccatgtttgtgacttggtgttcgatcaggtggttccccccactagtttggtcgatgaggctaggaactccccacgggtgaccgtgtcctagcctgcgtggcggccaacccagcttttgctgggacgcctttttacagacatggtgtgaagacctgcatgtgcttgattgtgagtcctccggcccctgaatgcggctaaccttaaccccggagccttgcaacataatccaatgttgttgaggtcgtaatgagtaattctgggatgggaccgactactttgggtgtccgtgtttccttttattctttatattgtcttatggtcacagcatatatagcatatatactgtgatc

SEQ ID NO: 495 - DNA, 615 bp, Rhinovirus sp.
ttaaaactgggtttgggttgttcccacccaaaccacccacgcggtgttgtacactgttattccggtaaccttgtacgccagttttatatcccttcccccccttgtaacttagaagacatgcgaatcgaccaatagcaggcaatcaaccagattgtcaccggtcaagcacttctgtttccccggctctcgttgatatgctccaacagggcaaaaacaattggagtcgttacccgcaagatgcctacgcaaaacctagtagcatcttcgaagatttttggttggtcgctcagttgctaccccagcaatagacctggcagatgaggctagaaataccccactggtgacagtgttctagcctgcgtggctgcctgcacacccacacgggtgtgaagccaaagattggacaaggtgtgaagagtcacgtgtgctcatcttgagtcctccggcccctgaatgcggctaaccttaaccccgtagccattgctcgcaatccagcgagtatatggtcgtaatgagtaattacgggatgggaccgactactttgggtgtccgtgtttcactttttacttatcaatttgcttatggtgacaatatatatagatatatattgacacc

SEQ ID NO: 496 - DNA, 660 bp, Enterovirus L
acatgggccagcccaccacacccactgggtgtagtagtctggttctatggaacctttctacgcctcttttgcttccctcccccatttctccttcgattgctccacctgtgatctttgcaacttagaagaaataatgaacccgcacaatagcgggcgctgagccacagcgtcaatgtgcaagcacttctgtttccccggaatgggcccataggctgtacccacggctgaaagggaccggcccgttacccgccttggtactgcgagaatgttagtaactccctcgatagctttaggcgttacgctcagccctttgagcccgaagggtagttcgggtcgatgaggctcgtcattccccactggcgacagtgtgacttgcctgcgttggcggcccggggtggggggcaacccccatccacgcctactgaaggacagggtgtgaaggcgctattgcgctactaaggagtcctccggcccctgaatgcggctaacccgaaccccgagcccacggtggtaaacccgccacaagtgggtcgtaatgagtaatttggggcagggaccgactactttgggtgtccgtgtttcctgtttttccatacgatggctgcttatggtgacaaccataagcaattggattggccatccggtgttcatattgcgaat

SEQ ID NO: 497 - DNA, 675 bp, Enterovirus K
tcagcctgacgcaagtgcctccattggagtctctccaagccctccggggcttggagggcgccgaccccctgcctagggggagcccacgacacggctggagtccattggcacaccgcagccacgattcaagccagaattgaaagcgggaagcacttctgtctccccggtgtggatcatacgctgtacccacggcgaaaagtgaagcatcgttacccgactcggtacttcgagaagcccagtacagttgtggatctctgcagggtatacgctcagcgtgacccctacgtagttccttgagatggctgagagaacaccccacgggcgaccgtgtctctcggcgcgtggctcaaggccgggccttcagtggctcggtgccttgcagagtgaagcctccgaacagcctattgagctaccgtttagcctccgccctcttgaatgcggctaatcctaaccatggagcgcccgcccacagtccagtgggtagagcgtcgtaacgcgcaagtccgtggcggaaccgactactttagagtggcgtgtttccaatttatcctttataaagttgcttatggtgacaccacaagagatccacgatttcttgtttcttatcactgagacacaagtcatattcatcaatctttattgcggaattaacttggtgcgtccaaacacatcagc

SEQ ID NO: 498 - DNA, 675 bp, Enterovirus sp.
caccctgagggcccacgtggcgtagtactctggtatcaaggtacctttgtacgcctattttatttcccttcccccacagtaacttagaagcttatctcatagttcaacagtagggtcactaaccaagtggctcagcgaacaagcacttctgtttccccggtcctagtacctgtgaagctgtacccacggcggaaggggaaaaagatcgttatccggccccctacttcggaaagcctagtaacaccattgaagcaatcgagtgttgcgctcagcacagtaacccctgtgtagctttggttgatgagtctgggcactccccactggcgacagcggcccaggctgcgttggcggccaaccgactcgggcaaccgggtcggacgctcgtttgtggacatggtgtgaagagcctactgagctagagggtagtcctccggcccctgaatgcggataatcctaaccccggagcacccacactcaatccagagtgcaggatgtcgtaacgcgtaagtctgggacggaaccgactactttgggtgtccgtgtttcctgttttacttactttggctgcttatggtgacaatctagtgttgttaccatatagctattggattggccatccggtgttttgaattgtgtgtttatactaattcttttacatatcacagacaaccaaat

SEQ ID NO: 499 - DNA, 675 bp, Enterovirus sp.
cggtacctttgtacgcctattttacccccttccccttgtaacttagaagcaaagcaaaccagttcaatagtaagcaacacaacccagtgttgtgacgaacaagtacttctgtttccccgggagggtctgacggtaagctgtacccacggctgaagtatgacctaccgttaaccggctacctacttcgagaagtctagtaataccattgaagttttgttggcgttacgctcaacacactaccccgtgtgtagttttggctgatgagtcacggcattccccacgggcgaccgtggccgtggctgcgttgcggccaaccaaggggcgcaagctccttggacgtcacttaacagacatggtgtgaagaacctattgagctaggtagtagtcctccggcccctgaatgcggctaatcctaactccggagcacatcagtgcaacccagcatttggtgtgttgtaatacgcaagtctggagcggaaccgactactttgggtgtccgtgtttcctgttttaccttatttggctgcttatggtgacaatttgatattgttaccatatagctgttggattggccatccggatttttgaaagagacccaaaactttcttctctacttcagattcaagtgcgaagttttccttttcatatattacttactaatttgaagtaccaaag

SEQ ID NO: 500 - DNA, 675 bp, Enterovirus I
ttagtactttctcacggggatagtggtatccctccctagtaatttagaagacttgaaaaaccgaccaataggcacctcgcatccagcggggtaaaggtcaagcacttctgtttccccgggtcgagtagcgatagactgtgcccacggtcgaaggtgaaacaacccgttatccgactttgtacttcgggaagcctagtaccaccaaagattatgcttggggtttcgctcagcacgaccctggtgtagatcaggccgatggatcaccgcattcctcacaggcgactgtggcggtggtcgcgtggcagcctgccgatggggcaacccatcggacgccaagcatatgacagggtgtgaagagcctactgagctacaaagtattcctccggcccctgaatgcggctaatcccaaccacggagcatttgctaccaaaccaggtagtggaatgtcgtaacgggtaactctgtggcggaaccgactactttgggtgtccgtgtttccttttaatttatcattctgtatatggtgacaactatagtgctatctcgatttgcattactattgttgagattaaaactttattacattgttgcattttaccctttgagtgagttttcacctgaacagattaatttactcatcctgtttatatattacaagcagaaatacttgcaaag

SEQ ID NO: 501 - DNA, 675 bp, Enterovirus sp.
gcaatgctgcaccagtgcactggtacgctagtaccttttcacggagtagatggtatcccttaccccggaacctagaagattgcacacaaaccgaccaataggcgcaccgcatccagccgtgcagcggtcaagcacttctgtctccccggtctgtaaagatcgttatccgcccgacccactacgaaaagcctagtaactggccaagtgaacgcgaagttgcgctccgccacaaccccagtggtagctctggaagatggggctcgcaccacccccgtggtaacacggttgcctgcccgcgtgtgcttccgggttcggtctcgtgccgttcacttcaacttcacgcaaccagccaagagcctattgtgctgggacggttttcctccggggccgtgaatgctgctaatcccaacctccgagcgtgtgcgcacaatccagtgttgctacgtcgtaacgcgtaagttggaggcggaacagactactttcggtaccccgtgtttcctctcattttatttaatattttatggtgacaattgttgagatttgcgctcttgcaacgttgccattgaatattggcttatactatttggttgccttttacaaaacctctgatatacccagttcttacattgatctgcttgtttttctcaatttgaagtatagactacaaatagcaaa

SEQ ID NO: 502 - DNA, 675 bp, Enterovirus sp.
cgtggcggccagtactctggtatcacggtacctttgtacgcctgttttatatccccttcccccgcaacttagaagaaaacaaatcaagttcactaggagggggtacaaaccagtaccaccacgaacaagcacttctgtttccccggtgatgtcgtatagactgtaaccacggttgaaaacgattgatccgttatccgctcttgtacttcgaaaagcccagtatcaccttggaatcttcgatgcgttgcgctcagcactcaaccccagagtgtagcttaggtcgatgagtctggacactcctcaccggcgacggtggtccaggctgcgttggcggcctacctgtggtccaaagccacaggacgctagttgtgaacaaggtgtgaagagcctattgagctacaagagaatcctccggcccctgaatgcggctaatcctaaccacggagcaagggtacacaaaccagtgtatatcttgtcgtaacgcgcaagtctgtggcggaaccgactactttgggtgtccgtgtttccttttgtttttatcatggctgcttatggtgacaatctaagattgttatcatatagctgttggattggccatccggtaatttattgagatttgagcatttgcttgtttcttcaacaatttcacctattcattgcatttcagcagtcaaa

SEQ ID NO: 503 - DNA, 675 bp, Unknown
  Description of Unknown PV3 sequence
tacctttgtacgcctgttttatactccctcccccgcaacttagaagcatacaattcaagctcaataggagggggtgcaagccagcgcctccgtgggcaagcactactgtttccccggtgaggccgcatagactgttcccacggttgaaagtggccgatccgttatccgctcatgtacttcgagaagcctagtatcgctctggaatcttcgacgcgttgcgctcagcactcaaccccggagtgtagcttgggccgatgagtctggacagtccccactggcgacagtggtccaggctgcgctggcggcccacctgtggcccaaagccacgggacgctagttgtgaacagggtgtgaagagcctattgagctacatgagagtcctccggcccctgaatgcggctaatcctaaccatggagcaggcagctgcaacccagcagccagcctgtcgtaacgcgcaagtccgtggcggaaccgactactttgggtgtccgtgtttccttttattcttgaatggctgcttatggtgacaatcatagattgttatcataaagcgagttggattggccatccagtgtgaatcagattaattactcccttgtttgttggatccactcccgaaacgttttactccttaacttattgaaattgtttgaagacaggatttcagtgtcaca

SEQ ID NO: 504 - DNA, 675 bp, Enterovirus sp.
ctttgtacgcctgttttacatcccctcccccacgtaactttagaagcaattcaacaagttcaatagagggggtacaaaccagtatcaccacgaacaagcacttctgtttccccggtgattttacataagctgtgcccacggctgaaagtgaatgatccgttacccgctcgagtacttcgaaaagcctagtatcgctttgggatcttcgacgcgttgcgctcagcactctaccccgagtgtagcttaggctgatgagtctgggcattccccatcggcgacgatggcccaggctgcgttggcggcctacccatggctaacgccatgggacgctagttgtgaacaaggtgtgaagagcctattgagctactcgagagtcctccggcccctgaatgcggctaatcccaaccacggatcaggtgcctccaacccaggaggtggcctgtcgtaacgcgcaagtctgtggcggaaccgactactttgggtgtccgtgtttccttttatcttttaaatggctgcttatggtgacaatcatagattgttatcataaagcgaattggattggccatccggtgaaatacaaacacattatttacttgtttgttggatttactccgctcacacagcttactcctaagataatatttattgtattgctggtaaggagacactattata

SEQ ID NO: 505 - DNA, 636 bp, Enterovirus sp.
aagcaaggcaaacctgaccaatagtaggtgtggcacaccagccgcattttggtcaagcacttctgtttccccggaccgagtatcaataagctgctcacgcggctgaaggagaaaccgttcgttacccgaccagctacttcgagaaacctagtaacactatgaacgttgcggagtgtttcgttcagcacttcccccgtgtagatcaggtcgatgagtcaccgcattcctcacgggtgaccgtggcggtggctgcgttggcggcctgcctacgggttcgcccgtaggacgctctaataccgacatggtgtgaagagtccattgagctagctggtagtcctccggcccctgaatgcggctaatcctaactgcggagcaggtgctcacagaccagtgagtagcctgtcgtaacgggcaactctgcagcggaaccgactactttgggtgtttttccttttttcttctcttatattggctgcttatggtgacaattaaagaattgttaccatatagctattggattggccatccggtgacgagcagagccattgtttacctctttgttggatttgtacctttgaaccacaaagtcttgaataccattcatctcattttaaagttcaactcagctaaaagaaa

SEQ ID NO: 506 - DNA, 675 bp, Unknown
  Description of Unknown SA5 sequence
agtacttggtattccggtacctttgtacacctatttacaaaccctaccccttgtaaccttagaagcaattatttaaccgctcactagggggtgtgctatccaagcacatcaagagcaagcacttctgtctccccgggaggggctaatggtacgctgtgcccacggcggaaatgagccctaccgttaaccggcagtctacttcgggaagcccagtaactacattgaaactttgaggcgttacactcagcacataaccccaatgtgtagttctggtcgatgagccttggcatcccccacaggcgactgtggccaaggctgcgttggcggccagcctgcggaccaaaagtccgtaggacgcctaattgtggacatggtgtgaagagcctactgagctagactgtagtcctccggcccctgaatgcggctaatcctaaccctggagcatccgcgtgcaacccagtacgtagggtgtcgtaatgcgtaagtctgggatggaaccgactactttgggtgtccgtgtttcttgtttttcatactgggtcgcttatggttacaactaattgttgtaatcattggcagtgcgcgctgaccacgcgattattgatatttccatttgttggatactccaatagtgtcaactcatatacacaacttttaccactgatcaagataaaa

SEQ ID NO: 507 - DNA, 675 bp, Enterovirus sp.
tgtgcgcctgttttgaaaccccctcccccaactcgaaacgtagaagtaatgtacactactgatcagtagcaggcgtggcgcaccagccatgtctcgatcaagcacttctgtttccccggactgagtatcaatagactgctcacgcggttgaaggtgaaaacgtccgttacccggctaactacttcgagaaacctagtagcaccatagaaactgcagagtgtttcgctcagcacttcccccgtgtagatcaggtcgatgagtcactgcaatccccacgggtgaccgtggcagtggctgcgttggcggcctgcctatggggcaacccataggacgctctaaggtggacatggtgtgaagagtctattgagctagttagtagtcctccggcccctgaatgcggctaatcctaactgtggagcgcatactcccaaaccagggagcagtgcgtcgtaacgggcaactccgcagcggaaccgactactttgggtgtccgtgtttccttttattcctatactggctgcttatggtgacaattgagagattgttaccatatagctattggattggccatccagtgtgtaatagagcaatcatttaccaatttgttggatttactccattaacccacacgtctctcaacacactacatttcatcttactactgaacactagaaa

SEQ ID NO: 508 - DNA, 675 bp, Unknown
  Description of Unknown Mobovirus A sequence
tattctcccacaaaccttcttgtaactctgttaagccttttacatccatgtaatttaattttctccacctaaaaggatttcccccatggtcctttttggctcgaacaaatgctacatagggtcttgttttctccccctggctctcttgccagggttccataccccaattcctctatttccatgatttttcatcatggtttatttttactgtcttcttattttctgaggtgaccaactcctaagccgactgggtcgcggaagcccggactcctcgcatcactagggtgcgtagcgatgtaggcgaaaatattggttgctagatgcatacatatagtgaattgatactacaccaaactctgttctttttgaaactagctattttctaagtaaggtaggctacgggtgaaaccttaccattgcaggtacgtgaaccgcaacggacatttggccgaagactggtgtacccacgtcagttataggacctcttcaacgttggtggacggcatgtcactgattagttaggctagtgaatttaagttcagggggtatcttttagcttaagcgtgtattctagtaggacttgcagagcctccccacctaggaggatctctgtttatagccccttttccttgttccgttagttttccacacttttacaatatttgatgatttgtt

SEQ ID NO: 509 - DNA, 509 bp, Unknown
  Description of Unknown Burpengary Virus sequence
ctccccccccttccccttcccgagtaggagattggcatgtatgctctacatgcccgattctctcttgctcactctcttaaatcctggtggcggtctcggattaaacatttatgtcgtatctgggatcgtcttacttggtggtaattcctctgttgcctagggacctccggactgccggattaaaggtctcaacagagggcaatgtacaaggaagtcattatacgctaattaagtatttgatgaatgactagtgtgacagggctgaggaactccccccgggtaaccggtgcctcagcgtccgaaagacacgtggataggatccaccctgttatacccagcacgatgtaatagtcaaatacctctgatttgtgtaggatgtataaattgtgcattgtaaattttgggcgtagagatgctccgaaggtaccccgttttacgggatctgatcggaggctaattacccaatgcgccctaaataacttcatataatttctttttctttattcaaa

SEQ ID NO: 510 - DNA, 675 bp, Hunnivirus sp.
taacgtttggcaagaaccctcacctgtcaattgggaccaccactttcagtgaccccatgcgaagtagtgagagagaataagctttcttacccttcatttgtgaacccttcagtcgaagccgcttggaataagataggaggaaaagttcattctaaatggagtgaaacatgtacttcagaatttctagcacgcgctgggctttcttgcgtgtgacggcactgtcttgccggagctctccacactgacaccccacgcttgtggaccttggtggcagatgacaacactgcagctggaattgagtgtctggtacactctgtgtaacagtgaaaacaatgtgatcacttcggtgagctagtagcctgtggaccaacaactggtaacagttgcctcaggggccaaaagccacggtgtttacagcaccctactggtttgattggagcaatccaagatgtcacagagttagtaattgccaagcagtccgtactggtatcttgacataccgtgcagttttggatagtgaaggatgccctgacggtacccataggtaacaagtgacactatggatctaagcaggggctcactctacgctgctttacagctggctgtgagttaaaaaacgtctagctatccacaacctaggggactaggttttccttttatttagattacaattat

SEQ ID NO: 511 - DNA, 675 bp, Hunnivirus sp.
acagtttttgacaaggaccctcacctgtcaatcgggaccaccactttcagtgaccccgtgcgaagtgttgagagaaagtgagctttcttacccttcatttgtgaacccttcagtcgaagccgcttggaataagatggaaggaaatgttcattctaaatggagtgaaacatatacttaatttccagtgtttagtggtctttccactagacaacggcactgtcttgccggaactctacacaccaacattccacgcttgtgggactcaaatgttggatgacacagttgtagctggaactgagtgtttagtgcactctgtgtaacagtgaaaacaatgtgatcacttcggtgggctagtagcctgtggactaacaactggtaacagttgcctcaggggccaaaagccacggtgttaacagcaccctactagtttgattggagcaatccatgatgttacagagttagtaactgccaaacagattgtactggtatcttggcataccgtgcaacttaggatagtgaaggatgccctggcggtacccataggtaacaagtgacactatggatctaaacaggggctcactctacgttgctttacaactagctgtgagttaaaaaacgtctaactatccacaacctaggggactaggttttcctttttatttttatacacaacta

SEQ ID NO: 512 - DNA, 240 bp, Unknown
  Description of Unknown Ia Io sequence
ggtgtccgggtgtgtgggatagacccagatgtgcagtggatgcgagcatttgagtcagagtaggagcaagcccaggggcaaagggaccacattgtgtatcccgaatgaaggatcgagatttctctcctcattacccggtgtcttgtcactgttgggggggcccaacagtcttagtcctatactgcctgatagggtcgcggctggccggactcaagtgctatagtcagttgattttcactc

SEQ ID NO: 513 - DNA, 416 bp, Taura Syndrome Virus
ctttaaaagtcgtgcgtggcttcaccacgcacgatcagtactatcagttaaccactcttgaatatgctcaatgaccctattcaacactggtgtctcttagtacattattttagcacttaacgtgcatgagttttgcccatttctttcaaaaaaatgagtattcgaggagacgtcccgctccccgtcttatttcaaccgtagactcgacatctattggtggacatttaattccagtcgccgtaagttgcttctgccccgcgctatattttcttatacttatggttctataggtctggtttaaaacgtaaatagacggcccacaaactatagaacgcgtacccggaacgccaatcccggataagtccctggatatatagatgcaccgcaatataagcctgcagactgtctcatatact

SEQ ID NO: 514 - DNA, 604 bp, Acute bee paralysis virus
cccgtcaaaataacaacttataacacgatgttacccgaagaaaccattttagtgtaacatttaagattagaagtagttcatctaatagagataggcactattagaaggaggccttttctaaaggagccgttagtcagcccagacaagcgcagtactttagaagagagaagttccccgatagcgaccgaaaagacgcgttttccgtgctaactaatttaaatgtgggaacgaatattattattgaaattatgtgagccacgtagcaatcaagtcatgtttttgtcactacgtttactcatctaatgtagataattttgtttaagtacctatttaggtgtcatcccaccagagaagaaataatacgtaccggaacccagagtacaccccttattttaagccttactgggcttctctgttagttagtaatctggcccacgttttgcgttgagtggggtcccaacagtaggaattcgacggacaagtagcaagcgagtcggtaccaattggtttagccttcgaaattactctgggcaggaagttactaaacgagaactttctgcttaaatcccaacgcacaaacaaatagagtaaataaataattata

SEQ ID NO: 515 - DNA, 539 bp, Bovine rhinitis A virus
tttgttttgcggctttgccgttgttcgggttttacctgttttcacacagcaaaacaggccttctagtttcgtgcttaaacgagatcatgctcgaactagaactacatagctggtcactggactcataccacaccttgtggagctttatgggaaaggtggctagtgggctgtggaagtgactctgaccacatgcctctcaagtgtgggaaatcacggatcggtgtagcgacgacaacaggccttgggacaccctctccagtaatggagacccaaggggccaaaagccacgcctcgtgccctgttgttcacaaccccagtgcgacccgtgttagtacctatttgcgagaactgtgtctggacagctaaacacaaccctagtgggagactaaggatgcccaggaggtacccggaggtaacaagtgacactctggatctgacctggggagagagggcttgctttacaggcgcctctctttaaaaagcttctatgtctcatcaggcaccggaggccgggccttttccttttaaaattacactta

SEQ ID NO: 516 - DNA, 608 bp, Bovine rhinitis B virus
ccccccctacttaaagatgtacggttttgctgctttcacagagtaaagcagatagaggttctgaactggcaaactttacctcgaaacacgcccgtttttctgctgtgtctcacagactgtcctgtcacacttgtggcggcttgtgacactgtgaacatagtgagaccgaccaagacaacagtttcaagtgatgaacatcgaacgtctaaactggatccgtaactggacatgttagggcaaggacttcccccctggtaacaggagcctggctggccaaaagccccgctcattgagcctagcatgttgtcgaccctggactgttcagtttagttagtacatggaattcacttgtcacggttcttctgaactcggtctctagtatgacagcctaaggatgccctccaggtaccccggggtaacaagtgacacccgggatctgaggaggggactactttacgtagtttaaaaaacgtctaagctgttatggtgaccagaggctggcacctttcacttttaaaattacactactgactacaattgaagtgataacggttttacaggctttcaaactagttacacaagcactgttttcctgacacacacacttt

SEQ ID NO: 517 - DNA, 576 bp, Equine rhinitis A virus
aatattggcgcgcgcatttgcgcgcccccccccatttcagccccctgtcattgactggtcgaaggcgttcgcaataagactggtcgtcacttggctgttctatcgtttcaggctttagcgcgccctcgcgcggcgggccgtcaagcccgtgcgctgtatagcgccaggtaaccggacagcggcttgctggattttcccggtgccattgctctggatggtgtcaccaagctgacaaatgcggactgaacctcacaaagcgacacacctgtggtagcgctgcccaaaagggagcggaactcccccgccgcgaggcggtcctctctggccaaaagcccagcgttaatagcgccttttgggatgcaggagccccacctgccaggtgtgaagtggagtgagtggatctccaatttggtctgttctgaactacaccatctactgctgtgaagaatgccctggaggcaagctggttacagccctgaccaggggccctgcccgtgactctcgatcggcgcagggtcaaaaattgtctaagcagcagcaggaacgcgggagcgtttcttttccctttgtatcgac

SEQ ID NO: 518 - DNA, 466 bp, Unknown
  Description of Unknown GFTV sequence
atggggaagggtatgacgtgccccttccttcttcggagaactcgctctagtggtctttccacttctggaaaagagtgagtgcacgtgatcaggaccgtcgaagacgacaaatacctggtgctctatctcatagacgtttcacagctgtagcgacccctcagtagcagcggaagccccctcctggtgacaggagcctctgcggccaaaagccacgtggataagatccactgctgagggcggtgcgaccctagcaccctgtgatgcatactagttgtagcgtgccggactattggtctgtcataagacacctgatagagagaccaagaatgtcctggaggtaccccgcgtgcgggatctgaccaggagaccattgcccaatgctttacaacgggtctatggtttaaaaactgtcgcagtctctccaaaccaagtggtcttggttttcaattactttgaatatttcact

SEQ ID NO: 519 - DNA, 675 bp, Unknown
  Description of Unknown SAFV V13C sequence
ttttcgacgtggttggaattgccatcatttccgacgaaagtgctatcatgcctccccgattatgtgatgttttctgccctgctgggcggagcattctcgggttgagaaaccttgaatctttttctttggaaccttggttcccccggtctaagccgcttggaatatgacagggttattttcttgatcttatttctacttttgcgggttctatccgtaaaaagggtacgtgctgccccttccttctctggagaattcacacggcggtctttccgtctctcaacaagtgtgaatgcagcatgccggaaacggtgaagaaaacagttttctgtggaaatttagagtgcacatcgaaacagctgtagcgacctcacagtagcagcggactcccctcttggcgacaagagcctctgcggccaaaagccccgtggataagatccactgctgtgagcggtgcaaccccagcaccctggttcgatgatcattctctatggaaccagaaaatggttttctcaagccctccggtagagaagccaagaatgtcctgaaggtaccccgcgtgcgggatctgatcaggagaccaattggcggtgctttacactgtcactttggtttaaaaattgtcacagcttctccaaaccaagtggtcttggttttccaattttgttgaatggcaat

SEQ ID NO: 520 - DNA, 675 bp, Unknown
  Description of Unknown SAV P-113 sequence
ggagatctaagtcaaccgactccgacgaaactaccatcatgcctccccgattatgtgatgctttctgccctgctgggtggagcatcctcgggttgagaaaaccttcttcctttttccttggaccccggtcccccggtctaagccgcttggaataagacagggttatcttcacctcttccttcttctacttcatagtgttctatactatgaaagggtatgtgtcgccccttccttctttggagaacacgcgcggcggtctttccgtctctcgaaaagcgcgtgtgcgacatgcagagaaccgtgaagaaagcagtttgcggactagctttagtgcccacaagaaaacagctgtagcgaccacacaaaggcagcggaccccccctcctggcaacaggagcctctgcggccaaaagccacgtggataagatccacctttgtgtgcggcacaaccccagtgccctggtttcttggtgacacttcagtgaaaacgcaaatggcgatctgaagcgcctctgtaggaaagccaagaatgtccaggaggtaccccttccctcgggaagggatctgacctggagacacatcacatgtgctttacacctgtgcttgtgtttaaaaattgtcacagctttcccaaaccaagtggtcttggttttcactctttaaactgatttcact

SEQ ID NO: 521 - DNA, 612 bp, Unknown
  Description of Unknown VHEV sequence
aattccttcttcctttctccttggacctcggtcccccggtctaagccgctcggaatatgacagggttattttcacctcttctctcttctacttcatagtgttctatactatgaaagggtatgtgtcgccccttccttcttggagaacgtgcgtggcggtctttccgtctctcgaaaaacgtgcgtgcgacatgcagagtaacgcaaagaaagcagttcttggtctagctctggtgcccacaagaaaacagctgtagcgaccacacaaaggcagcggaaaccccctcctggtaacaggagcctctgcggccaaaagccacgtggataagatccacctttgtgtgcggtgcaaccccagcaccctggtttcttggtgacaccttagtgaaccctcgaatggcaatctcaagcgcctctgtaggaaagccaagaatgtccaggaggtaccccttcctcatggagggatctgacctggagacacatcacacgtgctatacacttgtgcttgtgtttaaaaattgtcacagctttcccaaaccaagtggtcttggttttcccttaacttcgaaaagtcactatggcctgcaaacatggatacccagacgtgtgccct

SEQ ID NO: 522 - DNA, 675 bp, Unknown
  Description of Unknown TRV NGS910 sequence
atgcgacgtggttggagattaaaccgactccgacgaaagtgctatcatgcctccccgattatgtgatgttttctgccctgctgggcggagcattctcgggttgatacaccttgaatccttcatccttggacctcaggtcccccggtctaagccgcttggaatacgacagggttattttccaatcttctcctttctactttcatgagtcctattcatgaaaagggtctgtgctgccccttccttcttggagaatctgcgcggcggtctttccgtctctcgaaaagcgcagatgcagcatgctggaaccggtgaagaaaacagttctttgtggaaacttagagcagacatcgaaacagctgtagtgacctcacagtagcagcggaaccccctcctggtaacaggagcctctgcggccaaaagccccgtggataagatccactgctgtgagcggtgcaaccccagcaccctggttcgatggttgttctctgtggaaccagagaatggtctttctcaagccctccagtagagaagccaagaatgtcctgaaggtaccccgcatgcgggatctgatcaggagaccaatcgtcagtgctttacactggcgctttggtttaaaaactgtcacagcttctccaaaccaagtggtcttggttttcacttttatcaaactgtttc

SEQ ID NO: 523 - DNA, 551 bp, Unknown
  Description of Unknown EMCV2 RD1338 sequence
aaatactggtcgaaaccgcttgggataagaccggggtttgttaatgtctcaatgttattctccacccaattgacgtcttttgtcaattggagggcagtgaaaccttgcccttgcttcttgcagaggattcccagtggtctttccgctctcgacaagggaattcatgatccaccaaaagttgtgaagagagcaggtcccatggaagctttctgacgactgatgatgactgtagcgaccctttgcaggcagcggacccccccacctggtgacaggtgcctctgcggccaaaagccacgtgtttaacagacacctgcaaaggcggcacaaccccagtgcctcatcaaaagtctgatgactgtggaaatagtcaaccggcttttcttaagcaaatttggtgtcggggctgaaggatgcccggaaggtaccacactggttgtgatctgatccggggccacagtacatgtgctttacacatgtagctgcggttaaaaaacgtctaggccccccgaaccacggggacgtggttttcctttgaaaaccacgattacaat

SEQ ID NO: 524 - DNA, 675 bp, Unknown
  Description of Unknown EMCV1 JZ1203 sequence
gtctgctcgatatcgcaggctgggtccgtgactacccactccccctttcaacgtgaaggctacgatagtgccagggcgggtactgccgtaagtgccaccccaaaacaacaacaaaccccccctaacattactggccgacgccgcttggaataaggccggtgtgcgtttgtctatatgttatttcccaccacattgccgtcttttggcaatgtgtgggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcggccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 525 - DNA, 544 bp, Unknown
  Description of Unknown EMCV1 AnrB-3741 sequence
atgtggtcgaagccacttggaataagaccggcgtgcgcttgtctatatgttacttccaccacattgccgtcttttggcaatgtgagggcccggaacctggccctgtcttcttgacgaacattcctaggggactttcccctctcgccaaaggaatgtaaggtctgttgaatgtcgtgaaggaagcagttcctctggaagcttcttgaagacaaacagcgtctgtagcgaccctttgcaggcagcggaaccccccacctggtaacaggtgcctctgcggccgaaagccacgtgtataagatacacctgcaaaggcggcacaaccccagtgccacgttgtgcgttggatagttgtggaaagagtcaaatggctttccccaagcgtattcaacaaggggctgaaggatgcccagaaggtaccccactggttgggatctgatctggggcctcggtgcaggtgctttacacctgttgagtcgaggttaaaaaacgtctaggccccccgaaccacggggacgtggttttcctagaaaaccactatgacaat

SEQ ID NO: 526 - DNA, 675 bp, Cosavirus sp.
cgtgctttacacggtttttgaaccccacaccggctgtttggcgcttgcaggacagtaggtatattttctttcatttctcttttctagccgcgtaggttctatctacgcgggcggagtgatactcccgctccttcttggacaggcggcctccacgctctttgtggatcttaaggctgccaagtcactggtgtttgaagtgaagaatggagagacactagggcgtttcatgtggctttgccagggattgtagcgatgctgtgtgtgtgtgcggatttcccctcgtggcgacacgagcctcacaggccaaaagccctgtccgaaaggacccacacagtggggttgccccgacccctcccttcaaagctttgtgtaaacaaacttttgtttagactttcttaagcttctctcacatcaggccccaaagatgtcctgaaggtaccctgtgtatctgaggatgagcaccaccaactacccggacttgtgggacgtgtcccacagacgcatgtggtattccagccccctccttttgaggagggggcttttgctcgctcagcacaggatctgatcaggagattcatctctggtgctttacaccagagcatggatttaaaaattgcccaaggcctggcaaacaacctaggggactaggttttctctattttaaaagatgtcaat

SEQ ID NO: 527 - DNA, 675 bp, Cosavirus sp.
cgtgctttacacggtttttgaaccccacaccggctgtttggcgcttgcaggacagcaggtttattttcttttaactctctctttctagccacacacgatctatgtgtgtgggcggagtgatactcccgttccttcttggacaggcggcctccacgccctttgtggatcttaaggctaccaagtcactggtgttggaaagtgaagagaaaggagttccttgggaactacatgtggcattgacagaggttgtagcgatgctgtgtgtgtgtgcggattacccccgtggcgacacggaccccacaggccaaaagccctgtccgaaaggacccacacagtggagcaaccccagctcccctcttcaatgttttgtgttagcaaccttggtattattttctctcaagcttccaatacaccgggccccaaagatgtcctgaaggtaccccgtgtatctgaggatgagcaccatcaactacccggacttgttctttcgagaacagacgcatgtggtaacccagccccgatcctaaggggtcggggcttttgctcactcagcacaggatctgatcaggagacctcccccccctgctttacagggggcgggggtttaaaaattgcccaaggcctggcaaataacctaggggactaggttttcctttttattttaaagttgtcaat

SEQ ID NO: 528 - DNA, 675 bp, Cosavirus A
ccgtgctttacacggtttttgaaccccacaccggctgtttggcgcttgcaggacagcaggtttattttcttatgctctttatttctagccaacagggttctatcctgttgggcggagtgatactcccgttccttcttggacagattgcctccacgatctttgtggatctcaaggtgatcaagtcactggtaaatagagcgaaggttgaggaaacctgaggaatttccatgtggttttgccaggagttgtagcgatgctgtgtgtgtgtgcggatttcccctcatggcaacatgagcctcacaggccaaaagccctgtccgaaaggacccacacagtggagcaatcccagctccctcctacaaagctttgtgagaatgaactcacgtttattcttctttattctctgtttacatcaggccccaaagatgtcctgaaggtaccttgtgtatctgggcatgagcaccatcaactacccggacttgcatttcggtgcagacacatgtggttacccagcccctctgctttggcagaggggcttttgctcgctcagcacgagatctgatcaggagcccttcccagtgtgctttacacctggcggggggttaaaaattgcccaaggcctggcaaaataacctaggggactaggttttccttttattaacaatgtctgtcatt

SEQ ID NO: 529 - DNA, 660 bp, Malagasivirus B
ctttattttcttatgtaactcttctttttaagttttattttgcctacttgtgagcttatgcgggaccactgtcttagacaaccccacatttgtcatgagtaagtacacgcaaccattacgattactttttaaccgtctgaccttttgataacaactgaagttaggcgtgaaacatgcatttataccaaagtagccccgcatttccccactacggtgggggggctaccctactggctttggaactgtagccattatgtgttgcctggctttcaggatctcacaacacaacagttctctcacaatggaatatgggtgagattgcagtgacatgaacaagtatctagtagtacatagactcaagcctagttgcctgcggaacaacatgtggtaacacatgccccagggtccaaaagacaagggttaacagccccttctaggtgtctgtgtgtgaagaatactttagtagtgttgttatgatctcacctgttagtacagaatgagtatggcttggtgaaggatgtcctacaggtacccattatatggatctgagtaggagaccactagtggtggctttaccgccaggtgagtggtttaaaaagcgtctagccaagccaacagcactagggatagtgctttctatattttatattttcagtgtat

SEQ ID NO: 530 - DNA, 654 bp, Mosavirus sp.
cccccccctcaaattgcaacgatatagctaatggcgagattgagatgctatatcacctcttctaagttatagacctcatctgattgataaggacgtaatttggtcgaaaccgcttggaataagaccgatgcgcgtagtcatgatgatgatgtaagatctaggaacttatccaatctgcttatgtctatgtaagtagaggggcaggcctcattgccctaattctttctaccgagtatctgctagggtttctagcggcttgaatacttggattgagggatacaagatactactgatcgattgtcgattgggaaacttgtagatacttcaaagctaccagtagcgtggactcacagccagcggactacccctcatggtaacatgagcctctgggcccacaaggcacgtcgcaagacctgtgagacggcaaccccagcctagctttgttgaggaaacaagcgataacatgacatgagagaccggaaggattcttgtattgtgagccgaaggatggcctctaggtacctcattttatgagatctgaggaggtgctcttgagttggtgctttacactgacaacacagagttaaaaagcgtctaagctcacccggaaattgggaaatttccgttatttccattttgtttgcaaagtcgttc

SEQ ID NO: 531 - DNA, 654 bp, Unknown
  Description of Unknown SVV sequence
ctgggccctcatgcccagtccttcctttccccttccggggggtaaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctatccactcactcacgcacaaaaagtgtgctgtaattacaagatttagccctcgcacgagatgtgcgataaccgcaagattgactcaagcgcggaaagcgctgtaaccacatgctgttagtcccttcatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaat

SEQ ID NO: 532 - DNA, 331 bp, Teschovirus A
actagtccttggacttttgttgtgtttaaacacagaaatttaattacctggccatgaattcattggattaacccttctgaaagacttgctctggcgcgagctaaagcgcaattgtcaccaggtattgcaccagtggtggcgacagggtacagaagagcaagtactcctgaccgggcaatgggactgcattgcatatccctaggcacctattgagatttctctggggcccaccggcgtggagttcctgtatgggaatgcaggactggacttgtgctgcctgacagggtcgcggctggccgtctgtactttgtatagtcagttgaaactcacc

SEQ ID NO: 533 - DNA, 416 bp, Teschovirus B
cttccttttaattcgtaactgataagtgatagtccttggaagctaggtatttgttacgctagttttggattatcttgtgcccaacatttgttttcgaacatatgttgtgtttaaacacagaaatctagtttctttggttatgagtttaatggaatatccttttgaaagacttgccttggcgcgggctagagcgcaattgtcaccaggtattgcaccaatggtggcgacagggtacagaagagcaagtactcctgactgggtaatgggactgcattgcatatccctaggcatctattgagatttctctggagcccaccagcatggagttcctgtatgggaatgcaggactggacttgtgctgcctgacagggtcgcggctggccgtctgtactttgtatagtcagttgaaactcatt

SEQ ID NO: 534 - DNA, 664 bp, Tottorivirus sp.
cccctttacgtaactgcaacttaaagagtaccctactgcattggatgtgtggtaaacttttacgcacacatttgtagtagtgttagttatgttctacctaatgagtatgcatgcacccgtcgaaacacgcttgtgataagataggtgagtccatgtgactaatctcattaagataaataagcaccctacaacgcacggcacgctcgtgtcttccgtgcggggccgggacaacagcggcctaaatcttctaggtgaccaccatgcttttgggactatggcaccactgtggacgtgagtacctggcagtaagtctgtgaaaagatggaaggtgtcccaagctatggggcgtatgcatatagcctgcggaacaaacaacggcgacgttgtccccagggcccaaaaggcacgtggataagatccacctatatgtttaccccatagtgtaagtcactggaagtcctagtaatggatgtctggagtaaggctcacggggtagggcgaaggatgcccagaaggtacccgtaggtaaccttaagagactatggatctgatctggggaccggatggcgccatcaccatgacgtggaggccggtttaaaaaacgtctaagcccgaccaacaacctaggggactaggttttccttttttattcatgtatgacgtt

SEQ ID NO: 535 - DNA, 675 bp, Unknown
  Description of Unknown Posavirus 1 sequence
acatttccttgcgtgcgcacccgaaaatttattgaacttggcttgaatcataagtaatgcttcttatagcggacactttgagaatataatgcttgtatggattaatgtatactgttttaaataacaaacctagacacgcagttgcgttgatggttgtatcaatcacatataagtgttgaactcgtgttaatcctcgatcgctatatgtttgccgcctacttccaataaaatagattacatgcgcgtcatgcctttgtgggttacctattggcctctgacaaaaacaagtcgtaagatttgtagcttcccggtgtaaaaagctgggcgcggtctggctctcgtagggtggaaaggtccaccaatggctggttgagtgtaagctccggtgtcctggttgtcgcaattccaggcgtcgtaataacctatattgcatctgactctaactcttgtggctctactgtatctagttcttgttctactaactctaataatactactggctctaatactgaaaaacttacatatgttaatatagataatatccttgatcctgatatccctcacgtcactgaagttcgccgaaaacgaatttcagatcatatcattgaatctcaaggatgtacttgctctgaacctactataactcctcatgcgttttcattttctactcttggc

SEQ ID NO: 536 - DNA, 675 bp, Unknown
  Description of Unknown A105-675 sequence
ccacccacagcaagaatgccatcatctgtcctcacccccatttctcccctccttcccctgcaaccattacgcttactcgcatgtgcattgagtggtgcacgtgttgaacaaacagctacactcacgtgggggcgggttttcccgcccttcggcctctcgcgaggcccacccttccccttcctcccataactacagtgctttggtaggtaagcatcctgatcccccgcggaagctgctcgcgtggcaactgtggggacccagacaggttatcaaaggcacccggtctttccgcctccaggagtatccctgctagtgaattctagtggggctctgcttggtgccaacctcccccaaatgcgcgctgcgggagtgctcttccccaactcaccctagtatcctctcatgtgtgtgcttggtcagcatatctgagacgatgttccgctgtcccagaccagtccagcaatggacgggccagtgtgcgtagtcgtcttccggcttgtccggcgcatgtttggtgaaccggtggggtaaggttggtgtgcccaacgcccgtactttggtgacaactcaagaccacccaggaatgccagggaggtaccccgcctcacggcgggatctgaccctgggctaattgtctacggtggttcttcttgcttccatttctttcttctgttc

SEQ ID NO: 537 - DNA, 675 bp, Unknown
  Description of Unknown A110-675 sequence
acctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 538 - DNA, 675 bp, Unknown
  Description of Unknown 18-675 sequence
cccacagcaagaatgccatcatctgtcctcacccccaattttcccttttcttcccctgcaaccattacgcttactcgcatgtgcattgagtggtgcatgtgttgaacaaacagctacactcacatgggggcgggttttcccgccctacggcctctcgcgaggcccaccccttccctccccttataactacagtgctttggtaggtaagcatcctgatcccccgcggaagctgctcacgtggcaactgtggggacccagacaggttatcaaaggcacccggtctttccgccttcaggagtatccctactagtgaattctagcggggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgcgtgtagtcgtcttccggcttgtccggggcatgtttggtgaaccggtggggtaaggttggtgtgcccaacgcccgtactttggtgacacctcaagaccacccaggaatgccagggaggtaccccacctcacggtgggatctgaccctgggctaattgtctacggtggttcttcttgcttccacttctttcttctgttcacg

SEQ ID NO: 539 - DNA, 675 bp, Unknown
  Description of Unknown A115-675 sequence
acctttgtgcgcctgttttataccccccccaacctcgaaacttagaagtaaagcaaacccgatcaatagcaggtgcggcgcaccagtcgcatcttgatcaagcacttctgtaaccccggaccgagtatcaatagactgctcacgcggttgaaggagaaaacgttcgttacccggctaactacttcgagaaacccagtagcatcatgaaagttgcagagtgtttcgctcagcactacccccgtgtagatcaggccgatgagtcaccgcacttccccacgggcgaccgtggcggtggctgcgttggcggcctgcctatggggcaacccataggacgctctaatacggacatggtgcgaagagtctattgagctagttagtagtcctccggcccctgaatgcggctaatcctaactgcggagcacatacccttaatccaaagggcagtgtgtcgtaacgggtaactctgcagcggaaccgactactttgggtgtccgtgtttccttttaatttttactggctgcttatggtgacaattgaggaattgttgccatatagctattggattggccatccggtgactaacagagctattgtgttccaatttgttggatttaccccgctcacactcacagtcgtaagaacccttcattacgtgttatttctcaactcaagaaa

SEQ ID NO: 540 - DNA, 675 bp, Unknown
  Description of Unknown A73-675 sequence
ttactccattcagcttcttcggaacctgttcggaggaattaaacgggcacccatactccccccaccccccttttgtaactaagtatgtgtgctcgtgatcttgactcccacggaacggaccgatccgttggtgaacaaacagctaggtccacatcctcccttcccctgggagggcccccgccctcccacatcctccccccagcctgacgtatcacaggctgtgtgaagcccccgcgaaagctgctcacgtggcaattgtgggtccccccttcatcaagacaccaggtctttcctccttaaggctagccccggcgtgtgaattcacgttgggcaactagtggtgtcactgtgcgctcccaatctcggccgcggagtgctgttccccaagccaaacccctggcccttcactatgtgcctggcaagcatatctgagaaggtgttccgctgtggctgccaacctggtgacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccacctccgggtgggatctgagcctgggctaattgtctacgggtagtttcatttccaatccttttatgtcggagtc

SEQ ID NO: 541 - DNA, 675 bp, Kobuvirus sp.
ttactccattcagcttcttcggaacctgttcggaggaattaaacgggcacccatacacccccatcccctttctgcaacttaagtatgtgtgctcgtgatcttgactcccacggaatggatcgatccgctggagaacaaactgctagatccacatcctcccttcccctgggaggaccttggtcctcccacatcctccccccagcctgacgtaccacaggctgtgtgaagcccccgcgaaagctgctcacgtggcaattgtgggtccccccttcatcaagacaccaggtctttcctccttaaggctagccccgatgtgtgaattcacattgggcaactagtggtgtcactgtgcgctcccaatctcggccgcggagtgctgttccccaagccaaacccctggcccttcactatgtgcctggcaagcatacctgagaaggtgttccgctgtggctgccagcctggtaacaggtgccccagtgtgcgtaaccttcttccgtcttcggacggtagtgattggttaagatttggtgtaaggtccatgtgccaacgccctgtgcgggatgaaacctctactgccctaggaatgccaggcaggtaccccacccccgggtgggatctgagcctgggctaattgtctacgggtagtttcatttccaattcttttatgtcggagtc

SEQ ID NO: 542 - DNA, 675 bp, Aichivirus sp.
ttactccattcagcttcttcggaacctgttcggaggaattaaacgggcacccatacatccccatcccctttctgtaacttaagtatgtgtgcttgtaatcttgactcccacggaatggatcgatccgctggagaacaaactgctagatccacatcctcccttcccctgggaggaccttggtcctcccacatcctccccccagcctgacgtaccacaggctgtgtgaagcccccgcgaaagctgctcacgtggcaattgtgggtccccccttcatcaagacaccaggtctttcctccttaaggctagccccgatgtgtgaattcacattgggcaactagtggtgtcactgtgcgctcccaatctcggccgcggagtgctgttccccaagccaaacccctggcccttcactatgtgcctggcaagcatatctgagaaggtgttccgctgtggctgccagcctggtaacaggtgccccagtgtgcgtaaccttcttccgtctccggacggtagtgattggttaagatttggtgtaaggttcatgtgccaacgccctgtgcgggatgaaatctctactgccctaggaatgccaggcaggtaccccaccctcgggtgggatctgagcctgggctaattgtctacgggtagtttcatttccaatccttttatgtcggagtc

SEQ ID NO: 543 - DNA, 675 bp, Aichivirus sp.
actccattcagcttcttcggaacctgttcggaggaattaaacgggcacccatacacccccatccccttttttgcaacttaagtatgtgtgctcgtaatcttgactcccacggaatggatcgatccgctggagaacaaactgctagatccacatcctccctcccccctgggaggacctcggtcctcccacatcctccccccagcctgacgtatcacaggctgtgtgaagcccccgcgaaagctgctcacgtggcaattgtgggtccccccttcatcaagacaccaggtctttcctccttaaggctagtcccgatgtgtgaattcacatcgggcaactagtggtgtcactgtgcgctcccaatctcggccgcggagtgctgttccccaagccaaacccctggcccttcactatgtgcctggcaagcatatctgagaaggcgttccgctgtggctgccagcctggtaacaggtgccccagtgtgcgtaaccttcttccgtccccggacggtagtgattggttaagacttggcgtaaggttcatgtgccaacgccctgtgcgggatgaaacctctactgccctaggaatgccaggcaggtaccccaccttcgggtgggatctgagcctgggctaattgtctacgggtagtttcatttctaattctttcatgtcggagtc

SEQ ID NO: 544 - DNA, 675 bp, Aichivirus sp.
ttactccattcagcttcttcggaacctgttcggaggaattaaacgggcacccatacacccccaccccctttttgcaacttaagtatgtgtgctcgtgatcttgactcccacggaatggatcgatccgctggagaacaaactgctagatccacatcctcccttcccttgggaggacctcggtcctcccacatcctccccccagcctgacgtaccacaggctgtgtgaagcccccgcgaaagccgctcacgtggcaattgtgggtccccccttcattaagacaccaggtctttcctccttaaggctagtcccgatgtgtgaattcacattgggcaactagtggtgtcactgtgcgctcccaatctcggccgcggagtgctgttccccaagccaaacccctggcccttcactatgtgcctggcaagcatatctgagaaggtgttccgctgtggctgccagcctggtaacaggtgccccagtgtgcgtaaccttcttccgtcttcggacggtagtgattggttaagatttggcgtaaggttcatgtgccaacgccctgtgcgggatgaaacctctactaccctaggaatgccaggcaggtaccccaccctcgggtgggatctgagcctgggctaattgtctacgggtagtttcatttccaattcttctatgtcggagtc

SEQ ID NO: 545 - DNA, 675 bp, Aichivirus sp.
tactccattcagcttcttcggaacctgttcggaggaattaaacgggcacccatacacccccacccccttttctgcaacttaagtatgtgtgctcgtaatcttgactcccacggaatggatcgatccgctggagaacaaactgctagatccacatcctcccttcccctgggaggaccccggtcctcccacatcctccccccagcctgacgtatcacaggctgtgtgaagtccccgcgaaagctgctcacgtggcaattgtgggtccccccttcatcaagacaccaggtctttcctccttaaggctagccccgatgtgtgaattcacattgggcaactagtggtgtcactgtgcgctcccaatctcggccgcggagtgctgttccccaagccaaacccctggcccttcactatgtgcctggcaagcatatctgagaaggtgttccgctgtggctgccagcctggtaacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccaccttcgggtgggatctgagcctgggctaattgtctacgggtagtttcatttccaattcttttatgtcggagtc

SEQ ID NO: 546 - DNA, 664 bp, Aichivirus sp.
gcttcttcggaacctgttcggaggaattaaacgggcacccatacacccccacccccttttctgcaacttaagtatgtgtgctcgtaatcttgactcccacggaatggatcgatccgctggagaacaaactgctagatccacatcctcccttcccctgggaggaccccggtcctcccacatcctccccccagcctgacgtatcacaggctgtgtgaagtccccgcgaaagctgctcacgtggcaattgtgggtccccccttcatcaagacaccaggtctttcctccttaaggctagccccgatgtgtgaattcacattgggcaactagtggtgtcactgtgcgctcccaatctcggccgcggagtgctgttccccaagccaaacccctggcccttcactatgtgcctggcaagcatatctgagaaggtgttccgctgtggctgccagcctggtaacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccaccttcgggtgggatctgagcctgggctaattgtctacgggtagtttcatttccaattcttttatgtcggagtc

SEQ ID NO: 547 - DNA, 675 bp, Aichivirus sp.
tactccattcagcttcttcggaacctgttcggaggaattaaacgggcacccactttcctgtcctctccccttttctgtaactccaagtgtgtgctcgtaatcttgactcccgcggattgaccgctccgctggtgaacaaactgctaggtcatctcctccccacccttgggcgtccttccgggcgtccacaccctccccccagcctgacgtgtcacaggctgtacaaagaccccgcgaaagctgctaacgtggcaattgtgggtcccccctttgtaaaggaaccgagtctttctcccttaaggctagacccctgtgtgaattcacaggtggcaactagtggttccactgcatgctcccgacctcggccgcggagtgctgttccccaagtcgtaacactgaccttcacttatgtgcctggcaagcatatctgagaagatgttccgctgtggctgccaaacctggtaacaggtgccccagtgtgcgtagtcttcttccgtcttcggacggtaggtgttaggtaaagatgcggcgtaaggttcaagtgccaacgccctggaagggatgacccttctactgccctaggaatgccgcgcaggtaccccaggttcgcctgggatctgagcgcgggctaattgtctacgggtagtttcatttccctcttcttccactggcatc

SEQ ID NO: 548 - DNA, 675 bp, Aichivirus sp.
actccattcagcttcttcggaacctgttcggaggaattaaacgggcacccactttcctgtcctctccccctttctgcaactcaagtgtgtgctcgtaatcctgactcccacgggttgaccgccccgttggtgaacaaacagctaggtcattccctccctacccctgggcgccatttcagtggcgttcatatcctccccccagcctgacgtgtcacaggctgtgcaaagtccccgcgaaagctgctcacgtggcaattgtgggtcccccctttgtgaaggaaccgagtctttctcccttaaggctagacccctgtgtgaactcacaggtggcaactagtggttccactgcatgctcccgacctcggccgcggagtgctgttccccaagtcgtgacactgacctccacttatgtgcctggcaagcatatctgagaagatgttccgctgtggctgccaaacctggtaacaggtgccccagtgcgtgtagtcttcttccgtctccggacggtaagtgtgtggtaaagatgcggcgtaaggttcaagtgccaacgccctggaagggatgacccttctactgccctaggaatgccgcgcaggtaccccaggttcgcctgggatctgagcgcgggctaattgtctacgggtagtttcatttccctctcttttcactggcatc

SEQ ID NO: 549 - DNA, 498 bp, Pestivirus sp.
gtataagggttgggaaccttgtaccaagctacctctgccattcagtatttgggagtagaagtagatgtgtttacaaactcacacgtgtgggggcggggatagactgtgccagcggtcgtgtaccagcacctacgcatacgtgtggactgcgaaccaggagagcacctaggtctgacaagctgtgagaacacagtagtcgtcagtgagtcagctggtaaggatcacccacctggatactcacgtggacgagggagtttcccagtcagaaacctacaccagaggaggggtcctctggagacatggatggtctgagtaacagactatctactggggtgtgctgcctgacagggtctcggctgatagcctggctagcagtataaaaatcagttgaattggcatatgagttgtgaacatctagtaaacaatgaaagacaaaaacaaaaaatgagcataataaaaaaattgtacaatccactactcaggtgtggctgcagactt

SEQ ID NO: 550 - DNA, 576 bp, Kobuvirus sp.
tttgaaaagggggtgggggggcctcggccccctcaccctcttttccggtggccattcgcccgggccaccgttactccactccactccttcgggactggtttggaggaacacaacagggcttcccatccctgtttaccctttattccatcatcctttccccaagtttaccctatccacaccccactgactgactcctttggattttgacctcagaatgcctatttgacctcccactcgcctctcccttttcggattgccggtggtgcctggcggaaaaagcacaagtgtgttgcaggctaccaaactcctacccgacaaaggtgcgtgtccgcgtgctgagtaatgggataggagatgcctacaacaggctcgcccatgagtagagcatggactgcggtgcatgtgacttcggtcaccacgggcatagcattgctcacccgtgaatcaagtcatcgagatttctctgacctctgaagtgcactgtggttgcgtggctgggaatccacgcttgaccatgtactgcttgatagagtcgcggctggccgactcatgggttaaagtcagttgacaagacac

SEQ ID NO: 551 - DNA, 675 bp, Kobuvirus sp.
cctacccaagggttacatgggaccatattcctcctcccctgtaactttaagttttgtgcccgtattcttgactccaggcggatgttgtgtcgcccgtcctgtgaacaaacagctagacactttcctcccctccctctgggctgctccggcagtccactccctccccccagcgtaacatgccccgctggagtgatgcacctggaagtcgtggacgtgggttagtaacttcggtgaaaacccactataatgacaactggttgacccccacactcaaaggactcgagtctttctcccttaaggctagcccggccacatgaatttgcagctggcaactagtgagtccaccatgtcccgcaacctcggctgcggagtgctgttccccaagcgtatgccttccttctgtaagagtgcgcctggcaagcacatctgagaagtcgttccgctgcgtcgtgccaacctggcgacaggtgacccagtgtgcgtagacttcttccggattcgtccggctcttctctaggaaacatgcgtgtaaggttcatgtgccaaagccctgcgcgcggtgttcttctactgccctaggaatgtgccgcaggtacccctacttcggtagggatctgagcggtagctaattgtctacgggtagtttcatttccatcttctcttcaggtcgacatc

SEQ ID NO: 552 - DNA, 675 bp, Kobuvirus sp.
gaccttctggtacttcttcgcctgggtcacaaaagcgaagaacctgcctctctaacgccagacgagcggcattaaacttgaacttctggcactctccactctcccttttccctgtccctttccccactgcgctctcaaggtcgcgcaatcctgggactagcccagttttaaaagttcctggcaccctttgcccctctaggcccttaaggtaggaactgaccttgtgctgtgatctcggtgcgggagtgctaccacgtagtcatcgtaagcctcgtttctggttctgccctggcaaggctacagagtaccgtgttccgctgtggatgccatccgggtaaccggacccccagtgtgtgtagcggtatgttcacggtccgccgtgttcaccagattcctgacctggctttgctagaaatggtgtgtgcccaatccctgtgaccagtatcaattacatcacctaggaatgctaggaaggtaccccagtcctgagctgggatctgatcctaggctaattgtctacggtgatgctccttttattttcttacaactgctattgactgtctgattgctgattctgctcttgtgctcttctgctctggctcattctcaagggttctctttgtccaagatcctttggttctctccttgttccacttgccactgccaacgcttgtc

SEQ ID NO: 553 - DNA, 369 bp, Pestivirus sp.
gtatacgcagttagttcatcctgtgtatacagattggagactctaaaaacaacgattcggaataggggcccgcggcgaagaccgaagacaggctaaccatgccgttagtagggctagcaccaaaacgcgggaactagacacttaggagagtggtctggctactctaagaggtgagtacaccttaaccgtcaagggttctactcctcagttgaggactagagatgccctgtggacgggggcatgcccaagagttagcttagccggggcgggggttgttccggtgaaagtagcaatattgaccacactgcctgatagggcggagcaggccccctaggtagtctagtataaaatgtctgctgtacatggcac

SEQ ID NO: 554 - DNA, 399 bp, Pestivirus sp.
tacgcggggtataacgacagtagttcaagtgtcgttatgcatcattggccataacaaattatctaatttggaatagggacctgcgacctgtacgaaggccgagcgtcggtagccattccgactagtaggactagtacaaataggtcaactggttgagcaggtgagtgtgctgcagcggctaagcggtgagtacaccgtattcgtcaacaggtgctactggaaaggatcacccactagcgatgcctgtgtggacgaggacatgtccaagccaatgttatcagtagcgggggtcgttactgagaaagctgcccagaatgggtagttgcacatacagtctgataggatgccggcggatgccctgtattttgaccagtataaatattatccgttgtaaagcat

SEQ ID NO: 555 - DNA, 123 bp, Pestivirus sp.
gcagatatcggtggtggacctgggggttgggctcaccgtgccccttcatggggtagacctcactgcttgatagagtgccggcggatgcctcaggtaagagtataaaatccgttgttcactaac

SEQ ID NO: 556 - DNA, 382 bp, Pestivirus sp.
gtatacgagtttagctcaatcctcgtatacaatattgggcgtcaccaaatatagatttggcataggcaacaccccgatgcgaaggccgaaaagggctaaccatgcccttagtaggactagcaaaaaatcggggactagcccaggtggtgagcttcctggatgaccgaagccctgagtacagggcagtcgtcaacagttcaacacgcagaataggtttgcgtcttgatatgctgtgtggacgagggcatgcccacggtacatcttaacctatccgggggtcggataggcgaaagtccagtattggactgggagtacagcctgatagggtgttgcagagacccatctgataggctagtataaaaaactctgctgtacatggcac

SEQ ID NO: 557 - DNA, 373 bp, Unknown
  Description of Unknown Classical swine fever virus sequence
gtatacgaggttagttcattctcgtatgcatgattggacaaattaaaatttcaatttggatcagggcctccctccagcgacggccgaactgggctagccatgcccacagtaggactagcaaacggagggactagccgtagtggcgagctccctgggtggtctaagtcctgagtacaggacagtcgtcagtagttcgacgtgagcagaagcccacctcgatatgctatgtggacgagggcatgcccaagacacaccttaaccctagcgggggtcgctagggtgaaatcacaccacgtgatgggagtacgacctgatagggtgctgcagaggcccactattaggctagtataaaaatctctgctgtacatggcac

SEQ ID NO: 558 - DNA, 222 bp, Pegivirus sp.
tcagggttggtaggtcgtaaatcccggtcaccttggtagccactataggtgggtcttaagagaaggttaagattcctcttgtgcctgcggcgagaccgcgcacggtccacaggtgttggccctaccggtgggaataagggcccgacgtcaggctcgtcgttaaaccgagcccgtcacccacctgggcaaacgacgcccacgtacggtccacgtcgcccttca

SEQ ID NO: 559 - DNA, 527 bp, Pegivirus sp.
cccggcactgggtgcaagccccagaaaccgacgcctatttaaacagacgttatgaaccggcgccgacccggcgaccggccaaaaggtggtggatgggtgatgccagggttggtaggtcgtaaatcccggtcatcttggtagccactataggtgggtcttaagggttggtcaaggtccctctggcgcttgtggcgagaaagcgcacggtccacaggtgttggccctaccggtgtgaataagggcccgacgtcaggctcgtcgttaaaccgagcccattacccacctgggcaaacaacgcccacgtacggtccacgtcgccctacaatgtctctcttgaccaataggctttgccggcgagttgacaaggaccagtgggggctgggcgacgggggtcgtataggaagaaaaatgccacccgccctcacccgaaggttcttgggctaccccggctgcaggccgccgcggagctggggtagcccaagaaccttcgggtgagggcgggtggcatttttcttcctataccgatc

SEQ ID NO: 560 - DNA, 402 bp, Pegivirus sp.
tggtcaccttggtagccactataggtgggtcttaagagaaggttaagattcctcttgtgcctgcggcgagaccgcgcacggtccacaggtgttggccctaccggtgtgaataagggcccgacgtcaggctcgtcgttaaaccgagcccatttcccgcctgggcaaacgacgcccacgtacggtccacgtcgcccttttaatgtctctcttgaccaataggttcatccggcgagttgacaaggaccagtgggggccgggggtcacagggatggaccctgggccctgcccttcccggcggggtggggaaagcatggggccacccagctccgcggcggcctgcagccggggtagcccaagaaccttcgggtgagggcgggtggcatttttcttcctataccgatc

SEQ ID NO: 561 - DNA, 531 bp, Unknown
  Description of Unknown Hepatitis GB virus A sequence
gccgggtggaaggcccggaaccgccccaccacctcaactaggtggtaagggtacgtctatcggtccggctggcccgaaaggcggtggatcctgtgtgttagggttcgtaggtggtaaatcccagcacaggtggtaatcgctatagggcaggcttatcccggtgaccgcttccctggatcctggagcgggtcgtggcggcacggtccacaggagtggggcctccggtgtgaataagccctcgtctggagcatcagacgttaaactgagacgtcccgaagagatcggaacgacgccccacgtatggcaacgccgcttaaaacccttcggggacagctatgcgggttgacaatgccagtggggggccgggcccactattgttgtgggctccgagttcctctagggatggccgaaaggcagccatggggccacccaggcggcgccgtgctacaggcggcaaggggaaaaatccttcgggtgaccccgggtggcattccctcccttagcagcatgagtgtggtggtagctgcaacc

SEQ ID NO: 562 - DNA, 520 bp, Pegivirus sp.
ggggaatctcaccccccgtccggttccggaagaatcggaaaccgacaccctgaccaatcattcttgatcatagagtggatgttagtgaaagccagacgaaagccggcggatgggtggtgacagggttggtaggtcgtaaatcccggccaccctggtacccggtataagttgggcggaagctgactgaagctccgtgctcttttctgtgcgttcttggtgcacggtccacaggtgacgcctataccggtgtgaataataggccgactcgagcggagtcgttaaactgagaacctccatacggatggcaacttggcttgcgtacggggacgccgctaaagtcacagtgggttaagtccggcgggttgacaaccccagcaaggcgagggggtcctattgttggactctgccagttcccggtggaggtaggcatggggtggcccagctccgcggcgcgctacagccggggtagcccaaaatccgaaaggtgagggcgggccacatgtccgaaatttagtcaagc

SEQ ID NO: 563 - DNA, 524 bp, Pegivirus I
agaatggtctaagtggttgccaccgtggtccgaaggggaggaggacctacgctgccagggttggcaggtcgtaaatcccgggtgtaggagatccctccttgttaggactgctggtagctggggggtcggtgaccccctgggcaaccgccaaacccggacgaccgggtggcggctccatgttggcacggtccacaggtgtgaaccctaccggtgtgaataagggttggtggttgcggtccaccttaaacgtagtatgcattgggcttggtaaaacaccgctcgtagtacggaacgccgcctttaaagacacagtaggcgtagccggcgggttgacaatccatacggggggtggggtgtggtcatggatctgtccacaccaccttcatgcggccctctaagcaagccataccggggggaggcgcgcggcaccgcactgccgggcaaggggaagaaccttcgggtgacccccccccaaccaccgtccgatcaatgctaatgttgcgtttaggcgtgacaccggcaca

SEQ ID NO: 564 - DNA, 537 bp, Pegivirus K
agaatggtgtgatccgtcgccgctccagcggaaagcgggcgggatctagtggttagggttgttcgcgtaaatcccacactagtggtacgctcgtataacgtgggagcagccggtggggtcgaccccccacctggcggctgctgagcaccggacgaagcgcggggggtgaacgctaacccgcggcccgggctgccaacgttaggcacgtcttggctggaagacgttaaacacagggccccccctcaaccctgatccgaggccagagaccaaggtacgccgcccttttaaaggcgttactcgtccaataggatctctccggcgggttgtcaaaccttgctggccctggtgatggttacgggagggggtggggcggggagtagaagccccgcccggcatgggggtaccaagctcggcacgcccagcacgcgtggcgtaggggaaaaatccttcgggtgacccctggtaccataaagtaattaacatgagcatgccgctagggtgtgctttttcttccttccttgggaaggcggtggcacc

SEQ ID NO: 565 - DNA, 617 bp, Unknown
  Description of Unknown Theiler′s disease-associated virus sequence
tgataccgtgtcccggtacgacctcgcgcgtccccaagctcgccctgaggggggagcgtaagggcgcgtagtggggtagccccccaaaccgagccaccctagtgagtgactttagaatggttagggagactaccgccttcgctgtttggggacctaatgatccgcgtgccagggttcttcgggtaaatcccggcgcggtgttttgggttcagggcagtaggggcagacgggccagcagtcgctggttcctggtaccaccaccctatccggacgacctccctcacgaaaggtcgccacggtctgtggctcgacgacgcctataattcagtccgaggggcgcagccctcgttaaacttaggcaaggttcctcgccattgatttggccaggggtttaagtgaacgccgcccttttaatgtttaatagggttctttcccggcgggttgacaaacacttccctgggctcttcgttggcctcggttccttgatgcttcggcacccatgagcgcacaggggggggaccctgcgacagtccgccaagaggaaaatccttcgggtgacctcgtgcgcaacccaatcccttcttcttccacatggcgtgtctgtggtgcatgctgtg

SEQ ID NO: 566 - DNA, 349 bp, Pegivirus sp.
ggacttcggtccccctgttactctgcgagccaccgcagagccagggttggtacgcccgaggtgttagaccccggccgaaagctcctaaccatggggttagtaggacgtggtaaatgccactgaggggttggagagctggtagagcgagtaagtcggcgtaaggcccgagtacgggcctcccagcccgggtcagcctaaacctggctgtgatacccggtgcatggagggcgtgtcccaacgctcgatcgctgtagggtgggtccctgcagttgggtgtggctaccctgctcgtactgcttgatagagtcccggcggacggaccagctctcgtcagtccgtggagttgcac

SEQ ID NO: 567 - DNA, 327 bp, Pegivirus sp.; identical to SEQ ID NO: 35

SEQ ID NO: 568 - DNA, 436 bp, Unknown
  Description of Unknown GB virus C/Hepatitis G virus sequence
cccccggcactgggtgcaagccccagaaaccgacgcctatctaagtagacgcaatgactcggcgccgactcggcgaccggccaaaaggtggtggatgggtgatgacagggttggtaggtcgtaaatcccggtcaccttggtagccactataggtgggtcttaagagaaggttaagattcctcttgtgcctgcggcgagaccgcgcacggtccacaggtgttggccctaccggtgggaataagggcccgacgtcaggctcgtcgttaaaccgagcccgttacccacctgggcaaacgacgcccacgtacggtccacgtcgcccttcaatgtctctcttgaccaataggcgtagccggcgagttgacaaggaccagtgggggccgggggcttggagagggactccaagtcccgcccttcccggtgggccgggaaatgc

SEQ ID NO: 569 - DNA, 393 bp, Pegivirus sp.
agaatggggagttaactcctggcactggcccgaagcatgaactgatcgcggtggcagggttcttcgggtaaatcccggccgcgtgttgtgattgtgttagggcaggtgacagtcggcagggtcgaccccctgcttcaggaccactgtcttcctggacgaccgttgctgaaaaagggccgccacggtctgtagctcgccgacgcttctaattcaggccggaggaccacgctccgtaatcgagcccaagtactcaaaccccagcacccctgggtcacgccctacgccgcccttttaacgcttcggctaatagggtctatccggcgggttgacaaagggcgcagggttacctggtactacgagcttgggtgtccctgggagtaatcccagggtgcc

SEQ ID NO: 570 - DNA, 343 bp, Flavivirus sp.
atataaatcccagtttggttaaacctatttcaaggcttaagttgtttattattttatcgccgctcgtgactataaagttgcctagcggagagagataaagaagaaggagttcaaggctcagggcagggcgcaagttccctggtccctaggccgctcgcaggaaggaggagtgaagaagaagaaagagaaggagaggaccaccgccgaaagaaggcaggtgcctcacaagagggccaaccagcgtgttggaccagtggccaacgccggacggcgtggtggcctgctgggacgcctggggattggatggagtgccttcctacaggaagacatcgttcaagccatc

SEQ ID NO: 571 - DNA, 104 bp, Unknown
  Description of Unknown Bussuquara virus sequence
agtatttcttctgcgtgagaccattgcgacagttcgtaccggtgagttttgacttaacgcagtgagaaaagttttcgaggaaagacgagaagcgaattctctga

SEQ ID NO: 572 - DNA, 106 bp, Zika virus
agttgttgatctgtgtgagtcagactgcgacagttcgagtctgaagcgagagctaacaacagtatcaacaggtttaatttggatttggaaacgagagtttctggtc

SEQ ID NO: 573 - DNA, 150 bp, Yokose virus
agtaaattttgcgtgctagtcgctgagcgtcagaccgcaaagtgagtttttagtgatctaaagtgaggagttattcttactgtcatcaaacactacaaataaacacgttgaaattatttccggaagaacaactgtccggaatcaaagacg

SEQ ID NO: 574 - DNA, 118 bp, Wesselsbron virus
agtatattctgcgtgctaatcgttcgacgttagtccgtggagtgagcttctattagagtcgttaacacgtttgaataatttctactgaaaggagtagaagaaaggagattcattccca

SEQ ID NO: 575 - DNA, 389 bp, Hepacivirus sp.
acctccgtgctatgcacggtgcgttgtcagcgttttgcgcttgcatgcgctacacgcgtcgtccaacgcggagggattcttccacattaccatgtgtcactccccctatggagggttccaccccgcccacacggaaataggttaaccatacctatagtacgggtgagcgggtcctcctagggcccccccggcaggtcgagggagctgaaattcgtgaatccgtgagtacacggaaatcgcggcttgaacgtcatacgtgaccttcggagccgaaatttgggcgtgccccacgaaggaaggcgggggcggtgttgggccgccgccccctttatcccacggtctgataggatgcttgcgagggcacctgccggtctcgtagaccataggac

SEQ ID NO: 576 - DNA, 445 bp, Hepacivirus B
accacaaacactccagtttgttacactccgctaggaatgctcctggagcaccccccctagcagggcgtgggggatttcccctgcccgtctgcagaagggtggagccaaccaccttagtatgtaggcggcgggactcatgacgctcgcgtgatgacaagcgccaagcttgacttggatggccctgatgggcgttcatgggttcggtggtggtggcgctttaggcagcctccacgcccaccacctcccagatagagcggcggcactgtagggaagaccggggaccggtcactaccaaggacgcagacctctttttgagtatcacgcctccggaagtagttgggcaagcccacctatatgtgttgggatggttggggttagccatccataccgtactgcctgatagggtccttgcgaggggatctgggagtctcgtagaccgtagcac

SEQ ID NO: 577 - DNA, 379 bp, Hepacivirus I
cagggtttcgaccctggcccggatacctatcgccttacgccgaaaggtaacgagtaggagtcgggtccccaggcccttaccgccaccaagccaggtggggaggtatgggagccggggggtgcagctggtagctccatgggggacgccccgtgagcggatgctgcatcgataccgggttagctctctgggagagcggcacttgacaccacgaatccgggaaccggacaatcgccggcgtgggacgcgttgcctccgtggccgagcaatttggcatgcccgtggtgaagagtgatggtgggggggggccccccttccagtaccgtactgcctgatagggtcttgcctcaagcccagagagtcgaggctgaaaaccgccatc

SEQ ID NO: 578 - DNA, 557 bp, Hepacivirus J
gccgctcccgaaagggagtccggcgcgtcatcccactccgaggagtggggtggcgtccccgtgtgccggggaaccatgaagcctaagggcatccacattttagaatgaacttgaagcttcgtttcgctggccggaaagtcctgggttcccatggccagggttccgcaggtgggtaaatcccggtggggttccatccaggatatacggcaggcgggcgtagtccggcggttcggacgacgtgtgggtcgcctacggtggattgttcacaggatgggcactccggtgtgaataggccccgtcagggtgcgctgacgttaaactcaggccttgcctggtgttcggggaggattgcagggccacgccgcctctaagggccgtatggcacagtacttcttcgggcgggttgtcaaggccctccaacgcgacaccagtgcctcggcaggcatggggccacccagctcggcgtcccgcacacagacggcgtaggggaaaatcagcaatgtgaccccgggtggcattttccttctctctacttccatgcatgatcaaccgcaatc

SEQ ID NO: 579 - DNA, 280 bp, Hepacivirus K; identical to SEQ ID NO: 49

SEQ ID NO: 580 - DNA, 675 bp, Unknown
  Description of Unknown Icavirus sequence
cgaagtttaagctaagcaccctcgggcgttcccggattatgtgatcacatcaatttgatggctggtcaccacgcaacgcctggagagatactcttacttttctcttaagatccccggtcatttgacgcttgtaggatgatagggttattttccactataaatactttcatactcttggatgttctatatccaagacgggaggacctaccccgtaccccttagaggtgagatgccaagaacaggccctttctgttctctcgacaatggcatcataggcaacaagcatcacaccaagattgctaagttttgttaagagttcttcaagctatagggtggctgtagcgaccttctgatgcctgcggatttccccacggagcgatccgtgccacaggggccaaaagccacggctaacgcccatcaggagcggcacttaccccgtgccccacccttgaaacttgaatgttcacactggcttctctcggctttctgaactgtctgcttgttggggccccgaaggatgccctggaggtaccccattttatgggatctgaccaggggacacctcagctctctaagttgctggtgtttaaaaaacgtctaagggcccccaccccttaggtggagggatccacctttcctttattttttaaaactcttttatggtcacaattgttt

SEQ ID NO: 581 - DNA, 113 bp, Unknown
  Description of Unknown Antarctic penguin virus A sequence
ctaggagactacgcagtgggataagatgactatgatgtcgtacgggcagaagccagtacagtcgaagtcgagaccgacgtcgaggatttgactctgcctgacctagtgccatc

SEQ ID NO: 582 - DNA, 214 bp, Unknown
  Description of Unknown Forest pouched giant rat arterivirus sequence
tattggatccgcctccgggcaaaggttactttcttgtacctcggcttagccacagggtgaccccttgtacgtaggggccccgacgtagcactggtctgacaacaccttctcggcatttcaccttctgcccgctcttccgggcggtggtgtcaagaagcagcagtgctcttctcttttcttcctgcagttcaccgagccctacggggggtaggtg

SEQ ID NO: 583 - DNA, 561 bp, Avisivirus sp.
cattcccctttccccagccatgggttaaatggcccctcaccaggttcggtgctgtctaggcttccagtaaagaagtcaaccgagcattgaacaaaacctcagtgggtatggtagttaaccccgtccactggacaactttgtcctcttaaaagtggatcaatccaccccaactccccccctagccacctgagccatggtggatagcagtgacgaaactagggaccccaatacctctagtgccaagagaattcccccctcgcgagaggtgctcttgggcccgaaaggctagttggcagggtgaagtgaaggaagctgctagcgtggcaaccttaagcgtagcccgaagctgaccttagaggttaaccctagtggaccactggatgaagctgtggaggtggtggataggaaagttggccacttgtgagtagatgcccagaaggcataaggctgatctggggccagtgactataccgttccggtaaacctggtataaaaaccatgaaagcaagtgggtttaaaatttcttctaattccttcatttcagtagtgataactggcaga

SEQ ID NO: 584 - DNA, 134 bp, Unknown
  Description of Unknown Avian paramyxovirus penguin sequence
accaaacaaggactagataacccacgtgaccgttaactggaaaataagatgttgtaggggcgacctagttggaattcgaccccggctccgaaacctctaattgtggttattggcagtctagtctacttctaacg

SEQ ID NO: 585 - DNA, 121 bp, Unknown
  Description of Unknown Newcastle disease virus sequence
accaaacagagaatctgtgaggtacgataaaaggcgaagaagcaatcgagatcgtacgggtagaaggtgtgaaccccgagcgcgaggccgaagctcgaacctgagggaaccttctaccgat

SEQ ID NO: 586 - DNA, 302 bp, Bat Hp-betacoronavirus
ttaagcttcggcttgttgcataggaccggaaaggtactatctaccctaactcttgtagttagactctctaaacgaactttaaaactggttgtgtccttcagtagtctgtatggccattggaggcacaccggtaattatcaaatactaagaagattcatagtacatccttgtctagcttttggttggcagtgagcctacggtttcgtccgtgtcgctcacaattatccacacagtaggtttcgtccgctgtggttgagttgctagtccgttgctgtttcgtcagccatctacaactcgacacc

SEQ ID NO: 587 - DNA, 458 bp, Unknown
  Description of Unknown Basella alba endornavirus sequence
ggaatatggctaatcggcttattctaatcaaacgcaaaagacttatgacacagaccggacctgaacgaggtgataaaacacctcgttcaggttcaaaacgtagaagattcattcctccgattagaaatacaactacgtctaagcacgatagagatggtatcaagatcggttttagaccacgagaaaatcggcaaatgaaagtacaagttgggtggtttaaattaccaagaacagtaacattcaagaacaacggcaacccgtttgttacctcatttcgtaaattgtttagaagtaacaaagataaattatttaatggtgggaagaacctaagtacagtaccagccagaagtagtgaaatgacagaaatgtttatgttcatgtccacgctagagggccaattgtcaatccaagatcgagatccaaaaataatcaataagtctatatacatgatagaggta

SEQ ID NO: 588 - DNA, 675 bp, Ball python nidovirus
ccccttcacccataggcactaggagaacaggataacccctaacggggcatcctgcctgtgacctttcagattcgctagttagatatcttcacagactctgctaggcttctgacccagtccgttcccaaagtccgttaccgcccgagtagcgcttaggcgcgaaagggacggaagtacctccagtaagcgaaagctgaagtaagggaaatacggcaagactaacttgttagtcttacagtgtggataacctggtagttatccccgacaagagacctgactcgatgtgaaaacatccaactaggttggcttcaaactagctacaggcaggatatcccccaacggggcctccaaggaatcgagaaccaaccctcattccacgtctgtagtaagcaaaaacaggggcgatcttcaccgacacctctcaccacagagcacaccaacctctgtgaagccaatttcctcgtccaaggacaggttattgagggtcaactttcttccgaccagaagaagggatttcctaccaaaagaaaaaccaaatccaccaacaccacaaggtaaaacaacaacttgtgaagccaatacttagtcaaagactaactattgagggtcaactttctcttcaatagagaagggatttcctggtaaaacaaataacaacaactaacatcagcaact

SEQ ID NO: 589 - DNA, 150 bp, Sapelovirus sp.
gacaggtgttttggagggcggatgacgatatctggctggccaccagggaataacggcaaatgtctgatcatacggttcacaagtctaccggcgatagtggttcaacaccatgtgtagcagggattcttgcgtatgtgaaggcgacagtgc

SEQ ID NO: 590 - DNA, 496 bp, Unknown
  Description of Unknown Bat Picornavirus 3 sequence
taagcggaaagcattcttgtcccccggtcagtaacctataggctgttcccacggctgaaagggtgaacatccgttacccgcctcagtacttcgagaaacctagtacgcctgatgattccaaattggtatgatccggtcaaccccagaccagaaactgtggatgggggtcaccattcctagtatggcaacatacaggtgtccccgcgtgtgtcacaggcccttacgggtgccatttcggatgagtctggccgaagagtctattgagctactgttgatacctccggccccctgaatgcggctaatctcaaccccggagccactgggtggtgaaccaaccacttggtggtcgtaatgagcaattctgggacggaaccgactactttggggtgtccgtgtttcttttgttcatattaaactgttttatggtcacaacacaacttggtacgatttgtgattattcactgctcacttgtcacagtaaatatacacaatcatc

SEQ ID NO: 591 - DNA, 419 bp, Unknown
  Description of Unknown Bat Picornavirus 2 sequence
gggttttacgaaacccgtatacaccagaccttttctcccctccccctccacctaccttttccccctctttggaccgaaacaaggacacgtaagtggaaacgcgattttatatgtggttggccaccacggaataacggcaattgtctacatgtgggaagtgcaacctccctagccgataacccctgaccgggtgtgtaggataggaaaggtgcccactgtgggcgacaggttatggtagagtggatacctagccaggggcaatgggactgctttgcatatccctaatgaagtattgagatttctctgctcattacccggtgatggttgtgtggggggggccccatacactagatccatactgcctgatagggtcgcggctggccgaccataacctgtatagtcagttgaattcagccaag

SEQ ID NO: 592 - DNA, 410 bp, Unknown
  Description of Unknown Bat Picornavirus 1 sequence
gaaacccgtatacaccggaccttttctcccctccctctccacttacctttttcccctcttcggcatgaaacaaggattattcaagtggaaacgcgatttaatatgcggctggccaccgcggaataacggcaattgtgtatctgctggaagccaagcctgcctagccgatagcccttgaccgggtgtgtaggatagcccaggaaccagcaatacgcgacaggttatggtagagtagatacctagccaggggcaatgggactgcattgcatatccctaatgaaccattgagatttctctggtcattacccggtgatggttactagaggggggcctctagtactagatctatactgcctgatagggtcgcggctggccgaccatgacctgtatagtcagttgatttgagcaat

SEQ ID NO: 593 - DNA, 675 bp, Iflavirus sp.
acgaatcggtatacgcttcggtacctattgttgcaagttcgttccctattttcgatttgcctgcccgaatttgactcaaacaattgtgacatactatgtctctgtttgaaagcactacacgagtttgcgcccagcatgtatgttttcaagtcttttgtataagtctgcctctatagtggttttctttgaccttaaagccttgtcaaccatcctatatgctgcatcgagacttgatgtcaatctgcctctactacgcaaatgtctagtaattagttataaggttttactattttccctcatttccaattttagtttgtagtgtatgtgagtatcattcttactccgactgttaagagaaaccaatttatagtcgttaaatatgataaatggaatgaatgatggtgtcattttaaaaacactcttctctataggcgtaagcattctcgctcttagagtcgtaaagaagaaatgccgtgtctatcagtatgttatgcgatttattttctgccacgcgcttctagtgcaatctagttgacatacagacattgcctaccactcgcgagggtcgaccggtagtgtaaggagtaagtgatgatttccgcttattctgtaccctttgcctggtgaggacagatcctgactaattttaaatataaatgaacactagcttccaag

SEQ ID NO: 594 - DNA, 210 bp, Unknown
  Description of Unknown Bat dicibavirus sequence
gtatagcaccggaatggtattttactactccaagtatacgtactaggagttaaaccctgtaatttacaggggatttagtgacttttatccgtaaaagtcgattggacgttaatcggtaacgaggccaagtaccgtgaaccaatttaaaaacgtattttctcatgtggtagaaccaacttggaaatagcatggcatataggttgtttaggg

SEQ ID NO: 595 - DNA, 212 bp, Betacoronavirus sp.
gataaagtgtgaatcgcttccgtagcatcgcaccctcgatctcttgttagatctaatctaatctaaactttataaaaacactaggtccctgctagcctatgcctgagggtttaggcgttgcatactagtgtcttaggaatttgactgataacacttccctgctaacggcgtgttgcactctcagtctaagcctcccacccataggaggtatc

SEQ ID NO: 596 - DNA, 277 bp, Betacoronavirus sp.
atttaagtgaatagcttggctatctcacttcccctcgttctcttgcagaactttgattttaacgaacttaaataaaagccctgttgtttagcgtattgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatc

SEQ ID NO: 597 - DNA, 675 bp, Cardiovirus sp.
tacgatcgctgtacattccactactgccaattagctcccccttcccgttgctcccctctataaggagagccttctcttgcaaaggtgaagccttcacccccggtcgaagccgcttggaataagacagggttattttctcctctcctcggcgcttgcctcttctaagctgaataggttctatctattcaggcggatggtctggtccgttccttcttggacagagtgtgtatctgggttttccggatctcgaccacacactcaccagagctcaggagtgattaagtcaaggcccgatctgcggcgaaaaggaaatgaagtattttgcagctgtagcgacctctcaaggccagcggatttccccacctggtgacaggtgcctctggggccaaaagccacgtgttaatagcacccttgagagcggtggtaccccaccaccctgcaaattatggatttgacttagtaactaaaagattgacttggcatacctcaacctgagcggcggctaaggatgccctgaaggtacccgtgttgaaatcgcttcggcgaccatggatctgatcaggggccctgcctggagtggttctatcccacacagcgtagggttaaaaaacgtctaaccgccccacaaagaccccggcagggatgccggtttcctttttaccaattcttgacact

SEQ ID NO: 598 - DNA, 675 bp, Unknown
  Description of Unknown Breda virus sequence
atcacctagtacttacaagcgggtcaaaccgccctccggaacggtcataaccccctcccgaacgtgcgcttgacgtgactggtctttcagtctagctttctgagaaatactccggggttgtaacccaccattttgacctttggtcagtttggtaacactccaaccaaacagcatctgacccacctccagcttgctgcaggccatttggaccaaacgggttcagatatcttgtggctaaacctctgacccacctccagtttactgcaggccttttggactaaacgggtacagactctttgtggttagtttttaactacccactgtttagccgccaacctgatttttattgttacaaaattttgtgtttacacattattttcttacggttggcagtttgtttggttgtttgcacagtttttgctgataccaatttttactgtgcttttggtgttttcggctaaggctgtttttcacatacttagtttgcttgaagtaaccttcacaacatctgttttgtttttggtttctaaggtaaagagtttcaggaaaaaacataggcgcccatcttgtggtgtctagttttaattaatctggcaaacaagtatcaagtcatcgactccctttggagtgagacttacgagtaccaattcgcctattttggccatccatataaaa

SEQ ID NO: 599 - DNA, 383 bp, Unknown
  Description of Unknown Bovine viral diarrhea virus 3 sequence
gtatacgcccagttagttcaggtggacgtgtacgattgggtatcccaaattaataatttggtttagggactaaatcccctggcgaaggccgaaacaggttaaccatacctttagtaggacgagcataatgggggactagtggtggcagtgagctccctggatcaccgaagccccgagtacggggtagtcgtcaatggttcgacgcatcaaggaatgcctcgagatgccatgtggacgagggcgtgcccacggtgtatcttaacccaggcgggggccgcttgggtgaaatagggttgttatacaagcctttgggagtacagcctgatagggtgttgcagagacctgctacaccactagtataaaaactctgctgtacatggcac

SEQ ID NO: 600 - DNA, 537 bp, Bovine rhinitis A virus
ttttgcggctctgccgccgttcgggttttacctgttttcacagagcaaaacaggacctctagtttcgtgcttaaacgagatcatgctcgaactagaactataacgctggtcactggacccgtgccgcgccttgcggatctttgcgggaatggtggctagtgggctgtggaagtgactctaaccacacgcccctcaagtgtgggaaaacacgaactggtgtagcgacgacgataggccttgggacaccctctccagtgatggagacccaaggggccaaaagccacgccttgtgccctgtcgttcacaaccccagtgcagttcgtgccagtacctgcttttgggaagtgtgctttggacagctgaaaacagtcctagtgggagactaaggatgcccaggaggtacccggaggtaacaagtgacactctggatctgacttggggagagcgggtctgctttacagacgccactctttaaaaaacttctatgtctcgtcaggcaccggaggccgggccttttcctttaaaacaatacacttt

SEQ ID NO: 601 - DNA, 248 bp, Unknown
  Description of Unknown Bovine picornavirus isolate TCH6 sequence
ttggatctgagcaggggccccctttgggttgctttacaactcaactgggggttaaaaaacgtctaacccgacacgccagagggatctggtttccttttatttcttttactcaccactggatgcagattgacgataaacgttgttgtttgtgactattgacttgatctgcttctacgggtttactttcactgttatacttcttgctttgtttggtgttcactgtactttgtctccttctacatttcaca

SEQ ID NO: 602 - DNA, 486 bp, Bovine nidovirus
caccaatagattagtcaagctgtctataggcataaactaacccccaaccccattaccccggggccaggtgggccgccgccttcgggcaaacccgtgcgctggtataatcaaggttcacagccagattcactgccggttagctagtggggcggtagcctggcaaaacccgaagaggttggaaagggaacttcagggtagtttatcctaggctagcgtagctacagttcggtcaagataaccgtcctggtgctagggctagtagagacagtggtaacttggacaagggtccagggccactttagggaataccctacggaaggctaggtccgtaaggaagacccccgcagttgtccgcggttgagcagagctcctgcgtagacaaaaggcaaaaagtggattacattcgcctgcaggaaaaggcaaacgtcgttggagtcggagctaaagtactggacgattgataccacgcctgctgcggtagataaa

SEQ ID NO: 603 - DNA, 293 bp, Hepacivirus sp.
ccatcgacactccaggctcacggattaagttaggttccgccgaagcgggctaaccaggcccctagtaggaggcgcctatcccgtgagccctttccccacggattgagtggagctggagctgggaaggaccgagtacggtccaatcgagaagaaccctgatgaacattccaggcctcttcggtagatttggatatatccaccagtgaaggcggggtcgtgggtacaggccccctagtccacacagcctgatagggtcctgccgcaggatccgtgggtgcggctgtacatgtacc

SEQ ID NO: 604 - DNA, 472 bp, Mitovirus sp.
gccccgccgaccttcctatttatttctaaataggaaggtcccgactagtcggataattcggtttaacgaattatggttagatctattaaagttaaaatagattgaatttctttctcatcctccttattcctatactttgggagtaaatgacaaatgtctatcctcaaaccgaaatggcttaagtgatgaatttgaaagaaaggtaggttttaagaatataaggcatcaatatattatacccttgaatgttaagtgaccacggcgtgacgattagggctatcttaggatagacagccatctaacgcgacagcagtggaaatcagcttagcatctcaagatcatgtataatatatacataaccttacaattataaaaccaaaccaaaacacactataattttatataaattatagaagtatcggacctggacggtacctactattaactgatagtagccaaatgcaggaagctc

SEQ ID NO: 605 - DNA, 315 bp, Mitovirus sp.
ggaacttttcagttccagaagtggctttattaagccttcaaagtttacactttgaacctgcgattaattccccatagtgactcttgttactgtgaattaggaatagttgtagttcaacttctaatgaggtgaacaatataataactcatcttattaaccctatacgtagacaattgtccaaagagacagttggaattctgccaatctggaatgtttggtacgcgtagaagataataagagaccctctattcccctgcctcatgactaagtcatggcccggggtgtaatagagatacttttatatattatacaatc

SEQ ID NO: 606 - DNA, 675 bp, Unknown
  Description of Unknown Canine picodicistrovirus strain 209 sequence
cgctctttatacaaatctgtcaaccctttgtataactctaagccgaacaattatagctaggctttttattatataacattaattaggcattagcgttgtcgccaatctcttggtaatcctaaggatacctttcctgttgactaagatgaagcgccttcggttaccgatgcccggtgtccacgaagccatcgtggtcggccgcgtcccccacctctcccaacttggactccatgttttcagtaggtgtaatgattagtattattgattctgctcgttcaatgtgtttatcttcacgatctgggacccaacacatgcttcactcatgtttaaatgttggttccctcattttgaagacacccaaaccatagagtgcgagaatgaggatttctacttccattctggtaacagaaatgaattcctgcgtgtgtctcgtaaatggaatctttaagaacttcagataaatcgaacaatacactaatacaagttgttttctaccaacatgttcaatgcggctaatctgaccgtggagctgtgaagcgctcaaacccgagtgttgtatacagtcgtaatgcgtaagtccatgaggaaccgactactgttacctctgttggtgtgtttctcctttcctctcttttattattatttgttattgcaaatactacaactttgatcaac

SEQ ID NO: 607 - DNA, 107 bp, Canine morbillivirus
accagacaaagttggctaaggatagttaaattattgaatattttattaaaaacttagggtcaatgatcctaccttaaagaacaaggctagggttcagacctaccaat

SEQ ID NO: 608 - DNA, 601 bp, Kobuvirus sp.
tttaagtgttgtgcccaatctcttgactcctgctggaaccaccgaccagtagtgtccaaaatgccaggtggaaaatcctcccttcccctctgggcttcatgcccggcatcctccccccagcctgacgtgccacaggctgtgcaaagaccccgcgaaagctgccaaaagtggcaattgtgggtcccccctttgtcaaggcgtcgagtctttctcccttaaggctagtcctgtcagtgaactctgtcgggcaactagtgacgccactgcatgcctccgacctcggccgcggagtgctgccccccaagtcatgcccctgaccacaagttgtgctgtctggcaaacattgtctgtgagaatgttccgctgtggctgccaagcctggtaacaggctgccccagtgtgcgtaattctcatccagacttcggtctggcaacttgctgttaagacatggcgtaaggggcgtgtgccaacgccctggaacgagtgtccactctaataccccgaggaatgctacgcaggtacccctggctcgccagggatctgagcgtaggctaattgtctaagggtattttcatttcccaccctcttcttcttgttcata

SEQ ID NO: 609 - DNA, 291 bp, Alphacoronavirus sp.
cttaagtgtcttatctatctatagatagaaaagtcgctttttagactttgtgtctactcttctcaactaaacgaaatttttgctacggccggcatctctgatgctggagtcgtggcgtaattgaaatttcatttgggttgcaacagtttggaaataagtgctgtgcgtcctagtctaagggttctgtgttctgtcacgggattccattctacaaacgccttactcgaggttctgtctcgtgtttgtgtggaagcaaagttctgtctttgtggaaaccagtaactgttccta

SEQ ID NO: 610 - DNA, 623 bp, Cripavirus sp.
gcaaaatcggtagtacgttaaacgtacgaccaccgatgagactgaaatgacactagttgagattatttcaatatcctagtgttataaagtcaatatttgttggttgatcgtttcgtcaatcgatggcgctgacagccggaaagacggcaataataaaaaccaagatttagtttttaagttttgattgaattgcaaaagctatcttgaatagacaatcaaaatattaagtaaagcaaaagcttcttaaagaagacaatatttaattagttagtaaccaaacctcatcgtgcccctaagggttaaccggttacgtaaaagcgtagaggtattaaggtcactgcggagacctaaaatccgcaattttatgttttgtaatgttttagttatagacttagatgtaactataagagtttataaatacttgtttcaagatttatagacaagatctgatcctatggattttagataaccttcatgttagtggatagtgtgtgtacctatctaaacgcataaggctcttatttcatatttaaagtaggactatgtattacggcgcatctaacggtaacgttagtcaagaccggagaatctcggaatgaattttagtaattcccaaatttata

SEQ ID NO: 611 - DNA, 675 bp, Unknown
  Description of Unknown Human coxsackievirus A2 sequence
acctttgtgcgcctgttttatatccccaccccgagtaaacgttagaagttacgcaaccccgatcaatagtaggtgtagcactccagctgcatcgagatcaagcacttctgtctccccggaccgagtatcaatagactgctaacgcggttgaaggagaaaacgttcgttacccggccaattacttcgagaagcccagtagtgccgtgaaagttgcggagtgtttcgctcagcacttcccccgtgtagatcaggctgatgagtcaccgcgatccccacaggtgactgtggcggtggctgcgttggcggcctgcctatggggcaacccataggacgctctaatacagacatggtgcgaagagcctattgagctaattggtagtcctccggcccctgaatgcggctaatcctaactgcggagcacatgccctcaaaccagggggtggtgtgtcgtaacgggtaactctgcagcggaaccgactactttgggtgtccgtgtttctttttattcttataatggctgcttatggtgacaattaaagaattgttaccatatagctattggattggccatccggtgactaacaaatcgctcatataccagtttgttggttttgttcccttatcacatacagctcataacaccctcttatatttactacaattgaatagcaagaa

SEQ ID NO: 612 - DNA, 436 bp, Unknown
  Description of Unknown Coronavirus AcCoV-JC34 sequence
agaaacaagtagtgttttaaaaaccttcaaattagtgcctgtaacatctttgcaatgaaagtagcgctcactagcctctatgcaaagaatgttaaaagaaatacgaagcatttaaagaatacaatctatctaggataggtacaattctcctccccctcttgacttcggtcaactcaactcaactaaacgaaatcccccttgcatggttccgacccgtgtaaggttgtgtatttcgtgcagtcgttgcccttactagtgtaagcgtaacggcatctaggtttgcacgtcttggaggaaacggtgtgtacgtttctagtgtttacgccgtatcggttccggcccgataggtattgcattagacgtcctgggtggttctgcctgcccttgtgtgattcggctgttccgtcagtttggtcacctcacacgtccttaagac

SEQ ID NO: 613 - DNA, 457 bp, Unknown
  Description of Unknown Chicken picornavirus 3 sequence
gggtatggtggttaaccccgtccactgggcatctttgccctctcagaagtggatcaatccaccccaactccccccctggttacctgagccacagtggactccggtgacgaagctagggaccccaatacctcaagtgccaagagagtccccccctcgcgagaggtgctcttgggcccaaaaggctagttggcagagtgaagtgaaggaagctgctaacgtggtgaccttaagcgtaattcgaagctgacctttgaggttaaccctagtggaccactggaggaatctgtggaggtggtggttaggaaagttggccacttgtgagtagatgcccagaaggcataaggctgatctggggccagtgactataccgttccggtaaacctggtataaaaaccatgaaagcaagtgggtgaaattttctctttttatccttcattcagcagttgatattggcaaa

SEQ ID NO: 614 - DNA, 675 bp, Unknown
  Description of Unknown Chicken picornavirus 1 sequence
gtggccgacttgcagaacttcctaccgaaccaccacctcacccccataactccttccctctacaccttccgctatggtggttaccactgctttattgccttgactgagaatggccaccccctcgacacctgccccctactgccccaccgcgcaaccttttgcttgtccactcggttggagaggcatgggggccccgttcatatccccagtccagttggtgaccttccccctccccgtccggtagatggtccagagggctttgccgacgccctctatgatgcttgcttgtctttcctccgtcagcgcgagcatgcacttgtcgagcccacggaaacacagcctagcttttgcttctctcaccctgcgtaccctgggcgccttccgctcgagattcgctttgttcgacaccctggcgtccccccaccgctacgtgattttactcgtggcatacaccgccctggcgttcagtacattccactgcctaatttgggtggcctccctcaatctcccgcaccccccattgcgcacgtcatcaccgccgccgctaacgcgatccggcgcggttctcactggcactgtcccctcgtccgccgggtttccactcatggttggcttttcacttattctggttactggtgttccatcctacttcatgatggtcgccatgaccatgac

SEQ ID NO: 615 - DNA, 675 bp, Orivirus sp.
aaaccctcacgagtgcttgtggtaggtcccaggccaatattcttcgtaaggcttggttccaattttccaccactcgtgtttgggttctggcctatggtacccagaggggcggtttgggggaattaactccccctcccctgtggtcctataccaccccacacctctgtgggctttctttactatcttcttgttttccgacttttaaacactaggcaggcgcgcctagtcatacaccgcccggctggtctttccagctcttgtgggcggtgcgcgctggtccatcgtgcccagcgacatagcaccttgtggacacctccgaacgccctcccctgtatggggtggtgcccaggggtttcagtgtggtgacacactccctggggcccgaaaggctagtgtgcaacaggtgaggtacagccagctgcccccgtggctggagggaccaagcttgtgaagcacacctcaccttcttgggggtgggctagtaagtggtgaaagcatagtgtccgtgtcgctggccaacactttgggtcaagtccagccactcagtgagtagatgcccaggaggtacccctagtggatctgacttggggcctgttacttaatgcaggttaaaaactatgaaagctgagtagtgtagcccggctggtggcttctcttccttattcattctatttt

SEQ ID NO: 616 - DNA, 651 bp, Gallivirus sp.
ggttaacttgtttaaccaaggcttccgtgcagggcttgcacgttaggagttcatgttgattcatgctccaatgcccaaaactttgtgtgtttgtttatgtctttcccaaagtttcccccaaggtttcggtactcaaaccttaattcctagtcccatcttttgggccttagtatctaggaaatgtacccgtgccttgacgaacgtaagaaagctgtcttttattgaacggttctaatgaactaagtataactggctcgcgccacctggtgtgtgccgttggaattcccccatggtaacatggtccaacgggcccgaaaggctagtgggcaatcggtcctccaaggaaggggttcccaccccgacctgaacaggatttgatgaagctcacctcccaggctcctaaccccaaggaagtttacttatagtaattagaatttagtatgtaattgctggcaatcttgctagtagtcaggaacgttatgaccaaatgagtagacccccagaaggtaccccattatatgggatctgatctgggcctcatactgtgtgtctccccacatatgaggttaaaaccatgaaagtttggtccaaaatattcttttccttttatcttttctttagtggtgacgccattatatcagcagtttgctg

SEQ ID NO: 617 - DNA, 303 bp, Unknown
  Description of Unknown Chicken calicivirus sequence
ggtgcatcatcactgaacaccctcgggcagagatgcaagggtggaagtcactcctgccccctggcaacatgcaggtgcccgatcccaagcttagttctgacttcctctcctgggtggtgcaacactccaaggttgatgaacaaacctggagggacctctgggcaacctggtctctcgaggatctccggcgcatctccacagactacctcatgctcccggaacctgagaagaacactgatgcctatgatggctggctgatggtcggcgagatgttgacctttgccggtctcattggctgggagg

SEQ ID NO: 618 - DNA, 524 bp, Unknown
  Description of Unknown Carp picornavirus 1 sequence
agctacaggaaagagagagataatcacagcacataaatacaactacagaagagagcctttccctgagcactatttacagcaaaccacgctgggaaaagtggtagcatgacccacttacgggttacttagtataggattttaatatcttcgattctttattttcttttcttaactaagttcgcccggactttatccgtgttttattttagtttaagcaaaattgagtaactaagtttttaacctgccaaatggtgagaagtaactctgtgaaaataccatttgtgcatgaaattgtcttgaaaactcttaggcttttggggggtcccactgctgtttggaggactattgacagactctattgtagttgagtagtgactaatgataacgatttgcgtattacgcaatgggctgtacccgttagatttagtatgccggggggaggggtcccactggattgcactatgtaacctgacagggcgtctgccgacgcactacaatgaggataagatcggctgtttttattt

SEQ ID NO: 619 - DNA, 675 bp, Unknown
  Description of Unknown Falcon picornavirus sequence
cgcttggaataagttgagaggaattatgcatgctagttgtgtttgtttacaactaattgttctaatccaagtgaagctcttcgcttggggcggcacgacacttgccgtaattcttctaccgtccctccacaccttgtggatgaagggccggatgtgtggcctctggctaacccctctctctggggtgatgctactggatgttttactcctagaccaaatcacatgaactcctcttgatccacttcggtggggctatgagcctgcggattaatagctggcgacagctaccccaggggccaaaagccacggtgttagcagcaccctcatagtctgatgcccaagggctgatgttgggagctagtagtgtgtgtctggcctatgtttaggacttctggccaagcgcagaggagtggggctgaaggatgcccagaaggtacccgtaggtaaccttaagagactatggatctgatctggggccccctcacgtggctttaccacgtgttgggggttaaaaaacgtctaggccccaccagcccacgggagtgggctttcccttaaaaagcccaacaatatttatggtgacaattcactgtttcttctttgcaatttttgtattcttggactccttattttttgtttgcttgattttagtggacattcagattcaaatacaaa

SEQ ID NO: 620 - DNA, 675 bp, Unknown
  Description of Unknown Equine rhinitis B virus 1 sequence
cgacaggcacaggtcgctccgagttctagtagtgtgggaacttgttactactgatgaaacgaggtagtgacactcagtacctgcgaacgaggtcggggccctcccttcttccttcacccaactttcacttttcgttccactttagcaggggtcttctttctatccccctggcggcattggaactagccgtcgcgtcttaacgcgcagccctgaaggccccacaccttgtggatcttgccgtgggtatgtttctggcatgtgtttctcaagcctgcaaccgaagccgaacagccacatgaacagtttgagcgtggtagcgctgtgtgagttggcggtggatccccctcgtggtaacacgagcccccgtggccaaaagcccagtgtttacagcacctctcacatccaggacgaccccatcctggcgctcactcttagtagtatggcttagtacgcattaggtggtaagccgagctctccctcggccttgttctgaatgcacacatgtctaggggctaaggatgtcctacaggtacccgcacgtaaccttcagagagtgcggatctgagtaggagaccgtggtgcactgctttacagatgcagcccggtttaaaaagcgtctatgcccctacagggtagcggtgggccgcgccctttccttttaaaactacttgttct

SEQ ID NO: 621 - DNA, 675 bp, Equine rhinitis A virus
aagggttactgctcgtaatgagagcacatgacattttgccaagatttcctggcaattgtcacgggagagaggagcccgttctcgggcacttttctctcaaacaatgttggcgcgcctcggcgcgccccccctttttcagccccctgtcattgactggtcgaaggcgctcgcaataagactggtcgttgcttggcttttctattgtttcaggctttagcgcgcccttgcgcggcgggccgtcaagcccgtgtgctgtacagcaccaggtaaccggacagcggcttgctggattttcccggtgccattgctctggatggtgtcaccaagctggcagatgcggagtgaaccttacgaagcgacacacctgtggtagcgctgcccagaagggagcggagctcccccgccgcgaggcggtcctctctggccaaaagcccagcgttaatagcgccttctgggatgcaggaaccccacctgccaggtgtgaagtggactaagtggatctccaatttggcctgttctgaactacaccatctactgctgtgaagaatgtcctgaaggcaagctggttacagccctgatcaggagccccgctcgtgactctcgatcgacgcggggtcaaaaactgtctaagcagcagcagaaacgcgggagcgtttctttttcctcatttgtttc

SEQ ID NO: 622 - DNA, 224 bp, Unknown
  Description of Unknown Equine arteritis virus sequence
gctcgaagtgtgtatggtgccatatacggctcaccaccatatacactgcaagaattactattcttgtgggcccctctcggtaaatcctagagggctttcctctcgttattgcgagattcgtcgttagataacggcaagttccctttcttactatcctattttcatcttgtggcttgacgggtcactgccatcgtcgtcgatctctatcaactacccttgcgact

SEQ ID NO: 623 - DNA, 675 bp, Enterovirus sp.
actctggtatcacggtacctttgcacgcctattttataccccttccccatcgtaacttagaagcaacaaacaaactgcccaatagcagcacaacacccagttgtgttaggggcaagcacttctgtttccccggaagggtctgacggtatgctgtacccacggcagaagtatgacctaccgttaaccggccatgtacttcgagaagcctagtaccattatgaaggttgattgatgttacgctccccagcaaccccagctggtagttctggtcgatgagtctcggcattccccacgggcgaccgtggccgaggctgcgttggcggccagcctacaccatacggtgtaggacgtcaagatactgacatggtgtgaagagcctattgagctacgtggtagtcctccggcccctgaatgcggctaatcctaactccggagcatccgccagtaagcccactggaagggtgtcgtaatgcgaaagtctggagcggaaccgactactttgggtgtccgtgtttcctgttttacttattgtttggctgcttatggtgacaacttatagttatcatcataagctacttggtcttgccaaccggagaattatttggttatttgttggtttcataaacctacagtcgtattttcctgtcttattaattgttctcaaaattaacaaca

SEQ ID NO: 624 - DNA, 675 bp, Enterovirus sp.
taccgctgcaccagtgagctggtacgctagtaccttcgcacggagtagatggcatcccccaccccgtaacttagaagcaaagtacacatctggccaatagtggcgctgcatccagccgcgcaacggtcaagcacttctgtttccccggtccgcaagggtcgttatccgcccagtccactacggaaagcctactaaccattgaagctatcgagaggttgcgctcggccacgaccccggtggtagctctgagtgatggggctcgcaaacacccccgtggtaacacggatgcttgcccgcgcgtgcactcgggttcagcctattggttgttcacctcaacatagtgtaaatggccaagagcctactgtgctggattggttttcctccggagccgtgaatgctgctaatcccaacctccgagcgtgtgcgcacaatccagtgttgctacgtcgtaacgcgtaagttggaggcggaacagactactttcggtactccgtgtttcttttgttttattttgaattttatggtgacaattgctgagatttgcgaattagcgactctaccgctgaacattgccctgtactacctaatcgcatttcacaaaacctcagagataccaagctcttacattgatctgcttgttttcctgaatctcaaatataaattggaacaagcaaa

SEQ ID NO: 625 - DNA, 107 bp, Morbillivirus sp.
accagacaaagctggctaggggtagaataacagataatgataaattatcatacttaggattaatgatcctatcaattggcacaggatttggataaaggttcacagtc

SEQ ID NO: 626 - DNA, 348 bp, Unknown
  Description of Unknown Dianke virus sequence
tgttttcaaccataatactactactacaagtataaaaccccgtccgtctgtcggagacgctaaactctgaccaccaatctagccacatcagttgcttaaagaacctcttgagacactctcccacttaacatcttttaggaatcttcgatgctacaacaacttggctagtgaacaataaatccgtacaattcacagttgtaagaggccataggtccagactttgaaaggtttgtttctattgttacaaatacttagattaacagaggctatttaatagtgctcatcacgttaacagagtaaccttgtgcaatagtatgagcttgttgtaaaacgtcttgatacgacacc

SEQ ID NO: 627 - DNA, 270 bp, Hepacivirus sp.
cactcaatactacactccgcatttggggagaagcgctggcgttcgcggaaccgcgttaaccatacgcgtagtacgagtgcgacagaccccggtgctactggtggtagcgagacacgagccgaagtctgtggggggaactccacttagagggcatgcccgggcgtaggcttctgagttgggatgggccccaacttggcccctgagtgggggggtgttacgacctgatagggtgcgggctggcgcctaccactttccagtcgtacatgagtc

SEQ ID NO: 628 - DNA, 415 bp, Narnavirus sp.
gcccggggggtgcagtcctgtgaaagggtctgcaccatactatatatgtatatgattacatcccaaaaggcgacttcgttcaggttttaaatctgacgtaggtccagtaaataagcatgtcaaaacatgtaagtttatcctgtaatctactctcataagatgagataagatgatattgcagttcccatgtaaataaatccattatgaattcattcatataaggtagaagtggtaactatggttgaaacattaatataaaacggtcattttgcatgaacgtcattaaggaactggcataccaatgtctatttagtgactatgatatttagagtatcccttatattaattaacaattattccttttagcatatcatccgacaacaaattttaaaagaagaaatattactcattaaaa

SEQ ID NO: 629 - DNA, 675 bp, Torovirus sp.
gtacttacaagcgggttaaaccgccctccggaacggttacaaccccctcccgaacgtgcgcttgacgtgactggttctcagtctggctttctgagaaatactccagggttgtatcccaccatcttgacctctggtcattttggtaacaccataaccaaacaaactctacacacctaacccacctccagcttgctgcaggccttttggactaaacgggtttaggtgttttgtgaccaactcgtctacccacctccagttttactgcaggcctttttggactaaacggctttagacttttgtggttagtttttaactacccactgtttagccgccaacctgattttcattgttgtaaaattttgtgtttatacactattttcatacggttggcagtttgtttggttgtttgcacagtttttgttgataccaatttttactgtgcttttagtgtattctgctaaggctgtattttacatacttagtttggttgaagcaatttttacaacatttatattgtttttgatttctaaggtaaagagtcttaggaaacaccatagacgccattcttgtggtgtctagttccaactaatctggcaaacaagtaccaagtcattgactcactttggagtgagacttacgagtaccaatttgcctatttcggacatccatataaag

SEQ ID NO: 630 - DNA, 675 bp, Foot-and-mouth disease virus
acaagcttgacaccgcctgtcccggcgttaaagggaagtaaccacaagcttacaaccgcctaccccggtgttaatgggatgtaaccacaagatacaccttcacccggaagtaaaacggcaaattcacacagttttgcccgtttttcatgagaaacgggacgtctgcgcacgaaacgcgccgtcgcttgaggaggacttgtacaaacacgatctaaacaggtttccccaactgacatacaccgtgcaatttgaaactccgcctggtctttccaggtctagaggggtaacactttgtactgtgcttgactccacgctcggtccactggcgagtgttagtaacagcactggtgcttcgtagcggagcatggtggccgtgggaactcctccttggtaacaaggacccacggggccgaaagccacgtcctgacggacccaccatgtgtgcaaccccagcacggcaacttttctgtgaaactcactctaaggtgacactgatactggtattcaagtactggtgacaggctaaggatgcccttcaggtaccccgaggtaacacgcgacactcgggatctgagaaggggactggggcttctgtaaaagcgcccagtttaaaaagcttctatgcctggataggtgaccggaggccggcgcctttccattataactactgacttt

SEQ ID NO: 631 - DNA, 310 bp, Unknown
  Description of Unknown Feline infectious peritonitis virus sequence
acttttaaagtaaagtgagtgtagcgtggctataactcttcttttactttaactagccttgtgctagatttgtcttcggacaccaactcgaactaaacgaaatatttgtctctctatgaaaccatagaagacaagcgttgattatttcaccagtttggcaatcactcctaggaacggggttgagagaacggcgcaccagggttccgtccctgtttggtaagtcgtctagtattagctgcggcggttccgcccgtcgtagttgggtagaccgggttccgtcctgtgatctccctcgccggccgccaggaga

SEQ ID NO: 632 - DNA, 143 bp, Unknown
  Description of Unknown Farmington virus sequence
acgacgcataagcagagaaacataagagactatgttcatagtcaccctgtattcattattgacttttatgacctattattagacccttcacgggtaaatccttctccttgcagttctcgccaagtacctccaaagtcagaacg

SEQ ID NO: 633 - DNA, 528 bp, Unknown
  Description of Unknown Avian infectious bronchitis virus sequence
acttaagatagatattaatatatatctattacactagccttgcgctagatttttaacttaacaaaacggacttaaatacctacagctggtcctcataggtgttccattgcagtgcactttagtgccctggatggcacctggccacctgtcaggtttttgttattaaaatcttattgttgctggtatcactgcttgttttgccgtgtctcactttatacatctgttgcttgggctacctagtgtccagcgtcctacgggcgtcgtggctggttcgagtgcgaggaacctctggttcatctagcggtaggcgggtgtgtggaagtagcacttcagacgtaccggttctgttgtgtgaaatacggggtcacctccccccacatacctctaagggcttttgagcctagcgttgggctacgttctcgcataaggtcggctatacgacgtttgtagggggtagtgccaaacaacccctgaggtgacaggttctggtggtgtttagtgagcagacatacaatagacagtgacaac

SEQ ID NO: 634 - DNA, 626 bp, Rhinovirus sp.
ttaaaactgggtgtgggttgttcccacccacaccacccaatgggtgttgtactctgttattccggtaactttgtacgccagtttttccctcccctccccatccttttacgtaacttagaagttttaaatacaagaccaatagtaggcaactctccaggttgtctaaggtcaagcacttctgtttccccggttgatgttgatatgctccaacagggcaaaaacaacagataccgttatccgcaaagtgcctacacagagcttagtaggattctgaaagatctttggttggtcgttcagctgcatacccagcagtagaccttgcagatgaggctggacattccccactggtaacagtggtccagcctgcgtggctgcctgcgcacctctcatgaggtgtgaagccaaagatcggacagggtgtgaagagccgcgtgtgctcactttgagtcctccggcccctgaatgcggctaaccttaaacctgcagccatggctcataagccaatgagtttatggtcgtaacgagtaattgcgggatgggaccgactactttgggtgtccgtgtttcactttttcctttattaattgcttatggtgacaatatatatattgatatatattggcatc

SEQ ID NO: 635 - DNA, 675 bp, Unknown
  Description of Unknown EV22 sequence
ccttataacccgacttgctgagcttctataggaaaaaaccctttcccagccttggggtggctggtcaataaaaacccccatagtaaccaacacctaagacaatttgatcaaccctatgcctggtccccactattcgaaggcaacttgcaataagaagagtggaacaaggatgcttaaagcatagtgtaaatgatcttttctaacctgtattatgtacagggtggcagatggcgtgccataaatctattagtgggataccacgcttgtggaccttatgcccacacagccatcctctagtaagtttgtaaaatgtctggtgagatgtgggaacttattggaaacaacaatttgcttaatagcatcctagtgccagcggaacaacatctggtaacagatgcctctggggccaaaagccaaggtttgacagacccattaggattggtttcaaaacctgaattgttgtggaagatattcagtacctatcaatctggtagtggtgcaaacactagttgtaaggcccacgaaggatgcccagaaggtacccgcaggtaacaagagacactgtggatctgatctggggccaactacctctatcaggtgagttagttaaaaaacgtctagtgggccaaacccaggggggatccctggtttccttttattgttaatattgacatt

SEQ ID NO: 636 - DNA, 675 bp, Cardiovirus sp.
tccgacgtggttggaattaacatcttttccgacgaaagtgctattatgcctccccgattgtgtgatgctttctgccctgctgggcggagcgtcctcgggttgagaaaccttgaatcttttcctttggagccttggctcccccggtctaagccgcttggaatatgacagggttattttccaaactctttatttctactttcatgggttctatccatgaaaagggtatgtgttgccccttccttctttggagaatctgcgcggcggtctttccgtctctcaacaggcgtggatgcaacatgccggaaacggtgaagaaaacagttttctgtggaaatttagagtggacatcgaaacagctgtagcgacctcacagtagcagcggattcccctcttggcgacaagagcctctgcggccaaaagccccgtggataagatccactgctgtgagcggtgcaaccccagcaccctggttcgatggccattctctatggaaccagaaaatggttttctcaagccctccggtagagaagccaagaatgtcctgaaggtaccccgcgcgcgggatctgatcaggagaccaattggcagtgctttacgctgccactttggtttaaaaactgtcacagcttctccaaaccaagtggtcttggttttccaattttgttgactgacaat

SEQ ID NO: 637 - DNA, 292 bp, Human coronavirus 229E
acttaagtaccttatctatctacagatagaaaagttgctttttagactttgtgtctacttttctcaactaaacgaaatttttgctatggccggcatctttgatgctggagtcgtagtgtaattgaaatttcatttgggttgcaacagtttggaagcaagtgctgtgtgtcctagtctaagggtttcgtgttccgtcacgagattccattctacaaacgccttactcgaggttccgtctcgtgtttgtgtggaagcaaagttctgtctttgtggaaaccagtaactgttccta

SEQ ID NO: 638 - DNA, 282 bp, Unknown
  Description of Unknown Hubei zhaovirus-like virus 1 sequence
gtgcaggatggcctttcccatcttaagtggtttgtaggatttcgtgggtccataccccccgatttcttggtacgtattccatgcacggagaatacgaccaaaactcttatttcaaaaaatattattttttactcttgtgggctgagtgcgacccaccagttccagcttagcaacctggaagttgttggagatttatggaaccaaattacacatgcgtggagtgccgccactccgtatctgttcactcattacgcgattaagttctgcgacgagacgagcgaa

SEQ ID NO: 639 - DNA, 600 bp, Unknown
  Description of Unknown Hubei tombus-like virus 9 sequence
ggaccatccaggcaggtgtaggctagtaccctcacctgacctgtcgcgatgtttggctttgtgaggcttgtgggaggatcccttggcccatgcattgctgctgtcatcgtgaaaaatgttgtatgctcgcacctggcgtggaggaaacggcatttgacggatgctaaggaggatttgaaggtcctcaactcccatgccttattacaagtcccctttccttcaagcttcgagcccacgtctgtgacgagcagaggtgaggttggagtcaagaggagtcctattcaagctgttcgcagcaagaaccataaaactttcattgctcaatgggcaagagcggctaaggctcggttctcgtttgcacgacagtgtgagcggagccctgtaaatgtcgcggcccttcatcggtggtttgcatcggagtttaagaaaattggcatgaacttgctccaggtttcgtacgtgatcgatgaggttgttgaacttagtttggaaccaacgtatgagcgtatggtgtccgaaactaagaagcaattccgtcatcgggcacgtatggaatactacaacgagaaaaaatgccttgaaaagatccactaggaacgc

SEQ ID NO: 640 - DNA, 251 bp, Unknown
  Description of Unknown Hubei tombus-like virus 32 sequence
ttcgggtttacccgcgtaagcggccacactgactggttgtcggtgttgaatttgtataccagatgaggagacgttaccaccgtctcggcagtgctacgtctgggaaaggactgtgatagtggacagtcctaccgtctttagatacattgcgttgtgtatagcccgggagggattaactaatagcaacgcaatgcacacggcggttcggatttgcttgactgatggaaagacatctaagttctaattgaacc

SEQ ID NO: 641 - DNA, 341 bp, Unknown
  Description of Unknown Hubei sobemo-like virus 3 sequence
gagatgtttgcgtgggccgttgcgctgcgggcggcccacctccctacggggaccgtgagacaccgctggggaaggcccccacccccggccaaggggatcctgccgagaggcaggagaaagaggcccagccctctggggcgcctttaggggtgcctgggagggaagtacccgagccgggcggccggtcgggtgcggctgtgcagttcgaggctaaccgtaaggaaggcctgagctgcctcggcttgtcggaaaggaagacgaaggcacttatcaaggctttcaggaagcaggagcgcaattacagcgcgcgccgtgcggcctggattaaccgcatttggccg

SEQ ID NO: 642 - DNA, 675 bp, Unknown
  Description of Unknown Hubei picorna-like virus 2 sequence
agcaacttctttctgaaaactagctagagttcgacgatctctctggctaatgacaaataaccaatcaaaaagtcaaatgttcatgtatatatatatttagtagtgacctttatttagaaaaactttagatgatttatcgtcaagttgccctttgtgaagcgatcagctttttatatcgtttcatttttgtatacgtcttaattgacgttgtaagtacgttttgcatacctcacattgaggatagtatcgtttcctgactaagaagttaaactagtctaccaatagcaaccatataggatatagctttgtttttaacaaggtttaatctgatcttatgctcttgcttacggtgttttatgtatagaaaagtttttataaaaactacataattgtcaaaagaaaaagcgttacgtactgacgcatttatgttcacagtgtgacacaaaccttctatattttgattgtaaaataggctttgcctgaccatttatcaaatacaaactgtttcaaacgcctctccgagccatattggccgacgcgaatcgacataacagggtgagtttacagctgcagttgcagccgaggatccacttatttgagagagaattactcttaacgaaattttgaagtcacttctacacagttaagtctgagtaggcgttatccaaacgtaaa

SEQ ID NO: 643 - DNA, 174 bp, Hepacivirus sp.
acatgggggggggctgacagtgagtacactgtgccaagcaggtgctacgctatgcctaggtgctgctgtaggccaaggacatgtcccagtcatcccaggtgagggggggggttcccctcaccgctgccactgcctgatagggtcctgccggagggtctcggtgtccggctgtac

SEQ ID NO: 644 - DNA, 675 bp, Unknown
  Description of Unknown Harrier picornavirus 1 sequence
gatgtgtgacggtgtaattactttccggatcccactttcctattataactctttcatcccaaggttagggaaagaacctggctcggtaccaccagaccctccgccacgctagtggactctccggagataacggtaccccctttgtagtcacctgtgctggtgaagaaccacctagtattgcttgggtgcgtgccgcctagcttccatttcttctggagcactgtgcaatgaggtttccccacttggtaacaagtgcctcaggtcccgcaaggatactgtggggtggtgtgaccgcagggagctgtctccacggctcctctaatgttacgccgctatccacaggccagtgcgtgtcatcgatcccggatgacagagctagtattgcgaacccccaagtaagaaaagtggctagtaacctgatagctggtgaagagggtgggtcagttgagtagatgccctagaggtacccgaaaggatctgactagggacccgtgactatacattaggtaaaccgggtataaaaaccatgaaaaactgaccacttttcttttaacctcttctttctttttatgtgtgttaagtgttttgagtttggactgtaccttgcccgcctttcttggattttctctatcgctcttcttacacctactgttatcaaggcactctttagagata

SEQ ID NO: 645 - DNA, 500 bp, Kunsagivirus sp.
tttcaaatcggactccggtagttataccggagcccggtttggacgcttgggccgcgttaacagccccccacccctttcccactgactgatttctcggattggactcatttgcattgctaactctgattctggatttccccgtttatgtcgtcgcggtcggaagtgcacgtacttcgacgttgatctgatggcgtttgtttccaggggggaggtggcggcagaaacgcccccgccgtaaacttcggcgggccacgcctgtcaagccactccctggggccgagcgcctgaggtgatacagagagataagcacactgggcgctgacaacgcccgggacctcagtgagaagagcagtagggccgtgtttatgggactccattggatatcccccgcttgtcggaactcacggctactccgggttgggaagcccgcgactggtactgtactgggtgatagcctggtgccttccctctcactgttgtatgaaggctgaaaaccccct

SEQ ID NO: 646 - DNA, 675 bp, Kobuvirus sp.
tatagtttgcctgtttctcgcaccgttaccgctcgttcgggaatgtgaaactggcacccctcctctcccctaccaccctttctccttcgcccccattcataatttacaacgccgcacacagcggcggccgccaagggctagcctggcggttataaaaggaacctgggtctttccctcttcaagccaaaaggtaggttccctgtgtccctgaatgctcggtgaggaatgctgcaccgtaacgctttgtgaagtgtttgcaagttctggcccggcaagcctacagagtgctgtgatccgctgcggacgccatcctggtaacaggacccccagtgtgcgcaacagtatgttcagacttcggtttgttcacttgctttcatggaccattgcgcgaaagtgcgtgcgccatatccctgtacttcaggtgtgcttctctggaccctaggaatgctgcgaaggtaccccgtttcggcgggatctgatcgcaggctaattgtctatgggttcagtttcctttttctttactccacaattgactgcttaactgactctggatcttgtgcttccactgctctttctgctctcaaaacggcttcacttaccaactctcacctttcgaccaacaccatttacacactaacttttttcgactcttctgactcctggcttggtgaagac

SEQ ID NO: 647 - DNA, 608 bp, Kashmir bee virus
tacgtacaattttgacgcttcgttcatgcaacaatgaactcacatgtggcgctcggtagtaaccagaggggcgtcattcccccgtatggagtggagaatataagctaccgactcgagctgtagaataattcagcaacttataacgaacacgaattttagtcgacgaaaccattttagcctttaattctatgatttgattataatgataaacagctcatgtaactgtctaaactacataaatacaactggattacgaacctttaagtaactatcttagatgaagtctagtagtctcccaatatttccgtgaaaagaatgagggacgagatagctctatttaaagacgtgaggctttaaattctgataaatacattacctgagaattcctcctttaggagttagaatttgaaaagattagtacctatacttaatagaatttaaatatgttaataatgctgaagacaagtatttcgattattaacctctatatttctatataaagtatctgtgagtctcagtggacatcacagtaaggtcgcagttaacttgtaatcttttcattcctgtgtcggagcagtggtaatggagccggacgatttcgccaaaac

SEQ ID NO: 648 - DNA, 332 bp, Unknown
  Description of Unknown Jingmen picorna-like virus sequence
tgtgtttttgtcaagataattgttctgtgattaacagtgattgtggttcgtgtaatgcgacgcagtcaaatgctagttttgatgaagtgtatgagagagtggaaaacttatctcataagaagattgaagagtgtgtagatcaggctattgatcgagcttctaagcttcgtgattacaagcttaatgttcacaatggctcccgacgggaatcatctgatcctctctttatttcgccccattcgttgttatcgcttggggtatctaagtttgttgcgtttgagcagcatcacagtttcgcttcagttgagtctctgaagttgcttgctctgtct

SEQ ID NO: 649 - DNA, 145 bp, Mumps orthorubulavirus
accaaggggaaaatgaagatgggatgttggtagaacaaatagtgtaagaaacagtaagcccggaagtggtgttttgcgatttcgaggccgggctcgatcctcacctttcattgtcgataggggacattttgacactacctggaaa

SEQ ID NO: 650 - DNA, 138 bp, Mosavirus sp.
gaagttgatcatgaacttggttattggtggaacgcacatgaactcccaacaatgatcttgaagacacagcgtggtaacaattaccatgcccttgtggctgcccaagacattgatggctattgggtgatttatgatgac

SEQ ID NO: 651 - DNA, 675 bp, Unknown
  Description of Unknown Miniopterus schreibersii picornavirus 1 sequence
gttgtcgacccgttgcttggtttaagcatgaggtggattcccccgattatgtctacccgttactatggcgggcggtcgtttcagggtgtttctactgaggactgcaccaagtctttatcttttattctcagatctccggctgtttgacgcttgtaggacagcaggactattttctcttaatctttttctacccactagggtcctatcctagtggagggagggtgccaccccttctctctttagagagtgcgcctggcggtctttccgtctctggaaaaaggagcacatggcatgctacaattggcacaagaaaacaagctttgcggattctttctttgtactagaggaagctgtagcgaccctgtatggcgagcggactcccctctcggcgacgagagcctctcgggccaaaagccaagtgttaatagcacccatacaggcggcagtaccccactgccctttctcaacatacaatgactgatgaaccactgttggtttttctgacacctttgtagtaggattccaaggaatgtcctgaaggtaccctgttagcttacgcgcaggatctgatcaggagtctctttacagtgctgtacactgtgcaagggattaaaaattgtttgaggaatccccgagatagtggtctatctcttcctattttgttttacagacacg

SEQ ID NO: 652 - DNA, 381 bp, Unknown
  Description of Unknown Linda virus sequence
gtatagcagcagtagctcaaggctgctatacgattggacataccaaattccaattggtgttagggaccacctaggtgaaggccgacgacaggtagccattcctgttagtaggacgaaccgttatggtggactggttgctcaggtgagcaggctgcaatgcgtaagtggtgagtacaccacagccgtcaaaggtgccactggtaaggatcacccactggcgatgccttgtggacgggggcgtgcccaacgcaatgttagcggtggcgggggctgccatcgtgaaagctaggtcttgatggaccttgttgcctgtacagtctgataggatgccggcggatgccctgtgacagccagtataaagaatatccgttgtgattgcac

SEQ ID NO: 653 - DNA, 675 bp, Unknown
  Description of Unknown Lesavirus 2 sequence
tctttctttattttcttatgtaactcttctttttaagttttattttgcctacttgtgagcttatgcgggaccactgtcttagacaaccccacatttgtcatgagtaagtacacgcaaccattacgattactttttaaccgtctgaccttttgataacaactgaagttaggcgtgaaacatgcatttataccaaagtagccccgcatttccccactacggtgggggggctaccctactggctttggaactgtagccattatgtgttgcctggctttcaggatctcacaacacaacagttctctcacaatggaatatgggtgagattgcagtgacatgaacaagtatctagtagtacatagactcaagcctagttgcctgcggaacaacatgtggtaacacatgccccagggtccaaaagacaagggttaacagccccttctaggtgtctgtgtgtgaagaatactttagtagtgttgttatgatctcacctgttagtacagaatgagtatggcttggtgaaggatgtcctacaggtacccattatatggatctgagtaggagaccactagtggtggctttaccgccaggtgagtggtttaaaaagcgtctagccaagccaacagcactagggatagtgctttctatattttatattttcagtgtatatggtgacaa

SEQ ID NO: 654 - DNA, 675 bp, Unknown
  Description of Unknown Lesavirus 1 sequence
gtaactaataagcaagttttactgcctgcaaactgcttcaatgggaccaccgcttcggcgacccctttgttgagtttgtatgtttttaagtaatctttgcaaccatacgattattttagccgcctctctataatgatcttgttatagtgggacgtgaaacattggtttttctcacacacgtccggtcacccgggcgtgtgttcttccgtaagtcctatccacataccatcgtgggtaggccagcatgtttgcacaggctgtgacagtgtgggtgggctttccacctctcaacaacacactgaattgcaatgcactcacggaggaaatgacaatttggttatagttttgaactgtgctagtaatttctttcacattaagccatgttgcctgcggaatcacatgtggtaacacatgcctcagggcccaaaaggcacgggttagcagccccttcatggtgtgttagaagtgaaaacacatagtatgagctataatatctttgttgtcttctctgtagtgtaccccgccaaatgtaaggatgcccagcaggtacccattttatggatctgagctggggattgatagtgtatctataaatgcactgatcaatttaaaaagcgtctaagtttggcacaaacactggggacagtgtttttcctttattcttttatttgatta

SEQ ID NO: 655 - DNA, 638 bp, Unknown; identical to SEQ ID NO: 30
  Description of Unknown Phopivirus strain NewEngland sequence

SEQ ID NO: 656 - DNA, 377 bp, Pestivirus sp.
gtatacgagattagctcatactcgtgtacaaattggacgtagcaaatttaaaaattcggatagggtccccatccagcgacggccgaacggggttaaccatacctctagtaggactagcagacggatggactagccacagtggtgagctccctgggaggtctaagttctgagtacagaacagtcgtcagtagttcaacgctggtaaaccccagccttgagatgctacgtggacgagggcatgcccaagacacaccttaacctggacgggggtcgtccaggtgaaagtacccatctttgggtgctgggagtacagcctgatagggcgctgcagaggcccactgacaggctagtataaaaatctctgctgtacatggaac

SEQ ID NO: 657 - DNA, 494 bp, Unknown
  Description of Unknown Quail picornavirus QPV1 sequence
tttgcatcagttcgcccctcccctcaccatacctttttcccctctttaggactgatacttggttatgatgagcagaggatttcgcaagttatgcttcttgataaaaagtaattcacgaatcatgggattatagcctggaagtgaacactcatgtggcaagtgggttagtagctctccatgttcccatgtgcagtggactgacaacagtgagttcggggttgtgtagtaaagggaaagtattacttacccgcacctgctatacgtggtgtacgtaggatacgagttagtagtgcttagcaactttaaactggtgctgaaatattgcaaggtcactgaagttgtgaacgcgaacgctccgccactgccatgtatagcgtgcaatgcataaatggtgcactacatgatacgagggaatgggaaaccctccatggccgaatgcagggtgacagcctgccggcggatgcctgttgttagtataatccgttgtttgccac

SEQ ID NO: 658 - DNA, 443 bp, Sapelovirus sp.
acactcatttcccccctccacccttaaggtggttgtatcccctacaccctaccctcccttccacataggacgaataaacggacttgagattaaggcaagtacataaggtatggtttttggatacacttaaatggcagtagcgtggcgagctatggaaaaatcgcaattgtcgatagccatgttagcgacgcgcttcggcgtgctcctttggtgattcggcgactggttacaggagagtagacagtgagctatgggcaaacccctacagtattacttaggggaatgtgcaattgagacttgacgagcgtctctttgagatgtggcgcatgctcttggcattaccatagtgagcttccaggttgggaaacctggactgggtctatactgcctgatagggtcgcggctggccgcctgtaactagtatagtcagttgaaaacccccc

SEQ ID NO: 659 - DNA, 189 bp, Unknown
  Description of Unknown Porcine reproductive and respiratory syndrome virus 2 sequence
atgacgtataggtgttggctctatgccttggcatttgtattgtcaggagctgtgaccattggcacagcccaaaacttgctgcacagaaacacccttctgtgatagcctccttcaggggagcttagggtttgtccctagcaccttgcttccggagttgcactgctttacggtctctccacccctttaacc

SEQ ID NO: 660 - DNA, 675 bp, Enterovirus sp.
gaaccttagaagtttacacaaacaaagaccaataggagtccaacacccagttggattgcggtcaagcacttctgtttccccggacctagtagtgataggctgtacccacggccgaagatgaacccgtccgttatccggctacctacttcgggaagcctagtaacattctgaagtctctgaggcgtttcgctcagcacgaccccggtgtagatcgggctgatgggtctccgcataccccacgggcgaccgtggcggaggccgcgttggcggcccgcctatggcgaaagccataggacgcctcttagatgacagggtgtgaagagcctactgagctgggtagtagtcctccggcccctgaatgcggctaatcctaaccacggagcgtccaccagcaatccagctggcagggcgtcgtaacgggcaactctgtggcggaaccgactactttgggtgtccgtgtttccttttgatcctattttggctgcttatggtgacaacgataagttgttatcataaagctcttgggttggccacctggaaaaagttatcagtgtttgatattgttcggctctcacgcctaccaataagacaagccctatatttacttgttgcattttactcgtcagaagaaatcacagagtatcatttggatttgttactcacattaaggacaag

SEQ ID NO: 661 - DNA, 591 bp, Unknown
  Description of Unknown Pigeon picornavirus B sequence
tttatttagctgttaagttttatttgtgccgagaccccatagtaggatcttggtgttccacattaagctctcccgaccacacatccaaacgataggcggtgtaagggctccctggctaagtgtttactcattgctagggaagtgttgcgacccgttaccagtaggaatacaggaggtcttagttgcctaaccagataaagtggtgctgaaatattgcaagctcaatgtctggcgaacgacggactaccgttgaactattgttaacgcccgcgtgtgtaggcaacacacgggttagtaggtcacttacattgacatccgtgccgggaaagcggatctgagctatcgattgcctgatagggtgccggcgggcgcggtacgtgtggtatagtccgctgtcttggggtatggcgtctactcggttttgttgtcgttttggaatgtcccatgtcggagatgtcgttaccggtgtttgctttacttgtgcacgaataagaaaacagagtttggagttttggaagttaatggataacatcaagtttccattgcgttcctcgtgcattttaggccagaaaatcgaccacaaaatcgaga

SEQ ID NO: 662 - DNA, 620 bp, Unknown
  Description of Unknown Picornavirus HK21 sequence
aaacgggaggatcggctttggcttatcctcttaatagtctacaaaactgggctgactggtgggggagctactagatccgggagaggactagttaccccgcgtaacttccctttttgtcactccctccacctaccccttccctgtcccttagactcttaagtaggtgtgcaggcctggccccccggaaatgggcaaatcggacgtttgcgtgtagggtcgttctgaaaggtggggcccactccaccgtagtaggatcttcctgtttcacgtttaaacctctccgggcaggtatcgctagacactaggctgtataggatggcacgcacttaggtttcgcaccttcctagtgcgccaagatcccgcttttgagctcaagtacatggttctttgtaagatgtctaaccagaggaggtggtgctgaaatattgcaagccactctggcgaacgtccactgcattgcctggaacaggctctctagcgccctccactggtagcgcggtggtgggttagtaggatacctatatggacaggggatgcgggaatacccctcactagctagtgactggttgatcgactggcggcggatccagtgatacttgcataatccgcagacttgggag

SEQ ID NO: 663 - DNA, 675 bp, Unknown
  Description of Unknown Picornavirales Tottori-HG1 sequence
agccatgaactagtgtgcgattcccacagtgttggtcgaaagccccgcactgtctactcgcatatttgactccagtccttccgcagccagcggttaggttctggttaaagtgcattatgtgcacggcgccacgcagaactgccagaaatggtaagctgcgcccaacgccaacggtttgtgtatcccgttagtcacacgtttacagctgttcgttaacagtagggttttgtcgacccggaccccttaatgcgcgcgaattcaaccgcgcctagtcggcaatggtatcatttaatcccatgcactacgggagaaatttgagaccaaagaattcctgagggccactgcttgctctaagtgcaatgcctcgggagacttctgtcaggagcctagcggctttcaaccgcgacagctaactcctgcgggatgtttggtgtccatacttactggcgtcctcacaacgctaagtggatgttgtccacaggtaggcaaacaccgagccccacattcaggagacctgtatgaacgatcctatcagcattagagttggaattgggtgtgctaacgtccgcataagtgcaccccgtggtaacgctgggaaactatccagcgcaacgtactgtcctcaatgtctagggaaggaccgccctaagcgtacaaccgggccatgtgtcgagc

SEQ ID NO: 664 - DNA, 549 bp, Hepatovirus sp.
ttcaaagaggggtccgggattttcctggtccccctctttgggcacccttggctcgggggtgtgaataccgtgctcgcgtttgccgtgcgttaacggcttcatttatgtttgtttgtctgttttattatgttggtttgtctgtttgttatgttggtattgttcgtgtttaatgttatgaccacattacactccagccaatgaagaacagatggtgcggttattgctggcggaattcctaacgtcctggatccgttggtacgcatcacaaaacaatttgcagagagagtggtgaaacggcttgggaatccctgagtacagggaaatcacactgatagctcatcttggctgttttcagtcatggaccttatgcagtgtaatttgggtgtaccccccatagcttaggaggaatgttctgtcttggcactagagtgggacgctgatgcctccgtgtctaggatggtctaagggacagaatggggtgcctctgatgccatactacctgatagggtgctctcacggcctctgcatcttagtgagaagttcaatttt

SEQ ID NO: 665 - DNA, 107 bp, Unknown
  Description of Unknown Rinderpest virus sequence
accaaacaaagttgggtaaggatcggtctatcaatgattatgatttagcacacttaggattcaagatcctatcgactggagcaggcttaaggtaaaggttctttaaa

SEQ ID NO: 666 - DNA, 675 bp, Rabovirus A
ctacggatatttgcatgacccgctttctatcgccccaacaatcccctttgtaaccacaagctttactcaggctagcagcccgactagctgtttggaagaaaaggctagggcacacaccaacaacaccgaccccactggtcgaaggccgcttggcaataagactggtggaacagggtcgcctgtagttgtttggaacattctttctaatgactttgtcagcggtgctactcacaccgtaactcttctaccctatccccacgcttgtggaactaggaggggatgagtgattcaagtaagtactgtcagaatggtgaaaatgatctgattctgaaacgctatggatccatcgaaagatggggctacacgcctgcggaacaacacatggtaacatgtgccccaggggccgaaagccacggtgataggatcacccgtgtagtttgagatcatatcaatgttcatagtctagtaagatgatttgaaatctaactgagctgatggctaactgcttgtcttattgcggcctaaggatgtcctgcaggtacctttagataaccttaagagactattgatctgagcaggagccaaagtggtctttcccagctttggttaaaaaacgtctaagccgcggcagggggcgggaggccccctttcctcccaaaacttaatattgattgt

SEQ ID NO: 667 - DNA, 410 bp, Shingleback nidovirus 1
ctgtgagtaccgacaggctcgaagtctattatgaggcgtcgaaacagaaaacctgtaacaactccggtttcatctatcactgccgtcaagaggcagaagaggacgaccacgtgtcaccagatcacttgtatctgtttcagtcaggaagtcaacttttcgacgaagttcgaccattcatcgacccgctgaaaagcgtagaagtcgatgaagatgagctccaaagagccatcgccgatttcgacaaccaaagtgactgtttccactccttcgagctcgtgaatttcgagctgaaacaacaaatcaacgagaacgagtggtacggttattataattacgacaaccaaaactgcaaagttcagttgccagtcacatgtcgaatcgaggacgtaacctgggatcaggtttacgtg

SEQ ID NO: 668 - DNA, 666 bp, Unknown
  Description of Unknown Seneca valley virus sequence
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaat

SEQ ID NO: 669 - DNA, 675 bp, Unknown
  Description of Unknown Sclerotinia sclerotiorum dsRNA mycovirus-L sequence
ttgaattaatcttttacgtttacgcgcataaaatcaggacacatctcttgtatactttagtatatcaatgatgtttttgttttatgcgattaatcgtaagagaacttctttccatccgcctgtatgggcgggataataagttcaccgccttggtcgaggcgcaaacttgtatgtgcaaaggtgagctatatgctcgaaatagtcgtaactaacacacagccactacctgtagagctctattgatccggaatcctttagtgggaatgcagagctcacaccggacctgcgggtatcttcggcgttagggacttctgtttcagccttgaatcatttacctttataccttctctgaggcgcctgggccgggcgcgatattaagtacaagtcaaggacatcgcgggtagtggtctaatcagccgctagtcctgctggagagttccaacttagttgggtgtggtgcatactagctggatagagtaggtatgtattgctaacgtatgccggaggctatccgtcctcggtagaacgtgccgaggagtagtctctgcagacccccgaacgcgtggggtctttacttaaatgtaggcggagggagcgctcgtaggtggaacgactgcctcccagtcgaatgcaagattttgcacgcggaccagtctgcccggcaattcccgggtg

SEQ ID NO: 670 - DNA, 675 bp, Enterovirus sp.
ctccggcacagccgcaccagtgcactggtacgctagtaccttttcacggggtagtcggtatccccccccgtaacttagaagcatgtaacaaaccgaccaataggtgcgcggcagccagctgcgttgcggtcaagcacttctgtctccccggtccgcaaggatcgttacccgcccactccactacgaggagcctagtaactggccaagtgattgcggagttgcgttcagccacaaccccagtggtagctctggaagatggggctcgcacatcccccgtggtaacacggttgcttgcccgcgtgtgcttccgggttcagtctccgactgttcacttcaacatcacgcaaccagccaagagccgattgtgctggagtggtcttcctccggggccgtgaatgctgctaatcctaacctccgagcgtgtgcgcacaatccagtgttgctacgtcgtaacgcgtaagttggaggcggaacagactactttcggcaccccgtgtttcctttattttattcttattttatggtgacaattgcagagatttgtgatattgcgactttaccgttaaacatagcactgcattacctggttgcattccacaaaacttcagagattcctagttcctacattgacctacttgtttatttgaatcttaaatacaaacttgagcaagtgaa

SEQ ID NO: 671 - DNA, 245 bp, Unknown
  Description of Unknown Wobbly possum disease virus sequence
cggctgtgagtgcttagcatatgctagagtactacagccgggtgttggagtcatatgcactggttgcctgtataatagtcgggatctgtctgacctacattatctttgggagttgcttatcacgacaattctcgaagtgtctgtcgacagcttacccccgattcgacaaggccccttgtccaccgcagacctatcgattttcaacgagaacactatcagaggtttaaatttaaaactcaccaaca

SEQ ID NO: 672 - DNA, 631 bp, Avian orthoreovirus
gctttttcaatcccttgtgtcgatgttcccgtatgtcatctggttcatgtaacggtgcaacttctatttttggtaacgttcactgtcaggcagcgcaaaactcggcgggtggtgatcttcaagcgacttcctctcttgttgcttattggccctacttggctgcgggcggtggtctgctcgttgttattattataattgttggcgctgtttgttgttgcaaggctaaggttaaagcggacgcagcccgaaacgttttctaccgagagctgttcgcacttaattcgggtaaaagtgatgcaggacctccgatttaccaggtttagtgtacgacgatttgagttttcacctttcgtcttagaggagtgcactactccatctttcacgactataactaataccgatccggctctctactttaacattgagtttccgtcaagtcatcgtctctcccccttcattccagaactgttgtctcagccttgtaccgttcacgtttcattgattcggagattcgctctctgtgcaaccttatctagtatttgtgaatacgactgtgcgctactgccatccatcaacgctattacgacgatccctacaccaggtgcgtcatcatctctgattgttcattggg

SEQ ID NO: 673 - DNA, 665 bp, Kobuvirus sp.
ggggcctcggccccctcaccctcttttccggtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 674 - DNA, 655 bp, Kobuvirus sp.
ccccctcaccctcttttccggtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 675 - DNA, 645 bp, Kobuvirus sp.
ctcttttccggtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 676 - DNA, 635 bp, Kobuvirus sp.; identical to SEQ ID NO: 352

SEQ ID NO: 677 - DNA, 625 bp, Kobuvirus sp.
ccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 678 - DNA, 675 bp, Unknown
  Description of Unknown Picornavirales sp. isolate RtMruf-PicoV sequence
tttgctcagcgtaacttctccgggttacgtggagaccaaaaggctacggagactcgggctacggccctggagcacctaggtgctcctaaagacgttagaagttgtacaaactcgcccaatagggccccccaaccaggggggtagcgggcaagcacttctgtttccccggtatgatctcataggctgtacccacggctgaaagagagattatcgttacccgcctcactacttcgagaagcccagtaatggttcatgaagttgatctcgttgacccggtgtttcccccacaccagaaacctgtgatgggggtggtcatcccggtcatggcgacatgacggacctccccgcgccggcacagggcctcttcggaggacgagtgacatggattcaaccgtgaagagcctattgagctagtgttgattcctccgcccccgtgaatgcggctaatcccaactccggagcaggcgggcccaaaccagggtctggcctgtcgtaacgcgaaagtctggagcggaaccgactactttcgggaaggcgtgtttccttttgttccttttatcaagttttatggtgacaactcctggtagacgttttattgcgtttattgagagatttccaacaattgaacagactagaaccacttgttttatcaaaccctcacagaataagataaca

SEQ ID NO: 679 - DNA, 675 bp, Unknown
  Description of Unknown Apodemus agrarius picornavirus strain Longquan-Aa118 sequence
ttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattaca

SEQ ID NO: 680 - DNA, 675 bp, Unknown
  Description of Unknown Niviventer confucianus picornavirus sequence
ccctttcataaccccccccttttaacccaacccttcgtaaccgtacgcttcactcgcctttgggtatagcggcccaatgtgctgaagaaaaggatacgctataaggggccaacgggtggtggcccttaagaccacccaacctagaagcttgtacactcgggcaatagtgaggcccacatccagtgggtcaagcccaaagcattcttgttccccggtatgatctcataagctgtacccacggctgaaagagtgattatcgttatcccactcagtacttcggagagcctagtacaccacttggaaatggaagtctgtgatccggggttgaccctgaaccccagaaactcatgatgaggctaaccttcccgaacacggcgacgtgtggttagcctgcgctggcatgaggcctctttgtaggcagactgaaatggaagggtgacgaagagccgactgagctactgttttattcctccggccccctgaatgcggctaatcctaactcctggtccagtacttgtaacccaacaggtggctggtcgtaatgcgtaagccgggagcggaaccgactactttggggcgtccgtgtttctcaatattattcatttctagcttatggtgacaatttatgattgcagagattgtgctgtatttgtgtctgagagaagaagtaacaat

SEQ ID NO: 681 - DNA, 619 bp, Unknown
  Description of Unknown Bat picornavirus isolate BtRs-PicoV sequence
tttcaaaaggccctgggcatacggcgttattcgtaacgtcgtatgtccagggcggtagcatcaggccaaggcctgatgctaccacgtgtggactaaaccacacactcttcttgtgacacgttgtgtcacctatccctttcttggtaacttagaagcttgtacacttacgcacgtaggtgccccacatccagtggggtttgtgcaaagcaatcttgttccccggtaaaccctgataggctgtaaccacggccgaaacaaggtttgtcgttacccgactcactactacacaaagcctagtaaagttcaatgaaagtgcgcagcgtgatccggtcaaaacccccttgaccagaaacacatgatgagggtcaccaacccccactggcgacagtgtggtgtccctgcgttggcatgtggcctcgtagaggcgttgcaatctggatttgctccgaagagccccgtgtgctagtgtttatacctccggccccttgaatgcggctaatcctaacccccgagcatgtacacacaagccagtgtgtagcatgtcgtaatgagcaatttggggatggaaccgactactttagggtgtccgtgtttctcattattctttgtttgatgtttt

SEQ ID NO: 682 - DNA, 593 bp, Unknown
  Description of Unknown Rhinolophus picornavirus strain Guizhou-Rr100 sequence
ttttttttctcaggcggtagcatccagccaaggcctgatgctaccaacgtgtgactaaaccacactctctttttgtgatacattgtgtcacctatccctttcttggtaacttagaagcttgtacacccacgcacgtaggtaccccacatccagtggggtttgtgcaaagcattcttgttccccggtaaaccctgataggctgtaaccacggctgaaacaaggtttgtcgttacccgactcactactacgcaaagcctagtaaagttcaatgaaagtgcgcagcgtgatccggtcaaaacccccttgaccagaaacacatgatgagggtcaccaacccccactggcgacagtgtggtgtccctgcgttggcatgtggcctcatagaggcgttgcaatctggatttgctccgaagagccccgtgtgctagtgtttatacctccggccccttgaatgcggctaatcctaacccccgagcatgtacacacacgccagtgtgcagcatgtcgtaatgagcaatttggggatggaaccgactactttagggtgtccgtgtttctcattattctttgtttgatgttttatggtgacaaca

SEQ ID NO: 683 - DNA, 597 bp, Unknown
  Description of Unknown Rhinolophus picornavirus strain Henan-Rf265 sequence
cggaacgttgtatgctcagggcgtaggcaccacccacgggtggtgcctacacgtgtggactaaaccacacactcttttcagcacttagtgctgctatctctttttgtaacttagaagtttgtacacaatgcgttagggccacacatccagtgtggtatcgcaaagcacttctgtttccccggtgctagtaggagggtggctgctccacggccacttgccgaacccatcgttacccgactcattacttcgcaaagcctagtaacccagttgaagcaagcccggcgtgttccggtcaggaaaaaccccccctggccagaaacatgtgatgagggtgggctatccccactggtgacagtgagccctccctgcgttggcacatggcccgatctgggcgtggttcttgtggatgctgccgaagagccccgtgagctagtgtttataccgccggcctcgtgaatgcggctaaccctaaccccggagcagaggctactgaagccacagtagtcgctgtcgtaacgagtaattctgggatgggaccgactactttcgagtgtccgtgtttcctttattcttttattgttgtttatggtgacaaac

SEQ ID NO: 684 - DNA, 636 bp, Enterovirus sp.
cccctaggatccactggatgtcagtacactggtatcgtggtacctttgtacgcctgttttatacccccttccccgcaactttagaagcatcaaaagcaccgctcaatagtcaccacacccccagtgtggtttcgagcaagcacttctgttttcccggttgcgtcccatatgctgtgcaaacggcaaaaagggacaatatcgttacccgcttgtatactacgggaaacctagtaccaccattgattgtgttgagagttgcgctcatcacctttccccggtgtagctcaggccgatgaggctcagaatcccccacaggtgactgtgtctgagcctgcgttggcggcctgccctcgccttatggcgtgggacgcttgatacatgacatggtgcgaagagtctactgtgctatgcaagagtcctccggcccctgaatgtggctaatcctaaccactgatcccacgcacgcaaaccagtgtgtagtgggtcgtaacgcgcaagtcggtggcggaaccaactactttgggtgaccgtgtttcctttattacttattgaatgtttatggtgacaattgtttgattcagttgttgccattctctacattcatttacccagcatcaaaccaattgaactgttaca

SEQ ID NO: 685 - DNA, 421 bp, Unknown
  Description of Unknown Human poliovirus 1 strain NIE1116623 sequence
agtctggacatccctcaccggcgacggtggtccaggctgcgctggcggcctacctgtggtccaaagccacgggacgctacatgtgaacaaggtgtgaagagcctattgagctacaaaagagtcctccggcccctgaatgcggctaatcccaaccacggatcaagggtgcacaaaccagtgtacaccttgtcgtaacgcgcaagtctgtggcggaaccgactactttgggtgtccgtgtttcctttttaattttgatggctgcttatggtgacaatcatagattgttatcataaagctaattggattggccatccggtgagagtgaaatatattgtttacctccctgttgggtttactctaactaacttctccatttataaacttgtcatcacagttttaataattagaagtgcagtttaca

SEQ ID NO: 686 - DNA, 663 bp, Enterovirus sp.
tttaaaacagcctgggggttgttcccacccccagggcccactgggcgttagtactctggtatcgcggtaccttagtatgcctgttttatgtctcctttcccccgcaactttagaagtaatcaagttatggctcaacagtcgccacacccccagtgtggttccgagcaagcacttctgttccccggttgcgtcttatatgctgtgtgaacggcagaaagggacaatatcgttatccgctcaactactacgggaagcctagtaccaccatggattgacctgaaagttgcgttcagcgcacccccagcgcagctcaggccgatgaggctccgaataccccacgggcgaccgtgtcggagcctgcgttggcggcctgcccacgttgcaaaacgtgggacgctcatttcatgacatggtgcgaagagcctactgtgctagttgagagtcctccggcccctgaatgtggataatcctaaccactgaacctacgggcgcaaaccagcgtctggtaggccgtaacgcgcaagtcggtggcggaaccaactactttgggtgtccgtgtttccttttatctttttgaatgtttatggtgacaattgttgtgtacagttgttaccatagtttgcattcagaaataaacctaacactttccaattatttgttaca

SEQ ID NO: 687 - DNA, 675 bp, Unknown
  Description of Unknown Human poliovirus 2 strain NIE0811460 sequence
ttgtgcgcctgttttatattccccccccgcaacttagaagcacgaaaccaagttcaatagaagggggtacaaaccagtaccaccacgaacaagcacttctgtttccccggtgacattgcatagactgctcacgcggttgaaagtgatcgatccgttacccgcttgtgtacttcgaaaagcctagtattgccttggaatcttcgacgcgttgcgctcagcacccgaccccggggtgtagcttaggctgatgagtctggacattcctcaccggtgacggtggtccaggctgcgttggcggcctacctatggctaatgccataggacgctagatgtgaacaaggtgtgaagagcctattgagctacataagagtcctccggcccctgaatgcggctaatcctaaccacggagcaggcggtcgcaaaccagtgactagcttgtcgtaacgcgcaagtctgtggcggaaccgactactttgggtgtccgtgtttcctgttatttttattatggctgcttatggtgacaatcagagattgttatcataaagcgaattggattggccatccggtgagtgttgtgtcaggtatacaactgtttgttggaaccactgtgttagtttaacctctctttcaaccaattagtcaaaaacaatacgaagatagaacaacaatactaca

SEQ ID NO: 688 - DNA, 512 bp, Unknown; identical to SEQ ID NO: 491
  Description of Unknown Bovine picornavirus sequence

SEQ ID NO: 689 - DNA, 675 bp, Unknown
  Description of Unknown Human poliovirus 1 strain EQG1419328 sequence
acccttgtacgcctgttttatactcccctccccgtaacttagaagaaacaaaataagttcaataggagggggtacaaaccagtaccaccacgaacaagcacttctgtctccccggtgacattgcatagactgtccccacggttgaaagcaattgatccgttacccgctcttgtacttcgagaagcctagtaccatcttggaatcatcgatgcgttgcgctccacactcagtcccagagtgtagcttaggctgatgagtctggacattcctcaccggcgacggtggtccaggctgcgttggcggcctacctgtggcccaaagccacaggacgctagatgtgaacaaggtgtgaagagcctattgagctataagagagtcctccggcccctgaatgcggctaatcccaaccacggatcaagggtgcacgaaccagtgtataccttgtcgtaacgcgcaagtccgtggcggaaccgactactttgggtgaccgtgtttccttttattatttcaatggctgcttatggtgacaatcattgattgttatcataaagcgaattggactggccatccggtgaaagtgaaacatattgtttgcctcctcgttgggtctacttcaaccaatctttacttacaatcttaccactacagttttgctggttagaagtgtgtttcacg

SEQ ID NO: 690 - DNA, 675 bp, Unknown
  Description of Unknown Human poliovirus 2 isolate IS_061 sequence
ttgtgcgcctgttttatactcccctcccgcaacttagaagcacgaaaccaagttcaatagaagggggtacaaaccagtaccactacgaacaagcacttctgtttccccggtgacattgcatagactgctcacgcggttgaaagtgatcgatccgttacccgcttgtgtacttcgaaaagcctagtatcgccttggaatcttcgacgcgttgcgctcagcacccgaccccggggtgtagcttaggccgatgagtctggacattcctcaccggtgacggtggtccaggctgcgttggcggcctacctatggctaacgccataggacgttagatgtgaacaaggtgtgaagagcctattgagctacataagagtcctccggcccctgaatgcggctaatcctaaccacggagcaggcggtcgcgaaccagtgactggcttgtcgtaacgcgcaagtctgtggcggaaccgactactttgggtgtccgtgtttcctgttatttttatcatggctgcttatggtgacaatcagagattgttatcataaagcgaattggattggccatccggtgagtgttgtgtcaggtatacaactgtttgttggaaccactgtgttagctttgcttctcatttaaccaattaatcaaaaacaatacgaggataaaacaacaatactaca

SEQ ID NO: 691 - DNA, 675 bp, Unknown
  Description of Unknown Coxsackievirus B5 sequence
cctttgtgcgcctgttttatgcccccttcccccaattgaaacttagaagttacacacaccgatcaacagcgggcgtggcataccagccgcgtcttgatcaagcactcctgtttccccggaccgagtatcaatagactgctcacgcggttgaaggagaaaacgttcgttacccggctaactacttcgagaaacctagtagcatcatgaaagttgcgaagcgtttcgctcagcacatccccagtgtagatcaggtcgatgagtcaccgcattccccacgggcgaccgtggcggtggctgcgttggcggcctgcctacggggcaacccgtaggacgcttcaatacagacatggtgcgaagagtcgattgagctagttagtagtcctccggcccctgaatccggctaatcctaactgcggagcacataccctcaacccagggggcattgtgtcgtaacgggtaactctgcagcggaaccgactactttgggtgtccgtgtttccttttattcttataatggctgcttatggtgacaattgaaagattgttaccatatagctattggattggccatccggtgtctaacagagctattatatacctctttgttggatttgtaccacttgatctaaaggaagtcaagacactacaattcatcatacaattgaacacagcaaa

SEQ ID NO: 692 - DNA, 675 bp, Unknown
  Description of Unknown Coxsackievirus A10 sequence
tttgtgcgcctgttttacaacccttccccaacttgtaacgtagaagtaatacacactactgatcaatagcaggcatggcgcgccagtcatgtctcgatcaagcacttctgttcccccggactgagtatcaatagactgctcacgcggttgaaggagaaaacgttcgttacccggctaactacttcgagaaacctagtagcaccatagaagctgcagagtgtttcgctcagcacttcccccgtgtagatcaggctgatgagtcactgcaatccccacgggtgaccgtggcagtggctgcgttggcggcctgcctatggggcaacccataggacgctctaatgtggacatggtgcgaagagtctattgagctagttagtagtcctccggcccctgaatgcggctaatcctaactgcggagcacatgccttcaacccagaaggtagtgtgtcgtaacgggcaactctgcagcggaaccgactactttgggtgtccgtgtttctttttattcctatattggctgcttatggtgacaatcacggaattgttgccatatagctattggattggccatccggtgtctaatagagctattgtgtacctatttgttggatttactccgctatcacataaatctctgaacactttgtgctttatattgaacttaaacacccgaaa

SEQ ID NO: 693 - protein, 18 aa, Unknown
  Description of Unknown Liver targeting moiety sequence
CKNEKKNIERNNKLKQPP

SEQ ID NO: 694 - DNA, 30 bp, Artificial Sequence
  Description of Artificial Sequence Synthetic oligonucleotide
tataattctaccctattgaggcattgacta

SEQ ID NO: 695 - protein, 493 aa, Artificial Sequence
  Description of Artificial Sequence Synthetic polypeptide
MALPVTALLLPLALLLHAARPDIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 696 - protein, 207 aa, Artificial Sequence
  Description of Artificial Sequence Synthetic polypeptide
KNQVEQSPQSLIILEGKNCTLQCNYTVSPFSNLRWYKQDTGRGPVSLTIMTFSENTKSNGRYTATLDADTKQSSLHITASQLSDSASYICVVNHSGGSYIPTFGRGTSLIVHPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 697 - protein, 246 aa, Artificial Sequence
  Description of Artificial Sequence Synthetic polypeptide
DVKVTQSSRYLVKRTGEKVFLECVQDMDHENMFWYRQDPGLGLRLIYFSYDVKMKEKGDIPEGYSVSREKKERFSLILESASTNQTSMYLCASSFLMTSGDPYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 698 - protein, 115 aa, Artificial Sequence
  Description of Artificial Sequence Synthetic polypeptide
MQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPY

SEQ ID NO: 699 - protein, 112 aa, Artificial Sequence
  Description of Artificial Sequence Synthetic polypeptide
MGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVL

SEQ ID NO: 700 - protein, 166 aa, Artificial Sequence
  Description of Artificial Sequence Synthetic polypeptide
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 701 - protein, 452 aa, Artificial Sequence
  Description of Artificial Sequence Synthetic polypeptide
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 702 - protein, 1462 aa, Artificial Sequence
  Description of Artificial Sequence Synthetic polypeptide
LSVKAQTAHIVLEDGTKMKGYSFGHPSSVAGEVVFNTGLGGYPEAITDPAYKGQILTMANPIIGNGGAPDTTALDELGLSKYLESNGIKVSGLLVLDYSKDYNHWLATKSLGQWLQEEKVPAIYGVDTRMLTKIIRDKGTMLGKIEFEGQPVDFVDPNKQNLIAEVSTKDVKVYGKGNPTKVVAVDCGIKNNVIRLLVKRGAEVHLVPWNHDFTKMEYDGILIAGGPGNPALAEPLIQNVRKILESDRKEPLFGISTGNLITGLAAGAKTYKMSMANRGQNQPVLNITNKQAFITAQNHGYALDNTLPAGWKPLFVNVNDQTNEGIMHESKPFFAVQFHPEVTPGPIDTEYLFDSFFSLIKKGKATTITSVLPKPALVASRVEVSKVLILGSGGLSIGQAGEFDYSGSQAVKAMKEENVKTVLMNPNIASVQTNEVGLKQADTVYFLPITPQFVTEVIKAEQPDGLILGMGGQTALNCGVELFKRGVLKEYGVKVLGTSVESIMATEDRQLFSDKLNEINEKIAPSFAVESIEDALKAADTIGYPVMIRSAYALGGLGSGICPNRETLMDLSTKAFAMTNQILVEKSVTGWKEIEYEVVRDADDNCVTVCNMENVDAMGVHTGDSVVVAPAQTLSNAEFQMLRRTSINVVRHLGIVGECNIQFALHPTSMEYCIIEVNARLSRSSALASKATGYPLAFIAAKIALGIPLPEIKNVVSGKTSACFEPSLDYMVTKIPRWDLDRFHGTSSRIGSSMKSVGEVMAIGRTFEESFQKALRMCHPSIEGFTPRLPMNKEWPSNLDLRKELSEPSSTRIYAIAKAIDDNMSLDEIEKLTYIDKWFLYKMRDILNMEKTLKGLNSESMTEETLKRAKEIGFSDKQISKCLGLTEAQTRELRLKKNIHPWVKQIDTLAAEYPSVTNYLYVTYNGQEHDVNFDDHGMMVLGCGPYHIGSSVEFDWCAVSSIRTLRQLGKKTVVVNCNPETVSTDFDECDKLYFEELSLERILDIYHQEACGGCIISVGGQIPNNLAVPLYKNGVKIMGTSPLQIDRAEDRSIFSAVLDELKVAQAPWKAVNTLNEALEFAKSVDYPCLLRPSYVLSGSAMNVVFSEDEMKKFLEEATRVSQEHPVVLTKFVEGAREVEMDAVGKDGRVISHAISEHVEDAGVHSGDATLMLPTQTISQGAIEKVKDATRKIAKAFAISGPFNVQFLVKGNDVLVIECNLRASRSFPFVSKTLGVDFIDVATKVMIGENVDEKHLPTLDHPIIPADYVAIKAPMFSWPRLRDADPILRCEMASTGEVACFGEGIHTAFLKAMLSTGFKIPQKGILIGIQQSFRPRFLGVAEQLHNEGFKLFATEATSDWLNANNVPATPVAWPSQEGQNPSLSSIRKLIRDGSIDLVINLPNNNTKFVHDNYVIRRTAVDSGIPLLTNFQVTKLFAEAVQKSRKVDSKSLFHYRQYSAGKAA

SEQ ID NO: 703 - protein, 1053 aa, Artificial Sequence
  Description of Artificial Sequence Synthetic polypeptide
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 704 - protein, 1353 aa, Artificial Sequence
  Description of Artificial Sequence Synthetic polypeptide
AAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 705 - protein, 431 aa, Artificial Sequence; identical to SEQ ID NO: 343
  Description of Artificial Sequence Synthetic polypeptide

SEQ ID NO: 706 - protein, 160 aa, Artificial Sequence; identical to SEQ ID NO: 337
  Description of Artificial Sequence Synthetic polypeptide

SEQ ID NO: 707 - protein, 133 aa, Artificial Sequence [sequence omitted]

SEQ ID NO: 708 - protein, 20 aa, Artificial Sequence
  Description of Artificial Sequence Synthetic peptide
  MISC_FEATURE (1)..(20): This sequence may encompass 1-10 “Gly Ser” repeating units
GSGSGSGSGSGSGSGSGSGS

SEQ ID NO: 709 - protein, 30 aa, Artificial Sequence
  Description of Artificial Sequence Synthetic polypeptide
  MISC_FEATURE (1)..(30): This sequence may encompass 1-10 “Gly Gly Ser” repeating units
GGSGGSGGSGGSGGSGGSGGSGGSGGSGGS

SEQ ID NO: 710 - protein, 40 aa, Artificial Sequence
  Description of Artificial Sequence Synthetic polypeptide
  MISC_FEATURE (1)..(40): This sequence may encompass 1-10 “Gly Gly Gly Ser” repeating units
GGGSGGGSGGGSGGGSGGGSGGGSGGGSGGGSGGGSGGGS

SEQ ID NO: 711 - protein, 40 aa, Artificial Sequence
  Description of Artificial Sequence Synthetic polypeptide
  MISC_FEATURE (1)..(40): This sequence may encompass 1-10 “Gly Gly Ser Gly” repeating units
GGSGGGSGGGSGGGSGGGSGGGSGGGSGGGSGGGSGGGSG

SEQ ID NO: 712 - protein, 50 aa, Artificial Sequence
  Description of Artificial Sequence Synthetic polypeptide
  MISC_FEATURE (1)..(50): This sequence may encompass 1-10 “Gly Gly Ser Gly Gly” repeating units
GGSGGGGSGGGGSGGGGSGGGGSGGGGSGGGGSGGGGSGGGGSGGGGSGG

SEQ ID NO: 713 - protein, 50 aa, Artificial Sequence
  Description of Artificial Sequence Synthetic polypeptide
  MISC_FEATURE (1)..(50): This sequence may encompass 1-10 “Gly Gly Gly Gly Ser” repeating units
GGGGSGGGGSGGGGSGGGGSGGGGSGGGGSGGGGSGGGGSGGGGSGGGGS

SEQ ID NO: 714 - protein, 50 aa, Artificial Sequence
  Description of Artificial Sequence Synthetic polypeptide
  MISC_FEATURE (1)..(50): This sequence may encompass 1-10 “Gly Gly Gly Gly Gly” repeating units
GGGGGGGGGGGGGGGGGGGGGGGGGGGGGGGGGGGGGGGGGGGGGGGGGG

SEQ ID NO: 715 - protein, 30 aa, Artificial Sequence
  Description of Artificial Sequence Synthetic polypeptide
  MISC_FEATURE (1)..(30): This sequence may encompass 1-10 “Gly Gly Gly” repeating units
GGGGGGGGGGGGGGGGGGGGGGGGGGGGGG

SEQ ID NO: 716 - protein, 20 aa, Artificial Sequence
  Description of Artificial Sequence Synthetic peptide
GGGGSGGGGSGGGGSGGGGS

SEQ ID NO: 717 - protein, 15 aa, Artificial Sequence
  Description of Artificial Sequence Synthetic peptide
GGGGSGGGGSGGGGS